(19) United States
(12) Patent Application Publication
(10) Pub. No.: US 2025/0282768 A1
(43) Pub. Date: Sep. 11, 2025

(54) PYRROLIDINONE DERIVATIVES AS INHIBITORS OF NF KAPPA B INDUCING KINASE

(71) Applicant: Janssen Pharmaceutica NV, Beerse (BE)

(72) Inventors: Alexander R. Rovira, San Diego, CA (US); Stephen K. Murphy, San Diego, CA (US); Alec D. Lebsack, Ladera Ranch, CA (US); Wenying Chai, San Diego, CA (US); Daniel Enny, San Diego, CA (US); Alexander Maertens, San Diego, CA (US); Michael Ameriks, San Diego, CA (US)

(73) Assignee: Janssen Pharmaceutica NV, Beerse (BE)

(21) Appl. No.: 18/863,761

(22) PCT Filed: May 10, 2023
(86) PCT No.: PCT/EP2023/062404

§ 371 Date: Nov. 7, 2024

Related U.S. Application Data
(60) Provisional application No. 63/364,549, filed on May 11, 2022.

(51) Int. Cl. C07D 413/14; A61K 31/497; A61K 31/506; A61K 31/519; A61K 31/53; A61K 31/5365; A61K 31/5377; A61K 31/5383; A61K 31/553; C07B 59/00; C07D 401/14; C07D 417/14; C07D 471/04; C07D 487/04; C07D 491/048; C07D 491/052; C07D 493/08; C07D 498/04

(52) U.S. Cl. CPC C07D 413/14; A61K 31/497; A61K 31/506; A61K 31/519; A61K 31/53; A61K 31/5365; A61K 31/5377; A61K 31/5383; A61K 31/553; C07B 59/002; C07D 401/14; C07D 417/14; C07D 471/04; C07D 487/04; C07D 491/048; C07D 491/052; C07D 493/08; C07D 498/04

(57) ABSTRACT

The present disclosure relates to compounds of Formula (I′) that inhibit NIK and pharmaceutical compositions comprising such compounds and methods of using the same. These compounds and pharmaceutical compositions are useful for preventing or treating diseases such as inflammatory disorders and autoimmune disorders.

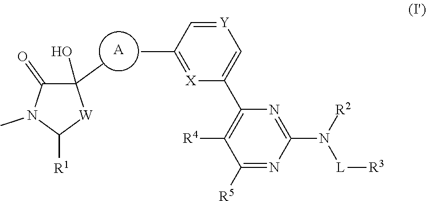

### CROSS-REFERENCE TO RELATED APPLICATIONS

This application claims the benefit of U.S. Provisional Application No. 63/364,549, filed on May 11, 2022, the entire disclosure of which is hereby incorporated herein by reference.

### FIELD

The present disclosure relates to the field of chemistry and medicine. More particularly, the present disclosure relates to NF-κB-inducing kinase inhibitors and their use in medical treatment.

### BACKGROUND

NF-κB inducing kinase (NIK) is a serine/threonine kinase transcription factor regulating the expression of various genes involved in immune response disorders. Because of this immune system regulatory role, inhibition of NIK blocks several downstream pathways that produce inflammatory molecules. Clinical validation with biologics has confirmed a key role for several NIK-dependent pathways in autoimmune diseases. See, e.g., S. V. Navarra, et al., The Lancet, 2011; 377(9767):721-31. One way to mitigate or eliminate the adverse effects associated with NIK activity is to increase NIK inhibition.

Thus, there is a need to develop effective NIK inhibitors that can be used to treat various diseases.

### SUMMARY

The present application discloses, inter alia, a compound of Formula I′:

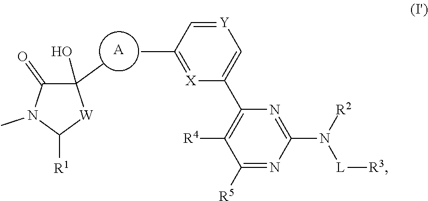

or a pharmaceutically acceptable salt thereof, wherein A, W, X, Y, R¹, R², L, R³, R⁴, and R⁵ are as defined herein.

The present application also discloses a compound of Formula I:

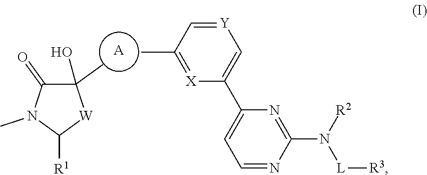

or a pharmaceutically acceptable salt thereof, wherein A, W, X, Y, R¹, R², L, and R³ are as defined herein.

The present application also discloses a pharmaceutical composition comprising a compound of Formula I′ or Formula I, or a pharmaceutically acceptable salt thereof, and a pharmaceutically acceptable carrier.

The present application also discloses a method for treating a disease, disorder, or medical condition mediated by NIK activity, comprising administering to a subject in need of such treatment an effective amount of (i) a compound of Formula I′ or Formula I, or a pharmaceutically acceptable carrier thereof, or (ii) a pharmaceutical composition comprising a compound of Formula I′ or Formula I, or a pharmaceutically acceptable salt thereof, and a pharmaceutically acceptable carrier. In some embodiments, the disease, disorder, or medical condition mediated by NIK activity is selected from the group consisting of inflammatory disorders, autoimmune disorders, cancers, metabolic disorders, and osteoporosis. In some embodiments, the disease, disorder, or medical condition mediated by NIK activity is selected from systemic lupus erythematosus (“SLE”), rheumatoid arthritis (“RA”), Sjogren's syndrome, lupus nephritis, inflammatory bowel disease (“IBD”), ANCA associated vasculitis, myositis, IgG4 associated diseases, bullous pemphigoid, neuromyelitis optica spectrum disorders (“NMOSD”), atopic dermatitis “AD”), hidradenitis supperativa (“HS”), steatosis, non-alcoholic steatohepatitis (“NASH”), primary biliary cirrhosis, leukemias, lymphomas, pancreatic cancer, breast cancer, melanoma, obesity, diabetes, acute kidney injury, IgAN, autosomal dominant polycystic kidney disease (“ADCKD”), membranous nephropathy, osteoporosis, bone resorption (periodontitis), multiple sclerosis (“MS”), immune thrombocytopenic purpura, transplantation, myasthenia gravis, scleroderma, myositis, IgG4 associated diseases, and bullous pemphigoid.

### DETAILED DESCRIPTION

Discussion of documents, acts, materials, devices, articles or the like which has been included in the present specification is for the purpose of providing context for the present disclosure. Such discussion is not an admission that any or all of these matters form part of the prior art with respect to any inventions disclosed or claimed.

NF-κB-inducing kinase (referred to as NIK, also known as MAP3K14) is a regulator and driver of the non-canonical NIK cascade, and thus represents an attractive target for therapeutic intervention. Embodiments described herein relate to compounds that inhibit NIK and pharmaceutical compositions comprising such compounds. Compounds described herein and pharmaceutical compositions thereof are useful for preventing or treating diseases such as inflammatory disorders and autoimmune disorders.

NIK-dependent transcriptional activation is a tightly controlled signaling pathway, through sequential events including phosphorylation and protein degradation. In a NIK activation pathway, known as a non-canonical pathway, activation is accomplished by phosphorylating the catalytic complex subunit IKKα, leading to the partial proteolysis of the gene product p100, liberating DNA-binding protein p52 which then heterodimerizes with another DNA-binding protein ReIB, translocates to the nucleus and mediates gene expression. The non-canonical pathway is activated by ligands such as CD40 ligands, B-cell activating factor (BAFF), lymphotoxin β receptor ligands, TNF-related weak inducer of apoptosis (TWEAK) cytokine, and receptor activator of nuclear factor kappa-B ligand (RANKL), also known as tumor necrosis factor ligand superfamily member 11 (TNFSF11). NIK has been shown to be required for activation of the pathway by these ligands (S.-C. Sun, Nat Rev Immunol. 2017, 17(9), 545-558). Because of its role, NIK expression is tightly regulated. Under normal non-stimulated conditions NIK protein levels are very low. This is due to its interaction with baculoviral-IAP-repeat-containing-3 (BIRC3, also known as CIAP2) and a range of TNF receptor associated factors (TRAF2 and TRAF3), which are ubiquitin ligases and result in degradation of NIK. It is believed that when the non-canonical pathway is stimulated by ligands under pathological/abnormal conditions, the activated receptors now compete for TRAFs, dissociating the TRAF-BIRC3-NIK complexes and thereby increasing the levels of NIK (For a more detailed analysis of this background, see e.g., S.-C. Sun (cited above) and Thu and Richmond, Cytokine Growth F. R. 2010, 21, 213-226). NIK plays a role propitiating immune response disorders, so a NIK level increase is undesirable, and one way to mitigate or eliminate the adverse effect associated with such increase is NIK inhibition.

BAFF/BAFF-R is a clinically validated therapeutic target whose inhibition is deemed beneficial for systemic lupus erythematosus (SLE) treatment. Belimumab (anti-BAFF antibody) has been approved to treat serum positive SLE patients (S. V. Navarra, et al., The Lancet, 2011; 377(9767):721-31). The CD40L/CD40 pathway plays a key role in T-dependent B cell activation, dendritic cell maturation and tissue inflammation/immunity (R. Elgueta, et al., Immunol. Rev. 2009; 229(1):152-72). An anti-CD40L antibody has demonstrated promising efficacy in phase 2 clinical studies in SLE patients (P. I. Sidiropoulos and D. T. Boumpas, Lupus 2004 May; 13(5):391-7). Mice lacking NIK (R. Shinkura, et al., Nature Genetics 1999; 22(1):74-7; H. D. Brightbill, et al., J Immunol. 2015; 195(3):953-64) or conditional knockout of NIK (H. D. Brightbill, et al., J Immunol. 2015; 195(3):953-64) or human patients carrying NIK gene mutations (K. L. Willmann, et al., Nature Comm. 2014; 5:5360) showed deficiency in NIK non-canonical activation pathways such as BAFF and CD40L pathway, reduced B lymphocytes in peripheral blood, and lymphoid organs and lower T cell dependent antibody responses supporting NIK as a therapeutic target for SLE.

NIK has been characterized as being “important in the immune and bone-destructive components of inflammatory arthritis and represents a possible therapeutic target for these diseases.” K. Aya, et al. (J. Clin. Invest. 2005, 115, 1848-1854). Mice lacking functional NIK have no peripheral lymph nodes, defective B and T cells, and impaired receptor activator of NIK ligand-stimulated osteoclastogenesis. K. Aya, et al. (J. Clin. Invest. 2005, 115, 1848-1854) investigated the role of NIK in murine models of inflammatory arthritis using NIK−/− mice. The serum transfer arthritis model was initiated by preformed antibodies and required only intact neutrophil and complement systems in recipients. While NIK−/− mice had inflammation equivalent to that of NIK+/+ controls, Ada, et al., (cited above) showed significantly less periarticular osteoclastogenesis and less bone erosion. In contrast, NIK−/− mice were completely resistant to antigen-induced arthritis (AIA), which requires intact antigen presentation and lymphocyte function but not lymph nodes. Additionally, transfer of NIK+/+ splenocytes or T cells to Rag2−/− mice conferred susceptibility to AIA, while transfer of NIK−/− cells did not. NIK−/− mice were also resistant to a genetic, spontaneous form of arthritis, generated in mice expressing both the KRN T cell receptor and H-2g7. Transgenic mice were used with OC-lineage expression of NIK lacking its TRAF3 binding domain (NT3), to demonstrate that constitutive activation of NIK drives enhanced osteoclastogenesis and bone resorption, both in basal conditions and in response to inflammatory stimuli. See Aya, et al., cited above. Furthermore, constitutive activation of NIK drives enhanced osteoclastogenesis and bone resorption, both in basal conditions and in response to inflammatory stimuli. (C. Yang, et al., PLoS ONE 2010, 5(11): e15383, doi:10.1371/journal.pone.0015383).

NIK is also a therapeutic target for other BAFF, CD40L or lymphotoxin β receptor ligands driven autoimmune disorders such as Sjogren's syndrome (J. Groom, et al., J. Clin. Invest. 2002; 109(1):59-68) and proliferative lupus glomerulonephritis (D. T. Boumpas, et al., Arthritis & Rheumatism 2003; 48(3):719-27).

### Definitions

Unless defined otherwise, all technical and scientific terms used herein have the same meaning as commonly understood to one of ordinary skill in the art to which the present disclosure pertains. Otherwise, certain terms used herein have the meanings as set forth in the specification.

It must be noted that as used herein and in the appended claims, the singular forms “a,” “an,” and “the” include plural reference unless the context clearly dictates otherwise.

As used herein, the terms “including,” “containing,” and “comprising” are used in their open, non-limiting sense.

To provide a more concise description, some of the quantitative expressions given herein are not qualified with the term “about.” It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

The term “administering” with respect to the methods of the present disclosure, means a method for therapeutically or prophylactically preventing, treating or ameliorating a syndrome, disorder or disease as described herein by using a compound of the disclosure, or pharmaceutically acceptable salt thereof, composition thereof, or medicament thereof. Such methods include administering a therapeutically effective amount of a compound of the disclosure, or pharmaceutically acceptable salt thereof, composition thereof, or medicament thereof, at different times during the course of a therapy or concurrently or sequentially as a combination therapy.

The term “subject” refers to a patient, which may be an animal, preferably a mammal, most preferably a human, whom will be or has been treated by a method according to an embodiment of the application. Examples of mammals include, but are not limited to, cows, horses, sheep, pigs, cats, dogs, mice, rats, rabbits, guinea pigs, non-human primates (NHPs) such as monkeys or apes, humans, etc., more preferably a human.

The term “therapeutically effective amount” or “effective amount” means that amount of active compound or pharmaceutical agent that elicits the biological or medicinal response in a tissue system, animal or human, that is being sought by a researcher, veterinarian, medical doctor, or other clinician, which includes preventing, treating or ameliorating the symptoms of a syndrome, disorder or disease being treated.

As used herein, the term “treatment” or “treating,” is defined as the application or administration of a therapeutic agent, i.e., a compound of the present disclosure (alone or in combination with another pharmaceutical agent), to a patient, or application or administration of a therapeutic agent to an isolated tissue or cell line from a patient (e.g., for diagnosis or ex vivo applications), who has a disorder or disease as described herein, a symptom thereof; or the potential to develop such disorder or disease, where the purpose of the application or administration is to cure, heal, alleviate, relieve, alter, remedy, ameliorate, improve or affect the disorder or disease, its symptoms, or the potential to develop said disorder or disease. Such treatments may be specifically tailored or modified, based on knowledge obtained from the field of pharmacogenomics.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

The term “C_{(a-b)}” (where a and b are integers referring to a designated number of carbon atoms) refers, for example, to an alkyl, alkenyl, alkynyl, alkoxy or cycloalkyl radical or to the alkyl portion of a radical in which alkyl appears as the prefix root containing from a to b carbon atoms inclusive. For example, C₍₁₋₄₎ denotes a radical containing 1, 2, 3 or 4 carbon atoms.

The term “alkyl” is a straight or branched saturated hydrocarbon. For example, an alkyl group can have 1 to 12 carbon atoms (i.e., (C₁-C₁₂)alkyl), 1 to 6 carbon atoms (i.e., (C₁-C₆)alkyl), 1 to 4 carbon atoms (i.e., (C₁-C₄)alkyl), or 1 to 3 carbon atoms (i.e., (C₁-C₃)alkyl). Examples of alkyl groups include, but are not limited to, methyl (Me, —CH₃), ethyl (Et, —CH₂CH₃), 1-propyl (n-Pr, n-propyl, —CH₂CH₂CH₃), isopropyl (i-Pr, i-propyl, —CH(CH₃)₂), 1-butyl (n-bu, n-butyl, —CH₂CH₂CH₂CH₃), 2-butyl (s-bu, s-butyl, —CH(CH₃)CH₂CH₃), tert-butyl (t-bu, t-butyl, —CH(CH₃)₃), 1-pentyl (n-pentyl, —CH₂CH₂CH₂CH₂CH₃), 2-pentyl (—CH(CH₃) CH₂CH₂CH₃), neopentyl (—CH₂C(CH₃)₃), 1-hexyl (—CH₂CH₂CH₂CH₂CH₂CH₃), 2-hexyl (—CH(CH₃)CH₂CH₂CH₂CH₃), heptyl (—(CH₂)₆CH₃), octyl (—(CH₂)₇CH₃), 2,2,4-trimethylpentyl (—CH₂C(CH₃)₂CH₂CH(CH₃)₂), nonyl (—(CH₂)₈CH₃), decyl (—(CH₂)₉CH₃), undecyl (—(CH₂)₁₀CH₃), and dodecyl (—(CH₂)₁₁CH₃). In an embodiment, alkyl refers to C₍₁₋₆₎alkyl. In another embodiment, alkyl refers to C₍₁₋₄₎alkyl. In another embodiment, alkyl refers to C₍₁₋₃₎alkyl.

The term “alkylene” refers to a linear or branched saturated divalent hydrocarbon moiety derived from an alkane having 1 to 12 carbon atoms (i.e., (C₁-C₁₂)alkylene), 1 to 6 carbon atoms (i.e., (C₁-C₆)alkylene), 1 to 4 carbon atoms (i.e., (C₁-C₄)alkylene), or 1 to 3 carbon atoms (i.e., (C₁-C₃)alkylene). Examples of alkylene groups include, but are not limited to, methylene (—CH₂—), ethylene (—CH₂CH₂—), —C(CH₃)H—, propylene (—CH₂CH₂CH₂—), isopropylene (—CH(CH₃)CH₂—), and —CH₂CH(CH₃)—. In an embodiment, alkylene refers to C₍₁₋₄₎alkylene. In another embodiment, alkylene refers to C₍₁₋₂₎alkylene.

The term “halo” or “halogen” refers to bromo (—Br), chloro (—Cl), fluoro (—F), or iodo (—I). In an embodiment, halo refers to fluoro.

The term “haloalkyl” refers to a straight- or branched-chain alkyl group having from 1 to 12 carbon atoms, 1 to 6 carbon atoms, 1 to 4 carbon atoms, or 1 to 3 carbon atoms in the chain optionally substituting one or more H with halo. Examples of “haloalkyl” groups include trifluoromethyl (CF₃), difluoromethyl (CF₂H), monofluoromethyl (CH₂F), pentafluoroethyl (CF₂CF₃), tetrafluoroethyl (CHFCF₃), monofluoroethyl (CH₂CH₂F), trifluoroethyl (CH₂CF₃), tetrafluorotrifluoromethylethyl (CF(CF₃)₂), and groups that in light of the ordinary skill in the art and the teachings provided herein would be considered equivalent to any one of the foregoing examples. In an embodiment, haloalkyl refers to C₍₁₋₆₎haloalkyl. In another embodiment, haloalkyl refers to C₍₁₋₄₎haloalkyl. In another embodiment, alkyl refers to C₍₁₋₃₎haloalkyl.

The term “cycloalkyl” refers to a saturated or partially unsaturated all carbon ring system having, for example, 3 to 10 carbon atoms (i.e., C₍₃₋₁₀₎cycloalkyl), 3 to 8 carbon atoms (i.e., C₍₃₋₈₎cycloalkyl), or 3 to 6 carbon atoms (i.e., C₍₃₋₆₎cycloalkyl), wherein the cycloalkyl ring system has a single ring or multiple rings in a fused, spirocyclic, or bridged configuration. Exemplary cycloalkyls include, but are not limited to, cyclopropyl, cyclobutyl, cyclopentyl, cyclohexyl, cycloheptyl, and cyclooctyl. Some cycloalkyl groups may exist as spirocycloalkyls, wherein two cycloalkyl rings are fused through a single carbon atom; for example and without limitation, an example of a spiropentyl group is

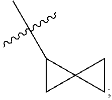 [SMILES: *C1CC12CC2]

for example and without limitation, examples of spirohexyl groups include

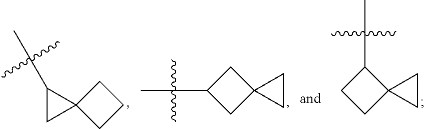 [SMILES: *C1CC12CCC2 ; *C1CC2(CC2)C1 ; *C1CCC12CC2]

and for example and without limitation examples of cycloheptyl groups include

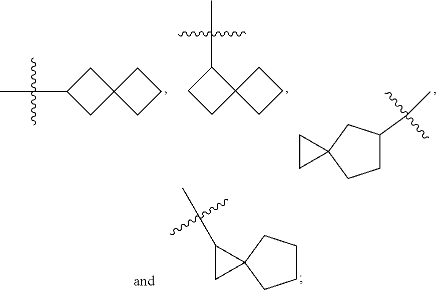

for example and without limitation examples of cyclooctyl groups include

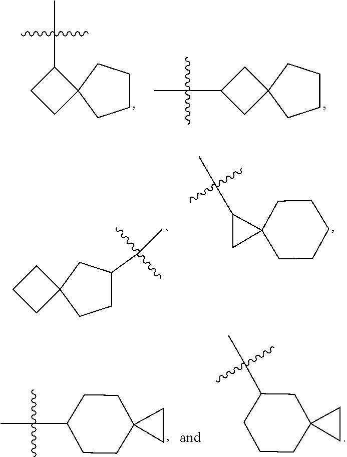 [SMILES: *C1CCC12CCCC2 ; *C1CC2(CCCC2)C1 ; *C1CCC2(CCC2)C1 ; *C1CC12CCCCC2 ; *C1CCC2(CC1)CC2 ; *C1CCCC2(CC2)C1]

In an embodiment, cycloalkyl refers to C₍₃₋₁₀₎cycloalkyl. In another embodiment, cycloalkyl refers to C₍₃₋₈₎cycloalkyl. In another embodiment, cycloalkyl refers to C₍₃₋₆₎cycloalkyl.

The term “cycloalkylene” refers to a divalent saturated or partially unsaturated all carbon ring system derived from a cycloalkane having, for example, 3 to 10 carbon atoms (i.e., C₍₃₋₁₀₎cycloalkylene), 3 to 8 carbon atoms (i.e., C₍₃₋₈₎cycloalkylene), or 3 to 6 carbon atoms (i.e., C₍₃₋₆₎cycloalkylene), wherein the cycloalkyl ring system has a single ring or multiple rings in a fused, spirocyclic, or bridged configuration. Examples of alkylene groups include, but are not limited to,

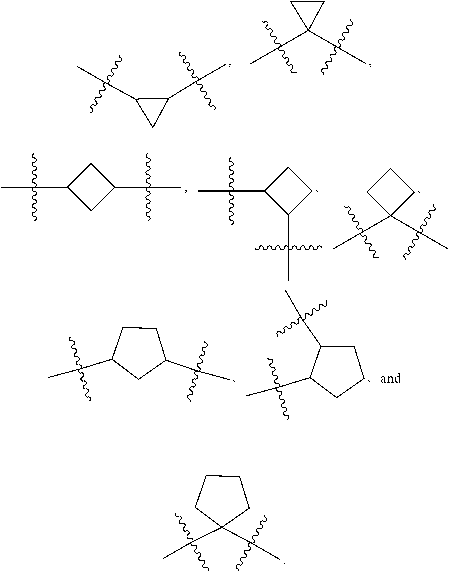

In an embodiment, cycloalkylene refers to C₍₃₋₁₀₎cycloalkylene. In another embodiment, cycloalkylene refers to C₍₃₋₈₎cycloalkylene. In another embodiment, cycloalkyl refers to C₍₃₋₆₎cycloalkylene.

The term “aryl,” unless otherwise stated, refers to a polyunsaturated, typically aromatic, hydrocarbon group which can be a single ring or multiple rings (up to three rings) which are fused together or linked covalently. The term aromatic is well known to a person skilled in the art and designates cyclically conjugated systems of 4n+2 electrons, that is with 6, 10, 14 etc. π-electrons (rule of Hückel). Examples of aryl groups include phenyl, naphthyl, anthracenyl. In an embodiment, aryl refers to C₍₆₋₁₀₎aryl. In another embodiment, aryl refers to phenyl.

The term “heterocyclyl” refers to a single saturated or partially unsaturated ring having 3 to 12 ring members, 3 to 10 ring members, 3 to 8 ring members, or 3 to 6 ring members and which contains carbon atoms and at least one atom other than carbon in the ring, wherein the atom is selected from the group consisting of N, O, and S. The terms “heterocyclyl” and “heterocycloalkyl” include cyclic esters (e.g., lactones) and cyclic amides (e.g., lactams). Exemplary heterocycles include, but are not limited to oxetanyl, aziridinyl, azetidinyl, pyrrolidinyl, pyrrolidonyl, piperidinyl, piperazinyl, morpholinyl, tetrahydropyranyl, tetrahydrofuranyl, and thiomorpholinyl. Unless otherwise noted, the heterocyclyl group is attached to its pendant group at any heteroatom or carbon atom that results in a stable structure. In an embodiment, heterocyclyl refers to 3- to 10-membered heterocyclyl. In another embodiment, heterocyclyl refers to 3- to 8-membered heterocyclyl. In another embodiment, heterocyclyl refers to 3- to 6-membered heterocyclyl.

As used herein, the term “5- to 12-membered bi- or tricyclic ring system containing one or more heteroatoms” refers to a saturated or partially saturated bridged polycyclic, fused polycyclic, or spiro polycyclic ring system having 5 to 12 ring members (or 7 to 12 ring members or 7 to 10 ring members) and which contains carbon atoms and from 1 to 7 heteroatoms, 1 to 5 heteroatoms, 1 to 4 heteroatoms, or 1 to 3 heteroatoms, wherein the heteroatoms are independently selected from the group consisting of N, O, and S. The ring system may include a fully aromatic ring; however, at least one other ring in the polycyclic ring system must be saturated or partially saturated. In some embodiments, the term refers to a fused bicyclic ring system. In some embodiments, the term refers to a fused bicyclic ring system wherein one of the rings is an aromatic ring. The term includes cyclic esters (e.g., lactones) and cyclic amides (e.g., lactams). Nonlimiting examples of 5- to 12-membered bi- or tricyclic ring system containing one or more heteroatoms include 3-oxabicyclo[3.1.0]hexyl, indolinyl, 6,7-dihydro-5H-cyclopenta[b]pyridinyl, 4,5,6,7-tetrahydro-1H-indazolyl, 6,7-dihydro-5H-cyclopenta[c]pyridazinyl, 4,5,6,7-tetrahydropyrazolo[1,5-a]pyridinyl, 4,5,6,7-tetrahydropyrazolo[1,5-a]pyrimidinyl, 3,4-dihydro-2H-pyrano[3,2-b]pyridinyl, 3-methyl-6,7-dihydro-5H-pyrazolo[5,1-b][1,3]oxazinyl, and 6,7-dihydropyrazolo[1,5-a]pyrazin-4(5H)-onyl. Unless otherwise noted, the bi- or tricyclic ring system is attached to its pendant group at any heteroatom or carbon atom that results in a stable structure. In an embodiment, a 5- to 12-membered bi- or tricyclic ring system containing one or more heteroatoms refers to a 5- to 12-membered bicyclic ring system containing one or more heteroatoms. In another embodiment, a 5- to 12-membered bi- or tricyclic ring system containing one or more heteroatoms refers to a 7- to 12-membered bicyclic ring system containing one or more heteroatoms. In another embodiment, a 5- to 12-membered bi- or tricyclic ring system containing one or more heteroatoms refers to a 7- to 10-membered bicyclic ring system containing one or more heteroatoms.

The term “heteroaryl” refers to a monocyclic or bicyclic aryl ring system having 5 to 12 ring members, 5 to 10 ring members, or 5 to 6 ring members, and which contains carbon atoms and from 1 to 5 heteroatoms, 1 to 3 heteroatoms, or 1 to 2 heteroatoms, wherein the heteroatoms are independently selected from the group consisting of N, O, and S. Included within the term heteroaryl are aromatic rings of 5 or 6 members wherein the ring consists of carbon atoms and has at least one heteroatom member. Suitable heteroatoms include nitrogen, oxygen, and sulfur. In the case of 5-membered rings, in some embodiments, the heteroaryl ring contains one member of nitrogen, oxygen or sulfur and, in addition, up to 3 additional nitrogens. In the case of 6-membered rings, in some embodiments, the heteroaryl ring contains from 1 to 3 nitrogen atoms. For the case wherein the 6-membered ring has 3 nitrogens, at most 2 nitrogen atoms are adjacent. Examples of heteroaryl groups include furyl, thienyl, pyrrolyl, oxazolyl, thiazolyl, imidazolyl, pyrazolyl, isoxazolyl, isothiazolyl, oxadiazolyl, triazolyl, thiadiazolyl, pyridinyl, pyridazinyl, pyrimidinyl, pyrazinyl, indolyl, isoindolyl, benzofuryl, benzothienyl, indazolyl, benzimidazolyl, benzothiazolyl, benzoxazolyl, benzisoxazolyl, benzothiadiazolyl, benzotriazolyl, quinolinyl, isoquinolinyl, quinazolinyl, pyrazolopyridinyl, and pyrazolopyrimidinyl. Those skilled in the art will recognize that the species of heteroaryl groups listed are not exhaustive, and that additional species within the scope of these defined terms may also be selected. Unless otherwise noted, the heteroaryl is attached to its pendant group at any heteroatom or carbon atom that results in a stable structure. In an embodiment, heteroaryl refers to 5- to 10-membered heteroaryl. In another embodiment, heteroaryl refers to 5- to 8-membered heteroaryl. In another embodiment, heteroaryl refers to 5- to 6-membered heteroaryl. In another embodiment, heteroaryl refers to 5-membered heteroaryl.

The term “substituted” means that the specified group or moiety bears one or more substituents. The term “unsubstituted” means that the specified group bears no substituents. The term “optionally substituted” means that the specified group is unsubstituted or substituted by one or more substituents. Where the term “substituted” is used to describe a structural system, the substitution is meant to occur at any valency-allowed position on the system.

Where the compounds disclosed herein have at least one stereocenter, they may accordingly exist as enantiomers or diastereomers. It is to be understood that all such isomers and mixtures thereof are encompassed within the scope of the present disclosure.

“Diastereoisomers” are stereoisomers that have at least two asymmetric atoms, but which are not mirror images of each other.

“Enantiomers” are a pair of stereoisomers that are non-superimposable mirror images of each other. A “racemic” mixture is a 1:1 mixture of a pair of enantiomers. A “scalemic” mixture of enantiomers is mixture of enantiomers at a ratio other than 1:1.

Where the processes for the preparation of the compounds according to the disclosure give rise to mixture of stereoisomers, these isomers may be separated by conventional techniques such as preparative chromatography. The compounds may be prepared in racemic form, a scalemic mixture, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-D-tartaric acid and/or (+)-di-p-toluoyl-L-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral column vial HPLC or SFC. In some instances rotamers of compounds may exist which are observable by ¹H NMR leading to complex multiplets and peak integration in the ¹H NMR spectrum.

The absolute stereochemistry is specified according to the Cahn-Ingold-Prelog R-S system. Chiral centers, of which the absolute configurations are known, are labelled by prefixes R and S, assigned by the standard sequence-rule procedure, and preceded when necessary by the appropriate locants (Pure & Appl. Chem. 45, 1976, 11-30). Certain pairs of enantiomers and diastereomers are presented together in the Examples. These enantiomers/diastereomers may be designated in the following synthetic method and characterized as enantiomer 1 or enantiomer 2 (or, alternately, diastereomer 1 or diastereomer 2). The presentation of stereoisomers in this manner conveys the separate preparation or isolation of the compounds as pure single enantiomers or diastereomers at the identified stereocenter(s). However, unless otherwise specified, when a pure single enantiomer (or diastereomer) is presented together with the corresponding pure single enantiomer (or diastereomer) in the Examples of the present disclosure, the order in which the chemical structures/IUPAC names are presented do not necessarily correspond to the order in which the Example numbers are listed. By way of example, where R and S enantiomers of a compound are presented side-by-side under the header “Example X and Example Y,” then Example X may be either the R enantiomer or the S enantiomer, and Example Y is the opposite enantiomer, regardless of the order in which the IUPAC names or chemical structures of the compounds are presented, unless otherwise specified in the method and characterization that follows.

Certain examples contain chemical structures that are depicted or labelled as an (*R) or (*S). When (*R*) or (*S) is used in the name of a compound or in the chemical representation of the compound, it is intended to convey that the compound is a pure single isomer at that stereocenter; however, absolute configuration of that stereocenter has not been established. Thus, a compound designated as (*R) refers to a compound that is a pure single isomer at that stereocenter with an absolute configuration of either (R) or (S), and a compound designated as (*S) refers to a compound that is a pure single isomer at that stereocenter with an absolute configuration of either (R) or (S). For example, (R)-3-Hydroxy-1-methyl-3-(3-(6-(2-(((*S)-6-methyl-6,7-dihydro-4H-pyrazolo[5,1-c][1,4]oxazin-2-yl)amino)pyrimidin-4-yl)pyridin-2-yl)isoxazol-5-yl)pyrrolidin-2-one:

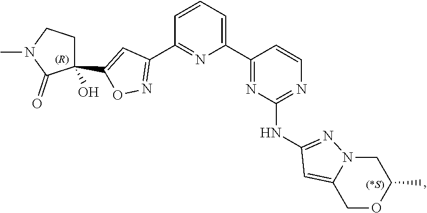

refers to a compound that is either:

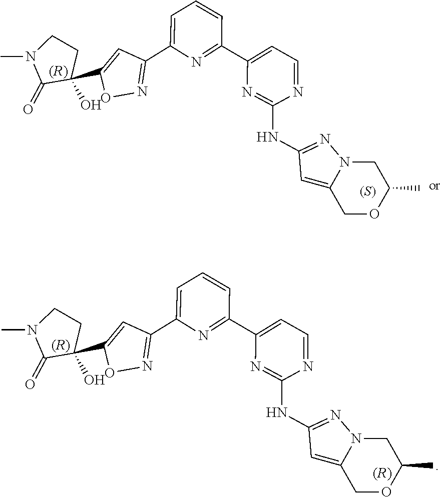

Pseudoasymmetric stereogenic centers are treated in the same way as chiral centers, but are given lower-case symbols, r or s (Angew. Chem. Int. Ed. Engl. 1982, 21, 567-583).

During any of the processes for preparation of the compounds disclosed herein, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known from the art.

Furthermore, it is intended that within the scope of the present disclosure, any element, in particular when mentioned in relation to a compound of the disclosure, or pharmaceutically acceptable salt thereof, shall comprise all isotopes and isotopic mixtures of said element, either naturally occurring or synthetically produced, either with natural abundance or in an isotopically enriched form. For example, a reference to hydrogen or “H” includes within its scope ¹H, ²H (i.e., deuterium or D), and ³H (i.e., tritium or T). In some embodiments, the compounds described herein include a ²H (i.e., deuterium) isotope. By way of example, the group denoted —C₍₁₋₆₎alkyl includes not only —C¹H³, but also C¹HD₂, C¹H₂D, CD₃ and other isotopic forms; not only C¹H₂C¹H₃, but also C¹HDC¹HD₂, C¹HDC¹H₂D, C¹H₂C¹H₃, CD₂CD₃, etc. Likewise, where nonexplicit hydrogen atoms are present in a chemical structure, those hydrogen atoms may be ¹H, ²H (i.e., deuterium or D), or ³H (i.e., tritium or T). By way of illustration, the group

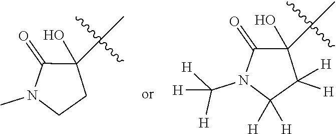

also encompasses

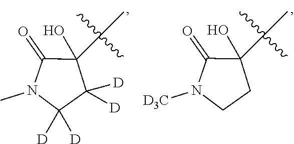 [SMILES: *C1(O)C(=O)N(C)C([2H])([2H])C1([2H])[2H] ; *C1(O)CCN(C([2H])([2H])[2H])C1=O]

etc. Similarly, references to carbon and oxygen include within their scope respectively ¹²C, ¹³C and ¹⁴C and ¹⁵O and ¹⁶O and ¹⁷O and ¹⁸O. The isotopes may be radioactive or non-radioactive. Radiolabelled compounds of the disclosure may include a radioactive isotope selected from the group comprising ³H, ¹¹C, ¹⁸F, ³⁵S, ¹²²I, ¹²³I, ¹²⁵I, ¹³¹I, ⁷⁵Br, ⁷⁶Br, ⁷⁷Br and ⁸²Br. In some embodiments, the radioactive isotope is selected from the group of ³H, ¹¹C and ¹⁸F.

Reference to a compound herein stands for a reference to any one of: (a) the actually recited form of such compound, and (b) any of the forms of such compound in the medium in which the compound is being considered when named. For example, reference herein to a compound such as R—COOH, encompasses reference to any one of, for example, R—COOH₍ₛ₎, R—COOH₍ₛₒₗ₎, and R—COO⁻₍ₛₒₗ₎. In this example, R—COOH₍ₛ₎ refers to the solid compound, as it could be for example in a tablet or some other solid pharmaceutical composition or preparation; R—COOH₍ₛₒₗ₎ refers to the undissociated form of the compound in a solvent; and R—COO⁻₍ₛₒₗ₎ refers to the dissociated form of the compound in a solvent, such as the dissociated form of the compound in an aqueous environment, whether such dissociated form derives from R—COOH, from a salt thereof, or from any other entity that yields R—COO⁻ upon dissociation in the medium being considered. In another example, an expression such as “exposing an entity to compound of formula R—COOH” refers to the exposure of such entity to the form, or forms, of the compound R—COOH that exists, or exist, in the medium in which such exposure takes place. In still another example, an expression such as “reacting an entity with a compound of formula R—COOH” refers to the reacting of (a) such entity in the chemically relevant form, or forms, of such entity that exists, or exist, in the medium in which such reacting takes place, with (b) the chemically relevant form, or forms, of the compound R—COOH that exists, or exist, in the medium in which such reacting takes place. In this regard, if such entity is for example in an aqueous environment, it is understood that the compound R—COOH is in such same medium, and therefore the entity is being exposed to species such as R—COOH_{aq)} and/or R—COO⁻_{(aq)}, where the subscript “(aq)” stands for “aqueous” according to its conventional meaning in chemistry and biochemistry. A carboxylic acid functional group has been chosen in these nomenclature examples; this choice is not intended, however, as a limitation but it is merely an illustration. It is understood that analogous examples can be provided in terms of other functional groups, including but not limited to hydroxyl, basic nitrogen members, such as those in amines, and any other group that interacts or transforms according to known manners in the medium that contains the compound. Such interactions and transformations include, but are not limited to, dissociation, association, tautomerism, solvolysis, including hydrolysis, solvation, including hydration, protonation, and deprotonation. No further examples in this regard are provided herein because these interactions and transformations in a given medium are known by any one of ordinary skill in the art.

The term “pharmaceutically acceptable” means approved or approvable by a regulatory agency of Federal or a state government or the corresponding agency in countries other than the United States, or that is listed in the U. S. Pharmcopoeia or other generally recognized pharmacopoeia for use in animals, and more particularly, in humans.

A “pharmaceutically acceptable salt” is intended to mean a salt of a free acid or base of a compound disclosed herein that is non-toxic, biologically tolerable, or otherwise biologically suitable for administration to the subject. It should possess the desired pharmacological activity of the parent compound. See, generally, G. S. Paulekuhn, et al., “Trends in Active Pharmaceutical Ingredient Salt Selection based on Analysis of the Orange Book Database”, J. Med. Chem., 2007, 50:6665-72, S. M. Berge, et al., “Pharmaceutical Salts”, J Pharm Sci., 1977, 66:1-19, and Handbook of Pharmaceutical Salts, Properties, Selection, and Use, Stahl and Wermuth, Eds., Wiley-VCH and VHCA, Zurich, 2002. Examples of pharmaceutically acceptable salts are those that are pharmacologically effective and suitable for contact with the tissues of patients without undue toxicity, irritation, or allergic response. A compound of the disclosure may possess a sufficiently acidic group, a sufficiently basic group, or both types of functional groups, and accordingly react with a number of inorganic or organic bases, and inorganic and organic acids, to form a pharmaceutically acceptable salt.

### Compounds of the Disclosure

The present application discloses a compound of Formula I′:

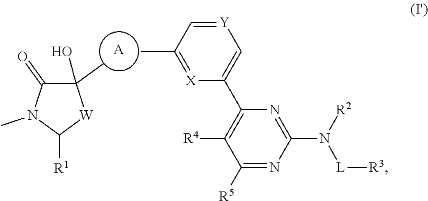

or a pharmaceutically acceptable salt thereof, wherein:
A is a 5-membered heteroaryl that is optionally substituted with one or more —C₍₁₋₄₎alkyl groups;
W is CH₂, CHF, CF₂, or CHR^{W};
X is N, C—H, or C—R^{X};
Y is N, C—H, or C—R^{Y};
R^{W} is —C₍₁₋₄₎alkyl or —C₍₁₋₄₎haloalkyl;
R^{X} is halo, —C₍₁₋₄₎alkyl, —C₍₁₋₄₎haloalkyl, —OC₍₁₋₄₎alkyl, —OC₍₁₋₄₎haloalkyl, or —C₍₁₋₄₎alkyl-O—C₍₁₋₄₎alkyl;
R^{Y} is halo, —C₍₁₋₄₎alkyl, —C₍₁₋₄₎haloalkyl, —OC₍₁₋₄₎alkyl, —OC₍₁₋₄₎haloalkyl, or —C₍₁₋₄₎alkyl-O—C₍₁₋₄₎alkyl;
R¹ is hydrogen, —C₍₁₋₄₎alkyl, or —C₍₁₋₄₎haloalkyl; or wherein R^{W} and R¹ are taken together with the carbon atoms to which they are attached to form a C₍₃₋₅₎cycloalkyl;
R² is hydrogen or —C₍₁₋₄₎alkyl;
L is absent, —C₍₁₋₄₎alkylene, or —C₍₃₋₆₎cycloalkylene, wherein the —C₍₁₋₄₎alkylene and —C₍₃₋₆₎cycloalkylene are optionally substituted with one to three groups selected from halo, —OH, —N(R^{N1})(R^{N2}), —C₍₁₋₃₎alkyl, —C₍₁₋₃₎haloalkyl, —C₍₃₋₅₎cycloalkyl, and —OC₍₁₋₃₎alkyl;
R³ is —C₍₁₋₁₀₎alkyl, —C₍₃₋₁₀₎cycloalkyl, 3- to 10-membered heterocyclyl, a 5- to 12-membered bi- or tricyclic ring system containing one or more heteroatoms, —C₍₆₋₁₀₎aryl, 5- to 10-membered heteroaryl, or —C(O)N(R^{N3})(R^{N4}), wherein the —C₍₁₋₁₀₎alkyl, —C₍₃₋₁₀₎cycloalkyl, 3- to 10-membered heterocyclyl, a 5- to 12-membered bi- or tricyclic ring system containing one or more heteroatoms, —C₍₆₋₁₀₎aryl, and 5- to 10-membered heteroaryl are each optionally substituted with one to five R^{3x} groups;
each R^{3x} independently for each occurrence is halo, —OH, —N(R^{N1})(R^{N2}), —CN, —C₍₁₋₈₎alkyl, —C₍₁₋₈₎haloalkyl, —C₍₃₋₈₎cycloalkyl, —OC₍₁₋₈₎alkyl, —OC₍₁₋₈₎haloalkyl, —OC₍₃₋₈₎cycloalkyl, —C₍₁₋₈₎alkylC₍₃₋₁₀₎cycloalkyl, —C₍₃₋₁₀₎cycloalkylC₍₁₋₈₎alkyl, —C(O)C₍₁₋₈₎alkyl, —S(O)₂C₍₁₋₈₎alkyl, —S(O)₂C₍₃₋₈₎cycloalkyl, —N(H)S(O)₂C₍₁₋₈₎alkyl, —C₍₀₋₈₎alkylC(O)N(R^{N1})(R^{N2}), 3- to 8-membered heterocyclyl, —C₍₁₋₈₎alkyl(3- to 8-membered heterocyclyl), —C₍₆₋₁₀₎aryl, or 5- to 10-membered heteroaryl, wherein the —C₍₁₋₈₎alkyl, —C₍₁₋₈₎ haloalkyl, —C₍₃₋₈₎cycloalkyl, —OC₍₁₋₈₎alkyl, —OC₍₁₋₈₎ haloalkyl, —OC₍₃₋₈₎cycloalkyl, —C₍₁₋₈₎alkylC₍₃₋₁₀₎cycloalkyl, —C₍₃₋₁₀₎cycloalkylC₍₁₋₈₎alkyl, —C(O)C₍₁₋₈₎alkyl, —S(O)₂C₍₁₋₈₎alkyl, —S(O)₂C₍₃₋₈₎cycloalkyl, —N(H)S(O)₂C₍₁₋₈₎alkyl, 3- to 8-membered heterocyclyl, —C₍₁₋₈₎alkyl(3- to 8-membered heterocyclyl), —C₍₆₋₁₀₎aryl, and 5- to 10-membered heteroaryl are optionally further substituted with one to five groups selected from halo, —OH, —N(R^{N1})(R^{N2}), —CN, —C₍₁₋₈₎alkyl, —C₍₁₋₈₎ haloalkyl, —OC₍₁₋₈₎alkyl, —OC₍₁₋₈₎ haloalkyl, and 3- to 5-membered heterocyclyl; or
R² and L-R³ are combined, along with the nitrogen atom to which they are attached, to form a 3- to 10-membered heterocyclyl, a 5- to 12-membered bicyclic ring system containing one or more heteroatoms, or a 5- to 10-membered heteroaryl, each of which is optionally further substituted with one to three groups selected from halo, —OH, —N(R_{N1})(R^{N2}), —CN, —C₍₁₋₈₎alkyl, and —OC₍₁₋₈₎alkyl wherein the —C₍₁₋₈₎alkyl and —OC₍₁₋₈₎alkyl are optionally further substituted with one to five halo groups;
R⁴ is hydrogen, halo, or —C₍₁₋₄₎alkyl;
R⁵ is hydrogen, halo, or —C₍₁₋₄₎alkyl;
R^{N1} and R^{N2} are each independently for each occurrence hydrogen, —C₍₁₋₈₎alkyl, or —C₍₁₋₈₎ haloalkyl;
R^{N3} is hydrogen or —C₍₁₋₈₎alkyl; and
R^{N4} is hydrogen —C₍₁₋₈₎alkyl, or phenyl; or R^{N3} and R^{N4} taken together with the nitrogen atom to which they are attached form a 3- to 10-membered heterocyclyl that is optionally substituted with one to five groups selected from halo, —OH, —N(R^{N1})(R^{N2}), —CN, —C₍₁₋₈₎alkyl, —C₍₁₋₈₎ haloalkyl, —OC₍₁₋₈₎alkyl, and —OC₍₁₋₈₎ haloalkyl.

In some embodiments, disclosed herein is the compound of Formula I′ as described herein above, wherein if R³ is —C(O)N(R^{N3})(R^{N4}), then L is not absent.

In some embodiments, disclosed herein is a compound of Formula I′, or a pharmaceutically acceptable salt thereof, wherein:
A is a 5-membered heteroaryl that is optionally substituted with one or more —C₍₁₋₄₎alkyl groups;
W is CH₂, CHF, CF₂, or CHR^{W};
X is N, C—H, or C—R^{X};
Y is N, C—H, or C—R^{Y};
R^{W} is —C₍₁₋₄₎alkyl or —C₍₁₋₄₎haloalkyl;
R^{X} is halo;
R^{Y} is halo, —C₍₁₋₄₎alkyl, —C₍₁₋₄₎haloalkyl, —OC₍₁₋₄₎alkyl, —OC₍₁₋₄₎haloalkyl, or —C₍₁₋₄₎alkyl-O—C₍₁₋₄₎alkyl;
R¹ is hydrogen, —C₍₁₋₄₎alkyl, or —C₍₁₋₄₎haloalkyl; or wherein R^{W} and R¹ are taken together with the carbon atoms to which they are attached to form a C₍₃₋₅₎cycloalkyl;
R² is hydrogen or —C₍₁₋₄₎alkyl;
L is absent, —C₍₁₋₄₎alkylene, or —C₍₃₋₆₎cycloalkylene, wherein the —C₍₁₋₄₎alkylene and —C₍₃₋₆₎cycloalkylene are optionally substituted with one to three groups selected from halo, —OH, —N(R^{N1})(R^{N2}), —C₍₁₋₃₎alkyl, —C₍₁₋₃₎haloalkyl, —C₍₃₋₅₎cycloalkyl, and —OC₍₁₋₃₎alkyl;
R³ is —C₍₁₋₆₎alkyl, —C₍₃₋₁₀₎cycloalkyl, 3- to 10-membered heterocyclyl, a 5- to 12-membered bi- or tricyclic ring system containing one or more heteroatoms, —C₍₆₋₁₀₎aryl, 5- to 10-membered heteroaryl, or —C(O)N(R^{N3})(R^{N4}), wherein the —C₍₁₋₆₎alkyl, —C₍₃₋₁₀₎cycloalkyl, 3- to 10-membered heterocyclyl, a 5- to 12-membered bi- or tricyclic ring system containing one or more heteroatoms, —C₍₆₋₁₀₎aryl, and 5- to 10-membered heteroaryl are each optionally substituted with one to five R^{3x} groups;
each R^{3x} independently for each occurrence is halo, —OH, —N(R^{N1})(R^{N2}), —CN, —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, —C₍₁₋₃₎alkylC₍₃₋₁₀₎cycloalkyl, —C₍₃₋₁₀₎cycloalkylC₍₁₋₃₎alkyl, —C(O)C₍₁₋₄₎alkyl, —S(O)₂C₍₁₋₄₎alkyl, —S(O)₂C₍₃₋₈₎cycloalkyl, —N(H)S(O)₂C₍₁₋₄₎alkyl, —C₍₀₋₃₎alkylC(O)N(R^{N1})(R^{N2}), 3- to 8-membered heterocyclyl, —C₍₁₋₃₎alkyl(3- to 8-membered heterocyclyl), —C₍₆₋₁₀₎aryl, or 5- to 10-membered heteroaryl, wherein the —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, —C₍₁₋₃₎alkylC₍₃₋₁₀₎cycloalkyl, —C₍₃₋₁₀₎cycloalkylC₍₁₋₃₎alkyl, —C(O)C₍₁₋₄₎alkyl, —S(O)₂C₍₁₋₄₎alkyl, —S(O)₂C₍₃₋₈₎cycloalkyl, —N(H)S(O)₂C₍₁₋₄₎alkyl, 3- to 8-membered heterocyclyl, —C₍₁₋₃₎alkyl(3- to 8-membered heterocyclyl), —C₍₆₋₁₀₎aryl, and 5- to 10-membered heteroaryl are optionally further substituted with one to five groups selected from halo, —OH, —N(R^{N1})(R^{N2}), —CN, —C₍₁₋₄₎alkyl, —C₍₁₋₄₎haloalkyl, —OC₍₁₋₄₎alkyl, —OC₍₁₋₄₎ haloalkyl, and 3- to 5-membered heterocyclyl; or
R² and L-R³ are combined, along with the nitrogen atom to which they are attached, to form a 3- to 10-membered heterocyclyl, a 5- to 12-membered bicyclic ring system containing one or more heteroatoms, or a 5- to 10-membered heteroaryl, each of which is optionally further substituted with one to three groups selected from halo, —OH, —N(R^{N1})(R^{N2}), —CN, —C₍₁₋₆₎alkyl, and —OC₍₁₋₆₎alkyl wherein the —C₍₁₋₆₎alkyl and —OC₍₁₋₆₎alkyl are optionally further substituted with one to five halo groups;
R⁴ is hydrogen, halo, or —C₍₁₋₄₎alkyl;
R⁵ is hydrogen, halo, or —C₍₁₋₄₎alkyl;
R^{N1} and R^{N2} are each independently for each occurrence hydrogen, —C₍₁₋₃₎alkyl, or —C₍₁₋₃₎haloalkyl;
R^{N3} is hydrogen or —C₍₁₋₄₎alkyl; and
R^{N4} is hydrogen —C₍₁₋₄₎alkyl or phenyl; or R^{N3} and R^{N4} taken together with the nitrogen atom to which they are attached form a 3- to 10-membered heterocyclyl that is optionally substituted with one to five groups selected from halo, —OH, —N(R^{N1})(R^{N2}), —CN, —C₍₁₋₄₎alkyl, —C₍₁₋₄₎haloalkyl, —OC₍₁₋₄₎alkyl, and —OC₍₁₋₄₎ haloalkyl;
wherein if R³ is —C(O)N(R^{N3})(R^{N4}), then L is not absent.

In some embodiments, disclosed herein is a compound of Formula I′, or a pharmaceutically acceptable salt thereof, wherein:
A is a 5-membered heteroaryl that is optionally substituted with one-C₍₁₋₄₎alkyl group;
W is CH₂, CF₂, or CHR^{W};
X is N, C—H, or C—R^{X};
Y is N, C—H, or C—R^{Y};
R^{W} is —C₍₁₋₄₎alkyl, —C₍₁₋₄₎haloalkyl, or R^{W} and R¹ are taken together with the carbon atoms to which they are attached to form a C₍₃₋₅₎cycloalkyl;
R^{X} is halo;
R^{Y} is halo, —C₍₁₋₄₎alkyl, —C₍₁₋₄₎haloalkyl, —OC₍₁₋₄₎alkyl, —OC₍₁₋₄₎ haloalkyl, or —C₍₁₋₄₎alkyl-O—C₍₁₋₄₎alkyl;
R¹ is hydrogen, —C₍₁₋₄₎alkyl, or —C₍₁₋₄₎haloalkyl;
R² is hydrogen;
L is absent, —C₍₁₋₄₎alkylene, or —C₍₃₋₆₎cycloalkylene, wherein the —C₍₁₋₄₎alkylene and —C₍₃₋₆₎cycloalkylene are optionally substituted with one to three groups selected from halo, —OH, —N(R^{N1})(R^{N2}), —C₍₁₋₃₎alkyl, —C₍₁₋₃₎haloalkyl, —C₍₃₋₅₎cycloalkyl, and —OC₍₁₋₃₎alkyl;
R³ is —C₍₁₋₆₎alkyl, —C₍₃₋₁₀₎cycloalkyl, 3- to 10-membered heterocyclyl, a 5- to 12-membered bi- or tricyclic ring system containing one or more heteroatoms, —C₍₆₋₁₀₎aryl, 5- to 10-membered heteroaryl, or —C(O)N(R^{N3})(R^{N4}), wherein: the —C₍₁₋₆₎alkyl is optionally substituted with one to five R^{3a} groups; the —C₍₃₋₁₀₎cycloalkyl is optionally substituted with one to five R^{3b} groups; the 3- to 10-membered heterocyclyl is optionally substituted with one to five R^{3c} groups; the 5- to 12-membered bi- or tricyclic ring system containing one or more heteroatoms is optionally substituted with one to five R^{3d}; the —C₍₆₋₁₀₎aryl is optionally substituted with one to five R^{3e} groups; and the 5- to 10-membered heteroaryl is optionally substituted with one to five R^{3f} groups;
each R^{3a} independently for each occurrence is halo, —OH, —N(R^{N1})(R^{N2}), —CN, —C₍₃₋₈₎cycloalkyl, —OC₍₁₋₆₎alkyl, —OC₍₁₋₆₎haloalkyl, —OC₍₃₋₈₎cycloalkyl, —S(O)₂C₍₁₋₄₎alkyl, —N(H)S(O)₂C₍₁₋₄₎alkyl, or 3- to 8-membered heterocyclyl, wherein the —C₍₃₋₈₎cycloalkyl, —OC₍₁₋₆₎alkyl, —OC₍₁₋₆₎haloalkyl, —OC₍₃₋₈₎cycloalkyl, and 3- to 8-membered heterocyclyl are optionally further substituted with one to five groups selected from halo, —OH, —N(R^{N1})(R^{N2}), —CN, —C₍₁₋₄₎alkyl, —C₍₁₋₄₎haloalkyl, —OC₍₁₋₄₎alkyl, and —OC₍₁₋₄₎ haloalkyl;
R^{3b}, R^{3c}, and R^{3d} are each independently for each occurrence halo, —OH, —N(R^{N1})(R^{N2}), —CN, —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —C₍₁₋₃₎alkylC₍₃₋₁₀₎cycloalkyl, —C(O)C₍₁₋₄₎alkyl, —S(O)₂C₍₁₋₄₎alkyl, —S(O)₂C₍₃₋₈₎cycloalkyl, —N(H)S(O)₂C₍₁₋₄₎alkyl, —C₍₀₋₃₎alkylC(O)N(R^{N1})(R^{N2}), 3- to 8-membered heterocyclyl, or 5- to 10-membered heteroaryl, wherein the —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, 3- to 8-membered heterocyclyl, and 5- to 10-membered heteroaryl are optionally further substituted with one to five groups selected from halo, —OH, —N(R^{N1})(R^{N2}), —CN, —C₍₁₋₄₎alkyl, —C₍₁₋₄₎haloalkyl, —OC₍₁₋₄₎alkyl, and —OC₍₁₋₄₎haloalkyl;
R^{3e} and R^{3f} are each independently for each occurrence halo, —OH, —N(R^{N1})(R^{N2}), —CN, —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₃₋₈₎cycloalkyl, —C₍₁₋₃₎alkylC₍₃₋₁₀₎cycloalkyl, —C(O)C₍₁₋₄₎alkyl, —S(O)₂C₍₁₋₄₎alkyl, —N(H)S(O)₂C₍₁₋₄₎alkyl, —C₍₀₋₃₎alkylC(O)N(R^{N1})(R^{N2}), 3- to 8-membered heterocyclyl, —C₍₁₋₃₎alkyl(3- to 8-membered heterocyclyl), —C₍₆₋₁₀₎aryl, or 5- to 10-membered heteroaryl, wherein the —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, —C₍₁₋₃₎alkylC₍₃₋₁₀₎cycloalkyl, 3- to 8-membered heterocyclyl, —C₍₁₋₃₎alkyl(3- to 8-membered heterocyclyl), —C₍₆₋₁₀₎aryl, and 5- to 10-membered heteroaryl are optionally further substituted with one to five groups selected from halo, —OH, —N(R^{N1})(R^{N2}), —CN, —C₍₁₋₄₎alkyl, —C₍₁₋₄₎haloalkyl, —OC₍₁₋₄₎alkyl, —OC₍₁₋₄₎ haloalkyl, and 3- to 5-membered heterocyclyl; or
R² and L-R³ are combined, along with the nitrogen atom to which they are attached, to form a 3- to 10-membered heterocyclyl, a 5- to 12-membered bicyclic ring system containing one or more heteroatoms, or a 5- to 10-membered heteroaryl, each of which that is optionally substituted with one to three groups selected from halo, —OH, —N(R^{N1})(R^{N2}), —CN, —C₍₁₋₆₎alkyl, —C₍₁₋₆₎haloalkyl, —OC₍₁₋₆₎alkyl, and —OC₍₁₋₆₎haloalkyl;
R⁴ is hydrogen, halo, or —C₍₁₋₄₎alkyl;
R⁵ is hydrogen, halo, or —C₍₁₋₄₎alkyl;
R^{N1} and R^{N2} are each independently for each occurrence hydrogen or —C₍₁₋₃₎alkyl;
R^{N3} is hydrogen or —C₍₁₋₄₎alkyl; and
R^{N4} is hydrogen —C₍₁₋₄₎alkyl or phenyl; or R^{N3} and R^{N4} taken together with the nitrogen atom to which they are attached form a 3- to 10-membered heterocyclyl that is optionally substituted with one to five groups selected from halo, —OH, —N(R^{N1})(R^{N2}), —CN, —C₍₁₋₄₎alkyl, —C₍₁₋₄₎haloalkyl, —OC₍₁₋₄₎alkyl, and —OC₍₁₋₄₎ haloalkyl;
wherein if R³ is —C(O)N(R^{N3})(R^{N4}), then L is not absent.

In some embodiments, disclosed herein is a compound of Formula I′, or a pharmaceutically acceptable salt thereof, wherein:
A is a 5-membered heteroaryl that is optionally substituted with one —C₍₁₋₄₎alkyl group;
W is CH₂, CF₂, or CHR^{W};
X is N, C—H, or C—R^{X};
Y is N, C—H, or C—R^{Y};
R^{W} is —C₍₁₋₄₎alkyl, —C₍₁₋₄₎haloalkyl, or R^{W} and R¹ are taken together with the carbon atoms to which they are attached to form a C₍₃₋₅₎cycloalkyl;
R^{X} is halo;
R^{Y} is —C₍₁₋₄₎alkyl, —C₍₁₋₄₎haloalkyl, —OC₍₁₋₄₎alkyl, or —C₍₁₋₄₎alkyl-O—C₍₁₋₄₎alkyl;
R¹ is hydrogen, —C₍₁₋₄₎alkyl, or —C₍₁₋₄₎haloalkyl;
R² is hydrogen;
L is absent, —C₍₁₋₄₎alkylene, or —C₍₃₋₆₎cycloalkylene, wherein the —C₍₁₋₄₎alkylene and —C₍₃₋₆₎cycloalkylene are optionally substituted with one to three groups selected from halo, —OH, —N(R^{N1})(R^{N2}), —C₍₁₋₃₎alkyl, —C₍₁₋₃₎haloalkyl, —C₍₃₋₅₎cycloalkyl, and —OC₍₁₋₃₎alkyl;
R³ is —C₍₁₋₆₎alkyl, —C₍₃₋₁₀₎cycloalkyl, 3- to 10-membered heterocyclyl, a 5- to 12-membered bi- or tricyclic ring system containing one or more heteroatoms, —C₍₆₋₁₀₎aryl, 5- to 10-membered heteroaryl, or —C(O)N(R^{N3})(R^{N4}), wherein: the —C₍₁₋₆₎alkyl is optionally substituted with one to five R^{3a} groups; the —C₍₃₋₁₀₎cycloalkyl is optionally substituted with one to five R^{3b} groups; the 3- to 10-membered heterocyclyl is optionally substituted with one to five R^{3c} groups; the 5- to 12-membered bi- or tricyclic ring system containing one or more heteroatoms is optionally substituted with one to five R^{3d}; and the 5- to 10-membered heteroaryl is optionally substituted with one to five R^{3f} groups;
each R^{3a} independently for each occurrence is halo, —OH, —N(R^{N1})(R^{N2}), —CN, —OC₁₋₆₎alkyl, or —OC₍₁₋₆₎haloalkyl;
R^{3b}, R^{3c}, and R^{3d} are each independently for each occurrence halo, —OH, —N(R^{N1})(R^{N2}), —CN, —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —C₍₁₋₃₎alkylC₍₃₋₁₀₎cycloalkyl, —S(O)₂C₍₁₋₄₎alkyl, or —S(O)₂C₍₃₋₈₎cycloalkyl wherein the —C₍₁₋₆₎alkyl, —C₍₁₋₆₎haloalkyl, —OC₍₁₋₆₎alkyl, and —OC₍₁₋₆₎haloalkyl are optionally further substituted with one to five groups selected from halo, —OH, —N(R^{N1})(R^{N2}), —CN, and —OC₍₁₋₄₎alkyl;
each R^{3f} is independently for each occurrence halo, —OH, —N(R^{N1})(R^{N2}), —CN, —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, —C₍₁₋₃₎alkylC₍₃₋₁₀₎cycloalkyl, —C₍₀₋₃₎alkylC(O)N(R^{N1})(R^{N2}), 3- to 8-membered heterocyclyl, —C₍₁₋₃₎alkyl(3- to 8-membered heterocyclyl), —C₍₆₋₁₀₎aryl, or 5- to 10-membered heteroaryl, wherein the —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, —C₍₁₋₃₎alkylC₍₃₋₁₀₎cycloalkyl, 3- to 8-membered heterocyclyl, —C₍₁₋₃₎alkyl(3- to 8-membered heterocyclyl), —C₍₆₋₁₀₎aryl, and 5- to 10-membered heteroaryl are optionally further substituted with one to five groups selected from halo, —OH, —N(R^{N1})(R^{N2}), —CN, —C₍₁₋₄₎alkyl, —C₍₁₋₄₎haloalkyl, —OC₍₁₋₄₎alkyl, —OC₍₁₋₄₎ haloalkyl, and 3- to 5-membered heterocyclyl; or
R² and L-R³ are combined, along with the nitrogen atom to which they are attached, to form a 5- to 12-membered bicyclic ring system containing one or more heteroatoms or a 5- to 10-membered heteroaryl, each of which that is optionally further substituted with one to three groups selected from halo, —OH, —N(R^{N1})(R^{N2}), —CN, —C₍₁₋₆₎alkyl, —C₍₁₋₆₎haloalkyl, —OC₍₁₋₆₎alkyl and —OC₍₁₋₆₎haloalkyl;
R⁴ is hydrogen, halo, or —C₍₁₋₄₎alkyl;
R⁵ is hydrogen, halo, or —C₍₁₋₄₎alkyl;
R^{N1} and R^{N2} are each independently for each occurrence hydrogen or —C₍₁₋₃₎alkyl;
R^{N3} is hydrogen or —C₍₁₋₄₎alkyl; and
R^{N4} is hydrogen —C₍₁₋₄₎alkyl or phenyl; or R^{N3} and R^{N4} taken together with the nitrogen atom to which they are attached form a 3- to 10-membered heterocyclyl that is optionally substituted with one to five groups selected from halo, —OH, —N(R^{N1})(R^{N2}), —CN, —C₍₁₋₄₎alkyl, —C₍₁₋₄₎haloalkyl, —OC₍₁₋₄₎alkyl, and —OC₍₁₋₄₎ haloalkyl;
wherein if R³ is —C(O)N(R^{N3})(R^{N4}), then L is not absent.

In some embodiments, disclosed herein is a compound of Formula I′, or a pharmaceutically acceptable salt thereof, wherein:
A is a 5-membered heteroaryl that is optionally substituted with one —CH₃ group;
W is CH₂, CF₂, or CHR^{W};
X is N, C—H, or C—R^{X};
Y is N, C—H, or C—R^{Y};
R^{W} is —C₍₁₋₄₎alkyl, —C₍₁₋₄₎haloalkyl, or R^{W} and R¹ are taken together with the carbon atoms to which they are attached to form a C₍₃₋₅₎cycloalkyl;
R^{X} is halo;
R^{Y} is —C₍₁₋₄₎alkyl, —C₍₁₋₄₎haloalkyl, —OC₍₁₋₄₎alkyl, or —C₍₁₋₄₎alkyl-O—C₍₁₋₄₎alkyl;
R¹ is hydrogen, —C₍₁₋₄₎alkyl, or —C₍₁₋₄₎haloalkyl;
R² is hydrogen;
L is absent, —C₍₁₋₄₎alkylene, or —C₍₃₋₆₎cycloalkylene, wherein the —C₍₁₋₄₎alkylene is optionally substituted with one to three groups selected from halo, —OH, —N(R^{N1})(R^{N2}), and cyclopropyl;
R³ is —C₍₁₋₆₎alkyl, —C₍₃₋₁₀₎cycloalkyl, 3- to 10-membered heterocyclyl, a 5- to 12-membered bi- or tricyclic ring system containing one or more heteroatoms, phenyl, 5- to 10-membered heteroaryl, or —C(O)N(R^{N3})(R^{N4}); wherein: the —C₍₁₋₆₎alkyl is optionally substituted with one to five R^{3a} groups; the —C₍₃₋₁₀₎cycloalkyl is optionally substituted with one to five R^{3b} groups; the 3- to 10-membered heterocyclyl is optionally substituted with one to five R^{3c} groups; the 5- to 12-membered bi- or tricyclic ring system containing one or more heteroatoms is optionally substituted with one to five R^{3d}; and the 5- to 10-membered heteroaryl is optionally substituted with one to five R^{3f} groups;
each R^{3a} independently for each occurrence is halo, —OH, —OC₍₁₋₆₎alkyl, or —OC₍₁₋₆₎haloalkyl;
each R^{3b} independently for each occurrence is halo, —OH, —C₍₁₋₆₎haloalkyl, or —OC₍₁₋₆₎alkyl;
each R^{3c} independently for each occurrence is halo, —OH, —N(R^{N1})(R^{N2}), —CN, —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —C₍₁₋₃₎alkylC₍₃₋₁₀₎cycloalkyl, —S(O)₂C₍₁₋₄₎alkyl, or —S(O)₂C₍₃₋₈₎cycloalkyl wherein the —C₍₁₋₆₎alkyl and —OC₍₁₋₆₎alkyl are optionally further substituted with one to five groups selected from halo and —OC₍₁₋₄₎alkyl;
each R^{3d} is independently for each occurrence halo, —OH, —N(R^{N1})(R^{N2}), —CN, —C₍₁₋₆₎alkyl, —C₍₁₋₆₎haloalkyl, —OC₍₁₋₆₎alkyl, —OC₍₁₋₆₎haloalkyl, or —C₍₁₋₃₎alkylC₍₃₋₁₀₎cycloalkyl;
each R^{3f} is independently for each occurrence halo, —OH, —N(R^{N1})(R^{N2}), —CN, —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, —C₍₀₋₃₎alkylC(O)N(R^{N1})(R^{N2}), 3- to 8-membered heterocyclyl, —C₍₁₋₃₎alkyl(3- to 8-membered heterocyclyl), or 5- to 10-membered heteroaryl, wherein the —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, 3- to 8-membered heterocyclyl, —C₍₁₋₃₎alkyl(3- to 8-membered heterocyclyl), and 5- to 10-membered heteroaryl are optionally further substituted with one to five groups selected from halo, —OH, —N(R^{N1})(R^{N2}), —C₍₁₋₄₎alkyl, —C₍₁₋₄₎haloalkyl, —OC₍₁₋₄₎alkyl, —OC₍₁₋₄₎ haloalkyl, and 3- to 5-membered heterocyclyl; or
R² and L-R³ are combined, along with the nitrogen atom to which they are attached, to form a 5- to 12-membered bicyclic ring system containing one or more heteroatoms or a 5- to 10-membered heteroaryl, each of which that is optionally further substituted with one to three groups selected from —N(R^{N1})(R^{N2}) and —C₍₁₋₆₎alkyl;
R⁴ is hydrogen, halo, or —C₍₁₋₄₎alkyl;
R⁵ is hydrogen, halo, or —C₍₁₋₄₎alkyl;
R^{N1} and R^{N2} are each independently for each occurrence hydrogen or —C₍₁₋₃₎alkyl R^{N3} is hydrogen or —C₍₁₋₄₎alkyl; and
R^{N4} is hydrogen —C₍₁₋₄₎alkyl or phenyl; or R^{N3} and R^{N4} taken together with the nitrogen atom to which they are attached form a 3- to 10-membered heterocyclyl that is optionally substituted with one to five groups selected from halo, —C₍₁₋₄₎alkyl, and —C₍₁₋₄₎haloalkyl;
wherein if R³ is —C(O)N(R^{N3})(R^{N4}), then L is not absent.

In some embodiments, disclosed herein is a compound of Formula I′, or a pharmaceutically acceptable salt thereof, wherein:
A is pyrazolyl, imidazolyl, triazolyl, thiazolyl, isoxazolyl, thiazolyl, or thiadiazolyl, wherein the pyrazolyl is optionally substituted with one —CH₃ group;
W is CH₂, CF₂, or CHR^{W}, wherein when W is CHR^{W}, then R^{W} and R¹ are taken together with the carbon atoms to which they are attached to form a C₍₃₋₅₎cycloalkyl;
X is N, C—H, or C—R^{X};
Y is N, C—H, or C—R^{Y};
R^{X} is fluorine;
R^{Y} is CH₃, —CHF₂, —CF₃, —OCH3, or —CH₂OCH₃;
R¹ is hydrogen, —CH₃, or —CF₃;
R² is hydrogen;
L is absent, —C₍₁₋₄₎alkylene, or —C₍₃₋₆₎cycloalkylene, wherein the —C₍₁₋₄₎alkylene is optionally substituted with one to three groups selected from halo, —OH, —N(R^{N1})(R^{N2}), and cyclopropyl;
R³ is —C₍₁₋₆₎alkyl, —C₍₃₋₁₀₎cycloalkyl, 3- to 10-membered heterocyclyl, a 5- to 12-membered bi- or tricyclic ring system containing one or more heteroatoms, phenyl, 5- to 10-membered heteroaryl, or —C(O)N(R^{N3})(R^{N4}); wherein: the —C₍₁₋₆₎alkyl is optionally substituted with one to five R^{3a} atoms; the —C₍₃₋₁₀₎cycloalkyl is optionally substituted with one to five R^{3b} groups; the 3- to 10-membered heterocyclyl is optionally substituted with one to five R^{3c} groups; the 5- to 12-membered bi- or tricyclic ring system containing one or more heteroatoms is optionally substituted with one to five R^{3d} groups, and the 5- to 10-membered heteroaryl is optionally substituted with one to five R^{3f} groups;
each R^{3a} independently for each occurrence is halo, —OH, or —OC₍₁₋₆₎alkyl;
each R^{3b} independently for each occurrence is halo, —OH, —C₍₁₋₆₎haloalkyl, or —OC₁₋₆₎alkyl;
each R^{3c} independently for each occurrence is halo, —C₍₁₋₆₎alkyl, —C₍₁₋₆₎haloalkyl, —C₍₃₋₈₎cycloalkyl, —OC₍₁₋₆₎alkyl, —S(O)₂C₍₁₋₄₎alkyl, or —S(O)₂C₍₃₋₈₎cycloalkyl wherein the —C₍₁₋₆₎alkyl and —OC₍₁₋₆₎alkyl are optionally further substituted with one to five groups selected from halo and —OC₍₁₋₄₎alkyl;
each R^{3d} is independently for each occurrence —C₍₁₋₆₎alkyl, —C₍₁₋₆₎haloalkyl, or —C₍₁₋₃₎alkylC₍₃₋₁₀₎cycloalkyl;
each R^{3f} is independently for each occurrence halo, —OH, —N(R^{N1})(R^{N2}), —CN, —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, —C₍₀₋₃₎alkylC(O)N(R^{N1})(R^{N2}), 3- to 8-membered heterocyclyl, —C₍₁₋₃₎alkyl(3- to 8-membered heterocyclyl), or 5- to 10-membered heteroaryl, wherein the —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, 3- to 8-membered heterocyclyl, —C₍₁₋₃₎alkyl(3- to 8-membered heterocyclyl), and 5- to 10-membered heteroaryl are optionally further substituted with one to five groups selected from halo, —OH, —N(R^{N1})(R^{N2}), —C₍₁₋₄₎alkyl, —C₍₁₋₄₎haloalkyl, —OC₍₁₋₄₎alkyl, —OC₍₁₋₄₎ haloalkyl, and 3- to 5-membered heterocyclyl; or
R² and L-R³ are combined, along with the nitrogen atom to which they are attached, to form a 5- to 12-membered bicyclic ring system containing one or more heteroatoms or a 5- to 10-membered heteroaryl, each of which that is optionally further substituted with one to three groups selected from —N(R^{N1})(R^{N2}) and —C₍₁₋₆₎alkyl;
R⁴ is hydrogen, chlorine, fluorine, or —CH₃;
R⁵ is hydrogen, chlorine, fluorine, or —CH₃;
R^{N1} and R^{N2} are each independently for each occurrence hydrogen or —C₍₁₋₃₎alkyl R^{N3} is hydrogen or —C₍₁₋₄₎alkyl; and
R^{N4} is hydrogen —C₍₁₋₄₎alkyl or phenyl; or R^{N3} and R^{N4} taken together with the nitrogen atom to which they are attached form a 3- to 10-membered heterocyclyl that is optionally substituted with one to five groups selected from halo, —C₍₁₋₄₎alkyl, and —C₍₁₋₄₎haloalkyl;
wherein if R³ is —C(O)N(R^{N3})(R^{N4}), then L is not absent.

In some embodiments, disclosed herein is a compound of Formula I′, or a pharmaceutically acceptable salt thereof, having a structure according to Formula I:

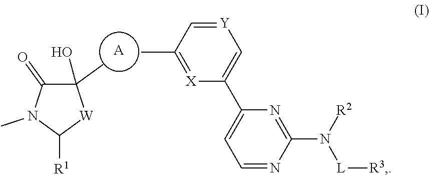

The present application further discloses a compound of Formula I:

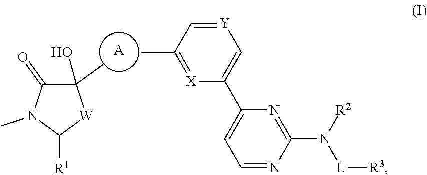

or a pharmaceutically acceptable salt thereof, wherein:
A is a 5 membered heteroaryl that is optionally substituted with one or more —C₍₁₋₄₎alkyl group;
W is CH₂, CHF, or CF₂;
X is N or C—H;
Y is N, C—H, or C—R^{Y};
R^{Y} is halo, —C₍₁₋₄₎alkyl, —C₍₁₋₄₎haloalkyl, —OC₍₁₋₄₎alkyl, or —OC₍₁₋₄₎ haloalkyl;
R¹ is hydrogen, —C₍₁₋₄₎alkyl, or —C₍₁₋₄₎haloalkyl;
R² is hydrogen or —C₍₁₋₄₎alkyl;
L is absent, —C₍₁₋₄₎alkylene, or —C₍₃₋₆₎cycloalkylene, wherein the —C₍₁₋₄₎alkylene and —C₍₃₋₆₎cycloalkylene are optionally substituted with one to three groups selected from halo, —C₍₁₋₃₎alkyl, and —OC₍₁₋₃₎alkyl;
R³ is —C₍₁₋₆₎alkyl, —C₍₃₋₁₀₎cycloalkyl, 3- to 10-membered heterocyclyl, a 5- to 12-membered bi- or tricyclic ring system containing one or more heteroatoms, —C₍₆₋₁₀₎aryl, or 5- to 10-membered heteroaryl, each of which is optionally substituted with one to five R^{3x} groups;
each R^{3x} independently for each occurrence is halo, —OH, —N(R^{N1})(R^{N2}), —CN, —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, —C₍₁₋₃₎alkylC₍₃₋₁₀₎cycloalkyl, —C₍₃₋₁₀₎cycloalkylC₍₁₋₃₎alkyl, —C(O)C₍₁₋₄₎alkyl, —S(O)₂C₍₁₋₄₎alkyl, —N(H)S(O)₂C₍₁₋₄₎alkyl, —C₍₀₋₃₎alkylC(O)N(R^{N1})(R^{N2}), 3- to 8-membered heterocyclyl, —C₍₁₋₃₎alkyl(3- to 8-membered heterocyclyl), —C₍₆₋₁₀₎aryl, or 5- to 10-membered heteroaryl, wherein the —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, —C₍₁₋₃₎alkylC₍₃₋₁₀₎cycloalkyl, —C₍₃₋₁₀₎cycloalkylC₍₁₋₃₎alkyl, —C(O)C₍₁₋₄₎alkyl, —S(O)₂C₍₁₋₄₎alkyl, —N(H)S(O)₂C₍₁₋₄₎alkyl, 3- to 8-membered heterocyclyl, —C₍₁₋₃₎alkyl(3- to 8-membered heterocyclyl), —C₍₆₋₁₀₎aryl, and 5- to 10-membered heteroaryl are optionally further substituted with one to five groups selected from halo, —OH, —N(R^{N1})(R^{N2}), —CN, —C₍₁₋₄₎alkyl, —C₍₁₋₄₎haloalkyl, —OC₍₁₋₄₎alkyl, and —OC₍₁₋₄₎ haloalkyl; or
R² and L-R³ are combined, along with the nitrogen atom to which they are attached, to form a 3- to 10-membered heterocyclyl, a 5- to 12-membered bicyclic ring system containing one or more heteroatoms, or a 5- to 10-membered heteroaryl, each of which is optionally substituted with one to three groups selected from halo, —OH, —N(R^{N1})(R^{N2}), —CN, —C₍₁₋₆₎alkyl, —C₍₁₋₆₎haloalkyl, —OC₍₁₋₆₎alkyl, and —OC₍₁₋₆₎haloalkyl; and R^{N1} and R^{N2} are each independently for each occurrence hydrogen, —C₍₁₋₃₎alkyl, or —C₍₁₋₃₎haloalkyl.

In some embodiments, disclosed herein is a compound of Formula I, or a pharmaceutically acceptable salt thereof, wherein:
A is a 5-membered heteroaryl that is optionally substituted with one —C₍₁₋₄₎alkyl group;
W is CH₂ or CF₂;
X is N or C—H;
Y is N, C—H, or C—R^{Y};
R^{Y} is —C₍₁₋₄₎alkyl or —C₍₁₋₄₎haloalkyl;
R¹ is hydrogen, —C₍₁₋₄₎alkyl, or —C₍₁₋₄₎haloalkyl;
R² is hydrogen;
L is absent, —C₍₁₋₄₎alkylene, or —C₍₃₋₆₎cycloalkylene, wherein the —C₍₁₋₄₎alkylene and —C₍₃₋₆₎cycloalkylene are optionally substituted with one to three groups selected from halo, —C₍₁₋₃₎alkyl, and —OC₍₁₋₃₎alkyl;
R³ is —C₍₁₋₆₎alkyl, —C₍₃₋₁₀₎cycloalkyl, 3- to 10-membered heterocyclyl, a 5- to 12-membered bi- or tricyclic ring system containing one or more heteroatoms, —C₍₆₋₁₀₎aryl, or 5- to 10-membered heteroaryl, each of which is optionally substituted with one to five R^{3x} groups;
each R^{3x} independently for each occurrence is halo, —OH, —N(R^{N1})(R^{N2}), —CN, —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, —C₍₁₋₃₎alkylC₍₃₋₁₀₎cycloalkyl, —C₍₀₋₃₎alkylC(O)N(R^{N1})(R^{N2}), 3- to 8-membered heterocyclyl, —C₍₁₋₃₎alkyl(3- to 8-membered heterocyclyl), —C₍₆₋₁₀₎aryl, or 5- to 10-membered heteroaryl, wherein the —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, —C₍₁₋₃₎alkylC₍₃₋₁₀₎cycloalkyl, —C(O)C₍₁₋₄₎alkyl, —S(O)₂C₍₁₋₄₎alkyl, —N(H)S(O)₂C₍₁₋₄₎alkyl, 3- to 8-membered heterocyclyl, —C₍₁₋₃₎alkyl(3- to 8-membered heterocyclyl), —C₍₆₋₁₀₎aryl, and 5- to 10-membered heteroaryl are optionally further substituted with one to five groups selected from halo, —OH, —N(R^{N1})(R^{N2}), —CN, —C₍₁₋₄₎alkyl, —C₍₁₋₄₎haloalkyl, —OC₍₁₋₄₎alkyl, and —OC₍₁₋₄₎ haloalkyl; or
R² and L-R³ are combined, along with the nitrogen atom to which they are attached, to form a 5- to 12-membered bicyclic ring system containing one or more heteroatoms or a 5- to 10-membered heteroaryl, each of which is optionally substituted with one to three groups selected from halo, —OH, —N(R^{N1})(R^{N2}), —CN, —C₍₁₋₆₎alkyl, —C₍₁₋₆₎haloalkyl, —OC₍₁₋₆₎alkyl, and —OC₍₁₋₆₎haloalkyl; and
R^{N1} and R^{N2} are each independently for each occurrence hydrogen or —C₍₁₋₃₎alkyl, or —C₍₁₋₃₎haloalkyl.

In some embodiments, disclosed herein is a compound of Formula I, or a pharmaceutically acceptable salt thereof, wherein:
A is a 5-membered heteroaryl;
W is CH₂ or CF₂;
X is N or C—H;
Y is N, C—H, or C—R^{Y};
R^{Y} is —C₍₁₋₄₎alkyl or —C₍₁₋₄₎haloalkyl;
R¹ is hydrogen, —C₍₁₋₄₎alkyl, or —C₍₁₋₄₎haloalkyl;
R² is hydrogen;
L is absent, —C₍₁₋₄₎alkylene, or —C₍₃₋₆₎cycloalkylene;
R³ is —C₍₁₋₆₎alkyl, —C₍₃₋₁₀₎cycloalkyl, 3- to 10-membered heterocyclyl, a 5- to 12-membered bi- or tricyclic ring system containing one or more heteroatoms, —C₍₆₋₁₀₎aryl, or 5- to 10-membered heteroaryl, each of which is optionally substituted with one to five R^{3x} groups;
each R^{3x} independently for each occurrence is halo, —OH, —N(R^{N1})(R^{N2}), —CN, —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, 3- to 8-membered heterocyclyl, —C₍₁₋₃₎alkyl(3- to 8-membered heterocyclyl), or 5- to 10-membered heteroaryl, wherein the —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, —C(O)C₍₁₋₄₎alkyl, —S(O)₂C₍₁₋₄₎alkyl, —N(H)S(O)₂C₍₁₋₄₎alkyl, 3- to 8-membered heterocyclyl, —C₍₁₋₃₎alkyl(3- to 8-membered heterocyclyl), and 5- to 10-membered heteroaryl are optionally further substituted with one to five groups selected from halo, —OH, —C₍₁₋₄₎alkyl, —C₍₁₋₄₎haloalkyl, —OC₍₁₋₄₎alkyl, and —OC₍₁₋₄₎ haloalkyl; or
R² and L-R³ are combined, along with the nitrogen atom to which they are attached, to form a 5- to 12-membered bicyclic ring system containing one or more heteroatoms or a 5- to 10-membered heteroaryl, each of which is optionally substituted with one to three groups selected from —N(R^{N1})(R^{N2}) and —C₍₁₋₆₎alkyl; and
R^{N1} and R^{N2} are each independently for each occurrence hydrogen or —C₍₁₋₃₎alkyl.

In some embodiments, disclosed herein is a compound of Formula I, or a pharmaceutically acceptable salt thereof, wherein:
A is pyrazolyl, triazolyl, thiazolyl, or isoxazolyl;
W is CH₂ or CF₂;
X is N or C—H;
Y is C—H or C—R^{Y};
R^{Y} is —CF₃;
R¹ is hydrogen or —CF₃;
R² is hydrogen;
L is absent, —C₍₁₋₄₎alkylene, or —C₍₃₋₆₎cycloalkylene;
R³ is —C₍₁₋₆₎alkyl, —C₍₃₋₁₀₎cycloalkyl, 3- to 10-membered heterocyclyl, a 5- to 12-membered bi- or tricyclic ring system containing one or more heteroatoms, —C₍₆₋₁₀₎aryl, or 5- to 10-membered heteroaryl, each of which is optionally substituted with one to five R^{3x} groups;
each R^{3x} independently for each occurrence is halo, —OH, —N(R^{N1})(R^{N2}), —CN, —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, 3- to 8-membered heterocyclyl, —C₍₁₋₃₎alkyl(3- to 8-membered heterocyclyl), or 5- to 10-membered heteroaryl, wherein the —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, —C(O)C₍₁₋₄₎alkyl, —S(O)₂C₍₁₋₄₎alkyl, —N(H)S(O)₂C₍₁₋₄₎alkyl, 3- to 8-membered heterocyclyl, —C₍₁₋₃₎alkyl(3- to 8-membered heterocyclyl), and 5- to 10-membered heteroaryl are optionally further substituted with one to five groups selected from halo, —OH, —C₍₁₋₄₎alkyl, —C₍₁₋₄₎haloalkyl, —OC₍₁₋₄₎alkyl, and —OC₍₁₋₄₎ haloalkyl; or
R² and L-R³ are combined, along with the nitrogen atom to which they are attached, to form a 5,6-fused bicyclic ring system containing one or more heteroatoms or a 5-membered heteroaryl, each of which is optionally substituted with one to three groups selected from —N(R^{N1})(R^{N2}) and —C₍₁₋₆₎alkyl; and
R^{N1} and R^{N2} are each independently for each occurrence hydrogen or —C₍₁₋₃₎alkyl.

In some embodiments, disclosed herein is a compound of Formula I, or a pharmaceutically acceptable salt thereof, wherein:
A is a 5-membered heteroaryl that is optionally substituted with one or more —C₍₁₋₄₎alkyl groups;
W is CH₂, CHF, or CF₂;
X is N or C—H;
Y is N, C—H, or C—R^{Y};
R^{Y} is halo, —C₍₁₋₄₎alkyl, —C₍₁₋₄₎haloalkyl, —OC₍₁₋₄₎alkyl, or —OC₍₁₋₄₎ haloalkyl;
R¹ is hydrogen, —C₍₁₋₄₎alkyl, or —C₍₁₋₄₎haloalkyl;
R² is hydrogen or —C₍₁₋₄₎alkyl;
L is absent, —C₍₁₋₄₎alkylene, or —C₍₃₋₆₎cycloalkylene, wherein the —C₍₁₋₄₎alkylene and —C₍₃₋₆₎cycloalkylene are optionally substituted with one to three groups selected from halo, —C₍₁₋₃₎alkyl, and —OC₍₁₋₃₎alkyl;
R³ is —C₍₁₋₆₎alkyl, —C₍₃₋₁₀₎cycloalkyl, 3- to 10-membered heterocyclyl, a 5- to 12-membered bi- or tricyclic ring system containing one or more heteroatoms, —C₍₆₋₁₀₎aryl, or 5- to 10-membered heteroaryl, wherein: the —C₍₁₋₆₎alkyl is optionally substituted with one to five R^{3a} groups; the —C₍₃₋₁₀₎cycloalkyl is optionally substituted with one to five R^{3b} groups; the 3- to 10-membered heterocyclyl is optionally substituted with one to five R^{3c} groups; the 5- to 12-membered bi- or tricyclic ring system containing one or more heteroatoms is optionally substituted with one to five R^{3d}; the —C₍₆₋₁₀₎aryl is optionally substituted with one to five R^{3e} groups; and the 5- to 10-membered heteroaryl is optionally substituted with one to five R^{3f} groups;
R^{3a}, R^{3b}, R^{3c}, R^{3d}, R^{3e}, and R^{3f}, are each independently for each occurrence halo, —OH, —N(R^{N1})(R^{N2}), —CN, —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, —C₍₁₋₃₎alkylC₍₃₋₁₀₎cycloalkyl, —C₍₃₋₁₀₎cycloalkylC₍₁₋₃₎alkyl, —C(O)C₍₁₋₄₎alkyl, —S(O)₂C₍₁₋₄₎alkyl, —N(H)S(O)₂C₍₁₋₄₎alkyl, —C₍₀₋₃₎alkylC(O)N(R^{N1})(R^{N2}), 3- to 8-membered heterocyclyl, —C₍₁₋₃₎alkyl(3- to 8-membered heterocyclyl), —C₍₆₋₁₀₎aryl, or 5- to 10-membered heteroaryl, wherein the —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, —C₍₁₋₃₎alkylC₍₃₋₁₀₎cycloalkyl, —C₍₃₋₁₀₎cycloalkylC₍₁₋₃₎alkyl, —C(O)C₍₁₋₄₎alkyl, —S(O)₂C₍₁₋₄₎alkyl, —N(H)S(O)₂C₍₁₋₄₎alkyl, 3- to 8-membered heterocyclyl, —C₍₁₋₃₎alkyl(3- to 8-membered heterocyclyl), —C₍₆₋₁₀₎aryl, and 5- to 10-membered heteroaryl are optionally further substituted with one to five groups selected from halo, —OH, —N(R^{N1})(R^{N2}), —CN, —C₍₁₋₄₎alkyl, —C₍₁₋₄₎haloalkyl, —OC₍₁₋₄₎alkyl, and —OC₍₁₋₄₎haloalkyl; or
R² and L-R³ are combined, along with the nitrogen atom to which they are attached, to form a 3- to 10-membered heterocyclyl, a 5- to 12-membered bicyclic ring system containing one or more heteroatoms, or a 5- to 10-membered heteroaryl, each of which is optionally substituted with one to three groups selected from halo, —OH, —N(R^{N1})(R^{N2}), —CN, —C₍₁₋₆₎alkyl, —C₍₁₋₆₎haloalkyl, —OC₍₁₋₆₎alkyl, and —OC₍₁₋₆₎haloalkyl; and
R^{N1} and R^{N2} are each independently for each occurrence hydrogen, —C₍₁₋₃₎alkyl, or —C₍₁₋₃₎haloalkyl.

In some embodiments, disclosed herein is a compound of Formula I, or a pharmaceutically acceptable salt thereof, wherein:
A is a 5-membered heteroaryl that is optionally substituted with one —C₍₁₋₄₎alkyl group;
W is CH₂ or CF₂;
X is N or C—H;
Y is N, C—H, or C—R^{Y};
R^{Y} is halo, —C₍₁₋₄₎alkyl, —C₍₁₋₄₎haloalkyl, —OC₍₁₋₄₎alkyl, or —OC₍₁₋₄₎ haloalkyl;
R¹ is hydrogen, —C₍₁₋₄₎alkyl, or —C₍₁₋₄₎haloalkyl;
R² is hydrogen;
L is absent, —C₍₁₋₄₎alkylene, or —C₍₃₋₆₎cycloalkylene, wherein the —C₍₁₋₄₎alkylene and —C₍₃₋₆₎cycloalkylene are optionally substituted with one to three groups selected from halo, —C₍₁₋₃₎alkyl, and —OC₍₁₋₃₎alkyl;
R³ is —C₍₁₋₆₎alkyl, —C₍₃₋₁₀₎cycloalkyl, 3- to 10-membered heterocyclyl, a 5- to 12-membered bi- or tricyclic ring system containing one or more heteroatoms, —C₍₆₋₁₀₎aryl, or 5- to 10-membered heteroaryl, wherein: the —C₍₁₋₆₎alkyl is optionally substituted with one to five R^{3a} groups; the —C₍₃₋₁₀₎cycloalkyl is optionally substituted with one to five R^{3b} groups; the 3- to 10-membered heterocyclyl is optionally substituted with one to five R^{3e} groups; the 5- to 12-membered bi- or tricyclic ring system containing one or more heteroatoms is optionally substituted with one to five R^{3d}; the —C₍₆₋₁₀₎aryl is optionally substituted with one to five R^{3e} groups; and the 5- to 10-membered heteroaryl is optionally substituted with one to five R^{3f} groups;
each R^{3a} independently for each occurrence is halo, —OH, —N(R^{N1})(R^{N2}), —CN, —C₍₃₋₈₎cycloalkyl, —OC₍₁₋₆₎alkyl, —OC₍₁₋₆₎haloalkyl, —OC₍₃₋₈₎cycloalkyl, —S(O)₂C₍₁₋₄₎alkyl, —N(H)S(O)₂C₍₁₋₄₎alkyl, or 3- to 8-membered heterocyclyl, wherein the —C₍₃₋₈₎cycloalkyl, —OC₍₁₋₆₎alkyl, —OC₍₁₋₆₎haloalkyl, —OC₍₃₋₈₎cycloalkyl, and 3- to 8-membered heterocyclyl are optionally further substituted with one to five groups selected from halo, —OH, —N(R^{N1})(R^{N2}), —CN, —C₍₁₋₄₎alkyl, —C₍₁₋₄₎haloalkyl, —OC₍₁₋₄₎alkyl, and —OC₍₁₋₄₎ haloalkyl;
R^{3b}, R^{3c}, and R^{3d} are each independently for each occurrence halo, —OH, —N(R^{N1})(R^{N2}), —CN, —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —C(O)C₍₁₋₄₎alkyl, —S(O)₂C₍₁₋₄₎alkyl, —N(H)S(O)₂C₍₁₋₄₎alkyl, —C₍₀₋₃₎alkylC(O)N(R^{N1})(R^{N2}), 3- to 8-membered heterocyclyl, or 5- to 10-membered heteroaryl, wherein the —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, 3- to 8-membered heterocyclyl, and 5- to 10-membered heteroaryl are optionally further substituted with one to five groups selected from halo, —OH, —N(R^{N1})(R^{N2}), —CN, —C₍₁₋₄₎alkyl, —C₍₁₋₄₎haloalkyl, —OC₍₁₋₄₎alkyl, and —OC₍₁₋₄₎ haloalkyl;
R^{3e} e and R^{3f} are each independently for each occurrence halo, —OH, —N(R^{N1})(R^{N2}), —CN, —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, —C₍₁₋₃₎alkylC₍₃₋₁₀₎cycloalkyl, —C(O)C₍₁₋₄₎alkyl, —S(O)₂C₍₁₋₄₎alkyl, —N(H)S(O)₂C₍₁₋₄₎alkyl, —C₍₀₋₃₎alkylC(O)N(R^{N1})(R^{N2}), 3- to 8-membered heterocyclyl, —C₍₁₋₃₎alkyl(3- to 8-membered heterocyclyl), —C₍₆₋₁₀₎aryl, or 5- to 10-membered heteroaryl, wherein the —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, —C₍₁₋₃₎alkylC₍₃₋₁₀₎cycloalkyl, 3- to 8-membered heterocyclyl, —C₍₁₋₃₎alkyl(3- to 8-membered heterocyclyl), —C₍₆₋₁₀₎aryl, and 5- to 10-membered heteroaryl are optionally further substituted with one to five groups selected from halo, —OH, —N(R^{N1})(R^{N2}), —CN, —C₍₁₋₄₎alkyl, —C₍₁₋₄₎haloalkyl, —OC₍₁₋₄₎alkyl, and —OC₍₁₋₄₎ haloalkyl; or
R² and L-R³ are combined, along with the nitrogen atom to which they are attached, to form a 3- to 10-membered heterocyclyl, a 5- to 12-membered bicyclic ring system containing one or more heteroatoms, or a 5- to 10-membered heteroaryl, each of which is optionally substituted with one to three groups selected from halo, —OH, —N(R^{N1})(R^{N2}), —CN, —C₍₁₋₆₎alkyl, —C₍₁₋₆₎haloalkyl, —OC₍₁₋₆₎alkyl, and —OC₍₁₋₆₎haloalkyl; and
R^{N1} and R^{N2} are each independently for each occurrence hydrogen or —C₍₁₋₃₎alkyl.

In some embodiments, disclosed herein is a compound of Formula I, or a pharmaceutically acceptable salt thereof, wherein:
A is a 5-membered heteroaryl that is optionally substituted with one —C₍₁₋₄₎alkyl group;
W is CH₂ or CF₂;
X is N or C—H;
Y is N, C—H, or C—R^{Y};
R^{Y} is —C₍₁₋₄₎alkyl, —C₍₁₋₄₎haloalkyl, or —OC₍₁₋₄₎alkyl;
R¹ is hydrogen, —C₍₁₋₄₎alkyl, or —C₍₁₋₄₎haloalkyl;
R² is hydrogen;
L is absent, —C₍₁₋₄₎alkylene, or —C₍₃₋₆₎cycloalkylene, wherein the —C₍₁₋₄₎alkylene and —C₍₃₋₆₎cycloalkylene are optionally substituted with one to three groups selected from halo, —C₍₁₋₃₎alkyl, and —OC₍₁₋₃₎alkyl;
R³ is —C₍₁₋₆₎alkyl, —C₍₃₋₁₀₎cycloalkyl, 3- to 10-membered heterocyclyl, a 5- to 12-membered bi- or tricyclic ring system containing one or more heteroatoms, or 5- to 10-membered heteroaryl, wherein: the —C₍₁₋₆₎alkyl is optionally substituted with one to five R^{3a} groups; the —C₍₃₋₁₀₎cycloalkyl is optionally substituted with one to five R^{3b} groups; the 3- to 10-membered heterocyclyl is optionally substituted with one to five R^{3e} groups; the 5- to 12-membered bi- or tricyclic ring system containing one or more heteroatoms is optionally substituted with one to five R^{3d}; and the 5- to 10-membered heteroaryl is optionally substituted with one to five R^{3f} groups;
each R^{3a} independently for each occurrence is halo, —OH, —N(R^{N1})(R^{N2}), or —CN;
R^{3b}, R^{3c}, and R^{3d} are each independently for each occurrence halo, —OH, —N(R^{N1})(R^{N2}), —CN, —C₍₁₋₆₎alkyl, —C₍₁₋₆₎haloalkyl, —OC₍₁₋₆₎alkyl, or —OC₍₁₋₆₎haloalkyl, wherein the —C₍₁₋₆₎alkyl, —C₍₁₋₆₎haloalkyl, —OC₍₁₋₆₎alkyl, and —OC₍₁₋₆₎haloalkyl are optionally further substituted with one to five groups selected from halo, —OH, —N(R^{N1})(R^{N2}), and —CN;
each R^{3f} is independently for each occurrence halo, —OH, —N(R^{N1})(R^{N2}), —CN, —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, —C₍₁₋₃₎alkylC₍₃₋₁₀₎cycloalkyl, —C₍₀₋₃₎alkylC(O)N(R^{N1})(R^{N2}), 3- to 8-membered heterocyclyl, —C₍₁₋₃₎alkyl(3- to 8-membered heterocyclyl), or —C₍₆₋₁₀₎aryl, wherein the —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, —C₍₁₋₃₎alkylC₍₃₋₁₀₎cycloalkyl, 3- to 8-membered heterocyclyl, —C₍₁₋₃₎alkyl(3- to 8-membered heterocyclyl), and —C₍₆₋₁₀₎aryl are optionally further substituted with one to five groups selected from halo, —OH, —N(R^{N1})(R^{N2}), —CN, —C₍₁₋₄₎alkyl, —C₍₁₋₄₎haloalkyl, —OC₍₁₋₄₎alkyl, and —OC₍₁₋₄₎ haloalkyl; or
R² and L-R³ are combined, along with the nitrogen atom to which they are attached, to form a 5- to 12-membered bicyclic ring system containing one or more heteroatoms or a 5- to 10-membered heteroaryl, each of which is optionally substituted with one to three groups selected from halo, —OH, —N(R^{N1})(R^{N2}), —CN, —C₍₁₋₆₎alkyl, —C₍₁₋₆₎haloalkyl, —OC₍₁₋₆₎alkyl and —OC₍₁₋₆₎haloalkyl; and
R^{N1} and R^{N2} are each independently for each occurrence hydrogen or —C₍₁₋₃₎alkyl.

In some embodiments, disclosed herein is a compound of Formula I, or a pharmaceutically acceptable salt thereof, wherein:
A is a 5-membered heteroaryl;
W is CH₂ or CF₂;
X is N or C—H;
Y is N, C—H, or C—R^{Y};
R^{Y} is —C₍₁₋₄₎alkyl, —C₍₁₋₄₎haloalkyl, or —OC₍₁₋₄₎alkyl;
R¹ is hydrogen, —C₍₁₋₄₎alkyl, or —C₍₁₋₄₎haloalkyl;
R² is hydrogen;
L is absent, —C₍₁₋₄₎alkylene, or —C₍₃₋₆₎cycloalkylene;
R³ is —C₍₁₋₆₎alkyl, —C₍₃₋₁₀₎cycloalkyl, 3- to 10-membered heterocyclyl, a 5- to 12-membered bi- or tricyclic ring system containing one or more heteroatoms, or 5- to 10-membered heteroaryl; wherein: the —C₍₁₋₆₎alkyl is optionally substituted with one to five halo groups; the —C₍₃₋₁₀₎cycloalkyl is optionally substituted with one to five —OH groups; the 5- to 12-membered bi- or tricyclic ring system containing one or more heteroatoms is optionally substituted with one to five R^{3d}; and the 5- to 10-membered heteroaryl is optionally substituted with one to five R^{3f} groups;
each R^{3d} is independently for each occurrence halo, —OH, —N(R^{N1})(R^{N2}), —CN, —C₍₁₋₆₎alkyl, —C₍₁₋₆₎haloalkyl, —OC₍₁₋₆₎alkyl, or —OC₍₁₋₆₎haloalkyl;
each R^{3f} is independently for each occurrence halo, —OH, —N(R^{N1})(R^{N2}), —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, 3- to 8-membered heterocyclyl, or —C₍₁₋₃₎alkyl(3- to 8-membered heterocyclyl), wherein the —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, 3- to 8-membered heterocyclyl, and —C₍₁₋₃₎alkyl(3- to 8-membered heterocyclyl) are optionally further substituted with one to five groups selected from halo, —OH, —C₍₁₋₄₎alkyl, —C₍₁₋₄₎haloalkyl, —OC₍₁₋₄₎alkyl, and —OC₍₁₋₄₎ haloalkyl; or
R² and L-R³ are combined, along with the nitrogen atom to which they are attached, to form a 5- to 12-membered bicyclic ring system containing one or more heteroatoms or a 5- to 10-membered heteroaryl, each of which is optionally substituted with one to three groups selected from —N(R^{N1})(R^{N2}) and —C₍₁₋₆₎alkyl; and
R^{N1} and R^{N2} are each independently for each occurrence hydrogen or —C₍₁₋₃₎alkyl.

In some embodiments, disclosed herein is a compound of Formula I, or a pharmaceutically acceptable salt thereof, wherein:
A is pyrazolyl, triazolyl, thiazolyl, or isoxazolyl;
W is CH₂ or CF₂;
X is N or C—H;
Y is C—H or C—R^{Y};
R^{Y} is —CF₃;
R¹ is hydrogen or —CF₃;
R² is hydrogen;
L is absent, —C₍₁₋₄₎alkylene, or —C₍₃₋₆₎cycloalkylene;
R³ is —C₍₁₋₆₎alkyl, —C₍₃₋₁₀₎cycloalkyl, 3- to 10-membered heterocyclyl, a 5- to 12-membered bi- or tricyclic ring system containing one or more heteroatoms, or 5- to 10-membered heteroaryl; wherein: the —C₍₁₋₆₎alkyl is optionally substituted with one to five fluorine atoms; the —C₍₃₋₁₀₎cycloalkyl is optionally substituted with one —OH group; the a 5- to 12-membered bi- or tricyclic ring system containing one or more heteroatoms is optionally substituted with one to five —C₍₁₋₆₎alkyl groups, and the 5- to 10-membered heteroaryl is optionally substituted with one to five R^{3f} groups;
each R^{3f} is independently for each occurrence —N(R^{N1})(R^{N2}), —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, 3- to 8-membered heterocyclyl, or —C₍₁₋₃₎alkyl(3- to 8-membered heterocyclyl), wherein the —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, 3- to 8-membered heterocyclyl, and —C₍₁₋₃₎alkyl(3- to 8-membered heterocyclyl) are optionally further substituted with one to five groups selected from halo, —OH, —C₍₁₋₄₎alkyl, —C₍₁₋₄₎haloalkyl, —OC₍₁₋₄₎alkyl, and —OC₍₁₋₄₎ haloalkyl; or
R² and L-R³ are combined, along with the nitrogen atom to which they are attached, to form a 5- to 12-membered bicyclic ring system containing one or more heteroatoms or a 5- to 10-membered heteroaryl, each of which is optionally substituted with one to three groups selected from —N(R^{N1})(R^{N2}) and —C₍₁₋₆₎alkyl, and
R^{N1} and R^{N2} are each independently for each occurrence hydrogen or —C₍₁₋₃₎alkyl.

In some embodiments, disclosed herein is a compound of Formula I′, or a pharmaceutically acceptable salt thereof, which is a compound of Formula Ia-1′:

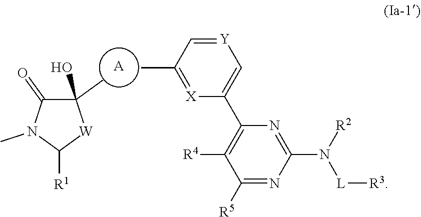

In some embodiments, disclosed herein is a compound of Formula I′, or a pharmaceutically acceptable salt thereof, which is a compound of Formula Ia-2′:

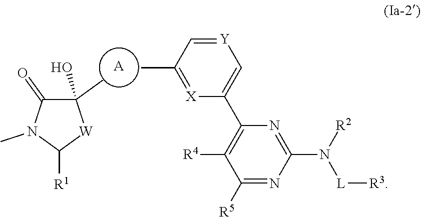

In some embodiments, disclosed herein is a compound of Formula I′ or Formula I, or a pharmaceutically acceptable salt thereof, which is a compound of Formula Ia-1:

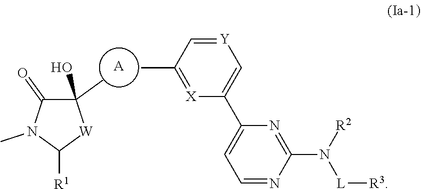

In some embodiments, disclosed herein is a compound of Formula I′ or Formula I, or a pharmaceutically acceptable salt thereof, which is a compound of Formula Ia-2:

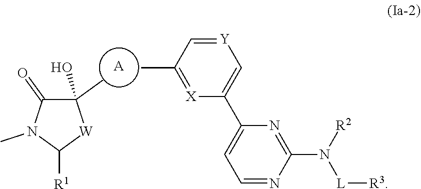

In some embodiments, disclosed herein is a compound of Formula I′ or Formula I, or a pharmaceutically acceptable salt thereof, wherein A is a 5-membered heteroaryl substituted with one —C₍₁₋₄₎alkyl group. In some embodiments, disclosed herein is a compound of Formula I′ or Formula I, or a pharmaceutically acceptable salt thereof, wherein A is a 5-membered heteroaryl. In some embodiments, disclosed herein is a compound of Formula I′ or Formula I, or a pharmaceutically acceptable salt thereof, wherein A is pyrazolyl, triazolyl, thiazolyl, or isoxazolyl. In some embodiments, disclosed herein is a compound of Formula I′ or Formula I, or a pharmaceutically acceptable salt thereof, wherein A is triazolyl or isoxazolyl. In some embodiments, disclosed herein is a compound of Formula I′ or Formula I, or a pharmaceutically acceptable salt thereof, wherein A is pyrazolyl. In some embodiments, disclosed herein is a compound of Formula I′ or Formula I, or a pharmaceutically acceptable salt thereof, wherein A is triazolyl. In some embodiments, disclosed herein is a compound of Formula I′ or Formula I, or a pharmaceutically acceptable salt thereof, wherein A is thiazolyl. In some embodiments, disclosed herein is a compound of Formula I′ or Formula I, or a pharmaceutically acceptable salt thereof, wherein A is isoxazolyl.

In some embodiments, disclosed herein is a compound of Formula I′ or Formula I, or a pharmaceutically acceptable salt thereof, wherein A is

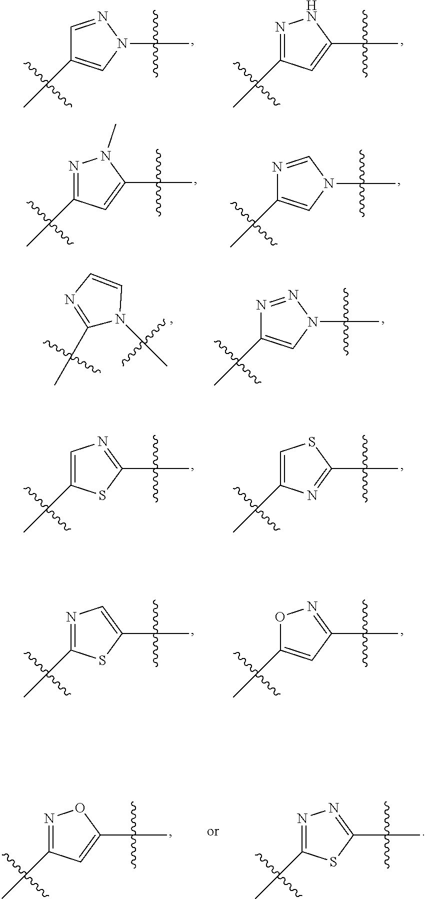

In some embodiments, disclosed herein is a compound of Formula I′ or Formula I, or a pharmaceutically acceptable salt thereof, wherein A is

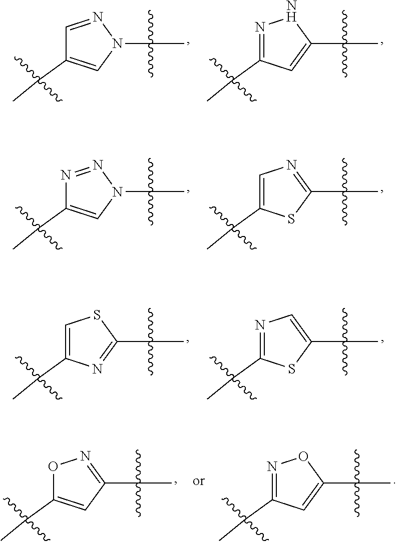

In some embodiments, disclosed herein is a compound of Formula I′ or Formula I, or a pharmaceutically acceptable salt thereof, wherein A is

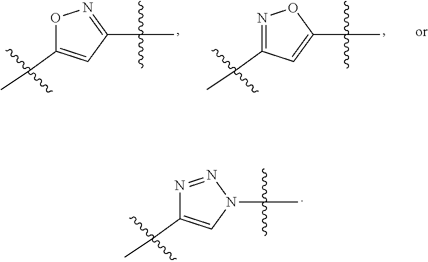 [SMILES: *c1cc(*)on1 ; *c1cc(*)on1 ; *c1cn(*)nn1]

In some embodiments, disclosed herein is a compound of Formula I′ or Formula I, or a pharmaceutically acceptable salt thereof, wherein A is

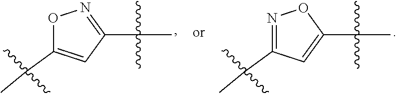 [SMILES: *c1cc(*)on1 ; *c1cc(*)on1]

In some embodiments, disclosed herein is a compound of Formula I′ or Formula I, or a pharmaceutically acceptable salt thereof, wherein A is

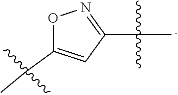 [SMILES: *c1cc(*)on1]

In some embodiments, disclosed herein is a compound of Formula I′ or Formula I, or a pharmaceutically acceptable salt thereof, wherein A is

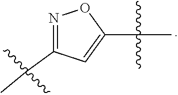 [SMILES: *c1cc(*)on1]

In some embodiments, disclosed herein is a compound of Formula I′ or Formula I, or a pharmaceutically acceptable salt thereof, wherein A is

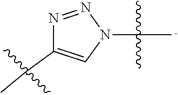 [SMILES: *c1cn(*)nn1]

In some embodiments, disclosed herein is a compound of Formula I′, or a pharmaceutically acceptable salt thereof, which is a compound of Formula Ib-1′:

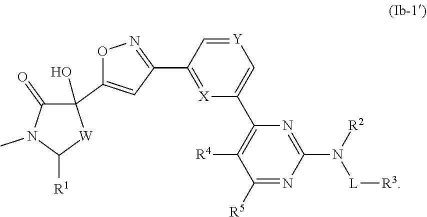

In some embodiments, disclosed herein is a compound of Formula I′, or a pharmaceutically acceptable salt thereof, which is a compound of Formula Ib-2′:

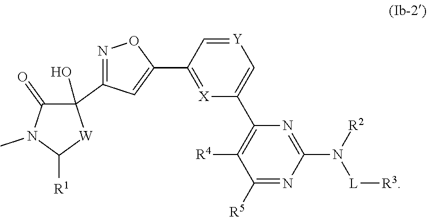

In some embodiments, disclosed herein is a compound of Formula I′, or a pharmaceutically acceptable salt thereof, which is a compound of Formula Ib-3′:

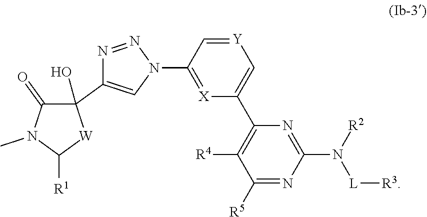

In some embodiments, disclosed herein is a compound of Formula I′ or Formula I, or a pharmaceutically acceptable salt thereof, which is a compound of Formula Ib-1:

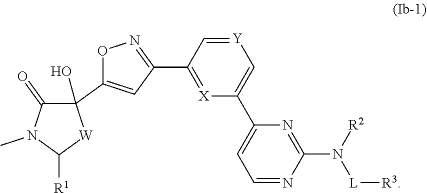

In some embodiments, disclosed herein is a compound of Formula I′ or Formula I, or a pharmaceutically acceptable salt thereof, which is a compound of Formula Ib-2:

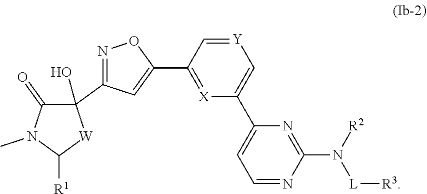

In some embodiments, disclosed herein is a compound of Formula I′ or Formula I, or a pharmaceutically acceptable salt thereof, which is a compound of Formula Ib-3:

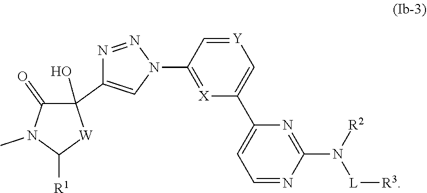

In some embodiments, disclosed herein is a compound of Formula I′, or a pharmaceutically acceptable salt thereof, wherein W is CH₂, CDH, CD₂, CF₂, or CHR^{W}. In some embodiments, disclosed herein is a compound of Formula I′ or Formula I, or a pharmaceutically acceptable salt thereof, wherein W is CH₂, CDH, CD₂, or CF₂. In some embodiments, disclosed herein is a compound of Formula I′ or Formula I, or a pharmaceutically acceptable salt thereof, wherein W is CH₂ or CF₂. In some embodiments, disclosed herein is a compound of Formula I′ or Formula I, or a pharmaceutically acceptable salt thereof, wherein W is CH₂. In some embodiments, disclosed herein is a compound of Formula I′ or Formula I, or a pharmaceutically acceptable salt thereof, wherein W is CF₂. In some embodiments, disclosed herein is a compound of Formula I′, or a pharmaceutically acceptable salt thereof, wherein W is CHR^{W}.

In some embodiments, disclosed herein is a compound of Formula I′ or Formula I, or a pharmaceutically acceptable salt thereof, wherein X is N. In some embodiments, disclosed herein is a compound of Formula I′ or Formula I, or a pharmaceutically acceptable salt thereof, wherein X is C—H. In some embodiments, disclosed herein is a compound of Formula I′, or a pharmaceutically acceptable salt thereof, wherein X is C—R^{X}. In some embodiments, disclosed herein is a compound of Formula I′, or a pharmaceutically acceptable salt thereof, wherein X is C—F. In some embodiments, disclosed herein is a compound of Formula I′, or a pharmaceutically acceptable salt thereof, wherein X is N, C—H, or C—F.

In some embodiments, disclosed herein is a compound of Formula I′ or Formula I, or a pharmaceutically acceptable salt thereof, wherein Y is N, C—H, C—CH₃, C—CHF₂, C—CF₃, C—OCH₃, or C—CH₂OCH₃. In some embodiments, disclosed herein is a compound of Formula I′ or Formula I, or a pharmaceutically acceptable salt thereof, wherein Y is N. In some embodiments, disclosed herein is a compound of Formula I′ or Formula I, or a pharmaceutically acceptable salt thereof, wherein Y is C—H or C—R^{Y}. In some embodiments, disclosed herein is a compound of Formula I′ or Formula I, or a pharmaceutically acceptable salt thereof, wherein Y is C—H or C—CF₃. In some embodiments, disclosed herein is a compound of Formula I′ or Formula I, or a pharmaceutically acceptable salt thereof, wherein Y is C—H. In some embodiments, disclosed herein is a compound of Formula I′ or Formula I, or a pharmaceutically acceptable salt thereof, wherein Y is C—R^{Y}. In some embodiments, disclosed herein is a compound of Formula I′ or Formula I, or a pharmaceutically acceptable salt thereof, wherein Y is C—CH₃. In some embodiments, disclosed herein is a compound of Formula I′ or Formula I, or a pharmaceutically acceptable salt thereof, wherein Y is C—CHF₂. In some embodiments, disclosed herein is a compound of Formula I′ or Formula I, or a pharmaceutically acceptable salt thereof, wherein Y is C—CF₃. In some embodiments, disclosed herein is a compound of Formula I′ or Formula I, or a pharmaceutically acceptable salt thereof, wherein Y is C—OCH₃. In some embodiments, disclosed herein is a compound of Formula I′, or a pharmaceutically acceptable salt thereof, wherein Y is C—CH₂OCH₃.

In some embodiments, disclosed herein is a compound of Formula I′, or a pharmaceutically acceptable salt thereof, wherein R^{W} is —C₍₁₋₄₎alkyl. In some embodiments, disclosed herein is a compound of Formula I′, or a pharmaceutically acceptable salt thereof, wherein R^{W} is —C₍₁₋₄₎haloalkyl. In some embodiments, disclosed herein is a compound of Formula I′, or a pharmaceutically acceptable salt thereof, wherein when W is CHR^{W}, then R^{W} and R¹ are taken together with the carbon atoms to which they are attached to form a C₍₃₋₅₎cycloalkyl. In some embodiments, disclosed herein is a compound of Formula I′, or a pharmaceutically acceptable salt thereof, wherein when W is CHR^{W}, then R^{W} and R¹ are taken together with the carbon atoms to which they are attached to form a C₍₃₎cycloalkyl.

In some embodiments, disclosed herein is a compound of Formula I′, or a pharmaceutically acceptable salt thereof, wherein R^{X} is halo. In some embodiments, disclosed herein is a compound of Formula I′, or a pharmaceutically acceptable salt thereof, wherein R^{X} is fluorine.

In some embodiments, disclosed herein is a compound of Formula I′ or Formula I, or a pharmaceutically acceptable salt thereof, wherein R^{Y} is —C₍₁₋₄₎alkyl, —C₍₁₋₄₎)haloalkyl, or —OC₍₁₋₄₎alkyl. In some embodiments, disclosed herein is a compound of Formula I′ or Formula I, or a pharmaceutically acceptable salt thereof, wherein R^{Y} is —C₍₁₋₄₎alkyl or —C₍₁₋₄₎haloalkyl. In some embodiments, disclosed herein is a compound of Formula I′ or Formula I, or a pharmaceutically acceptable salt thereof, wherein R^{Y} is —C₍₁₋₄₎alkyl. In some embodiments, disclosed herein is a compound of Formula I′ or Formula I, or a pharmaceutically acceptable salt thereof, wherein R^{Y} is —CH₃. In some embodiments, disclosed herein is a compound of Formula I′ or Formula I, or a pharmaceutically acceptable salt thereof, wherein R^{Y} is —C₍₁₋₄₎haloalkyl. In some embodiments, disclosed herein is a compound of Formula I′ or Formula I, or a pharmaceutically acceptable salt thereof, wherein R^{Y} is —CF₃.

In some embodiments, disclosed herein is a compound of Formula I′, or a pharmaceutically acceptable salt thereof, which is a compound of Formula Ic-1′:

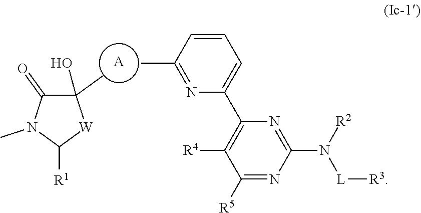

In some embodiments, disclosed herein is a compound of Formula I′, or a pharmaceutically acceptable salt thereof, which is a compound of Formula Ic-2′:

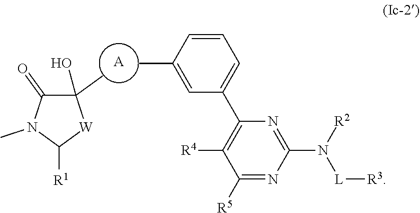

In some embodiments, disclosed herein is a compound of Formula I′ or Formula I, or a pharmaceutically acceptable salt thereof, which is a compound of Formula Ic-1:

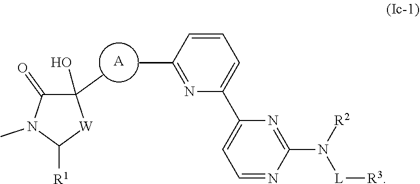

In some embodiments, disclosed herein is a compound of Formula I′ or Formula I, or a pharmaceutically acceptable salt thereof, which is a compound of Formula Ic-2:

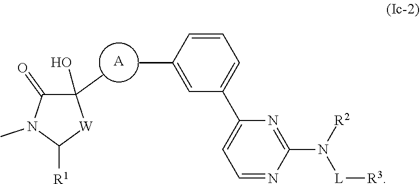

In some embodiments, disclosed herein is a compound of Formula I′ or Formula I, or a pharmaceutically acceptable salt thereof, wherein R¹ is hydrogen or —C₍₁₋₄₎haloalkyl. In some embodiments, disclosed herein is a compound of Formula I′ or Formula I, or a pharmaceutically acceptable salt thereof, wherein R¹ is hydrogen or —CF₃. In some embodiments, disclosed herein is a compound of Formula I′ or Formula I, or a pharmaceutically acceptable salt thereof, wherein R¹ is hydrogen. In some embodiments, disclosed herein is a compound of Formula I′ or Formula I, or a pharmaceutically acceptable salt thereof, wherein R¹ is —CH₃. In some embodiments, disclosed herein is a compound of Formula I′ or Formula I, or a pharmaceutically acceptable salt thereof, wherein R¹ is —CF₃. In some embodiments, disclosed herein is a compound of Formula I′, or a pharmaceutically acceptable salt thereof, wherein R^{W} and R¹ are taken together with the carbon atoms to which they are attached to form a C₍₃₋₅₎cycloalkyl. In some embodiments, disclosed herein is a compound of Formula I′, or a pharmaceutically acceptable salt thereof, wherein R^{W} and R¹ are taken together with the carbon atoms to which they are attached to form a C₍₃₎cycloalkyl.

In some embodiments, disclosed herein is a compound of Formula I′ or Formula I, or a pharmaceutically acceptable salt thereof, which is a compound of any one of Formulas Id-1 to Id-7:

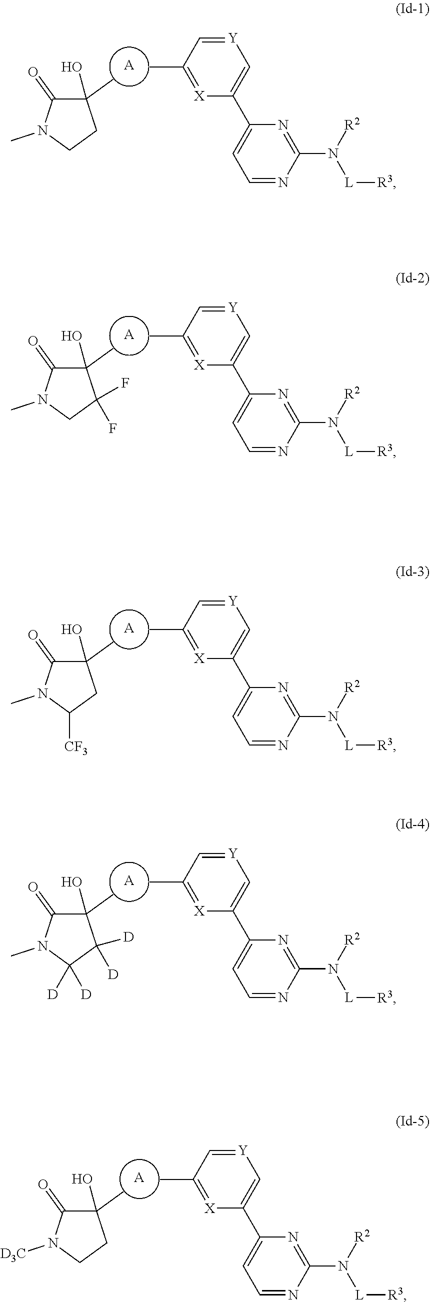

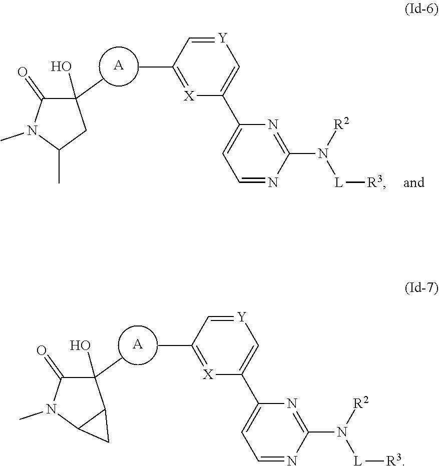

In some embodiments, disclosed herein is a compound of Formula I′ or Formula I, or a pharmaceutically acceptable salt thereof, which is a compound of any one of Formulas Id-1 to Id-5:

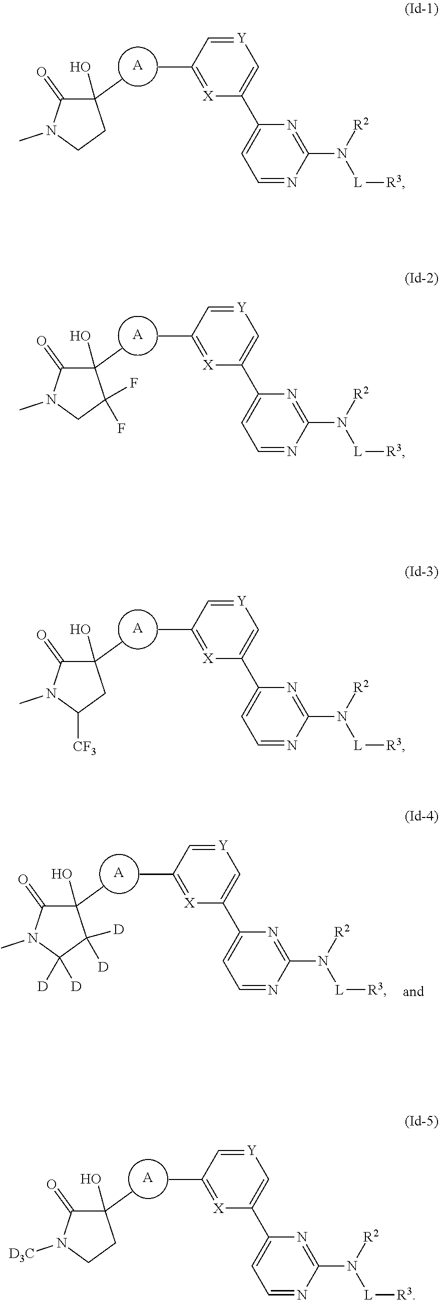

In some embodiments, disclosed herein is a compound of Formula I′ or Formula I or a pharmaceutically acceptable salt thereof, wherein R² is hydrogen.

In some embodiments, disclosed herein is a compound of Formula I′, or a pharmaceutically acceptable salt thereof, wherein L is L is absent, —C₍₁₋₄₎alkylene, or —C₍₃₋₆₎cycloalkylene, wherein the —C₍₁₋₄₎alkylene is optionally substituted with one to three groups selected from halo, —OH, —N(R^{N1})(R^{N2}), and cyclopropyl. In some embodiments, disclosed herein is a compound of Formula I′ or Formula I, or a pharmaceutically acceptable salt thereof, wherein L is absent, —C₍₁₋₄₎alkylene, or —C₍₃₋₆₎cycloalkylene, wherein the —C₍₁₋₄₎alkylene is optionally substituted with one —OC₍₁₋₃₎alkyl group. In some embodiments, disclosed herein is a compound of Formula I′ or Formula I, or a pharmaceutically acceptable salt thereof, wherein L is absent, —C₍₁₋₄₎alkylene, or —C₍₃₋₆₎cycloalkylene. In some embodiments, disclosed herein is a compound of Formula I′ or Formula I, or a pharmaceutically acceptable salt thereof, wherein L is absent, —C₍₁₋₄₎alkylene, or cyclopropylene.

In some embodiments, disclosed herein is a compound of Formula I′, or a pharmaceutically acceptable salt thereof, wherein L is absent,

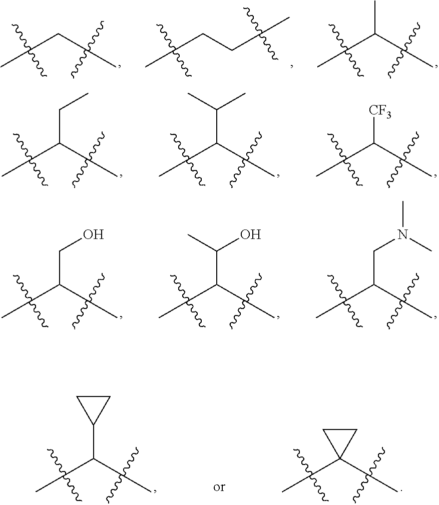

In some embodiments, disclosed herein is a compound of Formula I′ or Formula I, or a pharmaceutically acceptable salt thereof, wherein L is absent,

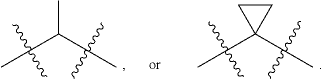

In some embodiments, disclosed herein is a compound of Formula I′ or Formula I, or a pharmaceutically acceptable salt thereof, wherein L is absent,

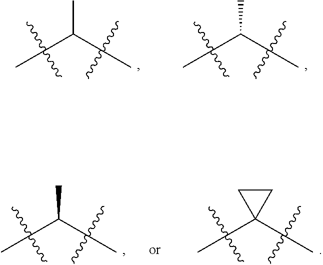

In some embodiments, disclosed herein is a compound of Formula I′ or Formula I, or a pharmaceutically acceptable salt thereof, wherein L is absent or

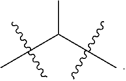 [SMILES: *C(*)C]

In some embodiments, disclosed herein is a compound of Formula I′ or Formula I, or a pharmaceutically acceptable salt thereof, wherein L is absent.

In some embodiments, disclosed herein is a compound of Formula I′ or Formula I, or a pharmaceutically acceptable salt thereof, wherein L is

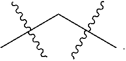 [SMILES: *C*]

In some embodiments, disclosed herein is a compound of Formula I′ or Formula I, or a pharmaceutically acceptable salt thereof, wherein L is

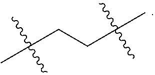 [SMILES: *CC*]

In some embodiments, disclosed herein is a compound of Formula I′ or Formula I, or a pharmaceutically acceptable salt thereof, wherein L is

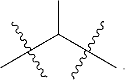 [SMILES: *C(*)C]

In some embodiments, disclosed herein is a compound of Formula I′ or Formula I, or a pharmaceutically acceptable salt thereof, wherein L is

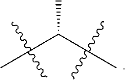

In some embodiments, disclosed herein is a compound of Formula I′ or Formula I, or a pharmaceutically acceptable salt thereof, wherein L is

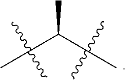

In some embodiments, disclosed herein is a compound of Formula I′ or Formula I, or a pharmaceutically acceptable salt thereof, wherein L is

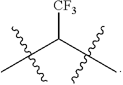 [SMILES: *C(*)C(F)(F)F]

In some embodiments, disclosed herein is a compound of Formula I′ or Formula I, or a pharmaceutically acceptable salt thereof, wherein L is

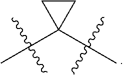 [SMILES: *C1(*)CC1]

In some embodiments, disclosed herein is a compound of Formula I′ or Formula I, or a pharmaceutically acceptable salt thereof, which is a compound of any one of Formulas Ie-1 to Ie-10:

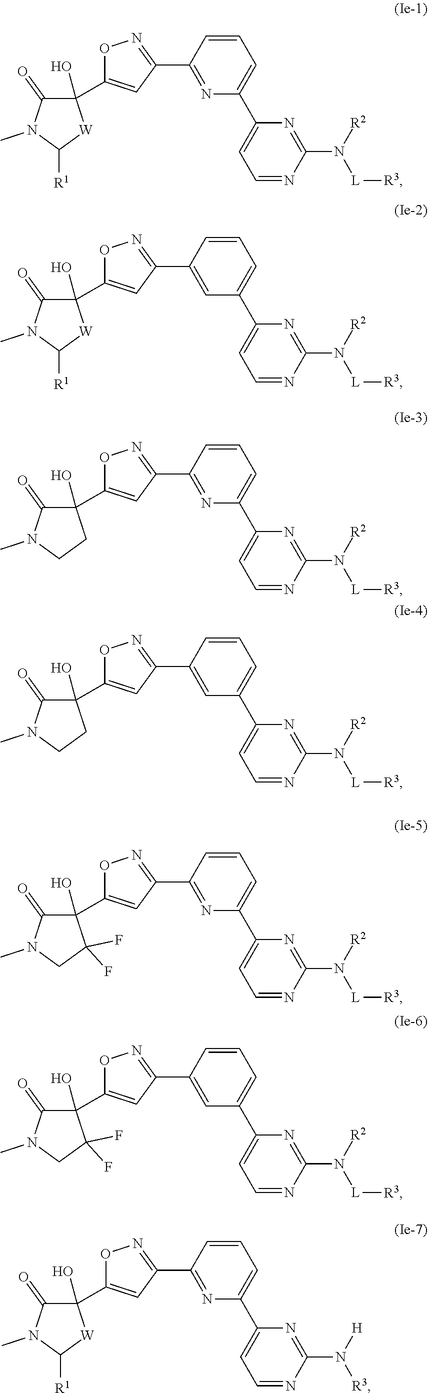

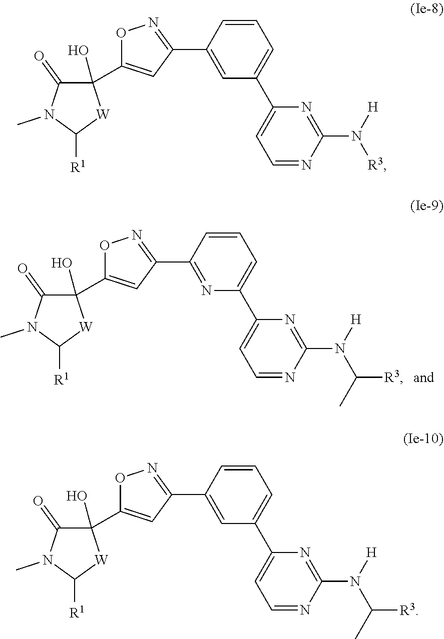

In some embodiments, disclosed herein is a compound of Formula I′ or Formula I, or a pharmaceutically acceptable salt thereof, which is a compound of any one of Formulas If-1 to If-10:

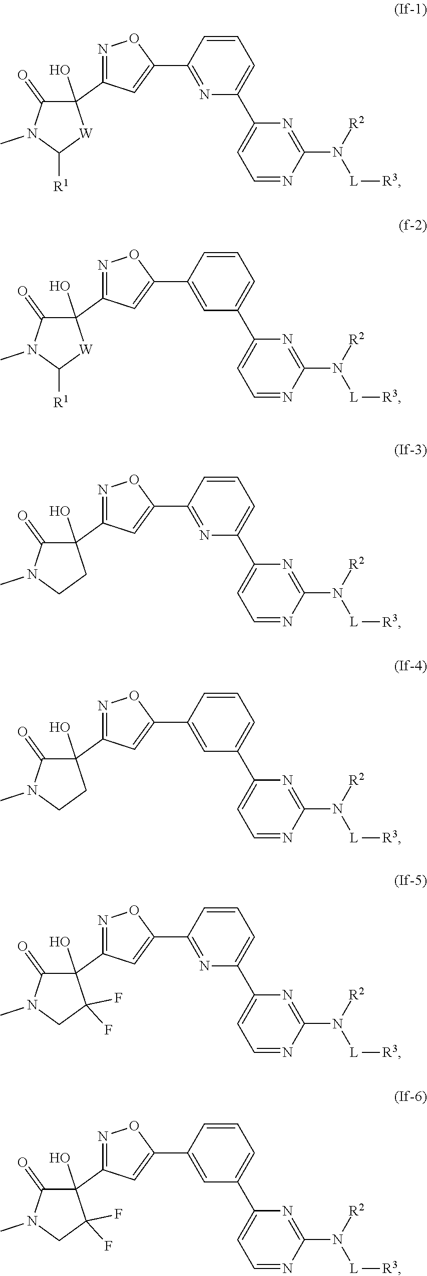

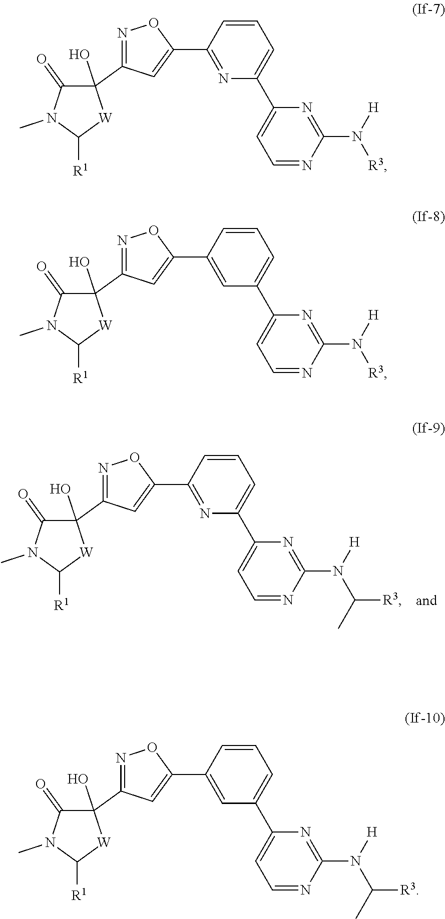

In some embodiments, disclosed herein is a compound of Formula I′ or Formula I, or a pharmaceutically acceptable salt thereof, wherein R³ is —C₍₃₋₁₀₎cycloalkyl, 3- to 10-membered heterocyclyl, a 5- to 12-membered bi- or tricyclic ring system containing one or more heteroatoms, or 5- to 10-membered heteroaryl, each of which is optionally substituted with one to five R^{3x} groups.

In some embodiments, disclosed herein is a compound of Formula I′ or Formula I, or a pharmaceutically acceptable salt thereof, wherein R³ is a 5- to 12-membered bicyclic ring system containing one or more heteroatoms or a 5- to 10-membered heteroaryl, each of which is optionally substituted with one to five R^{3x} groups.

In some embodiments, disclosed herein is a compound of Formula I′ or Formula I, or a pharmaceutically acceptable salt thereof, wherein each R^{3x} independently for each occurrence is halo, —OH, —N(R^{N1})(R^{N2}), —CN, —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, —C₍₁₋₃₎alkylC₍₃₋₁₀₎cycloalkyl, —C₍₀₋₃₎alkylC(O)N(R^{N1})(R^{N2}), 3- to 8-membered heterocyclyl, —C₍₁₋₃₎alkyl(3- to 8-membered heterocyclyl), —C₍₆₋₁₀₎aryl, or 5- to 10-membered heteroaryl, wherein the —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, —C₍₁₋₃₎alkylC₍₃₋₁₀₎cycloalkyl, —C(O)C₍₁₋₄₎alkyl, —S(O)₂C₍₁₋₄₎alkyl, —N(H)S(O)₂C₍₁₋₄₎alkyl, 3- to 8-membered heterocyclyl, —C₍₁₋₃₎alkyl(3- to 8-membered heterocyclyl), —C₍₆₋₁₀₎aryl, and 5- to 10-membered heteroaryl are optionally further substituted with one to five groups selected from halo, —OH, —N(R^{N1})(R^{N2}), —CN, —C₍₁₋₄₎alkyl, —C₍₁₋₄₎haloalkyl, —OC₍₁₋₄₎alkyl, and —OC₍₁₋₄₎haloalkyl.

In some embodiments, disclosed herein is a compound of Formula I′ or Formula I, or a pharmaceutically acceptable salt thereof, wherein each R^{3x} independently for each occurrence is halo, —OH, —N(R^{N1})(R^{N2}), —CN, —C₍₁₋₆₎alkyl, —C₍₁₋₆₎haloalkyl, —C₍₃₋₈₎cycloalkyl, —OC₍₁₋₆₎alkyl, —OC₁₋₆₎haloalkyl, —OC₍₃₋₈₎cycloalkyl, 3- to 8-membered heterocyclyl, —C₍₁₋₃₎alkyl(3- to 8-membered heterocyclyl), or 5- to 10-membered heteroaryl, wherein the —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, —C(O)C₍₁₋₄₎alkyl, —S(O)₂C₍₁₋₄₎alkyl, —N(H)S(O)₂C₍₁₋₄₎alkyl, 3- to 8-membered heterocyclyl, —C₍₁₋₃₎alkyl(3- to 8-membered heterocyclyl), and 5- to 10-membered heteroaryl are optionally further substituted with one to five groups selected from halo, —OH, —C₍₁₋₄₎alkyl, —C₍₁₋₄₎haloalkyl, —OC₍₁₋₄₎alkyl, and —OC₍₁₋₄₎ haloalkyl.

In some embodiments, disclosed herein is a compound of Formula I′ or Formula I, or a pharmaceutically acceptable salt thereof, wherein each R^{3x} independently for each occurrence is halo, —OH, —N(R^{N1})(R^{N2}), —CN, —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, 3- to 8-membered heterocyclyl, —C₍₁₋₃₎alkyl(3- to 8-membered heterocyclyl), or 5- to 10-membered heteroaryl, wherein the —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, —C(O)C₍₁₋₄₎alkyl, —S(O)₂C₍₁₋₄₎alkyl, —N(H)S(O)₂C₍₁₋₄₎alkyl, 3- to 8-membered heterocyclyl, —C₍₁₋₃₎alkyl(3- to 8-membered heterocyclyl), and 5- to 10-membered heteroaryl are optionally further substituted with one to five groups selected from halo, —OH, —C₍₁₋₄₎alkyl, —C₍₁₋₄₎haloalkyl, —OC₍₁₋₄₎alkyl, and —OC₍₁₋₄₎ haloalkyl.

In some embodiments, disclosed herein is a compound of Formula I′ or Formula I, or a pharmaceutically acceptable salt thereof, wherein R³ is a 5- to 12-membered bicyclic ring system containing one or more heteroatoms or a 5- to 10-membered heteroaryl, wherein: the 5- to 12-membered bicyclic ring system containing one or more heteroatoms is optionally substituted with one to five R^{3d}, and the 5- to 10-membered heteroaryl is optionally substituted with one to five R^{3f} groups.

In some embodiments, disclosed herein is a compound of Formula I′ or Formula I, or a pharmaceutically acceptable salt thereof, wherein R³ is —C₍₁₋₆₎alkyl, which is optionally substituted with one to five R^{3a} groups.

In some embodiments, disclosed herein is a compound of Formula I′ or Formula I, or a pharmaceutically acceptable salt thereof, wherein each R^{3a} is independently for each occurrence halo, —OH, —N(R^{N1})(R^{N2}), —CN, —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, —C₍₁₋₃₎alkylC₍₃₋₁₀₎cycloalkyl, —C₍₃₋₁₀₎cycloalkylC₍₁₋₃₎alkyl, —C(O)C₍₁₋₄₎alkyl, —S(O)₂C₍₁₋₄₎alkyl, —N(H)S(O)₂C₍₁₋₄₎alkyl, —C₍₀₋₃₎alkylC(O)N(R^{N1})(R^{N2}), 3- to 8-membered heterocyclyl, —C₍₁₋₃₎alkyl(3- to 8-membered heterocyclyl), —C₍₆₋₁₀₎aryl, or 5- to 10-membered heteroaryl, wherein the —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, —C₍₁₋₃₎alkylC₍₃₋₁₀₎cycloalkyl, —C₍₃₋₁₀₎cycloalkylC₍₁₋₃₎alkyl, —C(O)C₍₁₋₄₎alkyl, —S(O)₂C₍₁₋₄₎alkyl, —N(H)S(O)₂C₍₁₋₄₎alkyl, 3- to 8-membered heterocyclyl, —C₍₁₋₃₎alkyl(3- to 8-membered heterocyclyl), —C₍₆₋₁₀₎aryl, and 5- to 10-membered heteroaryl are optionally further substituted with one to five groups selected from halo, —OH, —N(R^{N1})(R^{N2}), —CN, —C₍₁₋₄₎alkyl, —C₍₁₋₄₎haloalkyl, —OC₍₁₋₄₎alkyl, and —OC₍₁₋₄₎ haloalkyl.

In some embodiments, disclosed herein is a compound of Formula I′ or Formula I, or a pharmaceutically acceptable salt thereof, wherein each R^{3a} is independently for each occurrence halo, —OH, —N(R^{N1})(R^{N2}), —CN, —C₍₃₋₈₎cycloalkyl, —OC₍₁₋₆₎alkyl, —OC₍₁₋₆₎haloalkyl, —OC₍₃₋₈₎cycloalkyl, —S(O)₂C₍₁₋₄₎alkyl, —N(H)S(O)₂C₍₁₋₄₎alkyl, or 3- to 8-membered heterocyclyl, wherein the —C₍₃₋₈₎cycloalkyl, —OC₍₁₋₆₎alkyl, —OC₍₁₋₆₎haloalkyl, —OC₍₃₋₈₎cycloalkyl, and 3- to 8-membered heterocyclyl are optionally further substituted with one to five groups selected from halo, —OH, —N(R^{N1})(R^{N2}), —CN, —C₍₁₋₄₎alkyl, —C₍₁₋₄₎haloalkyl, —OC₍₁₋₄₎alkyl, and —OC₍₁₋₄₎ haloalkyl.

In some embodiments, disclosed herein is a compound of Formula I′ or Formula I, or a pharmaceutically acceptable salt thereof, wherein each R^{3a} is independently for each occurrence halo, —OH, —OC₍₁₋₆₎alkyl, or —OC₍₁₋₆₎haloalkyl.

In some embodiments, disclosed herein is a compound of Formula I′ or Formula I, or a pharmaceutically acceptable salt thereof, wherein each R^{3a} is independently for each occurrence halo, —OH, —N(R^{N1})(R^{N2}), —CN, —OC₍₁₋₆₎alkyl, or —OC₍₁₋₆₎haloalkyl.

In some embodiments, disclosed herein is a compound of Formula I′ or Formula I, or a pharmaceutically acceptable salt thereof, wherein each R^{3a} is independently for each occurrence halo, —OH, —N(R^{N1})(R^{N2}), or —CN.

In some embodiments, disclosed herein is a compound of Formula I′ or Formula I, or a pharmaceutically acceptable salt thereof, wherein each R^{3a} is independently for each occurrence halo, —OH, or —OC₍₁₋₆₎alkyl.

In some embodiments, disclosed herein is a compound of Formula I′ or Formula I, or a pharmaceutically acceptable salt thereof, wherein each R^{3a} is halo. In some embodiments, disclosed herein is a compound of Formula I′ or Formula I, or a pharmaceutically acceptable salt thereof, wherein each R^{3a} is fluorine. In some embodiments, disclosed herein is a compound of Formula I′ or Formula I, or a pharmaceutically acceptable salt thereof, wherein each R^{3a} is —OH. In some embodiments, disclosed herein is a compound of Formula I′ or Formula I, or a pharmaceutically acceptable salt thereof, wherein each R^{3a} is —OC₍₁₋₆₎alkyl. In some embodiments, disclosed herein is a compound of Formula I′ or Formula I, or a pharmaceutically acceptable salt thereof, wherein each R^{3a} is —OCH₃.

In some embodiments, disclosed herein is a compound of Formula I′ or Formula I, or a pharmaceutically acceptable salt thereof, wherein R³ is:

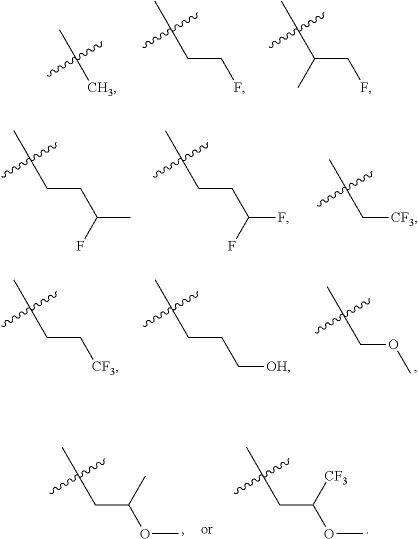

In some embodiments, disclosed herein is a compound of Formula I′ or Formula I, or a pharmaceutically acceptable salt thereof, wherein R³ is

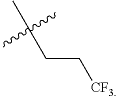 [SMILES: *CCC(F)(F)F]

In some embodiments, disclosed herein is a compound of Formula I′ or Formula I, or a pharmaceutically acceptable salt thereof, wherein R³ is —C₍₃₋₁₀₎cycloalkyl, which is optionally substituted with one to five R^{3b} groups. In some embodiments, disclosed herein is a compound of Formula I′ or Formula I, or a pharmaceutically acceptable salt thereof, wherein R³ is —C₍₃₋₁₀₎cycloalkyl, which is optionally substituted with one to three R^{3b} groups. In some embodiments, disclosed herein is a compound of Formula I′ or Formula I, or a pharmaceutically acceptable salt thereof, wherein R³ is cyclopentyl, which is optionally substituted with one to three R^{3b} groups.

In some embodiments, disclosed herein is a compound of Formula I′ or Formula I, or a pharmaceutically acceptable salt thereof, wherein each R^{3b} is independently for each occurrence halo, —OH, —N(R^{N1})(R^{N2}), —CN, —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, —C₍₁₋₃₎alkylC₍₃₋₁₀₎cycloalkyl, —C₍₃₋₁₀₎cycloalkylC₍₁₋₃₎alkyl, —C(O)C₍₁₋₄₎alkyl, —S(O)₂C₍₁₋₄₎alkyl, —N(H)S(O)₂C₍₁₋₄₎alkyl, —C₍₀₋₃₎alkylC(O)N(R^{N1})(R^{N2}), 3- to 8-membered heterocyclyl, —C₍₁₋₃₎alkyl(3- to 8-membered heterocyclyl), —C₍₆₋₁₀₎aryl, or 5- to 10-membered heteroaryl, wherein the —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, —C₍₁₋₃₎alkylC₍₃₋₁₀₎cycloalkyl, —C₍₃₋₁₀₎cycloalkylC₍₁₋₃₎alkyl, —C(O)C₍₁₋₄₎alkyl, —S(O)₂C₍₁₋₄₎alkyl, —N(H)S(O)₂C₍₁₋₄₎alkyl, 3- to 8-membered heterocyclyl, —C₍₁₋₃₎alkyl(3- to 8-membered heterocyclyl), —C₍₆₋₁₀₎aryl, and 5- to 10-membered heteroaryl are optionally further substituted with one to five groups selected from halo, —OH, —N(R^{N1})(R^{N2}), —CN, —C₍₁₋₄₎alkyl, —C₍₁₋₄₎haloalkyl, —OC₍₁₋₄₎alkyl, and —OC₍₁₋₄₎ haloalkyl.

In some embodiments, disclosed herein is a compound of Formula I′, or a pharmaceutically acceptable salt thereof, wherein each R^{3b} is independently for each occurrence halo, —OH, —N(R^{N1})(R^{N2}), —CN, —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —C₍₁₋₃₎alkylC₍₃₋₁₀₎cycloalkyl, —C(O)C₍₁₋₄₎alkyl, —S(O)₂C₍₁₋₄₎alkyl, —S(O)₂C₍₃₋₈₎cycloalkyl, —N(H)S(O)₂C₍₁₋₄₎alkyl, —C₍₀₋₃₎alkylC(O)N(R^{N1})(R^{N2}), 3- to 8-membered heterocyclyl, or 5- to 10-membered heteroaryl, wherein the —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, 3- to 8-membered heterocyclyl, and 5- to 10-membered heteroaryl are optionally further substituted with one to five groups selected from halo, —OH, —N(R^{N1})(R^{N2}), —CN, —C₍₁₋₄₎alkyl, —C₍₁₋₄₎haloalkyl, —OC₍₁₋₄₎alkyl, and —OC₍₁₋₄₎ haloalkyl.

In some embodiments, disclosed herein is a compound of Formula I′ or Formula I, or a pharmaceutically acceptable salt thereof, wherein each R^{3b} is independently for each occurrence halo, —OH, —N(R^{N1})(R^{N2}), —CN, —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —C(O)C₍₁₋₄₎alkyl, —S(O)₂C₍₁₋₄₎alkyl, —N(H)S(O)₂C₍₁₋₄₎alkyl, —C₍₀₋₃₎alkylC(O)N(R^{N1})(R^{N2}), 3- to 8-membered heterocyclyl, or 5- to 10-membered heteroaryl, wherein the —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, 3- to 8-membered heterocyclyl, and 5- to 10-membered heteroaryl are optionally further substituted with one to five groups selected from halo, —OH, —N(R^{N1})(R^{N2}), —CN, —C₍₁₋₄₎alkyl, —C₍₁₋₄₎haloalkyl, —OC₍₁₋₄₎alkyl, and —OC₍₁₋₄₎ haloalkyl.

In some embodiments, disclosed herein is a compound of Formula I′, or a pharmaceutically acceptable salt thereof, wherein each R^{3b} is independently for each occurrence halo, —OH, —N(R^{N1})(R^{N2}), —CN, —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —C₍₁₋₃₎alkylC₃₋₁₀₎cycloalkyl, —S(O)₂C₍₁₋₄₎alkyl, or —S(O)₂C₍₃₋₈₎cycloalkyl wherein the —C₍₁₋₆₎alkyl, —C₍₁₋₆₎haloalkyl, —OC₍₁₋₆₎alkyl, and —OC₍₁₋₆₎haloalkyl are optionally further substituted with one to five groups selected from halo, —OH, —N(R^{N1})(R^{N2}), —CN, and —OC₍₁₋₄₎alkyl.

In some embodiments, disclosed herein is a compound of Formula I′ or Formula I, or a pharmaceutically acceptable salt thereof, wherein each R^{3b} is independently for each occurrence halo, —OH, —N(R^{N1})(R^{N2}), —CN, —C₍₁₋₆₎alkyl, —C₍₁₋₆₎haloalkyl, —OC₍₁₋₆₎alkyl, or —OC₍₁₋₆₎haloalkyl, wherein the —C₍₁₋₆₎alkyl, —C₍₁₋₆₎haloalkyl, —OC₍₁₋₆₎alkyl, and —OC₍₁₋₆₎haloalkyl are optionally further substituted with one to five groups selected from halo, —OH, —N(R^{N1})(RN²), and —CN.

In some embodiments, disclosed herein is a compound of Formula I′ or Formula I, or a pharmaceutically acceptable salt thereof, wherein each R^{3b} is independently for each occurrence halo, —OH, —N(R^{N1})(R^{N2}), —CN, —C₍₁₋₆₎alkyl, —C₍₁₋₆₎haloalkyl, —OC₍₁₋₆₎alkyl, or —OC₍₁₋₆₎haloalkyl.

In some embodiments, disclosed herein is a compound of Formula I′ or Formula I, or a pharmaceutically acceptable salt thereof, wherein each R^{3b} is independently for each occurrence halo, —OH, —C₍₁₋₆₎haloalkyl, or —OC₍₁₋₆₎alkyl.

In some embodiments, disclosed herein is a compound of Formula I′ or Formula I, or a pharmaceutically acceptable salt thereof, wherein each R^{3b} is halo. In some embodiments, disclosed herein is a compound of Formula I′ or Formula I, or a pharmaceutically acceptable salt thereof, wherein each R^{3b} is fluorine. In some embodiments, disclosed herein is a compound of Formula I′ or Formula I, or a pharmaceutically acceptable salt thereof, wherein each R^{3b} is —OH. In some embodiments, disclosed herein is a compound of Formula I′ or Formula I, or a pharmaceutically acceptable salt thereof, wherein each R^{3b} is —C₍₁₋₆₎haloalkyl. In some embodiments, disclosed herein is a compound of Formula I′ or Formula I, or a pharmaceutically acceptable salt thereof, wherein each R^{3b} is —CF₃. In some embodiments, disclosed herein is a compound of Formula I′ or Formula I, or a pharmaceutically acceptable salt thereof, wherein each R^{3b} is —OC₍₁₋₆₎alkyl. In some embodiments, disclosed herein is a compound of Formula I′ or Formula I, or a pharmaceutically acceptable salt thereof, wherein each R^{3b} is —OCH₃.

In some embodiments, disclosed herein is a compound of Formula I′ or Formula I, or a pharmaceutically acceptable salt thereof, wherein R³ is —C₍₃₋₁₀₎cycloalkyl is optionally substituted with one —OH group.

In some embodiments, disclosed herein is a compound of Formula I′ or Formula I, or a pharmaceutically acceptable salt thereof, wherein R³ is:

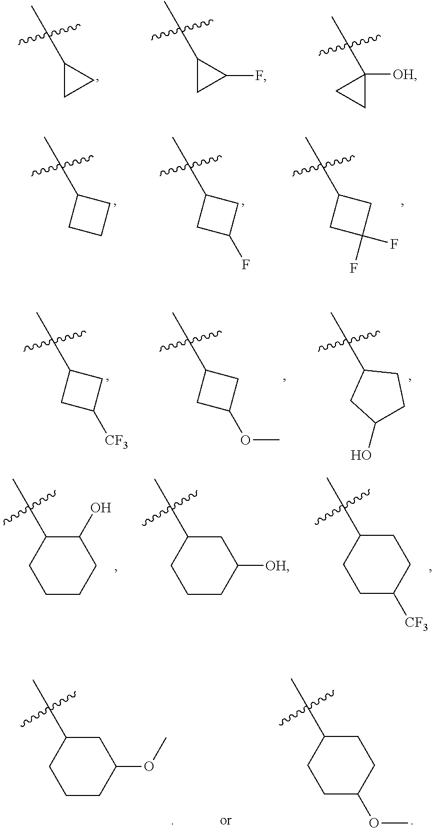

In some embodiments, disclosed herein is a compound of Formula I′ or Formula I, or a pharmaceutically acceptable salt thereof, wherein R³ is

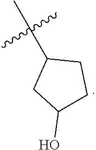 [SMILES: *C1CCC(O)C1]

In some embodiments, disclosed herein is a compound of Formula I′ or Formula I, or a pharmaceutically acceptable salt thereof, wherein R³ is 3- to 10-membered heterocyclyl, which is optionally substituted with one to five R^{3c} groups.

In some embodiments, disclosed herein is a compound of Formula I′, or a pharmaceutically acceptable salt thereof, wherein each R^{3c} is independently for each occurrence halo, —OH, —N(R^{N1})(R^{N2}), —CN, —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, —C₍₁₋₃₎alkylC₍₃₋₁₀₎cycloalkyl, —C₍₃₋₁₀₎cycloalkylC₍₁₋₃₎alkyl, —C(O)C₍₁₋₄₎alkyl, —S(O)₂C₍₁₋₄₎alkyl, —S(O)₂C₍₃₋₈₎cycloalkyl, —N(H)S(O)₂C₍₁₋₄₎alkyl, —C₍₀₋₃₎alkylC(O)N(R^{N1})(R^{N2}), 3- to 8-membered heterocyclyl, —C₍₁₋₃₎alkyl(3- to 8-membered heterocyclyl), —C₍₆₋₁₀₎aryl, or 5- to 10-membered heteroaryl, wherein the —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, —C₍₁₋₃₎alkylC₍₃₋₁₀₎cycloalkyl, —C₍₃₋₁₀₎cycloalkylC₍₁₋₃₎alkyl, —C(O)C₍₁₋₄₎alkyl, —S(O)₂C₍₁₋₄₎alkyl, —S(O)₂C₍₃₋₈₎cycloalkyl, —N(H)S(O)₂C₍₁₋₄₎alkyl, 3- to 8-membered heterocyclyl, —C₍₁₋₃₎alkyl(3- to 8-membered heterocyclyl), —C₍₆₋₁₀₎aryl, and 5- to 10-membered heteroaryl are optionally further substituted with one to five groups selected from halo, —OH, —N(R^{N1})(R^{N2}), —CN, —C₍₁₋₄₎alkyl, —C₍₁₋₄₎haloalkyl, —OC₍₁₋₄₎alkyl, —OC₍₁₋₄₎haloalkyl, and 3- to 5-membered heterocyclyl

In some embodiments, disclosed herein is a compound of Formula I′ or Formula I, or a pharmaceutically acceptable salt thereof, wherein each R^{3c} is independently for each occurrence halo, —OH, —N(R^{N1})(R^{N2}), —CN, —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, —C₍₁₋₃₎alkylC₍₃₋₁₀₎cycloalkyl, —C₍₃₋₁₀₎cycloalkylC₍₁₋₃₎alkyl, —C(O)C₍₁₋₄₎alkyl, —S(O)₂C₍₁₋₄₎alkyl, —N(H)S(O)₂C₍₁₋₄₎alkyl, —C₍₀₋₃₎alkylC(O)N(R^{N1})(R^{N2}), 3- to 8-membered heterocyclyl, —C₍₁₋₃₎alkyl(3- to 8-membered heterocyclyl), —C₍₆₋₁₀₎aryl, or 5- to 10-membered heteroaryl, wherein the —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, —C₍₁₋₃₎alkylC₍₃₋₁₀₎cycloalkyl, —C₍₃₋₁₀₎cycloalkylC₍₁₋₃₎alkyl, —C(O)C₍₁₋₄₎alkyl, —S(O)₂C₍₁₋₄₎alkyl, —N(H)S(O)₂C₍₁₋₄₎alkyl, 3- to 8-membered heterocyclyl, —C₍₁₋₃₎alkyl(3- to 8-membered heterocyclyl), —C₍₆₋₁₀₎aryl, and 5- to 10-membered heteroaryl are optionally further substituted with one to five groups selected from halo, —OH, —N(R^{N1})(R^{N2}), —CN, —C₍₁₋₄₎alkyl, —C₍₁₋₄₎haloalkyl, —OC₍₁₋₄₎alkyl, and —OC₍₁₋₄₎ haloalkyl.

In some embodiments, disclosed herein is a compound of Formula I′, or a pharmaceutically acceptable salt thereof, wherein each R^{3c} is independently for each occurrence halo, —OH, —N(R^{N1})(R^{N2}), —CN, —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —C₍₁₋₃₎alkylC₍₃₋₁₀₎cycloalkyl, —C(O)C₍₁₋₄₎alkyl, —S(O)₂C₍₁₋₄₎alkyl, —S(O)₂C₍₃₋₈₎cycloalkyl, —N(H)S(O)₂C₍₁₋₄₎alkyl, —C₍₀₋₃₎alkylC(O)N(R^{N1})(R^{N2}), 3- to 8-membered heterocyclyl, or 5- to 10-membered heteroaryl, wherein the —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, 3- to 8-membered heterocyclyl, and 5- to 10-membered heteroaryl are optionally further substituted with one to five groups selected from halo, —OH, —N(R^{N1})(R^{N2}), —CN, —C₍₁₋₄₎alkyl, —C₍₁₋₄₎haloalkyl, —OC₍₁₋₄₎alkyl, and —OC₍₁₋₄₎ haloalkyl.

In some embodiments, disclosed herein is a compound of Formula I′ or Formula I, or a pharmaceutically acceptable salt thereof, wherein each R^{3c} is independently for each occurrence halo, —OH, —N(R^{N1})(R^{N2}), —CN, —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —C(O)C₍₁₋₄₎alkyl, —S(O)₂C₍₁₋₄₎alkyl, —N(H)S(O)₂C₍₁₋₄₎alkyl, —C₍₀₋₃₎alkylC(O)N(R^{N1})(R^{N2}), 3- to 8-membered heterocyclyl, or 5- to 10-membered heteroaryl, wherein the —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, 3- to 8-membered heterocyclyl, and 5- to 10-membered heteroaryl are optionally further substituted with one to five groups selected from halo, —OH, —N(R^{N1})(R^{N2}), —CN, —C₍₁₋₄₎alkyl, —C₍₁₋₄₎haloalkyl, —OC₍₁₋₄₎alkyl, and —OC₍₁₋₄₎ haloalkyl.

In some embodiments, disclosed herein is a compound of Formula I′, or a pharmaceutically acceptable salt thereof, wherein each R^{3c} is independently for each occurrence halo, —OH, —N(R^{N1})(R^{N2}), —CN, —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —C₍₁₋₃₎alkylC₍₃₋₁₀₎cycloalkyl, —S(O)₂C₍₁₋₄₎alkyl, or —S(O)₂C₍₃₋₈₎cycloalkyl wherein the —C₍₁₋₆₎alkyl, —C₍₁₋₆₎haloalkyl, —OC₍₁₋₆₎alkyl, and —OC₍₁₋₆₎haloalkyl are optionally further substituted with one to five groups selected from halo, —OH, —N(R^{N1})(R^{N2}), —CN, and —OC₍₁₋₄₎alkyl.

In some embodiments, disclosed herein is a compound of Formula I′, or a pharmaceutically acceptable salt thereof, wherein each R^{3c} is independently for each occurrence halo, —OH, —N(R^{N1})(R^{N2}), —CN, —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —C₍₁₋₃₎alkylC₃₋₁₀₎cycloalkyl, —S(O)₂C₍₁₋₄₎alkyl, or —S(O)₂C₍₃₋₈₎cycloalkyl wherein the —C₍₁₋₆₎alkyl and —OC₍₁₋₆₎alkyl are optionally further substituted with one to five groups selected from halo and —OC₍₁₋₄₎alkyl.

In some embodiments, disclosed herein is a compound of Formula I′ or Formula I, or a pharmaceutically acceptable salt thereof, wherein each R^{3c} is independently for each occurrence halo, —OH, —N(R^{N1})(R^{N2}), —CN, —C₍₁₋₆₎alkyl, —C₍₁₋₆₎haloalkyl, —OC₍₁₋₆₎alkyl, or —OC₍₁₋₆₎haloalkyl, wherein the —C₍₁₋₆₎alkyl, —C₍₁₋₆₎haloalkyl, —OC₍₁₋₆₎alkyl, and —OC₍₁₋₆₎haloalkyl are optionally further substituted with one to five groups selected from halo, —OH, —N(R^{N1})(RN²), and —CN.

In some embodiments, disclosed herein is a compound of Formula I′, or a pharmaceutically acceptable salt thereof, wherein each R^{3c} is independently for each occurrence halo, —C₍₁₋₆₎alkyl, —C₍₁₋₆₎haloalkyl, —C₍₃₋₈₎cycloalkyl, —OC₍₁₋₆₎alkyl, —S(O)₂C₍₁₋₄₎alkyl, or —S(O)₂C₍₃₋₈₎cycloalkyl wherein the —C₍₁₋₆₎alkyl and —OC₍₁₋₆₎alkyl are optionally further substituted with one to five groups selected from halo and —OC₍₁₋₄₎alkyl.

In some embodiments, disclosed herein is a compound of Formula I′ or Formula I, or a pharmaceutically acceptable salt thereof, wherein each R^{3c} is independently for each occurrence halo. In some embodiments, disclosed herein is a compound of Formula I′ or Formula I, or a pharmaceutically acceptable salt thereof, wherein each R^{3c} is independently for each occurrence —C₍₁₋₆₎alkyl that is optionally further substituted with one to five groups selected from halo and —OC₍₁₋₄₎alkyl. In some embodiments, disclosed herein is a compound of Formula I′ or Formula I, or a pharmaceutically acceptable salt thereof, wherein each R^{3c} is independently for each occurrence —C₍₁₋₆₎haloalkyl. In some embodiments, disclosed herein is a compound of Formula I′ or Formula I, or a pharmaceutically acceptable salt thereof, wherein each R^{3c} is independently for each occurrence —C₍₃₋₈₎cycloalkyl. In some embodiments, disclosed herein is a compound of Formula I′ or Formula I, or a pharmaceutically acceptable salt thereof, wherein each R^{3c} is independently for each occurrence —OC₍₁₋₆₎alkyl that is optionally further substituted with one to five groups selected from halo and —OC₍₁₋₄₎alkyl. In some embodiments, disclosed herein is a compound of Formula I′ or Formula I, or a pharmaceutically acceptable salt thereof, wherein each R^{3c} is independently for each occurrence —S(O)₂C₍₁₋₄₎alkyl.

In some embodiments, disclosed herein is a compound of Formula I′, or a pharmaceutically acceptable salt thereof, wherein each R^{3c} is independently for each occurrence —S(O)₂C₍₃₋₈₎cycloalkyl.

In some embodiments, disclosed herein is a compound of Formula I′, or a pharmaceutically acceptable salt thereof, wherein R³ is:

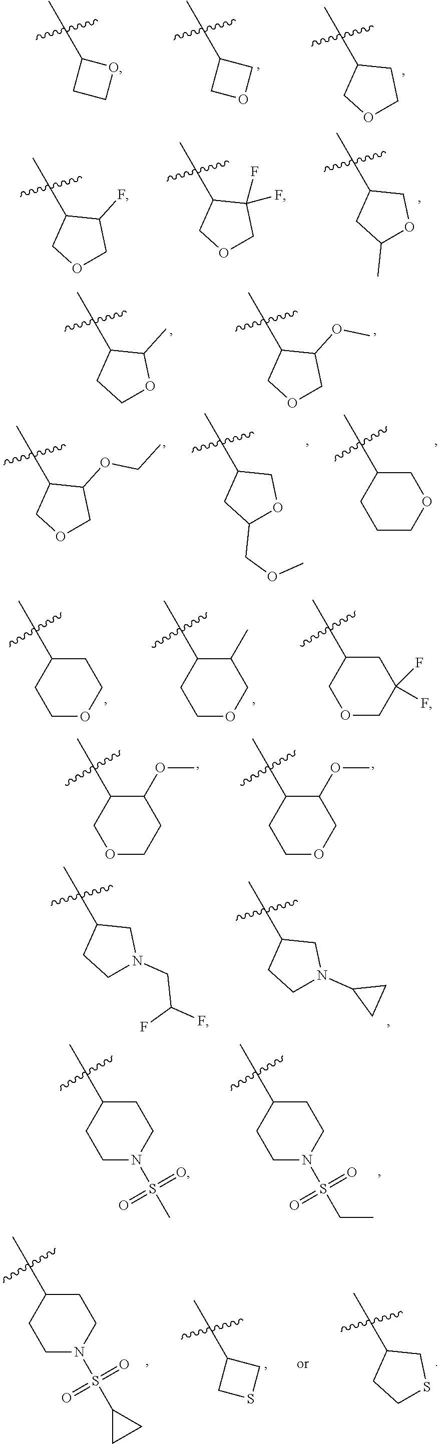

In some embodiments, disclosed herein is a compound of Formula I′ or Formula I, or a pharmaceutically acceptable salt thereof, wherein R³ is

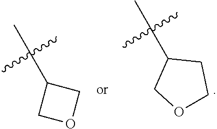 [SMILES: *C1COC1 ; *C1CCOC1]

In some embodiments, disclosed herein is a compound of Formula I′ or Formula I, or a pharmaceutically acceptable salt thereof, wherein R³ is a 5- to 12-membered bi or tricyclic ring system containing one or more heteroatoms, which is optionally substituted with one to five R^{3d} groups. In some embodiments, disclosed herein is a compound of Formula I′ or Formula I, or a pharmaceutically acceptable salt thereof, wherein R³ is a 5- to 12-membered bicyclic ring system containing one or more heteroatoms, which is optionally substituted with one to five R^{3d} groups.

In some embodiments, disclosed herein is a compound of Formula I′ or Formula I, or a pharmaceutically acceptable salt thereof, wherein R³ is a 5- to 12-membered bicyclic ring system containing one to five heteroatoms selected from O, N, and S, wherein the 5- to 12-membered bicyclic ring system is optionally substituted with one to five R^{3d} groups. In some embodiments, disclosed herein is a compound of Formula I′ or Formula I, or a pharmaceutically acceptable salt thereof, wherein R³ is a 5- to 12-membered bicyclic ring system containing one or more heteroatoms, which is optionally substituted with one to five R^{3d} groups, wherein the 5- to 12-membered bicyclic ring system is a 5,6-fused bicyclic ring system.

In some embodiments, disclosed herein is a compound of Formula I′ or Formula I, or a pharmaceutically acceptable salt thereof, wherein each R^{3d} is independently for each occurrence halo, —OH, —N(R^{N1})(R^{N2}), —CN, —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, —C₍₁₋₃₎alkylC₍₃₋₁₀₎cycloalkyl, —C₍₃₋₁₀₎cycloalkylC₍₁₋₃₎alkyl, —C(O)C₍₁₋₄₎alkyl, —S(O)₂C₍₁₋₄₎alkyl, —N(H)S(O)₂C₍₁₋₄₎alkyl, —C₍₀₋₃₎alkylC(O)N(R^{N1})(R^{N2}), 3- to 8-membered heterocyclyl, —C₍₁₋₃₎alkyl(3- to 8-membered heterocyclyl), —C₍₆₋₁₀₎aryl, or 5- to 10-membered heteroaryl, wherein the —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, —C₍₁₋₃₎alkylC₍₃₋₁₀₎cycloalkyl, —C₍₃₋₁₀₎cycloalkylC₍₁₋₃₎alkyl, —C(O)C₍₁₋₄₎alkyl, —S(O)₂C₍₁₋₄₎alkyl, —N(H)S(O)₂C₍₁₋₄₎alkyl, 3- to 8-membered heterocyclyl, —C₍₁₋₃₎alkyl(3- to 8-membered heterocyclyl), —C₍₆₋₁₀₎aryl, and 5- to 10-membered heteroaryl are optionally further substituted with one to five groups selected from halo, —OH, —N(R^{N1})(R^{N2}), —CN, —C₍₁₋₄₎alkyl, —C₍₁₋₄₎haloalkyl, —OC₍₁₋₄₎alkyl, and —OC₍₁₋₄₎ haloalkyl.

In some embodiments, disclosed herein is a compound of Formula I′ or Formula I, or a pharmaceutically acceptable salt thereof, wherein each R^{3d} is independently for each occurrence halo, —OH, —N(R^{N1})(R^{N2}), —CN, —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —C(O)C₍₁₋₄₎alkyl, —S(O)₂C₍₁₋₄₎alkyl, —N(H)S(O)₂C₍₁₋₄₎alkyl, —C₍₀₋₃₎alkylC(O)N(R^{N1})(R^{N2}), 3- to 8-membered heterocyclyl, or 5- to 10-membered heteroaryl, wherein the —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, 3- to 8-membered heterocyclyl, and 5- to 10-membered heteroaryl are optionally further substituted with one to five groups selected from halo, —OH, —N(R^{N1})(R^{N2}), —CN, —C₍₁₋₄₎alkyl, —C₍₁₋₄₎haloalkyl, —OC₍₁₋₄₎alkyl, and —OC₍₁₋₄₎ haloalkyl.

In some embodiments, disclosed herein is a compound of Formula I′, or a pharmaceutically acceptable salt thereof, wherein each R^{3d} is independently for each occurrence halo, —OH, —N(R^{N1})(R^{N2}), —CN, —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —C₍₁₋₃₎alkylC₍₃₋₁₀₎cycloalkyl, —S(O)₂C₍₁₋₄₎alkyl, or —S(O)₂C₍₃₋₈₎cycloalkyl wherein the —C₍₁₋₆₎alkyl, —C₍₁₋₆₎haloalkyl, —OC₍₁₋₆₎alkyl, and —OC₍₁₋₆₎haloalkyl are optionally further substituted with one to five groups selected from halo, —OH, —N(R^{N1})(R^{N2}), —CN, and —OC₍₁₋₄₎alkyl.

In some embodiments, disclosed herein is a compound of Formula I′ or Formula I, or a pharmaceutically acceptable salt thereof, wherein each R^{3d} is independently for each occurrence halo, —OH, —N(R^{N1})(R^{N2}), —CN, —C₍₁₋₆₎alkyl, —C₍₁₋₆₎haloalkyl, —OC₍₁₋₆₎alkyl, or —OC₍₁₋₆₎haloalkyl, wherein the —C₍₁₋₆₎alkyl, —C₍₁₋₆₎haloalkyl, —OC₍₁₋₆₎alkyl, and —OC₍₁₋₆₎haloalkyl are optionally further substituted with one to five groups selected from halo, —OH, —N(R^{N1})(RN²), and —CN.

In some embodiments, disclosed herein is a compound of Formula I′ or Formula I, or a pharmaceutically acceptable salt thereof, wherein each R^{3d} is independently for each occurrence halo, —OH, —N(R^{N1})(R^{N2}), —CN, —C₍₁₋₆₎alkyl, —C₍₁₋₆₎haloalkyl, —OC₍₁₋₆₎alkyl, —OC₍₁₋₆₎haloalkyl, or —C₍₁₋₃₎alkylC₍₃₋₁₀₎cycloalkyl.

In some embodiments, disclosed herein is a compound of Formula I′ or Formula I, or a pharmaceutically acceptable salt thereof, wherein each R^{3d} is independently for each occurrence halo, —OH, —N(R^{N1})(R^{N2}), —CN, —C₍₁₋₆₎alkyl, —C₍₁₋₆₎haloalkyl, —OC₍₁₋₆₎alkyl, or —OC₍₁₋₆₎haloalkyl.

In some embodiments, disclosed herein is a compound of Formula I′ or Formula I, or a pharmaceutically acceptable salt thereof, wherein each R^{3d} is independently for each occurrence —C₍₁₋₆₎alkyl, —C₍₁₋₆₎haloalkyl, or —C₍₁₋₃₎alkylC₍₃₋₁₀₎cycloalkyl.

In some embodiments, disclosed herein is a compound of Formula I′ or Formula I, or a pharmaceutically acceptable salt thereof, wherein each R^{3d} is —C₍₁₋₆₎alkyl. In some embodiments, disclosed herein is a compound of Formula I′ or Formula I, or a pharmaceutically acceptable salt thereof, wherein each R^{3d} is methyl. In some embodiments, disclosed herein is a compound of Formula I′ or Formula I, or a pharmaceutically acceptable salt thereof, wherein each R^{3d} is —C₍₁₋₆₎haloalkyl. In some embodiments, disclosed herein is a compound of Formula I′ or Formula I, or a pharmaceutically acceptable salt thereof, wherein each R^{3d} is —CH₂CF₃. In some embodiments, disclosed herein is a compound of Formula I′ or Formula I, or a pharmaceutically acceptable salt thereof, wherein each R^{3d} is —C₍₁₋₃₎alkylC₍₃₋₁₀₎cycloalkyl. In some embodiments, disclosed herein is a compound of Formula I′ or Formula I, or a pharmaceutically acceptable salt thereof, wherein each R^{3d} is —CH₂-cyclopropyl.

In some embodiments, disclosed herein is a compound of Formula I′ or Formula I, or a pharmaceutically acceptable salt thereof, wherein R³ is:

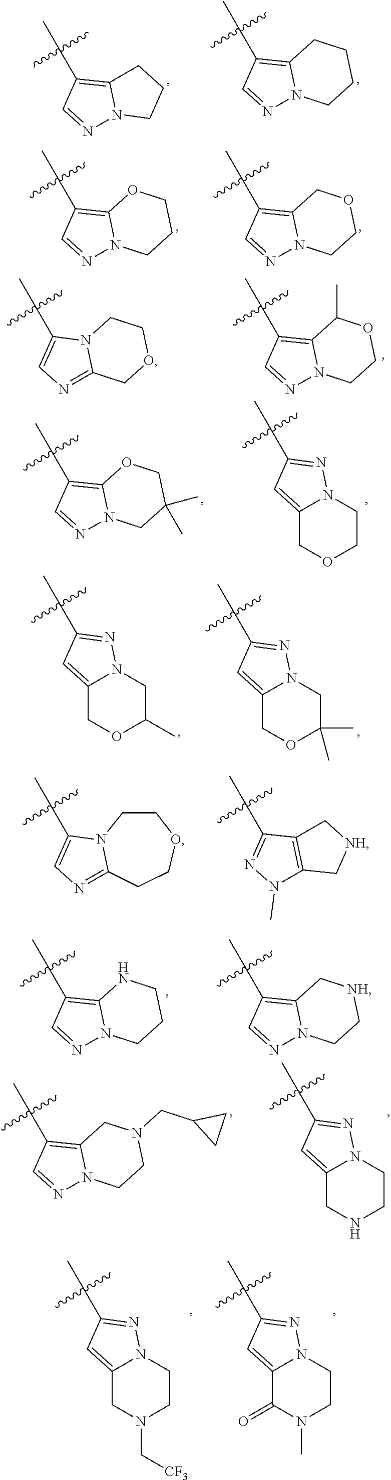

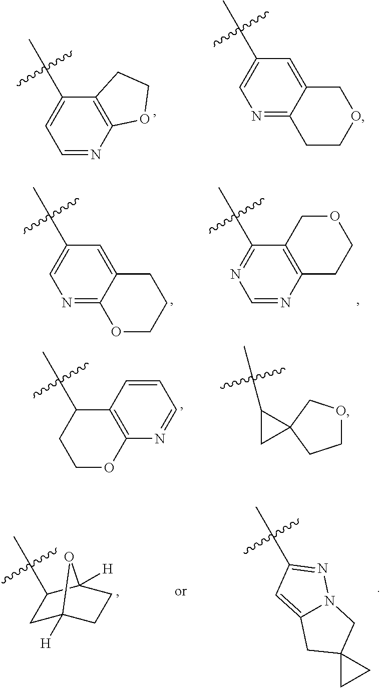

In some embodiments, disclosed herein is a compound of Formula I′ or Formula I, or a pharmaceutically acceptable salt thereof, wherein R³ is:

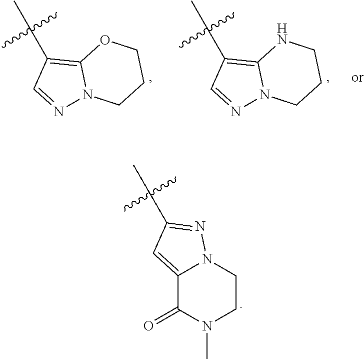 [SMILES: *c1cnn2c1OCCC2 ; *c1cnn2c1NCCC2 ; *c1cc2n(n1)CCN(C)C2=O]

In some embodiments, disclosed herein is a compound of Formula I′ or Formula I, or a pharmaceutically acceptable salt thereof, wherein R³ is —C₍₆₋₁₀₎aryl, which is optionally substituted with one to five R^{3e} groups. In some embodiments, disclosed herein is a compound of Formula I′ or Formula I, or a pharmaceutically acceptable salt thereof, wherein R³ is phenyl.

In some embodiments, disclosed herein is a compound of Formula I′ or Formula I, or a pharmaceutically acceptable salt thereof, wherein each R^{3e} is independently for each occurrence halo, —OH, —N(R^{N1})(R^{N2}), —CN, —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, —C₍₁₋₃₎alkylC₍₃₋₁₀₎cycloalkyl, —C₍₃₋₁₀₎cycloalkylC₍₁₋₃₎alkyl, —C(O)C₍₁₋₄₎alkyl, —S(O)₂C₍₁₋₄₎alkyl, —N(H)S(O)₂C₍₁₋₄₎alkyl, —C₍₀₋₃₎alkylC(O)N(R^{N1})(R^{N2}), 3- to 8-membered heterocyclyl, —C₍₁₋₃₎alkyl(3- to 8-membered heterocyclyl), —C₍₆₋₁₀₎aryl, or 5- to 10-membered heteroaryl, wherein the —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, —C₍₁₋₃₎alkylC₍₃₋₁₀₎cycloalkyl, —C₍₃₋₁₀₎cycloalkylC₍₁₋₃₎alkyl, —C(O)C₍₁₋₄₎alkyl, —S(O)₂C₍₁₋₄₎alkyl, —N(H)S(O)₂C₍₁₋₄₎alkyl, 3- to 8-membered heterocyclyl, —C₍₁₋₃₎alkyl(3- to 8-membered heterocyclyl), —C₍₆₋₁₀₎aryl, and 5- to 10-membered heteroaryl are optionally further substituted with one to five groups selected from halo, —OH, —N(R^{N1})(R^{N2}), —CN, —C₍₁₋₄₎alkyl, —C₍₁₋₄₎haloalkyl, —OC₍₁₋₄₎alkyl, and —OC₍₁₋₄₎ haloalkyl.

In some embodiments, disclosed herein is a compound of Formula I′ or Formula I, or a pharmaceutically acceptable salt thereof, wherein each R^{3e} is independently for each occurrence halo, —OH, —N(R^{N1})(R^{N2}), —CN, —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, —C₍₁₋₃₎alkylC₍₃₋₁₀₎cycloalkyl, —C(O)C₍₁₋₄₎alkyl, —S(O)₂C₍₁₋₄₎alkyl, —N(H)S(O)₂C₍₁₋₄₎alkyl, —C₍₀₋₃₎alkylC(O)N(R^{N1})(R^{N2}), 3- to 8-membered heterocyclyl, —C₍₁₋₃₎alkyl(3- to 8-membered heterocyclyl), —C₍₆₋₁₀₎aryl, or 5- to 10-membered heteroaryl, wherein the —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, —C₍₁₋₃₎alkylC₍₃₋₁₀₎cycloalkyl, 3- to 8-membered heterocyclyl, —C₍₁₋₃₎alkyl(3- to 8-membered heterocyclyl), —C₍₆₋₁₀₎aryl, and 5- to 10-membered heteroaryl are optionally further substituted with one to five groups selected from halo, —OH, —N(R^{N1})(R^{N2}), —CN, —C₍₁₋₄₎alkyl, —C₍₁₋₄₎haloalkyl, —OC₍₁₋₄₎alkyl, and —OC₍₁₋₄₎haloalkyl.

In some embodiments, disclosed herein is a compound of Formula I′ or Formula I, or a pharmaceutically acceptable salt thereof, wherein each R^{3e} is independently for each occurrence halo, —OH, —N(R^{N1})(R^{N2}), —CN, —C₍₁₋₆₎alkyl, —C₍₁₋₆₎haloalkyl, —OC₍₁₋₆₎alkyl, or —OC₍₁₋₆₎haloalkyl.

In some embodiments, disclosed herein is a compound of Formula I′ or Formula I, or a pharmaceutically acceptable salt thereof, wherein R³ is 5- to 10-membered heteroaryl, which is optionally substituted with one to five R^{3f} groups. In some embodiments, disclosed herein is a compound of Formula I′ or Formula I, or a pharmaceutically acceptable salt thereof, wherein R³ is a 5- to 6-membered monocyclic heteroaryl or a 9-membered bicyclic heteroaryl, each of which is optionally substituted with one to three R^{3f} groups.

In some embodiments, disclosed herein is a compound of Formula I′ or Formula I, or a pharmaceutically acceptable salt thereof, wherein R³ is pyrazolyl, imidazolyl, triazolyl, tetrazolyl, oxazolyl, isoxazolyl, oxadiazolyl, thiazolyl, isothiazolyl, pyridinyl, pyridazinyl, pyrimidinyl, pyrazinyl, triazinyl, or a 9-membered fused nitrogen-containing bicyclic heteroaryl, each of which is optionally substituted with one to three R^{3f} groups. In some embodiments, disclosed herein is a compound of Formula I′ or Formula I, or a pharmaceutically acceptable salt thereof, wherein R³ is pyrazolyl, imidazolyl, triazolyl, oxazolyl, isoxazolyl, pyridinyl, pyrazolopyridinyl, or pyrazolopyrimidinyl, each of which is optionally substituted with one to three R^{3f} groups. In some embodiments, disclosed herein is a compound of Formula I′ or Formula I, or a pharmaceutically acceptable salt thereof, wherein R³ is pyrazolyl, triazolyl, isoxazolyl, pyridinyl, pyrazolopyridinyl, or pyrazolopyrimidinyl, each of which is optionally substituted with one to three R^{3f} groups.

In some embodiments, disclosed herein is a compound of Formula I′ or Formula I, or a pharmaceutically acceptable salt thereof, wherein R³ is pyrazolyl, isoxazolyl, or pyrazolopyrimidinyl, each of which is optionally substituted with one to three R^{3f} groups. In some embodiments, disclosed herein is a compound of Formula I′ or Formula I, or a pharmaceutically acceptable salt thereof, wherein R³ is pyrazolyl, which is optionally substituted with one to three R^{3f} groups. In some embodiments, disclosed herein is a compound of Formula I′ or Formula I, or a pharmaceutically acceptable salt thereof, wherein R³ is isoxazolyl, which is optionally substituted with one to three R^{3f} groups. In some embodiments, disclosed herein is a compound of Formula I′ or Formula I, or a pharmaceutically acceptable salt thereof, wherein R³ is pyridinyl, which is optionally substituted with one to three R^{3f} groups. In some embodiments, disclosed herein is a compound of Formula I′ or Formula I, or a pharmaceutically acceptable salt thereof, wherein R³ is pyrazolopyrimidinyl, which is optionally substituted with one to three R^{3f} groups.

In some embodiments, disclosed herein is a compound of Formula I′ or Formula I, or a pharmaceutically acceptable salt thereof, wherein R³ is:

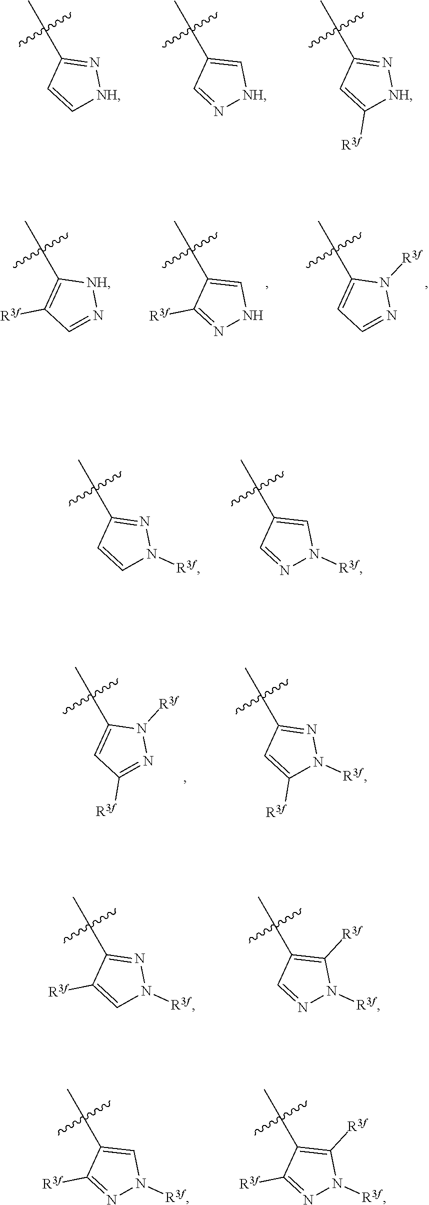

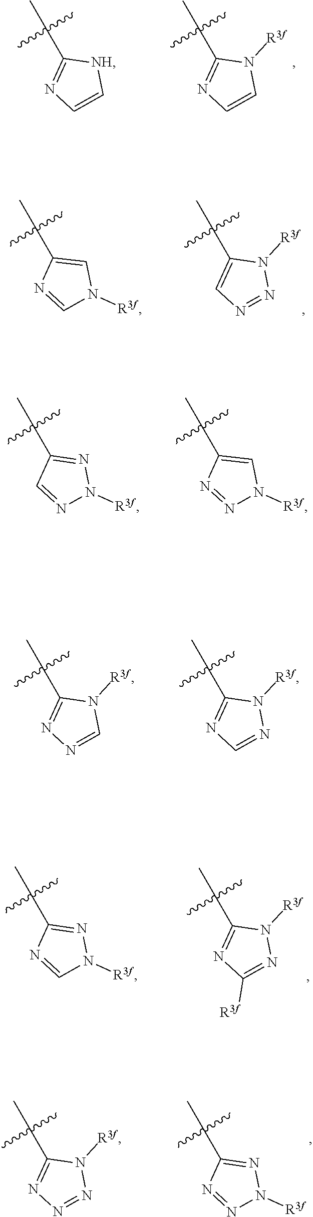

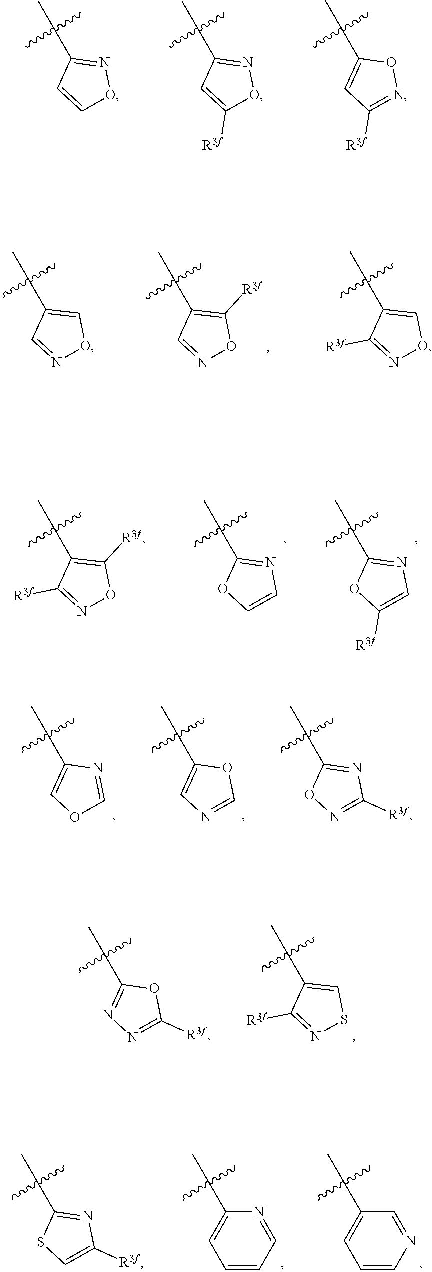

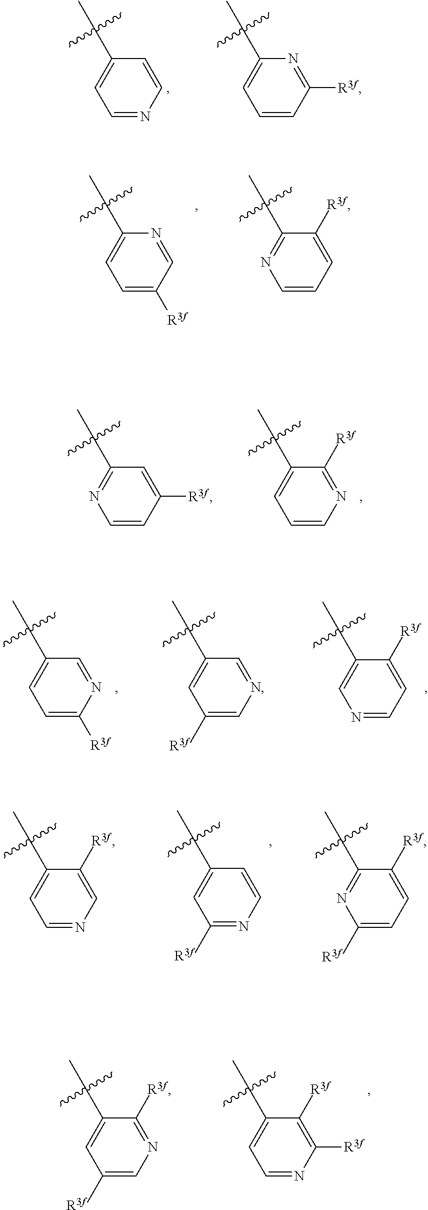

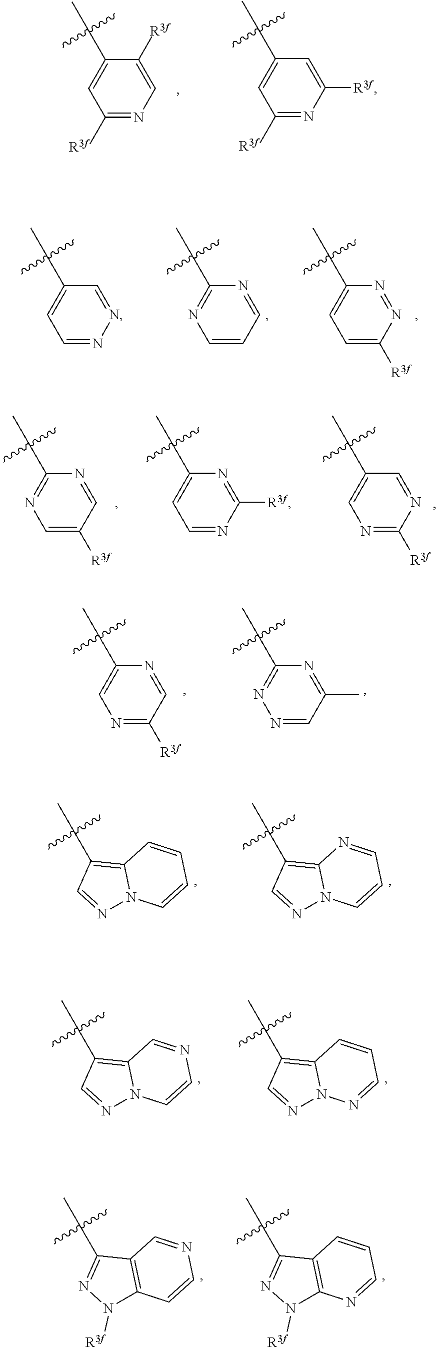

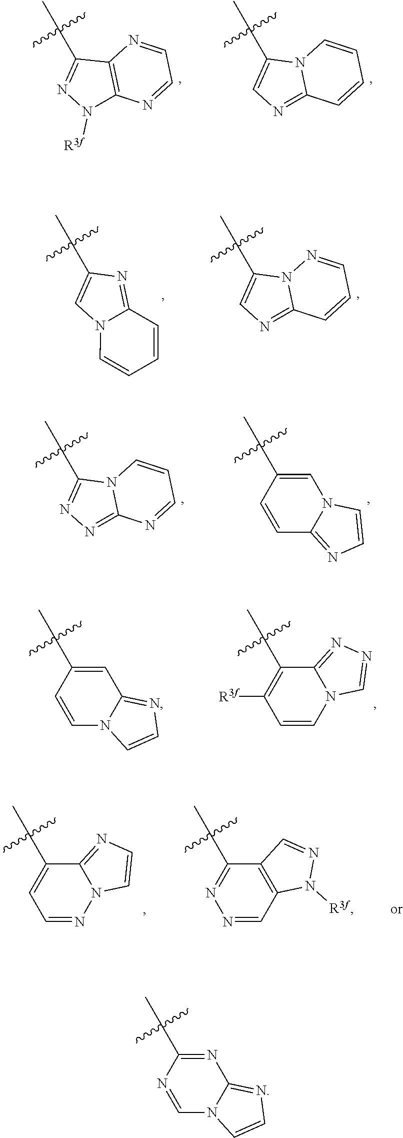

In some embodiments, disclosed herein is a compound of Formula I′ or Formula I, or a pharmaceutically acceptable salt thereof, wherein R³ is:

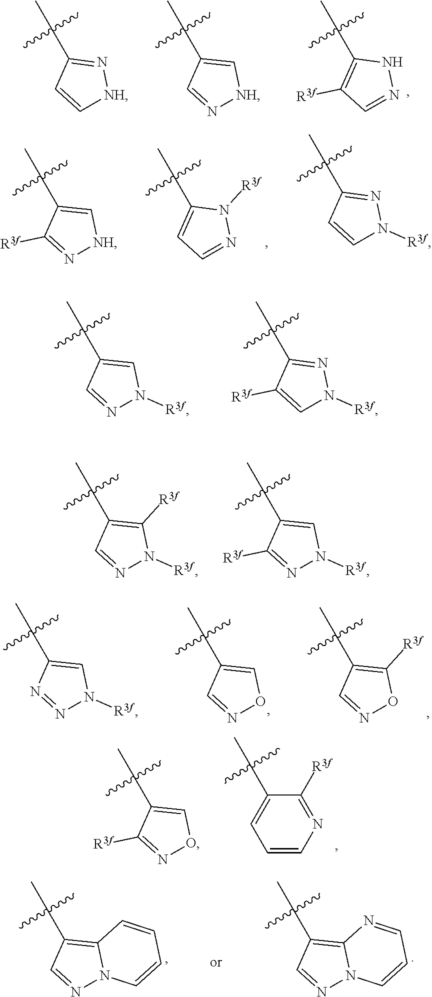

In some embodiments, disclosed herein is a compound of Formula I′ or Formula I, or a pharmaceutically acceptable salt thereof, wherein R³ is:

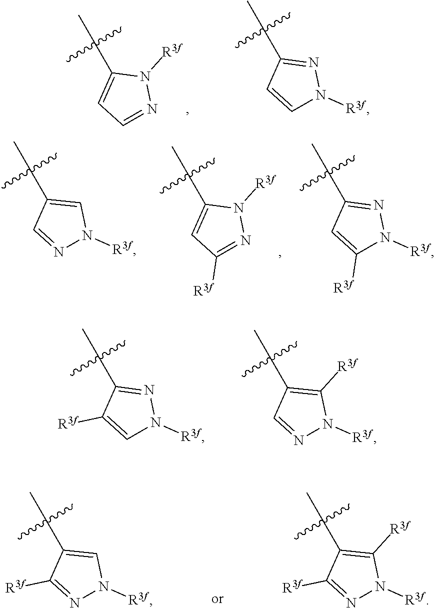

In some embodiments, disclosed herein is a compound of Formula I′ or Formula I, or a pharmaceutically acceptable salt thereof, wherein R³ is:

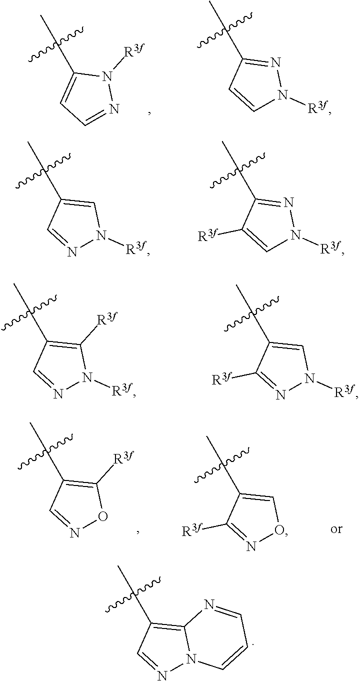

In some embodiments, disclosed herein is a compound of Formula I′ or Formula I, or a pharmaceutically acceptable salt thereof, wherein R³ is:

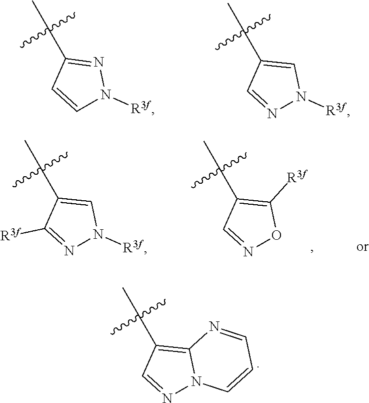

In some embodiments, disclosed herein is a compound of Formula I′ or Formula I, or a pharmaceutically acceptable salt thereof, wherein R³ is:

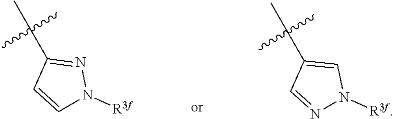

In some embodiments, disclosed herein is a compound of Formula I′ or Formula I, or a pharmaceutically acceptable salt thereof, wherein each R^{3f} is independently for each occurrence halo, —OH, —N(R^{N1})(R^{N2}), —CN, —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, —C₍₁₋₃₎alkylC₍₃₋₁₀₎cycloalkyl, —C₍₃₋₁₀₎cycloalkylC₍₁₋₃₎alkyl, —C(O)C₍₁₋₄₎alkyl, —S(O)₂C₍₁₋₄₎alkyl, —N(H)S(O)₂C₍₁₋₄₎alkyl, —C₍₀₋₃₎alkylC(O)N(R^{N1})(R^{N2}), 3- to 8-membered heterocyclyl, —C₍₁₋₃₎alkyl(3- to 8-membered heterocyclyl), —C₍₆₋₁₀₎aryl, or 5- to 10-membered heteroaryl, wherein the —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, —C₍₁₋₃₎alkylC₍₃₋₁₀₎cycloalkyl, —C₍₃₋₁₀₎cycloalkylC₍₁₋₃₎alkyl, —C(O)C₍₁₋₄₎alkyl, —S(O)₂C₍₁₋₄₎alkyl, —N(H)S(O)₂C₍₁₋₄₎alkyl, 3- to 8-membered heterocyclyl, —C₍₁₋₃₎alkyl(3- to 8-membered heterocyclyl), —C₍₆₋₁₀₎aryl, and 5- to 10-membered heteroaryl are optionally further substituted with one to five groups selected from halo, —OH, —N(R^{N1})(R^{N2}), —CN, —C₍₁₋₄₎alkyl, —C₍₁₋₄₎haloalkyl, —OC₍₁₋₄₎alkyl, and —OC₍₁₋₄₎ haloalkyl.

In some embodiments, disclosed herein is a compound of Formula I′, or a pharmaceutically acceptable salt thereof, wherein each R^{3f} is independently for each occurrence halo, —OH, —N(R^{N1})(R^{N2}), —CN, —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, —C₍₁₋₃₎alkylC₍₃₋₁₀₎cycloalkyl, —C(O)C₍₁₋₄₎alkyl, —S(O)₂C₍₁₋₄₎alkyl, —N(H)S(O)₂C₍₁₋₄₎alkyl, —C₍₀₋₃₎alkylC(O)N(R^{N1})(R^{N2}), 3- to 8-membered heterocyclyl, —C₍₁₋₃₎alkyl(3- to 8-membered heterocyclyl), —C₍₆₋₁₀₎aryl, or 5- to 10-membered heteroaryl, wherein the —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, —C₍₁₋₃₎alkylC₍₃₋₁₀₎cycloalkyl, 3- to 8-membered heterocyclyl, —C₍₁₋₃₎alkyl(3- to 8-membered heterocyclyl), —C₍₆₋₁₀₎aryl, and 5- to 10-membered heteroaryl are optionally further substituted with one to five groups selected from halo, —OH, —N(R^{N1})(R^{N2}), —CN, —C₍₁₋₄₎alkyl, —C₍₁₋₄₎haloalkyl, —OC₍₁₋₄₎alkyl, —OC₍₁₋₄₎haloalkyl, and 3- to 5-membered heterocyclyl.

In some embodiments, disclosed herein is a compound of Formula I′ or Formula I, or a pharmaceutically acceptable salt thereof, wherein each R^{3f} is independently for each occurrence halo, —OH, —N(R^{N1})(R^{N2}), —CN, —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, —C₍₁₋₃₎alkylC₍₃₋₁₀₎cycloalkyl, —C(O)C₍₁₋₄₎alkyl, —S(O)₂C₍₁₋₄₎alkyl, —N(H)S(O)₂C₍₁₋₄₎alkyl, —C₍₀₋₃₎alkylC(O)N(R^{N1})(R^{N2}), 3- to 8-membered heterocyclyl, —C₍₁₋₃₎alkyl(3- to 8-membered heterocyclyl), —C₍₆₋₁₀₎aryl, or 5- to 10-membered heteroaryl, wherein the —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, —C₍₁₋₃₎alkylC₍₃₋₁₀₎cycloalkyl, 3- to 8-membered heterocyclyl, —C₍₁₋₃₎alkyl(3- to 8-membered heterocyclyl), —C₍₆₋₁₀₎aryl, and 5- to 10-membered heteroaryl are optionally further substituted with one to five groups selected from halo, —OH, —N(R^{N1})(R^{N2}), —CN, —C₍₁₋₄₎alkyl, —C₍₁₋₄₎haloalkyl, —OC₍₁₋₄₎alkyl, and —OC₍₁₋₄₎haloalkyl.

In some embodiments, disclosed herein is a compound of Formula I′, or a pharmaceutically acceptable salt thereof, wherein each R^{3f} is independently for each occurrence halo, —OH, —N(R^{N1})(R^{N2}), —CN, —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, —C₍₁₋₃₎alkylC₍₃₋₁₀₎cycloalkyl, —C₍₀₋₃₎alkylC(O)N(R^{N1})(R^{N2}), 3- to 8-membered heterocyclyl, —C₍₁₋₃₎alkyl(3- to 8-membered heterocyclyl), —C₍₆₋₁₀₎aryl, or 5- to 10-membered heteroaryl, wherein the —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, —C₍₁₋₃₎alkylC₍₃₋₁₀₎cycloalkyl, 3- to 8-membered heterocyclyl, —C₍₁₋₃₎alkyl(3- to 8-membered heterocyclyl), —C₍₆₋₁₀₎aryl, and 5- to 10-membered heteroaryl are optionally further substituted with one to five groups selected from halo, —OH, —N(R^{N1})(R^{N2}), —CN, —C₍₁₋₄₎alkyl, —C₍₁₋₄₎haloalkyl, —OC₍₁₋₄₎alkyl, —OC₍₁₋₄₎ haloalkyl, and 3- to 5-membered heterocyclyl In some embodiments, disclosed herein is a compound of Formula I′ or Formula I, or a pharmaceutically acceptable salt thereof, wherein each R^{3f} is independently for each occurrence halo, —OH, —N(R^{N1})(R^{N2}), —CN, —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, —C₍₁₋₃₎alkylC₍₃₋₁₀₎cycloalkyl, —C₍₀₋₃₎alkylC(O)N(R^{N1})(R^{N2}), 3- to 8-membered heterocyclyl, —C₍₁₋₃₎alkyl(3- to 8-membered heterocyclyl), or —C₍₆₋₁₀₎aryl, wherein the —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, —C₍₁₋₃₎alkylC₍₃₋₁₀₎cycloalkyl, 3- to 8-membered heterocyclyl, —C₍₁₋₃₎alkyl(3- to 8-membered heterocyclyl), and —C₍₆₋₁₀₎aryl are optionally further substituted with one to five groups selected from halo, —OH, —N(R^{N1})(R^{N2}), —CN, —C₍₁₋₄₎alkyl, —C₍₁₋₄₎haloalkyl, —OC₍₁₋₄₎alkyl, and —OC₍₁₋₄₎ haloalkyl.

In some embodiments, disclosed herein is a compound of Formula I′ or Formula I, or a pharmaceutically acceptable salt thereof, wherein each R^{3f} is independently for each occurrence halo, —OH, —N(R^{N1})(R^{N2}), —CN, —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, —C₍₀₋₃₎)alkylC(O)N(R^{N1})(R^{N2}), 3- to 8-membered heterocyclyl, —C₍₁₋₃₎alkyl(3- to 8-membered heterocyclyl), or 5- to 10-membered heteroaryl, wherein the —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, 3- to 8-membered heterocyclyl, —C₍₁₋₃₎alkyl(3- to 8-membered heterocyclyl), and 5- to 10-membered heteroaryl are optionally further substituted with one to five groups selected from halo, —OH, —N(R^{N1})(R^{N2}), —C₍₁₋₄₎alkyl, —C₍₁₋₄₎haloalkyl, —OC₍₁₋₄₎alkyl, —OC₍₁₋₄₎ haloalkyl, and 3- to 5-membered heterocyclyl.

In some embodiments, disclosed herein is a compound of Formula I′ or Formula I, or a pharmaceutically acceptable salt thereof, wherein each R^{3f} is independently for each occurrence halo, —OH, —N(R^{N1})(R^{N2}), —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, 3- to 8-membered heterocyclyl, or —C₍₁₋₃₎alkyl(3- to 8-membered heterocyclyl), wherein the —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, 3- to 8-membered heterocyclyl, and —C₍₁₋₃₎alkyl(3- to 8-membered heterocyclyl) are optionally further substituted with one to five groups selected from halo, —OH, —C₍₁₋₄₎alkyl, —C₍₁₋₄₎haloalkyl, —OC₍₁₋₄₎alkyl, and —OC₍₁₋₄₎ haloalkyl.

In some embodiments, disclosed herein is a compound of Formula I′ or Formula I, or a pharmaceutically acceptable salt thereof, wherein each R^{3f} is independently for each occurrence —N(R^{N1})(R^{N2}), —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, 3- to 8-membered heterocyclyl, or —C₍₁₋₃₎alkyl(3- to 8-membered heterocyclyl), wherein the —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, 3- to 8-membered heterocyclyl, and —C₍₁₋₃₎alkyl(3- to 8-membered heterocyclyl) are optionally further substituted with one to five groups selected from halo, —OH, —C₍₁₋₄₎alkyl, —C₍₁₋₄₎haloalkyl, —OC₍₁₋₄₎alkyl, and —OC₍₁₋₄₎haloalkyl.

In some embodiments, disclosed herein is a compound of Formula I′ or Formula I, or a pharmaceutically acceptable salt thereof, wherein each R^{3f} is independently for each occurrence halo, —C₍₁₋₆₎alkyl, —OC₍₃₋₈₎cycloalkyl, —OC₍₁₋₆₎alkyl, or —OC₍₃₋₈₎cycloalkyl, wherein the —C₍₁₋₆₎alkyl, —OC₍₃₋₈₎cycloalkyl, —OC₍₁₋₆₎alkyl, and —OC₍₃₋₈₎cycloalkyl are optionally further substituted with one to five groups selected from halo and —OH.

In some embodiments, disclosed herein is a compound of Formula I′ or Formula I, or a pharmaceutically acceptable salt thereof, wherein each R^{3f} is independently for each occurrence —C₍₁₋₆₎alkyl, —C₍₁₋₆₎haloalkyl, —OC₍₃₋₈₎cycloalkyl, —OC₍₁₋₆₎alkyl, —OC₍₁₋₆₎haloalkyl, or —OC₍₃₋₈₎cycloalkyl, each of which is optionally further substituted with one to three —OH groups.

In some embodiments, disclosed herein is a compound of Formula I′ or Formula I, or a pharmaceutically acceptable salt thereof, wherein each R^{3f} is independently for each occurrence —C₍₁₋₆₎alkyl, —OC₍₃₋₈₎cycloalkyl, —OC₍₁₋₆₎alkyl, or —OC₍₃₋₈₎cycloalkyl, each of which is optionally further substituted with one to five groups selected from fluorine and —OH.

In some embodiments, disclosed herein is a compound of Formula I′ or Formula I, or a pharmaceutically acceptable salt thereof, wherein each R^{3f} is independently for each occurrence —C₍₁₋₆₎alkyl, which is optionally further substituted with one to five groups selected from fluorine and —OH. In some embodiments, disclosed herein is a compound of Formula I′ or Formula I, or a pharmaceutically acceptable salt thereof, wherein each R^{3f} is independently for each occurrence —C₍₁₋₆₎alkyl. In some embodiments, disclosed herein is a compound of Formula I′ or Formula I, or a pharmaceutically acceptable salt thereof, wherein each R^{3f} is methyl.

In some embodiments, disclosed herein is a compound of Formula I′ or Formula I, or a pharmaceutically acceptable salt thereof, wherein each R^{3f} is independently for each occurrence —OC₍₃₋₈₎cycloalkyl, which is optionally further substituted with one to five groups selected from fluorine and —OH. In some embodiments, disclosed herein is a compound of Formula I′ or Formula I, or a pharmaceutically acceptable salt thereof, wherein each R³ is independently for each occurrence —OC₍₃₋₈₎cycloalkyl.

In some embodiments, disclosed herein is a compound of Formula I′ or Formula I, or a pharmaceutically acceptable salt thereof, wherein each R^{3f} is independently for each occurrence —OC₍₁₋₆₎alkyl, which is optionally further substituted with one to five groups selected from fluorine and —OH. In some embodiments, disclosed herein is a compound of Formula I′ or Formula I, or a pharmaceutically acceptable salt thereof, wherein each R^{3f} is independently for each occurrence —OC₍₁₋₆₎alkyl.

In some embodiments, disclosed herein is a compound of Formula I′ or Formula I, or a pharmaceutically acceptable salt thereof, wherein each R^{3f} is independently for each occurrence —OC₍₃₋₈₎cycloalkyl, which is optionally further substituted with one to five groups selected from fluorine and —OH. In some embodiments, disclosed herein is a compound of Formula I′ or Formula I, or a pharmaceutically acceptable salt thereof, wherein each R³ is independently for each occurrence —OC₍₃₋₈₎cycloalkyl.

In some embodiments, disclosed herein is a compound of Formula I′, or a pharmaceutically acceptable salt thereof, wherein each R^{3f} is independently for each occurrence:

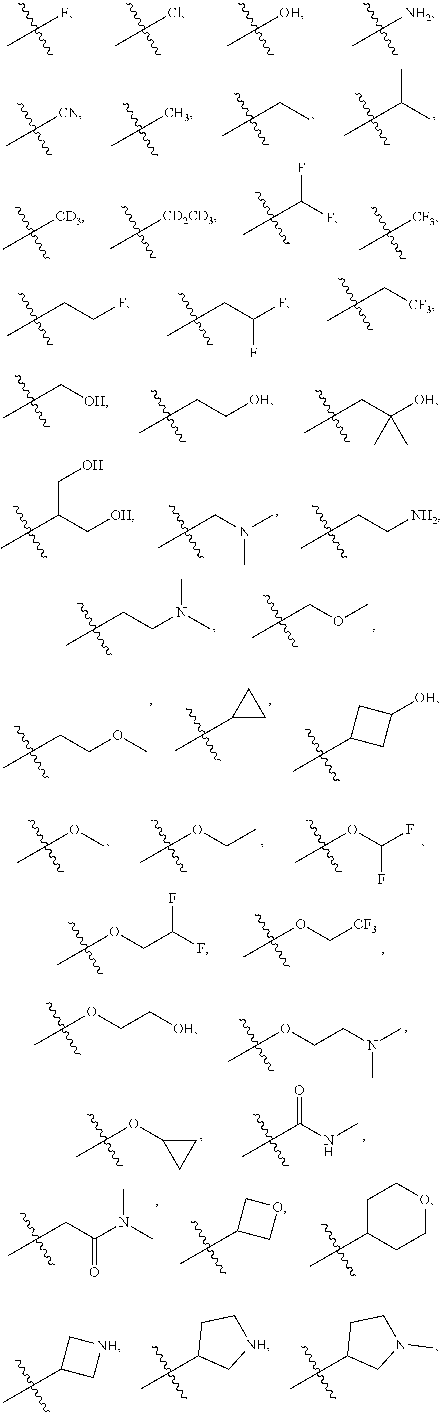

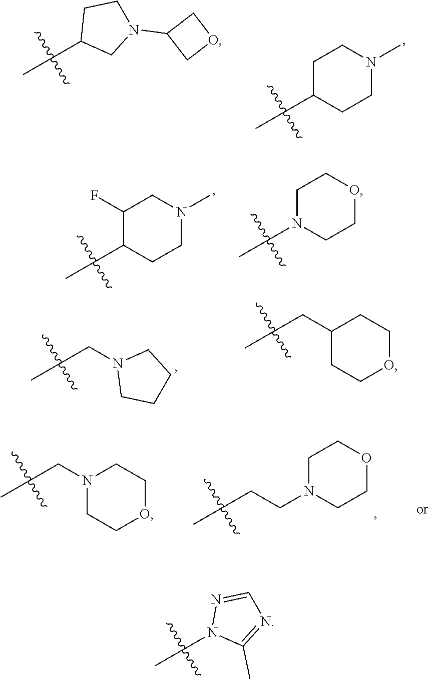

In some embodiments, disclosed herein is a compound of Formula I′ or Formula I, or a pharmaceutically acceptable salt thereof, wherein each R^{3f} is independently for each occurrence:

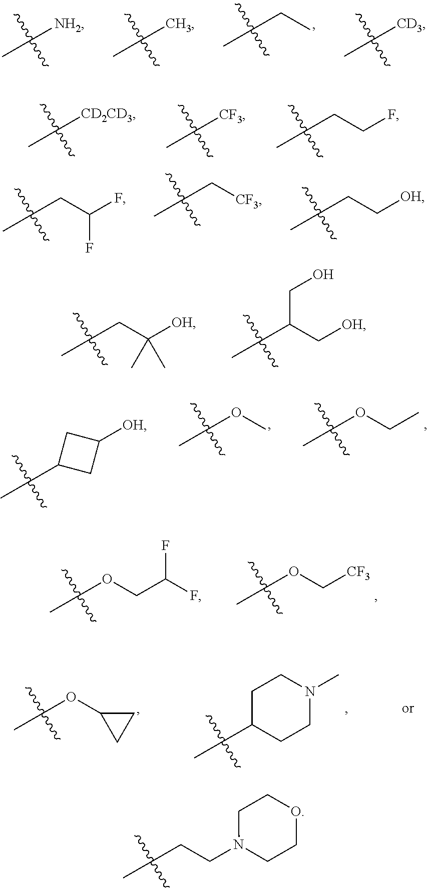

In some embodiments, disclosed herein is a compound of Formula I′ or Formula I, or a pharmaceutically acceptable salt thereof, wherein each R^{3f} is independently for each occurrence:

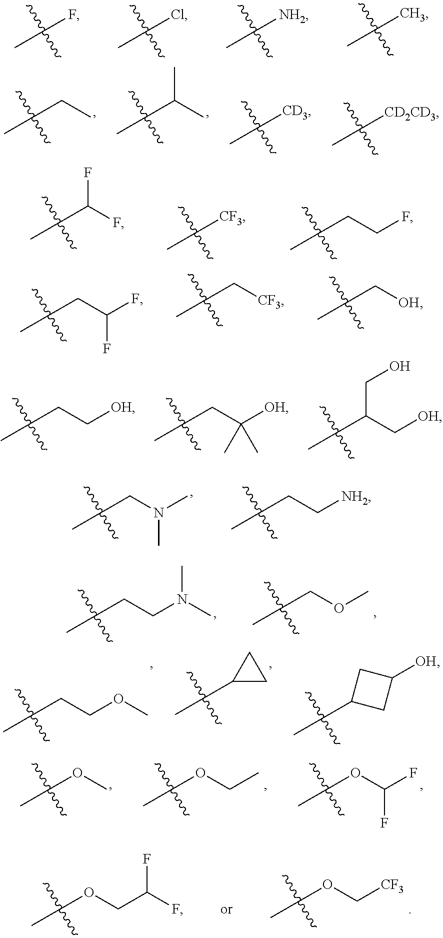

In some embodiments, disclosed herein is a compound of Formula I′ or Formula I, or a pharmaceutically acceptable salt thereof, wherein each R^{3f} is independently for each occurrence:

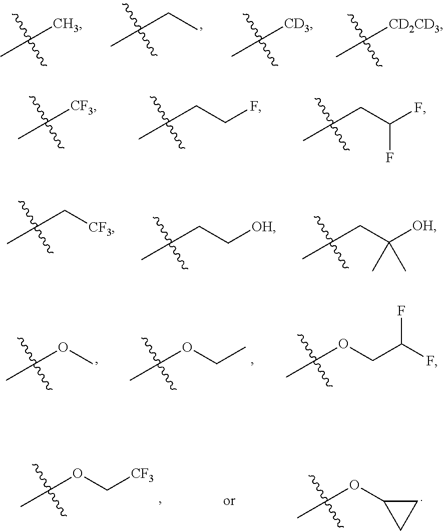

In some embodiments, disclosed herein is a compound of Formula I′ or Formula I, or a pharmaceutically acceptable salt thereof, wherein each R^{3f} is independently for each occurrence:

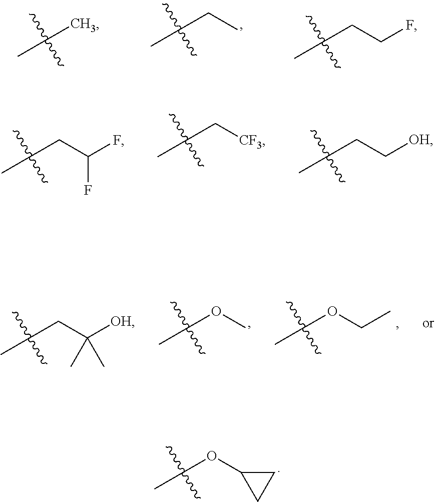

In some embodiments, disclosed herein is a compound of Formula I′ or Formula I or a pharmaceutically acceptable salt thereof, wherein R³ is:

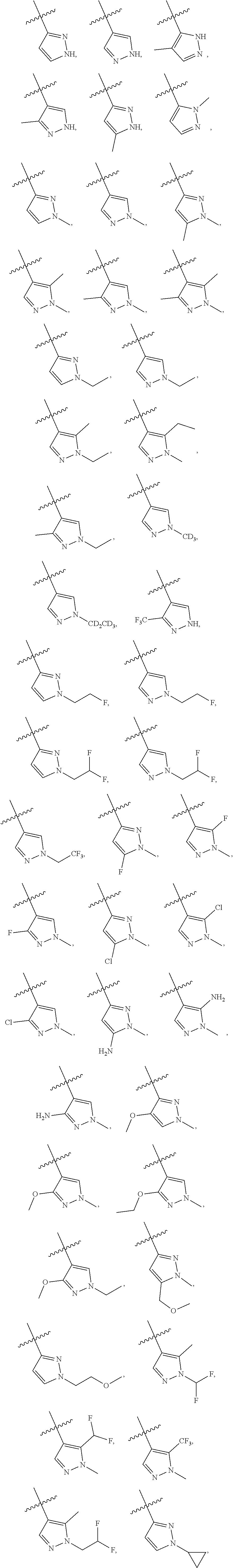

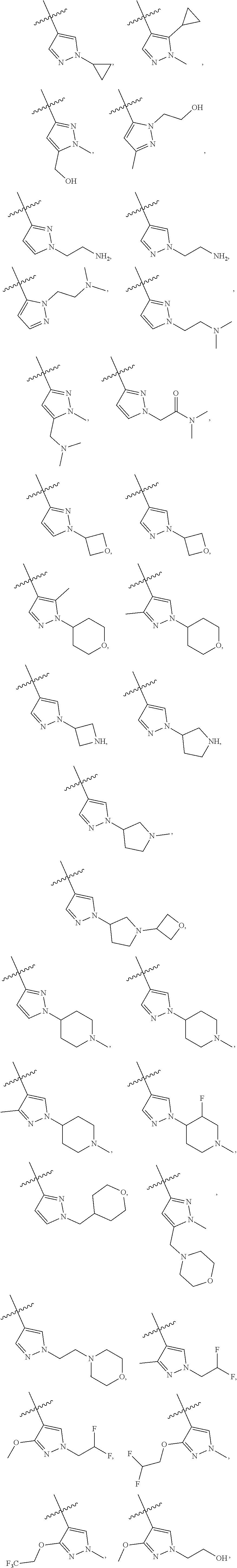

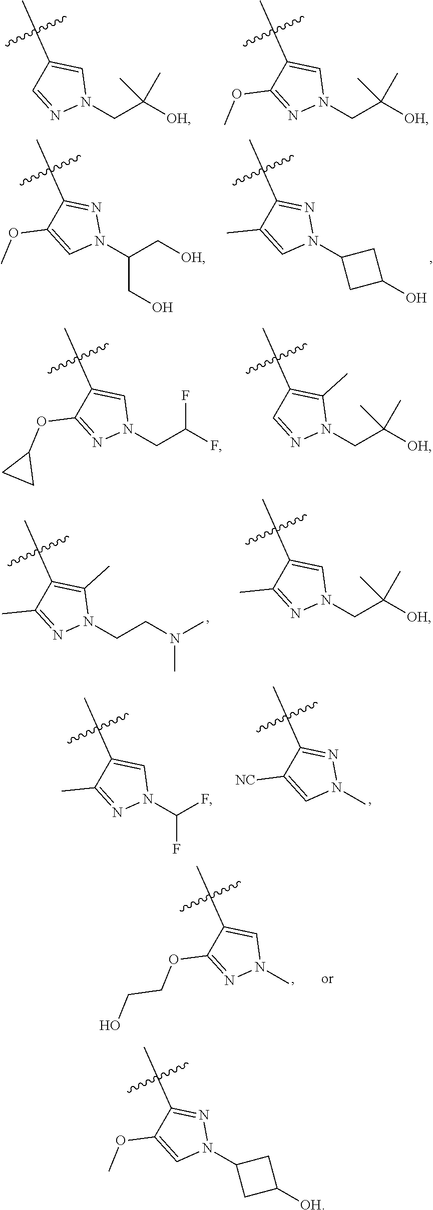

In some embodiments, disclosed herein is a compound of Formula I′ or Formula I or a pharmaceutically acceptable salt thereof, wherein R³ is:

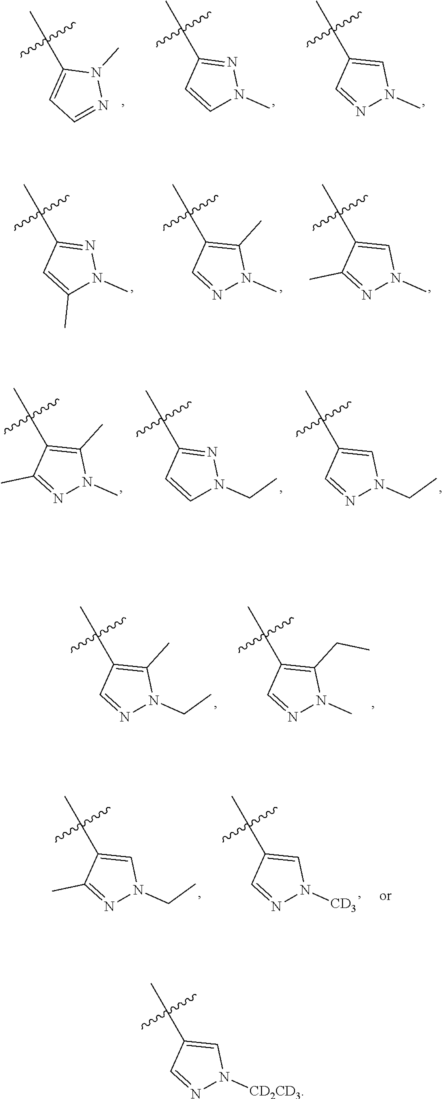

In some embodiments, disclosed herein is a compound of Formula I′ or Formula I or a pharmaceutically acceptable salt thereof, wherein R³ is:

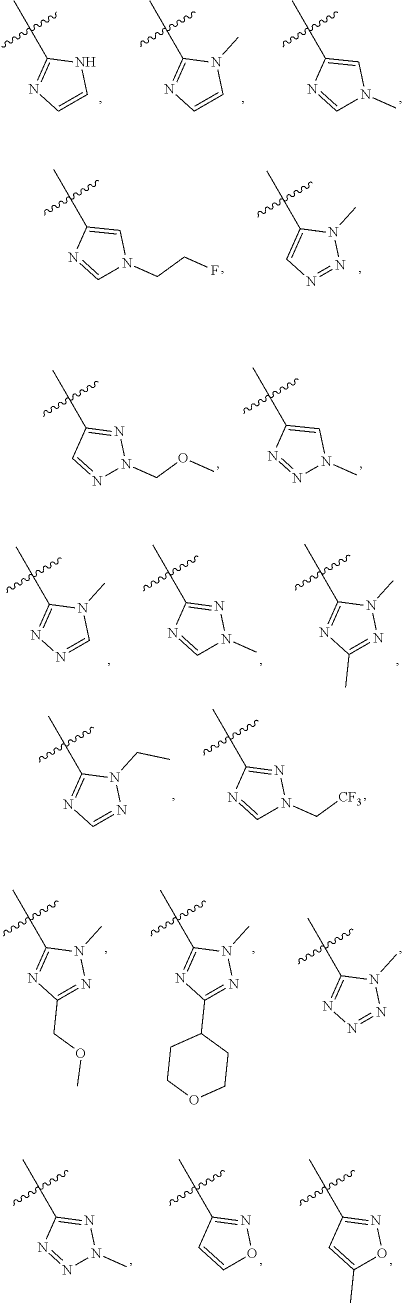

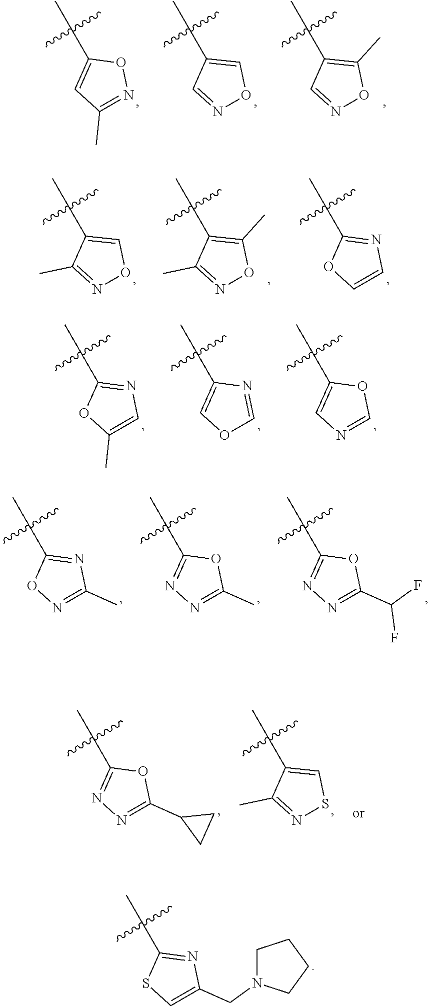

In some embodiments, disclosed herein is a compound of Formula I′, or a pharmaceutically acceptable salt thereof, wherein R³ is:

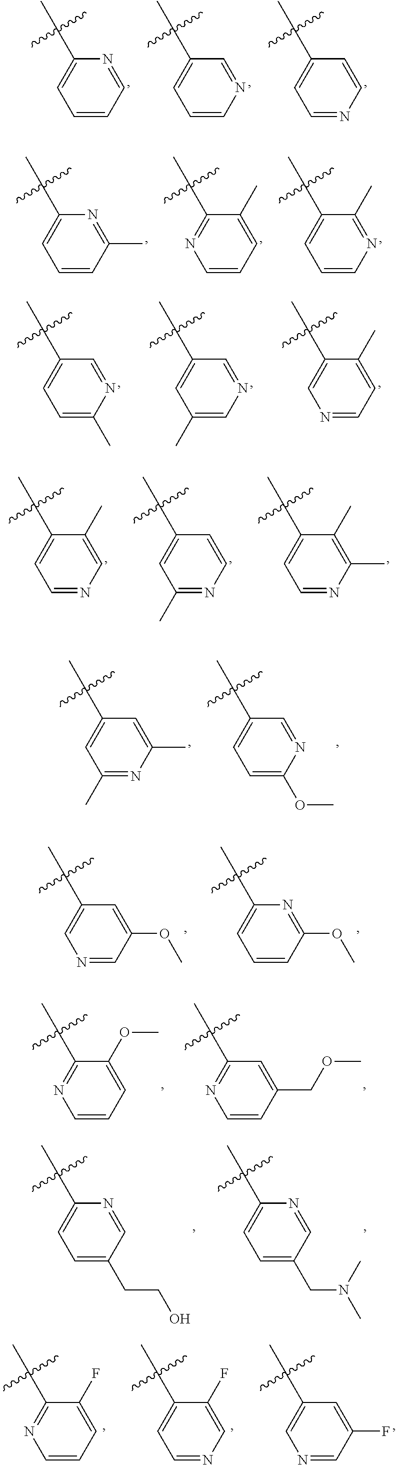

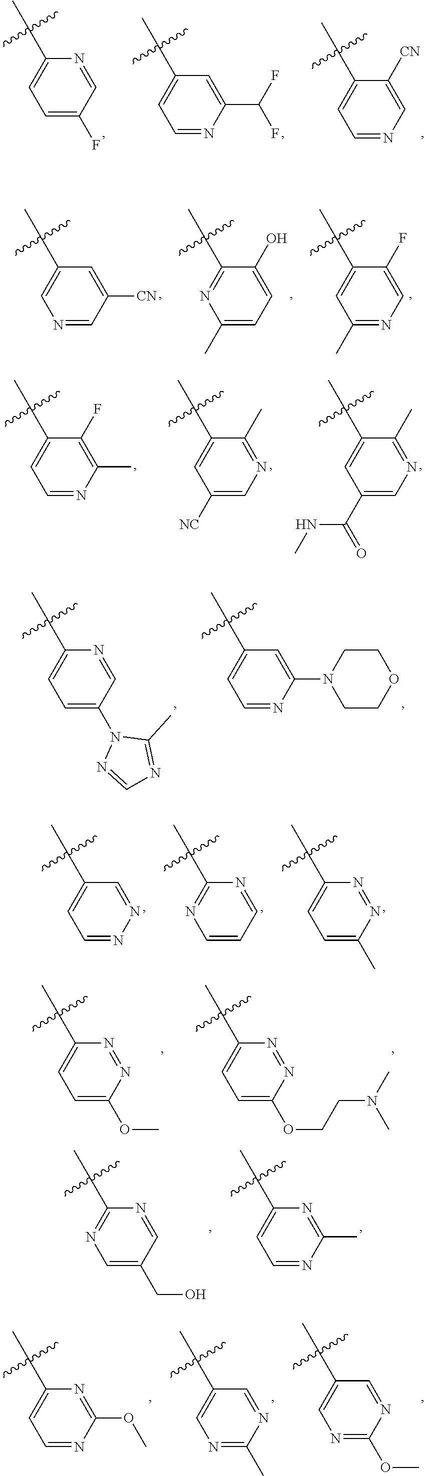

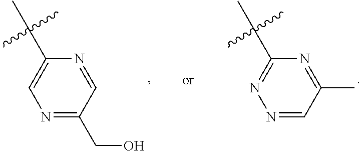

In some embodiments, disclosed herein is a compound of Formula I′ or Formula I, or a pharmaceutically acceptable salt thereof, wherein R³ is:

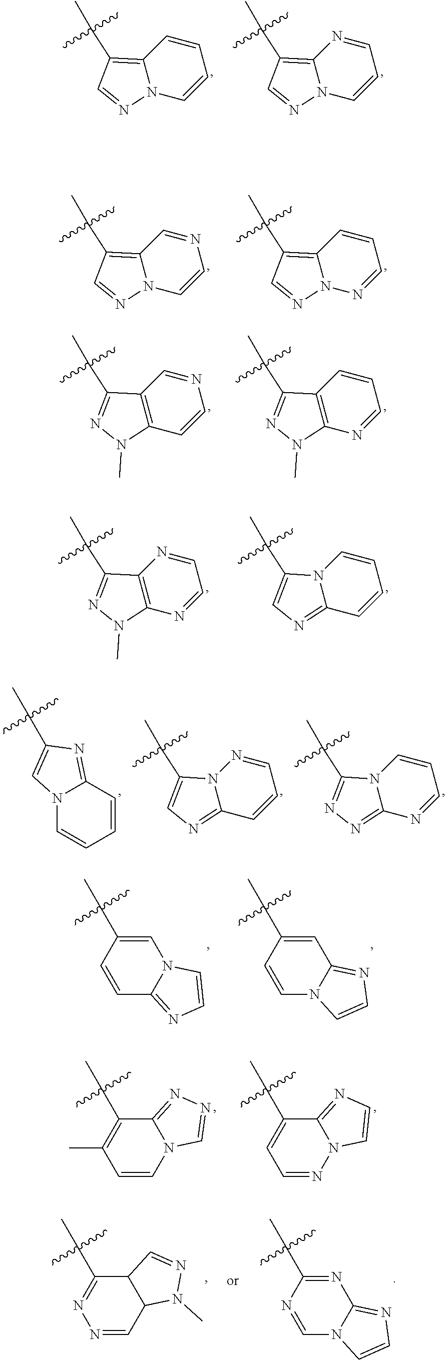

In some embodiments, disclosed herein is a compound of Formula I′ or Formula I or a pharmaceutically acceptable salt thereof, wherein R³ is:

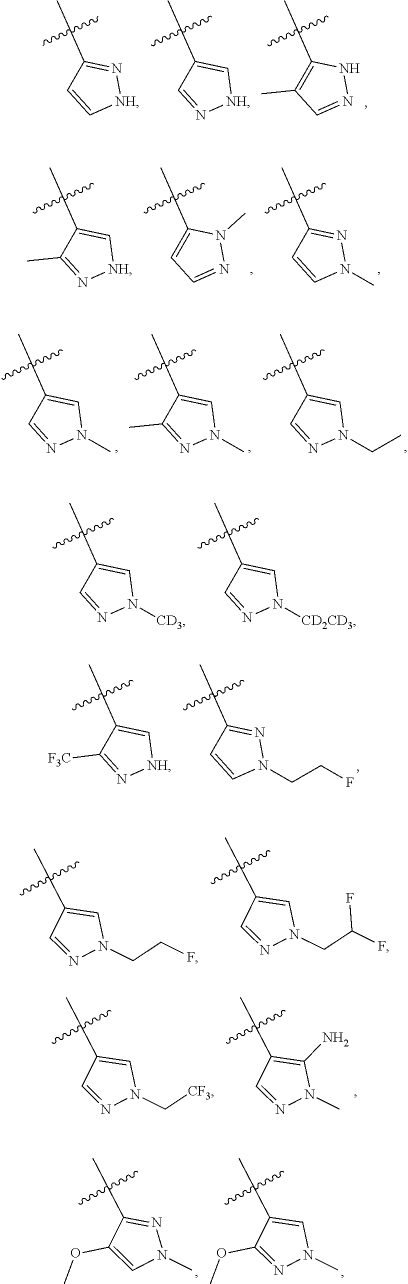

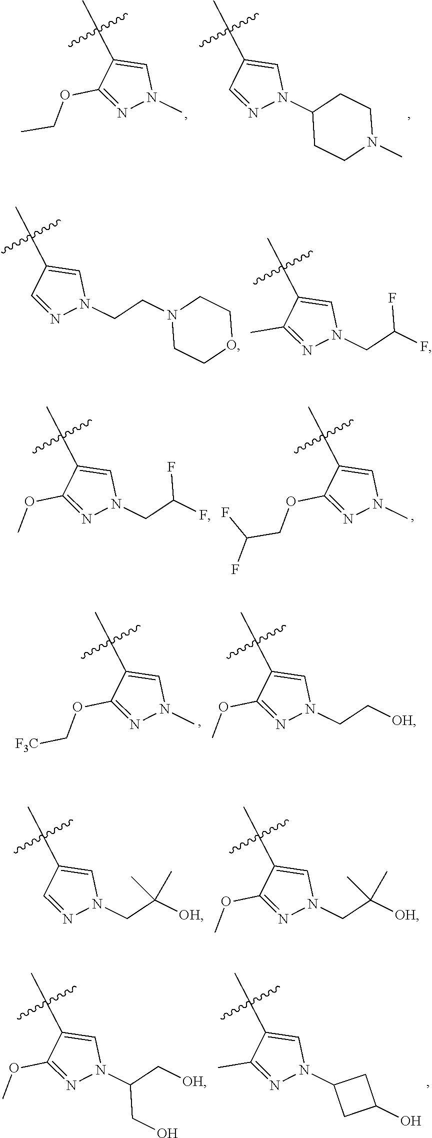

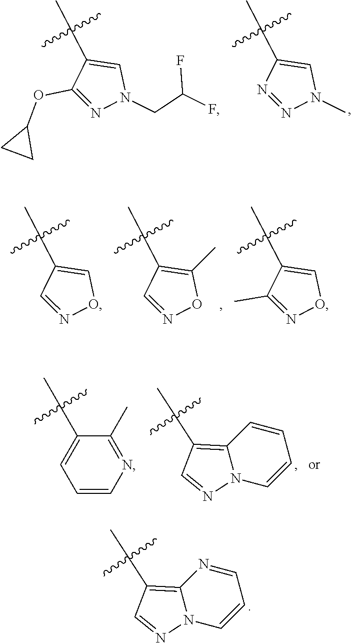

In some embodiments, disclosed herein is a compound of Formula I′ or Formula I, or a pharmaceutically acceptable salt thereof, wherein R³ is:

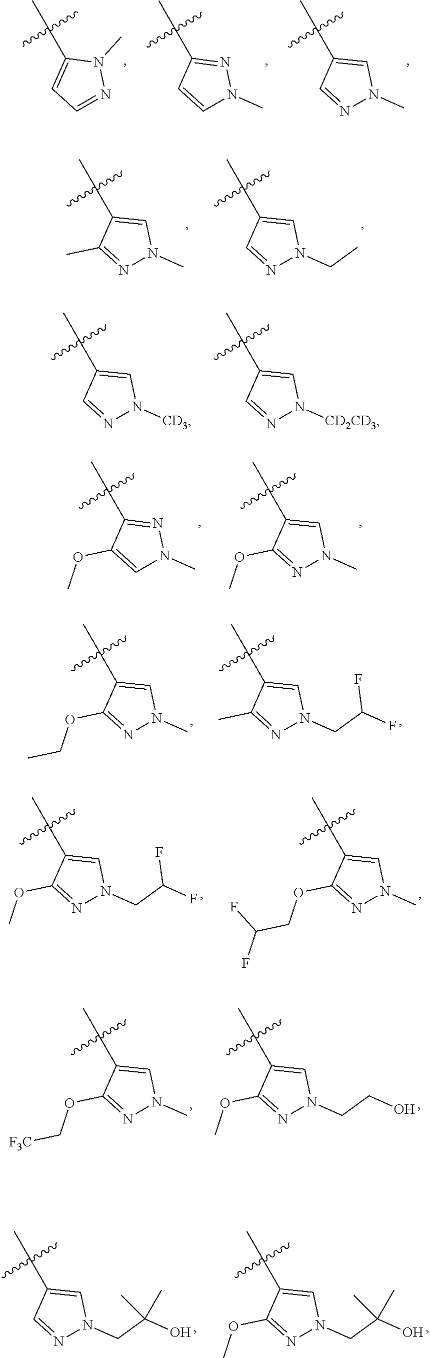

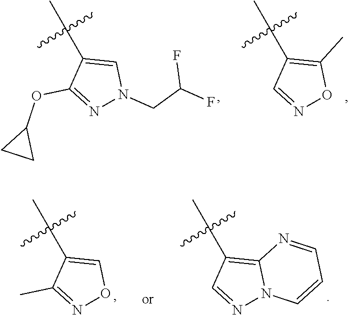

In some embodiments, disclosed herein is a compound of Formula I′ or Formula I, or a pharmaceutically acceptable salt thereof, wherein R³ is:

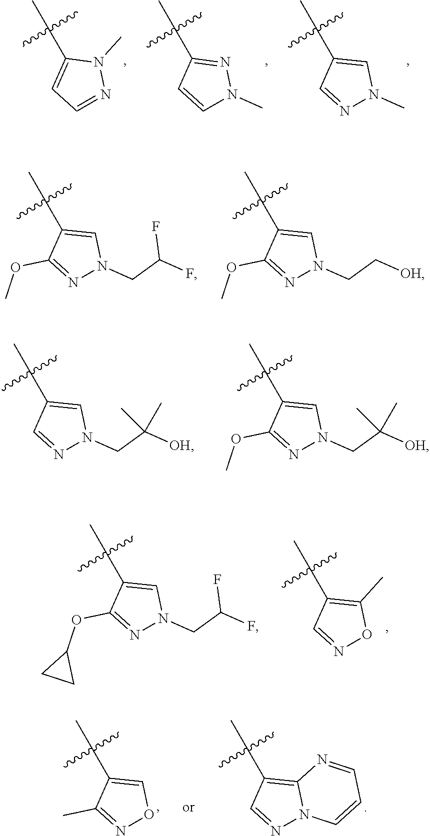

In some embodiments, disclosed herein is a compound of Formula I′ or Formula I, or a pharmaceutically acceptable salt thereof, wherein R³ is:

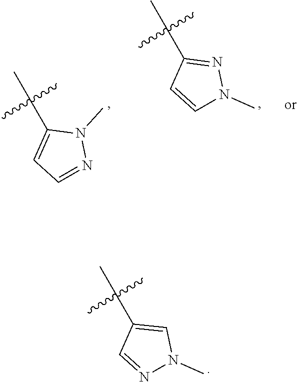

In some embodiments, disclosed herein is a compound of Formula I′ or Formula I, or a pharmaceutically acceptable salt thereof, wherein R³ is

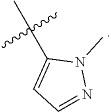 [SMILES: *c1ccnn1C]

In some embodiments, disclosed herein is a compound of Formula I′ or Formula I, or a pharmaceutically acceptable salt thereof, wherein R³ is

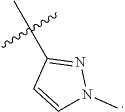 [SMILES: *c1ccn(C)n1]

In some embodiments, disclosed herein is a compound of Formula I′ or Formula I, or a pharmaceutically acceptable salt thereof, wherein R³ is

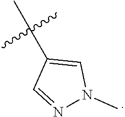 [SMILES: *c1cnn(C)c1]

In some embodiments, disclosed herein is a compound of Formula I′ or Formula I, or a pharmaceutically acceptable salt thereof, wherein R² and L-R³ are combined, along with the nitrogen atom to which they are attached, to form a 3- to 10-membered heterocyclyl, a 5- to 12-membered bicyclic ring system containing one or more heteroatoms, or a 5- to 10-membered heteroaryl, each of which is optionally substituted with one to three groups selected from halo, —OH, —N(R^{N1})(R^{N2}), —CN, —C₍₁₋₆₎alkyl, —C₍₁₋₆₎haloalkyl, —OC₍₁₋₆₎alkyl, and —OC₍₁₋₆₎haloalkyl.

In some embodiments, disclosed herein is a compound of Formula I′ or Formula I, or a pharmaceutically acceptable salt thereof, wherein R² and L-R³ are combined, along with the nitrogen atom to which they are attached, to form a 5- to 12-membered bicyclic ring system containing one or more heteroatoms or a 5- to 10-membered heteroaryl, each of which is optionally substituted with one to three groups selected from halo, —OH, —N(R^{N1})(R^{N2}), —CN, —C₍₁₋₆₎alkyl, —C₍₁₋₆₎haloalkyl, —OC₍₁₋₆₎alkyl and —OC₍₁₋₆₎haloalkyl.

In some embodiments, disclosed herein is a compound of Formula I′ or Formula I, or a pharmaceutically acceptable salt thereof, wherein R² and L-R³ are combined, along with the nitrogen atom to which they are attached, to form a 5- to 12-membered bicyclic ring system containing one or more heteroatoms or a 5- to 10-membered heteroaryl, each of which is optionally substituted with one to three groups selected from —N(R^{N1})(R^{N2}) and —C₍₁₋₆₎alkyl.

In some embodiments, disclosed herein is a compound of Formula I′ or Formula I, or a pharmaceutically acceptable salt thereof, wherein R² and L-R³ are combined, along with the nitrogen atom to which they are attached, to form a 5,6-fused bicyclic ring system containing one or more heteroatoms or a 5-membered heteroaryl, each of which is optionally substituted with one to three groups selected from —N(R^{N1})(R^{N2}) and —C₍₁₋₆₎alkyl

In some embodiments, disclosed herein is a compound of Formula I′ or Formula I, or a pharmaceutically acceptable salt thereof, wherein R² and L-R³ are combined, along with the nitrogen atom to which they are attached, to form:

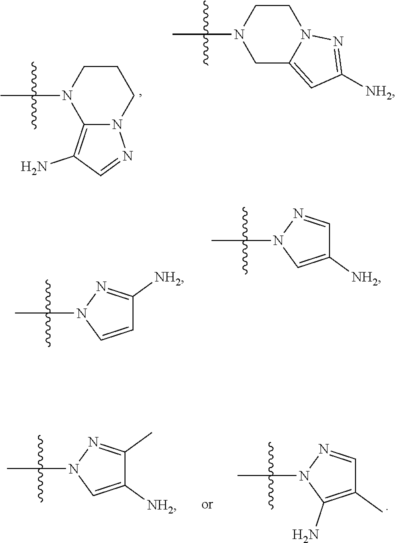

In some embodiments, disclosed herein is a compound of Formula I′ or Formula I, or a pharmaceutically acceptable salt thereof, wherein R² and L-R³ are combined, along with the nitrogen atom to which they are attached, to form:

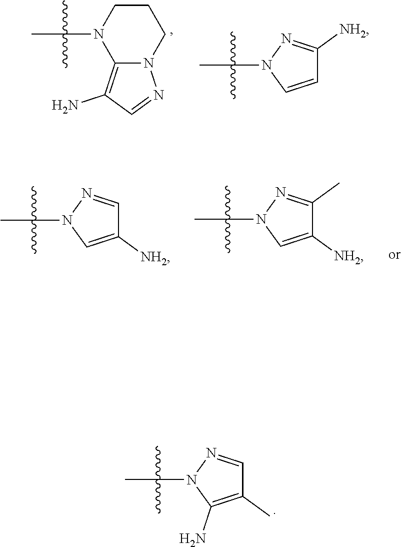

In some embodiments, disclosed herein is a compound of Formula I′, or a pharmaceutically acceptable salt thereof, wherein R³ is —C(O)N(R^{N3})(R^{N4}).

In some embodiments, disclosed herein is a compound of Formula I′, or a pharmaceutically acceptable salt thereof, wherein R^{N3} is hydrogen or —C₍₁₋₄₎alkyl. In some embodiments, disclosed herein is a compound of Formula I′, or a pharmaceutically acceptable salt thereof, wherein R^{N3} is hydrogen or —CH₃. In some embodiments, disclosed herein is a compound of Formula I′, or a pharmaceutically acceptable salt thereof, wherein R^{N3} is hydrogen. In some embodiments, disclosed herein is a compound of Formula I′, or a pharmaceutically acceptable salt thereof, wherein R^{N3} is —C₍₁₋₄₎alkyl. In some embodiments, disclosed herein is a compound of Formula I′, or a pharmaceutically acceptable salt thereof, wherein R^{N3} is —CH₃.

In some embodiments, disclosed herein is a compound of Formula I′, or a pharmaceutically acceptable salt thereof, wherein R^{N4} is hydrogen, —C₍₁₋₄₎alkyl, or phenyl. In some embodiments, disclosed herein is a compound of Formula I′, or a pharmaceutically acceptable salt thereof, wherein R^{N4} is hydrogen, —CH₃, or phenyl. In some embodiments, disclosed herein is a compound of Formula I′, or a pharmaceutically acceptable salt thereof, wherein R^{N4} is hydrogen. In some embodiments, disclosed herein is a compound of Formula I′, or a pharmaceutically acceptable salt thereof, wherein R^{N4} is —C₍₁₋₄₎alkyl. In some embodiments, disclosed herein is a compound of Formula I′, or a pharmaceutically acceptable salt thereof, wherein R^{N4} is —CH₃. In some embodiments, disclosed herein is a compound of Formula I′, or a pharmaceutically acceptable salt thereof, wherein R^{N4} is phenyl.

In some embodiments, disclosed herein is a compound of Formula I′, or a pharmaceutically acceptable salt thereof, wherein R^{N3} and R^{N4} are taken together with the nitrogen atom to which they are attached form a 3- to 10-membered heterocyclyl that is optionally substituted with one to five groups selected from halo, —C₍₁₋₄₎alkyl, and —C₍₁₋₄₎haloalkyl. In some embodiments, disclosed herein is a compound of Formula I′, or a pharmaceutically acceptable salt thereof, wherein R^{N3} and R^{N4} are taken together with the nitrogen atom to which they are attached form a 4- to 6-membered heterocyclyl that is optionally substituted with one to five groups selected from fluorine, —CH₃, and —CF₃.

In some embodiments, disclosed herein is a compound of Formula I′, or a pharmaceutically acceptable salt thereof, wherein R³ is:

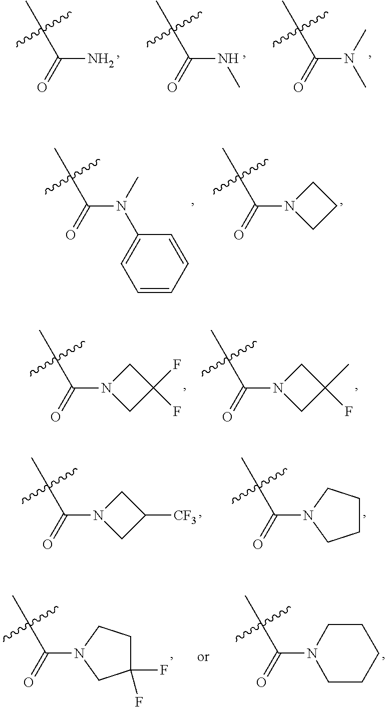

In some embodiments, disclosed herein is a compound of Formula I′ or Formula I, or a pharmaceutically acceptable salt thereof, wherein R^{N1} and R^{N2} are each independently for each occurrence H or —C₍₁₋₃₎alkyl. In some embodiments, disclosed herein is a compound of Formula I′ or Formula I, or a pharmaceutically acceptable salt thereof, wherein R^{N1} and R^{N2} are each hydrogen.

In some embodiments, disclosed herein is a compound of Formula I′, or a pharmaceutically acceptable salt thereof, wherein R⁴ is hydrogen, chlorine, fluorine, or —CH₃. In some embodiments, disclosed herein is a compound of Formula I′, or a pharmaceutically acceptable salt thereof, wherein R⁴ is hydrogen. In some embodiments, disclosed herein is a compound of Formula I′, or a pharmaceutically acceptable salt thereof, wherein R⁴ is protium. In some embodiments, disclosed herein is a compound of Formula I′, or a pharmaceutically acceptable salt thereof, wherein R⁴ is deuterium. In some embodiments, disclosed herein is a compound of Formula I′, or a pharmaceutically acceptable salt thereof, wherein R⁴ is chlorine. In some embodiments, disclosed herein is a compound of Formula I′, or a pharmaceutically acceptable salt thereof, wherein R⁴ is fluorine. In some embodiments, disclosed herein is a compound of Formula I′, or a pharmaceutically acceptable salt thereof, wherein R⁴ is —CH₃.

In some embodiments, disclosed herein is a compound of Formula I′, or a pharmaceutically acceptable salt thereof, wherein R⁵ is hydrogen, chlorine, fluorine, or —CH₃. In some embodiments, disclosed herein is a compound of Formula I′, or a pharmaceutically acceptable salt thereof, wherein R⁵ is hydrogen. In some embodiments, disclosed herein is a compound of Formula I′, or a pharmaceutically acceptable salt thereof, wherein R⁵ is protium. In some embodiments, disclosed herein is a compound of Formula I′, or a pharmaceutically acceptable salt thereof, wherein R⁵ is deuterium. In some embodiments, disclosed herein is a compound of Formula I′, or a pharmaceutically acceptable salt thereof, wherein R⁵ is chlorine. In some embodiments, disclosed herein is a compound of Formula I′, or a pharmaceutically acceptable salt thereof, wherein R⁵ is fluorine. In some embodiments, disclosed herein is a compound of Formula I′, or a pharmaceutically acceptable salt thereof, wherein R⁵ is —CH₃.

In some embodiments, disclosed herein is a compound of Formula I′, or a pharmaceutically acceptable salt thereof, wherein R⁴ is hydrogen, and R⁵ is hydrogen; R⁴ is fluorine, and R⁵ is hydrogen; R⁴ is —CH₃; and R⁵ is hydrogen; R⁴ is hydrogen, and R⁵ is chlorine; or R⁴ is hydrogen, and R⁵ is —CH₃.

In some embodiments, disclosed herein is a compound of Formula I′ or Formula I, or a pharmaceutically acceptable salt thereof, which is a compound of Formula Ih:

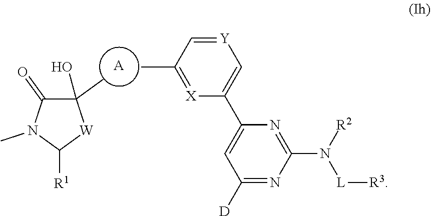

In some embodiments, disclosed herein is a compound of Formula I′ or Formula I, or a pharmaceutically acceptable salt thereof, which is a compound of Formula Ih-1:

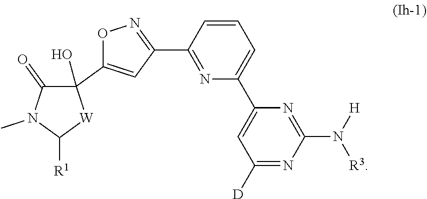

In some embodiments, disclosed herein is a compound of Formula I′ or Formula I, or a pharmaceutically acceptable salt thereof, which is a compound of Formula Ih-2:

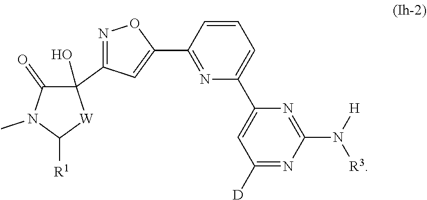

In some embodiments, disclosed herein is a compound of Formula I′ or Formula I or a pharmaceutically acceptable salt thereof, which is a compound of Formula Ih-3:

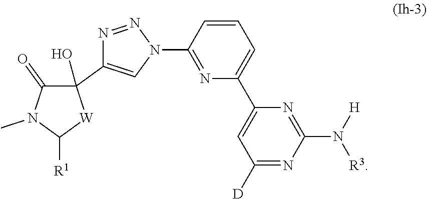

In some embodiments, disclosed herein is a compound of Formula I′ or Formula I or a pharmaceutically acceptable salt thereof, which is a compound of Formula Ig-1:

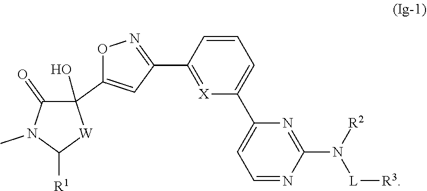

In some embodiments, disclosed herein is a compound of Formula I′ or Formula I or a pharmaceutically acceptable salt thereof, which is a compound of Formula Ig-2:

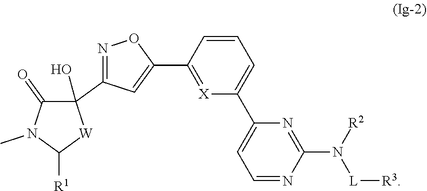

In some embodiments, disclosed herein is a compound of Formula I′ or Formula I or a pharmaceutically acceptable salt thereof, which is a compound of Formula Ig-1:

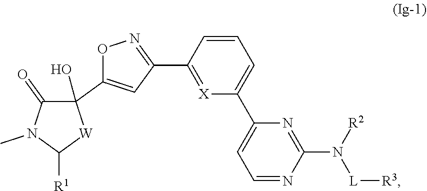

wherein:
W is CH₂ or CF₂;
X is N or C—H; R¹ is hydrogen; R² is hydrogen; L is absent or —C₍₁₋₄₎alkylene; R³ is 5- to 10-membered heteroaryl, which is optionally substituted with one to five R^{3e} groups;
each R^{3e} is independently —C₍₁₋₆₎alkyl, —OC₍₃₋₈₎cycloalkyl, —OC₍₁₋₆₎alkyl, or —OC₍₃₋₈₎cycloalkyl, each of which is optionally further substituted with one to five groups selected from fluorine and —OH.

In some embodiments, disclosed herein is a compound of Formula I′, or a pharmaceutically acceptable salt thereof, which is a compound of Formula Ii:

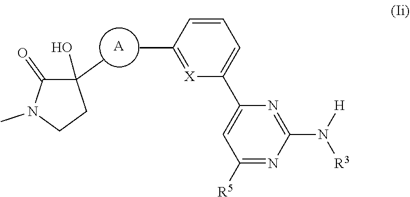

wherein: A is triazolyl or isoxazolyl;
X is N or C—H; R³ is 5-membered heteroaryl, which is optionally substituted with one to five R^{3e} groups;
each R^{3e} is independently —C₍₁₋₆₎alkyl, —OC₍₃₋₈₎cycloalkyl, —OC₍₁₋₆₎alkyl, or —OC₍₃₋₈₎cycloalkyl, each of which is optionally further substituted with one to five groups selected from fluorine and —OH; R⁵ is protium (¹H) or deuterium (²H).

In some embodiments, disclosed herein is a compound of Formula I′ or Formula I, or a pharmaceutically acceptable salt thereof, having a structure as shown in any one of Tables 1A to 1D.

TABLE 1A

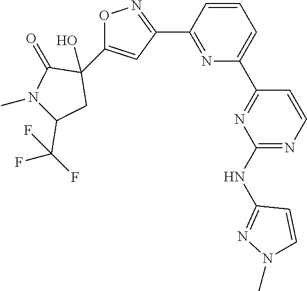 [SMILES: CN1C(=O)C(O)(c2cc(-c3cccc(-c4ccnc(Nc5ccn(C)n5)n4)n3)no2)CC1C(F)(F)F]

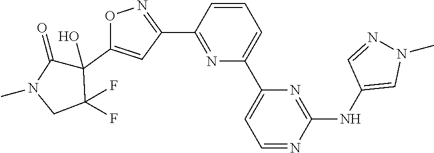

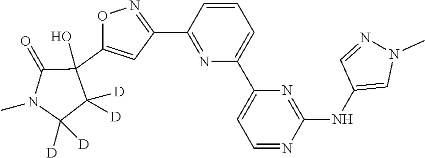

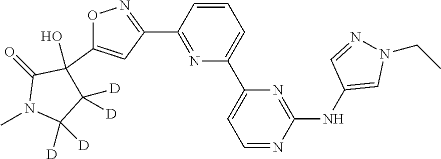 [SMILES: [2H]C1([2H])N(C)C(=O)C(O)(c2cc(-c3cccc(-c4ccnc(Nc5cnn(CC)c5)n4)n3)no2)C1([2H])[2H]]

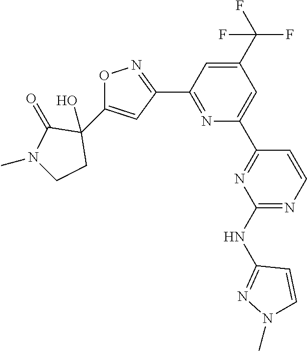 [SMILES: CN1CCC(O)(c2cc(-c3cc(C(F)(F)F)cc(-c4ccnc(Nc5ccn(C)n5)n4)n3)no2)C1=O]

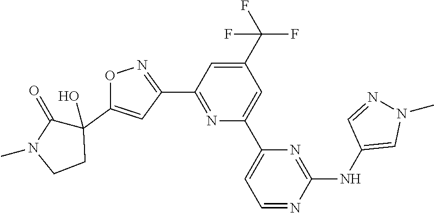

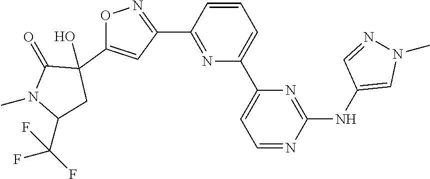

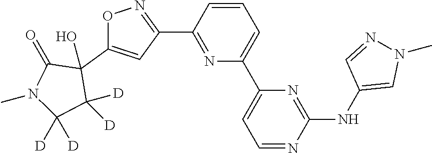

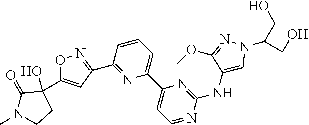 [SMILES: COc1nn(C(CO)CO)cc1Nc1nccc(-c2cccc(-c3cc(C4(O)CCN(C)C4=O)on3)n2)n1]

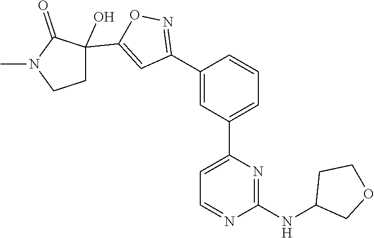 [SMILES: CN1CCC(O)(c2cc(-c3cccc(-c4ccnc(NC5CCOC5)n4)c3)no2)C1=O]

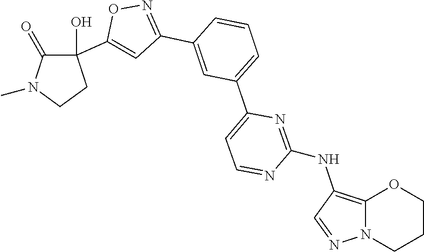 [SMILES: CN1CCC(O)(c2cc(-c3cccc(-c4ccnc(Nc5cnn6c5OCCC6)n4)c3)no2)C1=O]

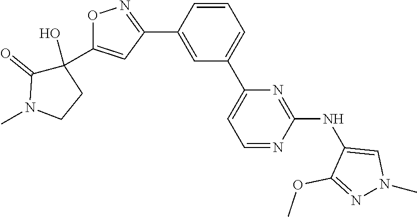

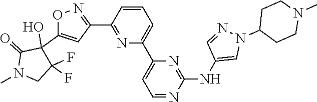

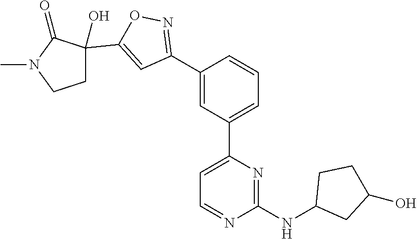 [SMILES: CN1CCC(O)(c2cc(-c3cccc(-c4ccnc(NC5CCC(O)C5)n4)c3)no2)C1=O]

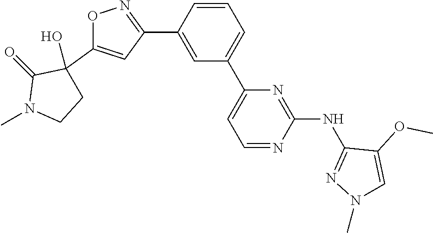

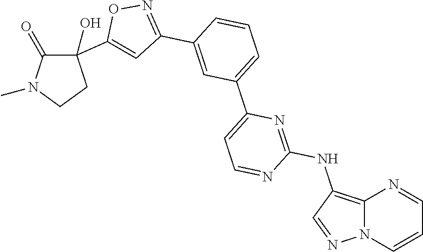 [SMILES: CN1CCC(O)(c2cc(-c3cccc(-c4ccnc(Nc5cnn6cccnc56)n4)c3)no2)C1=O]

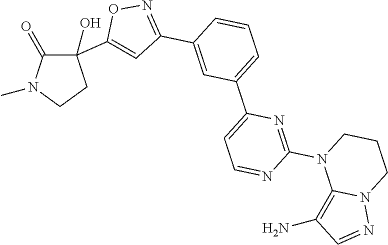 [SMILES: CN1CCC(O)(c2cc(-c3cccc(-c4ccnc(N5CCCn6ncc(N)c65)n4)c3)no2)C1=O]

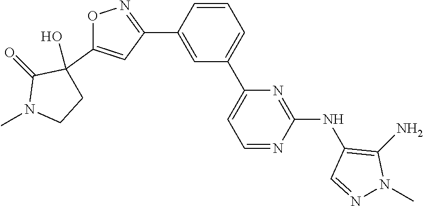

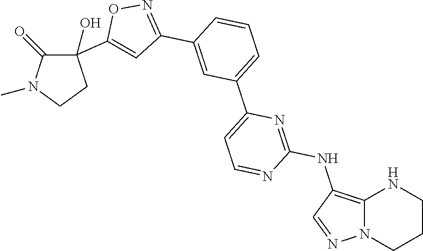 [SMILES: CN1CCC(O)(c2cc(-c3cccc(-c4ccnc(Nc5cnn6c5NCCC6)n4)c3)no2)C1=O]

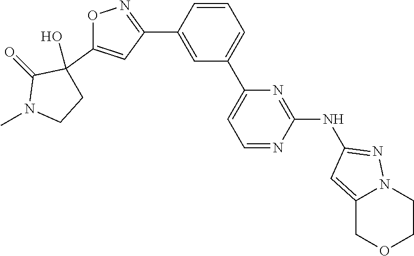 [SMILES: CN1CCC(O)(c2cc(-c3cccc(-c4ccnc(Nc5cc6n(n5)CCOC6)n4)c3)no2)C1=O]

TABLE 1B

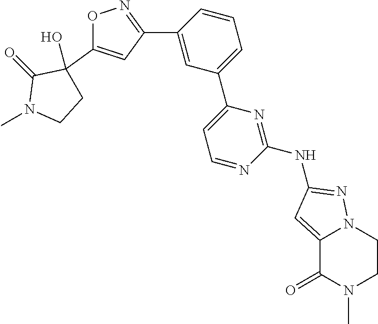 [SMILES: CN1CCn2nc(Nc3nccc(-c4cccc(-c5cc(C6(O)CCN(C)C6=O)on5)c4)n3)cc2C1=O]

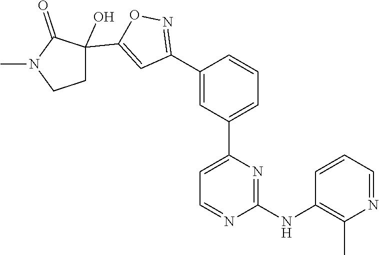 [SMILES: Cc1ncccc1Nc1nccc(-c2cccc(-c3cc(C4(O)CCN(C)C4=O)on3)c2)n1]

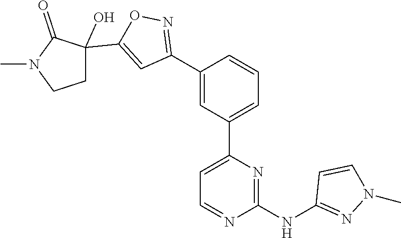

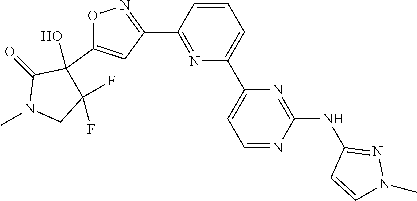

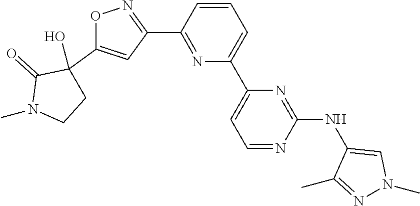

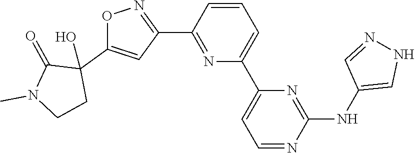 [SMILES: CN1CCC(O)(c2cc(-c3cccc(-c4ccnc(Nc5cn[nH]c5)n4)n3)no2)C1=O]

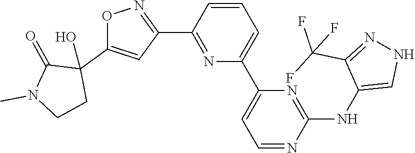 [SMILES: CN1CCC(O)(c2cc(-c3cccc(-c4ccnc(Nc5c[nH]nc5C(F)(F)F)n4)n3)no2)C1=O]

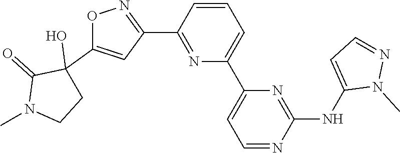

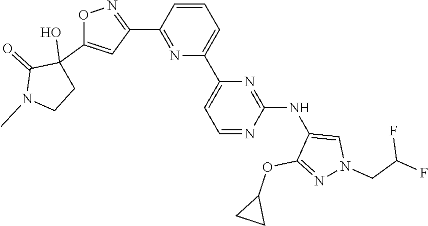 [SMILES: CN1CCC(O)(c2cc(-c3cccc(-c4ccnc(Nc5cn(CC(F)F)nc5OC5CC5)n4)n3)no2)C1=O]

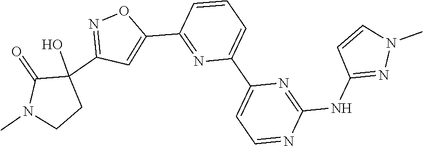

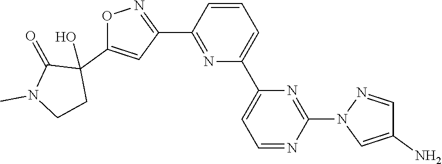 [SMILES: CN1CCC(O)(c2cc(-c3cccc(-c4ccnc(-n5cc(N)cn5)n4)n3)no2)C1=O]

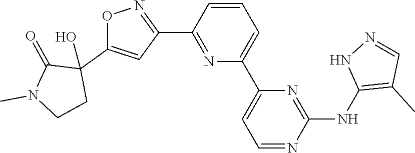

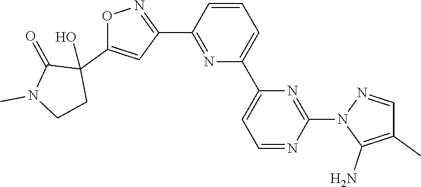

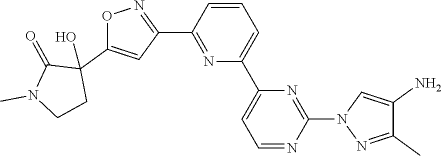 [SMILES: Cc1nn(-c2nccc(-c3cccc(-c4cc(C5(O)CCN(C)C5=O)on4)n3)n2)cc1N]

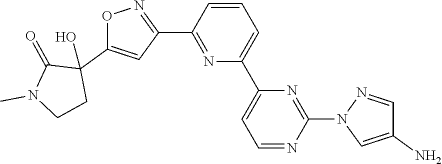 [SMILES: CN1CCC(O)(c2cc(-c3cccc(-c4ccnc(-n5cc(N)cn5)n4)n3)no2)C1=O]

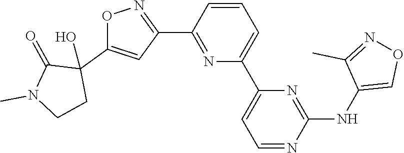 [SMILES: Cc1nocc1Nc1nccc(-c2cccc(-c3cc(C4(O)CCN(C)C4=O)on3)n2)n1]

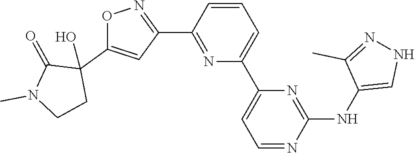 [SMILES: Cc1n[nH]cc1Nc1nccc(-c2cccc(-c3cc(C4(O)CCN(C)C4=O)on3)n2)n1]

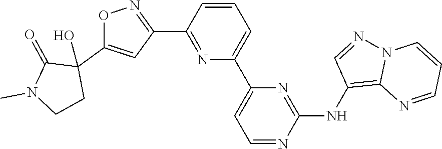 [SMILES: CN1CCC(O)(c2cc(-c3cccc(-c4ccnc(Nc5cnn6cccnc56)n4)n3)no2)C1=O]

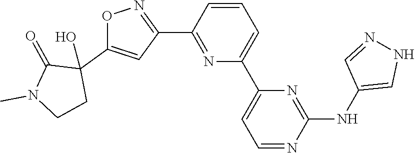 [SMILES: CN1CCC(O)(c2cc(-c3cccc(-c4ccnc(Nc5cn[nH]c5)n4)n3)no2)C1=O]

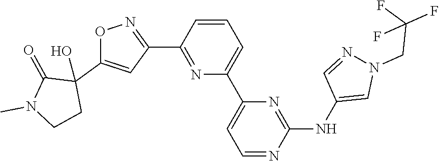 [SMILES: CN1CCC(O)(c2cc(-c3cccc(-c4ccnc(Nc5cnn(CC(F)(F)F)c5)n4)n3)no2)C1=O]

TABLE 1C

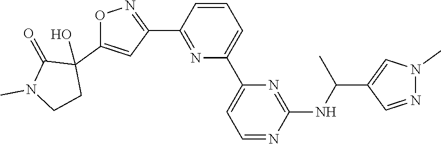

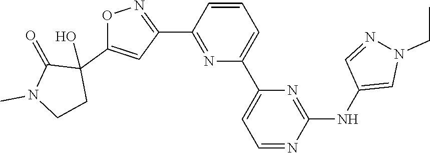 [SMILES: CCn1cc(Nc2nccc(-c3cccc(-c4cc(C5(O)CCN(C)C5=O)on4)n3)n2)cn1]

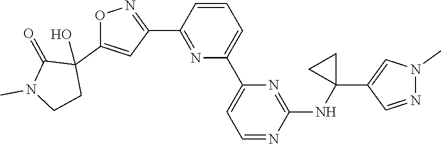

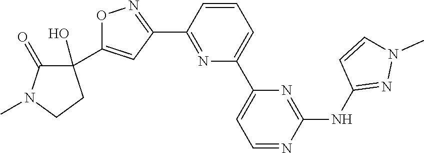

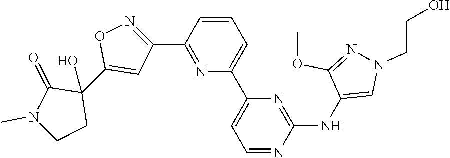 [SMILES: COc1nn(CCO)cc1Nc1nccc(-c2cccc(-c3cc(C4(O)CCN(C)C4=O)on3)n2)n1]

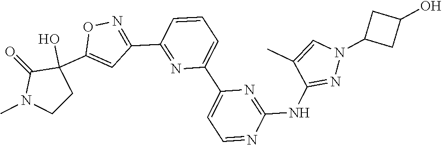 [SMILES: Cc1cn(C2CC(O)C2)nc1Nc1nccc(-c2cccc(-c3cc(C4(O)CCN(C)C4=O)on3)n2)n1]

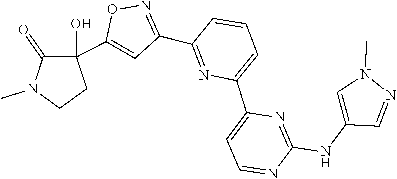 [SMILES: CN1CCC(O)(c2cc(-c3cccc(-c4ccnc(Nc5cnn(C)c5)n4)n3)no2)C1=O]

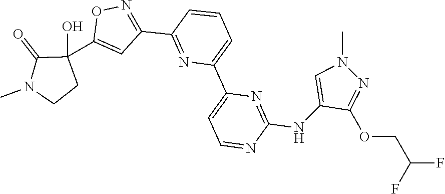 [SMILES: CN1CCC(O)(c2cc(-c3cccc(-c4ccnc(Nc5cn(C)nc5OCC(F)F)n4)n3)no2)C1=O]

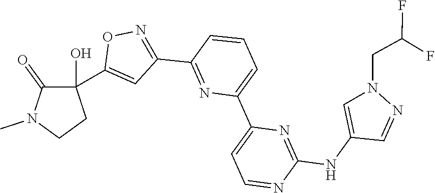 [SMILES: CN1CCC(O)(c2cc(-c3cccc(-c4ccnc(Nc5cnn(CC(F)F)c5)n4)n3)no2)C1=O]

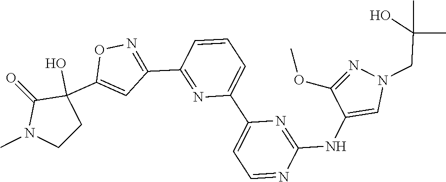 [SMILES: COc1nn(CC(C)(C)O)cc1Nc1nccc(-c2cccc(-c3cc(C4(O)CCN(C)C4=O)on3)n2)n1]

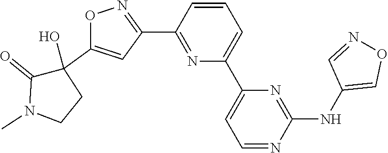 [SMILES: CN1CCC(O)(c2cc(-c3cccc(-c4ccnc(Nc5cnoc5)n4)n3)no2)C1=O]

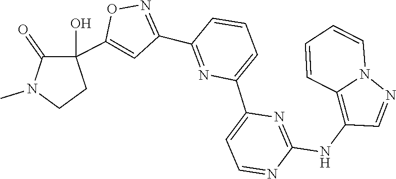 [SMILES: CN1CCC(O)(c2cc(-c3cccc(-c4ccnc(Nc5cnn6ccccc56)n4)n3)no2)C1=O]

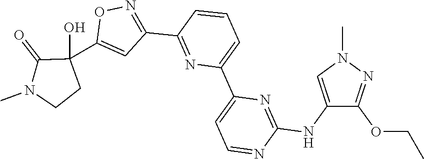 [SMILES: CCOc1nn(C)cc1Nc1nccc(-c2cccc(-c3cc(C4(O)CCN(C)C4=O)on3)n2)n1]

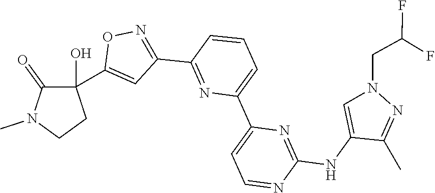 [SMILES: Cc1nn(CC(F)F)cc1Nc1nccc(-c2cccc(-c3cc(C4(O)CCN(C)C4=O)on3)n2)n1]

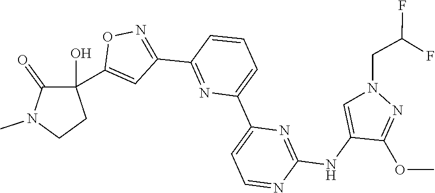

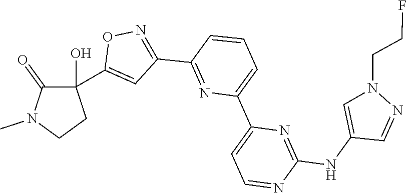 [SMILES: CN1CCC(O)(c2cc(-c3cccc(-c4ccnc(Nc5cnn(CCF)c5)n4)n3)no2)C1=O]

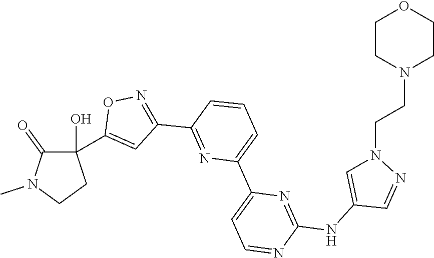 [SMILES: CN1CCC(O)(c2cc(-c3cccc(-c4ccnc(Nc5cnn(CCN6CCOCC6)c5)n4)n3)no2)C1=O]

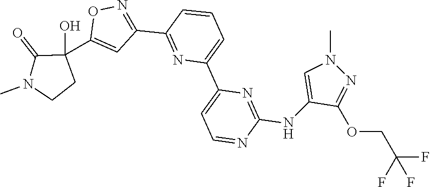 [SMILES: CN1CCC(O)(c2cc(-c3cccc(-c4ccnc(Nc5cn(C)nc5OCC(F)(F)F)n4)n3)no2)C1=O]

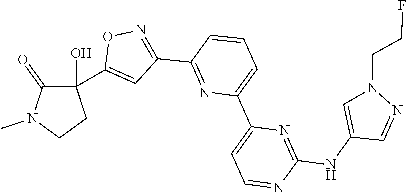 [SMILES: CN1CCC(O)(c2cc(-c3cccc(-c4ccnc(Nc5cnn(CCF)c5)n4)n3)no2)C1=O]

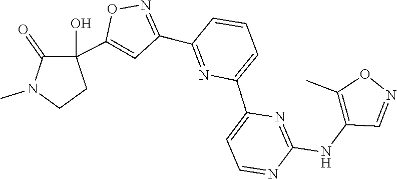 [SMILES: Cc1oncc1Nc1nccc(-c2cccc(-c3cc(C4(O)CCN(C)C4=O)on3)n2)n1]

TABLE 1D

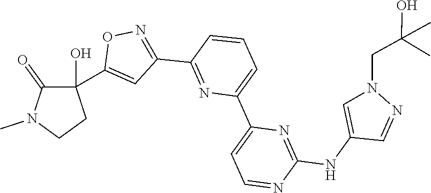 [SMILES: CN1CCC(O)(c2cc(-c3cccc(-c4ccnc(Nc5cnn(CC(C)(C)O)c5)n4)n3)no2)C1=O]

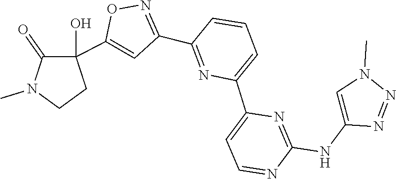 [SMILES: CN1CCC(O)(c2cc(-c3cccc(-c4ccnc(Nc5cn(C)nn5)n4)n3)no2)C1=O]

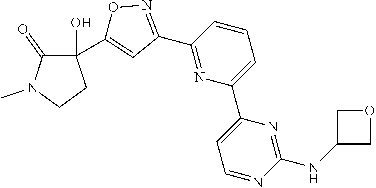 [SMILES: CN1CCC(O)(c2cc(-c3cccc(-c4ccnc(NC5COC5)n4)n3)no2)C1=O]

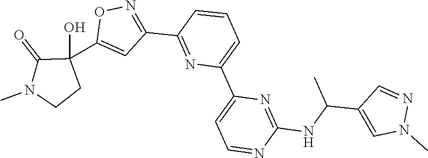

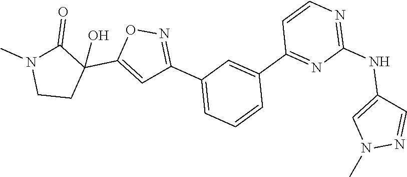 [SMILES: CN1CCC(O)(c2cc(-c3cccc(-c4ccnc(Nc5cnn(C)c5)n4)c3)no2)C1=O]

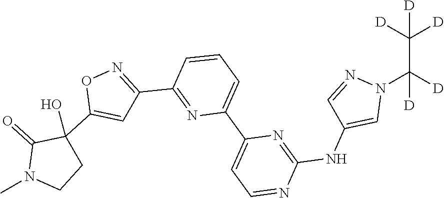 [SMILES: [2H]C([2H])([2H])C([2H])([2H])n1cc(Nc2nccc(-c3cccc(-c4cc(C5(O)CCN(C)C5=O)on4)n3)n2)cn1]

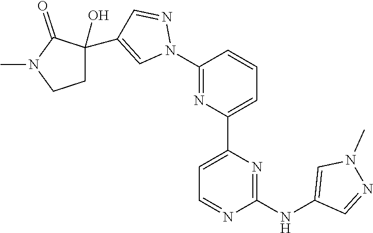 [SMILES: CN1CCC(O)(c2cnn(-c3cccc(-c4ccnc(Nc5cnn(C)c5)n4)n3)c2)C1=O]

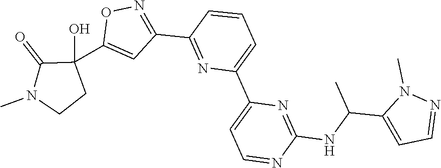 [SMILES: CC(Nc1nccc(-c2cccc(-c3cc(C4(O)CCN(C)C4=O)on3)n2)n1)c1ccnn1C]

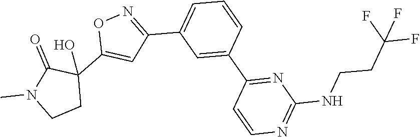 [SMILES: CN1CCC(O)(c2cc(-c3cccc(-c4ccnc(NCCC(F)(F)F)n4)c3)no2)C1=O]

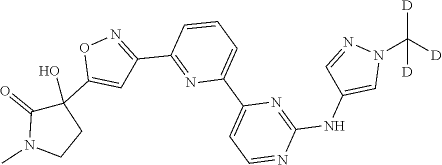 [SMILES: [2H]C([2H])([2H])n1cc(Nc2nccc(-c3cccc(-c4cc(C5(O)CCN(C)C5=O)on4)n3)n2)cn1]

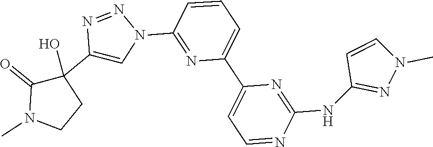

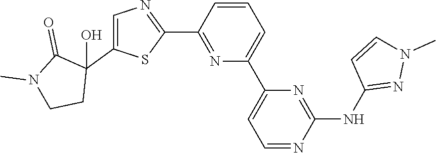

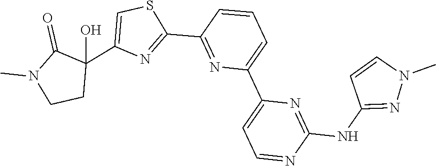

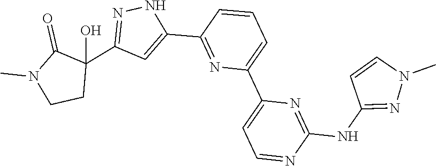

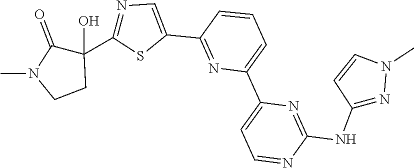

In some embodiments, disclosed herein is a compound, or a pharmaceutically acceptable salt thereof, having a structure as shown in any one of Tables 1E to 1U.

TABLE 1E

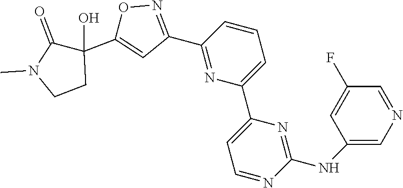 [SMILES: CN1CCC(O)(c2cc(-c3cccc(-c4ccnc(Nc5cncc(F)c5)n4)n3)no2)C1=O]

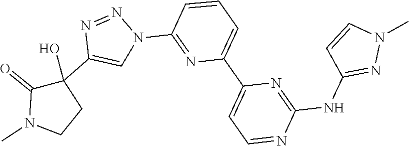

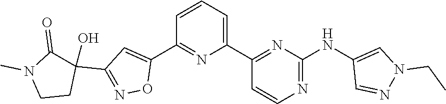 [SMILES: CCn1cc(Nc2nccc(-c3cccc(-c4cc(C5(O)CCN(C)C5=O)no4)n3)n2)cn1]

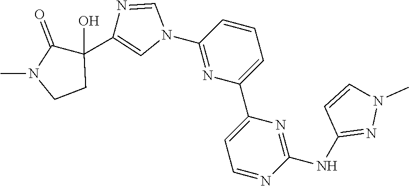

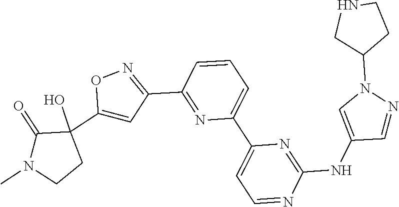 [SMILES: CN1CCC(O)(c2cc(-c3cccc(-c4ccnc(Nc5cnn(C6CCNC6)c5)n4)n3)no2)C1=O]

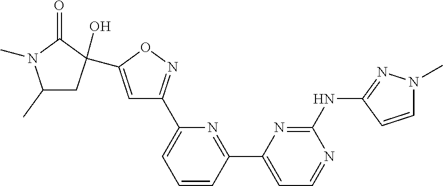

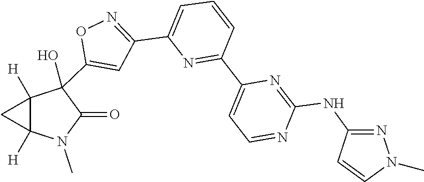 [SMILES: CN1C(=O)C(O)(c2cc(-c3cccc(-c4ccnc(Nc5ccn(C)n5)n4)n3)no2)C2CC21]

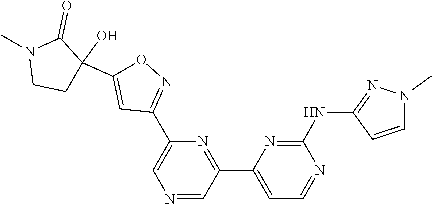

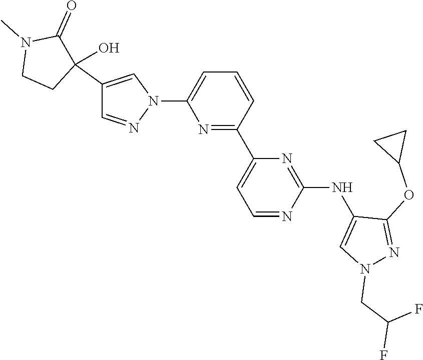 [SMILES: CN1CCC(O)(c2cnn(-c3cccc(-c4ccnc(Nc5cn(CC(F)F)nc5OC5CC5)n4)n3)c2)C1=O]

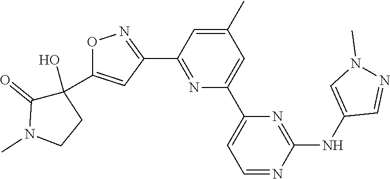 [SMILES: Cc1cc(-c2cc(C3(O)CCN(C)C3=O)on2)nc(-c2ccnc(Nc3cnn(C)c3)n2)c1]

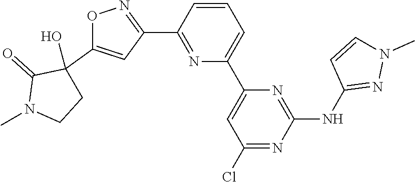

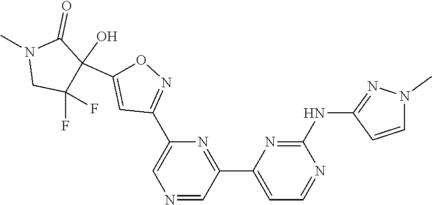

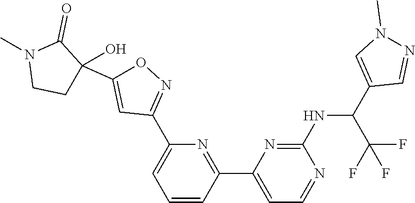 [SMILES: CN1CCC(O)(c2cc(-c3cccc(-c4ccnc(NC(c5cnn(C)c5)C(F)(F)F)n4)n3)no2)C1=O]

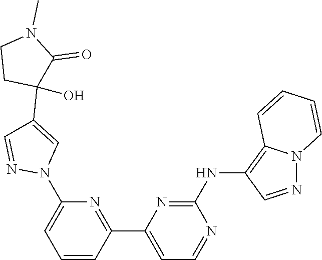 [SMILES: CN1CCC(O)(c2cnn(-c3cccc(-c4ccnc(Nc5cnn6ccccc56)n4)n3)c2)C1=O]

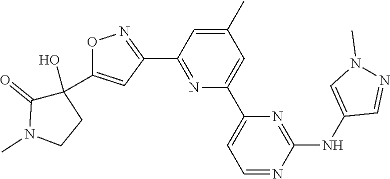 [SMILES: Cc1cc(-c2cc(C3(O)CCN(C)C3=O)on2)nc(-c2ccnc(Nc3cnn(C)c3)n2)c1]

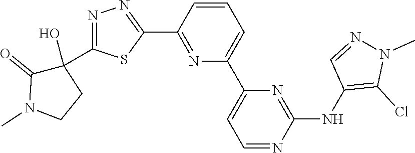

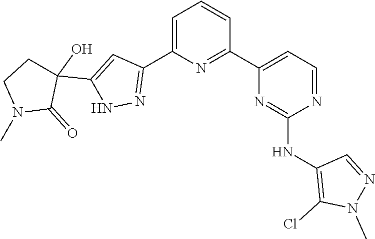 [SMILES: CN1CCC(O)(c2cc(-c3cccc(-c4ccnc(Nc5cnn(C)c5Cl)n4)n3)n[nH]2)C1=O]

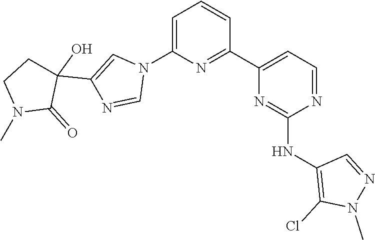 [SMILES: CN1CCC(O)(c2cn(-c3cccc(-c4ccnc(Nc5cnn(C)c5Cl)n4)n3)cn2)C1=O]

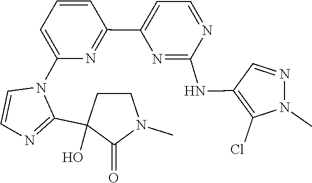 [SMILES: CN1CCC(O)(c2nccn2-c2cccc(-c3ccnc(Nc4cnn(C)c4Cl)n3)n2)C1=O]

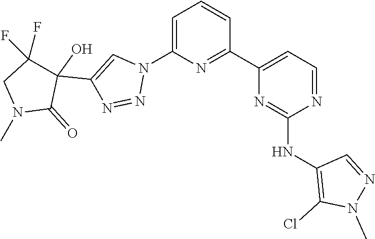 [SMILES: CN1CC(F)(F)C(O)(c2cn(-c3cccc(-c4ccnc(Nc5cnn(C)c5Cl)n4)n3)nn2)C1=O]

TABLE 1F

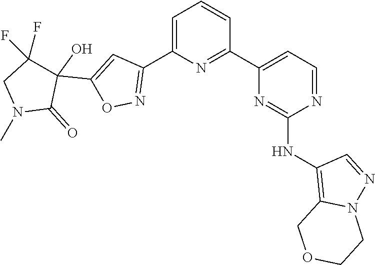 [SMILES: CN1CC(F)(F)C(O)(c2cc(-c3cccc(-c4ccnc(Nc5cnn6c5COCC6)n4)n3)no2)C1=O]

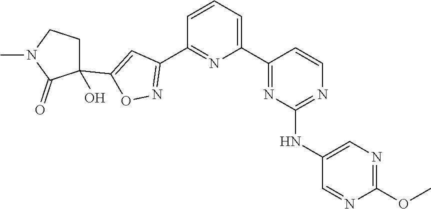

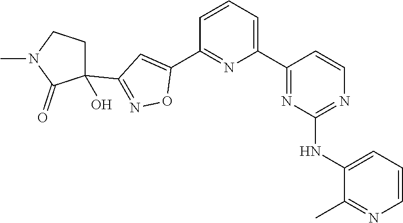 [SMILES: Cc1ncccc1Nc1nccc(-c2cccc(-c3cc(C4(O)CCN(C)C4=O)no3)n2)n1]

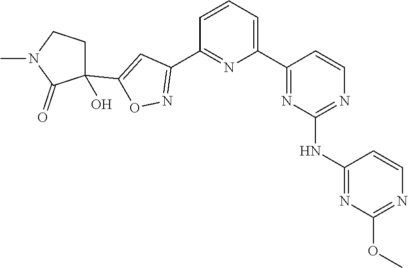 [SMILES: COc1nccc(Nc2nccc(-c3cccc(-c4cc(C5(O)CCN(C)C5=O)on4)n3)n2)n1]

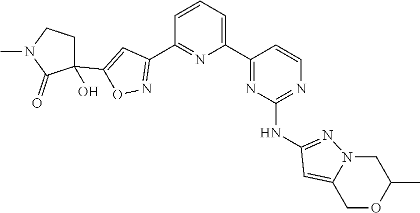

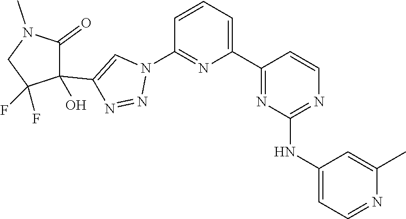 [SMILES: Cc1cc(Nc2nccc(-c3cccc(-n4cc(C5(O)C(=O)N(C)CC5(F)F)nn4)n3)n2)ccn1]

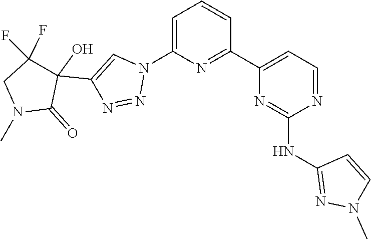 [SMILES: CN1CC(F)(F)C(O)(c2cn(-c3cccc(-c4ccnc(Nc5ccn(C)n5)n4)n3)nn2)C1=O]

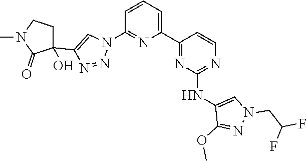 [SMILES: COc1nn(CC(F)F)cc1Nc1nccc(-c2cccc(-n3cc(C4(O)CCN(C)C4=O)nn3)n2)n1]

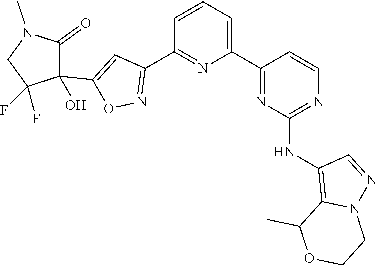 [SMILES: CC1OCCn2ncc(Nc3nccc(-c4cccc(-c5cc(C6(O)C(=O)N(C)CC6(F)F)on5)n4)n3)c21]

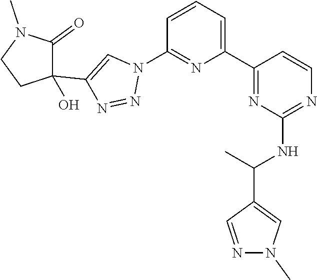 [SMILES: CC(Nc1nccc(-c2cccc(-n3cc(C4(O)CCN(C)C4=O)nn3)n2)n1)c1cnn(C)c1]

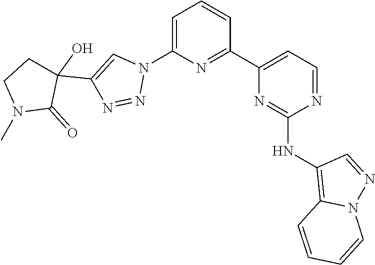 [SMILES: CN1CCC(O)(c2cn(-c3cccc(-c4ccnc(Nc5cnn6ccccc56)n4)n3)nn2)C1=O]

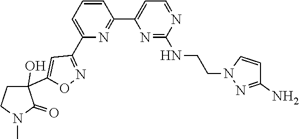 [SMILES: CN1CCC(O)(c2cc(-c3cccc(-c4ccnc(NCCn5ccc(N)n5)n4)n3)no2)C1=O]

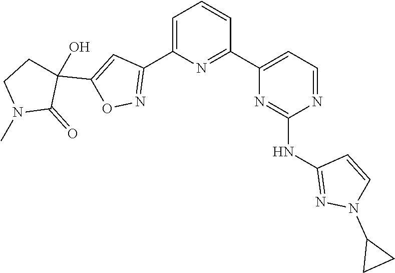 [SMILES: CN1CCC(O)(c2cc(-c3cccc(-c4ccnc(Nc5ccn(C6CC6)n5)n4)n3)no2)C1=O]

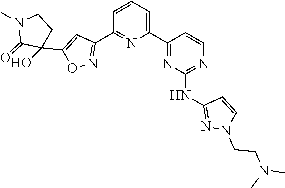

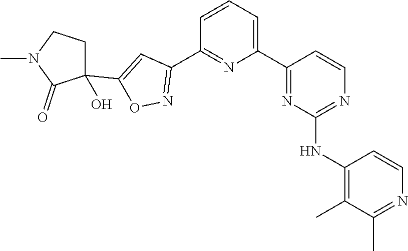 [SMILES: Cc1nccc(Nc2nccc(-c3cccc(-c4cc(C5(O)CCN(C)C5=O)on4)n3)n2)c1C]

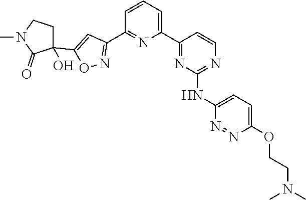

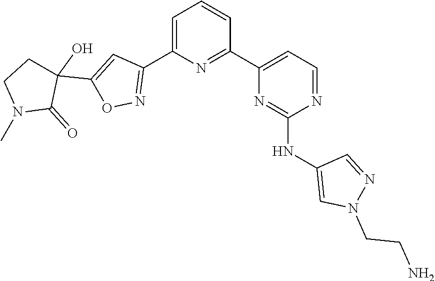 [SMILES: CN1CCC(O)(c2cc(-c3cccc(-c4ccnc(Nc5cnn(CCN)c5)n4)n3)no2)C1=O]

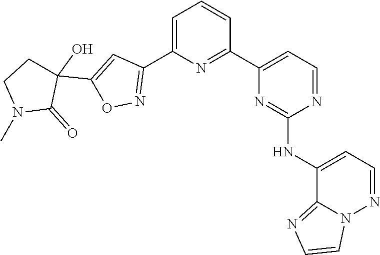 [SMILES: CN1CCC(O)(c2cc(-c3cccc(-c4ccnc(Nc5ccnn6ccnc56)n4)n3)no2)C1=O]

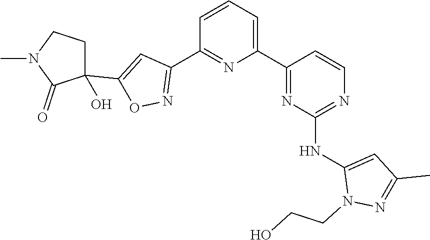

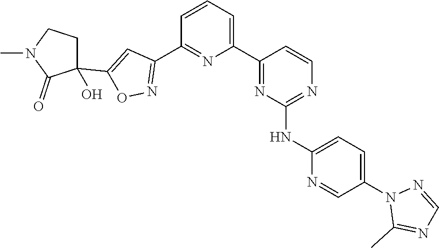 [SMILES: Cc1ncnn1-c1ccc(Nc2nccc(-c3cccc(-c4cc(C5(O)CCN(C)C5=O)on4)n3)n2)nc1]

TABLE 1G

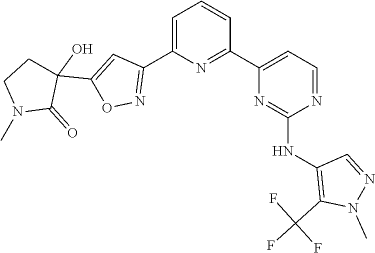 [SMILES: CN1CCC(O)(c2cc(-c3cccc(-c4ccnc(Nc5cnn(C)c5C(F)(F)F)n4)n3)no2)C1=O]

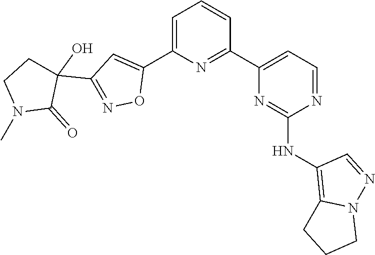 [SMILES: CN1CCC(O)(c2cc(-c3cccc(-c4ccnc(Nc5cnn6c5CCC6)n4)n3)on2)C1=O]

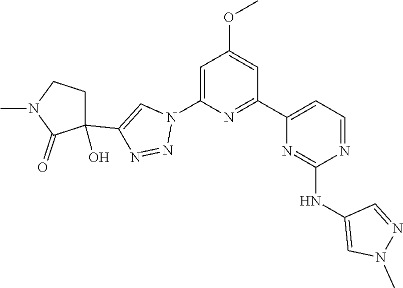 [SMILES: COc1cc(-c2ccnc(Nc3cnn(C)c3)n2)nc(-n2cc(C3(O)CCN(C)C3=O)nn2)c1]

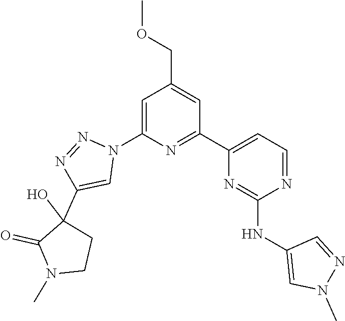 [SMILES: COCc1cc(-c2ccnc(Nc3cnn(C)c3)n2)nc(-n2cc(C3(O)CCN(C)C3=O)nn2)c1]

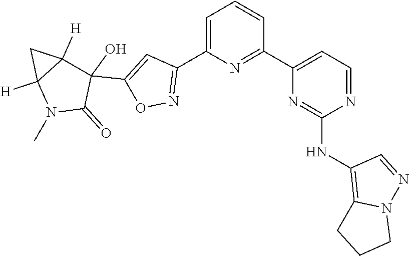 [SMILES: CN1C(=O)C(O)(c2cc(-c3cccc(-c4ccnc(Nc5cnn6c5CCC6)n4)n3)no2)C2CC21]

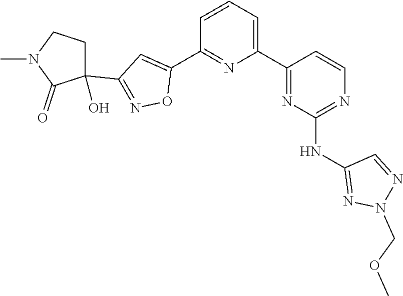 [SMILES: COCn1ncc(Nc2nccc(-c3cccc(-c4cc(C5(O)CCN(C)C5=O)no4)n3)n2)n1]

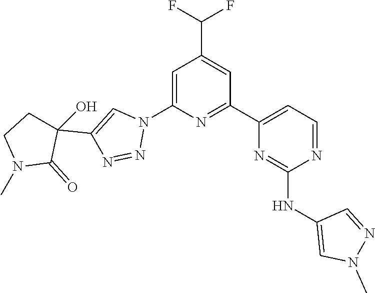 [SMILES: CN1CCC(O)(c2cn(-c3cc(C(F)F)cc(-c4ccnc(Nc5cnn(C)c5)n4)n3)nn2)C1=O]

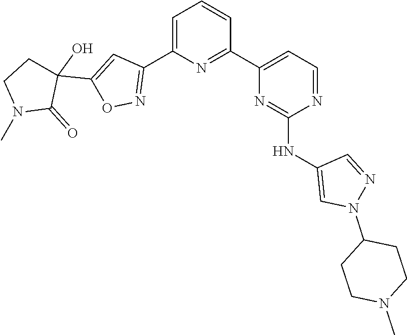 [SMILES: CN1CCC(n2cc(Nc3nccc(-c4cccc(-c5cc(C6(O)CCN(C)C6=O)on5)n4)n3)cn2)CC1]

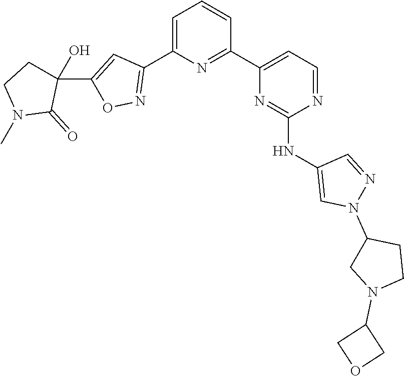 [SMILES: CN1CCC(O)(c2cc(-c3cccc(-c4ccnc(Nc5cnn(C6CCN(C7COC7)C6)c5)n4)n3)no2)C1=O]

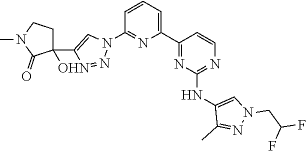 [SMILES: Cc1nn(CC(F)F)cc1Nc1nccc(-c2cccc(-n3cc(C4(O)CCN(C)C4=O)nn3)n2)n1]

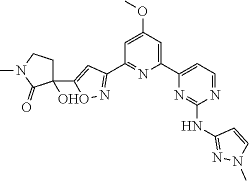 [SMILES: COc1cc(-c2cc(C3(O)CCN(C)C3=O)on2)nc(-c2ccnc(Nc3ccn(C)n3)n2)c1]

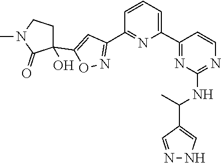 [SMILES: CC(Nc1nccc(-c2cccc(-c3cc(C4(O)CCN(C)C4=O)on3)n2)n1)c1cn[nH]c1]

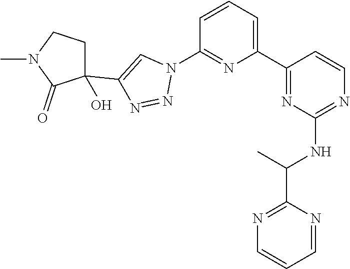 [SMILES: CC(Nc1nccc(-c2cccc(-n3cc(C4(O)CCN(C)C4=O)nn3)n2)n1)c1ncccn1]

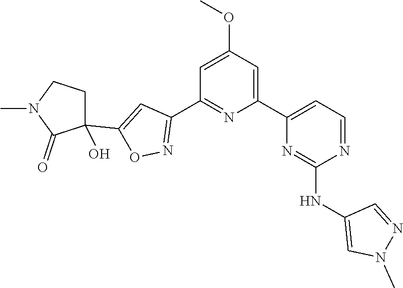 [SMILES: COc1cc(-c2cc(C3(O)CCN(C)C3=O)on2)nc(-c2ccnc(Nc3cnn(C)c3)n2)c1]

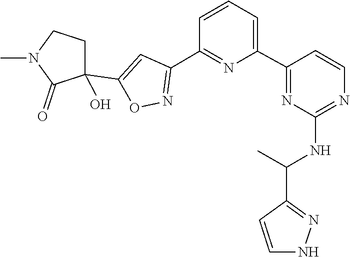 [SMILES: CC(Nc1nccc(-c2cccc(-c3cc(C4(O)CCN(C)C4=O)on3)n2)n1)c1cc[nH]n1]

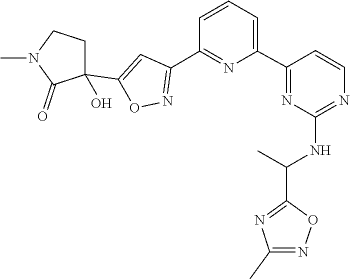 [SMILES: Cc1noc(C(C)Nc2nccc(-c3cccc(-c4cc(C5(O)CCN(C)C5=O)on4)n3)n2)n1]

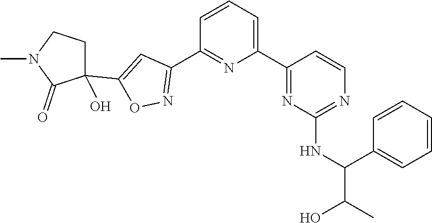 [SMILES: CC(O)C(Nc1nccc(-c2cccc(-c3cc(C4(O)CCN(C)C4=O)on3)n2)n1)c1ccccc1]

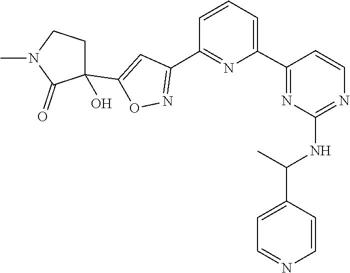 [SMILES: CC(Nc1nccc(-c2cccc(-c3cc(C4(O)CCN(C)C4=O)on3)n2)n1)c1ccncc1]

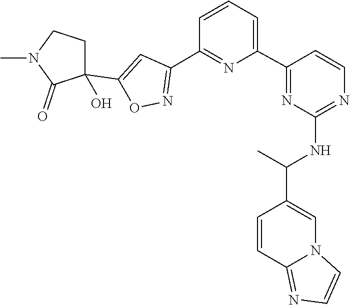 [SMILES: CC(Nc1nccc(-c2cccc(-c3cc(C4(O)CCN(C)C4=O)on3)n2)n1)c1ccc2nccn2c1]

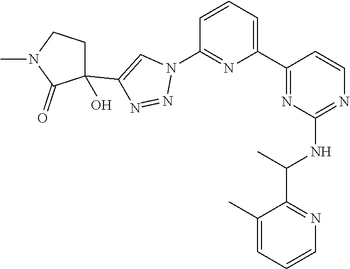 [SMILES: Cc1cccnc1C(C)Nc1nccc(-c2cccc(-n3cc(C4(O)CCN(C)C4=O)nn3)n2)n1]

TABLE 1H

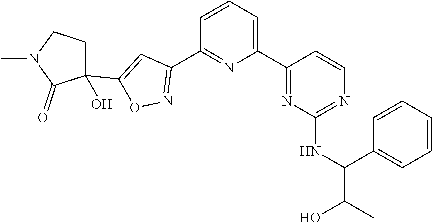 [SMILES: CC(O)C(Nc1nccc(-c2cccc(-c3cc(C4(O)CCN(C)C4=O)on3)n2)n1)c1ccccc1]

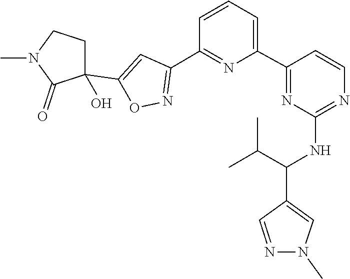 [SMILES: CC(C)C(Nc1nccc(-c2cccc(-c3cc(C4(O)CCN(C)C4=O)on3)n2)n1)c1cnn(C)c1]

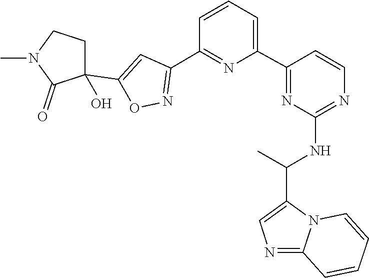 [SMILES: CC(Nc1nccc(-c2cccc(-c3cc(C4(O)CCN(C)C4=O)on3)n2)n1)c1cnc2ccccn12]

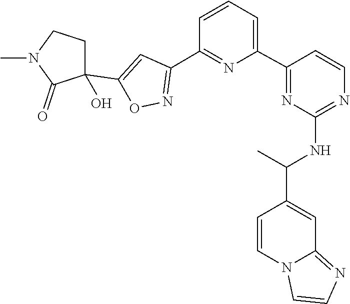 [SMILES: CC(Nc1nccc(-c2cccc(-c3cc(C4(O)CCN(C)C4=O)on3)n2)n1)c1ccn2ccnc2c1]

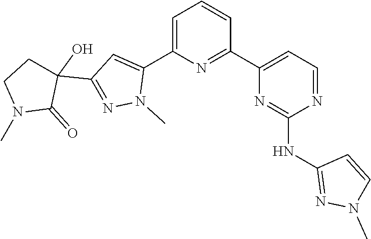 [SMILES: CN1CCC(O)(c2cc(-c3cccc(-c4ccnc(Nc5ccn(C)n5)n4)n3)n(C)n2)C1=O]

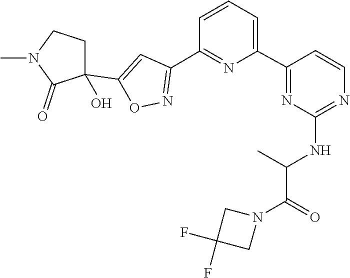 [SMILES: CC(Nc1nccc(-c2cccc(-c3cc(C4(O)CCN(C)C4=O)on3)n2)n1)C(=O)N1CC(F)(F)C1]

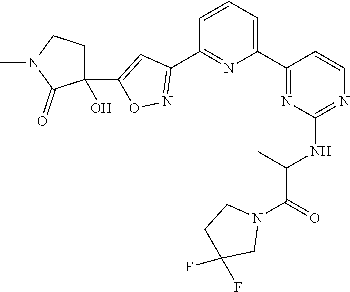 [SMILES: CC(Nc1nccc(-c2cccc(-c3cc(C4(O)CCN(C)C4=O)on3)n2)n1)C(=O)N1CCC(F)(F)C1]

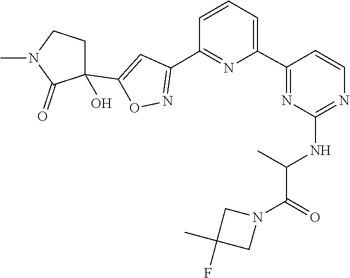 [SMILES: CC(Nc1nccc(-c2cccc(-c3cc(C4(O)CCN(C)C4=O)on3)n2)n1)C(=O)N1CC(C)(F)C1]

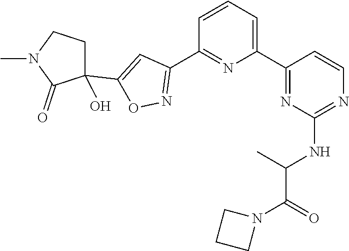 [SMILES: CC(Nc1nccc(-c2cccc(-c3cc(C4(O)CCN(C)C4=O)on3)n2)n1)C(=O)N1CCC1]

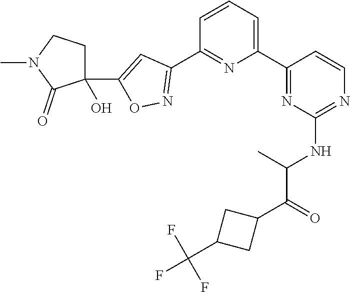 [SMILES: CC(Nc1nccc(-c2cccc(-c3cc(C4(O)CCN(C)C4=O)on3)n2)n1)C(=O)C1CC(C(F)(F)F)C1]

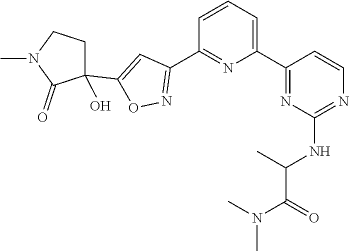 [SMILES: CC(Nc1nccc(-c2cccc(-c3cc(C4(O)CCN(C)C4=O)on3)n2)n1)C(=O)N(C)C]

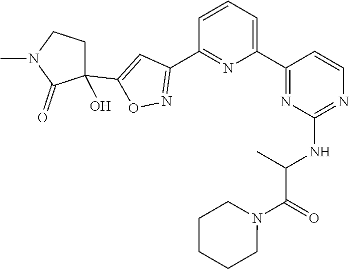 [SMILES: CC(Nc1nccc(-c2cccc(-c3cc(C4(O)CCN(C)C4=O)on3)n2)n1)C(=O)N1CCCCC1]

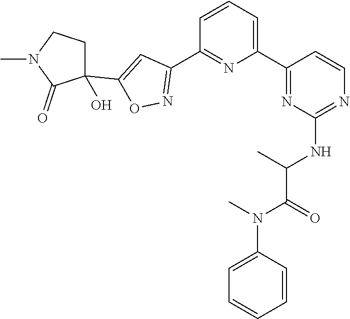 [SMILES: CC(Nc1nccc(-c2cccc(-c3cc(C4(O)CCN(C)C4=O)on3)n2)n1)C(=O)N(C)c1ccccc1]

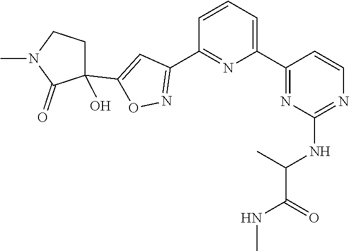 [SMILES: CNC(=O)C(C)Nc1nccc(-c2cccc(-c3cc(C4(O)CCN(C)C4=O)on3)n2)n1]

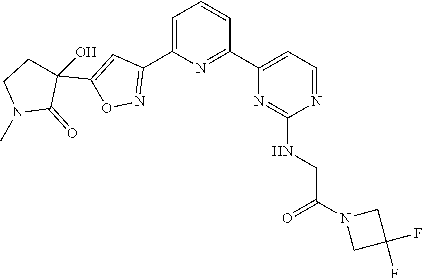 [SMILES: CN1CCC(O)(c2cc(-c3cccc(-c4ccnc(NCC(=O)N5CC(F)(F)C5)n4)n3)no2)C1=O]

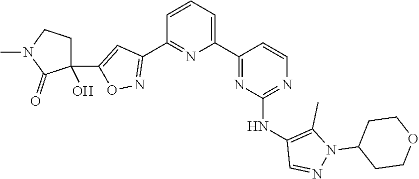 [SMILES: Cc1c(Nc2nccc(-c3cccc(-c4cc(C5(O)CCN(C)C5=O)on4)n3)n2)cnn1C1CCOCC1]

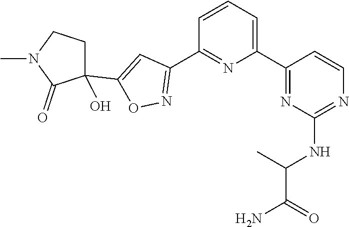 [SMILES: CC(Nc1nccc(-c2cccc(-c3cc(C4(O)CCN(C)C4=O)on3)n2)n1)C(N)=O]

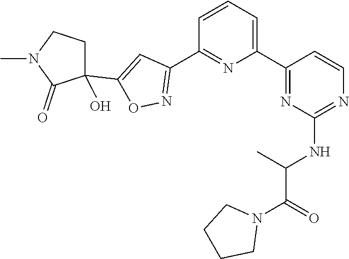 [SMILES: CC(Nc1nccc(-c2cccc(-c3cc(C4(O)CCN(C)C4=O)on3)n2)n1)C(=O)N1CCCC1]

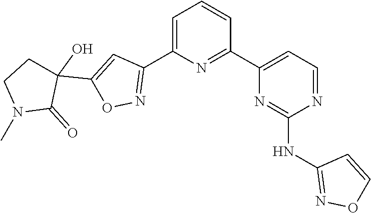 [SMILES: CN1CCC(O)(c2cc(-c3cccc(-c4ccnc(Nc5ccon5)n4)n3)no2)C1=O]

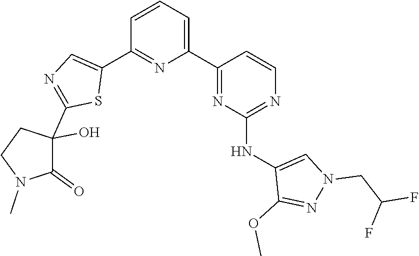 [SMILES: COc1nn(CC(F)F)cc1Nc1nccc(-c2cccc(-c3cnc(C4(O)CCN(C)C4=O)s3)n2)n1]

TABLE 1I

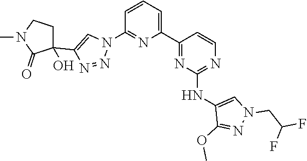 [SMILES: COc1nn(CC(F)F)cc1Nc1nccc(-c2cccc(-n3cc(C4(O)CCN(C)C4=O)nn3)n2)n1]

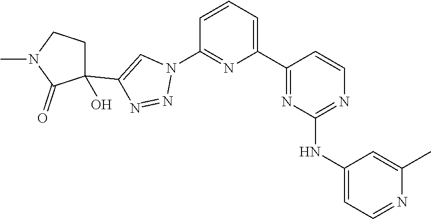

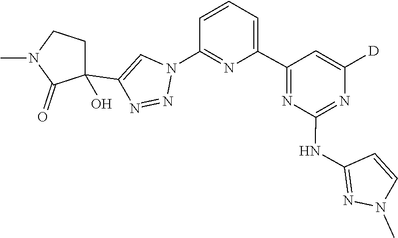 [SMILES: [2H]c1cc(-c2cccc(-n3cc(C4(O)CCN(C)C4=O)nn3)n2)nc(Nc2ccn(C)n2)n1]

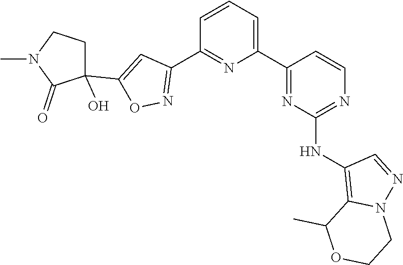 [SMILES: CC1OCCn2ncc(Nc3nccc(-c4cccc(-c5cc(C6(O)CCN(C)C6=O)on5)n4)n3)c21]

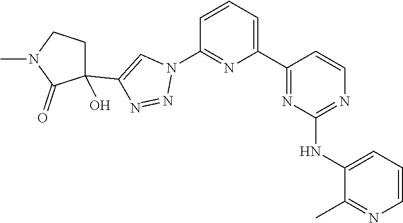 [SMILES: Cc1ncccc1Nc1nccc(-c2cccc(-n3cc(C4(O)CCN(C)C4=O)nn3)n2)n1]

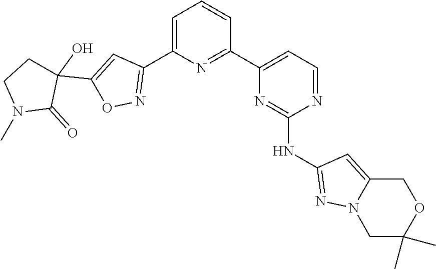

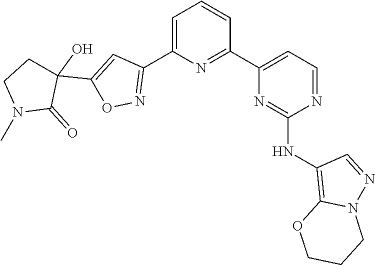 [SMILES: CN1CCC(O)(c2cc(-c3cccc(-c4ccnc(Nc5cnn6c5OCCC6)n4)n3)no2)C1=O]

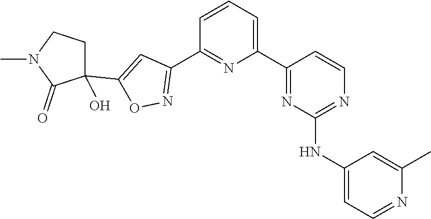

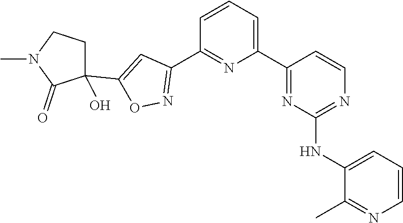 [SMILES: Cc1ncccc1Nc1nccc(-c2cccc(-c3cc(C4(O)CCN(C)C4=O)on3)n2)n1]

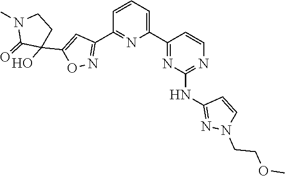

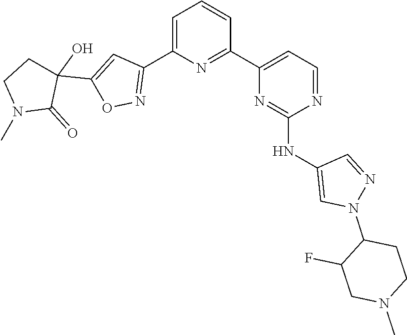 [SMILES: CN1CCC(n2cc(Nc3nccc(-c4cccc(-c5cc(C6(O)CCN(C)C6=O)on5)n4)n3)cn2)C(F)C1]

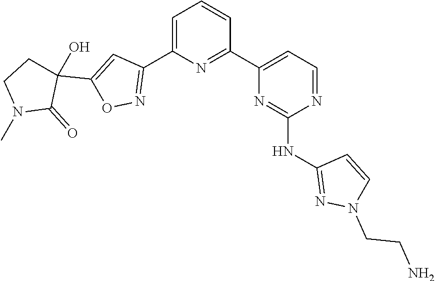 [SMILES: CN1CCC(O)(c2cc(-c3cccc(-c4ccnc(Nc5ccn(CCN)n5)n4)n3)no2)C1=O]

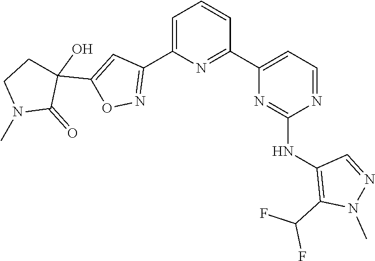 [SMILES: CN1CCC(O)(c2cc(-c3cccc(-c4ccnc(Nc5cnn(C)c5C(F)F)n4)n3)no2)C1=O]

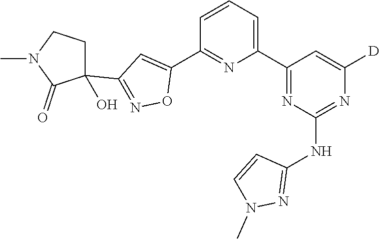 [SMILES: [2H]c1cc(-c2cccc(-c3cc(C4(O)CCN(C)C4=O)no3)n2)nc(Nc2ccn(C)n2)n1]

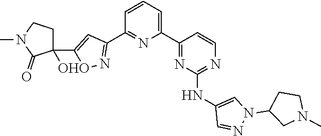

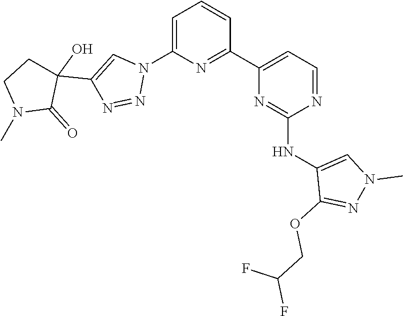

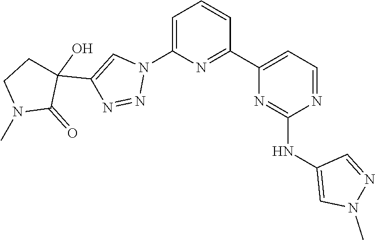 [SMILES: CN1CCC(O)(c2cn(-c3cccc(-c4ccnc(Nc5cnn(C)c5)n4)n3)nn2)C1=O]

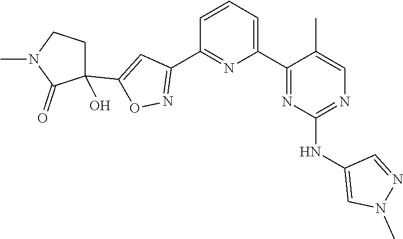 [SMILES: Cc1cnc(Nc2cnn(C)c2)nc1-c1cccc(-c2cc(C3(O)CCN(C)C3=O)on2)n1]

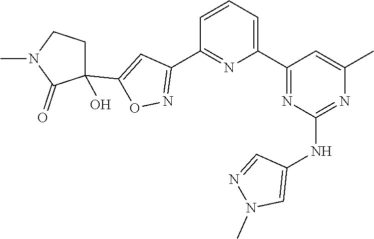

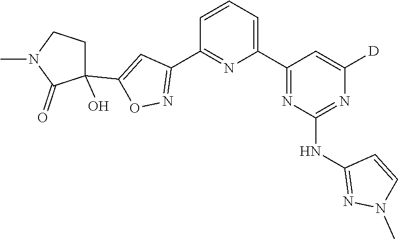 [SMILES: [2H]c1cc(-c2cccc(-c3cc(C4(O)CCN(C)C4=O)on3)n2)nc(Nc2ccn(C)n2)n1]

TABLE 1J

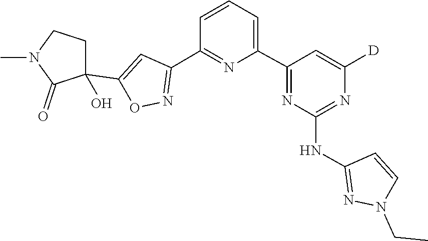 [SMILES: [2H]c1cc(-c2cccc(-c3cc(C4(O)CCN(C)C4=O)on3)n2)nc(Nc2ccn(CC)n2)n1]

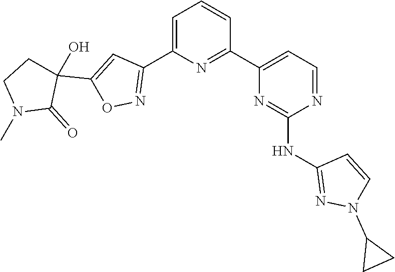 [SMILES: CN1CCC(O)(c2cc(-c3cccc(-c4ccnc(Nc5ccn(C6CC6)n5)n4)n3)no2)C1=O]

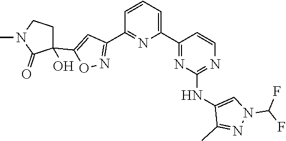 [SMILES: Cc1nn(C(F)F)cc1Nc1nccc(-c2cccc(-c3cc(C4(O)CCN(C)C4=O)on3)n2)n1]

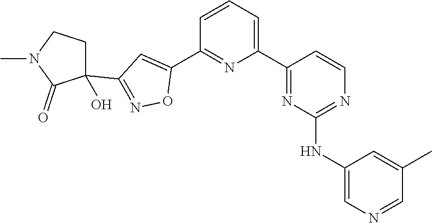

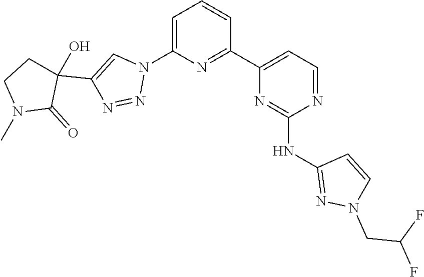 [SMILES: CN1CCC(O)(c2cn(-c3cccc(-c4ccnc(Nc5ccn(CC(F)F)n5)n4)n3)nn2)C1=O]

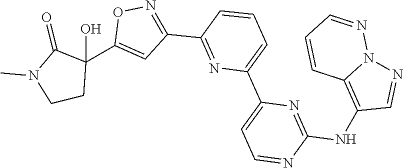 [SMILES: CN1CCC(O)(c2cc(-c3cccc(-c4ccnc(Nc5cnn6ncccc56)n4)n3)no2)C1=O]

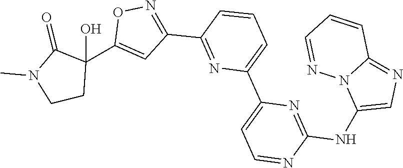 [SMILES: CN1CCC(O)(c2cc(-c3cccc(-c4ccnc(Nc5cnc6cccnn56)n4)n3)no2)C1=O]

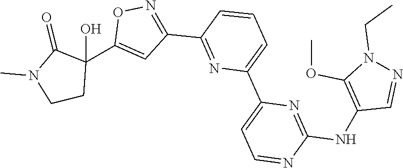 [SMILES: CCn1ncc(Nc2nccc(-c3cccc(-c4cc(C5(O)CCN(C)C5=O)on4)n3)n2)c1OC]

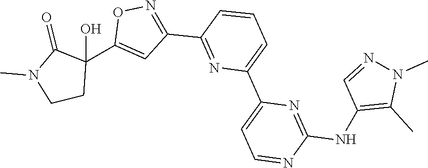

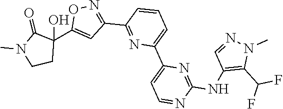 [SMILES: CN1CCC(O)(c2cc(-c3cccc(-c4ccnc(Nc5cnn(C)c5C(F)F)n4)n3)no2)C1=O]

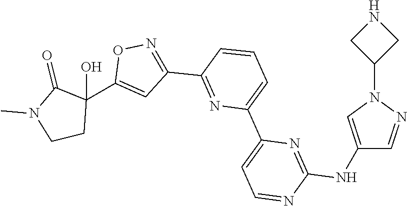 [SMILES: CN1CCC(O)(c2cc(-c3cccc(-c4ccnc(Nc5cnn(C6CNC6)c5)n4)n3)no2)C1=O]

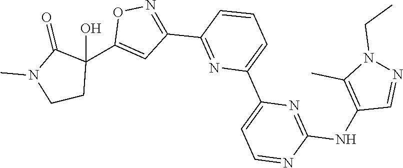 [SMILES: CCn1ncc(Nc2nccc(-c3cccc(-c4cc(C5(O)CCN(C)C5=O)on4)n3)n2)c1C]

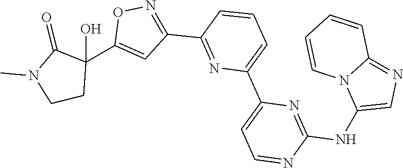 [SMILES: CN1CCC(O)(c2cc(-c3cccc(-c4ccnc(Nc5cnc6ccccn56)n4)n3)no2)C1=O]

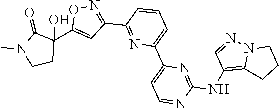 [SMILES: CN1CCC(O)(c2cc(-c3cccc(-c4ccnc(Nc5cnn6c5CCC6)n4)n3)no2)C1=O]

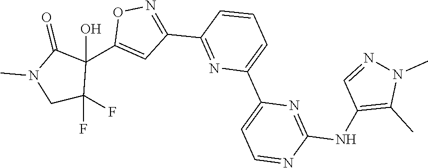

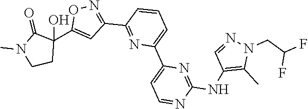 [SMILES: Cc1c(Nc2nccc(-c3cccc(-c4cc(C5(O)CCN(C)C5=O)on4)n3)n2)cnn1CC(F)F]

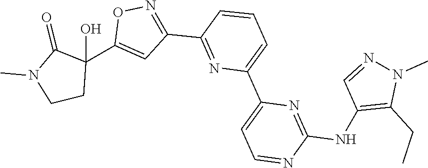

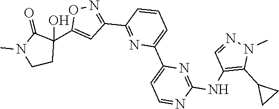 [SMILES: CN1CCC(O)(c2cc(-c3cccc(-c4ccnc(Nc5cnn(C)c5C5CC5)n4)n3)no2)C1=O]

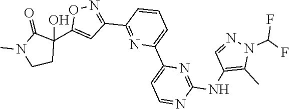 [SMILES: Cc1c(Nc2nccc(-c3cccc(-c4cc(C5(O)CCN(C)C5=O)on4)n3)n2)cnn1C(F)F]

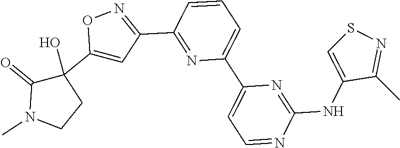

TABLE 1K

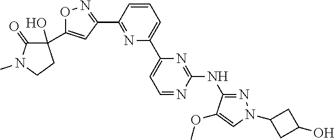 [SMILES: COc1cn(C2CC(O)C2)nc1Nc1nccc(-c2cccc(-c3cc(C4(O)CCN(C)C4=O)on3)n2)n1]

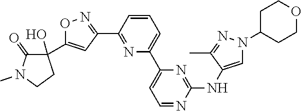 [SMILES: Cc1nn(C2CCOCC2)cc1Nc1nccc(-c2cccc(-c3cc(C4(O)CCN(C)C4=O)on3)n2)n1]

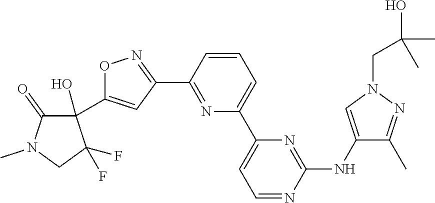 [SMILES: Cc1nn(CC(C)(C)O)cc1Nc1nccc(-c2cccc(-c3cc(C4(O)C(=O)N(C)CC4(F)F)on3)n2)n1]

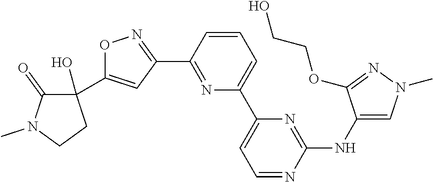

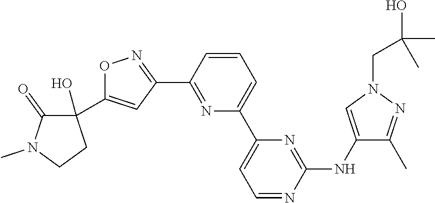 [SMILES: Cc1nn(CC(C)(C)O)cc1Nc1nccc(-c2cccc(-c3cc(C4(O)CCN(C)C4=O)on3)n2)n1]

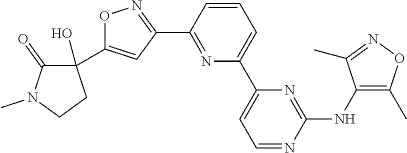

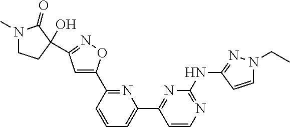 [SMILES: CCn1ccc(Nc2nccc(-c3cccc(-c4cc(C5(O)CCN(C)C5=O)no4)n3)n2)n1]

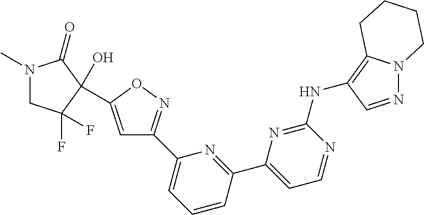 [SMILES: CN1CC(F)(F)C(O)(c2cc(-c3cccc(-c4ccnc(Nc5cnn6c5CCCC6)n4)n3)no2)C1=O]

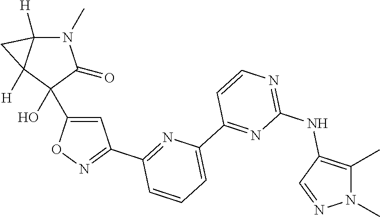 [SMILES: Cc1c(Nc2nccc(-c3cccc(-c4cc(C5(O)C(=O)N(C)C6CC65)on4)n3)n2)cnn1C]

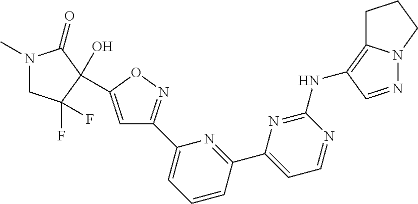 [SMILES: CN1CC(F)(F)C(O)(c2cc(-c3cccc(-c4ccnc(Nc5cnn6c5CCC6)n4)n3)no2)C1=O]

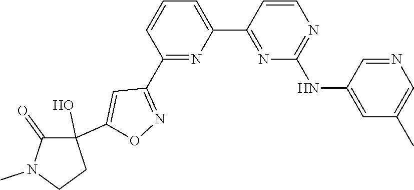

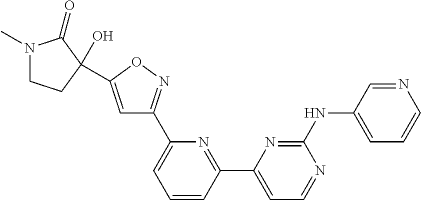 [SMILES: CN1CCC(O)(c2cc(-c3cccc(-c4ccnc(Nc5cccnc5)n4)n3)no2)C1=O]

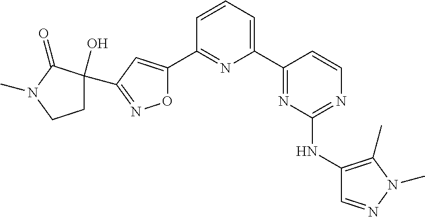

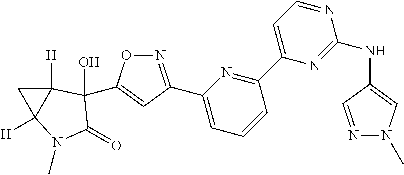 [SMILES: CN1C(=O)C(O)(c2cc(-c3cccc(-c4ccnc(Nc5cnn(C)c5)n4)n3)no2)C2CC21]

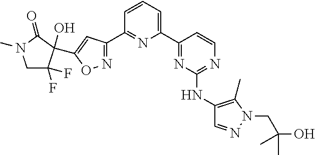 [SMILES: Cc1c(Nc2nccc(-c3cccc(-c4cc(C5(O)C(=O)N(C)CC5(F)F)on4)n3)n2)cnn1CC(C)(C)O]

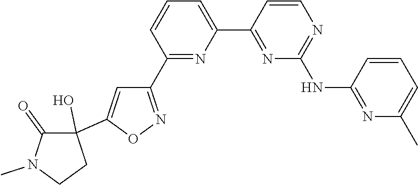

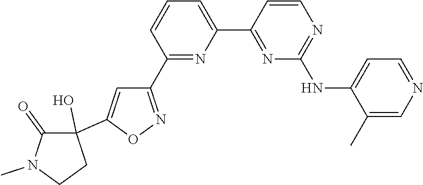 [SMILES: Cc1cnccc1Nc1nccc(-c2cccc(-c3cc(C4(O)CCN(C)C4=O)on3)n2)n1]

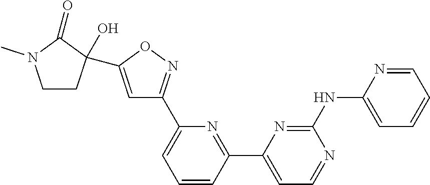 [SMILES: CN1CCC(O)(c2cc(-c3cccc(-c4ccnc(Nc5ccccn5)n4)n3)no2)C1=O]

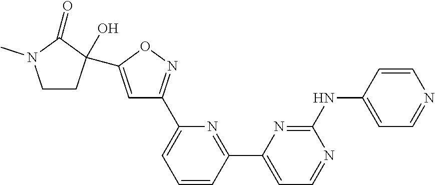 [SMILES: CN1CCC(O)(c2cc(-c3cccc(-c4ccnc(Nc5ccncc5)n4)n3)no2)C1=O]

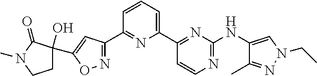 [SMILES: CCn1cc(Nc2nccc(-c3cccc(-c4cc(C5(O)CCN(C)C5=O)on4)n3)n2)c(C)n1]

TABLE 1L

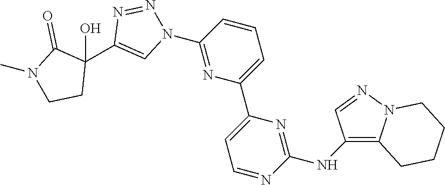 [SMILES: CN1CCC(O)(c2cn(-c3cccc(-c4ccnc(Nc5cnn6c5CCCC6)n4)n3)nn2)C1=O]

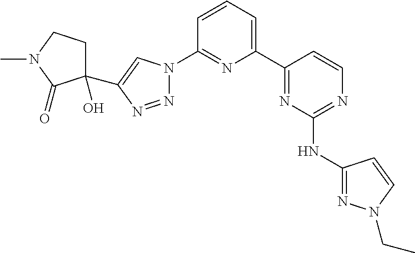 [SMILES: CCn1ccc(Nc2nccc(-c3cccc(-n4cc(C5(O)CCN(C)C5=O)nn4)n3)n2)n1]

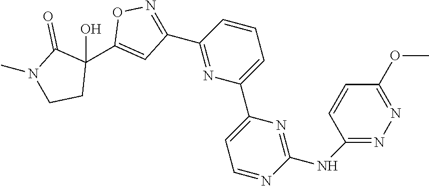

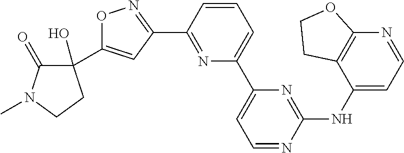 [SMILES: CN1CCC(O)(c2cc(-c3cccc(-c4ccnc(Nc5ccnc6c5CCO6)n4)n3)no2)C1=O]

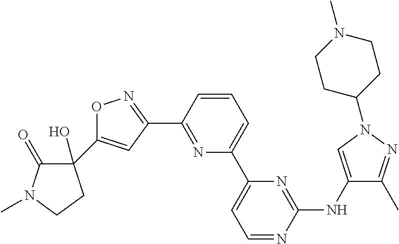

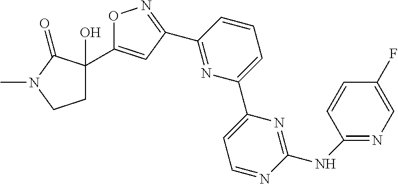 [SMILES: CN1CCC(O)(c2cc(-c3cccc(-c4ccnc(Nc5ccc(F)cn5)n4)n3)no2)C1=O]

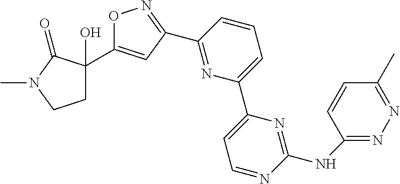

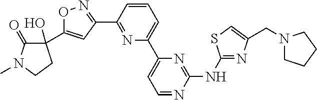 [SMILES: CN1CCC(O)(c2cc(-c3cccc(-c4ccnc(Nc5nc(CN6CCCC6)cs5)n4)n3)no2)C1=O]

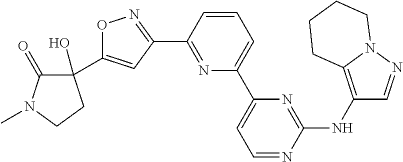 [SMILES: CN1CCC(O)(c2cc(-c3cccc(-c4ccnc(Nc5cnn6c5CCCC6)n4)n3)no2)C1=O]

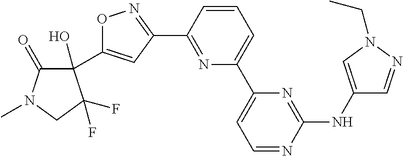 [SMILES: CCn1cc(Nc2nccc(-c3cccc(-c4cc(C5(O)C(=O)N(C)CC5(F)F)on4)n3)n2)cn1]

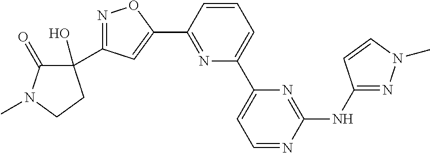

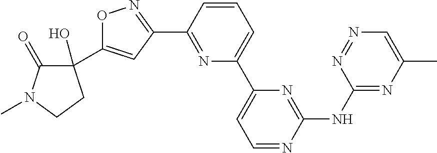

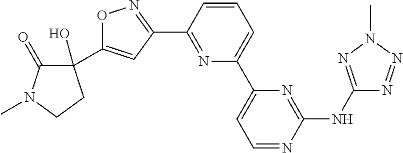 [SMILES: CN1CCC(O)(c2cc(-c3cccc(-c4ccnc(Nc5nnn(C)n5)n4)n3)no2)C1=O]

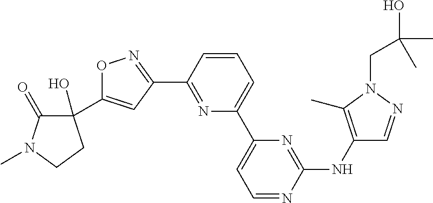 [SMILES: Cc1c(Nc2nccc(-c3cccc(-c4cc(C5(O)CCN(C)C5=O)on4)n3)n2)cnn1CC(C)(C)O]

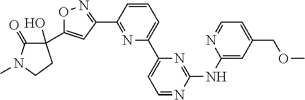

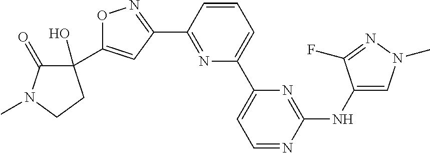

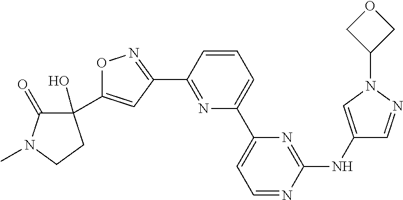 [SMILES: CN1CCC(O)(c2cc(-c3cccc(-c4ccnc(Nc5cnn(C6COC6)c5)n4)n3)no2)C1=O]

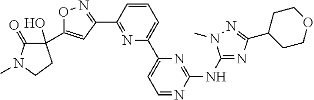 [SMILES: CN1CCC(O)(c2cc(-c3cccc(-c4ccnc(Nc5nc(C6CCOCC6)nn5C)n4)n3)no2)C1=O]

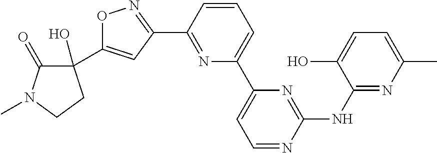

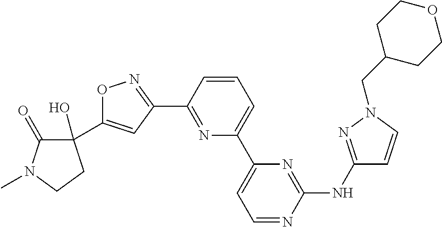 [SMILES: CN1CCC(O)(c2cc(-c3cccc(-c4ccnc(Nc5ccn(CC6CCOCC6)n5)n4)n3)no2)C1=O]

TABLE 1M

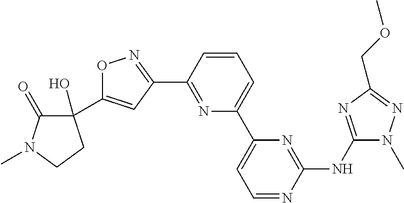

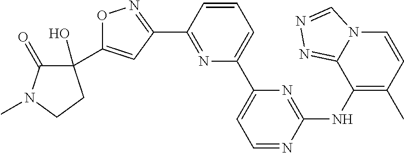

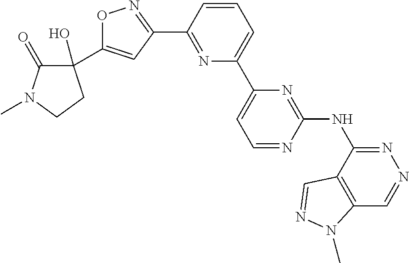 [SMILES: CN1CCC(O)(c2cc(-c3cccc(-c4ccnc(Nc5nncc6c5cnn6C)n4)n3)no2)C1=O]

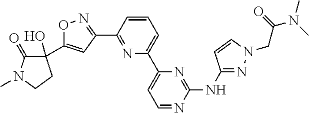

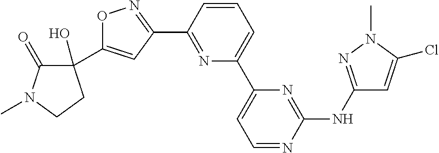 [SMILES: CN1CCC(O)(c2cc(-c3cccc(-c4ccnc(Nc5cc(Cl)n(C)n5)n4)n3)no2)C1=O]

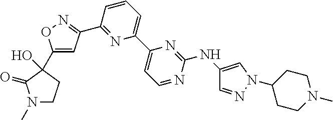 [SMILES: CN1CCC(n2cc(Nc3nccc(-c4cccc(-c5cc(C6(O)CCN(C)C6=O)on5)n4)n3)cn2)CC1]

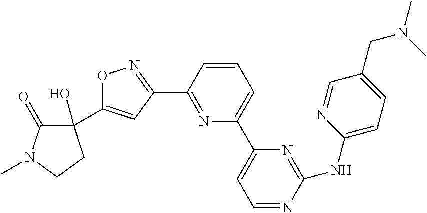

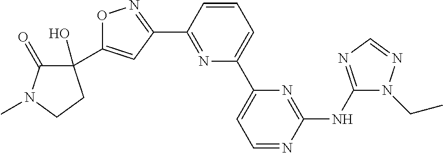 [SMILES: CCn1ncnc1Nc1nccc(-c2cccc(-c3cc(C4(O)CCN(C)C4=O)on3)n2)n1]

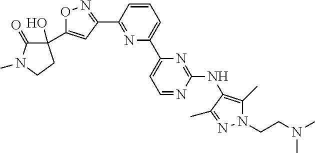

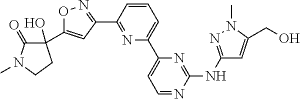 [SMILES: CN1CCC(O)(c2cc(-c3cccc(-c4ccnc(Nc5cc(CO)n(C)n5)n4)n3)no2)C1=O]

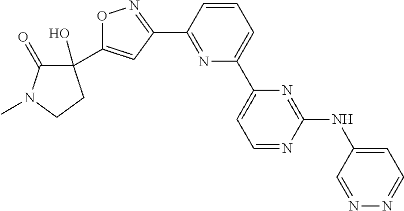 [SMILES: CN1CCC(O)(c2cc(-c3cccc(-c4ccnc(Nc5ccnnc5)n4)n3)no2)C1=O]

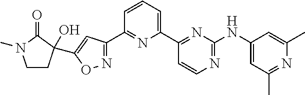

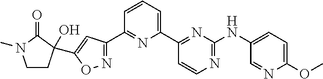

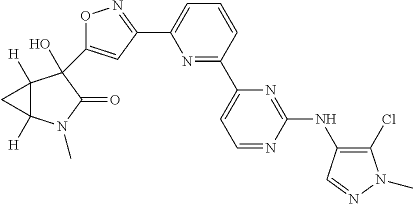 [SMILES: CN1C(=O)C(O)(c2cc(-c3cccc(-c4ccnc(Nc5cnn(C)c5Cl)n4)n3)no2)C2CC21]

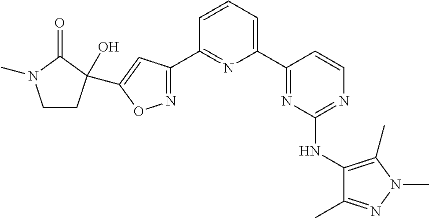

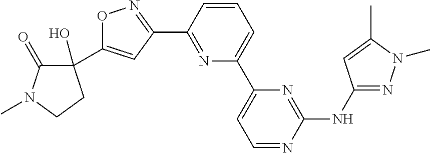

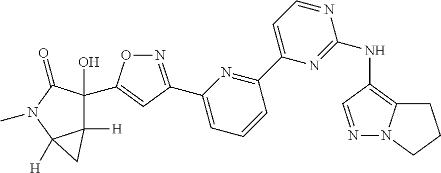 [SMILES: CN1C(=O)C(O)(c2cc(-c3cccc(-c4ccnc(Nc5cnn6c5CCC6)n4)n3)no2)C2CC21]

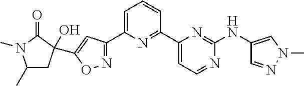

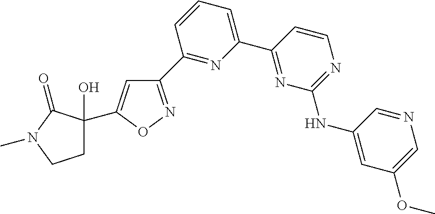

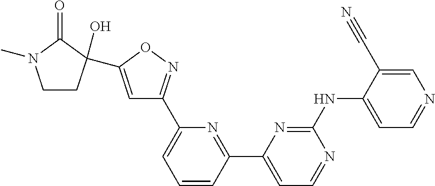 [SMILES: CN1CCC(O)(c2cc(-c3cccc(-c4ccnc(Nc5ccncc5C#N)n4)n3)no2)C1=O]

TABLE 1N

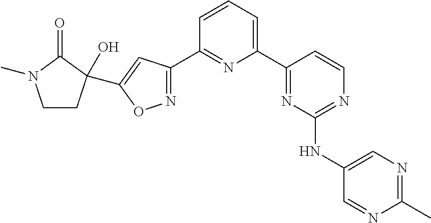

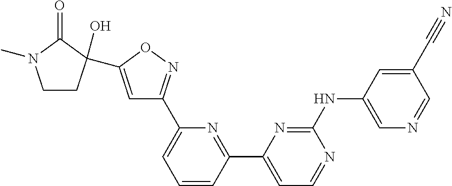 [SMILES: CN1CCC(O)(c2cc(-c3cccc(-c4ccnc(Nc5cncc(C#N)c5)n4)n3)no2)C1=O]

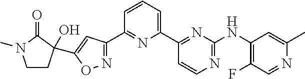

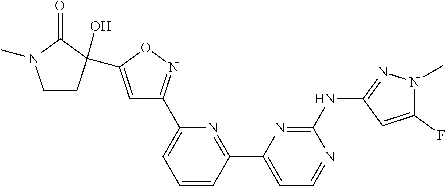

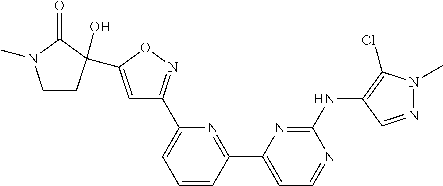

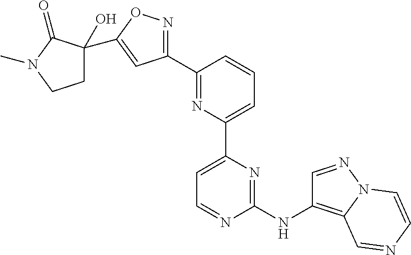 [SMILES: CN1CCC(O)(c2cc(-c3cccc(-c4ccnc(Nc5cnn6ccncc56)n4)n3)no2)C1=O]

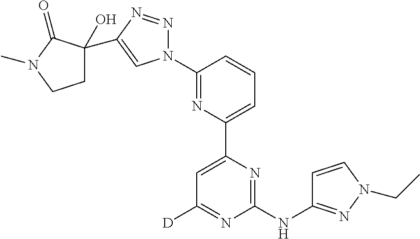 [SMILES: [2H]c1cc(-c2cccc(-n3cc(C4(O)CCN(C)C4=O)nn3)n2)nc(Nc2ccn(CC)n2)n1]

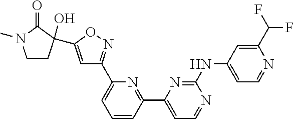 [SMILES: CN1CCC(O)(c2cc(-c3cccc(-c4ccnc(Nc5ccnc(C(F)F)c5)n4)n3)no2)C1=O]

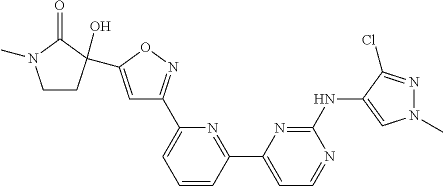

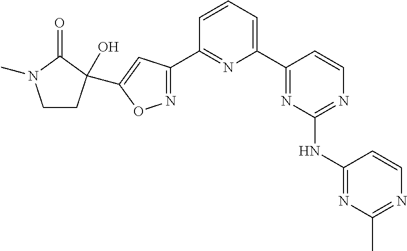 [SMILES: Cc1nccc(Nc2nccc(-c3cccc(-c4cc(C5(O)CCN(C)C5=O)on4)n3)n2)n1]

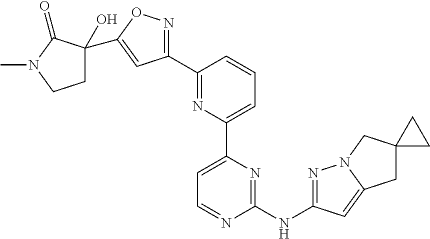 [SMILES: CN1CCC(O)(c2cc(-c3cccc(-c4ccnc(Nc5cc6n(n5)CC5(CC5)C6)n4)n3)no2)C1=O]

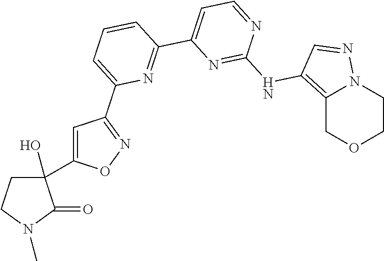 [SMILES: CN1CCC(O)(c2cc(-c3cccc(-c4ccnc(Nc5cnn6c5COCC6)n4)n3)no2)C1=O]

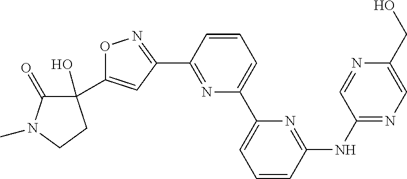 [SMILES: CN1CCC(O)(c2cc(-c3cccc(-c4cccc(Nc5cnc(CO)cn5)n4)n3)no2)C1=O]

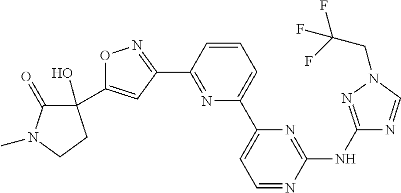 [SMILES: CN1CCC(O)(c2cc(-c3cccc(-c4ccnc(Nc5ncn(CC(F)(F)F)n5)n4)n3)no2)C1=O]

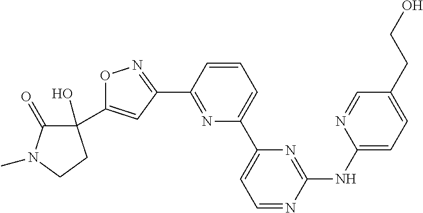 [SMILES: CN1CCC(O)(c2cc(-c3cccc(-c4ccnc(Nc5ccc(CCO)cn5)n4)n3)no2)C1=O]

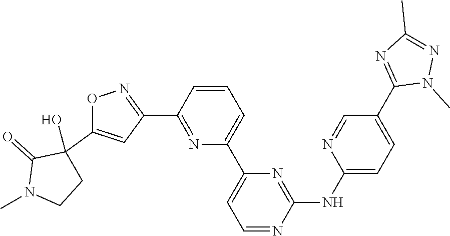

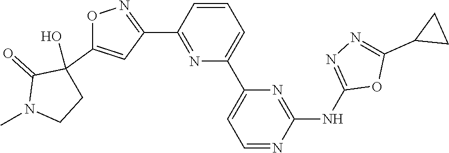 [SMILES: CN1CCC(O)(c2cc(-c3cccc(-c4ccnc(Nc5nnc(C6CC6)o5)n4)n3)no2)C1=O]

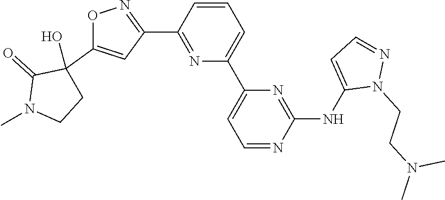

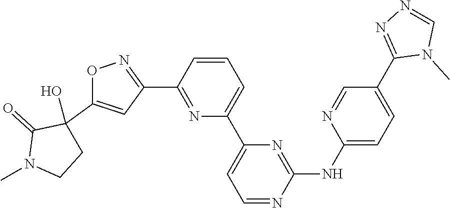

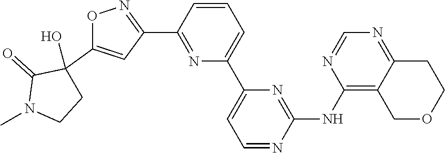 [SMILES: CN1CCC(O)(c2cc(-c3cccc(-c4ccnc(Nc5ncnc6c5COCC6)n4)n3)no2)C1=O]

TABLE 1O

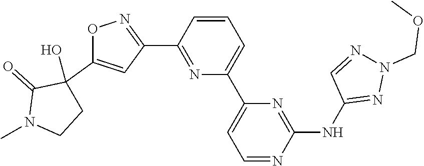 [SMILES: COCn1ncc(Nc2nccc(-c3cccc(-c4cc(C5(O)CCN(C)C5=O)on4)n3)n2)n1]

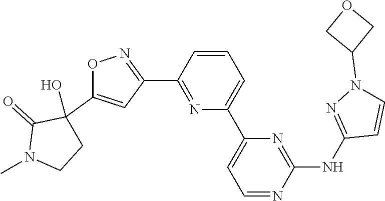 [SMILES: CN1CCC(O)(c2cc(-c3cccc(-c4ccnc(Nc5ccn(C6COC6)n5)n4)n3)no2)C1=O]

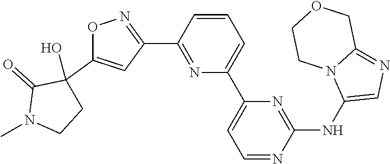 [SMILES: CN1CCC(O)(c2cc(-c3cccc(-c4ccnc(Nc5cnc6n5CCOC6)n4)n3)no2)C1=O]

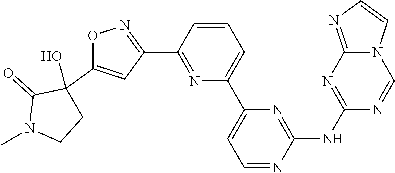 [SMILES: CN1CCC(O)(c2cc(-c3cccc(-c4ccnc(Nc5ncn6ccnc6n5)n4)n3)no2)C1=O]

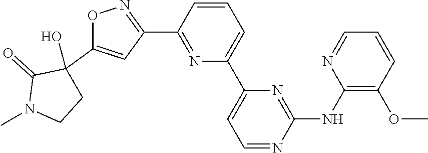

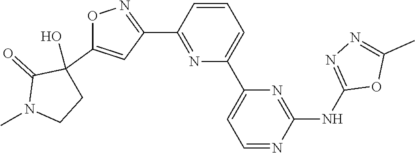

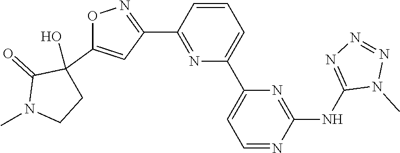

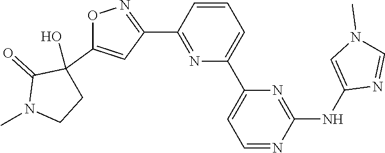 [SMILES: CN1CCC(O)(c2cc(-c3cccc(-c4ccnc(Nc5cn(C)cn5)n4)n3)no2)C1=O]

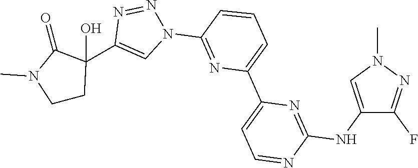

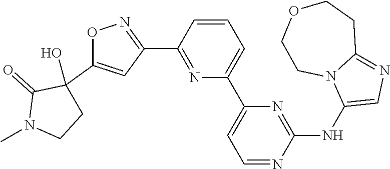 [SMILES: CN1CCC(O)(c2cc(-c3cccc(-c4ccnc(Nc5cnc6n5CCOCC6)n4)n3)no2)C1=O]

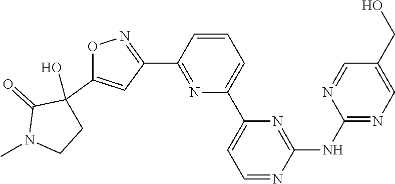 [SMILES: CN1CCC(O)(c2cc(-c3cccc(-c4ccnc(Nc5ncc(CO)cn5)n4)n3)no2)C1=O]

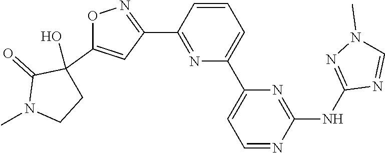 [SMILES: CN1CCC(O)(c2cc(-c3cccc(-c4ccnc(Nc5ncn(C)n5)n4)n3)no2)C1=O]

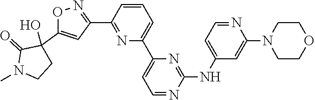 [SMILES: CN1CCC(O)(c2cc(-c3cccc(-c4ccnc(Nc5ccnc(N6CCOCC6)c5)n4)n3)no2)C1=O]

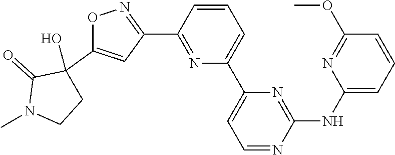 [SMILES: COc1cccc(Nc2nccc(-c3cccc(-c4cc(C5(O)CCN(C)C5=O)on4)n3)n2)n1]

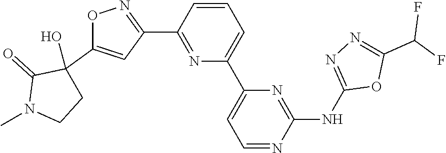 [SMILES: CN1CCC(O)(c2cc(-c3cccc(-c4ccnc(Nc5nnc(C(F)F)o5)n4)n3)no2)C1=O]

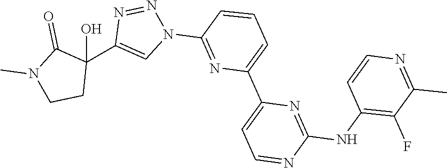

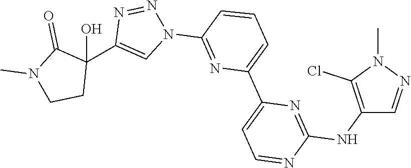 [SMILES: CN1CCC(O)(c2cn(-c3cccc(-c4ccnc(Nc5cnn(C)c5Cl)n4)n3)nn2)C1=O]

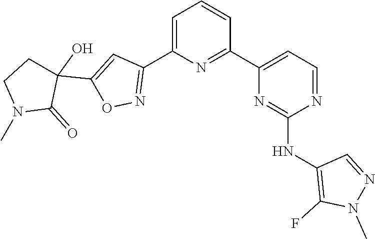 [SMILES: CN1CCC(O)(c2cc(-c3cccc(-c4ccnc(Nc5cnn(C)c5F)n4)n3)no2)C1=O]

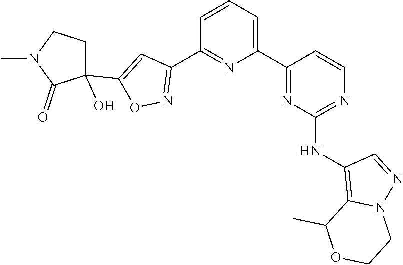 [SMILES: CC1OCCn2ncc(Nc3nccc(-c4cccc(-c5cc(C6(O)CCN(C)C6=O)on5)n4)n3)c21]

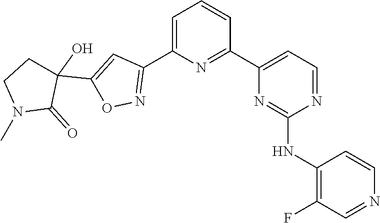 [SMILES: CN1CCC(O)(c2cc(-c3cccc(-c4ccnc(Nc5ccncc5F)n4)n3)no2)C1=O]

TABLE 1P

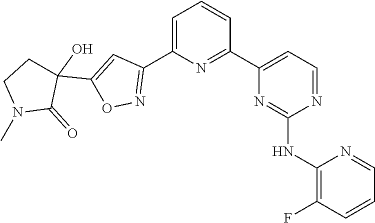 [SMILES: CN1CCC(O)(c2cc(-c3cccc(-c4ccnc(Nc5ncccc5F)n4)n3)no2)C1=O]

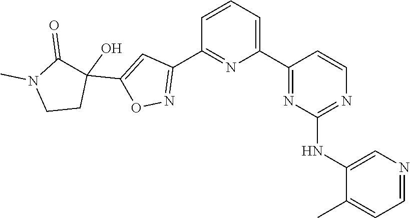 [SMILES: Cc1ccncc1Nc1nccc(-c2cccc(-c3cc(C4(O)CCN(C)C4=O)on3)n2)n1]

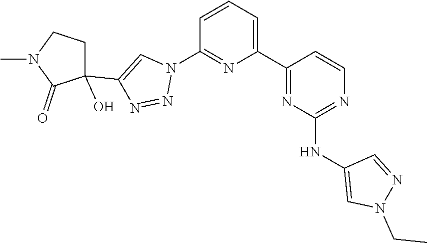 [SMILES: CCn1cc(Nc2nccc(-c3cccc(-n4cc(C5(O)CCN(C)C5=O)nn4)n3)n2)cn1]

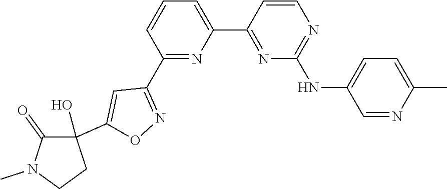

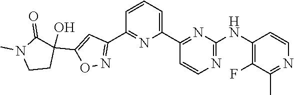 [SMILES: Cc1nccc(Nc2nccc(-c3cccc(-c4cc(C5(O)CCN(C)C5=O)on4)n3)n2)c1F]

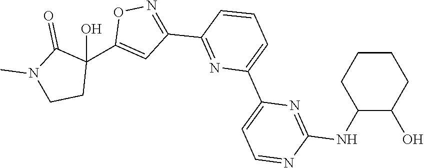 [SMILES: CN1CCC(O)(c2cc(-c3cccc(-c4ccnc(NC5CCCCC5O)n4)n3)no2)C1=O]

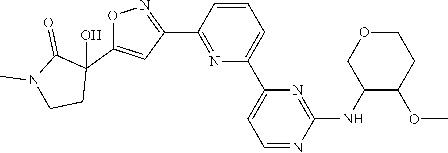

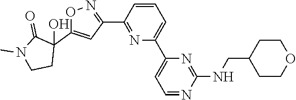 [SMILES: CN1CCC(O)(c2cc(-c3cccc(-c4ccnc(NCC5CCOCC5)n4)n3)no2)C1=O]

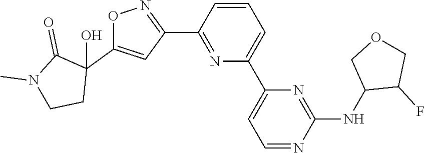 [SMILES: CN1CCC(O)(c2cc(-c3cccc(-c4ccnc(NC5COCC5F)n4)n3)no2)C1=O]

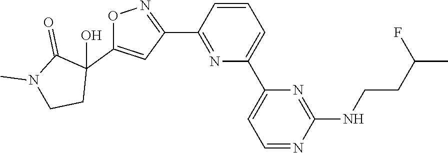 [SMILES: CC(F)CCNc1nccc(-c2cccc(-c3cc(C4(O)CCN(C)C4=O)on3)n2)n1]

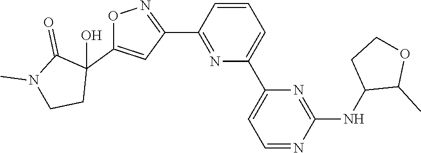

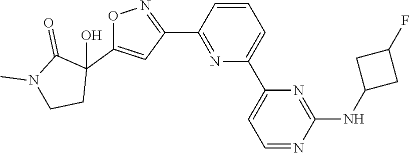 [SMILES: CN1CCC(O)(c2cc(-c3cccc(-c4ccnc(NC5CC(F)C5)n4)n3)no2)C1=O]

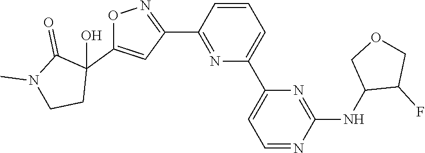 [SMILES: CN1CCC(O)(c2cc(-c3cccc(-c4ccnc(NC5COCC5F)n4)n3)no2)C1=O]

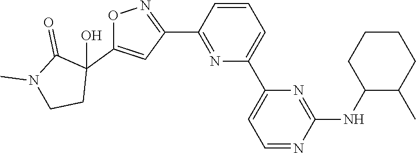

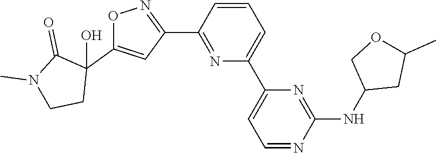

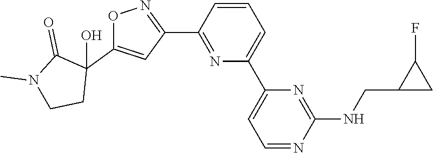 [SMILES: CN1CCC(O)(c2cc(-c3cccc(-c4ccnc(NCC5CC5F)n4)n3)no2)C1=O]

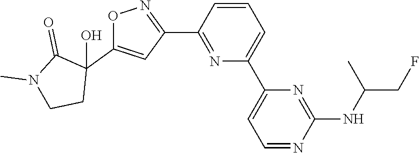 [SMILES: CC(CF)Nc1nccc(-c2cccc(-c3cc(C4(O)CCN(C)C4=O)on3)n2)n1]

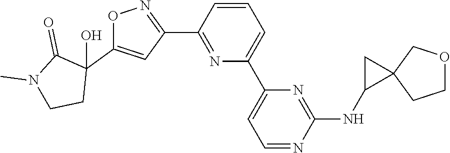 [SMILES: CN1CCC(O)(c2cc(-c3cccc(-c4ccnc(NC5CC56CCOC6)n4)n3)no2)C1=O]

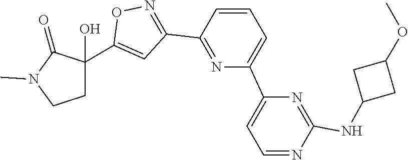 [SMILES: COC1CC(Nc2nccc(-c3cccc(-c4cc(C5(O)CCN(C)C5=O)on4)n3)n2)C1]

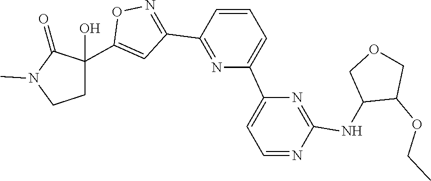 [SMILES: CCOC1COCC1Nc1nccc(-c2cccc(-c3cc(C4(O)CCN(C)C4=O)on3)n2)n1]

TABLE 1Q

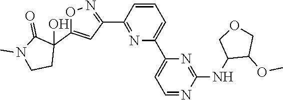

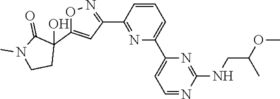

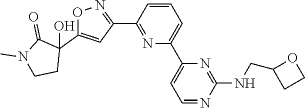 [SMILES: CN1CCC(O)(c2cc(-c3cccc(-c4ccnc(NCC5CCO5)n4)n3)no2)C1=O]

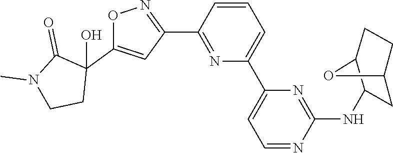 [SMILES: CN1CCC(O)(c2cc(-c3cccc(-c4ccnc(NC5CC6CCC5O6)n4)n3)no2)C1=O]

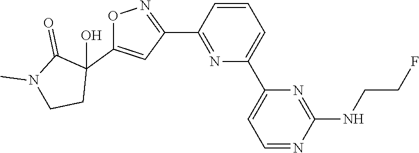 [SMILES: CN1CCC(O)(c2cc(-c3cccc(-c4ccnc(NCCF)n4)n3)no2)C1=O]

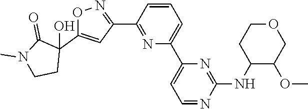

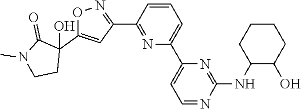 [SMILES: CN1CCC(O)(c2cc(-c3cccc(-c4ccnc(NC5CCCCC5O)n4)n3)no2)C1=O]

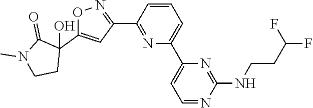 [SMILES: CN1CCC(O)(c2cc(-c3cccc(-c4ccnc(NCCC(F)F)n4)n3)no2)C1=O]

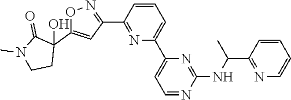 [SMILES: CC(Nc1nccc(-c2cccc(-c3cc(C4(O)CCN(C)C4=O)on3)n2)n1)c1ccccn1]

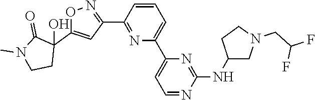 [SMILES: CN1CCC(O)(c2cc(-c3cccc(-c4ccnc(NC5CCN(CC(F)F)C5)n4)n3)no2)C1=O]

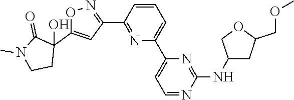

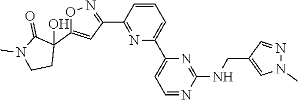

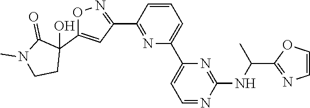 [SMILES: CC(Nc1nccc(-c2cccc(-c3cc(C4(O)CCN(C)C4=O)on3)n2)n1)c1ncco1]

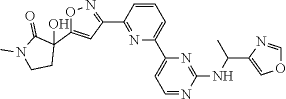 [SMILES: CC(Nc1nccc(-c2cccc(-c3cc(C4(O)CCN(C)C4=O)on3)n2)n1)c1cocn1]

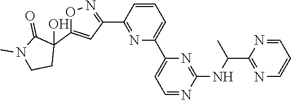 [SMILES: CC(Nc1nccc(-c2cccc(-c3cc(C4(O)CCN(C)C4=O)on3)n2)n1)c1ncccn1]

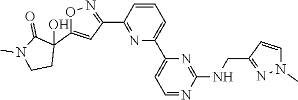

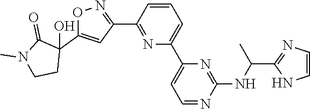 [SMILES: CC(Nc1nccc(-c2cccc(-c3cc(C4(O)CCN(C)C4=O)on3)n2)n1)c1ncc[nH]1]

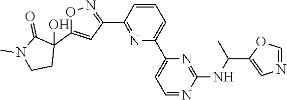 [SMILES: CC(Nc1nccc(-c2cccc(-c3cc(C4(O)CCN(C)C4=O)on3)n2)n1)c1cnco1]

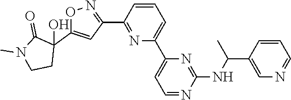 [SMILES: CC(Nc1nccc(-c2cccc(-c3cc(C4(O)CCN(C)C4=O)on3)n2)n1)c1cccnc1]

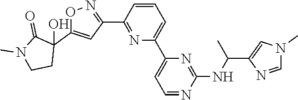

TABLE 1R

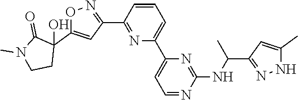

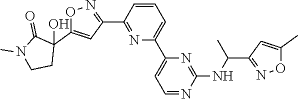

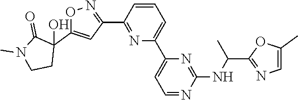

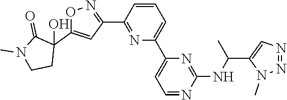 [SMILES: CC(Nc1nccc(-c2cccc(-c3cc(C4(O)CCN(C)C4=O)on3)n2)n1)c1cnnn1C]

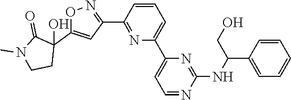 [SMILES: CN1CCC(O)(c2cc(-c3cccc(-c4ccnc(NC(CO)c5ccccc5)n4)n3)no2)C1=O]

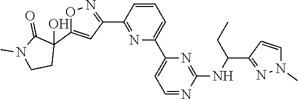

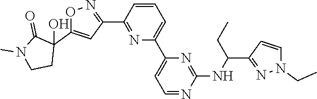 [SMILES: CCC(Nc1nccc(-c2cccc(-c3cc(C4(O)CCN(C)C4=O)on3)n2)n1)c1ccn(CC)n1]

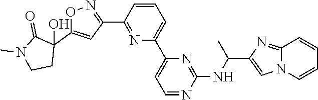 [SMILES: CC(Nc1nccc(-c2cccc(-c3cc(C4(O)CCN(C)C4=O)on3)n2)n1)c1cn2ccccc2n1]

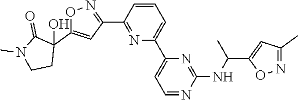

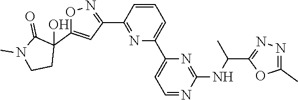

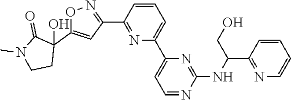 [SMILES: CN1CCC(O)(c2cc(-c3cccc(-c4ccnc(NC(CO)c5ccccn5)n4)n3)no2)C1=O]

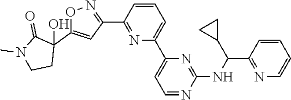 [SMILES: CN1CCC(O)(c2cc(-c3cccc(-c4ccnc(NC(c5ccccn5)C5CC5)n4)n3)no2)C1=O]

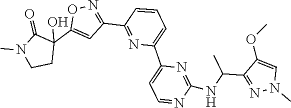

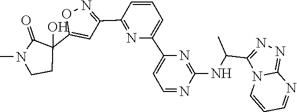 [SMILES: CC(Nc1nccc(-c2cccc(-c3cc(C4(O)CCN(C)C4=O)on3)n2)n1)c1nnc2ncccn12]

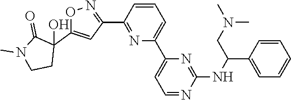 [SMILES: CN(C)CC(Nc1nccc(-c2cccc(-c3cc(C4(O)CCN(C)C4=O)on3)n2)n1)c1ccccc1]

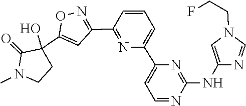 [SMILES: CN1CCC(O)(c2cc(-c3cccc(-c4ccnc(Nc5cn(CCF)cn5)n4)n3)no2)C1=O]

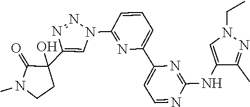

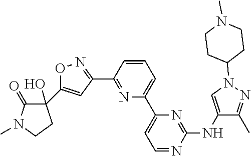

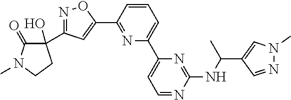

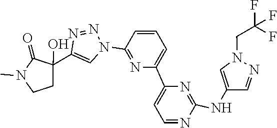 [SMILES: CN1CCC(O)(c2cn(-c3cccc(-c4ccnc(Nc5cnn(CC(F)(F)F)c5)n4)n3)nn2)C1=O]

TABLE 1S

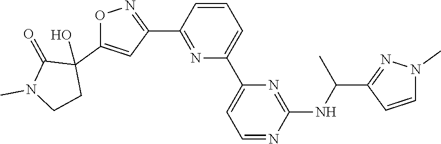

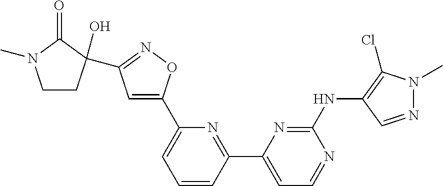

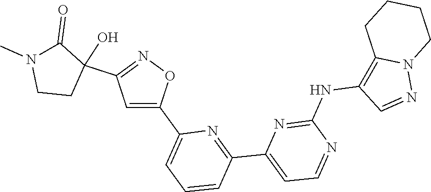 [SMILES: CN1CCC(O)(c2cc(-c3cccc(-c4ccnc(Nc5cnn6c5CCCC6)n4)n3)on2)C1=O]

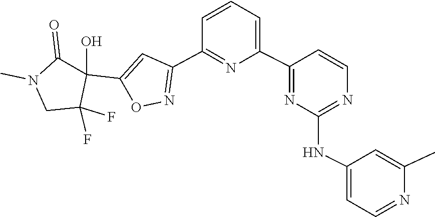

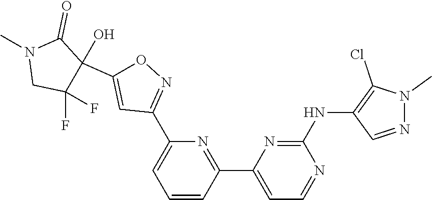

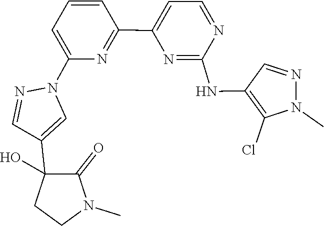 [SMILES: CN1CCC(O)(c2cnn(-c3cccc(-c4ccnc(Nc5cnn(C)c5Cl)n4)n3)c2)C1=O]

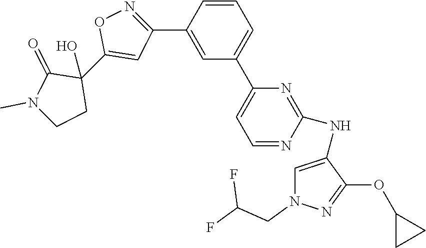 [SMILES: CN1CCC(O)(c2cc(-c3cccc(-c4ccnc(Nc5cn(CC(F)F)nc5OC5CC5)n4)c3)no2)C1=O]

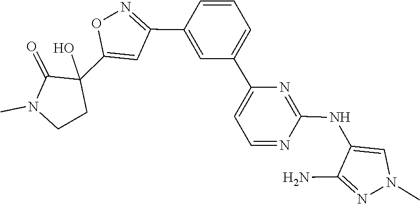

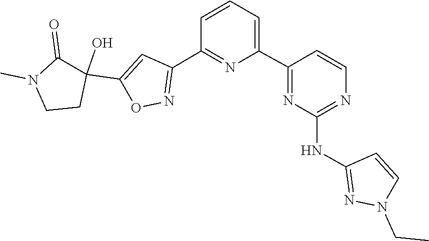 [SMILES: CCn1ccc(Nc2nccc(-c3cccc(-c4cc(C5(O)CCN(C)C5=O)on4)n3)n2)n1]

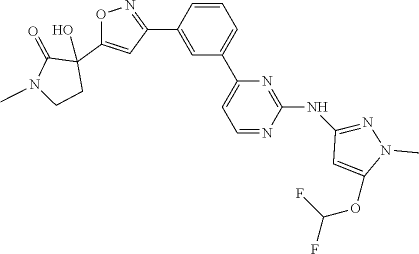

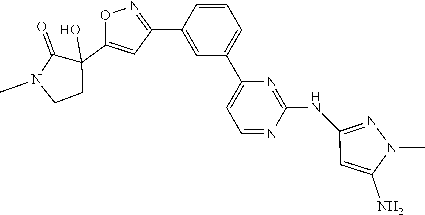

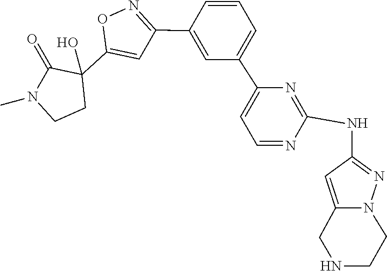 [SMILES: CN1CCC(O)(c2cc(-c3cccc(-c4ccnc(Nc5cc6n(n5)CCNC6)n4)c3)no2)C1=O]

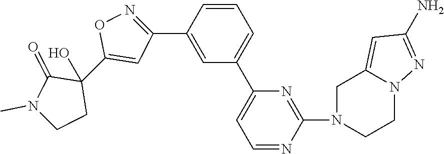 [SMILES: CN1CCC(O)(c2cc(-c3cccc(-c4ccnc(N5CCn6nc(N)cc6C5)n4)c3)no2)C1=O]

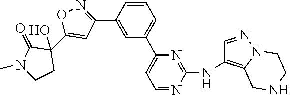 [SMILES: CN1CCC(O)(c2cc(-c3cccc(-c4ccnc(Nc5cnn6c5CNCC6)n4)c3)no2)C1=O]

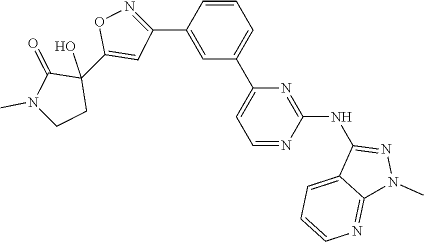

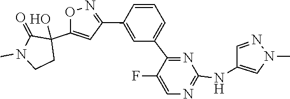

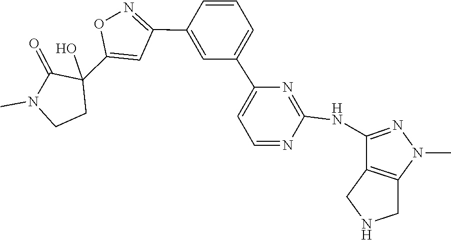

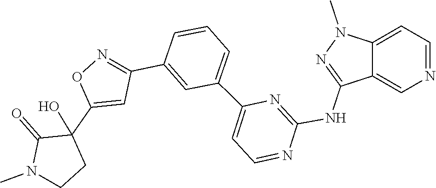 [SMILES: CN1CCC(O)(c2cc(-c3cccc(-c4ccnc(Nc5nn(C)c6ccncc56)n4)c3)no2)C1=O]

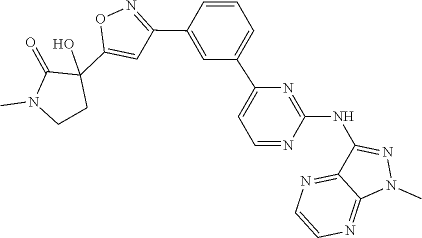

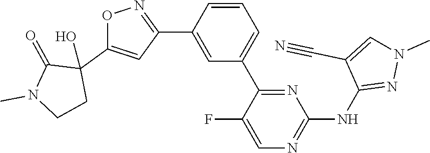

TABLE 1T

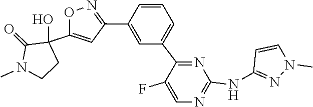

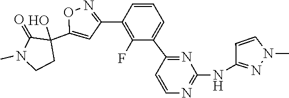

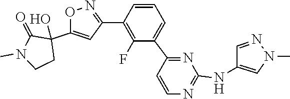

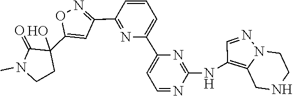 [SMILES: CN1CCC(O)(c2cc(-c3cccc(-c4ccnc(Nc5cnn6c5CNCC6)n4)n3)no2)C1=O]

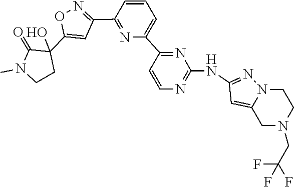 [SMILES: CN1CCC(O)(c2cc(-c3cccc(-c4ccnc(Nc5cc6n(n5)CCN(CC(F)(F)F)C6)n4)n3)no2)C1=O]

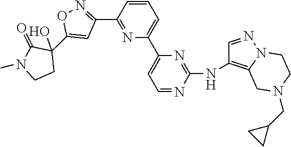 [SMILES: CN1CCC(O)(c2cc(-c3cccc(-c4ccnc(Nc5cnn6c5CN(CC5CC5)CC6)n4)n3)no2)C1=O]

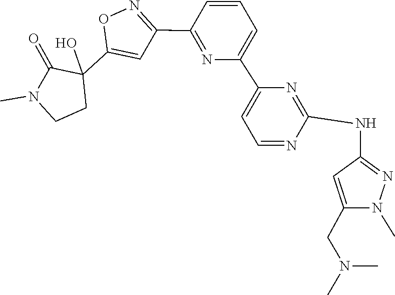

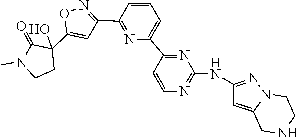 [SMILES: CN1CCC(O)(c2cc(-c3cccc(-c4ccnc(Nc5cc6n(n5)CCNC6)n4)n3)no2)C1=O]

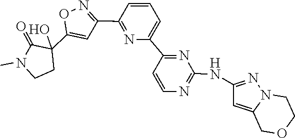 [SMILES: CN1CCC(O)(c2cc(-c3cccc(-c4ccnc(Nc5cc6n(n5)CCOC6)n4)n3)no2)C1=O]

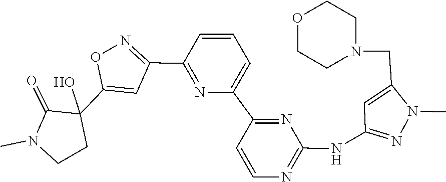

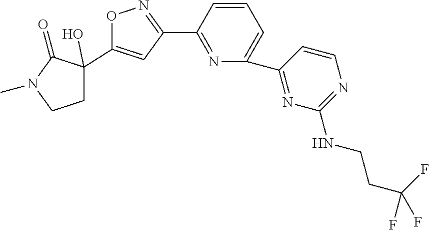 [SMILES: CN1CCC(O)(c2cc(-c3cccc(-c4ccnc(NCCC(F)(F)F)n4)n3)no2)C1=O]

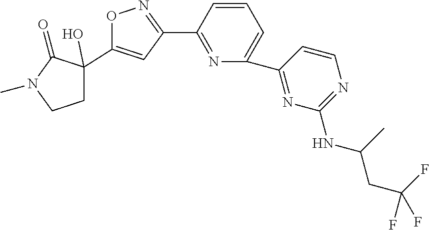 [SMILES: CC(CC(F)(F)F)Nc1nccc(-c2cccc(-c3cc(C4(O)CCN(C)C4=O)on3)n2)n1]

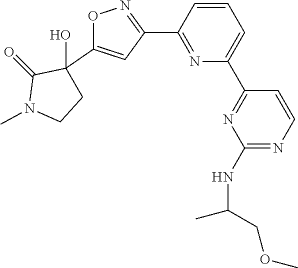

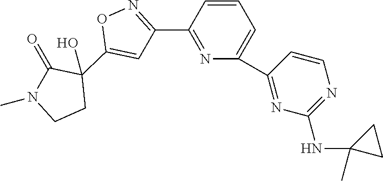 [SMILES: CN1CCC(O)(c2cc(-c3cccc(-c4ccnc(NC5(C)CC5)n4)n3)no2)C1=O]

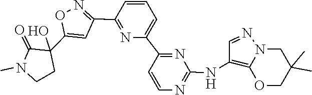

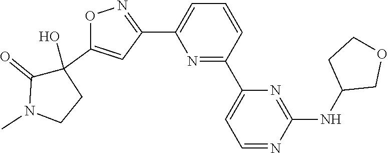 [SMILES: CN1CCC(O)(c2cc(-c3cccc(-c4ccnc(NC5CCOC5)n4)n3)no2)C1=O]

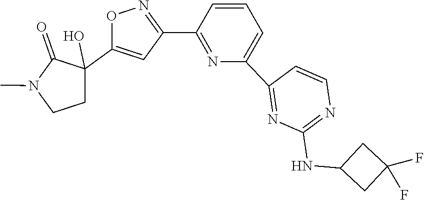 [SMILES: CN1CCC(O)(c2cc(-c3cccc(-c4ccnc(NC5CC(F)(F)C5)n4)n3)no2)C1=O]

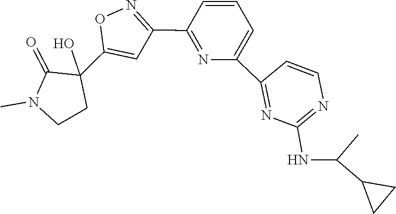 [SMILES: CC(Nc1nccc(-c2cccc(-c3cc(C4(O)CCN(C)C4=O)on3)n2)n1)C1CC1]

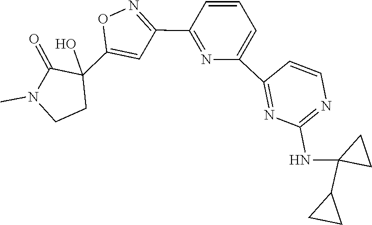 [SMILES: CN1CCC(O)(c2cc(-c3cccc(-c4ccnc(NC5(C6CC6)CC5)n4)n3)no2)C1=O]

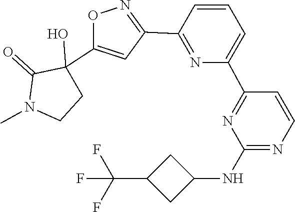 [SMILES: CN1CCC(O)(c2cc(-c3cccc(-c4ccnc(NC5CC(C(F)(F)F)C5)n4)n3)no2)C1=O]

TABLE 1U

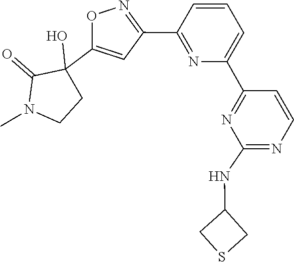 [SMILES: CN1CCC(O)(c2cc(-c3cccc(-c4ccnc(NC5CSC5)n4)n3)no2)C1=O]

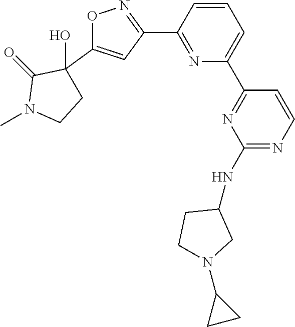 [SMILES: CN1CCC(O)(c2cc(-c3cccc(-c4ccnc(NC5CCN(C6CC6)C5)n4)n3)no2)C1=O]

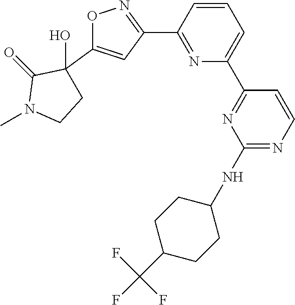 [SMILES: CN1CCC(O)(c2cc(-c3cccc(-c4ccnc(NC5CCC(C(F)(F)F)CC5)n4)n3)no2)C1=O]

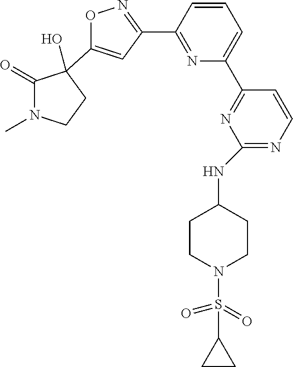 [SMILES: CN1CCC(O)(c2cc(-c3cccc(-c4ccnc(NC5CCN(S(=O)(=O)C6CC6)CC5)n4)n3)no2)C1=O]

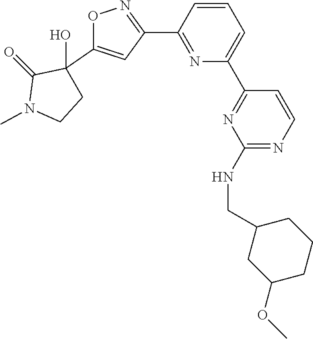 [SMILES: COC1CCCC(CNc2nccc(-c3cccc(-c4cc(C5(O)CCN(C)C5=O)on4)n3)n2)C1]

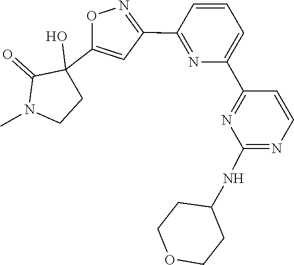 [SMILES: CN1CCC(O)(c2cc(-c3cccc(-c4ccnc(NC5CCOCC5)n4)n3)no2)C1=O]

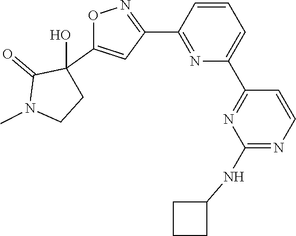 [SMILES: CN1CCC(O)(c2cc(-c3cccc(-c4ccnc(NC5CCC5)n4)n3)no2)C1=O]

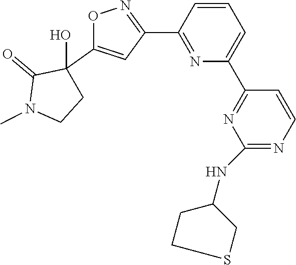 [SMILES: CN1CCC(O)(c2cc(-c3cccc(-c4ccnc(NC5CCSC5)n4)n3)no2)C1=O]

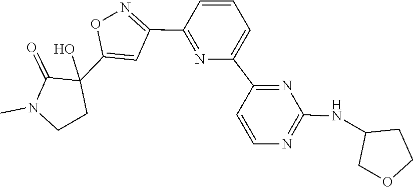 [SMILES: CN1CCC(O)(c2cc(-c3cccc(-c4ccnc(NC5CCOC5)n4)n3)no2)C1=O]

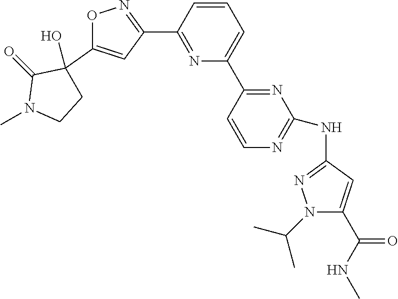 [SMILES: CNC(=O)c1cc(Nc2nccc(-c3cccc(-c4cc(C5(O)CCN(C)C5=O)on4)n3)n2)nn1C(C)C]

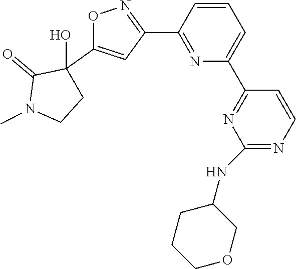 [SMILES: CN1CCC(O)(c2cc(-c3cccc(-c4ccnc(NC5CCCOC5)n4)n3)no2)C1=O]

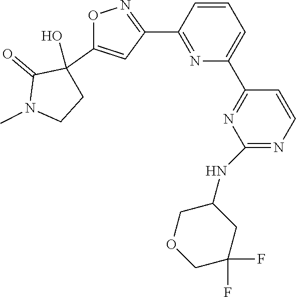 [SMILES: CN1CCC(O)(c2cc(-c3cccc(-c4ccnc(NC5COCC(F)(F)C5)n4)n3)no2)C1=O]

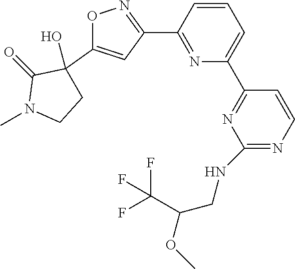 [SMILES: COC(CNc1nccc(-c2cccc(-c3cc(C4(O)CCN(C)C4=O)on3)n2)n1)C(F)(F)F]

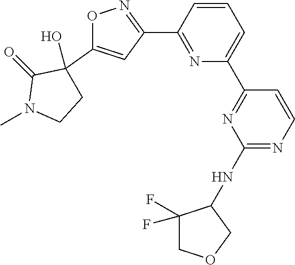 [SMILES: CN1CCC(O)(c2cc(-c3cccc(-c4ccnc(NC5COCC5(F)F)n4)n3)no2)C1=O]

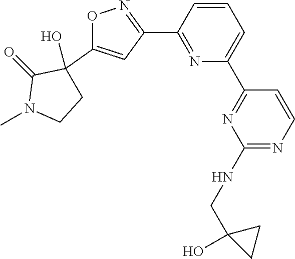 [SMILES: CN1CCC(O)(c2cc(-c3cccc(-c4ccnc(NCC5(O)CC5)n4)n3)no2)C1=O]

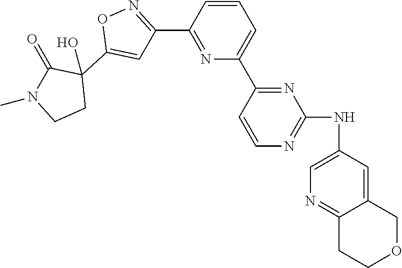 [SMILES: CN1CCC(O)(c2cc(-c3cccc(-c4ccnc(Nc5cnc6c(c5)COCC6)n4)n3)no2)C1=O]

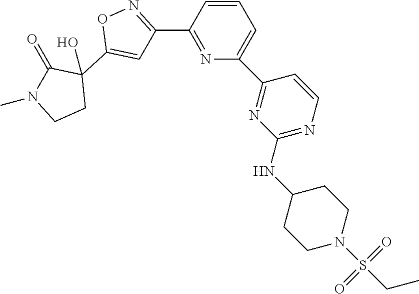 [SMILES: CCS(=O)(=O)N1CCC(Nc2nccc(-c3cccc(-c4cc(C5(O)CCN(C)C5=O)on4)n3)n2)CC1]

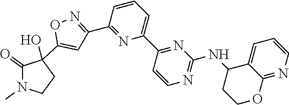 [SMILES: CN1CCC(O)(c2cc(-c3cccc(-c4ccnc(NC5CCOc6ncccc65)n4)n3)no2)C1=O]

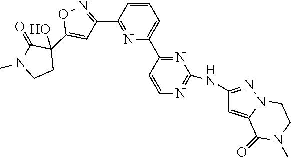 [SMILES: CN1CCn2nc(Nc3nccc(-c4cccc(-c5cc(C6(O)CCN(C)C6=O)on5)n4)n3)cc2C1=O]

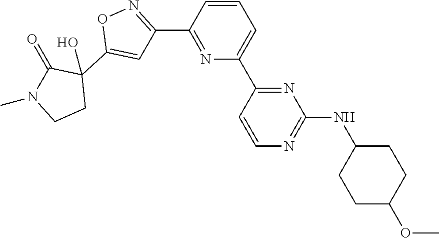

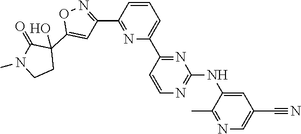 [SMILES: Cc1ncc(C#N)cc1Nc1nccc(-c2cccc(-c3cc(C4(O)CCN(C)C4=O)on3)n2)n1]

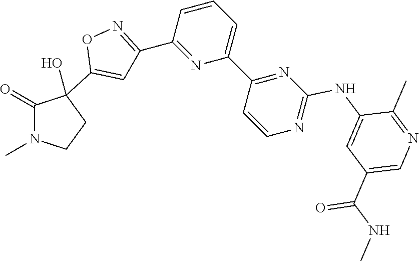 [SMILES: CNC(=O)c1cnc(C)c(Nc2nccc(-c3cccc(-c4cc(C5(O)CCN(C)C5=O)on4)n3)n2)c1]

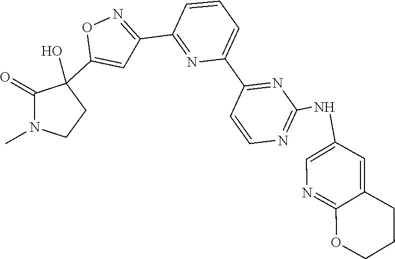 [SMILES: CN1CCC(O)(c2cc(-c3cccc(-c4ccnc(Nc5cnc6c(c5)CCCO6)n4)n3)no2)C1=O]

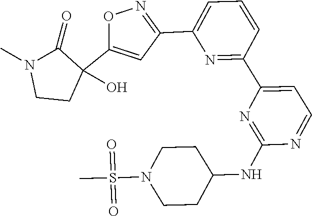 [SMILES: CN1CCC(O)(c2cc(-c3cccc(-c4ccnc(NC5CCN(S(C)(=O)=O)CC5)n4)n3)no2)C1=O]

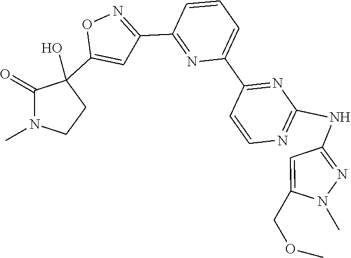 [SMILES: COCc1cc(Nc2nccc(-c3cccc(-c4cc(C5(O)CCN(C)C5=O)on4)n3)n2)nn1C]

In some embodiments, disclosed herein is a compound of Formula I′ or Formula I or a pharmaceutically acceptable salt thereof, having a structure as shown in any one of Tables 2A to 2D.

TABLE 2A

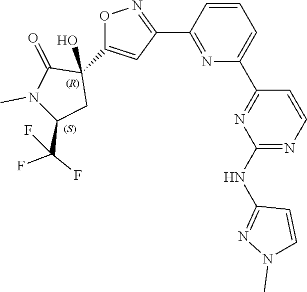

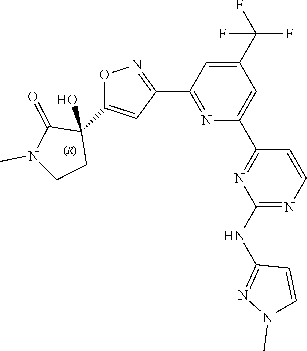

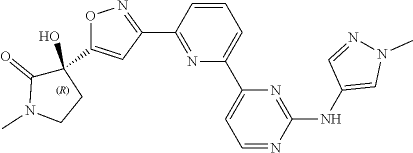

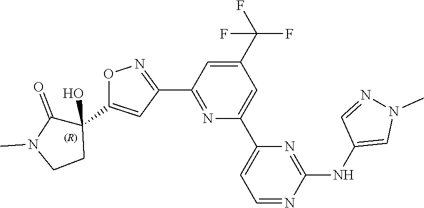

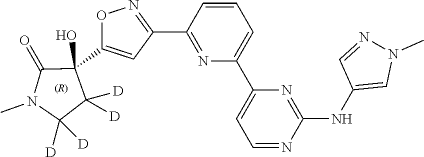

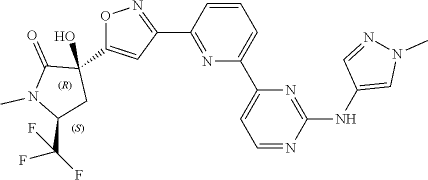

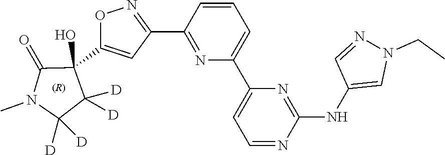

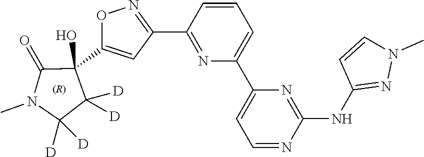

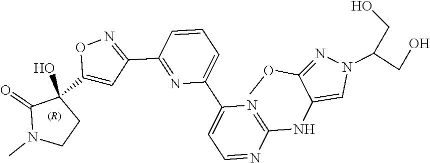

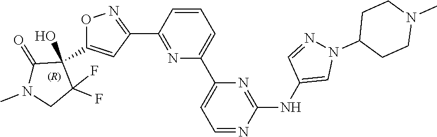

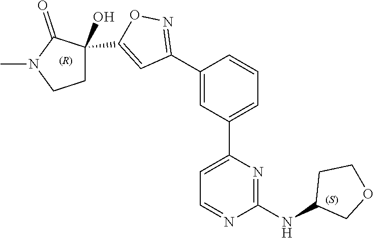

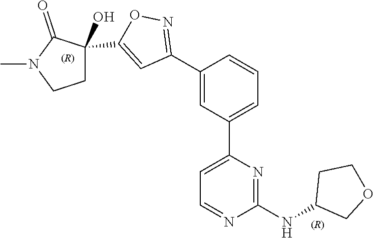

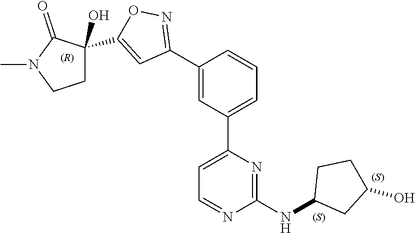

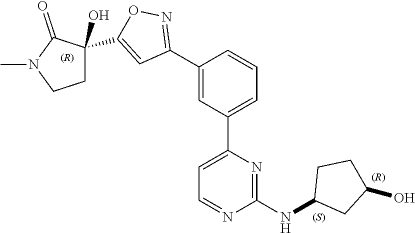

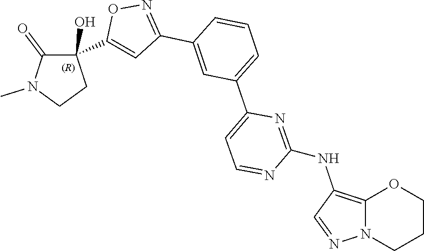

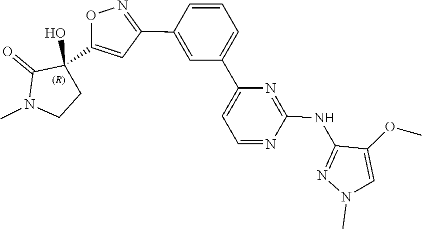

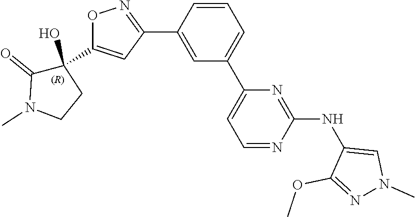

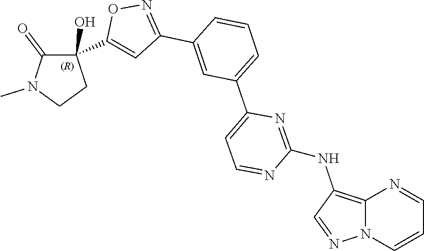

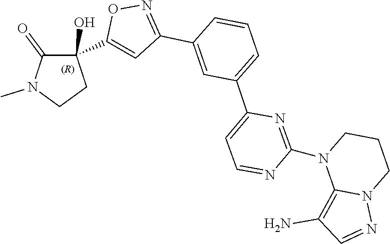

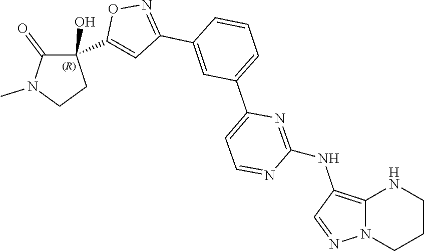

TABLE 2B

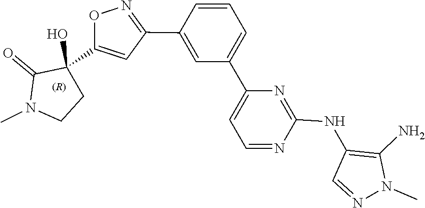

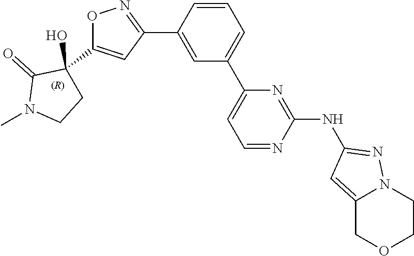

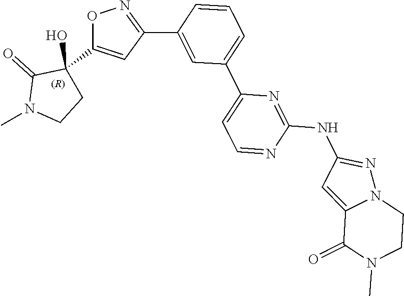

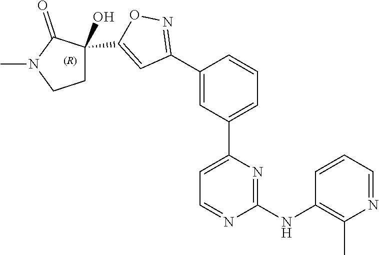

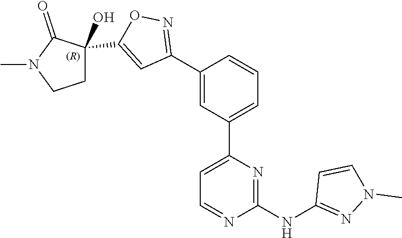

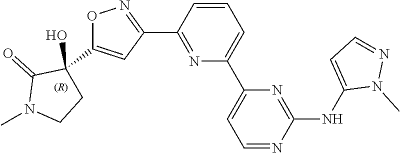

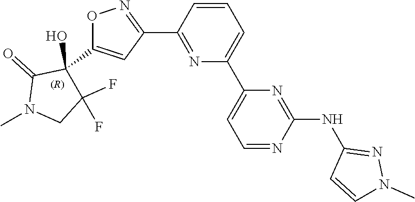

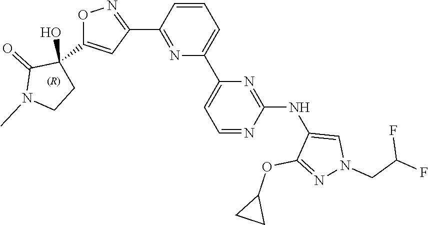

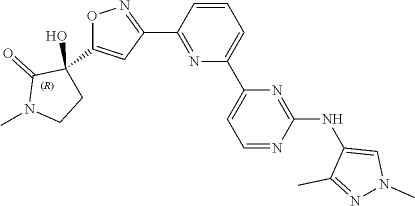

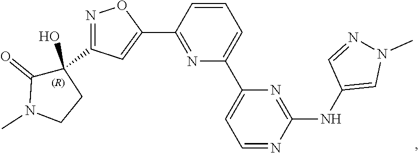

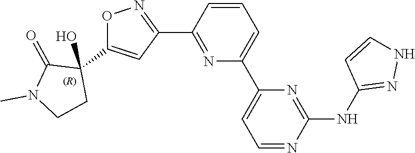

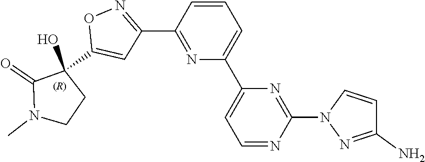

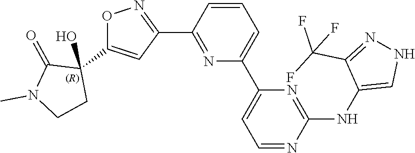

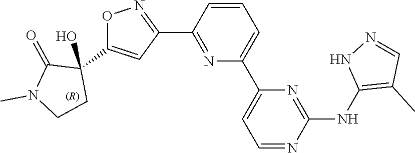

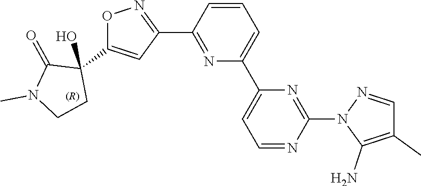

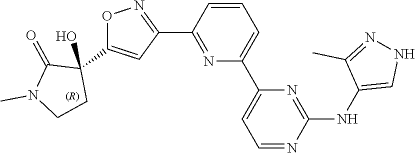

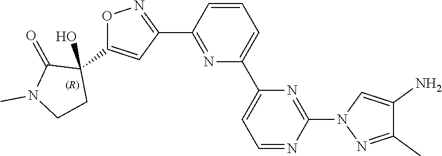

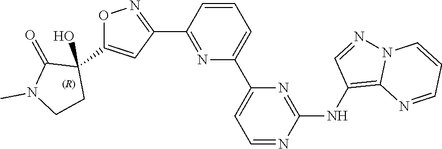

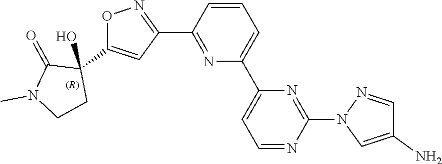

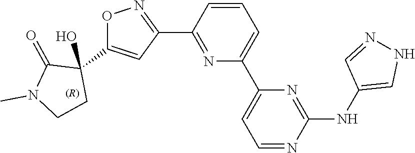

TABLE 2C

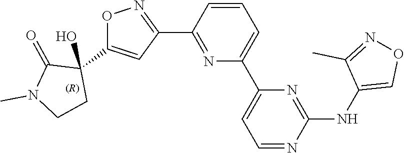

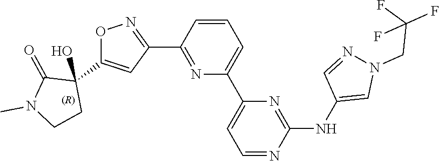

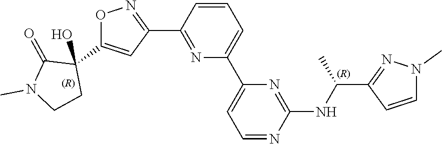

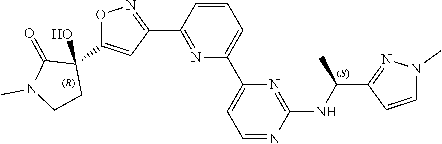

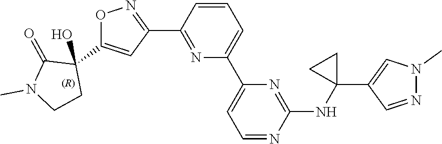

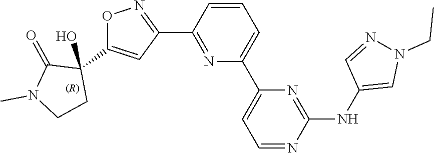

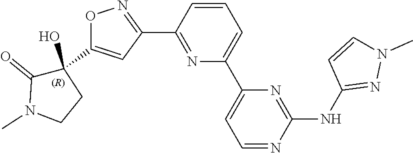

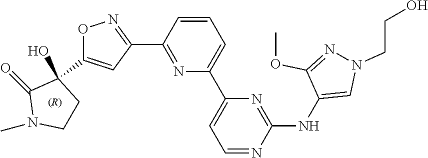

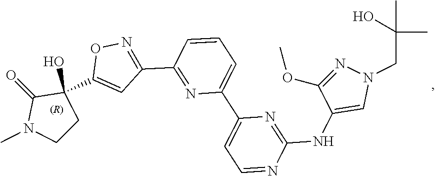

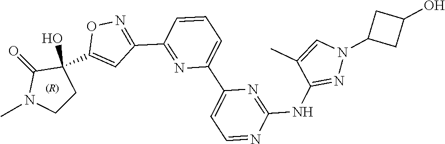

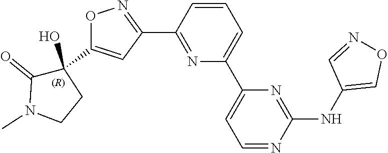

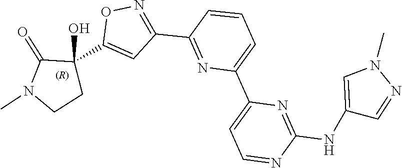

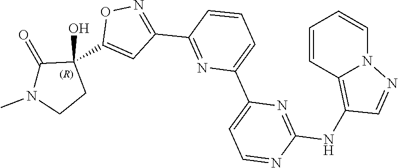

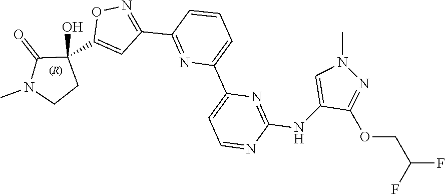

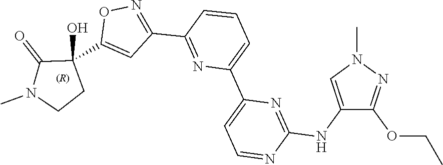

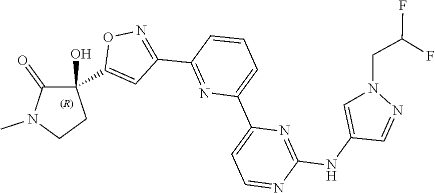

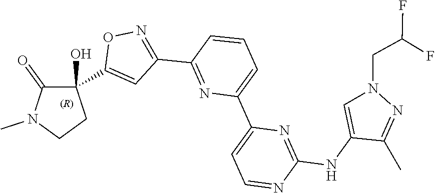

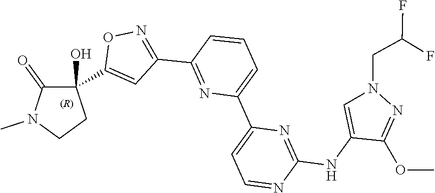

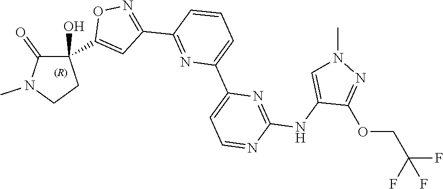

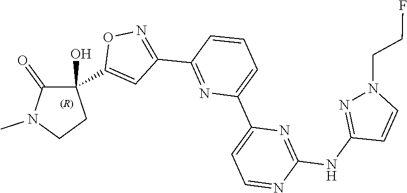

TABLE 2D

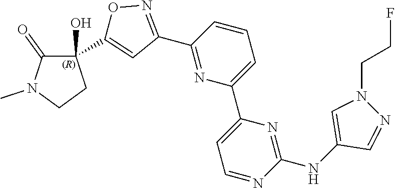

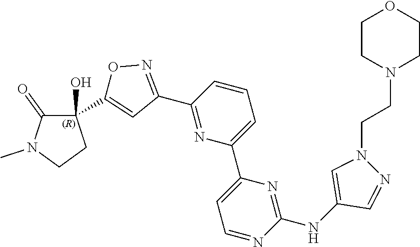

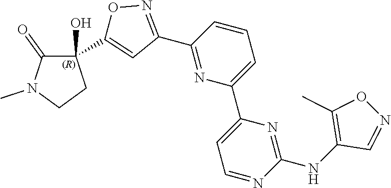

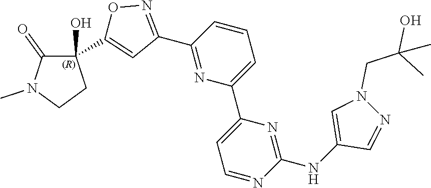

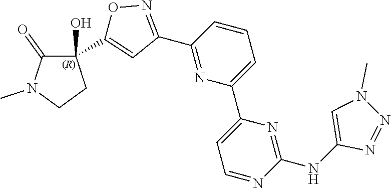

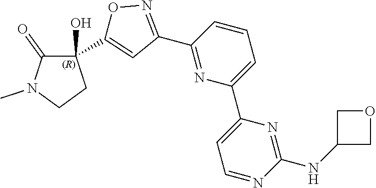

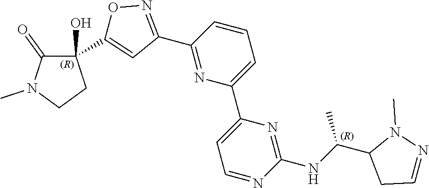

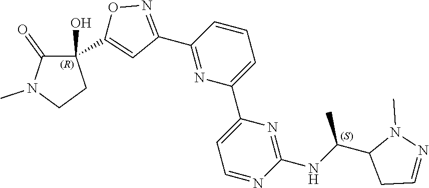

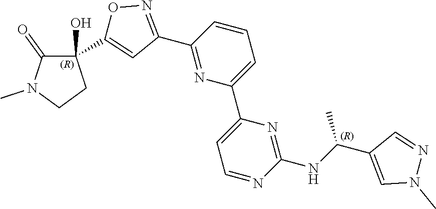

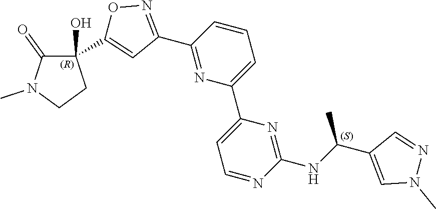

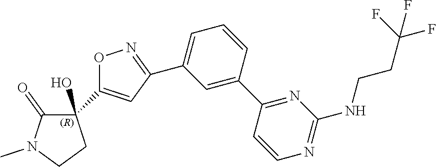

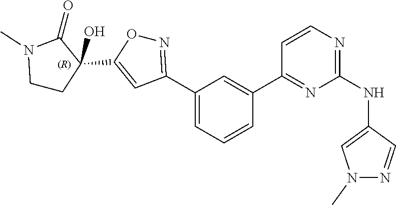

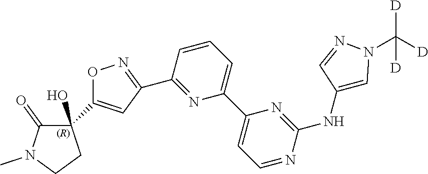

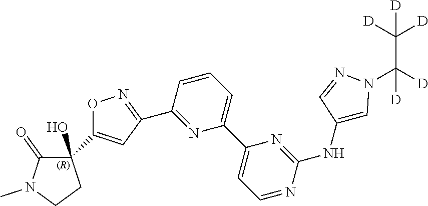

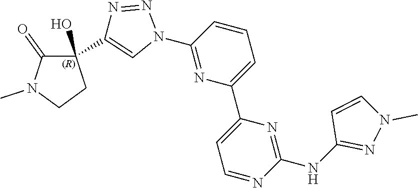

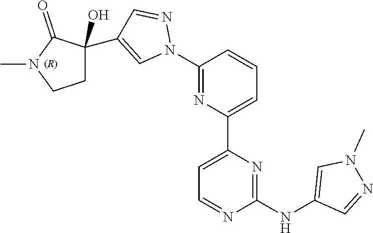

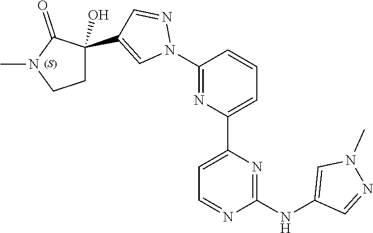

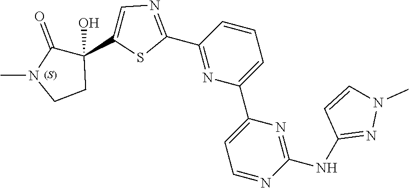

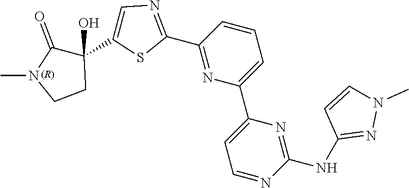

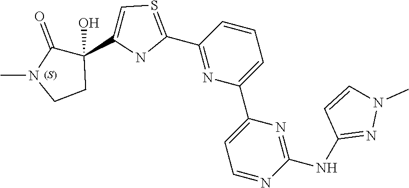

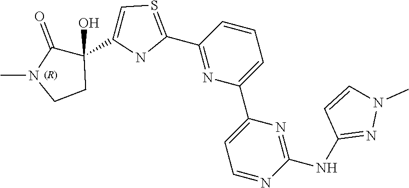

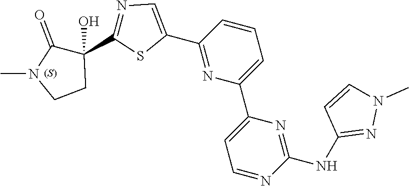

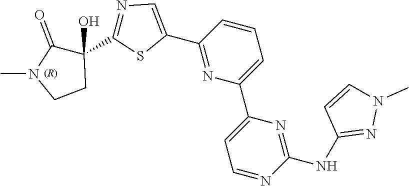

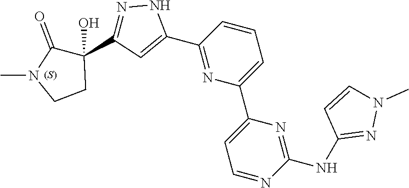

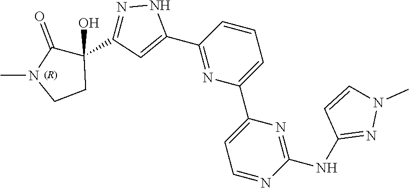

In some embodiments, disclosed herein is a compound, or a pharmaceutically acceptable salt thereof, having a structure as shown in any one of Tables 2E to 2T.

TABLE 2E

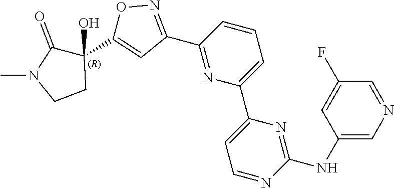

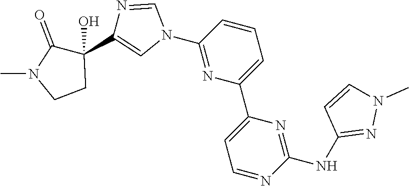

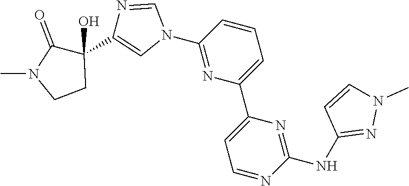

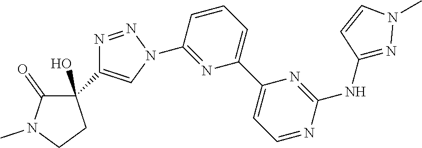

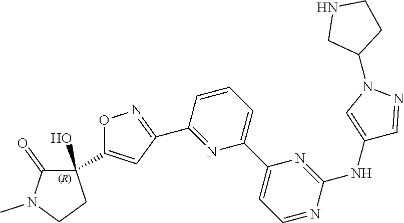

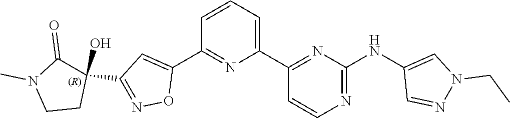

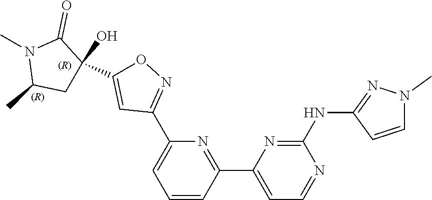

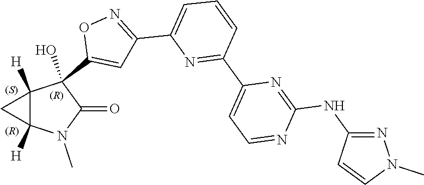

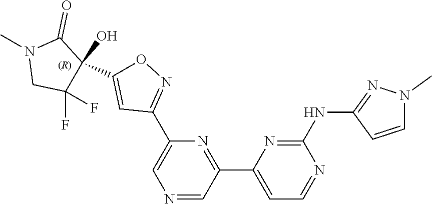

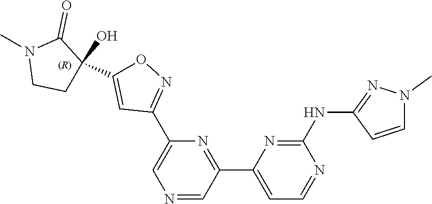

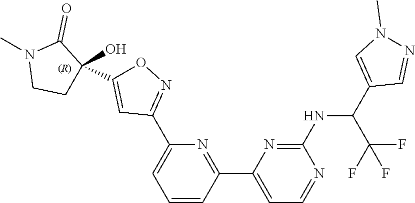

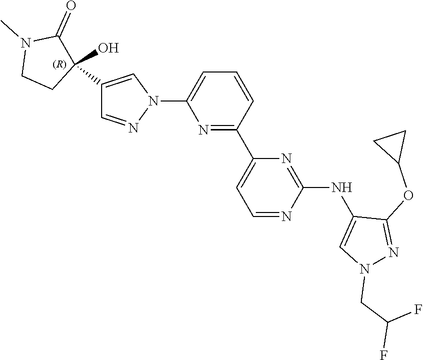

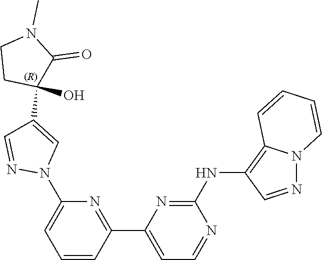

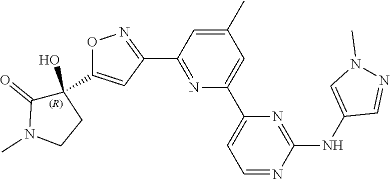

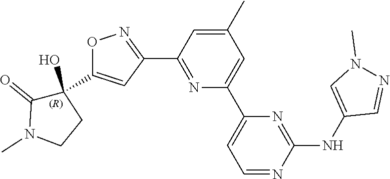

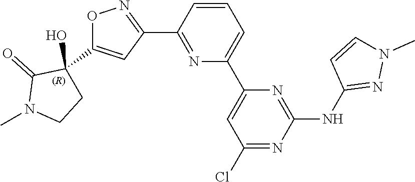

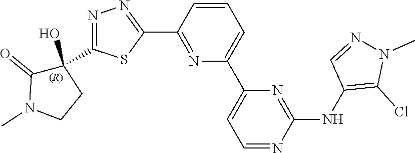

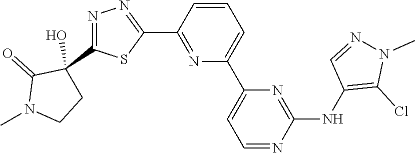

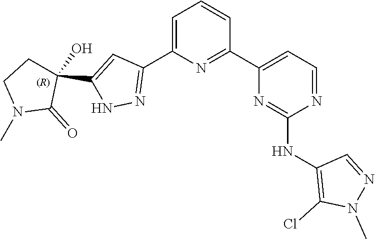

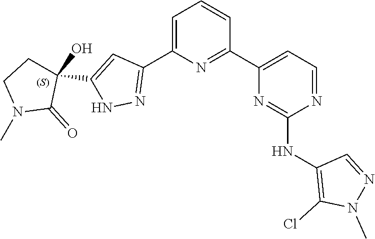

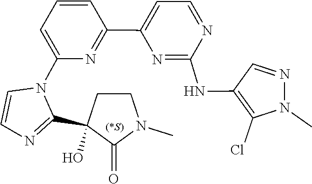

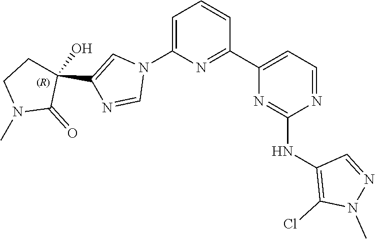

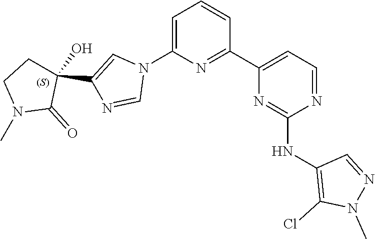

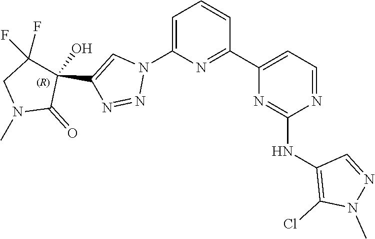

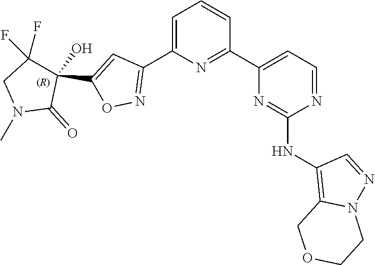

TABLE 2F

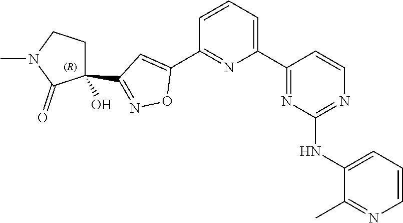

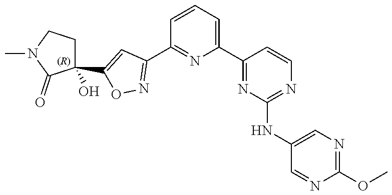

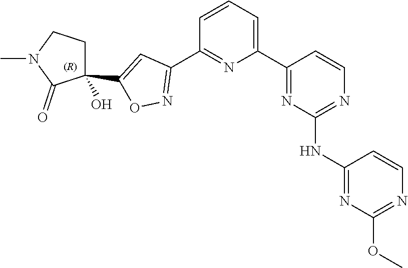

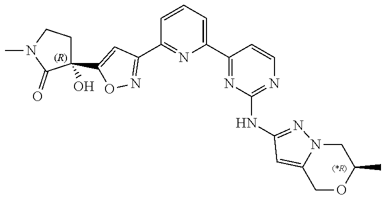

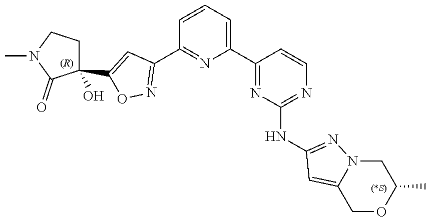

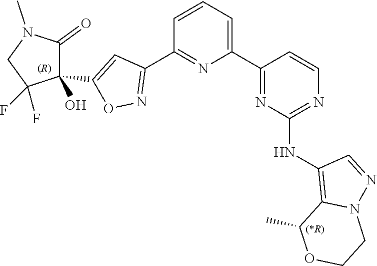

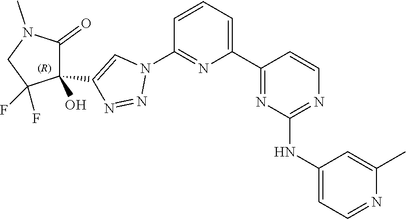

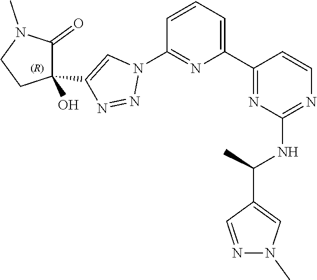

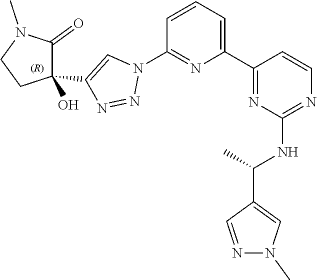

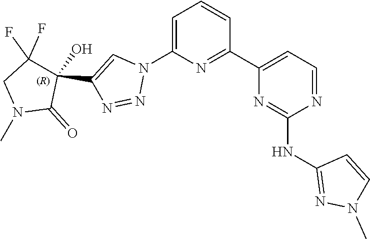

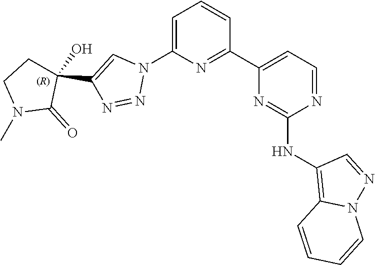

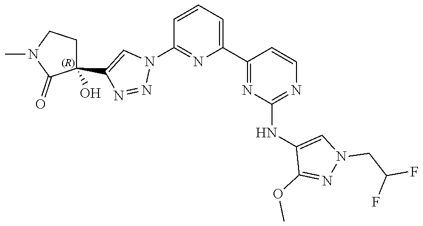

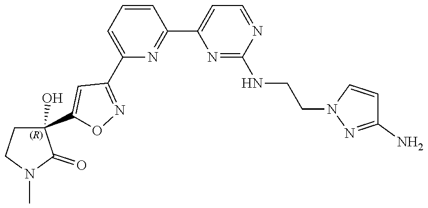

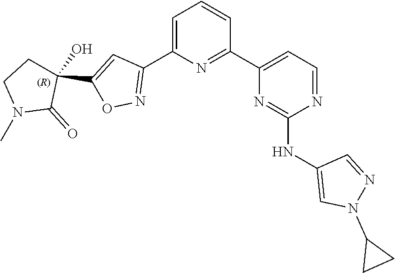

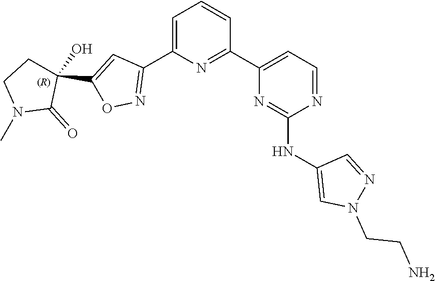

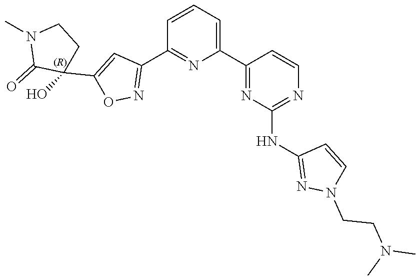

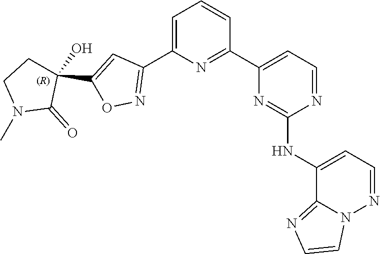

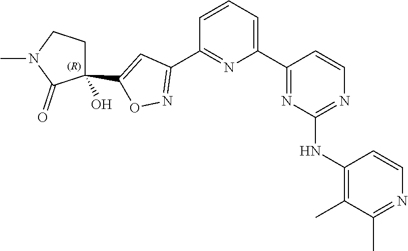

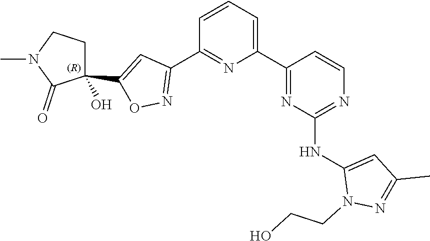

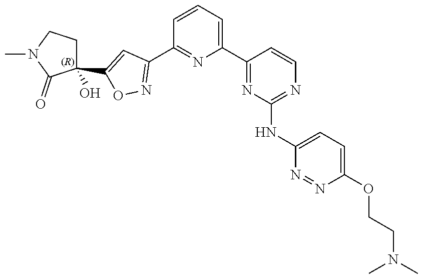

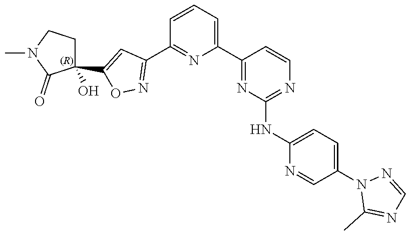

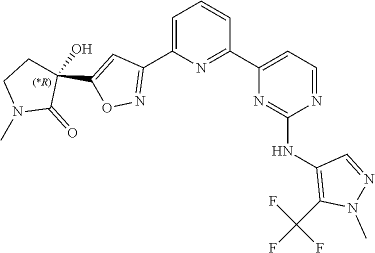

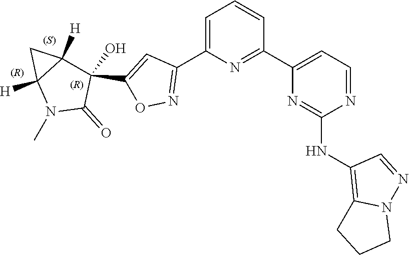

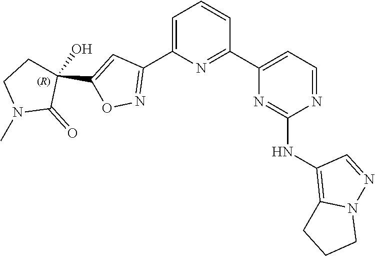

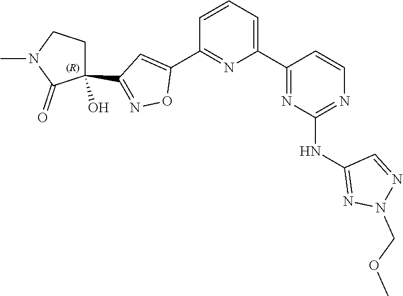

TABLE 2G

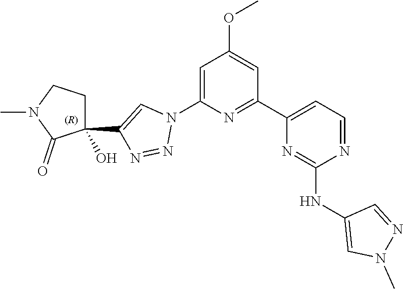

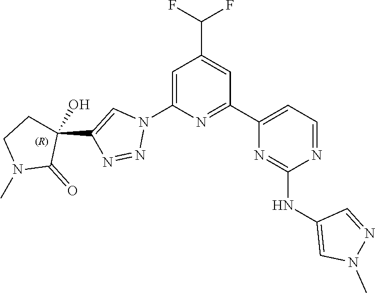

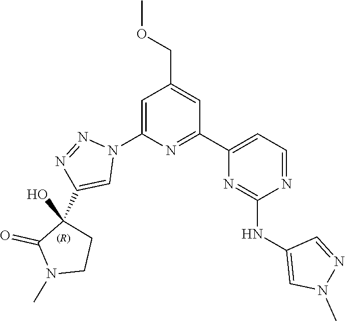

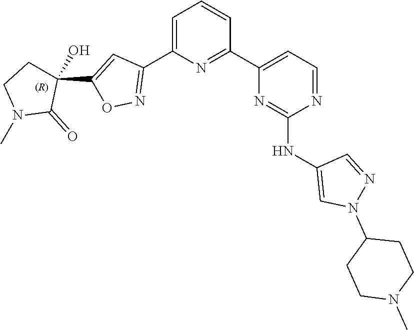

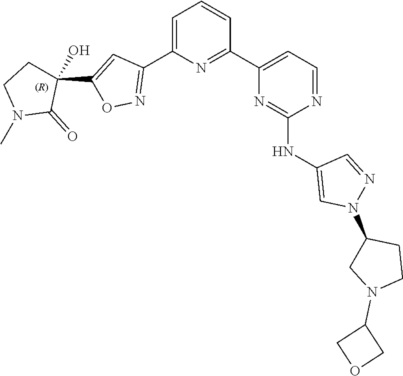

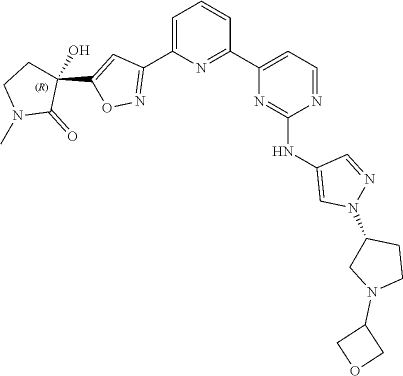

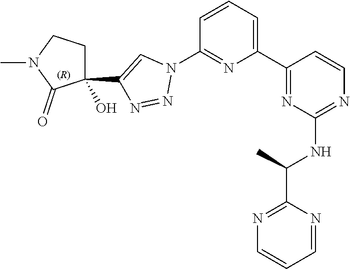

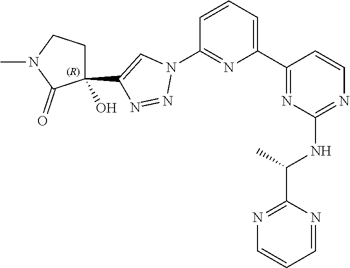

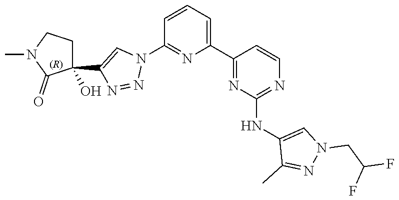

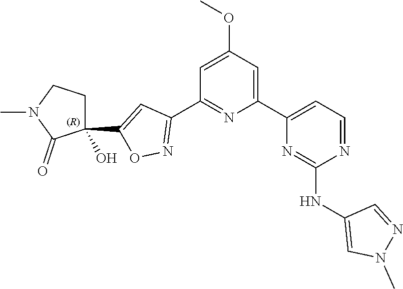

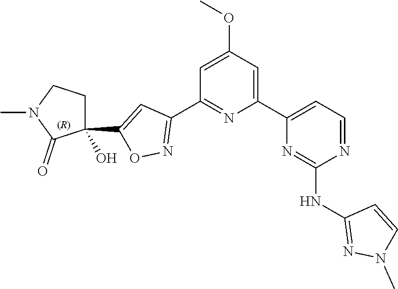

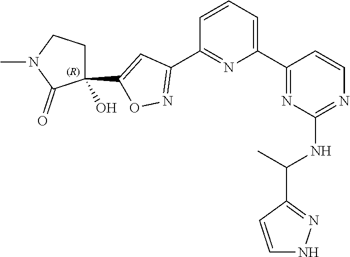

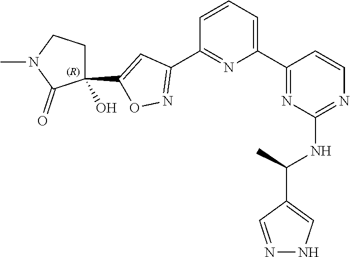

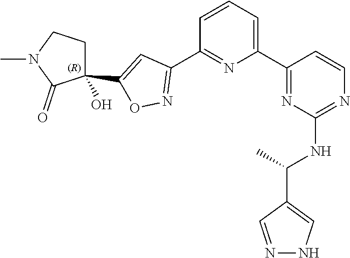

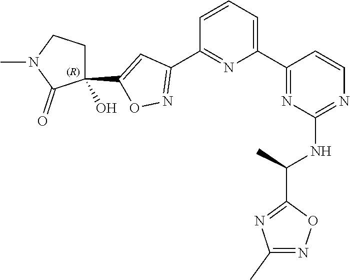

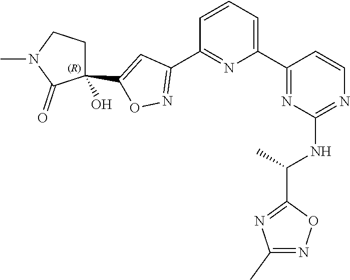

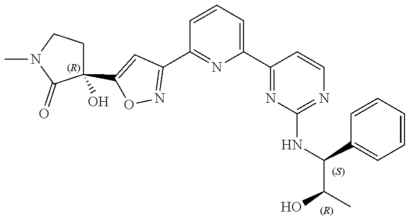

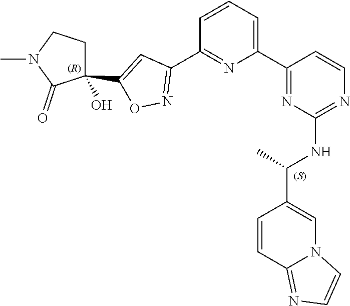

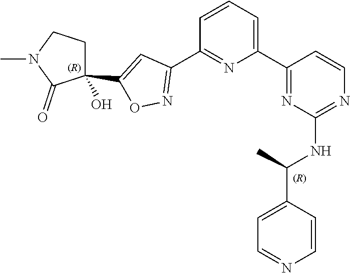

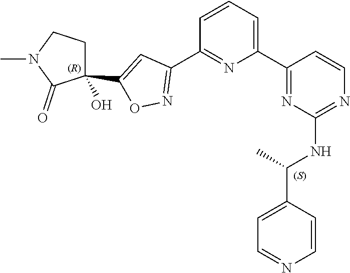

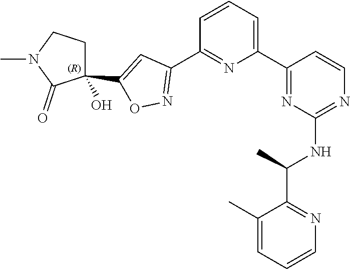

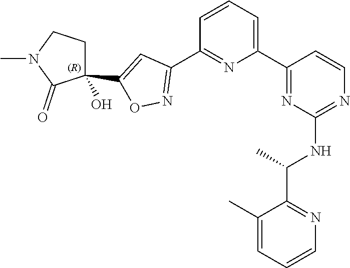

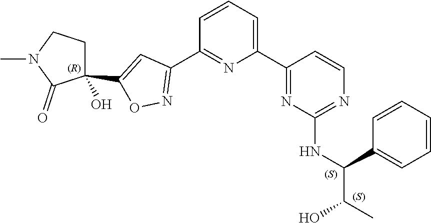

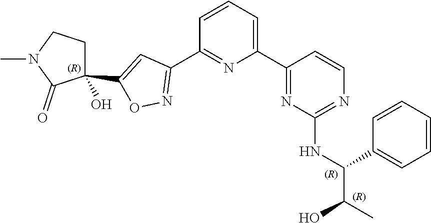

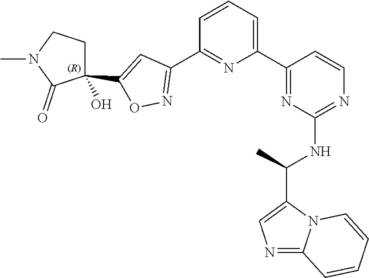

TABLE 2H

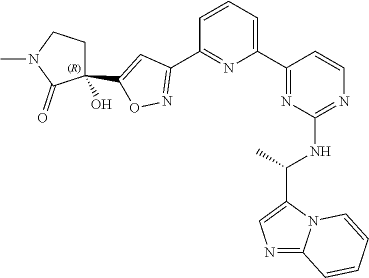

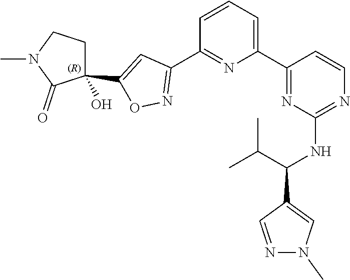

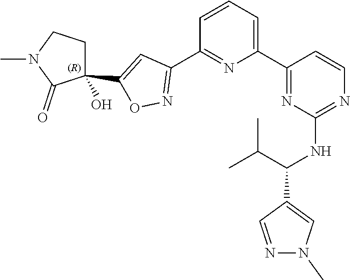

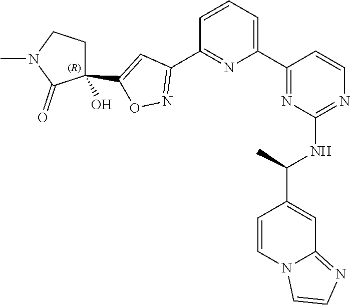

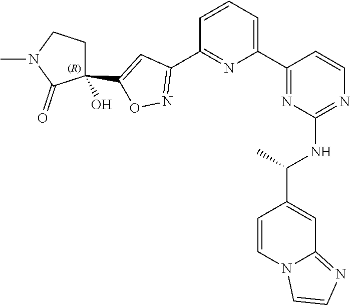

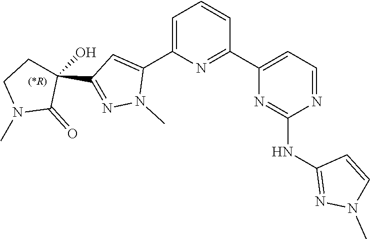 [SMILES: CN1CC[C@@](O)(c2cc(-c3cccc(-c4ccnc(Nc5ccn(C)n5)n4)n3)n(C)n2)C1=O]

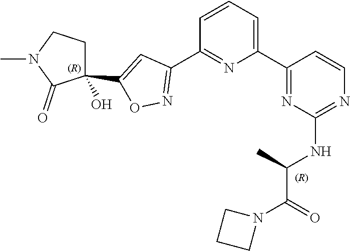

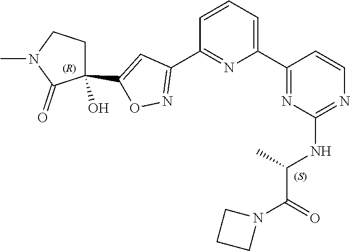

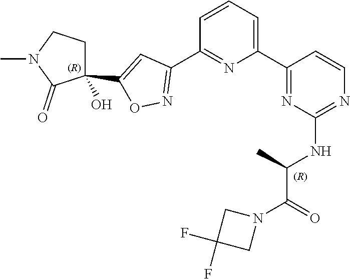

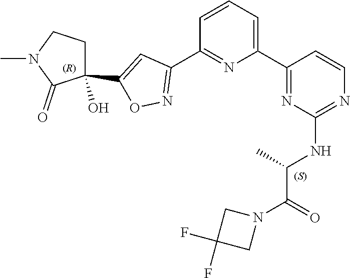

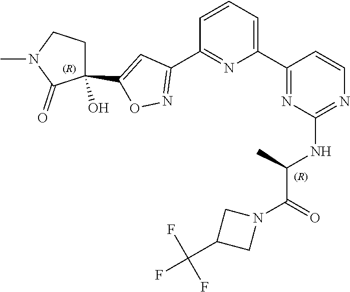

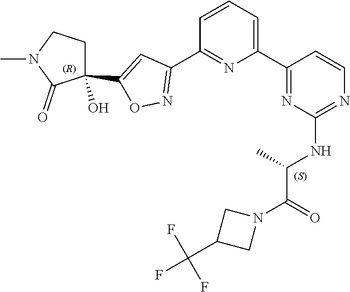

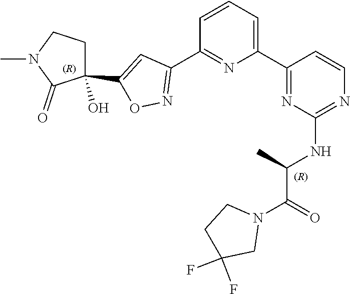

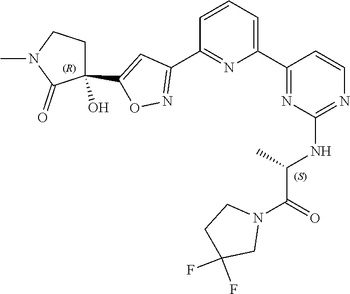

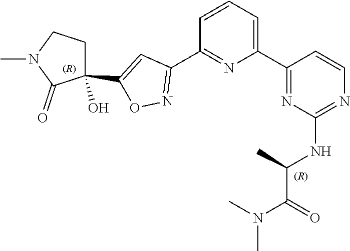

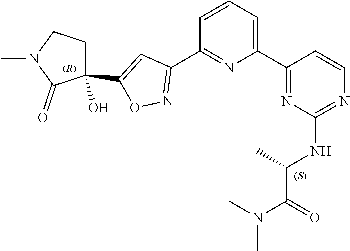

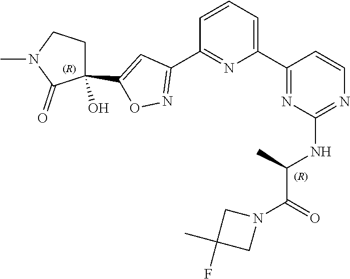

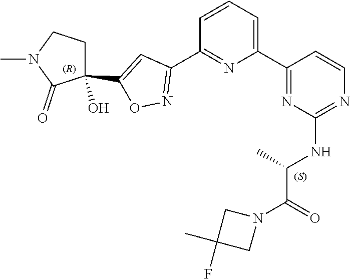

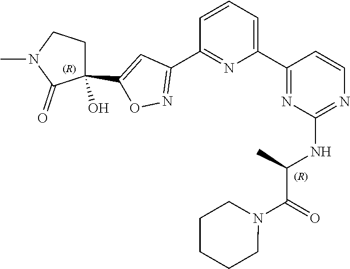

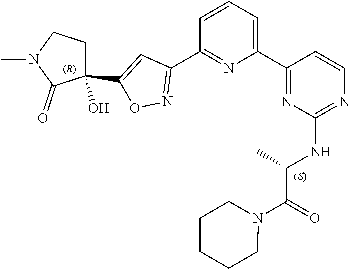

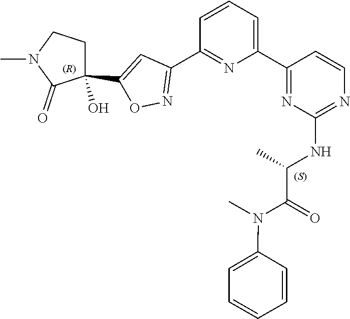

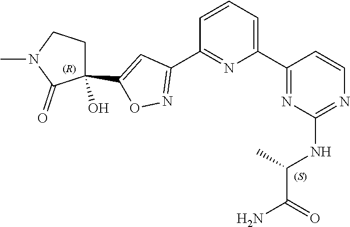

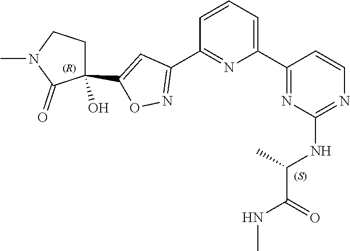

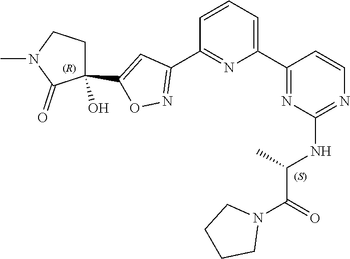

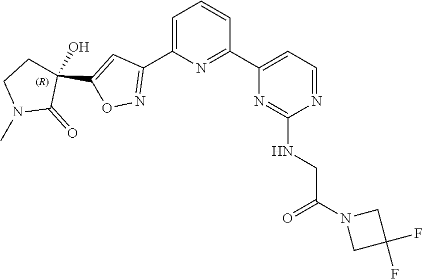

TABLE 2I

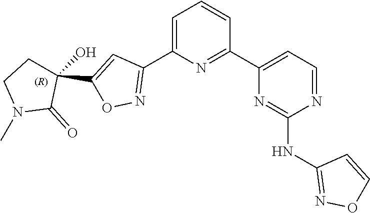

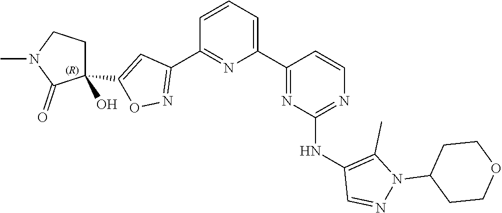

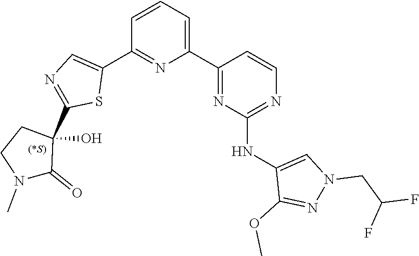 [SMILES: COc1nn(CC(F)F)cc1Nc1nccc(-c2cccc(-c3cnc([C@]4(O)CCN(C)C4=O)s3)n2)n1]

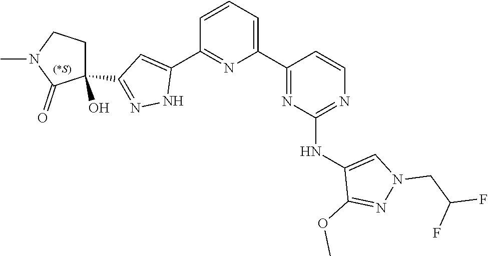

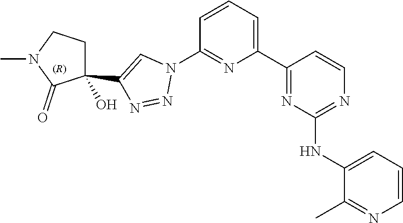

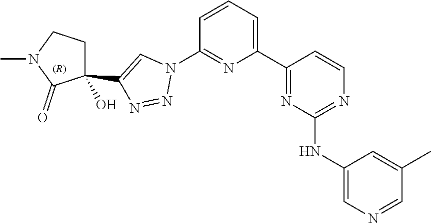

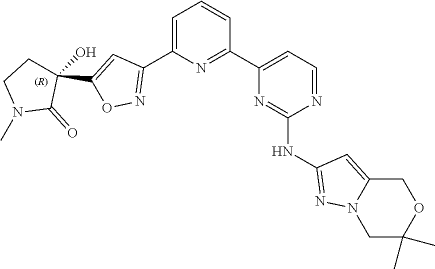

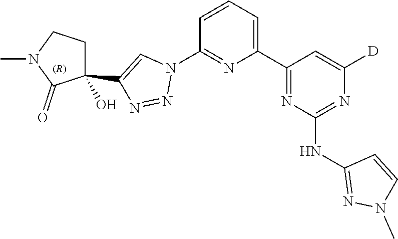

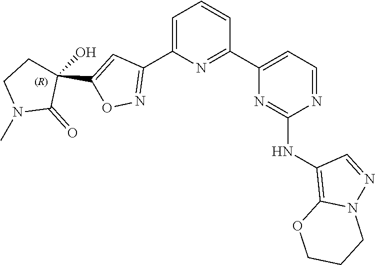

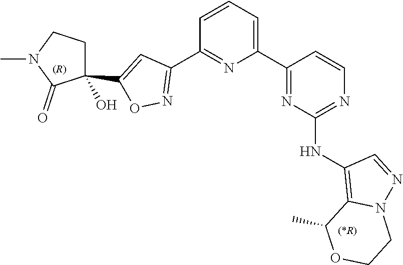

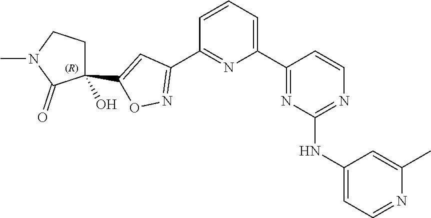

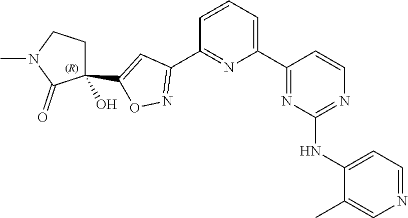

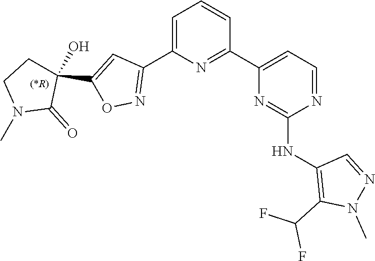 [SMILES: CN1CC[C@@](O)(c2cc(-c3cccc(-c4ccnc(Nc5cnn(C)c5C(F)F)n4)n3)no2)C1=O]

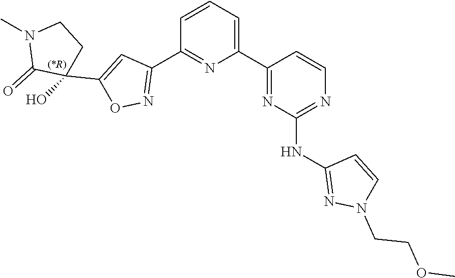

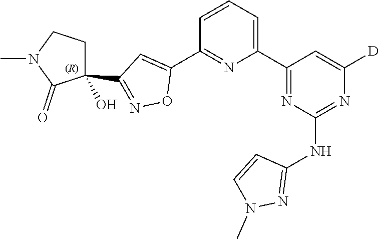

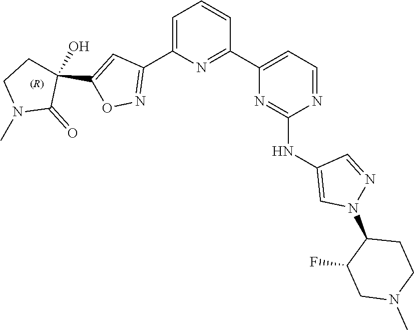

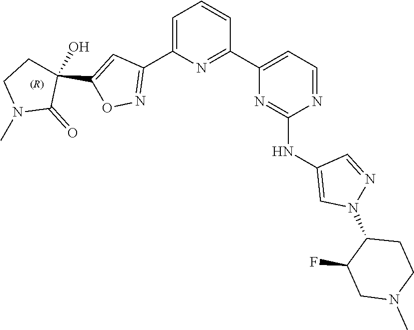

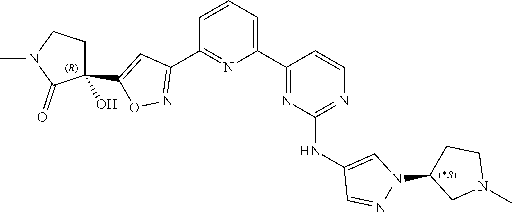

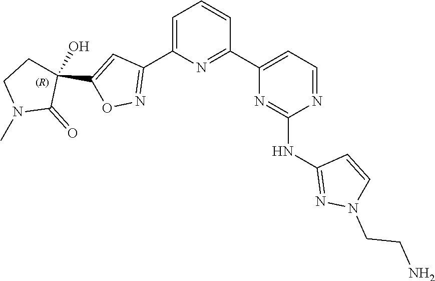

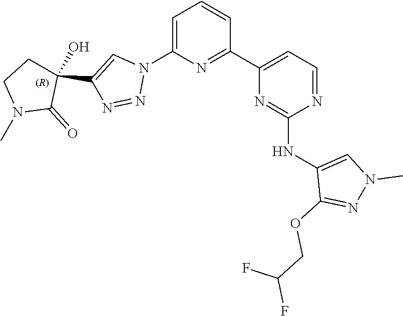

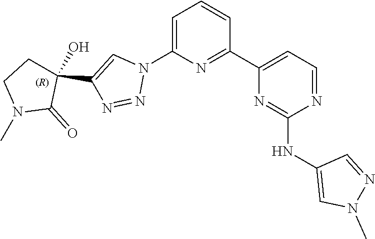

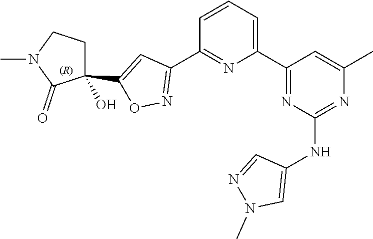

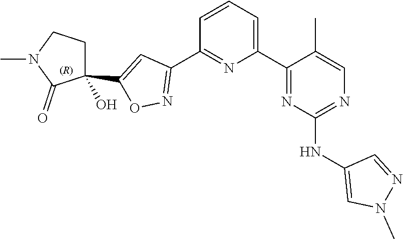

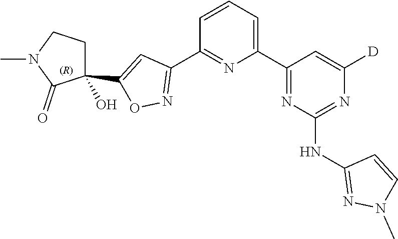

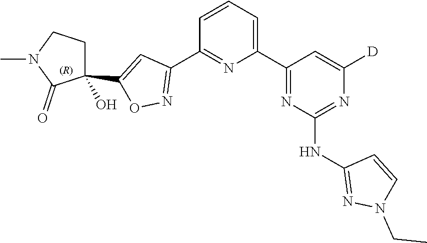

TABLE 2J

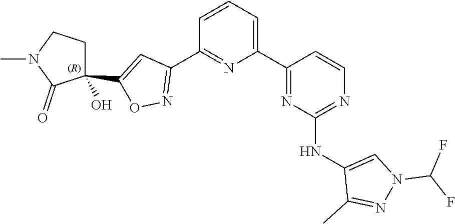

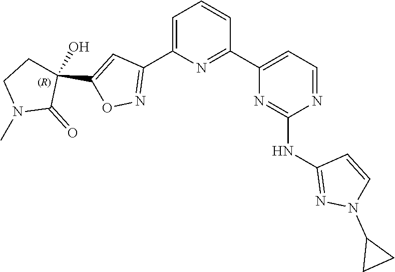

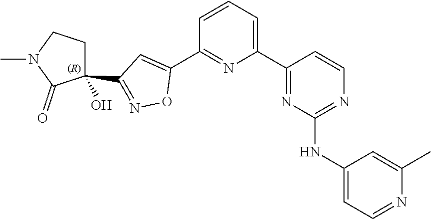

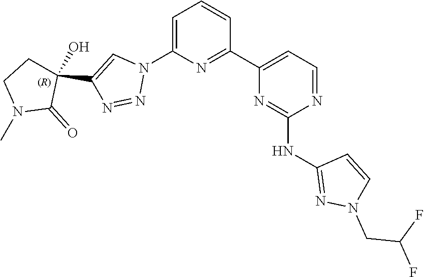

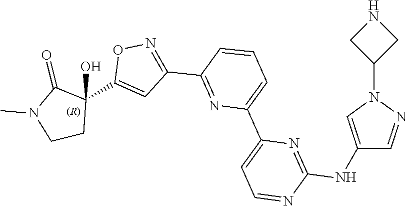

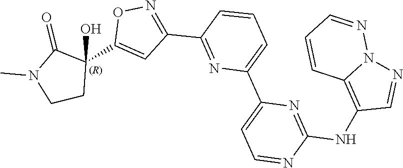

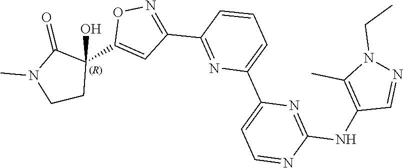

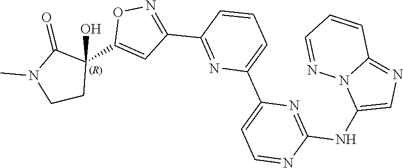

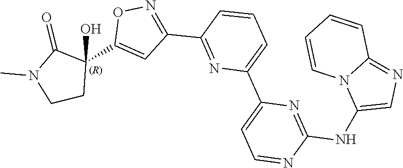

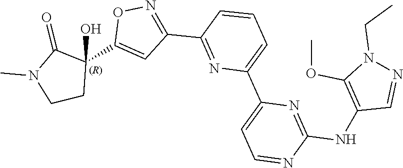

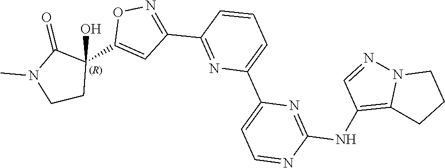

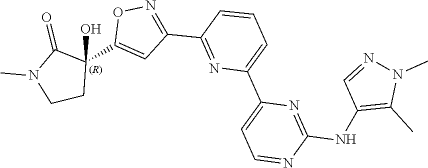

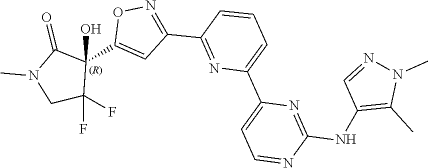

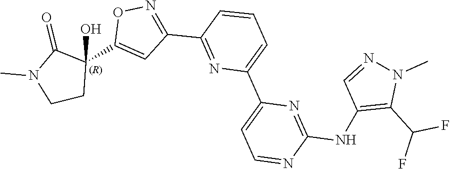

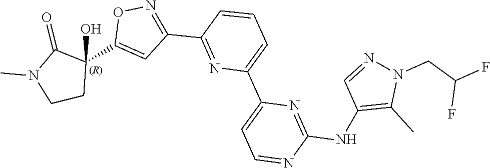

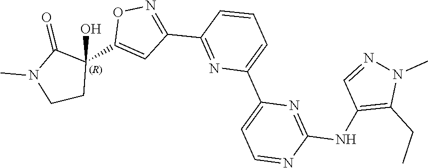

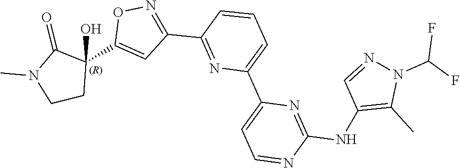

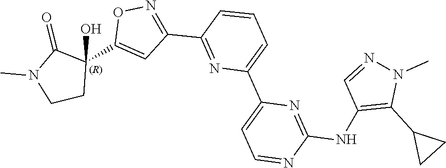

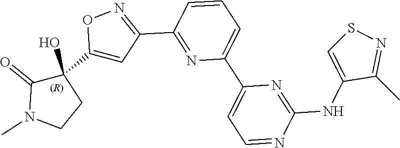

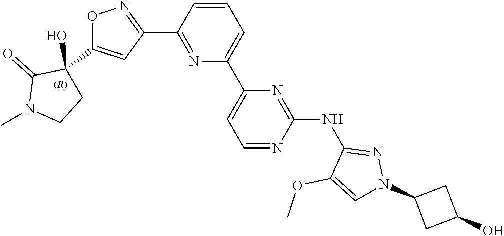

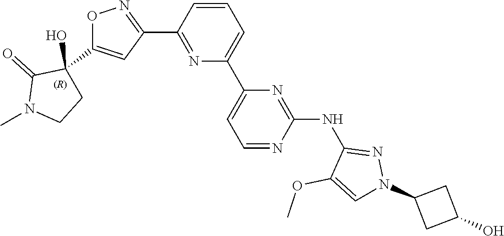

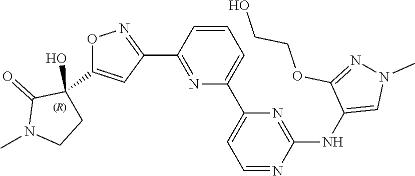

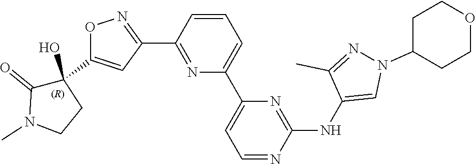

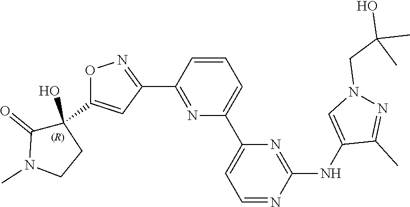

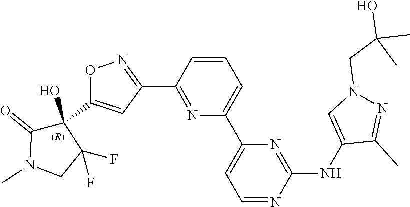

TABLE 2K

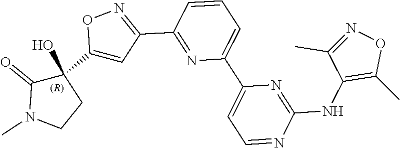

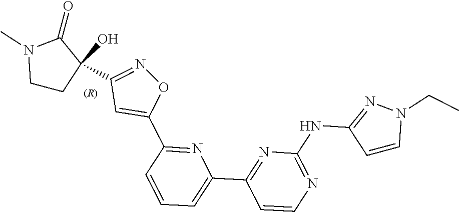

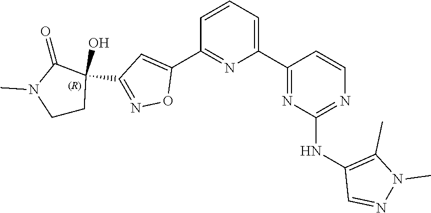

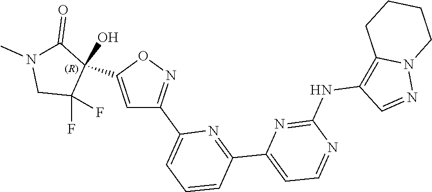

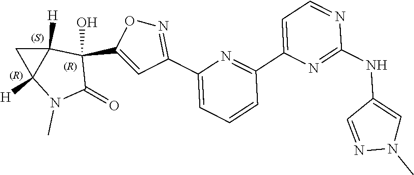

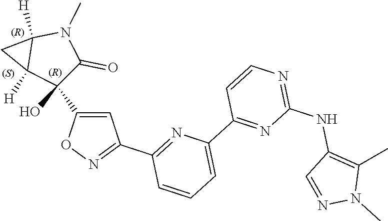

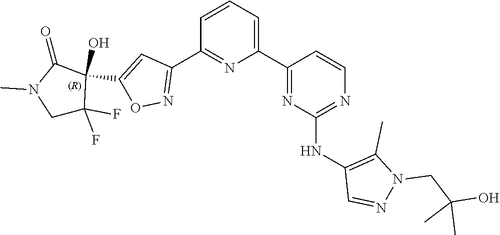

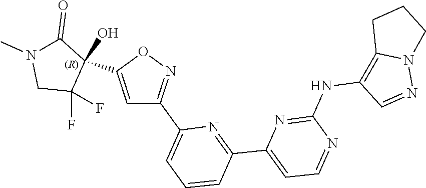

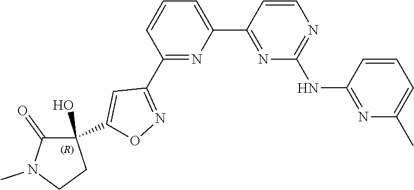

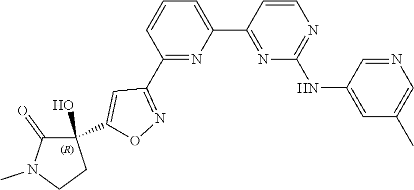

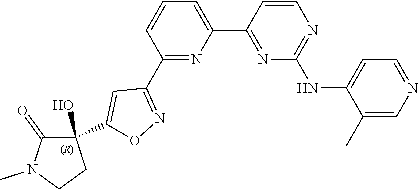

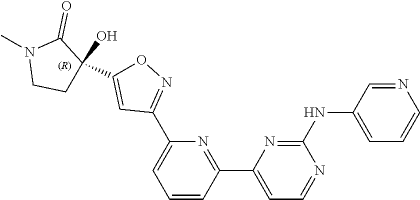

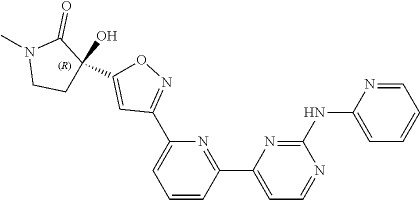

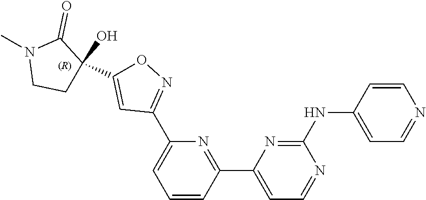

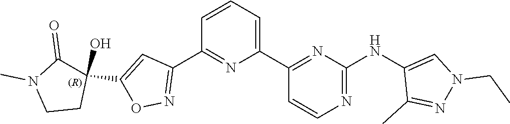

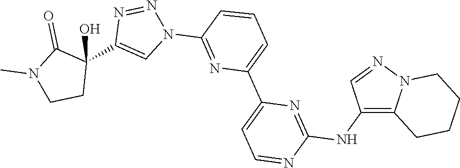 [SMILES: CN1CC[C@@](O)(c2cn(-c3cccc(-c4ccnc(Nc5cnn6c5CCCC6)n4)n3)nn2)C1=O]

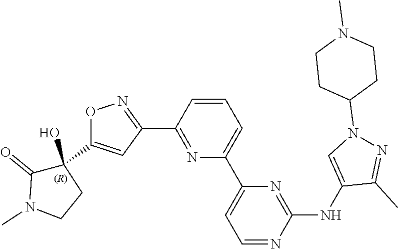

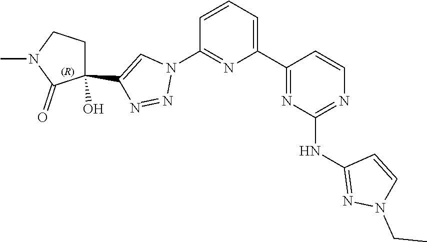

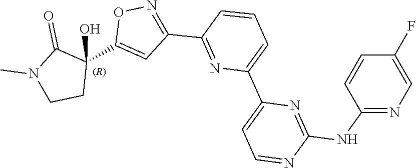

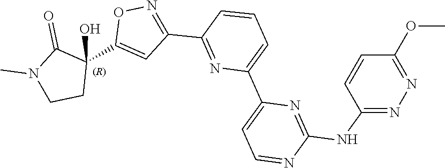

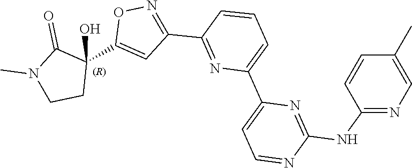

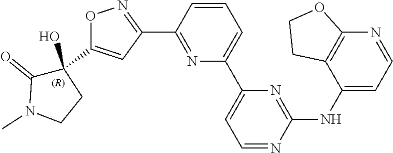

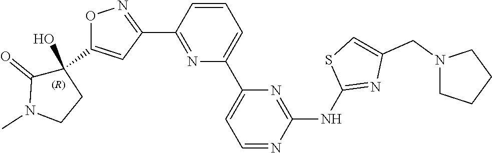

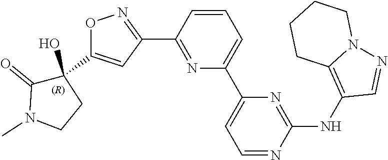

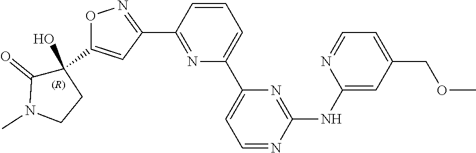

TABLE 2L

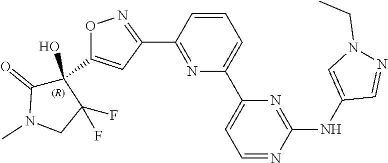

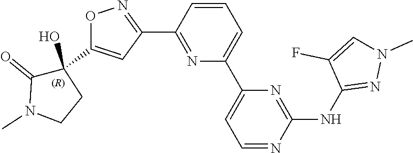

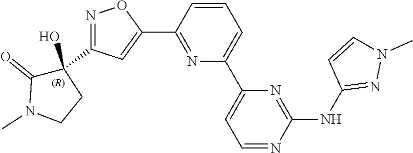

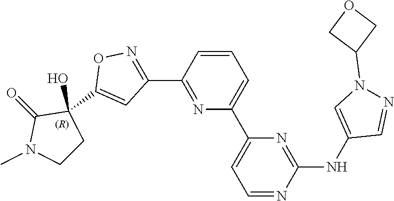

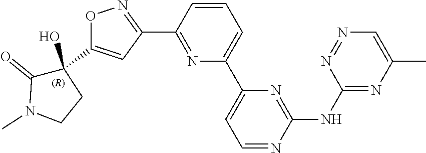

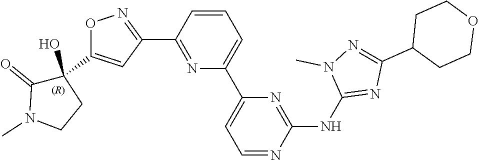

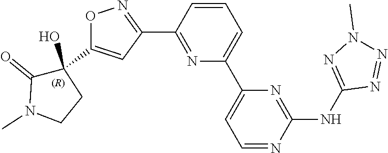

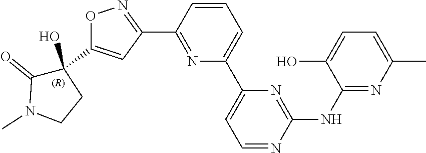

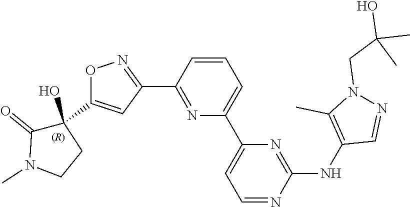

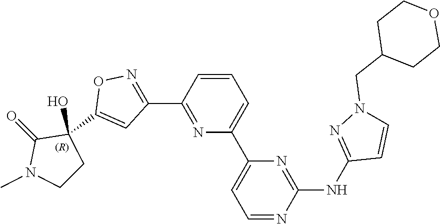

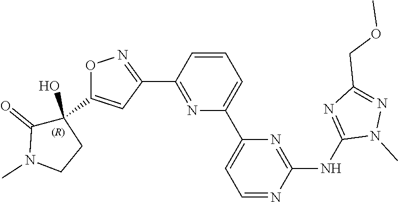

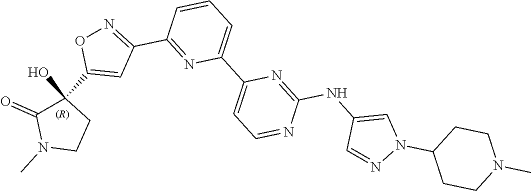

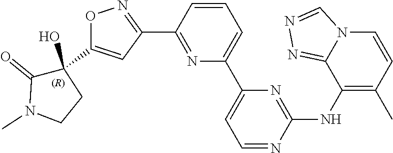

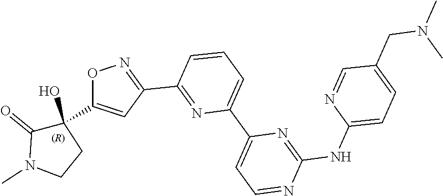

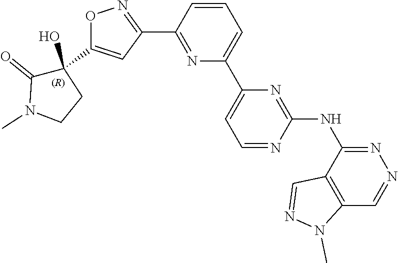

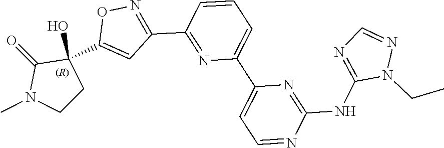

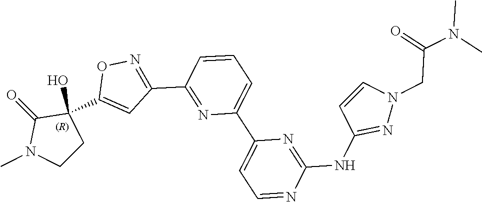

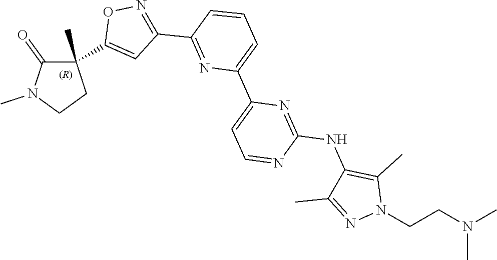

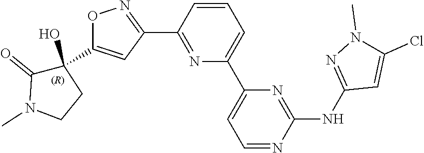

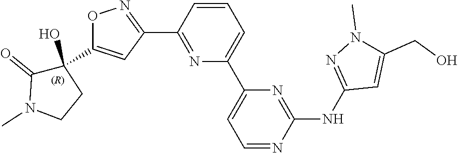

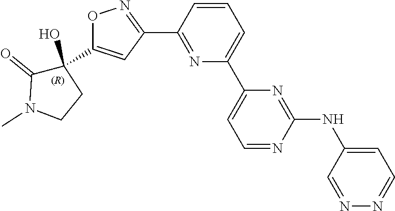

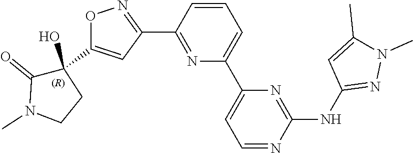

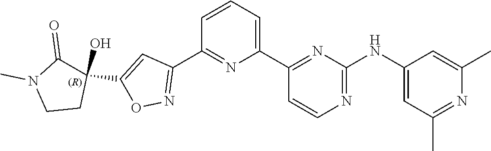

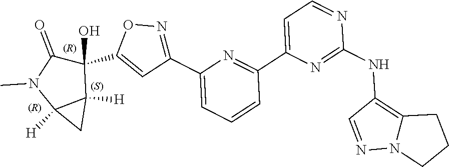

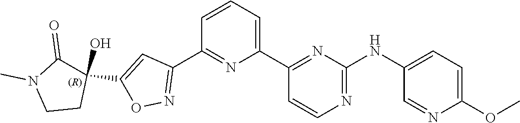

TABLE 2M

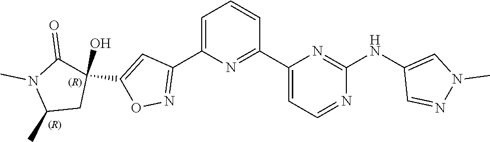

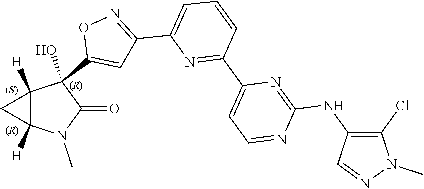

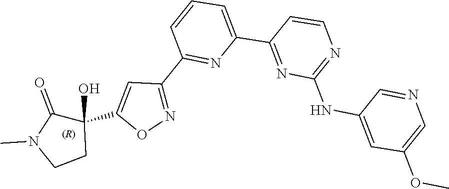

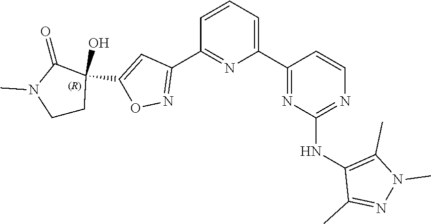

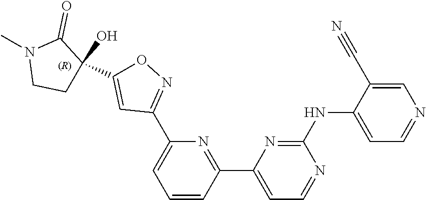

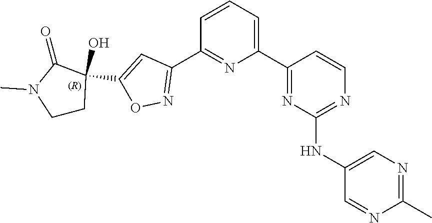

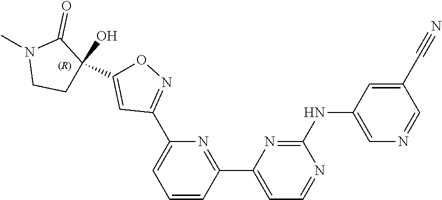

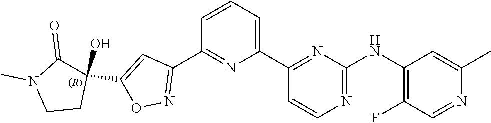

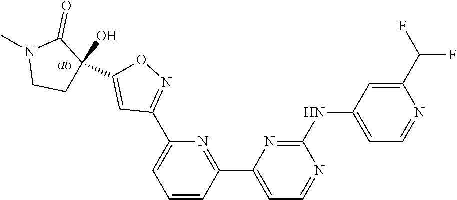

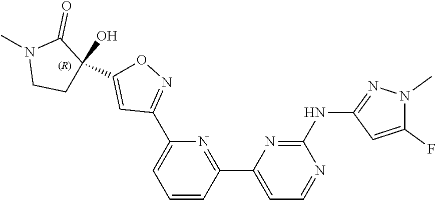

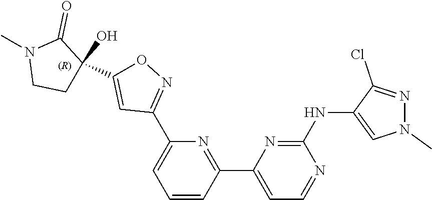

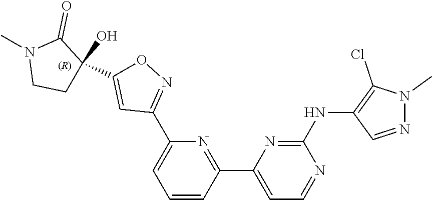

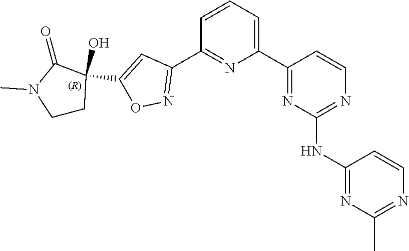

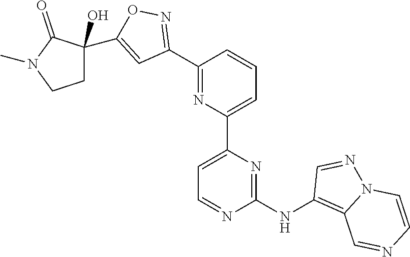 [SMILES: CN1CC[C@@](O)(c2cc(-c3cccc(-c4ccnc(Nc5cnn6ccncc56)n4)n3)no2)C1=O]

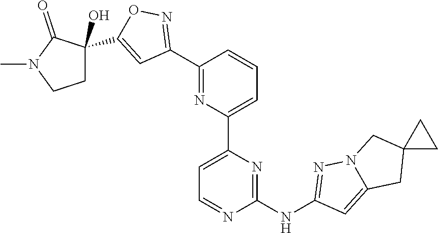 [SMILES: CN1CC[C@@](O)(c2cc(-c3cccc(-c4ccnc(Nc5cc6n(n5)CC5(CC5)C6)n4)n3)no2)C1=O]

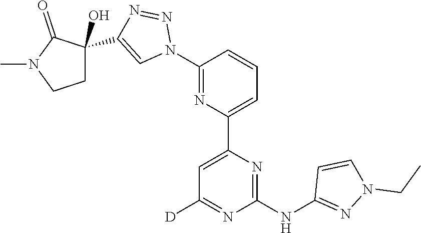 [SMILES: [2H]c1cc(-c2cccc(-n3cc([C@]4(O)CCN(C)C4=O)nn3)n2)nc(Nc2ccn(CC)n2)n1]

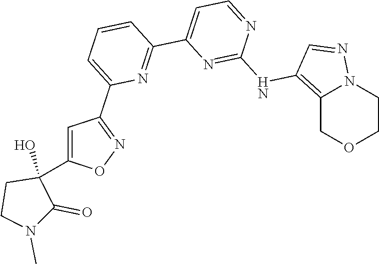 [SMILES: CN1CC[C@@](O)(c2cc(-c3cccc(-c4ccnc(Nc5cnn6c5COCC6)n4)n3)no2)C1=O]

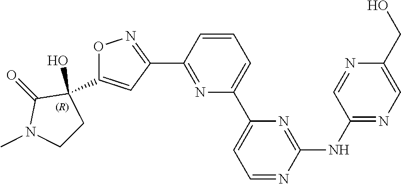

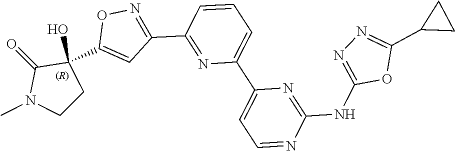

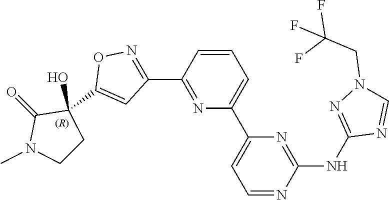

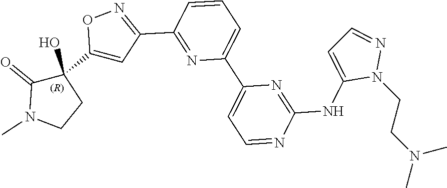

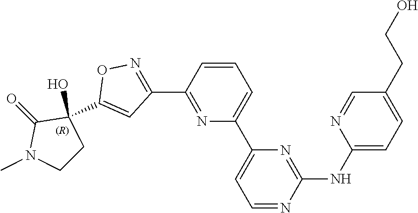

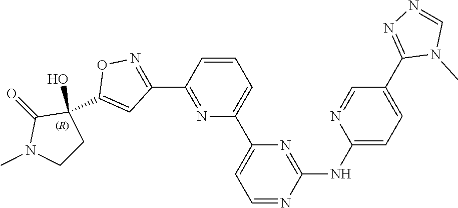

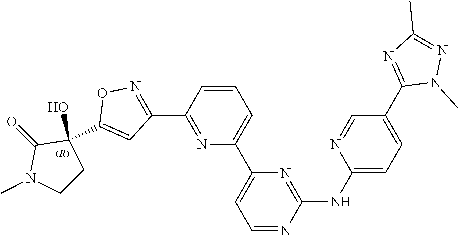

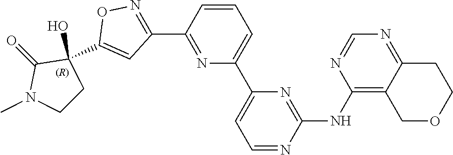

TABLE 2N

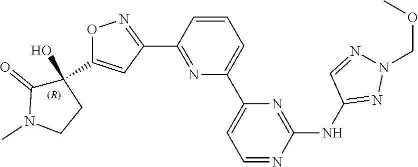

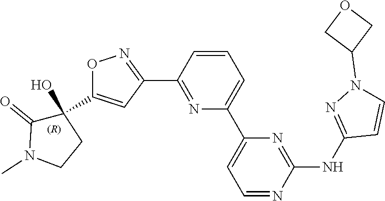

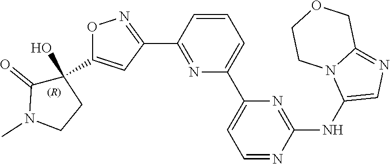

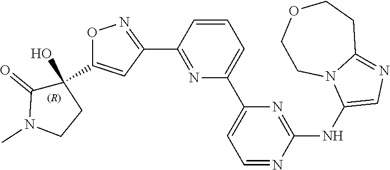

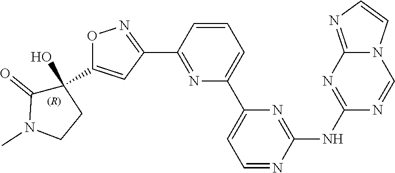

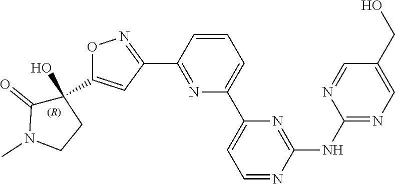

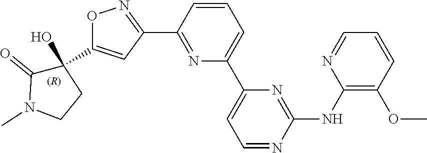

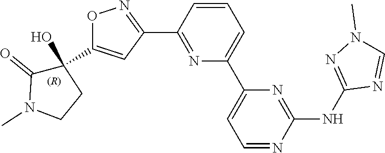

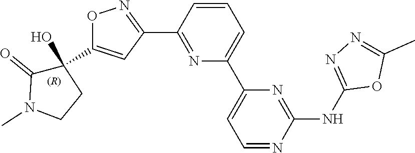

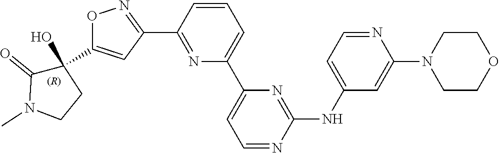

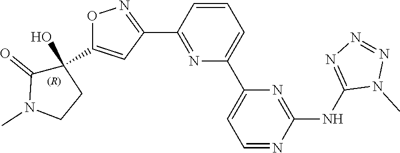

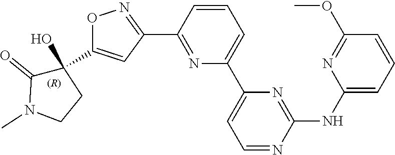

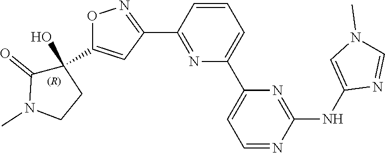

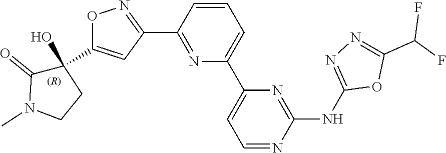

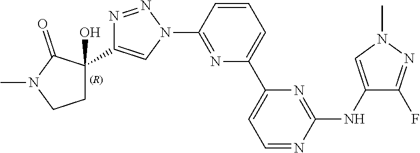

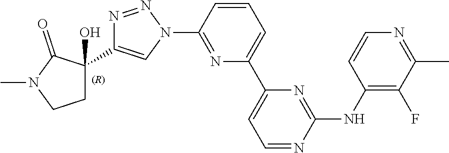

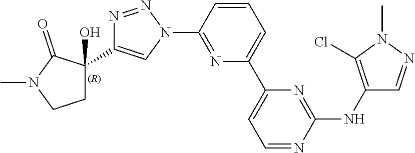

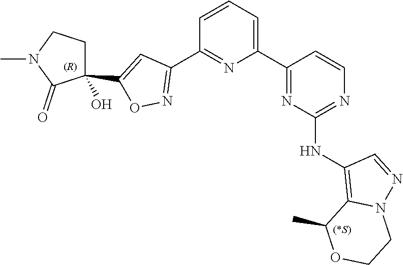

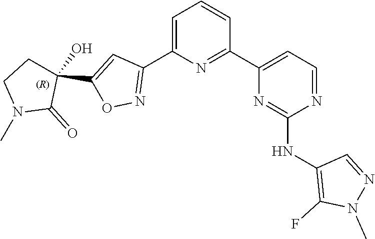

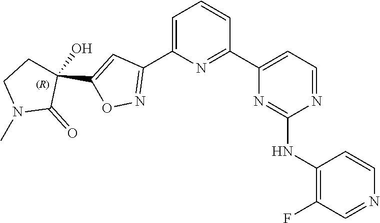

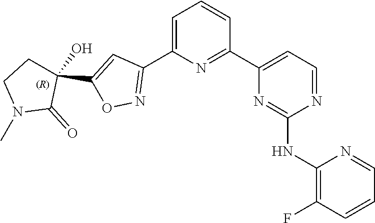

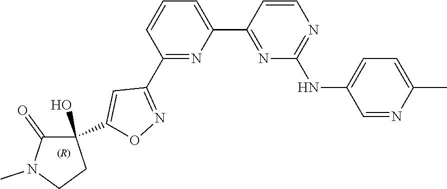

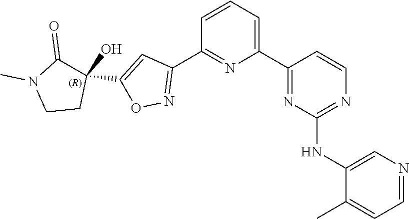

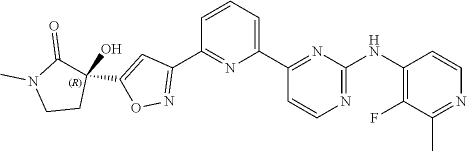

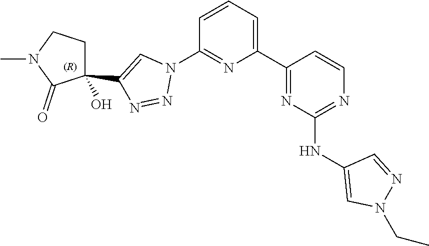

TABLE 20

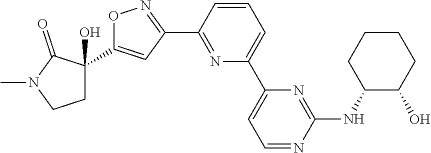 [SMILES: CN1CC[C@@](O)(c2cc(-c3cccc(-c4ccnc(N[C@@H]5CCCC[C@@H]5O)n4)n3)no2)C1=O]

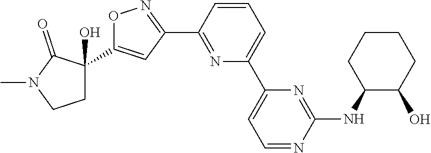 [SMILES: CN1CC[C@@](O)(c2cc(-c3cccc(-c4ccnc(N[C@H]5CCCC[C@H]5O)n4)n3)no2)C1=O]

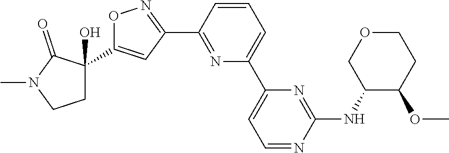

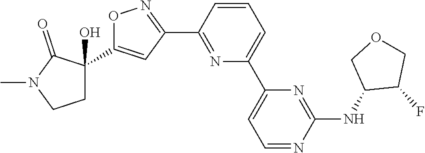 [SMILES: CN1CC[C@@](O)(c2cc(-c3cccc(-c4ccnc(N[C@@H]5COC[C@@H]5F)n4)n3)no2)C1=O]

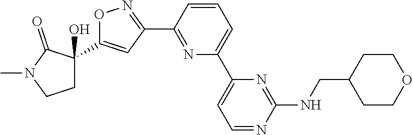 [SMILES: CN1CC[C@@](O)(c2cc(-c3cccc(-c4ccnc(NCC5CCOCC5)n4)n3)no2)C1=O]

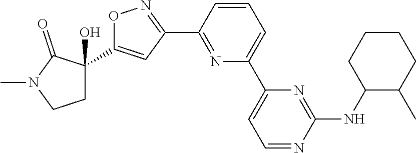

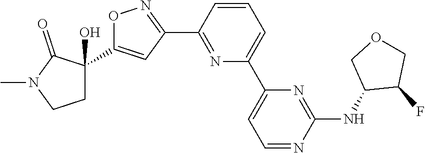 [SMILES: CN1CC[C@@](O)(c2cc(-c3cccc(-c4ccnc(N[C@@H]5COC[C@H]5F)n4)n3)no2)C1=O]

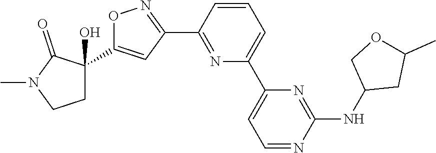

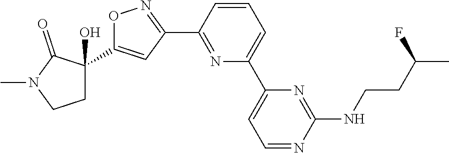 [SMILES: C[C@H](F)CCNc1nccc(-c2cccc(-c3cc([C@]4(O)CCN(C)C4=O)on3)n2)n1]

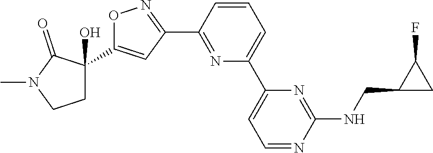 [SMILES: CN1CC[C@@](O)(c2cc(-c3cccc(-c4ccnc(NC[C@@H]5C[C@@H]5F)n4)n3)no2)C1=O]

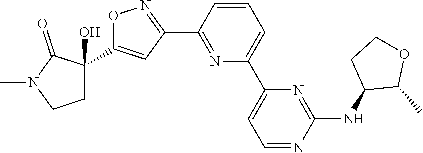

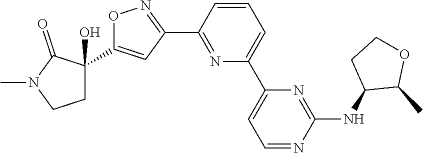

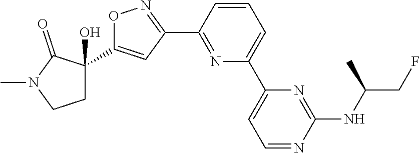 [SMILES: C[C@@H](CF)Nc1nccc(-c2cccc(-c3cc([C@]4(O)CCN(C)C4=O)on3)n2)n1]

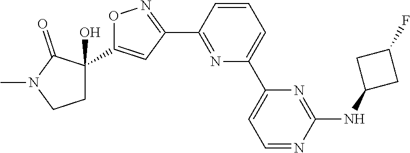 [SMILES: CN1CC[C@@](O)(c2cc(-c3cccc(-c4ccnc(N[C@H]5C[C@H](F)C5)n4)n3)no2)C1=O]

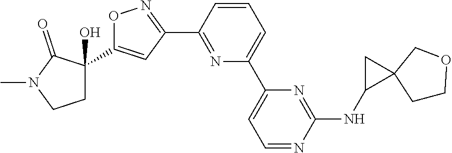 [SMILES: CN1CC[C@@](O)(c2cc(-c3cccc(-c4ccnc(NC5CC56CCOC6)n4)n3)no2)C1=O]

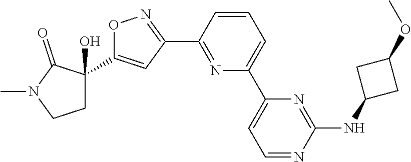 [SMILES: CO[C@H]1C[C@@H](Nc2nccc(-c3cccc(-c4cc([C@]5(O)CCN(C)C5=O)on4)n3)n2)C1]

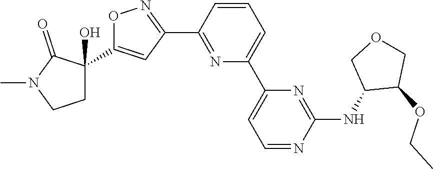 [SMILES: CCO[C@@H]1COC[C@H]1Nc1nccc(-c2cccc(-c3cc([C@]4(O)CCN(C)C4=O)on3)n2)n1]

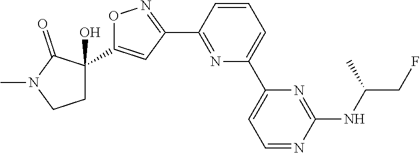 [SMILES: C[C@H](CF)Nc1nccc(-c2cccc(-c3cc([C@]4(O)CCN(C)C4=O)on3)n2)n1]

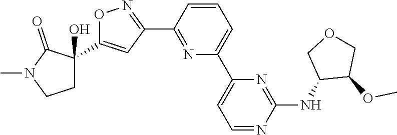

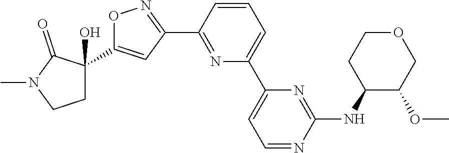

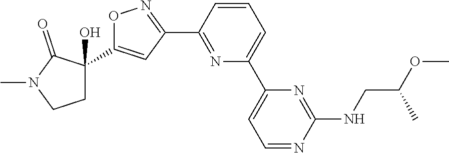

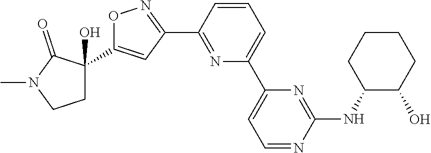 [SMILES: CN1CC[C@@](O)(c2cc(-c3cccc(-c4ccnc(N[C@@H]5CCCC[C@@H]5O)n4)n3)no2)C1=O]

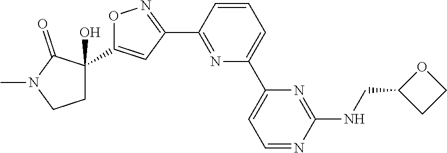 [SMILES: CN1CC[C@@](O)(c2cc(-c3cccc(-c4ccnc(NC[C@H]5CCO5)n4)n3)no2)C1=O]

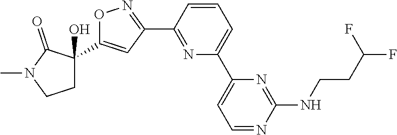 [SMILES: CN1CC[C@@](O)(c2cc(-c3cccc(-c4ccnc(NCCC(F)F)n4)n3)no2)C1=O]

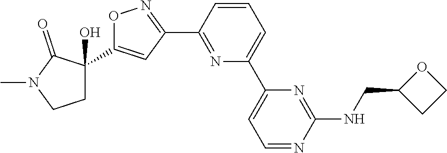 [SMILES: CN1CC[C@@](O)(c2cc(-c3cccc(-c4ccnc(NC[C@@H]5CCO5)n4)n3)no2)C1=O]

TABLE 2P

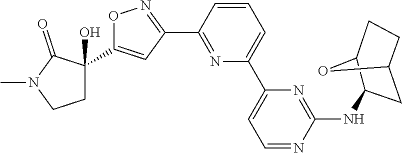 [SMILES: CN1CC[C@@](O)(c2cc(-c3cccc(-c4ccnc(N[C@@H]5CC6CCC5O6)n4)n3)no2)C1=O]

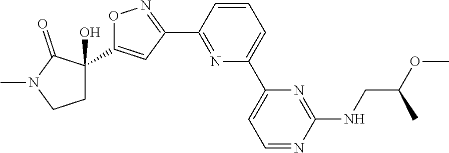

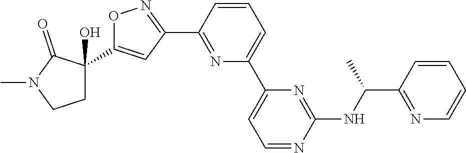 [SMILES: C[C@@H](Nc1nccc(-c2cccc(-c3cc([C@]4(O)CCN(C)C4=O)on3)n2)n1)c1ccccn1]

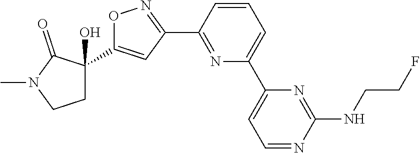 [SMILES: CN1CC[C@@](O)(c2cc(-c3cccc(-c4ccnc(NCCF)n4)n3)no2)C1=O]

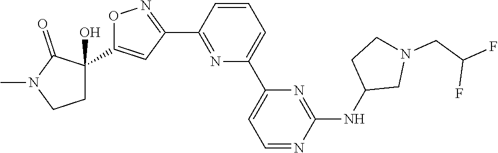 [SMILES: CN1CC[C@@](O)(c2cc(-c3cccc(-c4ccnc(NC5CCN(CC(F)F)C5)n4)n3)no2)C1=O]

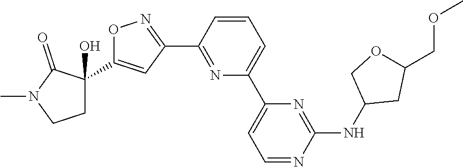

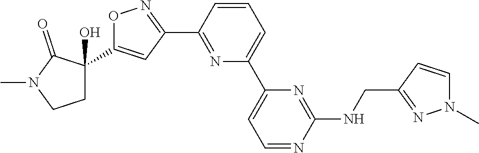

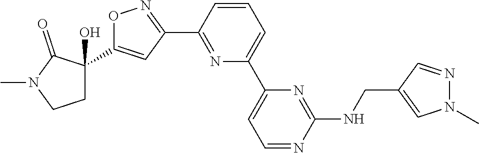

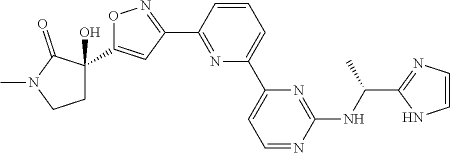 [SMILES: C[C@@H](Nc1nccc(-c2cccc(-c3cc([C@]4(O)CCN(C)C4=O)on3)n2)n1)c1ncc[nH]1]

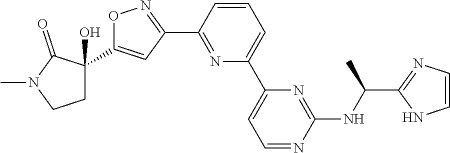 [SMILES: C[C@H](Nc1nccc(-c2cccc(-c3cc([C@]4(O)CCN(C)C4=O)on3)n2)n1)c1ncc[nH]1]

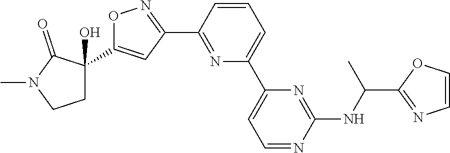 [SMILES: CC(Nc1nccc(-c2cccc(-c3cc([C@]4(O)CCN(C)C4=O)on3)n2)n1)c1ncco1]

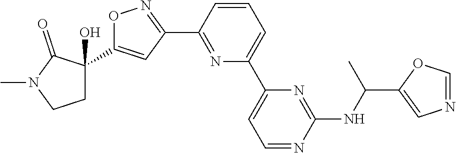 [SMILES: CC(Nc1nccc(-c2cccc(-c3cc([C@]4(O)CCN(C)C4=O)on3)n2)n1)c1cnco1]

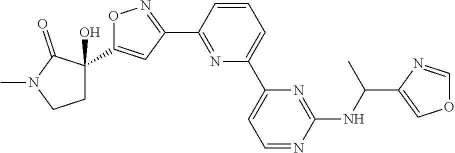 [SMILES: CC(Nc1nccc(-c2cccc(-c3cc([C@]4(O)CCN(C)C4=O)on3)n2)n1)c1cocn1]

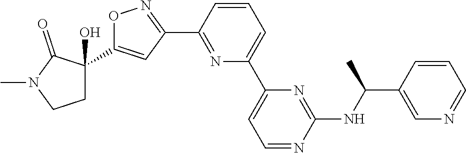 [SMILES: C[C@H](Nc1nccc(-c2cccc(-c3cc([C@]4(O)CCN(C)C4=O)on3)n2)n1)c1cccnc1]

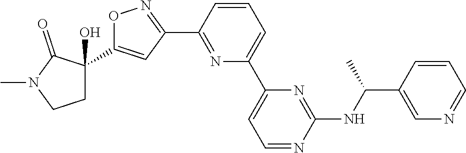 [SMILES: C[C@@H](Nc1nccc(-c2cccc(-c3cc([C@]4(O)CCN(C)C4=O)on3)n2)n1)c1cccnc1]

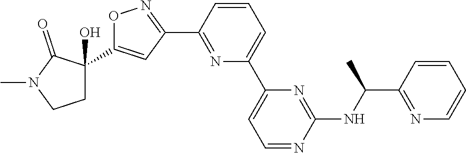 [SMILES: C[C@H](Nc1nccc(-c2cccc(-c3cc([C@]4(O)CCN(C)C4=O)on3)n2)n1)c1ccccn1]

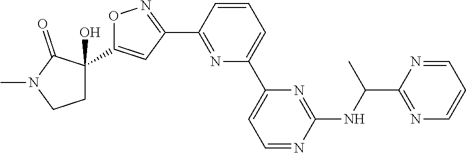 [SMILES: CC(Nc1nccc(-c2cccc(-c3cc([C@]4(O)CCN(C)C4=O)on3)n2)n1)c1ncccn1]

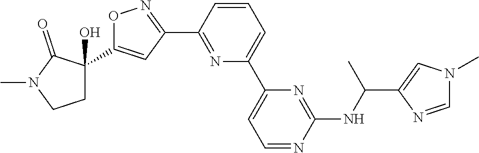

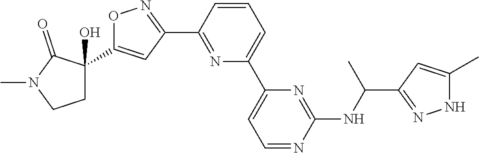

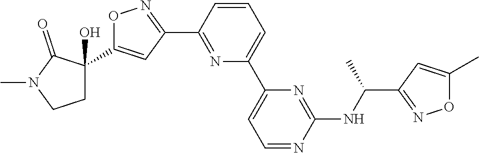

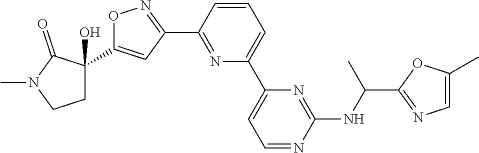

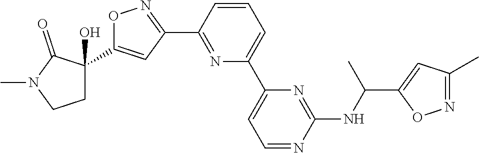

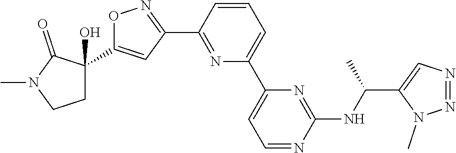 [SMILES: C[C@@H](Nc1nccc(-c2cccc(-c3cc([C@]4(O)CCN(C)C4=O)on3)n2)n1)c1cnnn1C]

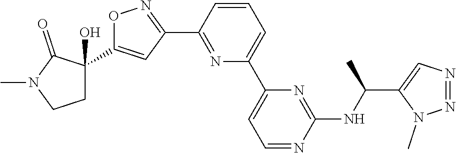 [SMILES: C[C@H](Nc1nccc(-c2cccc(-c3cc([C@]4(O)CCN(C)C4=O)on3)n2)n1)c1cnnn1C]

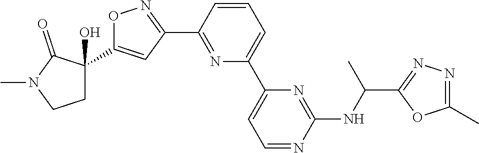

TABLE 2Q

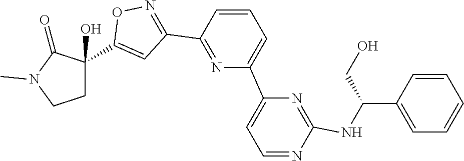 [SMILES: CN1CC[C@@](O)(c2cc(-c3cccc(-c4ccnc(N[C@H](CO)c5ccccc5)n4)n3)no2)C1=O]

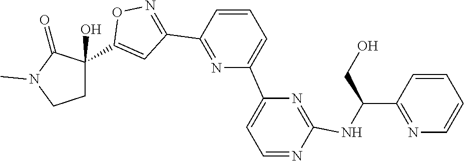 [SMILES: CN1CC[C@@](O)(c2cc(-c3cccc(-c4ccnc(N[C@@H](CO)c5ccccn5)n4)n3)no2)C1=O]

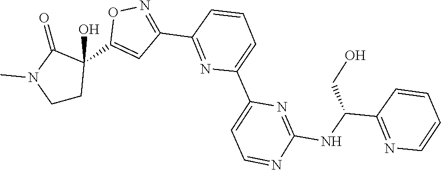 [SMILES: CN1CC[C@@](O)(c2cc(-c3cccc(-c4ccnc(N[C@H](CO)c5ccccn5)n4)n3)no2)C1=O]

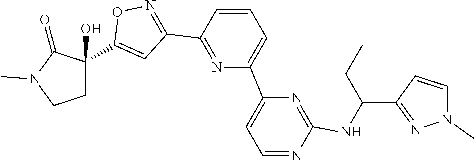

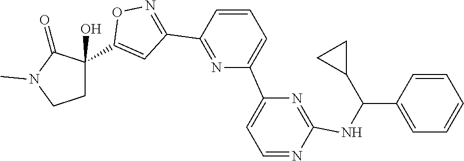 [SMILES: CN1CC[C@@](O)(c2cc(-c3cccc(-c4ccnc(NC(c5ccccc5)C5CC5)n4)n3)no2)C1=O]

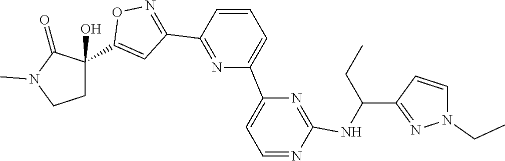 [SMILES: CCC(Nc1nccc(-c2cccc(-c3cc([C@]4(O)CCN(C)C4=O)on3)n2)n1)c1ccn(CC)n1]

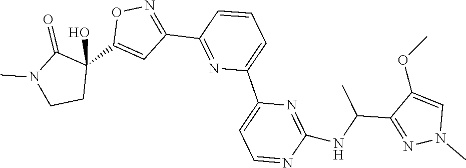

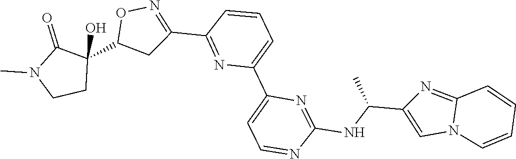 [SMILES: C[C@@H](Nc1nccc(-c2cccc(C3=NOC([C@]4(O)CCN(C)C4=O)C3)n2)n1)c1cn2ccccc2n1]

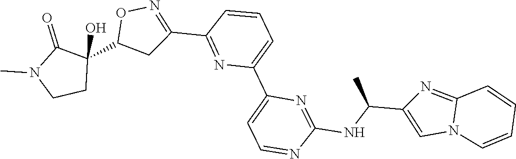 [SMILES: C[C@H](Nc1nccc(-c2cccc(C3=NOC([C@]4(O)CCN(C)C4=O)C3)n2)n1)c1cn2ccccc2n1]

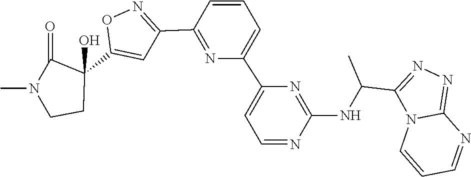 [SMILES: CC(Nc1nccc(-c2cccc(-c3cc([C@]4(O)CCN(C)C4=O)on3)n2)n1)c1nnc2ncccn12]

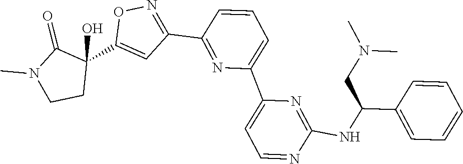 [SMILES: CN(C)C[C@H](Nc1nccc(-c2cccc(-c3cc([C@]4(O)CCN(C)C4=O)on3)n2)n1)c1ccccc1]

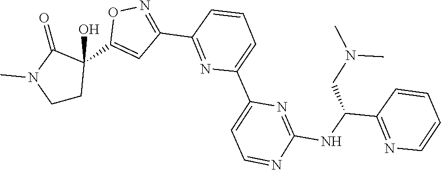 [SMILES: CN(C)C[C@@H](Nc1nccc(-c2cccc(-c3cc([C@]4(O)CCN(C)C4=O)on3)n2)n1)c1ccccn1]

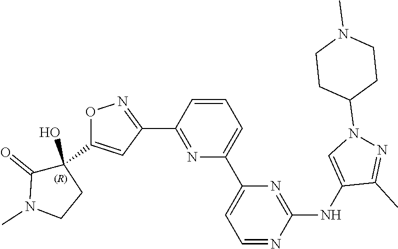

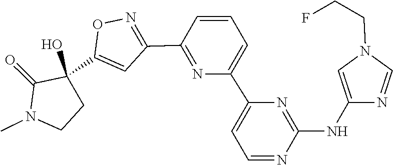 [SMILES: CN1CC[C@@](O)(c2cc(-c3cccc(-c4ccnc(Nc5cn(CCF)cn5)n4)n3)no2)C1=O]

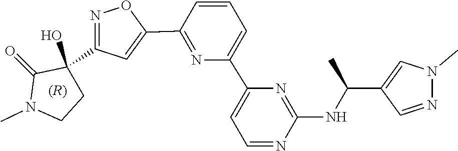

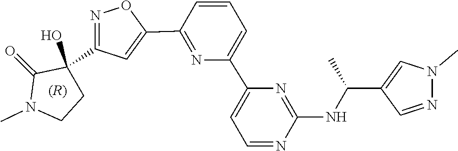

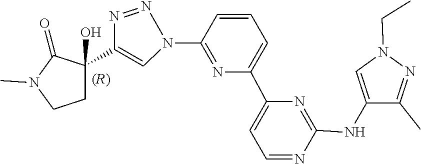

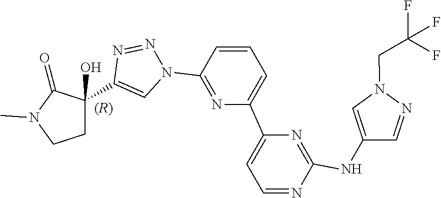

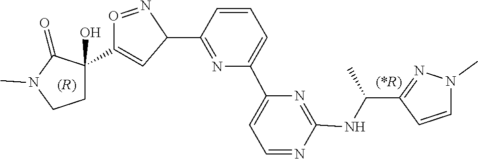

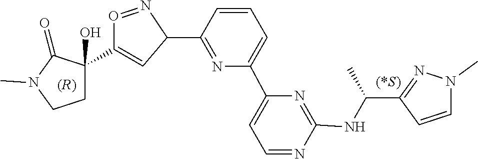

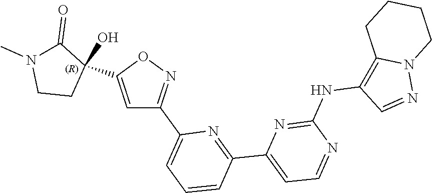

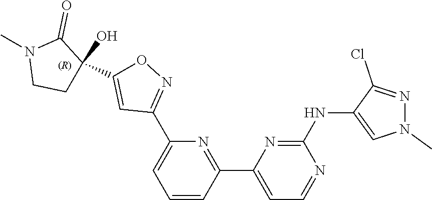

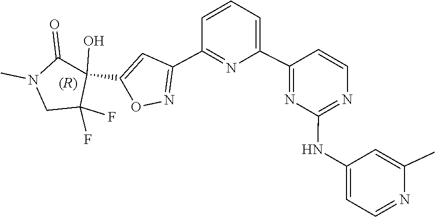

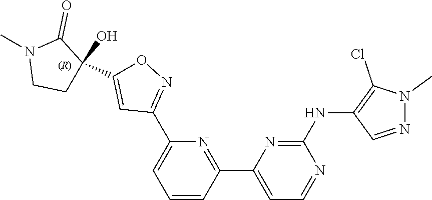

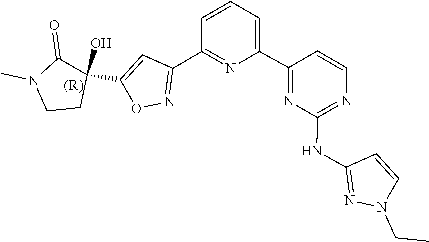

TABLE 2R

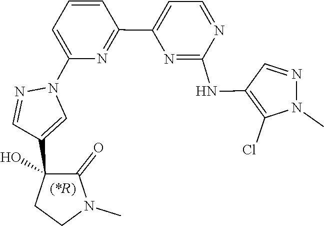

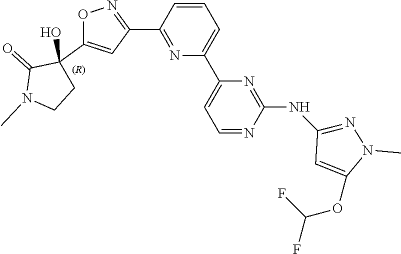

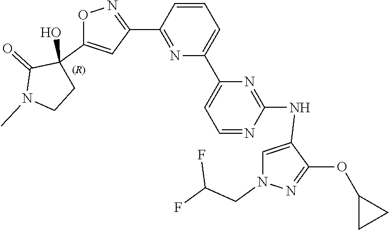

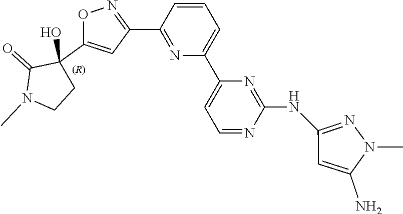

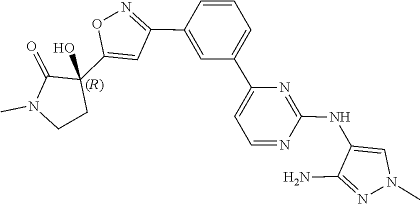

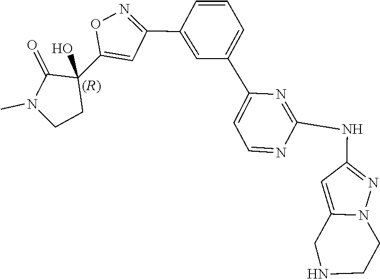

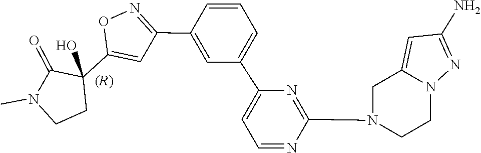

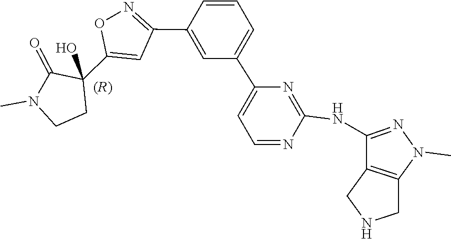

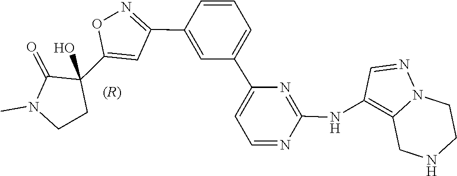

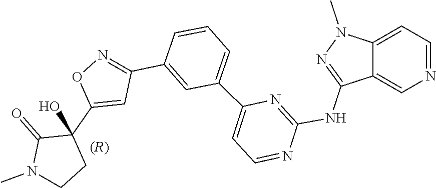

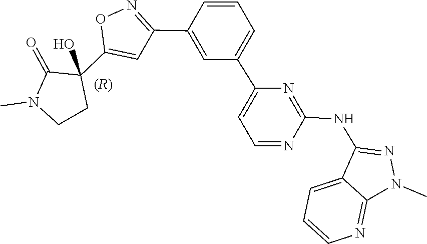

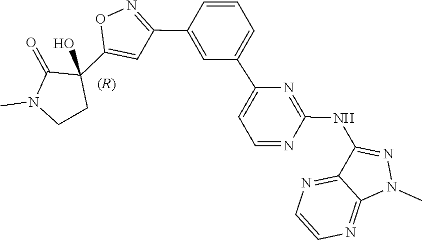

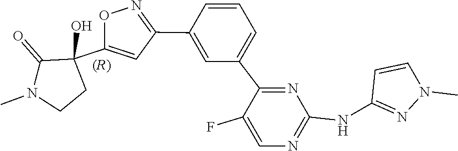

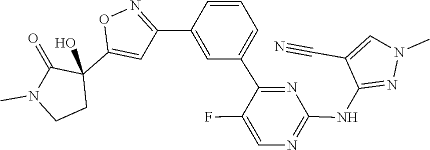

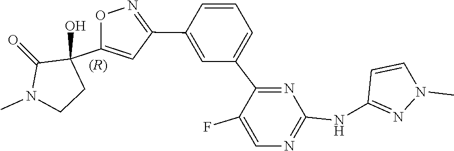

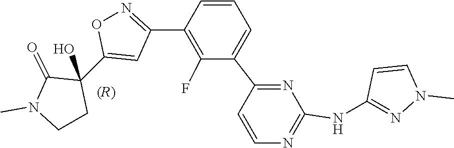

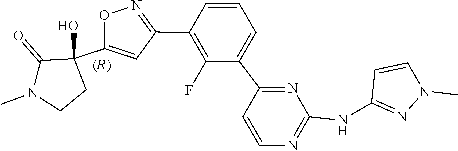

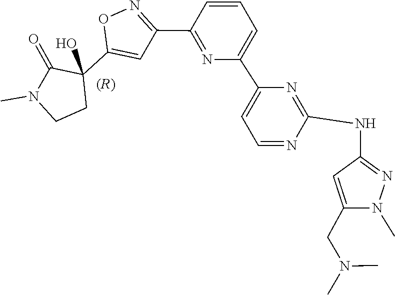

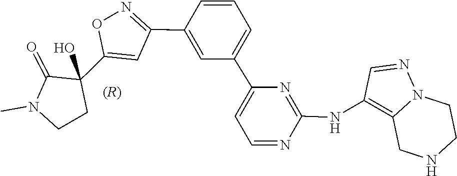

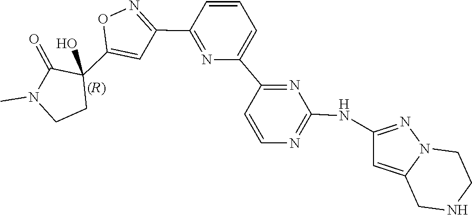

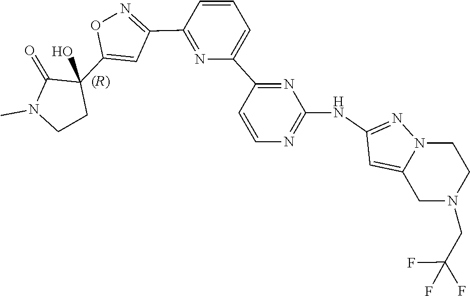

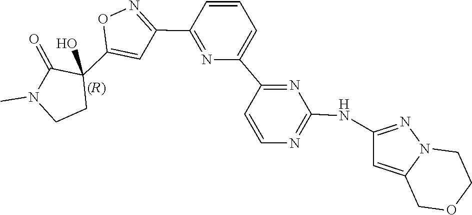

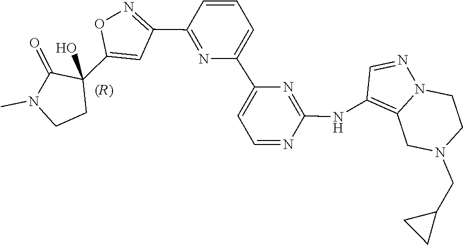

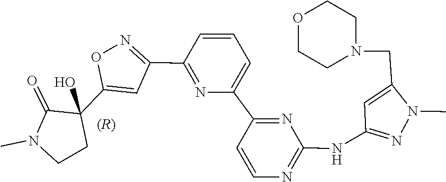

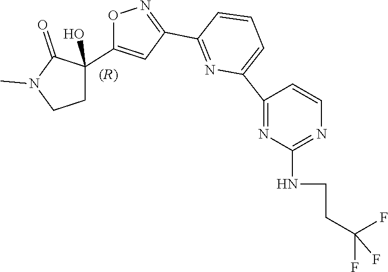

TABLE 2S

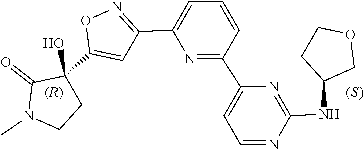

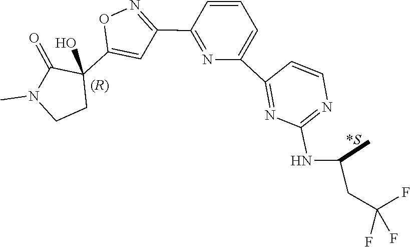

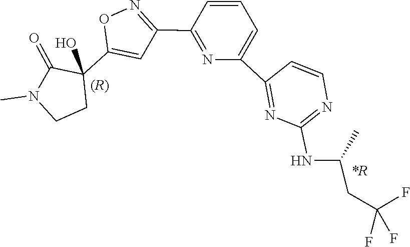

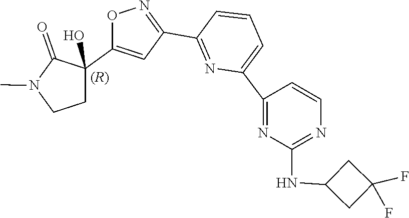

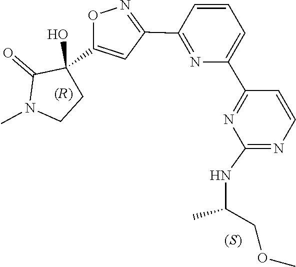

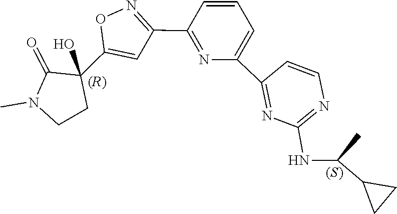

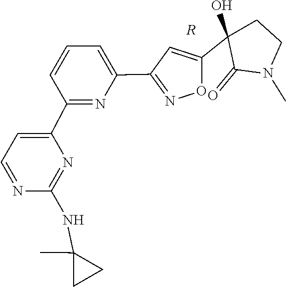

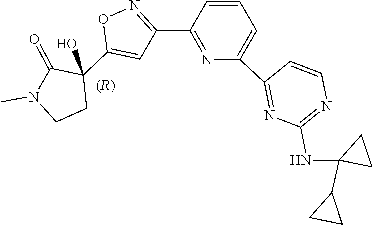

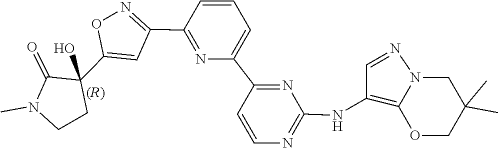

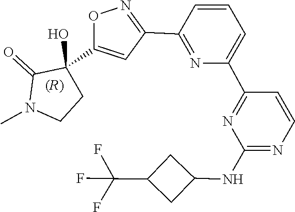

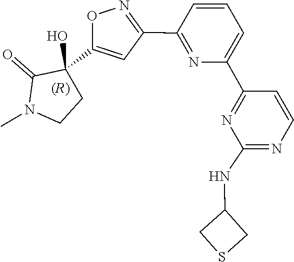

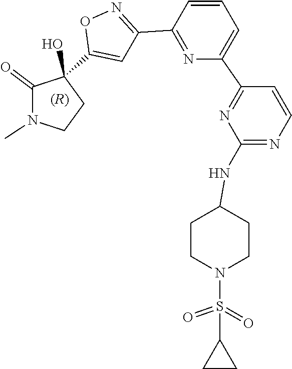

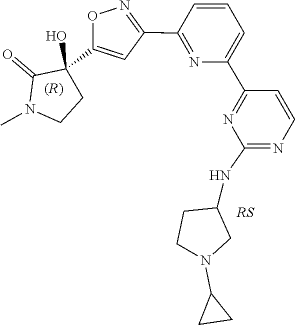

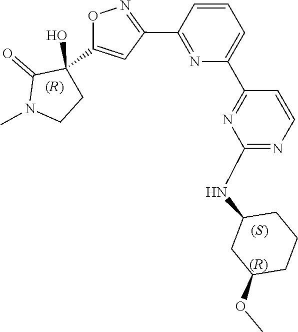

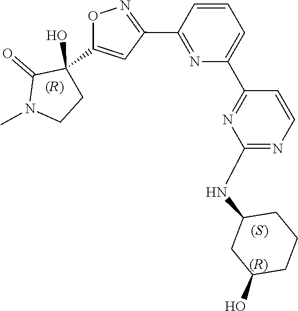

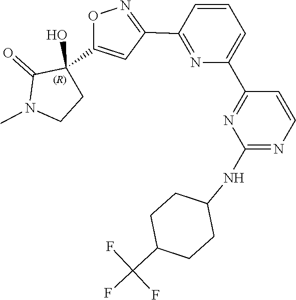

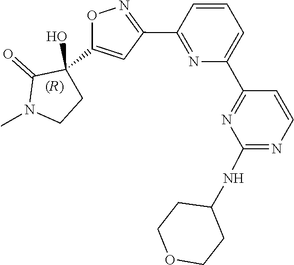

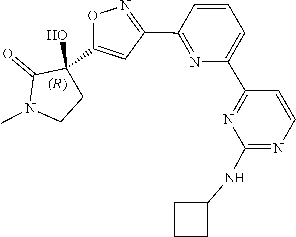

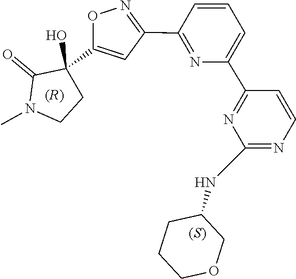

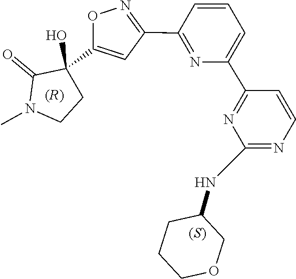

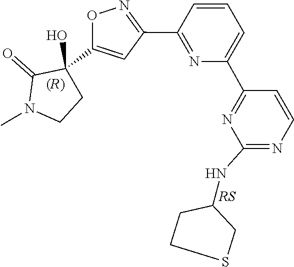

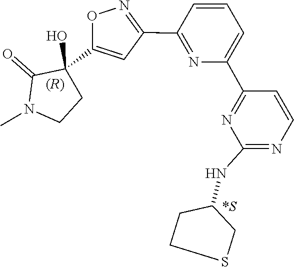

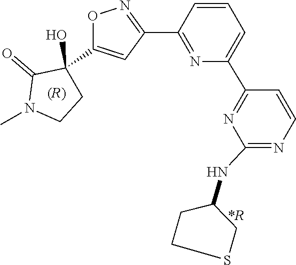

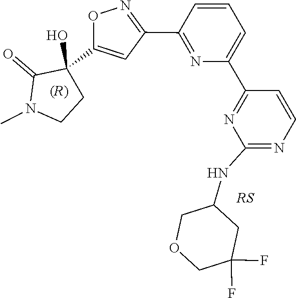

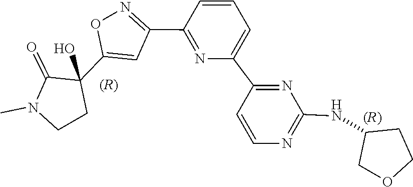

TABLE 2T

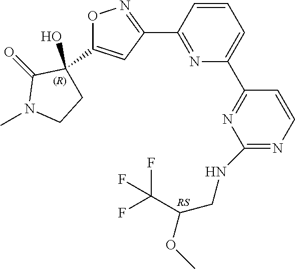

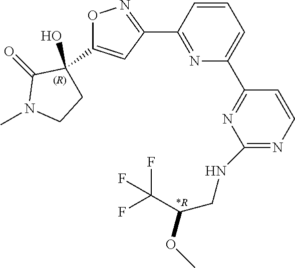

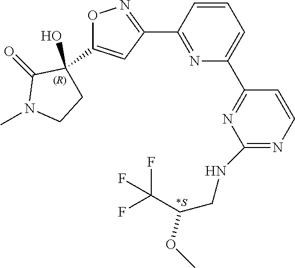

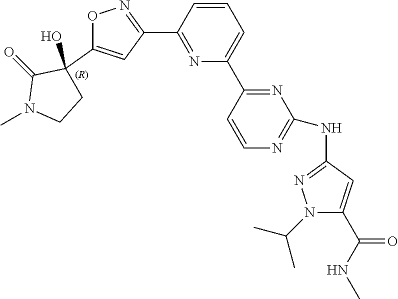

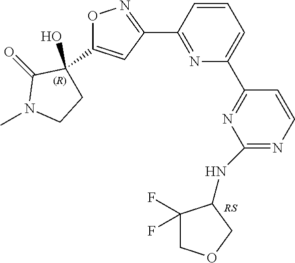

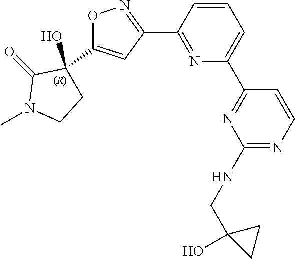

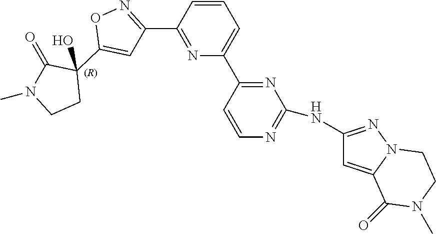

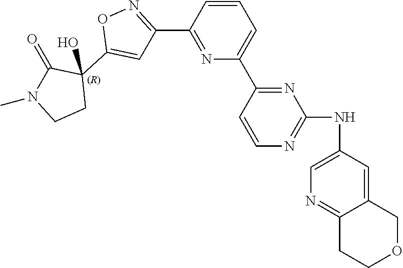

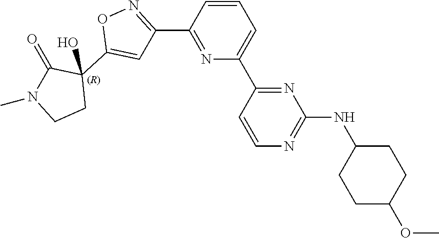

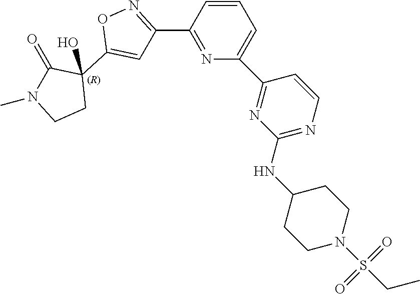

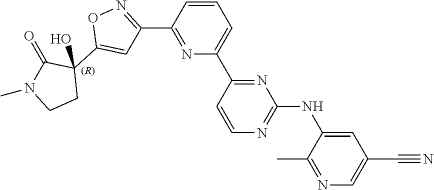

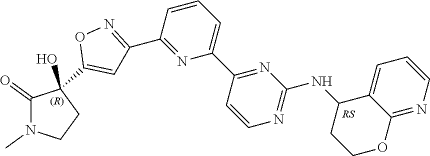

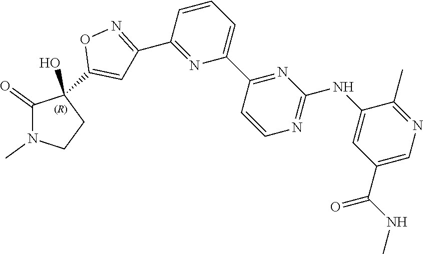

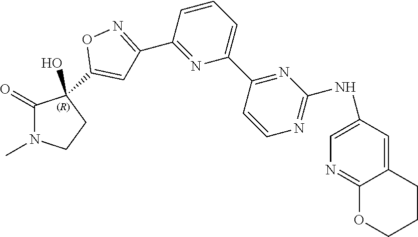

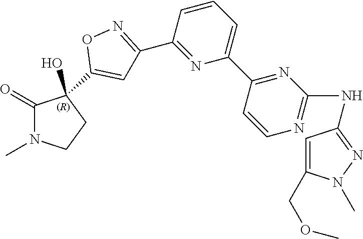

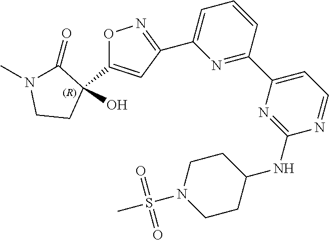

In some embodiments, disclosed herein is a compound of Formula I′ or Formula I or a pharmaceutically acceptable salt thereof, selected from the group consisting of:

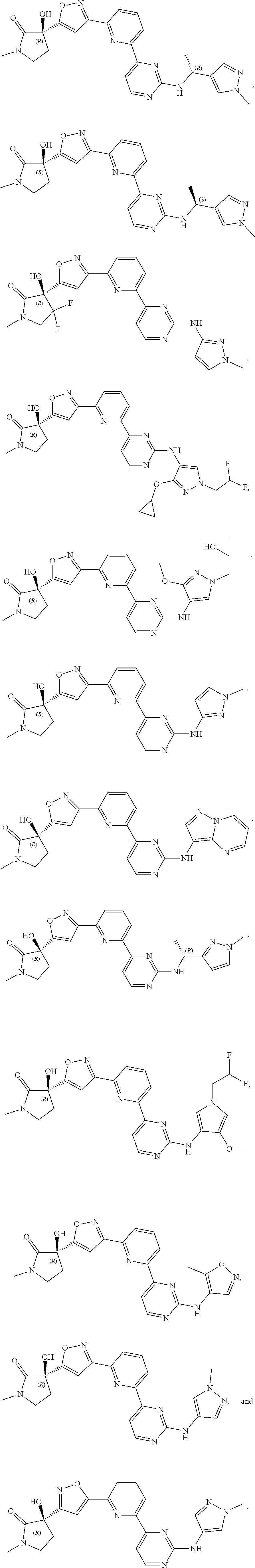

In some embodiments, disclosed herein is a compound of Formula I′ or Formula I or a pharmaceutically acceptable salt thereof, selected from the group consisting of:

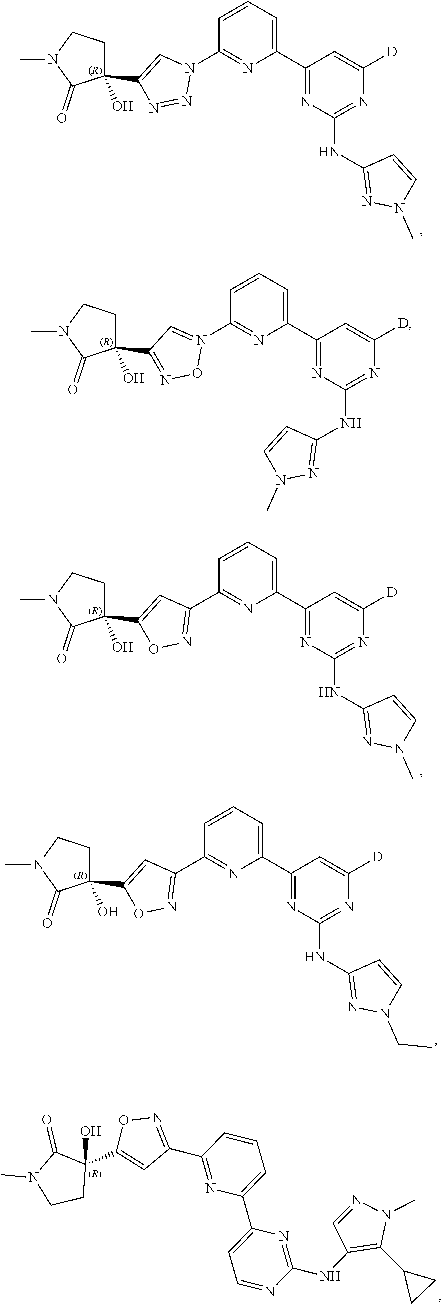

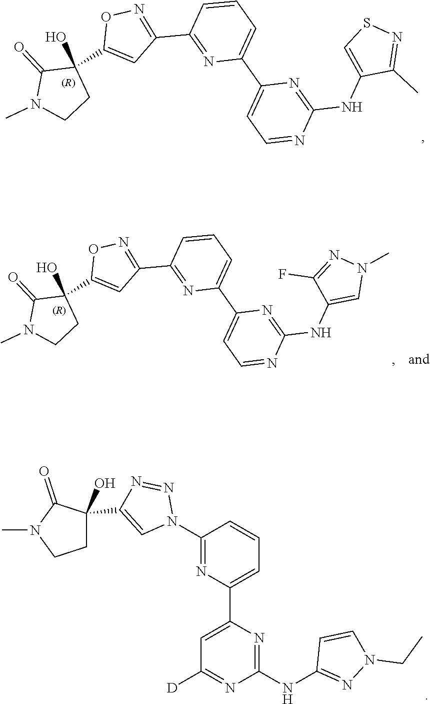

In some embodiments, disclosed herein is a compound of Formula I′ or Formula I having the following structure:

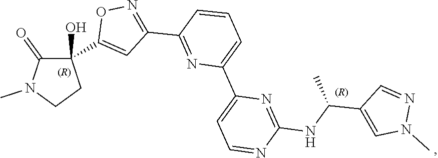

or a pharmaceutically acceptable salt thereof.

In some embodiments, disclosed herein is a compound of Formula I′ or Formula I having the following structure:

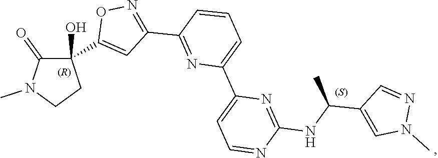

or a pharmaceutically acceptable salt thereof.

In some embodiments, disclosed herein is a compound of Formula I′ or Formula I having the following structure:

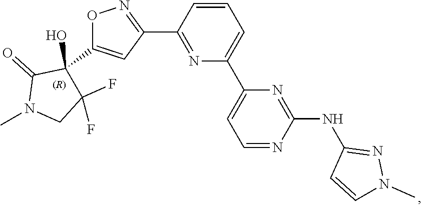

or a pharmaceutically acceptable salt thereof.

In some embodiments, disclosed herein is a compound of Formula I′ or Formula I having the following structure:

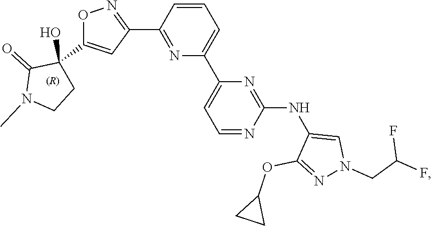

or a pharmaceutically acceptable salt thereof.

In some embodiments, disclosed herein is a compound of Formula I′ or Formula I having the following structure:

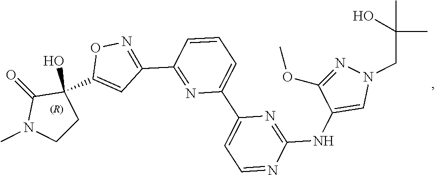

or a pharmaceutically acceptable salt thereof.

In some embodiments, disclosed herein is a compound of Formula I′ or Formula I having the following structure:

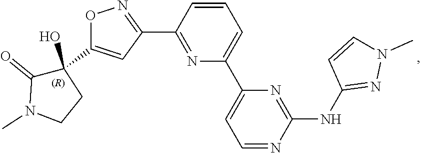

or a pharmaceutically acceptable salt thereof.

In some embodiments, disclosed herein is a compound of Formula I′ or Formula I having the following structure:

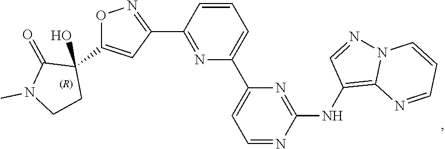

or a pharmaceutically acceptable salt thereof.

In some embodiments, disclosed herein is a compound of Formula I′ or Formula I having the following structure:

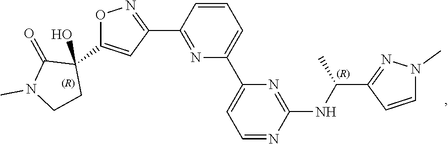

or a pharmaceutically acceptable salt thereof.

In some embodiments, disclosed herein is a compound of Formula I′ or Formula I having the following structure:

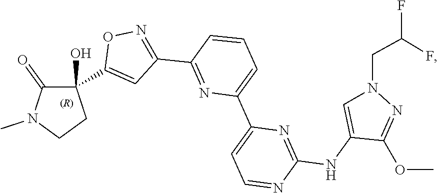

or a pharmaceutically acceptable salt thereof.

In some embodiments, disclosed herein is a compound of Formula I′ or Formula I having the following structure:

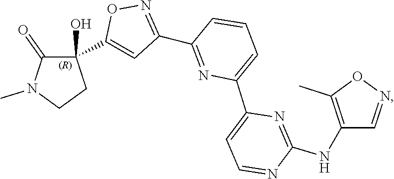

or a pharmaceutically acceptable salt thereof.

In some embodiments, disclosed herein is a compound of Formula I′ or Formula I having the following structure:

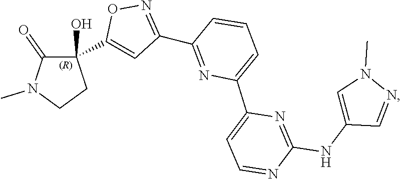

or a pharmaceutically acceptable salt thereof.

In some embodiments, disclosed herein is a compound of Formula I′ or Formula I having the following structure:

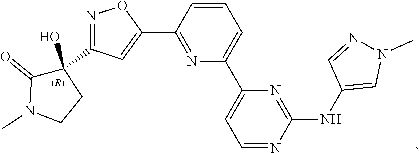

or a pharmaceutically acceptable salt thereof.

In some embodiments, disclosed herein is a compound of Formula I′ or Formula I having the following structure:

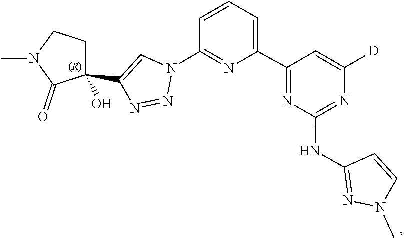

or a pharmaceutically acceptable salt thereof.

In some embodiments, disclosed herein is a compound of Formula I′ or Formula I having the following structure:

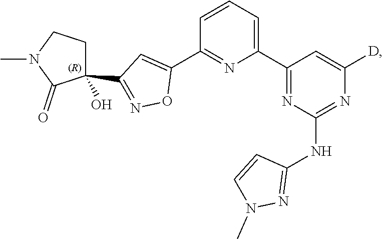

or a pharmaceutically acceptable salt thereof.

In some embodiments, disclosed herein is a compound of Formula I′ or Formula I having the following structure:

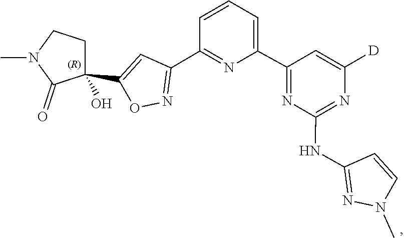

or a pharmaceutically acceptable salt thereof.

In some embodiments, disclosed herein is a compound of Formula I′ or Formula I having the following structure:

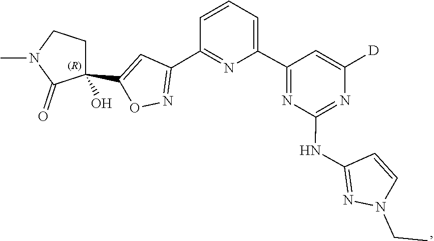

or a pharmaceutically acceptable salt thereof.

In some embodiments, disclosed herein is a compound of Formula I′ or Formula I having the following structure:

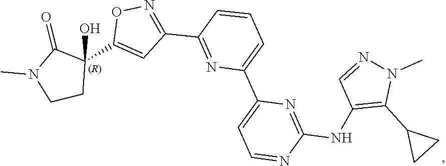

or a pharmaceutically acceptable salt thereof.

In some embodiments, disclosed herein is a compound of Formula I′ or Formula I having the following structure:

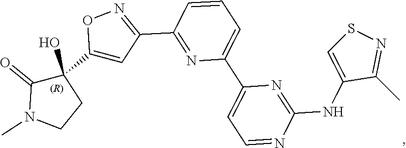

or a pharmaceutically acceptable salt thereof.

In some embodiments, disclosed herein is a compound of Formula I′ or Formula I having the following structure:

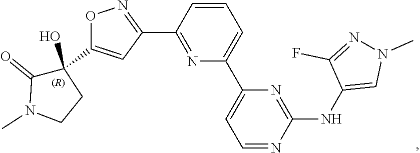

or a pharmaceutically acceptable salt thereof.

In some embodiments, disclosed herein is a compound of Formula I′ or Formula I having the following structure:

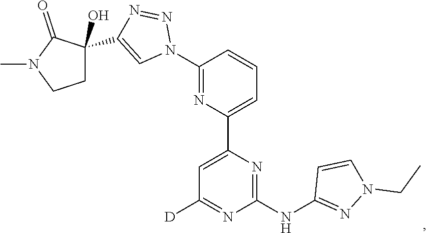 [SMILES: [2H]c1cc(-c2cccc(-n3cc([C@]4(O)CCN(C)C4=O)nn3)n2)nc(Nc2ccn(CC)n2)n1]

or a pharmaceutically acceptable salt thereof.

In some embodiments, disclosed herein is a pharmaceutical composition comprising a compound of Formula I′ or Formula I, or a pharmaceutically acceptable salt thereof, and a pharmaceutically acceptable carrier.

### Therapeutic Use

The present disclosure is also directed toward a method for treating a disease, disorder, or medical condition mediated by NIK activity, comprising administering to a subject in need of such treatment an effective amount of a compound of the disclosure, or a pharmaceutically acceptable salt thereof, or a pharmaceutical composition of the disclosure.

The present disclosure is also directed toward a method for preventing a disease, disorder, or medical condition mediated by NIK activity, comprising administering to a subject in need of such treatment an effective amount of a compound of the disclosure, or a pharmaceutically acceptable salt thereof, or a pharmaceutical composition of the disclosure. In some embodiments, the compound disclosed herein can be used in combination with another therapeutic agent.

The present disclosure is also directed toward a method for improving or ameliorating a symptom of a disease, disorder, or medical condition mediated by NIK activity, comprising administering to a subject in need of such treatment an effective amount of a compound of the disclosure, or a pharmaceutically acceptable salt thereof, or a pharmaceutically acceptable salt thereof.

In some embodiments of the methods of treatment disclosed herein, the disease, disorder, or medical condition is selected from the group consisting of inflammatory disorders and autoimmune disorders.

In some embodiments, the disease, disorder, or medical condition is selected from the group consisting of systemic lupus erythematosus, rheumatoid arthritis, Sjogren's syndrome, and lupus nephritis.

In some embodiments of the methods of treatment disclosed herein, the disease, disorder, or medical condition is selected from the group consisting of inflammatory disorders, autoimmune disorders, cancers, metabolic disorders, and osteoporosis.

In some embodiments of the methods of treatment disclosed herein, the disease, disorder, or medical condition is an autoantibody associated disease. In some embodiments, the autoantibody associated diseases is selected from the group consisting of anti-neutrophil cytoplasmic antibody (“ANCA”) associated vasculitis, scleroderma, Sjogren's disease, myositis, IgG4 associated diseases, bullous pemphigoid, and neuromyelitis optica spectrum disorders (“NMOSD”).

In some embodiments of the methods of treatment disclosed herein, the disease, disorder, or medical condition is an immune mediated dermatitis indication. In some embodiments, the immune mediated dermatitis indication is selected from the group consisting of atopic dermatitis and hidradenitis supperativa.

In some embodiments of the methods of treatment disclosed herein, the disease, disorder, or medical condition is a liver inflammation or a liver injury. In some embodiments, the liver inflammation or a liver injury is selected from the group consisting of steatosis, non-alcoholic steatohepatitis (“NASH”) and primary biliary cirrhosis.

In some embodiments of the methods of treatment disclosed herein, the disease, disorder, or medical condition is cancer. In some embodiments, the cancer is selected from the group consisting of leukemias, lymphomas, pancreatic cancer, breast cancer, and melanoma.

In some embodiments of the methods of treatment disclosed herein, the disease, disorder, or medical condition is a metabolic disorder. In some embodiments, the metabolic disorder is selected from the group consisting of obesity and diabetes. In some embodiments, the diabetes is a type 2 diabetes.

In some embodiments of the methods of treatment disclosed herein, the disease, disorder, or medical condition is a kidney disease. In some embodiments, the kidney disease is selected from the group consisting of acute kidney injury, Berger's disease (IgA nephropathy (IgAN)), autosomal dominant polycystic kidney disease (“ADCKD”), and membranous nephropathy.

In some embodiments of the methods of treatment disclosed herein, the disease, disorder, or medical condition is osteoporosis.

In some embodiments of the methods of treatment disclosed herein, the disease, disorder, or medical condition is selected from the group consisting of RA, IBD, SLE, IgAN, metabolic syndrome, multiple sclerosis, Immune thrombocytopenic purpura, primary biliary cirrhosis, transplantation, myasthenia gravis, osteoporosis, and bone resorption (periodontitis).

In some embodiments of the methods of treatment disclosed herein, the disease, disorder, or medical condition is selected from the group consisting of systemic lupus erythematosus (“SLE”), rheumatoid arthritis (“RA”), Sjogren's syndrome, lupus nephritis, inflammatory bowel disease (“IBD”), ANCA associated vasculitis, myositis, IgG4 associated diseases, bullous pemphigoid, neuromyelitis optica spectrum disorders (“NMOSD”), atopic dermatitis “AD”), hidradenitis supperativa (“HS”), steatosis, non-alcoholic steatohepatitis (“NASH”), primary biliary cirrhosis, leukemias, lymphomas, pancreatic cancer, breast cancer, melanoma, obesity, diabetes, acute kidney injury, IgAN, autosomal dominant polycystic kidney disease (“ADCKD”), membranous nephropathy, osteoporosis, bone resorption (periodontitis), multiple sclerosis (“MS”), immune thrombocytopenic purpura, transplantation, myasthenia gravis, scleroderma, myositis, IgG4 associated diseases, and bullous pemphigoid.

In some embodiments, disclosed herein is a method for preventing or controlling an excessive inflammatory response, comprising administering to a subject in need of such treatment an effective amount of a compound of the disclosure, or a pharmaceutically acceptable salt thereof, or a pharmaceutical composition of the disclosure.

In some embodiments of the methods disclosed herein, the subject is a human subject.

The present disclosure also provides a method for modulating NIK activity, comprising exposing NIK to an effective amount of a compound of the disclosure, or a pharmaceutically acceptable salt thereof.

In some embodiments, the disclosure provides a method for inhibiting NIK activity, comprising exposing NIK to an effective amount of a compound of the disclosure, or a pharmaceutically acceptable salt thereof.

### Dosing and Administration

In the methods disclosed herein, an effective amount of at least one compound according to the disclosure is administered to a subject suffering from or diagnosed as having such a disease, disorder, or medical condition. An “effective amount” means an amount or dose sufficient to generally bring about the desired therapeutic or prophylactic benefit in patients in need of such treatment for the designated disease, disorder, or medical condition. For a 70-kg human, an illustrative range for a dosage amount is from about 1 to 1000 mg/day in single or multiple dosage units.

In some embodiments, the dosage amount is about 1 mg to 500 mg of a compound of the disclosure, or a pharmaceutically acceptable salt thereof. In some embodiments, the dosage amount is about 1, 5, 10, 15, 20, 25, 30, 35, 40, 45, 50, 55, 60, 65, 70, 75, 80, 85, 90, 95, or 100 mg of a compound of the disclosure, or pharmaceutically acceptable salt thereof. In some embodiments, the dosage amount is about 100, 105, 110, 115, 120, 125, 130, 135, 140, 145, 150, 155, 160, 165, 170, 175, 180, 185, 190, 195, or 200 mg of a compound of the disclosure, or pharmaceutically acceptable salt thereof. In some embodiments, the dosage amount is about 200, 205, 210, 215, 220, 225, 230, 235, 240, 245, 250, 255, 260, 265, 270, 275, 280, 285, 290, 295, or 300 mg of a compound of the disclosure, or pharmaceutically acceptable salt thereof. In some embodiments, the dosage amount is about 300, 315, 320, 325, 330, 335, 340, 345, 350, 355, 360, 365, 370, 375, 380, 385, 390, 395, or 400 mg of a compound of the disclosure, or pharmaceutically acceptable salt thereof. In some embodiments, the dosage amount is about 400, 405, 410, 415, 420, 425, 430, 435, 440, 445, 450, 455, 460, 465, 470, 475, 480, 485, 490, 495, or 500 mg of a compound of the disclosure, or pharmaceutically acceptable salt thereof

The dosage administered will be affected by factors such as the route of administration, the health, weight and age of the recipient, the frequency of the treatment and the presence of concurrent and unrelated treatments.

It is also apparent to one skilled in the art that the therapeutically effective dose for compounds of the present disclosure or a pharmaceutical composition thereof will vary according to the desired effect. Therefore, optimal dosages to be administered may be readily determined by one skilled in the art and will vary with the particular compound used, the mode of administration, the strength of the preparation, and the advancement of the disease condition. In addition, factors associated with the particular subject being treated, including subject age, weight, diet and time of administration, will result in the need to adjust the dose to an appropriate therapeutic level. The above dosages are thus exemplary of the average case. There can, of course, be individual instances where higher or lower dosage ranges are merited, and such are within the scope of the present disclosure.

Once improvement of the patient's disease, disorder, or condition has occurred, the dose may be adjusted for preventive or maintenance treatment. For example, the dosage or the frequency of administration, or both, may be reduced as a function of the symptoms, to a level at which the desired therapeutic or prophylactic effect is maintained. Of course, if symptoms have been alleviated to an appropriate level, treatment may cease. Patients may, however, require intermittent treatment on a long-term basis upon any recurrence of symptoms.

The compounds of the present disclosure, or pharmaceutically acceptable salts thereof, may be formulated into pharmaceutical compositions comprising any known pharmaceutically acceptable carriers. Pharmaceutically acceptable carriers commonly used in pharmaceutical compositions are substances that are non-toxic, biologically tolerable, and otherwise biologically suitable for administration to a subject, such as an inert substance, added to a pharmacological composition or otherwise used as a vehicle or diluent to facilitate administration of an agent and that is compatible therewith. Exemplary carriers include, but are not limited to, any suitable solvents, dispersion media, coatings, antibacterial and antifungal agents and isotonic agents. Exemplary excipients that may also be components of the formulation include fillers, binders, disintegrating agents and lubricants.

Delivery forms of the pharmaceutical compositions containing one or more compounds of the disclosure may be prepared using pharmaceutically acceptable excipients and compounding techniques known or that become available to those of ordinary skill in the art. The compositions may be administered in the disclosed methods by a suitable route of delivery, e.g., oral, parenteral, rectal, topical, or ocular routes, or by inhalation.

The preparation may be in the form of tablets, capsules, sachets, dragees, powders, granules, lozenges, powders for reconstitution, liquid preparations, or suppositories. The compositions may be formulated for any one of a plurality of administration routes, such as intravenous infusion, subcutaneous injection, topical administration, or oral administration.

For oral administration, the compounds of the present disclosure can be provided in the form of tablets, capsules, or beads, or as a solution, emulsion, or suspension. To prepare the oral compositions, the active agents may be formulated to yield a dosage of, e.g., for a 70-kg human, from about 1 to 1000 mg/day in single or multiple dosage units as an illustrative range.

Oral tablets may include a compound of the disclosure mixed with compatible pharmaceutically acceptable excipients such as diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Illustrative examples of liquid oral excipients include ethanol, glycerol, water, and the like. Starch, polyvinyl-pyrrolidone (PVP), sodium starch glycolate, microcrystalline cellulose, and alginic acid are examples of disintegrating agents. Binding agents may include starch and gelatin. The lubricating agent, if present, may be magnesium stearate, stearic acid or talc. If desired, the tablets may be coated with a material such as glyceryl monostearate or glyceryl distearate to delay absorption in the gastrointestinal tract or may be coated with an enteric coating. Additional coatings that may be used include coatings that are designed to release the compound or active agent as a function of time, pH or bacterial content.

Capsules for oral administration include hard and soft gelatin or (hydroxypropyl)methyl cellulose capsules. To prepare hard gelatin capsules, active ingredient(s) may be mixed with a solid, semi-solid, or liquid diluent. Soft gelatin capsules may be prepared by mixing the active ingredient with an oil such as peanut oil or olive oil, liquid paraffin, a mixture of mono and di-glycerides of short chain fatty acids, polyethylene glycol 400, or propylene glycol. Liquids for oral administration may be in the form of suspensions, solutions, emulsions or syrups or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

The compounds of the present disclosure may also be administered by non-oral routes. For example, compositions may be formulated for rectal administration as a suppository, enema or foam. For parenteral use, including intravenous, intramuscular, intraperitoneal, or subcutaneous routes, the compounds of the disclosure may be provided in sterile aqueous solutions or suspensions, buffered to an appropriate pH and isotonicity or in parenterally acceptable oil. Suitable aqueous vehicles include Ringer's solution and isotonic sodium chloride. Such forms may be presented in unit-dose form such as ampules or disposable injection devices, in multi-dose forms such as vials from which the appropriate dose may be withdrawn, or in a solid form or pre-concentrate that can be used to prepare an injectable formulation. Illustrative infusion doses range from about 1 to 1000 μg/kg/minute of agent admixed with a pharmaceutical carrier over a period ranging from several minutes to several days.

For topical administration, the compounds of the disclosure may be mixed with a pharmaceutical carrier. Another mode of administering the compounds of the disclosure may utilize a patch formulation to effect transdermal delivery.

Compounds of the disclosure may alternatively be administered in methods of the present disclosure by inhalation, via the nasal or oral routes, e.g., in a spray formulation also containing a suitable carrier.

Although the present embodiments have been described in connection with certain specific embodiments for instructional purposes, the present embodiments are not limited thereto. Accordingly, various modifications, adaptations, and combinations of various features of the described embodiments can be practiced without departing from the scope of the invention as set forth in the claims. Furthermore, the following examples are illustrative, but not limiting, of the compounds, compositions and methods described herein. Other suitable modifications and adaptations known to those skilled in the art are within the scope of the following embodiments. Any and all journal articles, patent applications, issued patents, or other cited references are incorporated by reference in their entirety

### EXAMPLES

The following specific examples are provided to further illustrate embodiments within the scope of the present disclosure.

### Abbreviations

Herein and throughout the application, the following abbreviations may be used.

| | |
|---|---|
| Ac | acyl or acetyl |
| ACN or MeCN | acetonitrile |
| AcOH or HOAc | acetic acid |
| br | broad |
| BrettPhos | 2-(Dicyclohexylphosphino)3,6-dimethoxy-2′,4′,6′- |
| | triisopropyl-1,1′-biphenyl |
| BrettPhos Pd G3 | [(2-Di-cyclohexylphosphino-3,6-dimethoxy-2′,4′,6′- |
| | triisopropyl-1,1′-biphenyl)-2-(2′-amino-1,1′- |
| | biphenyl)]palladium(II) methanesulfonate |
| | methanesulfonate |
| n-BuLi | n-butyllithium |
| Bu | butyl |
| d | doublet |
| DAC | dynamic axial compression |
| DAST | diethylaminosulfur trifluoride |
| DBAD | di-tert-butyl azodicarboxylate |
| DBU | 1,8-diazabicyclo(5.4.0)undec-7-ene |
| DCE | dichloroethane |
| DCM | dichloromethane |
| DEA | diethylamine |
| DIAD | diisopropyl azodicarboxylate |
| DIEA | N,N-diisopropylethylamine |
| DIPEA | N,N-diisopropylethylamine |
| DMA | dimethylacetamide |
| DMAP | 4-dimethylaminopyridine |
| DMF | N,N-dimethylformamide |
| DMSO | dimethyl sulfoxide |
| ESI | electrospray ionization |
| Et | Ethyl |
| EtMgBr | ethyl magnesium bromide |
| Et₃N or TEA | triethylamine |
| Et₂NH | diethylamine |
| Et₂O | diethyl ether |
| EtOAc or EA | ethyl acetate |
| EtOH | ethanol |
| FCC | flash column chromatography |
| h or hr | hour(s) |
| HATU | 1-[bis(dimethylamino)methylene]-1H-1,2,3-triazolo[4,5- |
| | b]pyridinium 3-oxid hexafluorophosphate |
| HBTU | O-(benzotriazol-1-yl)-N,N,N′, N′-tetramethyluronium |
| | hexafluorophosphate |
| HPLC | high pressure liquid chromatography |
| Hz | Hertz |
| i | iso |
| IPA or i-PrOH | isopropanol |
| Josiphos Pd G3 | {(R)-1-[(Sp)-2-(dicyclohexylphosphino)ferrocenyl]ethyldi- |
| | tert-butylphosphine}[2-(2′-amino-1,1′- |
| | biphenyl)]palladium(II) methanesulfonate |
| Lawesson's reagent | 2,4-Bis(4-methoxyphenyl)-1,3,2,4-dithiadiphosphetane- |
| | 2,4-disulfide |
| LCMS | liquid chromatography mass spectrometry |
| KOAc | potassium acetate |
| LDA | lithium diisopropylamide |
| LiHMDS | lithium bis(trimethylsilyl)amide |
| m | multiplet |
| mCPBA or m-CPBA | meta-chloroperoxybenzoic acid |
| mmol | millimoles |
| m/z | mass-to-charge ratio |
| M | molar |
| M+ | parent molecular ion |
| Me | methyl |
| MeNH₂ | methylamine |
| MeOH | methanol |
| MeTHF | 2-methyltetrahydrofuran |
| min | minute(s) |
| MP-TMT | ^{resin bound 2,4,6-trimercaptotriazine} |
| MS | mass spectrometry |
| MTBE or TBME | methyl tert-butyl ether |
| NaHMDS | sodium bis(trimethylsilyl)amide |
| NaSO₂Me | sodium methyl sulfate |
| NCS | N-chlorosuccinimide |
| NBS | N-bromosuccinimide |
| NMP | N-methyl-2-pyrrolidone |
| NMR | nuclear magnetic resonance |
| nt | not tested |
| p | pentet |
| Pd₂(dba)₃ | tris(dibenzylideneacetone)dipalladium(0) |
| Pd(dppf)Cl₂ or | [1,1′- |
| PdCl₂(dppf) | bis(diphenylphosphino)ferrocene]dichloropalladium(II) |
| Pd(dtbpf)Cl₂ | [1,1-bis(di-tert- |
| | butylphosphino)ferrocene]dichloropalladium(II) |
| Pd(PPh₃)₄ | tetrakis(triphenylphosphine)palladium(0) |
| Pd(PPh₃)₂Cl₂ | bis(triphenylphosphine)palladium chloride |
| Pd(OAc)₂ | palladium(II)acetate |
| Pd(t-Bu₃P)₂ | bis(tri-tert-butylphosphine)palladium(0) |
| PE or pet ether | petroleum ether |
| psi | pounds per square inch |
| q | quartet |
| RP | reversed-phase |
| rac-binap-Pd-G3 | [2′-(amino-κN)[1,1′-biphenyl]-2-yl-κC][[2′- |
| | (diphenylphosphino)[1,1′-binaphthalen]-2- |
| | yl]diphenylphosphine-κP](methanesulfonato-κO) |
| | palladium |
| rt | room temperature |
| S | singlet |
| sat. | saturated |
| SFC | supercritical fluid chromatography |
| Select-F | selectfluor |
| SEM-Cl | 2-(trimethylsilyl)ethoxymethyl chloride |
| (SnBu₃)₂ | bis(tributyltin) |
| t | tert or triplet |
| TBAF | tetrabutylammonium fluoride |
| TBSCl | tert-butyldimethylsilyl chloride |
| t-BuBrettphos | 2-(di-tert-butylphosphino)-2′,4′,6′-triisopropyl-3,6- |
| | dimethoxy-1,1′-biphenyl |
| t-BuBrettphos Pd G3 | [(2-di-tert-butylphosphino-3,6-dimethoxy-2′,4′,6′- |
| | triisopropyl-1,1′-biphenyl)-2-(2′-amino-1,1′- |
| | biphenyl)]palladium(II) methanesulfonate |
| t-BuOK | potassium tert-butoxide |
| TEA | triethylamine |
| TFA | trifluoroacetic acid |
| THF | tetrahydrofuran |
| TLC | thin layer chromatography |
| TMPMgCl•LiCl | 2,2,6,6-tetramethylpiperidinylmagnesium chloride lithium |
| | chloride complex |
| TMS | trimethylsilane |
| TosCl or TsCl | p-toluenesulfonyl chloride |
| TsOH | p-toluenesulfonic acid |
| v/v | volume-to-volume ratio |
| Xantphos | 4,5-bis(diphenylphosphino)-9,9-dimethylxanthene |
| Xantphos Pd G3 | [(4,5-bis(diphenylphosphino)-9,9-dimethylxanthene)-2- |
| | (2′-amino-1,1′-biphenyl)]palladium(II) methanesulfonate |
| — | Not tested |

In some embodiments, provided herein are processes and intermediates disclosed herein that are useful for preparing a compound of the disclosure or pharmaceutically acceptable salts thereof.

By way of illustration, but not as limitation, compounds of the present disclosure, compounds of Formula I′ or Formula I, are prepared according to the following general preparation procedures given by Schemes 1-6. One of ordinary skill in the art will recognize that, to obtain the various compounds herein, starting materials may be suitably selected so that the ultimately desired substituents will be carried through the reaction scheme with or without protection as appropriate to yield the desired product. Alternately, in the place of the ultimately desired substituent, a suitable group may be carried through the reaction scheme and replaced, as appropriate, with the desired substituent. Unless otherwise specified, the variables in Schemes 1-6 are as defined above in reference to Formula I′ or Formula I.

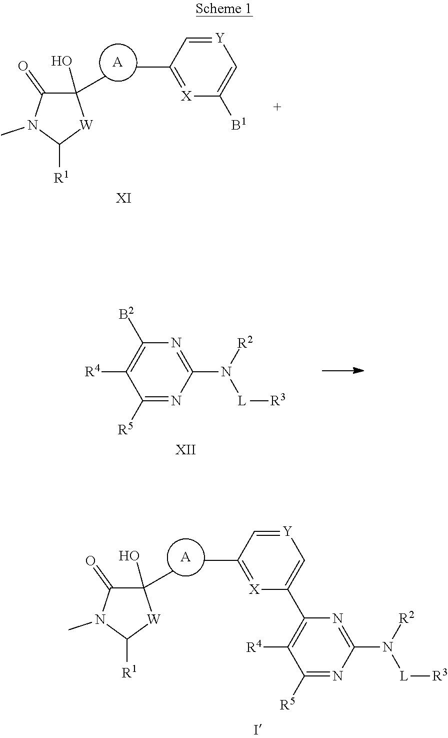

The compounds of Formula I′ of the present disclosure can be prepared, for example, as shown in Scheme 1. When substituent B¹ in compound XI is a halide (e.g., Cl, Br, or I) and when substituent B² in compound XII is a stannyl group (e.g., Sn(Me)₃ or Sn(n-Bu)₃), this coupling is achieved by reaction under Stille conditions. One having skill in the art would recognize that coupling under Stille conditions also provides a compound of Formula I′ when B¹ in compound XI is a stannyl group and B² in compound XII is a halide. Typical Stille coupling conditions involve the use of a catalyst (usually palladium, but sometimes nickel), a suitable solvent, and other optional reagents such as CuI and TEA. Examples of suitable catalysts include, but are not limited to Pd(dppf)Cl₂, Pd(PPh₃)₄, and Pd(P(Cy)₃)₂Cl₂. Suitable solvents include, but are not limited to, 1,4-dioxane, toluene, and DMF. The reaction may be heated to a temperature from about 100° C. to about 120° C. for a time period of about 1 to 16 hours, employing microwave or conventional heating to provide the compound of Formula I′.

Alternately, Suzuki coupling conditions can be used to couple compound XI to compound XII when B¹ is a boron-based coupling agent such as a boronic acid or a boronic ester and when B² is a halide such as Cl, Br, or I. One having skill in the art would recognize that coupling under Suzuki conditions also provides a compound of Formula I′ when B¹ is a halide and when B² is a boron-based coupling agent. Typical Suzuki coupling conditions involve the use of a palladium catalyst (e.g., Pd(dtbpf)Cl₂), a base (e.g., K₃PO₄), and a suitable solvent (e.g., 1,4 dioxane, water, or a mixture thereof). The reaction may be heated to a temperature of about 80° C. for a time period of about 1 hour, employing microwave or conventional heating to provide the compound of Formula I′.

Alternately, Buchwald coupling conditions can be used to couple compound XI to compound XII when B¹ is a boron-based coupling agent such as a boronic acid or a boronic ester and when B² is a halide such as Cl, Br, or I

Where a protecting group is present on a compound of Formula XI or XII, a final deprotection step is added, employing conditions known to one skilled in the art, to provide the compound of Formula I′. For example, if a (trimethylsilyl)ethoxymethyl (SEM) group is used to protect a nitrogen atom, it can be removed using a reagent such as TFA in a solvent such as DCM.

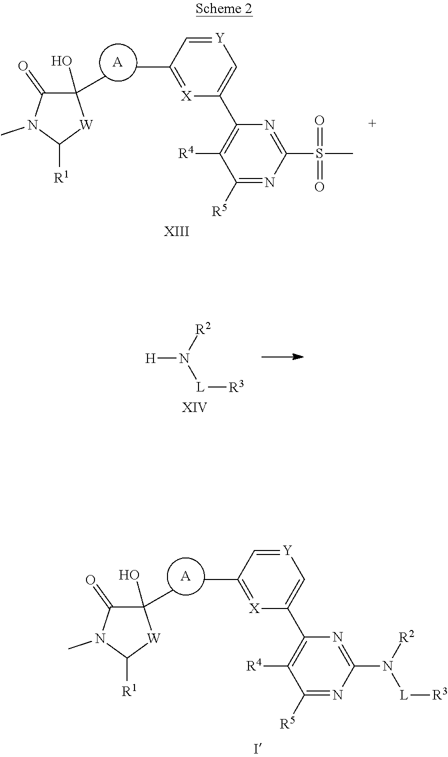

The compounds of Formula I′ of the present disclosure can be prepared, for example, as shown in Scheme 2. Compounds XIII and XIV may be combined with a suitable acid, such as TFA or TsOH, in a solvent such as DMSO or 1,4-dioxane. The reaction may be heated to a temperature from about 115° C. to about 150° C. for a time period of about 1.5 to 16 hours to provide the compound of Formula I′.

Alternately, compounds XIII and XIV may be combined with a suitable base, such as TEA or DIPEA, in a solvent such as DMSO or DMA. The reaction mixture may be heated to a temperature from about 120° C. to about 150° C. for a time period of about 1.5 to 3.5 hours to provide the compound of Formula I′.

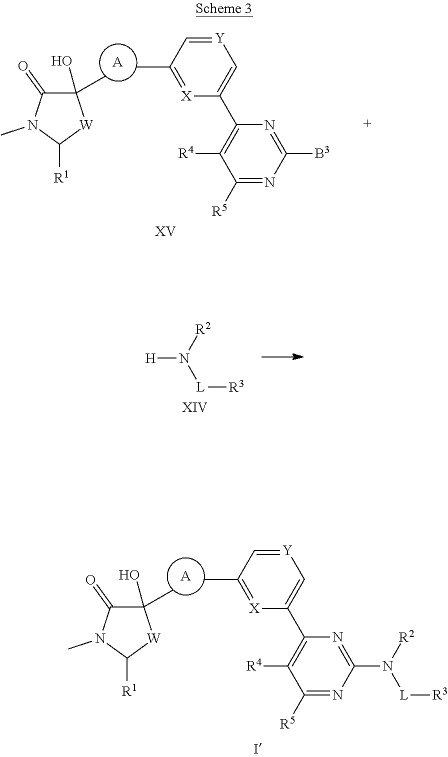

The compounds of Formula I′ of the present disclosure can be prepared, for example, as shown in Scheme 3. When substituent B³ in compound XV is a halide (e.g., Cl, Br, or I), preparation of the compound of Formula I′ can be achieved under Buchwald-Hartwig amination conditions. Typical Buchwald-Hartwig reactions involve the use of a catalyst (usually palladium e.g., Pd₂(dba)₃, but sometimes other metals); a base (e.g., Cs₂CO₃), and a suitable solvent (e.g., THF). A phosphate ligand, such as e.g., 4,5-bis(diphenylphosphino)-9,9-dimethylxanthene, may also be included. The reaction may be heated to a temperature of about 120° C. for a time period of about 16 hours to provide the compound of Formula I′.

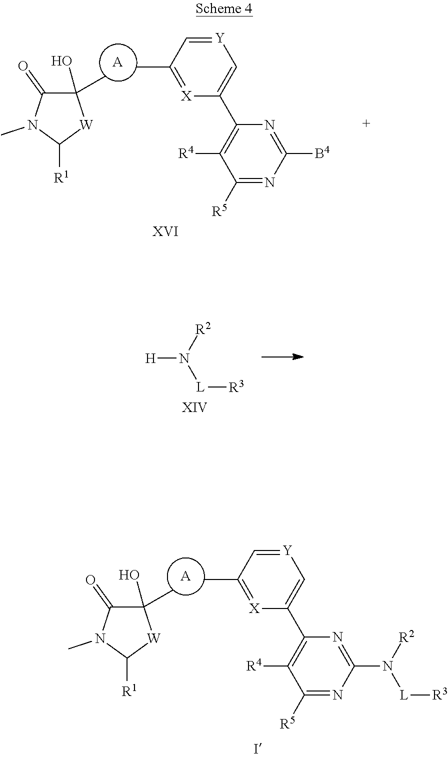

The compounds of Formula I′ of the present disclosure can be prepared, for example, as shown in Scheme 4. Substituent B⁴ in compound XVI is, for example, a halide such as F or Cl. Compounds XVI and XIV may be combined with a suitable base, such as DIPEA, in a solvent such as DMA or NMP. The reaction may be heated to a temperature from about 80° C. to 150° C. for a time period of 1 hour, employing microwave or conventional heating, to provide the compound of Formula I′.

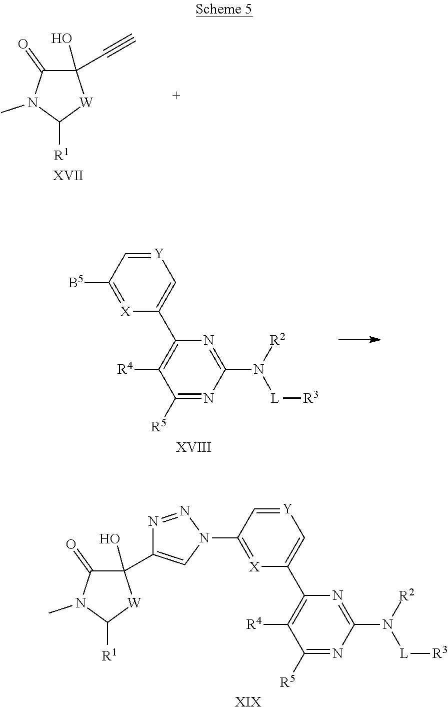

The compounds of Formula I′ of the present disclosure, where A is a triazolyl ring, can be prepared, for example, as shown in Scheme 5. Substituent B⁵ in compound XVIII is, for example, a halide such as Cl, Br, or I. Compounds XVII and XVIII may be combined with a copper catalyst (e.g., CuI), a ligand (e.g., (1 S,2S)—N¹,N²-dimethylcyclohexane-1,2-diamine), and NaN₃ in a solvent such as DMSO. An additive, such as sodium ascorbate may also be included. The reaction may be allowed to progress at room temperature for about 16 hours to provide the compound of Formula XIX.

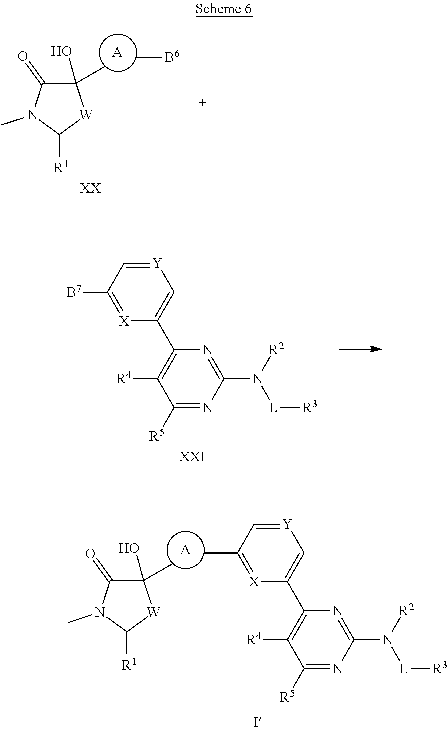

The compounds of Formula I′ of the present disclosure can be prepared, for example, as shown in Scheme 6. When substituent B⁶ in compound XX is a halide (e.g., Cl, Br, or I) and when substituent B⁷ in compound XXI is a stannyl group (e.g., Sn(Me)₃ or Sn(n-Bu)₃), this coupling is achieved by reaction under Stille conditions. One having skill in the art would recognize that coupling under Stille conditions also provides a compound of Formula I′ when B⁶ in compound XX is a stannyl group and B⁷ in compound XXI is a halide. Typical Stille coupling conditions involve the use of a catalyst (usually palladium, but sometimes nickel), a suitable solvent, and other optional reagents such as CuI and TEA. Examples of suitable catalysts include, but are not limited to Pd(dppf)Cl₂, Pd(PPh₃)₄, and Pd(P(Cy)₃)₂Cl₂. Suitable solvents include, but are not limited to, 1,4-dioxane, toluene, and DMF. The reaction may be heated to a temperature from about 100° C. to about 120° C. for a time period of about 1 to 16 hours, employing microwave or conventional heating to provide the compound of Formula I′.

Alternately, Suzuki coupling conditions can be used to couple compound XX to compound XXI when B⁶ is a boron-based coupling agent such as a boronic acid or a boronic ester and when B⁷ is a halide such as Cl, Br, or I. One having skill in the art would recognize that coupling under Suzuki conditions also provides a compound of Formula I′ when B⁶ is a halide and when B⁷ is a boron-based coupling agent. Typical Suzuki coupling conditions involve the use of a palladium catalyst (e.g., Pd(dtbpf)Cl₂), a base (e.g., K₃PO₄), and a suitable solvent (e.g., 1,4 dioxane, water, or a mixture thereof). The reaction may be heated to a temperature of about 80° C. for a time period of about 1 hour, employing microwave or conventional heating to provide the compound of Formula I′.

Where a protecting group is present on a compound of Formula XX or XXI, a final deprotection step is added, employing conditions known to one skilled in the art, to provide the compound of Formula I′. For example, if a (trimethylsilyl)ethoxymethyl (SEM) group is used to protect a nitrogen atom, it can be removed using a reagent such as TFA in a solvent such as DCM.

Where compounds are obtained as a salt, they can be converted to the corresponding free base by techniques known in the art. For example, those compounds obtained as a TFA salt could be converted to the corresponding free base by methods such as passage through a carbonate cartridge.

In obtaining the compounds described in the examples below and the corresponding analytical data, the following experimental and analytical protocols were followed unless otherwise indicated.

Unless otherwise specified, reaction solutions were stirred at room temperature under a N_{2(g)} or Ar_{(g)} atmosphere. When solutions were “concentrated to dryness”, they were concentrated using a rotary evaporator under reduced pressure. When solutions were dried, they are typically dried over a drying agent such as MgSO₄ or Na₂SO₄. Normal phase flash column chromatography (FCC) was performed on silica gel with prepackaged silica gel columns, such as RediSep®, using ethyl acetate (EtOAc)/hexanes, CH₂Ck₂/MeOH, or CH₂Cl₂/10% 2N NH₃ in MeOH, as eluent, unless otherwise indicated.

The compounds described in the examples may also be purified via preparative reverse-phase HPLC. A typical HPLC chromatographic separation ranges from about 10 to about 20 minutes. Suitable solvent gradients and conditions for purification may be determined by one having skill in the art. Unless otherwise specified, where reverse-phase HPLC is used to purify a compound described in one of the examples below, the solvent used is a gradient of 10% to 80% acetonitrile in water. When the purification is performed under “acidic conditions” or in “acidic media,” the acetonitrile and water both contain 0.16% TFA. When performed under “basic conditions” or in “basic media,” the pH of the acetonitrile and water have been adjusted to pH 10 with ammonium hydroxide. The following column abbreviations are also used throughout the examples:

| | Column Type |
|---|---|
| C1 | Waters XSelect CSH C18, 5 μm, 19 × 100 mm |
| C2 | Waters XBridge BEH C18, 5 μm, 19 × 100 mm |
| C3 | Waters XSelect CSH C18, 5 μm, 19 × 150 mm |
| C4 | Waters XBridge BEH C18, 5 μm, 19 × 150 mm |
| C5 | Waters XSelect CSH Fluoro Phenyl, 5 μm, 19 × 100 mm |
| C6 | Waters XSelect CSH Fluoro Phenyl, 5 μm, 19 × 150 mm |
| C7 | Waters XSelect CSH C18, 5 μm, 30 × 150 mm |
| C8 | Waters XBridge BEH C18, 5 μm, 30 × 150 mm |

| Descriptor | Mobile Phases |
|---|---|
| acidic conditions/ | water containing 0.16% TFA and |
| acidic media | acetonitrile containing 0.16% TFA |
| basic conditions/ | water at pH 10 with ammonium |
| basic media | hydroxide and acetronitrile at pH 10 |
| | ammonium hydroxide |

Thin-layer chromatography was performed using silica gel plates, such as Merck silica gel 60 F₂₅₄ 2.5 cm×7.5 cm 250 mm or 5.0 cm×10.0 cm 250 μm pre-coated silica gel plates. Preparative thin-layer chromatography was performed using silica gel plates such as EM Science silica gel 60 F₂₅₄ 20 cm×20 cm 0.5 mm pre-coated plates with a 20 cm×4 cm concentrating zone. Microwave reactions were carried out in a microwave reactor, such as a CEM Discover^{á}, a Biotage Initiator™ or Optimizer™ microwave, at specified temperatures. Mass spectra were obtained on a mass spectrometer, such as Agilent series 1100 MSD using electrospray ionization (ESI) in positive mode unless otherwise indicated. Calculated mass corresponds to the exact mass. NMR spectra were obtained on an NMR spectrometer, such as a Bruker model DPX400 (400 MHz), DPX500 (500 MHz), DRX600 (600 MHz) spectrometer. The format of the ¹H NMR data below is as follows: Chemical shift in ppm down field of the tetramethylsilane reference (multiplicity, coupling constant J in Hz, integration).

Whenever a yield is given as a percentage, such yield refers to a mass of the entity for which the yield is given with respect to the maximum amount of the same entity that could be obtained under the particular stoichiometric conditions. Reagent concentrations that are given as percentages refer to mass ratios, unless indicated differently. Whether expressly indicated or not, yields given in the following examples are computed with respect to the dried form of the compound for which any such yield is given.

Chemical names were generated using ChemDraw Ultra 17.1 (CambridgeSoft Corp., Cambridge, MA) or OEMetaChem V1.4.0.4 (Open Eye).

### Intermediate 1: (R)-3-Ethynyl-3-hydroxy-1-methylpyrrolidin-2-one-4,4,5,5-d₄

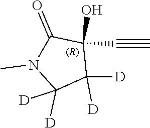

Step A: 1-Methylpyrrolidin-2-one-3,3,4,4,5,5-d₆. Into a 1000-mL, 3-necked round-bottom flask, purged and maintained with an atmosphere of nitrogen, was placed NaH (60% in mineral oil, 52 g, 1302 mmol). THF (400 mL) was added and the mixture was then cooled to 0° C. To this was added pyrrolidin-2-one-3,3,4,4,5,5-d₆ (39.5 g, 434 mmol) at 0° C. The resulting solution was stirred at 0° C. for 20 min, then iodomethane (184 g, 1302 mmol) was added dropwise at 0° C. The resulting solution was slowly warmed to room temperature and stirred for 3.5 hours. The resulting solution was poured into ice-water (800 mL), extracted with DCM (15×400 mL), the organic extracts combined, dried over anhydrous magnesium sulfate and concentrated under reduced pressure to afford 1-methylpyrrolidin-2-one-3,3,4,4,5,5-d6 as a yellow oil (67.6 g). LC-MS (ESI): Mass calcd. for C₅H₃D₆NO 105.1 m/z found 106 [M+H]⁺.

Step B: 3-Benzoyl-1-methylpyrrolidin-2-one-4,4,5,5-d₄. Into a 2000-mL, 3-necked round-bottom flask, purged and maintained with an atmosphere of nitrogen, was added NaH (60% in mineral oil, 26 g, 651 mmol), toluene (900 mL) and methanol (1.82 mL). To this was added a mixture solution of 1-methylpyrrolidin-2-one-3,3,4,4,5,5-d₆ (67.6 g, 434 mmol) and methyl benzoate (65 g, 477 mmol) dropwise at room temperature. The resulting solution was heated at 110° C. overnight. After this time, the reaction mixture was cooled to 0° C. and HOAc (39 g, 651 mmol) and water (24 g, 1302 mmol) were added. After 10 min water was added and the reaction mixture was extracted with 3×600 mL of DCM. The organic extracts were combined, dried over anhydrous sodium sulfate and concentrated under reduced pressure. The product was purified by silica gel chromatography (eluent: PE/EtOAc from 10/1 to 3/1). This resulted in 70.5 g (steps A and B, 78%) of 3-benzoyl-1-methylpyrrolidin-2-one-4,4,5,5-d₄ as a yellow oil. LC-MS (ESI): Mass calcd. for C₁₂H₉D₄NO₂ 207.1 m/z found 208 [M+H]⁺.

Step C: 3-Benzoyl-3-ethynyl-1-methylpyrrolidin-2-one-4,4,5,5-d₄. Into a 2000 mL, 4-necked round-bottom flask with a mechanical agitator, purged and maintained with an atmosphere of nitrogen, was placed 3-benzoyl-1-methylpyrrolidin-2-one-4,4,5,5-d₄ (70.5 g, 340 mmol) and THF (dry, 1200 mL). The resulting mixture was cooled to −78° C., and 1-((trimethylsilyl)ethynyl)-1λ3-benzo[d][1,2]iodaoxol-3(1H)-one (175 g, 510 mmol) was added in one portion and the reaction mixture was stirred at −78° C. for 10 min. TBAF (1 M in THF, 510 mL, 510 mmol) was added dropwise at −78° C. The resulting solution was stirred at −70° C. for 2 hours, and then warmed to room temperature for another 2 hours. The resulting mixture was quenched with saturated NH₄Cl solution, extracted with 3×600 mL of EtOAc and the organic extracts combined, dried over anhydrous sodium sulfate and concentrated under reduced pressure. The product was purified by silica gel chromatography (PE/EtOAc from 10/1 to 5/1) to afford 53.8 g (62%) of 3-benzoyl-3-ethynyl-1-methylpyrrolidin-2-one-4,4,5,5-d₄ as a yellow oil. LC-MS (ESI): Mass calcd. for C₁₄H₉D₄NO₂ 231.1 m/z found 232 [M+H]⁺.

Step D: 3-Ethynyl-1-methyl-2-oxopyrrolidin-3-yl-4,4,5,5-d₄ benzoate. Into a 5000-mL 4-necked round-bottom flask with mechanical agitator, was placed 3-benzoyl-3-ethynyl-1-methylpyrrolidin-2-one-4,4,5,5-d₄ (53.8 g, 233 mmol), DCM (3000 mL), KHCO₃ (116 g, 1165 mmol), and m-CPBA (236 g, 1165 mmol). The resulting mixture was stirred at room temperature overnight. The reaction mixture was then quenched with saturated NaHCO₃ solution, stirred for 1 hour, extracted with 2×1000 mL of DCM and the organic extracts combined, dried over anhydrous sodium sulfate and concentrated under reduced pressure. The product was purified by silica gel chromatography (PE/EtOAc from 10/1 to 3/1) to provide 27 g (47%) of 3-ethynyl-1-methyl-2-oxopyrrolidin-3-yl-4,4,5,5-d₄ benzoate as a yellow solid. LC-MS (ESI): Mass calcd. for C₁₄H₉D₄NO₃ 247.1 m/z found 248 [M+H]⁺.

Step E: 3-Ethynyl-3-hydroxy-1-methylpyrrolidin-2-one-4,4,5,5-d₄. Into a 1000-mL, 4-necked round-bottom flask, was placed 3-ethynyl-1-methyl-2-oxopyrrolidin-3-yl-4,4,5,5-d₄ benzoate (27 g, 109 mmol) and THF (270 mL) and the mixture was cooled to 0° C. A solution of LiOH—H₂O (13.8 g, 327 mmol) in H₂O (270 mL) at 0° C.-10° C. was added and the reaction mixture was stirred at room temperature for 5 hours. The mixture was then cooled to 0° C., the pH was adjusted to pH=7 with 1M HCl, and the reaction mixture was concentrated under reduced pressure. The product was purified by silica gel chromatography (DCM/MeOH from 300/1 to 100/1) to afford 15.1 g (87%) of 3-ethynyl-3-hydroxy-1-methylpyrrolidin-2-one-4,4,5,5-d₄ as a yellow solid. LC-MS (ESI): Mass calcd. for C₇H₅D₄NO₂ 143.1 m/z found 144 [M+H]⁺.

Step F: (R)-3-Ethynyl-3-hydroxy-1-methylpyrrolidin-2-one-4,4,5,5-d₄. The (R) and (S) enantiomers of 3-ethynyl-3-hydroxy-1-methylpyrrolidin-2-one-4,4,5,5-d₄ were separated with SFC (Column: CHIRALPAK® IH, 3*25 cm, 5 μm; Mobile Phase A: CO₂, Mobile Phase B: IPA (0.5% 2M NH₃-MeOH); Flow rate: 100 mL/min; Gradient: 10% B; Column Temperature: 35° C.; Back Pressure: 100 bar; 220 nm; RT1:3.62; RT2:4.82). The first eluting peak was (S)-3-ethynyl-3-hydroxy-1-methylpyrrolidin-2-one-4,4,5,5-d₄ and the second eluting peak was (R)-3-ethynyl-3-hydroxy-1-methylpyrrolidin-2-one-4,4,5,5-d₄. (R)-3-Ethynyl-3-hydroxy-1-methylpyrrolidin-2-one-4,4,5,5-d₄ was then slurried with MTBE (60 mL) and the solids were filtered off and collected. The filter cake was washed with MTBE and the filtrate was concentrated to afford 6.46 g (79%) of (R)-3-ethynyl-3-hydroxy-1-methylpyrrolidin-2-one-4,4,5,5-d₄ as white solid. LC-MS (ESI): Mass calcd. for C₇H₅D₄NO₂ 143.1 m/z found 144 [M+H]⁺.

### Intermediate 2: (R)-3-Ethynyl-4,4-difluoro-3-hydroxy-1-methylpyrrolidin-2-one

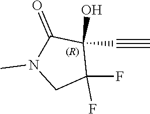

Step A. (3E)-1-Methyl-3-[(1-phenylethyl)imino]pyrrolidin-2-one. Into a 20 L 4-necked round-bottom flask was added 1-methylpyrrolidine-2,3-dione (346 g, 3060 mmol), MgSO₄ (368 g, 3060 mmol) and DCM (7 L). To this was added (+/−)-α-methylbenzylamine (389.21 g, 3211.74 mmol) and TFA (0.2 mL). The resulting mixture was stirred overnight at 40° C. under a nitrogen atmosphere. The mixture was allowed to cool to room temperature. The solids were filtered out and washed with DCM (1×1 L). The filtrate was collected and concentrated under reduced pressure. The product was slurried with diethyl ether (1.5 L). The solids were collected by filtration to afford (3E)-1-methyl-3-[(1-phenylethyl)imino]pyrrolidin-2-one (540 g) as a purple solid. ¹H NMR (300 MHz, DMSO-d₆) δ 7.39-7.08 (m, 5H), 5.41 (d, J=7.0 Hz, 1H), 4.88 (t, J=2.4 Hz, 1H), 4.30-4.13 (m, 1H), 3.76-3.52 (m, 2H), 2.89 (s, 3H), 1.40 (d, J=6.8 Hz, 3H).

Step B. 4,4-Difluoro-3,3-dihydroxy-1-methylpyrrolidin-2-one. Into a 10 L 4-necked round-bottom flask were added (3E)-1-methyl-3-[(1-phenylethyl)imino]pyrrolidin-2-one (540 g, 2497 mmol), Na₂SO₄ (511 g, 3600 mmol) and acetonitrile (5.4 L). To this was added Select-F (1946 g, 5493 mmol) in portions at 0° C. The reaction mixture was stirred overnight at room temperature and then acetonitrile was added (5.4 L). To the mixture was added HCl in 1,4-dioxane (4 N, 936 mL) dropwise at 0° C. The resulting mixture was stirred for an additional 1 h at room temperature. The resulting precipitate was filtered and washed with acetonitrile (1×2 L). The filtrate was concentrated under vacuum. This filtration and concentration were repeated 2 times again. The residue was purified by DAC using a C18 column CH₃CN/H₂O(0.1% NH₄HCO₃)=1% to 15% in 17 min to afford 4,4-difluoro-3,3-dihydroxy-1-methylpyrrolidin-2-one (122 g, 23.9% Steps A and B) as an off-white solid. ¹H NMR (300 MHz, DMSO-d₆) δ 7.29 (s, 2H), 3.65 (t, J=12.0 Hz, 2H), 2.80 (s, 3H).

Step C. (R)-3-Ethynyl-4,4-difluoro-3-hydroxy-1-methylpyrrolidin-2-one. Into a 5 L 4-necked round-bottom flask were added 4,4-difluoro-3,3-dihydroxy-1-methylpyrrolidin-2-one (122 g, 730 mmol), DMF (2440 mL), K₂CO₃ (10.09 g, 73.00 mmol), trimethylsilylacetylene (143 g, 1460 mmol) and Cu(OAc)₂ (26.52 g, 146.0 mmol) at room temperature under a N₂ atmosphere. The resulting mixture was stirred overnight at 50° C. The mixture was allowed to cool down to room temperature, quenched by the addition of MeOH (500 mL) at room temperature and concentrated under reduced pressure. The residue was purified by silica gel column chromatography, eluted with PE/EA (10:1 to 1:1) to afford (R,S)-3-ethynyl-4,4-difluoro-3-hydroxy-1-methylpyrrolidin-2-one (97 g, 75%) as a yellow solid. The (R) and (S) enantiomers of 3-ethynyl-4,4-difluoro-3-hydroxy-1-methylpyrrolidin-2-one (97 g and 6.2 g from a separate synthesis) were separated by Chiral-Prep-SFC (CHIRALPAK® IC-3 (50×4.6 mm), 80% Hexane with 0.1% DEA: 20% EtOH) and provided as the first eluting enantiomer (R)-3-ethynyl-4,4-difluoro-3-hydroxy-1-methylpyrrolidin-2-one (45.6 g, 88%) as an off-white solid. LC-MS (ESI): Mass calcd. for C₇H₇F₂NO₂ 175.0 m/z found 176 [M+H]⁺. ¹H NMR (300 MHz, DMSO-d₆) δ 7.43 (s, 1H), 3.84-3.80 (m, 3H), 2.82 (s, 3H).

### Intermediate 3: (3R,5S)-3-Ethynyl-3-hydroxy-1-methyl-5-(trifluoromethyl)pyrrolidin-2-one

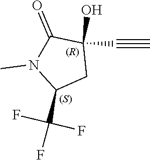

Step A. Ethyl 4,4,4-trifluoro-3-(methylamino)butanoate. To a solution of ethyl (E)-4,4,4-trifluorobut-2-enoate (4400 g, 26.17 mol, 3.89 L) in THF (18.0 L), was added MeNH₂ (3330 g, 32.17 mol, 30% purity). The light yellow solution was stirred at 25° C. for 3 h. The reaction mixture was concentrated under reduced pressure to provide the product as a yellow liquid (4.64 kg, 89%) which was used to next step without further purification. ¹H NMR (400 MHz, chloroform-d) δ 4.08-4.26 (m, 2H), 3.40-3.56 (m, 1H), 2.61-2.68 (m, 1H), 2.51-2.55 (m, 3H), 2.41-2.50 (m, 1H), 1.22-1.29 (m, 3H).

Step B. Ethyl 4-hydroxy-1-methyl-5-oxo-2-(trifluoromethyl)-2,5-dihydro-1H-pyrrole-3-carboxylate. To a solution of ethyl 4,4,4-trifluoro-3-(methylamino)butanoate (2400 g, 12.05 mol) in 2-MeTHF (24000 mL) was added t-BuOK (1.35 kg, 12.05 mol) and diethyl oxalate (1.76 kg, 12.05 mol, 1.65 L) at 25° C. under N₂ atmosphere. The reaction mixture was heated at 60° C. for 3 h under a N₂ atmosphere. The reaction mixture was quenched by the addition of NH₄Cl (saturated, 10.0 L) at 25° C., the pH of the mixture was adjusted to pH=2-3 with 1 M HCl (9.00 L), and the resulting mixture was extracted with EtOAc (5.00 L) twice. The combined organic extracts were washed with brine (15.0 L), dried over anhydrous Na₂SO₄, filtered and concentrated under reduced pressure to give a residue. The residue was purified by column chromatography (DCM/MeOH 0% to 3%) to afford the title compound as a brown oil (4.20 kg, 16.59 mol, 68.8%). ¹H NMR (400 MHz, chloroform-d) δ 4.63 (q, J=5.0 Hz, 1H), 4.24-4.42 (m, 4H), 3.10-3.14 (m, 3H), 1.19-1.26 (m, 2H).

Step C. 1-Methyl-5-(trifluoromethyl)pyrrolidine-2,3-dione. A mixture of ethyl 4-hydroxy-1-methyl-5-oxo-2-(trifluoromethyl)-2,5-dihydro-1H-pyrrole-3-carboxylate (2000 g, 7.90 mol) in HCl (10.96 kg, 108.23 mol, 10.75 L, 36% solution) was heated at 110° C. for 16 h. The reaction mixture was cooled to 20° C. and extracted with isopropylacetate (5000 mL×8). The combined organic extracts were washed with brine (10000 mL), dried over anhydrous Na₂SO₄, filtered and concentrated under reduced pressure to give a residue. The residue was triturated with MTBE (5000 mL) at 25° C. for 3 h. The solid was filtrated and washed with MTBE (1000 mL) and dried under high vacuum to afford the title compound as a yellow solid (1.15 kg, 6.35 mol, 40.2%). ¹H NMR (400 MHz, chloroform-d) δ 4.28-4.42 (m, 1H), 3.28 (s, 3H), 2.87-3.01 (m, 1H), 2.71-2.83 (m, 1H).

Step D. 3-Hydroxy-1-methyl-5-(trifluoromethyl)-3-((trimethylsilyl)ethynyl)pyrrolidin-2-one. To a solution of trimethylsilylacetylene (1.21 kg, 12.3 mol, 1.71 L) in THF (5400 mL) was added dropwise, n-BuLi (2.5 M, 4.95 L) at −70 to −60° C. over 30 mins and the mixture was stirred at −70 to −60° C. for 1 hr. 1-Methyl-5-(trifluoromethyl)pyrrolidine-2,3-dione (1120 g, 6.18 mol) in THF (9000 mL) was added dropwise to the mixture at −70 to −60° C. over 30 mins. The black solution was stirred at −70 to −60° C. for 2 h. The reaction mixture was added into NH₄Cl (aqueous saturated, 10000 mL) drop wise at 0° C., adjusted to pH=5 with 2 M HCl (5000 mL) and extracted the aqueous solution with EtOAc (5000 mL×3). The combined organic extracts were washed with 5000 mL brine, dried over anhydrous Na₂SO₄ and evaporated to dryness. The title compound was obtained as a red solid (1.50 kg, 86%) and was used without further purification. ¹H NMR (400 MHz, chloroform-d) δ 4.08-4.16 (m, 1H), 2.96-3.06 (m, 3H), 2.72-2.82 (m, 1H), 2.22-2.35 (m, 1H), 0.08-0.21 (m, 8H).

Step E. (3R,5S)-3-Ethynyl-3-hydroxy-1-methyl-5-(trifluoromethyl)pyrrolidin-2-one. 3-Hydroxy-1-methyl-5-(trifluoromethyl)-3-((trimethylsilyl)ethynyl)pyrrolidin-2-one (1500 g, 5.37 mol) was dissolved in MeOH (10.0 L), then K₂CO₃ (742 g, 5.37 mol) was added. The black mixture was stirred at 25° C. for 1 hr. The reaction mixture was filtered and the filter cake was washed with CH₂Cl₂ (4000 mL) and poured into water (3000 mL). The organic phase was separated, the aqueous phase was extracted with CH₂Cl₂ (3000 mL×2), the combined organic extracts were washed with water (2000 mL) and brine (2000 mL), dried over anhydrous Na₂SO₄, and concentrated to dryness under reduced pressure to afford a mixture of (3R,5S)-3-ethynyl-3-hydroxy-1-methyl-5-(trifluoromethyl)pyrrolidin-2-one and (3S,5R)-3-ethynyl-3-hydroxy-1-methyl-5-(trifluoromethyl)pyrrolidin-2-one. This mixture was purified by column chromatography (PE:EA=10:1 to 3:1) (635 g, 55%, 97.8% purity). (3R,5S)-3-Ethynyl-3-hydroxy-1-methyl-5-(trifluoromethyl)pyrrolidin-2-one and (3S,5R)-3-ethynyl-3-hydroxy-1-methyl-5-(trifluoromethyl)pyrrolidin-2-one were then separated by SFC (column: DAICEL CHIRALPAK® AD (250 mm×50 mm, 10 μm); mobile phase: [0.1% NH₃·H₂O EtOH]; B %: 20%-20%, min) to afford the title compound, (3R,5S)-3-ethynyl-3-hydroxy-1-methyl-5-(trifluoromethyl)pyrrolidin-2-one, the first eluting peak, as a yellow solid (95.0 g, 45.7%). ¹H NMR (400 MHz, chloroform-d) δ 3.97-4.08 (m, 1H), 3.03 (d, J=1.0 Hz, 3H), 2.80 (dd, J=13.6, 7.5 Hz, 1H), 2.62 (s, 1H), 2.33 (dd, J=13.7, 7.2 Hz, 1H), 1.23-1.28 (m, 1H).

### Intermediate 4: (Z)-6-Bromo-N-hydroxypicolinimidoyl chloride

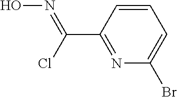 [SMILES: O/N=C(\Cl)c1cccc(Br)n1]

Step A. (E)-6-Bromopicolinaldehyde oxime. Into a 1 L 3-necked round-bottom flask was added 6-bromopyridine-2-carbaldehyde (40.0 g, 215 mmol), EtOH (600 mL) and NH₂OH·HCl (17.93 g, 258.1 mmol) at room temperature. To the above mixture was added sodium acetate (35.28 g, 430.1 mmol) in portions at 0° C. The resulting mixture was stirred overnight at room temperature and then concentrated under reduced pressure. The mixture was acidified to pH=7 with saturated NaHCO₃ (100 mL). The resulting mixture was extracted with EtOAc (2×100 mL). The combined organic extracts were washed with brine (1×150 mL) and dried over anhydrous Na₂SO₄. After filtration, the filtrate was concentrated under reduced pressure to a residue. The residue was purified by trituration with hexane (80 mL). The precipitated solids were collected by filtration and washed with hexane to afford the title compound as a white solid (39 g, 90%).

Step B. (Z)-6-Bromo-N-hydroxypicolinimidoyl chloride. Into a 2 L 4-necked round-bottom flask was added (E)-N-[(6-bromopyridin-2-yl)methylidene]hydroxylamine (39.0 g, 194 mmol) and DMF (1 L) at room temperature. To the above mixture was added NCS (31.09 g, 232.8 mmol) at room temperature. The resulting mixture was stirred overnight at room temperature. Then the pH of the mixture was adjusted to pH=7 with TFA (5 L, 1 M). The resulting mixture was extracted with MTBE (2×1 L). The combined organic extracts were washed with brine (1×1 L) and dried over anhydrous Na₂SO₄. After filtration, the filtrate was concentrated under reduced pressure to provide a residue. The residue was purified by trituration with hexane (80 mL). The precipitated solids were collected by filtration and washed with hexane to afford the title compound as a white solid (41.3 g, 90.2%). LC-MS (ESI): Mass calcd. for C₆H₄BrCIN₂O 233.9 m/z found 235 [M+H]⁺. ¹H NMR (300 MHz, DMSO-d₆) δ 12.89 (s, 1H), 7.91 (d, J=7.7, 1.2 Hz, 1H), 7.85 (t, J=7.6 Hz, 1H), 7.76 (d, J=7.6, 1.2 Hz, 1H).

### Intermediate 5: (Z)-6-Chloro-N-hydroxy-4-(trifluoromethyl)picolinimidoyl chloride

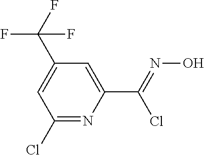 [SMILES: O/N=C(\Cl)c1cc(C(F)(F)F)cc(Cl)n1]

Step A. (E)-6-Chloro-4-(trifluoromethyl)picolinaldehyde oxime. In a 50 mL round-bottom flask charged with anhydrous ethanol (7.9 mL) was added hydroxylamine hydrochloride (182 mg, 2.63 mmol), sodium acetate (391 mg, 4.80 mmol), and 6-chloro-4-(trifluoromethyl)picolinaldehyde (500 mg, 2.4 mmol). The resulting mixture was stirred under nitrogen at room temperature for 12 hours. Water (50 mL) was then added followed by EtOAc (50 mL). The layers were separated and the aqueous layer was further extracted with EtOAc. The organic layers were combined, dried over anhydrous sodium sulfate and evaporated to dryness to provide the title compound as a white foam (600 mg). LC-MS (ESI): Mass calcd. for C₇H₄ClF₃N₂O 224.0 m/z found 225.0 [M+H]⁺.

Step B. (Z)-6-Chloro-N-hydroxy-4-(trifluoromethyl)picolinimidoyl chloride. To a 50 mL round-bottom flask was added DMF (anhydrous, 11 mL) followed by (E)-6-chloro-4-(trifluoromethyl)picolinaldehyde oxime (500 mg, 2.2 mmol), and NCS (387 mg, 2.90 mmol). The resulting solution was stirred for 12 hours at room temperature, after which time the solution was partitioned between EtOAc (30 mL) and water (30 mL). The aqueous portion was further extracted with EtOAc, the organic layers combined and dried over anhydrous sodium sulfate, filtered, and evaporated to dryness to yield a yellow foam. This residue was subjected to flash column chromatography, with an eluent from 0% to 100% EtOAc in hexanes, to yield the title compound as a yellow solid. LC-MS (ESI): Mass calcd. for C₇H₃Cl₂F₃N₂O 258.0 m/z found 258.9 [M+H]⁺.

### Intermediate 6: (R)-3-Hydroxy-1-methyl-3-(3-(6-(2-(methylsulfonyl)pyrimidin-4-yl)-4-(trifluoromethyl)pyridin-2-yl)isoxazol-5-yl)pyrrolidin-2-one

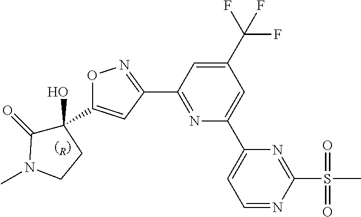

Step A. (R)-3-(3-(6-Chloro-4-(trifluoromethyl)pyridin-2-yl)isoxazol-5-yl)-3-hydroxy-1-methylpyrrolidin-2-one. To a 25 mL round-bottom flask was added EtOAc (11.2 mL), water (1.1 mL), NaHCO₃ (564 mg, 6.7 mmol), (R)-3-ethynyl-3-hydroxy-1-methylpyrrolidin-2-one (623 mg, 4.5 mmol), and (Z)-6-chloro-N-hydroxy-4-(trifluoromethyl)picolinimidoyl chloride (580 mg, 2.2 mmol). The resulting mixture was stirred at room temperature for 12 h. Then, the solution was partitioned between additional EtOAc (10 mL) and water (10 mL). The organic layer was dried over anhydrous sodium sulfate, filtered and evaporated to dryness to yield a yellow residue. This residue was subjected to flash column chromatography, with a gradient elution of 0% to 10% MeOH in DCM, to yield the title compound as a yellow solid (700 mg, 86%). LC-MS (ESI): Mass calcd. for C₁₄H₁₁ClF₃N₃O₃ 361.0 m/z found 362.0 [M+H]⁺.

Step B. (R)-3-Hydroxy-1-methyl-3-(3-(6-(2-(methylthio)pyrimidin-4-yl)-4-(trifluoromethyl)pyridin-2-yl)isoxazol-5-yl)pyrrolidin-2-one. A large microwave vial charged with DMF (9 mL, anhydrous) was sparged for 15 minutes under nitrogen before 2-(methylthio)-4-(tributylstannyl)pyrimidine (791 mg, 1.9 mmol), tetrakis(triphenylphosphine)-palladium(0) (200 mg, 0.173 mmol), and (R)-3-(3-(6-chloro-4-(trifluoromethyl)pyridin-2-yl)isoxazol-5-yl)-3-hydroxy-1-methylpyrrolidin-2-one (627 mg, 1.7 mmol) were added. The vial was sealed and subjected to microwave to irradiation at 140° C. for 90 minutes. After this time the resulting black solution was partitioned between EtOAc and water. The organic solution was dried over anhydrous sodium sulfate, filtered, and evaporated to dryness to yield a dark brown residue. This residue was subjected to flash column chromatography with 0% to 10% MeOH in DCM as eluent to afford the title compound as an orange foam (470 mg, 60%). LC-MS (ESI): Mass calcd. for C₁₉H₁₆F₃N₅O₃S 451.1 m/z found 452.2 [M+H]⁺.

Step C. (R)-3-Hydroxy-1-methyl-3-(3-(6-(2-(methylsulfonyl)pyrimidin-4-yl)-4-(trifluoromethyl)pyridin-2-yl)isoxazol-5-yl)pyrrolidin-2-one. (R)-3-Hydroxy-1-methyl-3-(3-(6-(2-(methylthio)pyrimidin-4-yl)-4-(trifluoromethyl)pyridin-2-yl)isoxazol-5-yl)pyrrolidin-2-one (470 mg, 1.0 mmol) was dissolved in acetone (2.2 mL), water (2.2 mL), THF (2.2 mL), and MeOH (2.2 mL). To this mixture was added potassium peroxymonosulfate (1.92 g, 3.10 mmol) and the resulting mixture was stirred for 12 hours at room temperature. To this mixture, ice water (20 mL) was added. After 10 minutes, the solids were removed by filtration, rinsed with additional water (100 mL), and dried for several hours under vacuum to yield the title compound as a white solid (300 mg, 60%). ¹H NMR (600 MHz, DMSO-d₆) δ 9.34 (d, J=5.1 Hz, 1H), 8.88 (d, J=5.1 Hz, 1H), 8.79 (d, J=1.4 Hz, 1H), 8.50 (d, J=1.4 Hz, 1H), 7.39 (s, 1H), 6.84 (s, 1H), 3.58 (s, 3H), 3.54-3.42 (m, 2H), 2.87 (s, 3H), 2.64 (ddd, J=13.0, 7.7, 5.0 Hz, 1H), 2.33 (ddd, J=13.5, 8.0, 5.5 Hz, 1H).

### Intermediate 7: (R)-4,4-Difluoro-3-hydroxy-1-methyl-3-(3-(6-(2-(methylsulfonyl)pyrimidin-4-yl)pyridin-2-yl)isoxazol-5-yl)pyrrolidin-2-one

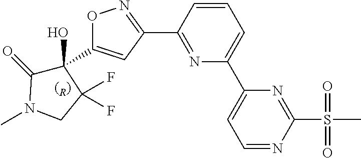

Step A. (R)-3-(3-(6-Bromopyridin-2-yl)isoxazol-5-yl)-4,4-difluoro-3-hydroxy-1-methylpyrrolidin-2-one. To a 25 mL round-bottom flask was added EtOAc (7.3 mL), water (0.73 mL), NaHCO₃ (642 mg, 6.6 mmol), (R)-3-ethynyl-4,4-difluoro-3-hydroxy-1-methylpyrrolidin-2-one (Intermediate 2, 535 mg, 3.1 mmol), and (Z)-6-bromo-N-hydroxypicolinimidoyl chloride (Intermediate 4, 600 mg, 2.6 mmol). This mixture was stirred at room temperature for 12 hours, after which time the solution was partitioned between EtOAc (10 mL) and water (10 mL). The organic portion was extracted, dried over anhydrous sodium sulfate, filtered and evaporated to dryness to yield a yellow residue, which was subjected to flash column chromatography, with 0% to 10% MeOH in DCM as eluent, to yield the title compound as a yellow solid. LC-MS (ESI): Mass calcd. for C₁₃H₁₀BrF₂N₃O₃ 373.0 m/z found 374.0 [M+H]⁺.

Step B. (R)-4,4-Difluoro-3-hydroxy-1-methyl-3-(3-(6-(2-(methylthio)pyrimidin-4-yl)pyridin-2-yl)isoxazol-5-yl)pyrrolidin-2-one. A large microwave vial charged with DMF (7.5 mL, anhydrous) was sparged under nitrogen for 15 minutes before addition of 2-(methylthio)-4-(tributylstannyl)pyrimidine (683 mg, 1.6 mmol), tetrakis(triphenylphosphine)-palladium(0) (173 mg, 0.15 mmol), and (R)-3-(3-(6-bromopyridin-2-yl)isoxazol-5-yl)-4,4-difluoro-3-hydroxy-1-methylpyrrolidin-2-one (560 mg, 1.5 mmol). The vial was sealed and subjected to microwave irradiation at 140° C. for 90 minutes. After this time the resulting black solution was partitioned between EtOAc and water and the aqueous portion was extracted with EtOAc. The organic portion was dried over anhydrous sodium sulfate, filtered, and evaporated to dryness to yield a dark brown residue. This residue was purified by flash column chromatography 0% to 10% MeOH in DCM to provide the title compound as an orange foam (650 mg). LC-MS (ESI): Mass calcd. for C₁₈H₁₅F₂N₅O₃S 419.1 m/z found 420.2 [M+H]⁺.

Step C. (R)-4,4-Difluoro-3-hydroxy-1-methyl-3-(3-(6-(2-(methylsulfonyl)pyrimidin-4-yl)pyridin-2-yl)isoxazol-5-yl)pyrrolidin-2-one. (R)-4,4-Difluoro-3-hydroxy-1-methyl-3-(3-(6-(2-(methylthio)pyrimidin-4-yl)pyridin-2-yl)isoxazol-5-yl)pyrrolidin-2-one (650 mg, 1.6 mmol) was dissolved in acetone (3.3 mL), water (3.3 mL), THF (3.3 mL) and MeOH (3.3 mL). Potassium peroxymonosulfate (2.86 g, 4.70 mmol) was added and the resulting clear mixture was stirred for 12 hours at room temperature. Ice water (20 mL) was then added to the flask and after 10 minutes the solid was filtered off, rinsed with additional water (100 mL), and dried for several hours under vacuum to yield the title compound as a white solid (540 mg, 77%). ¹H NMR (600 MHz, DMSO-d₆) δ 9.27 (d, J=5.2 Hz, 1H), 8.86 (d, J=5.2 Hz, 1H), 8.71-8.64 (m, 1H), 8.32-8.25 (m, 2H), 7.99 (s, 1H), 7.43 (s, 1H), 4.03 (t, J=12.2 Hz, 2H), 3.55 (s, 3H), 2.98 (s, 3H).

### Intermediate 8: (3R,5S)-3-Hydroxy-1-methyl-3-(3-(6-(2-(methylsulfonyl)pyrimidin-4-yl)pyridin-2-yl)isoxazol-5-yl)-5-(trifluoromethyl)pyrrolidin-2-one

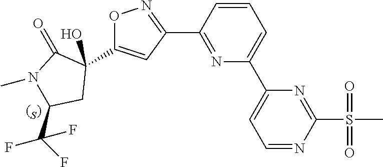

Step A. (3R,5S)-3-(3-(6-Bromopyridin-2-yl)isoxazol-5-yl)-3-hydroxy-1-methyl-5-(trifluoromethyl)pyrrolidin-2-one. To a 25 mL round-bottom flask was added EtOAc (4.2 mL), water (0.42 mL), NaHCO₃ (214 mg, 2.5 mmol), (3R,5S)-3-ethynyl-3-hydroxy-1-methyl-5-(trifluoromethyl)pyrrolidin-2-one (211 mg, 1.0 mmol, Intermediate 3), and (Z)-6-bromo-N-hydroxypicolinimidoyl chloride (Intermediate 4, 200 mg, 0.85 mmol). This mixture was stirred at room temperature for 12 hours. The solution was then partitioned between EtOAc (10 mL) and water (10 mL). The organic portion was extracted, dried over anhydrous sodium sulfate, filtered and evaporated to dryness to yield a yellow residue. This residue was purified by flash column chromatography (0% to 10% MeOH in DCM as the eluent) to yield the title compound as a yellow solid (360 mg). LC-MS (ESI): Mass calcd. for C₁₄H₁₁BrF₃N₃O₃ 405.0 m/z found 406.0 [M+H]⁺.

Step B. (3R,5S)-3-Hydroxy-1-methyl-3-(3-(6-(2-(methylthio)pyrimidin-4-yl)pyridin-2-yl)isoxazol-5-yl)-5-(trifluoromethyl)pyrrolidin-2-one. A 50 mL round-bottom flask was charged with DMF (21 mL, anhydrous) and was sparged under nitrogen for 15 minutes before 2-(methylthio)-4-(tributylstannyl)pyrimidine (1.2 g, 4.6 mmol), tetrakis(triphenylphosphine)-palladium(0) (484 mg, 0.42 mmol), and (3R,5S)-3-(3-(6-bromopyridin-2-yl)isoxazol-5-yl)-3-hydroxy-1-methyl-5-(trifluoromethyl)pyrrolidin-2-one (1.7 g, 1.5 mmol) were added. The vial was then sealed and subjected to microwave irradiation at 140° C. for 90 minutes. After this time the resulting black solution was partitioned between EtOAc and water and the aqueous portion extracted with EtOAc. The organic portion was dried over anhydrous sodium sulfate, filtered, and evaporated to dryness to yield a dark brown residue. This residue was subjected to flash column chromatography (0% to 10% MeOH in DCM as eluent) to yield the title compound as an orange foam (1.8 g).

Step C. (3R,5S)-3-Hydroxy-1-methyl-3-(3-(6-(2-(methylsulfonyl)pyrimidin-4-yl)pyridin-2-yl)isoxazol-5-yl)-5-(trifluoromethyl)pyrrolidin-2-one. (3R,5S)-3-Hydroxy-1-methyl-3-(3-(6-(2-(methylthio)pyrimidin-4-yl)pyridin-2-yl)isoxazol-5-yl)-5-(trifluoromethyl)pyrrolidin-2-one (1.8 g, 4.1 mmol) was placed in a 100 mL flask and dissolved in acetone (10.2 mL), water (10.2 mL), THF (10.2 mL), and MeOH (10.2 mL). To this mixture was added potassium peroxymonosulfate (7.5 g, 12.3 mmol) and the resulting clear mixture was stirred for 12 hours at room temperature. To the white reaction mixture was added ice water (100 mL). After 10 minutes the solid was filtered, rinsed with additional water (100 mL), and dried under reduced pressure to yield the title compound as a white solid that was used without further purification (2.8 g). ¹H NMR (600 MHz, DMSO-d₆) δ 9.29 (d, J=5.1 Hz, 1H), 8.81 (d, J=5.1 Hz, 1H), 8.69-8.61 (m, 1H), 8.28-8.27 (m, 1H), 7.36 (s, 1H), 7.15 (s, 1H), 4.70-4.57 (m, 1H), 3.56 (s, 3H), 3.07 (dd, J=14.6, 8.8 Hz, 1H), 2.96 (s, 3H), 2.35 (dd, J=14.7, 4.4 Hz, 1H). LC-MS (ESI): Mass calcd. for C₁₉H₁₆F₃N₅O₅S 483.1 m/z found 484.3 [M+H]⁺.

### Intermediate 9: (R)-3-Hydroxy-1-methyl-3-(3-(6-(2-(methylsulfonyl)pyrimidin-4-yl)pyridin-2-yl)isoxazol-5-yl)pyrrolidin-2-one-4,4,5,5-d₄

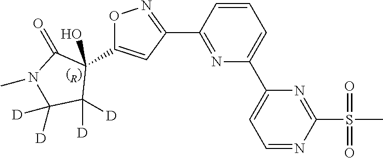

Step A. (R)-3-(3-(6-Bromopyridin-2-yl)isoxazol-5-yl)-3-hydroxy-1-methylpyrrolidin-2-one-4,4,5,5-d₄. The title compound (1.49 g, 102%) was prepared using conditions analogous to those described in Intermediate 8, Step A using (R)-3-ethynyl-3-hydroxy-1-methylpyrrolidin-2-one-4,4,5,5-d₄ (Intermediate 6) in place of (3R,5S)-3-ethynyl-3-hydroxy-1-methyl-5-(trifluoromethyl)pyrrolidin-2-one. LC-MS (ESI): Mass calcd. for C₁₃H₈D₄BrN₃O₃ 341.0 m/z found 342.1 [M+H]⁺.

Step B. (R)-3-Hydroxy-1-methyl-3-(3-(6-(2-(methylthio)pyrimidin-4-yl)pyridin-2-yl)isoxazol-5-yl)pyrrolidin-2-one-4,4,5,5-d₄. The title compound (1.6 g, 100%) was prepared with analogous conditions to those described in Intermediate 8, Step B using (R)-3-(3-(6-bromopyridin-2-yl)isoxazol-5-yl)-3-hydroxy-1-methylpyrrolidin-2-one-4,4,5,5-d₄ in place of (3R,5S)-3-(3-(6-bromopyridin-2-yl)isoxazol-5-yl)-3-hydroxy-1-methyl-5-(trifluoromethyl)pyrrolidin-2-one. LC-MS (ESI): Mass calcd. for C₁₈H₁₃D₄N₅O₃S 341.0 m/z found 342.1 [M+H]⁺.

Step C. (R)-3-Hydroxy-1-methyl-3-(3-(6-(2-(methylsulfonyl)pyrimidin-4-yl)pyridin-2-yl)isoxazol-5-yl)pyrrolidin-2-one-4,4,5,5-d₄. The title compound (1.9 g) was prepared with analogous conditions to those described in Intermediate 8, Step C using (R)-3-hydroxy-1-methyl-3-(3-(6-(2-(methylthio)pyrimidin-4-yl)pyridin-2-yl)isoxazol-5-yl)pyrrolidin-2-one-4,4,5,5-d₄ in place of (3R,5S)-3-hydroxy-1-methyl-3-(3-(6-(2-(methylthio)pyrimidin-4-yl)pyridin-2-yl)isoxazol-5-yl)-5-(trifluoromethyl)pyrrolidin-2-one. LC-MS (ESI): Mass calcd. for C₁₈H₁₃D₄N₅O₅S 419.6 m/z found 420.3 [M+H]⁺.

### Intermediate 10: N-(1-Methyl-1H-pyrazol-3-yl)-4-(trimethylstannyl)pyrimidin-2-amine

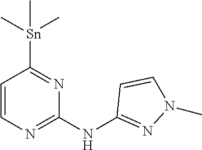 [SMILES: Cn1ccc(Nc2ncc[c]([Sn]([CH3])([CH3])[CH3])n2)n1]

A mixture of 4-chloro-N-(1-methyl-1H-pyrazol-3-yl)pyrimidin-2-amine (7.50 g, 35.8 mmol, Example 25 Step B), tetrakis(triphenylphosphine)palladium(0) (4.13 g, 3.58 mmol), hexamethylditin (17.2 g, 52.5 mmol) and 1,4-dioxane (250 mL) was heated at 100° C. for 16 h. The mixture was cooled to rt, diluted with aqueous KF (2 M, 150 mL), and stirred for 1 h. The mixture was then extracted with ethyl acetate, washed with brine, dried over anhydrous Na₂SO₄, filtered, and concentrated under reduced pressure to afford N-(1-methyl-1H-pyrazol-3-yl)-4-(trimethylstannyl)pyrimidin-2-amine (15.3 g, about 38% purity, 48%) as a brown powder which was used without further purification. LC-MS (ESI): Mass calcd. for C₁₁H₁₇N₅Sn 339.1 m/z found 339.5 [M+]⁺.

### Intermediate 11: (R)-3-Hydroxy-1-methyl-3-(3-(3-(4,4,5,5-tetramethyl-1,3,2-dioxaborolan-2-yl)phenyl)isoxazol-5-yl)pyrrolidin-2-one

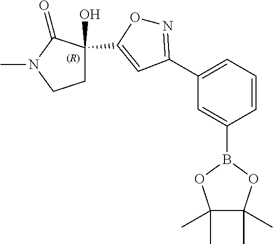

Step A: 3-Bromobenzaldehyde oxime. To a solution of 3-bromobenzaldehyde (3.0 g, 16 mmol) in EtOH (40 mL) was added NH₂OH (50% in water, 1.1 mL, 19 mmol). The reaction was stirred at 25° C. for 4 h. The reaction mixture was then concentrated under reduced pressure to afford 3-bromobenzaldehyde oxime (3.2 g, 98%) as a white solid. MS (ESI): Mass calcd. for C₇H₆BrNO, 199.0; m/z found, 200.1 [M+H]⁺.

Step B: 3-Bromo-N-hydroxybenzimidoyl chloride. To a solution of 3-bromobenzaldehyde oxime (3.20 g, 16.0 mmol) in anhydrous DCM (20 mL) was added N-chlorosuccinimide (2.56 g, 19.2 mmol), portion wise. The reaction mixture was stirred at 25° C. for 2 h and filtered. The filtrate was concentrated to afford 3-bromo-N-hydroxybenzimidoyl chloride (3.0 g, 95%) as yellow solid. MS (ESI): Mass calcd. for C₇H₅BrClNO, 234.5; m/z found, 235.2 [M+H]⁺.

Step C: (R)-3-(3-(3-Bromophenyl)isoxazol-5-yl)-3-hydroxy-1-methylpyrrolidin-2-one. To a solution of 3-bromo-N-hydroxybenzimidoyl chloride (1.50 g, 3.20 mmol) in DCM (20 mL) was added (R)-3-ethynyl-3-hydroxy-1-methylpyrrolidin-2-one (4.45 g, 3.20 mmol) followed by TEA (1.24 mL, 9.60 mmol). The reaction mixture was stirred at 25° C. for 16 h, diluted with DCM (30 mL), washed with brine (20 mL), dried over anhydrous Na₂SO₄, filtered and concentrated under reduced pressure. The residue was purified by preparative TLC (petroleum ether/ethyl acetate=1/1) to afford (R)-3-(3-(3-bromophenyl)isoxazol-5-yl)-3-hydroxy-1-methylpyrrolidin-2-one (0.6 g, 55%) as a yellow oil. MS (ESI): Mass calcd. for C₁₄H₁₃BrN₂O₃, 336.0; m/z found, 337.1 [M+H]⁺.

Step D: (R)-3-Hydroxy-1-methyl-3-(3-(3-(4,4,5,5-tetramethyl-1,3,2-dioxaborolan-2-yl)phenyl)isoxazol-5-yl)pyrrolidin-2-one. A flask containing (R)-3-(3-(3-bromophenyl)isoxazol-5-yl)-3-hydroxy-1-methylpyrrolidin-2-one (0.60 g, 1.8 mmol) in 1,4-dioxane (10 mL) was charged with bis(pinacolato)diboron (0.90 g, 3.56 mmol), Pd(dppf)Cl₂·CH₂Cl₂ (130 mg, 0.180 mmol) and KOAc (540 mg, 5.34 mmol). The reaction mixture was heated at 85° C. for 16 h under an Ar atmosphere. The mixture was concentrated and the residue was purified by preparative TLC (DCM/MeOH=20/1) to afford (R)-3-hydroxy-1-methyl-3-(3-(3-(4,4,5,5-tetramethyl-1,3,2-dioxaborolan-2-yl)phenyl)isoxazol-5-yl)pyrrolidin-2-one (0.62 g, 77%) as a brown solid. MS (ESI): Mass calcd. for C₂₀H₂₅BN₂O₅, 384.2; m/z found, 385.2 [M+H]⁺.

### Intermediate 12. (R)-3-(3-(3-(2-Chloropyrimidin-4-yl)phenyl)isoxazol-5-yl)-3-hydroxy-1-methylpyrrolidin-2-one

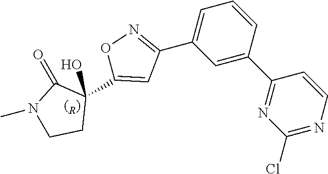

To a 100 mL round bottom flask was added, (R)-3-hydroxy-1-methyl-3-(3-(3-(4,4,5,5-tetramethyl-1,3,2-dioxaborolan-2-yl)phenyl)isoxazol-5-yl)pyrrolidin-2-one (Intermediate 11, 1.5 g, 3.9 mmol), 4-bromo-2-chloropyrimidine (906 mg, 4.69 mmol), K₂CO₃ (9.76 mL, 1 M, 9.76 mmol), PdCl₂(dppf) (143 mg, 0.195 mmol) and 1,4-dioxane (30 mL) under N₂. The reaction was heated at 50° C. for 1 hour. After the reaction mixture was cooled to room temperature, the reaction mixture was extracted from brine (sat. NaCl in water) (100 mL) with ethyl acetate (3×100 mL). The organic extract was dried over anhydrous MgSO₄, filtered, and concentrated to dryness under reduced pressure. The product was purified by chromatography using 0-100% ethyl acetate in hexane as eluent to yield (R)-3-(3-(3-(2-chloropyrimidin-4-yl)phenyl)isoxazol-5-yl)-3-hydroxy-1-methylpyrrolidin-2-one (1.522 g). LCMS (ESI): Mass calcd. for C₁₈H₁₅ClN₄O₃, 370.8; m/z found, 372 [M+H]⁺. ¹H NMR (500 MHz, DMSO-d₆) δ 8.89 (d, J=5.1 Hz, 1H), 8.64 (s, 1H), 8.39-8.23 (m, 2H), 8.12 (d, J=7.7 Hz, 1H), 7.73 (t, J=7.8 Hz, 1H), 7.20 (s, 1H), 6.77 (s, 1H), 3.54-3.48 (m, 1H), 3.48-3.43 (m, 1H), 2.87 (s, 3H), 2.67-2.54 (m, 1H), 2.37-2.24 (m, 1H).

### Intermediate 13. (R)-3-Hydroxy-1-methyl-3-(3-(6-(2-(methylsulfonyl)pyrimidin-4-yl)pyridin-2-yl)isoxazol-5-yl)pyrrolidin-2-one

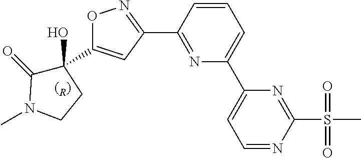

Step A. (R)-3-(3-(6-Bromopyridin-2-yl)isoxazol-5-yl)-3-hydroxy-1-methylpyrrolidin-2-one. To a solution of (R)-3-ethynyl-3-hydroxy-1-methylpyrrolidin-2-one (90.0 g, 646.78 mmol,) and (Z)-6-bromo-N-hydroxypicolinimidoyl chloride (Intermediate 4, 609 g, 2.59 mol) in EtOAc (900 mL) and H₂O (450 mL) was added NaHCO₃ (326 g, 3.88 mol, in 150 mL H₂O). The mixture was stirred at 25° C. for 24 h and then filtered and concentrated under reduced pressure to give a residue. The residue was purified by column chromatography (petroleum ether/ethyl acetate=1/0 to 0/1). The product was triturated with EtOH (4000 mL) at 25° C. for 12 hr. The above procedure was repeated 4 times. This resulting product was triturated with petroleum ether: ethyl acetate=1:1 (2000 mL) at 25° C. for 12 h and repeated twice to afford the title compound as a white solid (490 g, 50.4%). LC-MS (ESI): Mass calcd. for C₁₃H₁₂BrN₃O₃, 337.01; m/z found, 340.0 [M+H]⁺. ¹H NMR (400 MHz, DMSO-d₆) δ 8.04 (d, J=7.5 Hz, 1H), 7.92 (t, J=7.8 Hz, 1H), 7.79 (d, J=7.9 Hz, 1H), 6.94 (s, 1H), 6.77 (s, 1H), 3.52-3.38 (m, 2H), 2.83 (s, 3H), 2.59 (ddd, J=4.9, 7.8, 13.2 Hz, 1H), 2.27 (ddd, J=5.8, 7.9, 13.5 Hz, 1H).

Step B. (R)-3-Hydroxy-1-methyl-3-(3-(6-(tributylstannyl)pyridin-2-yl)isoxazol-5-yl)pyrrolidin-2-one. A mixture of (R)-3-(3-(6-bromopyridin-2-yl)isoxazol-5-yl)-3-hydroxy-1-methylpyrrolidin-2-one (270 g, 798 mmol), (SnBu₃)₂ (926 g, 1.60 mol, 798 mL), LiCl (169 g, 3.99 mol), tricyclohexylphosphine (22.3 g, 79.8 mmol, 25.8 mL) and Pd(PPh₃)₂Cl₂ (28.0 g, 39.9 mmol) in 1,4-dioxane (2200 mL) was degassed and purged with N₂ for 3 times, and then the mixture was heated at 110° C. for 12 hr under a N₂ atmosphere. The residue was diluted with H₂O (500 mL) and extracted with EtOAc (3000 mL, 1000 mL×3). The combined organic layers were washed with brine (800 mL×1), dried over anhydrous Na₂SO₄, filtered and concentrated under reduced pressure to give a residue. The residue was purified by flash silica gel chromatography (eluent of 0˜50% ethyl acetate/petroleum ether gradient) to afford the title compound as a yellow oil (320 g, 49.1%). ¹H NMR (400 MHz, Chloroform-d) δ 7.85 (dd, J=0.9, 7.9 Hz, 1H), 7.56 (t, J=7.6 Hz, 1H), 7.42 (dd, J=0.9, 7.4 Hz, 1H), 7.00 (s, 1H), 3.79-3.71 (m, 1H), 3.70-3.59 (m, 1H), 3.46 (dt, J=2.1, 9.5 Hz, 1H), 2.99 (s, 3H), 2.77 (ddd, J=2.0, 7.0, 13.3 Hz, 1H), 2.48 (td, J=8.7, 13.2 Hz, 1H), 1.70-1.50 (m, 7H), 1.35 (qd, J=7.3, 14.7 Hz, 7H), 1.23-1.05 (m, 6H), 0.90 (t, J=7.3 Hz, 9H).

Step C. (R)-3-Hydroxy-1-methyl-3-(3-(6-(2-(methylthio)pyrimidin-4-yl)pyridin-2-yl)isoxazol-5-yl)pyrrolidin-2-one. A mixture of (R)-3-hydroxy-1-methyl-3-(3-(6-(tributylstannyl)pyridin-2-yl)isoxazol-5-yl)pyrrolidin-2-one (320 g, 583 mmol), 4-iodo-2-(methylthio)pyrimidine (161 g, 641 mmol) and Pd(PPh₃)₄ (23.6 g, 20.4 mmol) in DMF (3200 mL) was degassed and purged with N₂ for 3 times, and then the mixture was heated at 130° C. for 12 h under N₂ atmosphere. The reaction mixture was cooled to 25° C., then concentrated under reduced pressure to remove DMF and diluted with EtOAc (2000 mL). KF (67.81 g, 1.17 mol, 27.34 mL) in H₂O (1500 mL) was then added to the mixture and stirred at 25° C. for 0.5 hr. The mixture was filtered and the filtrate extracted with EtOAc (1500 mL×3). The combined organic layers were washed with brine (800 mL×1), dried over anhydrous Na₂SO₄, filtered and concentrated under reduced pressure to give a residue. The product was triturated with ethyl acetate:petroleum ether (1:2, 1200 mL) at 25° C. for 30 min to afford the title compound as a white solid (180 g, 53%). ¹H NMR (400 MHz, DMSO-d₆) δ 8.83 (d, J=5.1 Hz, 1H), 8.59-8.46 (m, 1H), 8.28-8.08 (m, 3H), 7.20 (s, 1H), 6.78 (s, 1H), 3.58-3.39 (m, 2H), 2.85 (s, 3H), 2.63 (s, 4H), 2.30 (ddd, J=5.7, 7.8, 13.4 Hz, 1H). LC-MS (ESI): Mass calcd. for C₁₈H₁₇N₅O₃S, 383.1; m/z found, 384.0 [M+H]⁺.

Step D. (R)-3-Hydroxy-1-methyl-3-(3-(6-(2-(methylsulfonyl)pyrimidin-4-yl)pyridin-2-yl)isoxazol-5-yl)pyrrolidin-2-one. To a solution of (R)-3-hydroxy-1-methyl-3-(3-(6-(2-(methylthio)pyrimidin-4-yl)pyridin-2-yl)isoxazol-5-yl)pyrrolidin-2-one (130 g, 339 mmol) in THF (260 mL), acetone (260 mL), H₂O (260 mL) and MeOH (260 mL) was added potassium peroxymonosulfate (416 g, 678 mmol). The mixture was stirred at 25° C. for 12 h. The reaction mixture was diluted with ice-cold H₂O (3000 mL). The resulting precipitate was collected by filtration and concentrated under reduced pressure to give a residue. This residue was triturated with H₂O (2000 mL) at 25° C. for 1.0 h. This trituration was repeated twice. The resulting residue was further triturated with MeOH (700 mL) at 25° C. for 0.5 h to afford the title compound was obtained as a white solid (190 g, 87.9%). ¹H NMR (400 MHz, DMSO-d₆) δ 9.27 (d, J=5.1 Hz, 1H), 8.81 (d, J=5.3 Hz, 1H), 8.68-8.60 (m, 1H), 8.26 (d, J=4.5 Hz, 2H), 7.27 (s, 1H), 6.79 (s, 1H), 3.55 (s, 3H), 3.53-3.42 (m, 2H), 2.85 (s, 3H), 2.62 (ddd, J=5.1, 7.7, 13.2 Hz, 1H), 2.31 (ddd, J=5.8, 7.8, 13.4 Hz, 1H). LC-MS (ESI): Mass calcd. for C₁₈H₁₇N₅O₅S, 415.1; m/z found, 416.0 [M+H]⁺.

### Intermediate 14. 3-Cyclopropoxy-1-(2,2-difluoroethyl)-1H-pyrazol-4-amine

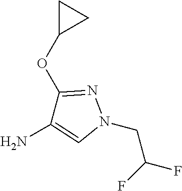 [SMILES: Nc1cn(CC(F)F)nc1OC1CC1]

Step A. 1,4-Dinitro-1H-pyrazole. KNO₃ (0.89 g, 8.84 mmol), 4-nitro-1H-pyrazole (1.00 g, 8.84 mmol), and anhydrous CH₂Cl₂ (5 mL) were added to a 50 mL round-bottomed flask. The mixture was cooled to 15° C., treated with a solution of 2,2,2-trifluoroacetic anhydride (2.46 mL, 17.7 mmol) and CH₂Cl₂ (5 mL), and stirred at room temperature for 5 h under N₂. After this time, the light yellow mixture was poured into ice water (20 mL) and extracted with ethyl acetate (20 mL×3). The combined organic extracts were washed with brine (50 mL), dried over anhydrous Na₂SO₄, filtered, and concentrated to dryness in vacuo to give a light yellow oil. The light yellow oil was purified by silica gel chromatography (0-20% EtOAc/pet ether) to afford 1,4-dinitro-1H-pyrazole as a yellow oil (1.2 g, 86%). ¹H NMR (400 MHz, CDCl₃) δ 8.97 (d, J=0.8 Hz, 1H), 8.15-8.09 (m, 1H).

Step B. 3-Cyclopropoxy-4-nitro-1H-pyrazole. NaH (1.35 g, 33.7 mmol, 60 wt. % in mineral oil) was added in portions to a 100 mL three-necked round-bottomed flask containing cyclopropanol (1.96 g, 33.7 mmol), and 2-MeTHF (15 mL) at 0° C. The reaction was stirred at room temperature for 10 min, and then added dropwise to another solution consisting of 1,4-dinitro-1H-pyrazole (8.00 g, 50.6 mmol) and 2-MeTHF (15 mL) at −78° C. The resulting mixture was stirred at −78° C. for 1 h and then allowed to warm to r.t. and stirred for 5 h. After this time, the grey mixture was poured into water, treated with aq. HCl (10 mL, 3.0 N) and extracted with CH₂Cl₂ (20 mL×3). The combined organic extracts were washed with brine (50 mL), dried over anhydrous Na₂SO₄, filtered, and concentrated to dryness in vacuo to give a yellow oil. This oil was purified by silica gel chromatography (0-30% EtOAc/pet ether) to afford 3-cyclopropoxy-4-nitro-1H-pyrazole as a yellow oil (1.1 g, 19%). ¹H NMR (400 MHz, CDCl₃) δ 8.30-8.17 (m, 1H), 6.47 (s, 1H), 4.27-4.23 (m, 1H), 1.03-0.94 (m, 2H), 0.88-0.79 (m, 2H).

Step C. 3-Cyclopropoxy-1-(2,2-difluoroethyl)-4-nitro-1H-pyrazole. 2-(Tributylphosphoranylidene)acetonitrile (3.00 g, 12.4 mmol) was added to a 100 mL three-necked round-bottomed flask containing a solution of 3-cyclopropoxy-4-nitro-1H-pyrazole (700 mg, 4.14 mmol), 2,2-difluoroethanol (1.02 g, 12.4 mmol), and anhydrous toluene (30 mL) at room temperature under N₂. The red mixture was heated at 60° C. for 12 h. The reaction vessel was removed from the oil bath and allowed to gradually cool to r.t. The mixture was concentrated to dryness in vacuo to give a red oil. The red oil was purified by silica gel chromatography (20-50% EtOAc/pet ether) to give 3-cyclopropoxy-1-(2,2-difluoroethyl)-4-nitro-1H-pyrazole as a red solid (500 mg, 52%). ¹H NMR (400 MHz, CDCl₃) δ 8.10 (s, 1H), 6.34-5.94 (m, 1H), 4.38-4.30 (m, 2H), 4.22-4.19 (m, 1H), 0.96-0.90 (m, 2H), 0.84-0.78 (m, 2H).

Step D. 3-Cyclopropoxy-1-(2,2-difluoroethyl)pyrazol-4-amine. Into a 250 mL 3-necked round-bottom flask were added 3-cyclopropoxy-1-(2,2-difluoroethyl)-4-nitro-1H-pyrazole (9 g, 38.59 mmol,), EtOH (129 mL), H₂O (26 mL), Fe (10.78 g, 192.99 mmol,) and NH₄Cl (8.26 g, 154.39 mmol) at room temperature. The resulting mixture was heated for 3 h at 70° C. The resulting mixture was filtered and the filter cake was washed with MeOH (1×250 mL). The filtrate was concentrated under reduced pressure. The resulting residue was diluted with water (100 mL) and extracted with EtOAc (3×200 mL). The combined organic layers were washed with water (3×500 mL) and brine (3×500 mL), dried over anhydrous Na₂SO₄, filtered and concentrated under reduced pressure. The residue was purified by silica gel column chromatography (eluted with PE/EA (1:1)) to afford 3-cyclopropoxy-1-(2,2-difluoroethyl)pyrazol-4-amine (3.124 g, 40%) as a brown oil. ¹H NMR (300 MHz, DMSO-d₆) δ 6.99 (s, 1H), 6.18 (tt, J=55.4, 4.0 Hz, 1H), 4.27 (td, J=14.8, 4.0 Hz, 2H), 4.00 (tt, J=6.0, 3.3 Hz, 1H), 3.47 (s, 2H), 0.64 (dddd, J=9.6, 5.7, 2.3, 1.2 Hz, 4H). ¹⁹F NMR (282 MHz, DMSO-d₆, ppm) δ −122.30. LC-MS (ESI): Mass calcd. for C₈H₁₁F₂N₃O, 203.1; m/z found, 204.0 [M+H]⁺.

### Intermediate 15. 2-(4-Amino-3-methoxypyrazol-1-yl)ethanol

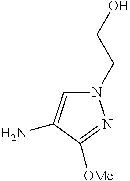 [SMILES: COc1nn(CCO)cc1N]

Step A. 1-{2-[(tert-Butyldimethylsilyl)oxy]ethyl}-3-methoxy-4-nitropyrazole. To a stirred solution of 3-methoxy-4-nitro-1H-pyrazole (10.00 g, 69.88 mmol), 2-[(tert-butyldimethylsilyl)oxy]ethanol (30.81 g, 174.7 mmol) and triphenylphosphine (45.82 g, 174.7 mmol) in tetrahydrofuran (100 mL) was added DIAD (22.87 g, 174.7 mmol) dropwise at 0° C. under a nitrogen atmosphere. The resulting mixture was stirred overnight at 60° C. under a nitrogen atmosphere. The reaction was quenched with water at room temperature. The resulting mixture was extracted with EtOAc (2×100 mL). The combined organic extracts were washed with brine (2×100 mL) and dried over anhydrous Na₂SO₄. After filtration, the filtrate was concentrated under reduced pressure. The residue was purified by silica gel column chromatography, eluted with PE/EA (10:1) to afford 1-{2-[(tert-butyldimethylsilyl)oxy]ethyl}-3-methoxy-4-nitropyrazole (11 g, 52%) as a light yellow oil.

Step B. 1-{2-[(tert-Butyldimethylsilyl)oxy]ethyl}-3-methoxypyrazol-4-amine. To a solution of 1-{2-[(tert-butyldimethylsilyl)oxy]ethyl}-3-methoxy-4-nitropyrazole (11 g, 36.49 mmol) in EtOH (250 mL) was added Pd/C (10%, 7.77 g) under a nitrogen atmosphere in a 500 mL round-bottom flask. The mixture was stirred overnight under an atmosphere of hydrogen using a hydrogen balloon, filtered through a pad of diatomaceous earth and the filtrate was concentrated under reduced pressure. The residue was purified by silica gel column chromatography, eluted with PE/EA (10:1) to afford 1-{2-[(tert-butyldimethylsilyl)oxy]ethyl}-3-methoxypyrazol-4-amine (8.7 g, 87.83%) as a brown oil. LCMS (ESI): Mass calcd. for C₁₂H₂₅N₃O₂Si, 271.2; m/z found, 272 [M+H]⁺.

Step C. 2-(4-Amino-3-methoxypyrazol-1-yl)ethanol. A solution of 1-{2-[(tert-butyldimethylsilyl)oxy]ethyl}-3-methoxypyrazol-4-amine (8.7 g, 32.05 mmol) and TBAF (10.06 g, 38.46 mmol) in tetrahydrofuran (87 mL) was stirred for 2 h at room temperature under a nitrogen atmosphere. The resulting mixture was concentrated under reduced pressure. The residue was purified by silica gel column chromatography, eluted with PE/EA (1:1) to afford 2-(4-amino-3-methoxypyrazol-1-yl)ethanol (2.258 g, 44.86%) as an off-white solid. LCMS (ESI): Mass calcd. for C₆H₁₁N₃O₂, 157.1; m/z found, 158 [M+H]⁺. ¹H NMR (300 MHz, DMSO-d₆) δ 6.94 (s, 1H), 4.74 (t, J=5.4 Hz, 1H), 3.80 (t, J=5.8 Hz, 2H), 3.75 (s, 3H), 3.60 (q, J=5.6 Hz, 2H), 3.31 (br, 2H).

### Intermediate 16. 3-Methoxy-1-(1-methylpiperidin-4-yl)pyrazol-4-amine

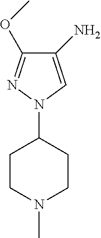

Step A. 4-(3-Methoxy-4-nitropyrazol-1-yl)-1-methylpiperidine. To a stirred solution of 3-methoxy-4-nitro-1H-pyrazole (5 g, 34.9 mmol), triphenylphosphine (13.75 g, 52.41 mmol) and 1-methyl-4-piperidinol (6.04 g, 52.4 mmol) in tetrahydrofuran (50 mL) was added DIAD (10.60 g, 52.42 mmol) dropwise at 0° C. under a nitrogen atmosphere. The resulting mixture was stirred for overnight at room temperature. The reaction was monitored by LCMS. The reaction was quenched by the addition of water/Ice (100 mL) at room temperature. The resulting mixture was extracted with CH₂Cl₂ (3×100 mL). The combined organic extracts were washed with brine (1×100 mL) and water (1×100 mL) and dried over anhydrous Na₂SO₄. After filtration, the filtrate was concentrated under reduced pressure. The residue was purified by silica gel column chromatography, eluted with CH₂Cl₂/MeOH (10:1) to afford 4-(3-methoxy-4-nitropyrazol-1-yl)-1-methylpiperidine (3.2 g, 38.12%) as a light yellow solid. LCMS (ESI): Mass calcd. for C₁₀H₁₆N₄O₃, 240.1; m/z found, 241 [M+H]⁺.

Step B. 3-Methoxy-1-(1-methylpiperidin-4-yl)pyrazol-4-amine. To a solution of 4-(3-methoxy-4-nitropyrazol-1-yl)-1-methylpiperidine (3.2 g, 13.31 mmol) in EtOH (40 mL) was added Pd/C (10%, 0.5 g) under nitrogen atmosphere in a 250 mL round-bottom flask. The mixture was stirred for 4 h under an atmosphere of hydrogen using hydrogen balloon, filtered through a pad of diatomaceous earth and the filtrate was concentrated under reduced pressure. This provided 3-methoxy-1-(1-methylpiperidin-4-yl)pyrazol-4-amine (2.25 g, 78%) as a brown oil. LCMS (ESI): Mass calcd. for C₁₀H₁₈N₄O, 210.2; m/z found, 210 [M]⁺. ¹H NMR (300 MHz, DMSO-d₆) δ 6.97 (s, 1H), 3.75 (s, 3H), 3.69 (dt, J=10.8, 4.6 Hz, 1H), 2.79 (dt, J=11.9, 3.2 Hz, 2H), 2.17 (s, 3H), 1.97 (td, J=11.6, 2.8 Hz, 2H), 1.90-1.69 (m, 4H).

### Intermediate 17. 3-(4-Amino-3-methyl-1H-pyrazol-1-yl)cyclobutan-1-ol

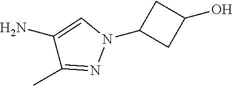

Step A. 3-(Benzyloxy)cyclobutan-1-ol. Into a 500 mL 3-necked round-bottom flask were added 3-(benzyloxy)cyclobutan-1-one (15 g, 85.12 mmol) and MeOH (150 mL) at room temperature under a N₂ atmosphere. To the above mixture was added NaBH₄ (3.2 g, 84.58 mmol) in portions at 0° C. The resulting mixture was stirred for additional 1 h at 0° C. and then concentrated under reduced pressure. The residue was diluted with EtOAc (100 mL), the organic layer was washed with brine (2×100 mL) and dried over anhydrous Na₂SO₄. After filtration, the filtrate was concentrated under reduced pressure to provide 3-(benzyloxy)cyclobutan-1-ol (12 g, 79%) as a yellow oil.

Step B. 3-(Benzyloxy)cyclobutyl 4-methylbenzenesulfonate. Into a 250 mL 3-necked round-bottom flask were added 3-(benzyloxy)cyclobutan-1-ol (12 g, 67.32 mmol), DCM (120 mL), DMAP (0.82 g, 6.71 mmol) and DIEA (13 g, 100.58 mmol) at room temperature under N₂ atmosphere. To the above mixture was added TsCl (15.3 g, 80.25 mmol) in portions at 0° C. The resulting mixture was stirred for overnight at room temperature. The resulting mixture was washed with H₂O (2×80 mL), dried with Na₂SO₄, filtered and concentrated under reduced pressure. This resulted in 3-(benzyloxy)cyclobutyl 4-methylbenzenesulfonate (16 g, 71.49%) as a yellow oil.

Step C. 1-[3-(Benzyloxy)cyclobutyl]-3-methyl-4-nitropyrazole. Into a 500 mL 3-necked round-bottom flask were added 3-(benzyloxy)cyclobutyl 4-methylbenzenesulfonate (16 g, 48.1 mmol), DMF (160 mL), 3-methyl-4-nitro-1H-pyrazole (4.7 g, 36.97 mmol) and Cs₂CO₃ (18 g, 55.24 mmol) at room temperature under a N₂ atmosphere. The resulting mixture was heated for 3 h at 100° C. and then diluted with H₂O (100 mL). The resulting mixture was extracted with EtOAc (3×100 mL), the combined organic extracts were washed with H₂O (3×100 mL) and brine (1×100 mL), and dried over anhydrous Na₂SO₄. After filtration, the filtrate was concentrated under reduced pressure. The residue was purified by silica gel column chromatography, eluted with PE/EA (10:1-3:1) to afford 1-[3-(benzyloxy)cyclobutyl]-3-methyl-4-nitropyrazole (9 g, 65%) as a yellow solid. LCMS (ESI): Mass calcd. for C₁₅H₁₇N₃O₃, 287.1; m/z found, 288 [M+H]⁺.

Step D. 3-(4-Amino-3-methyl-1H-pyrazol-1-yl)cyclobutan-1-ol. Into a 250 mL round-bottom flask were added 1-[3-(benzyloxy)cyclobutyl]-3-methyl-4-nitropyrazole (9 g, 31.32 mmol), EtOH (100 mL) and Pd/C (10%, 4 g) at room temperature. This mixture was degassed with H₂ three times. The resulting mixture was stirred under H₂ at 1 atmosphere overnight at room temperature, then filtered and the filtrate was concentrated under reduced pressure. The residue was purified by silica gel column chromatography, eluted with PE/EA (100:0-1:1) to afford the product. The product was further purified by SFC (Column: CHIRALPAK® IF, 3*25 cm, 5 μm; Mobile Phase A: CO₂, Mobile Phase B: MeOH (0.1% 2M NH₃-MeOH); Flow rate: 80 mL/min; Gradient: isocratic 30% B; Column Temperature (° C.): 35; Back Pressure (bar): 100; Wave Length: 220 nm; isolating the first peak at 3.5 minutes; Sample Solvent: MeOH-HPLC; Injection Volume: 3 mL;) to afford 3-(4-amino-3-methylpyrazol-1-yl)cyclobutan-1-ol (2.527 g, 48.25%) as a brown solid. LCMS (ESI): Mass calcd. for C₈H₁₃N₃O, 167.1; m/z found, 168 [M+H]⁺. ¹H NMR (300 MHz, DMSO-d₆) δ 6.99 (s, 1H), 5.09 (d, J=5.0 Hz, 1H), 4.72-4.54 (m, 1H), 4.44-4.26 (m, 1H), 3.55 (s, 2H), 2.59-2.41 (m, 2H), 2.30-2.13 (m, 2H), 1.99 (s, 3H).

### Intermediate 18. 3-Methyl-1-(oxan-4-yl)pyrazol-4-amine

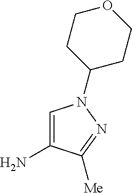 [SMILES: Cc1nn(C2CCOCC2)cc1N]

Step A. 3-Methyl-4-nitro-1-(oxan-4-yl)pyrazole. Into a 1 L 3-necked round-bottom flask were added 3-methyl-4-nitro-1H-pyrazole (15 g, 118.01 mmol), oxan-4-ol (30.13 g, 295.03 mmol), THF (150 mL) and PPh₃ (77.39 g, 295.03 mmol) at room temperature. To the above mixture was added DIAD (59.66 g, 295.03 mmol) dropwise at 0° C. The resulting mixture was heated overnight at 60° C. After the reaction mixture cooled to room temperature, it was diluted with ice/water (200 mL) and extracted with EA (3×100 mL). The combined organic extracts were washed with brine (1×100 mL), dried over anhydrous Na₂SO₄. After filtration, the filtrate was concentrated under reduced pressure. The residue was purified by silica gel column chromatography, eluted with PE/EA (5:1) to afford 3-methyl-4-nitro-1-(oxan-4-yl)pyrazole (12 g, 48%) as a black oil.

Step B. 3-Methyl-1-(oxan-4-yl)pyrazol-4-amine. To a solution of 3-methyl-4-nitro-1-(oxan-4-yl)pyrazole (12 g, 56.81 mmol) in EtOH (120 mL) was added Pd/C (10%, 7 g) under a nitrogen atmosphere in a 250 mL round-bottom flask. A hydrogen atmosphere was placed over the reaction mixture using a hydrogen balloon and the reaction was allowed to stir overnight. The reaction mixture was filtered through a pad of diatomaceous earth and concentrated under reduced pressure. The residue was purified by silica gel column chromatography, eluted with EA/MeOH (95:5) to afford the product. The product was further purified by SFC (SC 100×4.6 mm 3.0 m, Co Solvent B: 10% MeOH (20 mM NH₃), Peak 1.413 min, Total Flow: 3.0000 mL/min) to provide 3-methyl-1-(oxan-4-yl)pyrazol-4-amine (2.57 g, 24.96%) as a brown solid. LCMS (ESI): Mass calcd. for C₉H₁₅N₃O, 181; m/z found, 182.1 [M+H]⁺. ¹H NMR (300 MHz, DMSO-d₆) δ 7.01 (s, 1H), 4.08 (tt, J=10.6, 5.6 Hz, 1H), 3.98-3.85 (m, 2H), 3.50-3.33 (m, 2H), 3.34 (s, 2H), 1.99 (s, 3H), 1.91-1.71 (m, 4H).

### Intermediate 19. 1-(4-Amino-3-methoxy-1H-pyrazol-1-yl)-2-methylpropan-2-ol

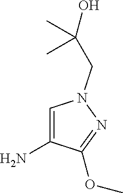

Step A. 1-(3-Methoxy-4-nitro-1H-pyrazol-1-yl)-2-methylpropan-2-ol. 3-Methoxy-4-nitro-1H-pyrazole (2.00 g, 14.0 mmol), DMF (30 mL), 2,2-dimethyloxirane (2.02 g, 28.0 mmol) and Cs₂CO₃ (13.7 g, 41.9 mmol) were added to a 100 mL round-bottom flask. The mixture was heated at 100° C. for 5 h. The mixture was diluted with water (50 mL) and extracted with EtOAc (3×50 mL). The combined organic extracts were washed with brine (100 mL), dried over anhydrous Na₂SO₄, and filtered. The filtrate was concentrated and give a white solid. The solid was purified by flash silica gel chromatography (Eluent of 0-30% EtOAc/pet ether gradient) to afford 1-(3-methoxy-4-nitro-1H-pyrazol-1-yl)-2-methylpropan-2-ol as a white solid (2.0 g, 63%). LCMS (ESI): Mass calcd. for C₈H₁₃N₃O₄, 215.1; m/z found, 216.1 [M+H]⁺.

Step B. 1-(4-Amino-3-methoxy-1H-pyrazol-1-yl)-2-methylpropan-2-ol. Pd/C (0.544 g, 10 wt. %, 0.511 mmol), 1-(3-methoxy-4-nitro-1H-pyrazol-1-yl)-2-methylpropan-2-ol (2.20 g, 10.2 mmol) and MeOH (30 mL) were added to a 100 mL round bottom flask, and the resulting mixture stirred under H₂ (15 psi) at room temperature for 3 h. The suspension was filtered through a pad of diatomaceous earth and the pad washed with EtOAc (100 mL) to give a nearly black viscous oil. The oil was purified by flash silica gel chromatography (Eluent of 0-50% EtOAc/pet ether gradient) to afford 1-(4-amino-3-methoxy-1H-pyrazol-1-yl)-2-methylpropan-2-ol as a blue syrupy solid (1850.5 mg, 96%). LCMS (ESI): Mass calcd. for C₈H₁₅N₃O₂, 185.1; m/z found, 186.1 [M+H]⁺. ¹H NMR (400 MHz, DMSO-d₆): 6.94 (s, 1H), 4.54 (s, 1H), 3.74 (s, 3H), 3.67 (s, 2H), 1.01 (s, 6H).

### Intermediate 20. 3-Methoxy-1-(oxetan-3-yl)-1H-pyrazol-4-amine

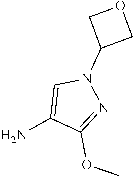

Step A. 3-Methoxy-4-nitro-1-(oxetan-3-yl)-1H-pyrazole. 3-Methoxy-4-nitro-1H-pyrazole (1.00 g, 6.99 mol), DMF (15 mL), 3-iodooxetane (2.57 g, 14.0 mol) and Cs₂CO₃ (6.83 g, 21.0 mol) were added to a 50 mL round-bottom flask. The mixture was heated at 100° C. for 5 h. The mixture was cooled to r.t. before it was diluted with water (30 mL) and extracted with EtOAc (3×40 mL). The combined organic extracts were washed with brine (30 mL), dried over anhydrous Na₂SO₄, filtered and concentrated in vacuo to give a yellow solid, which was purified by silica-gel chromatography (0-50% EtOAc/Petroleum ether) to give 3-methoxy-4-nitro-1-(oxetan-3-yl)-1H-pyrazole as a white solid (1.2 g, 62%). LCMS (ESI): Mass calcd. for C₇H₉N₃O₄, 199.1; m/z found, 200.2 [M+H]⁺.

Step B. 3-Methoxy-1-(oxetan-3-yl)-1H-pyrazol-4-amine. 3-Methoxy-4-nitro-1-(oxetan-3-yl)-1H-pyrazole (2.00 g, 10.0 mmol), wet Pd/C (1 g, 10% Pd, 50% water) and anhydrous EtOH (30 mL) were added to a 100 mL round-bottomed flask. The resulting mixture was sparged with H₂ for 3 times and then stirred at RT for 4 h under a H₂ atmosphere (15 Psi). The mixture was filtered and the filtrate was concentrated to dryness in vacuo to give a nearly black viscous oil, which was subjected to silica-gel chromatography (0-100% Ethyl acetate/Petroleum ether) to give 3-methoxy-1-(oxetan-3-yl)-1H-pyrazol-4-amine as a nearly black viscous oil (1 g, 56%). ¹H NMR (400 MHz, DMSO-d₆) δ 7.10-7.04 (m, 1H), 5.25-5.15 (m, 1H), 4.81-4.74 (m, 4H), 3.81 (s, 3H), 3.51-3.43 (m, 2H).

### Intermediate 21. (R)-3-Hydroxy-1-methyl-3-(5-(6-(2-(methylsulfonyl)pyrimidin-4-yl)pyridin-2-yl)isoxazol-3-yl)pyrrolidin-2-one

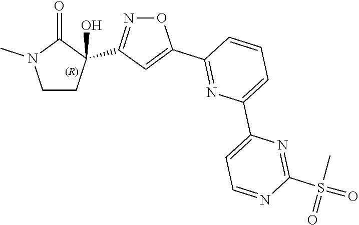

Step A. (R)-3-Ethynyl-1-methyl-3-((trimethylsilyl)oxy)pyrrolidin-2-one. To a solution of (R)-3-ethynyl-3-hydroxy-1-methylpyrrolidin-2-one (15.0 g, 107.8 mmol) and imidazole (22.0 g, 323.4 mmol) in DMF (50 mL) was added chlorotrimethylsilane (20.5 mL, 161.7 mmol) at rt. The reaction was heated to 60° C. and stirred overnight. The mixture was poured into a separatory funnel with a mixture of water (50 mL) and saturated aqueous NaHCO₃ (100 mL) and extracted with twice with DCM (1×100 mL and then 1×50 mL). The combined organic layers were washed with water (3×50 mL), dried (Na₂SO₄), filtered and concentrated. The product was purified by FCC (eluant: 10-70% EtOAc/hexanes) to give 18.788 g (82%) of (R)-3-ethynyl-1-methyl-3-((trimethylsilyl)oxy)pyrrolidin-2-one as a colorless oil that solidified upon standing. LCMS (ESI): mass calcd. for C₁₀H₁₇NO₂Si, 211.10; m/z found, 212.1 [M+1]⁺.

Step B. (R)-3-(3-(6-Chloropyridin-2-yl)-3-oxoprop-1-yn-1-yl)-1-methyl-3-((trimethylsilyl)oxy)pyrrolidin-2-one. To a solution of (R)-3-ethynyl-1-methyl-3-((trimethylsilyl)oxy)pyrrolidin-2-one (16.4 g, 77.6 mmol) in THF (275 mL) at −20° C. were added DIPEA (26.7 mL, 155.2 mmol), Pd(PPh₃)₄ (2.72 g, 5 mol %), and CuI (739 mg, 5 mol %). The mixture was stirred several minutes as the flask was purged with a stream of N₂. A solution of the acid chloride, 6-chloropicolinoyl chloride (19.1 g, 108.6 mmol), in THF (25 mL) was added via syringe at −20° C. The reaction was warmed to 0° C. and allowed to stir overnight. The reaction mixture was a stirred suspension of precipitate. Additional DIPEA (7 mL) was added, and the reaction mixture was passed through a 1 in. silica gel plug in a sintered glass funnel eluting with THF until the product was completely eluted from the plug. The product containing filtrate was concentrated causing precipitation of fine dark solids. The material was dissolved in EtOAc and filtered through a plug of diatomaceous earth. The filtrate containing the product was concentrated and purified by FCC (eluent: 30-70% EtOAc/hexanes) to give 4.72 g of a dark brown oil which was a mixture of the starting silyl-lactam and desired product in a 40:60 molar ratio. This mixture is 76 wt % (R)-3-(3-(6-chloropyridin-2-yl)-3-oxoprop-1-yn-1-yl)-1-methyl-3-((trimethylsilyl)oxy)pyrrolidin-2-one giving a yield of 3.59 g (13%) of desired product. The mixture was used as-is in the next step. LCMS (ESI): mass calcd. for C₁₆H₁₉ClN₂O₃Si, 350.08; m/z found, 351.1 [M+1]⁺.

Step C. (R)-3-(5-(6-Chloropyridin-2-yl)isoxazol-3-yl)-3-hydroxy-1-methylpyrrolidin-2-one. The mixture from the previous step containing (R)-3-(3-(6-chloropyridin-2-yl)-3-oxoprop-1-yn-1-yl)-1-methyl-3-((trimethylsilyl)oxy)pyrrolidin-2-one (3.59 g, 10.2 mmol) was stirred in THF (100 mL) at rt while NaN₃ (4.0 g, 61.4 mmol), AcOH (3.5 mL, 61.4 mmol), and Et₃N (0.57 mL, 4.1 mmol) were added. The mixture was allowed to stir overnight. A 1.0 M solution of TBAF in THF (30.7 mL, 30.7 mmol) was added, and the mixture was allowed to stir overnight to complete removal of the silyl group. The reaction mixture was poured into aqueous sodium carbonate (10.8 g Na₂CO₃, 102 mmol, in 110 mL water) and stirred 10 min. The mixture was extracted with EtOAc (3×100 mL). The combined organic layers were dried (Na₂SO₄), filtered and concentrated to give the product as a dark brown oil. The material was purified by FCC (eluant: 0-25% MeOH in 1:1 EtOAc/hexanes). The desired product was isolated in a 1.36 g mixture with the desilylated lactam starting material with an 83:17 molar ratio product/starting lactam. The product mixture was 91 wt % (R)-3-(5-(6-chloropyridin-2-yl)isoxazol-3-yl)-3-hydroxy-1-methylpyrrolidin-2-one giving a yield of 1.24 g (41%) of the desired product. LCMS (ESI): mass calcd. for C₁₃H₁₂ClN₃O₃, 293.06; m/z found, 294.1 [M+1]⁺.

Step D. (R)-3-Hydroxy-1-methyl-3-(5-(6-(2-(methylthio)pyrimidin-4-yl)pyridin-2-yl)isoxazol-3-yl)pyrrolidin-2-one. The mixture containing (R)-3-(5-(6-chloropyridin-2-yl)isoxazol-3-yl)-3-hydroxy-1-methylpyrrolidin-2-one (1.2 g, 4.1 mmol), Pd(PPh₃)₄ (330 mg, 7 mol %), and DMF (16 mL) were added to a 100 mL round-bottom flask. The flask was capped with a septum and flushed with N₂ for 15 min. The stannane, 2-(ethylthio)-4-(tributylstannyl)pyrimidine (1.53 mL, 4.5 mmol), was added, and the reaction was heated at 100° C. overnight. LCMS analysis showed near complete conversion of the starting pyridyl chloride. The reaction mixture was cooled, poured into a biphasic mixture of EtOAc (60 mL) and aqueous KF (1.2 g KF/60 mL water), and stirred for 30 min. The mixture was filtered through a plug of diatomaceous earth with excess EtOAc. The EtOAc was partially removed in vacuo, and the remaining biphasic mixture was poured in a separatory funnel and separated. The aqueous layer was extracted twice with EtOAc. The combined organic layers were dried (Na₂SO₄) and concentrated to give the product in residual DMF. The material was first purified by FCC (eluant: 0-10% MeOH/DCM), which gave partial separation. The mixed fractions were combined and again purified by FCC (eluent: 0-100% 5% MeOH in TBME/DCM). All pure fractions were combined to give (R)-3-hydroxy-1-methyl-3-(5-(6-(2-(methylthio)pyrimidin-4-yl)pyridin-2-yl)isoxazol-3-yl)pyrrolidin-2-one (0.65 g, 41%) as an off white solid. LCMS (ESI): mass calcd. for C₁₈H¹⁷N₅O₂S, 383.11; m/z found, 384.1 [M+1]⁺.

Step E. (R)-3-Hydroxy-1-methyl-3-(5-(6-(2-(methylsulfonyl)pyrimidin-4-yl)pyridin-2-yl)isoxazol-3-yl)pyrrolidin-2-one. (R)-3-Hydroxy-1-methyl-3-(5-(6-(2-(methylthio)pyrimidin-4-yl)pyridin-2-yl)isoxazol-3-yl)pyrrolidin-2-one (0.2 g, 0.52 mmol) was placed in a 25 mL flask and dissolved in acetone (1.7 mL), water (1.7 mL), THF (1.7 mL), and MeOH (1.7 mL). To this mixture was added potassium peroxymonosulfate (0.96 g, 1.6 mmol), and the resulting clear mixture was stirred 12 hours at room temperature. To the white reaction mixture was added ice water (10 mL) and left to stir 10 minutes before the solid was filtered, rinsed with additional water (20 mL), and dried under reduced pressure to yield a white solid that was used without further purification (0.16 g). LC-MS (ESI): Mass calcd. for C₁₈H₁₇N₅O₅S 415.1 m/z found 415.8 [M+H]⁺.

### Intermediate 22 and Intermediate 23. (R)-3-Hydroxy-1-methyl-3-(1-(6-(2-(methylsulfonyl)pyrimidin-4-yl)pyridin-2-yl)-1H-pyrazol-4-yl)pyrrolidin-2-one and (S)-3-hydroxy-1-methyl-3-(1-(6-(2-(methylsulfonyl)pyrimidin-4-yl)pyridin-2-yl)-1H-pyrazol-4-yl)pyrrolidin-2-one

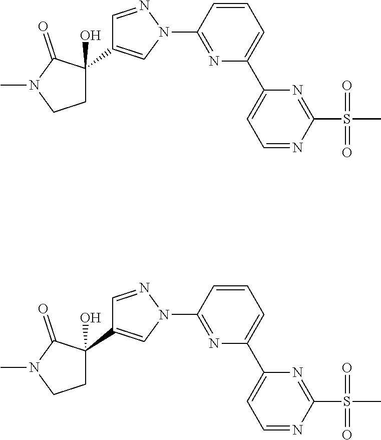

Step A. 2-Chloro-6-(4-iodo-1H-pyrazol-1-yl)pyridine. The below reaction was carried out in five batches. 4-Iodo-1H-pyrazole (200 g, 1.03 mol) and DMF (4000 mL) were added to a 5000 mL flask, which was charged with 2,6-dichloropyridine (381 g, 2.58 mol) and Cs₂CO₃ (1.01 kg, 3.09 mol). This mixture was heated at 100° C. for 3 h. The reaction mixture was filtered and the solids were washed with EtOAc. The filtrate was then diluted with H₂O (8000 mL) and extracted with MTBE (5000 mL×3). The organic layers were washed with brine (5000 mL×1), dried over Na₂SO₄, filtered and concentrated under reduced pressure to give a residue. The residue was triturated with petroleum ether:ethyl acetate=2:1 (2000 mL) at 25° C. for 1 h to provide 2-chloro-6-(4-iodo-1H-pyrazol-1-yl)pyridine (1.04 kg, 3.10 mol, 60%, 91.0% purity) as a white solid. LCMS (ESI): Mass calcd. for C₈H₅ClIN₃ 304.9 m/z, found 305.9 [M+H]⁺. ¹H NMR (400 MHz, DMSO-d₆) δ 8.67 (s, 1H), 8.06 (t, J=7.9 Hz, 1H), 7.99-7.95 (m, 1H), 7.87 (d, J=7.9 Hz, 1H), 7.50 (d, J=7.5 Hz, 1H).

Step B. 3-(1-(6-Chloropyridin-2-yl)-1H-pyrazol-4-yl)-3-hydroxy-1-methylpyrrolidin-2-one. To a solution of 2-chloro-6-(4-iodo-1H-pyrazol-1-yl)pyridine (1000 g, 3.27 mol,) in THF (15000 mL) was added iPrMgCl—LiCl (1.30 M, 2.64 L) at 0° C. under a N₂ atmosphere. The mixture was stirred at 0° C. for 0.5 h. 1-Methylpyrrolidine-2,3-dione (314 g, 2.78 mol) dissolved in THF (5000 mL) was added and the resulting mixture was stirred at 25° C. for another 12 h. The reaction mixture was quenched by the addition of NH₄Cl (saturated aqueous, 10 L) at 0° C. and extracted with EtOAc 15000 mL (5000 mL×3). The combined organic layers were washed with brine (5000 mL×2), dried over Na₂SO₄, filtered and concentrated under reduced pressure to give a residue. The residue was purified by column chromatography (SiO₂, petroleum ether/ethyl acetate=1/1 to 0/1) to provide 3-(1-(6-chloropyridin-2-yl)-1H-pyrazol-4-yl)-3-hydroxy-1-methylpyrrolidin-2-one (330 g, 1.07 mol, 32%, 95.0% purity) as a yellow solid. LCMS (ESI): Mass calcd. for C₁₃H₁₃ClN₄O₂ 292.1 m/z, found 293.0 [M+H]⁺. ¹H NMR (400 MHz, DMSO-d₆) δ 8.47 (s, 1H), 8.08-7.98 (m, 1H), 7.90-7.82 (m, 2H), 7.46 (d, J=7.8 Hz, 1H), 6.00 (s, 1H), 3.45-3.36 (m, 1H), 3.35-3.28 (m, 3H), 2.78 (s, 3H), 2.26-2.17 (m, 1H).

Step C. 3-Hydroxy-1-methyl-3-(1-(6-(tributylstannyl)pyridin-2-yl)-1H-pyrazol-4-yl)pyrrolidin-2-one. A mixture of 3-(1-(6-chloropyridin-2-yl)-1H-pyrazol-4-yl)-3-hydroxy-1-methylpyrrolidin-2-one (280 g, 956 mmol), (SnBu₃)₂ (1.11 kg, 1.91 mol, 956 mL), LiCl (202 g, 4.78 mol), Cy₃P (26.8 g, 95.6 mmol, 31.0 mL), Pd(PPh₃)₂Cl₂ (33.5 g, 47.8 mmol) in 1,4-dioxane (2500 mL) was degassed and purged with N₂ 3 times. The mixture was heated at 110° C. for 36 h under N₂ atmosphere. The reaction mixture was concentrated under reduced pressure to give a residue. The residue was diluted with H₂O (1000 mL) and extracted with EtOAc (1000 mL×3). The combined organic layers were washed with brine (800 mL), dried over Na₂SO₄, filtered and concentrated under reduced pressure to give a residue. The residue was purified by flash silica gel chromatography (Eluent of 0-46% ethyl acetate/petroleum ether gradient) to provide 3-hydroxy-1-methyl-3-(1-(6-(tributylstannyl)pyridin-2-yl)-1H-pyrazol-4-yl)pyrrolidin-2-one (225 g, 411 mmol, 42%) as a yellow oil. LCMS (ESI): Mass calcd. for C₂₅H₄₀N₄O₂Sn 548.1 m/z, found 549.2 [M+H]⁺. ¹H NMR: (400 MHz, DMSO-d₆) δ 8.57 (s, 1H), 7.76-7.68 (m, 2H), 7.61-7.52 (m, 1H), 7.27 (s, 1H), 3.41-3.25 (m, 3H), 2.88 (s, 3H), 2.54-2.40 (m, 2H), 1.63-1.46 (m, 7H), 1.37-1.23 (m, 7H), 1.14-1.03 (m, 6H), 0.85 (t, J=7.3 Hz, 10H).

Step D. (R) and (S) enantiomers of 3-Hydroxy-1-methyl-3-(1-(6-(2-(methylthio)pyrimidin-4-yl)pyridin-2-yl)-1H-pyrazol-4-yl)pyrrolidin-2-one. A mixture of 3-hydroxy-1-methyl-3-(1-(6-(tributylstannyl)pyridin-2-yl)-1H-pyrazol-4-yl)pyrrolidin-2-one (200 g, 365 mmol), 4-iodo-2-(methylthio)pyrimidine (101 g, 401 mmol) and Pd(PPh)₄ (14.7 g, 12.7 mmol, 0.035 eq) in DMF (1600 mL) was degassed and purged with N₂ 3 times, and then the mixture was heated at 120° C. for 12 h under N₂ atmosphere. The reaction mixture was cooled at 25° C., and then diluted with EtOAc (3000 mL). KF (42.2 g) in H₂O (800 mL) was added to the mixture and it was stirred at 25° C. for 0.5 h. The mixture was filtered and the filtrate extracted with EtOAc (1500 mL×3). The combined organic layers were washed with brine (800 mL×1), dried over Na₂SO₄, filtered and concentrated under reduced pressure to give a residue. This was then triturated with EtOAc (500 mL) at 25° C. for 60 min to provide (R,S)-3-hydroxy-1-methyl-3-(1-(6-(2-(methylthio)pyrimidin-4-yl)pyridin-2-yl)-1H-pyrazol-4-yl)pyrrolidin-2-one (90 g) as a yellow solid. The (R) and (S) enantiomers of (R,S)-3-hydroxy-1-methyl-3-(1-(6-(2-(methylthio)pyrimidin-4-yl)pyridin-2-yl)-1H-pyrazol-4-yl)pyrrolidin-2-one were separated by SFC (DAICEL CHIRALPAK AS (250 mm×50 mm, 10 m); mobile phase: 65% [0.1% NH₃H₂O in EtOH], 35% CO₂) to provide two eluting peaks. The second eluting peak was designated as enantiomer 1 of 3-hydroxy-1-methyl-3-(1-(6-(2-(methylthio)pyrimidin-4-yl)pyridin-2-yl)-1H-pyrazol-4-yl)pyrrolidin-2-one. LCMS (ESI): Mass calcd. for C₁₈H₁₈N₆O₂S 382.1 m/z, found 383.3 [M+H]⁺. ¹H NMR (400 MHz, DMSO-d₆) δ 8.88-8.80 (m, 2H), 8.36 (d, J=7.6 Hz, 1H), 8.23-8.20 (m, 1H), 8.19-8.16 (m, 1H), 8.07 (d, J=8.0 Hz, 1H), 7.88 (s, 1H), 6.01 (s, 1H), 3.42 (br dd, J=4.5, 8.1 Hz, 1H), 3.39-3.35 (m, 1H), 2.80 (s, 3H), 2.63 (s, 3H), 2.61-2.54 (m, 1H), 2.30-2.21 (m, 1H). The first eluting peak (retention time=2.25 min) was designated as enantiomer 2 of 3-hydroxy-1-methyl-3-(1-(6-(2-(methylthio)pyrimidin-4-yl)pyridin-2-yl)-1H-pyrazol-4-yl)pyrrolidin-2-one (39.0 g, 98.9 mmol, 97.0% purity) and was obtained as a white solid. LCMS (ESI): Mass calcd. for C₁₈H₁₈N₆O₂S 382.1 m/z, found 383.3 [M+H]⁺. ¹H NMR (400 MHz, DMSO-d₆) δ 8.88-8.80 (m, 2H), 8.36 (d, J=7.6 Hz, 1H), 8.23-8.20 (m, 1H), 8.19-8.16 (m, 1H), 8.07 (d, J=8.0 Hz, 1H), 7.88 (s, 1H), 6.01 (s, 1H), 3.42 (dd, J=4.5, 8.1 Hz, 1H), 3.39-3.35 (m, 1H), 2.80 (s, 3H), 2.63 (s, 3H), 2.61-2.54 (m, 1H), 2.30-2.21 (m, 1H).

Step E: To a solution of enantiomer 1 of 3-hydroxy-1-methyl-3-(1-(6-(2-(methylthio)pyrimidin-4-yl)pyridin-2-yl)-1H-pyrazol-4-yl)pyrrolidin-2-one (33.0 g, 86.2 mmol) in a mixture of THF (495 mL)/acetone (495 mL)/H₂O (495 mL) was added potassium peroxymonosulfate (159.14 g, 258 mmol). The mixture was stirred at 25° C. for 12 h. The reaction mixture was then diluted with H₂O (1500 mL), and then extracted with CH₂Cl₂ (1500 mL×3). The combined extracts washed with brine (1000 mL), dried over anhydrous Na₂SO₄, filtered and concentrated under reduced pressure to give a residue. This residue was triturated with H₂O (500 mL) at 25° C. for 1 h and then isolated. This isolated product was then triturated a second time with MeOH (500 mL) at 25° C. for 1 h to provide enantiomer 1 of 3-hydroxy-1-methyl-3-(1-(6-(2-(methylsulfonyl)pyrimidin-4-yl)pyridin-2-yl)-1H-pyrazol-4-yl)pyrrolidin-2-one (Intermediate 22, 29.0 g, 66.4 mmol, 77%, 95.0% purity) as a white solid. LCMS (ESI): Mass calcd. for C₁₈H₁₈N₆O₄S 414.1 m/z, found 415.3 [M+H]⁺. ¹H NMR (400 MHz, DMSO-d₆) δ 9.28 (d, J=5.1 Hz, 1H), 8.89 (s, 1H), 8.83 (d, J=5.3 Hz, 1H), 8.46 (d, J=7.5 Hz, 1H), 8.25 (t, J=7.9 Hz, 1H), 8.14 (d, J=8.1 Hz, 1H), 7.90 (s, 1H), 6.02 (br s, 1H), 3.55 (s, 3H), 3.48-3.41 (m, 1H), 3.40-3.35 (m, 1H), 2.80 (s, 3H), 2.66-2.53 (m, 1H), 2.36-2.21 (m, 1H).

Step F: Enantiomer 2 of 3-hydroxy-1-methyl-3-(1-(6-(2-(methylsulfonyl)pyrimidin-4-yl)pyridin-2-yl)-1H-pyrazol-4-yl)pyrrolidin-2-one (Intermediate 23) was made in a manner analogous to Intermediate 22 using enantiomer 2 of 3-hydroxy-1-methyl-3-(1-(6-(2-(methylthio)pyrimidin-4-yl)pyridin-2-yl)-1H-pyrazol-4-yl)pyrrolidin-2-one (prepared in Step D) in place of enantiomer 1 of 3-hydroxy-1-methyl-3-(1-(6-(2-(methylthio)pyrimidin-4-yl)pyridin-2-yl)-1H-pyrazol-4-yl)pyrrolidin-2-one. LCMS (ESI): Mass calcd. for C₁₈H₁₈N₆O₄S 414.1 m/z, found 415.3 [M+H]⁺. ¹H NMR (400 MHz, DMSO-d₆) δ 9.28 (d, J=5.3 Hz, 1H), 8.89 (s, 1H), 8.83 (d, J=5.1 Hz, 1H), 8.46 (d, J=7.5 Hz, 1H), 8.25 (t, J=7.9 Hz, 1H), 8.13 (d, J=8.3 Hz, 1H), 7.90 (s, 1H), 6.02 (s, 1H), 3.55 (s, 3H), 3.49-3.41 (m, 1H), 3.40-3.34 (m, 1H), 2.80 (s, 3H), 2.59 (ddd, J=4.6, 7.6, 12.7 Hz, 1H), 2.33-2.21 (m, 1H).

### Intermediate 24. N-(1-Methyl-1H-pyrazol-3-yl)-4-(tributylstannyl)pyrimidin-2-amine

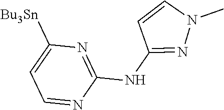

4-Chloro-N-(1-methyl-1H-pyrazol-3-yl)pyrimidin-2-amine (300 mg, 1.43 mmol, Example 25, Step B), LiCl (364 mg, 8.59 mmol), 1,1,1,2,2,2-hexabutyldistannane (1.79 g, 3.09 mmol), and 1,4-dioxane (10 mL) were added to a 50 mL flask fitted with a reflux condenser. The resulting mixture was purged with N₂ for three times and then treated with Pd₂(dba)₃ (66 mg, 0.072 mmol) and tricyclohexylphosphine (40 mg, 0.14 mmol). The resultant mixture was purged with N₂ for another three times and then heated at 120° C. for 16 hours. After this time, the reaction vessel was removed from the oil bath and allowed to gradually cool to r.t. The reaction mixture was quenched with sat. KF (30 mL), stirred for 1 h, and extracted with EtOAc (20 mL×3). The combined organic extracts were washed with brine (20 mL), dried over anhydrous Na₂SO₄, filtered, and concentrated to dryness in vacuo to give a brown solid. The solid was subjected to silica gel chromatography (0-30% EtOAc/pet ether) to give N-(1-methyl-1H-pyrazol-3-yl)-4-(tributylstannyl)pyrimidin-2-amine as a white solid (350 mg, 53%). MS (ESI⁺): m/z=466.0.

### Intermediate 25. 3-(2-(6-Chloropyridin-2-yl)thiazol-5-yl)-3-hydroxy-1-methylpyrrolidin-2-one

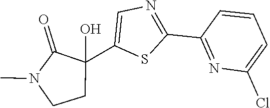 [SMILES: CN1CCC(O)(c2cnc(-c3cccc(Cl)n3)s2)C1=O]

Step A. 3-(2-Bromothiazol-5-yl)-3-hydroxy-1-methylpyrrolidin-2-one. 2,5-Dibromothiazole (2.8 g, 11.5 mmol) and anhydrous THF (30 mL) were added to a 100 mL three-necked round-bottomed flask and the mixture was cooled to −78° C. Then i-PrMgCl·LiCl (9.5 mL, 1.3 M in hexane, 12.4 mmol) was added dropwise. The resulting mixture was stirred at −78° C. for 30 min and then 1-methylpyrrolidine-2,3-dione (1.0 g, 8.8 mmol) in THF (10 mL) was added dropwise into the mixture. The resulting mixture was stirred for 16 h with gradual warming to room temperature. After this time, the mixture was poured into water (80 mL) and extracted with ethyl acetate (80 mL×3). The organic extracts were washed with brine (60 mL), dried with anhydrous Na₂SO₄, filtered, and concentrated to dryness in vacuo to give a brown oil. The oil was purified by silica gel chromatography (0-100% EtOAc/pet ether) to give 3-(2-bromothiazol-5-yl)-3-hydroxy-1-methylpyrrolidin-2-one as a yellow oil (560 mg, 19%). LCMS (ESI): Mass calcd. for C₈H₉BrN₂O₂S 276.0 m/z, found 276.9 [M+1]⁺.

Step B. 3-(2-(6-Chloropyridin-2-yl)thiazol-5-yl)-3-hydroxy-1-methylpyrrolidin-2-one. 3-(2-Bromothiazol-5-yl)-3-hydroxy-1-methylpyrrolidin-2-one (560 mg, 2.0 mmol), 1,4-dioxane (8 mL), H₂O (2 mL), 2-chloro-6-(4,4,5,5-tetramethyl-1,3,2-dioxaborolan-2-yl)pyridine (580 mg, 2.4 mmol), and K₂CO₃ (590 mg, 6.0 mmol) were added to a nitrogen-purged 10 mL flask, and charged with Pd(dppf)Cl₂ (150 mg, 0.21 mmol). The resultant mixture was purged with N₂ for 3 min and then heated at 90° C. for 5 h. After this time, the mixture was cooled to r.t., poured into water (50 mL), and extracted with ethyl acetate (50 mL×3). The combined organic extracts were washed with brine (60 mL), dried with anhydrous Na₂SO₄, filtered, and concentrated to dryness in vacuo to give a brown oil. The oil was then subjected to silica gel chromatography (0-100% EtOAc/pet ether) to give 3-(2-(6-chloropyridin-2-yl)thiazol-5-yl)-3-hydroxy-1-methylpyrrolidin-2-one as a yellow solid (180 mg, 20%). LCMS (ESI): Mass calcd. for C₁₃H₁₂ClN₃O₂S 309.0 m/z, found 309.8 [M+1]⁺.

### Intermediate 26. 3-(2-(6-Chloropyridin-2-yl)thiazol-4-yl)-3-hydroxy-1-methylpyrrolidin-2-one

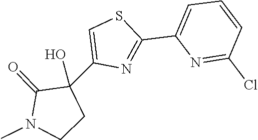 [SMILES: CN1CCC(O)(c2csc(-c3cccc(Cl)n3)n2)C1=O]

Step A. 6-Chloropyridine-2-carbothioamide. 6-Chloropicolinamide (1.0 g, 6.4 mmol), and THF (20 mL) were added to a nitrogen-purged 40 mL flask, and charged with Lawesson's reagent (1.3 g, 3.2 mmol). The resultant mixture was heated at 45° C. for 2 h. The mixture was concentrated to dryness under reduced pressure to give a yellow solid. The solid was then subjected to silica gel chromatography (0-35% EtOAc/pet ether) to give 6-chloropyridine-2-carbothioamide as a yellow solid (1 g, 90%). LCMS (ESI): Mass calcd. for C₆H₅ClN₂S 172.0 m/z, found 172.7 [M+1]⁺.

Step B. 3-(1-Ethoxyvinyl)-3-hydroxy-1-methylpyrrolidin-2-one. Ethoxyethene (12.0 mL, 125 mmol) and THF (50 mL) were added to an oven-dried and nitrogen-purged 250 mL three-necked round-bottomed flask, which was subsequently cooled to −72° C., and the resulting mixture treated with t-BuLi (40 mL, 1.3 M in pentane, 52 mmol), portion-wise over 5 min. The mixture was warmed to 0° C. and stirred until the color of the solution turned pale yellow. After this, 1-methylpyrrolidine-2,3-dione (2.00 g, 17.7 mmol) was added into the mixture. The mixture was stirred for 1 h at −72° C. and then for 12 h at room temperature. The reaction mixture was then diluted with EtOAc (150 mL), washed with H₂O (60 mL×3), dried with anhydrous Na₂SO₄, filtered, and concentrated to dryness in vacuo to give a yellow oil. The oil was then subjected to silica gel chromatography (0-80% EtOAc/pet ether) to give 3-(1-ethoxyvinyl)-3-hydroxy-1-methylpyrrolidin-2-one as a yellow oil (400 mg, 12%). LCMS (ESI): Mass calcd. for C₉H₁₅NO₃ 185.1 m/z, found 186.2 [M+1]⁺.

Step C. 3-(2-Bromoacetyl)-3-hydroxy-1-methylpyrrolidin-2-one. 3-(1-Ethoxyvinyl)-3-hydroxy-1-methylpyrrolidin-2-one (370 mg, 2.00 mmol) and acetone/H₂O=9:1 (5 mL) were added to a nitrogen-purged 40 mL flask, and charged with NBS (370 mg, 2.1 mmol) at 0° C. The resultant mixture was stirred at room temperature for 1 h. The reaction mixture was then diluted with water (20 mL), and then extracted with EtOAc (10 mL×3). The combined extracts were washed with brine (20 mL), dried over anhydrous Na₂SO₄, filtered, and concentrated to dryness in vacuo to give 3-(2-bromoacetyl)-3-hydroxy-1-methylpyrrolidin-2-one as a yellow oil (330 mg). LCMS (ESI): Mass calcd. for C₇H₁₀BrNO₃ 235.0 m/z, found 235.6 [M+1]⁺.

Step D. 3-(2-(6-Chloropyridin-2-yl)thiazol-4-yl)-3-hydroxy-1-methylpyrrolidin-2-one. 3-(2-Bromoacetyl)-3-hydroxy-1-methylpyrrolidin-2-one (330 mg, 1.4 mmol) and EtOH (7 mL) were added to a 40 mL flask, and charged with 6-chloropyridine-2-carbothioamide (270 mg, 1.6 mmol). The resultant mixture was stirred at 100° C. for 1.5 h. After this time, the mixture was cooled to r.t., diluted with EtOAc (50 mL), washed with H₂O (20 mL×3), dried over anhydrous Na₂SO₄, filtered, and concentrated to dryness in vacuo to give a yellow solid. The solid was then subjected to silica gel chromatography (0-100% EtOAc/pet ether) to give 3-(2-(6-chloropyridin-2-yl)thiazol-4-yl)-3-hydroxy-1-methylpyrrolidin-2-one as a yellow solid (270 mg, 57%). LCMS (ESI): Mass calcd. for C₁₃H₁₂ClN₃O₂S 309.0 m/z, found 309.9 [M+1]⁺.

### Intermediate 27. 3-(5-(6-chloropyridin-2-yl)thiazol-2-yl)-3-hydroxy-1-methylpyrrolidin-2-one

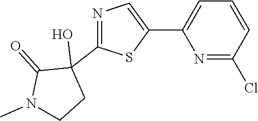 [SMILES: CN1CCC(O)(c2ncc(-c3cccc(Cl)n3)s2)C1=O]

Step A. 3-Hydroxy-1-methyl-3-(thiazol-2-yl)pyrrolidin-2-one. 2-Bromothiazole (1.0 mL, 11 mmol) and anhydrous THF (20 mL) were added to a 100 mL three-necked round-bottomed flask which was cooled to −78° C., then i-PrMgCl·LiCl (8.80 mL, 1.3 M in hexane, 11.4 mmol) was added dropwise. The resulting mixture was stirred at −78° C. for 30 min and then 1-methylpyrrolidine-2,3-dione (1.00 g, 8.8 mmol) in THF (10 mL) was added dropwise into the mixture. The resulting mixture was stirred for 3 h at room temperature. After this time, the mixture was quenched with water (40 mL) and extracted with ethyl acetate (50 mL×3). The combined organic extracts were washed with brine (50 mL), dried over anhydrous Na₂SO₄, filtered, and concentrated to dryness in vacuo to give a yellow oil. The oil was then subjected to silica gel chromatography (0-100% EtOAc/pet ether) to give 3-hydroxy-1-methyl-3-(thiazol-2-yl)pyrrolidin-2-one (460 mg, 25%) as a yellow solid. LCMS (ESI): Mass calcd for C₈H₁₀N₂O₂S 198.1 m/z, found 199.0 [M+H]⁺.

Step B. 3-(5-Bromothiazol-2-yl)-3-hydroxy-1-methylpyrrolidin-2-one. 3-Hydroxy-1-methyl-3-(thiazol-2-yl)pyrrolidin-2-one (500 mg, 2.52 mmol), NBS (500 mg, 2.8 mmol) and ACN (15 mL) were added to a 40 mL flask and the resulting mixture was heated for 16 h at 40° C. After this time, the mixture was concentrated to dryness in vacuo to give a white solid. The solid was then subjected to silica gel chromatography (0-100% EtOAc/pet ether) to give 3-(5-bromothiazol-2-yl)-3-hydroxy-1-methylpyrrolidin-2-one (530 mg, 69%) as a white solid. LCMS (ESI): Mass calcd. for C₈H₉BrN₂O₂S 276.0 m/z, found 276.9 [M+H]⁺. ¹H NMR (400 MHz, DMSO-d₆) δ 7.80 (s, 1H), 7.35-6.69 (m, 1H), 3.48-3.43 (m, 1H), 3.56-3.32 (m, 1H), 3.17 (s, 1H), 2.79 (s, 3H), 2.70-2.61 (m, 1H), 2.50 (br s, 1H), 2.29-2.17 (m, 1H).

Step C. 3-(5-(6-Chloropyridin-2-yl)thiazol-2-yl)-3-hydroxy-1-methylpyrrolidin-2-one. 3-(5-Bromothiazol-2-yl)-3-hydroxy-1-methylpyrrolidin-2-one (450 mg, 1.6 mmol), 2-chloro-6-(4,4,5,5-tetramethyl-1,3,2-dioxaborolan-2-yl)pyridine (790 mg, 3.3 mmol), K₂CO₃ (680 mg, 4.9 mmol), and 1,4-dioxane/H₂O (4:1) (20 mL) were added to a 100 mL round-bottomed flask, which was subsequently evacuated and refilled with argon (×3), then treated with Pd(dppf)Cl₂ (130 mg, 0.18 mmol) and the resulting mixture heated for 16 h at 110° C. After this time, the mixture was cooled to r.t., poured into water (30 mL), and extracted with ethyl acetate (30 mL×3). The combined organic extracts were washed with brine (40 mL), dried with anhydrous Na₂SO₄, filtered, and concentrated to dryness in vacuo to give a brown solid. The solid was then subjected to silica gel chromatography (0-10% MeOH/DCM) to give 3-(5-(6-chloropyridin-2-yl)thiazol-2-yl)-3-hydroxy-1-methylpyrrolidin-2-one (420 mg, 61%) as a black solid. LCMS (ESI): Mass calcd. for C₁₃H₁₂ClN₃O₂S 309.0 m/z, found 310.0 [M+H]⁺.

### Intermediate 28. N-(1-Methyl-1H-pyrazol-3-yl)-4-(6-(tributylstannyl)pyridin-2-yl)pyrimidin-2-amine

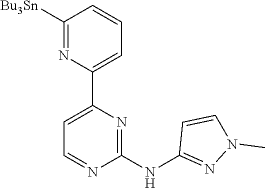

Step A. 4-(6-Bromopyridin-2-yl)-N-(1-methyl-1H-pyrazol-3-yl)pyrimidin-2-amine. N-(1-Methyl-1H-pyrazol-3-yl)-4-(tributylstannyl)pyrimidin-2-amine (Intermediate 24, 4.66 g, 10 mmol), 2,6-dibromopyridine (2.8 g, 12 mmol), TEA (2.8 mL, 20 mmol), and toluene (50 mL) were added to 250 mL round-bottomed flask, and charged with Pd(PPh₃)₄ (1.2 g, 1.0 mmol). The mixture was sparged with Ar for 5 minutes and then heated at 120° C. for 16 hours. The reaction vessel was removed from the oil bath and allowed to gradually cool to r.t. The reaction was then diluted with water (80 mL), extracted with ethyl acetate (100 mL×3), and the combined extracts washed with brine (60 mL), dried over anhydrous Na₂SO₄, filtered, and concentrated to dryness in vacuo to give a yellow solid. The yellow solid was then subjected to silica gel chromatography (0-100% ethyl acetate/petroleum ether) to give 4-(6-bromopyridin-2-yl)-N-(1-methyl-1H-pyrazol-3-yl)pyrimidin-2-amine (2.28 g, 43%) as a yellow solid. LCMS (ESI): Mass calcd. for C₁₃H₁₁BrN₆ 330.0 m/z, found 333.1 [M+H]⁺.

Step B. N-(1-Methyl-1H-pyrazol-3-yl)-4-(6-(tributylstannyl)pyridin-2-yl)pyrimidin-2-amine. 4-(6-Bromopyridin-2-yl)-N-(1-methyl-1H-pyrazol-3-yl)pyrimidin-2-amine (2.0 g, 6.0 mmol), LiCl (1.5 g, 35 mmol), and 1,4-dioxane (50 mL) were added to a 250 mL flask fitted with a reflux condenser and charged with 1, 1,1,2,2,2-hexabutyldistannane (5.83 g, 10.1 mmol). The resulting mixture was purged with N₂ three times and then treated with Pd₂(dba)₃ (273 mg, 0.298 mmol) and tricyclohexylphosphine (167 mg, 0.596 mmol). The resultant mixture was purged again with N₂ three times and then heated at 120° C. for 16 hours. After this time, the mixture was cooled to r.t. and quenched with saturated KF (20 mL), and stirred for 1 h. The reaction mixture was then diluted with water (30 mL), extracted with ethyl acetate (60 mL×3), and the combined extracts washed with brine (40 mL), dried over anhydrous Na₂SO₄, filtered, and concentrated to dryness in vacuo to give a yellow solid. The yellow solid was then subjected to silica gel chromatography (0-50% ethyl acetate/petroleum ether) to give N-(1-methyl-1H-pyrazol-3-yl)-4-(6-(tributylstannyl)pyridin-2-yl)pyrimidin-2-amine as a yellow solid (1.26 g, 37%). LCMS (ESI): Mass calcd. for C₂₅H₃₈N₆Sn 542.22 m/z, found 543.15 [M+H]⁺.

### Intermediate 29. 3-(3-Bromo-1-((2-(trimethylsilyl)ethoxy)methyl)-1H-pyrazol-5-yl)-3-hydroxy-1-methylpyrrolidin-2-one

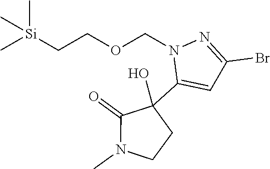 [SMILES: CN1CCC(O)(c2cc(Br)nn2COCC[Si](C)(C)C)C1=O]

Step A. 3,5-Dibromo-1-((2-(trimethylsilyl)ethoxy)methyl)-1H-pyrazole. 3,5-Dibromo-1H-pyrazole (8.0 g, 35 mmol), TEA (7.4 mL, 53 mmol), and DCM (100 mL) were added to a 250 mL flask. SEM-Cl (8.8 mL, 50 mmol) was added to the mixture. After stirring at rt for 30 minutes, the mixture was concentrated to dryness under reduced pressure to afford a colorless oil, which was then subjected to HPLC (Xtimate C18 column, 10 μm, 150×40 mm; 65-95% ACN/water with 0.04% NH₃·H₂O+10 mM NH₄HCO₃) to give, after lyophillization, 3,5-dibromo-1-((2-(trimethylsilyl)ethoxy)methyl)-1H-pyrazole (6.1 g, 48%) as a colorless oil. LCMS (ESI): Mass calcd. for C₉H₁₆Br₂N₂OSi 353.94 m/z, found 354.9 [M+H]⁺.

Step B. 3-(3-Bromo-1-((2-(trimethylsilyl)ethoxy)methyl)-1H-pyrazol-5-yl)-3-hydroxy-1-methylpyrrolidin-2-one. Tetrahydrofuran (30 mL) and 3,5-dibromo-1-((2-(trimethylsilyl)ethoxy)methyl)-1H-pyrazole (6.0 g, 17 mmol) were added to a N₂-purged 250 mL three-necked flask. The mixture was cooled down to −72° C. n-Butyllithium (7.41 mL, 2.5 M in hexane, 18.5 mmol) was added dropwise to the mixture. The mixture was stirred at −72° C. for 30 minutes, treated with a solution consisting of 1-methylpyrrolidine-2,3-dione (2.29 g, 20.2 mmol) and THF (50 mL), and then stirred at −72° C. for 1 hour. After this time, the mixture was quenched with H₂O (80 mL) and extracted with EtOAc (80 mL×3). The combined organic extracts were concentrated to dryness under reduced pressure to afford a brown oil, which was subjected to HPLC (Xtimate C₁₈ column, 10 μm, 150×40 mm; 40-70% (v/v) ACN/water (0.05% NH₃·H₂O+10 mM NH₄HCO₃)) to give, after lyophillization, 3-(3-bromo-1-((2-(trimethylsilyl)ethoxy)methyl)-1H-pyrazol-5-yl)-3-hydroxy-1-methylpyrrolidin-2-one as a white solid (3.2 g, 49%). LCMS (ESI): Mass calcd. for C₁₄H₂₄BrN₃O₃Si 389.1 m/z, found 390.0 [M+H]⁺.

### Intermediate 30. 3-(1-(6-Chloropyridin-2-yl)-1H-imidazol-4-yl)-3-hydroxy-1-methylpyrrolidin-2-one

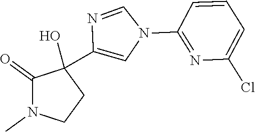 [SMILES: CN1CCC(O)(c2cn(-c3cccc(Cl)n3)cn2)C1=O]

Step A. 2-Chloro-6-(4-iodo-1H-imidazol-1-yl)pyridine. A mixture of 4-iodoimidazole (1.0 g, 5.2 mmol), 2,6-dichloropyridine (1.91 g, 12.9 mmol), cesium carbonate (5.04 g, 15.5 mmol) and DMA (20 mL) was heated at 100° C. for 2 hr. The reaction mixture was cooled to rt and then poured into 150 mL of water resulting in the formation of a precipitate. The precipitate was collected by vacuum filtration and washed with water. The filter cake was collected and purified by FCC (silica, 40-100% EtOAc in hexanes) to afford 2-chloro-6-(4-iodo-1H-imidazol-1-yl)pyridine (685 mg,43%) as a white solid. ¹H NMR (500 MHz, DMSO-d₆) δ 8.51-8.40 (m, 1H), 8.18-8.12 (m, 1H), 8.08 (t, J=7.9 Hz, 1H), 7.82 (d, J=8.0 Hz, 1H), 7.52 (d, J=7.8 Hz, 1H). LCMS (ESI+): m/z=306.0.

Step B. 3-(1-(6-Chloropyridin-2-yl)-1H-imidazol-4-yl)-3-hydroxy-1-methylpyrrolidin-2-one. To a solution of 2-chloro-6-(4-iodo-1H-imidazol-1-yl)pyridine (540 mg, 1.768 mmol) in THF (10 mL) at 0° C. was added isopropylmagnesium chloride-lithium chloride complex (1.36 mL, 1.3 M in THF, 1.77 mmol) dropwise over 2 minutes. The resulting mixture was stirred for 10 minutes and then 1-methylpyrrolidine-2,3-dione (220 mg, 1.94 mmol) was added as a solid in one portion. The resulting mixture was stirred for 50 minutes and then diluted with saturated aqueous NH₄Cl solution, water, and extracted with ethyl acetate. The organic extract was dried with MgSO₄, filtered, and concentrated. The residue was purified by FCC (silica, 0-10% MeOH in EtOAc) to afford 3-(1-(6-chloropyridin-2-yl)-1H-imidazol-4-yl)-3-hydroxy-1-methylpyrrolidin-2-one (81 mg, 15.6%) as a white solid. ¹H NMR (400 MHz, CDCl₃) δ 8.25 (d, J=1.5 Hz, 1H), 7.73 (t, J=7.9 Hz, 1H), 7.69 (d, J=1.5 Hz, 1H), 7.22 (d, J=7.8 Hz, 1H), 7.19 (d, J=8.0 Hz, 1H), 4.61 (s, 1H), 3.64-3.52 (m, 1H), 3.51-3.39 (m, 1H), 2.96 (s, 3H), 2.84-2.71 (m, 1H), 2.48-2.29 (m, 1H). LCMS (ESI+): m/z=293.1.

### Intermediate 31. (R)-3-(3-(6-(2-Chloropyrimidin-4-yl)pyridin-2-yl)isoxazol-5-yl)-3-hydroxy-1-methylpyrrolidin-2-one

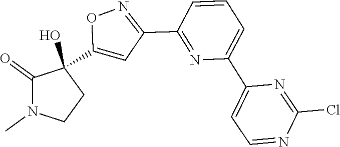 [SMILES: CN1CC[C@@](O)(c2cc(-c3cccc(-c4ccnc(Cl)n4)n3)no2)C1=O]

A solution of TMPMgCl·LiCl (1.00 M in THF, 813 mL, 813 mmol) was cooled in a dry-ice EtOH bath. A solution of 2-chloropyrimidine (84.6 g, 739 mmol) in THF (1000 mL) was added dropwise over 2 h by constant pressure funnel. The resulting solution was stirred in a dry-ice EtOH bath for a further 2 hr. ZnCl₂ (1.00 M in THF, 1.11 L, 1.11 mol) was added with a constant pressure funnel over 2 h. Then the mixture was slowly warmed to 25° C. and stirred for 12 h. The resulting solution was added by constant pressure funnel over 6 h to another flask containing (R)-3-(3-(6-bromopyridin-2-yl)isoxazol-5-yl)-3-hydroxy-1-methylpyrrolidin-2-one (from Step A in the preparation of Intermediate 13, 100 g, 295.72 mmol), Pd(PPh₃)₄ (42.7 g, 36.9 mmol) and THF (800 mL) at 60° C. The resulting mixture was stirred for an additional hour. The reaction was cooled to 25° C., poured into saturated aqueous NH₄Cl (4000 mL) and extracted with ethyl acetate (2000 mL×3). The combined organic extracts were dried with anhydrous Na₂SO₄, filtered, and concentrated. The residue was triturated with MeOH (500 mL) at 25° C. for 12 h. The reaction was carried out by eight batches. (R)-3-(3-(6-(2-Chloropyrimidin-4-yl)pyridin-2-yl)isoxazol-5-yl)-3-hydroxy-1-methylpyrrolidin-2-one (670 g, 1.78 mol, 61.0%) was obtained as a yellow solid. ¹H NMR (400 MHz, DMSO-d₆) δ 8.97 (d, J=5.1 Hz, 1H), 8.55 (d, J=5.1 Hz, 1H), 8.50-8.43 (m, 1H), 8.24-8.17 (m, 2H), 7.22 (s, 1H), 6.78 (s, 1H), 3.57-3.40 (m, 2H), 2.85 (s, 3H), 2.62 (ddd, J=5.1, 7.8, 13.2 Hz, 1H), 2.30 (ddd, J=5.8, 7.8, 13.4 Hz, 1H), 1.98 (s, 1H). LCMS (ESI⁺): m/z=371.9.

### Intermediate 32. (R)-3-(1-(6-(2-Chloropyrimidin-4-yl)pyridin-2-yl)-1H-1,2,3-triazol-4-yl)-3-hydroxy-1-methylpyrrolidin-2-one

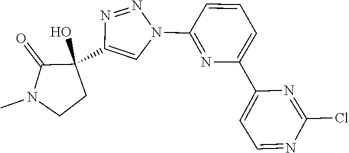 [SMILES: CN1CC[C@@](O)(c2cn(-c3cccc(-c4ccnc(Cl)n4)n3)nn2)C1=O]

(R)-3-(1-(6-(2-Chloropyrimidin-4-yl)pyridin-2-yl)-1H-1,2,3-triazol-4-yl)-3-hydroxy-1-methylpyrrolidin-2-one. To a stirred solution of 2-chloropyrimidine (103 g, 899 mmol) in tetrahydrofuran (1200 mL) was added TMPMgCl·LiCl (1.0 M in THF, 1174 mL) dropwise at −72° C. under a nitrogen atmosphere. The resulting mixture was stirred for 2 h at −72° C. under a nitrogen atmosphere. To the above mixture was added zinc chloride (1927 mL, 1349 mmol, 0.7 M in THF) dropwise at −72° C. The resulting mixture was stirred overnight at room temperature. A solution of (R)-3-(1-(6-bromopyridin-2-yl)-1H-1,2,3-triazol-4-yl)-3-hydroxy-1-methylpyrrolidin-2-one (152 g, 450 mmol, Intermediate 51) and tetrakis(triphenylphosphine)palladium(0) (52 g, 45 mmol) in tetrahydrofuran (1500 mL) was added to the above mixture dropwise at 60° C. under a nitrogen atmosphere. The final reaction mixture was stirred for 1 h at 60° C. under a nitrogen atmosphere. The mixture was allowed to cool down to room temperature. The reaction was quenched by the addition of water (3 L) at room temperature. The resulting mixture was extracted with EtOAc (3×2 L). The combined organic layers were washed with brine (1×1 L) and dried over anhydrous Na₂SO₄. After filtration, the filtrate was concentrated under reduced pressure. The residue was purified by silica gel column chromatography, elution with CH₂Cl₂/MeOH (20:1), to afford (3R)-3-{1-[6-(2-chloropyrimidin-4-yl)pyridin-2-yl]-1,2,3-triazol-4-yl}-3-hydroxy-1-methylpyrrolidin-2-one (126.1 g, 75%) as an off-white solid. H NMR: (300 MHz, DMSO-d₆) δ 9.11-8.98 (m, 2H), 8.64 (t, J=5.1 Hz, 1H), 8.49 (dd, J=5.8, 2.8 Hz, 1H), 8.40-8.31 (m, 2H), 6.33 (s, 1H), 3.50 (t, J=6.6 Hz, 2H), 2.91-2.69 (m, 4H), 2.29 (dt, J=13.4, 6.9 Hz, 1H). LCMS (ESI⁺): m/z=372.

### Intermediate 33. (1R,4R,5S)-4-(3-(6-Bromopyridin-2-yl)isoxazol-5-yl)-4-hydroxy-2-methyl-2-azabicyclo[3.1.0]hexan-3-one

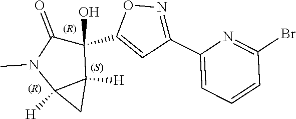

A solution of (Z)-6-bromo-N-hydroxypicolinimidoyl chloride (Intermediate 4, 1 g, 4.2 mmol), (1R,4R,5S)-4-ethynyl-4-hydroxy-2-methyl-2-azabicyclo[3.1.0]hexan-3-one (0.8 g, 5.1 mmol), NaHCO₃ (1 g, 12.7 mmol), EtOAc (4.2 mL, 0.2 M, 0.83 mmol), and water (distilled, 0.83 mL, 1.7 mmol) was stirred at room temperature for 1.5 hours. The reaction mixture was diluted with 70 mL water and extracted with ethyl acetate (3×70 mL). The organic extracts were dried over MgSO₄, filtered, and concentrated under reduced pressure. The product was purified via FCC (12 g silica, 0-15% EtOAc in hexanes) to yield (1R,4R,5S)-4-(3-(6-bromopyridin-2-yl)isoxazol-5-yl)-4-hydroxy-2-methyl-2-azabicyclo[3.1.0]hexan-3-one (846.9 mg, 57%). ¹H NMR (500 MHz, CDCl₃) δ 8.04-8.00 (m, 1H), 7.65 (t, J=7.8 Hz, 1H), 7.54 (dd, J=7.9, 0.9 Hz, 1H), 7.06-7.04 (m, 1H), 3.32-3.28 (m, 1H), 3.17-3.13 (m, 1H), 2.98 (s, 3H), 2.17-2.11 (m, 1H), 1.10-1.05 (m, 1H). MS (ESI⁺): m/z=351.9.

### Intermediate 34. (1R,4R,5S)-4-(3-(6-(2-Chloropyrimidin-4-yl)pyridin-2-yl)isoxazol-5-yl)-4-hydroxy-2-methyl-2-azabicyclo[3.1.0]hexan-3-one

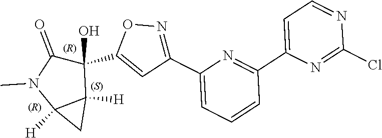

A solution of (1R,4R,5S)-4-(3-(6-bromopyridin-2-yl)isoxazol-5-yl)-4-hydroxy-2-methyl-2-azabicyclo[3.1.0]hexan-3-one (Intermediate 33, 75 mg, 0.2 mmol), 2-chloro-4-(tributylstannyl)pyrimidine (100 mg, 0.2 mmol), Pd(PPh₃)₄ (12 mg, 0.01 mmol), DMF (1 mL, 14.5 mmol), and lithium chloride (11 mg, 0.26 mmol) was subjected to microwave irradiation under a nitrogen atmosphere at 130° C. for 1 hour. The reaction mixture was allowed to cool to room temperature. The reaction mixture was diluted in EtOAc and water and stirred vigorously with potassium fluoride (17 mg, 0.26 mmol) for 1 hour. The solids were filtered off before transferring the mixture to a separatory funnel for aqueous extraction with EtOAc (3×75 mL) and water (100 mL). The organic layer was dried over MgSO₄, filtered, and concentrated under reduced pressure. The product was purified via FCC (4 g silica, 0-100% ethyl acetate in hexanes) to yield (1R,4R,5S)-4-(3-(6-(2-chloropyrimidin-4-yl)pyridin-2-yl)isoxazol-5-yl)-4-hydroxy-2-methyl-2-azabicyclo[3.1.0]hexan-3-one (35 mg, 43%). ¹H NMR (500 MHz, DMSO-d₆) δ 8.98 (d, J=5.1 Hz, 1H), 8.58 (d, J=5.1 Hz, 1H), 8.50 (dd, J=6.7, 2.1 Hz, 1H), 8.24-8.23 (m, 1H), 8.22-8.20 (m, 1H), 7.24 (s, 1H), 6.68 (s, 1H), 3.46-3.43 (m, 1H), 2.86 (s, 3H), 2.11-2.06 (m, 1H), 0.98-0.94 (m, 1H), 0.85-0.81 (m, 1H). MS (ESI⁺): m/z=383.2.

### Intermediate 35. (3R,5R)-3-(3-(6-Bromopyridin-2-yl)isoxazol-5-yl)-3-hydroxy-1,5-dimethylpyrrolidin-2-one

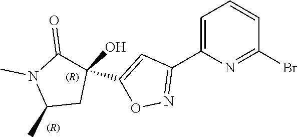

A solution of (Z)-6-bromo-N-hydroxypicolinimidoyl chloride (Intermediate 4, 196 mg, 0.832 mmol), (3R,5R)-3-ethynyl-3-hydroxy-1,5-dimethylpyrrolidin-2-one (153 mg, 1 mmol), NaHCO₃ (210 mg, 2.5 mmol), EtOAc (1 mL, 0.2 M, 0.2 mmol), and water (distilled, 1 mL, 2 mmol) was stirred at room temperature for 1.5 hours. The reaction mixture was diluted in 70 mL water and extracted with ethyl acetate (3×70 mL). The organic extracts were dried over MgSO₄, filtered, and concentrated under reduced pressure. The product was purified via FCC (12 g silica, 0-15% EtOAc in hexanes) to yield (3R,5R)-3-(3-(6-bromopyridin-2-yl)isoxazol-5-yl)-3-hydroxy-1,5-dimethylpyrrolidin-2-one (163 mg, 56%). ¹H NMR (500 MHz, DMSO-d₆) δ 8.04 (dd, J=7.6, 0.9 Hz, 1H), 7.92 (t, J=7.8 Hz, 1H), 7.80 (dd, J=7.9, 0.8 Hz, 1H), 6.92 (s, 1H), 6.75 (s, 1H), 3.67 (h, J=6.4 Hz, 1H), 2.83-2.80 (m, 1H), 2.79 (s, 3H), 1.88 (dd, J=13.4, 6.4 Hz, 1H), 1.29 (d, J=6.3 Hz, 3H). MS (ESI⁺): m/z=351.9.

### Intermediate 36. (3R,5R)-3-(3-(6-(2-Chloropyrimidin-4-yl)pyridin-2-yl)isoxazol-5-yl)-3-hydroxy-1,5-dimethylpyrrolidin-2-one

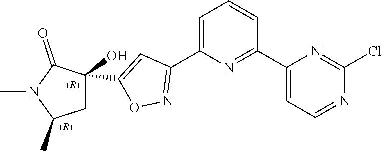

A solution of (3R,5R)-3-(3-(6-bromopyridin-2-yl)isoxazol-5-yl)-3-hydroxy-1,5-dimethylpyrrolidin-2-one (Intermediate 35, 415 mg, 1.18 mmol), 2-chloro-4-(tributylstannyl)pyrimidine (580 mg, 1.4 mmol), Pd(PPh₃)₄ (68 mg, 0.059 mmol), DMF (5 mL, 65 mmol), and lithium chloride (60 mg, 1.4 mmol) was subjected to microwave irradiation under a nitrogen atmosphere at 130° C. for 1 hour. The mixture was allowed to cool to room temperature. The reaction mixture was diluted in EtOAc and water and stirred vigorously with potassium fluoride (16 mg, 0.3 mmol) for 1 hour. The solids were filtered off before transferring the mixture to a separatory funnel for aqueous extraction with (3×75 mL) EtOAc and water (100 mL). The organic extracts were dried over MgSO₄, filtered, and concentrated under reduced pressure. The product was purified via FCC (4 g silica, 0-100% ethyl acetate in hexanes) to yield (3R,5R)-3-(3-(6-(2-chloropyrimidin-4-yl)pyridin-2-yl)isoxazol-5-yl)-3-hydroxy-1,5-dimethylpyrrolidin-2-one (213 mg, 47%). 1H NMR (500 MHz, DMSO-d₆) δ 8.99 (d, J=5.1 Hz, 1H), 8.57 (d, J=5.1 Hz, 1H), 8.49 (dd, J=5.5, 3.4 Hz, 1H), 8.23 (s, 1H), 8.22 (d, J=2.2 Hz, 1H), 7.21 (s, 1H), 6.77 (s, 1H), 3.70 (q, J=6.4 Hz, 1H), 2.84 (dd, J=13.4, 6.9 Hz, 1H), 2.81 (s, 3H), 1.92 (dd, J=13.4, 6.4 Hz, 1H), 1.31 (d, J=6.3 Hz, 3H). MS (ESI⁺): m/z=385.3.

### Intermediate 37. (R)-3-(3-(6-Chloropyrazin-2-yl)isoxazol-5-yl)-4,4-difluoro-3-hydroxy-1-methylpyrrolidin-2-one

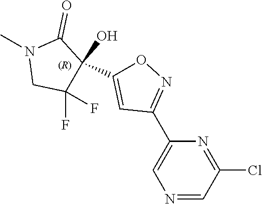

A solution of (Z)-6-chloro-N-hydroxypyrazine-2-carbimidoyl chloride (Intermediate 39, 100 mg, 0.5 mmol), (3R)-3-ethynyl-4,4-difluoro-3-hydroxy-1-methyl-pyrrolidin-2-one (145 mg, 0.828 mmol), NaHCO₃ (131 mg, 1.56 mmol), EtOAc (2.6 mL, 0.52 mmol), and water, distilled (0.26 mL, 0.52 mmol) was stirred at room temperature for 18 hours. The reaction mixture was diluted in 40 mL water and extracted with ethyl acetate (3×40 mL). The organic extracts were dried over MgSO₄, filtered, and concentrated under reduced pressure. The product was purified via FCC (4 g silica, 0-15% MeOH in DCM) to yield (R)-3-(3-(6-chloropyrazin-2-yl)isoxazol-5-yl)-4,4-difluoro-3-hydroxy-1-methylpyrrolidin-2-one (182 mg). ¹H NMR (500 MHz, DMSO-d₆) δ 9.26 (d, J=0.6 Hz, 1H), 8.97 (d, J=0.6 Hz, 1H), 8.01 (s, 1H), 7.22 (s, 1H), 4.02 (dd, J=12.9, 11.7 Hz, 3H), 3.82-3.77 (m, 5H), 2.96 (s, 3H). MS (ESI⁺): m/z=331.0.

### Intermediate 38. (R)-3-(3-(6-Chloropyrazin-2-yl)isoxazol-5-yl)-3-hydroxy-1-methylpyrrolidin-2-one

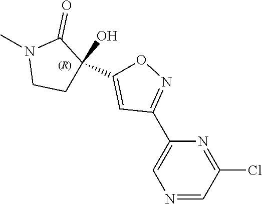

A solution of (Z)-6-chloro-N-hydroxypyrazine-2-carbimidoyl chloride (Intermediate 39, 100 mg, 0.5 mmol), (3R)-3-ethynyl-3-hydroxy-1-methyl-pyrrolidin-2-one (145 mg, 1 mmol), NaHCO₃ (131 mg, 1.5 mmol), EtOAc (2.6 mL, 0.5 mmol), and water (distilled, 0.26 mL, 0.5 mmol) was stirred at room temperature for 18 hours. The reaction mixture was diluted in 40 mL water and extracted with ethyl acetate (3×40 mL). The organic extracts were dried over MgSO₄, filtered, and concentrated under reduced pressure. The product was purified via FCC (4 g silica, 0-15% MeOH in DCM).. The isolated material was a 1:1 mixture of the alkyne starting material and (R)-3-(3-(6-chloropyrazin-2-yl)isoxazol-5-yl)-3-hydroxy-1-methylpyrrolidin-2-one (81 mg, 53%) and was used without further purification. ¹H NMR (500 MHz, DMSO-d₆) δ 9.23 (d, J=0.6 Hz, 1H), 7.07 (s, 1H), 6.81 (s, 1H), 6.30 (s, 1H), 3.52-3.40 (m, 3H), 3.30-3.22 (m, 2H), 2.84 (s, 3H). MS (ESI⁺): m/z=295.0.

### Intermediate 39. (Z)-6-Chloro-N-hydroxypyrazine-2-carbimidoyl chloride

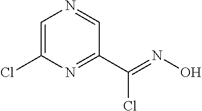 [SMILES: O/N=C(\Cl)c1cncc(Cl)n1]

Step A. (E)-6-Chloropyrazine-2-carbaldehyde oxime. A solution of 6-chloropyrazine-2-carbaldehyde (1000 mg, 7 mmol), hydroxylamine hydrochloride (536 mg, 7.7 mmol), EtOH (23 mL, 7 mmol), and sodium acetate (1150 mg, 14 mmol) was stirred under a nitrogen atmosphere at room temperature for 3 hours. The reaction mixture was diluted with 75 mL water and washed with ethyl acetate (3×75 mL). The organic extracts were dried over MgSO₄, filtered, and concentrated under reduced pressure to yield the title compound as a brown solid (1070 mg, 97%). ¹H NMR (500 MHz, DMSO-d₆) δ 12.27 (s, 1H), 8.96 (s, 1H), 8.77 (d, J=0.6 Hz, 1H), 8.14 (s, 1H). MS (ESI⁺): m/z=158.0.

Step B. (Z)-6-Chloro-N-hydroxypyrazine-2-carbimidoyl chloride. A solution of (E)-6-chloropyrazine-2-carbaldehyde oxime (1 g, 7 mmol), N-chlorosuccinimide (1060 mg, 8 mmol), and DMF (16 mL, 206 mmol) was stirred at rt for 1 hour. The reaction mixture changed color from brown to a bright orange. The reaction mixture was diluted with water (50 mL) and washed with ethyl acetate (3×50 mL). The organic extracts were dried over MgSO₄, filtered and concentrated under reduced pressure to yield an orange solid, (Z)-6-chloro-N-hydroxypyrazine-2-carbimidoyl chloride (950 mg, 75%). ¹H NMR (500 MHz, DMSO-d₆) δ 13.25 (s, 1H), 9.08 (d, J=0.5 Hz, 1H), 8.89 (d, J=0.6 Hz, 1H). MS (ESI⁺): m/z=193.9.

### Intermediate 40. (1R,4R,5S)-4-Hydroxy-2-methyl-4-(3-(6-(2-(methylsulfonyl)pyrimidin-4-yl)pyridin-2-yl)isoxazol-5-yl)-2-azabicyclo[3.1.0]hexan-3-one

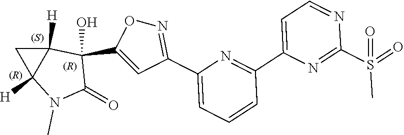

Step A. (1R,4R,5S)-4-Hydroxy-2-methyl-4-(3-(6-(2-(methylthio)pyrimidin-4-yl)pyridin-2-yl)isoxazol-5-yl)-2-azabicyclo[3.1.0]hexan-3-one. A mixture of (1R,4R,5S)-4-(3-(6-bromopyridin-2-yl)isoxazol-5-yl)-4-hydroxy-2-methyl-2-azabicyclo[3.1.0]hexan-3-one (Intermediate 33, 500 mg, 1.4 mmol), 2-(methylthio)-4-(tributylstannyl)pyrimidine (771 mg, 1.9 mmol), Pd(PPh₃)₄ (83 mg, 0.07 mmol), DMF (5 mL, 65 mmol), and lithium chloride (73 mg, 1.7 mmol) was subjected to microwave irradiation under a nitrogen atmosphere at 130° C. for 1.5 hours. The reaction mixture was diluted in EtOAc and water and stirred vigorously with potassium fluoride (166 mg, 2.86 mmol) for 1 hour. The solids were filtered off before transferring the mixture to a separatory funnel for aqueous extraction with (3×75 mL) EtOAc and water (100 mL). The organic extracts were dried over MgSO₄, filtered, and concentrated under reduced pressure. The product was then purified via FCC (12 g silica, 0-100% EtOAc in hexanes) to yield (1R,4R,5S)-4-hydroxy-2-methyl-4-(3-(6-(2-(methylthio)pyrimidin-4-yl)pyridin-2-yl)isoxazol-5-yl)-2-azabicyclo[3.1.0]hexan-3-one (187 mg, 33%). ¹H NMR (500 MHz, DMSO-d₆) δ 8.84 (d, J=5.1 Hz, 1H), 8.57-8.53 (m, 1H), 8.23 (d, J=5.1 Hz, 1H), 8.21-8.19 (m, 2H), 7.63-7.61 (m, 1H), 7.22 (s, 1H), 6.67 (s, 1H), 3.46-3.43 (m, 1H), 2.85 (s, 3H), 2.64 (s, 3H), 2.11-2.06 (m, 1H), 0.99-0.94 (m, 1H), 0.84-0.81 (m, 1H). MS (ESI⁺): m/z=396.0.

Step B. (1R,4R,5S)-4-Hydroxy-2-methyl-4-(3-(6-(2-(methylsulfonyl)pyrimidin-4-yl)pyridin-2-yl)isoxazol-5-yl)-2-azabicyclo[3.1.0]hexan-3-one. A mixture of (1R,4R,5S)-4-hydroxy-2-methyl-4-(3-(6-(2-(methylthio)pyrimidin-4-yl)pyridin-2-yl)isoxazol-5-yl)-2-azabicyclo[3.1.0]hexan-3-one (187 mg, 0.47 mmol), potassium peroxymonosulfate (872 mg, 1.42 mmol), acetone (1 mL, 13 mmol), water (distilled, 1 mL, 55 mmol), and MeOH (1 mL, 24 mmol) was stirred at room temperature overnight. The reaction mixture was diluted with 50 mL of water and stirred for 10 minutes. The precipitate was collected by vacuum filtration and dried to yield (1R,4R,5S)-4-hydroxy-2-methyl-4-(3-(6-(2-(methylsulfonyl)pyrimidin-4-yl)pyridin-2-yl)isoxazol-5-yl)-2-azabicyclo[3.1.0]hexan-3-one (112 mg, 55%) as a white solid. ¹H NMR (500 MHz, DMSO-d₆) δ 9.28 (d, J=5.2 Hz, 1H), 8.84 (d, J=5.2 Hz, 1H), 8.66 (dd, J=5.0, 3.9 Hz, 1H), 8.28 (s, 1H), 8.27 (d, J=1.4 Hz, 1H), 7.29 (s, 1H), 6.69 (s, 1H), 3.56 (s, 3H), 3.48-3.44 (m, 1H), 2.87 (s, 3H), 2.13-2.07 (m, 1H), 1.00-0.95 (m, 1H), 0.86-0.83 (m, 1H). MS (ESI⁺): m/z=428.0.

### Intermediate 41. (R)-3-(1-(6-(2-Chloropyrimidin-4-yl-6-d)pyridin-2-yl)-1H-1,2,3-triazol-4-yl)-3-hydroxy-1-methylpyrrolidin-2-one

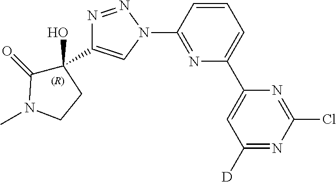

A mixture of 2-chloropyrimidine-4,6-d₂ (250 mg, 2.15 mmol, Intermediate 49) and THF (2 mL) was cooled to −72° C. and then TMPMgCl·LiCl (2.5 mL, 2.5 mmol, 1 M in THF) was added. After 2 h, the resulting mixture was treated with ZnCl₂ (5.0 mL, 3.5 mmol, 0.7 M in THF). Separately, (R)-3-(1-(6-bromopyridin-2-yl)-1H-1,2,3-triazol-4-yl)-3-hydroxy-1-methylpyrrolidin-2-one (Intermediate 51, 210 mg, 0.621 mmol), Pd(PPh₃)₄ (60 mg, 0.052 mmol), THF (5 mL) were heated for 1 h at 60° C. The two solutions were then combined and heated at 60° C. for 1 h, cooled to rt, diluted with water (30 mL) and extracted with EtOAc (20 mL×3). The combined organic extracts were washed with brine (50 mL), dried over anhydrous Na₂SO₄, filtered, and concentrated to dryness in vacuo to give a yellow solid. The yellow solid was then subjected to FCC (0-100% EA/petroleum ether) to give (R)-3-(1-(6-(2-chloropyrimidin-4-yl-6-d)pyridin-2-yl)-1H-1,2,3-triazol-4-yl)-3-hydroxy-1-methylpyrrolidin-2-one (80 mg, 8.1%) as a white solid. LCMS (ESI): mass calcd. for C₁₆H₁₃ClDN₇O₂ 372.10 m/z, found 373.2 [M+1]⁺.

### Intermediate 42. 4′H,6′H-Spiro[cyclopropane-1,5′-pyrrolo[1,2-b]pyrazol]-2′-amine

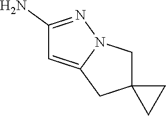 [SMILES: Nc1cc2n(n1)CC1(CC1)C2]

Step A: tert-Butyl((1-(iodomethyl)cyclopropyl)methoxy)dimethylsilane. To a solution of (1-(((tert-butyldimethylsilyl)oxy)methyl)cyclopropyl)methanol (210 g, 970 mmol) in THF (1200 mL) at 25° C. was added imidazole (99.1 g, 1.46 mol) and PPh₃ (382 g, 1.46 mol). Then I₂ (320 g, 1.26 mol, 254 mL) in THF (300 mL) was added dropwise at 0° C. The mixture was stirred at 25° C. for 12 h and then diluted with H₂O and extracted with ethyl acetate. The combined organic extracts were washed with brine, dried over anhydrous Na₂SO₄, filtered and concentrated under reduced pressure. The residue obtained was purified by FCC to afford tert-butyl((1-(iodomethyl)cyclopropyl)methoxy)dimethylsilane (161 g, 51%) as a yellow liquid. ¹H NMR (400 MHz, CDCl₃) δ 3.64-3.60 (m, 2H), 3.59-3.53 (m, 2H), 2.58 (br s, 1H), 0.90 (s, 9H), 0.56-0.49 (m, 2H), 0.47-0.41 (m, 2H), 0.07 (s, 6H).

Step B: 5-((1-(((tert-Butyldimethylsilyl)oxy)methyl)cyclopropyl)methyl)-1-(tetrahydro-2H-pyran-2-yl)-1H-pyrazole. To a solution of 1-(tetrahydro-2H-pyran-2-yl)-1H-pyrazole (65.0 g, 427 mmol) in THF (650 mL) at −65° C. was added n-BuLi (2.5 M in hexanes, 205 mL) dropwise and the resulting mixture was stirred for 1 hr. Then a solution of tert-butyl((1-(iodomethyl)cyclopropyl)methoxy)dimethylsilane (139 g, 427 mmol) in THF (150 mL) was added dropwise. The solution was warmed to 25° C. and stirred for 12 h. The reaction mixture was diluted with aq. saturated NH₄Cl solution and then extracted with ethyl acetate. The combined organic extracts were washed with brine, dried over anhydrous Na₂SO₄, filtered and concentrated under reduced pressure. The residue obtained was purified by FCC (0-4% EtOAc/petroleum ether) to afford 5-((1-(((tert-butyldimethylsilyl)oxy)methyl)cyclopropyl)methyl)-1-(tetrahydro-2H-pyran-2-yl)-1H-pyrazole (54.0 g, 36.1%) as a yellow oil. ¹H NMR (400 MHz, DMSO-d₆) δ 7.43-7.24 (m, 1H), 6.12 (d, J=2.0 Hz, 1H), 5.40-5.23 (m, 1H), 3.91-3.83 (m, 1H), 3.63-3.56 (m, 1H), 3.43-3.36 (m, 2H), 2.89 (dd, J=15.6, 3.2 Hz, 1H), 2.65 (dd, J=15.6, 3.2 Hz, 1H), 2.40-2.24 (m, 1H), 2.05-1.93 (m, 1H), 1.85-1.74 (m, 1H), 1.70-1.58 (m, 1H), 1.55-1.45 (m, 2H), 0.86 (d, J=3.6 Hz, 9H), 0.48-0.32 (m, 4H), −0.02 (t, J=3.2 Hz, 6H).

Step C: (1-((1H-Pyrazol-5-yl)methyl)cyclopropyl)methanol. A solution of 5-((1-(((tert-butyldimethylsilyl)oxy)methyl)cyclopropyl)methyl)-1-(tetrahydro-2H-pyran-2-yl)-1H-pyrazole (50.0 g, 143 mmol) and HCl (4 M in water, 600 mL) was stirred at 25° C. for 12 h. The reaction mixture was poured into ice water and then the pH was adjusted to 8-9 with saturated aqueous Na₂CO₃. The mixture was extracted with ethyl acetate and the combined organic extracts were washed with brine, dried over anhydrous Na₂SO₄, filtered and concentrated under reduced pressure. The residue obtained was purified by FCC (0-65% EtOAc/petroleum ether) to afford (1-((1H-pyrazol-5-yl)methyl)cyclopropyl)methanol (17.0 g, 78.3%) as a yellow solid. LCMS (ESI⁺): m/z (M+H⁺)=153.1. ¹H NMR: (400 MHz, DMSO-d₆) δ 12.39 (br s, 1H), 7.68-7.17 (m, 1H), 6.02 (s, 1H), 4.56-4.39 (m, 1H), 3.25-3.10 (m, 2H), 2.64 (s, 2H), 0.34 (br s, 4H).

Step D: 4′H,6′H-Spiro[cyclopropane-1,5′-pyrrolo[1,2-b]pyrazole]. To a solution of (1-((1H-pyrazol-5-yl)methyl)cyclopropyl)methanol (5.00 g, 32.9 mmol) in DCM (35.0 mL) was added 2-(tributyl-λ5-phosphaneylidene)acetonitrile (17.4 g, 72.3 mmol). The mixture was heated at 50° C. for 3 h. The reaction mixture was then concentrated under reduced pressure. The residue obtained was purified by FCC (0-10% EtOAc/petroleum ether) to afford 4′H,6′H-spiro[cyclopropane-1,5′-pyrrolo[1,2-b]pyrazole](3.00 g, 21%) as a yellow liquid. LCMS (ESI⁺): m/z (M+H⁺)=135.1. ¹H NMR: (400 MHz, DMSO-d₆) δ 7.43 (d, J=2.00 Hz, 1H), 6.01-5.93 (m, 1H), 3.99 (s, 2H), 2.83 (s, 2H), 0.83-0.77 (m, 2H), 0.77-0.71 (m, 2H).

Step E: 2′-Nitro-4′H,6′H-spiro[cyclopropane-1,5′-pyrrolo[1,2-b]pyrazole]. To a solution of AcOH (150 mL) cooled to 10-15° C. was added Ac₂O (150 mL) dropwise. Then a solution of 4′H,6′H-spiro[cyclopropane-1,5′-pyrrolo[1,2-b]pyrazole](3.00 g, 22.4 mmol) in AcOH (20.0 mL) was added dropwise at 5-10° C. HNO₃ (23.2 g, 257 mmol, 16.5 mL, 70% purity) was then added dropwise. The mixture was stirred at 25° C. for 2 h. The reaction mixture was diluted with ice-water and the pH was adjusted to 7-8 with Na₂CO₃ (aq.). The mixture was extracted with ethyl acetate and the combined organic extracts were washed with brine, dried over anhydrous Na₂SO₄, filtered and concentrated under reduced pressure. The residue obtained was triturated with n-hexane (30.0 mL) at 25° C. for 12 h to afford 2′-nitro-4′H,6′H-spiro[cyclopropane-1,5′-pyrrolo[1,2-b]pyrazole](3.70 g, 92%). LCMS (ESI⁺): m/z (M+H⁺)=180.1. ¹H NMR: (400 MHz, DMSO-d₆) δ 6.87 (s, 1H), 4.18 (s, 2H), 2.95 (s, 2H), 0.89-0.83 (m, 2H), 0.82-0.77 (m, 2H).

Step F: 4′H,6′H-Spiro[cyclopropane-1,5′-pyrrolo[1,2-b]pyrazol]-2′-amine. To a solution of 2′-nitro-4′H,6′H-spiro[cyclopropane-1,5′-pyrrolo[1,2-b]pyrazole](3.60 g, 20.1 mmol) in MeOH (5.00 mL) was added Pd/C (400 mg, 10 wt % Pd) under an Ar atmosphere. The suspension was degassed and purged with H² 3 times. The mixture was stirred under a H₂ atmosphere (50 psi) at 25° C. for 4 h. The reaction mixture was then filtered and the filtrate was concentrated. The residue obtained was purified by FCC (0-90% EtOAc/petroleum ether) to afford 4′H,6′H-spiro[cyclopropane-1,5′-pyrrolo[1,2-b]pyrazol]-2′-amine (2.00 g, 66%) as a yellow solid. LCMS (ESI⁺): m/z (M+H⁺)=150.1. ¹H NMR: (400 MHz, DMSO-d₆) δ 5.16 (s, 1H), 4.50 (br s, 2H), 3.73 (s, 2H), 2.70 (s, 2H), 0.78-0.72 (m, 2H), 0.72-0.66 (m, 2H).

### Intermediate 43. N-(5-Chloro-1-methyl-1H-pyrazol-4-yl)-4-(tributylstannyl)pyrimidin-2-amine

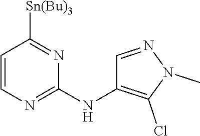

Step A: 4-Chloro-N-(5-chloro-1-methyl-1H-pyrazol-4-yl)pyrimidin-2-amine. A mixture of 4-chloro-2-(methylsulfonyl)pyrimidine (1.32 g, 6.85 mmol), 5-chloro-1-methyl-1H-pyrazol-4-amine (900 mg, 6.84 mmol) and THF (10 mL) was cooled to −50° C. To the mixture was added LiHMDS (14 mL, 14 mmol, 1 M in THF) dropwise and the resulting mixture was stirred at −50° C. for 5 h. The mixture was then warmed to 0° C. and water (5 mL) was added. The mixture was extracted with EtOAc and the organic extracts were dried over anhydrous Na₂SO₄, filtered, and concentrated. The residue was purified by FCC (0-60% ethyl acetate in petroleum ether) to afford 4-chloro-N-(5-chloro-1-methyl-1H-pyrazol-4-yl)pyrimidin-2-amine as a pale yellow solid (680 mg, 24%). LCMS (ESI⁺): mass calcd. for C₈H₇Cl₂N₅ 243.0 m/z, found 243.8 [M+H]⁺. ¹H NMR (400 MHz, DMSO-d₆) δ 9.29 (s, 1H), 8.32 (d, J=5.2 Hz, 1H), 7.63 (s, 1H), 6.87 (d, J=5.2 Hz, 1H), 3.79 (s, 3H).

Step B: N-(5-Chloro-1-methyl-1H-pyrazol-4-yl)-4-(tributylstannyl)pyrimidin-2-amine. A mixture of 4-chloro-N-(5-chloro-1-methyl-1H-pyrazol-4-yl)pyrimidin-2-amine (300 mg, 1.23 mmol), LiCl (315 mg, 7.43 mmol), 1,4-dioxane (5 mL), hexa-n-butylditin (1.25 mL, 2.47 mmol), Pd₂(dba)₃ (55 mg, 0.060 mmol) and tricyclohexylphosphine (40 mg, 0.143 mmol) was heated to reflux and stirred for 16 h. The mixture was cooled to room temperature and diluted with 50 mL EtOAc and filtered. The filter cake was washed with another 20 mL EtOAc. The filtrate was then collected, washed with brine, dried over anhydrous Na₂SO₄, filtered and concentrated. The residue was then purified by FCC (0-20% ethyl acetate in petroleum ether) to afford N-(5-chloro-1-methyl-1H-pyrazol-4-yl)-4-(tributylstannyl)pyrimidin-2-amine as a yellow oil (220 mg, 34%). LCMS (ESI⁺): mass calcd. for C₂₀H₃₄ClN₅Sn 499.15 m/z, found 500.1 [M+H]⁺.

### Intermediate 44: 3-(1-(6-Bromopyridin-2-yl)-1H-imidazol-4-yl)-3-hydroxy-1-methylpyrrolidin-2-one

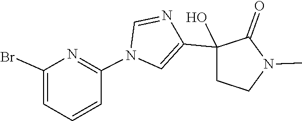 [SMILES: CN1CCC(O)(c2cn(-c3cccc(Br)n3)cn2)C1=O]

Step A. 3-Hydroxy-1-methyl-3-(1-trityl-1H-imidazol-4-yl)pyrrolidin-2-one. In an oven-dried and argon purged flask, 4-iodo-1-trityl-1H-imidazole (8.4 g, 19.2 mmol) was dissolved in 80 mL THF. The solution was then cooled to 0° C. using an ice water bath. To the flask was then added EtMgBr (7.0 mL, 21 mmol, 3 M in diethyl ether) via a syringe dropwise. The resulting mixture was allowed to stir for 1 h at 0° C. Then a solution of 1-methylpyrrolidine-2,3-dione (2.1 g, 18.6 mmol) in 20 mL of THF was added dropwise. The mixture was then slowly warmed to 25° C. over 1 h. The mixture was cooled to 0° C. and 20 mL sat. aq. NH₄Cl solution was added to the mixture to quench the remaining EtMgBr. The mixture was then diluted with 50 mL water and extracted with EtOAc (80 mL×3). The organic layers were collected, dried over anhydrous Na₂SO₄, filtered, and concentrated under reduced pressure to give a residue. The residue was subjected to flash column chromatography on silica gel (0-100% EtOAc in petroleum ether) to give 3-hydroxy-1-methyl-3-(1-trityl-1H-imidazol-4-yl)pyrrolidin-2-one (1.75 g, 22%) as a pink solid. ¹H NMR (400 MHz, CDCl₃) δ 7.39-7.29 (m, 10H), 7.18-7.07 (m, 6H), 6.94-6.90 (m, 1H), 3.56-3.47 (m, 1H), 3.46-3.41 (m, 1H), 3.41-3.33 (m, 1H), 2.91 (s, 3H), 2.70-2.65 (m, 1H), 2.42-2.26 (m, 1H). Mass calcd. for C₂₇H₂₅N₃O₂ 423.19 m/z, found 446.1 [M+Na]⁺.

Step B. 3-Hydroxy-3-(1H-imidazol-4-yl)-1-methylpyrrolidin-2-one trifluroacetate. To a solution of 3-hydroxy-1-methyl-3-(1-trityl-1H-imidazol-4-yl)pyrrolidin-2-one (500 mg, 1.2 mmol) dissolved in 10 mL DCM, TFA (1.75 mL, 23.6 mmol) was added. The resulting solution was allowed to stir at room temperature for 2 h. The mixture was concentrated under reduced pressure to give 3-hydroxy-3-(1H-imidazol-4-yl)-1-methylpyrrolidin-2-one trifluroacetate (350 mg) as a red solid. Mass calcd. for C₈H₁₁N₃O₂ 181.09 m/z, found 181.8 [M+H]⁺.

Step C. 3-(1-(6-Bromopyridin-2-yl)-1H-imidazol-4-yl)-3-hydroxy-1-methylpyrrolidin-2-one. A mixture of 3-hydroxy-3-(1H-imidazol-4-yl)-1-methylpyrrolidin-2-one trifluroacetate (350 mg, 0.48 mmol), 2-bromo-6-fluoropyridine (1.0 g, 6.0 mmol) and potassium carbonate (0.8 g, 6.0 mmol) was suspended in 4 mL DMSO. The resulting mixture was heated at 90° C. for 12 h. The mixture was cooled to room temperature, diluted with 20 mL water, and extracted with a 3:1 chloroform: isopropanol mixture (30 mL×3). The organic layers were collected, washed with water (20 mL×2) and brine (20 mL), dried over anhydrous sodium sulfate, filtered, and concentrated under reduced pressure. The product was subjected to flash column chromatography on silica gel (0%˜10% MeOH in EtOAc) to give 3-(1-(6-bromopyridin-2-yl)-1H-imidazol-4-yl)-3-hydroxy-1-methylpyrrolidin-2-one (220 mg, 40%) as a yellow oil. ¹H NMR (400 MHz, DMSO-d₆) δ 8.43 (d, J=1.6 Hz, 1H), 7.98-7.85 (m, 2H), 7.79 (d, J=1.6 Hz, 1H), 7.61 (d, J=7.6 Hz, 1H), 5.92 (s, 1H), 3.46-3.36 (m, 2H), 2.78 (s, 3H), 2.64-2.55 (m, 1H), 2.19-2.02 (m, 1H). Mass calcd. for C₁₃H₁₃BrN₄O₂ 336.0 m/z, found 338.7 [M+H (Br⁸¹)]⁺.

### Intermediate 45: 3-(3-(6-(2-((5-Chloro-1-methyl-1H-pyrazol-4-yl)amino)pyrimidin-4-yl)pyridin-2-yl)-1-((2-(trimethylsilyl)ethoxy)methyl)-1H-pyrazol-5-yl)-3-hydroxy-1-methylpyrrolidin-2-one

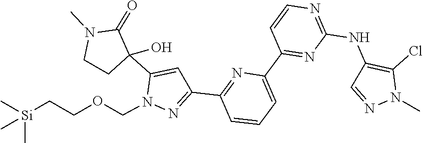

Step A. 4-(6-Bromopyridin-2-yl)-N-(5-chloro-1-methyl-1H-pyrazol-4-yl)pyrimidin-2-amine. A mixture of toluene (3 mL), N-(5-chloro-1-methyl-1H-pyrazol-4-yl)-4-(tributylstannyl)pyrimidin-2-amine (Intermediate 43, 170 mg, 0.341 mmol), 2,6-dibromopyridine (105 mg, 0.443 mmol), and TEA (95 μL, 0.68 mmol) was added to a flask. Then Pd(PPh₃)₄ (39 mg, 0.034 mmol) was added and the mixture was purged with N₂ for 5 min. The mixture was heated to 110° C. and stirred for 16 h. The reaction mixture was cooled to room temperature and then diluted with H₂O (10 mL), extracted with EtOAc (10 mL×3) and the combined organic layers were washed with brine (10 mL), dried over anhydrous Na₂SO₄, filtered, and concentrated in vacuo to give a brown oil. The oil was then subjected to FCC (0-30% EtOAc/pet ether) to give 4-(6-bromopyridin-2-yl)-N-(5-chloro-1-methyl-1H-pyrazol-4-yl)pyrimidin-2-amine as a yellow solid (60 mg, 44%). LCMS (ESI): RT=0.84 min, mass calcd. for C₁₃H₁₀BrClN₆ 363.98 m/z, found 367.0 [M+H]⁺.

Step B. N-(5-Chloro-1-methyl-1H-pyrazol-4-yl)-4-(6-(tributylstannyl)pyridin-2-yl)pyrimidin-2-amine. A mixture of 4-(6-bromopyridin-2-yl)-N-(5-chloro-1-methyl-1H-pyrazol-4-yl)pyrimidin-2-amine (60 mg, 0.16 mmol), LiCl (42 mg, 0.99 mmol) and 1,4-dioxane (2 mL) was charged with 1,1,1,2,2,2-hexabutyldistannane (340 mg, 0.59 mmol). The flask was evacuated and refilled with N₂ 3 times, and then treated with Pd₂(dba)₃ (8 mg, 0.008 mmol) and tricyclohexylphosphine (5 mg, 0.02 mmol). The reaction vessel was evacuated and refilled with N₂ 3 times and then stirred while heating for 16 hours at 120° C. The reaction mixture was cooled to room temperature and then treated with sat. aq. KF (10 mL) and stirred for 1 h at room temperature. The mixture was then extracted with EtOAc (10 mL×3) and the organic layer was washed with brine (10 mL), dried over anhydrous Na₂SO₄, filtered, and concentrated in vacuo to give a brown oil. The oil was then subjected to FCC (0-30% EtOAc/pet ether) to give N-(5-chloro-1-methyl-1H-pyrazol-4-yl)-4-(6-(tributylstannyl)pyridin-2-yl)pyrimidin-2-amine as a yellow oil (80 mg, 75%). LCMS (ESI): RT=1.46 min, mass calcd. for C₂₅H₃₇ClN₆Sn 576.18 m/z, found 577.4 [M+H]⁺.

Step C. 3-(3-(6-(2-((5-Chloro-1-methyl-1H-pyrazol-4-yl)amino)pyrimidin-4-yl)pyridin-2-yl)-1-((2-(trimethylsilyl)ethoxy)methyl)-1H-pyrazol-5-yl)-3-hydroxy-1-methylpyrrolidin-2-one. A mixture of toluene (3 mL), N-(5-chloro-1-methyl-1H-pyrazol-4-yl)-4-(6-(tributylstannyl)pyridin-2-yl)pyrimidin-2-amine (250 mg, 0.43 mmol), 3-(3-bromo-1-((2-(trimethylsilyl)ethoxy)methyl)-1H-pyrazol-5-yl)-3-hydroxy-1-methylpyrrolidin-2-one (Intermediate 29, 169 mg, 0.43 mmol), and TEA (125 μL, 0.9 mmol) were added to a flask. Then Pd(PPh₃)₄ (50 mg, 0.043 mmol) was added and the flask was evacuated and refilled with N₂ 3 times. The mixture was heated to 110° C. and stirred for 16 h. The mixture was cooled to room temperature, diluted with H₂O (10 mL) and extracted with EtOAc (10 mL×3). The combined organic layers were washed with brine (10 mL), dried over anhydrous Na₂SO₄, filtered, and concentrated in vacuo to give a brown oil. The oil was then subjected to FCC (0-100% EtOAc/pet ether) to give 3-(3-(6-(2-((5-chloro-1-methyl-1H-pyrazol-4-yl)amino)pyrimidin-4-yl)pyridin-2-yl)-1-((2-(trimethylsilyl)ethoxy)methyl)-1H-pyrazol-5-yl)-3-hydroxy-1-methylpyrrolidin-2-one as a yellow oil (130 mg, 49%). LCMS (ESI): RT=0.85 min, mass calcd. for C₂₇H₃₄ClN₉O₃Si 595.2 m/z, found 596.4 [M+H]⁺.

### Intermediate 46: N-(2-Methylpyridin-3-yl)-4-(tributylstannyl)pyrimidin-2-amine

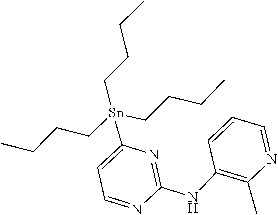 [SMILES: CCC[CH2][Sn]([CH2]CCC)([CH2]CCC)[c]1ccnc(Nc2cccnc2C)n1]

N-(2-Methylpyridin-3-yl)-4-(tributylstannyl)pyrimidin-2-amine was prepared in a manner analogous to Intermediate 43 using 2-methylpyridin-3-amine in place of 5-chloro-1-methyl-1H-pyrazol-4-amine. ¹H NMR (400 MHz, CDCl₃) δ 8.63-8.56 (m, 1H), 8.21-8.16 (m, 2H), 7.18-7.12 (m, 1H), 6.94-6.88 (m, 1H), 6.87-6.79 (m, 1H), 2.60 (s, 3H), 1.64-1.50 (m, 6H), 1.38-1.30 (m, 6H), 1.19-1.05 (m, 6H), 0.92-0.87 (m, 9H).

### Intermediate 47: N-(2-Methylpyridin-4-yl)-4-(tributylstannyl)pyrimidin-2-amine

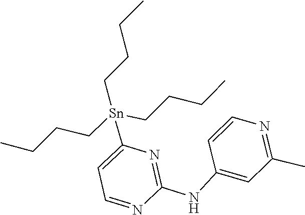 [SMILES: CCC[CH2][Sn]([CH2]CCC)([CH2]CCC)[c]1ccnc(Nc2ccnc(C)c2)n1]

N-(2-Methylpyridin-4-yl)-4-(tributylstannyl)pyrimidin-2-amine was prepared in a manner analogous to Intermediate 43 using 2-methylpyridin-4-amine in place of 5-chloro-1-methyl-1H-pyrazol-4-amine. LC-MS (ESI): Mass calcd. for C₂₂H₃₆N₄Sn 476.2 m/z found 477.1 [M+H]⁺. ¹H NMR (400 MHz, DMSO-d₆) δ 9.85 (s, 1H), 8.32 (d, J=4.6 Hz, 1H), 8.17 (d, J=5.7 Hz, 1H), 7.80 (d, J=1.8 Hz, 1H), 7.54 (dd, J=2.0, 5.7 Hz, 1H), 7.03 (d, J=4.6 Hz, 1H), 2.38 (s, 3H), 1.61-1.43 (m, 6H), 1.28 (sxt, J=7.3 Hz, 6H), 1.17-1.02 (m, 6H), 0.89-0.77 (m, 9H).

### Intermediate 48: (R)-3-(1-(6-Bromopyridin-2-yl)-1H-1,2,3-triazol-4-yl)-4,4-difluoro-3-hydroxy-1-methylpyrrolidin-2-one

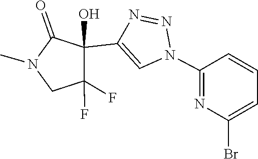 [SMILES: CN1CC(F)(F)[C@@](O)(c2cn(-c3cccc(Br)n3)nn2)C1=O]

(R)-3-(1-(6-Bromopyridin-2-yl)-1H-1,2,3-triazol-4-yl)-4,4-difluoro-3-hydroxy-1-methylpyrrolidin-2-one was prepared in a manner analogous to (R)-3-(1-(6-bromopyridin-2-yl)-1H-1,2,3-triazol-4-yl)-3-hydroxy-1-methylpyrrolidin-2-one (Intermediate 51) using (R)-3-ethynyl-4,4-difluoro-3-hydroxy-1-methylpyrrolidin-2-one (Intermediate 2) in place of (R)-3-ethynyl-3-hydroxy-1-methylpyrrolidin-2-one. ¹H NMR (400 MHz, DMSO-d₆) δ 8.70 (s, 1H), 8.17-8.04 (m, 2H), 7.84 (d, J=7.7 Hz, 1H), 7.44 (s, 1H), 4.02-3.89 (m, 2H), 2.95 (s, 3H), 1.99 (s, 1H).

### Intermediate 49. 2-Chloropyrimidine-4,6-d₂

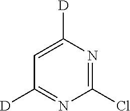

Step A. Pyrimidin-4,6-d₂-2-amine. To a solution of CD₃OD (10 mL, 246 mmol) in THF (30 mL) were added 4,6-dichloropyrimidin-2-amine (10 g, 61 mmol), TEA (18.5 g, 183 mmol) and 10% Pd/C (1.95 g, 1.83 mmol) sequentially. The reaction flask was evacuated and refilled with D₂ three times and then the mixture was stirred at room temperature for 12 h under D₂. The above reaction sequence was repeated 7 more times and the resulting reaction mixtures were combined and filtered. The filtrate was concentrated to afford a white solid. The white solid was dissolved in EtOAc (700 mL) and stirred at room temperature for 12 hrs. The mixture was filtered and the filtrate was concentrated down to 100 mL. Then, heptane (500 mL) was added and the resulting mixture was concentrated down to 200 mL. The mixture was filtered and the filtrate was concentrated to provide the title compound as a white solid (33.7 g, 71%). ¹H NMR (400 MHz, DMSO-d₆) δ 6.61-6.45 (m, 3H).

Step B. 2-Chloropyrimidine-4,6-d₂. A mixture of pyrimidin-4,6-d₂-2-amine (33.7 g, 347 mmol) in HCl (342 g, 3.47 mol, 37% solution in water) was cooled to 0° C. Then, NaNO₂ (47.9 g, 694 mmol) and water (337 mL) were added and the resulting mixture was warmed to room temperature and stirred at room temperature for 16 h. The mixture was cooled to 0° C. and then the pH of the mixture was adjusted to pH 7-8 by the addition of 30% aqueous NaOH. The mixture was extracted with MTBE (3×600 mL). The combined organic layers were dried over anhydrous Na₂SO₄, filtered and concentrated to provide the title compound as a yellow solid (13.4 g, 33%). ¹H NMR (400 MHz, DMSO-d₆) δ 7.59 (br s, 1H).

### Intermediate 50. (2-Chloropyrimidin-4-yl-6-d)zinc(II) chloride

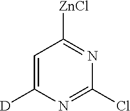

A solution of 2-chloropyrimidine-4,6-d₂ (230.3 mL, 230.3 mmol, 1 M in THF, Intermediate 49) in THF (280 mL) was cooled to −25° C. Then, TMP-MgCl·LiCl (24.4 g, 209 mmol) was added and the resulting mixture was stirred at −25° C. for 2 hrs. After that time, ZnCl₂ (314 mL, 314 mmol, 1 M in THF) was added, the mixture was warmed to room temperature, and stirred at room temperature for 12 hrs. The reaction mixture was used in the next step directly.

### Intermediate 51. (R)-3-(1-(6-Bromopyridin-2-yl)-1H-1,2,3-triazol-4-yl)-3-hydroxy-1-methylpyrrolidin-2-one

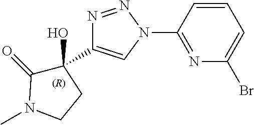

Step A. 2-Azido-6-bromopyridine. A mixture of 2-bromo-6-hydrazineylpyridine (90.0 g, 479 mmol) in Et₂O (288 mL) and water (400 mL) was cooled to 0° C. Then, HCl (183.6 g, 5.036 mol), NaNO₂ (39.6 g, 574 mmol) and water (180 mL) were added sequentially. The resulting mixture was warmed to room temperature and then stirred at room temperature for 2 hrs. The reaction was filtered and the filtrate was concentrated to provide the title compound as a brown solid (103 g) which was used without further purification. ¹H NMR (400 MHz, CDCl₃) δ 7.40 (t, J=8.00 Hz, 1H), 7.15 (d, J=8.00 Hz, 1H), 6.68 (d, J=8.00 Hz, 1H).

Step B. (R)-3-(1-(6-Bromopyridin-2-yl)-1H-1,2,3-triazol-4-yl)-3-hydroxy-1-methylpyrrolidin-2-one. A mixture of CuSO₄·5H₂O (40.8 g, 163 mmol) and sodium ascorbate (57.5 g, 653 mmol) in water (780 mL) and t-BuOH (780 mL) was stirred at room temperature for 10 min. Then, (R)-3-ethynyl-3-hydroxy-1-methylpyrrolidin-2-one (45.45 g, 326.6 mmol) and NaH₂PO₄ (195.9 g, 1.630 mol) were added sequentially and the resulting mixture was stirred at room temperature for 10 min. 2-Azido-6-bromopyridine (78.0 g, 392 mmol) was then added and the reaction mixture was stirred at room temperature for 2 hrs. After that time, the reaction mixture was poured into water (1 L) and the mixture was stirred at room temperature for 30 min. The mixture was extracted with ACN/EtOAc (1/2, 3×500 mL), and the combined organic layers were concentrated. MTBE (600 mL) was added to the residue and the mixture was stirred at room temperature for 2 hrs. The suspension was filtered and the filtrate was concentrated to afford a yellow solid. The solid was purified by silica gel chromatography (0-10% MeOH/DCM) to provide the title compound as a white solid (54.7 g, 48%). 1H NMR (400 MHz, DMSO-d₆) δ 8.62 (s, 1H), 8.16 (d, J=7.60 Hz, 1H), 8.00-8.10 (m, 1H), 7.82 (d, J=7.60 Hz, 1H), 6.33 (s, 1H), 3.39-3.53 (m, 2H), 2.81 (s, 3H), 2.72-2.76 (m, 1H), 2.19-2.34 (m, 1H). MS (ESI⁺): m/z=337.9.

### Intermediate 52. (R)-3-(5-(6-Bromopyridin-2-yl)isoxazol-3-yl)-3-hydroxy-1-methylpyrrolidin-2-one

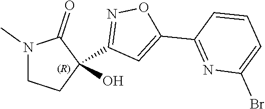

Step A. (3R)-3-(3-(6-Bromopyridin-2-yl)-3-hydroxyprop-1-yn-1-yl)-3-hydroxy-1-methylpyrrolidin-2-one. A solution of (R)-3-ethynyl-3-hydroxy-1-methylpyrrolidin-2-one (700 g, 5.03 mol) in THF (14 L) was cooled to −35° C. and then iPrMgCl·LiCl (9.56 L, 1 M in THF) was added dropwise. The resulting mixture was stirred at −35° C. for 1 h, then a solution of 6-bromopicolinaldehyde (1.22 kg, 6.54 mol) in THF (5 L) was added dropwise. The mixture was then allowed to warm to −30° C. and stirred at −30° C. for 1 h. The mixture was warmed to 5° C., then water (10 L) was added and the mixture was extracted with EtOAc (4×5 L). The combined organic layers were washed with brine (500 mL), dried over anhydrous Na₂SO₄, filtered and concentrated. The residue was triturated with MTBE (2 L) at 25° C. for 30 min to provide the title compound as a yellow solid (850 g, 44%). ¹H NMR (400 MHz, CDCl₃) δ 7.58-7.48 (m, 2H), 7.32-7.32 (m, 1H), 7.42-7.32 (m, 1H), 5.46 (d, J=1.88 Hz, 1H), 3.42-3.23 (m, 2H), 2.87-2.80 (m, 3H), 2.54-2.39 (m, 1H), 2.22 (dt, J=12.88, 7.94 Hz, 1H). MS (ESI⁺): m/z=324.9.

Step B. (R)-3-(3-(6-Bromopyridin-2-yl)-3-oxoprop-1-yn-1-yl)-3-hydroxy-1-methylpyrrolidin-2-one. To a solution of (3R)-3-(3-(6-bromopyridin-2-yl)-3-hydroxyprop-1-yn-1-yl)-3-hydroxy-1-methylpyrrolidin-2-one (850 g, 2.20 mol) in DCM (30 L) was added MnO₂ (2.27 kg, 26.1 mol), and the resulting mixture was heated at 30° C. for 12 hrs. The reaction mixture was filtered and the filtrate was concentrated. The residue was triturated with MTBE (2 L) at 25° C. for 1 h to provide the title compound as a yellow solid (750 g, 89%). ¹H NMR (400 MHz, CDCl₃) δ 8.06 (br s, 1H), 7.70 (br s, 2H), 4.22-3.92 (m, 1H), 3.76 (br s, 2H), 3.45 (br s, 1H), 3.06-2.90 (m, 3H), 2.75 (br s, 1H), 2.41 (br s, 1H). MS (ESI⁺): m/z=323.

Step C. (3R)-3-(5-(6-Bromopyridin-2-yl)-5-hydroxy-4,5-dihydroisoxazol-3-yl)-3-hydroxy-1-methylpyrrolidin-2-one. To a solution of (R)-3-(3-(6-bromopyridin-2-yl)-3-oxoprop-1-yn-1-yl)-3-hydroxy-1-methylpyrrolidin-2-one (800 g, 2.48 mol) in THF (7 L) at 0° C. was added NH₂OH·H₂O (1.31 kg, 19.8 mol). The resulting mixture was stirred at room temperature for 2 hrs, then quenched by the addition of ice-water (8 L). EtOAc (2 L) was added, the layers separated, and the aqueous layer further extracted with EtOAc (3×5 L). The combined organic layers were washed with brine (5 L), dried over anhydrous Na₂SO₄, filtered and concentrated to provide the title compound as a yellow solid (800 g, 91%). MS (ESI⁺): m/z=356.

Step D. (R)-3-(5-(6-Bromopyridin-2-yl)isoxazol-3-yl)-3-hydroxy-1-methylpyrrolidin-2-one. To a solution of (3R)-3-(5-(6-bromopyridin-2-yl)-5-hydroxy-4,5-dihydroisoxazol-3-yl)-3-hydroxy-1-methylpyrrolidin-2-one (400 g, 1.12 mol) in THF (2.4 L) was added TsOH·H₂O (641 g, 3.37 mol) and the resulting mixture was heated at 50° C. for 12 hrs. The reaction was cooled to 5° C., then ice-water (2 L) was added and the mixture diluted with EtOAc (2 L). The layers were separated and the aqueous layer was further extracted with EtOAc (3×2 L). The combined organic layers were washed with brine (500 mL), dried over anhydrous Na₂SO₄, filtered and concentrated. The residue was purified by silica gel chromatography (10% THF/DCM, isocratic) to provide the title compound as a yellow solid (450 g, 57%). ¹H NMR (400 MHz, DMSO-d₆) δ 8.04-7.98 (m, 1H), 7.94 (t, J=7.75 Hz, 1H), 7.77 (d, J=7.88 Hz, 1H), 7.19 (s, 1H), 6.57 (s, 1H), 3.49-3.36 (m, 2H), 2.80 (s, 3H), 2.73-2.63 (m, 1H), 2.30-2.18 (m, 1H). MS (ESI⁺): m/z=338.

### Intermediate 53. (R)-3-(3-(6-(2-Chloropyrimidin-4-yl-6-d)pyridin-2-yl)isoxazol-5-yl)-3-hydroxy-1-methylpyrrolidin-2-one

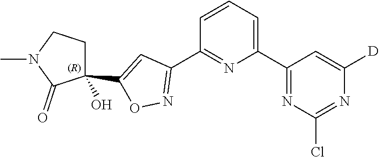

A flask containing TMP·MgCl—LiCl (215 mL, 215 mmol, 1 M in THF/toluene) was cooled to −10 to −25° C. and then a solution of 2-chloropyrimidine-4,6-d₂ (22.4 g, 192 mmol, Intermediate 49) in THF (220 mL) was added dropwise. The resulting mixture (mixture 1) was stirred at −10 to −25° C. for 2 hrs, then ZnCl₂ (288 mL, 288 mmol, 1 M in THF) was added and the mixture was stirred at room temperature for 12 hrs. To a separate flask was added THF (176 mL), (R)-3-(3-(6-bromopyridin-2-yl)isoxazol-5-yl)-3-hydroxy-1-methylpyrrolidin-2-one (25.6 g, 75.7 mmol, Intermediate 13 Step A) and Pd(PPh₃)₄ (8.66 g, 7.50 mmol) sequentially and the resulting mixture (mixture 2) was heated to 60° C. Then, mixture 1 was added to mixture 2 dropwise and the resulting mixture was heated at 60° C. for 1 h. The reaction was cooled to room temperature, poured into saturated aqueous NH₄Cl (900 mL) and extracted with EtOAc (1 L×5). The combined organic layers were washed with brine (1.5 L), dried over anhydrous Na₂SO₄, filtered and concentrated. The residue was triturated with MeOH (120 mL) at 25° C. for 12 hrs, then filtered and the filtrate was concentrated to provide the title compound as a yellow solid (19.5 g, 27%). ¹H NMR (400 MHz, DMSO-d₆) δ 8.56 (s, 1H), 8.48 (dd, J=2.90, 5.80 Hz, 1H), 8.25-8.18 (m, 2H), 7.22 (s, 1H), 6.78 (s, 1H), 3.47 (s, 2H), 2.85 (s, 3H), 2.62 (s, 1H), 2.30 (s, 1H). MS (ESI⁺): m/z=373.0.

### Intermediate 54. (*S)-4-Methyl-6,7-dihydro-4H-pyrazolo[5,1-c][1,4]oxazin-3-amine

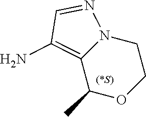

(*S)-4-Methyl-6,7-dihydro-4H-pyrazolo[5,1-c][1,4]oxazin-3-amine was prepared as described in Intermediate 70 and eluted as the first peak during chiral SFC purification to provide the title compound (3.1 g, 90%) as a brown oil. MS (ESI⁺): m/z=154.0.

### Intermediate 55. 5-Methyl-N-(1-methyl-1H-pyrazol-4-yl)-4-(trimethylstannyl)pyrimidin-2-amine

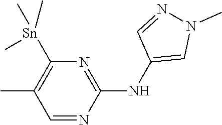

Step A. 4-Chloro-5-methyl-2-(methylsulfonyl)pyrimidine. To a mixture of 4-chloro-5-methyl-2-(methylthio)pyrimidine (5.00 g, 28.6 mmol) in THF (30 mL) and acetone (30 mL) was added potassium peroxymonosulfate (38.6 g, 62.8 mmol) in water (30 mL) and the resulting mixture was stirred at room temperature for 16 hrs. The reaction mixture was diluted with EtOAc (200 mL) and washed with water (2×100 mL). The organic layer was dried over anhydrous Na₂SO₄, filtered and concentrated. The residue was purified by silica gel chromatography (0-50% EtOAc/petroleum ether) to provide the title compound as a white solid (3.9 g, 64%). MS (ESI⁺): m/z=206.8.

Step B. 4-Chloro-5-methyl-N-(1-methyl-1H-pyrazol-4-yl)pyrimidin-2-amine. A solution of 1-methyl-1H-pyrazol-4-amine (2.4 g, 25 mmol) in THF (30 mL) was cooled to −78° C., then LDA (15 mL, 30 mmol, 2 M in THF) was added portion-wise over 5 min. The resulting mixture was stirred at −78° C. for 1 h, then a solution of 4-chloro-5-methyl-2-(methylsulfonyl)pyrimidine (3.00 g, 14.5 mmol) in THF (5 mL) was added. The mixture was stirred at −78° C. for 1 h, and was then warmed to room temperature and stirred at room temperature for 16 hrs. The reaction mixture was diluted with water (200 mL) and extracted with DCM (3×100 mL). The combined organic layers were washed with brine (150 mL), dried over anhydrous Na₂SO₄, filtered and concentrated. The residue was purified by silica gel chromatography (0-80% EtOAc/petroleum ether) to provide the title compound as a yellow solid (700 mg, 21%). MS (ESI⁺): m/z=223.8.

Step C. 5-Methyl-N-(1-methyl-1H-pyrazol-4-yl)-4-(trimethylstannyl)pyrimidin-2-amine. To a solution of 4-chloro-5-methyl-N-(1-methyl-1H-pyrazol-4-yl)pyrimidin-2-amine (800 mg, 3.58 mmol) in 1,4-dioxane (15 mL) were added (SnMe₃)₂ (1.91 g, 5.83 mmol) and Pd(P(Cy)₃)₂Cl₂ (240 mg, 0.325 mmol) and the resulting mixture was purged with Ar three times and then heated at 105° C. for 16 hrs. After that time, saturated aqueous KF (50 mL) was added and the mixture was stirred at room temperature for 1 h. The precipitate was removed by filtration and the filtrate extracted with EtOAc (3×50 mL). The combined organic layers were washed with brine, dried over anhydrous Na₂SO₄, filtered and concentrated. The residue was purified sequentially by silica gel chromatography (0-100% EtOAc/petroleum ether) and then reverse-phase HPLC (Xtimate C18 column, 10 μm, 40×150 mm; 40-70% ACN/H₂O (with 0.05% NH₃ and 10 mM NH₄HCO₃)) to afford the title compound as a red-white solid (270 mg, 21%). MS (ESI⁺): m/z=354.1.

### Intermediate 56. (R)-3-(3-(6-Chloropyridin-2-yl)isoxazol-5-yl)-3-hydroxy-1-methylpyrrolidin-2-one

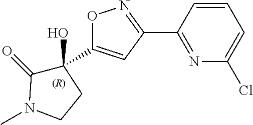

To a solution of (R)-3-ethynyl-3-hydroxy-1-methylpyrrolidin-2-one (304 mg, 2.18 mmol) in DCM (7 mL) were added (Z)-6-chloro-N-hydroxypicolinimidoyl chloride (500 mg, 2.62 mmol) and TEA (0.91 mL, 6.54 mmol), and the resulting mixture was stirred at 25° C. for 16 hrs. The mixture was diluted with DCM (30 mL), washed sequentially with 1 N aqueous HCl (30 mL), saturated aqueous NaHCO₃ (30 mL) and brine (30 mL), dried over anhydrous Na₂SO₄, filtered and concentrated. The residue was purified by silica gel chromatography (0-100% EtOAc/petroleum ether) to afford the title compound as a white solid (440 mg, 69%). MS (ESI⁺): m/z=294.1.

### Intermediate 57. 4-Methyl-N-(1-methyl-1H-pyrazol-4-yl)-6-(tributylstannyl)pyrimidin-2-amine

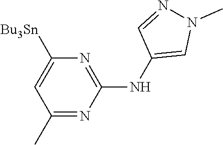

Step A. 4-Chloro-6-methyl-2-(methylsulfonyl)pyrimidine. The title compound (1 g, 83%) was prepared using conditions analogous to those described in Intermediate 55, Step A using 4-chloro-6-methyl-2-(methylthio)pyrimidine in place of 4-chloro-5-methyl-2-(methylthio)pyrimidine. MS (ESI⁺): m/z=207.0.

Step B. 4-Chloro-6-methyl-N-(1-methyl-1H-pyrazol-4-yl)pyrimidin-2-amine. The title compound (810 mg, 77%) was prepared using conditions analogous to those described in Intermediate 55, Step B using 4-chloro-6-methyl-2-(methylsulfonyl)pyrimidine (Step A) in place of 4-chloro-5-methyl-N-(1-methyl-1H-pyrazol-4-yl)pyrimidin-2-amine. MS (ESI⁺): m/z=224.1.

Step C. 4-Methyl-N-(1-methyl-1H-pyrazol-4-yl)-6-(tributylstannyl)pyrimidin-2-amine. To a solution of 4-chloro-6-methyl-N-(1-methyl-1H-pyrazol-4-yl)pyrimidin-2-amine (750 mg, 3.35 mmol) in 1,4-dioxane (15 mL) were added Pd₂(dba)₃ (330 mg, 0.36 mmol), P(Cy)₃ (105 mg, 0.374 mmol), LiCl (900 mg, 21.2 mmol) and (SnBu₃)₂ (2.54 g, 4.38 mmol). The resulting mixture was purged with Ar three times and then heated at 120° C. for 10 hrs. After that time, saturated aqueous KF (30 mL) was added and the mixture was stirred at room temperature for 1 h. The precipitate was removed by filtration and the filtrate extracted with EtOAc (3×30 mL). The combined organic layers were washed with brine, dried over anhydrous Na₂SO₄, filtered and concentrated. The residue was purified by silica gel chromatography (0-50% EtOAc/petroleum ether) to afford the title compound as a yellow oil (770 mg, 45%). MS (ESI⁺): m/z=480.1.

### Intermediate 58. (R)-2-amino-1-(3,3-difluoroazetidin-1-yl)propan-1-one

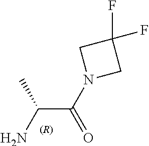

Step A. tert-Butyl (R)-(1-(3,3-difluoroazetidin-1-yl)-1-oxopropan-2-yl)carbamate. A mixture of (R)-2-((tert-butoxycarbonyl)amino)propanoic acid (2.00 g, 10.6 mmol), DIEA (3.80 mL, 21.4 mmol) and HBTU (6.00 g, 15.8 mmol) in DMF (15 mL) was stirred at room temperature for 15 min. Then, 3,3-difluoroazetidine (1.8 g, 14 mmol) was added and the resulting mixture was stirred at room temperature for 1.5 hrs. The mixture was diluted with water (50 mL) and extracted with EtOAc (3×70 mL). The combined organic layers were washed with brine (40 mL), dried over anhydrous Na₂SO₄, filtered and concentrated. The residue was purified by reverse-phase HPLC (Xtimate C18 column, 10 μm, 40×150 mm; 20-50% ACN/H₂O (with 0.05% aqueous NH₃ and 10 mM NH₄HCO₃)) to afford the title compound as a yellow solid (2.46 g, 88%). ¹H NMR (400 MHz, DMSO-d₆) δ 7.22-7.14 (m, 1H), 4.73-4.54 (m, 2H), 4.37-4.21 (m, 2H), 4.08-3.98 (m, 1H), 1.38 (s, 9H), 1.18-1.11 (m, 3H).

Step B. (R)-2-amino-1-(3,3-difluoroazetidin-1-yl)propan-1-one. A mixture of tert-butyl (R)-(1-(3,3-difluoroazetidin-1-yl)-1-oxopropan-2-yl)carbamate (2.44 g, 9.23 mmol) and TFA (7.0 mL, 94 mmol) in DCM (15 mL) was stirred at room temperature for 1.5 hrs, then concentrated to afford the title compound as a yellow oil (3.9 g) which was used without further purification. ¹H NMR (400 MHz, DMSO-d₆) δ 8.26-8.19 (m, 2H), 4.89-4.63 (m, 2H), 4.48-4.33 (m, 2H), 4.12-3.98 (m, 1H), 1.39-1.27 (m, 3H).

### Intermediate 59. (S)-2-Amino-1-(3,3-difluoroazetidin-1-yl)propan-1-one trifluoroacetate

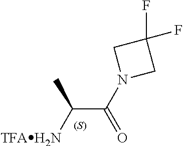

The title compound (1 g, 100%) was prepared using conditions analogous to those described in Intermediate 58 using (S)-2-((tert-butoxycarbonyl)amino)propanoic acid in place of (R)-2-((tert-butoxycarbonyl)amino)propanoic acid in Step A. MS (ESI⁺): m/z=164.8.

### Intermediate 60. (S)-2-Amino-1-(3-(trifluoromethyl)azetidin-1-yl)propan-1-one trifluoroacetate

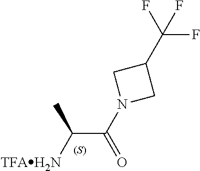

The title compound (800 mg) was prepared using conditions analogous to those described in Intermediate 58 using (S)-2-((tert-butoxycarbonyl)amino)propanoic acid in place of (R)-2-((tert-butoxycarbonyl)amino)propanoic acid and 3-(trifluoromethyl)azetidine hydrochloride in place of 3,3-difluoroazetidine in Step A, and was used without further purification. MS (ESI⁺): m/z=196.8.

### Intermediate 61. (R)-2-Amino-1-(3-fluoro-3-methylazetidin-1-yl)propan-1-one

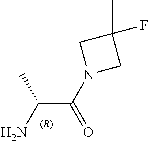

Step A. tert-Butyl (R)-(1-(3-fluoro-3-methylazetidin-1-yl)-1-oxopropan-2-yl)carbamate. To a solution of (R)-2-((tert-butoxycarbonyl)amino)propanoic acid (1.00 g, 5.29 mmol) in DMF (15 mL) were added DIEA (4.4 mL, 27 mmol), HATU (2.4 g, 6.3 mmol) and 3-fluoro-3-methylazetidine hydrochloride (795 mg, 6.33 mmol) and the resulting mixture was stirred at room temperature for 16 hrs. The mixture was diluted with EtOAc (50 mL) and washed with water (2×30 mL). The organic layer was dried over anhydrous Na₂SO₄, filtered and concentrated. The residue was purified by silica gel chromatography (0-50% EtOAc/petroleum ether) to afford the title compound as a colorless oil (1.23 g, 83%). MS (ESI⁺): m/z=260.9.

Step B. (R)-2-Amino-1-(3-fluoro-3-methylazetidin-1-yl)propan-1-one. A mixture of tert-butyl (R)-(1-(3-fluoro-3-methylazetidin-1-yl)-1-oxopropan-2-yl)carbamate (1.23 g, 4.73 mmol) and TFA (10.5 mL, 141 mmol) in DCM (18 mL) was stirred at room temperature for 6 hrs, then concentrated to afford the title compound as a yellow oil (1.8 g), which was used without further purification. MS (ESI⁺): m/z=160.8.

### Intermediate 62. (S)-2-Amino-1-(3-fluoro-3-methylazetidin-1-yl)propan-1-one

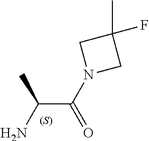

Step A. tert-Butyl (S)-(1-(3-fluoro-3-methylazetidin-1-yl)-1-oxopropan-2-yl)carbamate. To a solution of (S)-2-((tert-butoxycarbonyl)amino)propanoic acid (1.00 g, 5.29 mmol) in DMF (12 mL) were added DIEA (4.6 mL, 27 mmol), HATU (2.4 g, 6.3 mmol) and 3-fluoro-3-methylazetidine hydrochloride (664 mg, 5.29 mmol) and the resulting mixture was stirred at room temperature for 16 hrs. The mixture was diluted with water (20 mL) and extracted with EtOAc (3×20 mL). The combined organic layers were washed with brine (20 mL), dried over anhydrous Na₂SO₄, filtered and concentrated. The residue was purified by reverse-phase HPLC (Xtimate C18 column, 10 μm, 40×150 mm; 20-50% ACN/H₂O (with 0.05% aqueous NH₃ and 10 mM NH₄HCO₃)) to afford the title compound as a red oil (1.15 g, 84%). MS (ESI⁺): m/z=161.0.

Step B. (S)-2-Amino-1-(3-fluoro-3-methylazetidin-1-yl)propan-1-one. A mixture of tert-butyl (S)-(1-(3-fluoro-3-methylazetidin-1-yl)-1-oxopropan-2-yl)carbamate (1.1 g, 4.2 mmol) and TFA (3.14 mL, 42.3 mmol) in DCM (10 mL) was stirred at room temperature for 2 hrs, then concentrated to afford the title compound as a red oil (1.13 g, 98%). MS (ESI⁺): m/z=161.1.

### Intermediate 63. (S)-2-amino-N-methyl-N-phenylpropanamide trifluoroacetate

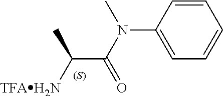

The title compound (1.1 g, 94%) was prepared using conditions analogous to those described in Intermediate 62 using N-methylaniline in place of 3-fluoro-3-methylazetidine hydrochloride in Step A. MS (ESI⁺): m/z=179.1.

### Intermediate 64. 2-Amino-1-(3,3-difluoroazetidin-1-yl)ethan-1-one hydrochloride

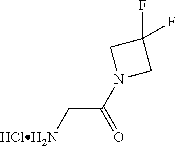

Step A. tert-Butyl (2-(3,3-difluoroazetidin-1-yl)-2-oxoethyl)carbamate. A mixture of 2-((tert-butoxycarbonyl)amino)acetic acid (1.00 g, 5.71 mmol), 3,3-difluoroazetidine hydrochloride (739 mg, 5.7 mmol), DIEA (5.00 mL, 28.7 mmol) and HATU (4.34 g, 11.4 mmol) in DCM (40 mL) was stirred at room temperature for 16 hrs. Then, the mixture was poured into water (10 mL) and extracted with DCM (3×15 mL). The combined organic layers were dried over anhydrous Na₂SO₄, filtered and concentrated. The residue was purified by silica gel chromatography (0-40% EtOAc/petroleum ether) to provide the title compound as a brown solid (1.3 g, 91%). ¹H NMR (400 MHz, DMSO-d₆) δ 7.04 (t, J=5.8 Hz, 1H), 4.62 (t, J=12.6 Hz, 2H), 4.30 (t, J=12.6 Hz, 2H), 3.63 (d, J=6.0 Hz, 2H), 1.39 (s, 9H).

Step B. 2-Amino-1-(3,3-difluoroazetidin-1-yl)ethan-1-one hydrochloride. A mixture of tert-butyl (2-(3,3-difluoroazetidin-1-yl)-2-oxoethyl)carbamate (500 mg, 2 mmol) in HCl (5 mL, 20 mmol, 4 M in 1,4-dioxane) was stirred at room temperature for 2 hrs, then concentrated to afford the title compound as a brown solid (400 mg), which was used without further purification. ¹H NMR (400 MHz, DMSO-d₆) δ 8.44 (br. s, 3H), 4.72 (br. t, J=12.5 Hz, 2H), 4.39 (br. t, J=12.5 Hz, 2H), 3.70 (br. s, 2H).

### Intermediate 65. 3-(5-(6-Chloropyridin-2-yl)-1-methyl-1H-pyrazol-3-yl)-3-hydroxy-1-methylpyrrolidin-2-one

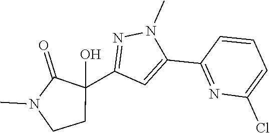 [SMILES: CN1CCC(O)(c2cc(-c3cccc(Cl)n3)n(C)n2)C1=O]

Step A. 2-(3-Bromo-1-methyl-1H-pyrazol-5-yl)-6-chloropyridine. To a mixture of 3,5-dibromo-1-methyl-1H-pyrazole (4.5 g, 19 mmol), 2-chloro-6-(4,4,5,5-tetramethyl-1,3,2-dioxaborolan-2-yl)pyridine (3.8 g, 16 mmol) and K₂CO₃ (7.8 g, 56 mmol) in 1,4-dioxane:H₂O (4:1, 120 mL) was added Pd(dppf)Cl₂ (1.4 g, 1.9 mmol). The mixture was sparged with Ar for 5 min, then heated at 105° C. for 16 hrs. The reaction mixture was allowed to slowly cool to room temperature, then was diluted with water (80 mL) and extracted with EtOAc (3×150 mL). The combined organic layers were washed with brine (100 mL), dried over anhydrous Na₂SO₄, filtered and concentrated. The residue was purified by silica gel chromatography (0-50% EtOAc/petroleum ether) to provide the title compound as a white solid (1.6 g, 31%). MS (ESI⁺): m/z=272.0.

Step B. 3-(5-(6-Chloropyridin-2-yl)-1-methyl-1H-pyrazol-3-yl)-3-hydroxy-1-methylpyrrolidin-2-one. To a flask was added 2-(3-bromo-1-methyl-1H-pyrazol-5-yl)-6-chloropyridine (700 mg, 2.57 mmol) in DCM (5 mL), and the flask was evacuated and backfilled with N₂ three times. The solution was cooled to −72° C. and n-BuLi (0.17 mL, 0.43 mmol, 2.5 M in hexanes) was added. The mixture was stirred at −72° C. for 30 min, then a solution of 1-methylpyrrolidine-2,3-dione (490 mg, 4.33 mmol) in DCM (2 mL) was added and the resulting mixture was stirred at −72° C. for 3 hrs. After that time, the reaction mixture was allowed to slowly warm to room temperature then diluted with water (5 mL) and extracted with DCM (3×10 mL). The combined organic layers were washed with brine (8 mL), dried over anhydrous Na₂SO₄, filtered and concentrated. The residue was purified by silica gel chromatography (0-100% EtOAc/petroleum ether) to provide the title compound as a white solid (330 mg, 39%). MS (ESI⁺): m/z=307.1.

### Intermediate 66. N-(1-(2,2-Difluoroethyl)-3-methoxy-1H-pyrazol-4-yl)-4-(tributylstannyl)pyrimidin-2-amine

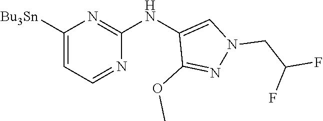

Step A. 4-Chloro-N-(1-(2,2-difluoroethyl)-3-methoxy-1H-pyrazol-4-yl)pyrimidin-2-amine. A mixture of 1-(2,2-difluoroethyl)-3-methoxy-1H-pyrazol-4-amine (300 mg, 1.69 mmol) and 4-chloro-2-(methylsulfonyl)pyrimidine (327 mg, 1.7 mmol) in THF (10 mL) was added to a flask which was subsequently evacuated and backfilled with N₂ three times, then cooled to −50° C. Then, LiHMDS (3.4 mL, 3.4 mmol, 1 M in THF) was added and the resulting mixture was stirred at −50° C. for 2 hrs. After that time, saturated aqueous NH₄Cl (10 mL) was added and the mixture was extracted with EtOAc (3×30 mL). The combined organic layers were washed with brine (20 mL), dried over anhydrous Na₂SO₄, filtered and concentrated. The residue was purified by silica gel chromatography (0-30% EtOAc/petroleum ether) to provide the title compound as a brown solid (200 mg, 96%). MS (ESI⁺): m/z=290.1.

Step B. N-(1-(2,2-Difluoroethyl)-3-methoxy-1H-pyrazol-4-yl)-4-(tributylstannyl)pyrimidin-2-amine. To a solution of 4-chloro-N-(1-(2,2-difluoroethyl)-3-methoxy-1H-pyrazol-4-yl)pyrimidin-2-amine (300 mg, 1.04 mmol) and LiCl (264 mg, 6.3 mmol) in 1,4-dioxane (10 mL) was added 1,1,1,2,2,2-hexabutyldistannane (1.44 g, 2.48 mmol) and the resulting mixture was purged with N₂ three times. Then, Pd₂(dba)₃ (48 mg, 0.052 mmol) and tricyclohexylphosphine (30 mg, 0.11 mmol) were added and the mixture was purged again with N₂ three times. The resulting mixture was heated at 120° C. for 12 hrs. After that time, saturated aqueous KF (10 mL) was added and the mixture was stirred at room temperature for 2 hrs. The mixture was diluted with water (15 mL) and extracted with EtOAc (3×30 mL). The combined organic layers were washed with brine (20 mL), dried over anhydrous Na₂SO₄, filtered and concentrated. The residue was purified by silica gel chromatography (0-30% EtOAc/petroleum ether) to afford the title compound as a brown solid (300 mg, 37%). MS (ESI⁺): m/z=546.2.

### Intermediate 67. N-(4-(Tributylstannyl)pyrimidin-2-yl)-5,6-dihydro-4H-pyrrolo[1,2-b]pyrazol-3-amine

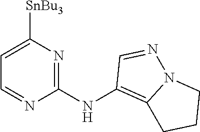

Step A. N-(4-Chloropyrimidin-2-yl)-5,6-dihydro-4H-pyrrolo[1,2-b]pyrazol-3-amine. A solution of 5,6-dihydro-4H-pyrrolo[1,2-b]pyrazol-3-amine (2.38 g, 19.3 mmol) in THF (70 mL) was cooled to −72° C. and then LiHMDS (36 mL, 36 mmol, 1 M in THF) was added. The resulting mixture was stirred at −72° C. for 30 min, then a solution of 4-chloro-2-(methylsulfonyl)pyrimidine (4.47 g, 23.2 mmol) in THF (20 mL) was added dropwise. The reaction mixture was stirred at −72° C. for 2 hrs, then water (50 mL) was added and the mixture was extracted with EtOAc (3×50 mL). The combined organic layers were washed with brine (50 mL), dried over anhydrous Na₂SO₄, filtered and concentrated. The residue was purified by silica gel chromatography (0-79% EtOAc/petroleum ether) to afford the title compound as a light-yellow solid (1.29 g, 22%). MS (ESI⁺): m/z=235.8.

Step B. N-(4-(Tributylstannyl)pyrimidin-2-yl)-5,6-dihydro-4H-pyrrolo[1,2-b]pyrazol-3-amine. The title compound (1.93 g, 67%) was prepared using conditions analogous to those described in Intermediate 57, Step C using N-(4-chloropyrimidin-2-yl)-5,6-dihydro-4H-pyrrolo[1,2-b]pyrazol-3-amine in place of 4-chloro-6-methyl-N-(1-methyl-1H-pyrazol-4-yl)pyrimidin-2-amine. MS (ESI⁺): m/z=492.5.

### Intermediate 68. N-(1-(2,2-Difluoroethyl)-1H-pyrazol-3-yl)-4-(tributylstannyl)pyrimidin-2-amine

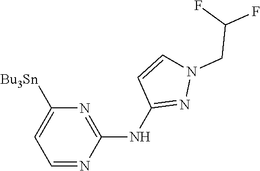

N-(1-(2,2-Difluoroethyl)-1H-pyrazol-3-yl)-4-(tributylstannyl)pyrimidin-2-amine was prepared in a manner analogous to Intermediate 43 using 1-(2,2-difluoroethyl)-1H-pyrazol-3-amine in place of 5-chloro-1-methyl-1H-pyrazol-4-amine. LCMS (ESI): Mass calcd. for C₂₁H₃₅F₂N₅Sn 515.2 m/z, found 514.2.

### Intermediate 69. N-(2-(Methoxymethyl)-2H-1,2,3-triazol-4-yl)-4-(tributylstannyl)pyrimidin-2-amine

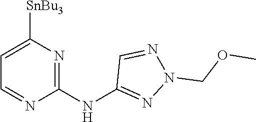

Step A. 4-Chloro-N-(2-(methoxymethyl)-2H-1,2,3-triazol-4-yl)pyrimidin-2-amine. A solution of 2-(methoxymethyl)-2H-1,2,3-triazol-4-amine (70 mg, 0.55 mmol) in THF (3 mL) was evacuated and backfilled with N₂ and then cooled to −72° C. Then, LiHMDS (1 mL, 1 mmol, 1 M in THF) was added and the resulting mixture was stirred at −72° C. for 30 min, followed by the addition of a solution of 4-chloro-2-(methylsulfonyl)pyrimidine (100 mg, 0.52 mmol) in THF (2 mL). The reaction mixture was stirred at −72° C. for 2 hrs then room temperature for 5 hrs. After that time, water (50 mL) was added and the mixture was stirred at room temperature for 10 min. The mixture was filtered and the filter cake was washed with water. The filtrate was concentrated to afford the title compound (70 mg, 55%). MS (ESI⁺): m/z=240.9.

Step B. N-(2-(Methoxymethyl)-2H-1,2,3-triazol-4-yl)-4-(tributylstannyl)pyrimidin-2-amine. A mixture of 4-chloro-N-(2-(methoxymethyl)-2H-1,2,3-triazol-4-yl)pyrimidin-2-amine (90 mg, 0.37 mmol), Pd(PPh₃)₄ (45 mg, 0.039 mmol) and (SnBu₃)₂ (500 mg, 0.862 mmol) in 1,4-dioxane (3 mL) was purged with N₂ three times and then the mixture was heated at 100° C. for 16 hrs. After that time, saturated aqueous KF (10 mL) was added and the mixture was stirred at room temperature for 1 h. The precipitate was removed by filtration and the filtrate was extracted with EtOAc (2×20 mL). The combined organic layers were washed with brine, dried over anhydrous Na₂SO₄, filtered and concentrated. The residue was purified by silica gel chromatography (0-50%) EtOAc/petroleum ether to provide the title compound as a colorless oil (42 mg, 22%). MS (ESI⁺): m/z=497.1.

### Intermediate 70. (*R)-4-Methyl-6,7-dihydro-4H-pyrazolo[5,1-c][1,4]oxazin-3-amine

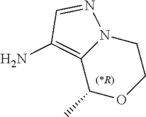

Step A. But-3-yn-2-yl methanesulfonate. To a solution of but-3-yn-2-ol (167 g, 2.38 mol) in anhydrous DCM (1.7 L) at 0° C. were added DIEA (830 mL, 4.77 mol) slowly followed by methanesulfonic anhydride (498 g, 2.86 mol). The resulting mixture was stirred at 0° C. for 10 min, then was allowed to warm to room temperature over 12 hrs. After that time, 10% aqueous NH₄Cl (1 L) was added dropwise followed by extraction with DCM (3×500 mL). The combined organic layers were dried over anhydrous Na₂SO₄, filtered and concentrated. The residue was purified by silica gel chromatography (0-33% EtOAc/petroleum ether) to afford the title compound as a yellow oil (282 g, 80%). ¹H NMR (400 MHz, CDCl₃) δ 5.26-5.12 (m, 1H) 3.06-2.97 (m, 3H) 2.72 (d, J=2.3 Hz, 1H) 1.59-1.52 (m, 3H).

Step B. ((2-(But-3-yn-2-yloxy)ethoxy)methyl)benzene. To a suspension of NaH (71.7 g, 1.79 mol, 60% in mineral oil) in dry 2-MeTHF (2 L) at 0° C. was added 2-(benzyloxy)ethan-1-ol (182 g, 1.20 mol) dropwise, and the resulting mixture was stirred at room temperature for 30 min. Then, the mixture was cooled to 0° C. and but-3-yn-2-yl methanesulfonate (265 g, 1.79 mol) was added. The cooling bath was removed and the mixture was allowed to warm to room temperature and was stirred at room temperature for 11.5 hrs. Then, the reaction was cooled to 0° C. and 10% aqueous NH₄Cl (500 mL) was added dropwise. The mixture was extracted with EtOAc (3×500 mL), the organic layers combined, dried over anhydrous Na₂SO₄, filtered and concentrated. The residue was purified by silica gel chromatography (0-17% EtOAc/petroleum ether) to provide the title compound as a yellow oil (135 g, 55%). MS (ESI⁺): m/z=205.0.

Step C. 5-(1-(2-(Benzyloxy)ethoxy)ethyl)-1H-pyrazole. A mixture of ((2-(but-3-yn-2-yloxy)ethoxy)methyl)benzene (120 g, 587 mol) and TMSCHN₂ (734 mL, 2 M in diethyl ether) was degassed and purged with N₂ three times and then the mixture was heated at 135° C. for 12 hrs. The mixture was concentrated and the residue was purified by silica gel chromatography (0-100% EtOAc/petroleum ether) to afford the title compound as a brown oil (73 g, 50%). MS (ESI⁺): m/z=247.0.

Step D. 2-(1-(1H-Pyrazol-5-yl)ethoxy)ethan-1-ol. To a solution of 5-(1-(2-(benzyloxy)ethoxy)ethyl)-1H-pyrazole (73.0 g, 296 mol) in EtOH (700 mL) under H₂ were added 10% Pd/C (5.00 g, 4.17 mmol) and 10% Pd(OH)₂ (5.00 g, 3.56 mmol). The resulting mixture was heated at 80° C. under H₂ for 12 hrs. The solution was filtered through diatomaceous earth and the filtrate was concentrated. The residue was purified by silica gel chromatography (0-25% MeOH/DCM) to provide the title compound as a colorless oil (30 g, 65%). ¹H NMR (400 MHz, DMSO-d₆) δ 12.64 (br s, 1H), 8.05-7.25 (m, 1H), 6.51-6.01 (m, 1H), 4.87-4.24 (m, 2H), 3.45 (br s, 2H), 3.36-3.21 (m, 2H), 1.48-1.30 (m, 3H).

Step E. 4-Methyl-6,7-dihydro-4H-pyrazolo[5,1-c][1,4]oxazine. To a solution of 2-(1-(1H-pyrazol-5-yl)ethoxy)ethan-1-ol (30.0 g, 192 mmol) in DCM (300 mL) was added 2-(tributyl-λ⁵-phosphaneylidene)acetonitrile (46.4 g, 192 mmol) and the resulting mixture was heated at 50° C. for 12 hrs. The reaction mixture was concentrated and the residue was purified by silica gel chromatography (0-50% EtOAc/petroleum ether) to afford the title compound as a colorless oil (17 g, 64%). MS (ESI⁺): m/z=139.0.

Step F. 4-Methyl-3-nitro-6,7-dihydro-4H-pyrazolo[5,1-c][1,4]oxazine. To a solution of 4-methyl-6,7-dihydro-4H-pyrazolo[5,1-c][1,4]oxazine (16.9 g, 122 mmol) in H₂SO₄ (170 mL) at 0° C. was added a mixture of HNO₃ (90.4 mL, 1.41 mol, 70% purity) in H₂SO₄ (120 mL) and the resulting mixture was stirred at 20° C. for 5 hrs. Then, the mixture was cooled to 0° C., ice-water (500 mL) was added and the mixture was extracted with DCM (2.5 L). The organic layer was washed with saturated aqueous NaHCO₃ (400 mL), dried over anhydrous Na₂SO₄, filtered and concentrated. The residue was purified by silica gel chromatography (0-25% EtOAc/petroleum ether) to afford the title compound as a yellow solid (6 g, 27%). MS (ESI⁺): m/z=183.9.

Step G. (*R)-4-Methyl-6,7-dihydro-4H-pyrazolo[5,1-c][1,4]oxazin-3-amine. To a solution of 4-methyl-3-nitro-6,7-dihydro-4H-pyrazolo[5,1-c][1,4]oxazine (6.80 g, 37.1 mmol) in MeOH (100 mL) under H₂ was added 10% Pd/C (2.00 g, 37.1 mmol). The resulting mixture was heated at 30° C. under H₂ for 12 hrs. The reaction mixture was filtered through diatomaceous earth and the filtrate was concentrated. The residue was purified by silica gel chromatography (0-90% EtOAc/petroleum ether) to afford a mixture of enantiomers. The diastereomers were separated by chiral SFC (DAICEL CHIRALPAK IC, 10 μm, 30×250 mm, isocratic elution: 30% IPA (0.1% of 25% aqueous NH₃), 70% CO₂)) to provide the title compound, the second-eluting isomer, as a brown oil (3.1 g, 90%). MS (ESI⁺): m/z=154.0.

### Intermediate 71. tert-Butyl (2-(3-amino-1H-pyrazol-1-yl)ethyl)carbamate

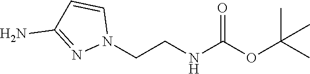 [SMILES: CC(C)(C)OC(=O)NCCn1ccc(N)n1]

Step A. tert-Butyl (2-(3-nitro-1H-pyrazol-1-yl)ethyl)carbamate. A solution of 3-nitro-1H-pyrazole (1.00 g, 8.84 mmol) in DMF (30 mL) was cooled to 0° C., then NaH (707 mg, 17.7 mmol, 60% in mineral oil) and the resulting mixture was allowed to warm to room temperature over 30 min. Then, tert-butyl (2-bromoethyl)carbamate (2.58 g, 11.5 mmol) was added and the mixture was stirred at room temperature for 16 hrs. Water (100 mL) was added and the mixture was extracted with EtOAc (6×30 mL). The combined organic layers were dried over anhydrous Na₂SO₄, filtered and concentrated. The residue was purified by silica gel chromatography (0-30% EtOAc/petroleum ether) to afford the title compound as a colorless oil (2.8 g, 57%). ¹H NMR (400 MHz, DMSO-d₆) δ 8.04 (d, J=2.8 Hz, 1H), 7.97 (d, J=2.4 Hz, 1H), 7.03 (d, J=2.4 Hz, 2H), 6.99-6.96 (m, 1H), 4.25 (t, J=5.6 Hz, 2H), 1.32 (s, 9H).

Step B. tert-Butyl (2-(3-amino-1H-pyrazol-1-yl)ethyl)carbamate. A mixture of tert-butyl (2-(3-nitro-1H-pyrazol-1-yl)ethyl)carbamate (2.80 g, 5.03 mmol) and wet Pd/C (800 mg) in anhydrous EtOH (20 mL) was sparged with H₂ three times and then stirred at room temperature for 6 hrs under an H₂ atmosphere (15 psi). The reaction mixture was filtered through a pad of diatomaceous earth and the filtrate was concentrated to afford a colorless oil. The oil was purified twice by silica gel chromatography (50-100% EtOAc/petroleum ether followed by 0-7% MeOH/DCM) to provide the title compound as a white solid (800 mg, 70%). ¹H NMR (400 MHz, DMSO-d₆) δ 7.23 (d, J=2.0 Hz, 1H), 6.85-6.82 (m, 1H), 5.35 (d, J=2.0 Hz, 1H), 4.53 (s, 2H), 3.83 (t, J=6.8 Hz, 2H), 3.23-3.18 (m, 2H), 1.37 (s, 9H).

(*S)-4-methyl-6,7-dihydro-4H-pyrazolo[5,1-c][1,4]oxazin-3-amine

### Intermediate 72. 1-(3-Fluoro-1-methylpiperidin-4-yl)-1H-pyrazol-4-amine

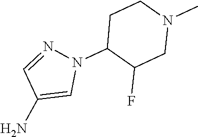

Step A. tert-Butyl 3-fluoro-4-((methylsulfonyl)oxy)piperidine-1-carboxylate. A mixture of tert-butyl 3-fluoro-4-hydroxypiperidine-1-carboxylate (10.0 g, 45.6 mmol) and TEA (12.7 mL, 91.2 mmol) in anhydrous DCM (150 mL) was cooled to 0° C. and then methanesulfonic anhydride (12.0 g, 68.4 mmol) was added and the resulting mixture was warmed to room temperature over 6 hrs. The mixture was concentrated and purified by silica gel chromatography (0-35% EtOAc/petroleum ether) to afford the title compound as a white solid (8.2 g, 60%). ¹H NMR (400 MHz, CDCl₃) δ 5.01-4.87 (m, 1H), 4.80-4.64 (m, 1H), 3.97-3.32 (m, 4H), 3.18-3.02 (m, 3H), 2.24-1.82 (m, 2H), 1.47 (s, 9H).

Step B. tert-Butyl 3-fluoro-4-(4-nitro-1H-pyrazol-1-yl)piperidine-1-carboxylate. A mixture of tert-butyl 3-fluoro-4-((methylsulfonyl)oxy)piperidine-1-carboxylate (8.2 g, 27.6 mmol), 4-nitro-1H-pyrazole (3.74 g, 33.1 mmol) and K₂CO₃ (7.62 g, 55.2 mmol) in anhydrous DMF (100 mL) was heated to 90° C. for 16 hrs. The mixture was cooled to room temperature, diluted with water (60 mL) and extracted with EtOAc (3×50 mL). The combined organic layers were washed with brine (40 mL), dried over anhydrous Na₂SO₄, filtered and concentrated. The residue was purified by silica gel chromatography (0-30% EtOAc/petroleum ether) to afford the title compound as a white jelly (5.9 g, 64%). ¹H NMR (400 MHz, DMSO-d₆) δ 9.08 (s, 1H), 8.34 (s, 1H), 4.10-3.93 (m, 1H), 3.39-2.87 (m, 5H), 2.11-1.90 (m, 2H), 1.46-1.37 (m, 9H).

Step C. 3-Fluoro-4-(4-nitro-1H-pyrazol-1-yl)piperidine hydrochloride. A mixture of tert-butyl 3-fluoro-4-(4-nitro-1H-pyrazol-1-yl)piperidine-1-carboxylate (5.9 g, 18.8 mol) and 4 M HCl in 1,4-dioxane (40 mL) in anhydrous 1,4-dioxane (40 mL) was stirred at room temperature for 2 hrs, then concentrated to afford the title compound, which was used without further purification (4.2 g). MS (ESI⁺): m/z=215.1.

Step D. 3-Fluoro-1-methyl-4-(4-nitro-1H-pyrazol-1-yl)piperidine. A mixture of 3-fluoro-4-(4-nitro-1H-pyrazol-1-yl)piperidine hydrochloride (4.2 g, 16.1 mmol), formaldehyde (19.1 mL, 255 mmol, 37%) and formic acid (20 mL) was heated at 100° C. for 16 hrs. Then, saturated aqueous NaHCO₃ (200 mL) was added and the mixture was extracted with EtOAc (3×50 mL). The combined organic layers were dried over anhydrous Na₂SO₄, filtered and concentrated. The residue was purified by silica gel chromatography (0-100% EtOAc/petroleum ether) to afford the title compound as a white solid (2.74 g, 71%). MS (ESI⁺): m/z=229.1.

Step E. 1-(3-Fluoro-1-methylpiperidin-4-yl)-1H-pyrazol-4-amine. To a solution of 3-fluoro-1-methyl-4-(4-nitro-1H-pyrazol-1-yl)piperidine (1.2 g, 5.26 mmol) in EtOH (10 mL) was added 10% Pd/C (300 mg) and the resulting mixture was stirred at room temperature for 16 hrs under a H₂ atmosphere (15 psi). The mixture was filtered and the filtrate was concentrated to afford the title compound as a pale yellow solid (940 mg), which was used without further purification. MS (ESI⁺): m/z=199.1.

### Intermediate 73. 1-(1-(Oxetan-3-yl)pyrrolidin-3-yl)-1H-pyrazol-4-amine

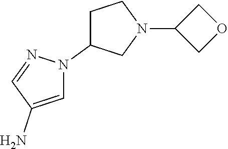 [SMILES: Nc1cnn(C2CCN(C3COC3)C2)c1]

Step A. tert-Butyl 3-(4-nitro-1H-pyrazol-1-yl)pyrrolidine-1-carboxylate. To a mixture of 4-nitro-1H-pyrazole (2.00 g, 17.7 mmol), tert-butyl 3-hydroxypyrrolidine-1-carboxylate (3.97 g, 21.2 mmol) and PPh₃ (5.57 g, 21.2 mmol) in anhydrous THF (100 mL) was added DBAD (5.30 g, 23.0 mmol) in three portions, and the resulting mixture was stirred at room temperature for 16 hrs. The mixture was poured into water (200 mL) and extracted with EtOAc (3×80 mL). The combined organic layers were washed with brine (2×20 mL), dried over anhydrous Na₂SO₄, filtered and concentrated. The residue was purified by silica gel chromatography (0-30% EtOAc/petroleum ether) to afford the title compound as a light-yellow oil (4.17 g, 75%). ¹H NMR (400 MHz, CDCl₃) δ 8.18 (s, 1H), 8.10 (s, 1H), 4.93-4.88 (m, 1H), 3.92-3.70 (m, 2H), 3.65-3.53 (m, 2H), 2.48-2.37 (m, 2H), 1.48 (s, 9H).

Step B. 4-Nitro-1-(pyrrolidin-3-yl)-1H-pyrazole. A mixture of tert-butyl 3-(4-nitro-1H-pyrazol-1-yl)pyrrolidine-1-carboxylate (2.47 g, 8.75 mmol) and 4 M HCl in 1,4-dioxane (30 mL) in anhydrous 1,4-dioxane (20 mL) was stirred at room temperature for 2 hrs. The reaction mixture was concentrated to provide the title compound as a white solid (1.93 g, 95%). ¹H NMR (400 MHz, DMSO-d₆) δ 9.88-9.69 (m, 1H), 9.60-9.43 (m, 1H), 9.09-9.05 (m, 1H), 8.39-8.34 (m, 1H), 5.25 (tt, J=4.0, 7.2 Hz, 1H), 3.71-3.49 (m, 3H), 2.47-2.30 (m, 3H).

Step C. 4-Nitro-1-(1-(oxetan-3-yl)pyrrolidin-3-yl)-1H-pyrazole. A mixture of 4-nitro-1-(pyrrolidin-3-yl)-1H-pyrazole (2.43 g, 11.1 mmol), oxetan-3-one (3.25 mL, 55.6 mmol), TEA (3.55 mL, 25.6 mmol) and AcOH (3.84 mL, 67.2 mmol) in DCE (40 mL) was stirred at room temperature for 3 hrs and then NaBH₃CN (3.49 g, 55.6 mmol) was added and the resulting mixture was heated at 60° C. for 12 hrs. The reaction mixture was cooled to room temperature, poured in water (40 mL) and extracted with DCM (3×40 mL). The combined organic layers were dried over anhydrous Na₂SO₄, filtered and concentrated. The residue was purified by silica gel chromatography (10-100% EtOAc/petroleum ether) to provide the title compound as a colorless oil (2.62 g, 89%). MS (ESI⁺): m/z=239.2.

Step D. 1-(1-(Oxetan-3-yl)pyrrolidin-3-yl)-1H-pyrazol-4-amine. To a mixture of 4-nitro-1-(1-(oxetan-3-yl)pyrrolidin-3-yl)-1H-pyrazole (2.62 g, 9.94 mmol) and NH₄Cl (2.13 g, 39.7 mmol) in EtOH (40 mL) and water (8 mL) was added Fe powder (2.78 g, 49.7 mmol) and the resulting mixture was heated at 70° C. for 16 hrs. The mixture was filtered through a pad of diatomaceous earth and the filtrate was concentrated. The residue was purified by silica gel chromatography (0-10% MeOH/DCM) to afford the title compound as a purple oil (1.3 g, 57%). MS (ESI⁺): m/z=209.1.

### Intermediate 74. (R)-3-Hydroxy-3-(3-(4-methoxy-6-(2-(methylsulfonyl)pyrimidin-4-yl)pyridin-2-yl)isoxazol-5-yl)-1-methylpyrrolidin-2-one

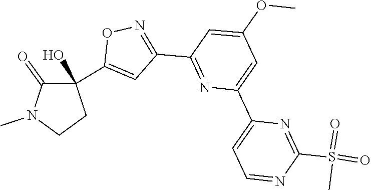 [SMILES: COc1cc(-c2cc([C@]3(O)CCN(C)C3=O)on2)nc(-c2ccnc(S(C)(=O)=O)n2)c1]

Step A. 2-Bromo-4-methoxy-6-vinylpyridine. A flask containing 2,6-dibromo-4-methoxypyridine (5.00 g, 18.7 mmol), potassium trifluoro(vinyl)borate (2.50 g, 18.7 mmol) and K₃PO₄ (12.0 g, 56.5 mmol) in 1,4-dioxane/water (5/1, 90 mL) was evacuated and refilled with Ar three times. Then, Pd(dppf)Cl₂·DCM (2.00 g, 2.73 mmol) was added and the resulting mixture was heated at 90° C. for 16 hrs. After that time, the mixture was cooled to room temperature, poured into water (90 mL) and extracted with EtOAc (3×100 mL). The combined organic layers were washed with brine (100 mL), dried over anhydrous Na₂SO₄, filtered and concentrated. The residue was purified by silica gel chromatography (0-50% EtOAc/petroleum ether) to afford the title compound as a white solid (3.3 g, 52%). MS (ESI⁺): m/z=214.8.

Step B. 6-Bromo-4-methoxypicolinaldehyde. A mixture of K₂OsO₄·2H₂O (533 mg, 1.45 mmol) and 2-bromo-4-methoxy-6-vinylpyridine (3.30 g, 15.4 mmol) in THF (48 mL) and water (12 mL) was cooled to 0° C. and the mixture was stirred at 0° C. for 10 min. Then, NaIO₄ (9.85 g, 46.1 mmol) was added and the resulting mixture was stirred at room temperature for 16 hrs. Saturated aqueous Na₂S₂O₃ (50 mL) was added and the mixture was extracted with EtOAc (3×80 mL). The combined organic layers were washed with brine (80 mL), dried over anhydrous Na₂SO₄, filtered and concentrated. The residue was purified by silica gel chromatography (0-50% DCM/petroleum ether) to afford the title compound as a white solid (1.2 g, 35%). MS (ESI⁺): m/z=216.8.

Step C. (E)-6-Bromo-4-methoxypicolinaldehyde oxime. A mixture of 6-bromo-4-methoxypicolinaldehyde (1.20 g, 5.56 mmol), NH₂OH·HCl (480 mg, 6.91 mmol) and NaOAc (900 mg, 11.0 mmol) in EtOH (30 mL) was heated at 45° C. for 3 hrs. After that time, the mixture was poured into water (50 mL) and extracted with EtOAc (3×50 mL). The combined organic layers were washed with brine (50 mL), dried over anhydrous Na₂SO₄, filtered and concentrated. The residue was purified by silica gel chromatography (0-40% EtOAc/petroleum ether) to afford the title compound as a white solid (995 mg, 72%). MS (ESI⁺): m/z=232.6.

Step D. (Z)-6-Bromo-N-hydroxy-4-methoxypicolinimidoyl chloride. A mixture of (E)-6-bromo-4-methoxypicolinaldehyde oxime (1.1 g, 4.8 mmol) and NCS (726 mg, 5.38 mmol) in DMF (10 mL) was stirred at room temperature for 3 hrs. Saturated aqueous Na₂S₂O₃ (20 mL) was added and the mixture was extracted with EtOAc (3×30 mL). The combined organic layers were dried over anhydrous Na₂SO₄, filtered and concentrated. The residue was purified by silica gel chromatography (0-50% EtOAc/petroleum ether) to afford the title compound as a yellow oil (1.7 g, in DMF). MS (ESI⁺): m/z=266.9.

Step E. (R)-3-(3-(6-Bromo-4-methoxypyridin-2-yl)isoxazol-5-yl)-3-hydroxy-1-methylpyrrolidin-2-one. A mixture of (R)-3-ethynyl-3-hydroxy-1-methylpyrrolidin-2-one (700 mg, 5.03 mmol), (Z)-6-bromo-N-hydroxy-4-methoxypicolinimidoyl chloride (1.7 g, 6.4 mmol) and NaHCO₃ (1.11 g, 13.2 mmol) in EtOAc (20 mL) was stirred at room temperature for 4 hrs. The mixture was then concentrated and the residue purified by reverse-phase HPLC (Xtimate C18 column, 10 μm, 40×150 mm; 25-55% ACN/H₂O (with 0.05% NH₃ in water and 10 mM NH₄HCO₃)) to afford the title compound as a white solid (907 mg, 48%). MS (ESI⁺): m/z=370.1.

Step F. (R)-3-Hydroxy-3-(3-(4-methoxy-6-(2-(methylthio)pyrimidin-4-yl)pyridin-2-yl)isoxazol-5-yl)-1-methylpyrrolidin-2-one. A flask containing (R)-3-(3-(6-bromo-4-methoxypyridin-2-yl)isoxazol-5-yl)-3-hydroxy-1-methylpyrrolidin-2-one (907 mg, 2.46 mmol), 2-(methylthio)-4-(tributylstannyl)pyrimidine (1.1 g, 2.6 mmol) and TEA (705 μL, 5.06 mmol) in toluene (15 mL) was evacuated and refilled with Ar three times, then Pd(PPh₃)₄ (288 mg, 0.249 mmol) was added. The resulting mixture was heated at 120° C. for 16 hrs then gradually cooled to room temperature. Saturated aqueous KF (60 mL) was added over 2 min, and then the mixture was stirred at room temperature for 2 hrs. The mixture was extracted with EtOAc (3×70 mL), and the combined organic layers were washed with brine (90 mL), dried over anhydrous Na₂SO₄, filtered and concentrated. The residue was purified by reverse-phase HPLC (Xtimate C18 column, 10 μm, 40×150 mm; 30-60% ACN/H₂O (with 0.05% NH₃ in water and 10 mM NH₄HCO₃)) to afford the title compound as a white solid (135 mg, 11%). MS (ESI⁺): m/z=435.9 [M+23]⁺.

Step G. (R)-3-Hydroxy-3-(3-(4-methoxy-6-(2-(methylsulfonyl)pyrimidin-4-yl)pyridin-2-yl)isoxazol-5-yl)-1-methylpyrrolidin-2-one. A mixture of (R)-3-hydroxy-3-(3-(4-methoxy-6-(2-(methylthio)pyrimidin-4-yl)pyridin-2-yl)isoxazol-5-yl)-1-methylpyrrolidin-2-one (110 mg, 0.266 mmol) and potassium peroxymonosulfate (330 g, 0.537 mmol) in THF (1 mL), water (1 mL) and acetone (1 mL) was stirred at room temperature for 16 hrs. The mixture was then poured into water (15 mL) and extracted with DCM (3×20 mL). The combined organic layers were dried over anhydrous Na₂SO₄, filtered and concentrated to afford the title compound as a white solid (110 mg, 79%). MS (ESI⁺): m/z=446.1.

### Intermediate 75. (R)-3-Hydroxy-1-methyl-3-(1-(6-(2-(methylsulfonyl)pyrimidin-4-yl)pyridin-2-yl)-1H-1,2,3-triazol-4-yl)pyrrolidin-2-one

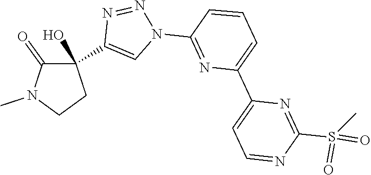 [SMILES: CN1CC[C@@](O)(c2cn(-c3cccc(-c4ccnc(S(C)(=O)=O)n4)n3)nn2)C1=O]

Step A. 4-(6-Bromopyridin-2-yl)-2-(methylthio)pyrimidine. To a solution of 2-(methylthio)-4-(tributylstannyl)pyrimidine (6.50 g, 15.7 mmol) and 2,6-dibromopyridine (7.42 g, 31.3 mmol) in toluene (80 mL) was added Pd(PPh₃)₄ (1.81 g, 1.57 mmol). The mixture was sparged with Ar and then heated at 120° C. for 16 hrs. The reaction mixture was cooled to room temperature, 2 M aqueous KF (60 mL) was added and the mixture was extracted with DCM (80 mL). The organic layer was washed with brine (60 mL), dried over anhydrous Na₂SO₄, filtered and concentrated. The residue was purified by silica gel chromatography (0-8% EtOAc/petroleum ether) to afford the title compound as a white solid (4.4 g, 48%). MS (ESI⁺): m/z=283.7.

Step B. (R)-3-Hydroxy-1-methyl-3-(1-(6-(2-(methylthio)pyrimidin-4-yl)pyridin-2-yl)-1H-1,2,3-triazol-4-yl)pyrrolidin-2-one. A mixture of (R)-3-ethynyl-3-hydroxy-1-methylpyrrolidin-2-one (666 mg, 4.79 mmol), 4-(6-bromopyridin-2-yl)-2-(methylthio)pyrimidine (1.35 g, 4.79 mmol), (1S,2S)—N₁,N₂-dimethylcyclohexane-1,2-diamine (136 mg, 0.956 mmol) and CuI (182 mg, 0.956 mmol) in DMSO (15 mL) was sparged with Ar, then NaN₃ (440 mg, 6.77 mmol) was added. Then, a solution of sodium ascorbate (190 mg, 0.959 mmol) in water (3 mL) was added slowly and the resulting mixture was heated at 70° C. for 12 hrs. The mixture was cooled to room temperature and the pH of the mixture adjusted to pH 9-10 by the addition of saturated aqueous Na₂CO₃. The mixture was extracted with EtOAc (2×30 mL), the combined organic layers were washed with brine (30 mL), dried over anhydrous Na₂SO₄, filtered and concentrated. The residue was purified by silica gel chromatography (0-100% EtOAc/petroleum ether) to afford the title compound as a white solid (900 mg, 44%). ¹H NMR (400 MHz, CDCl₃) δ 8.84 (s, 1H), 8.63 (d, J=5.2 Hz, 1H), 8.46 (d, J=7.6 Hz, 1H), 8.17 (d, J=8.4 Hz, 1H), 8.03-7.97 (m, 1H), 7.85 (d, J=5.2 Hz, 1H), 4.40 (s, 1H), 3.82-3.73 (m, 1H), 3.57-3.47 (m, 1H), 3.07-3.01 (m, 1H), 3.00 (s, 3H), 2.66 (s, 3H), 2.56-2.48 (m, 1H).

Step C. (R)-3-Hydroxy-1-methyl-3-(1-(6-(2-(methylsulfonyl)pyrimidin-4-yl)pyridin-2-yl)-1H-1,2,3-triazol-4-yl)pyrrolidin-2-one. A mixture of potassium peroxymonosulfate (3.21 g, 5.22 mmol) and (R)-3-hydroxy-1-methyl-3-(1-(6-(2-(methylthio)pyrimidin-4-yl)pyridin-2-yl)-1H-1,2,3-triazol-4-yl)pyrrolidin-2-one (1.00 g, 2.61 mmol) in THF (10 mL), H₂O (10 mL) and acetone (10 mL) was stirred at room temperature for 16 hrs. The reaction mixture was poured into water (20 mL) and extracted with DCM (3×20 mL). The combined organic layers were dried over anhydrous Na₂SO₄, filtered and concentrated. The residue was purified by silica gel chromatography (0-10% MeOH/EtOAc) to afford the title compound as a white solid (500 mg, 42%). ¹H NMR (400 MHz, CDCl₃) δ 9.07 (d, J=5.2 Hz, 1H), 8.82 (s, 1H), 8.66-8.61 (m, 1H), 8.54 (d, J=5.2 Hz, 1H), 8.33-8.26 (m, 1H), 8.16-8.09 (m, 1H), 3.82-3.73 (m, 1H), 3.60-3.51 (m, 1H), 3.47 (s, 3H), 3.11-3.03 (m, 1H), 3.01 (s, 3H), 2.60-2.47 (m, 1H).

### Intermediate 76. N-(1-Methyl-1H-pyrazol-4-yl)-4-(tributylstannyl)pyrimidin-2-amine

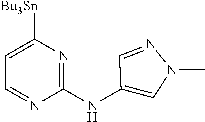

Step A. 4-Chloro-N-(1-methyl-1H-pyrazol-4-yl)pyrimidin-2-amine. A flask containing 1-methyl-1H-pyrazol-4-amine (8.10 g, 83.4 mmol) in THF (150 mL) was evacuated and backfilled with N₂ three times. The solution was cooled to −72° C. and LDA (52 mL, 104 mmol, 2 M in THF/heptane/ethylbenzene) was added dropwise. The resulting solution was stirred at −72° C. for 30 min. Then, 4-chloro-2-(methylsulfonyl)pyrimidine (8.00 g, 41.5 mmol) was suspended in THF (50 mL) and the solution was added to the reaction mixture via a syringe. The resulting mixture was stirred at −72° C. for 2 hrs then warmed to 0° C. Water (30 mL) was added to the mixture dropwise, then the mixture was filtered through a pad of diatomaceous earth. The filter cake was washed with EtOAc (300 mL), and the filtrate was washed with brine (3×50 mL), dried over anhydrous Na₂SO₄, filtered and concentrated. The residue was purified by silica gel chromatography (50-100 EtOAc/petroleum ether) to afford the title compound as a yellow solid (2.95 g, 34%). MS (ESI⁺): m/z=209.7.

Step B. N-(1-Methyl-1H-pyrazol-4-yl)-4-(tributylstannyl)pyrimidin-2-amine. To a solution of 4-chloro-N-(1-methyl-1H-pyrazol-4-yl)pyrimidin-2-amine (800 mg, 3.82 mmol) and LiCl (970 mg, 22.9 mmol) in 1,4-dioxane (10 mL) was added hexa-n-butylditin (4.76 g, 8.21 mmol) and the mixture was sparged with N₂. Then, Pd₂(dba)3 (175 mg, 0.191 mmol) and tricyclohexylphosphine (107 mg, 0.382 mmol) were added and the resulting mixture heated at 110° C. for 16 hrs. The mixture was diluted with EtOAc (100 mL) and filtered. The filter cake was washed with EtOAc (100 mL) and the filtrate was washed with brine (3×30 mL), dried over anhydrous Na₂SO₄, filtered and concentrated. The residue was purified by silica gel chromatography (20-60% EtOAc/petroleum ether) to afford the title compound as a yellow oil (720 mg, 40%). ¹H NMR (400 MHz, DMSO-d₆) δ 9.26 (s, 1H), 8.24-8.09 (m, 1H), 7.83 (s, 1H), 7.52 (s, 1H), 6.93-6.70 (m, 1H), 3.78 (s, 3H), 1.69-1.41 (m, 6H), 1.38-1.24 (m, 6H), 1.21-0.99 (m, 6H), 0.93-0.75 (m, 9H).

### Intermediate 77. (R)-3-(1-(6-Chloro-4-(difluoromethyl)pyridin-2-yl)-1H-1,2,3-triazol-4-yl)-3-hydroxy-1-methylpyrrolidin-2-one

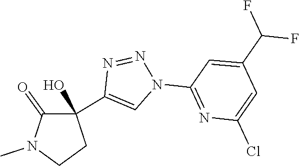 [SMILES: CN1CC[C@@](O)(c2cn(-c3cc(C(F)F)cc(Cl)n3)nn2)C1=O]

Step A. 2,6-Dichloro-4-(difluoromethyl)pyridine. To a solution of 2,6-dichloropyridine-4-carboxaldehyde (2.00 g, 11.4 mmol) in DCM (100 mL) under N₂ at −78° C. was added DAST (5.50 g, 31.1 mmol). The resulting mixture was warmed to room temperature and then stirred at room temperature for 1 h. The reaction mixture was poured into saturated aqueous NaHCO₃ (300 mL) and extracted with DCM (2×100 mL). The combined organic layers were washed with brine (200 mL), dried over anhydrous Na₂SO₄, filtered and concentrated. The residue was purified by silica gel chromatography (0-13% EtOAc/petroleum ether) to afford the title compound as a colorless oil (1.9 g, 84%). MS (ESI⁺): m/z=198.0.

Step B. (R)-3-(1-(6-Chloro-4-(difluoromethyl)pyridin-2-yl)-1H-1,2,3-triazol-4-yl)-3-hydroxy-1-methylpyrrolidin-2-one. A mixture of 2,6-dichloro-4-(difluoromethyl)pyridine (1.43 g, 7.22 mmol), (R)-3-ethynyl-3-hydroxy-1-methylpyrrolidin-2-one (500 mg, 3.59 mmol), (1S,2S)-N¹,-N²-dimethylcyclohexane-1,2-diamine (103 mg, 0.724 mmol) and CuI (138 mg, 0.725 mmol) in DMSO (12 mL) was sparged with Ar, then sodium azide (230 mg, 3.54 mmol) was added. Then, a solution of sodium ascorbate (145 mg, 0.732 mmol) in water (2 mL) was added slowly to the reaction mixture and the resulting mixture was heated at 70° C. for 16 hrs. The reaction was cooled to room temperature and the mixture was diluted with EtOAc (50 mL) and filtered through a pad of diatomaceous earth. The filtrate was washed with sequentially with brine (3×50 mL) and water (50 mL), dried over anhydrous Na₂SO₄, filtered and concentrated. The residue was purified by silica gel chromatography (50-100% EtOAc/petroleum ether) to afford the title compound as a white-yellow solid (190 mg, 15%). MS (ESI⁺): m/z=344.1.

### Intermediate 78. (R)-3-(1-(6-Chloro-4-(methoxymethyl)pyridin-2-yl)-1H-1,2,3-triazol-4-yl)-3-hydroxy-1-methylpyrrolidin-2-one

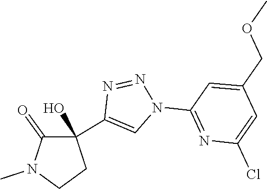

Step A. 2,6-Dichloro-4-(methoxymethyl)pyridine. To a solution of (2,6-dichloropyridin-4-yl)methanol (2.50 g, 14.0 mmol) in anhydrous DMF (48 mL) were added cesium carbonate (7.32 g, 22.5 mmol) and iodomethane (2.80 g, 19.7 mmol) dropwise. The resulting mixture was heated at 45° C. for 12 hrs. Water (50 mL) was added and the mixture was extracted with EtOAc (2×50 mL). The combined organic layers were washed with saturated aqueous LiCl (2×100 mL), dried over anhydrous Na₂SO₄, filtered and concentrated. The residue was purified by silica gel chromatography (0-20% EtOAc/petroleum ether) to afford the title compound as a colorless oil (2.0 g, 55%). MS (ESI⁺): m/z=191.8.

Step B. (R)-3-(1-(6-Chloro-4-(methoxymethyl)pyridin-2-yl)-1H-1,2,3-triazol-4-yl)-3-hydroxy-1-methylpyrrolidin-2-one. The title compound (300 mg, 16%) was prepared using conditions analogous to those described in Intermediate 77 Step B using 2,6-dichloro-4-(methoxymethyl)pyridine in place of 2,6-dichloro-4-(difluoromethyl)pyridine. MS (ESI⁺): m/z=338.1.

### Intermediate 79. (R)-3-(1-(6-Bromo-4-methoxypyridin-2-yl)-1H-1,2,3-triazol-4-yl)-3-hydroxy-1-methylpyrrolidin-2-one

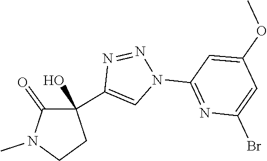 [SMILES: COc1cc(Br)nc(-n2cc([C@]3(O)CCN(C)C3=O)nn2)c1]

The title compound (700 mg, 49%) was prepared using conditions analogous to those described in Intermediate 77 Step B using 2,6-dibromo-4-methoxypyridine in place of 2,6-dichloro-4-(difluoromethyl)pyridine. MS (ESI⁺): m/z=370.0.

### Example 1: (3R,5S)-3-Hydroxy-1-methyl-3-(3-(6-(2-((1-methyl-1H-pyrazol-3-yl)amino)pyrimidin-4-yl)pyridin-2-yl)isoxazol-5-yl)-5-(trifluoromethyl)pyrrolidin-2-one

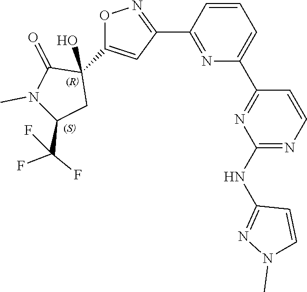

To a vial was added (3R,5S)-3-(3-(6-bromopyridin-2-yl)isoxazol-5-yl)-3-hydroxy-1-methyl-5-(trifluoromethyl)pyrrolidin-2-one (Intermediate 8, 97 mg, 0.24 mmol), N-(1-methyl-1H-pyrazol-3-yl)-4-(trimethylstannyl)pyrimidin-2-amine (Intermediate 10, 105 mg, 0.31 mmol) and 1,4-dioxane (1.2 mL). To this mixture was added tetrakis(triphenylphosphine)-palladium(0) (41 mg, 0.04 mmol) and the resulting dark brown mixture was heated at 110° C. for 12 hours. After the reaction mixture cooled to room temperature, it was filtered through a syringe filter and purified by RP-HPLC via acidic conditions using column C7 to afford the title compound (as its TFA salt) as a light-yellow solid (10 mg, 7%). ¹H NMR (500 MHz, Methanol-d₄) δ 8.70 (d, J=5.7 Hz, 1H), 8.60 (dd, J=7.9, 1.0 Hz, 1H), 8.31-8.23 (m, 1H), 8.19-8.10 (m, 2H), 7.63 (d, J=2.4 Hz, 1H), 7.27 (s, 1H), 6.47 (s, 1H), 4.58-4.45 (m, 1H), 3.92 (s, 3H), 3.12 (dd, J=14.6, 8.5 Hz, 1H), 3.07 (s, 3H), 2.46 (dd, J=14.6, 4.9 Hz, 1H). LC-MS (ESI): Mass calcd. for C₂₂H₁₉F₃N₈O₃ 500.1 m/z found 501.2 [M+H]⁺. The TFA salt could be converted to the corresponding free base by passage through a carbonate cartridge.

### Example 2: (R)-3-Hydroxy-1-methyl-3-(3-(6-(2-((1-methyl-1H-pyrazol-3-yl)amino)pyrimidin-4-yl)-4-(trifluoromethyl)pyridin-2-yl)isoxazol-5-yl)pyrrolidin-2-one

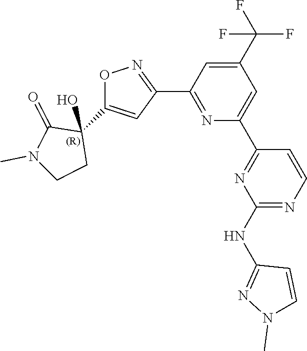

The title compound (11 mg, TFA salt, 10%) was prepared using conditions analogous to those described in Example 1 using (R)-3-(3-(6-chloro-4-(trifluoromethyl)pyridin-2-yl)isoxazol-5-yl)-3-hydroxy-1-methylpyrrolidin-2-one (Intermediate 6, 66 mg, 0.18 mmol) in place of 1-methyl-5-(trifluoromethyl)pyrrolidin-2-one. ¹H NMR (400 MHz, Methanol-d₄) δ 8.87-8.78 (m, 1H), 8.73 (d, J=5.6 Hz, 1H), 8.51-8.40 (m, 1H), 8.10 (d, J=5.6 Hz, 1H), 7.63 (d, J=2.4 Hz, 1H), 7.29 (s, 1H), 6.53 (d, J=2.4 Hz, 1H), 3.92 (s, 3H), 3.62 (ddt, J=10.4, 7.0, 3.2 Hz, 2H), 3.00 (s, 3H), 2.80 (ddd, J=13.5, 7.1, 5.0 Hz, 1H), 2.46 (ddd, J=14.0, 8.0, 6.4 Hz, 1H). LC-MS (ESI): Mass calcd. for C₂₂H₁₉F₃N₈O₃ 500.2 m/z, found 501.2 [M+H]⁺. The TFA salt could be converted to the corresponding free base by passage through a carbonate cartridge.

### Example 3: (R)-4,4-Difluoro-3-hydroxy-1-methyl-3-(3-(6-(2-((1-methyl-1H-pyrazol-4-yl)amino)pyrimidin-4-yl)pyridin-2-yl)isoxazol-5-yl)pyrrolidin-2-one

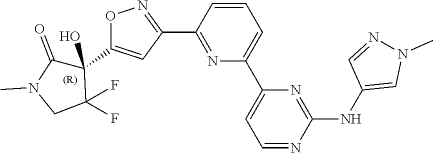

To a vial was added (R)-4,4-difluoro-3-hydroxy-1-methyl-3-(3-(6-(2-(methylsulfonyl)pyrimidin-4-yl)pyridin-2-yl)isoxazol-5-yl)pyrrolidin-2-one (Intermediate 7, 80 mg, 0.18 mmol), 1-methyl-1H-pyrazol-4-amine (34 mg, 0.35 mmol), DMSO (anhydrous, 0.5 mL), and TFA (0.03 mL, 0.44 mmol). The resulting reaction mixture was heated at 150° C. for 90 min. After the reaction mixture cooled to room temperature, it was filtered through a syringe filter and purified by RP-HPLC via acidic conditions using column C7 to afford the title compound as yellow solid (5 mg, TFA salt, 5%). ¹H NMR (400 MHz, Methanol-d₄) δ 8.59-8.50 (m, 2H), 8.20 (dd, J=7.9, 1.1 Hz, 1H), 8.12 (t, J=7.8 Hz, 1H), 8.02 (s, 1H), 7.89 (d, J=5.3 Hz, 1H), 7.67 (s, 1H), 7.30 (s, 1H), 4.06-3.95 (m, 2H), 3.93 (s, 3H), 3.06 (s, 3H). LC-MS (ESI): Mass calcd. for C₂₁H₁₈F₂N₈O₃ 468.1 m/z, found 469.1 [M+H]⁺. The TFA salt could be converted to the corresponding free base by passage through a carbonate cartridge.

### Example 4: (R)-3-Hydroxy-1-methyl-3-(3-(6-(2-((1-methyl-1H-pyrazol-4-yl)amino)pyrimidin-4-yl)-4-(trifluoromethyl)pyridin-2-yl)isoxazol-5-yl)pyrrolidin-2-one

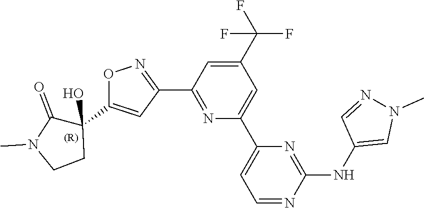

To a vial was added (R)-3-Hydroxy-1-methyl-3-(3-(6-(2-(methylsulfonyl)pyrimidin-4-yl)-4-(trifluoromethyl)pyridin-2-yl)isoxazol-5-yl)pyrrolidin-2-one (Intermediate 6, 80 mg, 0.17 mmol), 1-methyl-1H-pyrazol-4-amine (34 mg, 0.35 mmol), DMSO (anhydrous, 0.5 mL), and TFA (0.03 mL, 0.41 mmol). The reaction mixture was heated at 150° C. for 90 min and allowed to cool to room temperature. After the reaction mixture cooled to room temperature, it was filtered through a syringe filter. The compound was subjected to purification via RP-HPLC under acidic conditions using column C7. Yielded a yellow solid (44 mg, TFA salt, 43%). ¹H NMR (400 MHz, Methanol-d₄) δ 8.77 (s, 1H), 8.62 (d, J=5.1 Hz, 1H), 8.39 (d, J=1.4 Hz, 1H), 7.99 (s, 1H), 7.88 (d, J=5.1 Hz, 1H), 7.66 (s, 1H), 7.25 (s, 1H), 3.91 (s, 3H), 3.63-3.56 (m, 2H), 2.98 (s, 3H), 2.84-2.73 (m, 1H), 2.51-2.39 (m, 1H). LC-MS (ESI): Mass calcd. for C₂₂H₁₉F₃N₈O₃ 500.2 m/z, found 501.1 [M+H]⁺. The TFA salt could be converted to the corresponding free base by passage through a carbonate cartridge.

### Example 5: (R)-3-Hydroxy-1-methyl-3-(3-(6-(2-((1-methyl-1H-pyrazol-4-yl)amino)pyrimidin-4-yl)pyridin-2-yl)isoxazol-5-yl)pyrrolidin-2-one-4,4,5,5-d₄

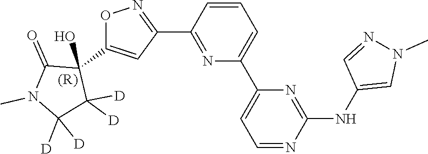

(R)-3-Hydroxy-1-methyl-3-(3-(6-(2-(methylsulfonyl)pyrimidin-4-yl)pyridin-2-yl)isoxazol-5-yl)pyrrolidin-2-one-4,4,5,5-d₄ (Intermediate 9, 100 mg, 0.24 mmol), 1-methyl-1H-pyrazol-4-amine (46 mg, 0.48 mmol), DMSO (anhydrous, 0.8 mL), and TFA (0.05 mL, 0.60 mmol) were combined. The resulting solution was heated at 150° C. for 90 min. The solution was then filtered through a syringe filter and directly subjected to RP-HPLC via basic conditions using column C8 to provide the title compound as a yellow solid (7 mg, 7%). ¹H NMR (600 MHz, Methanol-d₄) δ 8.55 (d, J=5.0 Hz, 1H), 8.51 (dd, J=7.8, 1.1 Hz, 1H), 8.14 (dd, J=7.8, 1.1 Hz, 1H), 8.08 (t, J=7.8 Hz, 1H), 8.00 (s, 1H), 7.81 (d, J=5.1 Hz, 1H), 7.64 (s, 1H), 7.18 (s, 1H), 3.91 (s, 3H), 2.97 (s, 3H). LC-MS (ESI): Mass calcd. for C₂₁H₁₆D₄N₈O₃ 436.2 m/z, found 437.2 [M+H]⁺. The TFA salt could be converted to the corresponding free base by passage through a carbonate cartridge.

### Example 6: (3R,5S)-3-Hydroxy-1-methyl-3-(3-(6-(2-((1-methyl-1H-pyrazol-4-yl)amino)pyrimidin-4-yl)pyridin-2-yl)isoxazol-5-yl)-5-(trifluoromethyl)pyrrolidin-2-one

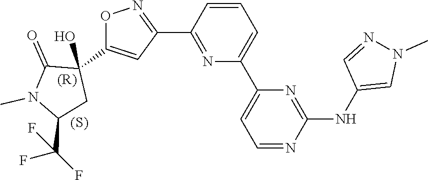

(3R,5S)-3-Hydroxy-1-methyl-3-(3-(6-(2-(methylsulfonyl)pyrimidin-4-yl)pyridin-2-yl)isoxazol-5-yl)-5-(trifluoromethyl)pyrrolidin-2-one (Intermediate 8, 100 mg, 0.21 mmol), 1-methyl-1H-pyrazol-4-amine (40 mg, 0.41 mmol), DMSO (anhydrous, 0.8 mL), and TFA (0.04 mL, 0.52 mmol) were combined. The resulting solution was heated at 150° C. for 90 min. The solution was then filtered through a syringe filter and directly subjected to RP-HPLC via basic conditions using column C8 to provide the title compound as a yellow solid (6 mg, 6%). LC-MS (ESI): Mass calcd. for C₂₂H₁₉F₃N₈O₃ 500.1 m/z found 501.2 [M+H]⁺.

### Example 7: (R)-3-(3-(6-(2-((1-Ethyl-1H-pyrazol-4-yl)amino)pyrimidin-4-yl)pyridin-2-yl)isoxazol-5-yl)-3-hydroxy-1-methylpyrrolidin-2-one-4,4,5,5-d₄

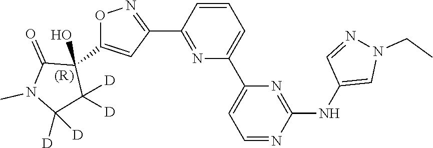

(R)-3-Hydroxy-1-methyl-3-(3-(6-(2-(methylsulfonyl)pyrimidin-4-yl)pyridin-2-yl)isoxazol-5-yl)pyrrolidin-2-one-4,4,5,5-d₄ (Intermediate 9, 50 mg, 0.12 mmol), 1-ethyl-1H-pyrazol-4-amine (40 mg, 0.36 mmol), DMSO (anhydrous, 0.4 mL), and TFA (0.03 mL, 0.36 mmol) were combined. The resulting solution was heated at 150° C. for 90 min. The solution was then filtered through a syringe filter and directly subjected to RP-HPLC via basic conditions using column C8 to provide the title compound a yellow solid (9 mg, 17%). ¹H NMR (400 MHz, Methanol-d₄) δ 8.59-8.49 (m, 2H), 8.18-8.06 (m, 2H), 8.04 (d, J=0.8 Hz, 1H), 7.82 (d, J=5.1 Hz, 1H), 7.67 (s, 1H), 7.19 (s, 1H), 4.20 (q, J=7.3 Hz, 3H), 2.97 (s, 3H), 1.49 (t, J=7.3 Hz, 3H). LC-MS (ESI): Mass calcd. for C₂₂H₁₈D₄N₈O₃ 450.2 m/z, found 451.0 [M+H]⁺.

### Example 8: (R)-3-Hydroxy-1-methyl-3-(3-(6-(2-((1-methyl-1H-pyrazol-3-yl)amino)pyrimidin-4-yl)pyridin-2-yl)isoxazol-5-yl)pyrrolidin-2-one-4,4,5,5-d₄

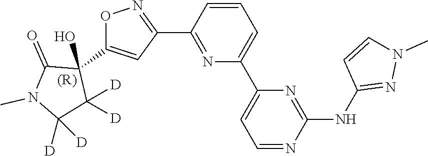

(R)-3-Hydroxy-1-methyl-3-(3-(6-(2-(methylsulfonyl)pyrimidin-4-yl)pyridin-2-yl)isoxazol-5-yl)pyrrolidin-2-one-4,4,5,5-d₄ (Intermediate 9, 150 mg, 0.36 mmol), 1-methyl-1H-pyrazol-3-amine (104 mg, 1.05 mmol), DMSO (anhydrous, 1.2 mL), and TFA (0.08 mL, 1.07 mmol) were combined. The resulting solution was heated at 150° C. for 90 min. The solution was then filtered through a syringe filter and directly subjected to RP-HPLC via acidic conditions using column C7 to provide a TFA salt of the title compound as a yellow solid (14 mg, TFA salt, 7%). ¹H NMR (500 MHz, Methanol-d₄) δ 8.70 (d, J=5.8 Hz, 1H), 8.59 (dd, J=7.8, 1.1 Hz, 1H), 8.24 (dd, J=7.8, 1.1 Hz, 1H), 8.16 (d, J=7.8 Hz, 1H), 8.15-8.11 (m, 1H), 7.63 (d, J=2.4 Hz, 1H), 7.21 (s, 1H), 6.46 (s, 1H), 3.92 (s, 3H), 2.97 (s, 3H). LC-MS (ESI): Mass calcd. for C₂₁H₁₆D₄N₈O₃ 436.2 m/z, found 436.7 [M+H]⁺. The TFA salt could be converted to the corresponding free base by passage through a carbonate cartridge.

### Example 9: (R)-3-(3-(6-(2-((1-(1,3-Dihydroxypropan-2-yl)-3-methoxy-1H-pyrazol-4-yl)amino)pyrimidin-4-yl)pyridin-2-yl)isoxazol-5-yl)-3-hydroxy-1-methylpyrrolidin-2-one

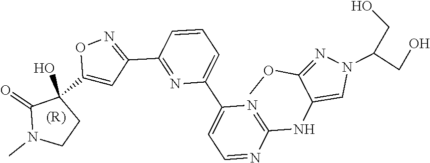

(R)-3-Hydroxy-1-methyl-3-(3-(6-(2-(methylsulfonyl)pyrimidin-4-yl)pyridin-2-yl)isoxazol-5-yl)pyrrolidin-2-one (Intermediate 13, 150 mg, 0.36 mmol), 3-methoxy-1-(oxetan-3-yl)-1H-pyrazol-4-amine (Intermediate 20, 61 mg, 0.36 mmol), DMSO (anhydrous, 0.4 mL), and TFA (0.03 mL, 0.36 mmol) were combined. The resulting solution was heated at 150° C. for 90 min. The reaction mixture was then filtered through a syringe filter and directly purified by RP-HPLC via basic conditions with column C8 to provide the title compound as a yellow solid (20 mg, 32%). ¹H NMR (400 MHz, Methanol-d₄) δ 8.53 (dd, J=7.8, 1.2 Hz, 1H), 8.49 (d, J=5.1 Hz, 1H), 8.14 (dd, J=7.8, 1.2 Hz, 1H), 8.06 (t, J=7.8 Hz, 1H), 7.93 (s, 1H), 7.81 (d, J=5.1 Hz, 1H), 7.18 (s, 1H), 4.15 (q, J=6.2 Hz, 1H), 3.99-3.86 (m, 7H), 3.62-3.56 (m, 2H), 2.97 (s, 3H), 2.76 (ddd, J=13.5, 6.9, 5.0 Hz, 1H), 2.47-2.38 (m, 1H). LC-MS (ESI): Mass calcd. for C₂₄H₂₆N₈O₆ 522.2 m/z, found 523.0 [M+H]⁺.

### Example 10: (R)-4,4-Difluoro-3-hydroxy-1-methyl-3-(3-(6-(2-((1-(1-methylpiperidin-4-yl)-1H-pyrazol-4-yl)amino)pyrimidin-4-yl)pyridin-2-yl)isoxazol-5-yl)pyrrolidin-2-one

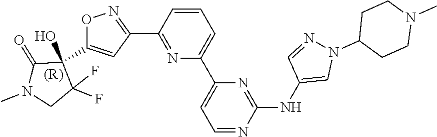

The title compound (3 mg, 5%) was prepared with analogous conditions as described in Example 3 using 1-(1-methylpiperidin-4-yl)-1H-pyrazol-4-amine (60 mg, 0.33 mmol) in place of 1-methyl-1H-pyrazol-4-amine. ¹H NMR (500 MHz, Methanol-d₄) δ 8.56 (d, J=5.1 Hz, 1H), 8.53 (dd, J=7.7, 1.1 Hz, 1H), 8.17 (dd, J=7.8, 1.1 Hz, 1H), 8.14-8.03 (m, 2H), 7.82 (d, J=5.1 Hz, 1H), 7.68 (s, 1H), 7.30 (s, 1H), 4.23-4.16 (m, 1H), 4.06-3.92 (m, 2H), 3.09-2.95 (m, 6H), 2.37-2.16 (m, 12H). LC-MS (ESI): Mass calcd. for C₂₆H₂₇F₂N₉O₃ 551.2 m/z found 551.7 [M+H]⁺. The TFA salt could be converted to the corresponding free base by passage through a carbonate cartridge.

### Example 11: (R)-3-Hydroxy-1-methyl-3-(3-(3-(2-(((S)-tetrahydrofuran-3-yl)amino)pyrimidin-4-yl)phenyl)isoxazol-5-yl)pyrrolidin-2-one

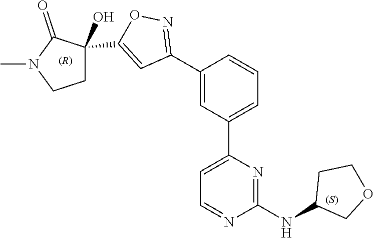

Step A: (R)-3-Hydroxy-1-methyl-3-(3-(3-(2-(methylthio)pyrimidin-4-yl)phenyl)isoxazol-5-yl)pyrrolidin-2-one. A mixture of 4-chloro-2-(methylthio)pyrimidine (620 mg, 3.9 mmol), (R)-3-hydroxy-1-methyl-3-(3-(3-(4,4,5,5-tetramethyl-1,3,2-dioxaborolan-2-yl)phenyl)isoxazol-5-yl)pyrrolidin-2-one (Intermediate 11, 1.53 g, 3.98 mmol), bis(triphenylphosphine)palladium(ii) chloride (582 mg, 0.814 mmol) and K₂CO₃ (3 mL, 6 mmol, 2M in H₂O) in 1,4-dioxane/EtOH (5 mL/1.0 mL) was degassed for 10 min with nitrogen. This mixture was heated at 170° C. for 10 min and then allowed to cool to room temperature. The mixture was partitioned between EtOAc/H₂O (2 mL/2 mL) and extracted with EtOAc (3×2 mL). The organic extracts were combined and concentrated. The residue was purified by silica gel chromatography (10% MeOH in DCM) to provide the title compound (1.1 g, 75%). MS (ESI): Mass calcd. for C₁₉H₁₈N₄O₃S, 382.1; m/z found, 383.3 [M+H]⁺.

Step B: (R)-3-Hydroxy-1-methyl-3-(3-(3-(2-(methylsulfonyl)pyrimidin-4-yl)phenyl)isoxazol-5-yl)pyrrolidin-2-one. To (R)-3-hydroxy-1-methyl-3-(3-(3-(2-(methylthio)pyrimidin-4-yl)phenyl)isoxazol-5-yl)pyrrolidin-2-one (1.1 g, 2.9 mmol) in DCM (15 mL), was added m-CPBA (545 mg, 3.16 mmol). This mixture was heated at 50° C. for 2 h. After the reaction mixture cooled to room temperature, aqueous saturated NaHCO₃ (10 mL) was added. The organic extract was separated and concentrated to provide the title compound (910 mg, 76%). MS (ESI): Mass calcd. for C₁₉H₁₈N₄O₅S, 414.1; m/z found, 415.3 [M+H]⁺.

Step C: (R)-3-Hydroxy-1-methyl-3-(3-(3-(2-(((S)-tetrahydrofuran-3-yl)amino)pyrimidin-4-yl)phenyl)isoxazol-5-yl)pyrrolidin-2-one. A mixture of (S)-3-aminotetrahydrofuran (29 mg, 0.34 mmol), (R)-3-hydroxy-1-methyl-3-(3-(3-(2-(methylsulfonyl)pyrimidin-4-yl)phenyl)isoxazol-5-yl)pyrrolidin-2-one (140 mg, 0.34 mmol) and Et₃N (0.24 mL, 1.7 mmol) in DMSO (1 mL) was heated at 120° C. for 2 h and allowed to cool to rt. After the reaction mixture was concentrated, the residue was purified with HPLC (Xtimate C18 column, 10 μm, 150×40 mm; 20-50% ACN/H₂O with 0.05% NH₃·H₂O) to provide the title compound (62 mg, 44%). MS (ESI): Mass calcd. for C₂₂H₂₃N₅O₄, 421.2; m/z found, 442.3 [M+H]⁺. ¹H NMR (500 MHz, Chloroform-d) δ 10.32 (br s, 1H), 8.53 (s, 1H), 8.20 (d, J=8.5 Hz, 2H), 8.05-7.97 (m, 1H), 7.68 (t, J=7.8 Hz, 1H), 7.27-7.22 (m, 1H), 6.79 (s, 1H), 4.83-4.73 (m, 1H), 4.26-4.16 (m, 1H), 4.16-4.04 (m, 1H), 4.02-3.93 (m, 1H), 3.88 (dd, J=9.4, 4.2 Hz, 1H), 3.77-3.66 (m, 1H), 3.57-3.47 (m, 1H), 3.04 (s, 3H), 2.89-2.80 (m, 1H), 2.56-2.49 (m, 1H), 2.46-2.38 (m, 1H), 2.23-2.19 (m, 1H).

### Example 12: (R)-3-Hydroxy-1-methyl-3-(3-(3-(2-(((R)-tetrahydrofuran-3-yl)amino)pyrimidin-4-yl)phenyl)isoxazol-5-yl)pyrrolidin-2-one

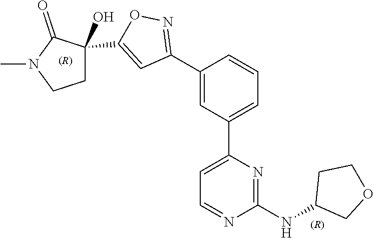

(R)-3-Hydroxy-1-methyl-3-(3-(3-(2-(((R)-tetrahydrofuran-3-yl)amino)pyrimidin-4-yl)phenyl)isoxazol-5-yl)pyrrolidin-2-one (41 mg, 28%) was prepared in a manner analogous to Example 11 Step C using (R)-3-aminotetrahydrofuran in place of (S)-3-aminotetrahydrofuran. MS (ESI): Mass calcd. for C₂₂H₂₃N₅O₄, 421.2; m/z found, 422.2 [M+H]⁺. ¹H NMR (500 MHz, Chloroform-d) δ 10.35 (br s, 1H), 8.50 (s, 1H), 8.18 (d, J=9.6 Hz, 2H), 8.00 (dd, J=7.8, 1.5 Hz, 1H), 7.66 (t, J=7.8 Hz, 1H), 7.24 (d, J=6.2 Hz, 1H), 6.79 (d, J=1.0 Hz, 1H), 4.86-4.70 (m, 1H), 4.25-4.16 (m, 1H), 4.11 (q, J=7.9 Hz, 1H), 4.04-3.95 (m, 1H), 3.87 (dd, J=9.4, 4.2 Hz, 1H), 3.75-3.68 (m, 1H), 3.60-3.50 (m, 1H), 3.04 (s, 3H), 2.88-2.76 (m, 1H), 2.56-2.48 (m, 1H), 2.48-2.39 (m, 1H), 2.27-2.19 (m, 1H).

### Example 13: (R)-3-Hydroxy-3-(3-(3-(2-(((1 S,3S)-3-hydroxycyclopentyl)amino)pyrimidin-4-yl)phenyl)isoxazol-5-yl)-1-methylpyrrolidin-2-one

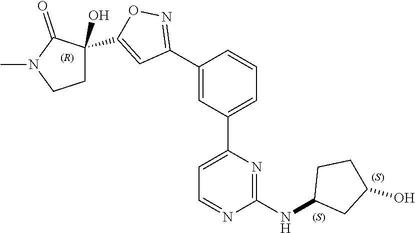

(R)-3-Hydroxy-3-(3-(3-(2-(((1 S,3S)-3-hydroxycyclopentyl)amino)pyrimidin-4-yl)phenyl)isoxazol-5-yl)-1-methylpyrrolidin-2-one (48 mg, 48%) was prepared in a manner analogous to Example 11 Step C using (1 S,3S)-3-aminocyclopentanol in place of (S)-3-aminotetrahydrofuran. MS (ESI): Mass calcd. for C₂₃H₂₅N₅O₄, 435.2; m/z found, 436.2 [M+H]⁺. ¹H NMR (500 MHz, Methanol-d₄) δ 8.55 (t, J=1.8 Hz, 1H), 8.30 (d, J=5.3 Hz, 1H), 8.19 (d, J=7.8 Hz, 1H), 7.98-7.88 (m, 1H), 7.59 (t, J=7.8 Hz, 1H), 7.13 (d, J=5.3 Hz, 1H), 6.95 (s, 1H), 4.65-4.54 (m, 1H), 4.46-4.37 (m, 1H), 3.64-3.51 (m, 2H), 2.97 (s, 3H), 2.81-2.67 (m, 1H), 2.45-2.37 (m, 1H), 2.37-2.27 (m, 1H), 2.18-2.05 (m, 2H), 1.89-1.78 (m, 1H), 1.72-1.53 (m, 2H).

### Example 14: (R)-3-Hydroxy-3-(3-(3-(2-(((1 S,3R)-3-hydroxycyclopentyl)amino)pyrimidin-4-yl)phenyl)isoxazol-5-yl)-1-methylpyrrolidin-2-one

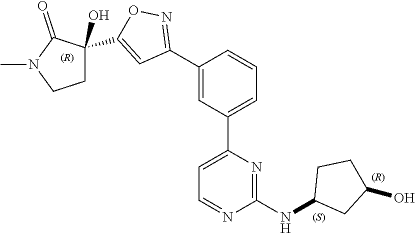

(R)-3-Hydroxy-3-(3-(3-(2-(((1 S,3R)-3-hydroxycyclopentyl)amino)pyrimidin-4-yl)phenyl)isoxazol-5-yl)-1-methylpyrrolidin-2-one (50 mg, 50%) was prepared in a manner analogous to Example 11 Step C using (1 S,3R)-3-aminocyclopentanol in place of (S)-3-aminotetrahydrofuran. MS (ESI): Mass calcd. for C₂₃H₂₅N₅O₄, 435.2; m/z found, 436.3 [M+H]⁺. ¹H NMR (500 MHz, Methanol-d₄) δ 8.54 (t, J=1.8 Hz, 1H), 8.30 (d, J=5.3 Hz, 1H), 8.22-8.11 (m, 1H), 7.94 (ddd, J=7.7, 1.8, 1.1 Hz, 1H), 7.59 (t, J=7.8 Hz 1H), 7.13 (d, J=5.3 Hz, 1H), 6.95 (s, 1H), 4.42 (s, 1H), 4.39-4.29 (m, 1H), 3.65-3.51 (m, 2H), 2.96 (s, 3H), 2.80-2.69 (m, 1H), 2.48-2.30 (m, 2H), 2.20-2.06 (m, 1H), 1.94-1.76 (m, 3H), 1.68-1.56 (m, 1H).

### Example 15: (R)-3-(3-(3-(2-((6,7-Dihydro-5H-pyrazolo[5,1-b][1,3]oxazin-3-yl)amino)pyrimidin-4-yl)phenyl)isoxazol-5-yl)-3-hydroxy-1-methylpyrrolidin-2-one

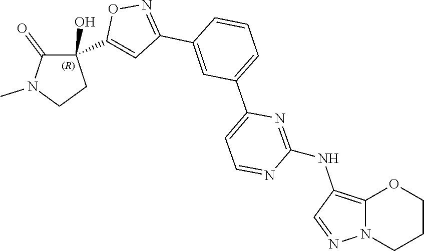

Step A: (R)-3-(3-(3-(2-Chloropyrimidin-4-yl)phenyl)isoxazol-5-yl)-3-hydroxy-1-methylpyrrolidin-2-one. 2,4-Dichloropyrimidine. (447 mg, 3.00 mmol), (R)-3-hydroxy-1-methyl-3-(3-(3-(4,4,5,5-tetramethyl-1,3,2-dioxaborolan-2-yl)phenyl)isoxazol-5-yl)pyrrolidin-2-one (Intermediate 11, 1.15 g, 3.00 mmol), bis(triphenylphosphine)palladium(ii) chloride (582 mg, 0.814 mmol) and K₂CO₃ (3 mL, 6 mmol, 2 M in H₂O) in 1,4-dioxane/EtOH (7.0 mL/1.0 mL) was degassed for 10 min with nitrogen. Then the mixture was heated at 170° C. for 10 min and then allowed to cool to room temperature. The mixture was partitioned between with EtOAc/H₂O (2 mL/2 mL) and extracted with EtOAc (3×2 mL). The organic extracts were combined and concentrated. The residue was purified by silica gel chromatography (10% MeOH in DCM) to provide the title compound (1.01 g, 90.8%). MS (ESI): Mass calcd. for C₁₈H₁₅ClN₄O₃, 370.1; m/z found, 370.9 [M+H]⁺.

Step B: (R)-3-(3-(3-(2-((6,7-Dihydro-5H-pyrazolo[5,1-b][1,3]oxazin-3-yl)amino)pyrimidin-4-yl)phenyl)isoxazol-5-yl)-3-hydroxy-1-methylpyrrolidin-2-one. A mixture of (R)-3-(3-(3-(2-chloropyrimidin-4-yl)phenyl)isoxazol-5-yl)-3-hydroxy-1-methylpyrrolidin-2-one (185 mg, 0.500 mmol), 6,7-dihydro-5h-pyrazolo[5,1-b][1,3]oxazin-3-amine (139 mg, 0.500 mmol), 4,5-bis(diphenylphosphino)-9,9-dimethylxanthene (14 mg, 0.050 mmol), Pd₂(dba)₃ (23 mg, 0.050 mmol) and Cs₂CO₃ (211 mg, 0.650 mmol) in THF (10 mL) was heated at 120° C. for 16 h, and then allowed to cool to room temperature. The mixture was filtered and the filtrate was concentrated. The residue was initially purified with FCC (MeOH/DCM) and then subsequently purified with reverse HPLC (Xtimate C18 column, 10 μm, 150×40 mm; 20-50% ACN/H₂O with 0.05% NH₃·H₂O) to provide the title compound (18 mg, 7.6%). MS (ESI): Mass calcd. for C₂₄H₂₃N₇O₄, 473.2; m/z found, 473.9 [M+H]⁺. ¹H NMR (600 MHz, Methanol-d₄) δ 8.62 (s, 1H), 8.37 (d, J=5.3 Hz, 1H), 8.21 (d, J=7.8 Hz, 1H), 7.99 (d, J=7.9 Hz, 1H), 7.64 (t, J=7.7 Hz, 1H), 7.53 (s, 1H), 7.30 (d, J=5.3 Hz, 1H), 6.96 (s, 1H), 4.44-4.34 (m, 2H), 4.20 (t, J=6.1 Hz, 2H), 3.68-3.55 (m, 2H), 2.99 (s, 3H), 2.81-2.70 (m, 1H), 2.43 (ddd, J=13.6, 8.1, 6.6 Hz, 1H), 2.33 (p, J=6.0 Hz, 2H).

### Example 16: (R)-3-Hydroxy-3-(3-(3-(2-((4-methoxy-1-methyl-1H-pyrazol-3-yl)amino)pyrimidin-4-yl)phenyl)isoxazol-5-yl)-1-methylpyrrolidin-2-one

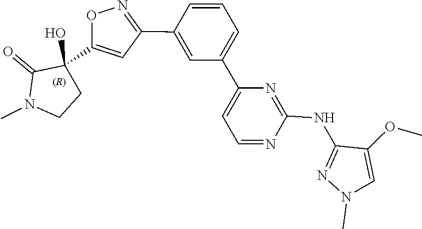

(R)-3-Hydroxy-3-(3-(3-(2-((4-methoxy-1-methyl-1H-pyrazol-3-yl)amino)pyrimidin-4-yl)phenyl)isoxazol-5-yl)-1-methylpyrrolidin-2-one (15 mg, 6.4%) was prepared in a manner analogous to Example 15 Step B using 4-methoxy-1-methyl-1H-pyrazol-3-amine in place of 6,7-dihydro-5h-pyrazolo[5,1-b][1,3]oxazin-3-amine. MS (ESI): Mass calcd. for C₂₃H₂₃N₇O₄, 461.2; m/z found, 461.9 [M+H]⁺. ¹H NMR (600 MHz, Methanol-d₄) δ 8.73 (t, J=1.8 Hz, 1H), 8.49 (d, J=6.0 Hz, 1H), 8.38-8.30 (m, 1H), 8.12-8.06 (m, 1H), 7.71 (t, J=7.8 Hz, 1H), 7.62 (d, J=6.1 Hz, 1H), 7.52 (s, 1H), 7.00 (s, 1H), 3.88 (s, 3H), 3.82 (s, 3H), 3.67-3.55 (m, 2H), 3.00 (s, 3H), 2.81-2.71 (m, 1H), 2.49-2.39 (m, 1H).

### Example 17: (R)-3-Hydroxy-3-(3-(3-(2-((3-methoxy-1-methyl-1H-pyrazol-4-yl)amino)pyrimidin-4-yl)phenyl)isoxazol-5-yl)-1-methylpyrrolidin-2-one

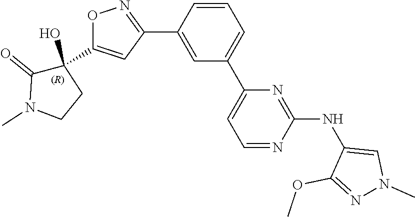

(R)-3-Hydroxy-3-(3-(3-(2-((3-methoxy-1-methyl-1H-pyrazol-4-yl)amino)pyrimidin-4-yl)phenyl)isoxazol-5-yl)-1-methylpyrrolidin-2-one (24 mg, 14%) was prepared in a manner analogous to Example 15 Step B using 3-methoxy-1-methyl-1H-pyrazol-4-amine in place of 6,7-dihydro-5h-pyrazolo[5,1-b][1,3]oxazin-3-amine. MS (ESI): Mass calcd. for C₂₃H₂₃N₇O₄, 461.2; m/z found, 461.9 [M+H]⁺. ¹H NMR (600 MHz, Methanol-d₄) δ 8.64 (t, J=1.8 Hz, 1H), 8.42 (d, J=5.2 Hz, 1H), 8.20 (dt, J=7.8, 1.3 Hz, 1H), 8.05-7.94 (m, 1H), 7.85 (s, 1H), 7.64 (t, J=7.8 Hz, 1H), 7.29 (d, J=5.2 Hz, 1H), 6.98 (s, 1H), 3.95 (s, 3H), 3.79 (s, 3H), 3.65-3.53 (m, 2H), 2.99 (s, 3H), 2.83-2.71 (m, 1H), 2.49-2.37 (m, 1H).

### Example 18: (R)-3-Hydroxy-1-methyl-3-(3-(3-(2-(pyrazolo[1,5-a]pyrimidin-3-ylamino)pyrimidin-4-yl)phenyl)isoxazol-5-yl)pyrrolidin-2-one

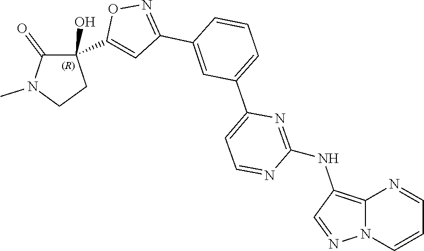

(R)-3-Hydroxy-1-methyl-3-(3-(3-(2-(pyrazolo[1,5-a]pyrimidin-3-ylamino)pyrimidin-4-yl)phenyl)isoxazol-5-yl)pyrrolidin-2-one (14 mg, 7.8%) was prepared in a manner analogous to Example 15 Step B using pyrazolo[1,5-a]pyrimidin-3-amine in place of 6,7-dihydro-5h-pyrazolo[5,1-b][1,3]oxazin-3-amine. MS (ESI): Mass calcd. for C₂₄H₂₀N₈O₃, 468.2; m/z found, 468.9 [M+H]⁺. ¹H NMR (600 MHz, Methanol-d₄) δ 9.00-8.88 (m, 1H), 8.71-8.51 (m, 3H), 8.42 (d, J=5.7 Hz, 1H), 8.29 (s, 1H), 8.05 (d, J=7.8 Hz, 1H), 7.68 (t, J=7.7 Hz, 1H), 7.54 (d, J=5.9 Hz, 1H), 7.08 (dd, J=7.1, 4.0 Hz, 1H), 6.96 (s, 1H), 3.68-3.55 (m, 2H), 3.00 (s, 3H), 2.83-2.73 (m, 1H), 2.49-2.39 (m, 1H).

### Example 19: (R)-3-(3-(3-(2-(3-Amino-6,7-dihydropyrazolo[1,5-a]pyrimidin-4(5H)-)pyrimidin-4-yl)phenyl)isoxazol-5-yl)-3-hydroxy-1-methylpyrrolidin-2-one

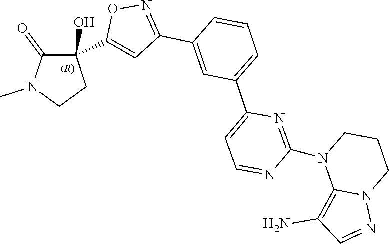

(R)-3-(3-(3-(2-(3-amino-6,7-dihydropyrazolo[1,5-a]pyrimidin-4(5H)-yl)pyrimidin-4-yl)phenyl)isoxazol-5-yl)-3-hydroxy-1-methylpyrrolidin-2-one (4.0 mg, 1.6%) was prepared in a manner analogous to Example 15 Step B using 4,5,6,7-tetrahydropyrazolo[1,5-a]pyrimidin-3-amine in place of 6,7-dihydro-5h-pyrazolo[5,1-b][1,3]oxazin-3-amine. MS (ESI): Mass calcd. for C₂₄H₂₄N₈O₃, 472.2; m/z found, 472.9 [M+H]⁺. ¹H NMR (500 MHz, Methanol-d₄) δ 8.71-8.51 (m, 2H), 8.28-8.16 (m, 1H), 8.06-7.99 (m, 1H), 7.67 (t, J=7.8 Hz, 1H), 7.53-7.43 (m, 1H), 7.30 (s, 1H), 6.98 (s, 1H), 4.45-4.32 (m, 2H), 4.23 (t, J=6.3 Hz, 2H), 3.66-3.56 (m, 2H), 2.99 (s, 3H), 2.84-2.72 (m, 1H), 2.52-2.37 (m, 1H), 2.30-2.20 (m, 2H).

### Example 20: (R)-3-Hydroxy-1-methyl-3-(3-(3-(2-((4,5,6,7-tetrahydropyrazolo[1,5-a]pyrimidin-3-yl)amino)pyrimidin-4-yl)phenyl)isoxazol-5-yl)pyrrolidin-2-one

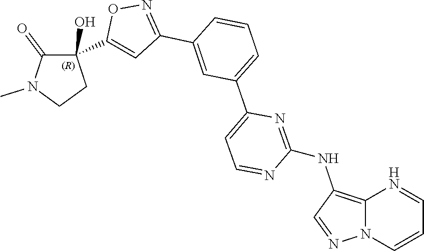

(R)-3-Hydroxy-1-methyl-3-(3-(3-(2-((4,5,6,7-tetrahydropyrazolo[1,5-a]pyrimidin-3-yl)amino)pyrimidin-4-yl)phenyl)isoxazol-5-yl)pyrrolidin-2-one (48 mg, 20%) was prepared in a manner analogous to Example 15 Step B using 4,5,6,7-tetrahydropyrazolo[1,5-a]pyrimidin-3-amine in place of 6,7-dihydro-5h-pyrazolo[5,1-b][1,3]oxazin-3-amine. MS (ESI): Mass calcd. for C₂₄H₂₄N₈O₃, 472.2; m/z found, 472.9 [M+H]⁺. ¹H NMR (500 MHz, Methanol-d₄) δ 8.75-8.69 (m, 1H), 8.68 (d, J=1.8 Hz, 1H), 8.33 (d, J=8.1 Hz, 1H), 8.12-8.03 (m, 1H), 7.72 (t, J=7.8 Hz, 1H), 7.67 (d, J=5.3 Hz, 1H), 7.62 (d, J=3.1 Hz, 1H), 4.58-4.47 (m, 2H), 4.34 (t, J=6.2 Hz, 2H), 3.69-3.56 (m, 2H), 3.00 (s, 3H), 2.83-2.71 (m, 1H), 2.49-2.41 (m, 1H), 2.41-2.32 (m, 2H).

### Example 21: (R)-3-(3-(3-(2-((5-Amino-1-methyl-1H-pyrazol-4-yl)amino)pyrimidin-4-yl)phenyl)isoxazol-5-yl)-3-hydroxy-1-methylpyrrolidin-2-one

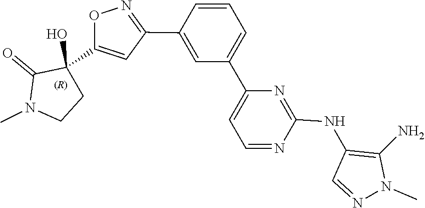

(R)-3-(3-(3-(2-((5-Amino-1-methyl-1H-pyrazol-4-yl)amino)pyrimidin-4-yl)phenyl)isoxazol-5-yl)-3-hydroxy-1-methylpyrrolidin-2-one (10 mg, 3.9%) was prepared in a manner analogous to Example 15 Step B using 1-methyl-1H-pyrazole-4,5-diamine in place of 6,7-dihydro-5h-pyrazolo[5,1-b][1,3]oxazin-3-amine. MS (ESI): Mass calcd. for C₂₂H₂₂N₈O₃, 446.2; m/z found, 446.9 [M+H]⁺. ¹H NMR (600 MHz, Methanol-d₄) δ 8.73 (t, J=1.8 Hz, 1H), 8.49 (d, J=6.0 Hz, 1H), 8.37-8.28 (m, 1H), 8.14-8.05 (m, 1H), 7.71 (t, J=7.8 Hz, 1H), 7.62 (d, J=6.1 Hz, 1H), 7.52 (s, 1H), 7.00 (s, 1H), 3.88 (s, 3H), 3.82 (s, 3H), 3.69-3.54 (m, 2H), 3.00 (s, 3H), 2.82-2.75 (m, 1H), 2.51-2.38 (m, 1H).

### Example 22: (R)-3-(3-(3-(2-((6,7-Dihydro-4H-pyrazolo[5,1-c][1,4]oxazin-2-yl)amino)pyrimidin-4-yl)phenyl)isoxazol-5-yl)-3-hydroxy-1-methylpyrrolidin-2-one

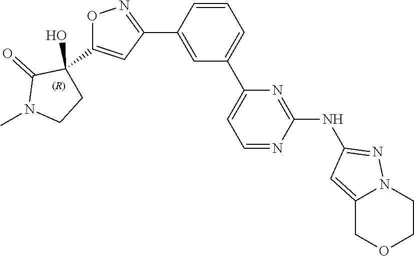

(R)-3-(3-(3-(2-((6,7-Dihydro-4H-pyrazolo[5,1-c][1,4]oxazin-2-yl)amino)pyrimidin-4-yl)phenyl)isoxazol-5-yl)-3-hydroxy-1-methylpyrrolidin-2-one (22 mg, 4.6%) was prepared in a manner analogous to Example 15 Step B using 6,7-dihydro-4h-pyrazolo[5,1-c][1,4]oxazin-2-amine in place of 6,7-dihydro-5h-pyrazolo[5,1-b][1,3]oxazin-3-amine. MS (ESI): Mass calcd. for C₂₄H₂₃N₇O₄, 473.2; m/z found, 474.4 [M+H]⁺. ¹H NMR (500 MHz, DMSO-d₆) δ 9.94 (s, 1H), 8.65 (t, J=1.8 Hz, 1H), 8.56 (d, J=5.2 Hz, 1H), 8.34-8.24 (m, 1H), 8.12-8.01 (m, 1H), 7.70 (t, J=7.8 Hz, 1H), 7.53 (d, J=5.2 Hz, 1H), 7.11 (s, 1H), 6.56 (s, 1H), 4.09 (t, J=5.3 Hz, 2H), 4.02 (t, J=5.3 Hz, 1H), 3.56-3.38 (m, 2H), 2.86 (s, 3H), 2.65-2.58 (m, 1H), 2.36-2.23 (m, 1H).

### Example 23: (R)-2-((4-(3-(5-(3-Hydroxy-1-methyl-2-oxopyrrolidin-3-yl)isoxazol-3-yl)phenyl)pyrimidin-2-yl)amino)-5-methyl-6,7-dihydropyrazolo[1,5-a]pyrazin-4(5H)-one

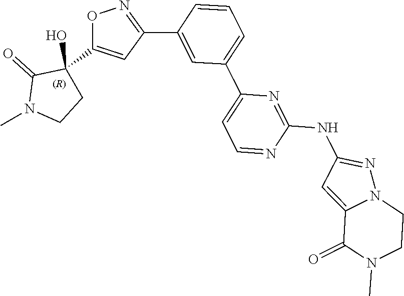

(R)-2-((4-(3-(5-(3-Hydroxy-1-methyl-2-oxopyrrolidin-3-yl)isoxazol-3-yl)phenyl)pyrimidin-2-yl)amino)-5-methyl-6,7-dihydropyrazolo[1,5-a]pyrazin-4(5H)-one (18 mg, 3.6%) was prepared in a manner analogous to Example 15 Step B using 2-amino-5-methyl-6,7-dihydropyrazolo[1,5-a]pyrazin-4(5H)-one in place of 6,7-dihydro-5h-pyrazolo[5,1-b][1,3]oxazin-3-amine. MS (ESI): Mass calcd. for C₂₅H₂₄N₈O₄, 500.2; m/z found, 500.9 [M+H]⁺. ¹H NMR (500 MHz, Methanol-d₄) δ 8.70 (t, J=1.6 Hz, 1H), 8.56 (d, J=5.3 Hz, 1H), 8.33-8.25 (m, 1H), 8.12-7.99 (m, 1H), 7.68 (t, J=7.7 Hz, 1H), 7.46 (d, J=5.3 Hz, 1H), 7.41 (s, 1H), 7.12 (s, 1H), 4.36 (dd, J=6.7, 5.9 Hz, 2H), 3.94-3.85 (m, 2H), 3.62 (dd, J=7.4, 5.9 Hz, 2H), 3.17 (s, 3H), 2.99 (s, 3H), 2.86-2.76 (m, 1H), 2.53-2.35 (m, 1H).

### Example 24: (R)-3-Hydroxy-1-methyl-3-(3-(3-(2-((2-methylpyridin-3-yl)amino)pyrimidin-4-yl)phenyl)isoxazol-5-yl)pyrrolidin-2-one

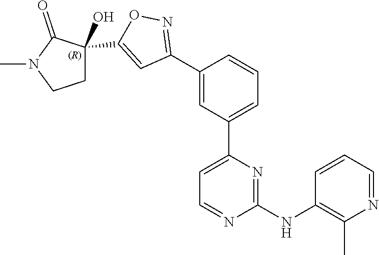

Step A: 4-Chloro-N-(2-methylpyridin-3-yl)pyrimidin-2-amine. 2-Methylpyridin-3-amine (400 mg, 3.70 mmol), and THF (10 mL) were add to an oven-dried and nitrogen-purged 50 mL three-necked round-bottomed flask. This mixture was cooled to −72° C. in an EtOH/dry ice bath, and the resulting mixture treated with LDA (2.2 mL, 2 M in THF, 4.4 mmol), portion-wise over 3 min. The resulting mixture was stirred for 30 min at −78° C. (dry ice/EtOH), treated with 4-chloro-2-(methylsulfonyl)pyrimidine (400 mg, 2.08 mmol) in THF (5 mL), and stirred for 1 h at r.t. After this time, the mixture was poured into water (10 mL) and extracted with ethyl acetate (15 mL×3). The combined organic extracts were washed with brine (30 mL), dried with anhydrous Na₂SO₄, filtered, and concentrated to dryness in vacuo to give a yellow viscous oil. The oil was then subjected to silica gel chromatography (0-100% EtOAc/pet ether) to give 4-chloro-N-(2-methylpyridin-3-yl)pyrimidin-2-amine as a white solid (490 mg, 63%). LCMS (ESI): Mass calcd. For C₁₀H₉ClN₄ 220.05 m/z, found 221.0 [M+H]⁺.

Step B: (R)-3-Hydroxy-1-methyl-3-(3-(3-(2-((2-methylpyridin-3-yl)amino)pyrimidin-4-yl)phenyl)isoxazol-5-yl)pyrrolidin-2-one. (R)-3-Hydroxy-1-methyl-3-(3-(3-(4,4,5,5-tetramethyl-1,3,2-dioxaborolan-2-yl)phenyl)isoxazol-5-yl)pyrrolidin-2-one (Intermediate 11, 250 mg, 0.651 mmol), 4-chloro-N-(2-methylpyridin-3-yl)pyrimidin-2-amine (200 mg, 0.906 mmol), K₃PO₄ (415 mg, 1.96 mmol), and 1,4-dioxane/H₂O (4:1) (8 mL) were added to a nitrogen-purged 40 mL reaction vessel, which was subsequently evacuated and refilled with argon (×3), treated with Pd(dtbpf)Cl₂ (55 mg, 0.084 mmol), and heated for 1 h at 80° C. The reaction vessel was removed from the oil bath and allowed to gradually cool to r.t. The mixture was concentrated to dryness to afford a nearly black viscous oil. This oil was then subjected to HPLC (Xtimate C18 column, 10 μm, 150×40 mm; 20-50% ACN/H₂O with 0.05% NH₃·H₂O) to give, after lyophilization, title compound as a white solid (99 mg, 34%). LC-MS (ESI): Mass calcd. for C₂₄H₂₂N₆O₃ 442.18 m/z, found 443.2 [M+H]⁺. ¹H NMR (400 MHz, DMSO-d₆) δ 9.14 (s, 1H), 8.57 (s, 1H), 8.53 (d, J=5.2 Hz, 1H), 8.27-8.23 (m, 1H), 8.21 (d, J=7.9 Hz, 1H), 8.03 (d, J=7.7 Hz, 1H), 7.99 (d, J=8.0 Hz, 1H), 7.70-7.63 (m, 1H), 7.54 (d, J=5.1 Hz, 1H), 7.29-7.25 (m, 1H), 7.10 (s, 1H), 6.76 (s, 1H), 3.50-3.41 (m, 2H), 2.85 (s, 3H), 2.60-2.55 (m, 1H), 2.48 (s, 3H), 2.35-2.25 (m, 1H).

### Example 25: (R)-3-Hydroxy-1-methyl-3-(3-(3-(2-((1-methyl-1H-pyrazol-3-yl)amino)pyrimidin-4-yl)phenyl)isoxazol-5-yl)pyrrolidin-2-one

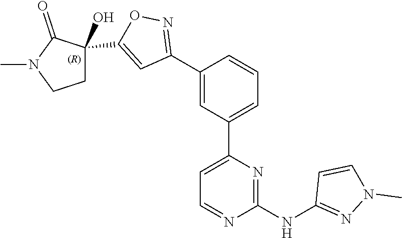

Step A: 2-((1-Methyl-1H-pyrazol-3-yl)amino)pyrimidin-4(1H)-one. 2-(Methylthio)pyrimidin-4(3H)-one (1 g, 7 mmol) and 1-methyl-1H-pyrazol-3-amine (1.0 mL, 12 mmol) were combined. The resultant mixture was heated at 170° C. for 30 min before a yellow solid formed. After this time, the reaction vessel was removed from the oil bath and allowed to gradually cool to r.t. The solid was triturated with ethanol (10 mL) and filtered to afford 2-((1-methyl-1H-pyrazol-3-yl)amino)pyrimidin-4(1H)-one as a colorless solid (1.1 g, 82%). LCMS (ESI): Mass calcd. for C₈H₉N₅O 191.08 m/z, found 191.8 [M+H]⁺.

Step B: 4-Chloro-N-(1-methyl-1H-pyrazol-3-yl)pyrimidin-2-amine. Phosphorus oxychloride (8.5 mL) and 2-((1-methyl-1H-pyrazol-3-yl)amino)pyrimidin-4(1H)-one (500 mg, 2.62 mmol) were combined. The resulting mixture was heated at 100° C. for 2 h. After this time, the reaction vessel was removed from the oil bath and allowed to gradually cool to r.t. The mixture was concentrated to dryness in vacuo, and the residue suspended in H₂O (10 mL), treated with NaHCO₃ (saturated aqueous, 10 mL), and extracted with DCM (10 mL×2). The combined organic extracts were dried over anhydrous Na₂SO₄, filtered, and concentrated to dryness in vacuo to give 4-chloro-N-(1-methyl-1H-pyrazol-3-yl)pyrimidin-2-amine as a colorless solid (510 mg, 87%). LCMS (ESI): Mass calcd. for C₈H₈ClN₅ 209.05 m/z, found 210.1 [M+H]⁺.

Step C: (R)-3-Hydroxy-1-methyl-3-(3-(3-(2-((1-methyl-1H-pyrazol-3-yl)amino)pyrimidin-4-yl)phenyl)isoxazol-5-yl)pyrrolidin-2-one. 4-Chloro-N-(1-methyl-1H-pyrazol-3-yl)pyrimidin-2-amine (100 mg, 0.477 mmol), (R)-3-hydroxy-1-methyl-3-(3-(3-(4,4,5,5-tetramethyl-1,3,2-dioxaborolan-2-yl)phenyl)isoxazol-5-yl)pyrrolidin-2-one (Intermediate 11, 183 mg, 0.477 mmol), K₃PO₄ (304 mg, 1.43 mmol), and 1,4-dioxane/H₂O (4:1) (2 mL) were added to an oven-dried and N₂-purged 5 mL microwave tube, treated with Pd(dtbpf)Cl₂ (31 mg, 0.048 mmol) and heated for 1 hour at 80° C. via microwave irradiation. After this time, the reaction vessel was removed from the microwave reactor and allowed to gradually cool to r.t. The reaction mixture was then diluted with H₂O (50 mL) and extracted with EtOAc (50 mL×3). The combined organic extracts were washed with brine (50 mL), dried over anhydrous Na₂SO₄, filtered, and concentrated to dryness in vacuo to give a brown oil. The oil was subjected to HPLC (Welch Xtimate C18 column, 5 μm, 150×30 mm; 30-60% MeCN/water with 0.05% NH₃·H₂O+10 mM NH₄HCO₃) to give, after lyophilization, the title compound as a colorless solid (95.7 mg, 45%). LCMS (ESI): Mass calcd. for C₂₂H₂₁N₇O₃ 431.17 m/z, found 432.2 [M+H]⁺. ¹H NMR (400 MHz, DMSO-d₆) δ 9.81 (s, 1H), 8.66-8.60 (m, 1H), 8.54 (d, J=5.0 Hz, 1H), 8.27 (d, J=8.0 Hz, 1H), 8.04 (d, J=7.8 Hz, 1H), 7.73-7.65 (m, 1H), 7.63-7.58 (m, 1H), 7.50 (d, J=5.3 Hz, 1H), 7.13 (s, 1H), 6.75 (s, 1H), 6.70-6.66 (m, 1H), 3.77 (s, 3H), 3.53-3.41 (m, 2H), 2.85 (s, 3H), 2.62-2.55 (m, 1H), 2.32-2.24 (m, 1H).

### Example 26: (R)-3-Hydroxy-1-methyl-3-(3-(6-(2-((1-methyl-1H-pyrazol-5-yl)amino)pyrimidin-4-yl)pyridin-2-yl)isoxazol-5-yl)pyrrolidin-2-one

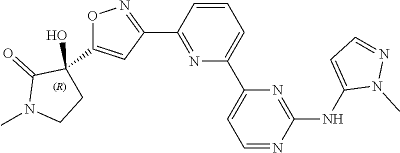

Step A: 4-Chloro-N-(1-methyl-1H-pyrazol-5-yl)pyrimidin-2-amine. 4-Chloro-2-(methylsulfonyl)pyrimidine (530 mg, 2.75 mmol), 1-methyl-1H-pyrazol-5-amine (294 mg, 3.03 mmol), and anhydrous THF (15 mL) were add to a 50 mL round-bottomed flask under an atmosphere of nitrogen. LiHMDS (5.5 mL, 1.0 M in THF, 5.5 mmol) was added and the resulting mixture stirred for 2 h at room temperature. After this time, the mixture was quenched with 1 mL aq. saturated NH₄Cl and then concentrated to dryness in vacuo. The residue was subjected to silica gel chromatography (0-70% EtOAc/pet ether) to give 4-chloro-N-(1-methyl-1H-pyrazol-5-yl)pyrimidin-2-amine as a light yellow solid (510 mg, 88%). ¹H NMR (400 MHz, CDCl₃) δ 8.21 (d, J=5.2 Hz, 1H), 7.42 (d, J=2.0 Hz, 1H), 7.02 (br s, 1H), 6.75 (d, J=5.2 Hz, 1H), 6.23 (d, J=2.0 Hz, 1H), 3.72-3.69 (m, 3H).

Step B: N-(1-Methyl-1H-pyrazol-5-yl)-4-(trimethylstannyl)pyrimidin-2-amine. 4-Chloro-N-(1-methyl-1H-pyrazol-5-yl)pyrimidin-2-amine (360 mg, 1.72 mmol), Pd(PPh₃)₄ (198 mg, 0.172 mmol), 1,1,1,2,2,2-hexamethyldistannane (776 mg, 2.37 mmol), and anhydrous 1,4-dioxane (8 mL) were added to a 50 mL three-necked round-bottomed flask fitted with a reflux condenser, under an atmosphere of nitrogen. The resultant mixture was heated at 100° C. for 16 h and turned brown. After this time, the reaction vessel was removed from the oil bath and allowed to gradually cool to room temperature. The reaction mixture was used in the next step directly. LCMS (ESI): Mass calcd. for C₁₁H₁₇N₅Sn 339.05 m/z, found 340.1 [M+H]⁺.

Step C: (R)-3-Hydroxy-1-methyl-3-(3-(6-(2-((1-methyl-1H-pyrazol-5-yl)amino)pyrimidin-4-yl)pyridin-2-yl)isoxazol-5-yl)pyrrolidin-2-one. (R)-3-(3-(6-Bromopyridin-2-yl)isoxazol-5-yl)-3-hydroxy-1-methylpyrrolidin-2-one (Intermediate 13, Step A, 100 mg, 0.296 mmol), Pd(PPh₃)₄ (34 mg, 0.030 mol), N-(1-methyl-1H-pyrazol-5-yl)-4-(trimethylstannyl)pyrimidin-2-amine (2.8 mL, 0.21 M in dioxane), CuI (5.6 mg, 0.030 mmol), and anhydrous DMF (3 mL) were combined under an atmosphere of nitrogen. The resultant mixture was heated at 100° C. for 16 h and turned brown. After this time, the mixture was allowed to cool to room temperature and concentrated to dryness in vacuo. The residue was subjected to HPLC (Phenomenex C18 column, 75×30 mm, 3 μm, 18-48% (v/v) CH₃CN/H₂O with 0.05% NH₃) to give (R)-3-hydroxy-1-methyl-3-(3-(6-(2-((1-methyl-1H-pyrazol-5-yl)amino)pyrimidin-4-yl)pyridin-2-yl)isoxazol-5-yl)pyrrolidin-2-one as a white solid (70.8 mg, 54%). LCMS (ESI): Mass calcd. for C₂₁H₂₀N₈O₃ 432.17 m/z, found 433.2 [M+H]⁺. ¹H NMR (400 MHz, DMSO-d₆) δ 9.64 (s, 1H), 8.70-8.65 (m, 1H), 8.40-8.32 (m, 1H), 8.23-8.11 (m, 2H), 7.95-7.90 (m, 1H), 7.41-7.80 (m, 1H), 7.18 (s, 1H), 6.78 (s, 1H), 6.38-6.33 (m, 1H), 3.73 (s, 3H), 3.55-3.41 (m, 2H), 2.85 (s, 3H), 2.68-2.58 (m, 1H), 2.35-2.24 (m, 1H).

### Example 27: (R)-4,4-Difluoro-3-hydroxy-1-methyl-3-(3-(6-(2-((1-methyl-1H-pyrazol-3-yl)amino)pyrimidin-4-yl)pyridin-2-yl)isoxazol-5-yl)pyrrolidin-2-one

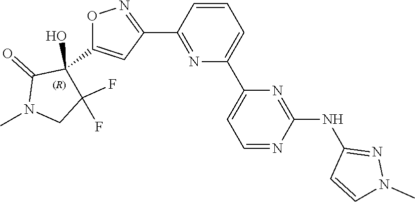

Step A: (E)-6-Chloropicolinaldehyde oxime. 6-Chloropicolinaldehyde (5.0 g, 35.3 mmol), hydroxylamine hydrochloride (3.19 g, 45.9 mmol), sodium acetate (5.80 g, 70.6 mmol) and EtOH (20 mL) were added to a 250 mL three neck flask. The resulting mixture was heated at 45° C. for 2 h. After this time, the reaction mixture was cooled to room temperature, filtered by suction, and the filter cake was washed with ethanol (30 mL×3). The filtrates were combined, and the solvent was evaporated under reduced pressure. The residue was dissolved in ethyl acetate (30 mL), washed with water (20 mL×2), dried over anhydrous sodium sulfate, filtered and concentrated to give 6-chloropicolinaldehyde oxime as white solid (5.0 g, 90%). LCMS (ESI): Mass calcd. for C₆H₅ClN₂O 156.0 m/z, found 156.8 [M+H]⁺.

Step B: 6-Chloro-N-hydroxypicolinimidoyl chloride. 6-Chloropicolinaldehyde oxime (4.5 g, 29 mmol), and DMF (15 mL) were added to a 50 mL round-bottomed flask, and NCS (4.5 g, 34 mmol) was added in 3 batches. The resulting mixture was stirred for 16 h at r.t. The reaction was then diluted with water (30 mL) and extracted with ethyl acetate (25 mL×3). The combined extracts were washed with brine (20 mL), dried over anhydrous Na₂SO₄, filtered, and concentrated to dryness in vacuo to give a yellow solid. The yellow solid was then subjected to silica gel chromatography (0-25% ethyl acetate/petroleum ether) to give 6-chloro-N-hydroxypicolinimidoyl chloride as a yellow solid (6.2 g, 95%). LC-MS (ESI): Mass calcd. for C₆H⁴Cl₂N₂O 189.97 m/z found 190.8 [M+H]⁺. ¹H NMR (400 MHz, DMSO-d₆) δ 12.96 (s, 1H), 8.00-7.95 (m, 1H), 7.91-7.87 (m, 1H), 7.66-7.62 (m, 1H).

Step C: (R)-3-(3-(6-chloropyridin-2-yl)isoxazol-5-yl)-4,4-difluoro-3-hydroxy-1-methylpyrrolidin-2-one. 6-Chloro-N-hydroxypicolinimidoyl chloride (400 mg, 2.09 mmol), (R)-3-ethynyl-4,4-difluoro-3-hydroxy-1-methylpyrrolidin-2-one (Intermediate 2, 368 mg, 2.10 mmol), TEA (0.50 mL, 4.0 mmol), and DCM (8 mL) were added to a 25 mL round-bottomed flask. The resulting mixture stirred for 16 h at room temperature. The reaction was concentrated to dryness in vacuo to give a yellow solid. The yellow solid was then subjected to silica gel chromatography (0-70% ethyl acetate/petroleum ether) to give (R)-3-(3-(6-chloropyridin-2-yl)isoxazol-5-yl)-4,4-difluoro-3-hydroxy-1-methylpyrrolidin-2-one as a colorless oil (500 mg, 40%). LC-MS (ESI): Mass calcd. for C₁₃H₁₀ClF₂N₃O₃ 329.04 m/z found 330.0 [M+H]⁺.

Step D: (R)-4,4-Difluoro-3-hydroxy-1-methyl-3-(3-(6-(2-((1-methyl-1H-pyrazol-3-yl)amino)pyrimidin-4-yl)pyridin-2-yl)isoxazol-5-yl)pyrrolidin-2-one. (R)-3-(3-(6-chloropyridin-2-yl)isoxazol-5-yl)-4,4-difluoro-3-hydroxy-1-methylpyrrolidin-2-one (200 mg, 0.607 mmol), N-(1-methyl-1H-pyrazol-3-yl)-4-(tributylstannyl)pyrimidin-2-amine (Intermediate 24, 204 mg, 0.604 mmol) and 1,4-dioxane (6 mL) were added to 50 mL round-bottomed flask. Pd(dppf)Cl₂ (88 mg, 0.12 mmol) was then added. The resulting mixture was heated for 1 h at 100° C. The reaction vessel was removed from the heating mantle and allowed to gradually cool to r.t. The reaction was then diluted with water (4 mL), extracted with ethyl acetate (8 mL×3), and the combined extracts washed with brine (6 mL), dried over anhydrous Na₂SO₄, filtered, and concentrated to dryness in vacuo to give a yellow solid. The yellow solid was then subjected to HPLC (Welch Xtimate C18 5 μm, 150×30 mm column (Isocratic: 28-58% ACN (v/v)/water (NH₃H₂O+NH₄HCO₃)) to give the title compound as a yellow solid (6.8 mg, 2%). LC-MS (ESI): Mass calcd. for C₂₁H₁₈F₂N₈O₃ 468.2 m/z found 469.2 [M+H]⁺. ¹H NMR (400 MHz, DMSO-d₆) δ 9.90 (s, 1H), 8.67-8.62 (m, 1H), 8.52-8.45 (m, 1H), 8.26-8.15 (m, 2H), 7.99 (s, 1H), 7.87-7.83 (m, 1H), 7.65-7.59 (m, 1H), 7.32 (s, 1H), 6.74-6.71 (m, 1H), 4.08-3.98 (m, 2H), 3.78 (s, 3H), 2.97 (s, 3H).

### Example 28: (R)-3-(3-(6-(2-((3-Cyclopropoxy-1-(2,2-difluoroethyl)-1H-pyrazol-4-yl)amino)pyrimidin-4-yl)pyridin-2-yl)isoxazol-5-yl)-3-hydroxy-1-methylpyrrolidin-2-one

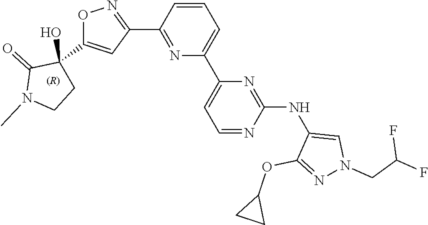

(R)-3-Hydroxy-1-methyl-3-(3-(6-(2-(methylsulfonyl)pyrimidin-4-yl)pyridin-2-yl)isoxazol-5-yl)pyrrolidin-2-one (Intermediate 13, 120 mg, 0.289 mmol) and 1,4-dioxane (5 mL) were added to a 40 mL flask, which was charged with 3-cyclopropoxy-1-(2,2-difluoroethyl)-1H-pyrazol-4-amine (Intermediate 14, 275 mg, 1.35 mmol) and TsOH (60 mg, 0.35 mmol). The resultant mixture was heated at 115° C. for 16 h. The reaction vessel was removed from the oil bath and allowed to gradually cool to r.t. The reaction mixture was then diluted with DCM (20 mL), washed with aq. NaOH (1 M, 10 mL×3), dried over anhydrous Na₂SO₄, filtered, and concentrated to dryness in vacuo to give a black oil. The oil was then subjected to HPLC (Welch Xtimate C18 column, 5 μm, 150×30 mm; 35-65% ACN/water with 0.05% NH₃·H₂O+10 mM NH₄HCO₃), and then subjected to SFC (DAICEL CHIRALPAK® AD column, 10 μm, 250×30 mm; 55% (v/v) IPA containing 0.1% of 25% aq. NH₃)/CO₂) to give (R)-3-(3-(6-(2-((3-cyclopropoxy-1-(2,2-difluoroethyl)-1H-pyrazol-4-yl)amino)pyrimidin-4-yl)pyridin-2-yl)isoxazol-5-yl)-3-hydroxy-1-methylpyrrolidin-2-one as a yellow solid (57.62 mg, 34%). LCMS (ESI): Mass calcd. for C₂₅H₂₄F₂N₈O₄ 538.51 m/z found 539.2 [M+H]⁺. ¹H NMR (400 MHz, DMSO-d₆) δ 8.67 (br s, 1H), 8.59 (d, J=5.2 Hz, 1H), 8.42 (br s, 1H), 8.19-8.13 (m, 2H), 7.95 (br s, 1H), 7.77 (d, J=5.2 Hz, 1H), 7.17 (s, 1H), 6.95-6.60 (m, 1H), 6.52-6.16 (m, 1H), 4.62-4.41 (m, 2H), 4.17-4.06 (m, 1H), 3.54-3.45 (m, 2H), 2.85 (s, 3H), 2.66-2.58 (m, 1H), 2.32-2.26 (m, 1H), 0.72-0.63 (m, 4H).

### Example 29: (R)-3-(3-(6-(2-((1,3-Dimethyl-1H-pyrazol-4-yl)amino)pyrimidin-4-yl)pyridin-2-yl)isoxazol-5-yl)-3-hydroxy-1-methylpyrrolidin-2-one

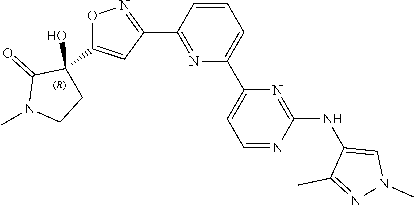

(R)-3-Hydroxy-1-methyl-3-(3-(6-(2-(methylsulfonyl)pyrimidin-4-yl)pyridin-2-yl)isoxazol-5-yl)pyrrolidin-2-one (Intermediate 13, 100 mg, 0.241 mmol) and 1,4-dioxane (4 mL) were added to a 40 mL flask, which was charged with 1,3-dimethyl-1H-pyrazol-4-amine (240 μL, 2.53 mmol) and TsOH (50 mg, 0.29 mmol). The mixture was heated at 115° C. for 16 h. The reaction vessel was removed from the heating mantle and allowed to gradually cool to r.t. The reaction mixture was diluted with DCM (30 mL), washed with aqueous NaOH (2 N, 30 mL), and brine (30 mL), dried over anhydrous MgSO₄, filtered, and concentrated to dryness in vacuo to give a green solid. The solid was subjected to HPLC (Boston Prime C18 column, 5 μm, 150×30 mm; 25-55% ACN/water with 0.05% NH₃·H₂O+10 mM NH₄HCO₃) to give (R)-3-(3-(6-(2-((1,3-dimethyl-1H-pyrazol-4-yl)amino)pyrimidin-4-yl)pyridin-2-yl)isoxazol-5-yl)-3-hydroxy-1-methylpyrrolidin-2-one as a yellow solid (59.8 mg, 52%). LCMS (ESI): Mass calcd. for C₂₂H₂₂N₈O₃ 446.18 m/z found 447.2 [M+H]⁺. ¹H NMR (400 MHz, DMSO-d₆) δ 8.95 (br s, 1H), 8.60 (d, J=5.2 Hz, 1H), 8.47-8.39 (m, 1H), 8.23-8.12 (m, 2H), 7.91 (br s, 1H), 7.75 (d, J=5.2 Hz, 1H), 7.17 (s, 1H), 6.79 (s, 1H), 3.79 (s, 3H), 3.54-3.45 (m, 2H), 2.86 (s, 3H), 2.66-2.59 (m, 1H), 2.33-2.26 (m, 1H), 2.15 (s, 3H).

### Example 30: (R)-3-Hydroxy-1-methyl-3-(5-(6-(2-((1-methyl-1H-pyrazol-4-yl)amino)pyrimidin-4-yl)pyridin-2-yl)isoxazol-3-yl)pyrrolidin-2-one

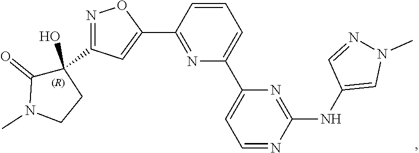

(R)-3-Hydroxy-1-methyl-3-(5-(6-(2-(methylsulfonyl)pyrimidin-4-yl)pyridin-2-yl)isoxazol-3-yl)pyrrolidin-2-one (Intermediate 21, 300 mg, 0.722 mmol), 1-methyl-1H-pyrazol-4-amine (140 mg, 1.44 mmol), DMSO (anhydrous, 2.4 mL), and TFA (0.17 mL, 2.16 mmol) were combined and the resulting reaction mixture was heated at 150° C. for 90 min. After the reaction mixture cooled to room temperature, it was filtered through a syringe filter and purified by RP-HPLC (Welch Xtimate C18 column, 5 μm, 150×30 mm; 35-65% ACN/water with 0.05% NH₃·H₂O+10 mM NH₄HCO₃) to give a yellow solid (50 mg, 16%). ¹H NMR (400 MHz, Methanol-d₄) δ 8.51 (d, J=5.6 Hz, 1H), 8.48 (d, J=7.8 Hz, 1H), 8.13 (t, J=7.8 Hz, 1H), 8.05 (dd, J=7.8, 1.1 Hz, 1H), 8.03-7.98 (m, 1H), 7.89 (d, J=5.6 Hz, 1H), 7.68 (s, 1H), 7.30 (s, 1H), 3.94 (s, 3H), 3.62-3.51 (m, 2H), 2.94 (s, 3H), 2.88 (dt, J=13.2, 5.0 Hz, 1H), 2.39 (dt, J=13.2, 7.9 Hz, 1H). LC-MS (ESI): Mass calcd. for C₂₁H₂₀N₈O₃ 432.2 m/z, found 433.0 [M+H]⁺.

### Example 31: (R)-3-(3-(6-(2-((1H-Pyrazol-3-yl)amino)pyrimidin-4-yl)pyridin-2-yl)isoxazol-5-yl)-3-hydroxy-1-methylpyrrolidin-2-one

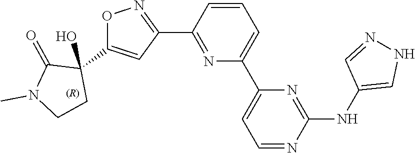

(R)-3-Hydroxy-1-methyl-3-(3-(6-(2-(methylsulfonyl)pyrimidin-4-yl)pyridin-2-yl)isoxazol-5-yl)pyrrolidin-2-one (Intermediate 13, 60 mg, 0.144 mmol) and DMSO (0.48 mL) were added to a vial, which was charged with tert-butyl 3-amino-1H-pyrazole-1-carboxylate (53 mg, 0.29 mmol) and TFA (30 μL, 0.36 mmol). The mixture was heated at 150° C. for 75 min. The reaction vessel was removed from the heating mantle and allowed to gradually cool to r.t. The reaction mixture was filtered through a syringe filter and directly subjected to reverse-phase HPLC purification using acidic media and column C1 to provide the title compound as its TFA salt as a yellow solid (13 mg, 16%). LC-MS (ESI): Mass calcd. for C₂₀H₁₈N₈O₃ 418.2 m/z found 418.8 [M+H]⁺. ¹H NMR (500 MHz, Methanol-d₄) δ 8.88 (d, J=5.8 Hz, 1H), 8.77 (d, J=7.8 Hz, 1H), 8.69 (s, 1H), 8.45-8.32 (m, 1H), 8.25-8.10 (m, 2H), 7.21 (s, 1H), 6.14 (s, 1H), 3.65-3.52 (m, 2H), 2.98 (s, 3H), 2.77 (ddd, J=12.4, 6.9, 4.7 Hz, 1H), 2.43 (dq, J=13.8, 7.1, 6.6 Hz, 1H). The TFA salt could be converted to the corresponding free base by passage through a carbonate cartridge.

### Example 32: (R)-3-(3-(6-(2-(3-Amino-1H-pyrazol-1-yl)pyrimidin-4-yl)pyridin-2-yl)isoxazol-5-yl)-3-hydroxy-1-methylpyrrolidin-2-one

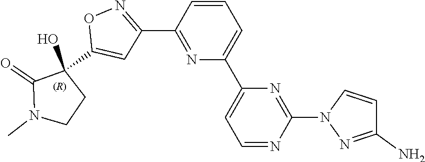

The title compound was made in a manner analogous to Example 31. The compound was purified by reverse-phase HPLC purification using acidic media and column C1 to provide the title compound (5%) as its TFA salt. ¹H NMR (500 MHz, Methanol-d₄) δ 9.03 (d, J=5.2 Hz, 1H), 8.77 (dd, J=7.8, 1.1 Hz, 1H), 8.53 (dd, J=5.2, 0.8 Hz, 1H), 8.24 (dd, J=7.8, 1.1 Hz, 1H), 8.17 (t, J=7.8 Hz, 1H), 7.95-7.89 (m, 1H), 7.22 (s, 1H), 5.87 (d, J=2.7 Hz, 1H), 3.64-3.55 (m, 2H), 2.98 (s, 3H), 2.78 (ddd, J=13.6, 7.2, 4.9 Hz, 1H), 2.43 (ddd, J=13.5, 8.0, 6.4 Hz, 1H). LC-MS (ESI): Mass calcd. for C₂₀H₁₈N₈O₃ 418.2 m/z found 418.8 [M+H]⁺. The TFA salt could be converted to the corresponding free base by passage through a carbonate cartridge.

### Example 33: (R)-3-Hydroxy-1-methyl-3-(3-(6-(2-((3-(trifluoromethyl)-1H-pyrazol-4-yl)amino)pyrimidin-4-yl)pyridin-2-yl)isoxazol-5-yl)pyrrolidin-2-one

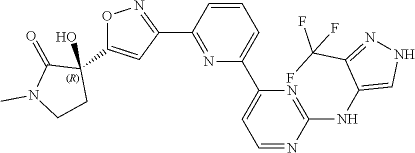

The title compound was made in a manner analogous to Example 31. The compound was purified by reverse-phase HPLC purification using acidic media and column C1 to provide the title compound (15%) as its TFA salt. ¹H NMR (600 MHz, Methanol-d₄) δ 8.97 (d, J=5.1 Hz, 1H), 8.79 (d, J=7.8 Hz, 1H), 8.53 (d, J=5.1 Hz, 1H), 8.43 (s, 1H), 8.26-8.21 (m, 1H), 8.16 (t, J=7.8 Hz, 1H), 7.23 (s, 1H), 3.62-3.57 (m, 1H), 2.98 (s, 3H), 2.81-2.75 (m, 1H), 2.46-2.40 (m, 1H). LC-MS (ESI): Mass calcd. for C₂₁H₁₇F₃N₈O₃ 486.1 m/z found 487.2 [M+H]⁺. The TFA salt could be converted to the corresponding free base by passage through a carbonate cartridge.

### Example 34: (R)-3-Hydroxy-1-methyl-3-(3-(6-(2-((4-methyl-1H-pyrazol-5-yl)amino)pyrimidin-4-yl)pyridin-2-yl)isoxazol-5-yl)pyrrolidin-2-one

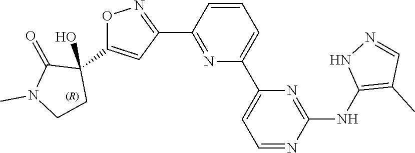

The title compound was made in a manner analogous to Example 31. The compound was purified by reverse-phase HPLC purification using acidic media and column C1 to provide the title compound (33%) as its TFA salt. ¹H NMR (600 MHz, Methanol-d₄) δ 8.85 (d, J=5.1 Hz, 1H), 8.76 (dd, J=7.8, 1.1 Hz, 1H), 8.47 (d, J=1.2 Hz, 1H), 8.32 (d, J=5.1 Hz, 1H), 8.20 (dd, J=7.8, 1.1 Hz, 1H), 8.13 (t, J=7.8 Hz, 1H), 7.21 (s, 1H), 3.64-3.55 (m, 2H), 2.98 (s, 3H), 2.77 (ddd, J=13.6, 7.2, 4.8 Hz, 1H), 2.43 (ddd, J=14.0, 8.1, 6.4 Hz, 1H). Mass calcd. for C₂₁H₂₀N₈O₃ 432.2 m/z found 433.8 [M+H]⁺. The TFA salt could be converted to the corresponding free base by passage through a carbonate cartridge.

### Example 35: (R)-3-(3-(6-(2-(5-Amino-4-methyl-1H-pyrazol-1-yl)pyrimidin-4-yl)pyridin-2-yl)isoxazol-5-yl)-3-hydroxy-1-methylpyrrolidin-2-one

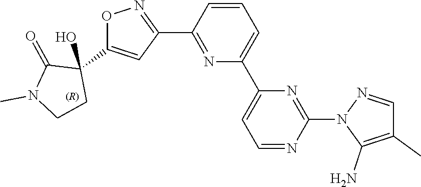

The title compound was made in a manner analogous to Example 31. The compound was purified by reverse-phase HPLC purification using acidic media and column C1 to provide the title compound (11%) as its TFA salt. ¹H NMR (600 MHz, Methanol-d₄) δ 9.04 (d, J=5.1 Hz, 1H), 8.77 (dd, J=7.8, 1.1 Hz, 1H), 8.54 (d, J=5.1 Hz, 1H), 8.27-8.23 (m, 1H), 8.17 (t, J=7.8 Hz, 2H), 7.84 (s, 1H), 7.22 (s, 1H), 3.63-3.56 (m, 2H), 2.98 (s, 3H), 2.81-2.75 (m, 1H), 2.47-2.41 (m, 1H), 2.07 (s, 3H). Mass calcd. for C₂₁H₁₇F₃N₈O₃ 432.2 m/z found 433.4 [M+H]⁺. The TFA salt could be converted to the corresponding free base by passage through a carbonate cartridge.

### Example 36: (R)-3-Hydroxy-1-methyl-3-(3-(6-(2-((3-methyl-1H-pyrazol-4-yl)amino)pyrimidin-4-yl)pyridin-2-yl)isoxazol-5-yl)pyrrolidin-2-one

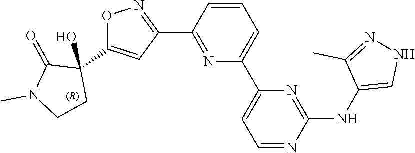

The title compound was made in a manner analogous to Example 31. The compound was purified by reverse-phase HPLC purification using acidic media and column C1 to provide the title compound (10%) as its TFA salt. ¹H NMR (600 MHz, Methanol-d₄) δ 8.49 (d, J=5.7 Hz, 2H), 8.19 (d, J=7.8 Hz, 1H), 8.10 (t, J=7.9 Hz, 1H), 8.02-7.96 (m, 2H), 7.19 (s, 1H), 3.64-3.55 (m, 2H), 2.97 (s, 3H), 2.77 (ddd, J=13.5, 7.3, 4.7 Hz, 1H), 2.42 (ddd, J=13.6, 8.1, 6.3 Hz, 1H), 2.33 (s, 3H). Mass calcd. for C₂₁H₂ON₈O₃ 432.2 m/z found 433.2 [M+H]⁺. The TFA salt could be converted to the corresponding free base by passage through a carbonate cartridge.

### Example 37: (R)-3-(3-(6-(2-(4-Amino-3-methyl-1H-pyrazol-1-yl)pyrimidin-4-yl)pyridin-2-yl)isoxazol-5-yl)-3-hydroxy-1-methylpyrrolidin-2-one

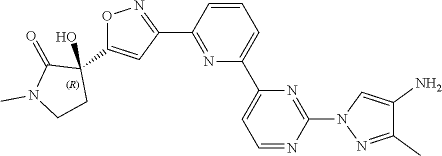

The title compound was made in a manner analogous to Example 31. The compound was purified by reverse-phase HPLC purification using acidic media and column C1 to provide the title compound (5%) as its TFA salt. ¹H NMR (600 MHz, Methanol-d₄) δ 8.96 (d, J=5.1 Hz, 1H), 8.81-8.77 (m, 2H), 8.50 (d, J=5.1 Hz, 1H), 8.23 (dd, J=7.8, 1.1 Hz, 1H), 8.16 (t, J=7.8 Hz, 1H), 7.22 (s, 1H), 3.62-3.57 (m, 2H), 2.98 (s, 3H), 2.78 (ddd, J=13.5, 7.3, 4.7 Hz, 1H), 2.46-2.39 (m, 4H). Mass calcd. for C₂₁H₂ON₈O₃ 432.2 m/z found 433.2 [M+H]⁺. The TFA salt could be converted to the corresponding free base by passage through a carbonate cartridge.

### Example 38: (R)-3-Hydroxy-1-methyl-3-(3-(6-(2-(pyrazolo[1,5-a]pyrimidin-3-ylamino)pyrimidin-4-yl)pyridin-2-yl)isoxazol-5-yl)pyrrolidin-2-one

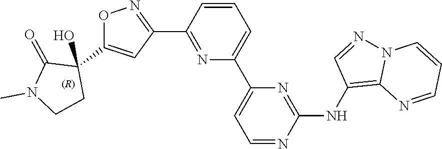

The title compound was made in a manner analogous to Example 31. The compound was purified by reverse-phase HPLC purification using acidic media and column C1 to provide the title compound (9%) as its TFA salt. ¹H NMR (600 MHz, Methanol-d₄) δ 8.90 (dd, J=7.1, 1.6 Hz, 1H), 8.82-8.73 (m, 1H), 8.64 (s, 1H), 8.54-8.50 (m, 2H), 8.18 (d, J=7.9 Hz, 1H), 8.10 (t, J=7.8 Hz, 1H), 7.99 (d, J=5.4 Hz, 1H), 7.19 (s, 2H), 7.03 (dd, J=7.1, 4.0 Hz, 1H), 3.63-3.56 (m, 2H), 2.98 (s, 3H), 2.77 (ddd, J=13.5, 7.2, 4.7 Hz, 1H), 2.42 (ddd, J=13.5, 8.1, 6.4 Hz, 1H). Mass calcd. for C₂₃H₁₉N₉O₃ 469.1 m/z found 470.2 [M+H]⁺. The TFA salt could be converted to the corresponding free base by passage through a carbonate cartridge.

### Example 39: (R)-3-(3-(6-(2-(4-Amino-1H-pyrazol-1-yl)pyrimidin-4-yl)pyridin-2-yl)isoxazol-5-yl)-3-hydroxy-1-methylpyrrolidin-2-one

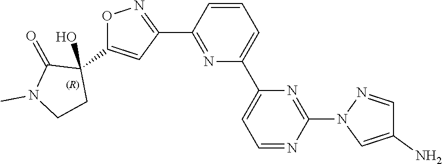

The title compound was made in a manner analogous to Example 31. The compound was purified by reverse-phase HPLC purification using acidic media and column C1 to provide the title compound (3%) as its TFA salt. ¹H NMR (600 MHz, Methanol-d₄) δ 8.98 (d, J=5.1 Hz, 1H), 8.82 (d, J=0.9 Hz, 1H), 8.78 (dd, J=7.8, 1.1 Hz, 1H), 8.53 (d, J=5.1 Hz, 1H), 8.23 (dd, J=7.8, 1.0 Hz, 1H), 8.16 (t, J=7.8 Hz, 1H), 7.91 (d, J=0.9 Hz, 1H), 7.22 (s, 1H), 3.65-3.54 (m, 2H), 2.98 (s, 3H), 2.78 (ddd, J=13.6, 7.3, 4.7 Hz, 1H), 2.43 (ddd, J=13.5, 8.1, 6.4 Hz, 1H). Mass calcd. for C₂₀H₁₈N₈O₃ 418.2 m/z found 487.2 [M+H]⁺. The TFA salt could be converted to the corresponding free base by passage through a carbonate cartridge.

### Example 40: (R)-3-(3-(6-(2-((1H-Pyrazol-4-yl)amino)pyrimidin-4-yl)pyridin-2-yl)isoxazol-5-yl)-3-hydroxy-1-methylpyrrolidin-2-one

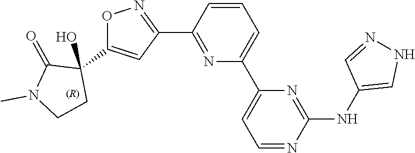

The title compound was made in a manner analogous to Example 31. The compound was purified by reverse-phase HPLC purification using acidic media and column C1 to provide the title compound (33%) as its TFA salt. ¹H NMR (600 MHz, Methanol-d₄) δ 8.55 (t, J=6.6 Hz, 2H), 8.18 (d, J=7.9 Hz, 1H), 8.11 (t, J=7.7 Hz, 1H), 8.01-7.96 (m, 2H), 7.95-7.87 (m, 2H), 7.19 (s, 1H), 3.63-3.56 (m, 2H), 2.98 (s, 3H), 2.81-2.74 (m, 1H), 2.45-2.38 (m, 1H). Mass calcd. for C₂₀H₁₈N₈O₃ 418.2 m/z found 487.2 [M+H]⁺. The TFA salt could be converted to the corresponding free base by passage through a carbonate cartridge.

### Example 41: (R)-3-Hydroxy-1-methyl-3-(3-(6-(2-((3-methylisoxazol-4-yl)amino)pyrimidin-4-yl)pyridin-2-yl)isoxazol-5-yl)pyrrolidin-2-one

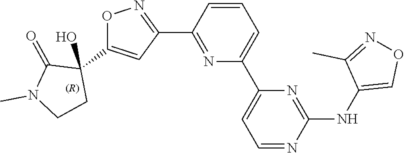

The title compound was made in a manner analogous to Example 31. The compound was purified by reverse-phase HPLC purification using acidic media and column C1 to provide the title compound (39%) as its TFA salt. ¹H NMR (600 MHz, Methanol-d₄) δ 9.09 (s, 1H), 8.63 (d, J=5.1 Hz, 1H), 8.50 (d, J=7.8 Hz, 1H), 8.15 (dd, J=7.8, 1.1 Hz, 1H), 8.09 (t, J=7.8 Hz, 1H), 7.94 (d, J=5.1 Hz, 1H), 7.18 (s, 1H), 3.64-3.53 (m, 2H), 2.98 (s, 3H), 2.77 (ddd, J=13.5, 7.2, 4.8 Hz, 1H), 2.42 (ddd, J=13.4, 8.0, 6.4 Hz, 1H), 2.38 (d, J=0.6 Hz, 3H). Mass calcd. for C₂₁H₁₉N₇O₄ 433.15 m/z found 434.2 [M+H]⁺. The TFA salt could be converted to the corresponding free base by passage through a carbonate cartridge.

### Example 42: (R)-3-Hydroxy-1-methyl-3-(3-(6-(2-((1-(2,2,2-trifluoroethyl)-1H-pyrazol-4-yl)amino)pyrimidin-4-yl)pyridin-2-yl)isoxazol-5-yl)pyrrolidin-2-one

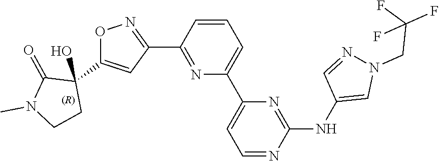

The title compound was made in a manner analogous to Example 31. The compound was purified by reverse-phase HPLC purification using acidic media and column C1 to provide the title compound (40%) as its TFA salt. ¹H NMR (600 MHz, Methanol-d₄) δ 8.58 (d, J=5.2 Hz, 1H), 8.54 (d, J=7.7 Hz, 1H), 8.23 (s, 1H), 8.17 (d, J=6.7 Hz, 1H), 8.10 (t, J=7.8 Hz, 1H), 7.91 (d, J=5.2 Hz, 1H), 7.77 (s, 1H), 7.71 (d, J=0.7 Hz, 1H), 7.19 (s, 1H), 4.95 (dd, J=17.5, 8.7 Hz, 2H), 3.64-3.54 (m, 2H), 2.98 (s, 3H), 2.77 (ddd, J=13.6, 7.2, 4.7 Hz, 1H), 2.47-2.39 (m, 1H). Mass calcd. for C₂₂H₁₉F₃N₈O₃ 500.2 m/z found 501.2 [M+H]⁺. The TFA salt could be converted to the corresponding free base by passage through a carbonate cartridge.

### Example 43: (R)-3-Hydroxy-1-methyl-3-(3-(6-(2-(((R)-1-(1-methyl-1H-pyrazol-3-yl)ethyl)amino)pyrimidin-4-yl)pyridin-2-yl)isoxazol-5-yl)pyrrolidin-2-one

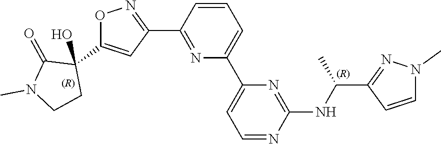

The title compound was made in a manner analogous to Example 66. The compound was purified by reverse-phase HPLC purification using acidic media and column C1. The product was then further purified by SFC (CHIRALCEL® OJ-H, 5 μm, 250×21 mm, Mobile phase: 25% methanol, 75% CO₂) to provide the title compound as the first eluting peak (18%). ¹H NMR (400 MHz, Methanol-d₄) δ 8.48 (dd, J=7.8, 1.2 Hz, 1H), 8.42 (d, J=5.1 Hz, 1H), 8.11 (dd, J=7.8, 1.2 Hz, 1H), 8.03 (t, J=7.8 Hz, 1H), 7.71 (d, J=5.1 Hz, 1H), 7.46 (d, J=2.3 Hz, 1H), 7.16 (s, 1H), 6.25 (d, J=2.3 Hz, 1H), 5.36 (q, J=6.9 Hz, 1H), 3.85 (s, 3H), 3.63-3.55 (m, 2H), 2.97 (s, 3H), 2.76 (ddd, J=13.5, 7.0, 5.0 Hz, 1H), 2.41 (ddd, J=13.4, 8.0, 6.6 Hz, 1H), 1.59 (d, J=6.9 Hz, 3H), 1.17 (t, J=7.1 Hz, 1H). Mass calcd. for C₂₃H₂₄N₈O₃ 460.2 m/z found 461.0 [M+H]⁺.

### Example 44: (R)-3-Hydroxy-1-methyl-3-(3-(6-(2-(((S)-1-(1-methyl-1H-pyrazol-3-yl)ethyl)amino)pyrimidin-4-yl)pyridin-2-yl)isoxazol-5-yl)pyrrolidin-2-one

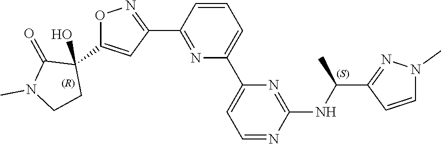

The title compound was isolated as the second eluting peak from the reaction mixture in Example 43 to provide the title compound (18%). ¹H NMR (400 MHz, Methanol-d₄) δ 8.48 (dd, J=7.8, 1.2 Hz, 1H), 8.42 (d, J=5.1 Hz, 1H), 8.11 (dd, J=7.8, 1.2 Hz, 1H), 8.03 (t, J=7.8 Hz, 1H), 7.71 (d, J=5.1 Hz, 1H), 7.46 (d, J=2.3 Hz, 1H), 7.16 (s, 1H), 6.25 (d, J=2.3 Hz, 1H), 5.36 (q, J=6.9 Hz, 1H), 3.85 (s, 3H), 3.65-3.52 (m, 3H), 2.97 (s, 3H), 2.76 (ddd, J=13.5, 6.9, 4.9 Hz, 1H), 2.41 (ddd, J=13.5, 8.0, 6.6 Hz, 1H), 1.60 (d, J=6.9 Hz, 3H), 1.17 (t, J=7.1 Hz, 1H). Mass calcd. for C₂₃H₂₄N₈O₃ 460.2 m/z found 461.0 [M+H]⁺.

### Example 45: (R)-3-Hydroxy-1-methyl-3-(3-(6-(2-((1-(1-methyl-1H-pyrazol-4-yl)cyclopropyl)amino)pyrimidin-4-yl)pyridin-2-yl)isoxazol-5-yl)pyrrolidin-2-one

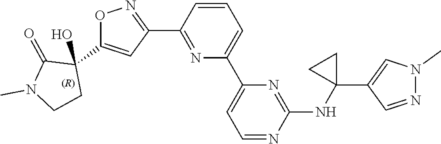

The title compound was made in a manner analogous to Example 66. The compound was purified by reverse-phase HPLC purification using acidic media and column C1 to provide the title compound (6%) as its TFA salt. ¹H NMR (600 MHz, Methanol-d₄) δ 8.55 (d, J=7.9 Hz, 1H), 8.43 (d, J=6.0 Hz, 1H), 8.27-8.20 (m, 1H), 8.18-8.10 (m, 1H), 8.03-7.94 (m, 1H), 7.55 (d, J=0.9 Hz, 1H), 7.39 (s, 1H), 7.18 (s, 1H), 3.80 (s, 3H), 3.64-3.54 (m, 2H), 2.97 (s, 3H), 2.76 (ddd, J=13.5, 7.3, 4.7 Hz, 1H), 2.42 (ddd, J=13.6, 8.2, 6.3 Hz, 1H), 1.43-1.26 (m, 4H). Mass calcd. for C₂₀H₁₈N₈O₃ 472.2 m/z found 473.2 [M+H]⁺. The TFA salt could be converted to the corresponding free base by passage through a carbonate cartridge.

### Example 46: (R)-3-(3-(6-(2-((1-Ethyl-1H-pyrazol-4-yl)amino)pyrimidin-4-yl)pyridin-2-yl)isoxazol-5-yl)-3-hydroxy-1-methylpyrrolidin-2-one

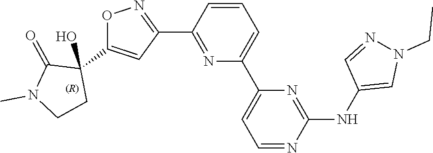

The title compound was made in a manner analogous to Example 31. The compound was purified by reverse-phase HPLC purification using basic media and column C8 to provide the title compound (58%). ¹H NMR (400 MHz, Methanol-d₄) δ 8.61-8.47 (m, 2H), 8.15 (dd, J=7.8, 1.2 Hz, 1H), 8.09 (t, J=7.8 Hz, 1H), 8.04 (d, J=0.8 Hz, 1H), 7.82 (d, J=5.1 Hz, 1H), 7.67 (s, 1H), 7.19 (s, 1H), 4.20 (q, J=7.3 Hz, 2H), 3.64-3.55 (m, 2H), 2.97 (s, 3H), 2.77 (ddd, J=13.5, 6.9, 5.0 Hz, 1H), 2.48-2.38 (m, 1H), 1.49 (t, J=7.3 Hz, 3H). Mass calcd. for C₂₂H₂₂N₈O₃ 446.2 m/z found 446.7 [M+H]⁺.

### Example 47: (R)-3-Hydroxy-1-methyl-3-(3-(6-(2-((1-methyl-1H-pyrazol-3-yl)amino)pyrimidin-4-yl)pyridin-2-yl)isoxazol-5-yl)pyrrolidin-2-one

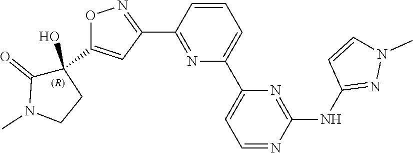

The title compound was made in a manner analogous to Example 31. The compound was purified by reverse-phase HPLC purification (Xtimate C18 column, 10 μm, 150×40 mm; 20-50% ACN/H₂O with 0.05% NH₃·H₂O) to provide the title compound (49%). ¹H NMR (400 MHz, Methanol-d₄) δ 8.58 (d, J=5.1 Hz, 1H), 8.53 (dd, J=7.7, 1.2 Hz, 1H), 8.14 (dd, J=7.8, 1.2 Hz, 1H), 8.07 (t, J=7.8 Hz, 1H), 7.90 (d, J=5.1 Hz, 1H), 7.51 (d, J=2.4 Hz, 1H), 7.19 (s, 1H), 6.74 (d, J=2.3 Hz, 1H), 3.83 (s, 3H), 3.63-3.55 (m, 2H), 2.97 (s, 3H), 2.77 (ddd, J=13.5, 6.9, 5.0 Hz, 1H), 2.42 (ddd, J=13.5, 8.0, 6.6 Hz, 1H). Mass calcd. for C₂₁H₂₀N₈O₃ 432.2 m/z found 432.7 [M+H]⁺.

### Example 48: (R)-3-Hydroxy-3-(3-(6-(2-((1-(2-hydroxyethyl)-3-methoxy-1H-pyrazol-4-yl)amino)pyrimidin-4-yl)pyridin-2-yl)isoxazol-5-yl)-1-methylpyrrolidin-2-one

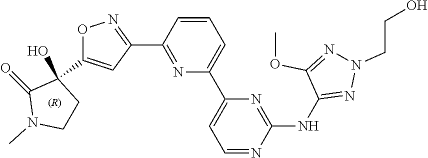

The title compound was made in a manner analogous to Example 31. The compound was purified by reverse-phase HPLC purification using acidic media and column C7 to provide the title compound (26%) as its TFA salt. ¹H NMR (400 MHz, Methanol-d₄) δ 8.56 (d, J=7.7 Hz, 1H), 8.47 (d, J=5.8 Hz, 1H), 8.21 (d, J=7.7 Hz, 1H), 8.12 (t, J=7.8 Hz, 1H), 7.97 (s, 1H), 7.86 (s, 1H), 7.19 (s, 1H), 4.10 (t, J=5.2 Hz, 2H), 3.95 (s, 3H), 3.90 (t, J=5.2 Hz, 2H), 3.62-3.55 (m, 2H), 2.97 (s, 3H), 2.81-2.72 (m, 1H), 2.49-2.38 (m, 1H). Mass calcd. for C₂₃H₂₄N₈O₅ 492.2 m/z found 493.3 [M+H]⁺. The TFA salt could be converted to the corresponding free base by passage through a carbonate cartridge.

### Example 49: (R)-3-Hydroxy-3-(3-(6-(2-((1-(2-hydroxy-2-methylpropyl)-3-methoxy-1H-pyrazol-4-yl)amino)pyrimidin-4-yl)pyridin-2-yl)isoxazol-5-yl)-1-methylpyrrolidin-2-one

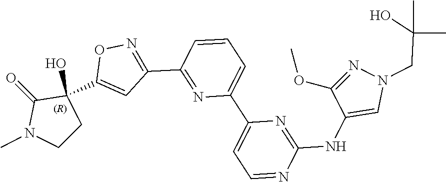

The title compound was made in a manner analogous to Example 31. The compound was purified by reverse-phase HPLC purification using acidic media and column C7 to provide the title compound (44%) as its TFA salt. ¹H NMR (400 MHz, Methanol-d₄) δ 8.55 (d, J=7.9 Hz, 1H), 8.49 (d, J=5.6 Hz, 1H), 8.20 (d, J=7.7 Hz, 1H), 8.11 (t, J=7.8 Hz, 1H), 7.99-7.85 (m, 2H), 7.19 (s, 1H), 3.95 (s, 3H), 3.63-3.53 (m, 2H), 2.97 (s, 3H), 2.82-2.71 (m, 1H), 2.42 (dt, J=14.1, 7.2 Hz, 1H), 1.24 (s, 6H). Mass calcd. for C₂₅H₂₈N₈O₅ 520.2 m/z found 521.3 [M+H]⁺. The TFA salt could be converted to the corresponding free base by passage through a carbonate cartridge.

### Example 50: (R)-3-Hydroxy-3-(3-(6-(2-((1-(3-hydroxycyclobutyl)-4-methyl-1H-pyrazol-3-yl)amino)pyrimidin-4-yl)pyridin-2-yl)isoxazol-5-yl)-1-methylpyrrolidin-2-one

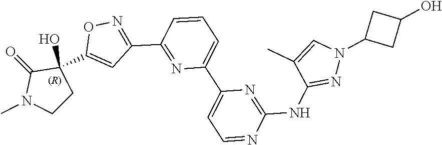

The title compound was made in a manner analogous to Example 31. The compound was purified by reverse-phase HPLC purification using acidic media and column C7 to provide the title compound (35%) as its TFA salt. ¹H NMR (400 MHz, Methanol-d₄) δ 8.56-8.44 (m, 2H), 8.22-8.18 (m, 1H), 8.11 (t, J=7.8 Hz, 1H), 7.97 (d, J=6.2 Hz, 2H), 7.19 (s, 1H), 5.01-4.91 (m, 1H), 4.58 (ddt, J=7.5, 6.5, 4.0 Hz, 1H), 3.58 (ddt, J=10.3, 6.7, 3.0 Hz, 2H), 2.97 (s, 3H), 2.84-2.70 (m, 4H), 2.55-2.38 (m, 4H), 2.25 (s, 3H). Mass calcd. for C₂₅H₂₆N₈O₄ 502.2 m/z found 503.0 [M+H]⁺. The TFA salt could be converted to the corresponding free base by passage through a carbonate cartridge.

### Example 51: (R)-3-Hydroxy-3-(3-(6-(2-(isoxazol-4-ylamino)pyrimidin-4-yl)pyridin-2-yl)isoxazol-5-yl)-1-methylpyrrolidin-2-one

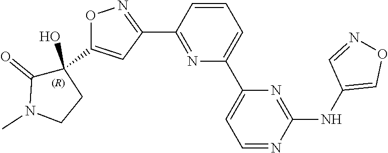

The title compound was made in a manner analogous to Example 31. The compound was purified by reverse-phase HPLC purification using basic media and column C8 to provide the title compound (7%). ¹H NMR (400 MHz, Methanol-d₄) δ 9.17 (s, 1H), 8.65 (d, J=5.1 Hz, 1H), 8.60 (s, 1H), 8.53 (dd, J=7.6, 1.3 Hz, 1H), 8.21-8.07 (m, 2H), 7.93 (d, J=5.1 Hz, 1H), 7.19 (s, 1H), 3.64-3.56 (m, 2H), 2.98 (s, 3H), 2.77 (ddd, J=13.6, 6.9, 5.0 Hz, 1H), 2.42 (ddd, J=13.5, 8.0, 6.7 Hz, 1H). Mass calcd. for C₂₀H₁₇N₇O₄ 419.1 m/z found 420.2 [M+H]⁺.

### Example 52: (R)-3-Hydroxy-1-methyl-3-(3-(6-(2-((1-methyl-1H-pyrazol-4-yl)amino)pyrimidin-4-yl)pyridin-2-yl)isoxazol-5-yl)pyrrolidin-2-one

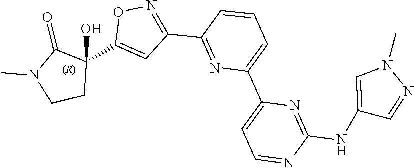

The title compound was made in a manner analogous to Example 31. The compound was purified by reverse-phase HPLC purification using basic media and column C2 to provide the title compound (64%).

The title compound was also prepared through the following method. A mixture of (R)-3-hydroxy-1-methyl-3-(3-(6-(2-(methylsulfonyl)pyrimidin-4-yl)pyridin-2-yl)isoxazol-5-yl)pyrrolidin-2-one (100 mg, 0.24 mmol, Intermediate 13), 4-amino-1-methylpyrazole (70 mg, 0.72 mmol) and TFA (0.037 mL, 0.48 mmol) in DMSO (1 mL) was heated at 145° C. for 1 h, then cooled to rt. The mixture was poured into a rapidly stirring mixture of 0.5 M aqueous K₂CO₃ (10 mL) and the resulting mixture was cooled to 0° C. and stirred at 0° C. for 30 min. The precipitate that formed was collected by filtration and washed with ice water (5 mL). The brown solid obtained was dried under air overnight to provide the title compound as a beige solid (78 mg, 75%). ¹H NMR (500 MHz, DMSO-d₆) δ 9.63 (s, 1H), 8.63 (d, J=5.0 Hz, 1H), 8.49 (d, J=7.7 Hz, 1H), 8.21 (t, J=7.8 Hz, 1H), 8.16 (dd, J=7.8, 1.2 Hz, 1H), 7.98 (s, 1H), 7.76 (d, J=5.0 Hz, 1H), 7.63-7.54 (m, 1H), 7.17 (s, 1H), 6.76 (s, 1H), 3.86 (s, 3H), 3.56-3.40 (m, 2H), 2.85 (s, 3H), 2.68-2.57 (m, 1H), 2.34-2.25 (m, 1H). MS (ESI⁺): m/z=433.4.

### Example 53: (R)-3-Hydroxy-1-methyl-3-(3-(6-(2-(pyrazolo[1,5-a]pyridin-3-ylamino)pyrimidin-4-yl)pyridin-2-yl)isoxazol-5-yl)pyrrolidin-2-one

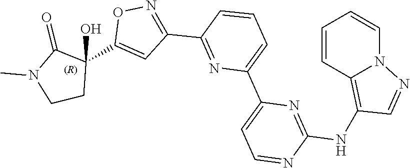

The title compound was made in a manner analogous to Example 31. The compound was purified by reverse-phase HPLC purification using basic media and column C8 to provide the title compound (32%). ¹H NMR (500 MHz, DMSO-d₆) δ 9.67 (s, 1H), 8.65 (d, J=5.0 Hz, 1H), 8.58 (dt, J=7.0, 1.1 Hz, 1H), 8.46 (s, 1H), 8.41 (s, 1H), 8.23-8.11 (m, 2H), 7.91-7.78 (m, 2H), 7.17 (s, 1H), 7.16-7.09 (m, 1H), 6.89-6.80 (m, 1H), 6.77 (s, 1H), 3.53-3.42 (m, 2H), 2.85 (s, 3H), 2.67-2.57 (m, 1H), 2.34-2.25 (m, 1H). MS (ESI⁺): m/z=469.5.

### Example 54: (R)-3-(3-(6-(2-((3-(2,2-Difluoroethoxy)-1-methyl-1H-pyrazol-4-yl)amino)pyrimidin-4-yl)pyridin-2-yl)isoxazol-5-yl)-3-hydroxy-1-methylpyrrolidin-2-one

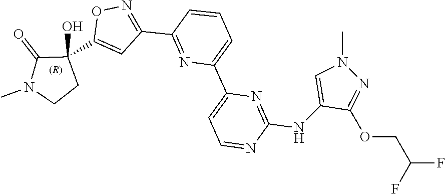

The title compound was made in a manner analogous to Example 31. The compound was purified by reverse-phase HPLC purification using basic media and column C8 to provide the title compound (35%). ¹H NMR (500 MHz, DMSO-d₆) δ 8.64 (s, 1H), 8.57 (d, J=5.0 Hz, 1H), 8.39 (s, 1H), 8.20-8.08 (m, 2H), 7.80 (s, 1H), 7.75 (d, J=5.0 Hz, 1H), 7.15 (s, 1H), 6.76 (s, 1H), 6.33 (tt, J=54.8, 3.8 Hz, 1H), 4.39 (td, J=14.7, 3.8 Hz, 2H), 3.73 (s, 3H), 3.57-3.39 (m, 2H), 2.85 (s, 3H), 2.70-2.53 (m, 1H), 2.34-2.18 (m, 1H). MS (ESI⁺): m/z=513.5.

### Example 55: (R)-3-(3-(6-(2-((3-Ethoxy-1-methyl-1H-pyrazol-4-yl)amino)pyrimidin-4-yl)pyridin-2-yl)isoxazol-5-yl)-3-hydroxy-1-methylpyrrolidin-2-one

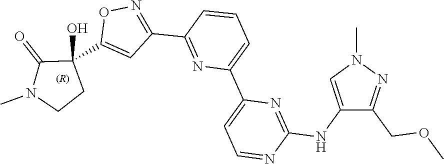

The title compound was made in a manner analogous to Example 31. The compound was purified by reverse-phase HPLC purification using basic media and column C8 to provide the title compound (25%). ¹H NMR (500 MHz, DMSO-d₆) δ 8.56 (d, J=5.0 Hz, 1H), 8.48 (s, 1H), 8.40 (s, 1H), 8.20-8.05 (m, 2H), 7.81-7.68 (m, 2H), 7.15 (s, 1H), 6.76 (s, 1H), 4.17 (q, J=7.0 Hz, 2H), 3.71 (s, 3H), 3.55-3.38 (m, 3H), 2.85 (s, 3H), 2.67-2.56 (m, 1H), 2.35-2.19 (m, 1H), 1.27 (t, J=7.0 Hz, 3H). MS (ESI⁺): m/z=477.5.

### Example 56: (R)-3-(3-(6-(2-((1-(2,2-Difluoroethyl)-1H-pyrazol-4-yl)amino)pyrimidin-4-yl)pyridin-2-yl)isoxazol-5-yl)-3-hydroxy-1-methylpyrrolidin-2-one

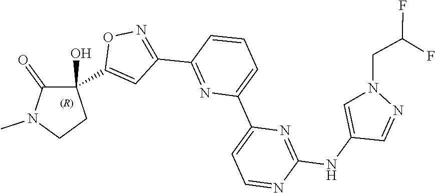

The title compound was made in a manner analogous to Example 31. The compound was purified by reverse-phase HPLC purification using basic media and column C8 to provide the title compound (35%). ¹H NMR (500 MHz, DMSO-d₆) δ 9.72 (s, 1H), 8.66 (d, J=5.0 Hz, 1H), 8.49 (d, J=7.5 Hz, 1H), 8.26-8.14 (m, 2H), 8.12 (s, 1H), 7.79 (d, J=5.0 Hz, 1H), 7.73-7.58 (m, 1H), 7.17 (s, 1H), 6.77 (s, 1H), 6.37 (tt, J=55.0, 3.8 Hz, 2H), 4.72-4.55 (m, 2H), 3.57-3.40 (m, 2H), 2.85 (s, 3H), 2.69-2.57 (m, 1H), 2.39-2.20 (m, 1H). MS (ESI⁺): m/z=483.4.

### Example 57: (R)-3-(3-(6-(2-((1-(2,2-Difluoroethyl)-3-methyl-1H-pyrazol-4-yl)amino)pyrimidin-4-yl)pyridin-2-yl)isoxazol-5-yl)-3-hydroxy-1-methylpyrrolidin-2-one

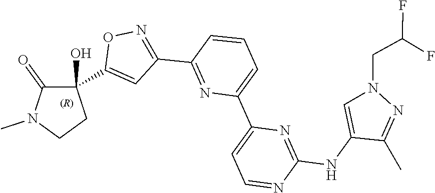

The title compound was made in a manner analogous to Example 31. The compound was purified by reverse-phase HPLC purification using basic media and column C8 to provide the title compound (30%). ¹H NMR (500 MHz, DMSO-d₆) δ 9.04 (s, 1H), 8.62 (d, J=5.0 Hz, 1H), 8.44 (d, J=6.6 Hz, 1H), 8.24-8.11 (m, 2H), 8.07 (s, 1H), 7.78 (d, J=5.0 Hz, 1H), 7.16 (s, 1H), 6.76 (s, 1H), 6.35 (tt, J=55.2, 3.9 Hz, 1H), 4.62-4.48 (m, 2H), 3.59-3.41 (m, 2H), 2.85 (s, 3H), 2.68-2.55 (m, 1H), 2.36-2.24 (m, 1H), 2.19 (s, 3H). MS (ESI⁺): m/z=497.4.

### Example 58: (R)-3-(3-(6-(2-((1-(2,2-Difluoroethyl)-3-methoxy-1H-pyrazol-4-yl)amino)pyrimidin-4-yl)pyridin-2-yl)isoxazol-5-yl)-3-hydroxy-1-methylpyrrolidin-2-one

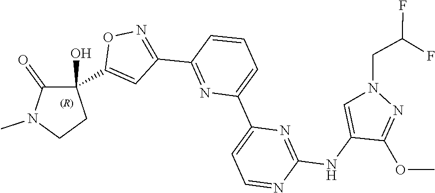

The title compound was made in a manner analogous to Example 31. The compound was purified by reverse-phase HPLC purification using basic media and column C8 to provide the title compound (20%). ¹H NMR (500 MHz, DMSO-d₆) δ 8.68 (s, 1H), 8.58 (d, J=5.0 Hz, 1H), 8.41 (s, 1H), 8.20-8.09 (m, 2H), 7.93 (s, 1H), 7.76 (d, J=5.0 Hz, 1H), 7.16 (s, 1H), 6.76 (s, 1H), 6.32 (tt, J=55.3, 3.9 Hz, 1H), 4.56-4.39 (m, 2H), 3.85 (s, 3H), 3.57-3.40 (m, 2H), 2.85 (s, 3H), 2.66-2.56 (m, 1H), 2.34-2.23 (m, 1H). MS (ESI⁺): m/z=513.5.

### Example 59: (R)-3-Hydroxy-1-methyl-3-(3-(6-(2-((1-methyl-3-(2,2,2-trifluoroethoxy)-1H-pyrazol-4-yl)amino)pyrimidin-4-yl)pyridin-2-yl)isoxazol-5-yl)pyrrolidin-2-one

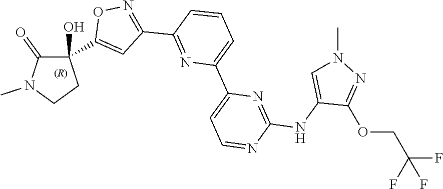

The title compound was made in a manner analogous to Example 31. The compound was purified by reverse-phase HPLC purification using basic media and column C8 to provide the title compound (24%). ¹H NMR (500 MHz, DMSO-d₆) δ 8.70 (s, 1H), 8.57 (d, J=5.0 Hz, 1H), 8.39 (s, 1H), 8.20-8.03 (m, 2H), 7.81 (s, 1H), 7.75 (d, J=5.0 Hz, 1H), 7.15 (s, 1H), 6.76 (s, 1H), 4.78 (q, J=9.0 Hz, 2H), 3.74 (s, 3H), 3.57-3.40 (m, 2H), 2.85 (s, 3H), 2.69-2.55 (m, 1H), 2.36-2.23 (m, 1H). MS (ESI⁺): m/z=531.4.

### Example 60: (R)-3-(3-(6-(2-((1-(2-Fluoroethyl)-1H-pyrazol-3-yl)amino)pyrimidin-4-yl)pyridin-2-yl)isoxazol-5-yl)-3-hydroxy-1-methylpyrrolidin-2-one

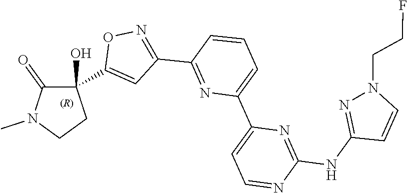

The title compound was made in a manner analogous to Example 31. The compound was purified by reverse-phase HPLC purification using basic media and column C8 to provide the title compound (33%). ¹H NMR (600 MHz, DMSO-d₆) δ 9.96 (s, 1H), 8.66 (d, J=5.0 Hz, 1H), 8.48 (dd, J=7.7, 1.1 Hz, 1H), 8.21 (t, J=7.7 Hz, 1H), 8.16 (dd, J=7.8, 1.1 Hz, 1H), 7.84 (d, J=5.0 Hz, 1H), 7.71 (d, J=2.3 Hz, 1H), 7.18 (s, 1H), 6.80-6.75 (m, 2H), 4.77 (dt, J=47.3, 4.7 Hz, 2H), 4.36 (dt, J=27.6, 4.8 Hz, 2H), 3.54-3.48 (m, 1H), 3.48-3.41 (m, 1H), 2.86 (s, 3H), 2.67-2.59 (m, 1H), 2.35-2.27 (m, 1H). MS (ESI⁺): m/z=465.3.

### Example 61: (R)-3-(3-(6-(2-((1-(2-Fluoroethyl)-1H-pyrazol-4-yl)amino)pyrimidin-4-yl)pyridin-2-yl)isoxazol-5-yl)-3-hydroxy-1-methylpyrrolidin-2-one

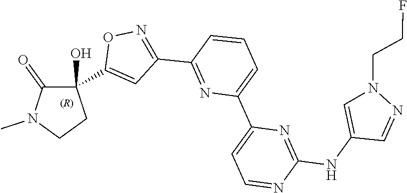

The title compound was made in a manner analogous to Example 31. The compound was purified by reverse-phase HPLC purification using basic media and column C8 to provide the title compound (54%). ¹H NMR (600 MHz, DMSO-d₆) δ 9.67 (s, 1H), 8.65 (d, J=4.7 Hz, 1H), 8.53-8.45 (m, 1H), 8.20 (t, J=7.7 Hz, 1H), 8.17 (dd, J=7.8, 1.2 Hz, 1H), 8.07 (s, 1H), 7.78 (d, J=5.0 Hz, 1H), 7.68 (s, 1H), 7.18 (s, 1H), 6.77 (s, 1H), 4.78 (dt, J=47.3, 4.8 Hz, 2H), 4.55-4.36 (m, 2H), 3.54-3.48 (m, 1H), 3.48-3.42 (m, 1H), 2.86 (s, 2H), 2.66-2.60 (m, 1H), 2.34-2.27 (m, 1H). MS (ESI⁺): m/z=465.4.

### Example 62: (R)-3-Hydroxy-1-methyl-3-(3-(6-(2-((1-(2-morpholinoethyl)-1H-pyrazol-4-yl)amino)pyrimidin-4-yl)pyridin-2-yl)isoxazol-5-yl)pyrrolidin-2-one

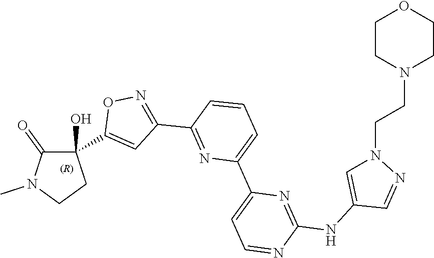

The title compound was made in a manner analogous to Example 31. The compound was purified by reverse-phase HPLC purification using acidic media and column C7 to provide the title compound as its TFA salt (23%). ¹H NMR (400 MHz, DMSO-d₆) δ 9.79 (s, 1H), 8.66 (d, J=5.0 Hz, 1H), 8.54-8.46 (m, 1H), 8.25-8.12 (m, 3H), 7.80 (d, J=5.0 Hz, 1H), 7.74 (s, 1H), 7.17 (s, 1H), 4.59 (t, J=6.4 Hz, 2H), 3.65 (t, J=6.4 Hz, 2H), 3.55-3.02 (m, 8H), 2.86 (s, 3H), 2.68-2.58 (m, 1H), 2.36-2.24 (m, 1H). MS (ESI⁺): m/z=533.4. The TFA salt could be converted to the corresponding free base by passage through a carbonate cartridge.

### Example 63: (R)-3-Hydroxy-1-methyl-3-(3-(6-(2-((5-methylisoxazol-4-yl)amino)pyrimidin-4-yl)pyridin-2-yl)isoxazol-5-yl)pyrrolidin-2-one

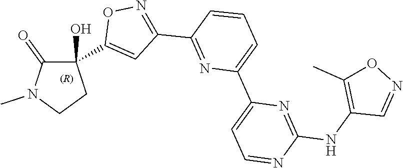

The title compound was made in a manner analogous to Example 31. The compound was purified by reverse-phase HPLC purification using basic media and column C8 to provide the title compound (5.6%). ¹H NMR (400 MHz, DMSO-d₆) δ 9.28 (s, 1H), 8.89 (s, 1H), 8.64 (d, J=5.1 Hz, 1H), 8.44-8.32 (m, 1H), 8.24-8.05 (m, 2H), 7.84 (d, J=5.0 Hz, 1H), 7.17 (s, 1H), 6.77 (s, 1H), 3.55-3.41 (m, 2H), 2.86 (s, 3H), 2.70-2.56 (m, 1H), 2.43 (s, 3H), 2.35-2.22 (m, 1H). MS (ESI⁺): m/z=434.3.

### Example 64: (R)-3-Hydroxy-3-(3-(6-(2-((1-(2-hydroxy-2-methylpropyl)-1H-pyrazol-4-yl)amino)pyrimidin-4-yl)pyridin-2-yl)isoxazol-5-yl)-1-methylpyrrolidin-2-one

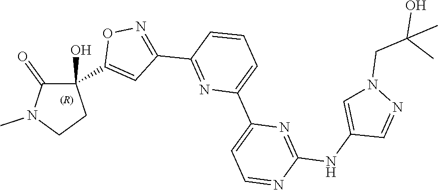

The title compound was made in a manner analogous to Example 31. The compound was purified by reverse-phase HPLC purification using acidic media and column C7 to provide the title compound as its TFA salt (17%). ¹H NMR (400 MHz, DMSO-d₆) δ 9.65 (s, 1H), 8.64 (d, J=5.0 Hz, 1H), 8.56-8.46 (m, 1H), 8.26-8.13 (m, 2H), 8.08 (s, 1H), 7.77 (d, J=5.0 Hz, 1H), 7.61 (s, 1H), 7.18 (s, 1H), 4.03 (s, 2H), 2.86 (s, 3H), 2.69-2.57 (m, 1H), 2.36-2.24 (m, 1H), 1.10 (s, 6H). MS (ESI⁺): m/z=491.4. The TFA salt could be converted to the corresponding free base by passage through a carbonate cartridge.

### Example 65: (R)-3-Hydroxy-1-methyl-3-(3-(6-(2-((1-methyl-1H-1,2,3-triazol-4-yl)amino)pyrimidin-4-yl)pyridin-2-yl)isoxazol-5-yl)pyrrolidin-2-one

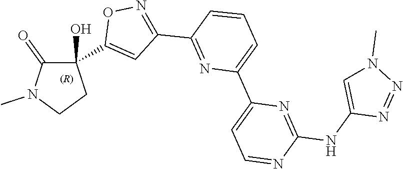

The title compound was made in a manner analogous to Example 31. The compound was purified by reverse-phase HPLC purification using acidic media and column C7 to provide the title compound as its TFA salt (9.4%). ¹H NMR (400 MHz, DMSO-d₆) δ 10.38 (s, 1H), 8.71 (d, J=5.1 Hz, 1H), 8.54 (dd, J=7.4, 1.5 Hz, 1H), 8.35-8.13 (m, 3H), 7.89 (d, J=5.0 Hz, 1H), 7.18 (s, 1H), 4.11 (s, 3H), 3.57-3.44 (m, 2H), 2.86 (s, 3H), 2.70-2.57 (m, 1H), 2.38-2.24 (m, 1H). MS (ESI⁺): m/z=434.2. The TFA salt could be converted to the corresponding free base by passage through a carbonate cartridge.

### Example 66: (R)-3-Hydroxy-1-methyl-3-(3-(6-(2-(oxetan-3-ylamino)pyrimidin-4-yl)pyridin-2-yl)isoxazol-5-yl)pyrrolidin-2-one

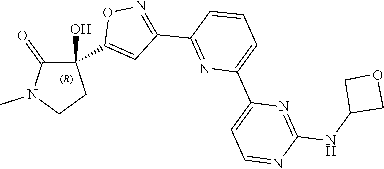

To a vial containing (R)-3-hydroxy-1-methyl-3-(3-(6-(2-(methylsulfonyl)pyrimidin-4-yl)pyridin-2-yl)isoxazol-5-yl)pyrrolidin-2-one (Intermediate 13, 50 mg, 0.12 mmol) was added oxetan-3-amine (18 mg, 0.24 mmol) followed by DMA (0.5 mL) and DIPEA (0.083 mL, 0.481 mmol). The reaction mixture was then heated at 150° C. for 3.5 h. The reaction mixture was then diluted with DMSO, filtered through a syringe-filter, and purified by preparative RP HPLC (basic conditions, column C8) to afford (R)-3-hydroxy-1-methyl-3-(3-(6-(2-(oxetan-3-ylamino)pyrimidin-4-yl)pyridin-2-yl)isoxazol-5-yl)pyrrolidin-2-one as a white solid (28%). LC-MS (ESI): Mass calcd. for C₂₀H₂₀N₆O₄, 408.15; m/z found, 409.4 [M+H]⁺. 1H NMR (500 MHz, DMSO-d₆) δ 8.53 (d, J=5.0 Hz, 1H), 8.45 (d, J=7.3 Hz, 1H), 8.22-8.01 (m, 3H), 7.69 (d, J=5.0 Hz, 1H), 7.14 (s, 1H), 6.75 (s, 1H), 5.05 (s, 1H), 4.92-4.75 (m, 2H), 4.60 (t, J=6.3 Hz, 2H), 3.54-3.38 (m, 2H), 2.85 (s, 3H), 2.69-2.57 (m, 1H), 2.35-2.18 (m, 1H).

### Example 67 and Example 68: (R)-3-Hydroxy-1-methyl-3-(3-(6-(2-(((R)-1-(1-methyl-1H-pyrazol-5-yl)ethyl)amino)pyrimidin-4-yl)pyridin-2-yl)isoxazol-5-yl)pyrrolidin-2-one and (R)-3-hydroxy-1-methyl-3-(3-(6-(2-(((S)-1-(1-methyl-1H-pyrazol-5-yl)ethyl)amino)pyrimidin-4-yl)pyridin-2-yl)isoxazol-5-yl)pyrrolidin-2-one

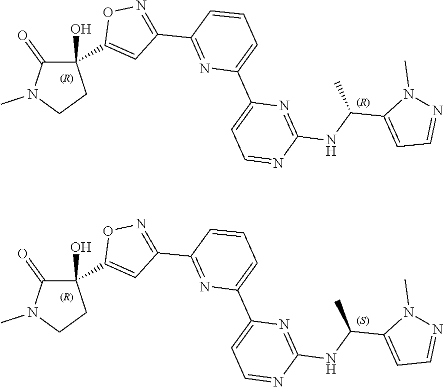

The title compounds were made in a manner analogous to Example 66. The reaction mixture was purified by reverse phase HPLC purification using basic media and column C8 to give (R)-3-hydroxy-1-methyl-3-(3-(6-(2-(((R,S)-1-(1-methyl-1H-pyrazol-5-yl)ethyl)amino)pyrimidin-4-yl)pyridin-2-yl)isoxazol-5-yl)pyrrolidin-2-one. The (R) and (S) diastereomers of (R)-3-hydroxy-1-methyl-3-(3-(6-(2-(((R,S)-1-(1-methyl-1H-pyrazol-5-yl)ethyl)amino)pyrimidin-4-yl)pyridin-2-yl)isoxazol-5-yl)pyrrolidin-2-one were separated by SFC (CHIRALPAK® IA, 5 μm, 250×21 mm; 45% methanol, 55% CO₂; flow rate 60 mL/min; monitoring at 220 nm) to give two products. The first eluting product was designated as diastereomer 1 of (R)-3-hydroxy-1-methyl-3-(3-(6-(2-((1-(1-methyl-1H-pyrazol-5-yl)ethyl)amino)pyrimidin-4-yl)pyridin-2-yl)isoxazol-5-yl)pyrrolidin-2-one (Example 67, 10%). ¹H NMR (500 MHz, DMSO-d₆) δ 8.45 (d, J=5.0 Hz, 1H), 8.36 (d, J=7.4 Hz, 1H), 8.15-8.01 (m, 2H), 7.73 (d, J=8.3 Hz, 1H), 7.58 (d, J=5.0 Hz, 1H), 7.21 (d, J=1.9 Hz, 1H), 7.07 (s, 1H), 6.68 (s, 1H), 6.17 (d, J=1.9 Hz, 1H), 5.38 (s, 1H), 3.77 (s, 3H), 3.50-3.31 (m, 2H), 2.78 (s, 3H), 2.60-2.51 (m, 1H), 2.26-2.15 (m, 1H), 1.46 (d, J=6.9 Hz, 3H). MS (ESI⁺): m/z=461.3. The second eluting product was designated as diastereomer 2 of (R)-3-hydroxy-1-methyl-3-(3-(6-(2-((-1-(1-methyl-1H-pyrazol-5-yl)ethyl)amino)pyrimidin-4-yl)pyridin-2-yl)isoxazol-5-yl)pyrrolidin-2-one (Example 68, 10%). ¹H NMR (500 MHz, DMSO-d₆) δ 8.45 (d, J=5.0 Hz, 1H), 8.36 (d, J=7.5 Hz, 1H), 8.16-7.94 (m, 2H), 7.73 (d, J=8.5 Hz, 1H), 7.58 (d, J=5.0 Hz, 1H), 7.21 (d, J=1.9 Hz, 1H), 7.07 (s, 1H), 6.68 (s, 1H), 6.17 (d, J=1.9 Hz, 1H), 5.39 (s, 1H), 3.77 (s, 3H), 3.46-3.32 (m, 2H), 2.78 (s, 3H), 2.58-2.50 (m, 1H), 2.25-2.16 (m, 1H), 1.46 (d, J=6.9 Hz, 3H). MS (ESI⁺): m/z=461.3.

### Example 69 and Example 70: (R)-3-Hydroxy-1-methyl-3-(3-(6-(2-(((R)-1-(1-methyl-1H-pyrazol-4-yl)ethyl)amino)pyrimidin-4-yl)pyridin-2-yl)isoxazol-5-yl)pyrrolidin-2-one and (R)-3-hydroxy-1-methyl-3-(3-(6-(2-(((S)-1-(1-methyl-1H-pyrazol-4-yl)ethyl)amino)pyrimidin-4-yl)pyridin-2-yl)isoxazol-5-yl)pyrrolidin-2-one

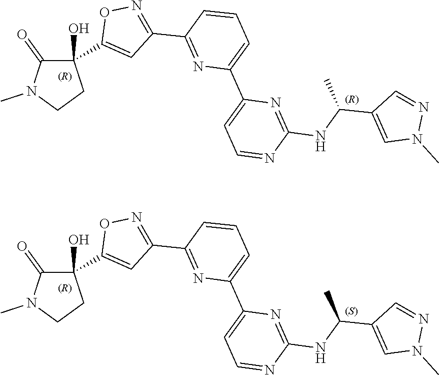

The title compounds were made in a manner analogous to Example 66. The reaction mixture was purified by reverse phase HPLC purification using basic media and column C8 to give (R)-3-hydroxy-1-methyl-3-(3-(6-(2-(((R,S)-1-(1-methyl-1H-pyrazol-4-yl)ethyl)amino)pyrimidin-4-yl)pyridin-2-yl)isoxazol-5-yl)pyrrolidin-2-one. The (R) and (S) diastereomers of (R)-3-hydroxy-1-methyl-3-(3-(6-(2-(((R,S)-1-(1-methyl-1H-pyrazol-4-yl)ethyl)amino)pyrimidin-4-yl)pyridin-2-yl)isoxazol-5-yl)pyrrolidin-2-one were separated by SFC (CHIRALPAK® IA, 5 μm, 250×21 mm; 45% methanol, 55% CO₂; flow rate 60 mL/min, monitoring at 220 nm) to give two products. The first eluting product was designated as diastereomer 1 of (R)-3-hydroxy-1-methyl-3-(3-(6-(2-((-1-(1-methyl-1H-pyrazol-4-yl)ethyl)amino)pyrimidin-4-yl)pyridin-2-yl)isoxazol-5-yl)pyrrolidin-2-one (22%, Example 69). ¹H NMR (500 MHz, DMSO-d₆) δ 8.49-8.28 (m, 2H), 8.11-8.02 (m, 2H), 7.58-7.51 (m, 2H), 7.44 (d, J=8.5 Hz, 1H), 7.33 (s, 1H), 7.07 (s, 1H), 6.69 (s, 1H), 5.19 (s, 1H), 3.70 (s, 3H), 3.46-3.32 (m, 2H), 2.78 (s, 3H), 2.58-2.49 (m, 1H), 2.26-2.15 (m, 1H), 1.42 (d, J=6.9 Hz, 3H). MS (ESI⁺): m/z=461.3. The second eluting product was designated as diastereomer 2 of (R)-3-hydroxy-1-methyl-3-(3-(6-(2-((-1-(1-methyl-1H-pyrazol-4-yl)ethyl)amino)pyrimidin-4-yl)pyridin-2-yl)isoxazol-5-yl)pyrrolidin-2-one (Example 70, 19%). ¹H NMR (500 MHz, DMSO-d₆) δ 8.50-8.30 (m, 2H), 8.12-8.01 (m, 2H), 7.61-7.50 (m, 2H), 7.44 (d, J=8.5 Hz, 1H), 7.33 (s, 1H), 7.07 (s, 1H), 6.69 (s, 1H), 5.19 (s, 1H), 3.70 (s, 3H), 3.47-3.32 (m, 2H), 2.78 (s, 3H), 2.63-2.48 (m, 1H), 2.27-2.17 (m, 1H). MS (ESI⁺): m/z=461.3.

### Example 71: (R)-3-Hydroxy-1-methyl-3-(3-(3-(2-((3,3,3-trifluoropropyl)amino)pyrimidin-4-yl)phenyl)isoxazol-5-yl)pyrrolidin-2-one

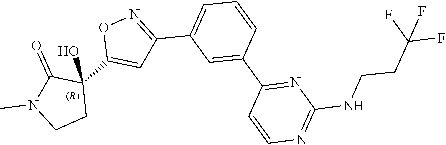

To a vial under an atmosphere of N₂, was added (R)-3-(3-(3-(2-chloropyrimidin-4-yl)phenyl)isoxazol-5-yl)-3-hydroxy-1-methylpyrrolidin-2-one (Intermediate 12, 50 mg, 0.135 mmol), 3,3,3-trifluoropropan-1-amine (60 mg, 0.539 mmol), N,N-diisopropylethylamine (0.117 mL, 0.674 mmol), and DMA (0.581 mL, 6.27 mmol). The reaction was heated in a microwave reactor at 150° C. for 1 hour and cooled to room temperature. The reaction mixture was diluted with DMSO, filtered, purified via RP HPLC (C18 (5 μm OBD) 50×100 mm, NH₄OH modifier, gradient 5-95% MeCN & H₂O) and concentrated under reduced pressure. The resulting residue was then subjected to further purification using acidic conditions with column C1 to yield (R)-3-hydroxy-1-methyl-3-(3-(3-(2-((3,3,3-trifluoropropyl)amino)pyrimidin-4-yl)phenyl)isoxazol-5-yl)pyrrolidin-2-one (46 mg, 72%). ¹H NMR (500 MHz, DMSO-d₆) δ 8.57 (d, J=2.0 Hz, 1H), 8.45 (d, J=5.2 Hz, 1H), 8.24 (d, J=7.6 Hz, 1H), 8.03 (d, J=7.6 Hz, 1H), 7.67 (t, J=7.8 Hz, 1H), 7.54 (s, 1H), 7.38 (d, J=5.4 Hz, 1H), 7.11 (s, 1H), 3.72-3.59 (m, 4H), 2.85 (s, 3H), 2.69-2.54 (m, 3H), 2.34-2.24 (m, 1H). MS (ESI⁺): m/z=448.

### Example 72: (R)-3-Hydroxy-1-methyl-3-(3-(3-(2-((1-methyl-1H-pyrazol-4-yl)amino)pyrimidin-4-yl)phenyl)isoxazol-5-yl)pyrrolidin-2-one

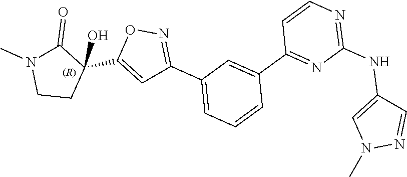

The title compound was made in a manner analogous to Example 71. The compound was purified via RP HPLC (C18 (5 μm OBD) 50×100 mm, NH₄OH modifier, gradient 5-95% MeCN & H₂O) and concentrated under reduced pressure. The resulting residue was then subjected to further purification using acidic conditions with column C1 to yield the titular compound (54%). ¹H NMR (500 MHz, DMSO-d₆) δ 9.59 (s, 1H), 8.64 (d, J=1.9 Hz, 1H), 8.54 (d, J=5.2 Hz, 1H), 8.27 (d, J=7.8 Hz, 1H), 8.11-8.01 (m, 1H), 7.97 (s, 1H), 7.70 (t, J=7.8 Hz, 1H), 7.56 (s, 1H), 7.43 (d, J=5.2 Hz, 1H), 7.13 (s, 1H), 3.84 (s, 3H), 2.85 (s, 3H), 2.58 (ddd, J=12.8, 7.6, 4.8 Hz, 1H), 2.29 (ddd, J=13.5, 8.0, 5.7 Hz, 1H). MS (ESI⁺): m/z=432.

### Example 73: (R)-3-Hydroxy-1-methyl-3-(3-(6-(2-((1-(methyl-d₃)-1H-pyrazol-4-yl)amino)pyrimidin-4-yl)pyridin-2-yl)isoxazol-5-yl)pyrrolidin-2-one

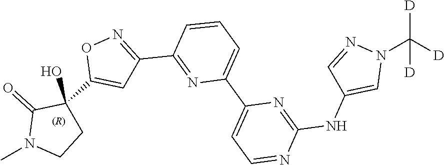

(R)-3-(3-(6-(2-((1H-Pyrazol-4-yl)amino)pyrimidin-4-yl)pyridin-2-yl)isoxazol-5-yl)-3-hydroxy-1-methylpyrrolidin-2-one (Example 40, 100 mg, 0.24 mmol) was dissolved in DMF (2.39 mL), and K₂CO₃ (198 mg, 1.43 mmol), and iodomethane-d₃ (0.032 mL, 0.48 mmol) were added. The reaction mixture was heated at 60° C. for 12 hours before an additional aliquot of iodomethane-d₃ (0.032 mL, 0.48 mmol) was added. The reaction was heated at 60° C. for an additional 6 hours before it was cooled to room temp, filtered, and subjected to HPLC purification (Xtimate C18 column, 10 μm, 150×40 mm, 20-50% ACN/TFA in H₂O/TFA) to provide the title compound as its TFA salt as a yellow solid (20 mg, TFA salt, 20%). ¹H NMR (400 MHz, Methanol-d₄) δ 8.59-8.51 (m, 2H), 8.19 (dd, J=7.8, 1.2 Hz, 1H), 8.12 (t, J=7.8 Hz, 1H), 8.01 (d, J=0.8 Hz, 1H), 7.92 (d, J=5.5 Hz, 1H), 7.68 (s, 1H), 7.19 (s, 1H), 3.64-3.55 (m, 2H), 2.77 (ddd, J=13.5, 7.0, 5.0 Hz, 1H), 2.42 (ddd, J=13.5, 7.9, 6.5 Hz, 1H). The TFA salt could be converted to the corresponding free base by passage through a carbonate cartridge.

### Example 74: (R)-3-(3-(6-(2-((1-(Ethyl-d₅)-1H-pyrazol-4-yl)amino)pyrimidin-4-yl)pyridin-2-yl)isoxazol-5-yl)-3-hydroxy-1-methylpyrrolidin-2-one

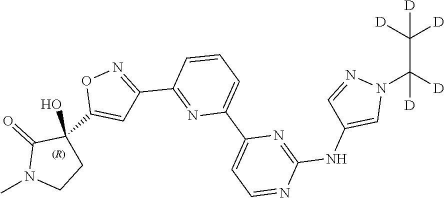

The title compound (40 mg, 37%) was prepared using conditions analogous to those described in Example 73, using iodoethane-d₅ (0.15 mL, 1.43 mmol) in place of iodomethane-d₃. ¹H NMR (400 MHz, Methanol-d₄) δ 8.56 (d, J=5.1 Hz, 1H), 8.52 (dd, J=7.7, 1.2 Hz, 1H), 8.15 (dd, J=7.9, 1.2 Hz, 1H), 8.09 (t, J=7.7 Hz, 1H), 8.03 (d, J=0.8 Hz, 1H), 7.82 (d, J=5.1 Hz, 1H), 7.67 (s, 1H), 7.19 (s, 1H), 3.63-3.55 (m, 2H), 2.98 (s, 3H), 2.77 (ddd, J=13.5, 6.9, 5.0 Hz, 1H), 2.42 (ddd, J=13.5, 8.0, 6.6 Hz, 1H). LC-MS (ESI): Mass calcd. for C₂₂H₁₇D₅N₈O₃ 451.2 m/z found 452.2 [M+H]⁺. The TFA salt could be converted to the corresponding free base by passage through a carbonate cartridge.

### Example 75: (R)-3-Hydroxy-1-methyl-3-(1-(6-(2-((1-methyl-1H-pyrazol-3-yl)amino)pyrimidin-4-yl)pyridin-2-yl)-1H-1,2,3-triazol-4-yl)pyrrolidin-2-one

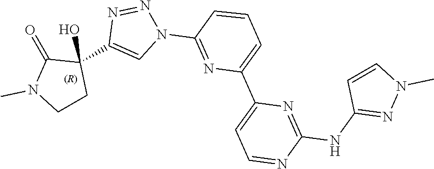

Step A. 4-(6-Bromopyridin-2-yl)-N-(1-methyl-1H-pyrazol-3-yl)pyrimidin-2-amine. To a 100 mL round-bottomed flask was added toluene (30 mL), N-(1-methyl-1H-pyrazol-3-yl)-4-(tributylstannyl)pyrimidin-2-amine (Intermediate 24, 2.5 g, 5.4 mmol), 2,6-dibromopyridine (1.5 g, 6.5 mmol), and triethylamine (1.5 mL, 11 mmol). The flask was evacuated and filled with N₂ three times. To this solution was added Pd(PPh₃)₄ (622 mg, 0.539 mmol) and the resulting mixture was heated for 16 h at 120° C. The reaction mixture was then diluted with H₂O (100 mL), extracted with EtOAc (100 mL×3) and the organic layer was washed with brine (100 mL), dried over anhydrous Na₂SO₄, filtered, and concentrated to dryness in vacuo to give a brown oil. The oil was subjected to silica gel chromatography (0-50% EtOAc/pet ether) to give 4-(6-bromopyridin-2-yl)-N-(1-methyl-1H-pyrazol-3-yl)pyrimidin-2-amine as a yellow solid (1 g, 46%).

Step B. (R)-3-Hydroxy-1-methyl-3-(1-(6-(2-((1-methyl-1H-pyrazol-3-yl)amino)pyrimidin-4-yl)pyridin-2-yl)-1H-1,2,3-triazol-4-yl)pyrrolidin-2-one. DMSO (1 mL), 4-(6-bromopyridin-2-yl)-N-(1-methyl-1H-pyrazol-3-yl)pyrimidin-2-amine (90 mg, 0.27 mmol), (R)-3-ethynyl-3-hydroxy-1-methylpyrrolidin-2-one (38 mg, 0.27 mmol), (1S,2S)-N¹,N²-dimethylcyclohexane-1,2-diamine (8 mg, 0.05 mmol), and CuI (10 mg, 0.054 mmol) were added to a flask. The mixture was sparged with Ar for 5 minutes and charged with NaN₃ (10 mg, 0.15 mmol), and sodium ascorbate (11 mg, 0.054 mmol) in H₂O (0.2 mL). The resultant mixture was stirred at r.t. for 16 h. After this time, the pH of mixture was adjusted to pH 9-10 with sat. aq. Na₂CO₃ (5 mL). This mixture was extracted with EtOAc (5 mL×3). The combined organic extracts were washed with brine (10 mL), dried over anhydrous Na₂SO₄, filtered, and concentrated in vacuo to give a yellow oil, which was subjected to HPLC (Phenomenex C18 column, 3 um, 75×30 mm; 20-50% (v/v) ACN/water (0.05% NH₃H₂O+10 mM NH₄HCO₃)) to give, after lyophillization, a yellow solid. The solid was further purified by SFC (DAICEL CHIRALPAK® OD-H column, 5 μm, 250 mm×30 mm; 50% (v/v) EtOH (0.1% NH₃H₂O)/CO₂) to give (R)-3-hydroxy-1-methyl-3-(1-(6-(2-((1-methyl-1H-pyrazol-3-yl)amino)pyrimidin-4-yl)pyridin-2-yl)-1H-1,2,3-triazol-4-yl)pyrrolidin-2-one (5.57 mg, 5%) as a yellow solid. LCMS (ESI): Mass calcd. for C₂₀H₂₀N₁₀O₂ 432.4 m/z, found 433.1 [M+1]⁺. ¹H NMR (400 MHz, DMSO-d₆): δ 9.95 (s, 1H), 8.95 (s, 1H), 8.67 (d, J=5.0 Hz, 1H), 8.46 (d, J=7.2 Hz, 1H), 8.39-8.31 (m, 1H), 8.29-8.21 (m, 1H), 7.82 (d, J=5.0 Hz, 1H), 7.67-7.55 (m, 1H), 6.82-6.64 (m, 1H), 6.34 (s, 1H), 3.78 (s, 3H), 3.53-3.46 (m, 2H), 2.83 (s, 3H), 2.81-2.73 (m, 1H), 2.31-2.23 (m, 1H).

### Example 76 and Example 77: (R)-3-Hydroxy-1-methyl-3-(1-(6-(2-((1-methyl-1H-pyrazol-4-yl)amino)pyrimidin-4-yl)pyridin-2-yl)-1H-pyrazol-4-yl)pyrrolidin-2-one and (S)-3-hydroxy-1-methyl-3-(1-(6-(2-((1-methyl-1H-pyrazol-4-yl)amino)pyrimidin-4-yl)pyridin-2-yl)-1H-pyrazol-4-yl)pyrrolidin-2-one (second enantiomer)

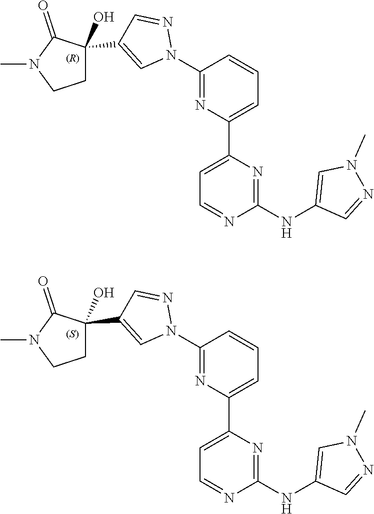

To a microwave vial, a mixture of Intermediate 22, (Enantiomer 1 of 3-hydroxy-1-methyl-3-(1-(6-(2-(methylsulfonyl)pyrimidin-4-yl)pyridin-2-yl)-1H-pyrazol-4-yl)pyrrolidin-2-one, 200 mg, 0.483 mmol), 1-methyl-1H-pyrazol-4-amine (480 mg, 4.9 mmol), TsOH (100 mg, 0.58 mmol) and 1,4-dioxane (6 mL) was heated at 115° C. for 16 h. The resulting mixture was cooled to rt, filtered through a syringe filter, diluted with DMSO and purified by RP HPLC (acidic conditions with column C7) to provide a residue. The residue was dissolved in EtOAc and washed with 1 M aqueous K₂CO₃. The organic layer was dried with MgSO₄, filtered, and concentrated to provide Example 76 (70 mg, 34%) as a yellow powder. LCMS (ESI): Mass calcd. for C₂₁H₂₁N₉O₂ 431.2 m/z found 432.1 [M+H]⁺. ¹H NMR (500 MHz, DMSO-d₆) δ 9.56 (s, 1H), 8.77-8.69 (m, 1H), 8.58 (d, J=5.0 Hz, 1H), 8.32-8.19 (m, 1H), 8.14 (t, J=7.9 Hz, 1H), 7.98 (dd, J=8.1, 0.9 Hz, 1H), 7.90 (s, 1H), 7.81 (d, J=0.8 Hz, 1H), 7.71 (d, J=5.0 Hz, 1H), 7.52 (s, 1H), 5.93 (s, 1H), 3.79 (s, 3H), 3.39-3.27 (m, 2H), 2.73 (s, 3H), 2.55-2.45 (m, 1H), 2.25-2.11 (m, 1H). Example 77 (70 mg, 34%) was made analogous to Example 76 using Enantiomer 2 of 3-hydroxy-1-methyl-3-(1-(6-(2-(methylsulfonyl)pyrimidin-4-yl)pyridin-2-yl)-1H-pyrazol-4-yl)pyrrolidin-2-one (Intermediate 23) in place of Intermediate 22. LCMS (ESI): Mass calcd. for C₂₁H₂₁N₉O₂ 431.2 m/z found 432.3 [M+H]⁺. ¹H NMR (500 MHz, DMSO-d₆) δ 9.56 (s, 1H), 8.73 (d, J=0.8 Hz, 1H), 8.58 (d, J=5.0 Hz, 1H), 8.25 (d, J=7.7 Hz, 1H), 8.14 (t, J=7.9 Hz, 1H), 7.98 (dd, J=8.1, 0.9 Hz, 1H), 7.90 (s, 1H), 7.81 (d, J=0.8 Hz, 1H), 7.71 (d, J=5.0 Hz, 1H), 7.52 (s, 1H), 5.93 (s, 1H), 3.79 (s, 3H), 3.36 (ddd, J=9.8, 8.1, 4.4 Hz, 1H), 3.33-3.27 (m, 1H), 2.73 (s, 3H), 2.51 (ddd, J=13.0, 7.4, 4.5 Hz, 1H), 2.19 (ddd, J=13.0, 8.1, 6.2 Hz, 1H).

### Example 78 and Example 79: (S)-3-Hydroxy-1-methyl-3-(2-(6-(2-((1-methyl-1H-pyrazol-3-yl)amino)pyrimidin-4-yl)pyridin-2-yl)thiazol-5-yl)pyrrolidin-2-one and (R)-3-hydroxy-1-methyl-3-(2-(6-(2-((1-methyl-1H-pyrazol-3-yl)amino)pyrimidin-4-yl)pyridin-2-yl)thiazol-5-yl)pyrrolidin-2-one

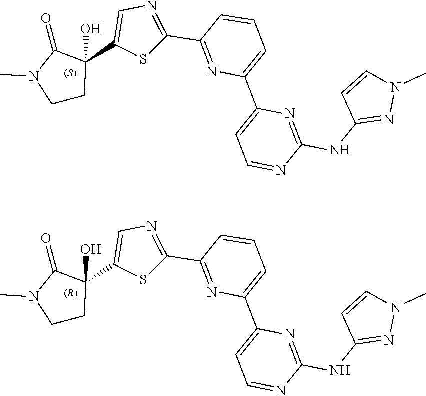

3-(2-(6-Chloropyridin-2-yl)thiazol-5-yl)-3-hydroxy-1-methylpyrrolidin-2-one (Intermediate 25, 210 mg, 0.68 mmol) and 1,4-dioxane (10 mL) were added to a nitrogen-purged 100 mL flask, and charged with N-(1-methyl-1H-pyrazol-3-yl)-4-(tributylstannyl)pyrimidin-2-amine (Intermediate 24, 450 mg, 0.97 mmol) and Pd(P(Cy)₃)₂Cl₂ (950 mg, 1.3 mmol). The resultant mixture was purged with Ar 3 times and then heated at 105° C. for 16 h. After this time, the mixture was cooled to r.t., treated with saturated KF (80 mL) and stirred for 1 h. This mixture was extracted with EtOAc (80 mL×3) and the combined organic extracts washed with brine, dried over anhydrous Na₂SO₄, filtered, and concentrated to dryness under reduced pressure to give a yellow oil. The oil was subjected to HPLC (Boston Prime C18 150×30 mm, 5 μm, 25-55% ACN/water with 0.05% NH₃H₂O+10 mM NH₄HCO₃) to give (R,S)-3-hydroxy-1-methyl-3-(2-(6-(2-((1-methyl-1H-pyrazol-3-yl)amino)pyrimidin-4-yl)pyridin-2-yl)thiazol-5-yl)pyrrolidin-2-one as a yellow solid (80 mg, 25%). LCMS (ESI): Mass calcd. For C₂₁H₂₀N₈O₂S 448.14 m/z, found 449.1 [M+1]⁺.

The (R) and (S) eantiomers of (R,S)-3-hydroxy-1-methyl-3-(2-(6-(2-((1-methyl-1H-pyrazol-3-yl)amino)pyrimidin-4-yl)pyridin-2-yl)thiazol-5-yl)pyrrolidin-2-one were separated by SFC (DAICEL CHIRALPAK AS column, 10 μm, 250×30 mm; 40% (v/v) EtOH (containing 0.1% of 25% aq. NH₃)/CO₂) to give two products. The first eluting peak was designated enantiomer 1 (Example 78, yellow solid, 38 mg, 46%). LC-MS (ESI): Mass calcd. for C₂₁H₂₀N₈O₂S 448.1 m/z found 449.2 [M+H]⁺. ¹H NMR (400 MHz, DMSO-d₆) δ 9.93 (s, 1H), 8.66 (d, J=4.8 Hz, 1H), 8.46-8.38 (m, 1H), 8.28-8.13 (m, 2H), 7.92 (s, 1H), 7.75 (d, J=4.8 Hz, 1H), 7.62 (d, J=2.0 Hz, 1H), 6.73 (d, J=2.0 Hz, 1H), 6.60 (s, 1H), 3.78 (s, 3H), 3.51-3.38 (m, 2H), 2.82 (s, 3H), 2.66-2.59 (m, 1H), 2.41-2.34 (m, 1H). The second eluting peak was designated enantiomer 2 (Example 79, 41 mg, 51%). LC-MS (ESI): Mass calcd. for C₂₁H₂₀N₈O₂S 448.1 m/z found 449.2 [M+H]⁺. ¹H NMR (400 MHz, DMSO-d₆) δ 9.93 (s, 1H), 8.66 (d, J=5.2 Hz, 1H), 8.45-8.38 (m, 1H), 8.26-8.15 (m, 2H), 7.92 (s, 1H), 7.75 (d, J=5.2 Hz, 1H), 7.62 (d, J=2.0 Hz, 1H), 6.73 (d, J=2.0 Hz, 1H), 6.60 (br s, 1H), 3.78 (s, 3H), 3.50-3.40 (m, 2H), 2.82 (s, 3H), 2.66-2.59 (m, 1H), 2.41-2.34 (m, 1H).

### Example 80 and Example 81: (S)-3-Hydroxy-1-methyl-3-(2-(6-(2-((1-methyl-1H-pyrazol-3-yl)amino)pyrimidin-4-yl)pyridin-2-yl)thiazol-4-yl)pyrrolidin-2-one and (R)-3-hydroxy-1-methyl-3-(2-(6-(2-((1-methyl-1H-pyrazol-3-yl)amino)pyrimidin-4-yl)pyridin-2-yl)thiazol-4-yl)pyrrolidin-2-one

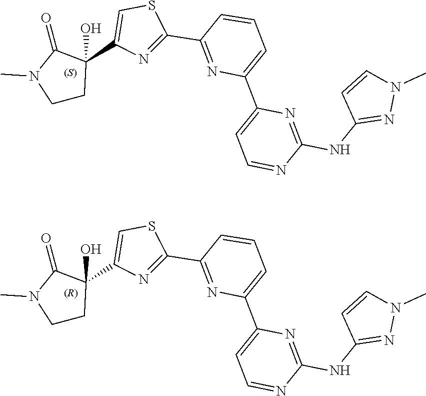

3-(2-(6-Chloropyridin-2-yl)thiazol-4-yl)-3-hydroxy-1-methylpyrrolidin-2-one (Intermediate 26, 250 mg, 0.81 mmol), and toluene (10 mL) were added to a nitrogen-purged 100 mL flask, and charged with N-(1-methyl-1H-pyrazol-3-yl)-4-(tributylstannyl)pyrimidin-2-amine (Intermediate 24, 750 mg, 1.6 mmol), TEA (0.25 mL, 1.8 mmol) and Pd(PPh₃)₄ (100 mg, 0.09 mmol). The resultant mixture was purged with Ar 3 times and then heated at 120° C. for 10 h. After this time, the mixture was cooled to r.t., quenched with saturated aqueous KF (20 mL), and stirred for 1 h. This mixture was extracted with EtOAc (20 mL×3) and the combined organic extracts washed with brine, dried over anhydrous Na₂SO₄, filtered, and concentrated to dryness under reduced pressure to give a black oil. This oil was then subjected to silica gel chromatography (0-100% EtOAc/pet ether, then 15% MeOH/DCM) to give (R,S)-3-hydroxy-1-methyl-3-(2-(6-(2-((1-methyl-1H-pyrazol-3-yl)amino)pyrimidin-4-yl)pyridin-2-yl)thiazol-4-yl)pyrrolidin-2-one as a yellow solid (50 mg). LCMS (ESI): Mass calcd. for C₂₁H₂₀N₈O₂S 448.14 m/z, found 449.1 [M+1]⁺.

The (R) and (S) eantiomers of (R,S)-3-hydroxy-1-methyl-3-(2-(6-(2-((1-methyl-1H-pyrazol-3-yl)amino)pyrimidin-4-yl)pyridin-2-yl)thiazol-4-yl)pyrrolidin-2-one were separated by SFC (DAICEL CHIRALPAK® AS column, 10 μm, 250×30 mm; 45% (v/v) EtOH (containing 0.1% of 25% aq. NH₃)/CO₂) to give two products. The first eluting product was designated as enantiomer 1 (3.7 mg, yellow solid, Example 80). LC-MS (ESI): Mass calcd. for C₂₁H₂₀N₈O₂S 448.14 m/z found 449.2 [M+H]⁺. ¹H NMR (400 MHz, DMSO-d₆) δ 9.93 (s, 1H), 8.69-8.63 (m, 1H), 8.45-8.39 (m, 1H), 8.23-8.17 (m, 2H), 7.77-7.69 (m, 2H), 7.62 (d, J=2.0 Hz, 1H), 6.73 (d, J=2.0 Hz, 1H), 6.33 (br s, 1H), 3.78 (s, 3H), 3.64-3.54 (m, 1H), 3.52-3.44 (m, 1H), 2.85 (s, 3H), 2.70-2.59 (m, 1H), 2.28-2.18 (m, 1H). The second eluting peak was designated as enantiomer 2 (Example 81, yellow solid, 5.0 mg). LC-MS (ESI): Mass calcd. for C₂₁H₂₀N₈O₂S 448.1 m/z found 449.1 [M+H]⁺. ¹H NMR (400 MHz, DMSO-d₆) δ 9.93 (s, 1H), 8.70-8.64 (m, 1H), 8.45-8.40 (m, 1H), 8.22-8.17 (m, 2H), 7.76-7.70 (m, 2H), 7.62 (d, J=2.0 Hz, 1H), 6.73 (d, J=2.0 Hz, 1H), 6.34 (br s, 1H), 3.78 (s, 3H), 3.64-3.54 (m, 1H), 3.53-3.44 (m, 1H), 2.85 (s, 3H), 2.70-2.60 (m, 1H), 2.29-2.18 (m, 1H).

### Example 82 and Example 83: (S)-3-Hydroxy-1-methyl-3-(5-(6-(2-((1-methyl-1H-pyrazol-3-yl)amino)pyrimidin-4-yl)pyridin-2-yl)thiazol-2-yl)pyrrolidin-2-one and (R)-3-hydroxy-1-methyl-3-(5-(6-(2-((1-methyl-1H-pyrazol-3-yl)amino)pyrimidin-4-yl)pyridin-2-yl)thiazol-2-yl)pyrrolidin-2-one

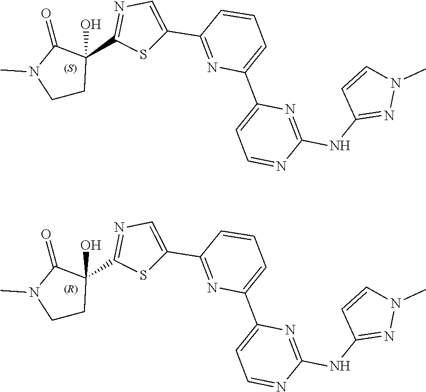

Step A. (R, S)-3-Hydroxy-1-methyl-3-(5-(6-(2-((1-methyl-1H-pyrazol-3-yl)amino)pyrimidin-4-yl)pyridin-2-yl)thiazol-2-yl)pyrrolidin-2-one. 3-(5-(6-Chloropyridin-2-yl)thiazol-2-yl)-3-hydroxy-1-methylpyrrolidin-2-one (Intermediate 27, 80 mg, 0.26 mmol), N-(1-methyl-1H-pyrazol-3-yl)-4-(tributylstannyl)pyrimidin-2-amine (Intermediate 24, 140 mg, 0.302 mmol) and 1,4-dioxane (1 mL) were added into a 8 mL microwave tube, which was subsequently evacuated and refilled with argon (×3), then treated with Pd(dppf)Cl₂ (38 mg, 0.052 mmol). The resulting mixture stirred for 1 h at 100° C. The reaction vessel was allowed to gradually cool to room temperature and the resulting mixture was stirred for 2 h after it was treated with saturated KF (10 mL) over the course of 1 min. The mixture was then extracted with ethyl acetate (15 mL×3), and the combined extracts washed with brine (20 mL), dried over anhydrous Na₂SO₄, filtered, and concentrated to dryness in vacuo to give a black solid. The solid was then subjected to HPLC (Boston Prime C18 column, 5 μm, 150×30 mm; 30-60% (v/v) ACN/H₂O with 0.05% NH₃·H₂O+10 mM NH₄HCO₃) to afford (R,S)-3-hydroxy-1-methyl-3-(5-(6-(2-((1-methyl-1H-pyrazol-3-yl)amino)pyrimidin-4-yl)pyridin-2-yl)thiazol-2-yl)pyrrolidin-2-one (22 mg, 19%) as a yellow solid. LCMS (ESI): Mass calcd. for C₂₁H₂₀N₈O₂S 448.1 m/z, found 448.9 [M+H]⁺.

Step B. The (R) and (S) enantiomers of (R,S)-3-hydroxy-1-methyl-3-(5-(6-(2-((1-methyl-1H-pyrazol-3-yl)amino)pyrimidin-4-yl)pyridin-2-yl)thiazol-2-yl)pyrrolidin-2-one (36 mg, 0.08 mmol) were separated by SFC (DAICEL CHIRALPAK AS column, 10 μm, 250×30 mm; 50% (v/v) EtOH (containing 0.1% of 25% aq. NH₃)/CO₂) to give two products. The first eluting peak was designated as enantiomer 1 (Example 82, white solid, 2 mg, 5%). LC-MS (ESI): Mass calcd. for C₂₁H₂₀N₈O₂S 448.1 m/z found 449.1 [M+H]⁺. ¹H NMR (400 MHz, DMSO-d₆): δ 9.88 (s, 1H), 8.64 (d, J=5.0 Hz, 1H), 8.48 (s, 1H), 8.34-8.25 (m, 1H), 8.16-8.06 (m, 2H), 7.72 (d, J=5.0 Hz, 1H), 7.62 (s, 1H), 6.96 (s, 1H), 6.72 (s, 1H), 3.78 (s, 3H), 3.54-3.44 (m, 2H), 2.83 (s, 3H), 2.78-2.71 (m, 1H), 2.32-2.24 (m, 1H). The second eluting peak was designated as enantiomer 2 (Example 83, yellow solid, 10 mg, 29%). LC-MS (ESI): Mass calcd. for C₂₁H₂₀N₈O₂S 448.1 m/z found 449.1 [M+H]⁺. ¹H NMR (400 MHz, DMSO-d₆): δ 9.89 (s, 1H), 8.63 (d, J=5.0 Hz, 1H), 8.48 (s, 1H), 8.32-8.19 (m, 1H), 8.17-8.07 (m, 2H), 7.71 (d, J=5.0 Hz, 1H), 7.61 (d, J=2.1 Hz, 1H), 6.96 (s, 1H), 6.72 (d, J=2.1 Hz, 1H), 3.77 (s, 3H), 3.56-3.42 (m, 2H), 2.83 (s, 3H), 2.78-2.70 (m, 1H), 2.30-2.23 (m, 1H).

### Example 84 and Example 85: (S)-3-Hydroxy-1-methyl-3-(5-(6-(2-((1-methyl-1H-pyrazol-3-yl)amino)pyrimidin-4-yl)pyridin-2-yl)-1H-pyrazol-3-yl)pyrrolidin-2-one and (R)-3-hydroxy-1-methyl-3-(5-(6-(2-((1-methyl-1H-pyrazol-3-yl)amino)pyrimidin-4-yl)pyridin-2-yl)-1H-pyrazol-3-yl)pyrrolidin-2-one

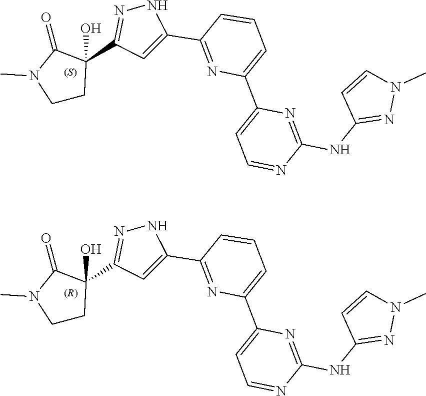

Step A. 3-Hydroxy-1-methyl-3-(3-(6-(2-((1-methyl-1H-pyrazol-3-yl)amino)pyrimidin-4-yl)pyridin-2-yl)-1-((2-(trimethylsilyl)ethoxy)methyl)-1H-pyrazol-5-yl)pyrrolidin-2-one. Toluene (2 mL), 3-(3-bromo-1-((2-(trimethylsilyl)ethoxy)methyl)-1H-pyrazol-5-yl)-3-hydroxy-1-methylpyrrolidin-2-one (Intermediate 29, 320 mg, 0.82 mmol), N-(1-methyl-1H-pyrazol-3-yl)-4-(6-(tributylstannyl)pyridin-2-yl)pyrimidin-2-amine (Intermediate 28, 490 mg, 0.91 mmol), and TEA (228 μL, 1.64 mmol) were added to a 10 mL flask and charged with Pd(PPh₃)₄ (95 mg, 0.082 mmol). The mixture was evacuated and refilled with N₂ for three times, and heated at 110° C. for 16 h. After this time, the mixture was cooled to r.t., diluted with H₂O (10 mL), and extracted with EtOAc (10 mL×3). The combined organic extracts were washed with brine (10 mL), dried over anhydrous Na₂SO₄, filtered, and concentrated to dryness in vacuo to give a brown oil. The oil was then was subjected to silica gel chromatography (0-100% EtOAc/pet ether) to give 3-hydroxy-1-methyl-3-(5-(6-(2-((1-methyl-1H-pyrazol-3-yl)amino)pyrimidin-4-yl)pyridin-2-yl)-1-((2-(trimethylsilyl)ethoxy)methyl)-1H-pyrazol-3-yl)pyrrolidin-2-one (300 mg, 64%) as a yellow solid. LCMS (ESI): Mass calcd. for C₂₇H₃₅N₉O₃Si 561.3 m/z, found 562.4 [M+H]⁺.

Step B. (R, S)-3-Hydroxy-1-methyl-3-(5-(6-(2-((1-methyl-1H-pyrazol-3-yl)amino)pyrimidin-4-yl)pyridin-2-yl)-1H-pyrazol-3-yl)pyrrolidin-2-one. Dichloromethane (2 mL), 3-hydroxy-1-methyl-3-(5-(6-(2-((1-methyl-1H-pyrazol-3-yl)amino)pyrimidin-4-yl)pyridin-2-yl)-1-((2-(trimethylsilyl)ethoxy)methyl)-1H-pyrazol-3-yl)pyrrolidin-2-one (280 mg, 0.5 mmol), and TFA (384 μL, 4.99 mmol) were added to an 8 mL flask. The mixture was stirred at r.t. for 16 h. After this time, the mixture was concentrated to dryness in vacuo to give a red oil, which was then subjected to HPLC (Boston Prime C18 column, 5 μm, 150×30 mm; 20-50% (v/v) MeCN and water (0.05% NH₃H₂O+10 mM NH₄HCO₃)) to give, after lyophillization, (R,S)-3-hydroxy-1-methyl-3-(5-(6-(2-((1-methyl-1H-pyrazol-3-yl)amino)pyrimidin-4-yl)pyridin-2-yl)-1H-pyrazol-3-yl)pyrrolidin-2-one (80 mg, 37%) as a yellow solid. LCMS (ESI): Mass calcd. for C₂₁H₂₁N₉O₂ 431.2 m/z, found 432.2 [M+1]⁺.

Step C. The (R) and (S) enantiomers of (R,S)-3-hydroxy-1-methyl-3-(5-(6-(2-((1-methyl-1H-pyrazol-3-yl)amino)pyrimidin-4-yl)pyridin-2-yl)-1H-pyrazol-3-yl)pyrrolidin-2-one (80 mg, 0.19 mmol) were separated by SFC (DAICEL CHIRALCEL OJ column, 10 μm, 250×30 mm; 40% (v/v) EtOH (containing 0.1% aq. NH₃)/CO₂) to afford two products. The first eluting peak was designated as enantiomer 1 (white solid, 20 mg, 24%, Example 84). LC-MS (ESI): Mass calcd. for C₂₁H₂₁N₉O₂ 431.5 m/z found 432.2 [M+H]⁺. ¹H NMR (400 MHz, DMSO-d₆): δ 13.68-12.95 (m, 1H), 9.85 (s, 1H), 8.72-8.56 (m, 1H), 8.39-8.22 (m, 1H), 8.18-7.80 (m, 3H), 7.69-7.57 (m, 1H), 7.12-6.84 (m, 1H), 6.79-6.68 (m, 1H), 6.41-5.87 (m, 1H), 3.77 (s, 3H), 3.46-3.41 (m, 2H), 2.87-2.70 (m, 4H), 2.28-2.06 (m, 1H). The second eluting peak was designated as enantiomer 2 (white solid, 15 mg, 18%, Example 85). LC-MS (ESI): Mass calcd. for C₂₁H₂₁N₉O₂ 431.45 m/z found 432.2 [M+H]⁺. ¹H NMR (400 MHz, DMSO-d₆): δ 13.62-12.95 (m, 1H), 9.85 (s, 1H), 8.69-8.52 (m, 1H), 8.37-8.21 (m, 1H), 8.18-8.01 (m, 2H), 8.00-7.78 (m, 1H), 7.65-7.58 (m, 1H), 7.09-6.87 (m, 1H), 6.72 (s, 1H), 6.38-5.92 (m, 1H), 3.77 (s, 3H), 3.42-3.39 (m, 2H), 2.88-2.70 (m, 4H), 2.31-2.12 (m, 1H).

### Example 86: (R)-3-(3-(6-(2-((5-Fluoropyridin-3-yl)amino)pyrimidin-4-yl)pyridin-2-yl)isoxazol-5-yl)-3-hydroxy-1-methylpyrrolidin-2-one

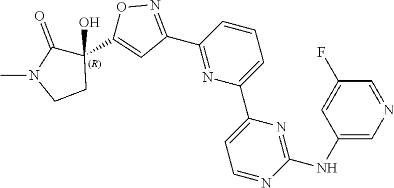

(R)-3-(3-(6-(2-Chloropyrimidin-4-yl)pyridin-2-yl)isoxazol-5-yl)-3-hydroxy-1-methylpyrrolidin-2-one (Intermediate 31, 55 mg, 0.15 mmol), cesium carbonate (193 mg, 0.592 mmol), Xantphos Pd G3 (15 mg, 0.015 mmol), 1,4-dioxane (1.5 mL) and 3-fluoro-5-aminopyridine (33 mg, 0.3 mmol) were heated at 100° C. for 16 h. The mixture was cooled to rt, diluted with ethyl acetate, filtered through diatomaceous earth, and concentrated. The residue was purified by RP HPLC (TFA modifier) to give (R)-3-(3-(6-(2-((5-fluoropyridin-3-yl)amino)pyrimidin-4-yl)pyridin-2-yl)isoxazol-5-yl)-3-hydroxy-1-methylpyrrolidin-2-one as a white solid (6.8 mg, 8%). ¹H NMR (500 MHz, DMSO-d₆) δ 10.06 (s, 1H), 8.74 (d, J=5.0 Hz, 1H), 8.68-8.64 (m, 1H), 8.48-8.43 (m, 1H), 8.43-8.36 (m, 1H), 8.28-8.14 (m, 2H), 7.94 (d, J=5.1 Hz, 1H), 7.21 (dd, J=8.9, 3.3 Hz, 1H), 7.19 (s, 1H), 3.55-3.43 (m, 2H), 2.86 (s, 3H), 2.69-2.58 (m, 1H), 2.34-2.24 (m, 1H). MS (ESI⁺): m/z=447.3.

### Example 87 and Example 88: (S)-3-Hydroxy-1-methyl-3-(1-(6-(2-((1-methyl-1H-pyrazol-3-yl)amino)pyrimidin-4-yl)pyridin-2-yl)-1H-imidazol-4-yl)pyrrolidin-2-one and (R)-3-hydroxy-1-methyl-3-(1-(6-(2-((1-methyl-1H-pyrazol-3-yl)amino)pyrimidin-4-yl)pyridin-2-yl)-1H-imidazol-4-ylpyrrolidin-2-one

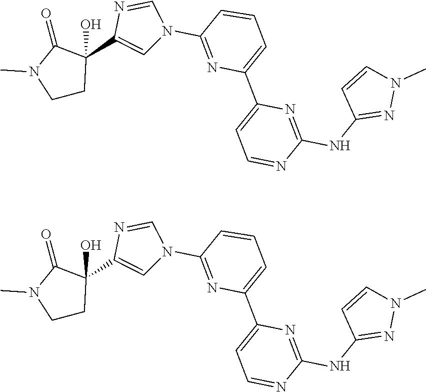

Step A. (R, S)-3-Hydroxy-1-methyl-3-(1-(6-(2-((1-methyl-1H-pyrazol-3-yl)amino)pyrimidin-4-yl)pyridin-2-yl)-1H-imidazol-4-yl)pyrrolidin-2-one. A mixture of 3-(1-(6-chloropyridin-2-yl)-1H-imidazol-4-yl)-3-hydroxy-1-methylpyrrolidin-2-one (Intermediate 30, 81 mg, 0.28 mmol), N-(1-methyl-1H-pyrazol-3-yl)-4-(trimethylstannyl)pyrimidin-2-amine (Intermediate 10, 140 mg, 0.415 mmol), [1,1′-bis(diphenylphosphino)ferrocene]dichloropalladium(ii) (41 mg, 0.055 mmol), cuprous iodide (21 mg, 0.11 mmol) and DMF (3.2) was heated at 130° C. for 1 h. The reaction mixture was cooled to rt, diluted with DMSO and filtered through a metal-scavenging thiol cartridge. The resulting solution was combined with a solution from another batch on % the scale and purified by RP HPLC (NH₄₀H modifier) to afford (R,S)-3-hydroxy-1-methyl-3-(1-(6-(2-((1-methyl-1H-pyrazol-3-yl)amino)pyrimidin-4-yl)pyridin-2-yl)-1H-imidazol-4-yl)pyrrolidin-2-one (73 mg, 61%) as a yellow solid.

Step B. The (R) and (S) enantiomers of (R,S)-3-hydroxy-1-methyl-3-(1-(6-(2-((1-methyl-1H-pyrazol-3-yl)amino)pyrimidin-4-yl)pyridin-2-yl)-1H-imidazol-4-yl)pyrrolidin-2-one (71 mg, 0.17 mmol) were separated by SFC (Chiralcel OD-H column, 5 μm, 250×21 mm 40% (v/v) MeOH/CO₂)) to afford two products. The first eluting peak was designated as enantiomer 1 (white solid, 10.6 mg, 9%) (Example 87). LCMS (ESI⁺): m/z=431.3. ¹H NMR (600 MHz, DMSO-d₆) δ 9.80 (s, 1H), 8.58 (d, J=5.0 Hz, 1H), 8.56-8.51 (m, 1H), 8.27-8.19 (m, 1H), 8.15 (t, J=7.9 Hz, 1H), 7.98-7.85 (m, 2H), 7.69 (d, J=5.0 Hz, 1H), 7.59-7.48 (m, 1H), 6.69-6.61 (m, 1H), 5.82 (s, 1H), 3.71 (s, 3H), 3.40-3.31 (m, 2H), 2.73 (s, 3H), 2.59-2.53 (m, 1H), 2.11-2.03 (m, 1H). The second eluting peak was designated as enantiomer 2 (white solid, 25 mg, 20%) (Example 88). LC-MS (ESI⁺): m/z=431.3. ¹H NMR (600 MHz, DMSO-d₆) δ 9.81 (s, 1H), 8.58 (d, J=5.0 Hz, 1H), 8.55 (d, J=1.5 Hz, 1H), 8.26-8.19 (m, 1H), 8.15 (t, J=7.9 Hz, 1H), 8.01-7.86 (m, 2H), 7.69 (d, J=5.0 Hz, 1H), 7.54 (d, J=2.2 Hz, 1H), 6.66-6.61 (m, 1H), 5.83 (s, 1H), 3.71 (s, 3H), 3.44-3.31 (m, 2H), 2.73 (s, 3H), 2.60-2.52 (m, 1H), 2.12-2.04 (m, 1H).

### Example 89. (R)-3-Hydroxy-1-methyl-3-(1-(6-(2-((1-methyl-1H-pyrazol-3-yl)amino)pyrimidin-4-yl)pyridin-2-yl)-1H-1,2,3-triazol-4-yl)pyrrolidin-2-one

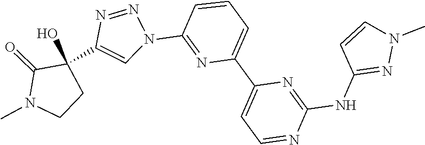

Step A. (R)-3-(1-(6-Chloro-4-methylpyridin-2-yl)-1H-1,2,3-triazol-4-yl)-3-hydroxy-1-methylpyrrolidin-2-one. (R)-3-(1-(6-Chloro-4-methylpyridin-2-yl)-1H-1,2,3-triazol-4-yl)-3-hydroxy-1-methylpyrrolidin-2-one was prepared in a manner analogous to (R)-3-(1-(6-bromopyridin-2-yl)-1H-1,2,3-triazol-4-yl)-3-hydroxy-1-methylpyrrolidin-2-one (Intermediate 51) using 2,6-dichloro-4-methylpyridine in place of 2,6-dibromopyridine.

Step B. (R)-3-Hydroxy-1-methyl-3-(1-(6-(2-((1-methyl-1H-pyrazol-3-yl)amino)pyrimidin-4-yl)pyridin-2-yl)-1H-1,2,3-triazol-4-yl)pyrrolidin-2-one. (R)-3-Hydroxy-1-methyl-3-(1-(6-(2-((1-methyl-1H-pyrazol-3-yl)amino)pyrimidin-4-yl)pyridin-2-yl)-1H-1,2,3-triazol-4-yl)pyrrolidin-2-one was prepared in a manner analogous to Example 87, Step A, using (R)-3-(1-(6-chloro-4-methylpyridin-2-yl)-1H-1,2,3-triazol-4-yl)-3-hydroxy-1-methylpyrrolidin-2-one in place of 3-(1-(6-chloropyridin-2-yl)-1H-imidazol-4-yl)-3-hydroxy-1-methylpyrrolidin-2-one to yield the title compound as yellow solid (10 mg, 9%). LCMS (ESI⁺): m/z=447.2. ¹H NMR (500 MHz, Methanol-d₄) δ 8.95 (s, 1H), 8.72 (d, J=5.9 Hz, 1H), 8.46 (s, 1H), 8.25 (s, 1H), 8.15-8.04 (m, 1H), 7.66 (d, J=2.4 Hz, 1H), 6.40 (s, 1H), 3.94 (s, 3H), 3.68-3.57 (m, 2H), 2.98-2.89 (m, 4H), 2.65 (d, J=0.8 Hz, 3H), 2.43 (ddd, J=13.3, 8.3, 6.5 Hz, 1H).

### Example 90. (3R)-3-Hydroxy-1-methyl-3-(3-(6-(2-((1-(pyrrolidin-3-yl)-1H-pyrazol-4-yl)amino)pyrimidin-4-yl)pyridin-2-yl)isoxazol-5-yl)pyrrolidin-2-one

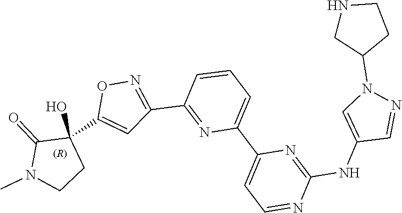

(3R)-3-Hydroxy-1-methyl-3-(3-(6-(2-((1-(pyrrolidin-3-yl)-1H-pyrazol-4-yl)amino)pyrimidin-4-yl)pyridin-2-yl)isoxazol-5-yl)pyrrolidin-2-one was prepared in a manner analogous to (R)-3-(3-(6-(2-((1H-pyrazol-3-yl)amino)pyrimidin-4-yl)pyridin-2-yl)isoxazol-5-yl)-3-hydroxy-1-methylpyrrolidin-2-one (Example 31) using tert-butyl 3-(4-amino-1H-pyrazol-1-yl)pyrrolidine-1-carboxylate in place of tert-butyl 3-amino-1H-pyrazole-1-carboxylate. The title compound was purified using SFC (Stationary phase: Chiralpak IA 5 μm 250×21 mm, Mobile phase: 50% methanol with 0.2% triethylamine, 50% CO₂) to yield a yellow solid (12%). MS (ESI⁺): m/z=488.0.

### Example 91: (R)-3-(5-(6-(2-((1-Ethyl-1H-pyrazol-4-yl)amino)pyrimidin-4-yl)pyridin-2-yl)isoxazol-3-yl)-3-hydroxy-1-methylpyrrolidin-2-one

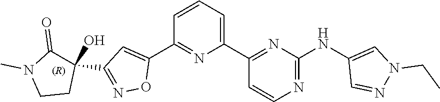

A mixture of (R)-3-hydroxy-1-methyl-3-[5-[6-(2-methylsulfonylpyrimidin-4-yl)-2-pyridyl]isoxazol-3-yl]pyrrolidin-2-one (Intermediate 21, 50 mg, 0.12 mmol), 1-ethyl-1H-pyrazol-4-amine (33 mg, 0.3 mmol), LiHMDS (0.6 mL, 1 M in THF, 0.6 mmol), and DMSO (1 mL, 14 mmol) was stirred under nitrogen for 2 hours at room temperature. The reaction mixture was diluted with water (50 mL) and washed with ethyl acetate (3×50 mL). The organic extracts were dried over MgSO₄, filtered, and concentrated under reduced pressure before diluting in 3 mL DMSO (+1 mL water) for purification by RP HPLC (NH₄OH Modifier) to yield (R)-3-(5-(6-(2-((1-ethyl-1H-pyrazol-4-yl)amino)pyrimidin-4-yl)pyridin-2-yl)isoxazol-3-yl)-3-hydroxy-1-methylpyrrolidin-2-one (5 mg, 9%). ¹H NMR (500 MHz, DMSO-d₆) δ 9.64 (s, 1H), 8.64 (d, J=5.0 Hz, 1H), 8.47 (d, J=8.1 Hz, 1H), 8.25 (t, J=7.8 Hz, 1H), 8.12 (dd, J=7.8, 1.0 Hz, 1H), 8.00 (s, 1H), 7.74 (d, J=5.0 Hz, 1H), 7.62 (s, 1H), 7.34 (s, 1H), 6.56 (s, 1H), 4.15 (q, J=7.4 Hz, 3H), 3.50-3.45 (m, 2H), 3.45-3.40 (m, 1H), 2.82 (s, 3H), 2.76-2.69 (m, 2H), 2.31-2.23 (m, 2H), 1.39 (t, J=7.3 Hz, 3H). MS (ESI⁺): m/z=447.6.

### Example 92: (3R,5R)-3-Hydroxy-1,5-dimethyl-3-(3-(6-(2-((1-methyl-1H-pyrazol-3-yl)amino)pyrimidin-4-yl)pyridin-2-yl)isoxazol-5-yl)pyrrolidin-2-one

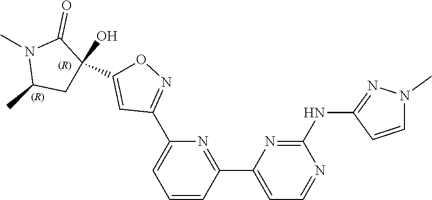

A mixture of (3R,5R)-3-(3-(6-bromopyridin-2-yl)isoxazol-5-yl)-3-hydroxy-1,5-dimethylpyrrolidin-2-one (Intermediate 35, 100 mg, 0.3 mmol), N-(1-methyl-1H-pyrazol-3-yl)-4-(trimethylstannyl)pyrimidin-2-amine (Intermediate 10, 153 mg, 0.453 mmol), Pd(PPh₃)₄ (16 mg, 0.01 mmol), DMF (2 mL, 26 mmol), and lithium chloride (14 mg, 0.3 mmol) was stirred under nitrogen in a microwave reactor at 130° C. for 1 hour. The reaction mixture was diluted in EtOAc and water and stirred vigorously with potassium fluoride (33 mg, 0.6 mmol) for 1 hour. The solids were filtered off before transferring the mixture to a separatory funnel for aqueous extraction with (3×75 mL) EtOAc and water (100 mL). The organic layer was dried with MgSO₄, filtered and concentrated under reduced pressure. The product was passed through a metal scavenging cartridge (thiol) with EtOAc and concentrated in 3.5 mL DMSO for purification by RP HPLC (NH₄OH Modifier) to yield (3R,5R)-3-hydroxy-1,5-dimethyl-3-(3-(6-(2-((1-methyl-1H-pyrazol-3-yl)amino)pyrimidin-4-yl)pyridin-2-yl)isoxazol-5-yl)pyrrolidin-2-one (19 mg, 14%). H NMR (500 MHz, DMSO-d₆) δ 9.89 (s, 1H), 8.65 (d, J=5.0 Hz, 1H), 8.47 (dd, J=7.7, 1.2 Hz, 1H), 8.21 (t, J=7.8 Hz, 1H), 8.15 (dd, J=7.8, 1.2 Hz, 1H), 7.82 (d, J=5.0 Hz, 1H), 7.62 (d, J=2.2 Hz, 1H), 7.16 (s, 1H), 6.77 (s, 1H), 6.72 (d, J=2.2 Hz, 1H), 3.78 (s, 3H), 3.71 (q, J=6.4 Hz, 1H), 2.85 (dd, J=13.4, 6.9 Hz, 1H), 2.82 (s, 3H), 1.92 (dd, J=13.4, 6.4 Hz, 1H), 1.31 (d, J=6.3 Hz, 3H). MS (ESI⁺): m/z=447.3.

### Example 93: (1R,4R,5S)-4-Hydroxy-2-methyl-4-(3-(6-(2-((1-methyl-1H-pyrazol-3-yl)amino)pyrimidin-4-yl)pyridin-2-yl)isoxazol-5-yl)-2-azabicyclo[3.1.0]hexan-3-one

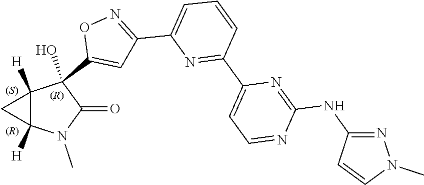

A mixture of (1R,4R,5S)-4-(3-(6-bromopyridin-2-yl)isoxazol-5-yl)-4-hydroxy-2-methyl-2-azabicyclo[3.1.0]hexan-3-one (Intermediate 33, 100 mg, 0.3 mmol), N-(1-methyl-1H-pyrazol-3-yl)-4-(trimethylstannyl)pyrimidin-2-amine (Intermediate 10) (154 mg, 0.456 mmol), Pd(PPh₃)₄ (16.5 mg, 0.014 mmol), DMF (2 mL, 26 mmol), and lithium chloride (14.5 mg, 0.3 mmol) was subjected to microwave irradiation under a nitrogen atmosphere at 130° C. for 1 hour. The reaction mixture was then diluted with EtOAc and water and stirred vigorously with potassium fluoride (33 mg, 0.6 mmol) for 1 hour. The solids were filtered off before transferring the mixture to a separatory funnel for aqueous extraction with (3×75 mL) EtOAc and water (100 mL). The organic extracts were dried with MgSO₄, filtered and concentrated under reduced pressure. The product was diluted with water and the precipitate was filtered off. After dissolving the precipitate with 3.5 mL DMSO, the solution purified by RP HPLC (NH₄OH Modifier) to yield (1R,4R,5S)-4-hydroxy-2-methyl-4-(3-(6-(2-((1-methyl-1H-pyrazol-3-yl)amino)pyrimidin-4-yl)pyridin-2-yl)isoxazol-5-yl)-2-azabicyclo[3.1.0]hexan-3-one (8.9 mg, 7%). ¹H NMR (500 MHz, DMSO-d₆) δ 9.88 (s, 1H), 8.64 (d, J=5.0 Hz, 1H), 8.47 (dd, J=7.6, 1.3 Hz, 1H), 8.21 (t, J=7.7 Hz, 1H), 8.18-8.14 (m, 1H), 7.82 (d, J=5.0 Hz, 1H), 7.61 (d, J=2.2 Hz, 1H), 7.18 (s, 1H), 6.72 (d, J=2.2 Hz, 1H), 6.67 (s, 1H), 3.77 (s, 3H), 3.47-3.43 (m, 1H), 2.86 (s, 3H), 2.12-2.06 (m, 1H), 1.00-0.94 (m, 1H), 0.85-0.80 (m, 1H). MS (ESI⁺): m/z=445.0.

### Example 94: (R)-4,4-Difluoro-3-hydroxy-1-methyl-3-(3-(6-(2-((1-methyl-1H-pyrazol-3-yl)amino)pyrimidin-4-yl)pyrazin-2-yl)isoxazol-5-yl)pyrrolidin-2-one

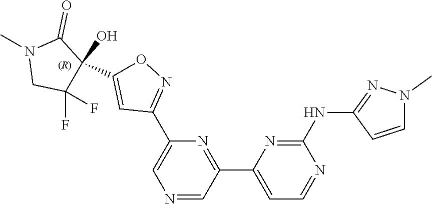

A mixture of (R)-3-(3-(6-chloropyrazin-2-yl)isoxazol-5-yl)-4,4-difluoro-3-hydroxy-1-methylpyrrolidin-2-one (Intermediate 37, 60 mg, 0.2 mmol), N-(1-methyl-1H-pyrazol-3-yl)-4-(trimethylstannyl)pyrimidin-2-amine (Intermediate 10, 61 mg, 0.2 mmol), [1,1′-bis(diphenylphosphino)ferrocene]dichloropalladium(II) (7 mg, 0.01 mmol), cuprous iodide (3.5 mg, 0.02 mmol) and DMF (2 mL, 26 mmol) were heated under a nitrogen atmosphere at 130° C. for 1 hour. The reaction mixture was diluted in water and ethyl acetate and stirred vigorously with potassium fluoride (263 mg, 4.5 mmol) for 1 hour. The solids were filtered off before extracting with EtOAc (50 mL) and water (3×50 mL). The organic extracts were dried over anhydrous MgSO₄, filtered, and concentrated under reduced pressure before being diluted in 3 mL DMSO for purification via RP HPLC (C18 50×100 mm 0-95% (20 mM NH₄OH in water)/acetonitrile) to yield (R)-4,4-difluoro-3-hydroxy-1-methyl-3-(3-(6-(2-((1-methyl-1H-pyrazol-3-yl)amino)pyrimidin-4-yl)pyrazin-2-yl)isoxazol-5-yl)pyrrolidin-2-one (2.4 mg, 3%). ¹H NMR (400 MHz, DMSO-d₆) δ 10.01 (s, 1H), 9.60 (s, 1H), 9.40 (s, 1H), 8.69 (d, J=4.9 Hz, 1H), 8.02 (s, 1H), 7.82 (d, J=5.0 Hz, 1H), 7.64-7.63 (m, 1H), 7.40 (s, 1H), 6.73-6.71 (m, 1H), 4.07-4.00 (m, 2H), 3.79 (s, 3H), 2.98 (s, 3H). MS (ESI⁺): m/z=470.1.

### Example 95: (R)-3-Hydroxy-1-methyl-3-(3-(6-(2-((1-methyl-1H-pyrazol-3-yl)amino)pyrimidin-4-yl)pyrazin-2-yl)isoxazol-5-yl)pyrrolidin-2-one

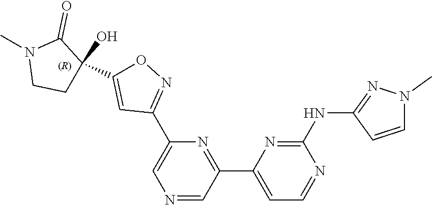

A mixture of (R)-3-(3-(6-chloropyrazin-2-yl)isoxazol-5-yl)-3-hydroxy-1-methylpyrrolidin-2-one (Intermediate 38, 10 mg, 0.03 mmol), N-(1-methyl-1H-pyrazol-3-yl)-4-(trimethylstannyl)pyrimidin-2-amine (Intermediate 10, 11.5 mg, 0.03 mmol), [1,1′-bis(diphenylphosphino)ferrocene]dichloropalladium(II) (1.25 mg, 0.0017 mmol), cuprous iodide (0.6 mg, 0.003 mmol), and DMF (1 mL, 13 mmol) were heated at 130° C. for 1 hour. The reaction mixture was diluted in water and ethyl acetate and stirred vigorously with potassium fluoride (296 mg, 5 mmol) for 1 hour. The solids were filtered off. The filtrate was extracted with EtOAc (50 mL) and water (3×50 mL). The organic extracts were dried over MgSO₄, filtered, and concentrated under reduced pressure before being diluted in 3 mL DMSO for purification via RP HPLC (C18 50×100 mm, 0-95% (20 mM NH₄OH in water)/acetonitrile). The product fractions were combined and passed through a carbonate silica cartridge before lyophilization to yield (R)-3-hydroxy-1-methyl-3-(3-(6-(2-((1-methyl-1H-pyrazol-3-yl)amino)pyrimidin-4-yl)pyrazin-2-yl)isoxazol-5-yl)pyrrolidin-2-one (13.1 mg, 15%) as a yellow solid. ¹H NMR (500 MHz, DMSO-d₆) δ 10.02 (s, 1H), 9.59 (s, 1H), 9.37 (s, 1H), 8.70 (d, J=5.0 Hz, 1H), 7.79 (d, J=4.9 Hz, 1H), 7.64 (d, J=2.2 Hz, 1H), 7.25 (s, 1H), 6.82 (s, 1H), 6.73-6.70 (m, 1H), 3.79 (s, 3H), 3.54-3.49 (m, 1H), 3.49-3.43 (m, 1H), 2.86 (s, 3H), 2.66-2.60 (m, 1H), 2.35-2.29 (m, 1H). MS (ESI⁺): m/z=434.1.

### Example 96: (R)-3-Hydroxy-1-methyl-3-(3-(6-(2-(((RS)-2,2,2-trifluoro-1-(1-methyl-1H-pyrazol-4-yl)ethyl)amino)pyrimidin-4-yl)pyridin-2-yl)isoxazol-5-yl)pyrrolidin-2-one

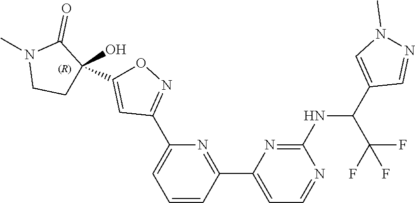

A mixture of (R)-3-(3-(6-(2-chloropyrimidin-4-yl)pyridin-2-yl)isoxazol-5-yl)-3-hydroxy-1-methylpyrrolidin-2-one (Intermediate 31, 50 mg, 0.13 mmol), 2,2,2-trifluoro-1-(1-methyl-1H-pyrazol-4-yl)ethan-1-amine (24.1 mg, 0.134 mmol), Cs₂CO₃ (87.6 mg, 0.27 mmol), rac-binap-Pd-G3 (13 mg, 0.013 mmol), and 1,4-dioxane (1.5 mL, 17.6 mmol) was heated under a nitrogen atmosphere at 100° C. for 18 hours. The reaction mixture was allowed to cool to room temperature and stirred for an additional 8 days. The reaction mixture was then diluted with water and extracted with EtOAc. The organic extracts were dried over MgSO₄, filtered and concentrated under reduced pressure before diluting with DMSO for purification by RP HPLC (TFA modifier) to yield (R)-3-hydroxy-1-methyl-3-(3-(6-(2-(((RS)-2,2,2-trifluoro-1-(1-methyl-1H-pyrazol-4-yl)ethyl)amino)pyrimidin-4-yl)pyridin-2-yl)isoxazol-5-yl)pyrrolidin-2-one (13.8 mg, 19%). ¹H NMR (500 MHz, DMSO-d₆) δ 8.61 (d, J=5.0 Hz, 1H), 8.40 (d, J=9.8 Hz, 1H), 8.21-8.17 (m, 1H), 8.17-8.14 (m, 1H), 7.95 (s, 1H), 7.79 (d, J=5.0 Hz, 1H), 7.69 (s, 1H), 7.16 (s, 1H), 6.25 (s, 1H), 3.84 (s, 3H), 3.53-3.48 (m, 1H), 3.48-3.42 (m, 1H), 2.86 (s, 3H), 2.66-2.59 (m, 1H), 2.33-2.27 (m, 1H). MS (ESI⁺): m/z=515.1.

### Example 97: (R)-3-(1-(6-(2-((3-Cyclopropoxy-1-(2,2-difluoroethyl)-1H-pyrazol-4-yl)amino)pyrimidin-4-yl)pyridin-2-yl)-1H-pyrazol-4-yl)-3-hydroxy-1-methylpyrrolidin-2-one

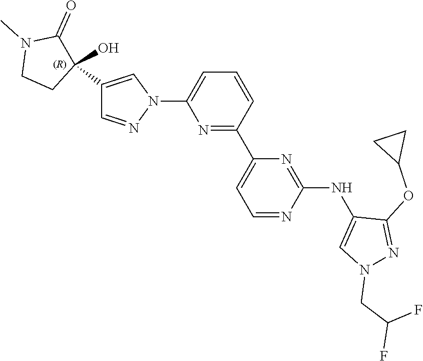

A mixture of (R)-3-hydroxy-1-methyl-3-(1-(6-(2-(methylsulfonyl)pyrimidin-4-yl)pyridin-2-yl)-1H-pyrazol-4-yl)pyrrolidin-2-one (Intermediate 22, 50 mg, 0.12 mmol), 3-(cyclopropoxy)-1-(2,2-difluoroethyl)pyrazol-4-amine (Intermediate 14, 123 mg, 0.6 mmol), TsOH (25 mg, 0.15 mmol), and 1,4-dioxane (1 mL) was heated under a nitrogen atmosphere at 100° C. for 6 hours. The product was dissolved in 3 mL of DMSO for purification by RP HPLC (NH₄₀H Modifier) to yield (R)-3-(1-(6-(2-((3-cyclopropoxy-1-(2,2-difluoroethyl)-1H-pyrazol-4-yl)amino)pyrimidin-4-yl)pyridin-2-yl)-1H-pyrazol-4-yl)-3-hydroxy-1-methylpyrrolidin-2-one (12.4 mg, 18%). ¹H NMR (500 MHz, DMSO-d₆) δ8.80 (d, J=0.8 Hz, 1H), 8.66-8.62 (m, 1H), 8.61 (d, J=5.0 Hz, 1H), 8.27-8.23 (m, 1H), 8.16 (t, J=7.9 Hz, 1H), 8.04 (dd, J=8.1, 1.0 Hz, 1H), 7.95 (s, 1H), 7.88 (d, J=0.8 Hz, 1H), 7.78 (d, J=5.0 Hz, 1H), 6.34 (tt, J=55.2, 3.9 Hz, 1H), 6.00 (s, 1H), 4.56-4.46 (m, 2H), 4.14-4.09 (m, 1H), 3.47-3.41 (m, 1H), 3.39-3.34 (m, 1H), 2.80 (s, 3H), 2.61-2.54 (m, 1H), 2.30-2.22 (m, 1H), 0.73-0.68 (m, 2H), 0.68-0.62 (m, 2H). MS (ESI⁺): m/z=538.5.

### Example 98: (R)-3-Hydroxy-1-methyl-3-(1-(6-(2-(pyrazolo[1,5-a]pyridin-3-ylamino)pyrimidin-4-yl)pyridin-2-yl)-1H-pyrazol-4-yl)pyrrolidin-2-one

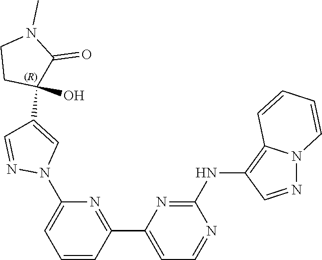

A mixture of (R)-3-hydroxy-1-methyl-3-(1-(6-(2-(methylsulfonyl)pyrimidin-4-yl)pyridin-2-yl)-1H-pyrazol-4-yl)pyrrolidin-2-one (Intermediate 22, 50 mg, 0.12 mmol), pyrazolo[1,5-a]pyridin-3-amine (80 mg, 0.6 mmol), TsOH (25 mg, 0.15 mmol), and 1,4-dioxane (1 mL) was added and stirred under nitrogen at 100° C. for 4 hours. The product was diluted with 50 mL of water and washed with ethyl acetate (3×50 mL). The organic extracts were dried over MgSO₄, filtered and concentrated under reduced pressure before dissolving in 3 mL of DMSO for purification by RP HPLC (TFA modifier) to yield (R)-3-hydroxy-1-methyl-3-(1-(6-(2-(pyrazolo[1,5-a]pyridin-3-ylamino)pyrimidin-4-yl)pyridin-2-yl)-1H-pyrazol-4-yl)pyrrolidin-2-one (23.1 mg, 39%) as a brown solid. ¹H NMR (500 MHz, DMSO-d₆) δ 9.68 (s, 1H), 8.82-8.81 (m, 1H), 8.68-8.65 (m, 1H), 8.59 (dt, J=7.0, 1.1 Hz, 1H), 8.51-8.43 (m, 1H), 8.29-8.22 (m, 1H), 8.19 (t, J=7.8 Hz, 1H), 8.06-8.03 (m, 1H), 7.89-7.88 (m, 1H), 7.86-7.83 (m, 1H), 7.83-7.81 (m, 1H), 7.14 (ddd, J=9.0, 6.6, 1.0 Hz, 1H), 6.85 (td, J=6.8, 1.4 Hz, 1H), 3.47-3.42 (m, 2H), 3.39-3.34 (m, 1H), 2.81 (s, 3H), 2.62-2.56 (m, 1H), 2.30-2.23 (m, 1H). MS (ESI⁺): m/z=468.1.

### Example 99: (R)-3-Hydroxy-1-methyl-3-(3-(4-methyl-6-(2-((1-methyl-1H-pyrazol-4-yl)amino)pyrimidin-4-yl)pyridin-2-yl)isoxazol-5-yl)pyrrolidin-2-one

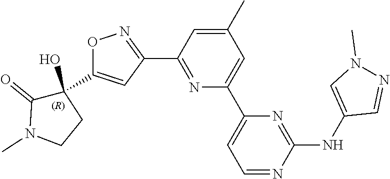

Step A: (E)-6-Bromo-4-methylpicolinaldehyde oxime. 6-Bromo-4-methylpicolinaldehyde (5.0 g, 2.5 mmol), hydroxylamine hydrochloride (191 mg, 2.75 mmol), sodium acetate (410 mg, 5 mmol) and EtOH (20 mL) were combined. The resulting mixture was stirred for 17 h. The residue was dissolved in ethyl acetate (30 mL), washed with water (20 mL×2), dried over anhydrous sodium sulfate to provide (E)-6-bromo-4-methylpicolinaldehyde oxime as white solid (443 mg, 82%). LCMS (ESI): Mass calcd. for C₇H₇BrN₂O 213.97 m/z, found 213.0 [M+H]⁺.

Step B: (Z)-6-Bromo-N-hydroxy-4-methylpicolinimidoyl chloride. (E)-6-Bromo-4-methylpicolinaldehyde oxime (443 mg, 2.1 mmol), NCS (357 mg, 2.7 mmol) and DMF (10 mL) were combined. The resulting mixture was stirred for 19 h at rt. The reaction mixture was then diluted with water (30 mL) and extracted with ethyl acetate (25 mL×3). The combined extracts were washed with brine (20 mL), dried over anhydrous Na₂SO₄, filtered, and concentrated to dryness in vacuo to give a yellow solid. The yellow solid was then subjected to silica gel chromatography (0-100% ethyl acetate/hexanes) to give (Z)-6-bromo-N-hydroxy-4-methylpicolinimidoyl chloride as an off-white solid (257.5 mg, 50%). LC-MS (ESI): Mass calcd. for C₇H₆BrClN₂O 249.93 m/z found 250 [M+H]⁺. ¹H NMR (400 MHz, MeOH-d₄) δ 7.75 (s, 1H), 7.52 (s, 1H), 2.41 (s, 3H).

Step C: rac-(R)-3-(3-(6-Bromo-4-methylpyridin-2-yl)isoxazol-5-yl)-3-hydroxy-1-methylpyrrolidin-2-one. (Z)-6-Bromo-N-hydroxy-4-methylpicolinimidoyl chloride (257.5 mg, 1.03 mmol), (3R)-3-ethynyl-3-hydroxy-1-methyl-pyrrolidin-2-one, (287.2 mg, 2.06 mmol), sodium bicarbonate (260.1 mg, 3.1 mmol), ethyl acetate (5 mL), and water (0.5 mL) were combined. The resulting mixture stirred for 19 h at room temperature. The reaction mixture was concentrated to dryness in vacuo to give a yellow residue. The yellow residue was then subjected to silica gel chromatography (0-10% MeOH/DCM) to give rac-(R)-3-(3-(6-bromo-4-methylpyridin-2-yl)isoxazol-5-yl)-3-hydroxy-1-methylpyrrolidin-2-one as a sticky, white solid (300 mg) and was used without further purification. LC-MS (ESI): Mass calcd. for C₁₄H₁₄BrN₃O₃ 351.02 m/z found 352.0 [M+H]⁺.

Step D: rac-(R)-3-Hydroxy-1-methyl-3-(3-(4-methyl-6-(2-(methylthio)pyrimidin-4-yl)pyridin-2-yl)isoxazol-5-yl)pyrrolidin-2-one. rac-(R)-3-(3-(6-Bromo-4-methylpyridin-2-yl)isoxazol-5-yl)-3-hydroxy-1-methylpyrrolidin-2-one (200 mg, 0.607 mmol), 2-(methylthio)-4-(tributylstannyl)pyrimidine (81.7 mg, 0.197 mmol) and Pd(PPh₃)₄ (41.3 mg, 0.035 mmol), were dissolved in DMF (1 mL, sparged). The mixture was subjected to microwave irradiation for 1.5 hours at 140° C. The reaction was then quenched by the addition of 10 g of KF, 10 mL of water, and 30 mL EtOAc. This biphasic mixture was stirred for 10 minutes at rt, after which the mixture was filtered through diatomaceous earth, and the filtrate transferred to a separatory funnel. The organic layer was extracted, dried over anhydrous sodium sulfate, filtered, and concentrated under reduced pressure. The residue was subjected to silica gel chromatography (0-10% MeOH/DCM) to afford rac-(R)-3-hydroxy-1-methyl-3-(3-(4-methyl-6-(2-(methylthio)pyrimidin-4-yl)pyridin-2-yl)isoxazol-5-yl)pyrrolidin-2-one as a foamy orange solid (59.7 mg, 84%). LC-MS (ESI): Mass calcd. For C₁₉H₁₉N₅O₃S 397.12 m/z found 398.10 [M+H]⁺.

Step E. rac-(R)-3-Hydroxy-1-methyl-3-(3-(4-methyl-6-(2-(methylsulfonyl)pyrimidin-4-yl)pyridin-2-yl)isoxazol-5-yl)pyrrolidin-2-one. rac-(R)-3-Hydroxy-1-methyl-3-(3-(4-methyl-6-(2-(methylthio)pyrimidin-4-yl)pyridin-2-yl)isoxazol-5-yl)pyrrolidin-2-one (59.7 mg, 0.15 mmol) was dissolved in acetone (0.315 mL), water (0.315 mL), THF (0.315 mL), and MeOH (0.315 mL). To this mixture was added potassium peroxymonosulfate (0.277 g, 0.451 mmol) and the resulting mixture was stirred for 17 hours at room temperature. To this mixture, ice water (5 mL) was added. After 10 minutes, the solids were removed by filtration, rinsed with additional water (100 mL), and dried for several hours under vacuum to yield the title compound as a white solid (26.5 mg, 41%). ¹H NMR (500 MHz, DMSO-d₆) δ 9.27-9.22 (m, 1H), 8.82-8.76 (m, 1H), 8.50-8.45 (m, 1H), 8.14-8.08 (m, 1H), 7.24 (s, 1H), 6.77 (s, 1H), 3.56 (s, 3H), 3.54-3.40 (m, 2H), 2.85 (s, 3H), 2.66-2.59 (m, 1H), 2.59-2.55 (m, 3H), 2.35-2.26 (m, 1H). LC-MS (ESI): Mass calcd. For C₁₉H₁₉N₅O₅S 429.1 m/z found 430.1 [M+H]⁺.

Step F. (R)-3-Hydroxy-1-methyl-3-(3-(4-methyl-6-(2-((1-methyl-1H-pyrazol-4-yl)amino)pyrimidin-4-yl)pyridin-2-yl)isoxazol-5-yl)pyrrolidin-2-one. rac-(R)-3-Hydroxy-1-methyl-3-(3-(4-methyl-6-(2-(methylsulfonyl)pyrimidin-4-yl)pyridin-2-yl)isoxazol-5-yl)pyrrolidin-2-one (27.5 mg, 0.06 mmol) and 1-methyl-1H-pyrazol-4-amine (18.7 mg, 0.19 mmol) were dissolved in DMSO (anhydrous, 0.64 mL), and TFA (0.015 mL, 0.19 mmol). The resulting reaction mixture was subjected to microwave irradiation at 155° C. for 90 min. After that time, the reaction mixture was cooled to room temperature, filtered through a syringe filter to provide (R)-3-hydroxy-1-methyl-3-(3-(4-methyl-6-(2-((1-methyl-1H-pyrazol-4-yl)amino)pyrimidin-4-yl)pyridin-2-yl)isoxazol-5-yl)pyrrolidin-2-one. This compound was purified by RP-HPLC via acidic conditions using column C7 to afford the title compound as its TFA salt as a yellow solid (3.9 mg, 11%). 1H NMR (500 MHz, DMSO-d₆) δ 9.62 (s, 1H), 8.64-8.59 (m, 1H), 8.32-8.29 (m, 1H), 8.02-8.00 (m, 1H), 7.98 (s, 1H), 7.76-7.71 (m, 1H), 7.58 (s, 1H), 7.14 (s, 1H), 6.75 (s, 1H), 3.85 (s, 3H), 2.85 (s, 3H), 2.62-2.58 (m, 1H), 2.55 (s, 4H), 2.34-2.25 (m, 1H). LC-MS (ESI): Mass calcd. for C₂₂H₂₂N₈O₃ 446.2 m/z, found 447.2 [M+H]⁺. The TFA salt could be converted to the corresponding free base by passage through a carbonate cartridge.

### Example 100: (R)-3-Hydroxy-1-methyl-3-(3-(4-methyl-6-(2-((1-methyl-1H-pyrazol-3-yl)amino)pyrimidin-4-yl)pyridin-2-yl)isoxazol-5-yl)pyrrolidin-2-one

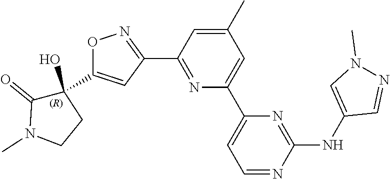

(R)-3-Hydroxy-1-methyl-3-(3-(4-methyl-6-(2-((1-methyl-1H-pyrazol-3-yl)amino)pyrimidin-4-yl)pyridin-2-yl)isoxazol-5-yl)pyrrolidin-2-one and its TFA salt were prepared in a manner analogous to (R)-3-hydroxy-1-methyl-3-(3-(4-methyl-6-(2-((1-methyl-1H-pyrazol-4-yl)amino)pyrimidin-4-yl)pyridin-2-yl)isoxazol-5-yl)pyrrolidin-2-one (Example 99) using 1-methyl-1H-pyrazol-3-amine in place of 1-methyl-1H-pyrazol-4-amine to yield the title compound as its TFA salt as a yellow solid (2 mg, 15%). ¹H NMR (500 MHz, DMSO-d₆) δ 9.86 (s, 1H), 8.65-8.59 (m, 1H), 8.31-8.26 (m, 1H), 8.03-7.98 (m, 1H), 7.81-7.77 (m, 1H), 7.63-7.58 (m, 1H), 7.15 (s, 1H), 6.72-6.67 (m, 1H), 3.77 (s, 3H), 2.85 (s, 3H), 2.65-2.59 (m, 2H), 2.54 (s, 4H), 2.38-2.26 (m, 2H). LC-MS (ESI): Mass calcd. for C₂₂H₂₂N₈O₃ 446.2 m/z, found 447.1 [M+H]⁺.

### Example 101: (R)-3-(3-(6-(6-Chloro-2-((1-methyl-1H-pyrazol-3-yl)amino)pyrimidin-4-yl)pyridin-2-yl)isoxazol-5-yl)-3-hydroxy-1-methylpyrrolidin-2-one

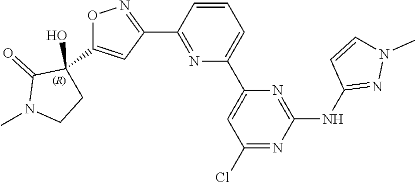

Step A: 4,6-Dichloro-N-(1-methyl-1H-pyrazol-3-yl)pyrimidin-2-amine. A mixture of 1-ethyl-1H-pyrazol-3-amine (4.5 mL, 52 mmol) and THF (80 mL) was cooled to −72° C. LiHMDS (40 mL, 40 mmoL, 1 M in THF) was added and the resulting mixture stirred for 30 min at −72° C. 4-Chloro-2-(methylsulfonyl)pyrimidine (5.9 g, 33 mmol) and THF (20 mL) were added and the resulting mixture was stirred for 2 h at −72° C. Ice water was added and then the mixture was stirred for 1 h resulting in formation of a precipitate. The precipitate was collected by filtration and washed with ethyl acetate and dried under vacuum to afford 4,6-dichloro-N-(1-methyl-1H-pyrazol-3-yl)pyrimidin-2-amine (3.65 g, 57%). MS (ESI⁺): m/z=244.0.

Step B: 4-Chloro-N-(1-methyl-1H-pyrazol-3-yl)-6-(tributylstannyl)pyrimidin-2-amine. A mixture of 4,6-dichloro-N-(1-methyl-1H-pyrazol-3-yl)pyrimidin-2-amine (7.65 g, 21.9 mmol), LiCl (2.9 g, 68 mmol), 1,4-dioxane (180 mL), 1,1,1,2,2,2-hexabutyldistannane (18.76 g, 32.34 mmol), Pd₂(dba)₃ (509 mg, 0.556 mmol) and tricyclohexylphosphine (321 mg, 1.15 mmol) was heated at 100° C. for 16 h. The reaction mixture was then treated with sat. aq. KF (150 mL) and stirred for 16 h at room temperature. The mixture was then extracted with EtOAc (230 mL×3) and the organic layer was washed with brine (180 mL), dried over anhydrous Na₂SO₄, filtered, and concentrated to dryness in vacuo to give a brown oil, The oil was then was subjected to FCC (0-100% EtOAc/pet ether) to give 4-chloro-N-(1-methyl-1H-pyrazol-3-yl)-6-(tributylstannyl)pyrimidin-2-amine (1.9 g, 9%). The mixture was then extracted with EtOAc (230 mL×3) and the organic layer was washed with brine (180 mL), dried over anhydrous Na₂SO₄, filtered, and concentrated to dryness in vacuo to give a brown oil. The oil was then was subjected to FCC (0-100% EtOAc/pet ether) to give 4-chloro-N-(1-methyl-1H-pyrazol-3-yl)-6-(tributylstannyl)pyrimidin-2-amine (1.9 g, 9%). MS (ESI⁺): m/z=498.1.

Step C: (R)-3-(3-(6-(6-Chloro-2-((1-methyl-1H-pyrazol-3-yl)amino)pyrimidin-4-yl)pyridin-2-yl)isoxazol-5-yl)-3-hydroxy-1-methylpyrrolidin-2-one. (R)-3-(3-(6-Bromopyridin-2-yl)isoxazol-5-yl)-3-hydroxy-1-methylpyrrolidin-2-one (Step A of Intermediate 13, 165 mg, 0.488 mmol), 4-chloro-N-(1-methyl-1H-pyrazol-3-yl)-6-(tributylstannyl)pyrimidin-2-amine (300 mg, 0.602 mmol), 1,4-dioxane (6 mL) and Pd(dppf)Cl₂ (36 mg, 0.049 mmol) was heated for 16 hours at 100° C. After cooling to room temperature, the reaction was then diluted with water (10 mL), extracted with ethyl acetate (15 mL×3). The combined extracts washed with brine (8 mL), dried over anhydrous Na₂SO₄, filtered, and concentrated to dryness in vacuo to give a yellow solid. The yellow solid was subjected to preparative HPLC (Phenomenex C18 (75 mm×30 mm×3 μm) column, (eluent: 30-60% (v/v) CH₃CN and water with 0.05% NH₃ in H₂O+10 mM NH₄HCO₃)) to afford (R)-3-(3-(6-(6-chloro-2-((1-methyl-1H-pyrazol-3-yl)amino)pyrimidin-4-yl)pyridin-2-yl)isoxazol-5-yl)-3-hydroxy-1-methylpyrrolidin-2-one (65.6 mg, 28%) as a yellow solid. MS (ESI) calcd. for C₂₁H₁₉ClN₈O₃, 466.13 m/z, found 467.1 [M+H]⁺. ¹H NMR (400 MHz, DMSO-d₆) δ 10.40 (s, 1H), 8.46-8.42 (m, 1H), 8.25-8.17 (m, 2H), 7.83 (s, 1H), 7.67-7.63 (m, 1H), 7.26 (s, 1H), 6.79 (s, 1H), 6.70 (br s, 1H), 3.79 (s, 3H), 3.54-3.42 (m, 2H), 2.86 (s, 3H), 2.69-2.60 (m, 1H), 2.35-2.25 (m, 1H).

### Example 102 and Example 103: (S)-3-(5-(6-(2-((5-Chloro-1-methyl-1H-pyrazol-4-yl)amino)pyrimidin-4-yl)pyridin-2-yl)-1,3,4-thiadiazol-2-yl)-3-hydroxy-1-methylpyrrolidin-2-one and (R)-3-(5-(6-(2-((5-chloro-1-methyl-1H-pyrazol-4-yl)amino)pyrimidin-4-yl)pyridin-2-yl)-1,3,4-thiadiazol-2-yl)-3-hydroxy-1-methylpyrrolidin-2-one

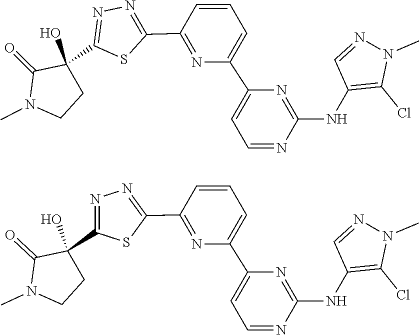

Step A: 6-Bromo-N′-(1-methyl-2-oxopyrrolidine-3-carbonyl)picolinohydrazide. A mixture of 1-methyl-2-oxopyrrolidine-3-carboxylic acid (500 mg, 3.49 mmol), DMF (7.5 mL), 6-bromopicolinohydrazide (750 mg, 3.47 mmol), HATU (1.33 g, 3.50 mmol), and DIEA (1.85 mL, 11.2 mmol) was stirred at rt for 24 h resulting in the formation of a white precipitate. The precipitate was collected by filtration, washed with MeOH, and dried under vacuum to afford the first crop of 6-bromo-N′-(1-methyl-2-oxopyrrolidine-3-carbonyl)picolinohydrazide (214 mg, 15%). The filtrate was concentrated and the residue was then subjected to HPLC ((Xtimate C18 150×40 mm, 10 μm), 1-30% ACN/water (FA)) to give a 2^{nd} crop of 6-bromo-N′-(1-methyl-2-oxopyrrolidine-3-carbonyl)picolinohydrazide as a yellow solid (520 mg, 43%). LCMS (ESI⁺): mass calcd. for C₁₂H₁₃BrN₄O₃ 341.16 m/z, found 342.9 [M+H]⁺.

Step B: 3-(5-(6-Bromopyridin-2-yl)-1,3,4-thiadiazol-2-yl)-1-methylpyrrolidin-2-one. A mixture of 6-bromo-N′-(3-hydroxy-1-methyl-2-oxopyrrolidine-3-carbonyl)picolinohydrazide (500 mg, 1.47 mmol), 1,4-dioxane (15 mL), and 2,4-bis(4-methoxyphenyl)-1,3,2,4-dithiadiphosphetane 2,4-disulfide (950 mg, 2.35 mmol) was heated at 60° C. for 1 h. The mixture was cooled to rt, diluted with ethyl acetate, washed with H₂O, dried over anhydrous Na₂SO₄, filtered and concentrated to give a yellow oil. The oil was then subjected to FCC (0-100% EA/petroleum ether) to give 3-(5-(6-bromopyridin-2-yl)-1,3,4-thiadiazol-2-yl)-1-methylpyrrolidin-2-one as a yellow solid (300 mg, 37%). LCMS (ESI⁺): mass calcd. for C₁₂H₁₁BrN₄OS 339.21 m/z, found 339.0 [M+H]⁺.

Step C: 3-(5-(6-Bromopyridin-2-yl)-1,3,4-thiadiazol-2-yl)-3-hydroxy-1-methylpyrrolidin-2-one. A mixture of 3-(5-(6-bromopyridin-2-yl)-1,3,4-thiadiazol-2-yl)-1-methylpyrrolidin-2-one (300 mg, 0.88 mmol), triazabicyclodecane (125 mg, 0.898 mmol) and DMF (7 mL) was stirred at 25° C. for 1 hour under an 02 atmosphere (15 psi). The reaction mixture was then diluted with ethyl acetate, washed with H₂O, dried over anhydrous Na₂SO₄, filtered, and concentrated to dryness in vacuo to give 3-(5-(6-bromopyridin-2-yl)-1,3,4-thiadiazol-2-yl)-3-hydroxy-1-methylpyrrolidin-2-one as a yellow solid (150 mg, 44%). LCMS (ESI⁺): mass calcd. for C₁₂H₁₁BrN₄O₂S 355.21 m/z, found 356.7 [M+H]⁺.

Step D: 3-(5-(6-(2-((5-Chloro-1-methyl-1H-pyrazol-4-yl)amino)pyrimidin-4-yl)pyridin-2-yl)-1,3,4-thiadiazol-2-yl)-3-hydroxy-1-methylpyrrolidin-2-one. A mixture of 3-(5-(6-bromopyridin-2-yl)-1,3,4-thiadiazol-2-yl)-3-hydroxy-1-methylpyrrolidin-2-one (180 mg, 0.507 mmol), 1,4-dioxane (10 mL), N-(5-chloro-1-methyl-1H-pyrazol-4-yl)-4-(tributylstannyl)pyrimidin-2-amine (270 mg, 0.541 mmol, Intermediate 43) and Pd(dppf)Cl₂ (90 mg, 0.12 mmol) were heated at 100° C. for 5 h. The mixture was then diluted with ethyl acetate, washed with H₂O, dried over anhydrous Na₂SO₄, filtered and concentrated to give a red oil. The oil was subjected to HPLC ((Welch Xtimate C18 150×30 mm, 5 μm), 33-63% MeCN/water (0.05% NH₃ in H₂O+NH₄HCO₃)) to give 3-(5-(6-(2-((5-chloro-1-methyl-1H-pyrazol-4-yl)amino)pyrimidin-4-yl)pyridin-2-yl)-1,3,4-thiadiazol-2-yl)-3-hydroxy-1-methylpyrrolidin-2-one as a yellow solid (130 mg, 53%). LCMS (ESI⁺): mass calcd. for C₂₀H₁₈ClN₉O₂S 483.93 m/z, found 484.1 [M+1]⁺.

Step E: (S)-3-(5-(6-(2-((5-Chloro-1-methyl-1H-pyrazol-4-yl)amino)pyrimidin-4-yl)pyridin-2-yl)-1,3,4-thiadiazol-2-yl)-3-hydroxy-1-methylpyrrolidin-2-one and (R)-3-(5-(6-(2-((5-chloro-1-methyl-1H-pyrazol-4-yl)amino)pyrimidin-4-yl)pyridin-2-yl)-1,3,4-thiadiazol-2-yl)-3-hydroxy-1-methylpyrrolidin-2-one. The (R) and (S) enantiomers of 3-(5-(6-(2-((5-chloro-1-methyl-1H-pyrazol-4-yl)amino)pyrimidin-4-yl)pyridin-2-yl)-1,3,4-thiadiazol-2-yl)-3-hydroxy-1-methylpyrrolidin-2-one were separated by SFC (DAICEL CHIRALCEL OJ column, 10 μm, 250×30 mm; 30% (v/v) MeOH (containing 0.1% of NH₃ in H₂O)/CO₂)). The first eluting isomer was designated (R)-3-(5-(6-(2-((5-chloro-1-methyl-1H-pyrazol-4-yl)amino)pyrimidin-4-yl)pyridin-2-yl)-1,3,4-thiadiazol-2-yl)-3-hydroxy-1-methylpyrrolidin-2-one (23.5 mg, 22.9%). ¹H NMR (400 MHz, DMSO-d₆) δ 9.11 (s, 1H), 8.68-8.57 (m, 1H), 8.46-8.32 (m, 2H), 8.30-8.18 (m, 1H), 7.80 (s, 1H), 7.74-7.67 (m, 1H), 7.23 (s, 1H), 3.84 (s, 3H), 3.57-3.48 (m, 2H), 2.93-2.86 (m, 1H), 2.84 (s, 3H), 2.41-2.32 (m, 1H). The second eluting isomer was designated (S)-3-(5-(6-(2-((5-chloro-1-methyl-1H-pyrazol-4-yl)amino)pyrimidin-4-yl)pyridin-2-yl)-1,3,4-thiadiazol-2-yl)-3-hydroxy-1-methylpyrrolidin-2-one (33.19 mg, 31.68%). ¹H NMR (400 MHz, DMSO-d₆) δ 9.01 (s, 1H), 8.68-8.54 (m, 1H), 8.45-8.32 (m, 2H), 8.30-8.19 (m, 1H), 7.79 (s, 1H), 7.74-7.67 (m, 1H), 7.22 (s, 1H), 3.86 (s, 3H), 3.56-3.50 (m, 2H), 2.93-2.86 (m, 1H), 2.85 (s, 3H), 2.42-2.32 (m, 1H).

### Example 104 and Example 105: (R)-3-(3-(6-(2-((5-Chloro-1-methyl-1H-pyrazol-4-yl)amino)pyrimidin-4-yl)pyridin-2-yl)-1H-pyrazol-5-yl)-3-hydroxy-1-methylpyrrolidin-2-one and (S)-3-(3-(6-(2-((5-chloro-1-methyl-1H-pyrazol-4-yl)amino)pyrimidin-4-yl)pyridin-2-yl)-1H-pyrazol-5-yl)-3-hydroxy-1-methylpyrrolidin-2-one

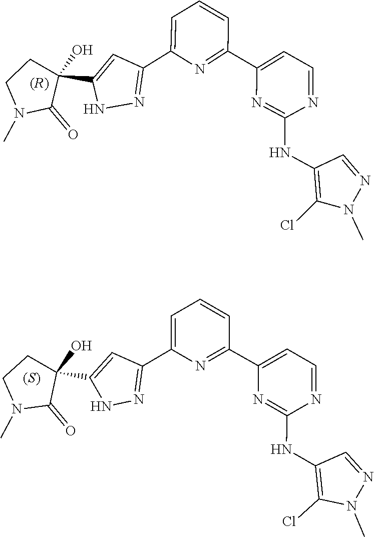

A mixture of 3-(3-(6-(2-((5-chloro-1-methyl-1H-pyrazol-4-yl)amino)pyrimidin-4-yl)pyridin-2-yl)-1-((2-(trimethylsilyl)ethoxy)methyl)-1H-pyrazol-5-yl)-3-hydroxy-1-methylpyrrolidin-2-one (Intermediate 45, 110 mg, 0.19 mmol) and TFA (1.0 mL, 13 mmol) was stirred for 16 h at room temperature. The mixture was concentrated to dryness under reduced pressure to afford a red oil, which was subjected to preparative RP HPLC (Welch Xtimate C18 column, 5 μm, 150×30 mm; 23-53% (v/v) MeCN and water (0.05% NH₃ in H₂O+10 mM NH₄HCO₃)) to give, after lyophillization, (R,S)-3-(3-(6-(2-((5-chloro-1-methyl-1H-pyrazol-4-yl)amino)pyrimidin-4-yl)pyridin-2-yl)-1H-pyrazol-5-yl)-3-hydroxy-1-methylpyrrolidin-2-one as a yellow solid. The mixture of enantiomers was separated by SFC (DAICEL CHIRALCEL OD-H column, 5 μm, 250×30 mm; 40% (v/v) CO₂/MeOH (0.1% NH₃ in H₂O)) to give two products. The first eluting peak was designated enantiomer 1 (Example 104) of (R,S)-3-(3-(6-(2-((5-chloro-1-methyl-1H-pyrazol-4-yl)amino)pyrimidin-4-yl)pyridin-2-yl)-1H-pyrazol-5-yl)-3-hydroxy-1-methylpyrrolidin-2-one, a yellow solid after lyophilization (11.3 mg, 22%). ¹H NMR (400 MHz, DMSO-d₆) δ 13.66-12.82 (m, 1H), 9.00 (s, 1H), 8.71-8.47 (m, 1H), 8.26-7.7 (m, 5H), 7.05-6.84 (m, 1H), 6.35-5.91 (m, 1H), 3.83 (s, 3H), 3.44-3.39 (m, 2H), 2.85-2.64 (m, 4H), 2.31-2.08 (m, 1H). MS (ESI⁺): m/z=466.2. The second eluting peak was designated enantiomer 2 (Example 105) of (R,S)-3-(3-(6-(2-((5-chloro-1-methyl-1H-pyrazol-4-yl)amino)pyrimidin-4-yl)pyridin-2-yl)-1H-pyrazol-5-yl)-3-hydroxy-1-methylpyrrolidin-2-one, a yellow solid after lyophilization (9.4 mg, 18%). ¹H NMR (400 MHz, DMSO-d₆) δ 13.61-12.79 (m, 1H), 9.00 (s, 1H), 8.58 (s, 1H), 8.28-7.71 (m, 5H), 7.07-6.86 (m, 1H), 6.37-5.91 (m, 1H), 3.83 (s, 3H), 3.46-3.41 (m, 2H), 2.90-2.65 (m, 4H), 2.28-1.99 (m, 1H). MS (ESI⁺): m/z=466.2.

### Example 106: (*S)-3-(1-(6-(2-((5-Chloro-1-methyl-1H-pyrazol-4-yl)amino)pyrimidin-4-yl)pyridin-2-yl)-1H-imidazol-2-yl)-3-hydroxy-1-methylpyrrolidin-2-one

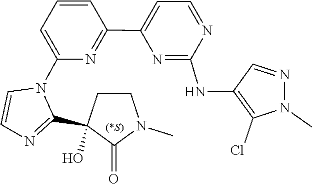

A mixture of 3-(1-(6-bromopyridin-2-yl)-1H-imidazol-2-yl)-3-hydroxy-1-methylpyrrolidin-2-one (90 mg, 0.267 mmol), N-(5-chloro-1-methyl-1H-pyrazol-4-yl)-4-(tributylstannyl)pyrimidin-2-amine (140 mg, 0.28 mmol, Intermediate 43), Pd(PPh₃)₄ (32 mg, 0.028 mmol), TEA (75 μL, 0.54 mmol), and xylenes (3 mL) was sparged with N₂ for 5 min and heated at 110° C. for 16 h. The reaction mixture was then cooled to room temperature and 5 mL of 2 M KF solution was added. This mixture which was then allowed to stir for 2 h at room temperature and then was concentrated to give a residue. The residue was subjected to preparative RP HPLC (Boston Green ODS (150×30 mm×5 μm) column, (eluent: 10-47% (v/v) CH₃CN/H₂O with 0.05% FA)) and lyophilized to afford 3-(1-(6-(2-((5-chloro-1-methyl-1H-pyrazol-4-yl)amino)pyrimidin-4-yl)pyridin-2-yl)-1H-imidazol-2-yl)-3-hydroxy-1-methylpyrrolidin-2-one as a yellow solid. The mixture of enantiomers were separated to SFC (DAICEL CHIRALCEL OD-H (250×30 mm, 5 μm), 45% EtOH (0.1% NH₃ in H₂O)/55% CO₂) to give the (R) and (S) enantiomers of 3-(1-(6-(2-((5-chloro-1-methyl-1H-pyrazol-4-yl)amino)pyrimidin-4-yl)pyridin-2-yl)-1H-imidazol-2-yl)-3-hydroxy-1-methylpyrrolidin-2-one. The second eluting peak, designated enantiomer 2 (Example 106) of 3-(1-(6-(2-((5-chloro-1-methyl-1H-pyrazol-4-yl)amino)pyrimidin-4-yl)pyridin-2-yl)-1H-imidazol-2-yl)-3-hydroxy-1-methylpyrrolidin-2-one was concentrated under reduced pressure, and subjected to preparative RP HPLC (Boston Green ODS (150×30 mm, 5 μm) column, (eluent: 10-40% (v/v) CH₃CN/H₂O with 0.05% FA)) to afford a yellow solid, (*S)-3-(1-(6-(2-((5-chloro-1-methyl-1H-pyrazol-4-yl)amino)pyrimidin-4-yl)pyridin-2-yl)-1H-imidazol-2-yl)-3-hydroxy-1-methylpyrrolidin-2-one (10 mg, 8%). ¹H NMR (400 MHz, DMSO-d₆) δ8.53-8.46 (m, 1H), 8.41 (d, J=8.0 Hz, 1H), 8.14 (t, J=8.0 Hz, 1H), 7.85 (s, 1H), 7.75 (d, J=8.0 Hz, 1H), 7.59-7.49 (m, 2H), 7.09 (s, 1H), 3.87 (s, 3H), 3.50-3.33 (m, 2H), 3.14-3.00 (m, 1H), 2.76 (s, 3H), 2.36-2.24 (m, 1H). MS (ESI⁺): m/z=466.2 [M+H]⁺.

### Example 107 and Example 108: (R)-3-(1-(6-(2-((5-Chloro-1-methyl-1H-pyrazol-4-yl)amino)pyrimidin-4-yl)pyridin-2-yl)-1H-imidazol-4-yl)-3-hydroxy-1-methylpyrrolidin-2-one and (S)-3-(1-(6-(2-((5-chloro-1-methyl-1H-pyrazol-4-yl)amino)pyrimidin-4-yl)pyridin-2-yl)-1H-imidazol-4-yl)-3-hydroxy-1-methylpyrrolidin-2-one

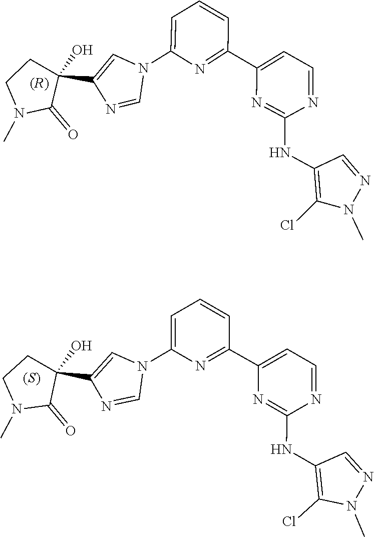

A mixture of 3-(1-(6-bromopyridin-2-yl)-1H-imidazol-4-yl)-3-hydroxy-1-methylpyrrolidin-2-one (Intermediate 44, 220 mg, 0.65 mmol), N-(5-chloro-1-methyl-1H-pyrazol-4-yl)-4-(tributylstannyl)pyrimidin-2-amine (Intermediate 43, 340 mg, 0.68 mmol), and TEA (200 μL, 1.4 mmol) was dissolved in 5 mL toluene. The resulting mixture was sparged with N₂ for 5 min. Pd(PPh₃)₄ (70 mg, 0.06 mmol) was then added. The resulting mixture was heated at 110° C. for 12 h. The mixture was cooled to room temperature and 5 mL 2 M aqueous KF solution was added. This mixture was then allowed to stir vigorously for 2 h at room temperature. The mixture was then filtered through a pad of diatomaceous earth and washed with 50 mL water and 50 mL DCM. The aqueous phase was then extracted with a 3:1 mixture of chloroform and isopropanol (30 mL×3). The organic layers were then collected, dried over anhydrous sodium sulfate, filtered and concentrated to give a residue. The residue was purified by preparative RP HPLC (Boston Prime C18 (150×30 mm×5 μm) column (eluent: 28-58% (v/v) CH₃CN/H₂O with 0.05% NH₃ in H₂O+10 mM NH₄HCO₃)) to afford 3-(1-(6-(2-((5-chloro-1-methyl-1H-pyrazol-4-yl)amino)pyrimidin-4-yl)pyridin-2-yl)-1H-imidazol-4-yl)-3-hydroxy-1-methylpyrrolidin-2-one as a yellow solid. The mixture of enantiomers were separated by SFC, DAICEL CHIRALCEL OD-H (250×30 mm, 5 μm)); Mobile phase: A: Supercritical CO₂, B: IPA (0.1% NH₃ in H₂O), A:B=55:45 at 80 mL/min) to give two products. The first eluting peak was designated enantiomer 1 (Example 107) of 3-(1-(6-(2-((5-chloro-1-methyl-1H-pyrazol-4-yl)amino)pyrimidin-4-yl)pyridin-2-yl)-1H-imidazol-4-yl)-3-hydroxy-1-methylpyrrolidin-2-one (60 mg, 39%). ¹H NMR (400 MHz, DMSO-d₆) δ 9.07 (s, 1H), 8.76-8.51 (m, 2H), 8.33-8.11 (m, 2H), 8.08-7.90 (m, 2H), 7.89-7.63 (m, 2H), 5.93 (s, 1H), 3.83 (s, 3H), 3.48-3.41 (m, 2H), 2.80 (s, 3H), 2.70-2.55 (m, 1H), 2.24-2.08 (m, 1H). MS (ESI⁺): m/z=466.1. The second eluting peak was designated enantiomer 2 (Example 108) of 3-(1-(6-(2-((5-chloro-1-methyl-1H-pyrazol-4-yl)amino)pyrimidin-4-yl)pyridin-2-yl)-1H-imidazol-4-yl)-3-hydroxy-1-methylpyrrolidin-2-one (55 mg, 35%). 1H NMR (400 MHz, DMSO-d₆) δ 9.07 (s, 1H), 8.68-8.54 (m, 2H), 8.29-8.10 (m, 2H), 8.06-7.92 (m, 2H), 7.85-7.66 (m, 2H), 5.93 (s, 1H), 3.83 (s, 3H), 3.51-3.39 (m, 2H), 2.80 (s, 3H), 2.69-2.57 (m, 1H), 2.25-2.08 (m, 1H). MS (ESI⁺): m/z=466.2.

### Example 109: (R)-3-(1-(6-(2-((5-Chloro-1-methyl-1H-pyrazol-4-yl)amino)pyrimidin-4-yl)pyridin-2-yl)-1H-1,2,3-triazol-4-yl)-4,4-difluoro-3-hydroxy-1-methylpyrrolidin-2-one

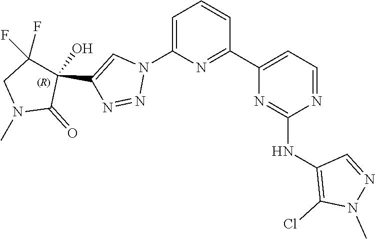

A mixture of (R)-3-(1-(6-bromopyridin-2-yl)-1H-1,2,3-triazol-4-yl)-4,4-difluoro-3-hydroxy-1-methylpyrrolidin-2-one (Intermediate 48, 120 mg, 0.321 mmol), N-(5-chloro-1-methyl-1H-pyrazol-4-yl)-4-(tributylstannyl)pyrimidin-2-amine (Intermediate 43, 165 mg, 0.331 mmol), Pd(PPh₃)₄ (40 mg, 0.035 mmol) and TEA (100 μL, 0.72 mmol) in 5 mL toluene was sparged with N₂ for 5 min and heated at 100° C. for 16 h. The mixture was concentrated under reduced pressure and subjected to preparative HPLC (Welch Xtimate C18 (150×30 mm×5 μm) column (eluent: 30-60% (v/v) CH₃CN/H₂O with 0.05% NH₃ in H₂O+10 mM NH₄HCO₃)) to afford the title compound (60 mg, 37%) as a yellow solid. MS (ESI): Mass calcd. for C₂₀H₁₇ClF₂N₁₀O₂, 502.9; m/z found, 503.2 [M+H]⁺. ¹H NMR (400 MHz, DMSO-d₆) δ 9.19-9.01 (m, 2H), 8.61 (d, J=4.8 Hz, 1H), 8.45-8.21 (m, 3H), 7.88 (d, J=4.8 Hz, 1H), 7.79 (s, 1H), 7.45 (s, 1H), 4.13-3.92 (m, 2H), 3.84 (s, 3H), 2.97 (s, 3H).

### Example 110: (R)-3-(3-(6-(2-((6,7-Dihydro-4H-pyrazolo[5,1-c][1,4]oxazin-3-yl)amino)pyrimidin-4-yl)pyridin-2-yl)isoxazol-5-yl)-4,4-difluoro-3-hydroxy-1-methylpyrrolidin-2-one

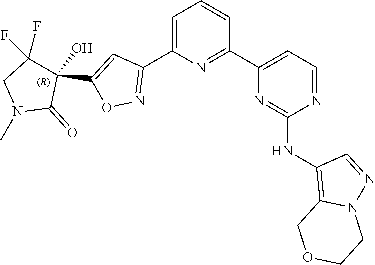

Step A. N-(4-Chloropyrimidin-2-yl)-6,7-dihydro-4H-pyrazolo[5,1-c][4]oxazin-3-amine. N-(4-Chloropyrimidin-2-yl)-6,7-dihydro-4H-pyrazolo[5,1-c][1,4]oxazin-3-amine was prepared analogous to 4-chloro-N-(1-methyl-1H-pyrazol-3-yl)pyrimidin-2-amine (Example 25 Step B) using 6,7-dihydro-4H-pyrazolo[5,1-c][1,4]oxazin-3-amine in place of 1-methyl-1H-pyrazol-3-amine.

Step B. N-(4-(Tributylstannyl)pyrimidin-2-yl)-6,7-dihydro-4H-pyrazolo[5,1-c][1,4]oxazin-3-amine. N-(4-Chloropyrimidin-2-yl)-6,7-dihydro-4H-pyrazolo[5,1-c][1,4]oxazin-3-amine (455 mg, 1.8 mmol), LiCl (465 mg, 11.0 mmol), and 1,4-dioxane (10 mL) were added to a 50 mL flask fitted with a reflux condenser and 1,1,1,2,2,2-hexabutyldistannane (3.2 g, 5.5 mmol) was added. The flask was evacuated and refilled with N₂ and treated with Pd₂(dba)₃ (83 mg, 0.091 mmol) and tricyclohexylphosphine (52 mg, 0.19 mmol). The flask was evacuated and refilled with N₂ and heated at 120° C. for 16 h. The reaction mixture was then treated with sat. aq. KF (30 mL) and stirred for 1 h at room temperature. The mixture was then extracted with EtOAc (30 mL×3) and the organic layer was washed with brine (30 mL), dried over anhydrous Na₂SO₄, filtered, and concentrated to dryness in vacuo to give a brown oil. The brown oil was subjected to silica-gel chromatography (0 to 50% ethyl acetate in petroleum ether) to afford N-(4-(tributylstannyl)pyrimidin-2-yl)-6,7-dihydro-4H-pyrazolo[5,1-c][1,4]oxazin-3-amine as a yellow oil (420 mg, 45%). LCMS (ESI): Mass calcd. for C₂₂H₃₇N₅OSn 507.2 m/z, found 506.0 [M+H]⁺. ¹H NMR (400 MHz, DMSO-d₆) δ 8.74 (s, 1H), 8.20-8.06 (m, 1H), 7.60 (s, 1H), 6.88-6.64 (m, 1H), 4.75 (s, 2H), 4.10-3.97 (m, 4H), 1.65-1.40 (m, 6H), 1.33-1.22 (m, 6H), 1.15-0.97 (m, 6H), 0.83 (t, J=7.2 Hz, 9H).

Step C. (R)-3-(3-(6-(2-((6,7-Dihydro-4H-pyrazolo[5,1-c][1,4]oxazin-3-yl)amino)pyrimidin-4-yl)pyridin-2-yl)isoxazol-5-yl)-4,4-difluoro-3-hydroxy-1-methylpyrrolidin-2-one. (R)-3-(3-(6-Bromopyridin-2-yl)isoxazol-5-yl)-4,4-difluoro-3-hydroxy-1-methylpyrrolidin-2-one (Intermediate 7, Step A, 100 mg, 0.267 mmol), N-(4-(tributylstannyl)pyrimidin-2-yl)-6,7-dihydro-4H-pyrazolo[5,1-c][1,4]oxazin-3-amine (029-2, 140 mg, 0.277 mmol), Pd(PPh₃)₄ (31 mg, 0.027 mmol), TEA (84 mg, 0.83 mmol) and toluene (3 mL) were added to a 10 mL round-bottomed flask. The resultant mixture was heated overnight at 110° C. under N₂ before cooling to room-temperature. The reaction mixture was then treated with sat. aq. KF (5 mL) and stirred for 1 h at room temperature. The mixture was then extracted with EtOAc (15 mL×3) and the organic layer was washed with brine (3 mL), dried over anhydrous Na₂SO₄, filtered, and concentrated to dryness in vacuo to give a black oil. The black oil was subjected to preparative HPLC (Waters xbridge (150×25 mm×10 μm) column (eluent: 18-48% (v/v) CH₃CN and H₂O with 0.05% NH₃ in H₂O+10 mM NH₄HCO₃)) to afford the title compound (45 mg, 32%) as a yellow solid. MS (ESI): Mass calcd. for C₂₃H₂₀F₂N₈O₄, 510.5; m/z found, 511.3 [M+H]⁺. ¹H NMR (400 MHz, DMSO-d₆) δ 9.15 (br. s., 1H), 8.62-8.55 (m, 1H), 8.44-8.35 (m, 1H), 8.23-8.14 (m, 2H), 7.97 (s, 1H), 7.84-7.63 (m, 2H), 7.30 (s, 1H), 4.80 (s, 2H), 4.14-3.98 (m, 6H), 2.97 (s, 3H).

### Example 111: (R)-3-Hydroxy-1-methyl-3-(5-(6-(2-((2-methylpyridin-3-yl)amino)pyrimidin-4-yl)pyridin-2-yl)isoxazol-3-yl)pyrrolidin-2-one

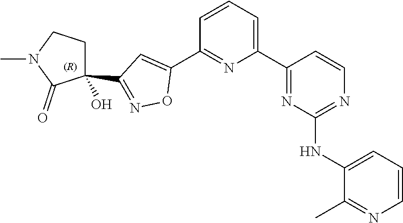

(R)-3-Hydroxy-1-methyl-3-(5-(6-(2-((2-methylpyridin-3-yl)amino)pyrimidin-4-yl)pyridin-2-yl)isoxazol-3-yl)pyrrolidin-2-one was prepared in a manner analogous to Example 109 using N-(2-methylpyridin-3-yl)-4-(tributylstannyl)pyrimidin-2-amine (Intermediate 46) in place of N-(5-chloro-1-methyl-1H-pyrazol-4-yl)-4-(tributylstannyl)pyrimidin-2-amine and (R)-3-(3-(6-chloropyridin-2-yl)isoxazol-5-yl)-4,4-difluoro-3-hydroxy-1-methylpyrrolidin-2-one in place of (R)-3-(1-(6-bromopyridin-2-yl)-1H-1,2,3-triazol-4-yl)-4,4-difluoro-3-hydroxy-1-methylpyrrolidin-2-one (46%). MS (ESI): Mass calcd. for C₂₃H₂₁N₇O₃, 443.5; m/z found, 444.2 [M+H]⁺. ¹H NMR (400 MHz, DMSO-d₆) δ 9.38 (s, 1H), 8.72-8.61 (m, 1H), 8.35-8.08 (m, 5H), 7.89-7.83 (m, 1H), 7.50-7.40 (m, 1H), 7.34 (s, 1H), 6.57 (br. s., 1H), 3.49-3.39 (m, 2H), 2.81 (s, 3H), 2.74-2.65 (m, 1H), 2.59-2.53 (m, 3H), 2.31-2.20 (m, 1H).

### Example 112: (R)-3-Hydroxy-3-(3-(6-(2-((2-methoxypyrimidin-5-yl)amino)pyrimidin-4-yl)pyridin-2-yl)isoxazol-5-yl)-1-methylpyrrolidin-2-one

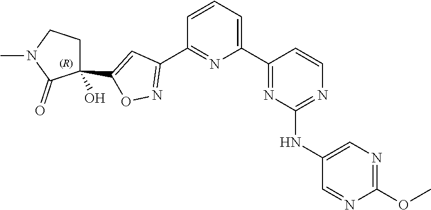

2-Methoxypyrimidin-5-amine (140 mg, 1.12 mmol) and THF (5 mL) were added to 25 mL three neck flask, which was subsequently evacuated and refilled with N₂, and charged with LiHMDS (1.6 mL, 1.6 mmoL, 1 M in THF). After 20 min, the resulting mixture was treated with (R)-3-(3-(6-(2-chloropyrimidin-4-yl)pyridin-2-yl)isoxazol-5-yl)-3-hydroxy-1-methylpyrrolidin-2-one (Intermediate 31, 200 mg, 0.538 mmol) and THF (5 mL). The resulting mixture was heated at 45° C. for 16 hrs. The mixture was concentrated to dryness in vacuo to afford a yellow solid. The yellow solid was then subjected to HPLC (Waters, Boston Prime 5 μm C18, 150×30 mm, 37-67% (v/v) ACN/(H₂O with 0.05% NH₃ in H₂O and 10 mM NH₄HCO₃)) to yield the title compound as a yellow solid (35 mg, 13%). MS (ESI): Mass calcd. for C₂₂H₂₀N₈O₄, 460.5; m/z found, 461.2 [M+H]⁺. ¹H NMR (400 MHz, DMSO-d₆) δ 9.89 (s, 1H) 8.99 (s, 2H) 8.68-8.74 (m, 1H) 8.39-8.45 (m, 1H) 8.15-8.27 (m, 2H) 7.88-7.94 (m, 1H) 7.19 (s, 1H) 6.79 (s, 1H) 3.92 (s, 3H) 3.41-3.55 (m, 2H) 2.86 (s, 3H) 2.58-2.66 (m, 1H) 2.26-2.34 (m, 1H).

### Example 113: (R)-3-Hydroxy-3-(3-(6-(2-((2-methoxypyrimidin-4-yl)amino)pyrimidin-4-yl)pyridin-2-yl)isoxazol-5-yl)-1-methylpyrrolidin-2-one

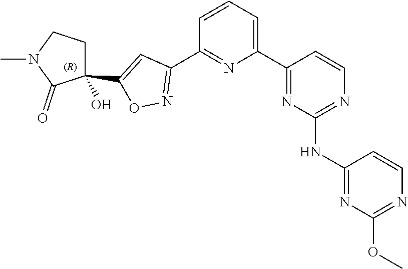

(R)-3-Hydroxy-3-(3-(6-(2-((2-methoxypyrimidin-4-yl)amino)pyrimidin-4-yl)pyridin-2-yl)isoxazol-5-yl)-1-methylpyrrolidin-2-one was made in a manner analogous to Example 112 using 2-methoxypyrimidin-4-amine in place of 2-methoxypyrimidin-5-amine to yield the title compound (20%). MS (ESI): Mass calcd. for C₂₂H₂₀N₈O₄, 460.5; m/z found, 461.2 [M+H]⁺. 1H NMR (400 MHz, DMSO-d₆) δ 10.61 (s, 1H) 8.84-8.88 (m, 1H) 8.52-8.57 (m, 1H) 8.45-8.49 (m, 1H) 8.18-8.27 (m, 2H) 8.07-8.14 (m, 2H) 7.21 (s, 1H) 6.80 (s, 1H) 3.92 (s, 3H) 3.41-3.55 (m, 2H) 2.86 (s, 3H) 2.59-2.66 (m, 1H) 2.27-2.35 (m, 1H).

### Example 114: (R)-3-Hydroxy-1-methyl-3-(3-(6-(2-(((*R)-6-methyl-6,7-dihydro-4H-pyrazolo[5,1-c][1,4]oxazin-2-yl)amino)pyrimidin-4-yl)pyridin-2-yl)isoxazol-5-yl)pyrrolidin-2-one

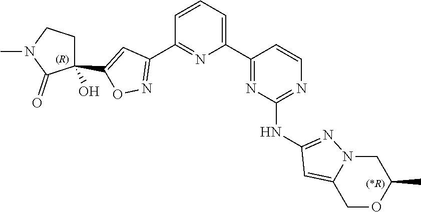

Step A. 1-(5-(Hydroxymethyl)-3-nitro-1H-pyrazol-1-yl)propan-2-ol. (5-Nitro-1H-pyrazol-3-yl)methanol (1.5 g, 10 mmol), Cs₂CO₃ (683 mg, 2.10 mmol) and 2-methyloxirane (8 mL) were combined and the mixture was stirred at rt for 16 h. The reaction mixture was then diluted with water (20 mL), extracted with ethyl acetate (30 mL×3), and the combined organic extracts washed with brine (40 mL), dried over anhydrous Na₂SO₄, filtered, and concentrated to dryness to give a white solid. The solid was subjected to preparative HPLC (Welch Xtimate C18(150×30 mm×5 μm) column (eluent: 10-40% (v/v) CH₃CN/H₂O with NH₃ in H₂O and NH₄HCO₃)) to afford 1-(5-(hydroxymethyl)-3-nitro-1H-pyrazol-1-yl)propan-2-ol (700 mg, 99%) as a white solid. MS (ESI⁺) calcd. For C₇H₁₁N₃O₄, 201.07 m/z, found 202.2 [M+H]⁺.

Step B. 6-Methyl-2-nitro-6,7-dihydro-4H-pyrazolo[5,1-c][1,4]oxazine. 1-(5-(Hydroxymethyl)-3-nitro-1H-pyrazol-1-yl)propan-2-ol (200 mg, 0.994 mmol), 4-methylbenzene-1-sulfonyl chloride (288 mg, 1.51 mmol), KOH (336 mg, 5.99 mmol), 1,4-dioxane (4 mL), and H₂O (2 mL) were combined and the resultant mixture was heated for 16 hours at 100° C. The mixture was cooled to room temperature and concentrated to dryness to give a white solid. The white solid was then subjected to silica gel chromatography (0-30% ethyl acetate/petroleum ether) to give 6-methyl-2-nitro-6,7-dihydro-4H-pyrazolo[5,1-c][1,4]oxazine (110 mg, 55%) as a white solid. MS (ESI⁺) calcd. for C₇H₉N₃O₃, 183.1 m/z, found 184.1 [M+H]⁺. ¹H NMR (400 MHz, DMSO-d₆) δ 6.89 (s, 1H), 5.01-4.91 (m, 1H), 4.81-4.71 (m, 1H), 4.36-4.26 (m, 1H), 4.16-4.04 (m, 1H), 3.95-3.84 (m, 1H), 3.35 (s, 1H), 1.33-1.29 (m, 3H).

Step C. 6-Methyl-6,7-dihydro-4H-pyrazolo[5,1-c][1,4]oxazin-2-amine. 6-Methyl-2-nitro-6,7-dihydro-4H-pyrazolo[5,1-c][1,4]oxazine (110 mg, 0.601 mmol) and MeOH (5 mL) were added to an oven-dried and nitrogen-purged flask, and the resulting mixture was treated with wet Pd/C (80 mg, 50% in water). The flask was subsequently evacuated and refilled with H₂. The resulting mixture stirred for 16 h at room temperature. The suspension was filtered through a pad of diatomaceous earth and the solvent was concentrated to dryness under reduced pressure to afford 6-methyl-6,7-dihydro-4H-pyrazolo[5,1-c][1,4]oxazin-2-amine (60 mg, 65%) as a yellow solid. MS (ESI⁺) calcd. for C₇H₁₁N₃O, 153.1 m/z, found 154.1 [M+H]⁺. ¹H NMR (400 MHz, DMSO-d₆) δ 6.89 (s, 1H), 5.01-4.91 (m, 1H), 4.81-4.71 (m, 1H), 4.36-4.26 (m, 1H), 4.16-4.04 (m, 1H), 3.95-3.84 (m, 1H), 1.33-1.29 (m, 3H).

Step D. (R)-3-Hydroxy-1-methyl-3-(3-(6-(2-((6-methyl-6,7-dihydro-4H-pyrazolo[5,1-c][1,4]oxazin-2-yl)amino)pyrimidin-4-yl)pyridin-2-yl)isoxazol-5-yl)pyrrolidin-2-one. (R)-3-(3-(6-(2-Chloropyrimidin-4-yl)pyridin-2-yl)isoxazol-5-yl)-3-hydroxy-1-methylpyrrolidin-2-one (Intermediate 31, 88 mg, 0.24 mmol), 6-methyl-6,7-dihydro-4H-pyrazolo[5,1-c][1,4]oxazin-2-amine (35 mg, 0.23 mmol), Cs₂CO₃ (235 mg, 0.721 mmol), and 1.4-dioxane (2 mL) were added to a 50 mL flask. The resulting mixture was purged with N₂ and treated with BrettPhos Pd G3 (21 mg, 0.023 mmol) and BrettPhos (38 mg, 0.071 mmol). The resultant mixture was purged with N₂ and heated at 100° C. for 16 hours, before cooling it to rt. The reaction was then diluted with water (20 mL), extracted with ethyl acetate (35 mL×3), and the combined extracts washed with brine (15 mL), dried over anhydrous Na₂SO₄, filtered, and concentrated to dryness in vacuo to give a yellow solid. The yellow solid was then subjected to HPLC (Waters, Boston Prime 5 μm C18, 150×30 mm, eluting with 40-70% (v/v) ACN/H₂O with 0.05% NH₃ in H₂O and 10 mM NH₄HCO₃)) to afford (R)-3-hydroxy-1-methyl-3-(3-(6-(2-((6-methyl-6,7-dihydro-4H-pyrazolo[5,1-c][1,4]oxazin-2-yl)amino)pyrimidin-4-yl)pyridin-2-yl)isoxazol-5-yl)pyrrolidin-2-one as a white solid (10 mg, 8%).

Step E. (R)-3-Hydroxy-1-methyl-3-(3-(6-(2-(((*R)-6-methyl-6,7-dihydro-4H-pyrazolo[5,1-c][1,4]oxazin-2-yl)amino)pyrimidin-4-yl)pyridin-2-yl)isoxazol-5-yl)pyrrolidin-2-one. The diastereomers of (R)-3-hydroxy-1-methyl-3-(3-(6-(2-((6-methyl-6,7-dihydro-4H-pyrazolo[5,1-c][1,4]oxazin-2-yl)amino)pyrimidin-4-yl)pyridin-2-yl)isoxazol-5-yl)pyrrolidin-2-one (15 mg, 0.031 mmol) were separated by chiral SFC (DAICEL CHIRALCEL OD-H (250×30 mm×5 μm), 60% IPA with 0.1% NH₃ in H₂O/40% CO₂). The title compound was eluted as the first peak and evaporated to dryness to afford the product (5 mg, 97% purity) as a yellow solid. The product was then further subjected to SFC (DAICEL CHIRALCEL OD (250×30 mm×10 μm), 55% IPA with 0.1% NH₃ in H₂O/45% CO₂) to afford the title compound (1.48 mg, 10%) as a yellow solid. MS (ESI): Mass calcd. for C₂₄H₂₄N₈O₄, 488.5; m/z found, 489.2 [M+H]⁺. ¹H NMR (400 MHz, DMSO-d₆) δ 9.46 (s, 1H), 8.64-8.59 (m, 1H), 8.47-8.42 (m, 1H), 8.19-8.09 (m, 2H), 7.80-7.75 (m, 1H), 7.11 (s, 1H), 6.56-6.46 (m, 2H), 4.97-4.87 (m, 1H), 4.79-4.70 (m, 1H), 4.11-3.99 (m, 2H), 3.68-3.61 (m, 1H), 3.52-3.39 (m, 2H), 2.85 (s, 3H), 2.68-2.56 (m, 1H), 2.35-2.26 (m, 1H), 1.33-1.30 (m, 3H).

### Example 115: (R)-3-Hydroxy-1-methyl-3-(3-(6-(2-(((*S)-6-methyl-6,7-dihydro-4H-pyrazolo[5,1-c][1,4]oxazin-2-yl)amino)pyrimidin-4-yl)pyridin-2-yl)isoxazol-5-yl)pyrrolidin-2-one

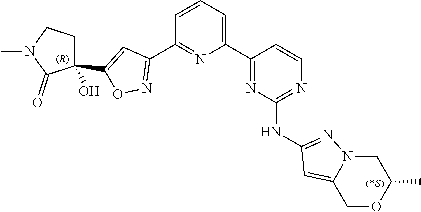

(R)-3-Hydroxy-1-methyl-3-(3-(6-(2-(((*S)-6-methyl-6,7-dihydro-4H-pyrazolo[5,1-c][1,4]oxazin-2-yl)amino)pyrimidin-4-yl)pyridin-2-yl)isoxazol-5-yl)pyrrolidin-2-one was prepared during the synthesis of (R)-3-hydroxy-1-methyl-3-(3-(6-(2-(((*R)-6-methyl-6,7-dihydro-4H-pyrazolo[5,1-c][1,4]oxazin-2-yl)amino)pyrimidin-4-yl)pyridin-2-yl)isoxazol-5-yl)pyrrolidin-2-one (Example 114) and eluted as the second peak during SFC purification to yield the title compound as a yellow solid (2.71 mg, 17%). MS (ESI): Mass calcd. for C₂₄H₂₄N₈O₄, 488.5; m/z found, 489.2 [M+H]⁺. ¹H NMR (400 MHz, DMSO-d₆) δ 9.48 (br s, 1H), 8.64-8.60 (m, 1H), 8.47-8.42 (m, 1H), 8.20-8.09 (m, 2H), 7.81-7.76 (m, 1H), 7.11 (s, 1H), 6.58-6.48 (m, 2H), 4.96-4.89 (m, 1H), 4.78-4.71 (m, 1H), 4.11-4.00 (m, 2H), 3.70-3.59 (m, 1H), 3.54-3.38 (m, 2H), 2.85 (s, 3H), 2.68-2.56 (m, 1H), 2.36-2.27 (m, 1H), 1.33-1.30 (m, 3H).

### Example 116: (R)-4,4-Difluoro-3-hydroxy-1-methyl-3-(3-(6-(2-(((*R)-4-methyl-6,7-dihydro-4H-pyrazolo[5,1-c][1,4]oxazin-3-yl)amino)pyrimidin-4-yl)pyridin-2-yl)isoxazol-5-yl)pyrrolidin-2-one

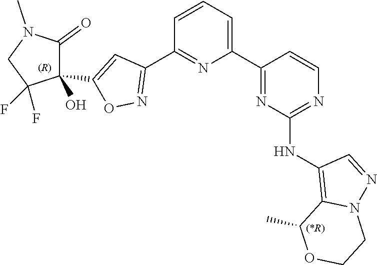

(R)-4,4-Difluoro-3-hydroxy-1-methyl-3-(3-(6-(2-(methylsulfonyl)pyrimidin-4-yl)pyridin-2-yl)isoxazol-5-yl)pyrrolidin-2-one (Intermediate 7, 100 mg, 0.222 mmol), (*R)-4-methyl-6,7-dihydro-4H-pyrazolo[5,1-c][1,4]oxazin-3-amine (Intermediate 70, 90 mg, 0.59 mmol), TsOH·H₂O (65 mg, 0.34 mmol) and 1,4-dioxane (3 mL) were combined. The resultant mixture was heated overnight at 115° C. before cooling to room-temperature. The reaction mixture was directly subjected to preparative HPLC (Waters xbridge (150×25 mm×10 μm) column (eluent: 15-45% (v/v) CH₃CN and H₂O with 0.05% NH₃ in H₂O+10 mM NH₄HCO₃)) to afford the title compound as a yellow solid. The solid was further purified by SFC (DAICEL CHIRALCEL OD, 250×30 mm, 10 μm, 35% (v/v) MeOH (0.1% NH₃ in H₂O)/65% CO₂) to afford the title compound (15.86 mg, 14%) as a yellow solid. MS (ESI): Mass calcd. for C₂₄H₂₂F₂N₈O₄, 524.5; m/z found, 525.3 [M+H]⁺. ¹H NMR (400 MHz, DMSO-d₆) δ 9.01 (br. s., 1H), 8.62-8.53 (m, 1H), 8.36 (br. s., 1H), 8.24-8.13 (m, 2H), 7.98 (s, 1H), 7.79-7.73 (m, 1H), 7.56 (br.s., 1H), 7.30 (s, 1H), 4.96 (q, J=6.4 Hz, 1H), 4.25-4.17 (m, 1H), 4.15-3.90 (m, 5H), 2.97 (s, 3H), 1.30 (d, J=6.4 Hz, 3H).

### Example 117: (R)-4,4-Difluoro-3-hydroxy-1-methyl-3-(1-(6-(2-((2-methylpyridin-4-yl)amino)pyrimidin-4-yl)pyridin-2-yl)-1H-1,2,3-triazol-4-yl)pyrrolidin-2-one

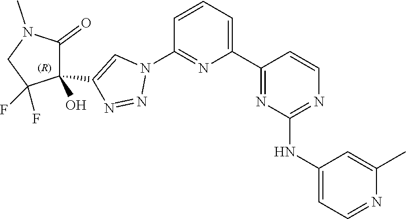

(R)-4,4-Difluoro-3-hydroxy-1-methyl-3-(1-(6-(2-((2-methylpyridin-4-yl)amino)pyrimidin-4-yl)pyridin-2-yl)-1H-1,2,3-triazol-4-yl)pyrrolidin-2-one was prepared in a manner analogous to Example 109 using N-(2-methylpyridin-4-yl)-4-(tributylstannyl)pyrimidin-2-amine (Intermediate 47) in place of N-(5-chloro-1-methyl-1H-pyrazol-4-yl)-4-(tributylstannyl)pyrimidin-2-amine (50 mg, 29%). MS (ESI): Mass calcd. for C₂₂H₁₉F₂N₉O₂, 479.4; m/z found, 480.2 [M+H]⁺. ¹H NMR (400 MHz, DMSO-d₆) δ 10.24 (s, 1H), 9.11 (s, 1H), 8.81 (d, J=5.2 Hz, 1H), 8.52 (d, J=7.6 Hz, 1H), 8.40 (t, J=8.0 Hz, 1H), 8.34-8.26 (m, 2H), 8.21 (s, 1H), 8.10 (d, J=5.2 Hz, 1H), 7.79-7.65 (m, 2H), 4.14-3.90 (m, 2H), 2.97 (s, 3H), 2.43 (s, 3H).

### Example 118 and Example 119: (R)-3-Hydroxy-1-methyl-3-(1-(6-(2-(((R)-1-(1-methyl-1H-pyrazol-4-yl)ethyl)amino)pyrimidin-4-yl)pyridin-2-yl)-1H-1,2,3-triazol-4-yl)pyrrolidin-2-one and (R)-3-hydroxy-1-methyl-3-(1-(6-(2-(((S)-1-(1-methyl-1H-pyrazol-4-yl)ethyl)amino)pyrimidin-4-yl)pyridin-2-yl)-1H-1,2,3-triazol-4-yl)pyrrolidin-2-one

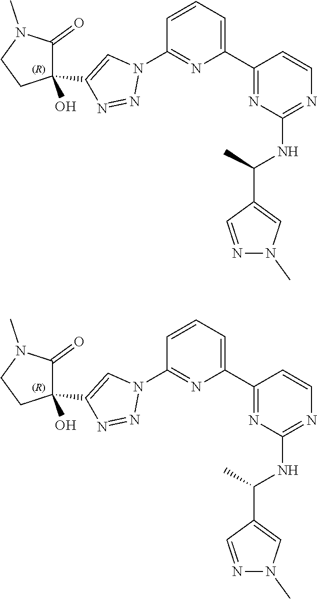

To a solution of (R)-3-(1-(6-(2-chloropyrimidin-4-yl)pyridin-2-yl)-1H-1,2,3-triazol-4-yl)-3-hydroxy-1-methylpyrrolidin-2-one (75 mg, 0.202 mmol, Intermediate 32) in DMSO (1.5 mL) were added 1-(1-methyl-1H-pyrazol-4-yl)ethanamine (30 mg, 0.24 mmol) and DIEA (80 mg, 0.619 mmol), sequentially. The resulting mixture was heated to 110° C. for 12 h, then cooled to room temperature and purified by reverse-phase HPLC (Xtimate C18 column, 5 μm, 30×150 mm; 23-53% ACN/H₂O (with 0.05% NH₃ and 10 mM NH₄HCO₃)) to afford, after lyophilization, a mixture of diastereomers as a white solid. The diastereomers were separated by chiral SFC (DAICEL CHIRALPAK AD, 10 μm, 30×250 mm, isocratic elution: 70% MeOH (0.1% of 25% aqueous NH₃), 30% CO₂) to provide Example 118 as the first-eluting diastereomer (21 mg, 26%) and Example 119 as the second-eluting diastereomer (20 mg, 24%). Example 118: MS (ESI): Mass calcd. for C₂₂H₂₄N₁₀O₂, 460.5; m/z found, 461.2 [M+H]⁺. ¹H NMR (400 MHz, DMSO-d₆) δ 8.92 (s, 1H), 8.58-8.37 (m, 2H), 8.34-8.27 (m, 1H), 8.27-8.21 (m, 1H), 7.65-7.55 (m, 3H), 7.41 (s, 1H), 6.35 (s, 1H), 5.38-5.14 (m, 1H), 3.77 (s, 3H), 3.49 (t, J=6.8 Hz, 2H), 2.83 (s, 3H), 2.82-2.74 (m, 1H), 2.34-2.21 (m, 1H), 1.49 (d, J=6.8 Hz, 3H). Example 119: MS (ESI): Mass calcd. for C₂₂H₂₄N₁₀O₂, 460.5; m/z found, 461.2 [M+H]⁺. ¹H NMR (400 MHz, DMSO-d₆) δ 8.92 (s, 1H), 8.59-8.38 (m, 2H), 8.34-8.27 (m, 1H), 8.27-8.21 (m, 1H), 7.67-7.56 (m, 3H), 7.40 (s, 1H), 6.35 (s, 1H), 5.41-5.09 (m, 1H), 3.77 (s, 3H), 3.49 (t, J=6.8 Hz, 2H), 2.83 (s, 3H), 2.81-2.74 (m, 1H), 2.34-2.21 (m, 1H), 1.49 (d, J=6.8 Hz, 3H).

### Example 120 (R)-4,4-Difluoro-3-hydroxy-1-methyl-3-(1-(6-(2-((1-methyl-1H-pyrazol-3-yl)amino)pyrimidin-4-yl)pyridin-2-yl)-1H-1,2,3-triazol-4-yl)pyrrolidin-2-one

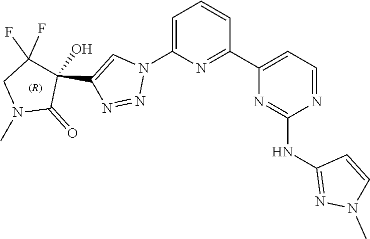

To a mixture of (R)-3-(1-(6-bromopyridin-2-yl)-1H-1,2,3-triazol-4-yl)-4,4-difluoro-3-hydroxy-1-methylpyrrolidin-2-one (60 mg, 0.160 mmol, Intermediate 48), N-(1-methyl-1H-pyrazol-3-yl)-4-(tributylstannyl)pyrimidin-2-amine (140 mg, 0.241 mmol, Intermediate 24) and TEA (33 mg, 0.326 mmol) in toluene (3 mL) was added Pd(PPh₃)₄ (20 mg, 0.017 mmol) and the resulting mixture was heated at 120° C. for 12 hrs. After that time, the reaction mixture was cooled to room temperature, saturated aqueous KF (5 mL) was added, and the resulting mixture was stirred at room temperature for 1 h. The mixture was filtered through a thin pad of diatomaceous earth and the filter cake was rinsed with EtOAc (3×15 mL). The combined filtrates were extracted with EtOAc (2×25 mL). The combined organic layers were dried over anhydrous Na₂SO₄, filtered and concentrated. The residue was purified by reverse-phase HPLC (Xtimate C18 column, 5 μm, 30×150 mm; 23-53% ACN/H₂O (with 0.05% NH₃ and 10 mM NH₄HCO₃)) to afford, after lyophilization, the title compound as a yellow solid (4 mg, 5%). MS (ESI): Mass calcd. for C₂₀H₁₈F₂N₁₀O₂, 468.4; m/z found, 469.2 [M+H]⁺. ¹H NMR (400 MHz, DMSO-d₆) δ 9.96 (s, 1H), 9.08 (s, 1H), 8.74-8.61 (m, 1H), 8.54-8.44 (m, 1H), 8.41-8.33 (m, 1H), 8.31-8.22 (m, 1H), 7.97-7.83 (m, 1H), 7.62 (s, 1H), 7.46 (s, 1H), 6.73 (s, 1H), 4.13-3.90 (m, 2H), 3.78 (s, 3H), 2.96 (s, 3H).

### Example 121: (R)-3-Hydroxy-1-methyl-3-(1-(6-(2-(pyrazolo[1,5-a]pyridin-3-ylamino)pyrimidin-4-yl)pyridin-2-yl)-1H-1,2,3-triazol-4-yl)pyrrolidin-2-one

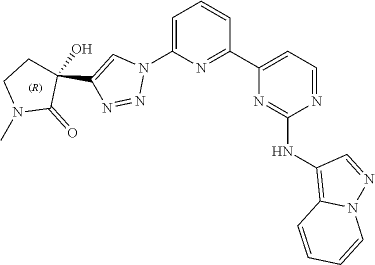

To a solution of (R)-3-(1-(6-(2-chloropyrimidin-4-yl)pyridin-2-yl)-1H-1,2,3-triazol-4-yl)-3-hydroxy-1-methylpyrrolidin-2-one (50 mg, 0.134 mmol, Intermediate 32) in DMSO (1 mL) were added pyrazolo[1,5-a]pyridin-3-amine (53 mg, 0.41 mmol) and DIEA (53 mg, 0.410 mmol) and the resulting mixture was heated at 120° C. for 12 hrs. The mixture was purified by reverse-phase HPLC (Xtimate C18 column, 5 μm, 30×150 mm; 20-50% ACN/H₂O (with 0.05% NH₃ in water and 10 mM NH₄HCO₃)) to afford, after lyophilization, the title compound as a yellow solid (10.7 mg, 16%). MS (ESI): Mass calcd. for C₂₃H₂₀N₁₀O₂, 468.5; m/z found, 469.2 [M+H]⁺. ¹H NMR (400 MHz, DMSO-d₆) δ 9.73 (s, 1H), 8.95 (s, 1H), 8.68 (d, J=5.2 Hz, 1H), 8.59 (d, J=6.8 Hz, 1H), 8.52-8.29 (m, 3H), 8.29-8.24 (m, 1H), 7.88-7.79 (m, 2H), 7.18-7.10 (m, 1H), 6.90-6.80 (m, 1H), 6.34 (s, 1H), 3.49 (m, 2H), 2.83 (s, 3H), 2.82-2.74 (m, 1H), 2.32-2.24 (m, 1H).

### Example 122: (R)-3-(1-(6-(2-((1-(2,2-Difluoroethyl)-3-methoxy-1H-pyrazol-4-yl)amino)pyrimidin-4-yl)pyridin-2-yl)-1H-1,2,3-triazol-4-yl)-3-hydroxy-1-methylpyrrolidin-2-one

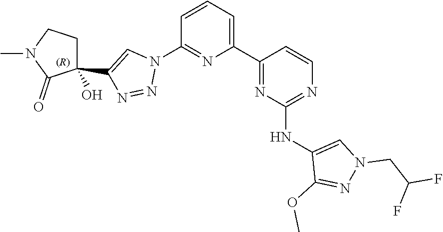

The title compound (66 mg, 42%) was prepared using conditions analogous to those described in Example 120 using (R)-3-(1-(6-bromopyridin-2-yl)-1H-1,2,3-triazol-4-yl)-3-hydroxy-1-methylpyrrolidin-2-one (Intermediate 51) in place of (*R)-3-(1-(6-bromopyridin-2-yl)-1H-1,2,3-triazol-4-yl)-4,4-difluoro-3-hydroxy-1-methylpyrrolidin-2-one and N-(1-(2,2-difluoroethyl)-3-methoxy-1H-pyrazol-4-yl)-4-(tributylstannyl)pyrimidin-2-amine (Intermediate 66) in place of N-(1-methyl-1H-pyrazol-3-yl)-4-(tributylstannyl)pyrimidin-2-amine. MS (ESI): Mass calcd. for C₂₂H₂₂F₂N₁₀O₃, 512.5; m/z found, 513.3 [M+H]⁺. ¹H NMR (400 MHz, DMSO-d₆) δ 8.94 (s, 1H), 8.86-8.67 (m, 1H), 8.61 (d, J=4.8 Hz, 1H), 8.47-8.36 (m, 1H), 8.34-8.22 (m, 2H), 7.94 (s, 1H), 7.76 (d, J=5.2 Hz, 1H), 6.50-6.17 (m, 2H), 4.59-4.41 (m, 2H), 3.85 (s, 3H), 3.54-3.44 (m, 2H), 2.83 (s, 3H), 2.81-2.73 (m, 1H), 2.32-2.23 (m, 1H).

### Example 123: (R)-3-(3-(6-(2-((2-(3-Amino-1H-pyrazol-1-yl)ethyl)amino)pyrimidin-4-yl)pyridin-2-yl)isoxazol-5-yl)-3-hydroxy-1-methylpyrrolidin-2-one

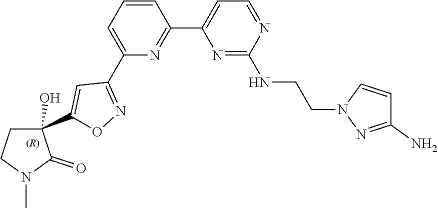

A mixture of (R)-3-hydroxy-1-methyl-3-(3-(6-(2-(methylsulfonyl)pyrimidin-4-yl)pyridin-2-yl)isoxazol-5-yl)pyrrolidin-2-one (50 mg, 120 mol, Intermediate 13), tert-butyl (2-(3-amino-1H-pyrazol-1-yl)ethyl)carbamate (171 mg, 602 mol, Intermediate 71) and TFA (17.9 μL, 241 mol) in DMSO (2 mL) was subjected to microwave irradiation at 150° C. for 2 h. The reaction mixture was cooled to room temperature and concentrated. The residue was purified sequentially by silica gel chromatography (0-5% MeOH/DCM) and then reverse-phase HPLC (Phenomenex C18 column, 3 μm, 30×75 mm; 12-42% ACN/H₂O (with 0.05% NH₃ and 10 mM NH₄HCO₃)) to afford the title compound as a white solid (9.1 mg, 20%). MS (ESI): Mass calcd. for C₂₂H₂₃N₉O₃, 461.5; m/z found, 462.2 [M+H]⁺. ¹H NMR (400 MHz, DMSO-d₆) δ 8.57-8.42 (m, 2H), 8.21-8.09 (m, 2H), 7.66 (d, J=4.8 Hz, 1H), 7.38-7.36 (m, 1H), 7.32 (d, J=2.0 Hz, 1H), 7.15 (s, 1H), 5.36 (d, J=2.0 Hz, 1H), 4.57 (s, 2H), 4.08 (s, 2H), 3.70-3.69 (m, 2H), 3.52-3.43 (m, 2H), 2.85 (s, 3H), 2.65-2.60 (m, 1H), 2.34-2.25 (m, 1H).

### Example 124: (R)-3-(3-(6-(2-((1-Cyclopropyl-1H-pyrazol-4-yl)amino)pyrimidin-4-yl)pyridin-2-yl)isoxazol-5-yl)-3-hydroxy-1-methylpyrrolidin-2-one

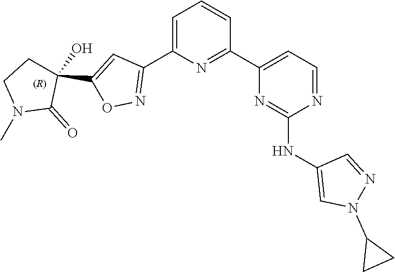

A mixture of (R)-3-hydroxy-1-methyl-3-(3-(6-(2-(methylsulfonyl)pyrimidin-4-yl)pyridin-2-yl)isoxazol-5-yl)pyrrolidin-2-one (55 mg, 0.13 mmol, Intermediate 13), 1-cyclopropyl-1H-pyrazol-4-amine (49 mg, 0.4 mmol) and TsOH (25 mg, 0.13 mmol) in 1,4-dioxane (5 mL) was heated at 110° C. for 12 hrs. After that time, the reaction mixture was concentrated and the residue purified by reverse-phase HPLC (Phenomenex C18 column, 3 μm, 30×75 mm; 35-65% ACN/H₂O (with 0.05% aqueous NH₃)) to afford the title compound as a yellow solid (6.3 mg, 10%). MS (ESI): Mass calcd. for C₂₃H₂₂N₈O₃, 458.5; m/z found, 459.2 [M+H]⁺. ¹H NMR (400 MHz, DMSO-d₆) δ 9.63 (s, 1H), 8.66-8.61 (m, 1H), 8.50-8.44 (m, 1H), 8.26-8.12 (m, 2H), 8.02 (s, 1H), 7.77 (d, J=5.2 Hz, 1H), 7.60 (s, 1H), 7.17 (s, 1H), 6.77 (s, 1H), 3.74 (s, 1H), 3.59-3.41 (m, 2H), 2.85 (s, 3H), 2.63-2.58 (m, 1H), 2.32-2.25 (m, 1H), 1.06-0.94 (m, 4H).

### Example 125: (R)-3-(3-(6-(2-((1-(2-Aminoethyl)-1H-pyrazol-4-yl)amino)pyrimidin-4-yl)pyridin-2-yl)isoxazol-5-yl)-3-hydroxy-1-methylpyrrolidin-2-one

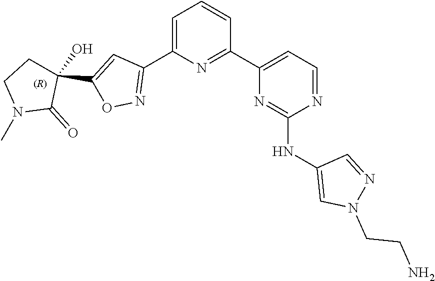

Step A. tert-Butyl (R)-(2-(4-((4-(6-(5-(3-hydroxy-1-methyl-2-oxopyrrolidin-3-yl)isoxazol-3-yl)pyridin-2-yl)pyrimidin-2-yl)amino)-1H-pyrazol-1-yl)ethyl)carbamate. A mixture of (R)-3-Hydroxy-1-methyl-3-(3-(6-(2-(methylsulfonyl)pyrimidin-4-yl)pyridin-2-yl)isoxazol-5-yl)pyrrolidin-2-one (50 mg, 0.12 mmol, Intermediate 13), tert-butyl (2-(4-amino-1H-pyrazol-1-yl)ethyl)carbamate (82 mg, 0.36 mmol) and TsOH (34 mg, 0.18 mmol) in anhydrous 1,4-dioxane (5 mL) was heated at 110° C. for 12 hrs. The reaction mixture was concentrated and purified by reverse-phase HPLC (Phenomenex C18 column, 3 μm, 30×75 mm; 28-58% ACN/H₂O (with 0.05% aqueous NH₃)) to afford the title compound (40 mg, 59%). MS (ESI⁺): m/z=562.3.

Step B. (R)-3-(3-(6-(2-((1-(2-Aminoethyl)-1H-pyrazol-4-yl)amino)pyrimidin-4-yl)pyridin-2-yl)isoxazol-5-yl)-3-hydroxy-1-methylpyrrolidin-2-one. A mixture of tert-butyl (R)-(2-(4-((4-(6-(5-(3-hydroxy-1-methyl-2-oxopyrrolidin-3-yl)isoxazol-3-yl)pyridin-2-yl)pyrimidin-2-yl)amino)-1H-pyrazol-1-yl)ethyl)carbamate (37 mg, 0.066 mmol) and HCl in 1,4-dioxane (2 mL, 4 M) was placed in a sealed tube and stirred at room temperature for 1 h. The solution was concentrated and purified by reverse-phase HPLC (Phenomenex C18 column, 5 μm, 30×150 mm; 18-48% ACN/H₂O (with 0.05% aqueous NH₃)) to afford the title compound as a yellow solid (9.7 mg, 32%). MS (ESI): Mass calcd. for C₂₂H₂₃N₉O₃, 461.5; m/z found, 462.2 [M+H]⁺. ¹H NMR (400 MHz, DMSO-d₆) δ 9.70-9.61 (m, 1H), 8.64 (d, J=4.8 Hz, 1H), 8.49 (s, 1H), 8.25-8.14 (m, 2H), 8.07-7.98 (m, 1H), 7.77 (d, J=5.2 Hz, 1H), 7.63 (s, 1H), 7.17 (s, 1H), 6.81 (s, 1H), 4.17-4.03 (m, 2H), 3.54-3.44 (m, 2H), 2.97-2.89 (m, 2H), 2.85 (s, 3H), 2.65-2.58 (m, 1H), 2.31-2.25 (m, 1H).

### Example 126: (R)-3-(3-(6-(2-((1-(2-(Dimethylamino)ethyl)-1H-pyrazol-3-yl)amino)pyrimidin-4-yl)pyridin-2-yl)isoxazol-5-yl)-3-hydroxy-1-methylpyrrolidin-2-one

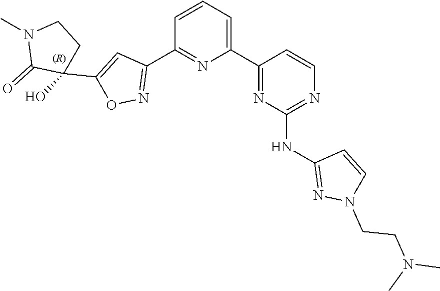

A mixture of (R)-3-(3-(6-(2-chloropyrimidin-4-yl)pyridin-2-yl)isoxazol-5-yl)-3-hydroxy-1-methylpyrrolidin-2-one (150 mg, 0.403 mmol, Intermediate 31), 1-(2-(dimethylamino)ethyl)-1H-pyrazol-3-amine (52.5 mg, 0.340 mmol) and Cs₂CO₃ (413 mg, 1.27 mmol) in 1,4-dioxane (5 mL) was purged with Ar for 5 min. Then, BrettPhos Pd G3 (60 mg, 0.066 mmol) and BrettPhos (37.5 mg, 0.0700 mmol) were added and the resulting mixture was purged with Ar for 5 min and then heated at 90° C. for 16 hrs. The mixture was cooled to room temperature, poured into water (20 mL) and extracted with EtOAc (3×20 mL). The combined organic layers were washed with brine (20 mL), dried over anhydrous Na₂SO₄, filtered and concentrated. The residue was purified twice by reverse-phase HPLC (Xtimate C18 column, 5 μm, 30×150 mm; 27-57% ACN/H₂O (with 0.05% NH₃ in water and 10 mM NH₄HCO₃)) followed by (Boston Prime C18 column, 5 μm, 30×150 mm; 30-60% ACN/H₂O (with 0.05% NH₃ in water and 10 mM NH₄HCO₃)) to afford the title compound as a white solid (22 mg, 12%). MS (ESI): Mass calcd. for C₂₄H₂₇N₉O₃, 489.5; m/z found, 490.3 [M+H]⁺. ¹H NMR (400 MHz, DMSO-d₆) δ 9.94 (s, 1H), 8.64 (d, J=5.2 Hz, 1H), 8.47 (d, J=7.6 Hz, 1H), 8.24-8.13 (m, 2H), 7.82 (d, J=5.2 Hz, 1H), 7.70-7.65 (m, 1H), 7.17 (s, 1H), 6.94-6.65 (m, 2H), 4.12 (t, J=6.4 Hz, 2H), 3.52-3.43 (m, 2H), 2.85 (s, 3H), 2.67 (t, J=6.8 Hz, 2H), 2.63-2.56 (m, 1H), 2.34-2.25 (m, 1H), 2.20 (s, 6H).

### Example 127: (R)-3-Hydroxy-3-(3-(6-(2-(imidazo[1,2-b]pyridazin-8-ylamino)pyrimidin-4-yl)pyridin-2-yl)isoxazol-5-yl)-1-methylpyrrolidin-2-one

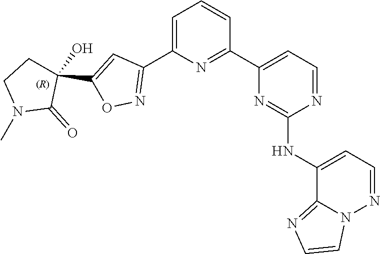

The title compound (26 mg, 24%) was prepared using conditions analogous to those described in Example 126 using imidazo[1,2-b]pyridazin-8-amine in place of 1-(2-(dimethylamino)ethyl)-1H-pyrazol-3-amine. MS (ESI): Mass calcd. for C₂₃H₁₉N₉O₃, 469.5; m/z found, 470.2 [M+H]⁺. ¹H NMR (500 MHz, DMSO-d₆) δ 9.27 (br s, 1H), 8.96-8.92 (m, 1H), 8.68-8.63 (m, 1H), 8.49-8.45 (m, 1H), 8.28-8.15 (m, 5H), 7.75-7.71 (m, 1H), 7.23 (s, 1H), 6.81 (s, 1H), 3.53-3.44 (m, 2H), 2.86 (s, 3H), 2.67-2.63 (m, 1H), 2.34-2.27 (m, 1H).

### Example 128: (R)-3-(3-(6-(2-((2,3-Dimethylpyridin-4-yl)amino)pyrimidin-4-yl)pyridin-2-yl)isoxazol-5-yl)-3-hydroxy-1-methylpyrrolidin-2-one

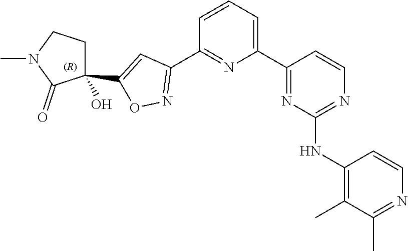

The title compound (15 mg, 12%) was prepared using conditions analogous to those described in Example 126 using 2,3-dimethylpyridin-4-amine in place of 1-(2-(dimethylamino)ethyl)-1H-pyrazol-3-amine. MS (ESI): Mass calcd. for C₂₄H₂₃N₇O₃, 457.5; m/z found, 458.2 [M+H]⁺. ¹H NMR (400 MHz, DMSO-d₆) δ 9.21 (s, 1H), 8.72 (d, J=5.0 Hz, 1H), 8.44-8.35 (m, 1H), 8.21 (d, J=5.5 Hz, 1H), 8.19-8.14 (m, 2H), 7.94 (d, J=5.0 Hz, 1H), 7.74 (d, J=5.6 Hz, 1H), 7.18 (s, 1H), 6.91-6.61 (m, 1H), 3.52-3.47 (m, 2H), 2.85 (s, 3H), 2.65-2.59 (m, 1H), 2.48 (s, 3H), 2.32-2.25 (m, 1H), 2.23 (s, 3H).

### Example 129: (R)-3-Hydroxy-3-(3-(6-(2-((1-(2-hydroxyethyl)-3-methyl-1H-pyrazol-5-yl)amino)pyrimidin-4-yl)pyridin-2-yl)isoxazol-5-yl)-1-methylpyrrolidin-2-one

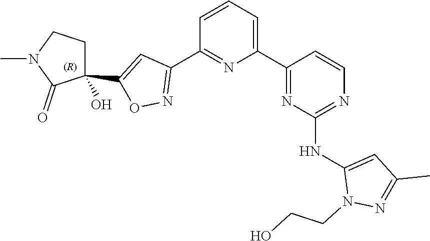

The title compound (20 mg, 15%) was prepared using conditions analogous to those described in Example 126 using 2-(5-amino-3-methyl-1H-pyrazol-1-yl)ethanol in place of 1-(2-(dimethylamino)ethyl)-1H-pyrazol-3-amine. MS (ESI): Mass calcd. for C₂₃H₂₄N₈O₄, 476.5; m/z found, 477.2 [M+H]⁺. ¹H NMR (400 MHz, DMSO-d₆) δ 9.50 (s, 1H), 8.67 (d, J=5.0 Hz, 1H), 8.43-8.34 (m, 1H), 8.26-8.10 (m, 2H), 7.91 (d, J=5.1 Hz, 1H), 7.18 (s, 1H), 6.79 (s, 1H), 6.21 (s, 1H), 5.26-5.03 (m, 1H), 4.11-3.97 (m, 2H), 3.79-3.66 (m, 2H), 3.55-3.42 (m, 2H), 2.85 (s, 3H), 2.65-2.60 (m, 1H), 2.32-2.25 (m, 1H), 2.17 (s, 3H).

### Example 130: (R)-3-(3-(6-(2-((6-(2-(Dimethylamino)ethoxy)pyridazin-3-yl)amino)pyrimidin-4-yl)pyridin-2-yl)isoxazol-5-yl)-3-hydroxy-1-methylpyrrolidin-2-one

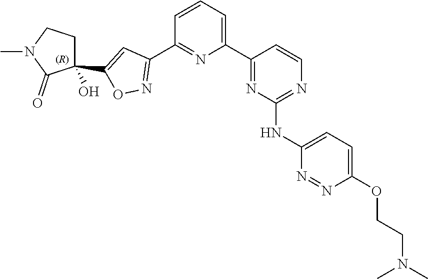

The title compound was prepared using conditions analogous to those described in Example 126 using 6-(2-(dimethylamino)ethoxy)pyridazin-3-amine in place of 1-(2-(dimethylamino)ethyl)-1H-pyrazol-3-amine. The compound was further purified by SFC (DAICEL CHIRALPAK AS, 10 μm, 30×250 mm, isocratic elution: 45% EtOH (0.1% of 25% NH₃ in water), 55% CO₂) to provide the title compound as a white solid (4.3 mg, 4.6%). MS (ESI): Mass calcd. for C₂₅H₂₇N₉O₄, 517.5; m/z found, 518.2 [M+H]⁺. ¹H NMR (400 MHz, DMSO-d₆) δ 10.42 (s, 1H), 8.74 (d, J=5.2 Hz, 1H), 8.50-8.39 (m, 2H), 8.26-8.14 (m, 2H), 7.98 (d, J=5.2 Hz, 1H), 7.32-7.24 (m, 1H), 7.18 (s, 1H), 6.78 (s, 1H), 4.55-4.39 (m, 2H), 3.53-3.45 (m, 2H), 2.85 (s, 3H), 2.71-2.67 (m, 2H), 2.64-2.58 (m, 1H), 2.31-2.26 (m, 1H), 2.23 (s, 6H).

### Example 131: (R)-3-Hydroxy-1-methyl-3-(3-(6-(2-((5-(5-methyl-1H-1,2,4-triazol-1-yl)pyridin-2-yl)amino)pyrimidin-4-yl)pyridin-2-yl)isoxazol-5-yl)pyrrolidin-2-one

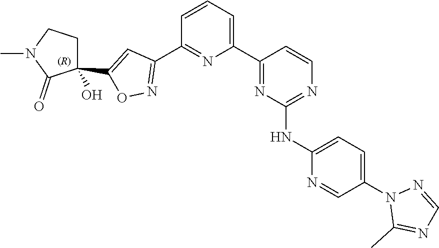

The title compound (17 mg, 11%) was prepared using conditions analogous to those described in Example 126 using 5-(5-methyl-1H-1,2,4-triazol-1-yl)pyridin-2-amine in place of 1-(2-(dimethylamino)ethyl)-1H-pyrazol-3-amine. MS (ESI): Mass calcd. for C₂₅H₂₂N₁₀O₃, 510.5; m/z found, 511.2 [M+H]⁺. ¹H NMR (400 MHz, DMSO-d₆) δ 10.48 (s, 1H), 8.86-8.79 (m, 1H), 8.61-8.53 (m, 3H), 8.27-8.17 (m, 2H), 8.12-8.03 (m, 3H), 7.21 (s, 1H), 6.79 (s, 1H), 3.55-3.41 (m, 2H), 2.85 (s, 3H), 2.66-2.58 (m, 1H), 2.53 (s, 3H), 2.32-2.26 (m, 1H).

### Example 132: (*R)-3-Hydroxy-1-methyl-3-(3-(6-(2-((1-methyl-5-(trifluoromethyl)-1H-pyrazol-4-yl)amino)pyrimidin-4-yl)pyridin-2-yl)isoxazol-5-yl)pyrrolidin-2-one

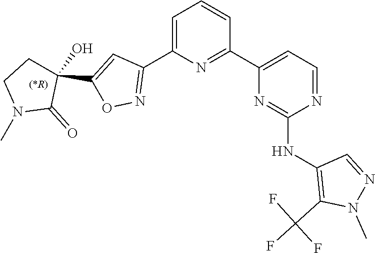

A solution of 1-methyl-5-(trifluoromethyl)-1H-pyrazol-4-amine (170 mg, 0.843 mmol) in THF (4 mL) was cooled to −72° C. and then (R)-3-hydroxy-1-methyl-3-(3-(6-(2-(methylsulfonyl)pyrimidin-4-yl)pyridin-2-yl)isoxazol-5-yl)pyrrolidin-2-one (170 mg, 0.409 mmol, Intermediate 13) and LiHMDS (1.9 mL, 1.9 mmol, 1 M in THF) were added. The resulting mixture was stirred at −72° C. for 1 h and then room temperature for 16 hrs. After that time, the mixture was diluted with EtOAc (50 mL) and washed with water (2×30 mL). The organic layer was dried over anhydrous Na₂SO₄, filtered and concentrated to give a yellow oil. The oil was purified by reverse-phase HPLC (Phenomenex C18 column, 3 μm, 40×80 mm; 45-75% ACN/H₂O (with 0.05% aqueous NH₃ and 10 mM NH₄HCO₃)) to afford the title compound as a yellow solid (35 mg, 16%). MS (ESI): Mass calcd. for C₂₂H₁₉F₃N₈O₃, 500.4; m/z found, 501.2 [M+H]⁺. ¹H NMR (400 MHz, DMSO-d₆) δ 9.14 (s, 1H), 8.66-8.56 (m, 1H), 8.38-8.29 (m, 1H), 8.20-8.10 (m, 2H), 7.87-7.79 (m, 2H), 7.17 (s, 1H), 6.80 (s, 1H), 3.98 (s, 3H), 3.53-3.42 (m, 2H), 2.85 (s, 3H), 2.69-2.57 (m, 1H), 2.35-2.21 (m, 1H).

### Example 133: (1R,4R,5S)-4-(3-(6-(2-((5,6-Dihydro-4H-pyrrolo[1,2-b]pyrazol-3-yl)amino)pyrimidin-4-yl)pyridin-2-yl)isoxazol-5-yl)-4-hydroxy-2-methyl-2-azabicyclo[3.1.0]hexan-3-one

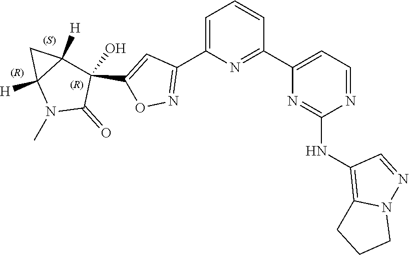

A mixture of (1R,4R,5S)-4-(3-(6-bromopyridin-2-yl)isoxazol-5-yl)-4-hydroxy-2-methyl-2-azabicyclo[3.1.0]hexan-3-one (277 mg, 0.79 mmol, Intermediate 33) and N-(4-(tributylstannyl)pyrimidin-2-yl)-5,6-dihydro-4H-pyrrolo[1,2-b]pyrazol-3-amine (582 mg, 1.19 mmol, Intermediate 67) in 1,4-dioxane (10 mL) was purged with N₂ three times and then Pd(dppf)Cl₂ (58 mg, 0.079 mmol) was added. The mixture was purged with N₂ three times, then heated at 110° C. for 16 hrs. The reaction mixture was allowed to slowly cool to room temperature, then was diluted with water (5 mL) and extracted with EtOAc (3×10 mL). The combined organic layers were washed with brine (10 mL), dried over anhydrous Na₂SO₄, filtered and concentrated. The residue was dissolved in DMF (2 mL), Biotage® MP-TMT (3 mL) was added and the resulting mixture was stirred at room temperature for 16 hrs. The mixture was filtered and the filtrate concentrated to provide a black oil. The oil was purified sequentially by reverse-phase HPLC (Xtimate C18 column, 5 μm, 30×150 mm; 23-53% ACN/H₂O (with 0.2% formic acid)) and then SFC (DAICEL CHIRALPAK AS, 10 μm, 30×250 mm, isocratic elution: 45% EtOH (0.1% of 25% aqueous NH₃), 55% CO₂) to provide the title compound as a yellow solid (24 mg, 20%). MS (ESI): Mass calcd. for C₂₄H₂₂N₈O₃, 470.5; m/z found, 471.2 [M+H]⁺. ¹H NMR (400 MHz, DMSO-d₆) δ 9.23 (s, 1H), 8.62-8.54 (m, 1H), 8.44-8.34 (m, 1H), 8.23-8.11 (m, 2H), 7.76-7.59 (m, 2H), 7.16 (s, 1H), 6.67 (br.s., 1H), 4.06 (t, J=7.2 Hz, 2H), 3.48-3.43 (m, 2H), 2.91-2.82 (m, 5H), 2.56-2.53 (m, 1H), 2.14-2.02 (m, 1H), 1.00-0.90 (m, 1H), 0.85-0.78 (m, 1H).

### Example 134: (R)-3-(5-(6-(2-((5,6-Dihydro-4H-pyrrolo[1,2-b]pyrazol-3-yl)amino)pyrimidin-4-yl)pyridin-2-yl)isoxazol-3-yl)-3-hydroxy-1-methylpyrrolidin-2-one

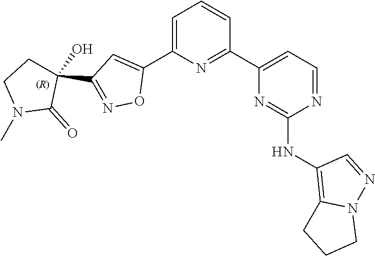

A flask containing (R)-3-(5-(6-bromopyridin-2-yl)isoxazol-3-yl)-3-hydroxy-1-methylpyrrolidin-2-one (200 mg, 0.59 mmol, Intermediate 52) and N-(4-(tributylstannyl)pyrimidin-2-yl)-5,6-dihydro-4H-pyrrolo[1,2-b]pyrazol-3-amine (435 mg, 0.89 mmol, Intermediate 67) in 1,4-dioxane (20 mL) was evacuated and refilled with N₂ three times, then Pd(PPh₃)₂Cl₂ (42 mg, 0.059 mmol) was added. The mixture was evacuated and refilled with N₂ three times, then heated at 100° C. for 16 hrs. The mixture was cooled to room temperature, water (10 mL) was added and the mixture extracted with EtOAc (3×15 mL). The combined organic layers were washed with brine (10 mL), dried over anhydrous Na₂SO₄, filtered and concentrated. The residue was purified by reverse-phase HPLC (Phenomenex C18 column, 3 μm, 40×80 mm; 30-60% ACN/H₂O (with 0.05% aqueous NH₃ and 10 mM NH₄HCO₃)) to afford a yellow solid, which was triturated with MeCN (10 mL). The yellow precipitate that formed was filtered and dried under high vacuum followed by purification by SFC (DAICEL CHIRALPAK AD, 10 μm, 30×250 mm, isocratic elution: 55% EtOH (0.1% of 25% aqueous NH₃), 45% CO₂) to provide the title compound as a yellow solid (92 mg, 26%). MS (ESI): Mass calcd. for C₂₃H₂₂N₈O₃, 458.5; m/z found, 459.2 [M+H]⁺. ¹H NMR (400 MHz, DMSO-d₆) δ 9.24 (br. s., 1H), 8.61-8.54 (m, 1H), 8.40-8.33 (m, 1H), 8.25-8.17 (m, 1H), 8.12-8.05 (m, 1H), 7.74-7.57 (m, 2H), 7.32 (s, 1H), 6.56 (s, 1H), 4.10-4.02 (m, 2H), 3.52-3.36 (m, 2H), 2.92-2.86 (m, 2H), 2.81 (s, 3H), 2.75-2.67 (m, 1H), 2.58-2.51 (m, 2H), 2.31-2.21 (m, 1H).

### Example 135: (R)-3-Hydroxy-3-(5-(6-(2-((2-(methoxymethyl)-2H-1,2,3-triazol-4-yl)amino)pyrimidin-4-yl)pyridin-2-yl)isoxazol-3-yl)-1-methylpyrrolidin-2-one

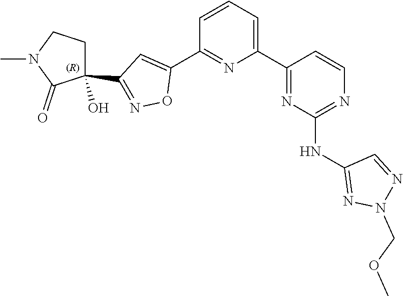

The title compound (24 mg, 20%) was prepared using conditions analogous to those described in Example 134 using N-(2-(methoxymethyl)-2H-1,2,3-triazol-4-yl)-4-(tributylstannyl)pyrimidin-2-amine (Intermediate 69) in place of N-(4-(tributylstannyl)pyrimidin-2-yl)-5,6-dihydro-4H-pyrrolo[1,2-b]pyrazol-3-amine. MS (ESI): Mass calcd. for C₂₁H₂₁N₉O₄, 463.5; m/z found, 464.2 [M+H]⁺. ¹H NMR (400 MHz, DMSO-d₆) δ 10.61 (br s, 1H), 8.74 (d, J=4.8 Hz, 1H), 8.49 (d, J=7.6 Hz, 1H), 8.30 (s, 1H), 8.26 (t, J=7.6 Hz, 1H), 8.14 (d, J=7.6 Hz, 1H), 7.91 (d, J=4.8 Hz, 1H), 7.36 (s, 1H), 6.58 (s, 1H), 5.57 (s, 2H), 3.51-3.39 (m, 2H), 3.32 (s, 3H), 2.82 (s, 3H), 2.76-2.67 (m, 1H), 2.31-2.22 (m, 1H).

### Example 136: (R)-3-Hydroxy-3-(1-(4-methoxy-6-(2-((1-methyl-1H-pyrazol-4-yl)amino)pyrimidin-4-yl)pyridin-2-yl)-1H-1,2,3-triazol-4-yl)-1-methylpyrrolidin-2-one

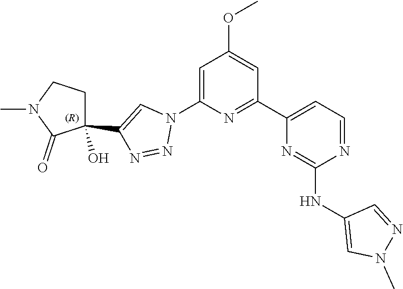

To a mixture of (R)-3-(1-(6-bromo-4-methoxypyridin-2-yl)-1H-1,2,3-triazol-4-yl)-3-hydroxy-1-methylpyrrolidin-2-one (50 mg, 0.136 mmol, Intermediate 79), N-(1-methyl-1H-pyrazol-4-yl)-4-(tributylstannyl)pyrimidin-2-amine (70 mg, 0.151 mmol, Intermediate 76) and TEA (30 mg, 0.30 mmol) in toluene (2 mL) was added Pd(PPh₃)₄ (16 mg, 0.014 mmol) and the resulting mixture was heated at 110° C. for 12 hrs. The mixture was cooled to room temperature, filtered, and the filter cake was collected. The solids were purified by reverse-phase HPLC (Xtimate C18 column, 5 μm, 25×150 mm; 15-45% ACN/H₂O (with 0.05% aqueous NH₃)) to afford, after lyophilization, the title compound as a yellow solid (3.8 mg, 6%). MS (ESI): Mass calcd. for C₂₁H₂₂N₁₀O₃, 462.5; m/z found, 463.3 [M+H]⁺. ¹H NMR (400 MHz, DMSO-d₆) δ 9.66 (s, 1H), 8.90 (s, 1H), 8.64 (d, J=5.2 Hz, 1H), 7.98 (s, 2H), 7.75-7.66 (m, 2H), 7.58 (s, 1H), 6.32 (s, 1H), 4.09 (s, 3H), 3.84 (s, 3H), 3.49 (t, J=6.8 Hz, 2H), 2.86-2.72 (m, 4H), 2.34-2.22 (m, 1H).

### Example 137: (R)-3-(1-(4-(Difluoromethyl)-6-(2-((1-methyl-1H-pyrazol-4-yl)amino)pyrimidin-4-yl)pyridin-2-yl)-1H-1,2,3-triazol-4-yl)-3-hydroxy-1-methylpyrrolidin-2-one

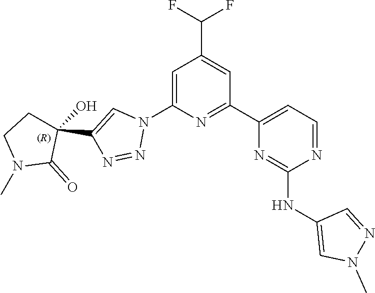

To a mixture of (R)-3-(1-(6-chloro-4-(difluoromethyl)pyridin-2-yl)-1H-1,2,3-triazol-4-yl)-3-hydroxy-1-methylpyrrolidin-2-one (90 mg, 0.26 mmol, Intermediate 77), N-(1-methyl-1H-pyrazol-4-yl)-4-(tributylstannyl)pyrimidin-2-amine (135 mg, 0.291 mmol, Intermediate 76) and TEA (60 mg, 0.59 mmol) in toluene (2 mL) was added Pd(PPh₃)₄ (30 mg, 0.026 mmol). The resulting mixture was heated at 110° C. for 12 hrs. The reaction was cooled to room temperature, filtered and the filter cake was collected. The solids were purified by reverse-phase HPLC (Xtimate C18 column, 5 μm, 25×150 mm; 15-45% ACN/H₂O (with 0.05% aqueous NH₃)) to afford, after lyophilization, the title compound as a yellow solid (2.6 mg, 2%). MS (ESI): Mass calcd. for C₂₁H₂₀F₂N₁₀O₂, 482.5; m/z found, 483.3 [M+H]⁺. ¹H NMR (400 MHz, DMSO-d₆) δ 9.75 (s, 1H), 9.00 (s, 1H), 8.70 (d, J=5.2 Hz, 1H), 8.59 (s, 1H), 8.41 (s, 1H), 7.95 (s, 1H), 7.81 (d, J=5.2 Hz, 1H), 7.64-7.51 (m, 1H), 7.44-7.23 (m, 1H), 6.35 (s, 1H), 3.84 (s, 3H), 3.50 (t, J=6.4 Hz, 2H), 2.85-2.73 (m, 4H), 2.35-2.24 (m, 1H).

### Example 138: (R)-3-Hydroxy-3-(1-(4-(methoxymethyl)-6-(2-((1-methyl-1H-pyrazol-4-yl)amino)pyrimidin-4-yl)pyridin-2-yl)-1H-1,2,3-triazol-4-yl)-1-methylpyrrolidin-2-one

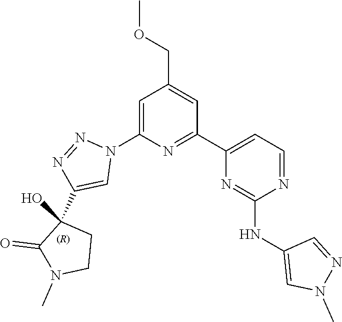

To a mixture of (R)-3-(1-(6-chloro-4-(methoxymethyl)pyridin-2-yl)-1H-1,2,3-triazol-4-yl)-3-hydroxy-1-methylpyrrolidin-2-one (130 mg, 0.385 mmol, Intermediate 78), N-(1-methyl-1H-pyrazol-4-yl)-4-(tributylstannyl)pyrimidin-2-amine (200 mg, 0.431 mmol, Intermediate 76) and TEA (90 mg, 0.89 mmol) in toluene (3 mL) was added Pd(PPh₃)₄ (45 mg, 0.039 mmol) and the resulting mixture was heated at 90° C. for 12 hrs. The reaction mixture was cooled to room temperature, filtered and the filter cake was collected. The solids were purified by reverse-phase HPLC (Xtimate C18 column, 5 μm, 25×150 mm; 12-42% ACN/H₂O (with 0.05% aqueous NH₃)) to afford, after lyophilization, the title compound as a yellow solid (9.2 mg, 2%). MS (ESI): Mass calcd. for C₂₂H₂₄N₁₀O₃, 476.5; m/z found, 477.2 [M+H]⁺. ¹H NMR (400 MHz, DMSO-d₆) δ 9.71 (s, 1H), 8.94 (s, 1H), 8.66 (d, J=5.0 Hz, 1H), 8.43 (s, 1H), 8.18 (s, 1H), 7.98 (s, 1H), 7.76 (d, J=5.0 Hz, 1H), 7.57 (s, 1H), 6.33 (s, 1H), 4.76 (s, 2H), 3.85 (s, 3H), 3.51-3.48 (m, 2H), 3.47 (s, 3H), 2.83 (s, 3H), 2.80-2.73 (m, 1H), 2.32-2.24 (m, 1H), 1.23 (s, 1H).

### Example 139: (R)-3-Hydroxy-1-methyl-3-(3-(6-(2-((1-(1-methylpiperidin-4-yl)-1H-pyrazol-4-yl)amino)pyrimidin-4-yl)pyridin-2-yl)isoxazol-5-yl)pyrrolidin-2-one

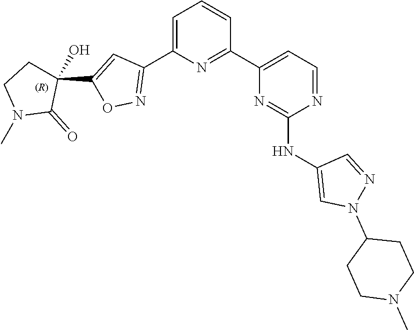

The title compound was prepared using conditions analogous to those described in Example 204 Step A using 1-(1-methylpiperidin-4-yl)-1H-pyrazol-4-amine in place of tert-butyl (2-(3-amino-1H-pyrazol-1-yl)ethyl)carbamate. The residue was purified by reverse-phase HPLC (Xtimate C18 column, 5 μm, 30×150 mm; 25-55% ACN/H₂O (with 0.225% formic acid)) to afford the title compound as a yellow solid (4.6 mg, 17%). MS (ESI): Mass calcd. for C₂₆H₂₉N₉O₃, 515.6; m/z found, 516.0 [M+H]⁺. ¹H NMR (400 MHz, DMSO-d₆) δ 9.62 (s, 1H), 8.63 (d, J=4.8 Hz, 1H), 8.47 (d, J=7.6 Hz, 1H), 8.25-8.19 (m, 1H), 8.18-8.15 (m, 1H), 7.99 (s, 1H), 7.76 (d, J=5.2 Hz, 1H), 7.72-7.61 (m, 1H), 7.17 (s, 1H), 6.78 (s, 1H), 4.18-4.06 (m, 1H), 3.52-3.45 (m, 1H), 2.89-2.86 (m, 1H), 2.85 (s, 3H), 2.69-2.59 (m, 1H), 2.36-2.24 (m, 2H), 2.21 (s, 3H), 2.09-1.90 (m, 7H).

### Example 140 and Example 141: (R)-3-Hydroxy-1-methyl-3-(3-(6-(2-((1-((S)-1-(oxetan-3-yl)pyrrolidin-3-yl)-1H-pyrazol-4-yl)amino)pyrimidin-4-yl)pyridin-2-yl)isoxazol-5-yl)pyrrolidin-2-one and (R)-3-hydroxy-1-methyl-3-(3-(6-(2-((1-((R)-1-(oxetan-3-yl)pyrrolidin-3-yl)-1H-pyrazol-4-yl)amino)pyrimidin-4-yl)pyridin-2-yl)isoxazol-5-yl)pyrrolidin-2-one

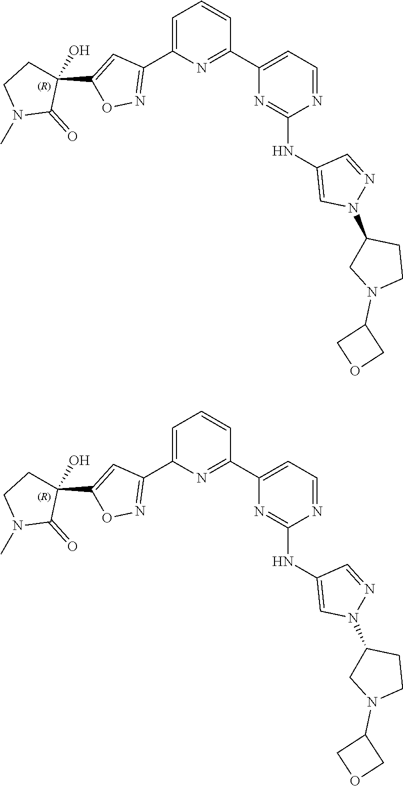

The title compounds were prepared using conditions analogous to those described in Example 204 Step A using 1-(1-(oxetan-3-yl)pyrrolidin-3-yl)-1H-pyrazol-4-amine (Intermediate 73) in place of tert-butyl (2-(3-amino-1H-pyrazol-1-yl)ethyl)carbamate and purifying the residue by preparative TLC (9% MeOH/DCM) to provide a mixture of diastereomers. The diastereomers were separated by SFC (DAICEL CHIRALPAK AD, m, 30×250 mm, isocratic elution: 55% EtOH (0.05% NH₃·H₂O), 45% CO₂). The first-eluting diastereomer was further purified by reverse-phase HPLC (Boston Prime C18 column, 5 μm, 30×150 mm; 25-55% ACN/H₂O (with 0.05% NH₃)) to afford Example 140 as a yellow solid (2.7 mg, 5%). The second-eluting diastereomer was further purified by reverse-phase HPLC (Boston Prime C18 column, 5 μm, 30×150 mm; 25-55% ACN/H₂O (with 0.05% NH₃)) to afford Example 141 as a yellow solid (3.9 mg, 6%). Example 140: MS (ESI): Mass calcd. for C₂₇H₂₉N₉O₄, 543.6; m/z found, 544.3 [M+H]⁺. ¹H NMR (400 MHz, DMSO-d₆) δ 9.67 (s, 1H), 8.64 (d, J=4.8 Hz, 1H), 8.49 (d, J=6.8 Hz, 1H), 8.25-8.20 (m, 1H), 8.19-8.15 (m, 1H), 8.14-8.09 (m, 1H), 7.78 (d, J=5.2 Hz, 1H), 7.64 (s, 1H), 7.17 (s, 1H), 6.78 (s, 1H), 5.01-4.88 (m, 1H), 4.63-4.57 (m, 2H), 4.54-4.47 (m, 2H), 3.71-3.64 (m, 1H), 3.49-3.43 (m, 1H), 2.95-2.90 (m, 1H), 2.85 (s, 3H), 2.82-2.76 (m, 2H), 2.69-2.58 (m, 4H), 2.35-2.31 (m, 1H), 2.16-2.03 (m, 1H). Example 141: MS (ESI): Mass calcd. for C₂₇H₂₉N₉O₄, 543.6; m/z found, 544.3 [M+H]⁺. ¹H NMR (400 MHz, DMSO-d₆) δ 9.67 (s, 1H), 8.64 (d, J=5.2 Hz, 1H), 8.49 (d, J=7.2 Hz, 1H), 8.26-8.19 (m, 1H), 8.19-8.15 (m, 1H), 8.12 (s, 1H), 7.78 (d, J=5.2 Hz, 1H), 7.64 (s, 1H), 7.17 (s, 1H), 6.78 (s, 1H), 4.95 (s, 1H), 4.63-4.57 (m, 2H), 4.54-4.46 (m, 2H), 3.70-3.65 (m, 1H), 3.49-3.43 (m, 1H), 2.95-2.90 (m, 1H), 2.85 (s, 3H), 2.81-2.76 (m, 2H), 2.69-2.59 (m, 4H), 2.34-2.30 (m, 1H), 2.14-2.05 (m, 1H).

### Example 142 and Example 143: (R)-3-Hydroxy-1-methyl-3-(1-(6-(2-(((R)-1-(pyrimidin-2-yl)ethyl)amino)pyrimidin-4-yl)pyridin-2-yl)-1H-1,2,3-triazol-4-yl)pyrrolidin-2-one and (R)-3-hydroxy-1-methyl-3-(1-(6-(2-(((S)-1-(pyrimidin-2-yl)ethyl)amino)pyrimidin-4-yl)pyridin-2-yl)-1H-1,2,3-triazol-4-yl)pyrrolidin-2-one

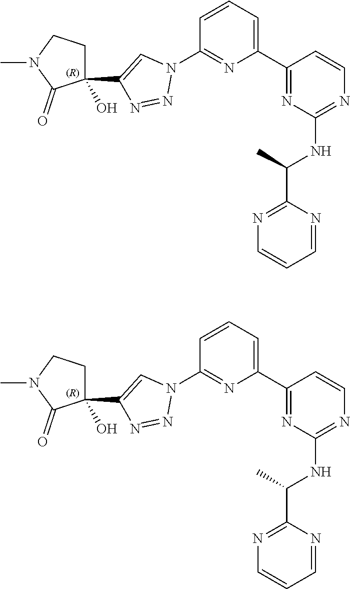

To a solution of (R)-3-hydroxy-1-methyl-3-(1-(6-(2-(methylsulfonyl)pyrimidin-4-yl)pyridin-2-yl)-1H-1,2,3-triazol-4-yl)pyrrolidin-2-one (100 mg, 0.241 mmol, Intermediate 75) and 1-(pyrimidin-2-yl)ethanamine hydrochloride (192 mg, 1.20 mmol) in NMP (2 mL) was added DIEA (0.80 mL, 4.84 mmol). The resulting mixture was heated at 130° C. for 16 hrs. The mixture was cooled to room temperature, diluted with EtOAc (10 mL), washed sequentially with water (10 mL) and brine (10 mL), dried over anhydrous Na₂SO₄, filtered and concentrated. The residue was purified by reverse-phase HPLC (Xtimate C18 column, 5 μm, 30×150 mm; 20-50% ACN/H₂O (with 0.05% NH₃ and 10 mM NH₄HCO₃)) to provide a mixture of diastereomers as a white solid. The diastereomers were separated by chiral SFC (DAICEL CHIRALPAK AD, 10 m, 30×250 mm, isocratic elution: 50% IPA (0.1% NH₃·H₂O), 50% CO₂) to provide Example 142 as the first-eluting diastereomer (31 mg, 34%) and Example 143 as the second-eluting diastereomer (17 mg, 19%). Example 142: MS (ESI): Mass calcd. for C₂₂H₂₂N₁₀O₂, 458.5; m/z found, 459.2 [M+H]⁺. ¹H NMR (400 MHz, DMSO-d₆) δ 8.89 (br. s., 1H), 8.78 (d, J=4.8 Hz, 2H), 8.61-8.41 (m, 1H), 8.40-7.94 (m, 3H), 7.92-7.66 (m, 1H), 7.60 (d, J=4.8 Hz, 1H), 7.41-7.27 (m, 1H), 6.57-6.13 (m, 1H), 5.31-5.15 (m, 1H), 3.55-3.45 (m, 2H), 2.82 (s, 3H), 2.80-2.72 (m, 1H), 2.31-2.22 (m, 1H), 1.58 (d, J=7.2 Hz, 3H). Example 143: MS (ESI): Mass calcd. for C₂₂H₂₂N₁₀O₂, 458.5; m/z found, 459.2 [M+H]⁺. ¹H NMR (400 MHz, DMSO-d₆) δ 8.89 (s, 1H), 8.78 (d, J=4.8 Hz, 2H), 8.61-8.37 (m, 1H), 8.35-7.99 (m, 3H), 7.94-7.67 (m, 1H), 7.60 (d, J=4.8 Hz, 1H), 7.41-7.29 (m, 1H), 6.67-5.93 (m, 1H), 5.30-5.18 (m, 1H), 3.54-3.45 (m, 2H), 2.82 (s, 3H), 2.80-2.73 (m, 1H), 2.31-2.23 (m, 1H), 1.58 (d, J=7.2 Hz, 3H).

### Example 144: (R)-3-(1-(6-(2-((1-(2,2-Difluoroethyl)-3-methyl-1H-pyrazol-4-yl)amino)pyrimidin-4-yl)pyridin-2-yl)-1H-1,2,3-triazol-4-yl)-3-hydroxy-1-methylpyrrolidin-2-one

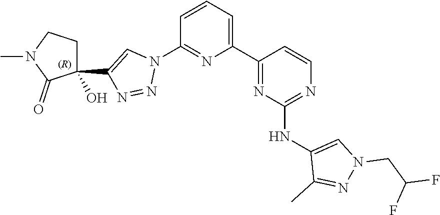

The title compound was prepared using conditions analogous to those described in Example 204 Step A using (R)-3-(1-(6-(2-chloropyrimidin-4-yl)pyridin-2-yl)-1H-1,2,3-triazol-4-yl)-3-hydroxy-1-methylpyrrolidin-2-one (Intermediate 32) in place of (R)-3-(3-(6-(2-chloropyrimidin-4-yl)pyridin-2-yl)isoxazol-5-yl)-3-hydroxy-1-methylpyrrolidin-2-one and 1-(2,2-difluoroethyl)-3-methyl-1H-pyrazol-4-amine in place of tert-butyl (2-(3-amino-1H-pyrazol-1-yl)ethyl)carbamate. The material was further purified by SFC (SAICEL CHIRALPAK AD, 10 μm, 30×250 mm, isocratic elution: 55% IPA (0.1% NH₃·H₂O), 45% CO₂) to afford the title compound as a yellow solid (8 mg, 52%). MS (ESI): Mass calcd. for C₂₂H₂₂F₂N₁₀O₂, 496.5; m/z found, 497.3 [M+H]⁺. ¹H NMR (400 MHz, DMSO-d₆) δ 9.11 (br. s., 1H), 8.94 (s, 1H), 8.65 (d, J=5.2 Hz, 1H), 8.50-8.39 (m, 1H), 8.35-8.22 (m, 2H), 8.08 (br. s., 1H), 7.78 (d, J=5.2 Hz, 1H), 6.53-6.13 (m, 2H), 4.64-4.49 (m, 2H), 3.52-3.45 (m, 2H), 2.83 (s, 3H), 2.81-2.74 (m, 1H), 2.32-2.24 (m, 1H), 2.20 (s, 3H).

### Example 145: (R)-3-Hydroxy-3-(3-(4-methoxy-6-(2-((1-methyl-1H-pyrazol-4-yl)amino)pyrimidin-4-yl)pyridin-2-yl)isoxazol-5-yl)-1-methylpyrrolidin-2-one

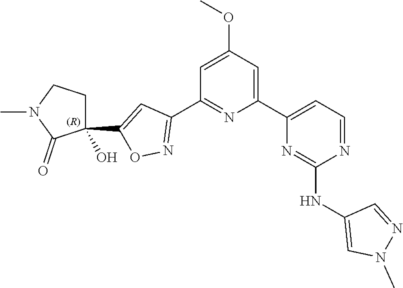

A mixture of (R)-3-hydroxy-3-(3-(4-methoxy-6-(2-(methylsulfonyl)pyrimidin-4-yl)pyridin-2-yl)isoxazol-5-yl)-1-methylpyrrolidin-2-one (100 mg, 0.224 mmol, Intermediate 74), 1-methyl-1H-pyrazol-4-amine (300 mg, 1.76 mmol) and DIEA (800 μL, 4.59 mmol) in DMA (2 mL) was heated at 150° C. for 3 hrs. The mixture was cooled to room temperature gradually then concentrated. The residue was purified by reverse-phase HPLC (Boston Prime C18 column, 5 μm, 30×150 mm; 30-60% ACN/H₂O (with 0.05% NH₃ in water and 10 mM NH₄HCO₃)) to afford the title compound as a white solid (16 mg, 15%). MS (ESI): Mass calcd. for C₂₂H₂₂N₈O₄, 462.5; m/z found, 463.2 [M+H]⁺. ¹H NMR (400 MHz, DMSO-d₆) δ 9.64 (s, 1H), 8.63 (d, J=5.0 Hz, 1H), 8.05-7.93 (m, 2H), 7.74 (d, J=5.0 Hz, 1H), 7.65 (d, J=2.4 Hz, 1H), 7.58 (br s, 1H), 7.15 (s, 1H), 6.77 (s, 1H), 4.05 (s, 3H), 3.83 (s, 3H), 3.53-3.48 (m, 1H), 3.46-3.41 (m, 1H), 2.85 (s, 3H), 2.65-2.59 (m, 1H), 2.32-2.25 (m, 1H).

### Example 146: (R)-3-Hydroxy-3-(3-(4-methoxy-6-(2-((1-methyl-1H-pyrazol-3-yl)amino)pyrimidin-4-yl)pyridin-2-yl)isoxazol-5-yl)-1-methylpyrrolidin-2-one

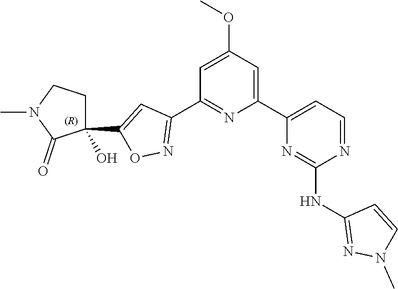

A flask containing (R)-3-(3-(6-bromo-4-methoxypyridin-2-yl)isoxazol-5-yl)-3-hydroxy-1-methylpyrrolidin-2-one (200 mg, 0.543 mmol, Intermediate 74 Step E), N-(1-methyl-1H-pyrazol-3-yl)-4-(tributylstannyl)pyrimidin-2-amine (296 mg, 0.638 mmol, Intermediate 24) and TEA (172 μL, 1.23 mmol) in toluene (3 mL) was evacuated and refilled with Ar three times, then Pd(PPh₃)₄ (68 mg, 0.059 mmol) was added. The resulting mixture was heated at 120° C. for 16 hrs. The flask was gradually cooled to room temperature and then saturated aqueous KF (15 mL) was added over 2 min. The mixture was stirred at room temperature for 2 hrs, then extracted with EtOAc (3×15 mL). The combined organic layers were washed with brine (20 mL), dried over anhydrous Na₂SO₄, filtered and concentrated. The residue was purified by reverse-phase HPLC (Xtimate C18 column, 10 μm, 25×150 mm; 32-62% ACN/H₂O (with 0.05% NH₃ in water and 10 mM NH₄HCO₃)) to afford the title compound as a white solid (19 mg, 7%). MS (ESI): Mass calcd. for C₂₂H₂₂N₈O₄, 462.5; m/z found, 463.2 [M+H]⁺. ¹H NMR (400 MHz, DMSO-d₆) δ 9.89 (s, 1H), 8.63 (d, J=5.0 Hz, 1H), 8.01 (d, J=2.4 Hz, 1H), 7.79 (d, J=5.0 Hz, 1H), 7.71-7.53 (m, 2H), 7.15 (s, 1H), 6.78 (s, 1H), 6.68 (d, J=2.1 Hz, 1H), 4.02 (s, 3H), 3.77 (s, 3H), 3.50-3.48 (m, 2H), 2.85 (s, 3H), 2.62-2.60 (m, 1H), 2.32-2.24 (m, 1H).

### Example 147: (3R)-3-(3-(6-(2-((1-(1H-Pyrazol-3-yl)ethyl)amino)pyrimidin-4-yl)pyridin-2-yl)isoxazol-5-yl)-3-hydroxy-1-methylpyrrolidin-2-one

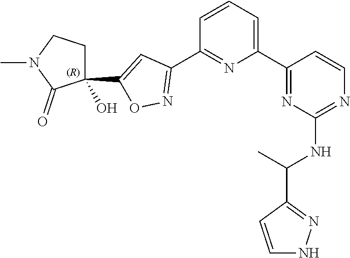

A mixture of (R)-3-hydroxy-1-methyl-3-(3-(6-(2-(methylsulfonyl)pyrimidin-4-yl)pyridin-2-yl)isoxazol-5-yl)pyrrolidin-2-one (150 mg, 0.361 mmol, Intermediate 13), 1-(1H-pyrazol-3-yl)ethanamine hydrochloride (405 mg, 2.74 mmol) and DIEA (1.0 mL, 5.7 mmol) in DMA (4 mL) was sparged with N₂ for 5 min, then heated at 130° C. for 16 hrs. The mixture was cooled to room temperature and then diluted with water (5 mL) and extracted with EtOAc (3×10 mL). The combined organic layers were washed with brine (8 mL), dried over anhydrous Na₂SO₄, filtered and concentrated. The residue was purified by reverse-phase HPLC (Xtimate C18 column, 5 μm, 25×150 mm; 28-58% ACN/H₂O (with 0.05% NH₃ in water and 10 mM NH₄HCO₃)) to afford the title compound as a white solid (27 mg, 17%). MS (ESI): Mass calcd. for C₂₂H₂₂N₈O₃, 446.5; m/z found, 447.2 [M+H]⁺. ¹H NMR (400 MHz, DMSO-d₆) δ 12.76-12.43 (m, 1H), 8.58-8.37 (m, 2H), 8.21-8.09 (m, 2H), 7.70-7.27 (m, 3H), 7.15 (s, 1H), 6.78 (s, 1H), 6.23 (br s, 1H), 5.38 (br s, 1H), 3.53-3.41 (m, 2H), 2.85 (s, 3H), 2.66-2.58 (m, 1H), 2.32-2.24 (m, 1H), 1.55-1.49 (m, 3H).

### Example 148 and Example 149: (R)-3-(3-(6-(2-(((R)-1-(1H-Pyrazol-4-yl)ethyl)amino)pyrimidin-4-yl)pyridin-2-yl)isoxazol-5-yl)-3-hydroxy-1-methylpyrrolidin-2-one and (R)-3-(3-(6-(2-(((S)-1-(1H-pyrazol-4-yl)ethyl)amino)pyrimidin-4-yl)pyridin-2-yl)isoxazol-5-yl)-3-hydroxy-1-methylpyrrolidin-2-one

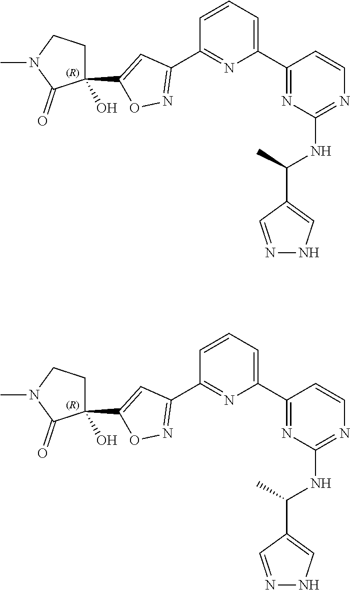

The title compounds were prepared using conditions analogous to those described in Example 147 using 1-(1H-pyrazol-4-yl)ethanamine in place of 1-(1H-pyrazol-3-yl)ethanamine hydrochloride to provide a mixture of diastereomers. The diastereomers were separated by SFC (Phenomenex-Cellulose-2, 10 μm, 30×250 mm, isocratic elution: 40% MeOH (0.1% NH₃·H₂O), 60% CO₂) to provide Example 148 as the first-eluting diastereomer (8 mg, 39%) and Example 149 as the second-eluting diastereomer (8.5 mg, 42%). Example 148: MS (ESI): Mass calcd. for C₂₂H₂₂N₈O₃, 446.5; m/z found, 447.2 [M+H]⁺. ¹H NMR (400 MHz, DMSO-d₆) δ 12.57 (br. s., 1H), 8.55-8.42 (m, 2H), 8.21-8.09 (m, 2H), 7.68-7.39 (m, 4H), 7.15 (s, 1H), 6.78 (br. s., 1H), 5.40-5.17 (m, 1H), 3.54-3.39 (m, 2H), 2.85 (s, 3H), 2.66-2.58 (m, 1H), 2.31-2.24 (m, 1H), 1.50 (d, J=6.8 Hz, 3H). Example 149: MS (ESI): Mass calcd. for C₂₂H₂₂N₈O₃, 446.5; m/z found, 447.2 [M+H]⁺. ¹H NMR (400 MHz, DMSO-d₆) δ 12.58 (br. s., 1H), 8.57-8.39 (m, 2H), 8.20-8.09 (m, 2H), 7.75-7.41 (m, 4H), 7.15 (s, 1H), 6.78 (br. s., 1H), 5.42-5.15 (m, 1H), 3.53-3.40 (m, 2H), 2.85 (s, 3H), 2.66-2.57 (m, 1H), 2.32-2.24 (m, 1H), 1.50 (d, J=6.8 Hz, 3H).

### Example 150 and Example 151: (R)-3-Hydroxy-1-methyl-3-(3-(6-(2-(((R)-1-(3-methyl-1,2,4-oxadiazol-5-yl)ethyl)amino)pyrimidin-4-yl)pyridin-2-yl)isoxazol-5-yl)pyrrolidin-2-one and (R)-3-hydroxy-1-methyl-3-(3-(6-(2-(((S)-1-(3-methyl-1,2,4-oxadiazol-5-yl)ethyl)amino)pyrimidin-4-yl)pyridin-2-yl)isoxazol-5-yl)pyrrolidin-2-one

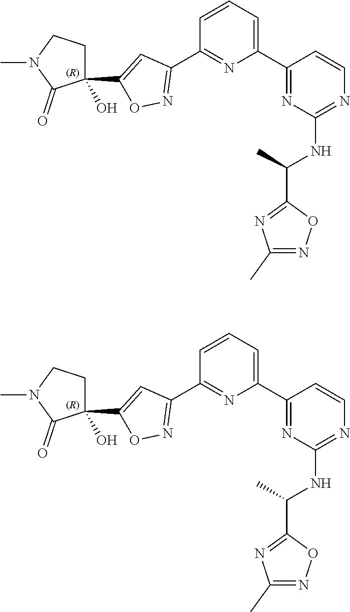

The title compounds were prepared using conditions analogous to those described in Example 147 using (R)-2-amino-N-methyl-N-phenylpropanamide in place of 1-(1H-pyrazol-3-yl)ethanamine hydrochloride to provide a mixture of diastereomers. The diastereomers were separated by SFC (DAICEL CHIRALCEL OJ, 10 μm, 30×250 mm, isocratic elution: 30% EtOH (0.1% NH₃·H₂O), 70% CO₂) to provide Example 150 as the first-eluting diastereomer (4 mg, 9%) and Example 151 as the second-eluting diastereomer (6 mg, 15%). Example 150: MS (ESI): Mass calcd. for C₂₂H₂₂N₈O₄, 462.5; m/z found, 463.2 [M+H]⁺. ¹H NMR (400 MHz, DMSO-d₆) δ 8.55 (br s, 1H), 8.40-8.07 (m, 4H), 7.72 (d, J=5.0 Hz, 1H), 7.15 (s, 1H), 6.78 (br s, 1H), 5.45-5.30 (m, 1H), 3.53-3.40 (m, 2H), 2.85 (s, 3H), 2.65-2.57 (m, 1H), 2.31 (s, 3H), 2.29-2.25 (m, 1H), 1.64 (d, J=7.2 Hz, 3H). Example 151: MS (ESI): Mass calcd. for C₂₂H₂₂N₈O₄, 462.5; m/z found, 463.2 [M+H]⁺. ¹H NMR (400 MHz, DMSO-d₆) δ 8.55 (br s, 1H), 8.35-8.03 (m, 4H), 7.72 (d, J=5.0 Hz, 1H), 7.15 (s, 1H), 6.78 (br s, 1H), 5.49-5.28 (m, 1H), 3.53-3.40 (m, 2H), 2.84 (s, 3H), 2.65-2.57 (m, 1H), 2.30 (s, 3H), 2.29-2.24 (m, 1H), 1.64 (d, J=7.0 Hz, 3H).

### Example 152: (R)-3-Hydroxy-3-(3-(6-(2-(((1 S,2R)-2-hydroxy-1-phenylpropyl)amino)pyrimidin-4-yl)pyridin-2-yl)isoxazol-5-yl)-1-methylpyrrolidin-2-one

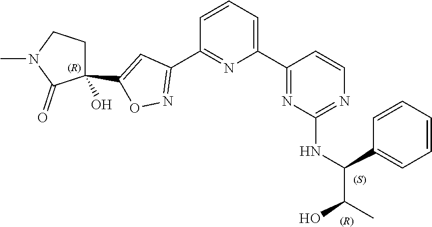

The title compound (28 mg, 48%) was prepared using conditions analogous to those described in Example 147 using (1S,2R)-1-amino-1-phenylpropan-2-ol hydrochloride in place of 1-(1H-pyrazol-3-yl)ethanamine hydrochloride. MS (ESI): Mass calcd. for C₂₆H₂₆N₆O₄, 486.5; m/z found, 487.2 [M+H]⁺. ¹H NMR (400 MHz, DMSO-d₆) δ 8.52-8.35 (m, 2H), 8.21-8.07 (m, 2H), 7.71-7.56 (m, 2H), 7.47 (br s, 2H), 7.33-7.24 (m, 2H), 7.21-7.15 (m, 1H), 7.13 (s, 1H), 6.77 (s, 1H), 5.01-4.93 (m, 1H), 4.80-4.75 (m, 1H), 4.09-4.00 (m, 1H), 3.52-3.41 (m, 2H), 2.84 (s, 3H), 2.65-2.57 (m, 1H), 2.32-2.23 (m, 1H), 1.16-1.08 (m, 3H).

### Example 153: (R)-3-Hydroxy-3-(3-(6-(2-(((S)-1-(imidazo[1,2-a]pyridin-6-yl)ethyl)amino)pyrimidin-4-yl)pyridin-2-yl)isoxazol-5-yl)-1-methylpyrrolidin-2-one

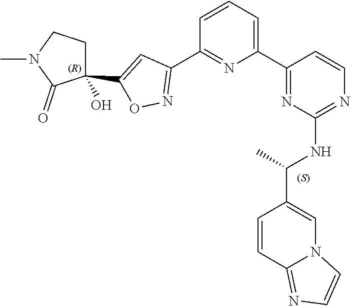

The title compound was prepared using conditions analogous to those described in Example 147 using (S)-1-(Imidazo[1,2-a]pyridin-6-yl)ethanamine hydrochloride in place of 1-(1H-pyrazol-3-yl)ethanamine hydrochloride. The material was further purified by SFC (DAICEL CHIRALCEL OD, 5 μm, 30×250 mm, isocratic elution: 55% EtOH (0.1% NH₃·H₂O), 45% CO₂) to afford the title compound as a colorless solid (14 mg, 69%). MS (ESI): Mass calcd. for C₂₆H₂₄N₈O₃, 496.5; m/z found, 497.2 [M+H]⁺. ¹H NMR (400 MHz, DMSO-d₆) δ 8.67-8.43 (m, 3H), 8.18-8.09 (m, 2H), 8.00-7.91 (m, 2H), 7.66-7.61 (m, 1H), 7.57-7.49 (m, 2H), 7.45-7.36 (m, 1H), 7.13 (s, 1H), 6.77 (s, 1H), 5.25 (br s, 1H), 3.52-3.42 (m, 2H), 2.84 (s, 3H), 2.66-2.56 (m, 1H), 2.32-2.23 (m, 1H), 1.59-1.53 (m, 3H).

### Example 154 and Example 155: (R)-3-Hydroxy-1-methyl-3-(3-(6-(2-(((R)-1-(pyridin-4-yl)ethyl)amino)pyrimidin-4-yl)pyridin-2-yl)isoxazol-5-yl)pyrrolidin-2-one and (R)-3-hydroxy-1-methyl-3-(3-(6-(2-(((S)-1-(pyridin-4-yl)ethyl)amino)pyrimidin-4-yl)pyridin-2-yl)isoxazol-5-yl)pyrrolidin-2-one

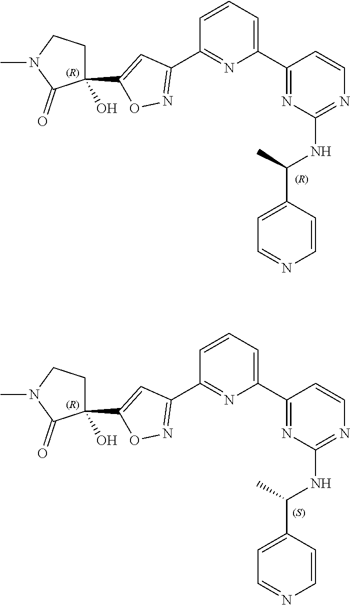

The title compounds were prepared using conditions analogous to those described in Example 147 using (R)-1-(pyridin-4-yl)ethanamine in place of 1-(1H-pyrazol-3-yl)ethanamine hydrochloride to provide a mixture of diastereomers. The diastereomers were separated by SFC (DAICEL CHIRALPAK AD, 10 μm, 30×250 mm, isocratic elution: 55% IPA (0.1% NH₃·H₂O), 45% CO₂) to provide Example 154 as the first-eluting diastereomer (28 mg, 28%) and Example 155 as the second-eluting diastereomer (22 mg, 21%). Example 154: MS (ESI): Mass calcd. for C₂₄H₂₃N₇O₃, 457.5; m/z found, 458.2 [M+H]⁺. ¹H NMR (400 MHz, DMSO-d₆) δ 8.59-8.20 (m, 4H), 8.19-7.92 (m, 3H), 7.66-7.59 (m, 1H), 7.53-7.35 (m, 2H), 7.12 (s, 1H), 6.93-6.47 (m, 1H), 5.27-5.10 (m, 1H), 3.54-3.39 (m, 2H), 2.84 (s, 3H), 2.65-2.57 (m, 1H), 2.31-2.23 (m, 1H), 1.50 (d, J=7.2 Hz, 3H). Example 155: MS (ESI): Mass calcd. for C₂₄H₂₃N₇O₃, 457.5; m/z found, 458.2 [M+H]⁺. ¹H NMR (400 MHz, DMSO-d₆) δ 8.57-8.44 (m, 3H), 8.43-7.99 (m, 4H), 7.63 (d, J=5.2 Hz, 1H), 7.55-7.37 (m, 2H), 7.13 (s, 1H), 6.78 (br. s., 1H), 5.28-5.10 (m, 1H), 3.53-3.40 (m, 2H), 2.84 (s, 3H), 2.65-2.57 (m, 1H), 2.32-2.24 (m, 1H), 1.50 (d, J=7.2 Hz, 3H).

### Example 156 and Example 157: (R)-3-Hydroxy-1-methyl-3-(3-(6-(2-(((R)-1-(3-methylpyridin-2-yl)ethyl)amino)pyrimidin-4-yl)pyridin-2-yl)isoxazol-5-yl)pyrrolidin-2-one and (R)-3-hydroxy-1-methyl-3-(3-(6-(2-(((S)-1-(3-methylpyridin-2-yl)ethyl)amino)pyrimidin-4-yl)pyridin-2-yl)isoxazol-5-yl)pyrrolidin-2-one

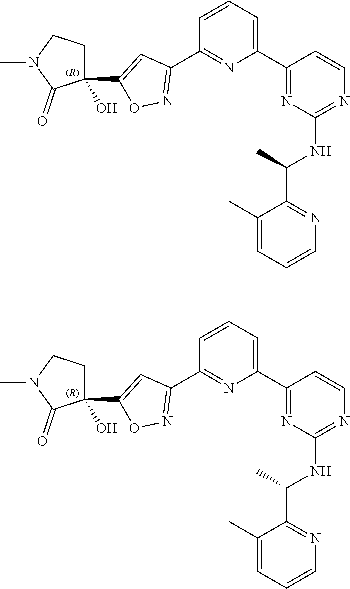

The title compounds were prepared using conditions analogous to those described in Example 147 using 1-(3-methylpyridin-2-yl)ethanamine dihydrochloride in place of 1-(1H-pyrazol-3-yl)ethanamine hydrochloride to provide a mixture of diastereomers. The diastereomers were separated by SFC (DAICEL CHIRALPAK AD, 10 μm, 30×250 mm, isocratic elution: 55% MeOH (0.1% NH₃·H₂O), 45% CO₂) to provide Example 156 as the first-eluting diastereomer (30 mg, 32%) and Example 157 as the second-eluting diastereomer (30 mg, 32%). Example 156: MS (ESI): Mass calcd. for C₂₅H₂₅N₇O₃, 471.5; m/z found, 472.2 [M+H]⁺. ¹H NMR (400 MHz, DMSO-d₆) δ 8.59-8.26 (m, 3H), 8.19-8.09 (m, 2H), 7.67-7.55 (m, 2H), 7.52-7.35 (m, 1H), 7.24-7.10 (m, 2H), 6.78 (s, 1H), 5.53-5.38 (m, 1H), 3.54-3.40 (m, 2H), 2.85 (s, 3H), 2.70-2.57 (m, 1H), 2.47 (br s, 3H), 2.35-2.24 (m, 1H), 1.51-1.43 (m, 3H). Example 157: MS (ESI): Mass calcd. for C₂₅H₂₅N₇O₃, 471.5; m/z found, 472.2 [M+H]⁺. ¹H NMR (400 MHz, DMSO-d₆) δ 8.57-8.27 (m, 3H), 8.20-8.09 (m, 2H), 7.68-7.55 (m, 2H), 7.53-7.33 (m, 1H), 7.25-7.10 (m, 2H), 6.78 (s, 1H), 5.56-5.37 (m, 1H), 3.53-3.41 (m, 2H), 2.85 (s, 3H), 2.69-2.56 (m, 1H), 2.47 (br s, 3H), 2.35-2.24 (m, 1H), 1.51-1.43 (m, 3H).

### Example 158: (R)-3-Hydroxy-3-(3-(6-(2-(((1 S,2S)-2-hydroxy-1-phenylpropyl)amino)pyrimidin-4-yl)pyridin-2-yl)isoxazol-5-yl)-1-methylpyrrolidin-2-one

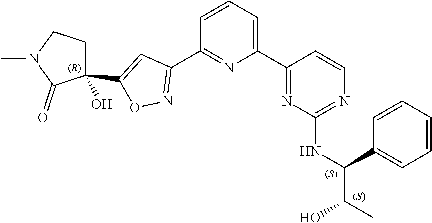

The title compound (58 mg, 50%) was prepared using conditions analogous to those described in Example 147 using (1S,2S)-1-amino-1-phenylpropan-2-ol in place of 1-(1H-pyrazol-3-yl)ethanamine hydrochloride. MS (ESI): Mass calcd. for C₂₆H₂₆N₆O₄, 486.5; m/z found, 487.2 [M+H]⁺. ¹H NMR (400 MHz, DMSO-d₆) δ 8.51-8.34 (m, 2H), 8.21-8.07 (m, 2H), 7.71-7.55 (m, 2H), 7.47 (br s, 2H), 7.33-7.25 (m, 2H), 7.22-7.15 (m, 1H), 7.13 (s, 1H), 6.76 (s, 1H), 5.03-4.91 (m, 1H), 4.77 (d, J=5.2 Hz, 1H), 4.12-3.98 (m, 1H), 3.54-3.39 (m, 2H), 2.85 (s, 3H), 2.66-2.56 (m, 1H), 2.32-2.24 (m, 1H), 1.12 (br d, J=6.0 Hz, 3H).

### Example 159: (R)-3-Hydroxy-3-(3-(6-(2-(((1R,2R)-2-hydroxy-1-phenylpropyl)amino)pyrimidin-4-yl)pyridin-2-yl)isoxazol-5-yl)-1-methylpyrrolidin-2-one

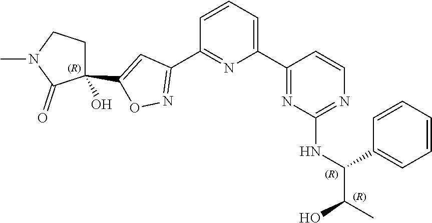

The title compound (25 mg, 21%) was prepared using conditions analogous to those described in Example 147 using (1R,2R)-1-amino-1-phenylpropan-2-ol in place of 1-(1H-pyrazol-3-yl)ethanamine hydrochloride. MS (ESI): Mass calcd. for C₂₆H₂₆N₆O₄, 486.5; m/z found, 487.2 [M+H]⁺. ¹H NMR (400 MHz, DMSO-d₆) δ 8.51-8.34 (m, 2H), 8.19-8.08 (m, 2H), 7.74-7.56 (m, 2H), 7.48 (br s, 2H), 7.33-7.25 (m, 2H), 7.22-7.15 (m, 1H), 7.13 (s, 1H), 6.77 (s, 1H), 5.03-4.92 (m, 1H), 4.78 (d, J=5.2 Hz, 1H), 4.11-3.99 (m, 1H), 3.54-3.39 (m, 2H), 2.84 (s, 3H), 2.67-2.56 (m, 1H), 2.35-2.23 (m, 1H), 1.12 (d, J=6.0 Hz, 3H).

### Example 160 and Example 161: (R)-3-Hydroxy-3-(3-(6-(2-(((R)-1-(imidazo[1,2-a]pyridin-3-yl)ethyl)amino)pyrimidin-4-yl)pyridin-2-yl)isoxazol-5-yl)-1-methylpyrrolidin-2-one and (R)-3-hydroxy-3-(3-(6-(2-(((S)-1-(imidazo[1,2-a]pyridin-3-yl)ethyl)amino)pyrimidin-4-yl)pyridin-2-yl)isoxazol-5-yl)-1-methylpyrrolidin-2-one

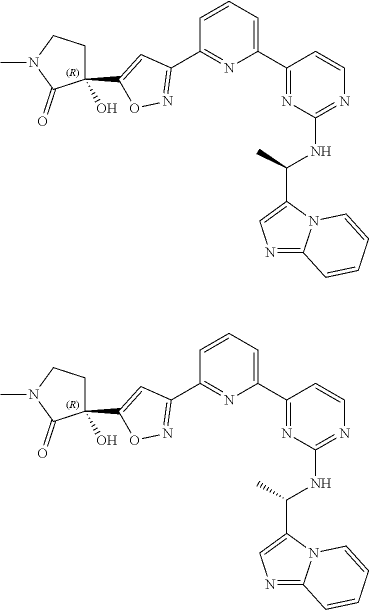

The title compounds were prepared using conditions analogous to those described in Example 147 using (R)-1-(imidazo[1,2-a]pyridin-3-yl)ethanamine dihydrochloride in place of 1-(1H-pyrazol-3-yl)ethanamine hydrochloride to provide a mixture of diastereomers. The diastereomers were separated by SFC (DAICEL CHIRALCEL OJ, 10 μm, 30×250 mm, isocratic elution: 35% EtOH (0.1% NH₃·H₂O), 65% CO₂) to provide Example 160 as the first-eluting diastereomer (2 mg, 10%) and Example 161 as the second-eluting diastereomer (5 mg, 25%). Example 160: MS (ESI): Mass calcd. for C₂₆H₂₄N₈O₃, 496.5; m/z found, 497.2 [M+H]⁺. ¹H NMR (400 MHz, DMSO-d₆) δ 8.59-8.30 (m, 3H), 8.15-8.11 (m, 2H), 7.87-7.77 (m, 1H), 7.68 (d, J=2.4 Hz, 1H), 7.62 (s, 1H), 7.57 (d, J=4.8 Hz, 1H), 7.25-7.18 (m, 1H), 7.15 (s, 1H), 6.91 (t, J=6.8 Hz, 1H), 6.77 (s, 1H), 5.88-5.66 (m, 1H), 3.51-3.42 (m, 2H), 2.85 (s, 3H), 2.66-2.57 (m, 1H), 2.31-2.24 (m, 1H), 1.73 (d, J=6.8 Hz, 3H). Example 161: MS (ESI): Mass calcd. for C₂₆H₂₄N₈O₃, 496.5; m/z found, 497.2 [M+H]⁺. ¹H NMR (400 MHz, DMSO-d₆) δ 8.60-8.30 (m, 3H), 8.15-8.11 (m, 2H), 7.87-7.78 (m, 1H), 7.68 (d, J=5.2 Hz, 1H), 7.62 (s, 1H), 7.57 (d, J=9.2 Hz, 1H), 7.25-7.18 (m, 1H), 7.14 (s, 1H), 6.91 (t, J=6.8 Hz, 1H), 6.77 (s, 1H), 5.85-5.70 (m, 1H), 3.51-3.41 (m, 2H), 2.84 (s, 3H), 2.65-2.57 (m, 1H), 2.31-2.25 (m, 1H), 1.72 (d, J=6.8 Hz, 3H).

### Example 162 and Example 163: (R)-3-Hydroxy-1-methyl-3-(3-(6-(2-(((R)-2-methyl-1-(1-methyl-1H-pyrazol-4-yl)propyl)amino)pyrimidin-4-yl)pyridin-2-yl)isoxazol-5-yl)pyrrolidin-2-one and (R)-3-hydroxy-1-methyl-3-(3-(6-(2-(((S)-2-methyl-1-(1-methyl-1H-pyrazol-4-yl)propyl)amino)pyrimidin-4-yl)pyridin-2-yl)isoxazol-5-yl)pyrrolidin-2-one

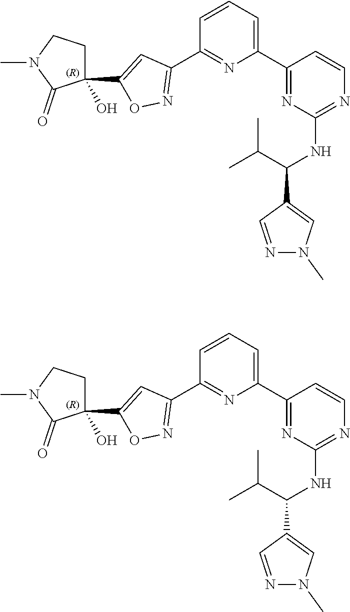

The title compounds were prepared using conditions analogous to those described in Example 147 using 2-methyl-1-(1-methyl-1H-pyrazol-4-yl)propan-1-amine in place of 1-(1H-pyrazol-3-yl)ethanamine hydrochloride to provide a mixture of diastereomers. The diastereomers were separated by SFC (DAICEL CHIRALCEL OJ, 10 μm, 30×250 mm, isocratic elution: 30% EtOH (0.1% NH₃·H₂O), 70% CO₂) to provide Example 162 as the first-eluting diastereomer (25 mg, 30%) and Example 163 as the second-eluting diastereomer (25 mg, 30%). Example 162: MS (ESI): Mass calcd. for C₂₅H₂₈N₈O₃, 488.5; m/z found, 489.2 [M+H]⁺. ¹H NMR (400 MHz, DMSO-d₆) δ 8.48 (d, J=5.2 Hz, 2H), 8.22-8.10 (m, 2H), 7.67-7.50 (m, 3H), 7.44 (s, 1H), 7.15 (s, 1H), 6.79 (s, 1H), 5.04-4.89 (m, 1H), 3.77 (s, 3H), 3.55-3.40 (m, 2H), 2.85 (s, 3H), 2.66-2.57 (m, 1H), 2.33-2.25 (m, 1H), 2.15-1.98 (m, 1H), 0.96 (d, J=6.4 Hz, 3H), 0.86 (d, J=6.8 Hz, 3H). Example 163: MS (ESI): Mass calcd. for C₂₅H₂₈N₈O₃, 488.5; m/z found, 489.3 [M+H]⁺. ¹H NMR (400 MHz, DMSO-d₆) δ 8.48 (d, J=5.2 Hz, 2H), 8.22-8.10 (m, 2H), 7.66-7.49 (m, 3H), 7.43 (s, 1H), 7.14 (s, 1H), 6.79 (s, 1H), 5.04-4.89 (m, 1H), 3.77 (s, 3H), 3.53-3.41 (m, 2H), 2.85 (s, 3H), 2.66-2.58 (m, 1H), 2.33-2.24 (m, 1H), 2.14-2.00 (m, 1H), 0.96 (d, J=6.4 Hz, 3H), 0.86 (d, J=6.8 Hz, 3H).

### Example 164 and Example 165: (R)-3-Hydroxy-3-(3-(6-(2-(((R)-1-(imidazo[1,2-a]pyridin-7-yl)ethyl)amino)pyrimidin-4-yl)pyridin-2-yl)isoxazol-5-yl)-1-methylpyrrolidin-2-one and (R)-3-hydroxy-3-(3-(6-(2-(((S)-1-(imidazo[1,2-a]pyridin-7-yl)ethyl)amino)pyrimidin-4-yl)pyridin-2-yl)isoxazol-5-yl)-1-methylpyrrolidin-2-one

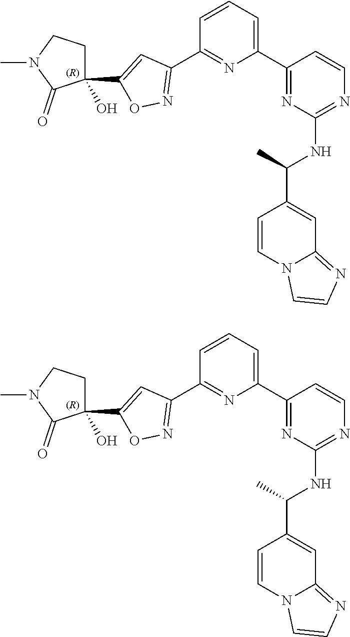

The title compounds were prepared using conditions analogous to those described in Example 147 using 1-(imidazo[1,2-a]pyridin-7-yl)ethanamine in place of 1-(1H-pyrazol-3-yl)ethanamine hydrochloride to provide a mixture of diastereomers. The diastereomers were separated by SFC (DAICEL CHIRALCEL OJ, 10 μm, 30×250 mm, isocratic elution: 35% EtOH (0.1% of 25% aqueous NH₃), 65% CO₂) to provide Example 164 as the first-eluting diastereomer (8.5 mg, 19%) and Example 165 as the second-eluting diastereomer (9 mg, 20%). Example 164: MS (ESI): Mass calcd. for C₂₆H₂₄N₈O₃, 496.5; m/z found, 497.2 [M+H]⁺. ¹H NMR (400 MHz, DMSO-d₆) δ 8.55-8.46 (m, 2H), 8.45-8.40 (m, 1H), 8.18-8.08 (m, 2H), 8.06-7.97 (m, 1H), 7.84 (s, 1H), 7.65-7.61 (m, 1H), 7.60-7.47 (m, 2H), 7.13 (s, 1H), 7.09-6.98 (m, 1H), 6.80 (br s, 1H), 5.27 (br s, 1H), 3.53-3.40 (m, 2H), 2.84 (s, 3H), 2.65-2.57 (m, 1H), 2.33-2.24 (m, 1H), 1.54 (d, J=7.2 Hz, 3H). Example 165: MS (ESI): Mass calcd. for C₂₆H₂₄N₈O₃, 496.5; m/z found, 497.2 [M+H]⁺. ¹H NMR (400 MHz, DMSO-d₆) δ 8.52-8.46 (m, 2H), 8.46-8.41 (m, 1H), 8.18-8.08 (m, 2H), 8.06-7.97 (m, 1H), 7.84 (s, 1H), 7.65-7.61 (m, 1H), 7.60-7.46 (m, 2H), 7.13 (s, 1H), 7.09-6.98 (m, 1H), 6.79 (s, 1H), 5.27 (br s, 1H), 3.53-3.40 (m, 2H), 2.84 (s, 3H), 2.66-2.56 (m, 1H), 2.32-2.24 (m, 1H), 1.54 (d, J=7.2 Hz, 3H).

### Example 166: (*R)-3-Hydroxy-1-methyl-3-(1-methyl-5-(6-(2-((1-methyl-1H-pyrazol-3-yl)amino)pyrimidin-4-yl)pyridin-2-yl)-1H-pyrazol-3-yl)pyrrolidin-2-one

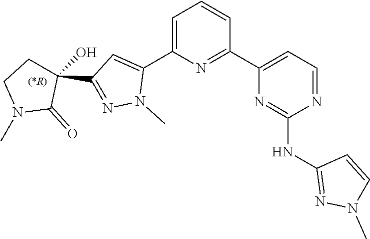 [SMILES: CN1CC[C@@](O)(c2cc(-c3cccc(-c4ccnc(Nc5ccn(C)n5)n4)n3)n(C)n2)C1=O]

To a mixture of 3-(5-(6-chloropyridin-2-yl)-1-methyl-1H-pyrazol-3-yl)-3-hydroxy-1-methylpyrrolidin-2-one (500 mg, 1.63 mmol, Intermediate 65) and N-(1-methyl-1H-pyrazol-3-yl)-4-(tributylstannyl)pyrimidin-2-amine (760 mg, 1.64 mmol, Intermediate 24) in 1,4-dioxane (10 mL) was added Pd(dppf)Cl₂ (240 mg, 0.328 mmol). The mixture was sparged with N₂ three times, then heated at 100° C. for 16 hrs. The reaction mixture was allowed to slowly cool to room temperature, then was diluted with water (8 mL) and extracted with EtOAc (3×15 mL). The combined organic layers were washed with brine (10 mL), dried over anhydrous Na₂SO₄, filtered and concentrated. The residue was purified by reverse-phase HPLC (Xtimate C18 column, 5 μm, 30×150 mm; 25-55% ACN/H₂O (with 0.05% NH₃ and 10 mM NH₄HCO₃)) to provide a mixture of the (R) and (S) enantiomers of 3-hydroxy-1-methyl-3-(1-methyl-5-(6-(2-((1-methyl-1H-pyrazol-3-yl)amino)pyrimidin-4-yl)pyridin-2-yl)-1H-pyrazol-3-yl)pyrrolidin-2-one as a white solid. The enantiomers were separated by chiral SFC (DAICEL CHIRALCEL OJ, 10 μm, 30×250 mm, isocratic elution: 65% EtOH (0.1% of 25% aqueous NH₃), 35% CO₂) to provide Example 166 as the first-eluting enantiomer which was designated as the (*R) enantiomer (10 mg, 20%). MS (ESI): Mass calcd. for C₂₂H₂₃N₉O₂, 445.5; m/z found, 446.2 [M+H]⁺. ¹H NMR (400 MHz, DMSO-d₆) δ 9.43 (br s, 1H), 8.64-8.56 (m, 1H), 8.37-8.29 (m, 1H), 8.13-8.04 (m, 1H), 7.91-7.84 (m, 1H), 7.70-7.64 (m, 1H), 7.59-7.51 (m, 1H), 6.90 (s, 1H), 6.70-6.63 (m, 1H), 5.61 (br s, 1H), 4.21 (s, 3H), 3.77 (s, 3H), 3.41-3.34 (m, 2H), 2.78 (s, 3H), 2.75-2.67 (m, 1H), 2.21-2.12 (m, 1H).

### Example 167: (R)-3-(3-(6-(2-(((R)-1-(Azetidin-1-yl)-1-oxopropan-2-yl)amino)pyrimidin-4-yl)pyridin-2-yl)isoxazol-5-yl)-3-hydroxy-1-methylpyrrolidin-2-one

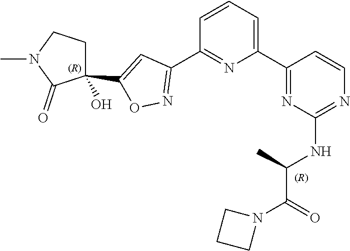

A mixture of (R)-3-hydroxy-1-methyl-3-(3-(6-(2-(methylsulfonyl)pyrimidin-4-yl)pyridin-2-yl)isoxazol-5-yl)pyrrolidin-2-one (50 mg, 0.12 mmol, Intermediate 13), (R)-2-amino-1-(azetidin-1-yl)propan-1-one (155 mg, 0.121 mmol) and DIEA (0.31 mL, 1.8 mmol) in DMA (1 mL) was sparged with N₂ for 5 min, then heated at 130° C. for 16 hrs. The mixture was cooled to room temperature and then diluted with water (2 mL) and extracted with EtOAc (3×4 mL). The combined organic layers were washed with brine (2 mL), dried over anhydrous Na₂SO₄, filtered and concentrated. The residue was purified sequentially by reverse-phase HPLC (Xtimate C18 column, 5 μm, 30×150 mm; 17-47% ACN/H₂O (with 0.05% NH₃ in water and 10 mM NH₄HCO₃)) and SFC (DAICEL CHIRALCEL OD-H, 5 μm, 30×250 mm, isocratic elution: 65% EtOH (0.1% of aqueous NH₃), 35% CO₂) to afford the title compound as a colorless solid (6.3 mg, 11%). MS (ESI): Mass calcd. for C₂₃H₂₅N₇O₄, 463.5; m/z found, 464.3 [M+H]⁺. ¹H NMR (400 MHz, DMSO-d₆) δ 8.54-8.47 (m, 1H), 8.45-8.39 (m, 1H), 8.18-8.07 (m, 2H), 7.66-7.62 (m, 1H), 7.09 (s, 1H), 7.03-6.97 (m, 1H), 6.50 (s, 1H), 4.58-4.48 (m, 1H), 4.46-4.31 (m, 1H), 4.26-4.06 (m, 1H), 4.00-3.80 (m, 2H), 3.55-3.38 (m, 2H), 2.85 (s, 3H), 2.66-2.58 (m, 1H), 2.35-2.20 (m, 3H), 1.40-1.32 (m, 3H).

### Example 168: (R)-3-(3-(6-(2-(((S)-1-(Azetidin-1-yl)-1-oxopropan-2-yl)amino)pyrimidin-4-yl)pyridin-2-yl)isoxazol-5-yl)-3-hydroxy-1-methylpyrrolidin-2-one

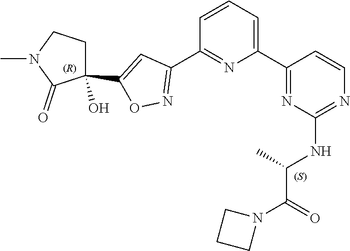

The title compound (10 mg, 12%) was prepared using conditions analogous to those described in Example 167 using (S)-2-amino-1-(azetidin-1-yl)propan-1-one in place of (R)-2-amino-1-(azetidin-1-yl)propan-1-one. MS (ESI): Mass calcd. for C₂₃H₂₅N₇O₄, 463.5; m/z found, 464.2 [M+H]⁺. ¹H NMR (400 MHz, DMSO-d₆) δ 8.53 (d, J=5.0 Hz, 1H), 8.46-8.41 (m, 1H), 8.25-8.10 (m, 2H), 7.67 (d, J=4.9 Hz, 1H), 7.54-7.28 (m, 1H), 7.15 (s, 1H), 6.78 (s, 1H), 4.52-4.36 (m, 2H), 4.29-4.15 (m, 1H), 3.96-3.79 (m, 2H), 3.52-3.42 (m, 2H), 2.85 (s, 3H), 2.66-2.58 (m, 1H), 2.31-2.21 (m, 3H), 1.33 (d, J=6.9 Hz, 3H).

### Example 169: (R)-3-(3-(6-(2-(((R)-1-(3,3-Difluoroazetidin-1-yl)-1-oxopropan-2-yl)amino)pyrimidin-4-yl)pyridin-2-yl)isoxazol-5-yl)-3-hydroxy-1-methylpyrrolidin-2-one

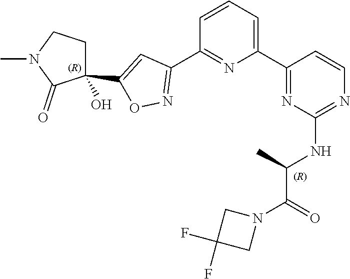

The title compound (13 mg, 22%) was prepared using conditions analogous to those described in Example 167 using (R)-2-amino-1-(3,3-difluoroazetidin-1-yl)propan-1-one (Intermediate 58) in place of (R)-2-amino-1-(azetidin-1-yl)propan-1-one. MS (ESI): Mass calcd. for C₂₃H₂₃F₂N₇O₄, 499.5; m/z found, 500.2 [M+H]⁺. ¹H NMR (400 MHz, DMSO-d₆) δ 8.59-8.53 (m, 1H), 8.46-8.40 (m, 1H), 8.18-8.12 (m, 2H), 7.73-7.67 (m, 1H), 7.33-7.29 (m, 1H), 7.13 (s, 1H), 6.54 (s, 1H), 4.90-4.26 (m, 5H), 4.90-4.26 (m, 1H), 4.90-4.26 (m, 1H), 3.59-3.42 (m, 2H), 2.89 (s, 3H), 2.69-2.61 (m, 1H), 2.42-2.30 (m, 1H), 1.47-1.41 (m, 3H).

### Example 170: (R)-3-(3-(6-(2-(((S)-1-(3,3-Difluoroazetidin-1-yl)-1-oxopropan-2-yl)amino)pyrimidin-4-yl)pyridin-2-yl)isoxazol-5-yl)-3-hydroxy-1-methylpyrrolidin-2-one

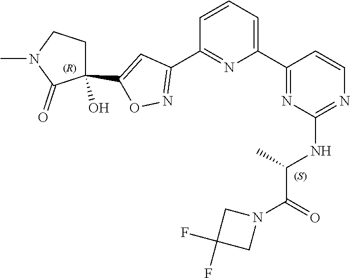

The title compound (3 mg, 3%) was prepared using conditions analogous to those described in Example 167 using (S)-2-amino-1-(3,3-difluoroazetidin-1-yl)propan-1-one trifluoroacetate (Intermediate 59) in place of (R)-2-amino-1-(azetidin-1-yl)propan-1-one. MS (ESI): Mass calcd. for C₂₃H₂₃F₂N₇O₄, 499.5; m/z found, 500.2 [M+H]⁺. ¹H NMR (400 MHz, DMSO-d₆) δ 8.53 (d, J=4.9 Hz, 1H), 8.39 (br s, 1H), 8.18-8.06 (m, 2H), 7.73-7.58 (m, 2H), 7.15 (s, 1H), 6.78 (s, 1H), 4.90 (s, 1H), 4.79-4.63 (m, 1H), 4.54-4.27 (m, 3H), 3.53-3.43 (m, 2H), 2.85 (s, 3H), 2.65-2.59 (m, 1H), 2.32-2.25 (m, 1H), 1.38 (d, J=6.9 Hz, 3H).

### Example 171: (R)-3-Hydroxy-1-methyl-3-(3-(6-(2-(((R)-1-oxo-1-(3-(trifluoromethyl)azetidin-1-yl)propan-2-yl)amino)pyrimidin-4-yl)pyridin-2-yl)isoxazol-5-yl)pyrrolidin-2-one

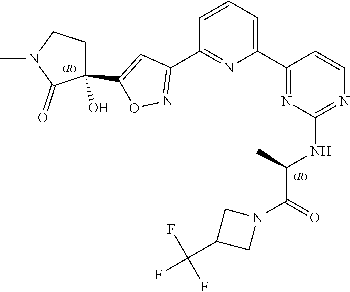

The title compound (19 mg, 30%) was prepared using conditions analogous to those described in Example 167 using (R)-2-amino-1-(3-(trifluoromethyl)azetidin-1-yl)propan-1-one in place of (R)-2-amino-1-(azetidin-1-yl)propan-1-one. MS (ESI): Mass calcd. for C₂₄H₂₄F₃N₇O₄, 531.5; m/z found, 532.2 [M+H]⁺. ¹H NMR (400 MHz, DMSO-d₆) δ 8.54-8.48 (m, 1H), 8.44-8.37 (m, 1H), 8.16-8.07 (m, 2H), 7.68-7.62 (m, 1H), 7.22-7.15 (m, 1H), 7.09 (s, 1H), 6.50 (s, 1H), 4.58-3.76 (m, 5H), 3.67-3.54 (m, 1H), 3.52-3.38 (m, 2H), 2.85 (s, 3H), 2.67-2.56 (m, 1H), 2.35-2.27 (m, 1H), 1.39-1.32 (m, 3H).

### Example 172: (R)-3-Hydroxy-1-methyl-3-(3-(6-(2-(((S)-1-oxo-1-(3-(trifluoromethyl)azetidin-1-yl)propan-2-yl)amino)pyrimidin-4-yl)pyridin-2-yl)isoxazol-5-yl)pyrrolidin-2-one

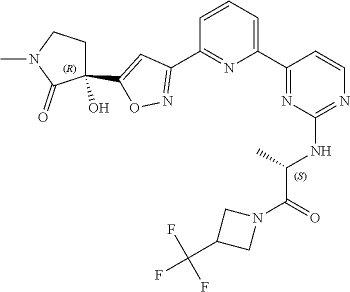

The title compound (20 mg, 20%) was prepared using conditions analogous to those described in Example 167 using (S)-2-amino-1-(3-(trifluoromethyl)azetidin-1-yl)propan-1-one trifluoroacetate (Intermediate 60) in place of (R)-2-amino-1-(azetidin-1-yl)propan-1-one. MS (ESI): Mass calcd. for C₂₄H₂₄F₃N₇O₄, 531.5; m/z found, 532.2 [M+H]⁺. ¹H NMR (400 MHz, DMSO-d₆) δ 8.57-8.47 (m, 1H), 8.45-8.38 (m, 1H), 8.26-8.03 (m, 2H), 7.73-7.65 (m, 1H), 7.63-7.49 (m, 1H), 7.15 (d, J=2.9 Hz, 1H), 6.78 (s, 1H), 4.71-4.01 (m, 4H), 3.92-3.79 (m, 1H), 3.74-3.58 (m, 1H), 3.53-3.42 (m, 2H), 2.85 (s, 3H), 2.66-2.58 (m, 1H), 2.32-2.25 (m, 1H), 1.35 (d, J=5.2 Hz, 3H).

### Example 173: (R)-3-(3-(6-(2-(((R)-1-(3,3-Difluoropyrrolidin-1-yl)-1-oxopropan-2-yl)amino)pyrimidin-4-yl)pyridin-2-yl)isoxazol-5-yl)-3-hydroxy-1-methylpyrrolidin-2-one

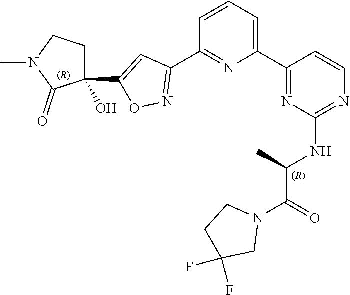

The title compound (33 mg, 52%) was prepared using conditions analogous to those described in Example 167 using (R)-2-amino-1-(3,3-difluoropyrrolidin-1-yl)propan-1-one in place of (R)-2-amino-1-(azetidin-1-yl)propan-1-one. MS (ESI): Mass calcd. for C₂₄H₂₅F₂N₇O₄, 513.5; m/z found, 514.3 [M+H]⁺. ¹H NMR (400 MHz, DMSO-d₆) δ 8.54-8.48 (m, 1H), 8.39-8.33 (m, 1H), 8.09 (br s, 1H), 7.67-7.60 (m, 1H), 7.17 (br s, 1H), 7.09 (s, 1H), 6.50 (s, 1H), 4.84-4.61 (m, 1H), 4.35-3.54 (m, 4H), 3.52-3.38 (m, 2H), 2.85 (s, 3H), 2.67-2.57 (m, 1H), 2.51 (br s, 2H), 2.35-2.27 (m, 1H), 1.42-1.35 (m, 3H).

### Example 174: (R)-3-(3-(6-(2-(((S)-1-(3,3-Difluoropyrrolidin-1-yl)-1-oxopropan-2-yl)amino)pyrimidin-4-yl)pyridin-2-yl)isoxazol-5-yl)-3-hydroxy-1-methylpyrrolidin-2-one

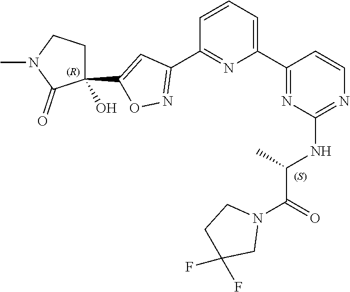

The title compound (5.2 mg, 5%) was prepared using conditions analogous to those described in Example 167 using (S)-2-amino-1-(3,3-difluoropyrrolidin-1-yl)propan-1-one in place of (R)-2-amino-1-(azetidin-1-yl)propan-1-one. MS (ESI): Mass calcd. for C₂₄H₂₅F₂N₇O₄, 513.5; m/z found, 514.2 [M+H]⁺. ¹H NMR (400 MHz, DMSO-d₆) δ 8.52 (d, J=4.8 Hz, 1H), 8.46-8.23 (m, 1H), 8.19-7.99 (m, 2H), 7.72-7.49 (m, 2H), 7.15 (s, 1H), 6.78 (br s, 1H), 4.76-4.52 (m, 1H), 4.40-3.62 (m, 3H), 3.60-3.41 (m, 3H), 2.85 (s, 3H), 2.66-2.54 (m, 2H), 2.45-2.34 (m, 1H), 2.32-2.23 (m, 1H), 1.40-1.32 (m, 3H).

### Example 175: (R)-2-((4-(6-(5-((R)-3-Hydroxy-1-methyl-2-oxopyrrolidin-3-yl)isoxazol-3-yl)pyridin-2-yl)pyrimidin-2-yl)amino)-N,N-dimethylpropanamide

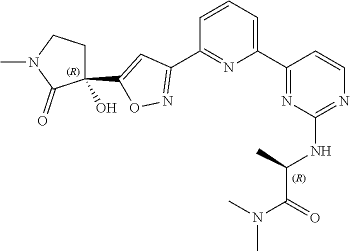

The title compound was prepared using conditions analogous to those described in Example 167 using (R)-2-amino-N,N-dimethylpropanamide in place of (R)-2-amino-1-(azetidin-1-yl)propan-1-one. The reaction mixture was purified directly by reverse-phase HPLC (Xtimate C18 column, 5 μm, 30×150 mm; 22-52% ACN/H₂O (with 0.05% NH₃ in water and 10 mM NH₄HCO₃)) to provide a white solid. The solid obtained was further purified sequentially by SFC (DAICEL CHIRALCEL OD-H, 5 μm, 30×250 mm, isocratic elution: 45% EtOH (0.1% of aqueous NH₃), 55% CO₂) and reverse-phase HPLC (Xtimate C18 column, 5 μm, 30×150 mm; 21-51% ACN/H₂O (with 0.05% NH₃ in water and 10 mM NH₄HCO₃)) to afford the title compound as a white solid (4.8 mg, 4.4%). MS (ESI): Mass calcd. for C₂₂H₂₅N₇O₄, 451.5; m/z found, 452.2 [M+H]⁺. ¹H NMR (400 MHz, DMSO-d₆) δ 8.52 (d, J=5.2 Hz, 1H), 8.39 (br s, 1H), 8.21-8.10 (m, 2H), 7.66 (d, J=5.2 Hz, 1H), 7.48-7.20 (m, 1H), 7.15 (s, 1H), 6.78 (s, 1H), 5.00-4.87 (m, 1H), 3.53-3.41 (m, 2H), 3.25-3.07 (m, 3H), 2.91-2.80 (m, 6H), 2.66-2.57 (m, 1H), 2.34-2.24 (m, 1H), 1.34 (d, J=6.8 Hz, 3H).

### Example 176: (S)-2-((4-(6-(5-((R)-3-Hydroxy-1-methyl-2-oxopyrrolidin-3-yl)isoxazol-3-yl)pyridin-2-yl)pyrimidin-2-yl)amino)-N,N-dimethylpropanamide

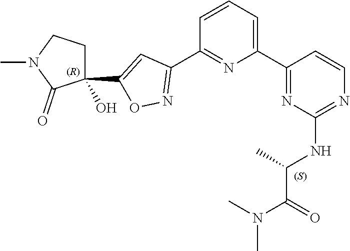

The title compound was prepared using conditions analogous to those described in Example 167 using (S)-2-amino-N,N-dimethylpropanamide in place of (R)-2-amino-1-(azetidin-1-yl)propan-1-one. The reaction mixture was concentrated and purified directly by reverse-phase HPLC (Xtimate C18 column, 5 μm, 30×150 mm; 20-50% ACN/H₂O (with 0.05% NH₃ in water and 10 mM NH₄HCO₃)) to provide the title compound as a colorless solid (51 mg, 59%). MS (ESI): Mass calcd. for C₂₂H₂₅N₇O₄, 451.5; m/z found, 452.2 [M+H]⁺. ¹H NMR (400 MHz, DMSO-d₆) δ 8.52 (d, J=5.0 Hz, 1H), 8.39 (br s, 1H), 8.24-8.10 (m, 2H), 7.70-7.61 (m, 1H), 7.50-7.22 (m, 1H), 7.15 (s, 1H), 6.78 (s, 1H), 5.12-4.81 (m, 1H), 3.50-3.41 (m, 2H), 3.25-3.07 (m, 3H), 2.89-2.83 (m, 6H), 2.65-2.59 (m, 1H), 2.32-2.25 (m, 1H), 1.38-1.30 (m, 3H).

### Example 177: (R)-3-(3-(6-(2-(((R)-1-(3-Fluoro-3-methylazetidin-1-yl)-1-oxopropan-2-yl)amino)pyrimidin-4-yl)pyridin-2-yl)isoxazol-5-yl)-3-hydroxy-1-methylpyrrolidin-2-one

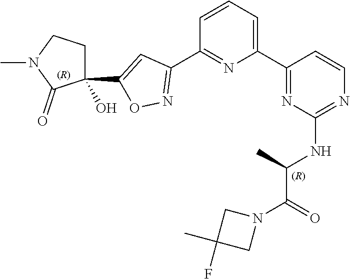

The title compound was prepared using conditions analogous to those described in Example 167 using (R)-2-amino-1-(3-fluoro-3-methylazetidin-1-yl)propan-1-one (Intermediate 61) in place of (R)-2-amino-1-(azetidin-1-yl)propan-1-one. The reaction mixture was purified directly by reverse-phase HPLC (Xtimate C18 column, 5 μm, 30×150 mm; 25-55% ACN/H₂O (with 0.05% NH₃ in water and 10 mM NH₄HCO₃)) to provide the title compound as a white solid (10 mg, 8%). MS (ESI): Mass calcd. for C₂₄H₂₆FN₇O₄, 495.5; m/z found, 496.2 [M+H]⁺. ¹H NMR (400 MHz, DMSO-d₆) δ 8.57-8.50 (m, 1H), 8.47-8.39 (m, 1H), 8.23-8.03 (m, 2H), 7.71-7.64 (m, 1H), 7.63-7.45 (m, 1H), 7.15 (s, 1H), 6.78 (s, 1H), 4.61-4.40 (m, 2H), 4.39-4.20 (m, 1H), 4.05-3.87 (m, 2H), 3.52-3.43 (m, 2H), 2.85 (s, 3H), 2.66-2.58 (m, 1H), 2.32-2.25 (m, 1H), 1.66-1.48 (m, 3H), 1.41-1.30 (m, 3H).

### Example 178: (R)-3-(3-(6-(2-(((S)-1-(3-Fluoro-3-methylazetidin-1-yl)-1-oxopropan-2-yl)amino)pyrimidin-4-yl)pyridin-2-yl)isoxazol-5-yl)-3-hydroxy-1-methylpyrrolidin-2-one

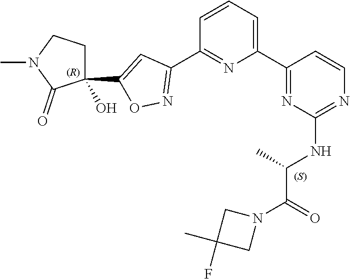

The title compound was prepared using conditions analogous to those described in Example 167 using (S)-2-amino-1-(3-fluoro-3-methylazetidin-1-yl)propan-1-one (Intermediate 62) in place of (R)-2-amino-1-(azetidin-1-yl)propan-1-one. The reaction mixture was concentrated and purified directly by reverse-phase HPLC (Xtimate C18 column, 5 μm, 30×150 mm; 23-53% ACN/H₂O (with 0.05% NH₃ in water and 10 mM NH₄HCO₃)) to provide a yellow solid. The solid obtained was further purified by SFC (DAICEL CHIRALPAK AS, 10 μm, 30×250 mm, isocratic elution: 40% EtOH (0.1% of aqueous NH₃), 60% CO₂) to provide the title compound (4.5 mg, 5%). MS (ESI): Mass calcd. for C₂₄H₂₆FN₇O₄, 495.5; m/z found, 496.2 [M+H]⁺. ¹H NMR (400 MHz, DMSO-d₆) δ 8.53 (d, J=4.9 Hz, 1H), 8.48-8.37 (m, 1H), 8.20-8.10 (m, 2H), 7.68 (d, J=4.9 Hz, 1H), 7.64-7.48 (m, 1H), 7.15 (s, 1H), 6.78 (s, 1H), 4.64-4.39 (m, 2H), 4.38-4.23 (m, 1H), 4.07-3.85 (m, 2H), 3.52-3.42 (m, 2H), 2.85 (s, 3H), 2.64-2.59 (m, 1H), 2.31-2.24 (m, 1H), 1.66-1.52 (m, 3H), 1.43-1.31 (m, 3H).

### Example 179: (R)-3-Hydroxy-1-methyl-3-(3-(6-(2-(((R)-1-oxo-1-(piperidin-1-yl)propan-2-yl)amino)pyrimidin-4-yl)pyridin-2-yl)isoxazol-5-yl)pyrrolidin-2-one

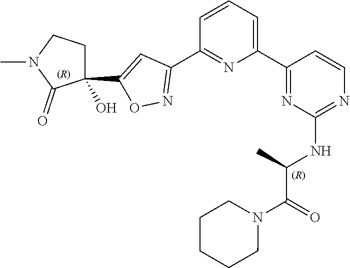

The title compound was prepared using conditions analogous to those described in Example 167 using (R)-2-amino-1-(piperidin-1-yl)propan-1-one in place of (R)-2-amino-1-(azetidin-1-yl)propan-1-one. The reaction mixture was purified directly by reverse-phase HPLC (Xtimate C18 column, 5 μm, 30×150 mm; 28-58% ACN/H₂O (with 0.05% NH₃ in water and 10 mM NH₄HCO₃)) to provide a white solid. The solid obtained was further purified by SFC (DAICEL CHIRALCEL OD-H, 5 μm, 30×250 mm, isocratic elution: 50% EtOH (0.1% of aqueous NH₃), 50% CO₂) to provide the title compound as a white solid (26 mg, 22%). MS (ESI): Mass calcd. for C₂₅H₂₉N₇O₄, 491.5; m/z found, 492.2 [M+H]⁺. ¹H NMR (400 MHz, DMSO-d₆) δ 8.54-8.49 (m, 1H), 8.48-8.42 (m, 1H), 8.20-8.11 (m, 2H), 7.70-7.62 (m, 1H), 7.44-7.20 (m, 1H), 7.15 (s, 1H), 6.77 (s, 1H), 5.03-4.88 (m, 1H), 3.69-3.38 (m, 6H), 2.85 (s, 3H), 2.66-2.58 (m, 1H), 2.35-2.25 (m, 1H), 1.65-1.49 (m, 4H), 1.47-1.39 (m, 2H), 1.34 (br d, J=6.8 Hz, 3H).

### Example 180: (R)-3-Hydroxy-1-methyl-3-(3-(6-(2-(((S)-1-oxo-1-(piperidin-1-yl)propan-2-yl)amino)pyrimidin-4-yl)pyridin-2-yl)isoxazol-5-yl)pyrrolidin-2-one

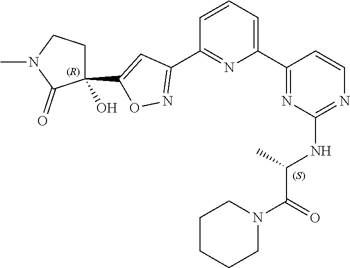

The title compound was prepared using conditions analogous to those described in Example 167 using (S)-2-amino-1-(piperidin-1-yl)propan-1-one in place of (R)-2-amino-1-(azetidin-1-yl)propan-1-one. The reaction mixture was concentrated and purified directly by reverse-phase HPLC (Boston Prime C18 column, 5 μm, 30×150 mm; 35-65% ACN/H₂O (with 0.05% NH₃ in water and 10 mM NH₄HCO₃)) to provide a white solid. The solid obtained was further purified by SFC (DAICEL CHIRALPAK AS, 10 m, 30×250 mm, isocratic elution: 50% EtOH (0.1% of aqueous NH₃), 50% CO₂) to provide the title compound (6.4 mg, 6.7%). MS (ESI): Mass calcd. for C₂₅H₂₉N₇O₄, 491.5; m/z found, 492.2 [M+H]⁺. 1H NMR (400 MHz, DMSO-d₆) δ 8.51 (d, J=5.0 Hz, 1H), 8.47-8.40 (m, 1H), 8.20-8.10 (m, 2H), 7.66 (d, J=5.0 Hz, 1H), 7.44-7.24 (m, 1H), 7.15 (s, 1H), 6.78 (s, 1H), 5.15-4.83 (m, 1H), 3.66-3.40 (m, 6H), 2.85 (s, 3H), 2.65-2.59 (m, 1H), 2.32-2.25 (m, 1H), 1.66-1.48 (m, 4H), 1.47-1.38 (m, 2H), 1.37-1.28 (m, 3H).

### Example 181: (S)-2-((4-(6-(5-((R)-3-Hydroxy-1-methyl-2-oxopyrrolidin-3-yl)isoxazol-3-yl)pyridin-2-yl)pyrimidin-2-yl)amino)-N-methyl-N-phenylpropanamide

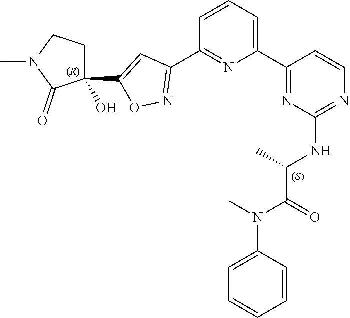

The title compound was prepared using conditions analogous to those described in Example 167 using (S)-2-amino-N-methyl-N-phenylpropanamide trifluoroacetate (Intermediate 63) in place of (R)-2-amino-1-(azetidin-1-yl)propan-1-one. The reaction mixture was concentrated and purified directly by reverse-phase HPLC (Boston Prime C18 column, 5 μm, 30×150 mm; 40-70% ACN/H₂O (with 0.05% NH₃ in water and 10 mM NH₄HCO₃)) to provide the title compound as a yellow solid (19 mg, 19%). MS (ESI): Mass calcd. for C₂₇H₂₇N₇O₄, 513.6; m/z found, 514.3 [M+H]⁺. ¹H NMR (400 MHz, DMSO-d₆) δ 8.59-8.02 (m, 4H), 7.77-7.21 (m, 7H), 7.15 (s, 1H), 6.78 (s, 1H), 4.97-4.34 (m, 1H), 3.52-3.42 (m, 2H), 3.27-3.10 (m, 3H), 2.85 (s, 3H), 2.66-2.59 (m, 1H), 2.32-2.25 (m, 1H), 1.29-1.14 (m, 3H).

### Example 182: (S)-2-((4-(6-(5-((R)-3-Hydroxy-1-methyl-2-oxopyrrolidin-3-yl)isoxazol-3-yl)pyridin-2-yl)pyrimidin-2-yl)amino)propanamide

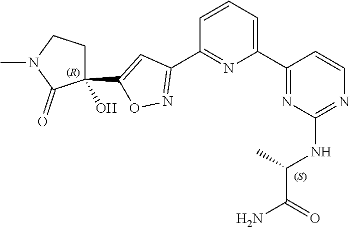

The title compound was prepared using conditions analogous to those described in Example 167 using (S)-2-aminopropanamide hydrochloride in place of (R)-2-amino-1-(azetidin-1-yl)propan-1-one. The reaction mixture was concentrated and purified directly by reverse-phase HPLC (Boston Prime C18 column, 5 μm, 30×150 mm; 15-45% ACN/H₂O (with 0.05% NH₃ in water and 10 mM NH₄HCO₃)) to provide the title compound (42 mg, 40%). MS (ESI): Mass calcd. for C₂₀H₂₁N₇O₄, 423.4; m/z found, 424.2 [M+H]⁺. ¹H NMR (400 MHz, DMSO-d₆) δ 8.54-8.51 (m, 1H), 8.50-8.44 (m, 1H), 8.18-8.10 (m, 2H), 7.68 (d, J=4.8 Hz, 1H), 7.47-7.21 (m, 2H), 7.17-7.13 (m, 1H), 6.96 (s, 1H), 6.80-6.75 (m, 1H), 4.44-4.35 (m, 1H), 3.53-3.42 (m, 2H), 2.85 (s, 3H), 2.65-2.58 (m, 1H), 2.32-2.24 (m, 1H), 1.38 (d, J=7.3 Hz, 3H).

### Example 183: (S)-2-((4-(6-(5-((R)-3-Hydroxy-1-methyl-2-oxopyrrolidin-3-yl)isoxazol-3-yl)pyridin-2-yl)pyrimidin-2-yl)amino)-N-methylpropanamide

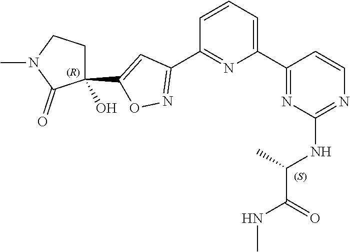

The title compound (55 mg, 50%) was prepared using conditions analogous to those described in Example 167 using (S)-2-amino-N-methylpropanamide hydrochloride in place of (R)-2-amino-1-(azetidin-1-yl)propan-1-one. MS (ESI): Mass calcd. for C₂₁H₂₃N₇O₄, 437.5; m/z found, 438.2 [M+H]⁺. ¹H NMR (400 MHz, DMSO-d₆) δ 8.52 (d, J=5.2 Hz, 1H), 8.48-8.42 (m, 1H), 8.21-8.07 (m, 2H), 8.01-7.76 (m, 1H), 7.68 (d, J=5.2 Hz, 1H), 7.51-7.25 (m, 1H), 7.15 (s, 1H), 6.77 (s, 1H), 4.40 (t, J=6.8 Hz, 1H), 3.57-3.37 (m, 2H), 2.93-2.80 (m, 3H), 2.65-2.61 (m, 1H), 2.59 (d, J=4.8 Hz, 3H), 2.31-2.24 (m, 1H), 1.36 (d, J=7.2 Hz, 3H).

### Example 184: (R)-3-Hydroxy-1-methyl-3-(3-(6-(2-(((S)-1-oxo-1-(pyrrolidin-1-yl)propan-2-yl)amino)pyrimidin-4-yl)pyridin-2-yl)isoxazol-5-yl)pyrrolidin-2-one

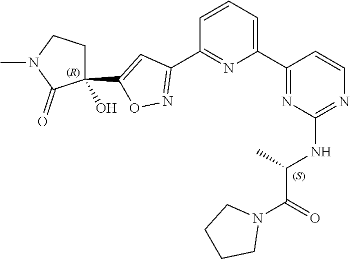

The title compound was prepared using conditions analogous to those described in Example 167 using (S)-2-amino-1-(pyrrolidin-1-yl)propan-1-one in place of (R)-2-amino-1-(azetidin-1-yl)propan-1-one. The reaction mixture was purified directly by reverse-phase HPLC (Xtimate C18 column, 5 μm, 30×150 mm; 20-50% ACN/H₂O (with 0.05% NH₃ in water and 10 mM NH₄HCO₃)) to provide the title compound as a white solid (56 mg, 47%). MS (ESI): Mass calcd. for C₂₄H₂₇N₇O₄, 477.5; m/z found, 478.2 [M+H]⁺. ¹H NMR (400 MHz, DMSO) δ 8.52 (d, J=5.0 Hz, 1H), 8.48-8.24 (m, 1H), 8.20-8.11 (m, 2H), 7.68-7.60 (m, 1H), 7.52-7.25 (m, 1H), 7.15 (s, 1H), 6.78 (s, 1H), 4.75-4.60 (m, 1H), 3.84-3.70 (m, 1H), 3.59-3.39 (m, 3H), 3.33-3.24 (m, 2H), 2.85 (s, 3H), 2.66-2.59 (m, 1H), 2.36-2.23 (m, 1H), 2.03-1.74 (m, 4H), 1.35 (d, J=6.9 Hz, 3H).

### Example 185: (R)-3-(3-(6-(2-((2-(3,3-Difluoroazetidin-1-yl)-2-oxoethyl)amino)pyrimidin-4-yl)pyridin-2-yl)isoxazol-5-yl)-3-hydroxy-1-methylpyrrolidin-2-one

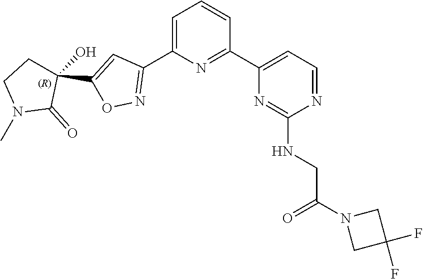

The title compound was prepared using conditions analogous to those described in Example 167 using 2-amino-1-(3,3-difluoroazetidin-1-yl)ethan-1-one hydrochloride (Intermediate 64) in place of (R)-2-amino-1-(azetidin-1-yl)propan-1-one. The reaction mixture was purified directly by reverse-phase HPLC (Xtimate C18 column, 5 μm, 25×150 mm; 33-63% ACN/H₂O (with 0.05% NH₃ in water and 10 mM NH₄HCO₃)) to provide the title compound as a white solid (23 mg, 19%). MS (ESI): Mass calcd. for C₂₂H₂₁F₂N₇O₄, 485.5; m/z found, 486.1 [M+H]⁺. ¹H NMR (400 MHz, CDCl₃) δ 8.57 (s, 1H), 8.34 (d, J=7.6 Hz, 1H), 8.08 (d, J=7.6 Hz, 1H), 7.95-7.83 (m, 1H), 7.74 (d, J=5.2 Hz, 1H), 7.13 (s, 1H), 4.57-4.28 (m, 4H), 4.21-4.06 (m, 2H), 3.75-3.63 (m, 1H), 3.58-3.43 (m, 1H), 3.03 (s, 3H), 2.82-2.69 (m, 1H), 2.62-2.41 (m, 1H).

### Example 186: (R)-3-Hydroxy-3-(3-(6-(2-(isoxazol-3-ylamino)pyrimidin-4-yl)pyridin-2-yl)isoxazol-5-yl)-1-methylpyrrolidin-2-one

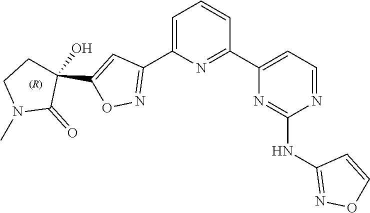

A mixture of (R)-3-hydroxy-1-methyl-3-(3-(6-(2-(methylsulfonyl)pyrimidin-4-yl)pyridin-2-yl)isoxazol-5-yl)pyrrolidin-2-one (200 mg, 0.481 mmol, Intermediate 13), isoxazole-3-amine (290 mg, 2.41 mmol) and TsOH (100 mg, 0.581 mmol) in 1,4-dioxane (5 mL) was sparged with N₂ for 5 min, then heated at 115° C. for 16 hrs. The mixture was cooled to room temperature, diluted with water (5 mL) and extracted with EtOAc (3×10 mL). The combined organic layers were washed with brine (8 mL), dried over anhydrous Na₂SO₄, filtered and concentrated. The residue was purified sequentially by silica gel chromatography (0-10% MeOH/DCM) and then reverse-phase HPLC (Boston Prime C18 column, 5 μm, 30×150 mm; 25-55% ACN/H₂O (with 0.05% NH₃ in water and 10 mM NH₄HCO₃)) to provide the title compound as a white solid (8 mg, 5%). MS (ESI): Mass calcd. for C₂₀H₁₇N₇O₄, 419.4; m/z found, 420.1 [M+H]⁺. ¹H NMR (400 MHz, DMSO-d₆) δ 8.74-8.69 (m, 2H), 8.48-8.44 (m, 1H), 8.22-8.11 (m, 2H), 7.96-7.92 (m, 1H), 7.15-7.11 (m, 2H), 6.50 (br s, 1H), 3.52-3.42 (m, 2H), 2.85 (s, 3H), 2.64-2.59 (m, 1H), 2.36-2.32 (m, 1H).

### Example 187: (S)-3-Hydroxy-1-methyl-3-(3-(6-(2-((5-methyl-1-(tetrahydro-2H-pyran-4-yl)-1H-pyrazol-4-yl)amino)pyrimidin-4-yl)pyridin-2-yl)isoxazol-5-yl)pyrrolidin-2-one

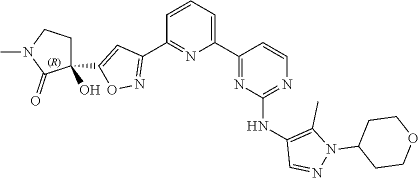

The title compound (52 mg, 41%) was prepared using conditions analogous to those described in Example 186 using 5-methyl-1-(tetrahydro-2H-pyran-4-yl)-1H-pyrazol-4-amine in place of isoxazole-3-amine. MS (ESI): Mass calcd. for C₂₆H₂₈N₈O₄, 516.6; m/z found, 517.2 [M+H]⁺. ¹H NMR (400 MHz, DMSO-d₆) δ 8.92 (br s, 1H), 8.63-8.49 (m, 1H), 8.44-8.31 (m, 1H), 8.22-8.09 (m, 2H), 7.74 (d, J=5.0 Hz, 2H), 7.16 (s, 1H), 6.78 (s, 1H), 4.45-4.32 (m, 1H), 4.04-3.93 (m, 2H), 3.54-3.43 (m, 4H), 2.85 (s, 3H), 2.66-2.58 (m, 1H), 2.32-2.22 (m, 4H), 2.11-2.00 (m, 2H), 1.86-1.76 (m, 2H).

### Example 188: (*R)-3-(5-(6-(2-((1-(2,2-Difluoroethyl)-3-methoxy-1H-pyrazol-4-yl)amino)pyrimidin-4-yl)pyridin-2-yl)thiazol-2-yl)-3-hydroxy-1-methylpyrrolidin-2-one

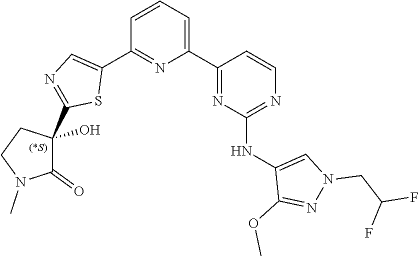

The title compound was prepared using conditions analogous to those described in Example 166 using 3-(5-(6-chloropyridin-2-yl)thiazol-2-yl)-3-hydroxy-1-methylpyrrolidin-2-one (Intermediate 27) in place of 3-(5-(6-chloropyridin-2-yl)-1-methyl-1H-pyrazol-3-yl)-3-hydroxy-1-methylpyrrolidin-2-one and N-(1-(2,2-difluoroethyl)-3-methoxy-1H-pyrazol-4-yl)-4-(tributylstannyl)pyrimidin-2-amine (Intermediate 66) in place of N-(1-methyl-1H-pyrazol-3-yl)-4-(tributylstannyl)pyrimidin-2-amine. The residue was purified by reverse-phase HPLC (Boston Prime C18 column, 5 μm, 30×150 mm; 35-65% ACN/H₂O (with 0.05% NH₃ in water and 10 mM NH₄HCO₃)) to provide a mixture of the (R) and (S) enantiomers of 3-(5-(6-(2-((1-(2,2-difluoroethyl)-3-methoxy-1H-pyrazol-4-yl)amino)pyrimidin-4-yl)pyridin-2-yl)thiazol-2-yl)-3-hydroxy-1-methylpyrrolidin-2-one. The (R) and (S) enantiomers were separated by chiral SFC (DAICEL CHIRALPAK AS, 10 μm, 30×250 mm, isocratic elution: 50% EtOH (0.1% of aqueous NH₃), 50% CO₂) to provide Example 188 as the second-eluting isomer (32 mg, 36%) which was designated as the (*R) enantiomer. MS (ESI): Mass calcd. for C₂₃H₂₂F₂N₈O₃S, 528.5; m/z found, 529.2 [M+H]⁺. ¹H NMR (500 MHz, DMSO-d₆) δ 8.69 (br s, 1H), 8.61-8.55 (m, 1H), 8.47 (s, 1H), 8.27-8.18 (m, 1H), 8.12-8.03 (m, 2H), 7.93 (br s, 1H), 7.68-7.64 (m, 1H), 6.96 (s, 1H), 6.46-6.18 (m, 1H), 4.56-4.40 (m, 2H), 3.84 (s, 3H), 3.53-3.45 (m, 2H), 2.82 (s, 3H), 2.76-2.68 (m, 1H), 2.31-2.23 (m, 1H).

### Example 189: (*R)-3-(5-(6-(2-((1-(2,2-Difluoroethyl)-3-methoxy-1H-pyrazol-4-yl)amino)pyrimidin-4-yl)pyridin-2-yl)-1H-pyrazol-3-yl)-3-hydroxy-1-methylpyrrolidin-2-one

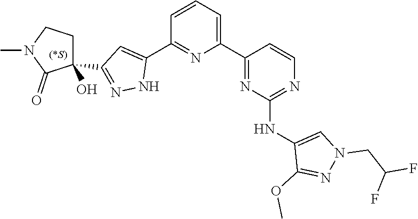

Step A. 3-(3-(6-Chloropyridin-2-yl)-1-((2-(trimethylsilyl)ethoxy)methyl)-1H-pyrazol-5-yl)-3-hydroxy-1-methylpyrrolidin-2-one. To a mixture of 3-(3-bromo-1-((2-(trimethylsilyl)ethoxy)methyl)-1H-pyrazol-5-yl)-3-hydroxy-1-methylpyrrolidin-2-one (1.0 g, 2.6 mmol, Intermediate 29), 2-chloro-6-(4,4,5,5-tetramethyl-1,3,2-dioxaborolan-2-yl)pyridine (675 mg, 2.82 mmol) and K₂CO₃ (1.06 g, 7.69 mmol) in 1,4-dioxane:water (15 mL, 4:1) was added Pd(dppf)Cl₂ (187 mg, 0.256 mmol) and the mixture was purged with N₂ for 5 min, then heated at 110° C. for 16 hrs. The mixture was diluted with water (10 mL) and extracted with EtOAc (3×10 mL). The combined organic layers were washed with brine (10 mL), dried over anhydrous Na₂SO₄, filtered and concentrated. The residue was purified by reverse-phase HPLC (Xtimate C18 column, 10 μm, 40×150 mm; 50-80% ACN/H₂O (with 0.05% NH₃ in water and 10 mM NH₄HCO₃)) to afford the title compound as a white solid (300 mg, 25%). MS (ESI⁺): m/z=423.2.

Step B. 3-(3-(6-(2-((1-(2,2-Difluoroethyl)-3-methoxy-1H-pyrazol-4-yl)amino)pyrimidin-4-yl)pyridin-2-yl)-1-((2-(trimethylsilyl)ethoxy)methyl)-1H-pyrazol-5-yl)-3-hydroxy-1-methylpyrrolidin-2-one. To a mixture of 3-(3-(6-chloropyridin-2-yl)-1-((2-(trimethylsilyl)ethoxy)methyl)-1H-pyrazol-5-yl)-3-hydroxy-1-methylpyrrolidin-2-one (250 mg, 0.591 mmol) and N-(1-(2,2-difluoroethyl)-3-methoxy-1H-pyrazol-4-yl)-4-(tributylstannyl)pyrimidin-2-amine (322 mg, 0.591 mmol, Intermediate 66) in 1,4-dioxane (5 mL) was added Pd(dppf)Cl₂ (43 mg, 0.059 mmol), and the resulting mixture was sparged with N₂ for 5 min then subjected to microwave irradiation at 110° C. for 1 h. The reaction mixture was cooled to room temperature, diluted with water (10 mL) and extracted with EtOAc (3×10 mL). The combined organic layers were washed with brine (10 mL), dried over anhydrous Na₂SO₄, filtered and concentrated. The residue was purified by silica gel chromatography (0-100% EtOAc/petroleum ether) to provide the title compound as a brown solid (280 mg, 70%). MS (ESI⁺): m/z=642.3.

Step C. (*S)-3-(5-(6-(2-((1-(2,2-Difluoroethyl)-3-methoxy-1H-pyrazol-4-yl)amino)pyrimidin-4-yl)pyridin-2-yl)-1H-pyrazol-3-yl)-3-hydroxy-1-methylpyrrolidin-2-one. A solution of 3-(3-(6-(2-((1-(2,2-difluoroethyl)-3-methoxy-1H-pyrazol-4-yl)amino)pyrimidin-4-yl)pyridin-2-yl)-1-((2-(trimethylsilyl)ethoxy)methyl)-1H-pyrazol-5-yl)-3-hydroxy-1-methylpyrrolidin-2-one (260 mg, 0.405 mmol) in TFA (1.0 mL, 13 mmol) was stirred at room temperature for 16 hrs. After that time, the mixture was purified directly by reverse-phase HPLC (Xtimate C18 column, 5 μm, 30×150 mm; 28-58% ACN/H₂O (with 0.05% NH₃ in water and 10 mM NH₄HCO₃)) to afford a mixture of enantiomers of 3-(5-(6-(2-((1-(2,2-difluoroethyl)-3-methoxy-1H-pyrazol-4-yl)amino)pyrimidin-4-yl)pyridin-2-yl)-1H-pyrazol-3-yl)-3-hydroxy-1-methylpyrrolidin-2-one. The (R) and (S) enantiomers were separated by chiral SFC (DAICEL CHIRALCEL OJ, 10 μm, 30×250 mm, isocratic elution: 75% EtOH (0.1% of aqueous NH₃), 25% CO₂) to provide Example 189 which was designated as the (*R) enantiomer as the second-eluting isomer (22 mg, 54%). MS (ESI): Mass calcd. for C₂₃H₂₃F₂N₉O₃, 511.5; m/z found, 512.2 [M+H]⁺. ¹H NMR (400 MHz, DMSO-d₆) δ 13.56-12.96 (m, 1H), 8.76-8.49 (m, 2H), 8.36-8.15 (m, 1H), 8.11-7.68 (m, 4H), 7.10-6.81 (m, 1H), 6.50-6.15 (m, 1H), 5.96 (br s, 1H), 4.57-4.40 (m, 2H), 3.85 (s, 2H), 3.88-3.82 (m, 1H), 3.46-3.37 (m, 2H), 2.87-2.70 (m, 4H), 2.32-2.09 (m, 1H).

### Example 190: (R)-3-Hydroxy-1-methyl-3-(1-(6-(2-((2-methylpyridin-3-yl)amino)pyrimidin-4-yl)pyridin-2-yl)-1H-1,2,3-triazol-4-yl)pyrrolidin-2-one

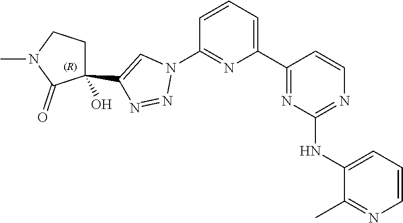

A flask containing N-(2-methylpyridin-3-yl)-4-(tributylstannyl)pyrimidin-2-amine (499 mg, 1.05 mmol, Intermediate 46), (R)-3-(1-(6-bromopyridin-2-yl)-1H-1,2,3-triazol-4-yl)-3-hydroxy-1-methylpyrrolidin-2-one (356 mg, 1.05 mmol, Intermediate 51) and TEA (335 μL, 2.40 mmol) in xylenes (5 mL) was evacuated and refilled with Ar three times, then Pd(PPh₃)₄ (135 mg, 0.117 mmol) was added and the resulting mixture was heated at 120° C. for 16 hrs. The reaction was gradually cooled to room temperature, saturated aqueous KF (15 mL) was added over 2 min, and the resulting mixture was stirred at room temperature for 2 hrs. The mixture was filtered and the filter cake was washed with CHCl₃/IPA (3/1, 60 mL). The combined filtrate was washed with water (3×15 mL), dried over anhydrous Na₂SO₄, filtered and concentrated. The residue was purified by reverse-phase HPLC (Xtimate C18 column, 5 μm, 30×150 mm; 23-53% ACN/H₂O (with 0.05% NH₃ in water and 10 mM NH₄HCO₃)) to provide, after lyophilization, the title compound as a yellow solid (284 mg, 60%). MS (ESI): Mass calcd. for C₂₂H₂₁N₉O₂, 443.5; m/z found, 444.2 [M+H]⁺. ¹H NMR (400 MHz, DMSO-d₆) δ 9.25 (s, 1H), 8.95 (s, 1H), 8.66 (d, J=4.8 Hz, 1H), 8.32-8.24 (m, 4H), 7.99 (d, J=7.6 Hz, 1H), 7.86 (d, J=4.8 Hz, 1H), 7.29 (dd, J=4.4, 8.0 Hz, 1H), 6.35 (s, 1H), 3.53-3.47 (m, 2H), 2.83 (s, 3H), 2.81-2.74 (m, 1H), 2.49 (s, 3H), 2.34-2.24 (m, 1H).

### Example 191: (R)-3-Hydroxy-1-methyl-3-(1-(6-(2-((2-methylpyridin-4-yl)amino)pyrimidin-4-yl)pyridin-2-yl)-1H-1,2,3-triazol-4-yl)pyrrolidin-2-one

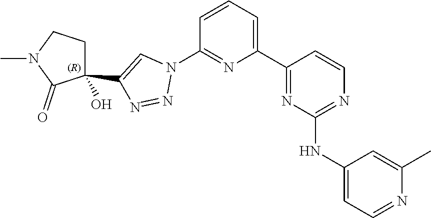

A mixture of (R)-3-(1-(6-bromopyridin-2-yl)-1H-1,2,3-triazol-4-yl)-3-hydroxy-1-methylpyrrolidin-2-one (120 mg, 0.355 mmol, Intermediate 51), N-(2-methylpyridin-4-yl)-4-(tributylstannyl)pyrimidin-2-amine (180 mg, 0.379 mmol, Intermediate 47), Pd(PPh₃)₄ (44 mg, 0.038 mmol) and TEA (100 μL, 0.717 mmol) in xylenes (5 mL) was sparged with N₂ for 5 min, then the mixture was heated at 120° C. for 16 hrs. The reaction mixture was cooled to room temperature, filtered and the filter cake was collected. The solids were purified by reverse-phase HPLC (Xtimate C18 column, 5 μm, 30×150 mm; 6-36% ACN/H₂O (with 0.05% formic acid)) to afford, after lyophilization, the title compound as a white solid (10 mg, 6%). MS (ESI): Mass calcd. for C₂₂H₂₁N₉O₂, 443.5; m/z found, 444.2 [M+H]⁺. ¹H NMR (400 MHz, DMSO-d₆) δ 10.27 (s, 1H), 8.97 (s, 1H), 8.82 (d, J=4.8 Hz, 1H), 8.49 (d, J=7.2 Hz, 1H), 8.41-8.27 (m, 3H), 8.16 (s, 0.33H), 8.01 (d, J=5.2 Hz, 1H), 7.76-7.67 (m, 2H), 3.53-3.49 (m, 2H), 2.84 (s, 3H), 2.81-2.75 (m, 1H), 2.45 (s, 3H), 2.35-2.25 (m, 1H).

### Example 192: (R)-3-(3-(6-(2-((6,6-Dimethyl-6,7-dihydro-4H-pyrazolo[5,1-c][1,4]oxazin-2-yl)amino)pyrimidin-4-yl)pyridin-2-yl)isoxazol-5-yl)-3-hydroxy-1-methylpyrrolidin-2-one

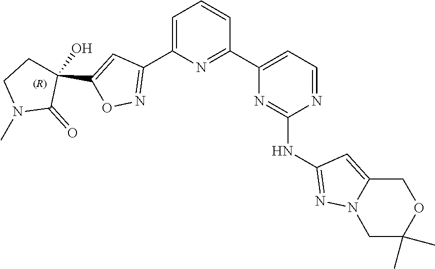

The title compound was prepared using conditions analogous to those described in Example 126 using 6,6-dimethyl-6,7-dihydro-4H-pyrazolo[5,1-c][1,4]oxazin-2-amine in place of 1-(2-(dimethylamino)ethyl)-1H-pyrazol-3-amine. The reaction mixture was concentrated and purified by SFC (DAICEL CHIRALPAK AS, 10 m, 30×250 mm, isocratic elution: 60% EtOH (0.1% of aqueous NH₃), 40% CO₂) to provide the title compound as a white solid (7 mg, 70%). MS (ESI): mass calcd. for C₂₅H₂₆N₈O₄, 502.5; m/z found, 503.2 [M+H]⁺. ¹H NMR (400 MHz, DMSO-d₆) δ 9.99 (s, 1H), 8.67-8.63 (m, 1H), 8.52-8.45 (m, 1H), 8.25-8.12 (m, 2H), 7.86-7.81 (m, 1H), 7.18 (s, 1H), 6.80 (s, 1H), 6.63 (s, 1H), 4.87-4.81 (m, 2H), 3.88-3.83 (m, 2H), 3.55-3.41 (m, 2H), 2.86 (s, 3H), 2.66-2.59 (m, 1H), 2.35-2.27 (m, 1H), 1.31 (s, 6H).

### Example 193: (R)-3-Hydroxy-1-methyl-3-(1-(6-(2-((1-methyl-1H-pyrazol-3-yl)amino)pyrimidin-4-yl-6-d)pyridin-2-yl)-1H-1,2,3-triazol-4-yl)pyrrolidin-2-one

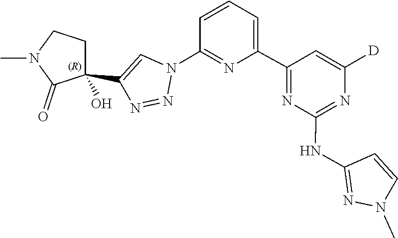

Step A. (R)-3-(1-(6-(2-Chloropyrimidin-4-yl-6-d)pyridin-2-yl)-1H-1,2,3-triazol-4-yl)-3-hydroxy-1-methylpyrrolidin-2-one. To a mixture of (R)-3-(1-(6-bromopyridin-2-yl)-1H-1,2,3-triazol-4-yl)-3-hydroxy-1-methylpyrrolidin-2-one (28.40 g, 83.98 mmol, Intermediate 51) and Pd(PPh₃)₄ (1.94 g, 1.68 mmol) was added THF (284 mL) and the resulting solution was heated to 60° C. Then, (2-chloropyrimidin-4-yl-6-d)zinc(II) chloride (280.8 mL, 0.75 M, Intermediate 50) was added and the mixture was heated at 60° C. for 1 h. After that time, the reaction mixture was poured into 0° C. aqueous NH₄Cl (200 mL) and the mixture was extracted with EtOAc (2×200 mL). The organic layers were combined, washed with brine (200 mL), dried over anhydrous Na₂SO₄, filtered and concentrated. The residue was purified by silica gel chromatography (0-10% MeOH/DCM) to provide the title compound as a brown solid (19.4 g, 55%). ¹H NMR (400 MHz, DMSO-d₆) δ 9.01 (s, 1H), 8.59 (s, 1H), 8.46 (dd, J=6.40, 1.20 Hz, 1H), 8.21-8.38 (m, 2H), 6.34 (s, 1H), 3.49 (br t, J=6.40 Hz, 2H), 2.70-2.87 (m, 4H), 2.27-2.32 (m, 1H). MS (ESI⁺): m/z=373.0.

Step B. (R)-3-Hydroxy-1-methyl-3-(1-(6-(2-((1-methyl-1H-pyrazol-3-yl)amino)pyrimidin-4-yl-6-d)pyridin-2-yl)-1H-1,2,3-triazol-4-yl)pyrrolidin-2-one. To a solution of (R)-3-(1-(6-(2-chloropyrimidin-4-yl-6-d)pyridin-2-yl)-1H-1,2,3-triazol-4-yl)-3-hydroxy-1-methylpyrrolidin-2-one (21.5 g, 57.7 mmol) in THF (420 mL) was added 1-methyl-1H-pyrazol-3-amine (11.2 g, 115 mmol), and the resulting solution was cooled to −40° C. Then, LiHMDS (231 mL, 231 mmol, 1 M in THF) was added and the mixture was stirred at −40° C. for 3 hrs. After that time, the reaction mixture was poured into 0° C. water (1 L) and the pH of the mixture was adjusted to pH˜10 by the addition of 2 M aqueous HCl. The mixture was concentrated to 1 L and the precipitate that formed was then filtered. The filtrate was extracted with DCM/MeOH (10/1, 3×500 mL). The organic layers and filter cake were combined and concentrated to afford a yellow solid. The solid was purified by silica gel chromatography (0-10% MeOH/DCM) to provide a yellow solid. To the solid was added DMF (190 mL) and the mixture was heated to 50° C. to provide a solution. Then, MeOH (48 mL) and THF (48 mL) were added sequentially and the mixture was allowed to stand at room temperature for 16 hrs. The suspension was poured into water (600 mL) and the resulting mixture was stirred at room temperature for 15 hrs. The suspension was then filtered and the filter cake washed with 0° C. MeOH (3×100 mL). The filtrate was concentrated to afford a yellow solid which was triturated with MeOH (100 mL) at 25° C. for 16 hrs. The yellow solid was dissolved in DCM (280 mL), then MeOH (100 mL) was added and the solution was concentrated down to 150 mL. Additional MeOH (300 mL) was added and the mixture was stirred at room temperature for 15 min. The mixture was filtered and the filtrate was concentrated to afford a yellow solid which was dissolved in MeOH (100 mL) and heated at 60° C. for 1 h. The mixture was concentrated and the residue dissolved in MeOH (100 mL). The mixture was heated at 60° C. for 1 h then concentrated to afford a yellow solid which was dried in a vacuum oven at 50° C. for 48 hrs to provide the title compound as a yellow solid. MS (ESI): Mass calcd. for C₂₀H₁₉DN₁₀O₂, 433.4; m/z found, 434.2 [M+H]⁺. ¹H NMR (400 MHz, DMSO-d₆) δ 10.02 (s, 1H), 9.01 (s, 1H), 8.54-8.50 (m, 1H), 8.44-8.37 (m, 1H), 8.35-8.30 (m, 1H), 7.90-7.87 (m, 1H), 7.70-7.67 (m, 1H), 6.81-6.77 (m, 1H), 6.43 (s, 1H), 3.84 (s, 3H), 3.58-3.53 (m, 2H), 2.89 (s, 3H), 2.88-2.79 (m, 1H), 2.40-2.29 (m, 1H).

### Example 194: (R)-3-(3-(6-(2-((6,7-Dihydro-5H-pyrazolo[5,1-b][1,3]oxazin-3-yl)amino)pyrimidin-4-yl)pyridin-2-yl)isoxazol-5-yl)-3-hydroxy-1-methylpyrrolidin-2-one

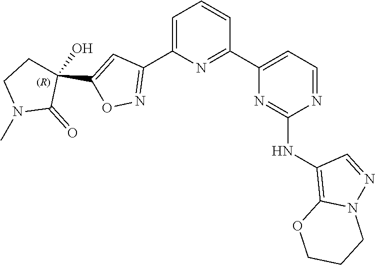

A mixture of (R)-3-(3-(6-(2-chloropyrimidin-4-yl)pyridin-2-yl)isoxazol-5-yl)-3-hydroxy-1-methylpyrrolidin-2-one (200 mg, 0.538 mmol, Intermediate 31), 6,7-dihydro-5H-pyrazolo[5,1-b][1,3]oxazin-3-amine (100 mg, 0.719 mmol) and Cs₂CO₃ (530 mg, 1.63 mmol) in 1,4-dioxane (8 mL) was purged with N₂ three times and then Josiphos-Pd-G3 (100 mg, 0.108 mmol) was added. The resulting mixture was purged with N₂ three times, then heated at 110° C. for 16 hrs. The mixture was cooled to room temperature and extracted with EtOAc (3×15 mL). The combined organic layers were washed with brine (8 mL), dried over anhydrous Na₂SO₄, filtered and concentrated. The residue was purified by reverse-phase HPLC (Boston Prime C18 column, 5 μm, 30×150 mm; 30-60% ACN/H₂O (with 0.05% aqueous NH₃ and 10 mM NH₄HCO₃)) to afford the title compound as a yellow solid (37 mg, 14%). MS (ESI): Mass calcd. for C₂₃H₂₂N₈O₄, 474.5; m/z found, 475.2 [M+H]⁺. ¹H NMR (400 MHz, DMSO-d₆) δ 8.65-8.47 (m, 2H), 8.36 (br s, 1H), 8.22-8.07 (m, 2H), 7.75-7.68 (m, 1H), 7.40 (br s, 1H), 7.16 (s, 1H), 6.78 (s, 1H), 4.33-4.25 (m, 2H), 4.17-4.07 (m, 2H), 3.55-3.40 (m, 2H), 2.85 (s, 3H), 2.70-2.58 (m, 1H), 2.35-2.24 (m, 1H), 2.24-2.14 (m, 2H).

### Example 195: (R)-3-Hydroxy-1-methyl-3-(3-(6-(2-(((*R)-4-methyl-6,7-dihydro-4H-pyrazolo[5,1-c][1,4]oxazin-3-yl)amino)pyrimidin-4-yl)pyridin-2-yl)isoxazol-5-yl)pyrrolidin-2-one

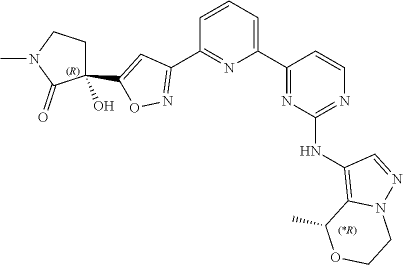

The title compound was prepared using conditions analogous to those described in Example 194 using (*R)-4-methyl-6,7-dihydro-4H-pyrazolo[5,1-c][1,4]oxazin-3-amine (Intermediate 70) in place of 6,7-dihydro-5H-pyrazolo[5,1-b][1,3]oxazin-3-amine and Xantphos-Pd-G3 in place of Josiphos-Pd-G3. The residue was purified sequentially by silica gel chromatography (0-10% EtOH/EtOAc) and then SFC (DAICEL CHIRALCEL OJ, 10 μm, 30×250 mm, isocratic elution: 35% EtOH (0.1% of aqueous NH₃), 65% CO₂) to provide the title compound as a yellow solid (30 mg, 23%). MS (ESI): Mass calcd. for C₂₄H₂₄N₈O₄, 488.5; m/z found, 489.2 [M+H]⁺. ¹H NMR (400 MHz, DMSO-d₆) δ 8.99 (br s, 1H), 8.58 (d, J=4.8 Hz, 1H), 8.35 (br s, 1H), 8.22-8.11 (m, 2H), 7.75 (d, J=4.8 Hz, 1H), 7.56 (br s, 1H), 7.15 (s, 1H), 6.77 (br s, 1H), 4.96 (q, J=6.4 Hz, 1H), 4.27-4.16 (m, 1H), 4.15-4.05 (m, 2H), 4.01-3.88 (m, 1H), 3.55-3.41 (m, 2H), 2.85 (s, 3H), 2.66-2.57 (m, 1H), 2.33-2.25 (m, 1H), 1.30 (d, J=6.4 Hz, 3H).

### Example 196: (R)-3-Hydroxy-1-methyl-3-(3-(6-(2-((2-methylpyridin-4-yl)amino)pyrimidin-4-yl)pyridin-2-yl)isoxazol-5-yl)pyrrolidin-2-one

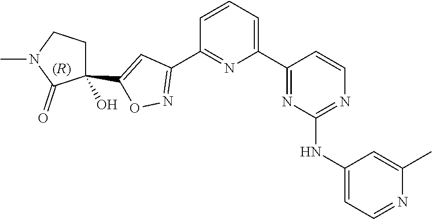

The title compound was prepared using conditions analogous to those described in Example 194 using 2-methylpyridin-4-amine in place of 6,7-dihydro-5H-pyrazolo[5,1-b][1,3]oxazin-3-amine and Xantphos-Pd-G3 in place of Josiphos-Pd-G3. The reaction mixture was concentrated, purified directly by silica gel chromatography (0-100% EtOAc/petroleum ether) and then triturated with MeOH (8 mL), filtering the suspension, washing the filter cake with MeOH (15 mL) and then drying the solid under high vacuum to afford the title compound as a yellow solid (52 mg, 22%). MS (ESI): Mass calcd. for C₂₃H₂₁N₇O₃, 443.5; m/z found, 444.2 [M+H]⁺. ¹H NMR (400 MHz, DMSO-d₆) δ 10.23 (s, 1H), 8.83-8.79 (m, 1H), 8.54-8.48 (m, 1H), 8.32-8.18 (m, 3H), 8.03-8.00 (m, 1H), 7.75-7.71 (m, 1H), 7.70 (s, 1H), 7.21 (s, 1H), 6.81 (s, 1H), 3.55-3.41 (m, 2H), 2.86 (s, 3H), 2.70-2.59 (m, 1H), 2.45 (s, 3H), 2.35-2.27 (m, 1H).

### Example 197: (R)-3-Hydroxy-1-methyl-3-(3-(6-(2-((2-methylpyridin-3-yl)amino)pyrimidin-4-yl)pyridin-2-yl)isoxazol-5-yl)pyrrolidin-2-one

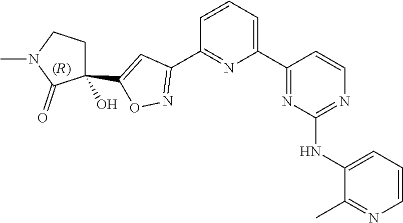

The title compound was prepared using conditions analogous to those described in Example 194 using 2-methylpyridin-3-amine in place of 6,7-dihydro-5H-pyrazolo[5,1-b][1,3]oxazin-3-amine and Xantphos-Pd-G3 in place of Josiphos-Pd-G3. The residue was purified by reverse-phase HPLC (Phenomenex C18 column, 3 μm, 40×80 mm; 37-67% ACN/H₂O (with 0.05% NH₃ and 10 mM NH₄HCO₃)) to provide the title compound as a white solid (70 mg, 25%). MS (ESI): Mass calcd. for C₂₃H₂₁N₇O₃, 443.5; m/z found, 444.2 [M+H]⁺. 1H NMR (400 MHz, DMSO-d₆) δ 9.19 (s, 1H), 8.67-8.61 (m, 1H), 8.34-8.29 (m, 1H), 8.28-8.24 (m, 1H), 8.18-8.12 (m, 2H), 8.02-7.97 (m, 1H), 7.88-7.84 (m, 1H), 7.31-7.25 (m, 1H), 7.17 (s, 1H), 6.77 (s, 1H), 3.54-3.41 (m, 2H), 2.85 (s, 3H), 2.66-2.59 (m, 1H), 2.49 (br. s, 3H), 2.34-2.26 (m, 1H).

### Example 198: (*R)-3-(3-(6-(2-((5-(Difluoromethyl)-1-methyl-1H-pyrazol-4-yl)amino)pyrimidin-4-yl)pyridin-2-yl)isoxazol-5-yl)-3-hydroxy-1-methylpyrrolidin-2-one

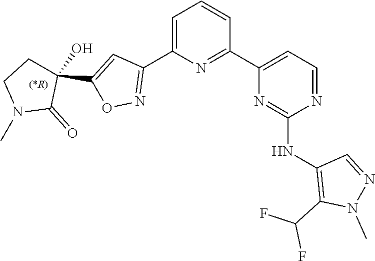

The title compound (23 mg, 13%) was prepared using conditions analogous to those described in Example 194 using 5-(difluoromethyl)-1-methyl-1H-pyrazol-4-amine hydrochloride in place of 6,7-dihydro-5H-pyrazolo[5,1-b][1,3]oxazin-3-amine. MS (ESI): Mass calcd. for C₂₂H₂O F₂N₈O₃, 482.4; m/z found, 483.2 [M+H]⁺. ¹H NMR (400 MHz, DMSO-d₆) δ 9.47 (s, 1H), 8.68-8.62 (m, 1H), 8.43-8.38 (m, 1H), 8.24-8.11 (m, 2H), 7.92 (s, 1H), 7.88-7.82 (m, 1H), 7.51-7.21 (m, 1H), 7.18 (s, 1H), 6.80 (s, 1H), 3.96 (s, 3H), 3.54-3.45 (m, 2H), 2.85 (s, 3H), 2.69-2.58 (m, 1H), 2.36-2.24 (m, 1H).

### Example 199: (*R)-3-Hydroxy-3-(3-(6-(2-((1-(2-methoxyethyl)-1H-pyrazol-3-yl)amino)pyrimidin-4-yl)pyridin-2-yl)isoxazol-5-yl)-1-methylpyrrolidin-2-one

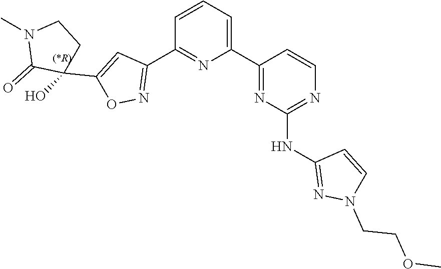

The title compound was prepared using conditions analogous to those described in Example 194 using 1-(2-methoxyethyl)-1H-pyrazol-4-amine in place of 6,7-dihydro-5H-pyrazolo[5,1-b][1,3]oxazin-3-amine and Xantphos-Pd-G3 in place of Josiphos-Pd-G3. The residue was dissolved in DMF (5 mL), Biotage® MP-TMT (2.2 mL) was added and the resulting mixture was stirred at room temperature for 16 hrs. The mixture was filtered and the filtrate concentrated to provide a yellow oil. The oil was purified sequentially by reverse-phase HPLC (Xtimate C18 column, 5 μm, 30×150 mm; 20-50% ACN/H₂O (with 0.2% formic acid)) and then trituration with MeCN (3 mL). A light-yellow precipitate formed, which was collected by filtration and dried under high vacuum to provide the title compound as a light-yellow solid (63 mg, 24%). MS (ESI): Mass calcd. for C₂₃H₂₄N₈O₄, 476.5; m/z found, 477.2 [M+H]⁺. ¹H NMR (400 MHz, DMSO-d₆) δ 9.96 (s, 1H), 8.64 (d, J=5.2 Hz, 1H), 8.48-8.44 (m, 1H), 8.23-8.12 (m, 2H), 7.82 (d, J=5.2 Hz, 1H), 7.65-7.62 (m, 1H), 7.17 (s, 1H), 6.88-6.76 (m, 1H), 6.75-6.70 (m, 1H), 4.18 (t, J=5.2 Hz, 2H), 3.68 (t, J=5.6 Hz, 2H), 3.52-3.44 (m, 2H), 3.24 (s, 3H), 2.84 (s, 3H), 2.65-2.58 (m, 1H), 2.35-2.24 (m, 1H).

### Example 200: (R)-3-Hydroxy-1-methyl-3-(5-(6-(2-((1-methyl-1H-pyrazol-3-yl)amino)pyrimidin-4-yl-6-d)pyridin-2-yl)isoxazol-3-yl)pyrrolidin-2-one

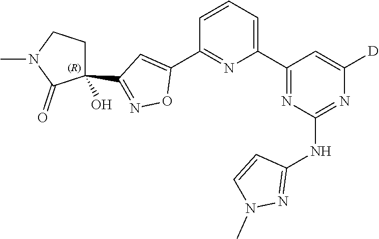

Step A. (R)-3-(5-(6-(2-Chloropyrimidin-4-yl-6-d)pyridin-2-yl)isoxazol-3-yl)-3-hydroxy-1-methylpyrrolidin-2-one. A mixture of (R)-3-(5-(6-bromopyridin-2-yl)isoxazol-3-yl)-3-hydroxy-1-methylpyrrolidin-2-one (21.0 g, 62.1 mmol, Intermediate 52) and Pd(PPh₃)₄ (458 mg, 1.24 mmol) in THF (210 mL) was heated to 60° C. Then, (2-chloropyrimidin-4-yl-6-d)zinc(II) chloride (217 mL, 0.715 M in THF, Intermediate 50) was added and the resulting mixture was heated at 60° C. for 1 h. After that time, the reaction was poured into 0° C. saturated aqueous NH₄Cl (200 mL) and the mixture was extracted with EtOAc (3×200 mL). The combined organic layers were washed with brine (200 mL), dried over anhydrous Na₂SO₄, filtered and concentrated. The residue was triturated with MeOH (100 mL) at 25° C. for 12 hrs, then filtered and the solids dissolved in DCM (1 L). The solution was stirred at 25° C. for 12 hrs, then filtered and the filtrate was concentrated down to 300 mL. Then, petroleum ether (300 mL) was added and the mixture was stirred for 15 min. The mixture was filtered and the filtrate was concentrated to provide the title compound as a brown solid (20.1 g, 83%). ¹H NMR (400 MHz, DMSO-d₆) δ 8.53 (s, 1H), 8.46 (d, J=7.6 Hz, 1H), 8.09-8.30 (m, 2H), 7.40 (s, 1H), 6.59 (s, 1H), 3.39-3.49 (m, 2H), 2.81 (s, 3H), 2.64-2.73 (m, 1H), 2.21-2.34 (m, 1H). MS (ESI⁺): m/z=373.0.

Step B. (R)-3-Hydroxy-1-methyl-3-(5-(6-(2-((1-methyl-1H-pyrazol-3-yl)amino)pyrimidin-4-yl-6-d)pyridin-2-yl)isoxazol-3-yl)pyrrolidin-2-one. To a solution of (R)-3-(5-(6-(2-chloropyrimidin-4-yl-6-d)pyridin-2-yl)isoxazol-3-yl)-3-hydroxy-1-methylpyrrolidin-2-one (20.1 g, 53.9 mmol) in THF (400 mL) was added 1-methyl-1H-pyrazol-3-amine (10.5 g, 108 mmol) and the resulting mixture was cooled to −40° C. Then, LiHMDS (215.7 mL, 1 M in THF) was added and the mixture was stirred at −40° C. for 3 hrs. After that time, the reaction was poured into 0° C. water (1 L) and the pH of the solution was adjusted to pH 10 by the addition of 2 M aqueous HCl. The mixture was concentrated down to 1 L and a solid precipitated out. The mixture was filtered and the filtrate was extracted with DCM/MeOH (10/1, 3×500 mL). The combined organic layers and filter cake were concentrated to afford a yellow solid which was purified by silica gel chromatography (0-10% MeOH/DCM) to afford a yellow solid. To the solid was added DMF (190 mL) and the mixture was heated to 50° C. Then, MeOH (48 mL) and THF (48 mL) were added sequentially and the resulting mixture stood at room temperature for 16 hrs. After that time, water (600 mL) was added and the mixture stirred at room temperature for 15 hrs. The mixture was filtered and the filter cake was washed with 0° C. MeOH (3×100 mL). The filtrate was concentrated to give a yellow solid which was triturated with MeOH (100 mL) at 25° C. for 16 hrs. The solid was then dissolved in DCM (280 mL), MeOH (100 mL) was added and the mixture was concentrated to 150 mL. Then, MeOH (300 mL) was added and the mixture stirred at room temperature for 15 min. The mixture was filtered and the filtrate concentrated to afford a yellow solid. The solid was dissolved in MeOH (100 mL) and the solution was heated at 60° C. for 1 h, then concentrated. To the solid residue was added MeOH (100 mL) and the mixture was heated at 60° C. for 1 h, then concentrated. The solid obtained was dried in a vacuum oven at 50° C. for 48 hrs to provide the title compound as a yellow solid (12.0 g, 51%). MS (ESI): Mass calcd. for C₂₁H₁₉DN₈O₃, 433.4; m/z found, 434.2 [M+H]⁺. ¹H NMR (400 MHz, CD₃OD) δ 8.54-8.49 (m, 1H), 8.14-8.07 (m, 1H), 8.04-8.00 (m, 1H), 7.88 (s, 1H), 7.52-7.49 (m, 1H), 7.31 (s, 1H), 6.75-6.71 (m, 1H), 3.83 (s, 3H), 3.57-3.52 (m, 2H), 2.93 (s, 3H), 2.90-2.83 (m, 1H), 2.42-2.33 (m, 1H).

### Example 201 and Example 202: (R)-3-(3-(6-(2-((1-((3S,4S)-3-Fluoro-1-methylpiperidin-4-yl)-1H-pyrazol-4-yl)amino)pyrimidin-4-yl)pyridin-2-yl)isoxazol-5-yl)-3-hydroxy-1-methylpyrrolidin-2-one and (R)-3-(3-(6-(2-((1-((3R,4R)-3-fluoro-1-methylpiperidin-4-yl)-1H-pyrazol-4-yl)amino)pyrimidin-4-yl)pyridin-2-yl)isoxazol-5-yl)-3-hydroxy-1-methylpyrrolidin-2-one

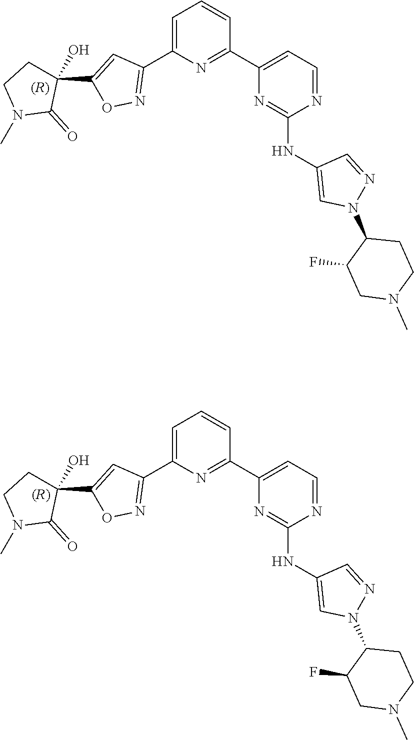

A mixture of 1-(3-fluoro-1-methylpiperidin-4-yl)-1H-pyrazol-4-amine (940 mg, 4.74 mmol, Intermediate 72), (R)-3-hydroxy-1-methyl-3-(3-(6-(2-(methylsulfonyl)pyrimidin-4-yl)pyridin-2-yl)isoxazol-5-yl)pyrrolidin-2-one (400 mg, 0.963 mmol, Intermediate 13) and TsOH (830 mg, 4.82 mmol) in 1,4-dioxane (3 mL) was heated at 110° C. for 12 hrs. The mixture was cooled to room temperature, diluted with water (10 mL) and extracted with DCM (3×5 mL). The combined organic layers were washed with brine, dried over anhydrous Na₂SO₄, filtered and concentrated. The residue was purified by reverse-phase HPLC (Xtimate C18 column, 5 μm, 30×150 mm; 20-50% ACN/H₂O (with 0.05% NH₃ and 10 mM NH₄HCO₃)) to afford a mixture of diastereomers as a yellow solid. The diastereomers were separated by chiral SFC (DAICEL CHIRALCEL OD-H, 5 μm, 30×250 mm, isocratic elution: 55% IPA (0.1% of 25% aqueous NH₃), 45% CO₂) to provide Example 201 as the first-eluting diastereomer (80 mg, 16%). The second-eluting diastereomer was further purified by SFC (DAICEL CHIRALCEL OD, 10 μm, 30×250 mm, isocratic elution: 50% MeOH (0.1% of 25% aqueous NH₃), 50% CO₂) to provide Example 202 as the second-eluting diastereomer (85 mg, 81%). Example 201: MS (ESI): Mass calcd. for C₂₆H₂₈FN₉O₃, 533.6; m/z found, 534.2 [M+H]⁺. ¹H NMR (400 MHz, DMSO-d₆) δ 9.68 (s, 1H), 8.64 (d, J=4.8 Hz, 1H), 8.53-8.46 (m, 1H), 8.25-8.14 (m, 2H), 8.08 (s, 1H), 7.81-7.67 (m, 2H), 7.18 (s, 1H), 6.79 (s, 1H), 4.99-4.76 (m, 1H), 4.44-4.26 (m, 1H), 3.53-3.41 (m, 2H), 3.27-3.19 (m, 1H), 2.87-2.82 (m, 4H), 2.66-2.58 (m, 1H), 2.33-2.26 (m, 4H), 2.15-1.97 (m, 4H). Example 202: MS (ESI): Mass calcd. for C₂₆H₂₈FN₉O₃, 533.6; m/z found, 534.2 [M+H]⁺. ¹H NMR (400 MHz, DMSO-d₆) δ 9.68 (s, 1H), 8.64 (d, J=4.8 Hz, 1H), 8.54-8.44 (m, 1H), 8.25-8.14 (m, 2H), 8.08 (s, 1H), 7.82-7.66 (m, 2H), 7.18 (s, 1H), 6.79 (s, 1H), 5.01-4.77 (m, 1H), 4.45-4.27 (m, 1H), 3.54-3.42 (m, 2H), 3.28-3.21 (m, 1H), 2.91-2.81 (m, 4H), 2.66-2.58 (m, 1H), 2.35-2.29 (m, 4H), 2.21-2.06 (m, 3H), 2.06-1.96 (m, 1H).

### Example 203: (R)-3-Hydroxy-1-methyl-3-(3-(6-(2-((1-((*S)-1-methylpyrrolidin-3-yl)-1H-pyrazol-4-yl)amino)pyrimidin-4-yl)pyridin-2-yl)isoxazol-5-yl)pyrrolidin-2-one

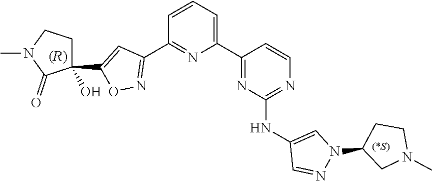

Step A. tert-Butyl 3-(4-((4-(6-(5-((R)-3-hydroxy-1-methyl-2-oxopyrrolidin-3-yl)isoxazol-3-yl)pyridin-2-yl)pyrimidin-2-yl)amino)-1H-pyrazol-1-yl)pyrrolidine-1-carboxylate. A solution of tert-butyl 3-(4-amino-1H-pyrazol-1-yl)pyrrolidine-1-carboxylate (1.22 g, 4.84 mmol), (R)-3-hydroxy-1-methyl-3-(3-(6-(2-(methylsulfonyl)pyrimidin-4-yl)pyridin-2-yl)isoxazol-5-yl)pyrrolidin-2-one (200 mg, 0.481 mmol, Intermediate 13) and TsOH (420 mg, 2.44 mmol) in 1,4-dioxane (8 mL) was heated at 110° C. for 12 hrs. Then, the mixture was diluted with water (10 mL) and extracted with DCM (3×5 mL). The combined organic layers were washed with brine, dried over anhydrous Na₂SO₄, filtered and concentrated. The residue was purified by reverse-phase HPLC (Xtimate C18 column, 10 μm, 40×150 mm; 30-60% ACN/H₂O (with 0.05% aqueous NH₃ and 10 mM NH₄HCO₃)) to afford the title compound as a yellow solid (300 mg, 23.3%). MS (ESI⁺): m/z=588.2.

Step B. (3R)-3-Hydroxy-1-methyl-3-(3-(6-(2-((1-(pyrrolidin-3-yl)-1H-pyrazol-4-yl)amino)pyrimidin-4-yl)pyridin-2-yl)isoxazol-5-yl)pyrrolidin-2-one trifluoroacetate. To a solution of tert-butyl 3-(4-((4-(6-(5-((R)-3-hydroxy-1-methyl-2-oxopyrrolidin-3-yl)isoxazol-3-yl)pyridin-2-yl)pyrimidin-2-yl)amino)-1H-pyrazol-1-yl)pyrrolidine-1-carboxylate (120 mg, 0.204 mmol) in DCM (6 mL) at 0° C. was added TFA (2 mL) dropwise. The resulting mixture was stirred at room temperature for 1 h, then concentrated to afford a brown oil (150 mg) which was used without further purification. MS (ESI⁺): m/z=488.2.

Step C. (R)-3-Hydroxy-1-methyl-3-(3-(6-(2-((1-((*S)-1-methylpyrrolidin-3-yl)-1H-pyrazol-4-yl)amino)pyrimidin-4-yl)pyridin-2-yl)isoxazol-5-yl)pyrrolidin-2-one. To a solution of (3R)-3-hydroxy-1-methyl-3-(3-(6-(2-((1-(pyrrolidin-3-yl)-1H-pyrazol-4-yl)amino)pyrimidin-4-yl)pyridin-2-yl)isoxazol-5-yl)pyrrolidin-2-one trifluoroacetate (100 mg, 0.167 mmol) and formaldehyde (150 mg, 1.65 mmol, 33% in water) in MeOH (1 mL) was added NaOAc (40 mg, 0.488 mmol). The resulting mixture was stirred at room temperature for 10 min, then NaBH₃CN (4.00 mg, 0.064 mmol) was added and the mixture was stirred at room temperature for 12 hrs. The reaction mixture was diluted with water (10 mL) and extracted with DCM (3×5 mL). The combined organic layers were washed with brine, dried over anhydrous Na₂SO₄, filtered and concentrated. The residue was purified by reverse-phase HPLC (Xtimate C18 column, 5 μm, 30×150 mm; 30-60% ACN/H₂O (with 0.05% aqueous NH₃ and 10 mM NH₄HCO₃)) to provide a mixture of diastereomers. The diastereomers were separated by chiral SFC (DAICEL CHIRALPAK AD, 10 μm, 30×250 mm, isocratic elution: 60% IPA (0.1% of 25% aqueous NH₃), 40% CO₂) to provide Example 203 as the second-eluting diastereomer (6 mg, 14%). MS (ESI): Mass calcd. for C₂₅H₂₇N₉O₃, 501.5; m/z found, 502.2 [M+H]⁺. ¹H NMR (400 MHz, DMSO-d₆) δ 9.68 (s, 1H), 8.67-8.61 (m, 1H), 8.52-8.45 (m, 1H), 8.28-8.09 (m, 3H), 7.81-7.75 (m, 1H), 7.81-7.75 (m, 1H), 7.58 (s, 1H), 7.18 (s, 1H), 6.90-6.70 (m, 1H), 4.96-4.84 (m, 1H), 3.54-3.38 (m, 5H), 2.85 (s, 3H), 2.81-2.74 (m, 2H), 2.70-2.57 (m, 2H), 2.33 (s, 3H), 2.09-1.98 (m, 1H).

### Example 204: (R)-3-(3-(6-(2-((1-(2-Aminoethyl)-1H-pyrazol-3-yl)amino)pyrimidin-4-yl)pyridin-2-yl)isoxazol-5-yl)-3-hydroxy-1-methylpyrrolidin-2-one

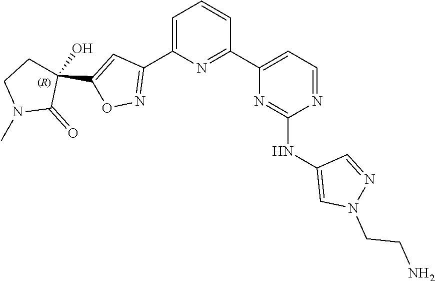

Step A. tert-Butyl (R)-(2-(3-((4-(6-(5-(3-hydroxy-1-methyl-2-oxopyrrolidin-3-yl)isoxazol-3-yl)pyridin-2-yl)pyrimidin-2-yl)amino)-1H-pyrazol-1-yl)ethyl)carbamate. To a mixture of (R)-3-(3-(6-(2-chloropyrimidin-4-yl)pyridin-2-yl)isoxazol-5-yl)-3-hydroxy-1-methylpyrrolidin-2-one (20 mg, 0.054 mmol, Intermediate 31), tert-butyl (2-(3-amino-1H-pyrazol-1-yl)ethyl)carbamate (16 mg, 0.071 mmol, Intermediate 71) and Cs₂CO₃ (39 mg, 0.12 mmol) in 1,4-dioxane (1 mL) were added Xantphos (4 mg, 0.007 mmol) and Pd(OAc)₂ (1.5 mg, 0.007 mmol). The resulting mixture was sparged with Ar for 5 min and then heated at 100° C. for 12 hrs. The reaction mixture was diluted with water (10 mL) and extracted with DCM (3×5 mL). The combined organic layers were washed with brine, dried over anhydrous Na₂SO₄, filtered and concentrated. The residue was purified by reverse-phase HPLC (Xtimate C18 column, 5 μm, 30×150 mm; 33-63% ACN/H₂O (with 0.05% aqueous NH₃ and 10 mM NH₄HCO₃)) to afford the title compound as a yellow solid (40 mg, 79%). MS (ESI⁺): m/z=562.2.

Step B. (R)-3-(3-(6-(2-((1-(2-Aminoethyl)-1H-pyrazol-3-yl)amino)pyrimidin-4-yl)pyridin-2-yl)isoxazol-5-yl)-3-hydroxy-1-methylpyrrolidin-2-one. To a solution of tert-butyl (R)-(2-(3-((4-(6-(5-(3-hydroxy-1-methyl-2-oxopyrrolidin-3-yl)isoxazol-3-yl)pyridin-2-yl)pyrimidin-2-yl)amino)-1H-pyrazol-1-yl)ethyl)carbamate (30 mg, 0.018 mmol) in DCM (3 mL) at 0° C. was added TFA (1.0 mL, 13 mmol) dropwise and the resulting mixture was stirred at room temperature for 1 h. After that time, the mixture was concentrated and purified by reverse-phase HPLC (Xtimate C18 column, 5 μm, 30×150 mm; 23-53% ACN/H₂O (with 0.05% NH₃ in water and 10 mM NH₄HCO₃)) to afford the title compound as an off-white solid (6 mg, 24%). MS (ESI): Mass calcd. for C₂₂H₂₃N₉O₃, 461.5; m/z found, 462.2 [M+H]⁺. ¹H NMR (400 MHz, DMSO-d₆) δ 9.94 (s, 1H), 8.64 (d, J=5.2 Hz, 1H), 8.49-8.45 (m, 1H), 8.24-8.13 (m, 2H), 7.82 (d, J=5.2 Hz, 1H), 7.67-7.62 (m, 1H), 7.17 (s, 1H), 6.87-6.71 (m, 2H), 4.03-3.97 (m, 2H), 3.53-3.39 (m, 4H), 2.94-2.88 (m, 2H), 2.85 (s, 3H), 2.69-2.58 (m, 1H), 2.34-2.25 (m, 1H).

### Example 205: (R)-3-(1-(6-(2-((3-(2,2-Difluoroethoxy)-1-methyl-1H-pyrazol-4-yl)amino)pyrimidin-4-yl)pyridin-2-yl)-1H-1,2,3-triazol-4-yl)-3-hydroxy-1-methylpyrrolidin-2-one

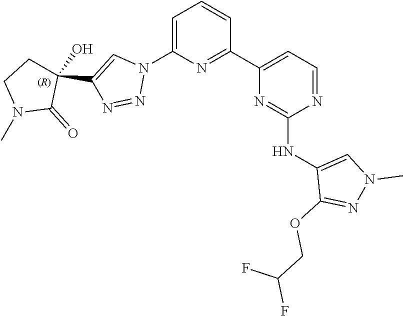

A flask containing (R)-3-(1-(6-(2-chloropyrimidin-4-yl)pyridin-2-yl)-1H-1,2,3-triazol-4-yl)-3-hydroxy-1-methylpyrrolidin-2-one (100 mg, 0.269 mmol, Intermediate 32), 3-(2,2-difluoroethoxy)-1-methyl-1H-pyrazol-4-amine (80.0 mg, 0.348 mmol), t-BuBrettphos-Pd-G3 (23 mg, 0.027 mmol) and t-BuBrettphos (13 mg, 0.027 mmol) was evacuated and refilled with N₂ three times. Then, anhydrous THF (5 mL) and LiHMDS (0.8 mL, 0.8 mmol, 1 M in THF) were added sequentially under a flow of N₂. The flask was evacuated and refilled with N₂ three times, then the mixture was heated at 70° C. for 16 hrs. The mixture was cooled to room temperature, diluted with EtOAc (200 mL), washed with brine (3×30 mL), dried over anhydrous Na₂SO₄, filtered and concentrated. The residue was purified by reverse-phase HPLC (Xtimate C18 column, 5 μm, 25×150 mm; 35-65% ACN/H₂O (with 0.05% NH₃ in water and 10 mM NH₄HCO₃)) to afford, after lyophilization, the title compound as a yellow solid (35 mg, 25%). MS (ESI): Mass calcd. for C₂₂H₂₂F₂N₁₀O₃, 512.5; m/z found, 513.2 [M+H]⁺. ¹H NMR (400 MHz, DMSO-d₆) δ 8.94 (s, 1H), 8.73 (s, 1H), 8.59 (d, J=4.8 Hz, 1H), 8.46-8.15 (m, 3H), 7.92-7.67 (m, 2H), 6.54-6.17 (m, 2H), 4.40 (dt, J=3.2, 14.4 Hz, 2H), 3.74 (s, 3H), 3.49 (t, J=6.4 Hz, 2H), 2.91-2.71 (m, 4H), 2.37-2.21 (m, 1H).

### Example 206: (R)-3-Hydroxy-1-methyl-3-(1-(6-(2-((1-methyl-1H-pyrazol-4-yl)amino)pyrimidin-4-yl)pyridin-2-yl)-1H-1,2,3-triazol-4-yl)pyrrolidin-2-one

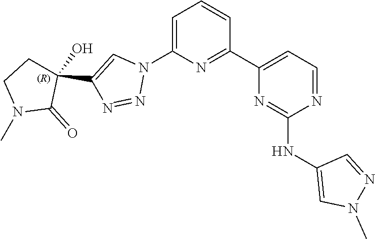

A flask containing (R)-3-(1-(6-bromopyridin-2-yl)-1H-1,2,3-triazol-4-yl)-3-hydroxy-1-methylpyrrolidin-2-one (200 mg, 0.591 mmol, Intermediate 51), N-(1-methyl-1H-pyrazol-4-yl)-4-(tributylstannyl)pyrimidin-2-amine (280 mg, 0.603 mmol, Intermediate 76), Pd(PPh₃)₄ (70 mg, 0.051 mmol) and TEA (165 μL, 0.061 mmol) in xylenes (5 mL) was evacuated and refilled with N₂ three times. The resulting mixture was heated at 120° C. for 16 hrs, then cooled to room temperature and filtered. The filter cake was triturated with MTBE (10 mL) and ACN (10 mL) to provide the title compound as a yellow solid (210 mg, 81%). MS (ESI): Mass calcd. for C₂₀H₂₀N₁₀O₂, 432.4; m/z found, 433.1 [M+H]⁺. ¹H NMR (400 MHz, DMSO-d₆) δ 9.70 (s, 1H), 8.95 (s, 1H), 8.66 (d, J=4.8 Hz, 1H), 8.54-8.43 (m, 1H), 8.35 (t, J=8.0 Hz, 1H), 8.30-8.22 (m, 1H), 7.99 (s, 1H), 7.76 (d, J=4.8 Hz, 1H), 7.60 (s, 1H), 6.36 (s, 1H), 3.87 (s, 3H), 3.50 (t, J=4.8 Hz, 2H), 2.89-2.74 (m, 4H), 2.35-2.20 (m, 1H).

### Example 207: (R)-3-Hydroxy-1-methyl-3-(3-(6-(6-methyl-2-((1-methyl-1H-pyrazol-4-yl)amino)pyrimidin-4-yl)pyridin-2-yl)isoxazol-5-yl)pyrrolidin-2-one

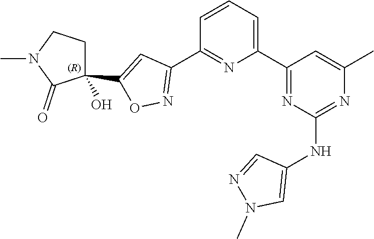

To a solution of (R)-3-(3-(6-chloropyridin-2-yl)isoxazol-5-yl)-3-hydroxy-1-methylpyrrolidin-2-one (200 mg, 0.681 mmol, Intermediate 56) in toluene (10 mL) were added 4-methyl-N-(1-methyl-1H-pyrazol-4-yl)-6-(tributylstannyl)pyrimidin-2-amine (390 mg, 0.815 mmol, Intermediate 57), TEA (0.2 mL, 1.44 mmol) and Pd(PPh₃)₄ (80 mg, 0.069 mmol). The resulting mixture was purged with Ar three times and then heated at 120° C. for 10 hrs. After that time, saturated aqueous KF (20 mL) was added and the mixture was stirred at room temperature for 1 h. The precipitate was removed by filtration and the filtrate extracted with EtOAc (3×20 mL). The combined organic layers were washed with brine, dried over anhydrous Na₂SO₄, filtered and concentrated. The residue was purified sequentially by reverse-phase HPLC (Xtimate C18 column, 5 μm, 30×150 mm; 30-60% ACN/H₂O with 0.05% NH₃ in water and 10 mM NH₄HCO₃) and then SFC (DAICEL CHIRALPAK AS, 10 m, 30×250 mm, isocratic elution: 60% EtOH (0.1% of aqueous NH₃), 40% CO₂) to afford the title compound as a yellow solid (71 mg, 22%). MS (ESI): Mass calcd. for C₂₂H₂₂N₈O₃, 446.5; m/z found, 447.2 [M+H]⁺. ¹H NMR (400 MHz, DMSO-d₆) δ 9.57 (s, 1H), 8.55-8.42 (m, 1H), 8.24-8.12 (m, 2H), 7.98 (s, 1H), 7.66 (s, 1H), 7.59 (s, 1H), 7.19 (s, 1H), 6.81 (br s, 1H), 3.86 (s, 3H), 3.54-3.43 (m, 2H), 3.39-3.36 (m, 3H), 2.86 (s, 3H), 2.69-2.58 (m, 1H), 2.36-2.25 (m, 1H).

### Example 208: (R)-3-Hydroxy-1-methyl-3-(3-(6-(5-methyl-2-((1-methyl-1H-pyrazol-4-yl)amino)pyrimidin-4-yl)pyridin-2-yl)isoxazol-5-yl)pyrrolidin-2-one

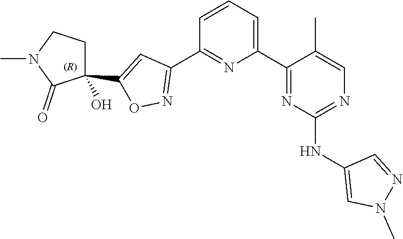

To a solution of (R)-3-(3-(6-bromopyridin-2-yl)isoxazol-5-yl)-3-hydroxy-1-methylpyrrolidin-2-one (210 mg, 0.621 mmol, Intermediate 13 Step A) in 1,4-dioxane (8 mL) were added 5-methyl-N-(1-methyl-1H-pyrazol-4-yl)-4-(trimethylstannyl)pyrimidin-2-amine (230 mg, 0.653 mmol, Intermediate 55) and Pd(P(Cy)₃)₂C₁₂ (900 mg, 1.22 mol) and the resulting mixture was purged with Ar ten times then heated at 105° C. for 40 hrs. After that time, saturated aqueous KF (50 mL) was added and the mixture was stirred at room temperature for 1 h. The precipitate was removed by filtration and the filtrate extracted with EtOAc (3×50 mL). The combined organic layers were washed with brine, dried over anhydrous Na₂SO₄, filtered and concentrated. The residue was purified sequentially by reverse-phase HPLC (Boston Prime C18 column, 5 μm, 30×150 mm; 30-60% ACN/H₂O with 0.05% NH₃ and 10 mM NH₄HCO₃) and then SFC (DAICEL CHIRALCEL OJ, 10 μm, 30×250 mm, isocratic elution: 40% EtOH (0.1% NH₃ in H₂O), 60% CO₂) to afford the title compound as a yellow solid (20 mg, 7%). MS (ESI): Mass calcd. for C₂₂H₂₂N₈O₃, 446.5; m/z found, 447.2 [M+H]⁺. ¹H NMR (400 MHz, DMSO-d₆) δ 9.46 (s, 1H), 8.45 (s, 1H), 8.26-8.09 (m, 3H), 7.89 (s, 1H), 7.52 (s, 1H), 7.01 (s, 1H), 6.81 (s, 1H), 3.82 (s, 3H), 3.53-3.41 (m, 2H), 2.85 (s, 3H), 2.65-2.55 (m, 1H), 2.46 (s, 3H), 2.33-2.23 (m, 1H).

### Example 209: (R)-3-Hydroxy-1-methyl-3-(3-(6-(2-((1-methyl-1H-pyrazol-3-yl)amino)pyrimidin-4-yl-6-d)pyridin-2-yl)isoxazol-5-yl)pyrrolidin-2-one

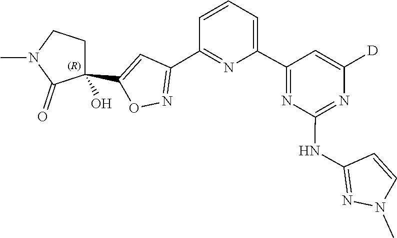

To a solution of (R)-3-(3-(6-(2-chloropyrimidin-4-yl-6-d)pyridin-2-yl)isoxazol-5-yl)-3-hydroxy-1-methylpyrrolidin-2-one (21.5 g, 57.6 mmol, Intermediate 53) in THF (215 mL) was added 1-methyl-1H-pyrazol-3-amine (7.69 g, 69.2 mmol), and the resulting solution was cooled to 0-10° C. Then, LiHMDS (230 mL, 230 mmol, 1 M in THF) was added and the mixture was stirred at 0-10° C. for 2 hrs. After that time, the pH of the mixture was adjusted to ˜pH 1-2 by the addition of 2 M aqueous HCl and was then extracted with EtOAc (2×600 mL). The pH of the aqueous layer was then adjusted to pH 9-10 by the addition of saturated aqueous sodium carbonate, and then extracted with EtOAc (5×800 mL). The combined organic layers were dried over anhydrous Na₂SO₄, filtered and concentrated. The residue was purified by silica gel chromatography (1-10% MeOH/DCM). The isolated material was dissolved in DMF (250 mL), ACN (75 mL) and MeOH (25 mL) and was allowed to stand at room temperature overnight. The mixture was then poured into water (1 L) and stirred at room temperature for 1 h. The solids were collected by filtration and the filtrate was concentrated to afford a yellow solid. The solids were combined, dissolved in DCM (300 mL) and MeOH (100 mL) and then concentrated to remove the DCM. MeOH (200 mL) was added and the mixture was stirred at 0° C. for 3 hrs. The suspension was filtered and the solids were suspended in MeOH (200 mL). The mixture was heated at 70° C. for 16 hrs, then filtered and the filtrate was concentrated to afford the title compound as a yellow solid (12.0 g, 48%). MS (ESI): Mass calcd. for C₂₁H₁₉DN₈O₃, 433.4; m/z found, 434.2 [M+H]⁺. ¹H NMR (400 MHz, DMSO-d₆) δ 9.90 (s, 1H), 8.47 (d, J=7.3 Hz, 1H), 8.25-8.12 (m, 2H), 7.82 (s, 1H), 7.65-7.56 (m, 1H), 7.17 (s, 1H), 6.89-6.67 (m, 2H), 3.78 (s, 3H), 3.54-3.44 (m, 2H), 2.85 (s, 3H), 2.67-2.58 (m, 1H), 2.33-2.26 (m, 1H).

### Example 210: (R)-3-(3-(6-(2-((1-Ethyl-1H-pyrazol-3-yl)amino)pyrimidin-4-yl-6-d)pyridin-2-yl)isoxazol-5-yl)-3-hydroxy-1-methylpyrrolidin-2-one

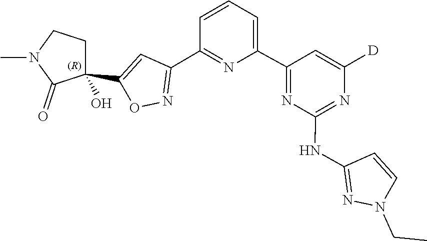

To a solution of (R)-3-(3-(6-(2-chloropyrimidin-4-yl-6-d)pyridin-2-yl)isoxazol-5-yl)-3-hydroxy-1-methylpyrrolidin-2-one (21.5 g, 57.6 mmol, Intermediate 53) in THF (215 mL) was added 1-methyl-1H-pyrazol-3-amine (7.69 g, 69.2 mmol), and the resulting solution was cooled to 0° C. Then, LiHMDS (230 mL, 230 mmol, 1 M in THF) was added and the mixture was stirred at 0° C. for 2 hrs. After that time, the pH of the mixture was adjusted to ˜pH 1-2 by the addition of 2 M aqueous HCl and was then extracted with EtOAc (500 mL). The pH of the aqueous layer was then adjusted to pH 10 by the addition of saturated aqueous sodium carbonate, and then extracted with DCM/MeOH (10/1, 10×500 mL). The combined organic layers were dried over anhydrous Na₂SO₄, filtered and concentrated. The residue was purified by silica gel chromatography (1-6% MeOH/DCM). The isolated material was dissolved in DMF and ACN and was allowed to stand at room temperature for 2 hrs. The mixture was then poured into water (1 L) and stirred at room temperature for 30 min. The solids were collected by filtration and the filtrate was concentrated to afford a yellow solid. The solids were combined, dissolved in DCM/MeOH (3/1, 800 mL) and then concentrated down to 100 mL. MeOH (200 mL) was added and the mixture was stirred at 0° C. for 3 hrs. The suspension was filtered and the filtrate was concentrated to afford the title compound as a yellow solid (12.0 g, 46%). MS (ESI): Mass calcd. for C₂₂H₂₁DN₈O₃, 447.5; m/z found, 448.2 [M+H]⁺. ¹H NMR (400 MHz, DMSO-d₆) δ 9.92 (s, 1H), 8.50-8.43 (m, 1H), 8.25-8.13 (m, 2H), 7.82 (s, 1H), 7.67 (d, J=2.0 Hz, 1H), 7.18 (s, 1H), 6.79 (br s, 1H), 6.73 (d, J=2.0 Hz, 1H), 4.07 (q, J=7.2 Hz, 2H), 3.54-3.42 (m, 2H), 2.86 (s, 3H), 2.67-2.58 (m, 1H), 2.35-2.25 (m, 1H), 1.39 (t, J=7.2 Hz, 3H).

### Example 211: (R)-3-(3-(6-(2-((1-(Difluoromethyl)-3-methyl-1H-pyrazol-4-yl)amino)pyrimidin-4-yl)pyridin-2-yl)isoxazol-5-yl)-3-hydroxy-1-methylpyrrolidin-2-one

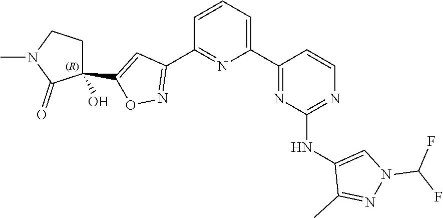

A mixture of (R)-3-hydroxy-1-methyl-3-(3-(6-(2-(methylsulfonyl)pyrimidin-4-yl)pyridin-2-yl)isoxazol-5-yl)pyrrolidin-2-one (150 mg, 0.361 mmol, Intermediate 13), 1-(difluoromethyl)-3-methyl-1H-pyrazol-4-amine hydrochloride (375 mg, 2.04 mmol) and TsOH (75 mg, 0.436 mmol) in 1,4-dioxane (6 mL) was heated at 115° C. for 16 hrs. The mixture was cooled to room temperature, diluted with DCM (30 mL), washed with 2 N aqueous NaOH (30 mL) and brine (30 mL), dried over anhydrous Na₂SO₄, filtered and concentrated. The residue was purified by reverse-phase HPLC (Xtimate C18 column, 5 μm, 30×150 mm; 27-57% ACN/H₂O (with 0.05% NH₃ in water and 10 mM NH₄HCO₃)) to provide the title compound as a white solid (39 mg, 21%). MS (ESI): Mass calcd. for C₂₂H₂₀F₂N₈O₃, 482.5; m/z found, 483.2 [M+H]⁺. ¹H NMR (400 MHz, DMSO-d₆) δ 9.34 (s, 1H), 8.73-8.65 (m, 1H), 8.49-8.40 (m, 2H), 8.27-8.12 (m, 2H), 7.91-7.56 (m, 2H), 7.18 (s, 1H), 6.80 (s, 1H), 3.54-3.45 (m, 2H), 2.85 (s, 3H), 2.66-2.58 (m, 1H), 2.32-2.25 (m, 4H).

### Example 212: (R)-3-(3-(6-(2-((1-Cyclopropyl-1H-pyrazol-3-yl)amino)pyrimidin-4-yl)pyridin-2-yl)isoxazol-5-yl)-3-hydroxy-1-methylpyrrolidin-2-one

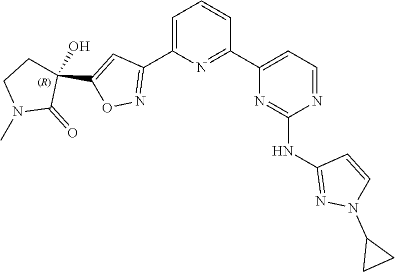

The title compound was prepared using conditions analogous to those described in Example 211 using 1-cyclopropyl-1H-pyrazol-3-amine in place of 1-(difluoromethyl)-3-methyl-1H-pyrazol-4-amine hydrochloride. The reaction mixture was concentrated and purified directly by reverse-phase HPLC (Phenomenex C18 column, 3 μm, 30×75 mm; 35-65% ACN/H₂O (with 0.05% NH₃ in water)) to provide the title compound as a yellow solid (2.7 mg, 4%). MS (ESI): Mass calcd. for C₂₃H₂₂N₈O₃, 458.5; m/z found, 459.2 [M+H]⁺. ¹H NMR (400 MHz, DMSO-d₆) δ 9.91 (s, 1H), 8.64 (d, J=5.2 Hz, 1H), 8.45 (d, J=6.8 Hz, 1H), 8.24-8.11 (m, 2H), 7.82 (d, J=5.2 Hz, 1H), 7.69 (d, J=2.4 Hz, 1H), 7.17 (s, 1H), 6.79-6.68 (m, 2H), 3.65-3.59 (m, 1H), 3.53-3.42 (m, 2H), 2.85 (s, 3H), 2.65-2.59 (m, 1H), 2.32-2.26 (m, 1H), 1.03-0.92 (m, 4H).

### Example 213: (R)-3-Hydroxy-1-methyl-3-(5-(6-(2-((2-methylpyridin-4-yl)amino)pyrimidin-4-yl)pyridin-2-yl)isoxazol-3-yl)pyrrolidin-2-one

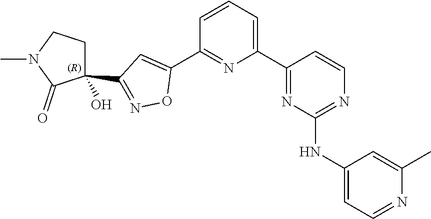

(R)-3-Hydroxy-1-methyl-3-(5-(6-(2-((2-methylpyridin-4-yl)amino)pyrimidin-4-yl)pyridin-2-yl)isoxazol-3-yl)pyrrolidin-2-one was prepared in a manner analogous to Example 109 using N-(2-methylpyridin-4-yl)-4-(tributylstannyl)pyrimidin-2-amine (Intermediate 47) in place of N-(5-chloro-1-methyl-1H-pyrazol-4-yl)-4-(tributylstannyl)pyrimidin-2-amine and (R)-3-(5-(6-bromopyridin-2-yl)isoxazol-3-yl)-3-hydroxy-1-methylpyrrolidin-2-one (Intermediate 52) in place of (R)-3-(1-(6-bromopyridin-2-yl)-1H-1,2,3-triazol-4-yl)-4,4-difluoro-3-hydroxy-1-methylpyrrolidin-2-one (47.9 mg, 17%). MS (ESI): Mass calcd. for C₂₃H₂₁N₇O₃, 443.5; m/z found, 444.2 [M+H]⁺. ¹H NMR (400 MHz, DMSO-d₆) δ 10.20 (s, 1H), 8.83-8.78 (m, 1H), 8.51-8.45 (m, 1H), 8.32-8.24 (m, 2H), 8.17-8.11 (m, 1H), 7.99-7.95 (m, 1H), 7.75-7.65 (m, 2H), 7.36 (s, 1H), 6.57 (s, 1H), 3.52-3.36 (m, 2H), 2.82 (s, 3H), 2.77-2.65 (m, 1H), 2.44 (s, 3H), 2.31-2.21 (m, 1H).

### Example 214: (R)-3-(1-(6-(2-((1-(2,2-Difluoroethyl)-1H-pyrazol-3-yl)amino)pyrimidin-4-yl)pyridin-2-yl)-1H-1,2,3-triazol-4-yl)-3-hydroxy-1-methylpyrrolidin-2-one

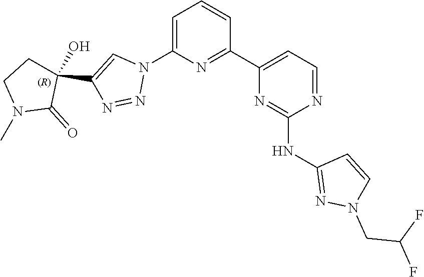

The title compound was prepared using conditions analogous to those described in Example 166 using (R)-3-(1-(6-bromopyridin-2-yl)-1H-1,2,3-triazol-4-yl)-3-hydroxy-1-methylpyrrolidin-2-one (Intermediate 51) in place of 3-(5-(6-chloropyridin-2-yl)-1-methyl-1H-pyrazol-3-yl)-3-hydroxy-1-methylpyrrolidin-2-one and N-(1-(2,2-difluoroethyl)-1H-pyrazol-3-yl)-4-(tributylstannyl)pyrimidin-2-amine (Intermediate 68) in place of N-(1-methyl-1H-pyrazol-3-yl)-4-(tributylstannyl)pyrimidin-2-amine to provide the title compound as a white solid (2.12 mg, 1%). MS (ESI): Mass calcd. for C₂₁H₂₀F₂N₁₀O₂, 482.4; m/z found, 483.2 [M+H]⁺. ¹H NMR (400 MHz, DMSO-d₆) δ 9.68 (br s, 1H), 8.89 (s, 1H), 8.71-8.64 (m, 1H), 8.49-8.43 (m, 1H), 8.33-8.29 (m, 1H), 8.27-8.20 (m, 1H), 7.84-7.78 (m, 1H), 7.75-7.69 (m, 1H), 6.85-6.79 (m, 1H), 6.48-6.16 (m, 1H), 6.09 (br s, 1H), 4.63-4.47 (m, 2H), 3.55-3.46 (m, 2H), 2.90-2.76 (m, 4H), 2.38-2.26 (m, 1H).

### Example 215: (R)-3-(3-(6-(2-((1-(Azetidin-3-yl)-1H-pyrazol-4-yl)amino)pyrimidin-4-yl)pyridin-2-yl)isoxazol-5-yl)-3-hydroxy-1-methylpyrrolidin-2-one

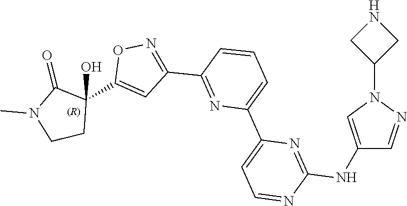

Step A: tert-Butyl (R)-3-(4-((4-(6-(5-(3-hydroxy-1-methyl-2-oxopyrrolidin-3-yl)isoxazol-3-yl)pyridin-2-yl)pyrimidin-2-yl)amino)-1H-pyrazol-1-yl)azetidine-1-carboxylate. tert-Butyl (R)-3-(4-((4-(6-(5-(3-hydroxy-1-methyl-2-oxopyrrolidin-3-yl)isoxazol-3-yl)pyridin-2-yl)pyrimidin-2-yl)amino)-1H-pyrazol-1-yl)azetidine-1-carboxylate was prepared in a manner analogous to Example 86. The title compound was purified using RP-HPLC with a basic modifier (NH₄OH). LCMS (ESI): Mass calcd. for C₂₈H₃₁N₉O₅ 573.6 m/z, found 574.2 [M+H]⁺.

Step B. (R)-3-(3-(6-(2-((1-(Azetidin-3-yl)-1H-pyrazol-4-yl)amino)pyrimidin-4-yl)pyridin-2-yl)isoxazol-5-yl)-3-hydroxy-1-methylpyrrolidin-2-one. tert-Butyl (R)-3-(4-((4-(6-(5-(3-hydroxy-1-methyl-2-oxopyrrolidin-3-yl)isoxazol-3-yl)pyridin-2-yl)pyrimidin-2-yl)amino)-1H-pyrazol-1-yl)azetidine-1-carboxylate was dissolved in DCM (1.4 mL) and 1,4-dioxane (1.4 mL). TFA (3 mL) was added at room temperature. After 12 hours, the reaction mixture was purified via RP HPLC (basic conditions with column C8). ¹H NMR (400 MHz, DMSO-d₆) δ 9.68 (s, 1H), 8.70-8.61 (m, 1H), 8.49 (s, 1H), 8.24-8.11 (m, 3H), 7.81-7.75 (m, 1H), 7.70 (s, 1H), 7.19-7.14 (m, 1H), 6.78 (s, 1H), 5.20 (s, 1H), 3.95-3.83 (m, 2H), 3.78-3.70 (m, 2H), 3.53-3.41 (m, 3H), 2.85 (s, 3H), 2.67-2.59 (m, 2H), 2.34-2.25 (m, 1H).

The compounds shown in Table 3 were prepared in a manner analogous to (R)-3-(3-(6-(2-((1H-pyrazol-3-yl)amino)pyrimidin-4-yl)pyridin-2-yl)isoxazol-5-yl)-3-hydroxy-1-methylpyrrolidin-2-one (Example 31) using the appropriate amine in place of tert-butyl 3-amino-1H-pyrazole-1-carboxylate and the appropriate sulfone in place of (R)-3-hydroxy-1-methyl-3-(3-(6-(2-(methylsulfonyl)pyrimidin-4-yl)pyridin-2-yl)isoxazol-5-yl)pyrrolidin-2-one. Purification methods are noted in the table.

TABLE 3

| | | | Purification | Yield |
|---|---|---|---|---|
| Ex. | Structure Name | Analytical data | conditions | (%) |
| 216 | 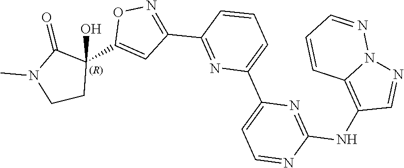 (R)-3-Hydroxy-1-methyl-3-(3- (6-(2-(pyrazolo[1,5-b]pyridazin- 3-ylamino)pyrimidin-4- | ¹H NMR (500 MHz, DMSO-d₆): δ 9.94 (s, 1H), 8.70 (d, J = 5.0 Hz, 1H), 8.57 (s, 1H), 8.52- 8.41 (m, 2H), 8.41- 8.35 (m, 1H), 8.26- 8.12 (m, 2H), 7.87 (d, J = 5.0 Hz, 1H), 7.19 (s, 1H), 7.14-7.01 (m, 1H), 6.78 (s, 1H), 3.56-3.40 (m, 2H), 2.86 (s, 3H), 2.71-2.58 (m, 1H), 2.35-2.25 (m, 1H). MS (ESI⁺): m/z = 477.3. | RP HPLC (TFA modifier) followed by further purification by RP HPLC (NH₄OH modifier) | 13 |
| | yl)pyridin-2-yl)isoxazol-5- | | | |
| | yl)pyrrolidin-2-one | | | |
| 217 | 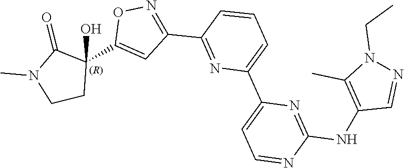 (R)-3-(3-(6-(2-((1-Ethyl-5- methyl-1H-pyrazol-4- | ¹H NMR (500 MHz, DMSO-d₆) δ 8.95-8.82 (m, 1H), 8.66-8.50 (m, 1H), 8.50-8.29 (m, 1H), 8.25-8.08 (m, 2H), 7.74 (d, J = 5.0 Hz, 1H), 7.70- 7.54 (m, 1H), 7.16 (s, 1H), 4.07 (q, J = 7.2 Hz, 2H), 3.59-3.42 (m, 2H), 2.68-2.56 (m, 1H), 2.36- 2.25 (m, 1H), 1.33 (t, J = 7.2 Hz, 3H). MS (ESI⁺): m/z = 461.4. | RP HPLC (NH₄OH modifier) followed by further purification by RP HPLC (TFA modifier) | 6 |
| | yl)amino)pyrimidin-4-yl)pyridin- | | | |
| | 2-yl)isoazol-5-yl)-3-hydroxy-1- | | | |
| | methylpyrrolidin-2-one | | | |
| 218 | 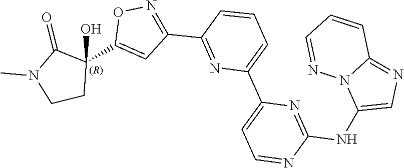 (R)-3-Hydroxy-3-(3-(6-(2- | ¹H NMR (500 MHz, DMSO-d₆) δ 9.56-9.50 (m, 1H), 9.29-9.26 (m, 2H), 9.26-9.22 (m, 1H), 8.81-8.76 (m, 1H), 8.72-8.68 (m, 2H), 8.36-8.29 (m, 3H), 8.12-8.04 (m, 1H), 6.71 (s, 2H), 3.58-3.44 (m, 2H), 2.87 (s, 3H), 2.33 (ddd, J = 13.6, 8.0, 5.4 Hz, 2H). MS (ESI⁺): | RP HPLC (TFA modifier) | 25 |
| | (imidazo[1,2-b]pyridazin-3- | m/z = 469.6. | | |
| | ylamino)pyrimidin-4-yl)pyridin- | | | |
| | 2-yl)isoxazol-5-yl)-1- | | | |
| | methylpyrrolidin-2-one | | | |
| 219 | 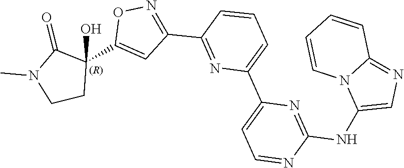 | ¹H NMR (500 MHz, DMSO-d₆) δ 9.30-9.22 (m, 2H), 8.88-8.82 (m, 1H), 8.79-8.74 (m, 1H), 8.65-8.60 (m, 1H), 8.38-8.25 (m, 3H), 8.19 (s, 1H), 7.88-7.79 (m, 1H), 7.27 (s, 1H), 6.87-6.74 (m, 1H), 6.38-6.27 (m, 2H), 3.60-3.43 (m, 2H), | RP HPLC (TFA modifier) | 75 |
| | (R)-3-Hydroxy-3-(3-(6-(2- | 2.87 (s, 3H), 2.69-2.61 | | |
| | (imidazo[1,2-a]pyridin-3- | (m, 1H), 2.39-2.28 (m, | | |
| | ylamino)pyrimidin-4-yl)pyridin- | 1H). MS (ESI⁺): m/z = | | |
| | 2-yl)isoxazol-5-yl)-1- | 469.6. | | |
| | methylpyrrolidin-2-one | | | |
| 220 | 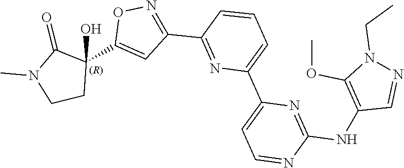 | ¹H NMR (500 MHz, DMSO-d₆) δ 8.60 (s, 1H), 8.56 (d, J = 5.0 Hz, 1H), 8.40 (s, 1H), 8.21-8.09 (m, 2H), 7.87-7.75 (m, 1H), 7.74 (d, J = 5.0 Hz, 1H), 7.16 (s, 1H), 4.00 (q, J = 7.2 Hz, 2H), 3.83 (s, 3H), 3.66-3.35 (m, 2H), 2.86 (s, 3H), 2.68- 2.57 (m, 1H), 2.34- | RP HPLC (NH₄OH modifier) followed by further purification by RP HPLC (TFA modifier) | 16 |
| | (R)-3-(3-(6-(2-((1-Ethyl-5- | 2.25 (m, 1H), 1.36 (t, J = | | |
| | methoxy-1H-pyrazol-4- | 7.2 Hz, 3H). MS (ESI⁺): | | |
| | yl)amino)pyrimidin-4-yl)pyridin- | m/z = 477.3. | | |
| | 2-yl)isoxazol-5-yl)-3-hydroxy-1- | | | |
| | methylpyrrolidin-2-one | | | |
| 221 | 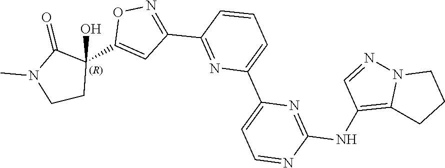 (R)-3-(3-(6-(2-((5,6-Dihydro-4H- pyrrolo[1,2-b]pyrazol-3- yl)amino)pyrimidin-4-yl)pyridin- | ¹H NMR (500 MHz, DMSO-d₆) 8 9.23 (s, 1H), 8.59 (d, J = 5.0 Hz, 1H), 8.45-8.34 (m, 1H), 8.22- 8.10 (m, 2H), 7.73 (d, J = 5.0 Hz, 1H), 7.69 (s, 1H), 7.16 (s, 1H), 6.77 (s, 1H), 4.12-4.01 (m, 2H), 3.53-3.38 (m, 2H), 2.93-2.88 (m, 2H), 2.67- 2.58 (m, 1H), 2.57- 2.51 (m, 2H), 2.35-2.25 (m, 1H). MS (ESI⁺): m/z = 459.6. | RP HPLC (NH₄OH modifier) | 42 |
| | 2-yl)isoxazol-5-yl)-3-hydroxy-1- | | | |
| | methylpyrrolidin-2-one | | | |
| 222 | 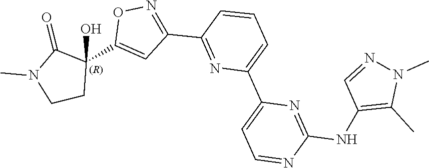 (R)-3-(3-(6-(2-((1,5-Dimethyl- 1H-pyrazol-4- | ¹H NMR (400 MHz, DMSO-d₆) δ 8.89 (s, 1H), 8.56 (d, J = 5.0 Hz, 1H), 8.47-8.31 (m, 1H), 8.23- 8.09 (m, 2H), 7.73 (d, J = 5.0 Hz, 1H), 7.60 (s, 1H), 7.16 (s, 1H), 6.77 (s, 1H), 3.75 (s, 3H), 3.55-3.40 (m, 2H), 2.86 (s, 3H), 2.69-2.57 (m, 1H), 2.35-2.25 (m, 1H), 2.21 (s, 3H). MS (ESI⁺): m/z = 446.3. | RP HPLC (NH₄OH modifier) | 53 |
| | yl)amino)pyrimidin-4-yl)pyridin- | | | |
| | 2-yl)isoxazol-5-yl)-3-hydroxy-1- | | | |
| | methylpyrrolidin-2-one | | | |
| 223 | 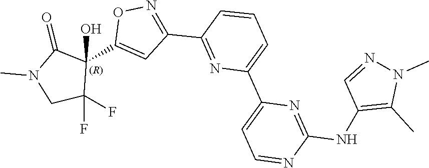 | ¹H NMR (500 MHz, DMSO-d₆) δ 8.89 (s, 1H), 8.56 (d, J = 5.0 Hz, 1H), 8.39 (s, 1H), 8.28-8.11 (m, 2H), 7.97 (s, 1H), 7.75 (d, J = 5.0 Hz, 1H), 7.60 (s, 1H), 7.30 (s, 1H), 4.08-3.93 (m, 2H), 3.75 (s, 3H), 2.97 (s, 3H), 2.21 (s, 3H). MS (ESI⁺): m/z = 482.2. | RP HPLC (NH₄OH modifier) | 55 |
| | (R)-3-(3-(6-(2-((1,5-Dimethyl- | | | |
| | 1H-pyrazol-4- | | | |
| | yl)amino)pyrimidin-4-yl)pyridin- | | | |
| | 2-yl)isoxazol-5-yl)-4,4-difluoro- | | | |
| | 3-hydroxy-1-methylpyrrolidin-2- | | | |
| | one | | | |
| 224 | 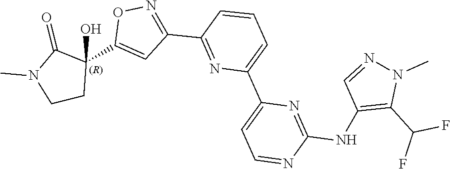 (R)-3-(3-(6-(2-((5- (Difluoromethyl)-1-methyl-1H- pyrazol-4-yl)amino)pyrimidin-4- | ¹H NMR (600 MHz, DMSO-d₆) δ 9.36 (s, 1H), 8.63-8.53 (m, 1H), 8.38- 8.32 (m, 1H), 8.17- 8.04 (m, 2H), 7.88-7.81 (m, 1H), 7.81-7.74 (m, 1H), 7.28 (t, J = 51.7 Hz, 1H), 7.10 (s, 1H), 6.69 (s, 1H), 3.88 (s, 3H), 3.47-3.40 (m, 1H), 3.40- 3.34 (m, 1H), 2.78 (s, 3H), 2.60-2.53 (m, 1H), 2.27-2.19 (m, 1H). MS (ESI⁺): m/z = 482.3. | RP HPLC (NH₄OH modifier) | 7 |
| | yl)pyridin-2-yl)isoxazol-5-yl)-3- | | | |
| | hydroxy-1-methylpyrrolidin-2- | | | |
| | one | | | |
| 225 | 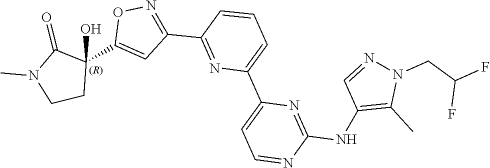 (R)-3-(3-(6-(2-((1-(2,2- Difluoroethyl)-5-methyl-1H- | ¹H NMR (600 MHz, DMSO-d₆) δ 8.88 (s, 1H), 8.57-8.43 (m, 1H), 8.29 (s, 1H), 8.13-8.03 (m, 2H), 7.75-7.57 (m, 2H), 7.09 (s, 1H), 6.41- 6.19 (m, 1H), 4.55- 4.40 (m, 2H), 3.47- 3.40 (m, 1H), 3.39- 3.34 (m, 1H), 2.78 (s, 3H), 2.59-2.50 (m, 1H), 2.27-2.19 (m, 1H). MS (ESI⁺): m/z = 496.3. | RP HPLC (NH₄OH modifier) | 19 |
| | pyrazol-4-yl)amino)pyrimidin-4- | | | |
| | yl)pyridin-2-yl)isoxazol-5-yl)-3- | | | |
| | hydroxy-1-methylpyrrolidin-2- | | | |
| | one | | | |
| 226 | 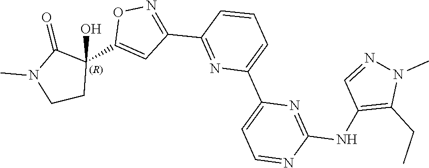 (R)-3-(3-(6-(2-((5-Ethyl-1- methyl-1H-pyrazol-4- yl)amino)pyrimidin-4-yl)pyridin- 2-yl)isoxazol-5-yl)-3-hydroxy-1- methylpyrrolidin-2-one | ¹H NMR (600 MHz, DMSO-d₆) δ 8.84-8.70 (m, 1H), 8.51-8.44 (m, 1H), 8.33-8.24 (m, 1H), 8.13-8.03 (m, 2H), 7.68-7.63 (m, 1H), 7.59-7.46 (m, 1H), 7.08 (s, 1H), 6.85-6.53 (m, 1H), 3.70 (s, 3H), 3.46-3.41 (m, 1H), 3.41-3.35 (m, 1H), 2.78 (s, 3H), 2.65-2.59 (m, 2H), 2.59-2.52 (m, 1H), 2.26-2.20 (m, 1H), 1.03 (t, J = 7.5 Hz, 3H). MS (ESI⁺): m/z = 460.4. | RP HPLC (NH₄OH modifier) | 35 |
| 227 | 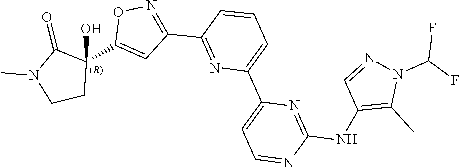 | LCMS (ESI⁺): mass calcd. for C₂₂H₂₀F₂N₈O₃ 482.16 m/z, found 483.15 [M + H]⁺. | RP HPLC (NH₄OH modifier) | 6 |
| | (R)-3-(3-(6-(2-((1- | | | |
| | (Difluoromethyl)-5-methyl-1H- | | | |
| | pyrazol-4-yl)amino)pyrimidin-4- | | | |
| | yl)pyridin-2-yl)isoxazol-5-yl)-3- | | | |
| | hydroxy-1-methylpyrrolidin-2- | | | |
| | one | | | |
| 228 | 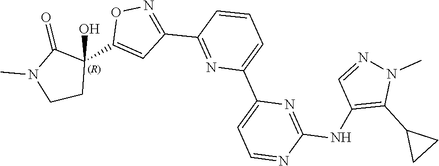 | ¹H NMR (600 MHz, DMSO-d₆) δ 8.61-8.56 (m, 1H), 8.56-8.50 (m, 1H), 8.35 (s, 1H), 8.19- 8.10 (m, 2H), 7.75- 7.67 (m, 1H), 7.45 (s, 1H), 7.15 (s, 1H), 6.76 (s, 1H), 3.84 (s, 3H), 3.55-3.47 (m, 1H), 3.47-3.41 (m, 1H), 2.85 (s, 3H), 2.66-2.56 | RP HPLC (NH₄OH modifier) | 23 |
| | (R)-3-(3-(6-(2-((5-Cyclopropyl- | (m, 1H), 2.34-2.25 (m, | | |
| | 1-methyl-1H-pyrazol-4- | 1H), 1.79-1.68 (m, 1H), | | |
| | yl)amino)pyrimidin-4-yl)pyridin- | 0.87-0.80 (m, 2H), | | |
| | 2-yl)isoxazol-5-yl)-3-hydroxy-1- | 0.72-0.64 (m, 2H). MS | | |
| | methylpyrrolidin-2-one | (ESI⁺): m/z = 472.4. | | |
| 229 | 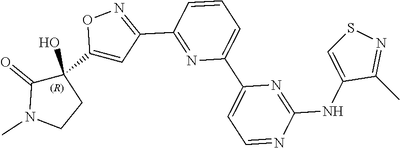 | ¹H NMR (400 MHz, Methanol-d₄) δ 9.09 (d, J = 0.6 Hz, 1H), 8.63 (d, J = 5.2 Hz, 1H), 8.50 (dd, J = 7.7, 1.2 Hz, 1H), 8.16 (dd, J = 7.9, 1.1 Hz, 1H), 8.09 (t, J = 7.8 Hz, 1H), 7.98 (d, J = 5.2 Hz, 1H), 7.19 (s, 1H), 3.64- 3.56 (m, 2H), 2.98 (s, | RP HPLC (NH₄OH modifier) | 14 |
| | (R)-3-Hydroxy-1-methyl-3-(3- | 3H), 2.77 (ddd, J = 13.5, | | |
| | (6-(2-((3-methylisothiazol-4- | 7.0, 5.0 Hz, 1H), 2.51 (s, | | |
| | yl)amino)pyrimidin-4-yl)pyridin- | 3H), 2.42 (ddd, J = 13.5, | | |
| | 2-yl)isoxazol-5-yl)pyrrolidin-2- | 7.9, 6.6 Hz, 1H). MS | | |
| | one | (ESI⁺): m/z = 449.9. | | |
| 230 | 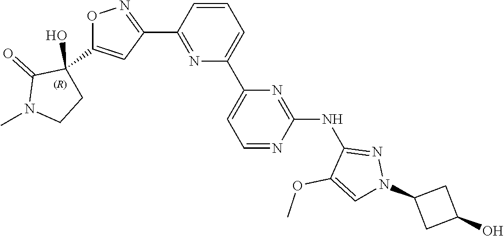 | ¹H NMR (400 MHz, Methanol-d₄) δ 8.57- 8.43 (m, 2H), 8.19 (d, J = 7.7 Hz, 1H), 8.11 (t, J = 7.8 Hz, 1H), 7.92 (d, J = 7.1 Hz, 2H), 7.19 (s, 1H), 4.30-4.22 (m, 1H), 4.14-4.04 (m, 2H), 3.96 (s, 3H), 3.62-3.56 (m, 2H), 2.97 (s, 3H), 2.88-2.75 (m, 3H), 2.51-2.38 (m, 3H). MS (ESI⁺): m/z = 518.7. | RP HPLC (TFA modifier) | 17 |
| | (R)-3-Hydroxy-3-(3-(6-(2-((1- | | | |
| | ((1s,3s)-3-hydroxycyclobutyl)-4- | | | |
| | methoxy-1H-pyrazol-3- | | | |
| | yl)amino)pyrimidin-4-yl)pyridin- | | | |
| | 2-yl)isoxazol-5-yl)-1- | | | |
| | methylpyrrolidin-2-one | | | |
| 231 | 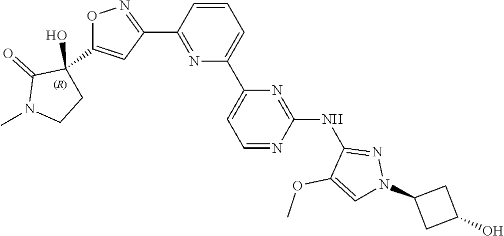 | ¹H NMR (400 MHz, Methanol-d₄) δ 8.55- 8.42 (m, 2H), 8.19 (d, J = 7.6 Hz, 1H), 8.10 (t, J = 7.8 Hz, 1H), 7.94- 7.82 (m, 2H), 7.19 (s, 1H), 4.63-4.54 (m, 1H), 3.96 (s, 3H), 3.63-3.54 (m, 2H), 2.97 (s, 3H), 2.84-2.71 (m, 3H), 2.50-2.38 (m, 3H). MS (ESI⁺): m/z = 518.7. | RP HPLC (TFA modifier) | 9 |
| | (R)-3-Hydroxy-3-(3-(6-(2-((1- | | | |
| | ((1r,3r)-3-hydroxycyclobutyl)-4- | | | |
| | methoxy-1H-pyrazol-3- | | | |
| | yl)amino)pyrimidin-4-yl)pyridin- | | | |
| | 2-yl)isoxazol-5-yl)-1- | | | |
| | methylpyrrolidin-2-one | | | |
| 232 | 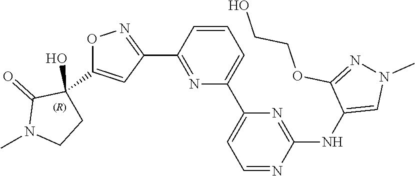 | ¹H NMR (400 MHz, Methanol-d₄) δ 8.57- 8.48 (m, 2H), 8.17- 8.11 (m, 1H), 8.08 (t, J = 7.8 Hz, 1H), 7.90 (s, 1H), 7.81 (d, J = 5.1 Hz, 1H), 7.18 (s, 1H), 4.29- 4.22 (m, 2H), 3.90- 3.83 (m, 2H), 3.77 (s, 3H), 3.61-3.55 (m, 2H), 2.97 (s, 3H), 2.81-2.71 (m, 1H), 2.48-2.37 (m, | RP HPLC (NH₄OH Modifier) | 57 |
| | (R)-3-Hydroxy-3-(3-(6-(2-((3-(2- | 1H). MS (ESI⁺): m/z = | | |
| | hydroxyethoxy)-1-methyl-1H- | 493.6. | | |
| | pyrazol-4-yl)amino)pyrimidin-4- | | | |
| | yl)pyridin-2-yl)isoxazol-5-yl)-1- | | | |
| | methylpyrrolidin-2-one | | | |
| 233 | 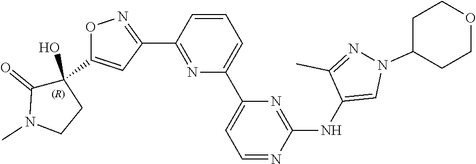 | LCMS (ESI⁺): mass calcd. for C₂₆H₂₈N₈O₄ 516.2 m/z, found 516.7 [M + H]⁺. | RP HPLC (TFA modifier) | 5 |
| | (R)-3-Hydroxy-1-methyl-3-(3- | | | |
| | (6-(2-((3-methyl-1-(tetrahydro- | | | |
| | 2H-pyran-4-yl)-1H-pyrazol-4- | | | |
| | yl)amino)pyrimidin-4-yl)pyridin- | | | |
| | 2-yl)isoxazol-5-yl)pyrrolidin-2- | | | |
| | one | | | |
| 234 | 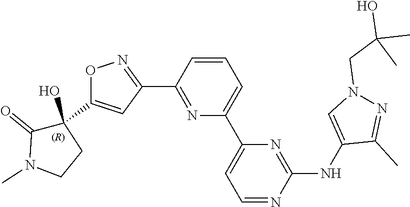 | 1H NMR (400 MHz, Methanol-d₄) δ 8.58- 8.46 (m, 2H), 8.20 (dd, J = 7.9, 1.1 Hz, 1H), 8.10 (t, J = 7.8 Hz, 1H), 8.06- 7.94 (m, 2H), 7.19 (s, 1H), 4.08 (s, 2H), 3.59 (ddt, J = 10.3, 6.9, 3.2 Hz, 2H), 2.97 (s, 3H), 2.77 (ddd, J = 13.6, 7.1, 5.0 Hz, 1H), 2.42 (ddd, J = 13.5, 8.0, 6.5 Hz, 1H), 2.27 (s, 3H), 1.24 (s, 6H). MS (ESI+): m/z = | RP HPLC (TFA modifier) | 34 |
| | (R)-3-Hydroxy-3-(3-(6-(2-((1-(2- | 504.6. | | |
| | hydroxy-2-methylpropyl)-3- | | | |
| | methyl-1H-pyrazol-4- | | | |
| | yl)amino)pyrimidin-4-yl)pyridin- | | | |
| | 2-yl)isoxazol-5-yl)-1- | | | |
| | methylpyrrolidin-2-one | | | |
| 235 | 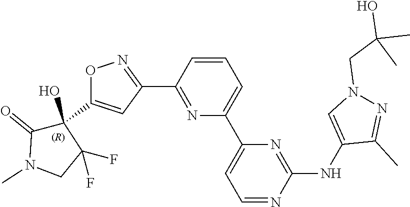 | ¹H NMR (400 MHz, Methanol-d₄) δ 8.56 (d, J = 7.8 Hz, 1H), 8.50 (d, J = 5.7 Hz, 1H), 8.23 (d, J = 7.8 Hz, 1H), 8.12 (t, J = 7.8 Hz, 1H), 8.07- 7.95 (m, 2H), 7.31 (s, 1H), 4.08 (s, 2H), 4.06- 3.94 (m, 2H), 3.06 (s, 3H), 2.27 (s, 3H), 1.24 (s, 6H). MS (ESI+): m/z = 541.3. | RP HPLC (TFA modifier) | 37 |
| | (R)-4,4-Difluoro-3-hydroxy-3-(3- | | | |
| | (6-(2-((1-(2-hydroxy-2- | | | |
| | methylpropyl)-3-methyl-1H- | | | |
| | pyrazol-4-yl)amino)pyrimidin-4- | | | |
| | yl)pyridin-2-yl)isoxazol-5-yl)-1- | | | |
| | methylpyrrolidin-2-one | | | |
| 236 | 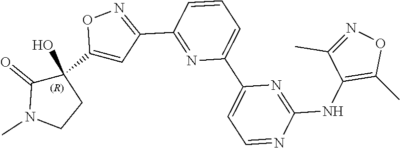 | ¹H NMR (400 MHz, Methanol-d₄) δ 8.51 (d, J = 5.3 Hz, 1H), 8.39 (s, 1H), 8.19-8.12 (m, 1H), 8.05 (t, J = 7.8 Hz, 1H), 7.94 (d, J = 5.3 Hz, 1H), 7.18 (s, 1H), 3.62-3.54 (m, 2H), 2.97 (s, 3H), 2.76 (ddd, J = 13.4, 7.0, 5.0 Hz, 1H), 2.47-2.38 | RP HPLC (TFA modifier) | 25 |
| | (R)-3-(3-(6-(2-((3,5- | (m, 1H), 2.36 (s, 3H), | | |
| | Dimethylisoxazol-4- | 2.19 (s, 3H). MS (ESI⁺): | | |
| | yl)amino)pyrimidin-4-yl)pyridin- | m/z = 447.8. | | |
| | 2-yl)isoxazol-5-yl)-3-hydroxy-1- | | | |
| | methylpyrrolidin-2-one | | | |
| 237 | 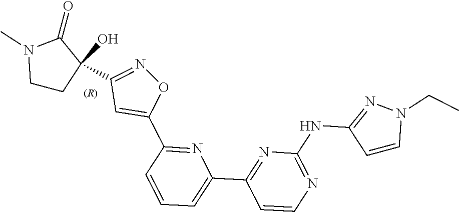 | ¹H NMR (500 MHz, DMSO-d₆) δ 9.91 (s, 1H), 8.65 (d, J = 5.0 Hz, 1H), 8.45 (dd, J = 7.9, 1.0 Hz, 1H), 8.24 (t, J = 7.8 Hz, 1H), 8.11 (dd, J = 7.9, 1.0 Hz, 1H), 7.79 (d, J = 5.0 Hz, 1H), 7.67 (d, J = 2.2 Hz, 1H), 7.35 (s, 1H), 6.72 (d, J = 2.3 Hz, 1H), 6.57 (s, 1H), 4.07 (q, J = 7.3 Hz, 2H), 3.50- 3.46 (m, 1H), 3.46- 3.39 (m, 2H), 2.82 (s, | RP HPLC (NH₄OH Modifier) | 40 |
| | (R)-3-(5-(6-(2-((1-Ethyl-1H- | 3H), 2.75-2.69 (m, 1H), | | |
| | pyrazol-3-yl)amino)pyrimidin-4- | 2.31-2.24 (m, 1H), | | |
| | yl)pyridin-2-yl)isoxazol-3-yl)-3- | 1.39 (t, J = 7.3 Hz, 3H). | | |
| | hydroxy-1-methylpyrrolidin-2- | MS (ESI⁺): m/z = 447.1. | | |
| | one | | | |
| 238 | 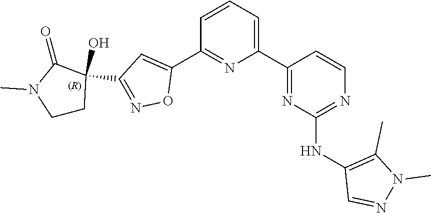 | ¹H NMR (500 MHz, DMSO-d₆) δ 8.94 (s, 1H), 8.57 (d, J = 5.0 Hz, 1H), 8.36 (s, 1H), 8.20 (t, J = 7.8 Hz, 1H), 8.10 (dd, J = 7.8, 1.0 Hz, 1H), 7.71 (d, J = 5.0 Hz, 1H), 7.61 (s, 1H), 7.33 (s, 1H), 3.75 (s, 4H), 3.50-3.44 (m, 1H), 3.44-3.38 (m, 1H), 2.82 (s, 3H), 2.75- 2.69 (m, 1H), 2.30- 2.23 (m, 1H), 2.21 (s, 3H). MS (ESI⁺): m/z = | RP HPLC (TFA modifier) | 63 |
| | (R)-3-(5-(6-(2-((1,5-Dimethyl- | 447.3. | | |
| | 1H-pyrazol-4- | | | |
| | yl)amino)pyrimidin-4-yl)pyridin- | | | |
| | 2-yl)isoxazol-3-yl)-3-hydroxy-1- | | | |
| | methylpyrrolidin-2-one | | | |
| 239 | 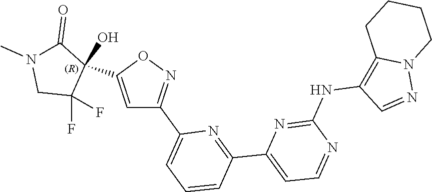 (R)-4,4-Difluoro-3-hydroxy-1- methyl-3-(3-(6-(2-((4,5,6,7- tetrahydropyrazolo[1,5- | ¹H NMR (500 MHz, DMSO-d₆) δ 8.95 (s, 1H), 8.57 (d, J = 5.0 Hz, 1H), 8.40 (s, 1H), 8.22-8.19 (m, 1H), 8.17 (dd, J = 7.8, 1.6 Hz, 1H), 7.96 (s, 1H), 7.75 (d, J = 5.0 Hz, 1H), 7.67 (s, 1H), 7.30 (s, 1H), 4.08-4.05 (m, 2H), 4.05-3.99 (m, 2H), 2.98 (s, 3H), 2.71 (t, J = 6.4 Hz, 2H), 2.00-1.95 (m, 2H), 1.82-1.77 (m, 2H). MS (ESI⁺): m/z = 509.3. | RP HPLC (TFA modifier) | 8 |
| | a]pyridin-3-yl)amino)pyrimidin- | | | |
| | 4-yl)pyridin-2-yl)isoxazol-5- | | | |
| | yl)pyrrolidin-2-one | | | |
| 240 | 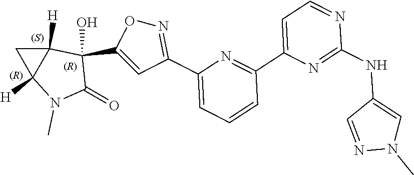 (1R,4R,5S)-4-Hydroxy-2- methyl-4-(3-(6-(2-((1-methyl- 1H-pyrazol-4- yl)amino)pyrimidin-4-yl)pyridin- | ¹H NMR (500 MHz, DMSO-d₆) δ 9.65 (s, 1H), 8.64 (d, J = 5.0 Hz, 1H), 8.53-8.48 (m, 1H), 8.24-8.19 (m, 1H), 8.19-8.16 (m, 1H), 7.98 (s, 1H), 7.78 (d, J = 5.0 Hz, 1H), 7.60 (s, 1H), 7.19 (s, 1H), 3.86 (s, 3H), 3.48-3.43 (m, 1H), 2.86 (s, 3H), 2.13- 2.08 (m, 1H), 1.00- 0.94 (m, 1H), 0.85- 0.81 (m, 1H). MS (ESI⁺): m/z = 445.0. | RP HPLC (TFA modifier) | 70 |
| | 2-yl)isoxazol-5-yl)-2- | | | |
| | azabicyclo[3.1.0]hexan-3-one | | | |
| 241 | 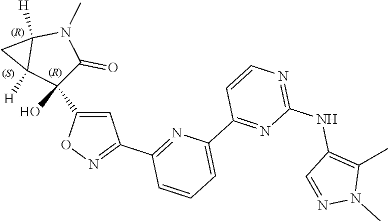 | ¹H NMR (500 MHz, DMSO-d₆) δ 8.93 (s, 1H), 8.56 (d, J = 5.0 Hz, 1H), 8.38 (s, 1H), 8.20-8.16 (m, 1H), 8.16-8.13 (m, 1H), 7.75 (d, J = 5.0 Hz, 1H), 7.61 (s, 1H), 7.17 (s, 1H), 3.75 (s, 4H), 3.47-3.43 (m, 1H), 2.86 (s, 3H), 2.22 (s, 3H), 2.13-2.07 (m, 1H), 0.99-0.94 (m, 1H), 0.85-0.81 (m, 1H). MS (ESI⁺): m/z = 459.3. | RP HPLC (TFA modifier) | 61 |
| | (1R,4R,5S)-4-(3-(6-(2-((1,5- | | | |
| | Dimethyl-1H-pyrazol-4- | | | |
| | yl)amino)pyrimidin-4-yl)pyridin- | | | |
| | 2-yl)isoxazol-5-yl)-4-hydroxy-2- | | | |
| | methyl-2- | | | |
| | azabicyclo[3.1.0]hexan-3-one | | | |
| 242 | 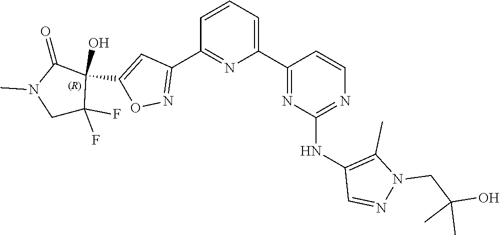 | ¹H NMR (500 MHz, DMSO-d₆) δ 8.88 (s, 1H), 8.57 (d, J = 5.0 Hz, 1H), 8.39 (s, 1H), 8.20-8.15 (m, 2H), 7.96 (s, 1H), 7.75 (d, J = 5.0 Hz, 1H), 7.66 (s, 1H), 7.30 (s, 1H), 4.69 (s, 1H), 4.03 (t, J = 12.9, 11.6 Hz, 2H), 3.97 (s, 2H), 2.97 (s, 3H), 2.24 (s, 3H), 1.14 (s, 6H). MS (ESI⁺): m/z = 541.3. | RP HPLC (NH₄OH Modifier) | 52 |
| | (R)-4,4-Difluoro-3-hydroxy-3-(3- | | | |
| | (6-(2-((1-(2-hydroxy-2- | | | |
| | methylpropyl)-5-methyl-1H- | | | |
| | pyrazol-4-yl)amino)pyrimidin-4- | | | |
| | yl)pyridin-2-yl)isoxazol-5-yl)-1- | | | |
| | methylpyrrolidin-2-one | | | |
| 243 | 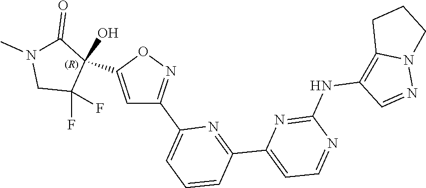 (R)-3-(3-(6-(2-((5,6-Dihydro-4H- pyrrolo[1,2-b]pyrazol-3- yl)amino)pyrimidin-4-yl)pyridin- | ¹H NMR (500 MHz, DMSO-d₆) δ 9.23 (s, 1H), 8.58 (d, J = 5.0 Hz, 1H), 8.42 (d, J = 7.5 Hz, 1H), 8.21 (t, J = 7.6 Hz, 1H), 8.18 (dd, J = 7.8, 1.5 Hz, 1H), 7.97 (s, 1H), 7.74 (d, J = 5.0 Hz, 1H), 7.68 (s, 1H), 7.30 (s, 1H), 4.07 (t, J = 7.3 Hz, 2H), 4.03 (t, J = 12.8, 11.7 Hz, 2H), 2.98 (s, 3H), 2.90 (t, J = 7.3 Hz, 2H), 2.57-2.53 (m, 2H). MS (ESI⁺): m/z = 495.1. | RP HPLC (NH₄OH Modifier) | 49 |
| | 2-yl)isoxazol-5-yl)-4,4-difluoro- | | | |
| | 3-hydroxy-1-methylpyrrolidin-2- | | | |
| | one | | | |
| 244 | 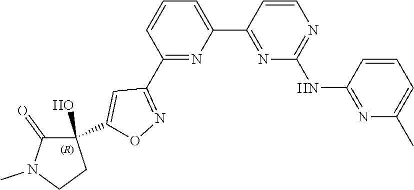 (R)-3-Hydroxy-1-methyl-3-(3- (6-(2-((6-methylpyridin-2- yl)amino)pyrimidin-4-yl)pyridin- 2-yl)isoxazol-5-yl)pyrrolidin-2- one | ¹H NMR (401 MHz, DMSO-d₆) δ 11.13 (s, 1H), 8.90 (d, J = 5.2 Hz, 1H), 8.51 (dd, J = 7.5, 1.4 Hz, 1H), 8.25 (t, J = 7.7 Hz, 1H), 8.21 (dd, J = 7.8, 1.4 Hz, 1H), 8.15 (d, J = 5.2 Hz, 1H), 8.02 (t, J = 8.0 Hz, 1H), 7.91 (d, J = 8.5 Hz, 1H), 7.21 (s, 1H), 7.14 (d, J = 7.4 Hz, 1H), 5.40 (s, 1H), 3.55-3.42 (m, 2H), 2.86 (s, 3H), 2.67-2.61 (m, 1H), 2.59 (s, 3H), 2.36-2.27 (m, 1H). MS (ESI⁺): m/z = 444.2. | RP HPLC (TFA modifier) | 19 |
| 245 | 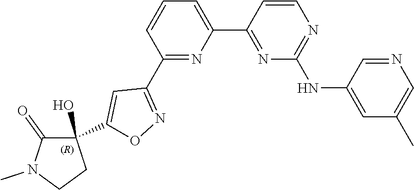 (R)-3-Hydroxy-1-methyl-3-(3- (6-(2-((5-methylpyridin-3- | ¹H NMR (401 MHz, DMSO-d₆) δ 9.36 (t, J = 2.1 Hz, 1H), 9.33 (d, J = 5.1 Hz, 1H), 9.26 (s, 1H), 8.91-8.85 (m, 2H), 8.34-8.27 (m, 2H), 7.77-7.72 (m, 1H), 7.29 (s, 1H), 6.93 (s, 1H), 3.91 (s, 1H), 3.56- 3.42 (m, 3H), 2.87 (s, 3H), 2.68-2.59 (m, 1H), 2.51 (p, J = 1.7 Hz, 3H), 2.37-2.28 (m, 1H). MS (ESI⁺): m/z = 444.2. | RP HPLC (TFA modifier) | 21 |
| | yl)amino)pyrimidin-4-yl)pyridin- | | | |
| | 2-yl)isoxazol-5-yl)pyrrolidin-2- | | | |
| | one | | | |
| 246 | 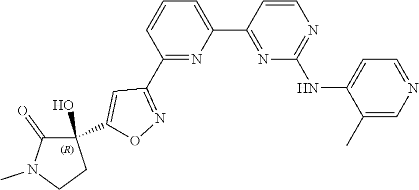 (R)-3-Hydroxy-1-methyl-3-(3- (6-(2-((3-methylpyridin-4- yl)amino)pyrimidin-4-yl)pyridin- 2-yl)isoxazol-5-yl)pyrrolidin-2- | ¹H NMR (500 MHz, DMSO-d₆) δ 9.43 (dd, J = 7.6, 2.0 Hz, 1H), 9.38 (s, 1H), 9.20 (d, J = 5.1 Hz, 1H), 9.15 (s, 1H), 8.92 (dd, J = 7.0, 1.9 Hz, 1H), 8.68 (d, J = 5.1 Hz, 1H), 8.48 (s, 1H), 8.33- 8.27 (m, 2H), 7.27 (s, 1H), 7.08 (d, J = 7.6 Hz, 1H), 3.55-3.49 (m, 1H), 3.49-3.43 (m, 1H), 2.87 (s, 3H), 2.67-2.60 (m, 1H), 2.35-2.29 (m, 4H). MS (ESI⁺): m/z = 444.2. | RP HPLC (TFA modifier) | 14 |
| | one | | | |
| 247 | 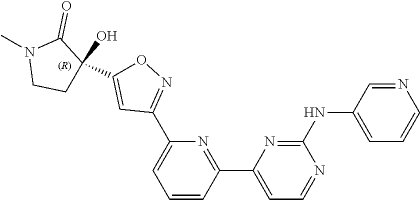 (R)-3-Hydroxy-1-methyl-3-(3- | ¹H NMR (401 MHz, DMSO-d₆) δ 9.50 (t, J = 2.1 Hz, 1H), 9.41 (dt, J = 5.9, 1.4 Hz, 1H), 9.34 (d, J = 5.1 Hz, 1H), 8.90- 8.84 (m, 2H), 8.35- 8.28 (m, 2H), 8.01- 7.91 (m, 2H), 7.30 (s, 1H), 7.02 (s, 1H), 6.83 (s, 1H), 3.56-3.48 (m, 2H), 2.87 (s, 3H), 2.68- 2.58 (m, 1H), 2.37- 2.28 (m, 1H). MS (ESI⁺): m/z = 430.3. | RP HPLC (TFA modifier) | 7 |
| | (6-(2-(pyridin-3- | | | |
| | ylamino)pyrimidin-4-yl)pyridin- | | | |
| | 2-yl)isoxazol-5-yl)pyrrolidin-2- | | | |
| | one | | | |
| 248 | 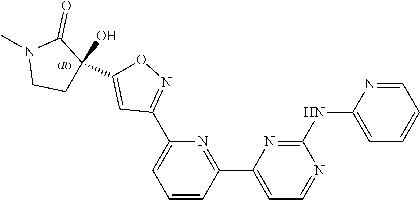 | ¹H NMR (401 MHz, DMSO-d₆) δ 9.29 (d, J = 5.2 Hz, 2H), 8.83 (d, J = 5.2 Hz, 1H), 8.66-8.56 (m, 2H), 8.31-8.20 (m, 2H), 8.10-8.01 (m, 1H), 7.31-7.26 (m, 2H), 7.12-7.04 (m, 1H), 4.20 (s, 1H), 3.57-3.41 (m, 2H), 2.87 (s, 3H), 2.68-2.58 (m, 1H), 2.38-2.27 (m, 1H). MS (ESI⁺): m/z = 430.5. | RP HPLC (TFA modifier) | 6 |
| | (R)-3-Hydroxy-1-methyl-3-(3- | | | |
| | (6-(2-(pyridin-2- | | | |
| | ylamino)pyrimidin-4-yl)pyridin- | | | |
| | 2-yl)isoxazol-5-yl)pyrrolidin-2- | | | |
| | one | | | |
| 249 | 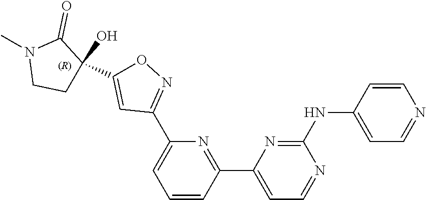 | ¹H NMR (500 MHz, DMSO-d₆) δ 9.48-9.44 (m, 2H), 9.21 (d, J = 5.1 Hz, 1H), 8.98 (s, 2H), 8.86 (dd, J = 6.7, 2.1 Hz, 1H), 8.68 (d, J = 5.1 Hz, 1H), 8.32-8.27 (m, 2H), 7.27 (s, 1H), 7.09-7.06 (m, 2H), 3.55-3.49 (m, 1H), 3.49-3.44 (m, 1H), 2.87 (s, 3H), 2.67-2.60 (m, 2H), 2.36-2.29 (m, 1H). MS (ESI⁺): m/z = | RP HPLC (TFA modifier) | 9 |
| | (R)-3-Hydroxy-1-methyl-3-(3- | 430.2. | | |
| | (6-(2-(pyridin-4- | | | |
| | ylamino)pyrimidin-4-yl)pyridin- | | | |
| | 2-yl)isoxazol-5-yl)pyrrolidin-2- | | | |
| | one | | | |
| 250 | 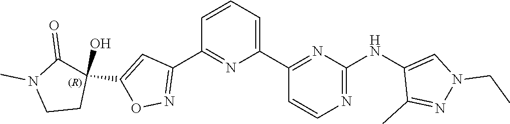 | ¹H NMR (500 MHz, DMSO-d₆) δ 8.92 (s, 1H), 8.59 (d, J = 5.0 Hz, 1H), 8.46-8.37 (m, 1H), 8.21-8.12 (m, 2H), 7.94 (s, 1H), 7.74 (d, J = 5.0 Hz, 1H), 7.16 (s, 1H), 6.76 (s, 1H), 4.07 (q, J = 7.3 Hz, 2H), 3.53- | RP HPLC (NH₄OH Modifier) | 15 |
| | (R)-3-(3-(6-(2-((1-Ethyl-3- | 3.42 (m, 2H), 2.85 (s, | | |
| | methyl-1H-pyrazol-4- | 3H), 2.65-2.59 (m, 2H), | | |
| | yl)amino)pyrimidin-4-yl)pyridin- | 2.33-2.26 (m, 1H), | | |
| | 2-yl)isoxazol-5-yl)-3-hydroxy-1- | 2.15 (s, 3H), 1.37 (t, J = | | |
| | methylpyrrolidin-2-one | 7.3 Hz, 3H). MS (ESI⁺): | | |
| | | m/z = 461.2. | | |
| 251 | 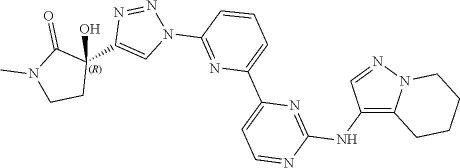 (R)-3-Hydroxy-1-methyl-3-(1- (6-(2-((4,5,6,7- tetrahydropyrazolo[1,5- | ¹H NMR (500 MHz, CD₃OD) δ 8.94 (s, 1H), 8.52-8.44 (m, 2H), 8.32-8.27 (m, 1H), 8.25-8.19 (m, 1H), 7.87-7.83 (m, 1H), 7.73 (s, 1H), 4.19-4.12 (m, 2H), 3.66-3.55 (m, 2H), 2.99-2.89 (m, 5H), 2.80-2.73 (m, 2H), 2.47-2.39 (m, 1H), 2.15-2.07 (m, 2H), 1.95-1.87 (m, 2H) .. MS (ESI⁺): m/z = 473.2. | RP HPLC (TFA modifier) | 10 |
| | a]pyridin-3-yl)amino)pyrimidin- | | | |
| | 4-yl)pyridin-2-yl)-1H-1,2,3- | | | |
| | triazol-4-yl)pyrrolidin-2-one | | | |
| 252 | 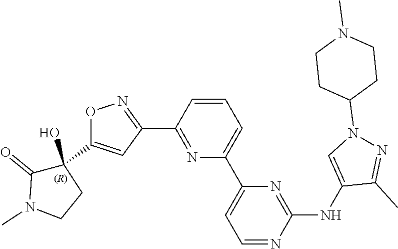 | ¹H NMR (400 MHz, CD₃OD) δ 9.02-8.96 (m, 1H), 8.68-8.63 (m, 1H), 8.38-8.33 (m, 1H), 8.28-8.21 (m, 2H), 7.71-7.67 (m, 1H), 7.67-7.59 (m, 1H), 7.22 (s, 1H), 6.78 (s, 1H), 6.71-6.67 (m, 1H), 3.53-3.42 (m, 2H), 3.04-2.99 (m, 3H), 2.86 (s, 3H), 2.66-2.59 (m, 1H), 2.37-2.27 (m, 1H).MS (ESI⁺): m/z = 530.2. | RP HPLC (TFA modifier) | 41 |
| | (R)-3-Hydroxy-3-(3-(6-(2-((5- | | | |
| | (hydroxymethyl)pyrazin-2- | | | |
| | yl)amino)pyrimidin-4-yl)pyridin- | | | |
| | 2-yl)isoxazol-5-yl)-1- | | | |
| | methylpyrrolidin-2-one | | | |
| 253 | 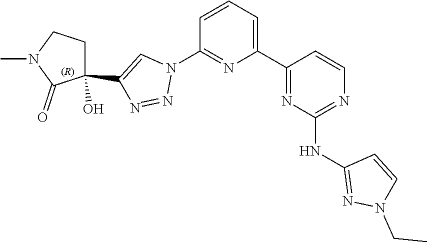 (3R)-3-[1-[6-[2-[(1-Ethylpyrazol- 3-yl)amino]pyrimidin-4-yl]-2- | ¹H NMR (400 MHz, DMSO-d₆) δ 9.98 (s, 1H), 8.95 (s, 1H), 8.69- 8.65 (m, 1H), 8.48-8.44 (m, 1H), 8.38-8.32 (m, 1H), 8.28-8.24 (m, 1H), 7.84-7.81 (m, 1H), 7.69- 7.66 (m, 1H), 6.75- 6.72 (m, 1H), 6.36 (s, 1H), 4.13-4.01 (m, 2H), 3.53-3.46 (m, 2H), 2.87- 2.71 (m, 4H), 2.35- 2.23 (m, 1H), 1.43-1.34 (m, 3H). MS (ESI): Mass calcd. for C₂₁H₂₂N₁₀O₂, 446.5; m/z found, 447.2 [M + H]⁺. | RP HPLC (TFA modifier) | 7 |
| | pyridyl]triazol-4-yl]-3-hydroxy-1- | | | |
| | methyl-pyrrolidin-2-one | | | |

The compounds shown in Table 4 were prepared in a manner analogous to (R)-3-(3-(6-(2-((5-fluoropyridin-3-yl)amino)pyrimidin-4-yl)pyridin-2-yl)isoxazol-5-yl)-3-hydroxy-1-methylpyrrolidin-2-one (Example 86) using the appropriate amine in place of 3-fluoro-5-aminopyridine and appropriate chloride in place of (3r)-3-[3-[6-(2-chloropyrimidin-4-yl)-2-pyridyl]isoxazol-5-yl]-3-hydroxy-1-methyl-pyrrolidin-2-one. Purification methods are noted in the table.

TABLE 4

| | | | Purification | Yield |
|---|---|---|---|---|
| Ex. | Structure Name | Analytical data | conditions | (%) |
| 254 | 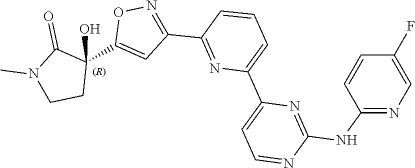 (R)-3-(3-(6-(2-((5-Fluoropyridin- 2-yl)amino)pyrimidin-4- yl)pyridin-2-yl)isoxazol-5-yl)-3- | ¹H NMR (500 MHz, DMSO-d₆) δ 10.26 (s, 1H), 8.78 (d, J = 5.1 Hz, 1H), 8.59-8.46 (m, 1H), 8.43-8.32 (m, 2H), 8.25-8.14 (m, 2H), 8.05-7.97 (m, 1H), 7.91-7.74 (m, 1H), 7.20 (s, 1H), 3.58-3.36 (m, 2H), 2.86 (s, 3H), 2.68- 2.57 (m, 1H), 2.36- 2.23 (m, 1H). MS (ESI⁺): m/z = 447.2. | RP HPLC (TFA modifier) | 33 |
| | hydroxy-1-methylpyrrolidin-2- | | | |
| | one | | | |
| 255 | 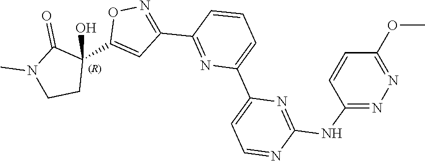 (R)-3-Hydroxy-3-(3-(6-(2-((6- methoxypyridazin-3- yl)amino)pyrimidin-4-yl)pyridin- 2-yl)isoxazol-5-yl)-1- | ¹H NMR (500 MHz, DMSO-d₆) δ 10.86 (s, 1H), 8.80 (d, J = 5.1 Hz, 1H), 8.55-8.44 (m, 1H), 8.38 (d, J = 9.6 Hz, 1H), 8.27- 8.14 (m, 2H), 8.06 (d, J = 5.0 Hz, 1H), 7.42 (d, J = 9.5 Hz, 1H), 7.20 (s, 1H), 4.03 (s, 3H), 2.86 (s, 3H), 2.70- 2.55 (m, 1H), 2.38- 2.22 (m, 1H). MS (ESI⁺): m/z = 460.3. | RP HPLC (TFA modifier) | 10 |
| | methylpyrrolidin-2-one | | | |
| 256 | 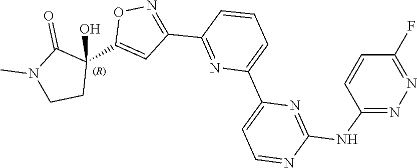 | LCMS (ESI⁺): mass calcd. for C₂₂H₂₀N₈O₃ 444.2 m/z, found 444.3 [M + H]⁺. | RP HPLC (TFA modifier) | 5 |
| | (R)-3-Hydroxy-1-methyl-3-(3- | | | |
| | (6-(2-((6-methylpyridazin-3- | | | |
| | yl)amino)pyrimidin-4-yl)pyridin- | | | |
| | 2-yl)isoxazol-5-yl)pyrrolidin-2- | | | |
| | one | | | |
| 257 | 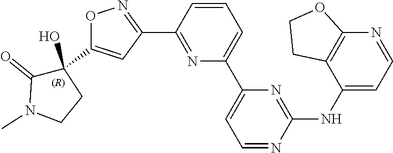 (R)-3-(3-(6-(2-((2,3- Dihydrofuro[2,3-b]pyridin-4- yl)amino)pyrimidin-4-yl)pyridin- 2-yl)isoxazol-5-yl)-3-hydroxy-1- methylpyrrolidin-2-one | ¹H NMR (401 MHz, DMSO-d₆) δ 10.17 (s, 1H), 8.85 (d, J = 5.1 Hz, 1H), 8.48 (dd, J = 7.1, 1.8 Hz, 1H), 8.27- 8.16 (m, 2H), 8.11 (d, J = 5.1 Hz, 1H), 7.99 (q, J = 6.5 Hz, 2H), 7.20 (s, 1H), 4.75 (t, J = 8.7 Hz, 2H), 3.57-3.41 (m, 2H), 3.38 (t, J = 8.7 Hz, 2H), 2.86 (s, 3H), 2.69-2.59 (m, 1H), 2.38-2.28 (m, 1H). MS (ESI⁺): m/z = 472.1. | RP HPLC (TFA modifier) | 13 |
| 258 | 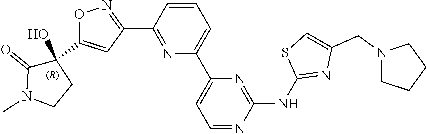 | ¹H NMR (401 MHz, DMSO-d₆) δ 11.98 (s, 1H), 8.87 (d, J = 5.1 Hz, 1H), 8.69 (d, J = 7.7 Hz, 1H), 8.32- 8.19 (m, 2H), 8.09 (d, J = 5.1 Hz, 1H), 7.21 (s, 1H), 7.19 (s, 1H), 6.79 (s, 1H), 4.06 (s, 2H), | RP HPLC (NH₄OH Modifier) | 11 |
| | (R)-3-Hydroxy-1-methyl-3-(3- | 3.58-3.44 (m, 2H), | | |
| | (6-(2-((4-(pyrrolidin-1- | 3.00 (s, 4H), 2.86 (s, | | |
| | ylmethyl)thiazol-2- | 3H), 2.63 (ddd, J = | | |
| | yl)amino)pyrimidin-4-yl)pyridin- | 13.0, 7.6, 5.0 Hz, 1H), | | |
| | 2-yl)isoxazol-5-yl)pyrrolidin-2- | 2.31 (ddd, J = 13.6, | | |
| | one | 8.0, 5.6 Hz, 1H), 1.91- | | |
| | | 1.78 (m, 4H). MS | | |
| | | (ESI⁺): m/z = 519.3. | | |
| 259 | 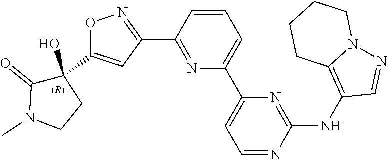 | ¹H NMR (400 MHz, Methanol-d₄) δ 8.47 (d, J = 5.1 Hz, 1H), 8.13 (dd, J = 7.8, 1.1 Hz, 1H), 8.05 (t, J = 7.8 Hz, 1H), 7.82 (d, J = 5.2 Hz, 1H), 7.65 (s, 1H), 7.18 (s, 1H), 4.13 (t, J = 6.1 Hz, 2H), 3.62- 3.56 (m, 2H), 2.97 (s, 3H), 2.82-2.70 (m, | RP HPLC (NH₄OH Modifier) | 35 |
| | (R)-3-Hydroxy-1-methyl-3-(3- | 3H), 2.46-2.37 (m, | | |
| | (6-(2-((4,5,6,7- | 1H), 2.12-2.04 (m, | | |
| | tetrahydropyrazolo[1,5- | 2H), 1.95-1.84 (m, | | |
| | a]pyridin-3-yl)amino)pyrimidin- | 2H). MS (ESI⁺): m/z = | | |
| | 4-yl)pyridin-2-yl)isoxazol-5- | 472.6. | | |
| | yl)pyrrolidin-2-one | | | |
| 260 | 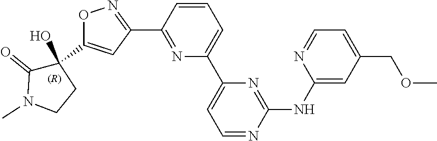 | ¹H NMR (401 MHz, DMSO-d₆) δ 8.89 (d, J = 5.2 Hz, 1H), 8.53 (dd, J = 7.2, 1.7 Hz, 1H), 8.38 (d, J = 5.9 Hz, 1H), 8.27-8.17 (m, 4H), 7.99 (s, 1H), 7.31- 7.04 (m, 3H), 3.58- 3.47 (m, 2H), 2.64 (ddd, J = 13.0, 7.7, 5.1 | RP HPLC (TFA modifier) | 26 |
| | (R)-3-Hydroxy-3-(3-(6-(2-((4- | Hz, 1H), 2.47 (s, 3H), | | |
| | (methoxymethyl)pyridin-2- | 2.36-2.30 (m, 1H). | | |
| | yl)amino)pyrimidin-4-yl)pyridin- | MS (ESI⁺): m/z = | | |
| | 2-yl)isoxazol-5-yl)-1- | 474.7. | | |
| | methylpyrrolidin-2-one | | | |
| 261 | 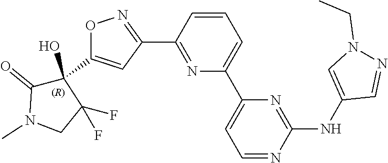 (R)-3-(3-(6-(2-((1-Ethyl-1H- pyrazol-4-yl)amino)pyrimidin-4- yl)pyridin-2-yl)isoxazol-5-yl)- 4,4-difluoro-3-hydroxy-1- | ¹H NMR (400 MHz, Methanol-d₄) δ 8.59- 8.51 (m, 2H), 8.19 (dd, J = 7.9, 1.1 Hz, 1H), 8.11 (t, J = 7.8 Hz, 1H), 8.04 (d, J = 0.8 Hz, 1H), 7.86 (d, J = 5.2 Hz, 1H), 7.68 (s, 1H), 7.30 (s, 1H), 4.21 (q, J = 7.3 Hz, 2H), 4.08- 3.92 (m, 2H), 3.06 (s, 3H), 1.49 (t, J = 7.3 Hz, 3H). MS (ESI⁺): m/z = 505. | RP HPLC (TFA modifier) | 12 |
| | methylpyrrolidin-2-one | | | |
| 262 | 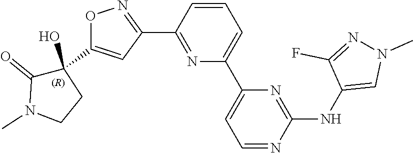 | ¹H NMR (400 MHz, Methanol-d₄) δ 8.53 (d, J = 5.1 Hz, 1H), 8.48 (d, J = 7.8 Hz, 1H), 8.14 (dd, J = 7.8, 1.2 Hz, 1H), 8.06 (t, J = 7.8 Hz, 1H), 7.88-7.84 (m, 2H), 7.18 (s, 1H), 3.79 (d, J = 1.1 Hz, 3H), 3.62-3.54 (m, | RP HPLC (NH₄OH Modifier) | 26 |
| | (R)-3-(3-(6-(2-((3-Fluoro-1- | 2H), 2.97 (s, 3H), 2.76 | | |
| | methyl-1H-pyrazol-4- | (ddd, J = 13.5, 6.9, 5.0 | | |
| | yl)amino)pyrimidin-4-yl)pyridin- | Hz, 1H), 2.42 (ddd, J = | | |
| | 2-yl)isoxazol-5-yl)-3-hydroxy-1- | 13.5, 7.9, 6.6 Hz, 1H). | | |
| | methylpyrrolidin-2-one | MS (ESI⁺): m/z = | | |
| | | 450.6. | | |
| 263 | 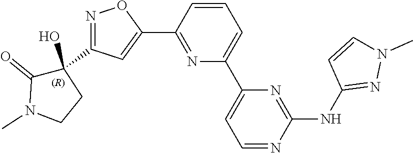 (R)-3-Hydroxy-1-methyl-3-(5- (6-(2-((1-methyl-1H-pyrazol-3- yl)amino)pyrimidin-4-yl)pyridin- 2-yl)isoxazol-3-yl)pyrrolidin-2- one | ¹H NMR (500 MHz, Methanol-d₄) δ 8.61 (dd, J = 5.1, 0.8 Hz, 1H), 8.58-8.51 (m, 1H), 8.13 (t, J = 7.9 Hz, 1H), 8.05 (dd, J = 7.9, 1.1 Hz, 1H), 7.90 (dd, J = 5.1, 1.0 Hz, 1H), 7.53 (d, J = 2.3 Hz, 1H), 7.33 (s, 1H), 6.76 (d, J = 2.3 Hz, 1H), 3.85 (s, 3H), 3.60-3.54 (m, 2H), 2.96 (s, 3H), 2.93- 2.86 (m, 1H), 2.41 (dt, J = 13.3, 7.9 Hz, | RP HPLC (NH₄OH modifier) followed by recrystalli- zation with 95% MeOH/ 5% DCM | 34 |
| | | 1H). MS (ESI⁺): m/z = | | |
| | | 433.2. | | |
| 264 | 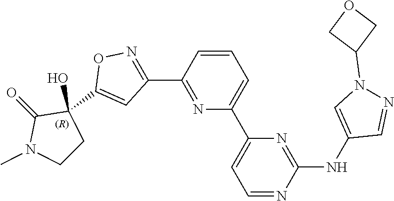 | ¹H NMR (400 MHz, CD₃OD) δ 8.53 (d, J = 5.1 Hz, 1H), 8.51- 8.42 (m, 1H), 8.18 (d, J = 0.8 Hz, 1H), 8.15- 7.99 (m, 2H), 7.80- 7.73 (m, 2H), 7.16 (s, 1H), 5.60-5.50 (m, 1H), 5.13-5.00 (m, 4H), 3.65-3.54 (m, 2H), 2.98 (s, 3H), 2.82- 2.70 (m, 1H), 2.48- 2.36 (m, 1H). MS | RP HPLC (NH₄OH Modifier) | 11 |
| | (R)-3-Hydroxy-1-methyl-3-(3- | (ESI⁺): m/z = 475.7. | | |
| | (6-(2-((1-(oxetan-3-yl)-1H- | | | |
| | pyrazol-4-yl)amino)pyrimidin-4- | | | |
| | yl)pyridin-2-yl)isoxazol-5- | | | |
| | yl)pyrrolidin-2-one | | | |
| 265 | 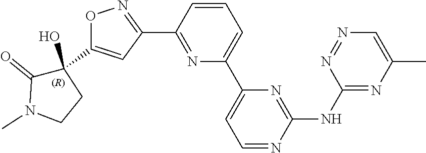 (R)-3-Hydroxy-1-methyl-3-(3- | ¹H NMR (401 MHz, DMSO-d₆) δ 8.99 (s, 1H), 8.84 (d, J = 5.0 Hz, 1H), 8.51 (dd, J = 7.3, 1.6 Hz, 1H), 8.24- 8.10 (m, 5H), 7.21 (s, 1H), 2.84-2.85 (m, 7H), 2.69-2.55 (m, 3H), 2.36-2.25 (m, 3H). MS (ESI⁺): m/z = 446.5. | RP HPLC (NH₄OH Modifier) | 31 |
| | (6-(2-((5-methyl-1,2,4-triazin-3- | | | |
| | yl)amino)pyrimidin-4-yl)pyridin- | | | |
| | 2-yl)isoxazol-5-yl)pyrrolidin-2- | | | |
| | one | | | |
| 266 | 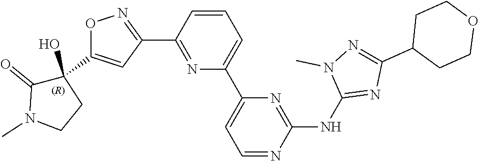 | ¹H NMR (401 MHz, DMSO-d₆) δ 8.71 (d, J = 5.1 Hz, 1H), 8.32 (dd, J = 6.8, 2.1 Hz, 1H), 8.21-8.14 (m, 2H), 8.00 (d, J = 5.1 Hz, 1H), 7.18 (s, 1H), 3.95- 3.87 (m, 4H), 3.50- 3.41 (m, 4H), 2.99- 2.86 (m, 2H), 2.86 (s, 3H), 2.62 (ddd, J = | RP HPLC (TFA modifier) | 11 |
| | (R)-3-Hydroxy-1-methyl-3-(3- | 12.9, 7.6, 5.0 Hz, 1H), | | |
| | (6-(2-((1-methyl-3-(tetrahydro- | 2.30 (ddd, J = 13.5, | | |
| | 2H-pyran-4-yl)-1H-1,2,4-triazol- | 7.9, 5.6 Hz, 1H), 1.94- | | |
| | 5-yl)amino)pyrimidin-4- | 1.81 (m, 2H), 1.80- | | |
| | yl)pyridin-2-yl)isoxazol-5- | 1.64 (m, 2H). MS | | |
| | yl)pyrrolidin-2-one | (ESI⁺): m/z = 518.4. | | |
| 267 | 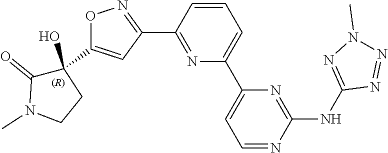 (R)-3-Hydroxy-1-methyl-3-(3- (6-(2-((2-methyl-2H-tetrazol-5- yl)amino)pyrimidin-4-yl)pyridin- | ¹H NMR (401 MHz, DMSO-d₆) δ 8.73 (d, J = 5.1 Hz, 1H), 8.50 (dd, J = 7.1, 1.8 Hz, 1H), 8.25-8.14 (m, 2H), 7.99 (d, J = 5.1 Hz, 1H), 7.19 (s, 1H), 4.36 (s, 3H), 3.55-3.46 (m, 2H), 2.86 (s, 3H), 2.63 (ddd, J = 12.9, 7.6, 5.0 Hz, 1H), 2.30 (ddd, J = 13.5, 7.9, 5.6 Hz, 1H). MS (ESI⁺): m/z = 435.4. | RP HPLC (TFA modifier) | 9 |
| | 2-yl)isoxazol-5-yl)pyrrolidin-2- | | | |
| | one | | | |
| 268 | 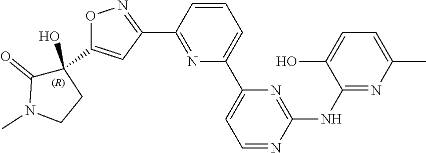 (R)-3-Hydroxy-3-(3-(6-(2-((3- hydroxy-6-methylpyridin-2- yl)amino)pyrimidin-4-yl)pyridin- 2-yl)isoxazol-5-yl)-1- | ¹H NMR (401 MHz, DMSO-d₆) δ 8.82 (d, J = 5.0 Hz, 1H), 8.40- 8.32 (m, 1H), 8.29 (d, J = 5.0 Hz, 1H), 8.19 (d, J = 4.5 Hz, 2H), 7.48 (d, J = 7.8 Hz, 1H), 7.21 (s, 1H), 6.77 (d, J = 10.2 Hz, 1H), 6.57 (d, J = 7.9 Hz, 1H), 2.85 (s, 3H), 2.62 (ddd, J = 13.0, 7.6, 5.1 Hz, 1H), 2.41-2.24 (m, 4H). MS (ESI⁺): m/z = 460.5. | RP HPLC (NH₄OH Modifier) | 16 |
| | methylpyrrolidin-2-one | | | |
| 269 | 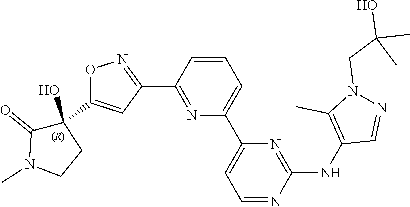 (R)-3-Hydroxy-3-(3-(6-(2-((1-(2- hydroxy-2-methylpropyl)-5- | ¹H NMR (401 MHz, DMSO-d₆) δ 8.97 (s, 1H), 8.57 (d, J = 5.1 Hz, 1H), 8.37 (s, 1H), 8.21-8.11 (m, 2H), 7.75 (d, J = 5.0 Hz, 1H), 7.69 (s, 1H), 7.17 (s, 1H), 3.98 (s, 2H), 3.56-3.40 (m, 2H), 2.86 (s, 3H), 2.63 (ddd, J = 12.9, 7.6, 5.0 Hz, 1H), 2.37-2.28 (m, 1H), 2.25 (s, 3H), 1.15 (s, 6H). MS (ESI⁺): m/z = 505.6. | RP HPLC (TFA modifier) | 17 |
| | methyl-1H-pyrazol-4- | | | |
| | yl)amino)pyrimidin-4-yl)pyridin- | | | |
| | 2-yl)isoxazol-5-yl)-1- | | | |
| | methylpyrrolidin-2-one | | | |
| 270 | 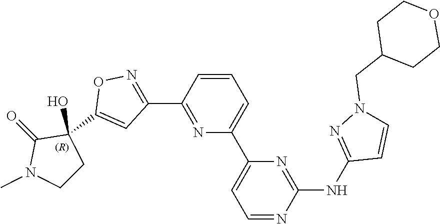 (R)-3-Hydroxy-1-methyl-3-(3- (6-(2-((1-((tetrahydro-2H-pyran- 4-yl)methyl)-1H-pyrazol-3- yl)amino)pyrimidin-4-yl)pyridin- 2-yl)isoxazol-5-yl)pyrrolidin-2-one | ¹H NMR (400 MHz, CD₃OD) δ 8.57 (d, J = 5.1 Hz, 1H), 8.54- 8.44 (m, 1H), 8.16- 8.10 (m, 1H), 8.05 (t, J = 7.8 Hz, 1H), 7.87 (d, J = 5.1 Hz, 1H), 7.55 (d, J = 2.3 Hz, 1H), 7.18 (s, 1H), 6.75 (d, J = 2.3 Hz, 1H), 3.99- 3.92 (m, 4H), 3.62- 3.49 (m, 2H), 3.46- 3.35 (m, 2H), 2.98 (s, 3H), 2.83-2.72 (m, 1H), 2.51-2.37 (m, 1H), 2.20-2.12 (m, 1H), 1.55-1.46 (m, 2H), 1.43-1.33 (m, 2H). MS (ESI⁺): m/z = 517.6. | RP HPLC (NH₄OH Modifier) | 12 |
| 271 | 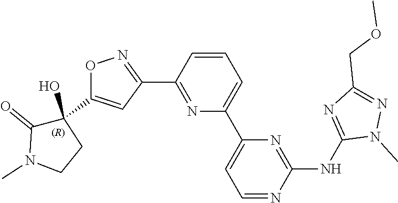 (R)-3-Hydroxy-3-(3-(6-(2-((3- | ¹H NMR (401 MHz, DMSO-d₆) δ 8.72 (d, J = 5.1 Hz, 1H), 8.36- 8.28 (m, 1H), 8.21- 8.15 (m, 2H), 8.01 (d, J = 5.1 Hz, 1H), 7.19 (s, 1H), 4.38 (s, 2H), 3.61- 3.41 (m, 2H), 3.34 (s, 3H), 2.87 (s, 3H), 2.64 (ddd, J = 12.9, 7.6, 5.0 Hz, 1H), 2.32 (ddd, J = 13.5, 7.9, 5.6 Hz, 1H). MS (ESI₊): m/z = 478.6. | RP HPLC (TFA modifier) | 49 |
| | (methoxymethyl)-1-methyl-1H- | | | |
| | 1,2,4-triazol-5- | | | |
| | yl)amino)pyrimidin-4-yl)pyridin- | | | |
| | 2-yl)isoxazol-5-yl)-1- | | | |
| | methylpyrrolidin-2-one | | | |
| 272 | 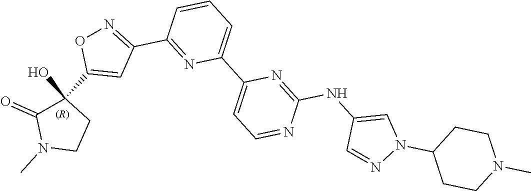 (R)-3-Hydroxy-1-methyl-3-(3- (6-(2-((1-(1-methylpiperidin-4- yl)-1H-pyrazol-4- yl)amino)pyrimidin-4-yl)pyridin- 2-yl)isoxazol-5-yl)pyrrolidin-2-one | ¹H NMR (401 MHz, DMSO-d₆) δ 9.60 (s, 1H), 8.67 (d, J = 5.1 Hz, 1H), 8.37-8.32 (m, 1H), 8.24-8.15 (m, 2H), 7.91 (d, J = 5.0 Hz, 1H), 7.50 (d, J = 1.9 Hz, 1H), 7.17 (s, 1H), 6.78 (s, 1H), 6.56 (s, 1H), 6.35 (d, J = 1.9 Hz, 1H), 4.44-4.32 (m, 1H), 3.52-3.42 (m, 4H), 2.85 (s, 3H), 2.69-2.59 (m, 3H), 2.54 (d, J = 5.3 Hz, 4H), 2.36-2.24 (m, 1H), 2.24-2.13 (m, 2H), 1.97 (d, J = 12.7 | RP HPLC (NH₄OH Modifier) | 2 |
| | | Hz, 2H). MS (ESI⁺): | | |
| | | m/z = 516.3. | | |
| 273 | 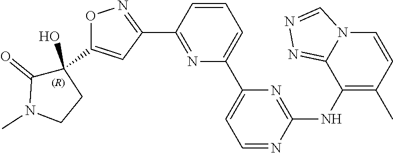 (R)-3-Hydroxy-1-methyl-3-(3- (6-(2-((7-methyl- [1,2,4]triazolo[4,3-a]pyridin-8- yl)amino)pyrimidin-4-yl)pyridin- 2-yl)isoxazol-5-yl)pyrrolidin-2-one | 1H NMR (401 MHz, DMSO-d₆) δ 9.55 (s, 1H), 9.30 (s, 1H), 8.59 (d, J = 5.0 Hz, 1H), 8.50 (d, J = 6.9 Hz, 1H), 8.16-8.07 (m, 3H), 7.85 (d, J = 5.0 Hz, 1H), 7.17 (s, 1H), 7.07 (d, J = 7.0 Hz, 1H), 3.56-3.39 (m, 2H), 2.86 (s, 3H), 2.63 (ddd, J = 12.9, 7.6, 5.0 Hz, 1H), 2.35-2.24 (m, 4H). MS (ESI⁺): m/z = 484.4. | RP HPLC (NH₄OH Modifier) | 13 |
| 274 | 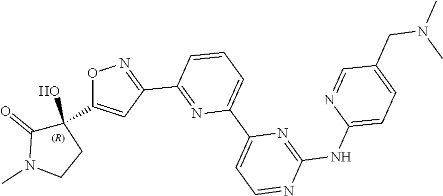 | ¹H NMR (401 MHz, DMSO-d₆) δ 10.07 (s, 1H), 8.78 (d, J = 5.1 Hz, 1H), 8.53 (dd, J = 7.4, 1.5 Hz, 1H), 8.39 (d, J = 8.6 Hz, 1H), 8.28-8.16 (m, 3H), 7.99 (d, J = 5.0 Hz, 1H), 7.81 (dd, J = 8.7, 2.3 Hz, 1H), 7.20 (s, 1H), 6.79 (s, 1H), 3.60 (s, 2H), 3.54-3.43 (m, 3H), 2.86 (s, 3H), 2.63 | RP HPLC (NH₄OH Modifier) | 15 |
| | (R)-3-(3-(6-(2-((5- | (ddd, J = 13.0, 7.6, 5.0 | | |
| | ((Dimethylamino)methyl)pyridin- | Hz, 1H), 2.36-2.27 | | |
| | 2-yl)amino)pyrimidin-4- | (m, 7H). MS (ESI⁺): | | |
| | yl)pyridin-2-yl)isoxazol-5-yl)-3- | m/z = 487.3. | | |
| | hydroxy-1-methylpyrrolidin-2- | | | |
| | one | | | |
| 275 | 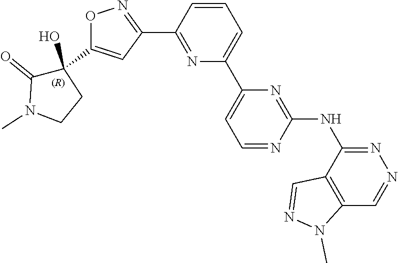 | ¹H NMR (401 MHz, DMSO-d₆) δ 9.42 (s, 1H), 8.89 (d, J = 5.1 Hz, 1H), 8.44 (s, 1H), 8.34 (dd, J = 6.2, 2.7 Hz, 1H), 8.21-8.14 (m, 3H), 7.23 (s, 1H), 6.79 (s, 1H), 4.23 (s, 3H), 3.56-3.42 (m, 2H), 2.86 (s, 3H), 2.70- 2.57 (m, 1H), 2.35- 2.28 (m, 1H). MS (ESI⁺): m/z = 485.3. | RP HPLC (NH₄OH Modifier) | 5 |
| | (R)-3-Hydroxy-1-methyl-3-(3- | | | |
| | (6-(2-((1-methyl-1H- | | | |
| | pyrazolo[3,4-d]pyridazin-4- | | | |
| | yl)amino)pyrimidin-4-yl)pyridin- | | | |
| | 2-yl)isoxazol-5-yl)pyrrolidin-2- | | | |
| | one | | | |
| 276 | 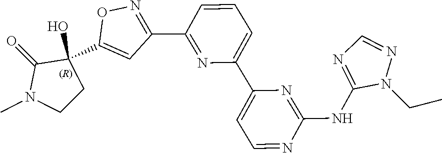 | ¹H NMR (401 MHz, DMSO-d₆) δ 9.94 (s, 1H), 8.68 (d, J = 5.1 Hz, 1H), 8.27 (dd, J = 6.7, 2.3 Hz, 1H), 8.22- 8.12 (m, 2H), 7.97 (d, J = 5.1 Hz, 1H), 7.92 (s, 1H), 7.17 (s, 1H), 6.77 (s, 1H), 4.03 (q, J = 7.2 Hz, 2H), 3.55-3.42 | RP HPLC (NH₄OH Modifier) | 2 |
| | (R)-3-(3-(6-(2-((1-Ethyl-1H- | (m, 2H), 2.85 (s, 3H), | | |
| | 1,2,4-triazol-5- | 2.72-2.59 (m, 1H), | | |
| | yl)amino)pyrimidin-4-yl)pyridin- | 2.35-2.24 (m, 1H), | | |
| | 2-yl)isoxazol-5-yl)-3-hydroxy-1- | 1.33 (t, J = 7.2 Hz, 3H). | | |
| | methylpyrrolidin-2-one | MS (ESI⁺): m/z = | | |
| | | 448.2. | | |
| 277 | 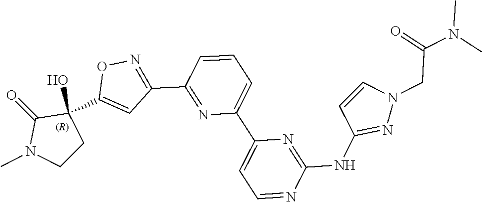 | ¹H NMR (401 MHz, DMSO-d₆) δ 9.95 (s, 1H), 8.66 (d, J = 5.0 Hz, 1H), 8.49 (dd, J = 7.6, 1.3 Hz, 1H), 8.27- 8.12 (m, 2H), 7.83 (d, J = 5.0 Hz, 1H), 7.61 (d, J = 2.3 Hz, 1H), 7.18 (s, 1H), 6.78 (d, J = 2.2 Hz, 2H), 5.01 (s, 2H), 3.56-3.39 (m, 2H), 3.04 (s, 3H), 2.86 (d, J = 1.7 Hz, 6H), 2.63 | RP HPLC (NH₄OH Modifier) | 22 |
| | (R)-2-(3-((4-(6-(5-(3-Hydroxy-1- | (ddd, J = 12.9, 7.6, 5.0 | | |
| | methyl-2-oxopyrrolidin-3- | Hz, 1H), 2.30 (ddd, J = | | |
| | yl)isoxazol-3-yl)pyridin-2- | 13.5, 8.0, 5.7 Hz, 1H). | | |
| | yl)pyrimidin-2-yl)amino)-1H- | MS (ESI⁺): m/z = | | |
| | pyrazol-1-yl)-N,N- | 504.3. | | |
| | dimethylacetamide | | | |
| 278 | 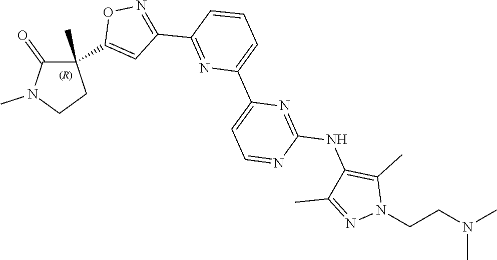 | ¹H NMR (401 MHz, DMSO-d₆) δ 9.63 (s, 1H), 8.72 (s, 1H), 8.61- 8.48 (m, 2H), 8.19- 8.10 (m, 2H), 7.73 (d, J = 5.0 Hz, 1H), 7.15 (s, 1H), 4.38 (t, J = 6.6 Hz, 2H), 3.55 (t, J = 6.6 Hz, 2H), 3.51-3.39 (m, 2H), 2.87 (s, 6H), 2.85 (s, 3H), 2.67-2.56 (m, 1H), 2.33-2.25 (m, 1H), 2.16 (s, 3H), 2.05 (s, 3H). MS (ESI⁺): m/z = 518.3. | RP HPLC (NH₄OH Modifier) | 3 |
| | (R)-3-(3-(6-(2-((1-(2- | | | |
| | (Dimethylamino)ethyl)-3,5- | | | |
| | dimethyl-1H-pyrazol-4- | | | |
| | yl)amino)pyrimidin-4-yl)pyridin- | | | |
| | 2-yl)isoxazol-5-yl)-3-hydroxy-1- | | | |
| | methylpyrrolidin-2-one | | | |
| 279 | 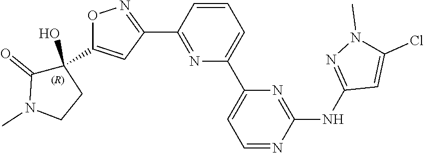 (R)-3-(3-(6-(2-((5-Chloro-1- methyl-1H-pyrazol-3- yl)amino)pyrimidin-4-yl)pyridin- | ¹H NMR (400 MHz, Methanol-d₄) δ 8.63 (d, J = 5.1 Hz, 1H), 8.54 (dd, J = 7.6, 1.3 Hz, 1H), 8.22-8.09 (m, 2H), 7.95 (d, J = 5.1 Hz, 1H), 7.21 (s, 1H), 6.83 (s, 1H), 3.80 (s, 3H), 3.65-3.56 (m, 2H), 3.00 (s, 3H), 2.85- 2.75 (m, 1H), 2.50- 2.38 (m, 1H). MS (ESI⁺): m/z = 467.1. | RP HPLC (TFA modifier) | 26 |
| | 2-yl)isoxazol-5-yl)-3-hydroxy-1- | | | |
| | methylpyrrolidin-2-one | | | |
| 280 | 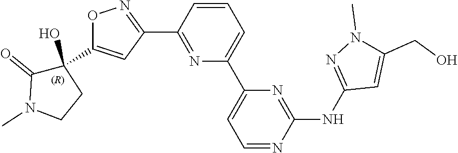 (R)-3-Hydroxy-3-(3-(6-(2-((5- (hydroxymethyl)-1-methyl-1H- pyrazol-3-yl)amino)pyrimidin-4- yl)pyridin-2-yl)isoxazol-5-yl)-1- methylpyrrolidin-2-one | ¹H NMR (400 MHz, Methanol-d₄) δ 8.66 (d, J = 5.8 Hz, 1H), 8.52 (dd, J = 7.7, 1.1 Hz, 1H), 8.19 (dd, J = 7.9, 1.1 Hz, 1H), 8.12 (d, J = 7.8 Hz, 1H), 8.09- 8.04 (m, 1H), 7.18 (s, 1H), 6.41 (s, 1H), 4.65 (s, 2H), 3.88 (s, 3H), 3.64-3.57 (m, 2H), 2.98 (s, 3H), 2.82- 2.72 (m, 1H), 2.48- 2.35 (m, 1H). MS (ESI⁺): m/z = 463.6. | RP HPLC (TFA modifier) | 10 |
| 281 | 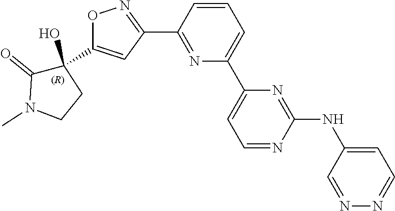 (R)-3-Hydroxy-1-methyl-3-(3- (6-(2-(pyridazin-4- ylamino)pyrimidin-4-yl)pyridin- 2-yl)isoxazol-5-yl)pyrrolidin-2-one | ¹H NMR (400 MHz, Methanol-d₄) δ 9.97 (dd, J = 7.9, 0.6 Hz, 1H), 9.14 (d, J = 5.1 Hz, 1H), 8.79-8.72 (m, 2H), 8.71 (dd, J = 3.1, 0.6 Hz, 1H), 8.26 (dd, J = 7.9, 1.1 Hz, 1H), 8.18 (t, J = 7.8 Hz, 1H), 7.31 (dd, J = 7.8, 3.1 Hz, 1H), 7.24 (s, 1H), 3.66-3.55 (m, 2H), 2.98 (s, 3H), 2.78 (ddd, J = 13.5, 7.1, 5.0 Hz, 1H), 2.43 (ddd, J = 13.5, 8.0, 6.4 Hz, 1H). MS (ESI⁺): m/z = 431.5. | RP HPLC (TFA modifier) | 21 |
| 282 | 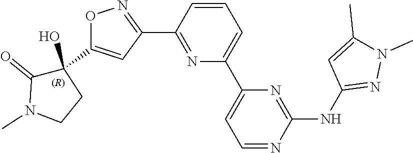 (R)-3-(3-(6-(2-((1,5-Dimethyl- 1H-pyrazol-3- yl)amino)pyrimidin-4-yl)pyridin- 2-yl)isoxazol-5-yl)-3-hydroxy-1- | ¹H NMR (500 MHz, Methanol-d₄) δ 8.59 (d, J = 5.1 Hz, 1H), 8.56 (dd, J = 7.7, 1.1 Hz, 1H), 8.17 (dd, J = 7.8, 1.1 Hz, 1H), 8.11 (t, J = 7.8 Hz, 1H), 7.92 (d, J = 5.1 Hz, 1H), 7.21 (s, 1H), 6.56 (s, 1H), 3.73 (s, 3H), 3.63-3.59 (m, 2H), 3.00 (s, 3H), 2.84- 2.74 (m, 1H), 2.48- 2.42 (m, 1H), 2.35 (s, 3H). MS (ESI⁺): m/z = 447.2. | RP HPLC (NH₄OH Modifier) | 19 |
| | methylpyrrolidin-2-one | | | |
| 283 | 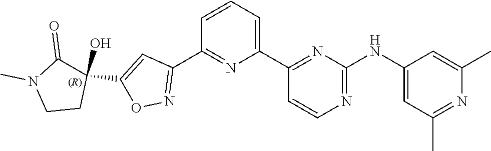 | ¹H NMR (500 MHz, DMSO-d₆) δ 10.10 (s, 1H), 8.80 (d, J = 5.1 Hz, 1H), 8.49 (dd, J = 7.8, 1.1 Hz, 1H), 8.26 (t, J = 7.8 Hz, 1H), 8.19 (dd, J = 7.8, 1.1 Hz, 1H), 7.99 (d, J = 5.0 Hz, 1H), 7.55 (s, 2H), 7.20 (s, 1H), 6.77 (s, | RP HPLC (NH₄OH Modifier) | 9 |
| | (R)-3-(3-(6-(2-((2,6- | 1H), 3.53-3.48 (m, | | |
| | Dimethylpyridin-4- | 1H), 3.48-3.42 (m, | | |
| | yl)amino)pyrimidin-4-yl)pyridin- | 1H), 2.85 (s, 3H), 2.66- | | |
| | 2-yl)isoxazol-5-yl)-3-hydroxy-1- | 2.60 (m, 1H), 2.40 (s, | | |
| | methylpyrrolidin-2-one | 6H), 2.34-2.27 (m, | | |
| | | 1H). MS (ESI⁺): m/z = | | |
| | | 458.3. | | |
| 284 | 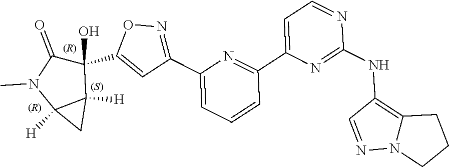 | ¹H NMR (500 MHz, DMSO-d₆) δ 9.23 (s, 1H), 8.58 (d, J = 5.0 Hz, 1H), 8.39 (s, 1H), 8.19 (t, J = 7.7 Hz, 1H), 8.15 (d, J = 6.4 Hz, 1H), 7.73 (d, J = 5.0 Hz, 1H), 7.17 (s, 1H), 6.67 (s, 1H), 4.07 (t, J = 7.1 Hz, 3H), 3.45 (s, 2H), 2.90 (t, J = 7.2 Hz, | RP HPLC (NH₄OH Modifier) | 4 |
| | (1R,4R,5S)-4-(3-(6-(2-((5,6- | 3H), 2.85 (s, 2H), 2.12- | | |
| | Dihydro-4H-pyrrolo[1,2- | 2.06 (m, 2H), 2.02 (d, | | |
| | b]pyrazol-3-yl)amino)pyrimidin- | J = 7.4 Hz, 2H), 1.24 | | |
| | 4-yl)pyridin-2-yl)isoxazol-5-yl)- | (s, 3H), 0.96 (d, J = 8.4 | | |
| | 4-hydroxy-2-methyl-2- | Hz, 1H), 0.83 (s, 2H). | | |
| | azabicyclo[3.1.0]hexan-3-one | MS (ESI⁺): m/z = 471.2. | | |
| 285 | 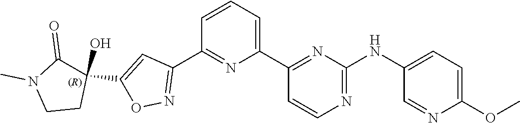 (R)-3-Hydroxy-3-(3-(6-(2-((6- methoxypyridin-3- yl)amino)pyrimidin-4-yl)pyridin- 2-yl)isoxazol-5-yl)-1- methylpyrrolidin-2-one | ¹H NMR (500 MHz, DMSO-d₆) δ 9.73 (s, 1H), 8.68 (d, J = 5.0 Hz, 1H), 8.59 (d, J = 2.7 Hz, 1H), 8.44 (dd, J = 7.7, 1.2 Hz, 1H), 8.22 (t, J = 7.7 Hz, 1H), 8.17 (dd, J = 7.8, 1.2 Hz, 1H), 8.12 (dd, J = 8.9, 2.8 Hz, 1H), 7.87 (d, J = 5.0 Hz, 1H), 7.18 (s, 1H), 6.88-6.85 (m, 1H), 6.77 (s, 1H), 3.86 (s, 3H), 3.54-3.48 (m, | RP HPLC (NH₄OH Modifier) | 26 |
| | | 1H), 3.48-3.43 (m, | | |
| | | 1H), 2.86 (s, 3H), 2.67- | | |
| | | 2.60 (m, 1H), 2.34- | | |
| | | 2.27 (m, 1H). MS | | |
| | | (ESI⁺): m/z = 460.2. | | |
| 286 | 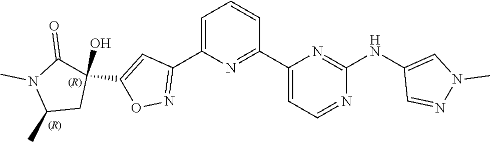 (3R,5R)-3-Hydroxy-1,5- dimethyl-3-(3-(6-(2-((1-methyl- 1H-pyrazol-4- yl)amino)pyrimidin-4-yl)pyridin- 2-yl)isoxazol-5-yl)pyrrolidin-2-one | ¹H NMR (500 MHz, DMSO-d₆) δ 9.64 (s, 1H), 8.64 (d, J = 5.0 Hz, 1H), 8.50 (d, J = 7.8 Hz, 1H), 8.22 (t, J = 7.8 Hz, 1H), 8.17 (dd, J = 7.8, 1.2 Hz, 1H), 7.98 (s, 1H), 7.77 (d, J = 5.0 Hz, 1H), 7.60 (s, 1H), 7.16 (s, 1H), 6.77 (s, 1H), 3.86 (s, 3H), 3.72 (q, J = 6.5 Hz, 1H), 2.85 (dd, J = 13.4, 6.9 Hz, 1H), 2.82 (s, 3H), 1.92 (dd, J = 13.4, 6.3 | RP HPLC (NH₄OH Modifier) | 8 |
| | | Hz, 1H), 1.32 (d, J = | | |
| | | 6.2 Hz, 3H). MS (ESI⁺): | | |
| | | m/z = 447.4. | | |
| 287 | 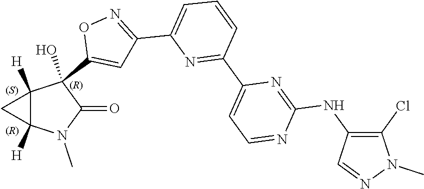 (1R,4R,5S)-4-(3-(6-(2-((5- Chloro-1-methyl-1H-pyrazol-4- yl)amino)pyrimidin-4-yl)pyridin- | ¹H NMR (500 MHz, DMSO-d₆) δ 9.05 (s, 1H), 8.60 (d, J = 5.0 Hz, 1H), 8.38 (d, J = 7.4 Hz, 1H), 8.21- 8.13 (m, 2H), 7.81 (d, J = 5.0 Hz, 1H), 7.79 (s, 1H), 7.18 (s, 1H), 6.68 (s, 1H), 3.84 (s, 3H), 3.47-3.43 (m, 1H), 2.86 (s, 3H), 2.12- 2.07 (m, 1H), 0.99- 0.94 (m, 1H), 0.85- 0.81 (m, 1H). MS (ESI⁺): m/z = 479.1. | FCC (0- 100% EtOAc in Hexanes) | 13 |
| | 2-yl)isoxazol-5-yl)-4-hydroxy-2- | | | |
| | methyl-2- | | | |
| | azabicyclo[3.1.0]hexan-3-one | | | |
| 288 | 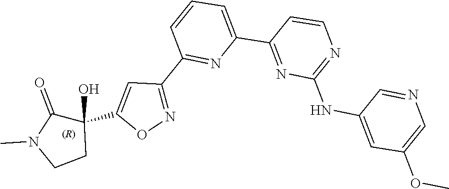 (R)-3-Hydroxy-3-(3-(6-(2-((5- methoxypyridin-3- yl)amino)pyrimidin-4-yl)pyridin- 2-yl)isoxazol-5-yl)-1- methylpyrrolidin-2-one | ¹H NMR (500 MHz, DMSO-d₆) δ 10.05 (s, 1H), 8.77 (d, J = 5.1 Hz, 1H), 8.61 (d, J = 2.1 Hz, 1H), 8.48 (dd, J = 7.7, 1.1 Hz, 1H), 8.24 (t, J = 7.8 Hz, 1H), 8.19 (dd, J = 7.8, 1.1 Hz, 1H), 8.07 (t, J = 2.4 Hz, 1H), 7.97-7.94 (m, 2H), 7.20 (s, 1H), 6.78 (s, 1H), 3.88 (s, 3H), 3.54-3.48 (m, 1H), 3.48-3.43 (m, 1H), 2.86 (s, 3H), 2.67- 2.60 (m, 1H), 2.35- 2.27 (m, 1H). MS (ESI⁺): m/z = 460.4. | RP HPLC (NH₄OH Modifier) | 15 |
| 289 | 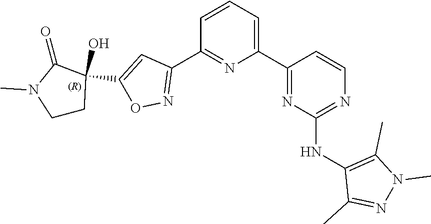 | ¹H NMR (500 MHz, DMSO-d₆) δ 8.60 (s, 1H), 8.52 (s, 1H), 8.13 (s, 2H), 7.71 (d, J = 5.0 Hz, 1H), 7.15 (s, 1H), 6.77 (s, 1H), 3.68 (s, 3H), 3.54-3.48 (m, 1H), 3.48-3.41 (m, 1H), 2.86 (s, 3H), 2.66- 2.59 (m, 1H), 2.34- 2.26 (m, 1H), 2.10 (s, 3H), 2.00 (s, 3H). MS (ESI⁺): m/z = 461.6. | RP HPLC (NH₄OH Modifier) | 15 |
| | (R)-3-Hydroxy-1-methyl-3-(3- | | | |
| | (6-(2-((1,3,5-trimethyl-1H- | | | |
| | pyrazol-4-yl)amino)pyrimidin-4- | | | |
| | yl)pyridin-2-yl)isoxazol-5- | | | |
| | yl)pyrrolidin-2-one | | | |
| 290 | 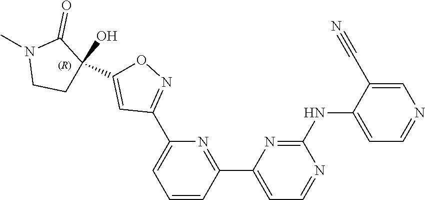 | ¹H NMR (500 MHz, DMSO-d₆) δ 10.25 (s, 1H), 8.90 (d, J = 0.6 Hz, 1H), 8.85 (d, J = 5.1 Hz, 1H), 8.72 (d, J = 5.8 Hz, 1H), 8.46 (dd, J = 7.1, 1.7 Hz, 1H), 8.24-8.20 (m, 1H), 8.20-8.18 (m, 1H), 8.14 (d, J = 5.1 Hz, 1H), 8.01 (dd, J = 5.8, 0.6 Hz, 1H), 7.21 (s, 1H), 6.78 (s, 1H), 3.54- | RP HPLC (NH₄OH Modifier) | 14 |
| | (R)-4-((4-(6-(5-(3-Hydroxy-1- | 3.48 (m, 1H), 3.48- | | |
| | methyl-2-oxopyrrolidin-3- | 3.43 (m, 1H), 2.86 (s, | | |
| | yl)isoxazol-3-yl)pyridin-2- | 4H), 2.67-2.60 (m, | | |
| | yl)pyrimidin-2- | 1H), 2.34-2.28 (m, | | |
| | yl)amino)nicotinonitrile | 1H). MS (ESI⁺): m/z = | | |
| | | 455.1. | | |
| 291 | 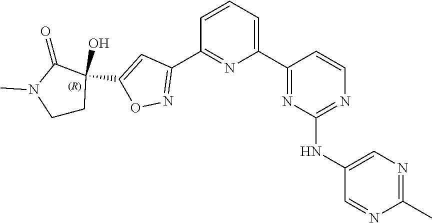 (R)-3-Hydroxy-1-methyl-3-(3- | ¹H NMR (500 MHz, DMSO-d₆) δ 10.05 (s, 1H), 9.14 (s, 2H), 8.78- 8.75 (m, 1H), 8.47- 8.43 (m, 1H), 8.25 (t, J = 7.8 Hz, 1H), 8.20- 8.17 (m, 1H), 7.97 (d, J = 5.1 Hz, 1H), 7.20 (s, 1H), 6.77 (s, 1H), 3.54- 3.48 (m, 1H), 3.48- 3.43 (m, 1H), 2.86 (s, 3H), 2.67-2.61 (m, 1H), 2.60 (s, 3H), 2.35- 2.27 (m, 1H). MS (ESI⁺): m/z = 445.0. | RP HPLC (NH₄OH Modifier) | 37 |
| | (6-(2-((2-methylpyrimidin-5- | | | |
| | yl)amino)pyrimidin-4-yl)pyridin- | | | |
| | 2-yl)isoxazol-5-yl)pyrrolidin-2- | | | |
| | one | | | |
| 292 | 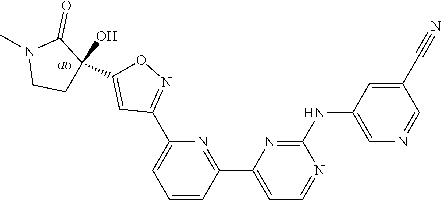 (R)-5-((4-(6-(5-(3-Hydroxy-1- | ¹H NMR (500 MHz, DMSO-d₆) δ 10.42 (s, 1H), 9.23 (d, J = 2.6 Hz, 1H), 8.83 (d, J = 5.1 Hz, 1H), 8.75 (t, J = 2.6, 1.8 Hz, 1H), 8.63 (d, J = 1.9 Hz, 1H), 8.47 (dd, J = 7.7, 1.2 Hz, 1H), 8.25 (t, J = 7.7 Hz, 1H), 8.20 (dd, J = 7.8, 1.2 Hz, 1H), 8.03 (d, J = 5.1 Hz, 1H), 7.20 (s, 1H), 6.77 (s, 1H), 3.54-3.48 (m, | RP HPLC (NH₄OH Modifier) | 11 |
| | methyl-2-oxopyrrolidin-3- | 1H), 3.48-3.43 (m, | | |
| | yl)isoxazol-3-yl)pyridin-2- | 1H), 2.86 (s, 3H), 2.67- | | |
| | yl)pyrimidin-2- | 2.60 (m, 1H), 2.35- | | |
| | yl)amino)nicotinonitrile | 2.28 (m, 1H). MS | | |
| | | (ESI⁺): m/z = 455.1. | | |
| 293 | 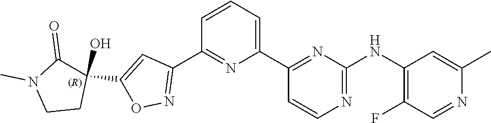 (R)-3-(3-(6-(2-((5-Fluoro-2- methylpyridin-4- yl)amino)pyrimidin-4-yl)pyridin- 2-yl)isoxazol-5-yl)-3-hydroxy-1- methylpyrrolidin-2-one | ¹H NMR (500 MHz, DMSO-d₆) δ 9.75 (s, 1H), 8.82 (d, J = 5.1 Hz, 1H), 8.47 (dd, J = 7.7, 1.2 Hz, 1H), 8.35 (d, J = 3.0 Hz, 1H), 8.24 (t, J = 7.7 Hz, 1H), 8.19 (dd, J = 7.8, 1.2 Hz, 1H), 8.16 (d, J = 6.6 Hz, 1H), 8.06 (d, J = 5.1 Hz, 1H), 7.20 (s, 1H), 6.78 (s, 1H), 3.53- 3.48 (m, 1H), 3.48- 3.43 (m, 1H), 2.86 (s, | RP HPLC (NH₄OH Modifier) | 21 |
| | | 3H), 2.66-2.60 (m, | | |
| | | 1H), 2.49 (s, 3H), 2.34- | | |
| | | 2.28 (m, 1H). MS | | |
| | | (ESI⁺): m/z = 462.1. | | |
| 294 | 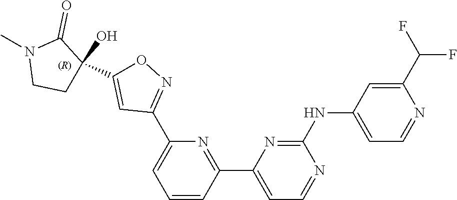 | ¹H NMR (400 MHz, DMSO-d₆) δ 10.52 (s, 1H), 8.79 (d, J = 5.1 Hz, 1H), 8.47-8.45 (m, 1H), 8.45-8.43 (m, 1H), 8.22-8.20 (m, 1H), 8.20-8.15 (m, 1H), 8.15-8.12 (m, 1H), 8.01 (d, J = 5.1 Hz, 1H), 7.94- 7.91 (m, 1H), 7.14 (s, 1H), 6.70 (s, 1H), 3.47- 3.42 (m, 1H), 3.42- | RP HPLC (NH₄OH Modifier) | 9 |
| | (R)-3-(3-(6-(2-((2- | 3.36 (m, 1H), 2.79 (s, | | |
| | (Difluoromethyl)pyridin-4- | 3H), 2.60-2.52 (m, | | |
| | yl)amino)pyrimidin-4-yl)pyridin- | 1H), 2.28-2.20 (m, | | |
| | 2-yl)isoxazol-5-yl)-3-hydroxy-1- | 1H), 1.17 (s, 1H). MS | | |
| | methylpyrrolidin-2-one | (ESI⁺): m/z = 480.1. | | |
| 295 | 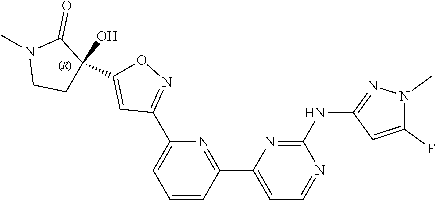 | ¹H NMR (500 MHz, DMSO-d₆) δ 9.99 (s, 1H), 8.66 (d, J = 5.0 Hz, 1H), 8.46 (dd, J = 7.7, 1.2 Hz, 1H), 8.21 (t, J = 7.8 Hz, 1H), 8.16 (dd, J = 7.8, 1.2 Hz, 1H), 7.86 (d, J = 5.0 Hz, 1H), 7.16 (s, 1H), 6.44 (d, J = 5.6 Hz, 1H), 3.65-3.61 (m, 3H), 2.85 (s, 3H), 2.65- 2.59 (m, 2H), 2.33- | RP HPLC (TFA modifier) | 24 |
| | (R)-3-(3-(6-(2-((5-Fluoro-1- | 2.27 (m, 2H). MS | | |
| | methyl-1H-pyrazol-3- | (ESI⁺): m/z = 451.0. | | |
| | yl)amino)pyrimidin-4-yl)pyridin- | | | |
| | 2-yl)isoxazol-5-yl)-3-hydroxy-1- | | | |
| | methylpyrrolidin-2-one | | | |
| 296 | 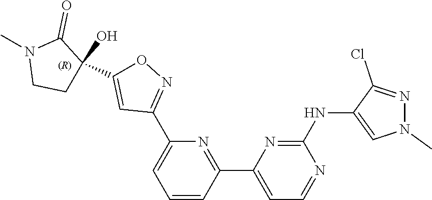 | ¹H NMR (500 MHz, DMSO-d₆) δ 8.95 (s, 1H), 8.62 (d, J = 5.0 Hz, 1H), 8.41 (d, J = 7.4 Hz, 1H), 8.21- 8.17 (m, 1H), 8.17- 8.13 (m, 1H), 8.04 (s, 1H), 7.81 (d, J = 5.0 Hz, 1H), 7.17 (s, 1H), 6.77 (s, 1H), 3.85 (s, 3H), 3.53-3.48 (m, 1H), 3.48-3.42 (m, 1H), 2.86 (s, 3H), 2.66- | RP HPLC (NH₄OH Modifier) | 18 |
| | (R)-3-(3-(6-(2-((3-Chloro-1- | 2.59 (m, 1H), 2.34- | | |
| | methyl-1H-pyrazol-4- | 2.27 (m, 1H). MS | | |
| | yl)amino)pyrimidin-4-yl)pyridin- | (ESI⁺): m/z = 467.1. | | |
| | 2-yl)isoxazol-5-yl)-3-hydroxy-1- | | | |
| | methylpyrrolidin-2-one | | | |
| 297 | 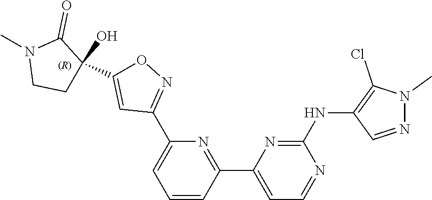 (R)-3-(3-(6-(2-((5-Chloro-1- methyl-1H-pyrazol-4- | ¹H NMR (500 MHz, DMSO-d₆) δ 9.06 (s, 1H), 8.60 (d, J = 5.0 Hz, 1H), 8.41-8.34 (m, 1H), 8.20-8.16 (m, 1H), 8.16-8.13 (m, 1H), 7.81 (d, J = 5.0 Hz, 1H), 7.79 (s, 1H), 7.17 (s, 1H), 3.84 (s, 3H), 3.54-3.48 (m, 1H), 3.48-3.42 (m, 1H), 2.86 (s, 3H), 2.66- 2.59 (m, 1H), 2.34- 2.27 (m, 1H). MS (ESI⁺): m/z = 467.1. | RP HPLC (TFA modifier) | 30 |
| | yl)amino)pyrimidin-4-yl)pyridin- | | | |
| | 2-yl)isoxazol-5-yl)-3-hydroxy-1- | | | |
| | methylpyrrolidin-2-one | | | |
| 298 | 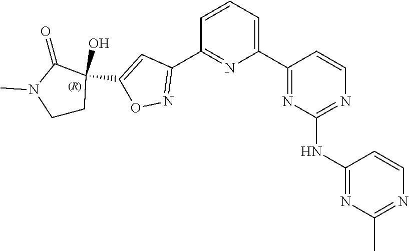 | ¹H NMR (500 MHz, DMSO-d₆) δ 11.54 (s, 1H), 8.95 (d, J = 5.1 Hz, 1H), 8.69 (d, J = 6.6 Hz, 1H), 8.55 (dd, J = 7.4, 1.4 Hz, 1H), 8.47 (d, J = 6.7 Hz, 1H), 8.27-8.24 (m, 2H), 8.24-8.21 (m, 1H), 7.22 (s, 1H), 3.54- 3.49 (m, 1H), 3.49- 3.43 (m, 1H), 2.86 (s, 3H), 2.67-2.60 (m, 4H), 2.35-2.28 (m, 1H). MS (ESI⁺): m/z = 445.0. | RP HPLC (TFA modifier) | 23 |
| | (R)-3-Hydroxy-1-methyl-3-(3- | | | |
| | (6-(2-((2-methylpyrimidin-4- | | | |
| | yl)amino)pyrimidin-4-yl)pyridin- | | | |
| | 2-yl)isoxazol-5-yl)pyrrolidin-2- | | | |
| | one | | | |
| 299 | 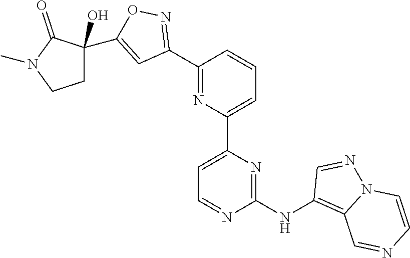 [SMILES: CN1CC[C@@](O)(c2cc(-c3cccc(-c4ccnc(Nc5cnn6ccncc56)n4)n3)no2)C1=O] (R)-3-Hydroxy-1-methyl-3-(3- (6-(2-(pyrazolo[1,5-a]pyrazin-3- ylamino)pyrimidin-4-yl)pyridin- 2-yl)isoxazol-5-yl)pyrrolidin-2-one | ¹H NMR (400 MHz, DMSO-d₆) δ 10.26 (s, 1H), 9.43 (d, J = 1.6 Hz, 1H), 8.74 (d, J = 5.2 Hz, 1H), 8.70 (s, 1H), 8.64 (dd, J = 1.6, 4.8 Hz, 1H), 8.51-8.43 (m, 1H), 8.26-8.15 (m, 2H), 7.90 (d, J = 5.2 Hz, 1H), 7.81 (d, J = 4.8 Hz, 1H), 7.19 (s, 1H), 6.80 (s, 1H), 3.55- 3.45 (m, 2H), 2.86 (s, 3H), 2.71-2.58 (m, 1H), 2.37-2.24 (m,NH3 in 1H). MS (ESI⁺): m/z = 470.2. | HPLC: Welch Xtimate C18 150 × 30 mm, 5 μm), 25- 55% MeCN/water (0.05% NH₃ in H₂O + 10 mM NH₄HCO₃); then followed by further purification by SFC: DAICEL CHIRALC EL OD-H | 6 |
| | | | column, 5 | |
| | | | μm, 250 × | |
| | | | 30 mm; | |
| | | | 55% (v/v) | |
| | | | EtOH | |
| | | | (containing | |
| | | | 0.1% NH₃ | |
| | | | in H₂O)/ | |
| | | | 45% CO₂ | |
| 300 | 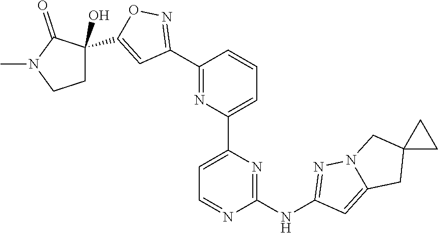 [SMILES: CN1CC[C@@](O)(c2cc(-c3cccc(-c4ccnc(Nc5cc6n(n5)CC5(CC5)C6)n4)n3)no2)C1=O] (R)-3-(3-(6-(2-((4′H,6′H- | ¹H NMR (400 MHz, DMSO-d₆) δ 9.88 (s, 1H), 8.66-8.62 (m, 1H), 8.52-8.44 (m, 1H), 8.25-8.11 (m, 2H), 7.86-7.77 (m, 1H), 7.18 (s, 1H), 6.79 (s, 1H), 6.54 (s, 1H), 3.96 (s, 2H), 3.54- 3.41 (m, 2H), 2.91 (s, 2H), 2.85 (s, 3H), 2.67- 2.58 (m, 1H), 2.36- 2.25 (m, 1H), 0.89- 0.73 (m, 4H) MS (ESI+): m/z = 485.3 [M + H]⁺. | RP HPLC (NH₄OH modifier) | 45 |
| | Spiro[cyclopropane-1,5′- | | | |
| | pyrrolo[1,2-b]pyrazol]-2′- | | | |
| | yl)amino)pyrimidin-4-yl)pyridin- | | | |
| | 2-yl)isoxazol-5-yl)-3-hydroxy-1- | | | |
| | methylpyrrolidin-2-one | | | |
| 301 | 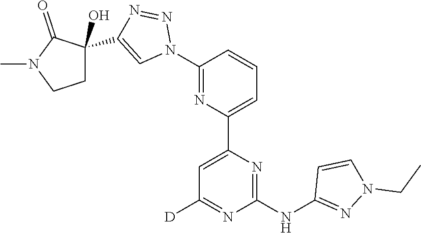 [SMILES: [2H]c1cc(-c2cccc(-n3cc([C@]4(O)CCN(C)C4=O)nn3)n2)nc(Nc2ccn(CC)n2)n1] (R)-3-(1-(6-(2-((1-Ethyl-1H- pyrazol-3-yl)amino)pyrimidin-4- | ¹H NMR (400 MHz, DMSO-d₆) δ 9.96 (s, 1H), 8.95 (s, 1H), 8.49- 8.44 (m, 1H), 8.39- 8.32 (m, 1H), 8.30- 8.24 (m, 1H), 7.82 (s, 1H), 7.67 (d, J = 2.0 Hz, 1H), 6.73 (d, J = 2.0 Hz, 1H), 6.37 (s, 1H), 4.07 (q, J = 7.2 Hz, 2H), 3.50-3.49 (m, 2H), 2.83 (s, 3H), 2.82- 2.75 (m, 1H), 2.34- 2.24 (m, 1H), 1.38 (t, J = 7.2 Hz, 3H). MS (ESI+): m/z = 448.2 [M + H]⁺. | HPLC ((Phenomenex C18 75 × 30 mm, 3 μm) 20- 50% MeCN and water (with 0.05% NH₃ in H₂O + 10 mM NH₄HCO₃)) then followed by further purification by SFC | 16 |
| | yl-6-d)pyridin-2-yl)-1H-1,2,3- | | (DAICEL | |
| | triazol-4-yl)-3-hydroxy-1- | | CHIRALP | |
| | methylpyrrolidin-2-one | | AK AD | |
| | | | column, 10 | |
| | | | μm, 250 × | |
| | | | 30 mm; | |
| | | | 55% (v/v) | |
| | | | iPrOH | |
| | | | (containing | |
| | | | 0.1% of | |
| | | | 25% aq. | |
| | | | NH₃)/CO₂)) | |
| 302 | 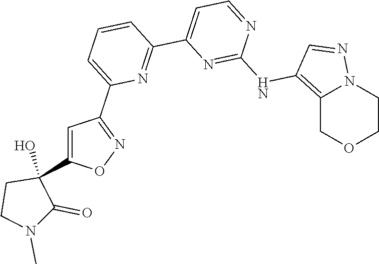 [SMILES: CN1CC[C@@](O)(c2cc(-c3cccc(-c4ccnc(Nc5cnn6c5COCC6)n4)n3)no2)C1=O] | ¹H NMR (400 MHz, DMSO-d₆) δ 9.16 (br. s., 1H), 8.64-8.53 (m, 1H), 8.47-8.29 (m, 1H), 8.24-8.08 (m, 2H), 7.83-7.66 (m, 2H), 7.17 (s, 1H), 6.78 (br. s., 1H), 4.80 (s, 2H), 4.15-4.03 (m, 4H), 3.54-3.41 (m, 2H), 2.85 (s, 3H), 2.68- 2.57 (m, 1H), 2.34- 2.23 (m, 1H). MS (ESI+): m/z = 475.2 [M + H]⁺. | RP HPLC (NH₄OH modifier) | 12 |
| | (R)-3-(3-(6-(2-((6,7-Dihydro-4H- | | | |
| | pyrazolo[5,1-c][1,4]oxazin-3- | | | |
| | yl)amino)pyrimidin-4-yl)pyridin- | | | |
| | 2-yl)isoxazol-5-yl)-3-hydroxy-1- | | | |
| | methylpyrrolidin-2-one | | | |
| 303 | 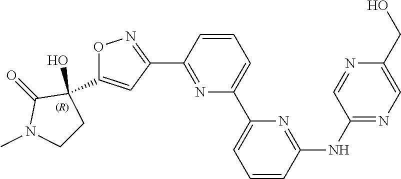 | ¹H NMR (401 MHz, DMSO-d₆) δ 10.42 (s, 1H), 9.57-9.49 (m, 1H), 8.84-8.78 (m, 1H), 8.53-8.46 (m, 1H), 8.44-8.39 (m, 1H), 8.29-8.22 (m, 1H), 8.22-8.14 (m, 1H), 8.06-8.01 (m, 1H), 7.26-7.19 (m, 2H), 7.10 (s, 1H), 6.97 (s, 1H), 6.87-6.68 (m, | RP HPLC (TFA modifier) | 7 |
| | (R)-3-Hydroxy-3-(3-(6-(2-((5- | 1H), 4.61 (s, 2H), 2.86 | | |
| | (hydroxymethyl)pyrazin-2- | (s, 4H), 2.69-2.59 (m, | | |
| | yl)amino)pyrimidin-4-yl)pyridin- | 1H), 2.35-2.26 (m, | | |
| | 2-yl)isoxazol-5-yl)-1- | 1H). MS (ESI⁺): m/z = | | |
| | methylpyrrolidin-2-one | 461.6. | | |
| 304 | 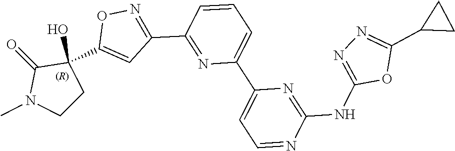 | ¹H NMR (401 MHz, DMSO-d₆) δ 11.26 (s, 1H), 8.83-8.77 (m, 1H), 8.52-8.45 (m, 1H), 8.26-8.15 (m, 2H), 8.11-8.03 (m, 1H), 7.20 (s, 1H), 6.77 (s, 1H), 2.85 (s, 3H), 2.70-2.57 (m, 1H), 2.36-2.25 (m, 1H), | RP HPLC (TFA modifier) | 5 |
| | (R)-3-(3-(6-(2-((5-Cyclopropyl- | 2.25-2.17 (m, 1H), | | |
| | 1,3,4-oxadiazol-2- | 1.17-1.09 (m, 2H), | | |
| | yl)amino)pyrimidin-4-yl)pyridin- | 1.04-0.98 (m, 2H). | | |
| | 2-yl)isoxazol-5-yl)-3-hydroxy-1- | MS (ESI⁺): m/z = | | |
| | methylpyrrolidin-2-one | 461.5. | | |
| 305 | 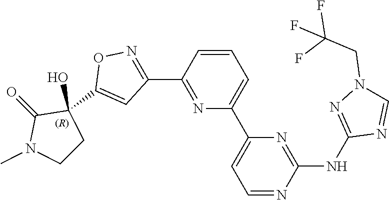 | ¹H NMR (401 MHz, DMSO-d₆) δ 10.17 (s, 1H), 8.72-8.65 (m, 1H), 8.56 (s, 1H), 8.54- 8.48 (m, 1H), 8.18- 8.12 (m, 2H), 7.94- 7.90 (m, 1H), 7.18 (s, 1H), 5.33-5.22 (m, 2H), 2.85 (s, 3H), 2.66- 2.58 (m, 1H), 2.34- 2.25 (m, 1H). MS (ESI⁺): m/z = 502.7. | RP HPLC (TFA modifier) | 7 |
| | (R)-3-Hydroxy-1-methyl-3-(3- | | | |
| | (6-(2-((1-(2,2,2-trifluoroethyl)- | | | |
| | 1H-1,2,4-triazol-3- | | | |
| | yl)amino)pyrimidin-4-yl)pyridin- | | | |
| | 2-yl)isoxazol-5-yl)pyrrolidin-2- | | | |
| | one | | | |
| 306 | 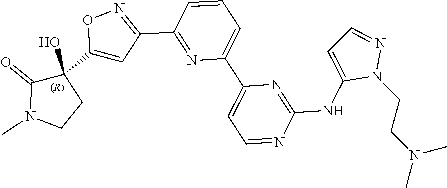 (R)-3-(3-(6-(2-((1-(2- (Dimethylamino)ethyl)-1H- pyrazol-5-yl)amino)pyrimidin-4- yl)pyridin-2-yl)isoxazol-5-yl)-3- | ¹H NMR (401 MHz, DMSO-d₆) δ 9.78 (s, 1H), 9.50 (s, 1H), 8.74- 8.68 (m, 1H), 8.42- 8.33 (m, 1H), 8.23- 8.11 (m, 2H), 7.99- 7.92 (m, 1H), 7.61- 7.55 (m, 1H), 7.18 (s, 1H), 6.51-6.45 (m, 1H), 4.49-4.43 (m, 3H), 3.61-3.54 (m, 2H), 3.54-3.41 (m, 3H), 2.87-2.84 (m, 3H), 2.67-2.57 (m, 1H), 2.36-2.25 (m, 1H). MS (ESI⁺): m/z = 490.6. | RP HPLC (TFA modifier) | 13 |
| | hydroxy-1-methylpyrrolidin-2-one | | | |
| 307 | 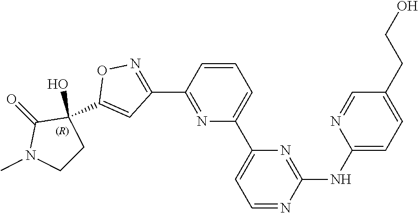 | ¹H NMR (401 MHz, DMSO-d₆) δ 11.58 (s, 1H), 8.93-8.87 (m, 1H), 8.56-8.48 (m, 1H), 8.33-8.29 (m, 1H), 8.29-8.17 (m, 3H), 8.16-8.09 (m, 1H), 7.89-7.81 (m, 1H), 7.21 (s, 1H), 3.71- 3.63 (m, 2H), 3.56- 3.41 (m, 2H), 2.87 (s, 3H), 2.84-2.75 (m, 2H), 2.68-2.58 (m, 1H), 2.37-2.27 (m, | RP HPLC (TFA modifier) | 23 |
| | (R)-3-(3-(6-(2-((5,6-Dihydro-4H- | 1H). MS (ESI⁺): m/z = | | |
| | pyrrolo[1,2-b]pyrazol-3- | 474.4. | | |
| | yl)amino)pyrimidin-4-yl)pyridin- | | | |
| | 2-yl)isoxazol-5-yl)-3-hydroxy-1- | | | |
| | methylpyrrolidin-2-one | | | |
| 308 | 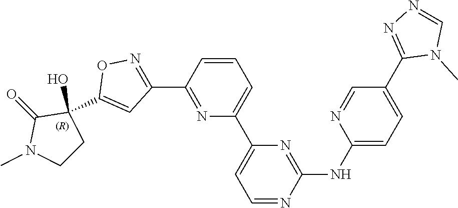 (R)-3-Hydroxy-1-methyl-3-(3- (6-(2-((5-(4-methyl-4H-1,2,4- | ¹H NMR (401 MHz, DMSO-d₆) δ 10.46 (s, 1H), 8.87-8.80 (m, 1H), 8.75-8.68 (m, 1H), 8.65 (s, 1H), 8.60- 8.54 (m, 2H), 8.29- 8.17 (m, 3H), 8.09- 8.03 (m, 1H), 7.27- 7.18 (m, 2H), 7.10 (s, 1H), 6.98 (s, 1H), 6.78 (s, 1H), 3.82 (s, 4H), 2.86 (s, 3H), 2.69- 2.58 (m, 1H), 2.37- 2.24 (m, 1H). MS (ESI⁺): m/z = 511.5. | RP HPLC (NH₄OH Modifier) | 6 |
| | triazol-3-yl)pyridin-2- | | | |
| | yl)amino)pyrimidin-4-yl)pyridin- | | | |
| | 2-yl)isoxazol-5-yl)pyrrolidin-2- | | | |
| | one | | | |
| 309 | 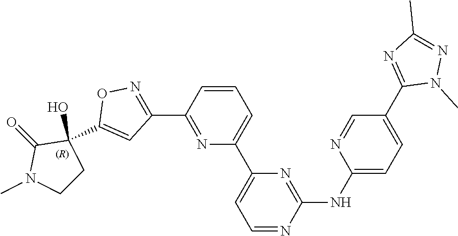 | 1H NMR (401 MHz, DMSO-d₆) δ 8.73- 8.68 (m, 1H), 8.34- 8.28 (m, 1H), 8.23- 8.14 (m, 2H), 8.02- 7.97 (m, 1H), 7.18 (s, 1H), 3.55-3.39 (m, 3H), 2.85 (s, 3H), 2.67- 2.57 (m, 1H), 2.34- 2.26 (m, 1H), 2.25 (s, 3H). MS (ESI⁺): m/z = 448.5. | RP HPLC (NH₄OH Modifier) | 11 |
| | (R)-3-(3-(6-(2-((5-(1,3- | | | |
| | Dimethyl-1H-1,2,4-triazol-5- | | | |
| | yl)pyridin-2-yl)amino)pyrimidin- | | | |
| | 4-yl)pyridin-2-yl)isoxazol-5-yl)- | | | |
| | 3-hydroxy-1-methylpyrrolidin-2- | | | |
| | one | | | |
| 310 | 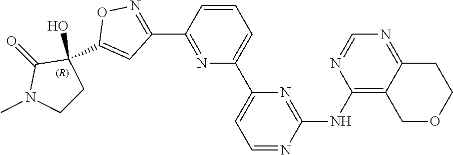 (R)-3-(3-(6-(2-((7,8-Dihydro-5H- pyrano[4,3-d]pyrimidin-4- yl)amino)pyrimidin-4-yl)pyridin- 2-yl)isoxazol-5-yl)-3-hydroxy-1- | ¹H NMR (401 MHz, DMSO-d₆) δ 8.86- 8.80 (m, 1H), 8.78 (s, 1H), 8.43-8.36 (m, 1H), 8.22-8.14 (m, 2H), 8.14-8.09 (m, 1H), 7.20 (s, 1H), 4.66 (s, 2H), 4.03-3.96 (m, 3H), 3.54-3.41 (m, 2H), 2.96-2.88 (m, 2H), 2.86 (s, 3H), 2.69- 2.59 (m, 1H), 2.35- 2.25 (m, 1H). MS (ESI⁺): m/z = 448.5. | RP HPLC (TFA modifier) | 8 |
| | methylpyrrolidin-2-one | | | |
| 311 | 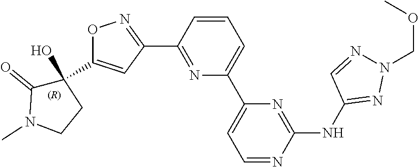 | ¹H NMR (401 MHz, DMSO-d₆) δ 10.58 (s, 1H), 8.76-8.71 (m, 1H), 8.54-8.49 (m, 1H), 8.30 (s, 1H), 8.26- 8.15 (m, 2H), 7.96- 7.92 (m, 1H), 7.19 (s, 1H), 6.78 (s, 1H), 5.57 (s, 2H), 3.55-3.41 (m, 2H), 2.85 (s, 3H), 2.69- 2.58 (m, 1H), 2.35- | RP HPLC (NH₄OH Modifier) | 5 |
| | (R)-3-Hydroxy-3-(3-(6-(2-((2- | 2.25 (m, 1H). MS | | |
| | (methoxymethyl)-2H-1,2,3- | (ESI⁺): m/z = 464.6. | | |
| | triazol-4-yl)amino)pyrimidin-4- | | | |
| | yl)pyridin-2-yl)isoxazol-5-yl)-1- | | | |
| | methylpyrrolidin-2-one | | | |
| 312 | 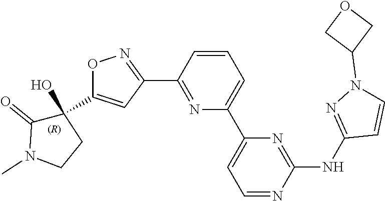 (R)-3-Hydroxy-1-methyl-3-(3- | ¹H NMR (401 MHz, DMSO-d₆) δ 10.18 (s, 1H), 8.70-8.63 (m, 1H), 8.51-8.45 (m, 1H), 8.25-8.13 (m, 2H), 7.89-7.83 (m, 1H), 7.83-7.78 (m, 1H), 7.18 (s, 1H), 6.85- 6.79 (m, 1H), 5.57- 5.49 (m, 2H), 4.97- 4.88 (m, 3H), 3.54- 3.41 (m, 2H), 2.86 (s, 3H), 2.67-2.58 (m, 1H), 2.35-2.26 (m, | RP HPLC (TFA modifier) | 19 |
| | (6-(2-((1-(oxetan-3-yl)-1H- | 1H). MS (ESI⁺): m/z = | | |
| | pyrazol-3-yl)amino)pyrimidin-4- | 475.4. | | |
| | yl)pyridin-2-yl)isoxazol-5- | | | |
| | yl)pyrrolidin-2-one | | | |
| 313 | 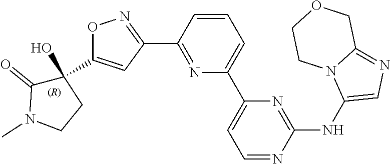 | ¹H NMR (401 MHz, DMSO-d₆) δ 9.68 (s, 1H), 8.73-8.66 (m, 1H), 8.42-8.33 (m, 1H), 8.23-8.11 (m, 2H), 7.97-7.91 (m, 1H), 7.27 (s, 1H), 7.18 (s, 2H), 7.05 (s, 1H), 6.79 (s, 1H), 4.89 (s, 2H), 4.11-4.04 (m, 2H), 3.99-3.92 (m, | RP HPLC (NH₄OH Modifier) | 22 |
| | (R)-3-(3-(6-(2-((5,6-Dihydro-8H- | 2H), 3.54-3.40 (m, | | |
| | imidazo[2,1-c][1,4]oxazin-3- | 6H), 2.85 (s, 4H), 2.67- | | |
| | yl)amino)pyrimidin-4-yl)pyridin- | 2.57 (m, 1H), 2.36- | | |
| | 2-yl)isoxazol-5-yl)-3-hydroxy-1- | 2.25 (m, 1H). MS | | |
| | methylpyrrolidin-2-one | (ESI⁺): m/z = 475.2. | | |
| 314 | 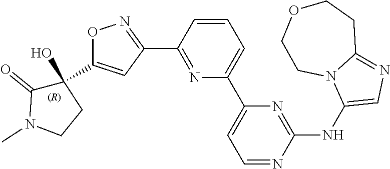 (R)-3-Hydroxy-1-methyl-3-(3- (6-(2-((5,6,8,9- tetrahydroimidazo[1,2- d][1,4]oxazepin-3- yl)amino)pyrimidin-4-yl)pyridin- | ¹H NMR (401 MHz, DMSO-d₆) δ 9.95 (s, 1H), 8.75-8.67 (m, 1H), 8.43-8.34 (m, 1H), 8.23-8.14 (m, 2H), 8.04-7.98 (m, 1H), 7.62 (s, 1H), 7.18 (s, 1H), 4.24-4.17 (m, 3H), 3.95-3.88 (m, 2H), 3.88-3.82 (m, 3H), 3.54-3.41 (m, 3H), 3.41-3.33 (m, 2H), 2.85 (s, 4H), 2.66- 2.57 (m, 1H), 2.36- 2.25 (m, 1H). MS (ESI⁺): m/z = 489.5. | RP HPLC (TFA modifier) | 19 |
| | 2-yl)isoxazol-5-yl)pyrrolidin-2- | | | |
| | one | | | |
| 315 | 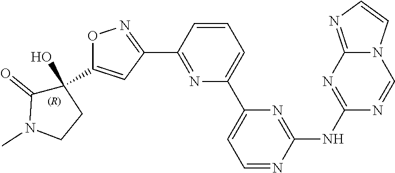 (R)-3-Hydroxy-3-(3-(6-(2- (imidazo[1,2-a][1,3,5]triazin-2- | ¹H NMR (401 MHz, DMSO-d₆) δ 9.00- 8.95 (m, 1H), 8.58- 8.52 (m, 1H), 8.34 (s, 1H), 8.31-8.26 (m, 1H), 8.25-8.18 (m, 2H), 8.08-8.01 (m, 1H), 7.70-7.65 (m, 1H), 7.22 (s, 1H), 3.55- 3.40 (m, 3H), 2.86 (s, 3H), 2.68-2.57 (m, 1H), 2.37-2.26 (m, 1H). MS (ESI⁺): m/z = 471.5 | RP HPLC (TFA modifier) | 11 |
| | ylamino)pyrimidin-4-yl)pyridin- | | | |
| | 2-yl)isoxazol-5-yl)-1- | | | |
| | methylpyrrolidin-2-one | | | |
| 316 | 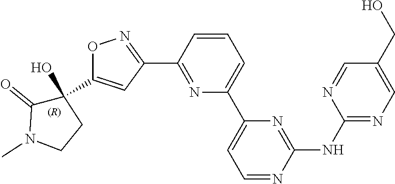 | ¹H NMR (401 MHz, MeOH-d₄) δ 8.80- 8.78 (m, 1H), 8.66- 8.62 (m, 3H), 8.21- 8.11 (m, 2H), 7.24 (s, 1H), 3.66-3.57 (m, 2H), 3.00 (s, 3H), 2.83- 2.76 (m, 2H), 2.49- 2.42 (m, 2H). MS (ESI⁺): m/z = 461.2. | RP HPLC (NH₄OH Modifier) | 18 |
| | (R)-3-Hydroxy-3-(3-(6-(2-((5- | | | |
| | (hydroxymethyl)pyrimidin-2- | | | |
| | yl)amino)pyrimidin-4-yl)pyridin- | | | |
| | 2-yl)isoxazol-5-yl)-1- | | | |
| | methylpyrrolidin-2-one | | | |
| 317 | 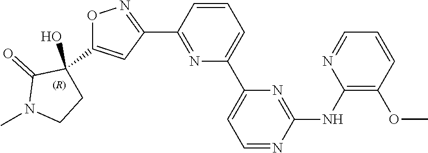 (R)-3-Hydroxy-3-(3-(6-(2-((3- methoxypyridin-2- yl)amino)pyrimidin-4-yl)pyridin- | ¹H NMR (500 MHz, DMSO-d₆) δ 8.86- 8.81 (m, 1H), 8.39- 8.35 (m, 1H), 8.23- 8.18 (m, 1H), 8.06- 8.02 (m, 1H), 7.17 (s, 1H), 3.52-3.41 (m, 2H), 2.85 (s, 3H), 2.64 (s, 3H), 2.62-2.57 (m, 1H), 2.56-2.53 (m, 3H), 2.33-2.26 (m, 1H). MS (ESI⁺): m/z = 460.7. | RP HPLC (TFA modifier) | 52 |
| | 2-yl)isoxazol-5-yl)-1- | | | |
| | methylpyrrolidin-2-one | | | |
| 318 | 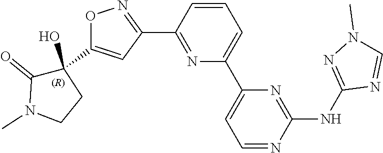 (R)-3-Hydroxy-1-methyl-3-(3- (6-(2-((1-methyl-1H-1,2,4- | ¹H NMR (500 MHz, DMSO-d₆) δ 9.93 (s, 1H), 8.66-8.64 (m, 1H), 8.48-8.44 (m, 1H), 8.37 (s, 1H), 8.19- 8.13 (m, 2H), 7.89- 7.87 (m, 1H), 7.17 (s, 1H), 6.75 (s, 1H), 3.86 (s, 3H), 2.85 (s, 3H), 2.65-2.59 (m, 2H), 2.38-2.26 (m, 2H). MS (ESI⁺): m/z = 434.6 | RP HPLC (TFA modifier) | 13 |
| | triazol-3-yl)amino)pyrimidin-4- | | | |
| | yl)pyridin-2-yl)isoxazol-5- | | | |
| | yl)pyrrolidin-2-one | | | |
| 319 | 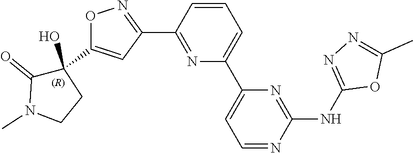 (R)-3-Hydroxy-1-methyl-3-(3- | ¹H NMR (500 MHz, DMSO-d₆) δ 11.28 (s, 1H), 8.81-8.78 (m, 1H), 8.52-8.49 (m, 1H), 8.24-8.17 (m, 2H), 8.10-8.08 (m, 1H), 7.20 (s, 1H), 6.76 (s, 1H), 2.85 (s, 3H), 2.66-2.59 (m, 2H), 2.38-2.26 (m, 2H). MS (ESI⁺): m/z = 436.2 | RP HPLC (TFA modifier) | 6 |
| | (6-(2-((5-methyl-1,3,4- | | | |
| | oxadiazol-2-yl)amino)pyrimidin- | | | |
| | 4-yl)pyridin-2-yl)isoxazol-5- | | | |
| | yl)pyrrolidin-2-one | | | |
| 320 | 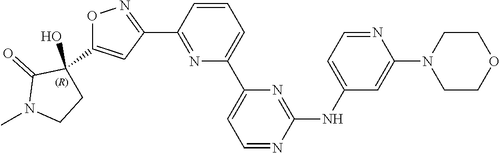 | ¹H NMR (500 MHz, DMSO-d₆) δ 10.05 (s, 1H), 7.20-7.16 (m, 2H), 6.77 (s, 1H), 3.77- 3.70 (m, 4H), 3.54- 3.45 (m, 2H), 3.45- 3.40 (m, 5H), 2.85 (s, 3H), 2.66-2.59 (m, 2H), 2.37-2.27 (m, 2H). MS (ESI⁺): m/z = 515.4. | RP HPLC (NH₄OH Modifier) | 5 |
| | (R)-3-Hydroxy-1-methyl-3-(3- | | | |
| | (4-methyl-6-(2-((2- | | | |
| | morpholinopyridin-4- | | | |
| | yl)amino)pyrimidin-4-yl)pyridin- | | | |
| | 2-yl)isoxazol-5-yl)pyrrolidin-2- | | | |
| | one | | | |
| 321 | 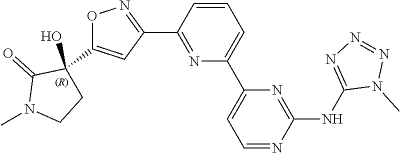 (R)-3-Hydroxy-1-methyl-3-(3- (6-(2-((1-methyl-1H-tetrazol-5- | ¹H NMR (500 MHz, DMSO-d₆) δ 10.65 (s, 1H), 8.81-8.72 (m, 1H), 8.36-8.28 (m, 1H), 8.24-8.13 (m, 2H), 8.10-8.05 (m, 1H), 7.18 (s, 1H), 6.97 (s, 1H), 3.94 (s, 3H), 2.85 (s, 3H), 2.66- 2.58 (m, 1H), 2.34- 2.26 (m, 1H). MS (ESI⁺): m/z = 435.2. | RP HPLC (TFA modifier) | 19 |
| | yl)amino)pyrimidin-4-yl)pyridin- | | | |
| | 2-yl)isoxazol-5-yl)pyrrolidin-2- | | | |
| | one | | | |
| 322 | 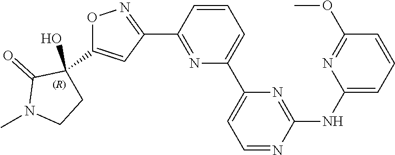 (R)-3-Hydroxy-3-(3-(6-(2-((6- methoxypyridin-2- yl)amino)pyrimidin-4-yl)pyridin- 2-yl)isoxazol-5-yl)-1- | ¹H NMR (500 MHz, MeOH-d₄) δ 8.77- 8.71 (m, 1H), 8.62- 8.56 (m, 1H), 8.23- 8.18 (m, 1H), 8.18- 8.11 (m, 2H), 7.83- 7.67 (m, 2H), 7.21 (s, 1H), 6.58-6.51 (m, 1H), 3.98 (s, 3H), 3.65- 3.54 (m, 2H), 2.98 (s, 3H), 2.82-2.73 (m, 1H), 2.47-2.39 (m, 1H). MS (ESI⁺): m/z = 460.2. | RP HPLC (TFA modifier) | 30 |
| | methylpyrrolidin-2-one | | | |
| 323 | 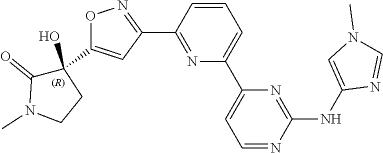 (R)-3-Hydroxy-1-methyl-3-(3- (6-(2-((1-methyl-1H-imidazol-4- yl)amino)pyrimidin-4-yl)pyridin- | 1H NMR (500 MHz, MeOH-d₄) 8.73-8.68 (m, 1H), 8.55-8.48 (m, 2H), 8.21-8.15 (m, 1H), 8.14-8.08 (m, 1H), 8.08-8.04 (m, 1H), 7.45-7.39 (m, 1H), 7.19 (s, 1H), 3.65-3.50 (m, 3H), 3.34 (s, 5H), 2.98 (s, 4H), 2.82-2.73 (m, 1H), 2.47-2.39 (m, 1H). MS (ESI⁺): m/z = 433.1. | RP HPLC (TFA modifier) | 7 |
| | 2-yl)isoxazol-5-yl)pyrrolidin-2- | | | |
| | one | | | |
| 324 | 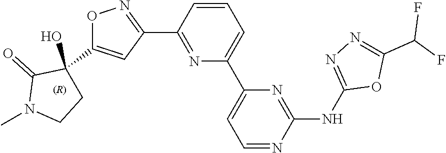 (R)-3-(3-(6-(2-((5- (Difluoromethyl)-1,3,4- oxadiazol-2-yl)amino)pyrimidin- | ¹H NMR (500 MHz, MeOH-d₄) δ 8.82-8.78 (m, 1H), 8.67-8.62 (m, 1H), 8.26-8.20 (m, 1H), 8.20-8.16 (m, 1H), 8.13-8.07 (m, 1H), 7.24-7.18 (m, 1H), 3.63-3.56 (m, 2H), 3.34 (s, 1H), 2.98 (s, 3H), 2.81- 2.72 (m, 1H), 2.46- 2.38 (m, 1H). MS (ESI⁺): m/z = 471.1. | RP HPLC (TFA modifier) | 5 |
| | 4-yl)pyridin-2-yl)isoxazol-5-yl)- | | | |
| | 3-hydroxy-1-methylpyrrolidin-2- | | | |
| | one | | | |
| 325 | 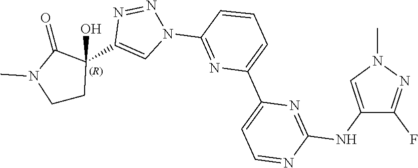 | ¹H NMR (500 MHz, DMSO-d₆) δ 9.17 (s, 1H), 8.93 (s, 1H), 8.68- 8.58 (m, 1H), 8.44- 8.36 (m, 1H), 8.36- 8.28 (m, 1H), 8.28- 8.23 (m, 1H), 7.92- 7.83 (m, 1H), 7.83- 7.76 (m, 1H), 3.76 (s, 3H), 3.52-3.46 (m, 2H), 2.87-2.72 (m, | RP HPLC (TFA modifier) | 4 |
| | (R)-3-(1-(6-(2-((3-Fluoro-1- | 4H), 2.33-2.23 (m, | | |
| | methyl-1H-pyrazol-4- | 1H). MS (ESI⁺): m/z = | | |
| | yl)amino)pyrimidin-4-yl)pyridin- | 451.2. | | |
| | 2-yl)-1H-1,2,3-triazol-4-yl)-3- | | | |
| | hydroxy-1-methylpyrrolidin-2- | | | |
| | one | | | |
| 326 | 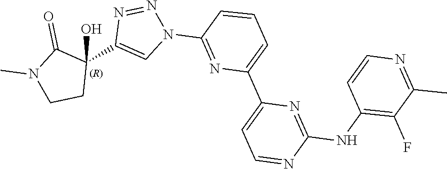 | ¹H NMR (500 MHz, MeOH-d₄) δ 9.25- 9.20 (m, 1H), 8.99 (s, 1H), 8.95-8.91 (m, 1H), 8.63-8.59 (m, 1H), 8.44-8.40 (m, 1H), 8.37-8.34 (m, 1H), 8.32-8.27 (m, 2H), 3.68-3.56 (m, 2H), 2.97 (s, 4H), 2.73- 2.67 (m, 3H), 2.49- | RP HPLC (TFA modifier) | 37 |
| | (R)-3-(1-(6-(2-((3-Fluoro-2- | 2.40 (m, 1H). MS | | |
| | methylpyridin-4- | (ESI⁺): m/z = 462.2. | | |
| | yl)amino)pyrimidin-4-yl)pyridin- | | | |
| | 2-yl)-1H-1,2,3-triazol-4-yl)-3- | | | |
| | hydroxy-1-methylpyrrolidin-2- | | | |
| | one | | | |
| 327 | 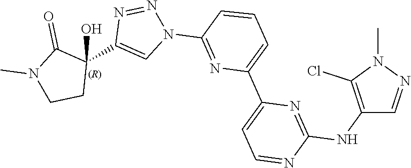 | ¹H NMR (400 MHz, MeOH-d₄) δ 8.86 (s, 1H), 8.46 (m, 1H), 8.39 (m, 1H), 8.20-8.09 (m, 2H), 7.79-7.75 (m, 2H), 3.79 (s, 3H), 3.58-3.47 (m, 2H), 2.91-2.79 (m, 4H), 2.35 (m, 1H). MS (ESI⁺): m/z = 467.1. | RP HPLC (TFA modifier) | 9 |
| | (R)-3-(1-(6-(2-((5-Chloro-1- | | | |
| | methyl-1H-pyrazol-4- | | | |
| | yl)amino)pyrimidin-4-yl)pyridin- | | | |
| | 2-yl)-1H-1,2,3-triazol-4-yl)-3- | | | |
| | hydroxy-1-methylpyrrolidin-2- | | | |
| | one | | | |
| 328 | 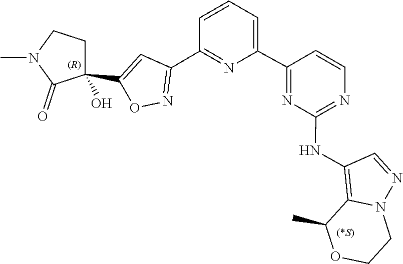 | ¹H NMR (400 MHz, DMSO-d₆) δ 9.00 (br s, 1H), 8.62-8.55 (m, 1H), 8.34 (br s, 1H), 8.21-8.11 (m, 2H), 7.78-7.73 (m, 1H), 7.56 (br s, 1H), 7.16 (s, 1H), 6.77 (br s, 1H), 5.01-4.92 (m, 1H), 4.25-4.17 (m, 1H), 4.14-4.05 (m, 2H), 3.99-3.90 (m, 1H), 3.51-3.41 (m, 2H), 2.85 (s, 3H), 2.66- 2.57 (m, 1H), 2.35- 2.25 (m, 1H), 1.32- 1.28 (m, 3H). MS (ESI): | RP HPLC (NH₄OH Modifier) | 19 |
| | (R)-3-Hydroxy-1-methyl-3-(3- | Mass calcd. for | | |
| | (6-(2-(((*S)-4-methyl-6,7- | C₂₄H₂₄N₈O₄, 488.5; m/z | | |
| | dihydro-4H-pyrazolo[5,1- | found, 489.2 [M + H]⁺. | | |
| | c][1,4]oxazin-3- | | | |
| | yl)amino)pyrimidin-4-yl)pyridin- | | | |
| | 2-yl)isoxazol-5-yl)pyrrolidin-2- | | | |
| | one | | | |
| 329 | 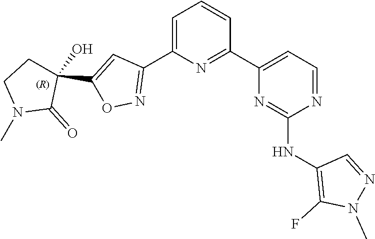 (R)-3-(3-(6-(2-((5-Fluoro-1- | ¹H NMR (400 MHz, DMSO-d₆) δ 8.78 (br s, 1H), 8.61-8.54 (m, 1H), 8.40-8.33 (m, 1H), 8.20-8.07 (m, 2H), 7.81-7.72 (m, 1H), 7.60-7.49 (m, 1H), 7.12 (s, 1H), 3.75 (s, 3H), 3.55-3.41 (m, 2H), 2.87 (s, 3H), 2.69- 2.59 (m, 1H), 2.38- 2.28 (m, 1H). MS (ESI): Mass calcd. for C₂₁H₁₉FN₈O₃, 450.4; m/z found, 451.2 [M + H]⁺. | RP HPLC (NH⁴OH Modifier) | 57 |
| | methyl-1H-pyrazol-4- | | | |
| | yl)amino)pyrimidin-4-yl)pyridin- | | | |
| | 2-yl)isoxazol-5-yl)-3-hydroxy-1- | | | |
| | methylpyrrolidin-2-one | | | |
| 330 | 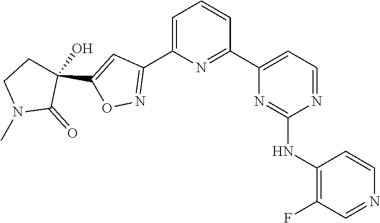 [SMILES: CN1CC[C@@](O)(c2cc(-c3cccc(-c4ccnc(Nc5ccncc5F)n4)n3)no2)C1=O] (R)-3-(3-(6-(2-((3-Fluoropyridin- 4-yl)amino)pyrimidin-4- | ¹H NMR (400 MHz, DMSO-d₆) δ 9.88 (br s, 1H), 8.85-8.79 (m, 1H), 8.54-8.47 (m, 2H), 8.41-8.32 (m, 2H), 8.27-8.16 (m, 2H), 8.09-8.05 (m, 1H), 7.21 (s, 1H), 6.81 (s, 1H), 3.55-3.42 (m, 2H), 2.86 (s, 3H), 2.67- 2.59 (m, 1H), 2.35- 2.27 (m, 1H). MS (ESI): MS (ESI): Mass calcd. for C₂₂H₁₈FN₇O₃, 447.4; m/z found, 448.2 [M + H | RP HPLC (NH₄OH Modifier) | 28 |
| | yl)pyridin-2-yl)isoxazol-5-yl)-3- | | | |
| | hydroxy-1-methylpyrrolidin-2- | | | |
| | one | | | |
| 331 | 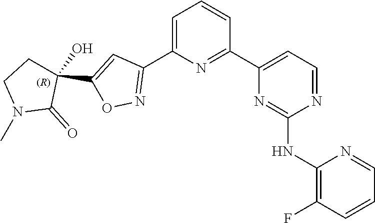 (R)-3-(3-(6-(2-((3-Fluoropyridin- 2-yl)amino)pyrimidin-4- yl)pyridin-2-yl)isoxazol-5-yl)-3- | ¹H NMR (400 MHz, DMSO-d₆) δ 10.01 (s, 1H), 8.68-8.65 (m, 1H), 8.30-8.23 (m, 2H), 8.17-8.12 (m, 2H), 7.94-7.90 (m, 1H), 7.83-7.76 (m, 1H), 7.36-7.30 (m, 1H), 7.18 (s, 1H), 6.79 (s, 1H), 3.52-3.42 (m, 2H), 2.85 (s, 3H), 2.66- 2.58 (m, 1H), 2.34- 2.26 (m, 1H). MS (ESI): Mass calcd. for C₂₂H₁₈FN₇O₃, 447.4; m/z found, 448.2 [M + H]⁺. | RP HPLC (NH₄OH Modifier) | 35 |
| | hydroxy-1-methylpyrrolidin-2- | | | |
| | one | | | |
| 332 | 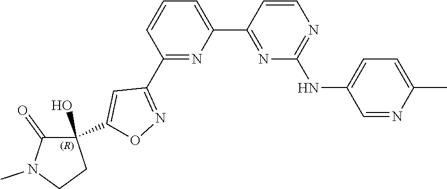 (R)-3-Hydroxy-1-methyl-3-(3- (6-(2-((6-methylpyridin-3- yl)amino)pyrimidin-4-yl)pyridin- 2-yl)isoxazol-5-yl)pyrrolidin-2-one | ¹H NMR (500 MHz, DMSO-d₆) δ 9.88 (s, 1H), 8.88 (d, J = 2.7 Hz, 1H), 8.72 (d, J = 5.0 Hz, 1H), 8.47 (dd, J = 7.7, 1.2 Hz, 1H), 8.23 (t, J = 7.7 Hz, 1H), 8.18 (dd, J = 7.8, 1.2 Hz, 1H), 8.15 (dd, J = 8.4, 2.7 Hz, 1H), 7.91 (d, J = 5.0 Hz, 1H), 7.25 (d, J = 8.4 Hz, 1H), 7.19 (s, 1H), 6.78 (s, 1H), 3.54-3.48 (m, 1H), 3.48-3.42 (m, 1H), 2.86 (s, 3H), 2.67- 2.60 (m, 1H), 2.45 (s, | RP HPLC (NH₄OH modifier) | 19 |
| | | 3H), 2.35-2.27 (m, | | |
| | | 1H). MS (ESI⁺): m/z = | | |
| | | 444.1. | | |
| 333 | 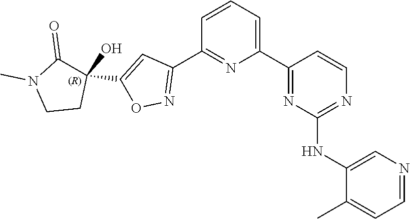 (R)-3-Hydroxy-1-methyl-3-(3- (6-(2-((4-methylpyridin-3- | ¹H NMR (500 MHz, DMSO-d₆) δ 9.64 (s, 1H), 9.23 (s, 1H), 8.75 (d, J = 5.0 Hz, 1H), 8.48 (d, J = 5.5 Hz, 1H), 8.37 (dd, J = 4.9, 4.0 Hz, 1H), 8.20- 8.16 (m, 2H), 8.00 (d, J = 5.0 Hz, 1H), 7.79 (d, J = 5.6 Hz, 1H), 7.19 (s, 1H), 3.54-3.48 (m, 3H), 3.48-3.42 (m, 2H), 2.86 (s, 3H), 2.66- 2.59 (m, 1H), 2.53 (s, 3H), 2.34-2.27 (m, 1H). 1H). MS (ESI⁺): m/z = | RP HPLC (TFA modifier) | 9 |
| | yl)amino)pyrimidin-4-yl)pyridin- | 444.1. | | |
| | 2-yl)isoxazol-5-yl)pyrrolidin-2- | | | |
| | one | | | |
| 334 | 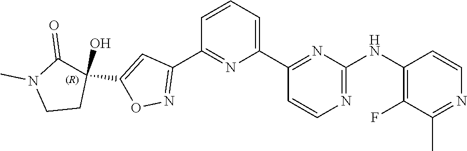 (R)-3-(3-(6-(2-((3-Fluoro-2- methylpyridin-4- yl)amino)pyrimidin-4-yl)pyridin- 2-yl)isoxazol-5-yl)-3-hydroxy-1- methylpyrrolidin-2-one | ¹H NMR (500 MHz, DMSO-d₆) δ 9.71 (s, 1H), 8.81 (d, J = 5.2 Hz, 1H), 8.52-8.48 (m, 1H), 8.25-8.20 (m, 3H), 8.20-8.18 (m, 1H), 8.07-8.04 (m, 1H), 7.20 (s, 1H), 6.78 (s, 1H), 3.54- 3.48 (m, 1H), 3.48- 3.43 (m, 1H), 2.86 (s, 3H), 2.67-2.60 (m, 1H), 2.46 (d, J = 3.3 Hz, 3H), 2.35-2.28 (m, 1H). MS (ESI⁺): | RP HPLC (NH₄OH modifier) | 29 |
| | | m/z = 462.1. | | |
| 335 | 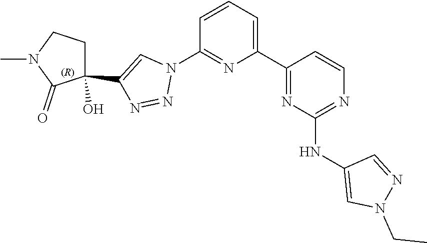 | ¹H NMR (400 MHz, DMSO-d₆) δ 9.66 (s, 1H), 8.94 (s, 1H), 8.68- 8.63 (m, 1H), 8.48 (s, 1H), 8.38-8.33 (m, 1H), 8.29-8.25 (m, 1H), 8.00 (s, 1H), 7.78- 7.74 (m, 1H), 7.62 (s, 1H), 4.20-4.09 (m, 2H), 2.83 (s, 3H), 2.81- 2.74 (m, 1H), 2.33- 2.23 (m, 1H), 1.47- 1.32 (m, 3H). MS (ESI⁺): m/z = 447.1. | RP HPLC (TFA modifier) | 30 |
| | (R)-3-(1-(6-(2-((1-Ethyl-1H- | | | |
| | pyrazol-4-yl)amino)pyrimidin-4- | | | |
| | yl)pyridin-2-yl)-1H-1,2,3-triazol- | | | |
| | 4-yl)-3-hydroxy-1- | | | |
| | methylpyrrolidin-2-one | | | |

The compounds shown in Table 5 were prepared in a manner analogous to (R)-3-hydroxy-1-methyl-3-(3-(6-(2-(oxetan-3-ylamino)pyrimidin-4-yl)pyridin-2-yl)isoxazol-5-yl)pyrrolidin-2-one (Example 66) using the appropriate amine in place of tert-butyl 3-amino-1H-pyrazole-1-carboxylate. Purification methods are noted in the table.

TABLE 5

| | Structure | | Purification | Yield |
|---|---|---|---|---|
| Ex. | Name | Analytical data | conditions | (%) |
| 336 and 337 | 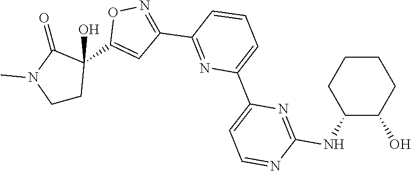 [SMILES: CN1CC[C@@](O)(c2cc(-c3cccc(-c4ccnc(N[C@@H]5CCCC[C@@H]5O)n4)n3)no2)C1=O] | Example 336: LCMS (ESI⁺): mass calcd. for C₂₃H₂₆N₆O₄ 450.2 m/z, found 451.7 [M + H]⁺; Example 337: LCMS (ESI⁺): mass calcd. For C₂₃H₂₆N₆O₄ 450.2 m/z, ound 451.7 [M + H]⁺ | RP HPLC (TFA modifier). The first eluting peak was designated Example 336 and the second eluting | Ex. 336: 11; |
| | | | peak was | |
| | 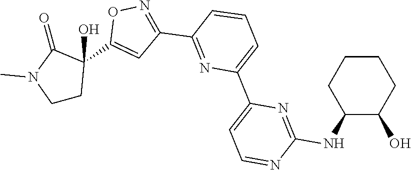 [SMILES: CN1CC[C@@](O)(c2cc(-c3cccc(-c4ccnc(N[C@H]5CCCC[C@H]5O)n4)n3)no2)C1=O] | | designated Example 337. | Ex. 337: 16 |
| | (R)-3-Hydroxy-3-(3-(6-(2-(((cis)-2- | | | |
| | hydroxycyclohexyl)amino) | | | |
| | pyrimidin-4-yl)pyridin-2-yl)isoxazol-5- | | | |
| | yl)-1-methylpyrrolidin-2-on and | | | |
| | (R)-3-hydroxy-3-(3-(6-(2- | | | |
| | (((trans)-2- | | | |
| | hydroxycyclohexyl)amino) | | | |
| | pyrimidin-4-yl)pyridin-2-yl)isoxazol-5- | | | |
| | yl)-1-methylpyrrolidin-2-one | | | |
| 338 | 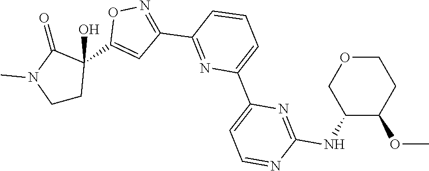 | LCMS (ESI⁺): mass calcd. for C₂₃H₂₆N₆O₅ 466.2 m/z, found 467.6 [M + H]⁺. | RP HPLC (NH₄OH modifier) | 21 |
| | (R)-3-Hydroxy-3-(3-(6-(2- | | | |
| | (((trans)-4-methoxytetrahydro- | | | |
| | 2H-pyran-3-yl)amino)pyrimidin- | | | |
| | 4-yl)pyridin-2-yl)isoxazol-5-yl)- | | | |
| | 1-methylpyrrolidin-2-one | | | |
| 339 | 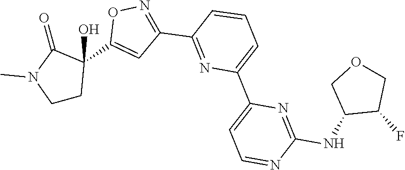 [SMILES: CN1CC[C@@](O)(c2cc(-c3cccc(-c4ccnc(N[C@@H]5COC[C@@H]5F)n4)n3)no2)C1=O] | LCMS (ESI⁺): mass calcd. for C₂₁H₂₁FN₆O₄ 440.2 m/z, found 441.6 [M + H]⁺. | RP HPLC (NH₄OH modifier) | 7 |
| | (R)-3-(3-(6-(2-(((cis)-4- | | | |
| | Fluorotetrahydrofuran-3- | | | |
| | yl)amino)pyrimidin-4-yl)pyridin- | | | |
| | 2-yl)isoxazol-5-yl)-3-hydroxy-1- | | | |
| | methylpyrrolidin-2-one | | | |
| 340 | 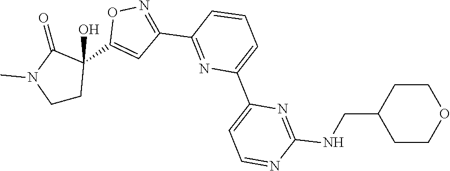 [SMILES: CN1CC[C@@](O)(c2cc(-c3cccc(-c4ccnc(NCC5CCOCC5)n4)n3)no2)C1=O] | LCMS (ESI⁺): mass calcd. for C₂₃H₂₆N₆O₄ 450.2 m/z, found 451.5 [M + H]⁺ | RP HPLC (TFA modifier) | 16 |
| | (R)-3-Hydroxy-1-methyl-3-(3- | | | |
| | (6-(2-(((tetrahydro-2H-pyran-4- | | | |
| | yl)methyl)amino)pyrimidin-4- | | | |
| | yl)pyridin-2-yl)isoxazol-5- | | | |
| | yl)pyrrolidin-2-one | | | |
| 341 | 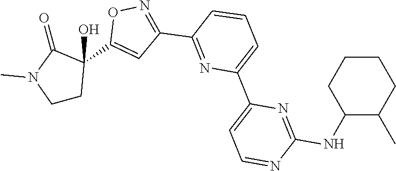 | LCMS (ESI⁺): mass calcd. for C₂₃H₂₆N₆O₄ 450.2 m/z, found 451.7 [M + H]⁺. | RP HPLC (TFA modifier) | 7 |
| | (3R)-3-Hydroxy-1-methyl-3-(3- | | | |
| | (6-(2-(((rac)-2- | | | |
| | methylcyclohexyl)amino) | | | |
| | pyrimidin-4-yl)pyridin-2-yl)isoxazol-5- | | | |
| | yl)pyrrolidin-2-one | | | |
| 342 | 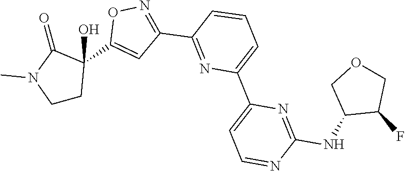 [SMILES: CN1CC[C@@](O)(c2cc(-c3cccc(-c4ccnc(N[C@@H]5COC[C@H]5F)n4)n3)no2)C1=O] | LCMS (ESI⁺): mass calcd. for C₂₁H₂₁FN₆O₄ 440.2 m/z, found 441.6 [M + H]⁺. | RP HPLC (NH₄OH modifier) | 7 |
| | (R)-3-(3-(6-(2-(((trans)-4- | | | |
| | Fluorotetrahydrofuran-3- | | | |
| | yl)amino)pyrimidin-4-yl)pyridin- | | | |
| | 2-yl)isoxazol-5-yl)-3-hydroxy-1- | | | |
| | methylpyrrolidin-2-one | | | |
| 343 | 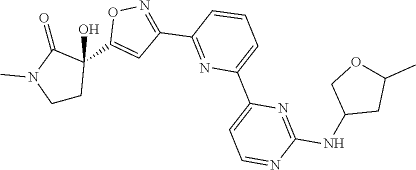 | LCMS (ESI⁺): mass calcd. for C₂₂H₂₄N₆O₄ 436.2 m/z, found 437.6 [M + H]⁺. | RP HPLC (TFA modifier) | 20 |
| | (R)-3-Hydroxy-1-methyl-3-(3- | | | |
| | (6-(2-(((rac)-5- | | | |
| | methyltetrahydrofuran-3- | | | |
| | yl)amino)pyrimidin-4-yl)pyridin- | | | |
| | 2-yl)isoxazol-5-yl)pyrrolidin-2- | | | |
| | one | | | |
| 344 | 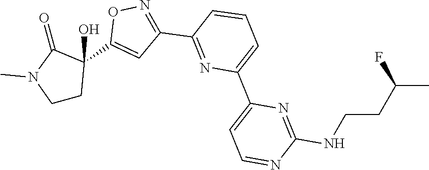 [SMILES: C[C@H](F)CCNc1nccc(-c2cccc(-c3cc([C@]4(O)CCN(C)C4=O)on3)n2)n1] | LCMS (ESI⁺): mass calcd. for C₂₁H₂₃FN₆O₃ 426.2 m/z, found 427.5 [M + H]⁺. | RP HPLC (TFA modifier) | 19 |
| | (R)-3-(3-(6-(2-(((S)-3- | | | |
| | Fluorobutyl)amino)pyrimidin-4- | | | |
| | yl)pyridin-2-yl)isoxazol-5-yl)-3- | | | |
| | hydroxy-1-methylpyrrolidin-2- | | | |
| | one | | | |
| 345 | 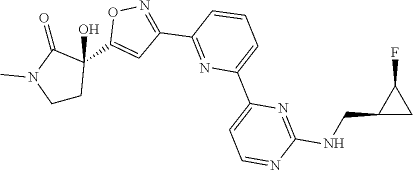 [SMILES: CN1CC[C@@](O)(c2cc(-c3cccc(-c4ccnc(NC[C@@H]5C[C@@H]5F)n4)n3)no2)C1=O] | LCMS (ESI⁺): mass calcd. for C₂₁H₂₁FN₆O₃ 424.2 m/z, found 425.5 [M + H]⁺. | RP HPLC (TFA modifier) | 19 |
| | (R)-3-(3-(6-(2-((((cis)-2- | | | |
| | Fluorocyclopropyl)methyl) | | | |
| | amino)pyrimidin-4-yl)pyridin-2- | | | |
| | yl)isoxazol-5-yl)-3-hydroxy-1- | | | |
| | methylpyrrolidin-2-one | | | |
| 346 and 347 | 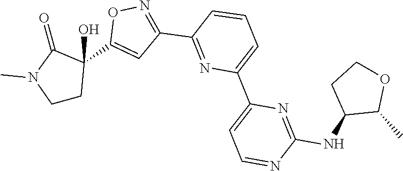 | Example 346: LCMS (ESI⁺): mass calcd. for C₂₂H₂₄N₆O₄ 436.2 m/z, found 437.5. [M + H]⁺. Example 347: LCMS (ESI⁺): mass calcd. for C₂₂H₂₄N₆O₄ 436.2 m/z, found 437.8. | RP HPLC (TFA modifier). The first eluting peak was designated Example 346 and the second eluting peak was designated Example 347 | Ex. 346: 11 Ex. 347: 12 |
| | 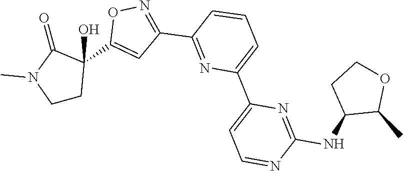 | | | |
| | (R)-3-Hydroxy-1-methyl-3-(3- | | | |
| | (6-(2-(((trans)-2- | | | |
| | methyltetrahydrofuran-3- | | | |
| | yl)amino)pyrimidin-4-yl)pyridin- | | | |
| | 2-yl)isoxazol-5-yl)pyrrolidin-2- | | | |
| | one; (R)-3-hydroxy-1-methyl-3- | | | |
| | (3-(6-(2-(((cis)-2- | | | |
| | methyltetrahydrofuran-3- | | | |
| | yl)amino)pyrimidin-4-yl)pyridin- | | | |
| | 2-yl)isoxazol-5-yl)pyrrolidin-2- | | | |
| | one | | | |
| 348 | 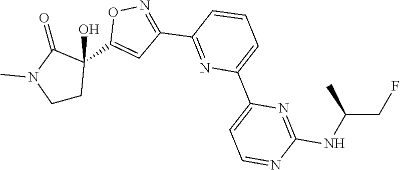 [SMILES: C[C@@H](CF)Nc1nccc(-c2cccc(-c3cc([C@]4(O)CCN(C)C4=O)on3)n2)n1] | LCMS (ESI⁺): mass calcd. for C₂₀H₂₁FN₆O₃ 412.2 m/z, found 413.4. | RP HPLC (TFA modifier) | 15 |
| | (R)-3-(3-(6-(2-(((S)-1- | | | |
| | Fluoropropan-2- | | | |
| | yl)amino)pyrimidin-4-yl)pyridin- | | | |
| | 2-yl)isoxazol-5-yl)-3-hydroxy-1- | | | |
| | methylpyrrolidin-2-one | | | |
| 349 | 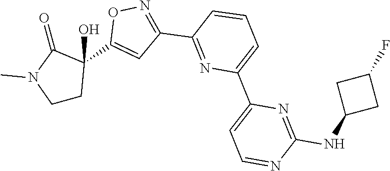 [SMILES: CN1CC[C@@](O)(c2cc(-c3cccc(-c4ccnc(N[C@H]5C[C@H](F)C5)n4)n3)no2)C1=O] | LCMS (ESI⁺): mass calcd. for C₂₁H₂₁FN₆O₃ 424.2 m/z, found 425.5. | RP HPLC (NH₄OH modifier) | 17 |
| | (R)-3-(3-(6-(2-(((1r,3R)-3- | | | |
| | Fluorocyclobutyl)amino)pyrimidin- | | | |
| | 4-yl)pyridin-2-yl)isoxazol-5- | | | |
| | yl)-3-hydroxy-1- | | | |
| | methylpyrrolidin-2-one | | | |
| 350 | 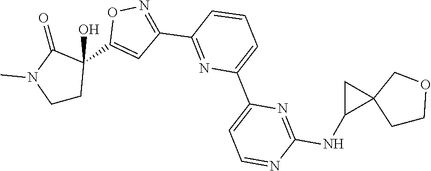 [SMILES: CN1CC[C@@](O)(c2cc(-c3cccc(-c4ccnc(NC5CC56CCOC6)n4)n3)no2)C1=O] | LCMS (ESI+): mass calcd. for C₂₃H₂₄N₆O₄ 448.2 m/z, found 449.4. | RP HPLC (TFA modifier) | 14 |
| | (3R)-3-(3-(6-(2-((5- | | | |
| | Oxaspiro[2.4]heptan-1- | | | |
| | yl)amino)pyrimidin-4-yl)pyridin- | | | |
| | 2-yl)isoxazol-5-yl)-3-hydroxy-1- | | | |
| | methylpyrrolidin-2-one | | | |
| 351 | 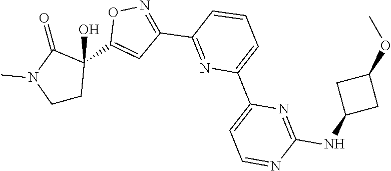 [SMILES: CO[C@H]1C[C@@H](Nc2nccc(-c3cccc(-c4cc([C@]5(O)CCN(C)C5=O)on4)n3)n2)C1] | LCMS (ESI⁺): mass calcd. for C₂₂H₂₄N₆O₄ 436.2 m/z, found 437.6. | RP HPLC (TFA modifier) | 19 |
| | (R)-3-Hydroxy-3-(3-(6-(2- | | | |
| | (((1s,3S)-3- | | | |
| | methoxycyclobutyl)amino) | | | |
| | pyrimidin-4-yl)pyridin-2-yl)isoxazol-5- | | | |
| | yl)-1-methylpyrrolidin-2-one | | | |
| 352 | 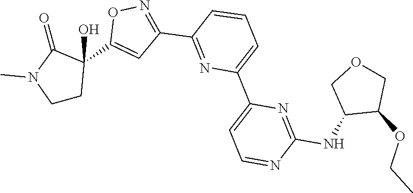 [SMILES: CCO[C@@H]1COC[C@H]1Nc1nccc(-c2cccc(-c3cc([C@]4(O)CCN(C)C4=O)on3)n2)n1] | LCMS (ESI⁺): mass calcd. for C₂₃H₂₆N₆O₅ 466.2 m/z, found 467.6. | RP HPLC (TFA modifier) | 13 |
| | (R)-3-(3-(6-(2-(((trans)-4- | | | |
| | Ethoxytetrahydrofuran-3- | | | |
| | yl)amino)pyrimidin-4-yl)pyridin- | | | |
| | 2-yl)isoxazol-5-yl)-3-hydroxy-1- | | | |
| | methylpyrrolidin-2-one | | | |
| 353 | 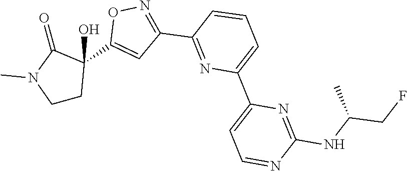 [SMILES: C[C@H](CF)Nc1nccc(-c2cccc(-c3cc([C@]4(O)CCN(C)C4=O)on3)n2)n1] | LCMS (ESI⁺): mass calcd. for C₂₀H₂₁FN₆O₃ 412.2 m/z, found 413.5. | RP HPLC (TFA modifier) | 10 |
| | (R)-3-(3-(6-(2-(((R)-1- | | | |
| | Fluoropropan-2- | | | |
| | yl)amino)pyrimidin-4-yl)pyridin- | | | |
| | 2-yl)isoxazol-5-yl)-3-hydroxy-1- | | | |
| | methylpyrrolidin-2-one | | | |
| 354 | 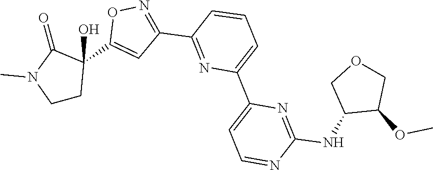 | LCMS (ESI⁺): mass calcd. for C₂₂H₂₄N₆O₅ 452.2 m/z, found 453.6. | RP HPLC (NH₄OH modifier) | 21 |
| | (R)-3-Hydroxy-3-(3-(6-(2- | | | |
| | (((3R,4S)-4- | | | |
| | methoxytetrahydrofuran-3- | | | |
| | yl)amino)pyrimidin-4-yl)pyridin- | | | |
| | 2-yl)isoxazol-5-yl)-1- | | | |
| | methylpyrrolidin-2-one | | | |
| 355 | 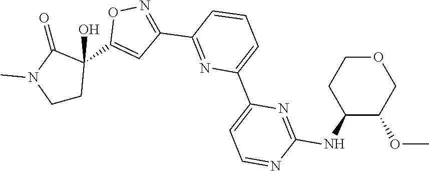 | LCMS (ESI⁺): mass calcd. for C₂₃H₂₆N₆O₅ 466.2 m/z, found 467.5. | RP HPLC (NH₄OH modifier) | 24 |
| | (R)-3-Hydroxy-3-(3-(6-(2- | | | |
| | (((trans)-3-methoxytetrahydro- | | | |
| | 2H-pyran-4-yl)amino)pyrimidin- | | | |
| | 4-yl)pyridin-2-yl)isoxazol-5-yl)- | | | |
| | 1-methylpyrrolidin-2-one | | | |
| 356 | 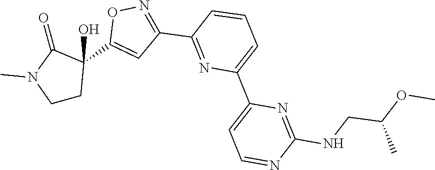 | LCMS (ESI⁺): mass calcd. for C₂₁H₂₄N₆O₄ 424.2 m/z, found 425.6. | RP HPLC (NH₄OH modifier) | 22 |
| | (R)-3-Hydroxy-3-(3-(6-(2-(((R)-2- | | | |
| | methoxypropyl)amino)pyrimidin- | | | |
| | 4-yl)pyridin-2-yl)isoxazol-5-yl)- | | | |
| | 1-methylpyrrolidin-2-one | | | |
| 357 | 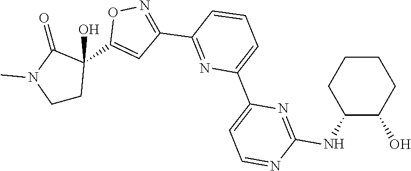 [SMILES: CN1CC[C@@](O)(c2cc(-c3cccc(-c4ccnc(N[C@@H]5CCCC[C@@H]5O)n4)n3)no2)C1=O] | LCMS (ESI⁺): mass calcd. for C₂₃H₂₆N₆O₄ 450.2 m/z, found 451.6. | RP HPLC (TFA modifier) | 14 |
| | (R)-3-Hydroxy-3-(3-(6-(2- | | | |
| | (((1R,2S)-2- | | | |
| | hydroxycyclohexyl)amino) | | | |
| | pyrimidin-4-yl)pyridin-2-yl)isoxazol-5- | | | |
| | yl)-1-methylpyrrolidin-2-one | | | |
| 358 | 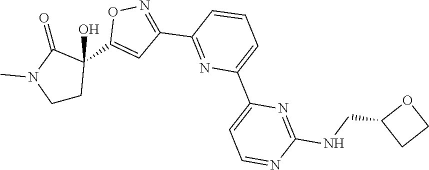 [SMILES: CN1CC[C@@](O)(c2cc(-c3cccc(-c4ccnc(NC[C@H]5CCO5)n4)n3)no2)C1=O] | LCMS (ESI⁺): mass calcd. for C₂₁H₂₂N₆O₄ 422.2 m/z, found 423.5. | RP HPLC (NH₄OH modifier) | 22 |
| | (R)-3-Hydroxy-1-methyl-3-(3- | | | |
| | (6-(2-((((R)-oxetan-2- | | | |
| | yl)methyl)amino)pyrimidin-4- | | | |
| | yl)pyridin-2-yl)isoxazol-5- | | | |
| | yl)pyrrolidin-2-one | | | |
| 359 | 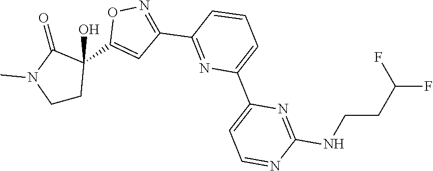 [SMILES: CN1CC[C@@](O)(c2cc(-c3cccc(-c4ccnc(NCCC(F)F)n4)n3)no2)C1=O] | LCMS (ESI⁺): mass calcd. for C₂₀H₂₀F₂N₆O₃ 430.2 m/z, found 431.5. | RP HPLC (TFA modifier) | 20 |
| | (R)-3-(3-(6-(2-((3,3- | | | |
| | Difluoropropyl)amino)pyrimidin- | | | |
| | 4-yl)pyridin-2-yl)isoxazol-5-yl)- | | | |
| | 3-hydroxy-1-methylpyrrolidin-2- | | | |
| | one | | | |
| 360 | 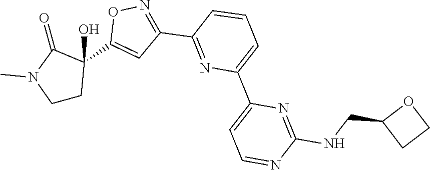 [SMILES: CN1CC[C@@](O)(c2cc(-c3cccc(-c4ccnc(NC[C@@H]5CCO5)n4)n3)no2)C1=O] | LCMS (ESI⁺): mass calcd. for C₂₁H₂₂N₆O₄ 422.2 m/z, found 423.5. | RP HPLC (NH₄OH modifier) | 25 |
| | (R)-3-Hydroxy-1-methyl-3-(3- | | | |
| | (6-(2-((((S)-oxetan-2- | | | |
| | yl)methyl)amino)pyrimidin-4- | | | |
| | yl)pyridin-2-yl)isoxazol-5- | | | |
| | yl)pyrrolidin-2-one | | | |
| 361 | 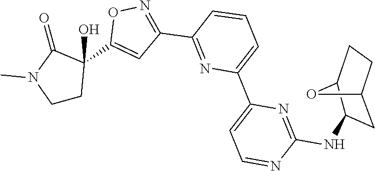 [SMILES: CN1CC[C@@](O)(c2cc(-c3cccc(-c4ccnc(N[C@@H]5CC6CCC5O6)n4)n3)no2)C1=O] | LCMS (ESI⁺): mass calcd. for C₂₃H₂₄N₆O₄ 448.2 m/z, found 449.5. | RP HPLC (TFA modifier) | 19 |
| | (3R)-3-(3-(6-(2-(((cis)-7- | | | |
| | Oxabicyclo[2.2.1]heptan-2- | | | |
| | yl)amino)pyrimidin-4-yl)pyridin- | | | |
| | 2-yl)isoxazol-5-yl)-3-hydroxy-1- | | | |
| | methylpyrrolidin-2-one | | | |
| 362 | 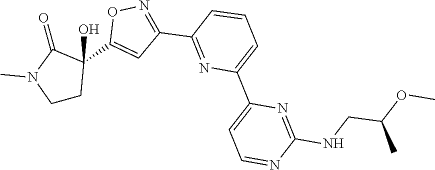 | LCMS (ESI⁺): mass calcd. for C₂₁H₂₄N₆O₄ 424.2 m/z, found 425.5. | RP HPLC (TFA modifier) | 20 |
| | (R)-3-Hydroxy-3-(3-(6-(2-(((S)-2- | | | |
| | methoxypropyl)amino)pyrimidin- | | | |
| | 4-yl)pyridin-2-yl)isoxazol-5-yl)- | | | |
| | 1-methylpyrrolidin-2-one | | | |
| 363 | 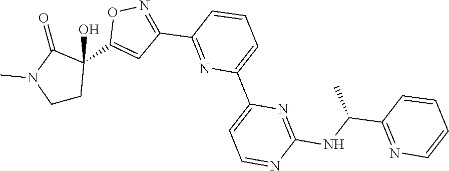 [SMILES: C[C@@H](Nc1nccc(-c2cccc(-c3cc([C@]4(O)CCN(C)C4=O)on3)n2)n1)c1ccccn1] | LCMS (ESI⁺): mass calcd. for C₂₄H₂₃N₇O₃ 457.2 m/z, found 458.5. | RP HPLC (TFA modifier) | 17 |
| | (R)-3-Hydroxy-1-methyl-3-(3- | | | |
| | (6-(2-(((R)-1-(pyridin-2- | | | |
| | yl)ethyl)amino)pyrimidin-4- | | | |
| | yl)pyridin-2-yl)isoxazol-5- | | | |
| | yl)pyrrolidin-2-one | | | |
| 364 | 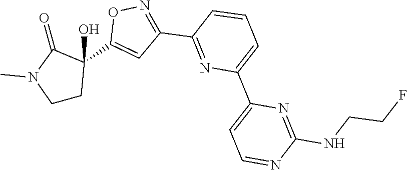 [SMILES: CN1CC[C@@](O)(c2cc(-c3cccc(-c4ccnc(NCCF)n4)n3)no2)C1=O] | LCMS (ESI⁺): mass calcd. for C₁₉H₁₉FN₆O₃ 398.2 m/z, found 399.5. | RP HPLC (TFA modifier) | 15 |
| | (R)-3-(3-(6-(2-((2- | | | |
| | Fluoroethyl)amino)pyrimidin-4- | | | |
| | yl)pyridin-2-yl)isoxazol-5-yl)-3- | | | |
| | hydroxy-1-methylpyrrolidin-2- | | | |
| | one | | | |
| 365 | 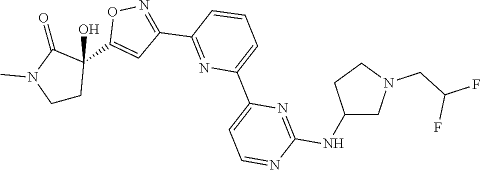 [SMILES: CN1CC[C@@](O)(c2cc(-c3cccc(-c4ccnc(NC5CCN(CC(F)F)C5)n4)n3)no2)C1=O] | LCMS (ESI⁺): mass calcd. for C₂₃H₂₅F₂N₇O₃ 485.2 m/z, found 486.6. | RP HPLC (TFA modifier) | 19 |
| | (3R)-3-(3-(6-(2-((1-(2,2- | | | |
| | Difluoroethyl)pyrrolidin-3- | | | |
| | yl)amino)pyrimidin-4-yl)pyridin- | | | |
| | 2-yl)isoxazol-5-yl)-3-hydroxy-1- | | | |
| | methylpyrrolidin-2-one | | | |
| 366 | 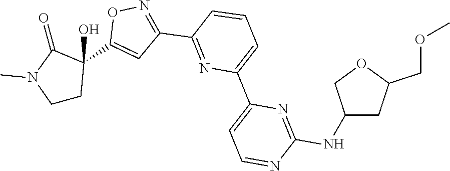 | LCMS (ESI⁺): mass calcd. for C₂₃H₂₆N₆O₅ 466.2 m/z, found 467.6. | RP HPLC (NH₄OH modifier) | 9 |
| | (3R)-3-Hydroxy-3-(3-(6-(2-((5- | | | |
| | (methoxymethyl)tetrahydrofuran- | | | |
| | 3-yl)amino)pyrimidin-4- | | | |
| | yl)pyridin-2-yl)isoxazol-5-yl)-1- | | | |
| | methylpyrrolidin-2-one | | | |
| 367 | 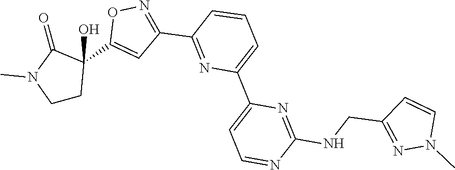 | ¹H NMR (401 MHz, DMSO-d₆) δ 8.52 (d, J = 5.2 Hz, 1H), 8.48 (s, 1H), 8.23-8.08 (m, 2H), 7.69 (d, J = 5.2 Hz, 1H), 7.57 (d, J = 2.2 Hz, 1H), 7.16 (s, 1H), 6.18 (d, J = 2.2 Hz, 1H), 4.58 (s, 2H), 3.79 (s, 3H), 3.48 (dddd, J = 17.5, 10.0, | RP HPLC (TFA modifier) | 10 |
| | | 7.7, 5.2 Hz, 2H), 2.86 | | |
| | (R)-3-Hydroxy-1-methyl-3-(3- | (s, 3H), 2.71-2.58 (m, | | |
| | (6-(2-(((1-methyl-1H-pyrazol-3- | 1H), 2.38-2.24 (m, | | |
| | yl)methyl)amino)pyrimidin-4- | 1H). LCMS (ESI⁺): | | |
| | yl)pyridin-2-yl)isoxazol-5- | m/z = 447.3. | | |
| | yl)pyrrolidin-2-one | | | |
| 368 | 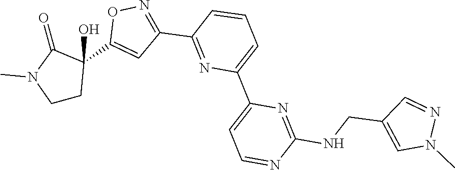 | ¹H NMR (401 MHz, DMSO-d₆) δ 8.55- 8.43 (m, 1H), 8.23- 8.11 (m, 2H), 7.69- 7.53 (m, 3H), 7.41 (s, 1H), 7.15 (s, 1H), 4.51- 4.34 (m, 2H), 3.77 (s, 4H), 3.60-3.39 (m, 2H, overlapping with residual water signal), 2.86 (s, 3H), 2.71- | RP HPLC (NH₄OH modifier) | 14 |
| | | 2.55 (m, 1H), 2.44- | | |
| | (R)-3-Hydroxy-1-methyl-3-(3- | 2.23 (m, 1H). LCMS | | |
| | (6-(2-(((1-methyl-1H-pyrazol-4- | (ESI+): m/z = 447.2. | | |
| | yl)methyl)amino)pyrimidin-4- | | | |
| | yl)pyridin-2-yl)isoxazol-5- | | | |
| | yl)pyrrolidin-2-one | | | |
| 369 | 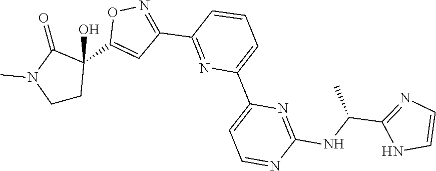 [SMILES: C[C@@H](Nc1nccc(-c2cccc(-c3cc([C@]4(O)CCN(C)C4=O)on3)n2)n1)c1ncc[nH]1] | ¹H NMR (401 MHz, DMSO-d₆) δ 8.54 (d, J = 5.0 Hz, 1H), 8.41 (d, J = 6.7 Hz, 1H), 8.14 (d, J = 2.8 Hz, 2H), 7.70 (d, J = 5.1 Hz, 1H), 7.62 (d, J = 7.4 Hz, 1H), 7.15 (s, 1H), 7.04 (s, 2H), 6.82 (s, 1H), 5.45-5.22 (m, 1H), 3.73-3.31 (m, | RP HPLC (NH4OH modifier) | 15 |
| | | 2H, overlapping with | | |
| | (R)-3-(3-(6-(2-(((R)-1-(1H- | residual water signal), | | |
| | Imidazol-2- | 3.18 (s, 1H), 2.86 (s, | | |
| | yl)ethyl)amino)pyrimidin-4- | 4H), 2.70-2.59 (m, | | |
| | yl)pyridin-2-yl)isoxazol-5-yl)-3- | 1H), 2.35-2.21 (m, | | |
| | hydroxy-1-methylpyrrolidin-2- | 1H), 1.59 (d, J = 6.9 | | |
| | one | Hz, 3H). LCMS (ESI⁺): | | |
| | | m/z = 447.2. | | |
| 370 | 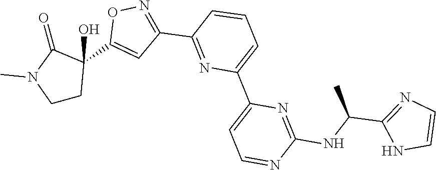 [SMILES: C[C@H](Nc1nccc(-c2cccc(-c3cc([C@]4(O)CCN(C)C4=O)on3)n2)n1)c1ncc[nH]1] | ¹H NMR (401 MHz, DMSO-d₆) δ 8.54 (d, J = 5.0 Hz, 1H), 8.40 (d, J = 6.7 Hz, 1H), 8.13 (q, J = 4.8, 4.2 Hz, 2H), 7.69 (d, J = 5.0 Hz, 1H), 7.62 (d, J = 7.3 Hz, 1H), 7.15 (s, 1H), 7.05 (s, 2H), 6.93- 6.64 (m, 1H), 5.45- 5.21 (m, 1H), 3.61- | RP HPLC (NH₄OH modifier) | 15 |
| | | 3.35 (m, 2H, | | |
| | (R)-3-(3-(6-(2-(((S)-1-(1H- | overlapping with | | |
| | Imidazol-2- | residual water signal), | | |
| | yl)ethyl)amino)pyrimidin-4- | 2.85 (s, 4H), 2.69- | | |
| | yl)pyridin-2-yl)isoxazol-5-yl)-3- | 2.57 (m, 1H), 2.38- | | |
| | hydroxy-1-methylpyrrolidin-2- | 2.20 (m, 1H), 1.58 (d, | | |
| | one | J = 6.9 Hz, 3H). LCMS | | |
| | | (ESI⁺): m/z = 447.3. | | |
| 371 | 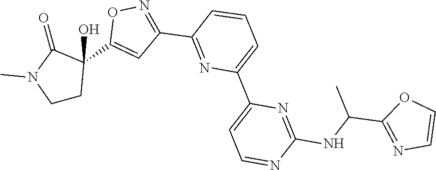 [SMILES: CC(Nc1nccc(-c2cccc(-c3cc([C@]4(O)CCN(C)C4=O)on3)n2)n1)c1ncco1] | ¹H NMR (401 MHz, DMSO-d₆) δ 8.52 (d, J = 5.0 Hz, 1H), 8.38 (d, J = 8.2 Hz, 1H), 8.20- 8.08 (m, 2H), 7.97 (d, J = 18.1 Hz, 2H), 7.93 (s, 1H), 7.68 (d, J = 5.0 Hz, 1H), 7.14 (d, J = 5.0 Hz, 2H), 6.80 (s, 1H), 5.35 (p, J = 7.1 Hz, 1H), 3.65-3.30 | RP HPLC (NH₄OH modifier) | 4 |
| | | (m, 2H, overlapping | | |
| | (3R)-3-Hydroxy-1-methyl-3-(3- | with residual water | | |
| | (6-(2-((1-(oxazol-2- | signal), 2.85 (s, 3H), | | |
| | yl)ethyl)amino)pyrimidin-4- | 2.67-2.56 (m, 1H), | | |
| | yl)pyridin-2-yl)isoxazol-5- | 2.35-2.21 (m, 1H), | | |
| | yl)pyrrolidin-2-one | 1.60 (d, J = 7.1 Hz, | | |
| | | 3H). LCMS (ESI⁺): | | |
| | | m/z = 447.2. | | |
| 372 | 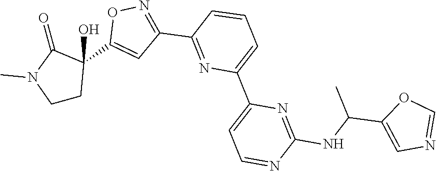 [SMILES: CC(Nc1nccc(-c2cccc(-c3cc([C@]4(O)CCN(C)C4=O)on3)n2)n1)c1cnco1] | ¹H NMR (401 MHz, DMSO-d₆) δ 8.54 (d, J = 5.0 Hz, 1H), 8.46 (d, J = 7.1 Hz, 1H), 8.25 (s, 1H), 8.20-8.04 (m, 2H), 7.83 (d, J = 8.2 Hz, 1H), 7.69 (d, J = 5.0 Hz, 1H), 7.15 (s, 1H), 6.81 (s, 1H), 5.45 (s, 1H), 3.72-3.30 (m, 2H, overlapping with | RP HPLC (NH₄OH modifier) | 3 |
| | | residual water signal), | | |
| | (3R)-3-Hydroxy-1-methyl-3-(3- | 2.85 (s, 3H), 2.67- | | |
| | (6-(2-((1-(oxazol-5- | 2.56 (m, 1H), 2.38- | | |
| | yl)ethyl)amino)pyrimidin-4- | 2.20 (m, 1H), 1.56 (d, | | |
| | yl)pyridin-2-yl)isoxazol-5- | J = 7.0 Hz, 3H). LCMS | | |
| | yl)pyrrolidin-2-one | (ESI⁺): m/z = 447.2. | | |
| 373 | 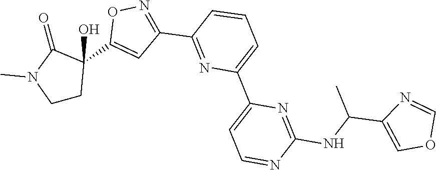 [SMILES: CC(Nc1nccc(-c2cccc(-c3cc([C@]4(O)CCN(C)C4=O)on3)n2)n1)c1cocn1] | ¹H NMR (401 MHz, DMSO-d₆) δ 8.52 (d, J = 5.0 Hz, 1H), 8.45 (dd, J = 7.1, 1.9 Hz, 1H), 8.29 (s, 1H), 8.21- 8.09 (m, 2H), 7.95 (s, 1H), 7.67 (d, J = 5.0 Hz, 1H), 7.61 (d, J = 8.2 Hz, 1H), 7.15 (s, 1H), 5.27 (s, 1H), 3.66- 3.41 (m, 2H, | RP HPLC (TFA modifier) | 12 |
| | | overlapping with | | |
| | (3R)-3-Hydroxy-1-methyl-3-(3- | residual water signal), | | |
| | (6-(2-((1-(oxazol-4- | 2.85 (s, 3H), 2.70- | | |
| | yl)ethyl)amino)pyrimidin-4- | 2.55 (m, 2H), 2.38- | | |
| | yl)pyridin-2-yl)isoxazol-5- | 2.18 (m, 1H), 1.52 (d, | | |
| | yl)pyrrolidin-2-one | J = 6.9 Hz, 3H). LCMS | | |
| | | (ESI⁺): m/z = 448.2. | | |
| 374 | 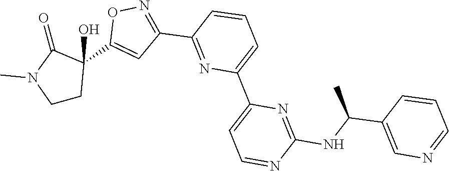 [SMILES: C[C@H](Nc1nccc(-c2cccc(-c3cc([C@]4(O)CCN(C)C4=O)on3)n2)n1)c1cccnc1] | ¹H NMR (401 MHz, DMSO-d₆) δ 8.71 (s, 1H), 8.48 (d, J = 5.0 Hz, 1H), 8.46-8.29 (m, 1H), 8.23-8.09 (m, 2H), 7.98 (d, J = 7.8 Hz, 1H), 7.90 (d, J = 7.8 Hz, 1H), 7.63 (d, J = 5.0 Hz, 1H), 7.37 (dd, J = 7.9, 4.8 Hz, 1H), 7.13 (s, 1H), 6.81 | RP HPLC (NH₄OH modifier) | 7 |
| | | (s, 1H), 5.25 (s, 1H), | | |
| | (R)-3-Hydroxy-1-methyl-3-(3- | 3.66-3.32 (m, 2H, | | |
| | (6-(2-(((S)-1-(pyridin-3- | overlapping with | | |
| | yl)ethyl)amino)pyrimidin-4- | residual water signal), | | |
| | yl)pyridin-2-yl)isoxazol-5- | 2.85 (s, 3H), 2.68- | | |
| | yl)pyrrolidin-2-one | 2.55 (m, 1H), 2.39- | | |
| | | 2.19 (m, 1H), 1.54 (d, | | |
| | | J = 7.1 Hz, 3H). LCMS | | |
| | | (ESI⁺): m/z = 458.3. | | |
| 375 | 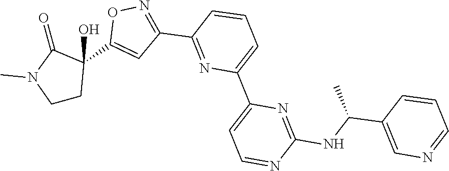 [SMILES: C[C@@H](Nc1nccc(-c2cccc(-c3cc([C@]4(O)CCN(C)C4=O)on3)n2)n1)c1cccnc1] | ¹H NMR (401 MHz, DMSO-d₆) δ 8.71 (s, 1H), 8.48 (d, J = 5.0 Hz, 1H), 8.41 (dd, J = 4.8, 1.6 Hz, 1H), 8.24- 8.04 (m, 2H), 7.97 (d, J = 7.8 Hz, 1H), 7.90 (d, J = 7.9 Hz, 1H), 7.62 (d, J = 5.0 Hz, 1H), 7.38 (dd, J = 7.9, 4.8 Hz, 1H), 7.12 (s, 1H), | RP HPLC (NH₄OH modifier) | 12 |
| | | 6.83 (s, 1H), 5.25 (s, | | |
| | (R)-3-Hydroxy-1-methyl-3-(3- | 1H), 3.83-3.23 (m, | | |
| | (6-(2-(((R)-1-(pyridin-3- | 2H), 2.84 (s, 3H), 2.66- | | |
| | yl)ethyl)amino)pyrimidin-4- | 2.54 (m, 1H), 2.38- | | |
| | yl)pyridin-2-yl)isoxazol-5- | 2.18 (m, 1H), 1.54 (d, | | |
| | yl)pyrrolidin-2-one | J = 7.1 Hz, 3H). LCMS | | |
| | | (ESI⁺): m/z = 458.1. | | |
| 376 | 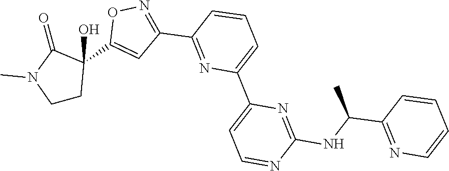 [SMILES: C[C@H](Nc1nccc(-c2cccc(-c3cc([C@]4(O)CCN(C)C4=O)on3)n2)n1)c1ccccn1] | ¹H NMR (401 MHz, DMSO-d₆) δ 8.69 (d, J = 5.3 Hz, 1H), 8.51 (s, 1H), 8.17 (t, J = 8.0 Hz, 1H), 8.11 (s, 4H), 7.82 (d, J = 8.1 Hz, 1H), 7.68 (d, J = 5.0 Hz, 1H), 7.59 (dd, J = 7.5, 5.4 Hz, 1H), 7.12 (s, 1H), 5.33 (s, 1H), 3.54- 3.38 (m, 2H), 2.85 (s, | RP HPLC (TFA modifier) | 12 |
| | | 3H), 2.65-2.57 (m, | | |
| | (R)-3-Hydroxy-1-methyl-3-(3- | 1H), 2.39-2.21 (m, | | |
| | (6-(2-(((S)-1-(pyridin-2- | 1H), 1.60 (d, J = 7.0 | | |
| | yl)ethyl)amino)pyrimidin-4- | Hz, 3H). LCMS (ESI⁺): | | |
| | yl)pyridin-2-yl)isoxazol-5- | m/z = 458.5. | | |
| | yl)pyrrolidin-2-one | | | |
| 377 | 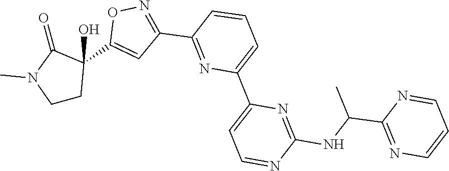 [SMILES: CC(Nc1nccc(-c2cccc(-c3cc([C@]4(O)CCN(C)C4=O)on3)n2)n1)c1ncccn1] | ¹H NMR (401 MHz, DMSO-d₆) δ 8.78 (d, J = 4.9 Hz, 2H), 8.47 (s, 1H), 8.10 (s, 3H), 7.69 (s, 1H), 7.62 (d, J = 5.0 Hz, 1H), 7.35 (t, J = 4.9 Hz, 1H), 7.13 (s, 1H), 5.24 (p, J = 7.0 Hz, 1H), 3.75-3.32 (m, 2H), 2.85 (s, 3H), 2.68- 2.54 (m, 1H), 2.37- | RP HPLC (NH₄OH modifier) | 15 |
| | | 2.22 (m, 1H), 1.58 (d, | | |
| | (3R)-3-Hydroxy-1-methyl-3-(3- | J = 7.1 Hz, 3H). LCMS | | |
| | (6-(2-((1-(pyrimidin-2- | (ESI⁺): m/z = 459.2. | | |
| | yl)ethyl)amino)pyrimidin-4- | | | |
| | yl)pyridin-2-yl)isoxazol-5- | | | |
| | yl)pyrrolidin-2-one | | | |
| 378 | 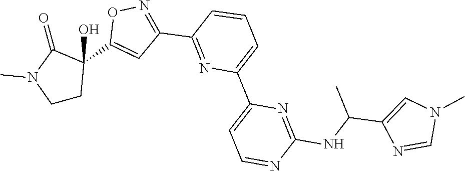 | ¹H NMR (401 MHz, DMSO-d₆) δ 8.51 (d, J = 5.0 Hz, 1H), 8.46 (dd, J = 7.1, 1.8 Hz, 1H), 8.23-8.08 (m, 2H), 7.70-7.57 (m, 2H), 7.33 (d, J = 8.3 Hz, 1H), 7.15 (s, 1H), 7.04 (s, 1H), 6.83 (s, 1H), 5.24 (s, 1H), 3.62 (s, 3H), 3.51-3.36 (m, | RP HPLC (NH₄OH modifier) | 15 |
| | | 2H), 2.86 (s, 3H), 2.72- | | |
| | (3R)-3-Hydroxy-1-methyl-3-(3- | 2.56 (m, 1H), 2.36- | | |
| | (6-(2-((1-(1-methyl-1H- | 2.17 (m, 1H), 1.50 (d, | | |
| | imidazol-4- | J = 6.8 Hz, 3H). LCMS | | |
| | yl)ethyl)amino)pyrimidin-4- | (ESI⁺): m/z = 461.3. | | |
| | yl)pyridin-2-yl)isoxazol-5- | | | |
| | yl)pyrrolidin-2-one | | | |
| 379 | 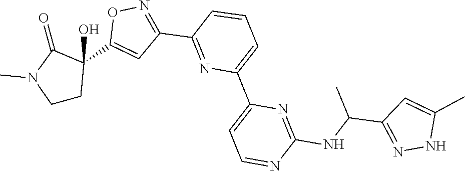 | ¹H NMR (401 MHz, DMSO-d₆) δ 12.50- 11.75 (m, 1H), 8.48 (dd, J = 15.3, 6.1 Hz, 2H), 8.24-8.01 (m, 2H), 7.63 (d, J = 5.0 Hz, 1H), 7.44 (s, 1H), 7.15 (s, 1H), 6.82 (s, 1H), 5.97 (s, 1H), 5.30 (s, 1H), 3.59-3.40 (m, 2H), 2.86 (s, 3H), 2.68- | RP HPLC (NH₄OH modifier) | 15 |
| | | 2.53 (m, 1H), 2.36- | | |
| | (3R)-3-Hydroxy-1-methyl-3-(3- | 2.23 (m, 1H), 2.16 (s, | | |
| | (6-(2-((1-(5-methyl-1H-pyrazol- | 3H), 1.50 (d, J = 7.0 | | |
| | 3-yl)ethyl)amino)pyrimidin-4- | Hz, 3H). LCMS (ESI⁺): | | |
| | yl)pyridin-2-yl)isoxazol-5- | m/z = 461.3. | | |
| | yl)pyrrolidin-2-one | | | |
| 380 | 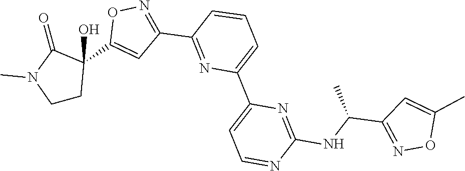 | ¹H NMR (401 MHz, DMSO-d₆) δ 8.53 (d, J = 5.0 Hz, 1H), 8.44 (d, J = 7.2 Hz, 1H), 8.23- 8.06 (m, 2H), 7.85 (s, 1H), 7.68 (d, J = 5.1 Hz, 1H), 7.15 (s, 1H), 6.21 (s, 1H), 5.37 (s, 1H), 3.55-3.35 (m, 2H), 2.85 (s, 3H), 2.72- 2.52 (m, 1H), 2.34 (s, | RP HPLC (TFA modifier) | 12 |
| | | 3H), 2.33-2.25 (m, | | |
| | (R)-3-Hydroxy-1-methyl-3-(3- | 1H), 1.53 (d, J = 7.0 | | |
| | (6-(2-(((R)-1-(5-methylisoxazol- | Hz, 3H). LCMS (ESI⁺): | | |
| | 3-yl)ethyl)amino)pyrimidin-4- | m/z = 462.0. | | |
| | yl)pyridin-2-yl)isoxazol-5- | | | |
| | yl)pyrrolidin-2-one | | | |
| 381 | 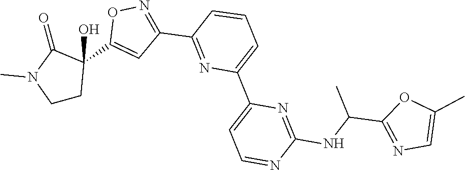 | ¹H NMR (401 MHz, DMSO-d₆) δ 8.52 (d, J = 5.0 Hz, 1H), 8.41 (d, J = 7.2 Hz, 1H), 8.20- 8.08 (m, 2H), 7.86 (d, J = 7.6 Hz, 1H), 7.68 (d, J = 5.0 Hz, 1H), 7.15 (s, 1H), 6.82 (s, 1H), 6.72 (d, J = 1.4 Hz, 1H), 5.29 (p, J = 7.1 Hz, 1H), 3.67-3.33 | RP HPLC (NH₄OH modifier) | 9 |
| | | (m, 2H), 2.85 (s, 3H), | | |
| | (3R)-3-Hydroxy-1-methyl-3-(3- | 2.62 (ddd, J = 13.0, | | |
| | (6-(2-((1-(5-methyloxazol-2- | 7.6, 5.0 Hz, 1H), 2.30 | | |
| | yl)ethyl)amino)pyrimidin-4- | (ddd, J = 13.5, 8.0, 5.6 | | |
| | yl)pyridin-2-yl)isoxazol-5- | Hz, 1H), 2.25 (d, J = | | |
| | yl)pyrrolidin-2-one | 1.3 Hz, 3H), 1.57 (d, | | |
| | | J = 7.1 Hz, 3H). LCMS | | |
| | | (ESI⁺): m/z = 462.3. | | |
| 382 | 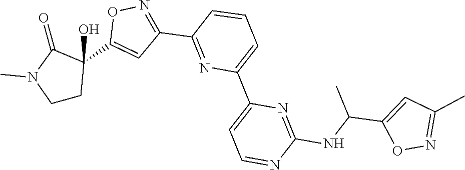 | ¹H NMR (401 MHz, DMSO-d₆) δ 8.54 (d, J = 5.0 Hz, 1H), 8.43 (dd, J = 7.1, 1.8 Hz, 1H), 8.21-8.06 (m, 2H), 7.95 (d, J = 8.1 Hz, 1H), 7.70 (d, J = 5.0 Hz, 1H), 7.15 (s, 1H), 6.21 (s, 1H), 5.43 (s, 1H), 3.50-3.40 (m, 2H), 2.85 (s, 3H), 2.71- | RP HPLC (TFA modifier) | 11 |
| | | 2.58 (m, 1H), 2.39- | | |
| | (3R)-3-Hydroxy-1-methyl-3-(3- | 2.25 (m, 1H), 2.17 (s, | | |
| | (6-(2-((1-(3-methylisoxazol-5- | 3H), 1.56 (d, J = 7.1 | | |
| | yl)ethyl)amino)pyrimidin-4- | Hz, 3H). LCMS (ESI⁺): | | |
| | yl)pyridin-2-yl)isoxazol-5- | m/z = 462.3. | | |
| | yl)pyrrolidin-2-one | | | |
| 383 | 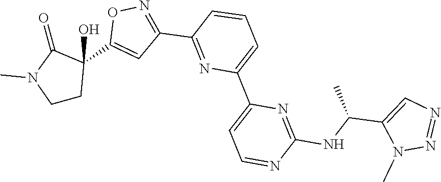 [SMILES: C[C@@H](Nc1nccc(-c2cccc(-c3cc([C@]4(O)CCN(C)C4=O)on3)n2)n1)c1cnnn1C] | ¹H NMR (401 MHz, DMSO-d₆) δ 8.54 (d, J = 5.0 Hz, 1H), 8.43 (dd, J = 7.2, 1.7 Hz, 1H), 8.24-8.08 (m, 2H), 7.90 (d, J = 8.0 Hz, 1H), 7.75-7.63 (m, 2H), 7.14 (s, 1H), 6.83 (s, 1H), 5.49 (s, 1H), 4.05 (s, 3H), 3.54- 3.35 (m, 2H), 2.85 (s, | RP HPLC (NH₄OH modifier) | 12 |
| | | 3H), 2.69-2.57 (m, | | |
| | (R)-3-Hydroxy-1-methyl-3-(3- | 1H), 2.37-2.19 (m, | | |
| | (6-(2-(((R)-1-(1-methyl-1H- | 1H), 1.58 (d, J = 7.0 | | |
| | 1,2,3-triazol-5- | Hz, 3H). LCMS (ESI⁺): | | |
| | yl)ethyl)amino)pyrimidin-4- | m/z = 462.6. | | |
| | yl)pyridin-2-yl)isoxazol-5- | | | |
| | yl)pyrrolidin-2-one | | | |
| 384 | 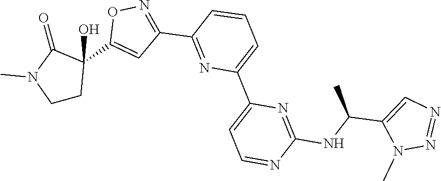 [SMILES: C[C@H](Nc1nccc(-c2cccc(-c3cc([C@]4(O)CCN(C)C4=O)on3)n2)n1)c1cnnn1C] | ¹H NMR (401 MHz, DMSO-d₆) δ 8.54 (d, J = 5.0 Hz, 1H), 8.43 (dd, J = 7.2, 1.7 Hz, 1H), 8.20-8.09 (m, 2H), 7.91 (d, J = 8.1 Hz, 1H), 7.75-7.63 (m, 2H), 7.14 (s, 1H), 6.82 (s, 1H), 5.49 (s, 1H), 4.05 (s, 3H), 3.49- 3.36 (m, 2H), 2.85 (s, | RP HPLC (NH₄OH modifier) | 10 |
| | | 3H), 2.67-2.56 (m, | | |
| | (R)-3-Hydroxy-1-methyl-3-(3- | 1H), 2.39-2.21 (m, | | |
| | (6-(2-(((S)-1-(1-methyl-1H- | 1H), 1.58 (d, J = 6.9 | | |
| | 1,2,3-triazol-5- | Hz, 3H). LCMS (ESI⁺): | | |
| | yl)ethyl)amino)pyrimidin-4- | m/z = 462.3. | | |
| | yl)pyridin-2-yl)isoxazol-5- | | | |
| | yl)pyrrolidin-2-one | | | |
| 385 | 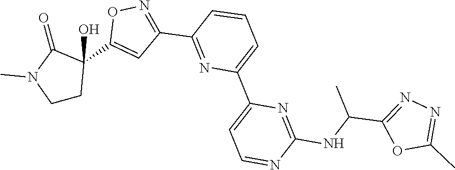 | ¹H NMR (401 MHz, DMSO-d₆) δ 8.55 (d, J = 5.0 Hz, 1H), 8.40 (d, J = 7.2 Hz, 1H), 8.21- 8.07 (m, 2H), 8.05 (d, J = 7.4 Hz, 1H), 7.72 (d, J = 5.0 Hz, 1H), 7.15 (s, 1H), 6.80 (s, 1H), 5.45 (p, J = 7.1 Hz, 1H), 3.56-3.28 (m, 2H), 2.86 (s, 3H), 2.71- | RP HPLC (NH₄OH modifier) | 5 |
| | | 2.58 (m, 1H), 2.46 (s, | | |
| | (3R)-3-Hydroxy-1-methyl-3-(3- | 3H), 2.35-2.16 (m, | | |
| | (6-(2-((1-(5-methyl-1,3,4- | 1H), 1.64 (d, J = 7.1 | | |
| | oxadiazol-2- | Hz, 3H). LCMS (ESI⁺): | | |
| | yl)ethyl)amino)pyrimidin-4- | m/z = 463.1. | | |
| | yl)pyridin-2-yl)isoxazol-5- | | | |
| | yl)pyrrolidin-2-one | | | |
| 386 | 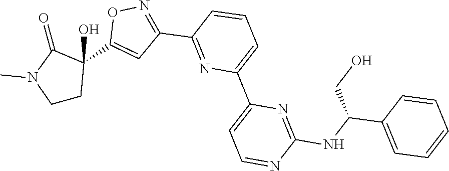 [SMILES: CN1CC[C@@](O)(c2cc(-c3cccc(-c4ccnc(N[C@H](CO)c5ccccc5)n4)n3)no2)C1=O] | ¹H NMR (401 MHz, DMSO-d₆) δ 8.53- 8.44 (m, 1H), 8.38 (s, 2H), 8.20-8.07 (m, 2H), 7.72-7.64 (m, 1H), 7.62 (d, J = 5.0 Hz, 1H), 7.56-7.40 (m, 2H), 7.32 (t, J = 7.5 Hz, 2H), 7.21 (t, J = 7.3 Hz, 1H), 7.13 (s, 1H), 6.80 (s, 1H), 5.20- | RP HPLC (NH₄OH modifier) | 9 |
| | | 5.07 (m, 1H), 5.03- | | |
| | (R)-3-Hydroxy-3-(3-(6-(2-(((S)- | 4.91 (m, 1H), 3.71 (td, | | |
| | 2-hydroxy-1- | J = 12.0, 11.1, 5.8 Hz, | | |
| | phenylethyl)amino)pyrimidin-4- | 2H), 3.53-3.33 (m, | | |
| | yl)pyridin-2-yl)isoxazol-5-yl)-1- | 2H), 2.85 (s, 3H), 2.68- | | |
| | methylpyrrolidin-2-one | 2.58 (m, 1H), 2.35- | | |
| | | 2.24 (m, 1H). LCMS | | |
| | | (ESI⁺): m/z = 473.4. | | |
| 387 | 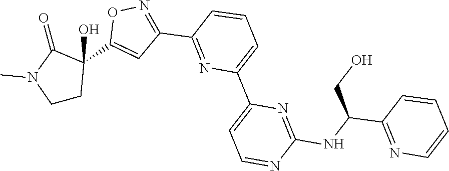 [SMILES: CN1CC[C@@](O)(c2cc(-c3cccc(-c4ccnc(N[C@@H](CO)c5ccccn5)n4)n3)no2)C1=O] | ¹H NMR (401 MHz, DMSO-d₆) δ 8.62- 8.54 (m, 1H), 8.50 (s, 1H), 8.32-8.23 (m, 1H), 8.18-8.04 (m, 2H), 7.77 (td, J = 7.7, 1.9 Hz, 1H), 7.66 (d, J = 5.0 Hz, 1H), 7.60- 7.39 (m, 2H), 7.36- 7.21 (m, 1H), 7.14 (s, 1H), 6.81 (s, 1H), 5.23 | RP HPLC (NH₄OH modifier) | 11 |
| | | (s, 1H), 3.97-3.76 (m, | | |
| | (R)-3-Hydroxy-3-(3-(6-(2-(((R)- | 2H), 3.63-3.31 (m, | | |
| | 2-hydroxy-1-(pyridin-2- | 2H), 2.85 (s, 3H), 2.69 | | |
| | yl)ethyl)amino)pyrimidin-4- | -2.55 (m, 1H), 2.37- | | |
| | yl)pyridin-2-yl)isoxazol-5-yl)-1- | 2.19 (m, 1H). LCMS | | |
| | methylpyrrolidin-2-one | (ESI⁺): m/z = 474.5. | | |
| 388 | 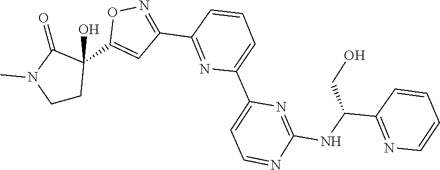 [SMILES: CN1CC[C@@](O)(c2cc(-c3cccc(-c4ccnc(N[C@H](CO)c5ccccn5)n4)n3)no2)C1=O] | ¹H NMR (401 MHz, DMSO-d₆) δ 8.60- 8.54 (m, 1H), 8.54- 8.37 (m, 1H), 8.36- 8.18 (m, 1H), 8.20- 8.03 (m, 3H), 7.84- 7.70 (m, 1H), 7.66 (d, J = 5.0 Hz, 1H), 7.56- 7.42 (m, 2H), 7.35- 7.21 (m, 1H), 7.14 (s, 1H), 6.81 (s, 1H), 5.23 | RP HPLC (NH₄OH modifier) | 10 |
| | | (s, 1H), 5.01 (s, 1H), | | |
| | (R)-3-Hydroxy-3-(3-(6-(2-(((S)- | 3.96-3.78 (m, 2H), | | |
| | 2-hydroxy-1-(pyridin-2- | 3.72-3.30 (m, 2H), | | |
| | yl)ethyl)amino)pyrimidin-4- | 3.18 (s, 1H), 2.85 (s, | | |
| | yl)pyridin-2-yl)isoxazol-5-yl)-1- | 3H), 2.68-2.55 (m, | | |
| | methylpyrrolidin-2-one | 1H), 2.37-2.21 (m, | | |
| | | 1H). LCMS (ESI⁺): | | |
| | | m/z = 474.3. | | |
| 389 | 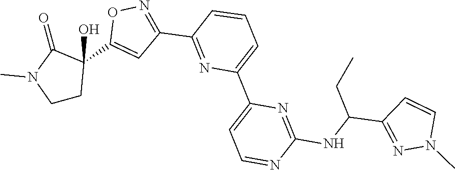 | ¹H NMR (401 MHz, DMSO-d₆) δ 8.55- 8.39 (m, 2H), 8.26- 8.02 (m, 2H), 7.70- 7.62 (m, 1H), 7.59- 7.51 (m, 1H), 7.16 (s, 1H), 6.23 (d, J = 2.2 Hz, 1H), 5.17 (s, 1H), 3.79 (s, 3H), 3.54- 3.39 (m, 2H), 2.86 (s, 3H), 2.68-2.54 (m, | RP HPLC (TFA modifier) | 6 |
| | | 1H), 2.41-2.17 (m, | | |
| | (3R)-3-Hydroxy-1-methyl-3-(3- | 1H), 1.98-1.84 (m, | | |
| | (6-(2-((1-(1-methyl-1H-pyrazol- | 2H), 0.92 (t, J = 7.3 Hz, | | |
| | 3-yl)propyl)amino)pyrimidin-4- | 3H). LCMS (ESI⁺): | | |
| | yl)pyridin-2-yl)isoxazol-5- | m/z = 475.1. | | |
| | yl)pyrrolidin-2-one | | | |
| 390 | 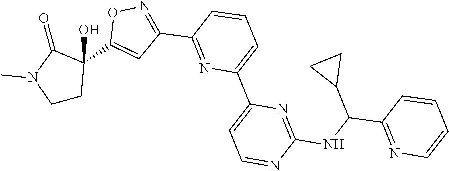 [SMILES: CN1CC[C@@](O)(c2cc(-c3cccc(-c4ccnc(NC(c5ccccn5)C5CC5)n4)n3)no2)C1=O] | ¹H NMR (401 MHz, DMSO-d₆) δ 8.62- 8.49 (m, 1H), 8.49- 8.39 (m, 1H), 8.38- 8.23 (m, 1H), 8.21- 8.04 (m, 2H), 7.91- 7.79 (m, 1H), 7.81- 7.70 (m, 1H), 7.67- 7.46 (m, 2H), 7.30- 7.20 (m, 1H), 7.14- 6.98 (m, 1H), 6.85- | RP HPLC (NH₄OH modifier) | 9 |
| | | 6.74 (m, 1H), 4.65- | | |
| | (3R)-3-(3-(6-(2- | 4.45 (m, 1H), 3.54- | | |
| | ((Cyclopropyl(pyridin-2- | 3.34 (m, 2H), 3.23 (s, | | |
| | yl)methyl)amino)pyrimidin-4- | 1H), 3.18 (d, J = 4.9 | | |
| | yl)pyridin-2-yl)isoxazol-5-yl)-3- | Hz, 1H), 2.92-2.79 | | |
| | hydroxy-1-methylpyrrolidin-2- | (m, 3H), 2.67-2.56 | | |
| | one | (m, 1H), 2.36-2.21 | | |
| | | (m, 1H), 1.48-1.32 | | |
| | | (m, 1H), 1.30-1.13 | | |
| | | (m, 1H), 0.63-0.49 | | |
| | | (m, 1H), 0.49-0.36 | | |
| | | (m, 3H). LCMS (ESI⁺): | | |
| | | m/z = 484.4. | | |
| 391 | 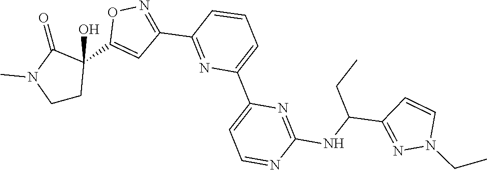 [SMILES: CCC(Nc1nccc(-c2cccc(-c3cc([C@]4(O)CCN(C)C4=O)on3)n2)n1)c1ccn(CC)n1] | ¹H NMR (401 MHz, DMSO-d₆) δ 8.57- 8.42 (m, 2H), 8.21- 8.06 (m, 2H), 7.84- 7.59 (m, 3H), 7.45 (s, 1H), 7.15 (s, 1H), 5.09 (s, 1H), 4.06 (q, J = 7.3 Hz, 2H), 3.52-3.39 (m, 2H), 2.86 (s, 3H), 2.70-2.56 (m, 1H), 2.39-2.23 (m, 1H), | RP HPLC (TFA modifier) | 8 |
| | | 1.98-1.77 (m, 2H), | | |
| | (3R)-3-(3-(6-(2-((1-(1-Ethyl-1H- | 1.32 (t, J = 7.3 Hz, 3H), | | |
| | pyrazol-3- | 0.93 (t, J = 7.3 Hz, 3H). | | |
| | yl)propyl)amino)pyrimidin-4- | LCMS (ESI⁺): m/z = | | |
| | yl)pyridin-2-yl)isoxazol-5-yl)-3- | 489.3. | | |
| | hydroxy-1-methylpyrrolidin-2- | | | |
| | one | | | |
| 392 | 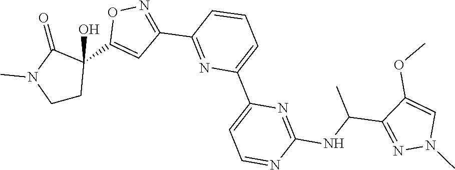 | ¹H NMR (401 MHz, DMSO-d₆) δ 8.56- 8.39 (m, 1H), 8.26- 8.04 (m, 2H), 7.76- 7.60 (m, 1H), 7.41 (s, 1H), 7.16 (s, 1H), 5.34- 5.21 (m, 1H), 3.71 (s, 3H), 3.69 (s, 3H), 3.54- 3.37 (m, 3H), 2.86 (s, 3H), 2.68-2.56 (m, 1H), 2.39-2.24 (m, | RP HPLC (TFA modifier) | 7 |
| | | 1H), 1.58-1.40 (m, | | |
| | (3R)-3-Hydroxy-3-(3-(6-(2-((1- | 3H). LCMS (ESI⁺): | | |
| | (4-methoxy-1-methyl-1H- | m/z = 491.3. | | |
| | pyrazol-3- | | | |
| | yl)ethyl)amino)pyrimidin-4- | | | |
| | yl)pyridin-2-yl)isoxazol-5-yl)-1- | | | |
| | methylpyrrolidin-2-one | | | |
| 393 | 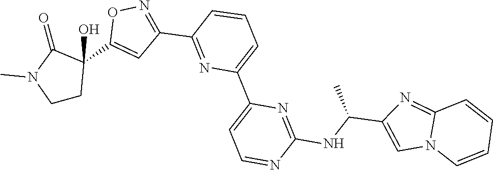 [SMILES: C[C@@H](Nc1nccc(-c2cccc(-c3cc([C@]4(O)CCN(C)C4=O)on3)n2)n1)c1cn2ccccc2n1] | ¹H NMR (401 MHz, DMSO-d₆) δ 8.55- 8.48 (m, 2H), 8.45 (dd, J = 6.8, 2.1 Hz, 1H), 8.19-8.05 (m, 2H), 7.86 (s, 1H), 7.71- 7.60 (m, 2H), 7.60- 7.50 (m, 1H), 7.35- 7.27 (m, 1H), 7.15 (s, 1H), 6.99-6.85 (m, 1H), 6.82 (s, 1H), 5.48 | RP HPLC (NH₄OH modifier) | 10 |
| | | (s, 1H), 3.55-3.39 (m, | | |
| | (R)-3-Hydroxy-3-(3-(6-(2-(((R)- | 2H), 2.85 (s, 3H), 2.68- | | |
| | 1-(imidazo[1,2-a]pyridin-2- | 2.56 (m, 1H), 2.37- | | |
| | yl)ethyl)amino)pyrimidin-4- | 2.20 (m, 1H), 1.63 (d, | | |
| | yl)pyridin-2-yl)isoxazol-5-yl)-1- | J = 6.9 Hz, 3H). LCMS | | |
| | methylpyrrolidin-2-one | (ESI⁺): m/z = 497.2. | | |
| 394 | 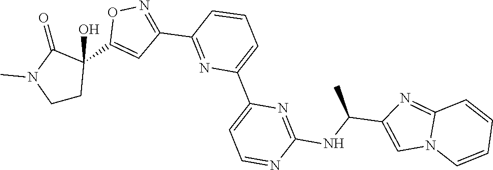 [SMILES: C[C@H](Nc1nccc(-c2cccc(-c3cc([C@]4(O)CCN(C)C4=O)on3)n2)n1)c1cn2ccccc2n1] | ¹H NMR (401 MHz, DMSO-d₆) δ 8.61- 8.47 (m, 2H), 8.47- 8.37 (m, 1H), 8.17- 8.02 (m, 2H), 7.86 (s, 1H), 7.71-7.61 (m, 2H), 7.61-7.46 (m, 1H), 7.34-7.23 (m, 1H), 7.15 (s, 1H), 6.95- 6.86 (m, 1H), 6.83 (s, 1H), 5.48 (s, 1H), 3.52- | RP HPLC (NH₄OH modifier) | 11 |
| | | 3.37 (m, 2H), 2.85 (s, | | |
| | (R)-3-Hydroxy-3-(3-(6-(2-(((S)- | 3H), 2.69-2.53 (m, | | |
| | 1-(imidazo[1,2-a]pyridin-2- | 1H), 2.40-2.13 (m, | | |
| | yl)ethyl)amino)pyrimidin-4- | 1H), 1.63 (d, J = 6.9 | | |
| | yl)pyridin-2-yl)isoxazol-5-yl)-1- | Hz, 3H). LCMS (ESI⁺): | | |
| | methylpyrrolidin-2-one | m/z = 497.3. | | |
| 395 | 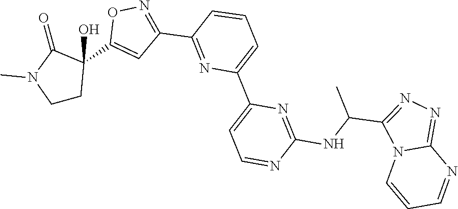 [SMILES: CC(Nc1nccc(-c2cccc(-c3cc([C@]4(O)CCN(C)C4=O)on3)n2)n1)c1nnc2ncccn12] | ¹H NMR (401 MHz, DMSO-d₆) δ 9.40- 9.28 (m, 1H), 8.87- 8.75 (m, 1H), 8.60- 8.46 (m, 1H), 8.18- 8.01 (m, 2H), 7.93- 7.76 (m, 1H), 7.65 (d, J = 5.0 Hz, 1H), 7.38- 7.22 (m, 1H), 7.13 (s, 1H), 6.82 (s, 1H), 5.61 -5.44 (m, 1H), 3.51- 3.37 (m, 1H), 2.85 (s, 3H), 2.68-2.57 (m, | RP HPLC (NH₄OH modifier) | 12 |
| | | 1H), 2.36-2.20 (m, | | |
| | (3R)-3-(3-(6-(2-((1- | 1H), 1.69 (d, J = 7.1 | | |
| | ([1,2,4]Triazolo[4,3-a]pyrimidin- | Hz, 3H). LCMS (ESI⁺): | | |
| | 3-yl)ethyl)amino)pyrimidin-4- | m/z = 499.3. | | |
| | yl)pyridin-2-yl)isoxazol-5-yl)-3- | | | |
| | hydroxy-1-methylpyrrolidin-2- | | | |
| | one | | | |
| 396 | 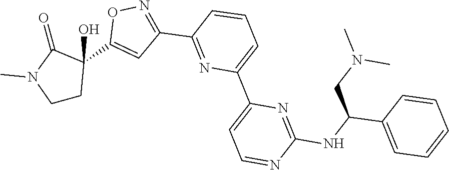 [SMILES: CN(C)C[C@H](Nc1nccc(-c2cccc(-c3cc([C@]4(O)CCN(C)C4=O)on3)n2)n1)c1ccccc1] | ¹H NMR (401 MHz, DMSO-d₆) δ 9.40 (s, 1H), 8.56 (s, 1H), 8.49 (s, 1H), 8.29-8.11 (m, 2H), 8.11-7.99 (m, 1H), 7.74 (d, J = 5.1 Hz, 1H), 7.58 (s, 2H), 7.46-7.34 (m, 2H), 7.34-7.24 (m, 1H), 7.14 (s, 1H), 5.88- 5.56 (m, 1H), 3.66- | RP HPLC (TFA modifier) | 6 |
| | | 3.34 (m, 2H), 2.96- | | |
| | (R)-3-(3-(6-(2-(((R)-2- | 2.88 (m, 6H), 2.85 (s, | | |
| | (Dimethylamino)-1- | 3H), 2.70-2.55 (m, | | |
| | phenylethyl)amino)pyrimidin-4- | 1H), 2.38-2.17 (m, | | |
| | yl)pyridin-2-yl)isoxazol-5-yl)-3- | 1H). LCMS (ESI⁺): | | |
| | hydroxy-1-methylpyrrolidin-2- | m/z = 500.4. | | |
| | one | | | |
| 397 | 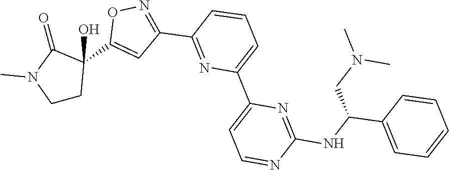 [SMILES: CN(C)C[C@@H](Nc1nccc(-c2cccc(-c3cc([C@]4(O)CCN(C)C4=O)on3)n2)n1)c1ccccc1] | ¹H NMR (401 MHz, DMSO-d₆) δ 9.55- 9.20 (m, 1H), 8.63- 8.54 (m, 1H), 8.54- 8.36 (m, 2H), 8.24- 8.10 (m, 2H), 8.10- 7.97 (m, 1H), 7.80- 7.68 (m, 1H), 7.68- 7.47 (m, 2H), 7.47- 7.33 (m, 2H), 7.33- 7.30 (m, 1H), 7.14 (s, | RP HPLC (TFA modifier) | 6 |
| | | 1H), 5.75 (s, 1H), 3.88- | | |
| | (R)-3-(3-(6-(2-(((S)-2- | 3.37 (m, 2H), 2.96- | | |
| | (Dimethylamino)-1- | 2.87 (m, 6H), 2.85 (s, | | |
| | phenylethyl)amino)pyrimidin-4- | 3H), 2.68-2.54 (m, | | |
| | yl)pyridin-2-yl)isoxazol-5-yl)-3- | 1H), 2.38-2.20 (m, | | |
| | hydroxy-1-methylpyrrolidin-2- | 1H). LCMS (ESI⁺): | | |
| | one | m/z = 500.6. | | |
| 398 | 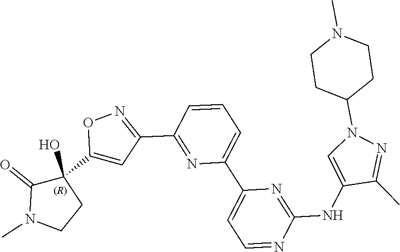 | ¹H NMR (500 MHz, DMSO-d₆) δ 9.02- 8.96 (m, 1H), 8.68- 8.63 (m, 1H), 8.38- 8.33 (m, 1H), 8.28- 8.21 (m, 2H), 7.71- 7.67 (m, 1H), 7.67- 7.59 (m, 1H), 7.22 (s, 1H), 6.78 (s, 1H), 6.71- 6.67 (m, 1H), 3.53- 3.42 (m, 2H), 3.04- 2.99 (m, 3H), 2.86 (s, 3H), 2.66-2.59 (m, 1H), 2.37-2.27 (m, 1H). MS (ESI+): m/z = 433.1. | RP HPLC (TFA modifier) | 23 |
| | (R)-3-Hydroxy-1-methyl-3-(3- | | | |
| | (6-(2-((1-methyl-1H-imidazol-2- | | | |
| | yl)amino)pyrimidin-4-yl)pyridin- | | | |
| | 2-yl)isoxazol-5-yl)pyrrolidin-2- | | | |
| | one | | | |
| 399 | 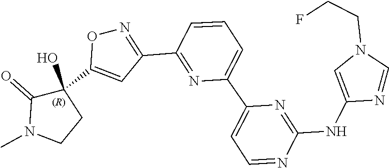 | ¹H NMR (500 MHz, DMSO-d₆) δ 10.39 (s, 1H), 8.78-8.72 (m, 1H), 8.59-8.51 (m, 1H), 8.50-8.46 (m, 1H), 8.23-8.17 (m, 2H), 7.99-7.95 (m, 1H), 7.68-7.62 (m, 1H), 7.18 (s, 1H), 6.77 (s, 1H), 4.91-4.86 (m, | RP HPLC (TFA modifier) | 26 |
| | | 1H), 4.81-4.76 (m, | | |
| | (R)-3-(3-(6-(2-((1-(2- | 1H), 4.58-4.53 (m, | | |
| | Fluoroethyl)-1H-imidazol-4- | 1H), 4.52-4.46 (m, | | |
| | yl)amino)pyrimidin-4-yl)pyridin- | 1H), 2.85 (s, 3H), 2.66- | | |
| | 2-yl)isoxazol-5-yl)-3-hydroxy-1- | 2.59 (m, 1H), 2.34- | | |
| | methylpyrrolidin-2-one | 2.27 (m, 1H). MS | | |
| | | (ESI+): m/z = 465.2. | | |
| 400 and 401 | 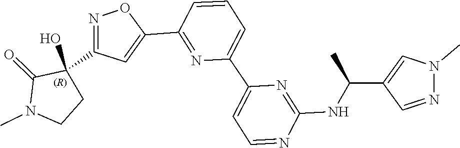 | Example 400: ¹H NMR (400 MHz, DMSO-d₆) δ 8.54-8.48 (m, 1H), 8.47-8.37 (m, 1H), 8.22-8.15 (m, 1H), 8.11-8.05 (m, 1H), 7.63-7.58 (m, 2H), 7.57-7.52 (m, 1H), 7.40 (s, 1H), 7.31 (s, | FCC (DCM/Me OH) then SFC (DAICEL CHIRALCEL OJ column, 10 μm, 250 × 30 mm; | Ex 400: 26 |
| | | 1H), 6.57 (s, 1H), 5.34- | 35% (v/v) | |
| | 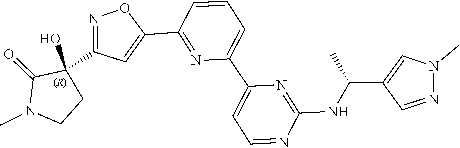 | 5.17 (m, 1H), 3.76 (s, 3H), 3.51-3.38 (m, 2H), 2.81 (s, 3H), 2.75- 2.67 (m, 1H), 2.30- 2.21 (m, 1H), 1.52- 1.44 (m, 3H). MS (ESI⁺): m/z = 461.2. Example 401: ¹H NMR (400 MHz, DMSO- | EtOH (containing 0.1% NH₃ in H₂O)/CO₂)). The first eluting peak was designated Example | Ex. 401: 28 |
| | | d₆) δ 8.53-8.48 (m, | 400 and | |
| | (R)-3-Hydroxy-1-methyl-3-(5- | 1H), 8.47-8.38 (m, | the second | |
| | (6-(2-(((S)-1-(1-methyl-1H- | 1H), 8.21-8.16 (m, | eluting | |
| | pyrazol-4- | 1H), 8.10-8.06 (m, | peak was | |
| | yl)ethyl)amino)pyrimidin-4- | 1H), 7.63-7.58 (m, | designated | |
| | yl)pyridin-2-yl)isoxazol-3- | 2H), 7.57-7.51 (m, | Example | |
| | yl)pyrrolidin-2-one and (S)-3- | 1H), 7.39 (s, 1H), 7.31 | 401. | |
| | hydroxy-1-methyl-3-(5-(6-(2- | (s, 1H), 6.57 (s, 1H), | | |
| | (((S)-1-(1-methyl-1H-pyrazol-4- | 5.35-5.16 (m, 1H), | | |
| | yl)ethyl)amino)pyrimidin-4- | 3.76 (s, 3H), 3.50- | | |
| | yl)pyridin-2-yl)isoxazol-3- | 3.37 (m, 2H), 2.81 (s, | | |
| | yl)pyrrolidin-2-one | 3H), 2.75-2.67 (m, | | |
| | | 1H), 2.30-2.21 (m, | | |
| | | 1H), 1.52-1.45 (m, | | |
| | | 3H). MS (ESI⁺): m/z = | | |
| | | 461.2. | | |
| 402 | 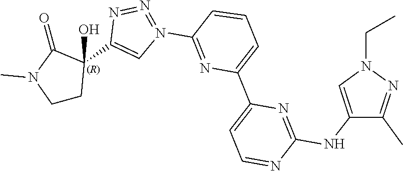 | ¹H NMR (400 MHz, MeOH-d₄) δ 8.98 (s, 1H), 8.58-8.49 (m, 2H), 8.36-8.24 (m, 2H), 7.98-7.91 (m, 2H), 4.24-4.15 (m, 2H), 3.69-3.60 (m, 2H), 3.01-2.92 (m, 4H), 2.51-2.41 (m, 1H), 2.27 (s, 3H), 1.54- 1.48 (m, 3H). MS | RP HPLC (TFA modifier) | 25 |
| | | (ESI⁺): m/z = 461.2. | | |
| | (R)-3-(1-(6-(2-((1-Ethyl-3- | | | |
| | methyl-1H-pyrazol-4- | | | |
| | yl)amino)pyrimidin-4-yl)pyridin- | | | |
| | 2-yl)-1H-1,2,3-triazol-4-yl)-3- | | | |
| | hydroxy-1-methylpyrrolidin-2- | | | |
| | one | | | |
| 403 | 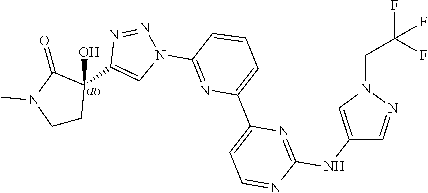 | ¹H NMR (400 MHz, MeOH-d₄) δ 8.98 (s, 1H), 8.65-8.59 (m, 1H), 8.59-8.50 (m, 1H), 8.33-8.16 (m, 3H), 7.88-7.81 (m, 1H), 7.79 (s, 1H), 3.71- 3.57 (m, 3H), 2.99 (s, 5H), 2.51-2.41 (m, 1H). MS (ESI⁺): m/z = 501.1. | RP HPLC (TFA modifier) | 6 |
| | (R)-3-Hydroxy-1-methyl-3-(1- | | | |
| | (6-(2-((1-(2,2,2-trifluoroethyl)- | | | |
| | 1H-pyrazol-4- | | | |
| | yl)amino)pyrimidin-4-yl)pyridin- | | | |
| | 2-yl)-1H-1,2,3-triazol-4- | | | |
| | yl)pyrrolidin-2-one | | | |
| 404 and 405 | 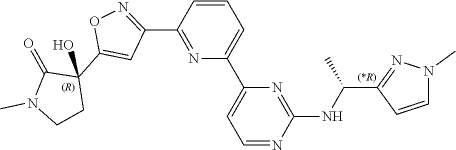 | Example 404: LCMS (ESI⁺): Mass calcd. for C₂₃H₂₄N₈O₃ 460.2 m/z, found 461.0. Example 405: LCMS (ESI⁺): Mass calcd. for C₂₃H₂₄N₈O₃ 460.2 m/z, found 461.0. [M + H]⁺. | SFC (Stationary phase: Chiralcel OJ-H 5 μm 250 × 21 mm, Mobile phase: 25% methanol, 75% CO₂). | Ex. 404: 18 Ex 405: 18 |
| | | | Example | |
| | 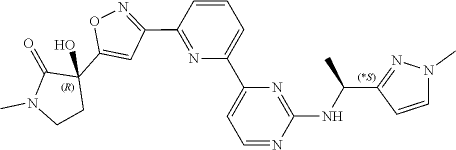 | | 404 was the first eluting peak, and example 405 was the second eluting peak. | |
| | (R)-3-Hydroxy-1-methyl-3-(3- | | | |
| | (6-(2-(((*R)-1-(1-methyl-1H- | | | |
| | pyrazol-3- | | | |
| | yl)ethyl)amino)pyrimidin-4- | | | |
| | yl)pyridin-2-yl)isoxazol-5- | | | |
| | yl)pyrrolidin-2-one and (R)-3- | | | |
| | hydroxy-1-methyl-3-(3-(6-(2- | | | |
| | (((*S)-1-(1-methyl-1H-pyrazol- | | | |
| | 3-yl)ethyl)amino)pyrimidin-4- | | | |
| | yl)pyridin-2-yl)isoxazol-5- | | | |
| | yl)pyrrolidin-2-one | | | |

The compounds shown in Table 6 were prepared in a manner analogous to (R)-3-(5-(6-(2-((1-ethyl-1H-pyrazol-4-yl)amino)pyrimidin-4-yl)pyridin-2-yl)isoxazol-3-yl)-3-hydroxy-1-methylpyrrolidin-2-one (Example 91) using the appropriate amine in place of 1-ethyl-1H-pyrazol-4-amine and appropriate sulfone in place of (R)-3-hydroxy-1-methyl-3-[5-[6-(2-methylsulfonylpyrimidin-4-yl)-2-pyridyl]isoxazol-3-yl]pyrrolidin-2-one. Purification methods are noted in the table.

TABLE 6

| | Structure | | Purification | Yield |
|---|---|---|---|---|
| Ex | Name | Analytical data | conditions | (%) |
| 406 | 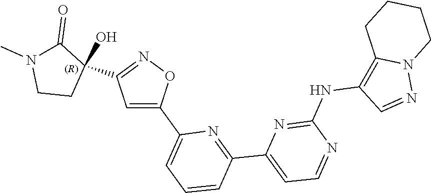 | ¹H NMR (500 MHz, DMSO-d₆) δ 8.94 (s, 1H), 8.57 (d, J = 5.1 Hz, 1H), 8.36 (s, 1H), 8.21 (t, J = 7.8 Hz, 1H), 8.12-8.08 (m, 1H), 7.71 (d, J = 5.0 Hz, 1H), 7.33 (s, 1H), 6.56 (s, 1H), 4.06 (t, J = 6.1 Hz, 2H), 3.46 (dd, J = 8.3, 4.1 Hz, 1H), 3.43- 3.39 (m, 1H), 2.82 (s, 3H), 2.71 (t, J = 6.4 Hz, | RP HPLC (NH₄OH Modifier) | 10 |
| | (R)-3-Hydroxy-1-methyl-3-(5-(6-(2-((4,5,6,7-tetrahydropyrazolo | 3H), 2.29-2.24 (m, 2H), | | |
| | [1,5-a]pyridin-3-yl)amino)pyrimidin-4-yl)pyridin- | 2.00-1.95 (m, 3H), 1.80 | | |
| | 2-yl)isoxazol-3-yl)pyrrolidin-2-one | (d, J = 6.6 Hz, 2H), 1.25 | | |
| | | (s, 3H). MS (ESI⁺): m/z = | | |
| | | 473.3. | | |
| 407 | 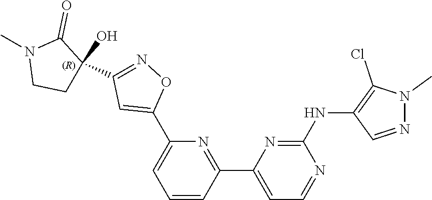 | ¹H NMR (500 MHz, DMSO-d₆) δ 9.06 (s, 1H), 8.60 (d, J = 5.0 Hz, 1H), 8.39-8.31 (m, 1H), 8.20 (t, J = 7.8 Hz, 1H), 8.12- 8.08 (m, 1H), 7.81-7.75 (m, 2H), 7.33 (s, 1H), 6.56 (s, 1H), 3.84 (s, 3H), 3.50-3.44 (m, 1H), 3.44- 3.38 (m, 1H), 2.82 (s, 3H), 2.75-2.68 (m, 1H), 2.31-2.22 (m, 1H). MS | RP HPLC (NH₄OH Modifier) | 32 |
| | (R)-3-(5-(6-(2-((5-Chloro-1-methyl-1H-pyrazol-4-yl)amino)pyrimidin- | (ESI⁺): m/z = 467.2. | | |
| | 4-yl)pyridin-2-yl)isoxazol-3-yl)-3-hydroxy-1-methylpyrrolidin-2-one | | | |
| 408 | 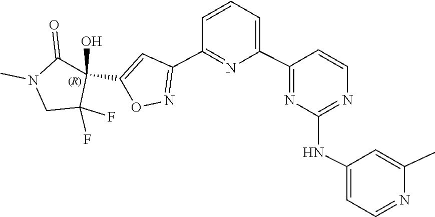 | ¹H NMR (500 MHz, DMSO-d₆) δ 11.47 (s, 1H), 8.97 (d, J = 5.1 Hz, 1H), 8.57-8.55 (m, 1H), 8.55-8.53 (m, 1H), 8.30 (t, J = 4.0 Hz, 1H), 8.29- 8.26 (m, 2H), 8.17 (s, 2H), 7.99 (s, 1H), 7.38 (s, 1H), 4.04 (t, J = 12.3 Hz, 2H), 2.98 (s, 3H), 2.67 (s, 3H). MS (ESI⁺): m/z = 480.2. | RP HPLC (TFA modifier) | 24 |
| | (R)-4,4-Difluoro-3-hydroxy-1-methyl-3-(3-(6-(2-((2-methylpyridin-4- | | | |
| | yl)amino)pyrimidin-4-yl)pyridin-2-yl)isoxazol-5-yl)pyrrolidin-2-one | | | |
| 409 | 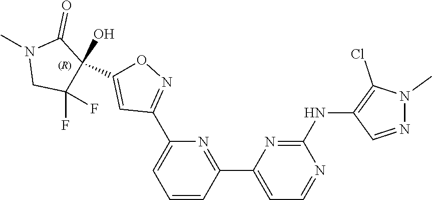 | ¹H NMR (500 MHz, DMSO-d₆) δ 9.06 (s, 1H), 8.60 (d, J = 5.0 Hz, 1H), 8.38 (s, 1H), 8.22-8.18 (m, 1H), 8.18-8.16 (m, 1H), 7.96 (s, 1H), 7.82 (d, J = 5.0 Hz, 1H), 7.79 (s, 1H), 7.31 (s, 1H), 4.03 (t, J = 12.9, 11.6 Hz, 3H), 3.84 (s, 1H), 2.97 (s, 3H). MS (ESI⁺): m/z = 503.1. | RP HPLC (TFA modifier) | 24 |
| | (R)-3-(3-(6-(2-((5-Chloro-1-methyl-1H-pyrazol-4- | | | |
| | yl)amino)pyrimidin-4-yl)pyridin-2-yl)isoxayl)zol-5-yl)-4,4-difluoro-3- | | | |
| | hydroxy-1-methylpyrrolidin-2-one | | | |
| 410 | 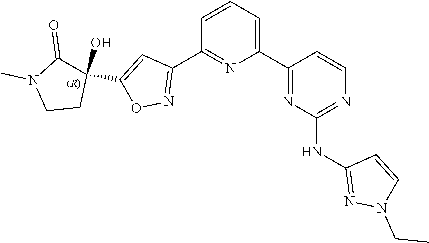 | ¹H NMR (500 MHz, DMSO-d₆) δ 9.90 (s, 1H), 8.65 (d, J = 5.0 Hz, 1H), 8.47 (dd, J = 7.7, 1.2 Hz, 1H), 8.21 (t, J = 7.7 Hz, 1H), 8.16 (dd, J = 7.8, 1.2 Hz, 1H), 7.82 (d, J = 5.0 Hz, 1H), 7.67 (d, J = 2.3 Hz, 1H), 7.18 (s, 1H), 6.77 (s, 1H), 6.73 (d, J = 2.2 Hz, 1H), 4.07 (q, J = 7.2 Hz, 2H), 3.53-3.48 (m, 1H), 3.48-3.44 (m, 1H), 2.86 (s, 3H), 2.66- | RP HPLC (NH₄OH Modifier) | 21 |
| | (R)-3-(3-(6-(2-((1-Ethyl-1H-pyrazol-3-yl)amino)pyrimidin-4- | 2.60 (m, 1H), 2.34-2.27 | | |
| | yl)pyridin-2-yl)isoxazol-5-yl)-3-hydroxy-1-methylpyrrolidin-2-one | (m, 1H), 1.39 (t, J = 7.3 | | |
| | | Hz, 3H). MS (ESI⁺): m/z = | | |
| | | 447.1. | | |
| 411 | 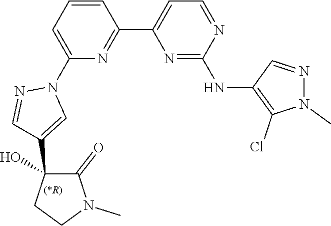 | ¹H NMR (400 MHz, DMSO-d₆) δ 9.06 (br s, 1H), 8.80 (s, 1H), 8.61 (d, J = 4.8 Hz, 1H), 8.26- 8.13 (m, 2H), 8.09-7.98 (m, 1H), 7.87 (s, 1H), 7.81 (d, J = 4.8 Hz, 1H), 7.80-7.76 (m, 1H), 6.01 (s, 1H), 3.83 (s, 3H), 3.47- 3.38 (m, 2H), 2.80 (s, 3H), 2.61-2.54 (m, 1H), 2.30-2.18 (m, 1H). MS (ESI⁺): m/z = 466.1. | RP HPLC (NH₄OH Modifier) | 11 |
| | (*R)-3-(1-(6-(2-((5-Chloro-1-methyl-1H-pyrazol-4- | | | |
| | yl)amino)pyrimidin-4-yl)pyridin-2-yl)-1H-pyrazol-4-yl)-3-hydroxy-1- | | | |
| | methylpyrrolidin-2-one | | | |

The compounds shown in Table 7 was prepared in a manner analogous to Example 15 using the appropriate pyrimidinyl chloride in Step A and the appropriate amines in Step B in place of 2,4-dichloropyrimidine in Step A and 6,7-dihydro-5H-pyrazolo[5,1-b][1,3]oxazin-3-amine in Step B. Final purification methods are noted in the table.

TABLE 7

| | Structure | | Purification | Yield |
|---|---|---|---|---|
| Ex. | Name | Analytical data | conditions | (%) |
| 412 | 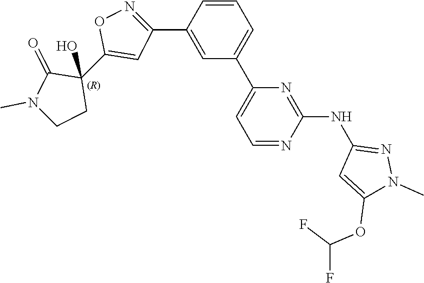 | ¹H NMR (600 MHz, Methanol-d₄) δ 8.77 (t, J = 1.8 Hz, 1H), 8.59 (d, J = 6.0 Hz, 1H), 8.40-8.24 (m, 1H), 8.15-8.04 (m, 1H), 7.80-7.63 (m, 2H), 7.23-6.89 (m, 2H), 6.28 (s, 1H), 3.76 (s, 3H), 3.69-3.54 (m, 2H), 3.00 (s, 3H), 2.82- 2.74 (m, 1H), 2.49- 2.35 (m, 1H). MS (ESI⁺): m/z = 498.1. | RP HPLC (NH₄OH modifier) | 31 |
| | (R)-3-(3-(3-(2-((5-(Difluoromethoxy)-1-methyl-1H-pyrazol-3- | | | |
| | yl)amino)pyrimidin-4-yl)phenyl)isoxazol-5-yl)-3-hydroxy-1- | | | |
| | methylpyrrolidin-2-one | | | |
| 413 | 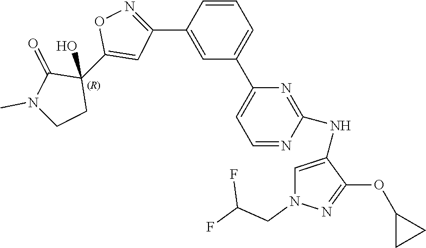 | ¹H NMR (500 MHz, Methanol-d₄) δ 8.70 (t, J = 1.8 Hz, 1H), 8.40 (d, J = 6.0 Hz, 1H), 8.29 (d, J = 7.9 Hz, 1H), 8.08 (d, J = 7.8 Hz, 1H), 7.98 (s, 1H), 7.69 (t, J = 7.8 Hz, 1H), 7.56 (d, J = 6.0 Hz, 1H), 6.99 (s, 1H), 6.40-6.06 (m, 1H), 4.54-4.41 (m, 2H), 4.26-4.11 (m, 1H), 3.66- 3.53 (m, 2H), 2.99 (s, 3H), 2.83-2.67 (m, | RP HPLC (TFA modifier) | 18 |
| | (R)-3-(3-(3-(2-((3-Cyclopropoxy-1-(2,2-difluoroethyl)-1H-pyrazol- | 1H), 2.52-2.35 (m, | | |
| | 4-yl)amino)pyrimidin-4-yl)phenyl)isoxazol-5-yl)-3-hydroxy-1- | 1H), 0.86-0.69 (m, | | |
| | methylpyrrolidin-2-one | 4H). MS (ESI⁺): m/z = | | |
| | | 538.5. | | |
| 414 | 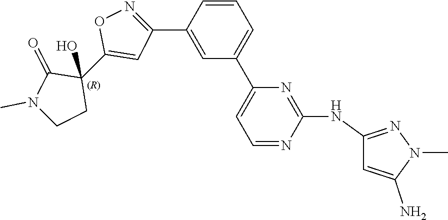 | ¹H NMR (500 MHz, Methanol-d₄) δ 8.72- 8.63 (m, 2H), 8.33- 8.23 (m, 1H), 8.09- 7.98 (m, 1H), 7.73- 7.61 (m, 2H), 7.00 (s, 1H), 3.77-3.55 (m, 5H), 3.00 (s, 3H), 2.83- 2.69 (m, 1H), 2.48- 2.39 (m, 1H). MS (ESI⁺): m/z = 447.4. | RP HPLC (TFA modifier) | 7 |
| | (R)-3-(3-(3-(2-((5-Amino-1-methyl-1H-pyrazol-3-yl)amino)pyrimidin-4- | | | |
| | yl)phenyl)isoxazol-5-yl)-3-hydroxy-1-methylpyrrolidin-2-one | | | |
| 415 | 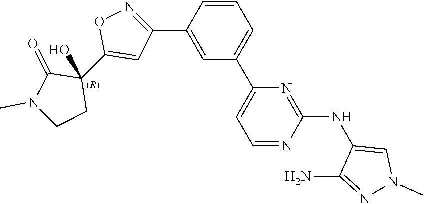 | ¹H NMR (500 MHz, Methanol-d₄) δ 8.58 (d, J = 5.4 Hz, 1H), 8.32- 8.20 (m, 1H), 8.06- 7.96 (m, 1H), 7.84 (s, 1H), 7.69 (t, J = 7.8 Hz, 1H), 7.51 (d, J = 5.4 Hz, 1H), 6.99 (d, J = 7.3 Hz, 1H), 3.88 (s, 3H), 3.67-3.56 (m, 2H), 2.99 (s, 3H), 2.83- 2.73 (m, 1H), 2.49- | RP HPLC (TFA modifier) | 8 |
| | (R)-3-(3-(3-(2-((3-Amino-1-methyl-1H-pyrazol-4-yl)amino)pyrimidin-4- | 2.36 (m, 1H). MS | | |
| | yl)phenyl)isoxazol-5-yl)-3-hydroxy-1-methylpyrrolidin-2-one | (ESI⁺): m/z = 447.4. | | |
| 416 | 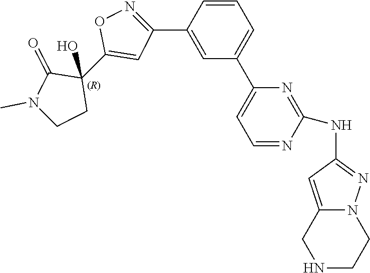 | ¹H NMR (500 MHz, Methanol-d₄) δ 8.73 (t, J = 1.8 Hz, 1H), 8.56 (d, J = 5.6 Hz, 1H), 8.35-8.19 (m, 1H), 8.12-7.97 (m, 1H), 7.68 (t, J = 7.8 Hz, 1H), 7.55 (d, J = 5.6 Hz, 1H), 6.99 (s, 1H), 6.73 (s, 1H), 4.66-4.57 (m, 2H), 4.42 (dd, J = 6.5, 5.2 Hz, 2H), 3.86 (dd, J = 6.6, 5.2 Hz, 2H), 3.68- 3.52 (m, 2H), 3.00 (s, 3H), 2.88-2.76 (m, 1H), 2.50-2.35 (m, | RP HPLC (TFA modifier) | 14 |
| | (R)-3-Hydroxy-1-methyl-3-(3-(3-(2-((4,5,6,7- | 1H). MS (ESI⁺): m/z = | | |
| | tetrahydropyrazolo[1,5-a]pyrazin-2-yl)amino)pyrimidin-4- | 472.6. | | |
| | yl)phenyl)isoxazol-5-yl)pyrrolidin-2-one | | | |
| 417 | 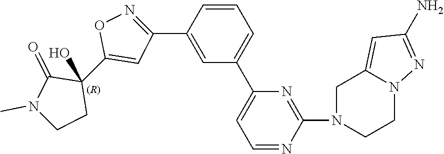 | ¹H NMR (500 MHz, Methanol-d₄) δ 8.73- 8.57 (m, 1H), 8.57- 8.46 (m, 1H), 8.29- 8.17 (m, 1H), 8.05- 7.89 (m, 1H), 7.64 (t, J = 7.8 Hz, 1H), 7.32 (d, J = 5.2 Hz, 1H), 7.00 (s, 1H), 5.12 (s, 2H), | RP HPLC (TFA modifier) | 7 |
| | (R)-3-(3-(3-(2-(2-Amino-6,7-dihydropyrazolo[1,5-a]pyrazin-5(4H)- | 4.57-4.37 (m, 2H), | | |
| | yl)pyrimidin-4-yl)phenyl)isoxazol-5-yl)-3-hydroxy-1-methylpyrrolidin- | 4.20 (t, J = 5.5 Hz, 2H), | | |
| | 2-one | 3.73-3.54 (m, 2H), | | |
| | | 3.00 (s, 3H), 2.87- | | |
| | | 2.73 (m, 1H), 2.51- | | |
| | | 2.29 (m, 1H). MS | | |
| | | (ESI⁺): m/z = 472.6. | | |
| 418 | 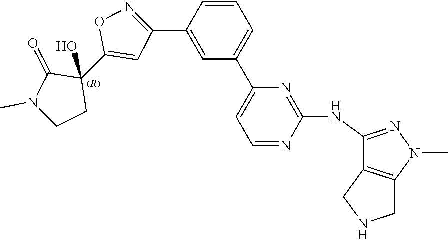 | ¹H NMR (500 MHz, Methanol-d₄) δ 8.70 (s, 1H), 8.51 (d, J = 5.3 Hz, 1H), 8.30 (d, J = 8.1 Hz, 1H), 8.00 (d, J = 7.8 Hz, 1H), 7.67 (t, J = 7.8 Hz, 1H), 7.36 (d, J = 5.3 Hz, 1H), 7.00 (s, 1H), 4.80-4.60 (m, 4H), 3.81 (s, 3H), 3.69- 3.53 (m, 2H), 3.00 (s, 3H), 2.83-2.71 (m, 1H), 2.52-2.32 (m, 1H). MS (ESI⁺): m/z = | RP HPLC (TFA modifier) | 9 |
| | (R)-3-Hydroxy-1-methyl-3-(3-(3-(2-((1-methyl-1,4,5,6- | 473.3. | | |
| | tetrahydropyrrolo[3,4-c]pyrazol-3-yl)amino)pyrimidin-4- | | | |
| | yl)phenyl)isoxazol-5-yl)pyrrolidin-2-one | | | |
| 419 | 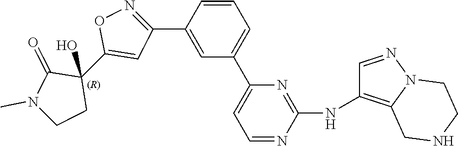 | ¹H NMR (600 MHz, Methanol-d₄) δ 8.63 (s, 1H), 8.42 (d, J = 5.3 Hz, 1H), 8.21 (d, J = 7.6 Hz, 1H), 8.02- 7.94 (m, 1H), 7.71- 7.62 (m, 2H), 7.38- 7.26 (m, 1H), 6.97 (s, | RP HPLC (NH₄OH modifier) | 12 |
| | (R)-3-Hydroxy-1-methyl-3-(3-(3-(2-((4,5,6,7-tetrahydropyrazolo[1,5- | 1H), 4.21-4.11 (m, | | |
| | a]pyrazin-3-yl)amino)pyrimidin-4-yl)phenyl)isoxazol-5-yl)pyrrolidin-2-one | 2H), 4.00 (s, 2H), 3.64- | | |
| | | 3.56 (m, 2H), 3.40- | | |
| | | 3.35 (m, 2 H), 3.00 (s, | | |
| | | 3H), 2.80-2.73 (m, | | |
| | | 1H), 2.50-2.39 (m, | | |
| | | 1H). MS (ESI⁺): m/z = | | |
| | | 473.8. | | |
| 420 | 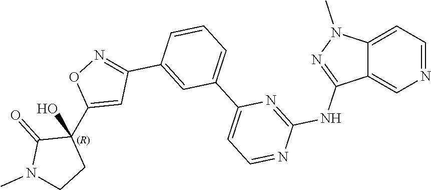 | ¹H NMR (600 MHz, Methanol-d₄) δ 9.87- 9.78 (m, 1H), 8.69 (d, J = 5.3 Hz, 1H), 8.57 (t, J = 1.8 Hz, 1H), 8.43 (dd, J = 7.0, 0.8 Hz, 1H), 8.29-8.21 (m, 1H), 8.06 (dd, J = 7.0, 0.9 Hz, 1H), 8.04-7.99 (m, 1H), 7.71-7.58 (m, 2H), 6.87 (s, 1H), | RP HPLC (TFA modifier) | 9 |
| | (R)-3-Hydroxy-1-methyl-3-(3-(3-(2-((1-methyl-1H-pyrazolo[4,3-c]pyridin- | 4.20 (s, 3H), 3.71- | | |
| | 3-yl)amino)pyrimidin-4-yl)phenyl)isoxazol-5-yl)pyrrolidin-2-one | 3.55 (m, 2H), 3.00 (s, | | |
| | | 3H), 2.81-2.75 (m, | | |
| | | 1H), 2.50-2.38 (m, | | |
| | | 1H). MS (ESI⁺): m/z = | | |
| | | 483.3. | | |
| 421 | 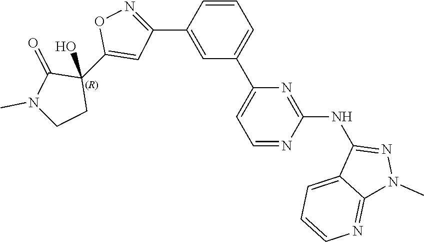 | ¹H NMR (600 MHz, Methanol-d₄) δ 8.70 (s, 1H), 8.66-8.61 (m, 2H), 8.43 (dd, J = 8.2, 1.6 Hz, 1H), 8.32 (d, J = 7.9 Hz, 1H), 8.13- 8.01 (m, 1H), 7.76- 7.62 (m, 2H), 7.25 (dd, J = 8.1, 4.5 Hz, 1H), 6.92 (s, 1H), 4.16 (s, 3H), 3.68-3.54 (m, 2H), 3.00 (s, 4H), 2.81- 2.70 (m, 1H), 2.51- 2.36 (m, 1H). | RP HPLC (NH₄OH modifier) | 16 |
| | (R)-3-Hydroxy-1-methyl-3-(3-(3-(2-((1-methyl-1H-pyrazolo[3,4- | MS (ESI⁺): m/z = | | |
| | b]pyridin-3-yl)amino)pyrimidin-4-yl)phenyl)isoxazol-5-yl)pyrrolidin-2-one | 483.3. | | |
| 422 | 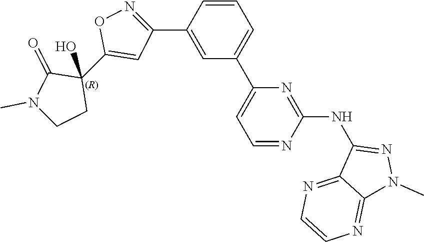 | ¹H NMR (600 MHz, Methanol-d₄) δ 8.64 (d, J = 2.3 Hz, 1H), 8.56 (dd, J = 3.0, 2.0 Hz, 2H), 8.52 (d, J = 5.3 Hz, 1H), 8.24-8.17 (m, 1H), 8.02-7.93 (m, 1H), 7.61 (t, J = 7.8 Hz, 1H), 7.50 (d, J = 5.3 Hz, 1H), 6.86 (s, 1H), 4.16 (s, 4H), 3.65- 3.56 (m, 2H), 3.00 (s, 4H), 2.80-2.71 (m, 1H), 2.49-2.39 (m, | RP HPLC (NH₄OH modifier) | 3 |
| | (R)-3-Hydroxy-1-methyl-3-(3-(3-(2-((1-methyl-1H-pyrazolo[3,4-b]pyrazin- | 1H). MS (ESI⁺): m/z = | | |
| | 3-yl)amino)pyrimidin-4-yl)phenyl)isoxazol-5-yl)pyrrolidin-2-one | 483.3. | | |
| 423 | 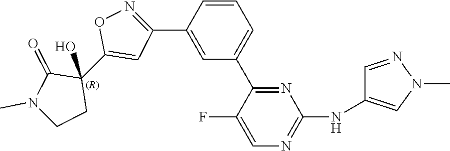 | ¹H NMR (500 MHz, Methanol-d₄) δ 8.66 (s, 1H), 8.45 (d, J = 3.7 Hz, 1H), 8.28-8.20 (m, 1H), 8.09-7.97 (m, 2H), 7.70 (d, J = 7.8 Hz, 1H), 7.66 (d, J = 17.0 Hz, 1H), 6.97 (s, 1H), 3.93 (s, 3H), 3.67- | RP HPLC (TFA modifier) | 40 |
| | (R)-3-(3-(3-(5-Fluoro-2-((1-methyl-1H-pyrazol-4-yl)amino)pyrimidin-4- | 3.54 (m, 2H), 2.99 (s, | | |
| | yl)phenyl)isoxazol-5-yl)-3-hydroxy-1-methylpyrrolidin-2-one | 3H), 2.82-2.71 (m, | | |
| | | 1H), 2.49-2.38 (m, | | |
| | | 1H). MS (ESI⁺): m/z = | | |
| | | 450.1. | | |
| 424 | 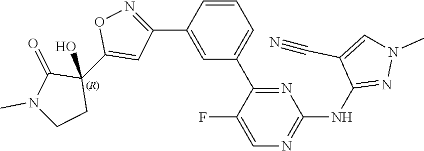 | ¹H NMR (500 MHz, Methanol-d₄) δ 8.65 (t, J = 1.8 Hz, 1H), 8.49 (d, J = 3.6 Hz, 1H), 8.24 (d, J = 6.2 Hz, 2H), 8.06-7.98 (m, 1H), 7.70-7.60 (m, 1H), 6.96 (s, 1H), 3.92 (s, 3H), 3.66-3.52 (m, | RP HPLC (NH₄OH modifier) | 10 |
| | (R)-3-((5-Fluoro-4-(3-(5-(3-hydroxy-1-methyl-2-oxopyrrolidin-3- | 2H), 3.00 (s, 3H), 2.82- | | |
| | yl)isoxazol-3-yl)phenyl)pyrimidin-2-yl)amino)-1-methyl-1H-pyrazole-4- | 2.73 (m, 1H), 2.50- | | |
| | carbonitrile | 2.36 (m, 1H). MS | | |
| | | (ESI⁺): m/z = 475.2. | | |
| 425 | 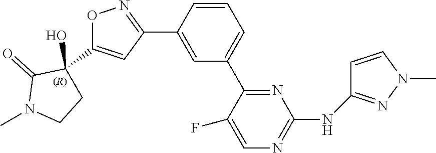 | 1H NMR (500 MHz, Methanol-d₄) δ 8.64 (s, 1H), 8.50 (dd, J = 3.6, 1.6 Hz, 1H), 8.29- 8.19 (m, 1H), 8.10- 8.01 (m, 1H), 7.69 (t, J = 7.8 Hz, 1H), 7.57 (d, J = 7.6 Hz, 1H), 6.96 (d, J = 1.0 Hz, 1H), | RP HPLC (TFA modifier) | 22 |
| | (R)-3-(3-(3-(5-Fluoro-2-((1-methyl-1H-pyrazol-3-yl)amino)pyrimidin-4- | 6.70-6.63 (m, 1H), | | |
| | yl)phenyl)isoxazol-5-yl)-3-hydroxy-1-methylpyrrolidin-2-one | 3.87 (s, 3H), 3.67- | | |
| | | 3.53 (m, 2H), 2.99 (s, | | |
| | | 3H), 2.83-2.70 (m, | | |
| | | 1H), 2.49-2.36 (m, | | |
| | | 1H). MS (ESI⁺): m/z = | | |
| | | 450.3. | | |

The compounds shown below in Table 8 were prepared in a manner analogous to (R)-3-(3-(3-(2-((6,7-dihydro-5H-pyrazolo[5,1-b][1,3]oxazin-3-yl)amino)pyrimidin-4-yl)phenyl)isoxazol-5-yl)-3-hydroxy-1-methylpyrrolidin-2-one (Example 15) using 3-bromo-2-fluorobenzaldehyde for the intermediate preparation instead of 3-bromobenzaldehyde for Intermediate 11, and using the appropriate amines in place of 6,7-dihydro-5H-pyrazolo[5,1-b][1,3]oxazin-3-amine.

TABLE 8

| | Structure | | Purification | Yield |
|---|---|---|---|---|
| Ex. | Name | Analytical data | conditions | (%) |
| 426 | 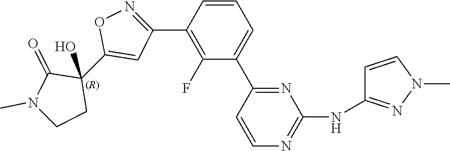 | ¹H NMR (500 MHz, Methanol-d₄) δ 8.62 (d, J = 5.9 Hz, 1H), 8.32- 8.24 (m, 1H), 8.19- 8.08 (m, 1H), 7.63 (d, J =2.3 Hz, 1H), 7.53 (t, J = 7.8 Hz, 2H), 6.96 (d, J = 3.4 Hz, 1H), 6.46 (s, 1H), 3.93 (s, 3H), | RP HPLC (NH₄OH modifier) | 18 |
| | (R)-3-(3-(2-Fluoro-3-(2-((1-methyl-1H-pyrazol-3- | 3.68-3.54 (m, 2H), | | |
| | yl)amino)pyrimidin-4-yl)phenyl)isoxazol-5-yl)-3- | 2.99 (s, 3H), 2.82- | | |
| | hydroxy-1-methylpyrrolidin-2-one | 2.72 (m, 1H), 2.49- | | |
| | | 2.38 (m, 1H). MS | | |
| | | (ESI⁺): m/z = 450.3. | | |
| 427 | 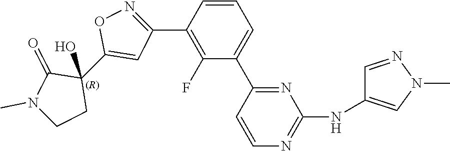 | ¹H NMR (500 MHz, Methanol-d₄) δ 8.48 (d, J = 5.3 Hz, 1H), 8.25- 8.13 (m, 1H), 8.11- 8.02 (m, 1H), 7.99 (d, J = 0.7 Hz, 1H), 7.66 (s, 1H), 7.49 (t, J = 7.8 Hz, 1H), 7.31-7.22 (m, 1H), 6.95 (d, J = 3.4 | RP HPLC (TFA modifier) | 10 |
| | (R)-3-(3-(2-Fluoro-3-(2-((1-methyl-1H-pyrazol-4-yl)amino)pyrimidin-4- | Hz, 1H), 3.91 (s, 3H), | | |
| | yl)phenyl)isoxazol-5-yl)-3-hydroxy-1-methylpyrrolidin-2-one | 3.67-3.54 (m, 2H), | | |
| | | 2.99 (s, 3H), 2.82- | | |
| | | 2.71 (m, 1H), 2.50- | | |
| | | 2.37 (m, 1H). | | |
| | | MS (ESI⁺): m/z = | | |
| | | 450.3. | | |

The compounds shown below in Table 9 were prepared in a manner analogous to (R)-3-hydroxy-1-methyl-3-(3-(6-(2-(oxetan-3-ylamino)pyrimidin-4-yl)pyridin-2-yl)isoxazol-5-yl)pyrrolidin-2-one (Example 66) using the appropriate amines in place of oxetan-3-amine.

TABLE 9

| | | | Purification | Yield |
|---|---|---|---|---|
| Ex. | Structure | Analytical data | conditions | (%) |
| 428 | 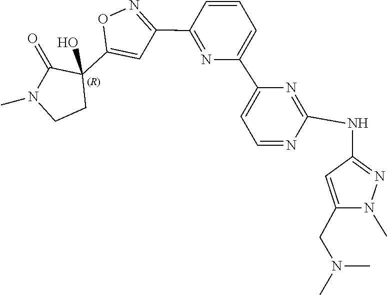 | ¹H NMR (500 MHz, Methanol-d₄) δ 8.61 (d, J = 5.1 Hz, 1H), 8.57 (dd, J = 7.9, 1.1 Hz, 1H), 8.17 (dd, J = 7.8, 1.1 Hz, 1H), 8.10 (t, J = 7.8 Hz, 1H), 7.93 (d, J = 5.1 Hz, 1H), 7.21 (s, 1H), 6.76 (s, 1H), 3.83 (s, 3H), 3.65-3.54 (m, 4H), 3.00 (s, 3H), 2.84- 2.76 (m, 1H), 2.52- 2.43 (m, 1H), 2.35 (s, 6H). MS (ESI⁺): m/z = 490.2 | RT HPLC (NH₄OH modifier) | 7 |
| | (R)-3-(3-(6-(2-((5- | | | |
| | ((Dimethylamino)methyl)-1- | | | |
| | methyl-1H-pyrazol-3- | | | |
| | yl)amino)pyrimidin-4-yl)pyridin- | | | |
| | 2-yl)isoxazol-5-yl)-3-hydroxy-1- | | | |
| | methylpyrrolidin-2-one | | | |
| 429 | 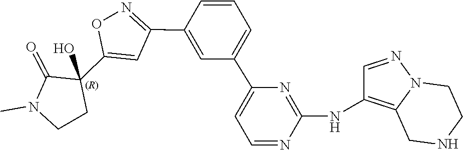 | ¹H NMR (500 MHz, Methanol-d₄) δ 8.55- 8.41 (m, 2H), 8.16 (dd, J = 7.8, 1.1 Hz, 1H), 8.07 (t, J = 7.8 Hz, 1H), 7.85 (d, J = 5.1 Hz, 1H), 7.70 (s, 1H), 7.20 (s, 1H), 4.16 (t, J = 5.6 Hz, 2H), 4.01 (s, 2H), 3.66-3.56 (m, 2H), | RT HPLC (NH₄OH modifier) | 72 |
| | (R)-3-Hydroxy-1-methyl-3-(3- | 3.00 (s, 3H), 2.83- | | |
| | (6-(2-((4,5,6,7- | 2.75 (m, 1H), 2.49- | | |
| | tetrahydropyrazolo[1,5- | 2.40 (m, 1H). MS | | |
| | a]pyrazin-3-yl)amino)pyrimidin- | (ESI⁺): m/z = 475.2. | | |
| | 4-yl)pyridin-2-yl)isoxazol-5- | | | |
| | yl)pyrrolidin-2-one | | | |
| 430 | 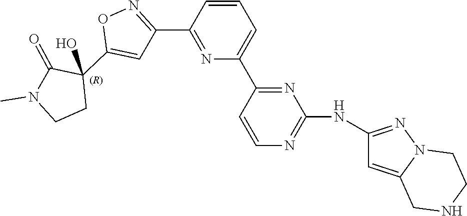 | ¹H NMR (500 MHz, Methanol-d₄) δ 8.66 (d, J = 5.2 Hz, 1H), 8.56 (dd, J = 7.7, 1.1 Hz, 1H), 8.20 (dd, J = 7.8, 1.1 Hz, 1H), 8.12 (t, J = 7.8 Hz, 1H), 7.98 (d, J = 5.2 Hz, 1H), 7.21 (s, 1H), 6.86 (s, 1H), 4.60 (s, 2H), 4.39 (t, J = 5.9 Hz, 2H), 3.90-3.83 (m, 2H), 3.67-3.56 (m, 2H), 3.00 (s, 3H), 2.83-2.75 (m, 1H), | RP HPLC (NH₄OH modifier) | 88 |
| | (R)-3-Hydroxy-1-methyl-3-(3- | 2.50-2.41 (m, 1H). | | |
| | (6-(2-((4,5,6,7- | MS (ESI⁺): m/z = | | |
| | tetrahydropyrazolo[1,5- | 475.2. | | |
| | a]pyrazin-2-yl)amino)pyrimidin- | | | |
| | 4-yl)pyridin-2-yl)isoxazol-5- | | | |
| | yl)pyrrolidin-2-one | | | |
| 431 | 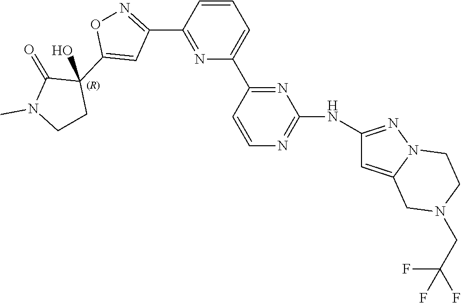 | ¹H NMR (500 MHz, Methanol-d₄) δ 8.61 (d, J = 5.1 Hz, 1H), 8.56 (dd, J = 7.7, 1.1 Hz, 1H), 8.17 (dd, J = 7.8, 1.1 Hz, 1H), 8.11 (t, J = 7.8 Hz, 1H), 7.93 (d, J = 5.1 Hz, 1H), 7.21 (s, 1H), 6.62 (s, 1H), 4.18- 4.08 (m, 2H), 4.02 (s, 2H), 3.65-3.55 (m, 2H), 3.42-3.35 (m, 2H), 3.29-3.24 (m, 2H), 3.00 (s, 3H), 2.83- 2.75 (m, 1H), 2.51- 2.40 (m, 1H). MS (ESI⁺): m/z = 556.5. | RP HPLC (NH₄OH modifier) | 7 |
| | (R)-3-Hydroxy-1-methyl-3-(3- | | | |
| | (6-(2-((5-(2,2,2-trifluoroethyl)- | | | |
| | 4,5,6,7-tetrahydropyrazolo[1,5- | | | |
| | a]pyrazin-2-yl)amino)pyrimidin- | | | |
| | 4-yl)pyridin-2-yl)isoxazol-5- | | | |
| | yl)pyrrolidin-2-one | | | |
| 432 | 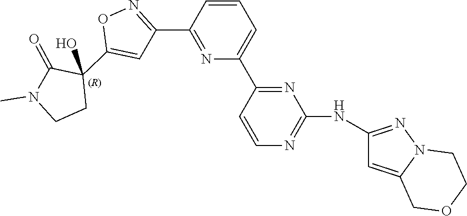 | ¹H NMR (500 MHz, Methanol-d₄) δ 8.71 (d, J = 5.5 Hz, 1H), 8.60 (dd, J = 7.9, 1.1 Hz, 1H), 8.25 (dd, J = 7.8, 1.0 Hz, 1H), 8.17 (t, J = 7.8 Hz, 1H), 8.08 (d, J = 5.6 Hz, 1H), 7.24 (s, 1H), 6.41 (s, 1H), 4.82 (s, 2H), 4.18 (dd, J = 7.1, 3.9 Hz, 4H), 3.66- 3.55 (m, 2H), 3.00 (s, 3H), 2.86-2.76 (m, 1H), 2.52-2.39 (m, | RP HPLC (NH₄OH modifier) | 6 |
| | (R)-3-(3-(6-(2-((6,7-Dihydro-4H- | 1H). MS (ESI⁺): m/z = | | |
| | pyrazolo[5,1-c][1,4]oxazin-2- | 475.4. | | |
| | yl)amino)pyrimidin-4-yl)pyridin- | | | |
| | 2-yl)isoxazol-5-yl)-3-hydroxy-1- | | | |
| | methylpyrrolidin-2-one | | | |
| 433 | 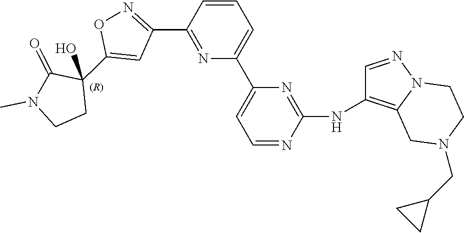 | ¹H NMR (500 MHz, Methanol-d₄) δ 8.61 (d, J = 5.1 Hz, 1H), 8.57 (dd, J = 7.8, 1.1 Hz, 1H), 8.20-8.13 (m, 1H), 8.11 (d, J = 7.8 Hz, 1H), 7.94 (d, J = 5.1 Hz, 1H), 7.21 (s, 1H), 6.64 (s, 1H), 4.14 (t, J = 5.7 Hz, 2H), 3.88 (s, 2H), 3.66-3.55 (m, 2H), 3.19-3.12 (m, 2H), 3.00 (s, 3H), 2.82- 2.73 (m, 1H), 2.55 (d, J = 6.7 Hz, 2H), 2.50- | RP HPLC (NH₄OH modifier) | 17 |
| | (R)-3-(3-(6-(2-((5- | 2.41 (m, 1H), 1.01 (d, J = | | |
| | (Cyclopropylmethyl)-4,5,6,7- | 8.0 Hz, 1H), 0.72- | | |
| | tetrahydropyrazolo[1,5- | 0.60 (m, 2H), 0.34- | | |
| | a]pyrazin-3-yl)amino)pyrimidin- | 0.21 (m, 2H). MS | | |
| | 4-yl)pyridin-2-yl)isoxazol-5-yl)- | (ESI⁺): m/z = 528.6. | | |
| | 3-hydroxy-1-methylpyrrolidin-2- | | | |
| | one | | | |
| 434 | 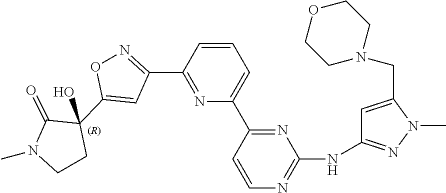 | ¹H NMR (500 MHz, Methanol-d₄) δ 8.65 (dd, J = 5.2, 1.2 Hz, 1H), 8.57 (dd, J = 7.8, 1.1 Hz, 1H), 8.20 (dd, J = 7.8, 1.1 Hz, 1H), 8.13 (dd, J = 8.1, 7.5 Hz, 1H), 7.97 (dd, J = 5.3, 3.0 Hz, 1H), 7.22 (s, 1H), 7.10 (s, 1H), 4.56 (s, 2H), 4.11-3.80 (m, 7H), 3.67-3.55 (m, | RT HPLC (TFA modifier) | 17 |
| | (R)-3-Hydroxy-1-methyl-3-(3- | 2H), 3.37 (d, J = 1.6 | | |
| | (6-(2-((1-methyl-5- | Hz, 4H), 3.00 (s, 3H), | | |
| | (morpholinomethyl)-1H-pyrazol- | 2.86-2.76 (m, 1H), | | |
| | 3-yl)amino)pyrimidin-4- | 2.51-2.41 (m, 1H). | | |
| | yl)pyridin-2-yl)isoxazol-5- | MS (ESI⁺): m/z = | | |
| | yl)pyrrolidin-2-one | 532.4. | | |
| 435 | 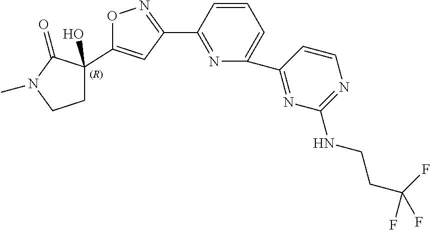 | ¹H NMR (500 MHz, Methanol-d₄) δ 8.52 (dd, J = 7.8, 1.1 Hz, 1H), 8.48 (d, J = 5.2 Hz, 1H), 8.15 (dd, J = 7.8, 1.1 Hz, 1H), 8.08 (t, J = 7.8 Hz, 1H), 7.79 (d, J = 5.1 Hz, 1H), 7.19 (s, 1H), 3.77 (t, J = 7.2 Hz, 2H), 3.65- 3.54 (m, 2H), 3.00 (s, 3H), 2.86-2.74 (m, 1H), 2.66-2.55 (m, 2H), 2.50-2.36 (m, | Solid product isolated through filtration. | 98 |
| | (R)-3-Hydroxy-1-methyl-3-(3- | 1H). MS (ESI⁺): m/z = | | |
| | (6-(2-((3,3,3- | 449.2. | | |
| | trifluoropropyl)amino)pyrimidin- | | | |
| | 4-yl)pyridin-2-yl)isoxazol-5- | | | |
| | yl)pyrrolidin-2-one | | | |
| 436 | 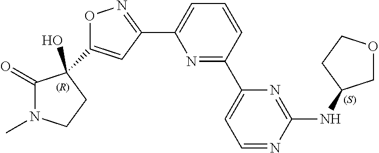 | ¹H NMR (500 MHz, Methanol-d₄) δ 8.52 (dd, J = 7.9, 1.1 Hz, 1H), 8.46 (d, J = 5.1 Hz, 1H), 8.14 (dd, J = 7.8, 1.1 Hz, 1H), 8.06 (t, J = 7.8 Hz, 1H), 7.76 (d, J = 5.1 Hz, 1H), 7.19 (s, 1H), 4.71- 4.62 (m, 1H), 4.14- | Solid product isolated through filtration. | 84 |
| | (R)-3-Hydroxy-1-methyl-3-(3- | 3.97 (m, 2H), 3.95- | | |
| | (6-(2-(((S)-tetrahydrofuran-3- | 3.88 (m, 1H), 3.78 (dd, | | |
| | yl)amino)pyrimidin-4-yl)pyridin- | J = 9.0, 3.9 Hz, 1H), | | |
| | 2-yl)isoxazol-5-yl)pyrrolidin-2- | 3.68-3.55 (m, 2H), | | |
| | one | 2.99 (s, 3H), 2.86- | | |
| | | 2.76 (m, 1H), 2.52- | | |
| | | 2.33 (m, 2H), 2.11- | | |
| | | 1.99 (m, 1H). | | |
| | | MS (ESI⁺): m/z = | | |
| | | 423.2. | | |
| 437 | 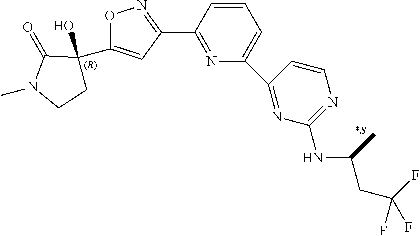 | ¹H NMR (500 MHz, Methanol-d₄) δ 8.40 (dd, J = 7.8, 1.1 Hz, 1H), 8.35 (d, J = 5.1 Hz, 1H), 8.02 (dd, J = 7.8, 1.1 Hz, 1H), 7.95 (t, J = 7.8 Hz, 1H), 7.64 (d, J = 5.1 Hz, 1H), 7.07 (s, 1H), 4.54- 4.41 (m, 1H), 3.54- 3.42 (m, 2H), 2.88 (s, 3H), 2.72-2.64 (m, 1H), 2.64-2.49 (m, 1H), 2.38-2.26 (m, 2H), 1.30 (d, J = 6.7 | Purified with SFC: the first eluting peak | 28 |
| | (R)-3-Hydroxy-1-methyl-3-(3- | Hz, 3H), 1.05 (d, J = | | |
| | (6-(2-(((*S)-4,4,4-trifluorobutan- | 6.2 Hz, 1H). MS (ESI⁺): | | |
| | 2-yl)amino)pyrimidin-4- | m/z = 463.2. | | |
| | yl)pyridin-2-yl)isoxazol-5- | | | |
| | yl)pyrrolidin-2-one | | | |
| 438 | 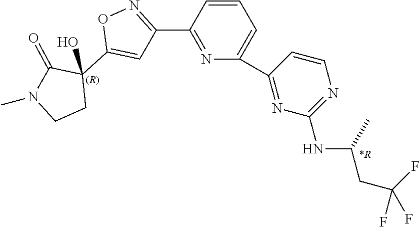 | ¹H NMR (500 MHz, Methanol-d₄) δ 8.52 (dd, J = 7.8, 1.1 Hz, 1H), 8.47 (d, J = 5.1 Hz, 1H), 8.15 (dd, J = 7.8, 1.1 Hz, 1H), 8.07 (t, J = 7.8 Hz, 1H), 7.77 (d, J = 5.1 Hz, 1H), 7.19 (s, 1H), 4.66- 4.55 (m, 1H), 3.67- 3.55 (m, 2H), 2.99 (s, 3H), 2.85-2.76 (m, 1H), 2.75-2.64 (m, 1H), 2.49-2.38 (m, | Purified with SFC: the second eluting peak | 39 |
| | (R)-3-Hydroxy-1-methyl-3-(3- | 2H), 1.42 (d, J = 6.7 | | |
| | (6-(2-(((*R)-4,4,4-trifluorobutan- | Hz, 3H), 1.17 (d, J = | | |
| | 2-yl)amino)pyrimidin-4- | 6.2 Hz, 1H). MS (ESI⁺): | | |
| | yl)pyridin-2-yl)isoxazol-5- | m/z = 463.2. | | |
| | yl)pyrrolidin-2-one | | | |
| 439 | 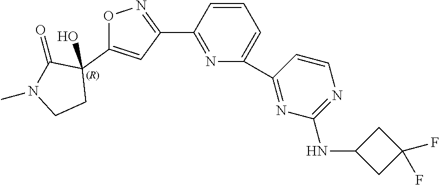 | ¹H NMR (500 MHz, Methanol-d₄) δ 8.52 (dd, J = 7.8, 1.1 Hz, 1H), 8.47 (d, J = 5.1 Hz, 1H), 8.14 (dd, J = 7.8, 1.1 Hz, 1H), 8.07 (t, J = 7.8 Hz, 1H), 7.79 (d, J = 5.1 Hz, 1H), 7.19 (s, 1H), 4.47- 4.33 (m, 1H), 3.66- 3.55 (m, 2H), 3.14- 3.04 (m, 2H), 2.99 (s, | Solid product isolated through filtration. | 89 |
| | (R)-3-(3-(6-(2-((3,3- | 3H), 2.84-2.74 (m, | | |
| | Difluorocyclobutyl)amino)pyrimi- | 1H), 2.73-2.64 (m, | | |
| | din-4-yl)pyridin-2-yl)isoxazol-5- | 2H), 2.50-2.38 (m, | | |
| | yl)-3-hydroxy-1- | 1H). | | |
| | methylpyrrolidin-2-one | MS (ESI⁺): m/z = | | |
| | | 443.1. | | |
| 440 | 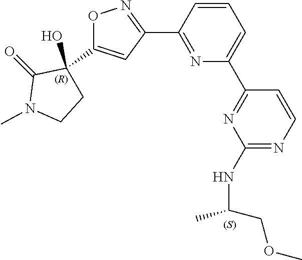 | ¹H NMR (500 MHz, Methanol-d₄) δ 8.52 (dd, J = 7.8, 1.1 Hz, 1H), 8.44 (d, J = 5.1 Hz, 1H), 8.15 (dd, J = 7.8, 1.1 Hz, 1H), 8.07 (t, J = 7.8 Hz, 1H), 7.73 (d, J = 5.1 Hz, 1H), 7.19 (s, 1H), 4.40 (d, J = 5.3 Hz, 1H), 3.66- 3.53 (m, 3H), 3.53- 3.45 (m, 1H), 3.42 (s, 3H), 2.99 (s, 3H), 2.85- 2.74 (m, 1H), 2.52- 2.37 (m, 1H), 1.32 (d, J = 6.7 Hz, 3H). MS | RT HPLC (NH₄OH modifier) | 58 |
| | (R)-3-Hydroxy-3-(3-(6-(2-(((S)- | (ESI⁺): m/z = 452.2. | | |
| | 1-methoxypropan-2- | | | |
| | yl)amino)pyrimidin-4-yl)pyridin- | | | |
| | 2-yl)isoxazol-5-yl)-1- | | | |
| | methylpyrrolidin-2-one | | | |
| 441 | 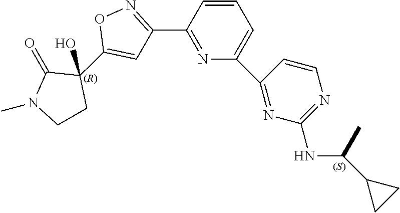 | ¹H NMR (500 MHz, Methanol-d₄) δ 8.49 (dd, J = 7.8, 1.1 Hz, 1H), 8.41 (d, J = 5.1 Hz, 1H), 8.14 (dd, J = 7.8, 1.1 Hz, 1H), 8.06 (t, J = 7.8 Hz, 1H), 7.69 (d, J = 5.1 Hz, 1H), 7.19 (s, 1H), 3.71- 3.57 (m, 3H), 2.99 (s, 3H), 2.83-2.73 (m, 1H), 2.68 (s, 2H), 2.51- 2.39 (m, 1H), 1.36 (d, | Solid product isolated through filtration. | 82 |
| | (R)-3-(3-(6-(2-(((S)-1- | J = 6.6 Hz, 3H), 1.13- | | |
| | Cyclopropylethyl)amino)pyrimidin- | 1.01 (m, 1H), 0.66- | | |
| | 4-yl)pyridin-2-yl)isoxazol-5- | 0.41 (m, 3H), 0.36- | | |
| | yl)-3-hydroxy-1- | 0.19 (m, 1H). MS | | |
| | methylpyrrolidin-2-one | (ESI⁺): m/z = 421.2. | | |
| 442 | 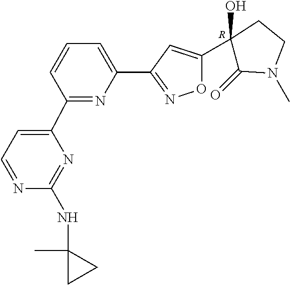 | ¹H NMR (500 MHz, Methanol-d₄) δ 8.57 (d, J = 7.8 Hz, 1H), 8.46 (d, J = 5.2 Hz, 1H), 8.15 (dd, J = 7.8, 1.1 Hz, 1H), 8.08 (t, J = 7.8 Hz, 1H), 7.77 (d, J = 5.2 Hz, 1H), 7.20 (s, 1H), 4.59 (s, 1H), 3.69- 3.55 (m, 2H), 3.00 (s, 3H), 2.84-2.75 (m, 1H), 2.52-2.38 (m, 1H), 1.52 (s, 3H), 0.92- 0.83 (m, 2H), 0.83- 0.75 (m, 2H). MS (ESI⁺): m/z = 407.2. | RP HPLC (NH₄OH modifier) | 11 |
| | (R)-3-Hydroxy-1-methyl-3-(3- | | | |
| | (6-(2-((1- | | | |
| | methylcyclopropyl)amino)pyrimi- | | | |
| | din-4-yl)pyridin-2-yl)isoxazol-5- | | | |
| | yl)pyrrolidin-2-one | | | |
| 443 | 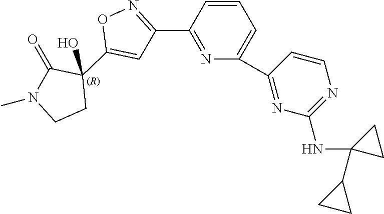 | ¹H NMR (500 MHz, Methanol-d₄) δ 8.57 (d, J = 7.7 Hz, 1H), 8.45 (d, J = 5.2 Hz, 1H), 8.15 (dd, J = 7.8, 1.1 Hz, 1H), 8.08 (t, J = 7.8 Hz, 1H), 7.77 (d, J = 5.1 Hz, 1H), 7.20 (s, 1H), 4.65-4.54 (m, 1H), 3.67-3.56 (m, 2H), 3.00 (s, 3H), 2.84- 2.73 (m, 1H), 2.50- 2.40 (m, 1H), 1.68- | RP HPLC (NH₄OH modifier) | 12 |
| | (R)-3-(3-(6-(2-([1,1′- | 1.55 (m, 1H), 0.87- | | |
| | Bi(cyclopropan)]-1- | 0.69 (m, 4H), 0.52- | | |
| | ylamino)pyrimidin-4-yl)pyridin- | 0.39 (m, 2H), 0.34- | | |
| | 2-yl)isoxazol-5-yl)-3-hydroxy-1- | 0.25 (m, 2H). MS | | |
| | methylpyrrolidin-2-one | (ESI⁺): m/z = 503.2. | | |
| 444 | 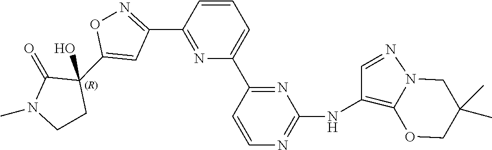 | ¹H NMR (500 MHz, Methanol-d₄) δ 8.58- 8.40 (m, 2H), 8.19 (d, J = 7.7 Hz, 1H), 8.09 (t, J = 7.8 Hz, 1H), 7.93 (d, J = 5.5 Hz, 1H), 7.61 (s, 1H), 7.21 (s, 1H), 4.04 (s, 2H), 3.91 (s, 2H), 3.68-3.56 (m, 2H), 3.00 (s, 3H), 2.87- | RP HPLC (TFA modifier) | 4 |
| | (R)-3-(3-(6-(2-((6,6-Dimethyl- | 2.74 (m, 1H), 2.52- | | |
| | 6,7-dihydro-5H-pyrazolo[5,1- | 2.38 (m, 1H), 1.19 (s, | | |
| | b][1,3]oxazin-3- | 6H). MS (ESI⁺): m/z = | | |
| | yl)amino)pyrimidin-4-yl)pyridin- | 503.2. | | |
| | 2-yl)isoxazol-5-yl)-3-hydroxy-1- | | | |
| | methylpyrrolidin-2-one | | | |
| 445 | 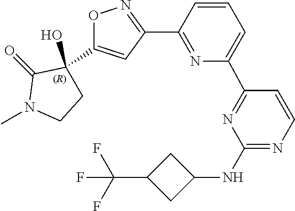 | ¹H NMR (500 MHz, Methanol-d₄) δ 8.36 (dd, J = 7.8, 1.1 Hz, 1H), 8.33 (d, J = 5.2 Hz, 1H), 8.02 (dd, J = 7.8, 1.1 Hz, 1H), 7.95 (t, J = 7.8 Hz, 1H), 7.63 (d, J = 5.1 Hz, 1H), 7.06 (s, 1H), 4.57 (t, J = 7.8 Hz, 1H), 4.52- 4.40 (m, 1H), 3.52- 3.42 (m, 2H), 3.00- 2.91 (m, 1H), 2.88 (s, | Solid product isolated through filtration. | 87 |
| | (R)-3-Hydroxy-1-methyl-3-(3- | 4H), 2.71-2.63 (m, | | |
| | (6-(2-((3- | 1H), 2.55-2.50 (m, | | |
| | (trifluoromethyl)cyclobutyl)amino) | 1H), 2.36-2.28 (m, | | |
| | pyrimidin-4-yl)pyridin-2- | 3H). MS (ESI⁺): m/z = | | |
| | yl)isoxazol-5-yl)pyrrolidin-2-one | 475.1. | | |
| 446 | 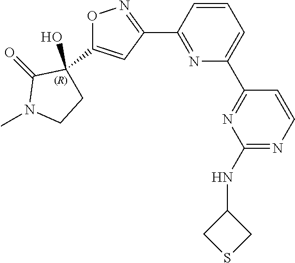 | ¹H NMR (500 MHz, Methanol-d₄) δ 8.44 (s, J = 7.7 Hz, 1H), 8.35 (d, J = 5.1 Hz, 1H), 8.03 (dd, J = 7.8, 1.1 Hz, 1H), 7.96 (t, J = 7.8 Hz, 1H), 7.66 (d, J = 5.1 Hz, 1H), 7.07 (s, 1H), 5.43-5.19 (m, 1H), 3.58-3.41 (m, 4H), 3.35-3.26 (m, 2H), 2.87 (s, 3H), 2.72- 2.61 (m, 1H), 2.38- 2.27 (m, 1H). MS (ESI⁺): m/z = 425.1. | RP HPLC (TFA modifier) | 30 |
| | (R)-3-Hydroxy-1-methyl-3-(3- | | | |
| | (6-(2-(thietan-3- | | | |
| | ylamino)pyrimidin-4-yl)pyridin- | | | |
| | 2-yl)isoxaozl-5-yl)pyrrolidin-2- | | | |
| | one | | | |
| 447 | 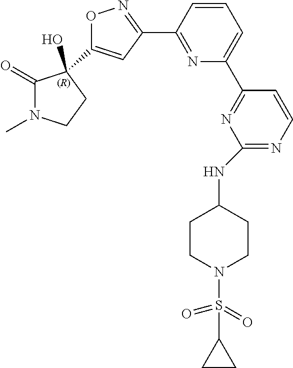 | ¹H NMR (500 MHz, Methanol-d₄) δ 8.56 (d, J = 7.8 Hz, 1H), 8.48 (d, J = 5.6 Hz, 1H), 8.22 (dd, J = 7.8, 1.1 Hz, 1H), 8.12 (t, J = 7.8 Hz, 1H), 7.89 (d, J = 5.6 Hz, 1H), 7.21 (s, 1H), 4.17 (s, 1H), 3.83 (d, J = 12.6 Hz, 2H), 3.68-3.53 (m, 2H), 3.15 (d, J = 11.5 Hz, 2H), 3.00 (s, 3H), 2.83- 2.74 (m, 1H), 2.59- 2.51 (m, 1H), 2.48- 2.40 (m, 1H), 2.21 (d, J = 12.0 Hz, 2H), 1.83- 1.72 (m, 2H), 1.16- 1.04 (m, 4H). MS (ESI⁺): m/z = 540.2. | RP HPLC (TFA modifier) | 28 |
| | (R)-3-(3-(6-(2-((1- | | | |
| | (Cyclopropylsulfonyl)piperidin- | | | |
| | 4-yl)amino)pyrimidin-4- | | | |
| | yl)pyridin-2-yl)isoxazol-5-yl)-3- | | | |
| | hydroxy-1-methylpyrrolidin-2- | | | |
| | one | | | |
| 448 | 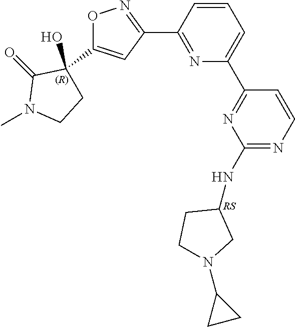 | ¹H NMR (500 MHz, Methanol-d₄) δ 8.53 (d, J = 7.7 Hz, 1H), 8.45 (d, J = 5.1 Hz, 1H), 8.15 (dd, J = 7.8, 1.1 Hz, 1H), 8.08 (t, J = 7.8 Hz, 1H), 7.76 (d, J = 5.1 Hz, 1H), 7.19 (s, 1H), 4.63 (s, 1H), 3.67- 3.57 (m, 2H), 3.23- 3.16 (m, 1H), 2.99 (s, 4H), 2.85-2.71 (m, 3H), 2.48-2.37 (m, 2H), 1.87-1.80 (m, 2H), 0.56-0.45 (m, 4H). MS (ESI⁺): m/z = 462.1. | RP HPLC (NH₄OH modifier) | 16 |
| | (3R)-3-(3-(6-(2-((1- | | | |
| | Cyclopropylpyrrolidin-3- | | | |
| | yl)amino)pyrimidin-4-yl)pyridin- | | | |
| | 2-yl)isoxazol-5-yl)-3-hydroxy-1- | | | |
| | methylpyrrolidin-2-one | | | |
| 449 | 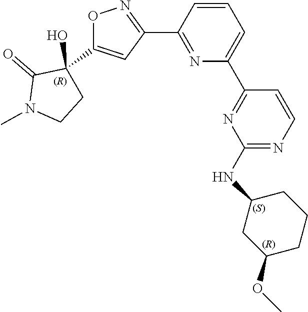 | ¹H NMR (500 MHz, Methanol-d₄) δ 8.51 (dd, J = 7.8, 1.1 Hz, 1H), 8.43 (d, J = 5.1 Hz, 1H), 8.15 (dd, J = 7.8, 1.1 Hz, 1H), 8.07 (t, J = 7.8 Hz, 1H), 7.72 (d, J = 5.1 Hz, 1H), 7.19 (s, 1H), 4.04 (s, 1H), 3.67-3.53 (m, 2H), 3.42 (s, 4H), 2.99 (s, 3H), 2.86-2.74 (m, 1H), 2.51-2.38 (m, 2H), 2.04 (s, 2H), 1.98- 1.85 (m, 1H), 1.52- 1.26 (m, 4H). MS (ESI⁺): m/z = 462.1. | RP HPLC (NH₄OH modifier) | 36 |
| | (R)-3-Hydroxy-3-(3-(6-(2- | | | |
| | (((1S,3R)-3- | | | |
| | methoxycyclohexyl)amino)pyri- | | | |
| | midin-4-yl)pyridin-2-yl)isoxazol- | | | |
| | 5-yl)-1-methylpyrrolidin-2-one | | | |
| 450 | 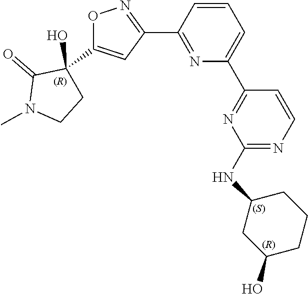 | ¹H NMR (500 MHz, Methanol-d₄) δ 8.50 (dd, J = 7.8, 1.1 Hz, 1H), 8.43 (d, J = 5.1 Hz, 1H), 8.14 (dd, J = 7.8, 1.1 Hz, 1H), 8.07 (t, J = 7.8 Hz, 1H), 7.71 (d, J = 5.2 Hz, 1H), 7.19 (s, 1H), 4.00 (s, 1H), 3.80-3.72 (m, 1H), 3.67-3.57 (m, 2H), 2.99 (s, 3H), 2.83- 2.74 (m, 1H), 2.52- 2.42 (m, 1H), 2.42- 2.34 (m, 1H), 2.01 (dd, J = 30.2, 12.1 Hz, 2H), 1.95-1.82 (m, 1H), 1.47 (q, J = 12.9 Hz, | RP HPLC (NH₄OH modifier) | 27 |
| | (R)-3-Hydroxy-3-(3-(6-(2- | 1H), 1.40-1.25 (m, | | |
| | (((1S,3R)-3- | 3H). MS (ESI⁺): m/z = | | |
| | hydroxycyclohexyl)amino)pyrimi- | 451.2. | | |
| | din-4-yl)pyridin-2-yl)isoxazol-5- | | | |
| | yl)-1-methylpyrrolidin-2-one | | | |
| 451 | 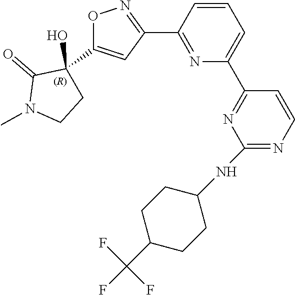 | ¹H NMR (500 MHz, Methanol-d₄) δ 8.56- 8.49 (m, 1H), 8.49- 8.39 (m, 1H), 8.18- 8.11 (m, 1H), 8.11- 8.04 (m, 1H), 7.79- 7.70 (m, 1H), 7.19 (d, J = 2.0 Hz, 1H), 3.67- 3.55 (m, 2H), 2.99 (s, 3H), 2.78 (ddd, J = 13.5, 7.1, 4.8 Hz, 1H), 2.49-2.42 (m, 1H), 2.31-2.21 (m, 1H), 2.16-2.09 (m, 1H), 1.92-1.68 (m, 6H). MS (ESI⁺): m/z = 503.2. | Solid product isolated through filtration | 85 |
| | (R)-3-Hydroxy-1-methyl-3-(3- | | | |
| | (6-(2-((4- | | | |
| | (trifluoromethyl)cyclohexyl)amino) | | | |
| | pyrimidin-4-yl)pyridin-2- | | | |
| | yl)isoxazol-5-yl)pyrrolidin-2-one | | | |
| 452 | 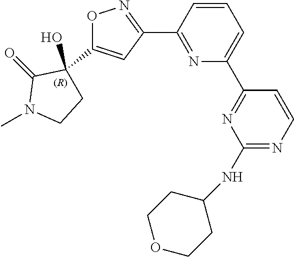 | ¹H NMR (500 MHz, Methanol-d₄) δ 8.59- 8.51 (m, 1H), 8.45 (d, J = 5.1 Hz, 1H), 8.15 (dd, J = 7.8, 1.1 Hz, 1H), 8.07 (t, J = 7.8 Hz, 1H), 7.73 (d, J = 5.1 Hz, 1H), 7.19 (s, 1H), 4.17 (s, 1H), 4.09-3.94 (m, 2H), 3.68-3.57 (m, 4H), 2.99 (s, 3H), 2.84- 2.75 (m, 1H), 2.52- 2.40 (m, 1H), 2.07 (d, J = 13.6 Hz, 2H), 1.75- 1.61 (m, 2H). | Solid product isolated through filtration | 65 |
| | (R)-3-Hydroxy-1-methyl-3-(3- | | | |
| | (6-(2-((tetrahydro-2H-pyran-4- | | | |
| | yl)amino)pyrimidin-4-yl)pyridin- | | | |
| | 2-yl)isoxazol-5-yl)pyrrolidin-2- | | | |
| | one | | | |
| 453 | 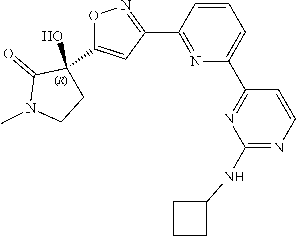 | ¹H NMR (500 MHz, Methanol-d₄) δ 8.56- 8.49 (m, 1H), 8.49- 8.39 (m, 1H), 8.18- 8.11 (m, 1H), 8.11- 8.04 (m, 1H), 7.79- 7.70 (m, 1H), 7.19 (d, J = 2.0 Hz, 1H), 3.67- 3.55 (m, 2H), 2.99 (s, 3H), 2.78 (ddd, J = 13.5, 7.1, 4.8 Hz, 1H), 2.49-2.42 (m, 1H), 2.31-2.21 (m, 1H), 2.16-2.09 (m, 1H), 1.92-1.68 (m, 6H). | Solid product isolated through filtration | 97 |
| | (R)-3-(3-(6-(2- | MS (ESI⁺): m/z = | | |
| | (Cyclobutylamino)pyrimidin-4- | 407.2. | | |
| | yl)pyridin-2-yl)isoxazol-5-yl)-3- | | | |
| | hydroxy-1-methylpyrrolidin-2- | | | |
| | one | | | |
| 454 | 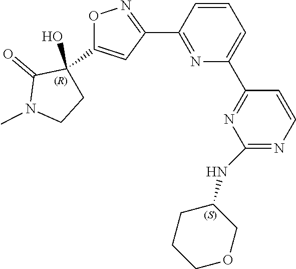 | ¹H NMR (500 MHz, Methanol-d₄) δ 8.41 (d, J = 7.8 Hz, 1H), 8.35 (d, J = 5.1 Hz, 1H), 8.04 (d, J = 7.8, 1.0 Hz, 1H), 7.97 (t, J = 7.8, 0.9 Hz, 1H), 7.64 (d, J = 5.2, 1.1 Hz, 1H), 7.09 (s, 1H), 4.10-3.93 (m, 2H), 3.82-3.71 (m, 1H), 3.57-3.44 (m, 3H), 3.33-3.27 (m, 1H), 2.90 (s, 4H), 2.76- 2.67 (m, 1H), 2.42- 2.31 (m, 1H), 2.10- 2.00 (m, 1H), 1.81- 1.73 (m, 1H), 1.73- | RP HPLC (NH₄OH modifier) | 80 |
| | (R)-3-Hydroxy-1-methyl-3-(3- | 1.61 (m, 2H). MS | | |
| | (6-(2-(((S)-tetrahydro-2H-pyran- | (ESI⁺): m/z = 437.1. | | |
| | 3-yl)amino)pyrimidin-4- | | | |
| | yl)pyridin-2-yl)isoxaozl-5- | | | |
| | yl)pyrrolidin-2-one | | | |
| 455 | 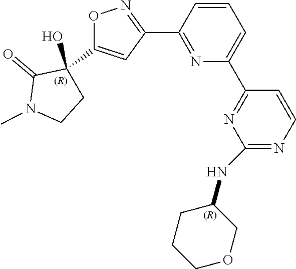 | ¹H NMR (500 MHz, Methanol-d₄) δ 8.41 (d, J = 7.8 Hz, 1H), 8.35 (d, J = 5.1 Hz, 1H), 8.04 (d, J = 7.8, 1.0 Hz, 1H), 7.97 (t, J = 7.8, 0.9 Hz, 1H), 7.64 (d, J = 5.2, 1.1 Hz, 1H), 7.09 (s, 1H), 4.10-3.93 (m, 2H), 3.82-3.71 (m, 1H), 3.57-3.44 (m, 3H), 3.33-3.27 (m, 1H), 2.90 (s, 4H), 2.76- 2.67 (m, 1H), 2.42- 2.31 (m, 1H), 2.10- 2.00 (m, 1H), 1.81- 1.73 (m, 1H), 1.73- | RP HPLC (NH₄OH modifier) | 80 |
| | (R)-3-Hydroxy-1-methyl-3-(3- | 1.61 (m, 2H). MS | | |
| | (6-(2-(((R)-tetrahydro-2H- | (ESI⁺): m/z = 437.1. | | |
| | pyran-3-yl)amino)pyrimidin-4- | | | |
| | yl)pyridin-2-yl)isoxazol-5- | | | |
| | yl)pyrrolidin-2-one | | | |
| 456 | 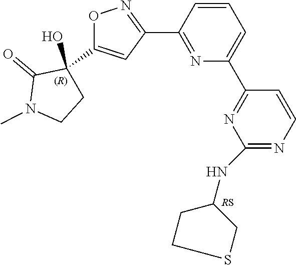 | ¹H NMR (500 MHz, Methanol-d₄) δ 8.53 (dd, J = 7.8, 1.1 Hz, 1H), 8.47 (d, J = 5.1 Hz, 1H), 8.15 (dd, J = 7.9, 1.1 Hz, 1H), 8.07 (t, J = 7.8 Hz, 1H), 7.77 (d, J = 5.1 Hz, 1H), 7.19 (s, 1H), 4.82- 4.72 (m, 1H), 3.66- 3.50 (m, 2H), 3.24 (dd, J = 10.8, 5.7 Hz, 1H), 3.04-2.95 (m, 5H), 2.85 (dd, J = 10.8, 5.5 Hz, 1H), 2.83-2.75 (m, 1H), 2.49-2.39 | RP HPLC (NH₄OH modifier) | 42 |
| | (3R)-3-Hydroxy-1-methyl-3-(3- | (m, 1H), 2.32-2.14 | | |
| | (6-(2-((tetrahydrothiophen-3- | (m, 2H). MS (ESI⁺): | | |
| | yl)amino)pyrimidin-4-yl)pyridin- | m/z = 439.1. | | |
| | 2-yl)isoxazol-5-yl)pyrrolidin-2- | | | |
| | one | | | |
| 457 | 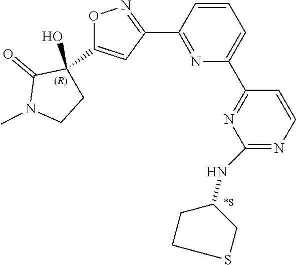 | ¹H NMR (500 MHz, Methanol-d₄) δ 8.41 (dd, J = 7.8, 1.1 Hz, 1H), 8.35 (d, J = 5.1 Hz, 1H), 8.03 (dd, J = 7.8, 1.1 Hz, 1H), 7.95 (t, J = 7.8 Hz, 1H), 7.64 (d, J = 5.1 Hz, 1H), 7.07 (s, 1H), 4.66 (s, 1H), 3.54-3.42 (m, 2H), 3.18-3.07 (m, 1H), 2.96-2.81 (m, 5H), 2.80-2.66 (m, 2H), 2.40-2.28 (m, 1H), 2.20-2.01 (m, 2H). | Example 456 was separated by SFC: The first eluting peak was designated Example 457 and the second eluting peak was designated Example 458. | 17 |
| | (R)-3-Hydroxy-1-methyl-3-(3- | MS (ESI⁺): m/z = | | |
| | (6-(2-(((*S)-tetrahydrothiophen- | 439.1. | | |
| | 3-yl)amino)pyrimidin-4- | | | |
| | yl)pyridin-2-yl)isoxazol-5- | | | |
| | yl)pyrrolidin-2-one | | | |
| 458 | 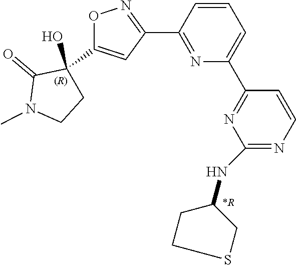 | ¹H NMR (500 MHz, Methanol-d₄) δ 8.53 (dd, J = 7.8, 1.1 Hz, 1H), 8.47 (d, J = 5.1 Hz, 1H), 8.14 (dd, J = 7.8, 1.1 Hz, 1H), 8.07 (t, J = 7.8 Hz, 1H), 7.76 (d, J = 5.1 Hz, 1H), 7.19 (s, 1H), 4.77 (s, 1H), 3.68-3.53 (m, 2H), 3.24 (dd, J = 10.7, 5.6 Hz, 1H), 3.06- 2.94 (m, 5H), 2.92- 2.73 (m, 2H), 2.50- 2.39 (m, 1H), 2.32- 2.10 (m, 2H). MS (ESI⁺): m/z = 439.1. | Example 456 was separated by SFC: The first eluting peak was designated Example 457 and the second eluting peak was designated Example 458. | 17 |
| | (R)-3-Hydroxy-1-methyl-3-(3- | | | |
| | (6-(2-(((*R)-tetrahydrothiophen- | | | |
| | 3-yl)amino)pyrimidin-4- | | | |
| | yl)pyridin-2-yl)isoxazol-5- | | | |
| | yl)pyrrolidin-2-one | | | |
| 459 | 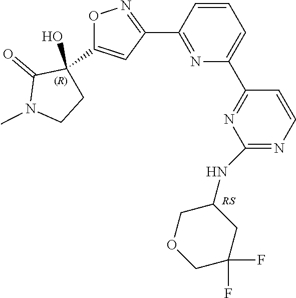 | ¹H NMR (500 MHz, Methanol-d₄) δ 8.56- 8.42 (m, 2H), 8.19- 8.11 (m, 1H), 8.08 (t, J = 7.8 Hz, 1H), 7.80 (d, J = 5.1 Hz, 1H), 7.19 (s, 1H), 4.51 (s, 1H), 4.08 (d, J = 11.3 Hz, 1H), 3.90-3.82 (m, 1H), 3.73-3.59 (m, 3H), 3.50 (d, J = 14.7 Hz, 1H), 2.99 (s, 3H), 2.84-2.76 (m, 1H), 2.61-2.52 (m, 1H), 2.52-2.40 (m, 1H), 2.26-2.12 (m, 1H). MS (ESI⁺): m/z = 473.2. | RP HPLC (TFA modifier) | 47 |
| | (3R)-3-(3-(6-(2-((5,5- | | | |
| | Difluorotetrahydro-2H-pyran-3- | | | |
| | yl)amino)pyrimidin-4-yl)pyridin- | | | |
| | 2-yl)isoxazol-5-yl)-3-hydroxy-1- | | | |
| | methylpyrrolidin-2-one | | | |
| 460 | 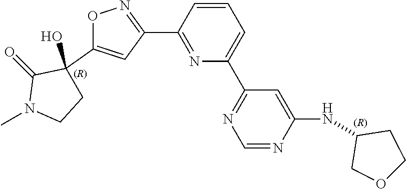 | ¹H NMR (500 MHz, Methanol-d₄) δ 8.53 (dd, J = 7.8, 1.1 Hz, 1H), 8.47 (d, J = 5.1 Hz, 1H), 8.15 (dd, J = 7.9, 1.1 Hz, 1H), 8.07 (t, J = 7.8 Hz, 1H), 7.77 (d, J = 5.1 Hz, 1H), 7.19 (s, 1H), 4.66 (s, 1H), 4.14-3.98 (m, 2H), 3.96-3.86 (m, 1H), 3.78 (dd, J = 9.0, | Solid product isolated through filtration | 72 |
| | (R)-3-Hydroxy-1-methyl-3-(3- | 3.9 Hz, 1H), 3.65- | | |
| | (6-(2-(((R)-tetrahydrofuran-3- | 3.55 (m, 2H), 2.99 (s, | | |
| | yl)amino)pyrimidin-4-yl)pyridin- | 3H), 2.84-2.73 (m, | | |
| | 2-yl)isoxazol-5-yl)pyrrolidin-2- | 1H), 2.51-2.32 (m, | | |
| | one | 2H), 2.10-1.95 (m, | | |
| | | 1H). MS (ESI⁺): m/z = | | |
| | | 423.1. | | |
| 461 | 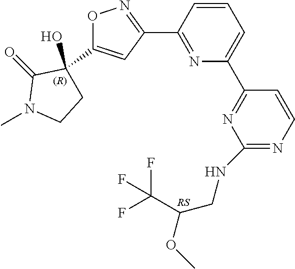 | ¹H NMR (400 MHz, Methanol-d₄) δ 8.60- 8.44 (m, 2H), 8.16 (dd, J = 7.8, 1.2 Hz, 1H), 8.08 (t, J = 7.8 Hz, 1H), 7.81 (d, J = 5.1 Hz, 1H), 7.20 (s, 1H), 4.13- 3.90 (m, 2H), 3.67- 3.53 (m, 6H), 3.00 (s, 3H), 2.83-2.71 (m, 1H), 2.48-2.38 (m, 1H). MS (ESI⁺): m/z = 479.1. | Solid product isolated through filtration | 47 |
| | (3R)-3-Hydroxy-1-methyl-3-(3- | | | |
| | (6-(2-((3,3,3-trifluoro-2- | | | |
| | methoxypropyl)amino)pyrimidin- | | | |
| | 4-yl)pyridin-2-yl)isoxazol-5- | | | |
| | yl)pyrrolidin-2-one | | | |
| 462 | 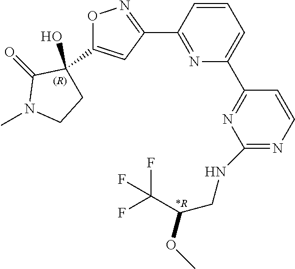 | ¹H NMR (500 MHz, Methanol-d₄) δ 8.56- 8.45 (m, 2H), 8.15 (dd, J = 7.8, 1.1 Hz, 1H), 8.08 (t, J = 7.8 Hz, 1H), 7.81 (d, J = 5.1 Hz, 1H), 7.20 (s, 1H), 4.12- 3.90 (m, 2H), 3.64- 3.55 (m, 6H), 3.00 (s, 3H), 2.84-2.76 (m, 1H), 2.51-2.36 (m, 1H). MS (ESI⁺): m/z = 479.1. | SFC of Example 461: The first eluting peak was designated Example 462 and the second eluting peak was designated Example 463. | 20 |
| | (R)-3-Hydroxy-1-methyl-3-(3- | | | |
| | (6-(2-(((*R)-3,3,3-trifluoro-2- | | | |
| | methoxypropyl)amino)pyrimidin- | | | |
| | 4-yl)pyridin-2-yl)isoxazol-5- | | | |
| | yl)pyrrolidin-2-one | | | |
| 463 | 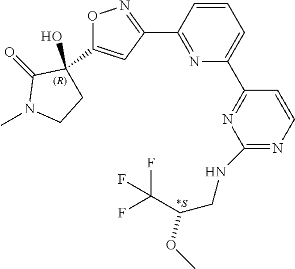 | ¹H NMR (500 MHz, Methanol-d₄) δ 8.57- 8.41 (m, 2H), 8.16 (dd, J = 7.8, 1.1 Hz, 1H), 8.08 (t, J = 7.8 Hz, 1H), 7.81 (d, J = 5.1 Hz, 1H), 7.20 (s, 1H), 4.13- 3.89 (m, 2H), 3.68- 3.51 (m, 6H), 3.00 (s, 3H), 2.85-2.74 (m, 1H), 2.52-2.34 (m, 1H). MS (ESI⁺): m/z = 479.1. | SFC of Example 461: The first eluting peak was designated Example 462 and the second eluting peak was designated Example 463. | 20 |
| | (R)-3-Hydroxy-1-methyl-3-(3- | | | |
| | (6-(2-(((*S)-3,3,3-trifluoro-2- | | | |
| | methoxypropyl)amino)pyrimidin- | | | |
| | 4-yl)pyridin-2-yl)isoxazol-5- | | | |
| | yl)pyrrolidin-2-one | | | |
| 464 | 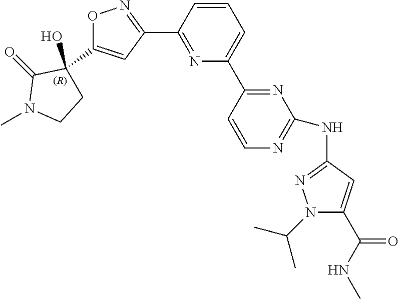 | ¹H NMR (500 MHz, Methanol-d₄) δ 8.66- 8.51 (m, 2H), 8.20- 8.12 (m, 1H), 8.09 (d, J = 7.8 Hz, 1H), 7.93 (d, J = 5.1 Hz, 1H), 7.20 (d, J = 7.9 Hz, 2H), 5.56-5.34 (m, 1H), 3.70-3.52 (m, 2H), 3.00 (s, 3H), 2.92 (s, 3H), 2.82-2.72 (m, 1H), 2.48-2.37 (m, 1H), 1.49 (d, J = 6.6 Hz, 6H). MS (ESI⁺): m/z = 518.3. | RP HPLC (NH₄OH modifier) and SFC | 6 |
| | (R)-3-((4-(6-(5-(3-Hydroxy-1- | | | |
| | methyl-2-oxopyrrolidin-3- | | | |
| | yl)isoxazol-3-yl)pyridin-2- | | | |
| | yl)pyrimidin-2-yl)amino)-1- | | | |
| | isopropyl-N-methyl-1H- | | | |
| | pyrazole-5-carboxamide | | | |
| 465 | 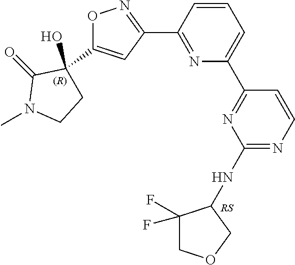 | ¹H NMR (500 MHz, Methanol-d₄) δ 8.55 (dd, J = 7.9, 1.1 Hz, 1H), 8.50 (d, J = 5.1 Hz, 1H), 8.13 (dd, J = 7.8, 1.1 Hz, 1H), 8.06 (t, J = 7.8 Hz, 1H), 7.83 (d, J = 5.1 Hz, 1H), 7.18 (s, 1H), 5.05 (dd, J = 14.7, 7.3 Hz, 1H), 4.50-4.38 (m, 1H), 4.20-4.09 (m, 1H), 3.96 (dt, J = 17.3, 11.3 Hz, 1H), 3.90-3.78 (m, 1H), 3.64-3.47 (m, 2H), 2.97 (s, 3H), | RP HPLC (NH₄OH modifier) | 15 |
| | (3R)-3-(3-(6-(2-((4,4- | 2.81-2.69 (m, 1H), | | |
| | Difluorotetrahydrofuran-3- | 2.48-2.33 (m, 1H). | | |
| | yl)amino)pyrimidin-4-yl)pyridin- | MS (ESI⁺): m/z = | | |
| | 2-yl)isoxazol-5-yl)-3-hydroxy-1- | 459.1. | | |
| | methylpyrrolidin-2-one | | | |
| 466 | 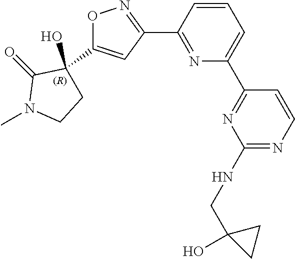 | ¹H NMR (500 MHz, Methanol-d₄) δ 8.49 (dd, J = 7.8, 1.1 Hz, 1H), 8.42 (d, J = 5.1 Hz, 1H), 8.13 (dd, J = 7.8, 1.1 Hz, 1H), 8.05 (t, J = 7.8 Hz, 1H), 7.71 (d, J = 5.1 Hz, 1H), 7.18 (s, 1H), 3.69 (s, 2H), 3.64-3.54 (m, 2H), 2.97 (s, 3H), 2.82- 2.73 (m, 1H), 2.47- 2.37 (m, 1H), 0.80- 0.68 (m, 4H). MS (ESI⁺): m/z = 423.2. | RP HPLC (NH₄OH modifier) | 4 |
| | (R)-3-Hydroxy-3-(3-(6-(2-(((1- | | | |
| | hydroxycyclopropyl)methyl)ami- | | | |
| | no)pyrimidin-4-yl)pyridin-2- | | | |
| | yl)isoxazol-5-yl)-1- | | | |
| | mehtylpyrrolidin-2-one | | | |
| 467 | 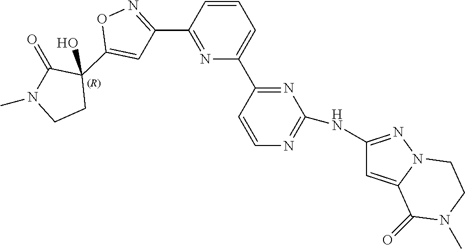 | ¹H NMR (500 MHz, Methanol-d₄) δ 8.66 (d, J = 5.3 Hz, 1H), 8.60 (dd, J = 7.8, 1.1 Hz, 1H), 8.19 (dd, J = 7.8, 1.1 Hz, 1H), 8.13 (t, J = 7.8 Hz, 1H), 8.01 (d, J = 5.3 Hz, 1H), 7.24 (s, 1H), 7.21 (s, 1H), 4.37 (dd, J = 7.3, 5.3 Hz, 2H), 3.89 (dd, J = 7.1, 5.5 Hz, 2H), 3.67- 3.53 (m, 2H), 3.15 (s, 3H), 2.98 (s, 3H), 2.82- 2.72 (m, 1H), 2.49- 2.37 (m, 1H). MS | RP HPLC (TFA modifier) | 17 |
| | (R)-2-((4-(6-(5-(3-Hydroxy-1- | (ESI⁺): m/z = 502.5. | | |
| | methyl-2-oxopyrrolidin-3- | | | |
| | yl)isoxazol-3-yl)pyridin-2- | | | |
| | yl)pyrimidin-2-yl)amino)-5- | | | |
| | methyl-6,7-dihydropyrazolo[1,5- | | | |
| | a]pyrazin-4(5H)-one | | | |
| 468 | 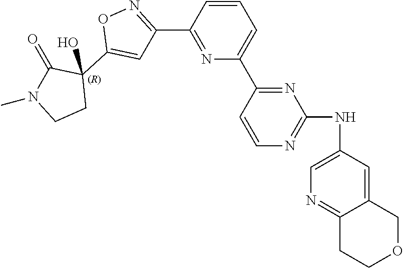 | ¹H NMR (400 MHz, Methanol-d₄) δ 9.39 (s, 1H), 8.75 (d, J = 5.1 Hz, 1H), 8.53 (d, J = 7.8 Hz, 1H), 8.31 (s, 1H), 8.19 (dd, J = 7.9, 1.1 Hz, 1H), 8.15- 8.06 (m, 2H), 7.20 (s, 1H), 4.91 (s, 2H), 4.12 (t, J = 5.7 Hz, 2H), 3.63- 3.51 (m, 2H), 3.11 (d, J = 5.8 Hz, 2H), 2.98 (s, 3H), 2.85-2.73 (m, 1H), 2.50-2.26 (m, 1H). MS (ESI⁺): m/z = 486.4. | RP HPLC (NH₄OH modifier) | 20 |
| | (R)-3-(3-(6-(2-((7,8-Dihydro-5H- | | | |
| | pyrano[4,3-b]pyridin-3- | | | |
| | yl)amino)pyrimidin-4-yl)pyridin- | | | |
| | 2-yl)isoxazol-5-yl)-3-hydroxy-1- | | | |
| | methylpyrrolidin-2-one | | | |
| 469 | 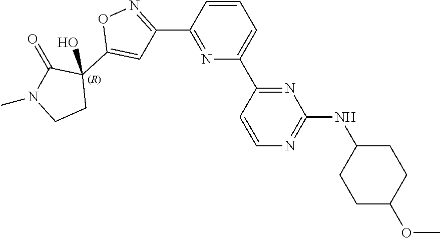 | ¹H NMR (500 MHz, Methanol-d₄) δ 8.47 (dd, J = 7.8, 1.2 Hz, 1H), 8.40 (d, J = 5.2 Hz, 1H), 8.12 (dd, J = 7.8, 1.1 Hz, 1H), 8.04 (t, J = 7.8 Hz, 1H), 7.68 (d, J = 5.1 Hz, 1H), 7.16 (s, 1H), 3.90 (s, 1H), 3.68-3.49 (m, 2H), 3.37 (s, 3H), 3.29- 3.18 (m, 1H), 2.97 (s, 3H), 2.80-2.70 (m, 1H), 2.49-2.33 (m, 1H), 2.23-2.03 (m, | RP HPLC (TFA modifier) | 35 |
| | (R)-3-Hydroxy-3-(3-(6-(2-((4- | 4H), 1.51-1.27 (m, | | |
| | methoxycyclohexyl)amino)pyri- | 4H). MS (ESI⁺): m/z = | | |
| | midin-4-yl)pyridin-2-yl)isoxazol- | 465.3. | | |
| | 5-yl)-1-methylpyrrolidin-2-one | | | |
| 470 | 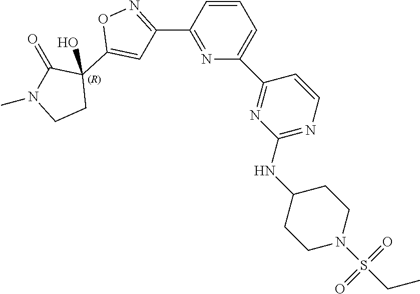 | ¹H NMR (500 MHz, Methanol-d₄) δ 8.51 (dd, J = 7.8, 1.1 Hz, 1H), 8.45 (d, J = 5.1 Hz, 1H), 8.15 (dd, J = 7.8, 1.1 Hz, 1H), 8.07 (t, J = 7.8 Hz, 1H), 7.74 (d, J = 5.1 Hz, 1H), 7.19 (s, 1H), 4.11 (s, 1H), 3.86-3.76 (m, 2H), 3.67-3.55 (m, 2H), 3.18-3.04 (m, 4H), 2.99 (s, 3H), 2.85- 2.75 (m, 1H), 2.51- 2.40 (m, 1H), 2.18 (dd, J = 13.2, 3.7 Hz, 2H), 1.75-1.59 (m, 2H), 1.37 (t, J = 7.4 Hz, 3H). | Solid product isolated through filtration | 44 |
| | (R)-3-(3-(6-(2-((1- | MS (ESI⁺): m/z = | | |
| | (Ethylsulfonyl)piperidin-4- | 528.2. | | |
| | yl)amino)pyrimidin-4-yl)pyridin- | | | |
| | 2-yl)isoxazol-5-yl)-3-hydroxy-1- | | | |
| | methylpyrrolidin-2-one | | | |
| 471 | 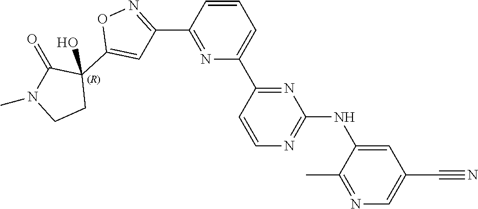 | ¹H NMR (500 MHz, Methanol-d₄) δ 8.80 (d, J = 1.9 Hz, 1H), 8.69 (d, J = 5.1 Hz, 1H), 8.55 (d, J = 1.9 Hz, 1H), 8.47 (dd, J = 7.8, 1.1 Hz, 1H), 8.19 (dd, J = 7.8, 1.1 Hz, 1H), 8.11 (t, J = 7.8 Hz, 1H), 8.06 (d, J = 5.1 Hz, 1H), 7.22 (s, 1H), 3.68- 3.59 (m, 2H), 3.00 (s, 3H), 2.86-2.76 (m, | RP HPLC (NH₄OH modifier) | 7 |
| | (R)-5-((4-(6-(5-(3-Hydroxy-1- | 1H), 2.70 (s, 3H), 2.50- | | |
| | methyl-2-oxopyrrolidin-3- | 2.38 (m, 1H). MS | | |
| | yl)isoxazol-3-yl)pyridin-2- | (ESI⁺): m/z = 469.2. | | |
| | yl)pyrimidin-2-yl)amino)-6- | | | |
| | methylnicotinonitrile | | | |
| 472 | 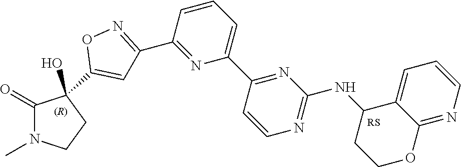 | ¹H NMR (500 MHz, Methanol-d₄) δ 8.61- 8.47 (m, 2H), 8.15 (dd, J = 7.8, 1.1 Hz, 1H), 8.10-7.98 (m, 2H), 7.89-7.76 (m, 2H), 7.20 (s, 1H), 7.02 (dd, J = 7.5, 4.9 Hz, 1H), 5.60-5.46 (m, 1H), 4.58-4.44 (m, 2H), 3.66-3.55 (m, 2H), | RP HPLC (NH₄OH modifier) | 17 |
| | (3R)-3-(3-(6-(2-((3,4-Dihydro- | 3.00 (s, 3H), 2.84- | | |
| | 2H-pyrano[2,3-b]pyridin-4- | 2.71 (m, 1H), 2.52- | | |
| | yl)amino)pyrimidin-4-yl)pyridin- | 2.41 (m, 1H), 2.40- | | |
| | 2-yl)isoxazol-5-yl)-3-hydroxy-1- | 2.24 (m, 2H). MS | | |
| | methylpyrrolidin-2-one | (ESI⁺): m/z = 487.2. | | |
| 473 | 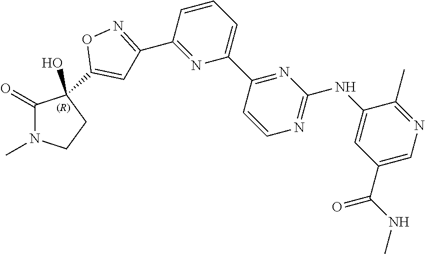 | ¹H NMR (500 MHz, Methanol-d₄) δ 9.37 (d, J = 12.8 Hz, 1H), 8.72 (dd, J = 3.6, 1.6 Hz, 2H), 8.56 (d, J = 7.8 Hz, 1H), 8.19 (d, J = 7.9 Hz, 1H), 8.12 (dd, J = 8.9, 6.6 Hz, 2H), 7.21 (dd, J = 2.7, 0.8 Hz, 1H), 3.68-3.52 (m, 2H), 3.07-2.97 (m, 6H), 2.84-2.75 (m, 4H), 2.51-2.40 (m, 1H). MS (ESI⁺): m/z = 501.5. | RP HPLC (TFA modifier) | 21 |
| | (R)-5-((4-(6-(5-(3-Hydroxy-1- | | | |
| | methyl-2-oxopyrrolidin-3- | | | |
| | yl)isoxazol-3-yl)pyridin-2- | | | |
| | yl)pyrimidin-2-yl)amino)-N,6- | | | |
| | dimethylnicotinamide | | | |
| 474 | 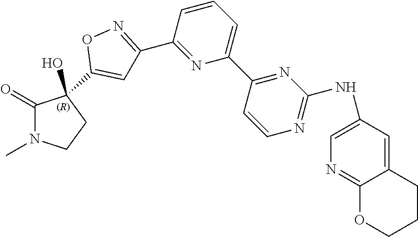 | ¹H NMR (500 MHz, Methanol-d₄) δ 8.81 (d, J = 2.7 Hz, 1H), 8.69 (d, J = 5.1 Hz, 1H), 8.52 (dd, J = 7.8, 1.1 Hz, 1H), 8.24-8.15 (m, 2H), 8.12 (t, J = 7.8 Hz, 1H), 8.02 (d, J = 5.1 Hz, 1H), 7.21 (s, 1H), 4.61-4.52 (m, 2H), 3.70-3.56 (m, 2H), 3.00 (s, 5H), 2.86- 2.74 (m, 1H), 2.50- 2.43 (m, 1H), 2.23- 2.13 (m, 2H). MS | RP HPLC (NH₄OH modifier) | 4 |
| | (R)-3-(3-(6-(2-((3,4-Dihydro-2H- | (ESI⁺): m/z = 486.5. | | |
| | pyrano[2,3-b]pyridin-6- | | | |
| | yl)amino)pyrimidin-4-yl)pyridin- | | | |
| | 2-yl)isoxazol-5-yl)-3-hydroxy-1- | | | |
| | methylpyrrolidin-2-one | | | |
| 475 | 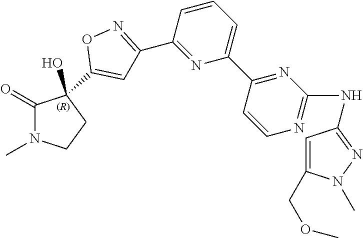 | ¹H NMR (500 MHz, DMSO-d₆) δ 9.94 (s, 1H), 8.71 (d, J = 5.0 Hz, 1H), 8.53 (dd, J = 7.6, 1.2 Hz, 1H), 8.31- 8.14 (m, 2H), 7.88 (dd, J = 5.0, 1.0 Hz, 1H), 7.23 (d, J = 0.7 Hz, 1H), 6.82 (d, J = 8.2 Hz, 2H), 4.55 (s, 2H), 3.78 (s, 3H), 3.53 (dddd, J = 25.9, 9.9, 7.9, 5.3 Hz, 2H), 2.91 (s, 3H), 2.69 (ddd, J = 13.0, 7.6, 4.9 Hz, 1H), | RP HPLC (NH₄OH modifier) | 41 |
| | (R)-3-Hydroxy-3-(3-(6-(2-((5- | 2.42-2.29 (m, 1H). | | |
| | (methoxymethyl)-1-methyl-1H- | MS (ESI⁺): m/z = | | |
| | pyrazol-3-yl)amino)pyrimidin-4- | 477.3. | | |
| | yl)pyridin-2-yl)isoxazol-5-yl)-1- | | | |
| | methylpyrrolidin-2-one | | | |
| 476 | 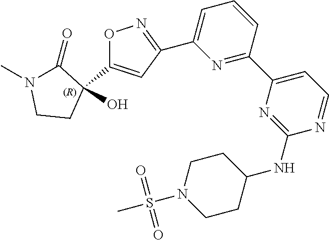 | ¹H NMR (500 MHz, Methanol-d₄) δ 8.57- 8.43 (m, 2H), 8.15 (dd, J = 7.8, 1.1 Hz, 1H), 8.07 (t, J = 7.8 Hz, 1H), 7.75 (d, J = 5.1 Hz, 1H), 7.19 (s, 1H), 4.11 (s, 1H), 3.77 (d, J = 12.3 Hz, 2H), 3.65- 3.59 (m, 2H), 2.99 (s, 5H), 2.90 (s, 3H), 2.83- 2.75 (m, 1H), 2.49- 2.39 (m, 1H), 2.20 (d, J = 13.0 Hz, 2H), 1.82- 1.65 (m, 2H). MS | RP HPLC (NH₄OH modifier) | 7 |
| | (R)-3-Hydroxy-1-methyl-3-(3- | (ESI⁺): m/z = 514.2. | | |
| | (6-(2-((1- | | | |
| | (methylsulfonyl)piperidin-4- | | | |
| | yl)amino)pyrimidin-4-yl)pyridin- | | | |
| | 2-yl)isoxazol-5-yl)pyrrolidin-2- | | | |
| | one | | | |

Compounds of the present disclosure were tested in biological assays. The results of the assays are presented in Table 10 below which is entitled Results of Biological Assays.

### Assay 1: Inhibition of Auto-Phosphorylation of Recombinant Human NF-kappaB-Inducing Kinase (NIK/MAP3K14) Activity (AlphaScreen™)

NIK/MAP3K14 auto-phosphorylation activity was measured using the AlphaScreen™ (αscreen) format (Perkin Elmer). All compounds tested were dissolved in dimethyl sulfoxide (DMSO) and further dilutions were made in assay buffer. The final DMSO concentration was 0.7% (v/v) in assays. The assay buffer was 50 mM Tris pH 7.5 containing 1 mM EGTA (ethylene glycol tetraacetic acid), 1 mM DTT (dithiothreitol), 0.1 mM Na₃VO₄, 5 mM MgCl₂, and 0.01% Tween®20. The assays were carried out in 384 well Proxiplates (Perkin Elmer). The incubations consisted of the compound, 5 μM Adenosine-5′-triphosphate (ATP), and 1 nM NIK/MAP3K14. Incubations were initiated by the addition of GST-tagged NIK/MAP3K14 enzyme, carried out for 2 h at 25° C. and terminated by addition of stop buffer containing anti-phospho-IKK Ser176/180 antibody. Protein A Acceptor and Glutathione-Donor beads were added before reading using an EnVision Multilabel Plate Reader (Perkin Elmer). The signal obtained in the wells was normalized using high (full enzyme activity, 0.7% DMSO) and low controls (no enzyme activity, 0.7% DMSO, no ATP). IC₅₀'s were determined by fitting a sigmoidal curve to % inhibition of control versus Log₁₀ compound concentration.

Assay 2: Effect of Compounds on p-IKKa Levels in L363 (NIK Translocated Multiple Myeloma) Cells

All compounds tested were dissolved and serially diluted in DMSO, 1:3 dilution for 11 points in an Echo compatible plate. 100% DMSO was added to columns 12 and 24 of the plate to serve as high and low signal controls. This compound plate was used to spot 20 nL of compound or DMSO into a Greiner 384 well TC plate (781080). The final DMSO concentration was 0.3% (v/v) in cell assays. Human L363 cells (ATCC) were cultured in RPM11640 medium supplemented with GlutaMax, non-essential amino acids, sodium pyruvate and 10% fetal bovine serum. Cells were routinely maintained at densities of 0.2×10⁶ cells per ml—2×10⁶ cells per mL at 37° C. in a humidified 5% CO₂ atmosphere incubator. Cells were passaged twice a week splitting back to obtain the low density. The day before the assay, cells were washed twice in HBSS (Hank's Balanced Salt Solution), resuspended in Dulbecco's Modified Eagle Medium (DMEM)+0.5% IgG and protease free BSA (Jackson Immuno Research Laboratories), +/−250 ng/ml recombinant human B-cell activating factor (BAFF/BLyS/TNFSF13B) and incubated overnight at 37° C. in a humidified 5% CO₂ atmosphere (bulk stimulation with or without BAFF). The next day, the cell concentration was adjusted to 1×10⁷ cells/ml in DMEM+/−250 ng/ml BAFF +/−10 μM MG132 and plated at 10 μl/well into compound or DMSO spotted 384 well TC plates. Seeded cells were incubated at 37° C. in a humidified 5% CO₂ atmosphere for 6 h. After 6 h, the plates were removed from the incubator and cell lysis was achieved by the addition of 2.5 μl 5×lysis buffer containing protease and phosphatase inhibitors, followed by shaking on a plate shaker at room temperature for 15 min. At the end of this incubation, lysed cells were sequentially treated and incubated with acceptor and donor bead mixes according to the manufacturer's protocol for a 1 plate/2-incubation suspension cell assay (AlphaLISA SureFire Ultra p-IKKa (Ser 176/180) Assay Kit (Perkin Elmer). Plates were read using an EnVision Multilabel Plate Reader (Perkin Elmer). Within an experiment, a concentration response curve for each compound was run in duplicate. The signal obtained in the test wells was normalized using high signal (BAFF stimulated cells, DMSO, MG132) and low signal (unstimulated cells, DMSO) controls. To determine the IC₅₀, a sigmoidal curve was fitted to the plot of % inhibition versus Log₁₀ compound concentration.

Table 10 below provides IC₅₀ data for certain compounds described herein on NIK inhibition. In cases where the compound was tested more than once, the IC₅₀ value was calculated as a simple average of the measured values. The average was then used to categorize the compound within one of the following ranges.

Assay IC₅₀ ranges:

$$A < 50 nM , \\ 50 nM \leq B \leq 500 nM , \\ C > 500 nM .$$

TABLE 10
Results of Biological Assays

| | Assay 1 | Assay 2 |
|---|---|---|
| Example | IC₅₀ (nM) | IC₅₀ (nM) |
| 1 | A | B |
| 2 | A | A |
| 3 | A | A |
| 4 | A | A |
| 5 | A | A |
| 6 | A | B |
| 7 | A | A |
| 8 | A | A |
| 9 | A | B |
| 10 | A | B |
| 11 | B | C |
| 12 | B | C |
| 13 | C | C |
| 14 | C | C |
| 15 | A | C |
| 16 | C | C |
| 17 | A | B |
| 18 | A | C |
| 19 | C | C |
| 20 | C | C |
| 21 | B | C |
| 22 | A | C |
| 23 | A | B |
| 24 | A | B |
| 25 | A | B |
| 26 | A | A |
| 27 | A | A |
| 28 | A | A |
| 29 | A | A |
| 30 | A | A |
| 31 | B | C |
| 32 | C | C |
| 33 | A | C |
| 34 | A | B |
| 35 | B | C |
| 36 | A | A |
| 37 | B | C |
| 38 | A | A |
| 39 | A | C |
| 40 | A | A |
| 41 | A | A |
| 42 | A | A |
| 43 | B | C |
| 44 | A | B |
| 45 | B | C |
| 46 | A | A |
| 47 | A | A |
| 48 | A | A |
| 49 | A | A |
| 50 | A | A |
| 51 | nt | nt |
| 52 | A | A |
| 53 | A | A |
| 54 | A | A |
| 55 | A | A |
| 56 | A | A |
| 57 | A | A |
| 58 | A | A |
| 59 | A | A |
| 60 | A | A |
| 61 | A | A |
| 62 | A | A |
| 63 | A | A |
| 64 | A | A |
| 65 | A | B |
| 66 | A | B |
| 67 | A | B |
| 68 | A | B |
| 69 | A | A |
| 70 | B | C |
| 71 | B | C |
| 72 | A | C |
| 73 | A | A |
| 74 | A | A |
| 75 | A | A |
| 76 | B | C |
| 77 | A | B |
| 78 | C | C |
| 79 | A | C |
| 80 | C | C |
| 81 | C | C |
| 82 | C | C |
| 83 | A | B |
| 84 | C | C |
| 85 | A | B |
| 86 | A | A |
| 87 | C | C |
| 88 | A | B |
| 89 | A | A |
| 90 | A | B |
| 91 | A | A |
| 92 | A | B |
| 93 | A | B |
| 94 | A | B |
| 95 | A | B |
| 96 | A | B |
| 97 | A | B |
| 98 | A | B |
| 99 | A | A |
| 100 | A | A |
| 101 | A | C |
| 102 | A | B |
| 103 | B | C |
| 104 | B | C |
| 105 | A | B |
| 106 | C | C |
| 107 | B | C |
| 108 | A | B |
| 109 | A | A |
| 110 | A | A |
| 111 | A | A |
| 112 | A | B |
| 113 | A | A |
| 114 | A | A |
| 115 | A | B |
| 116 | A | B |
| 117 | A | A |
| 118 | A | C |
| 119 | A | A |
| 120 | A | — |
| 121 | A | A |
| 122 | A | A |
| 123 | B | C |
| 124 | A | A |
| 125 | A | B |
| 126 | A | A |
| 127 | A | B |
| 128 | A | A |
| 129 | A | B |
| 130 | A | B |
| 131 | A | B |
| 132 | A | A |
| 133 | A | A |
| 134 | A | A |
| 135 | B | A |
| 136 | A | A |
| 137 | A | A |
| 138 | A | B |
| 139 | A | B |
| 140 | A | A |
| 141 | A | A |
| 142 | A | B |
| 143 | A | B |
| 144 | A | A |
| 145 | A | A |
| 146 | A | A |
| 147 | A | B |
| 148 | A | A |
| 149 | A | B |
| 150 | A | B |
| 151 | A | B |
| 152 | A | B |
| 153 | B | C |
| 154 | A | B |
| 155 | A | B |
| 156 | A | B |
| 157 | A | C |
| 158 | B | C |
| 159 | A | B |
| 160 | B | C |
| 161 | A | B |
| 162 | A | C |
| 163 | A | B |
| 164 | B | C |
| 165 | A | B |
| 166 | B | C |
| 167 | A | C |
| 168 | A | B |
| 169 | A | C |
| 170 | A | B |
| 171 | A | B |
| 172 | A | B |
| 173 | A | C |
| 174 | A | B |
| 175 | A | C |
| 176 | A | B |
| 177 | A | C |
| 178 | A | B |
| 179 | A | B |
| 180 | A | B |
| 181 | B | C |
| 182 | A | B |
| 183 | A | B |
| 184 | A | B |
| 185 | A | C |
| 186 | A | B |
| 187 | A | A |
| 188 | A | A |
| 189 | A | A |
| 190 | A | A |
| 191 | A | A |
| 192 | A | A |
| 193 | A | A |
| 194 | A | A |
| 195 | A | A |
| 196 | A | A |
| 197 | A | A |
| 198 | A | A |
| 199 | A | A |
| 200 | A | A |
| 201 | A | A |
| 202 | A | A |
| 203 | A | B |
| 204 | A | B |
| 205 | A | A |
| 206 | A | A |
| 207 | C | C |
| 208 | B | C |
| 209 | A | A |
| 210 | A | A |
| 211 | A | A |
| 212 | A | A |
| 213 | A | B |
| 214 | A | A |
| 215 | A | C |
| 216 | A | A |
| 217 | A | A |
| 218 | B | C |
| 219 | B | C |
| 220 | A | B |
| 221 | A | A |
| 222 | A | A |
| 223 | A | A |
| 224 | A | A |
| 225 | A | A |
| 226 | A | A |
| 227 | A | A |
| 228 | A | A |
| 229 | A | A |
| 230 | A | A |
| 231 | A | A |
| 232 | A | B |
| 233 | A | A |
| 234 | A | A |
| 235 | A | A |
| 236 | A | B |
| 237 | A | A |
| 238 | A | A |
| 239 | A | B |
| 240 | A | B |
| 241 | A | B |
| 242 | A | B |
| 243 | A | A |
| 244 | A | B |
| 245 | A | C |
| 246 | B | C |
| 247 | B | C |
| 248 | B | B |
| 249 | A | A |
| 250 | A | A |
| 251 | A | A |
| 252 | A | B |
| 253 | A | A |
| 254 | A | B |
| 255 | A | B |
| 256 | A | B |
| 257 | A | A |
| 258 | A | A |
| 259 | A | A |
| 260 | A | A |
| 261 | A | A |
| 262 | A | A |
| 263 | A | A |
| 264 | A | A |
| 265 | A | C |
| 266 | B | C |
| 267 | A | B |
| 268 | B | C |
| 269 | A | A |
| 270 | A | A |
| 271 | B | C |
| 272 | A | C |
| 273 | C | C |
| 274 | A | B |
| 275 | A | ND |
| 276 | B | C |
| 277 | A | B |
| 278 | B | C |
| 279 | A | B |
| 280 | A | B |
| 281 | B | C |
| 282 | A | A |
| 283 | A | A |
| 284 | A | A |
| 285 | A | A |
| 286 | C | C |
| 287 | A | B |
| 288 | A | A |
| 289 | A | C |
| 290 | A | B |
| 291 | A | B |
| 292 | A | A |
| 293 | A | A |
| 294 | A | A |
| 295 | A | A |
| 296 | A | A |
| 297 | A | A |
| 298 | A | A |
| 299 | A | A |
| 300 | A | A |
| 301 | A | A |
| 302 | A | A |
| 303 | A | B |
| 304 | A | B |
| 305 | A | B |
| 306 | A | C |
| 307 | A | B |
| 308 | A | B |
| 309 | B | C |
| 310 | B | C |
| 311 | A | A |
| 312 | A | A |
| 313 | A | C |
| 314 | A | C |
| 315 | B | C |
| 316 | B | C |
| 317 | B | C |
| 318 | B | C |
| 319 | A | C |
| 320 | A | A |
| 321 | B | C |
| 322 | A | B |
| 323 | A | B |
| 324 | A | C |
| 325 | A | B |
| 326 | A | A |
| 327 | A | A |
| 328 | A | B |
| 329 | A | A |
| 330 | A | A |
| 331 | B | C |
| 332 | A | B |
| 333 | A | B |
| 334 | A | A |
| 335 | A | A |
| 336 | A | C |
| 337 | A | C |
| 338 | B | C |
| 339 | A | B |
| 340 | A | B |
| 341 | A | B |
| 342 | A | B |
| 343 | A | B |
| 344 | A | B |
| 345 | A | B |
| 346 | A | B |
| 347 | A | B |
| 348 | A | B |
| 349 | A | B |
| 350 | A | C |
| 351 | A | B |
| 352 | A | B |
| 353 | A | B |
| 354 | A | B |
| 355 | A | C |
| 356 | A | B |
| 357 | B | C |
| 358 | B | C |
| 359 | A | B |
| 360 | A | C |
| 361 | A | B |
| 362 | A | B |
| 363 | A | B |
| 364 | A | B |
| 365 | A | C |
| 366 | A | B |
| 367 | A | B |
| 368 | A | B |
| 369 | A | C |
| 370 | A | C |
| 371 | A | B |
| 372 | A | B |
| 373 | A | B |
| 374 | A | B |
| 375 | A | B |
| 376 | A | B |
| 377 | A | C |
| 378 | A | B |
| 379 | A | B |
| 380 | A | B |
| 381 | A | B |
| 382 | A | B |
| 383 | A | B |
| 384 | A | B |
| 385 | A | B |
| 386 | A | B |
| 387 | A | C |
| 388 | A | B |
| 389 | A | B |
| 390 | A | C |
| 391 | A | C |
| 392 | A | C |
| 393 | A | B |
| 394 | B | C |
| 395 | A | B |
| 396 | B | C |
| 397 | B | C |
| 398 | A | C |
| 399 | A | A |
| 400 | B | C |
| 401 | A | A |
| 402 | A | A |
| 403 | A | A |
| 404 | B | C |
| 405 | A | B |
| 406 | A | A |
| 407 | A | A |
| 408 | A | A |
| 409 | A | A |
| 410 | A | A |
| 411 | A | B |
| 412 | A | B |
| 413 | A | B |
| 414 | A | C |
| 415 | B | C |
| 416 | A | C |
| 417 | B | C |
| 418 | B | C |
| 419 | A | C |
| 420 | A | C |
| 421 | A | C |
| 422 | C | C |
| 423 | B | C |
| 424 | C | C |
| 425 | B | C |
| 426 | C | C |
| 427 | C | C |
| 428 | A | B |
| 429 | A | B |
| 430 | A | B |
| 431 | A | B |
| 432 | A | A |
| 433 | A | A |
| 434 | A | B |
| 435 | A | B |
| 436 | A | B |
| 437 | A | B |
| 438 | A | B |
| 439 | A | B |
| 440 | A | B |
| 441 | A | C |
| 442 | A | B |
| 443 | A | B |
| 444 | A | A |
| 445 | A | C |
| 446 | A | B |
| 447 | A | B |
| 448 | A | C |
| 449 | A | B |
| 450 | A | B |
| 451 | A | C |
| 452 | A | B |
| 453 | A | B |
| 454 | A | B |
| 455 | B | C |
| 456 | A | B |
| 457 | A | B |
| 458 | A | B |
| 459 | A | B |
| 460 | A | B |
| 461 | A | B |
| 462 | A | B |
| 463 | A | B |
| 464 | A | A |
| 465 | A | B |
| 466 | A | C |
| 467 | A | B |
| 468 | A | A |
| 469 | A | C |
| 470 | A | A |
| 471 | A | A |
| 472 | A | B |
| 473 | A | B |
| 474 | A | A |
| 475 | A | A |
| 476 | A | B |

1. A compound of Formula I′: 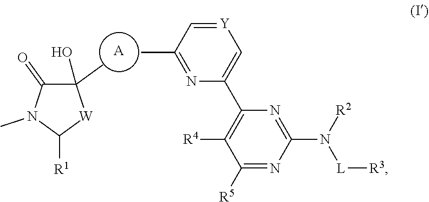
or a pharmaceutically acceptable salt thereof, wherein:
A is a 5-membered heteroaryl that is optionally substituted with one or more —C₍₁₋₄₎alkyl groups;
W is CH₂, CHF, CF₂, or CHR^{W};
X is N, C—H, or C—R^{X};
Y is N, C—H, or C—R^{Y};
R^{W} is —C₍₁₋₄₎alkyl or —C₍₁₋₄₎haloalkyl;
R^{X} is halo, —C₍₁₋₄₎alkyl, —C₍₁₋₄₎haloalkyl, —OC₍₁₋₄₎alkyl, —OC₍₁₋₄₎haloalkyl, or —C₍₁₋₄₎alkyl-O—C₍₁₋₄₎alkyl;
R^{Y} is halo, —C₍₁₋₄₎alkyl, —C₍₁₋₄₎haloalkyl, —OC₍₁₋₄₎alkyl, —OC₍₁₋₄₎ haloalkyl, or —C₍₁₋₄₎alkyl-O—C₍₁₋₄₎alkyl;
R¹ is hydrogen, —C₍₁₋₄₎alkyl, or —C₁₋₄₎haloalkyl; or wherein R^{W} and R¹ are taken together with the carbon atoms to which they are attached to form a C₍₃₋₅₎cycloalkyl;
R² is hydrogen or —C₍₁₋₄₎alkyl;
L is absent, —C₍₁₋₄₎alkylene, or —C₍₃₋₆₎cycloalkylene, wherein the —C₍₁₋₄₎alkylene and —C₍₃₋₆₎cycloalkylene are optionally substituted with one to three groups selected from halo, —OH, —N(R^{N1})(R^{N2}), —C₍₁₋₃₎alkyl, —C₍₁₋₃₎haloalkyl, —C₍₃₋₅₎cycloalkyl, and —OC₍₁₋₃₎alkyl;
R³ is —C₍₁₋₁₀₎alkyl, —C₍₃₋₁₀₎cycloalkyl, 3- to 10-membered heterocyclyl, a 5- to 12-membered bi- or tricyclic ring system containing one or more heteroatoms, —C₍₆₋₁₀₎aryl, 5- to 10-membered heteroaryl, or —C(O)N(R^{N3})(R^{N4}), wherein the —C₍₁₋₁₀₎alkyl, —C₍₃₋₁₀₎cycloalkyl, 3- to 10-membered heterocyclyl, a 5- to 12-membered bi- or tricyclic ring system containing one or more heteroatoms, —C₍₆₋₁₀₎aryl, and 5- to 10-membered heteroaryl are each optionally substituted with one to five R^{3x} groups;
each R^{3x} independently for each occurrence is halo, —OH, —N(R^{N1})(R^{N2}), —CN, —C₍₁₋₈₎alkyl, —C₍₁₋₈₎ haloalkyl, —C₍₃₋₈₎cycloalkyl, —OC₍₁₋₈₎alkyl, —OC₍₁₋₈₎ haloalkyl, —OC₍₃₋₈₎cycloalkyl, —C₍₁₋₈₎alkylC₍₃₋₁₀₎cycloalkyl, —C₍₃₋₁₀₎cycloalkylC₍₁₋₈₎alkyl, —C(O)C₍₁₋₈₎alkyl, —S(O)₂C₍₁₋₈₎alkyl, —S(O)₂C₍₃₋₈₎cycloalkyl, —N(H)S(O)₂C₍₁₋₈₎alkyl, —C₍₀₋₈₎alkylC(O)N(R^{N1})(R^{N2}), 3- to 8-membered heterocyclyl, —C₍₁₋₈₎alkyl(3- to 8-membered heterocyclyl), —C₍₆₋₁₀₎aryl, or 5- to 10-membered heteroaryl, wherein the —C₍₁₋₈₎alkyl, —C₍₁₋₈₎ haloalkyl, —C₍₃₋₈₎cycloalkyl, —OC₍₁₋₈₎alkyl, —OC₍₁₋₈₎haloalkyl, —OC₍₃₋₈₎cycloalkyl, —C₍₁₋₈₎alkylC₍₃₋₁₀₎cycloalkyl, —C₍₃₋₁₀₎cycloalkylC₍₁₋₈₎alkyl, —C(O)C₍₁₋₈₎alkyl, —S(O)₂C₍₁₋₈₎alkyl, —S(O)₂C₍₃₋₈₎cycloalkyl, —N(H)S(O)₂C₍₁₋₈₎alkyl, 3- to 8-membered heterocyclyl, —C₍₁₋₈₎alkyl(3- to 8-membered heterocyclyl), —C₍₆₋₁₀₎aryl, and 5- to 10-membered heteroaryl are optionally further substituted with one to five groups selected from halo, —OH, —N(R^{N1})(R^{N2}), —CN, —C₍₁₋₈₎alkyl, —C₍₁₋₈₎ haloalkyl, —OC₍₁₋₈₎alkyl, —OC₍₁₋₈₎haloalkyl, and 3- to 5-membered heterocyclyl; or
R² and L-R³ are combined, along with the nitrogen atom to which they are attached, to form a 3- to 10-membered heterocyclyl, a 5- to 12-membered bicyclic ring system containing one or more heteroatoms, or a 5- to 10-membered heteroaryl, each of which is optionally further substituted with one to three groups selected from halo, —OH, —N(R^{N1})(R^{N2}), —CN, —C₍₁₋₈₎alkyl, and —OC₍₁₋₈₎alkyl wherein the —C₍₁₋₈₎alkyl and —OC₍₁₋₈₎alkyl are optionally further substituted with one to five halo groups;
R⁴ is hydrogen, halo, or —C₍₁₋₄₎alkyl;
R⁵ is hydrogen, halo, or —C₍₁₋₄₎alkyl;
R^{N1} and R^{N2} are each independently for each occurrence hydrogen, —C₍₁₋₈₎alkyl, or —C₍₁₋₈₎ haloalkyl;
R^{N3} is hydrogen or —C₍₁₋₈₎alkyl; and
R^{N4} is hydrogen, —C₍₁₋₈₎alkyl, or phenyl; or R^{N3} and R^{N4} taken together with the nitrogen atom to which they are attached form a 3- to 10-membered heterocyclyl that is optionally substituted with one to five groups selected from halo, —OH, —N(R^{N1})(R^{N2}), —CN, —C₍₁₋₈₎alkyl, —C₍₁₋₈₎ haloalkyl, —OC₍₁₋₈₎alkyl, and —OC₍₁₋₈₎ haloalkyl;
wherein if R³ is —C(O)N(R^{N3})(R^{N4}), then L is not absent.

2-10. (canceled)

11. The compound of any one of claim 1, or a pharmaceutically acceptable salt thereof, wherein:
A is pyrazolyl, triazolyl, thiazolyl, or isoxazolyl;
W is CH₂ or CF₂;
Y is C—H or C—R^{Y};
R^{Y} is —CF₃;
R¹ is hydrogen or —CF₃;
R² is hydrogen;
L is absent, —C₍₁₋₄₎alkylene, or —C₍₃₋₆₎cycloalkylene;
R³ is —C₍₁₋₆₎alkyl, —C₍₃₋₁₀₎cycloalkyl, 3- to 10-membered heterocyclyl, a 5- to 12-membered bi- or tricyclic ring system containing one or more heteroatoms, or 5- to 10-membered heteroaryl; wherein: the —C₍₁₋₆₎alkyl is optionally substituted with one to five fluorine atoms; the —C₍₃₋₁₀₎cycloalkyl is optionally substituted with one —OH group; the 5- to 12-membered bi- or tricyclic ring system containing one or more heteroatoms is optionally substituted with one to five —C₍₁₋₆₎alkyl groups, and the 5- to 10-membered heteroaryl is optionally substituted with one to five R^{3f} groups;
each R^{3f} is independently for each occurrence —N(R^{N1})(R^{N2}), —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, 3- to 8-membered heterocyclyl, or —C₍₁₋₃₎alkyl(3- to 8-membered heterocyclyl), wherein the —C₍₁₋₆₎alkyl, —C₍₁₋₆₎haloalkyl, —C₍₃₋₈₎cycloalkyl, —OC₍₁₋₆₎alkyl, —OC₍₁₋₆₎haloalkyl, —OC₍₃₋₈₎cycloalkyl, 3- to 8-membered heterocyclyl, and —C₍₁₋₃₎alkyl(3- to 8-membered heterocyclyl) are optionally further substituted with one to five groups selected from halo, —OH, —C₍₁₋₄₎alkyl, —C₍₁₋₄₎haloalkyl, —OC₍₁₋₄₎alkyl, and —OC₍₁₋₄₎ haloalkyl; or
R² and L-R³ are combined, along with the nitrogen atom to which they are attached, to form a 5- to 12-membered bicyclic ring system containing one or more heteroatoms or a 5- to 10-membered heteroaryl, each of which that is optionally further substituted with one to three groups selected from —N(R^{N1})(R^{N2}) and —C₍₁₋₆₎alkyl; and
R^{N1} and R^{N2} are each independently for each occurrence hydrogen or —C₍₁₋₃₎alkyl.

12. The compound of claim 1, or a pharmaceutically acceptable salt thereof, wherein A is triazolyl or isoxazolyl.

13. The compound of claim 1, or a pharmaceutically acceptable salt thereof, wherein A is isoxazolyl.

14. (canceled)

15. The compound of claim 1, or a pharmaceutically acceptable salt thereof, which is a compound of Formula Ib-1:

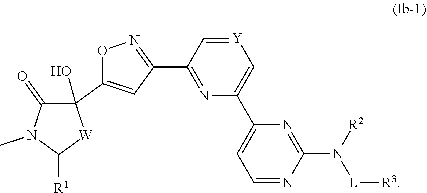

16-17. (canceled)

18. The compound of claim 1, or a pharmaceutically acceptable salt thereof, wherein X is N, C—H, or C—F.

19. (canceled)

20. The compound of claim 1, or a pharmaceutically acceptable salt thereof, wherein Y is C—H or C—CF₃.

21-22. (canceled)

23. The compound of claim 1, or a pharmaceutically acceptable salt thereof, wherein R¹ is hydrogen or —CF₃.

24-26. (canceled)

27. The compound of claim 1, or a pharmaceutically acceptable salt thereof, wherein L is absent, —C₍₁₋₄₎alkylene, or cyclopropylene.

28. (canceled)

29. The compound of claim 1, or a pharmaceutically acceptable salt thereof, which is a compound of any one of Formulas Ie-1 to Ie-10:

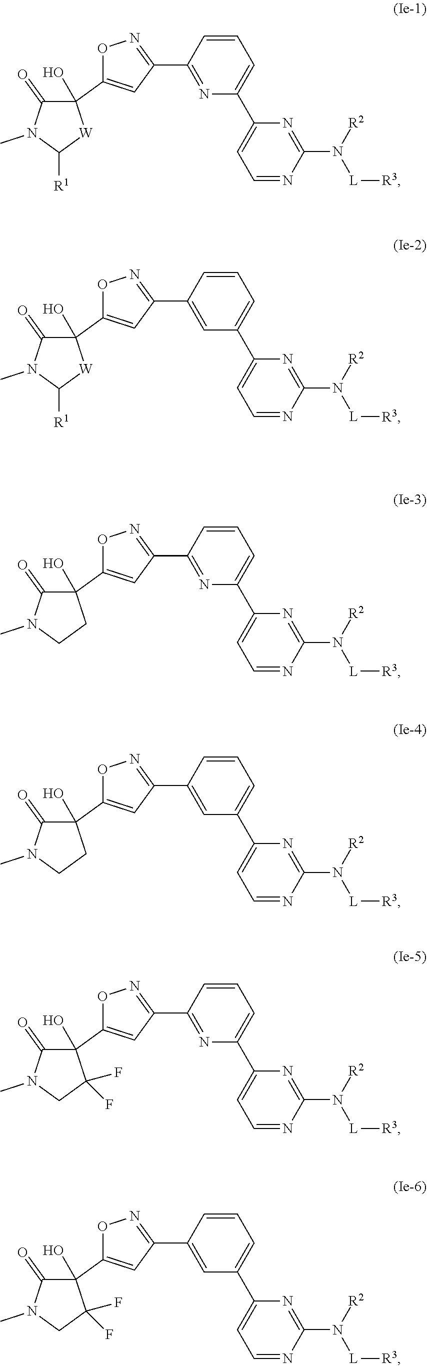

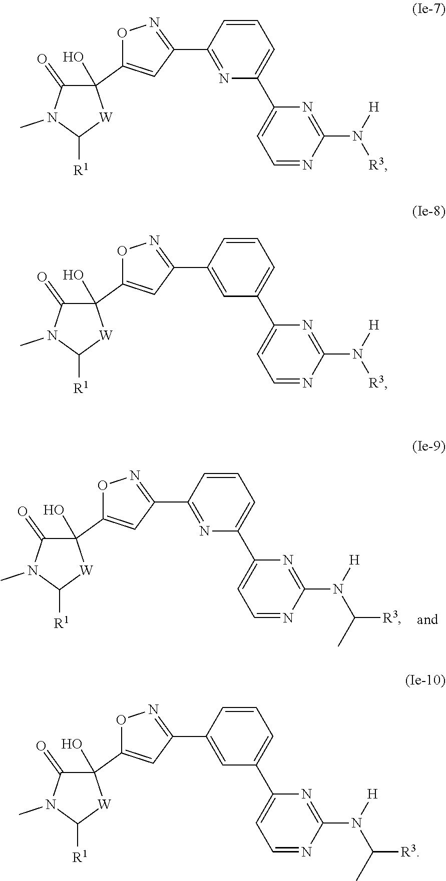

30. The compound of claim 1, or a pharmaceutically acceptable salt thereof, which is a compound of any one of Formulas If-1 to If-10:

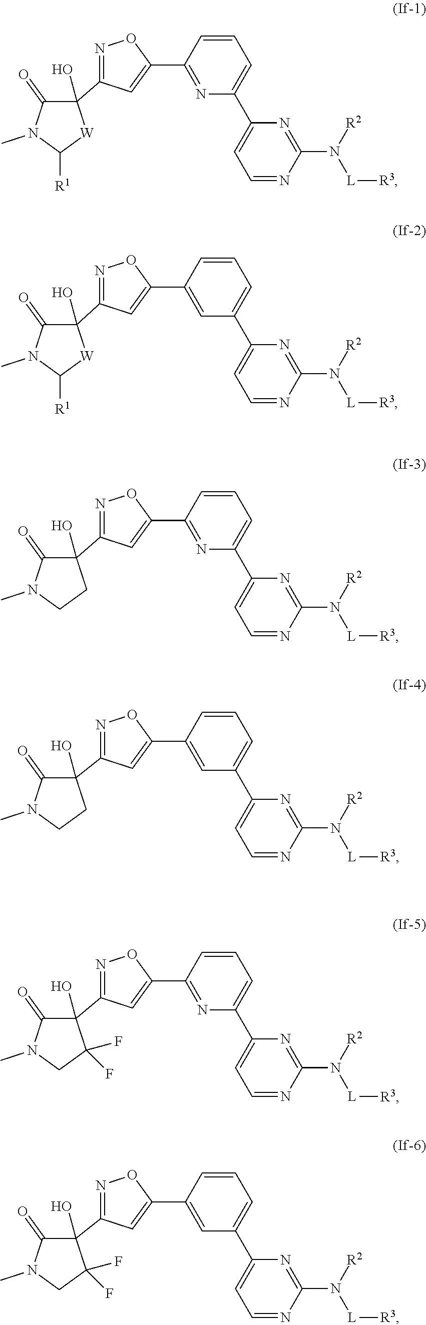

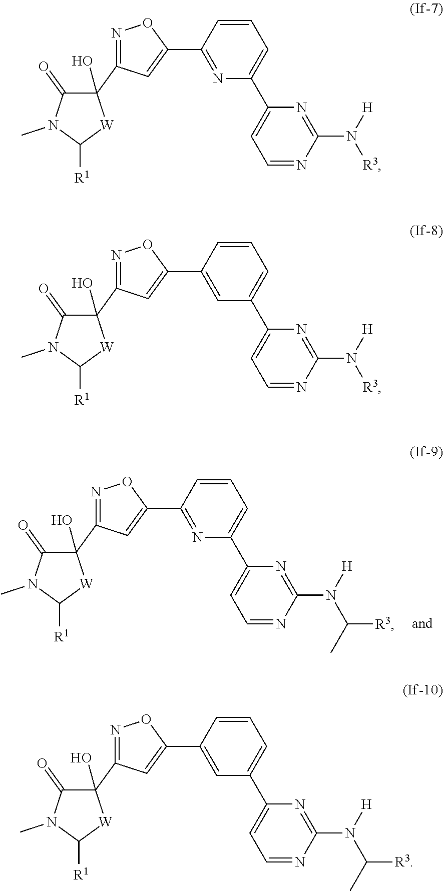

31-56. (canceled)

57. The compound of claim 1, or a pharmaceutically acceptable salt thereof, wherein R³ is 5- to 10-membered heteroaryl, selected from the group of pyrazolyl, isoxazolyl, or pyrazolopyrimidinyl which is optionally substituted with one to five R^{3f} groups.

58-66. (canceled)

67. The compound of claim 1, or a pharmaceutically acceptable salt thereof, wherein each R^{3f} is independently for each occurrence —C₍₁₋₆₎alkyl, —OC₍₃₋₈₎cycloalkyl, —OC₍₁₋₆₎alkyl, or —OC₍₃₋₈₎cycloalkyl, each of which is optionally further substituted with one to five groups selected from fluorine and —OH.

68-85. (canceled)

86. The compound of claim 1, or a pharmaceutically acceptable salt thereof, which is a compound of Formula Ig-1: 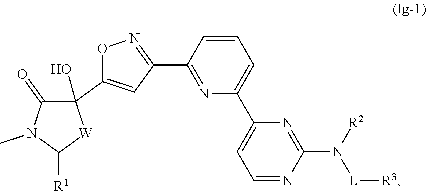
wherein:
W is CH₂ or CF₂;
X is N or C—H;
R¹ is hydrogen;
R² is hydrogen;
L is absent or —C₍₁₋₄₎alkylene;
R³ is 5- to 10-membered heteroaryl, which is optionally substituted with one to five R^{3e} groups;
each R^{3e} is independently —C₍₁₋₆₎alkyl, —OC₍₃₋₈₎cycloalkyl, —OC₍₁₋₆₎alkyl, or —OC₍₃₋₈₎cycloalkyl, each of which is optionally further substituted with one to five groups selected from fluorine and —OH.

87-88. (canceled)

89. The compound of claim 1, or a pharmaceutically acceptable salt thereof, having a structure selected from the group consisting of:

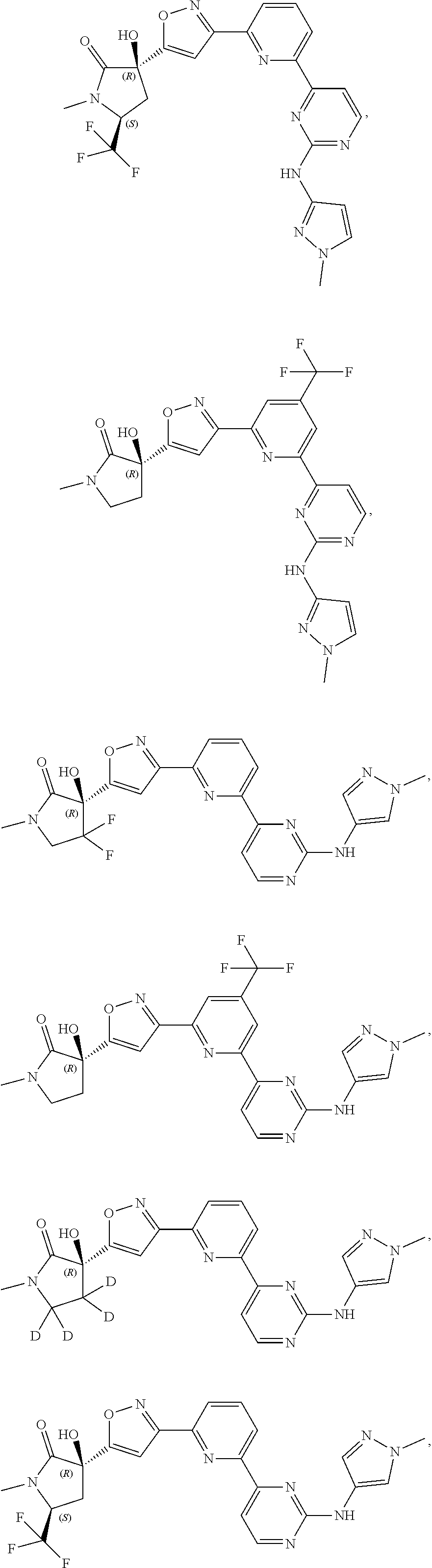

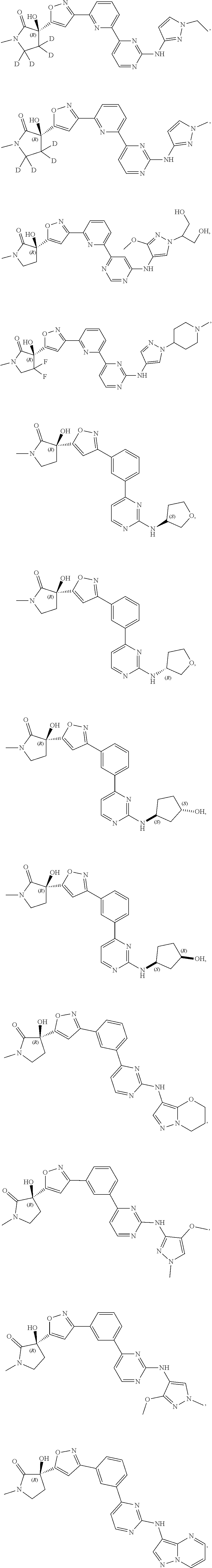

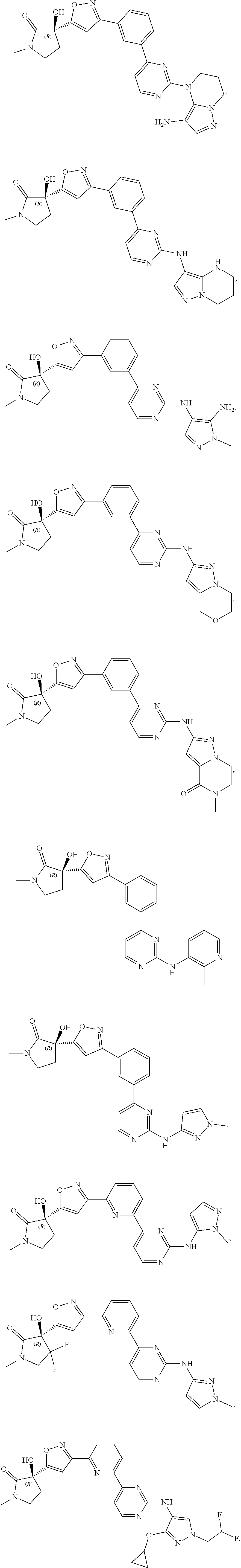

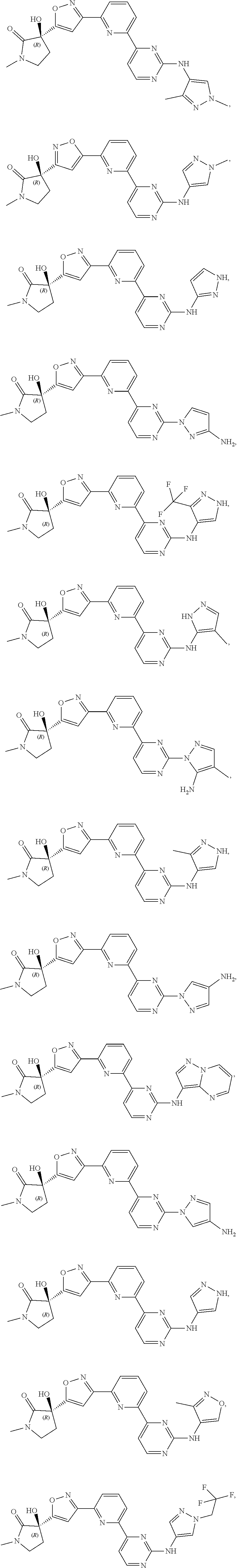

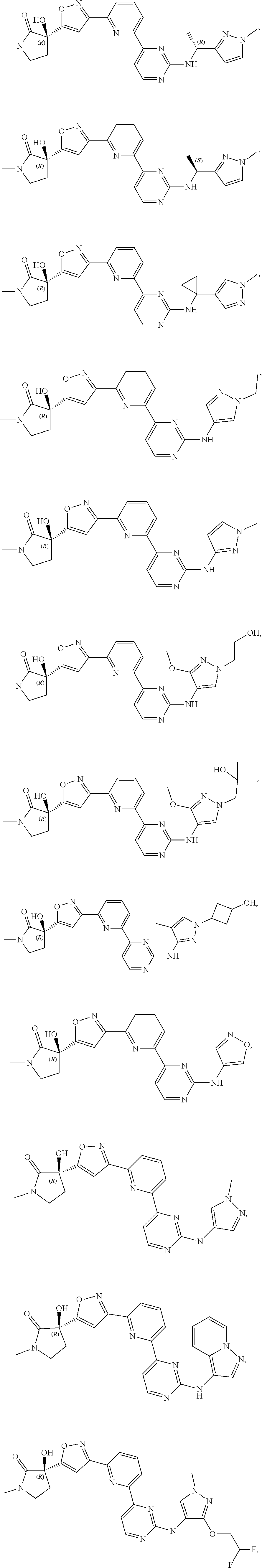

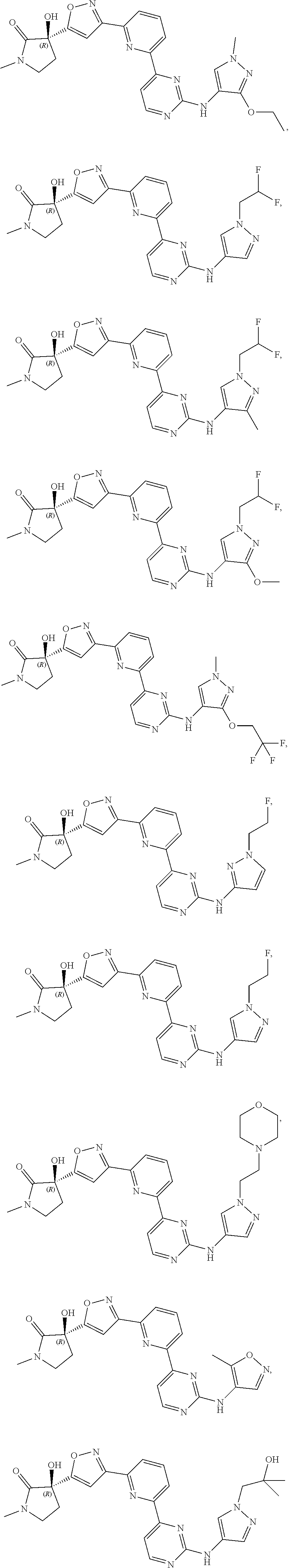

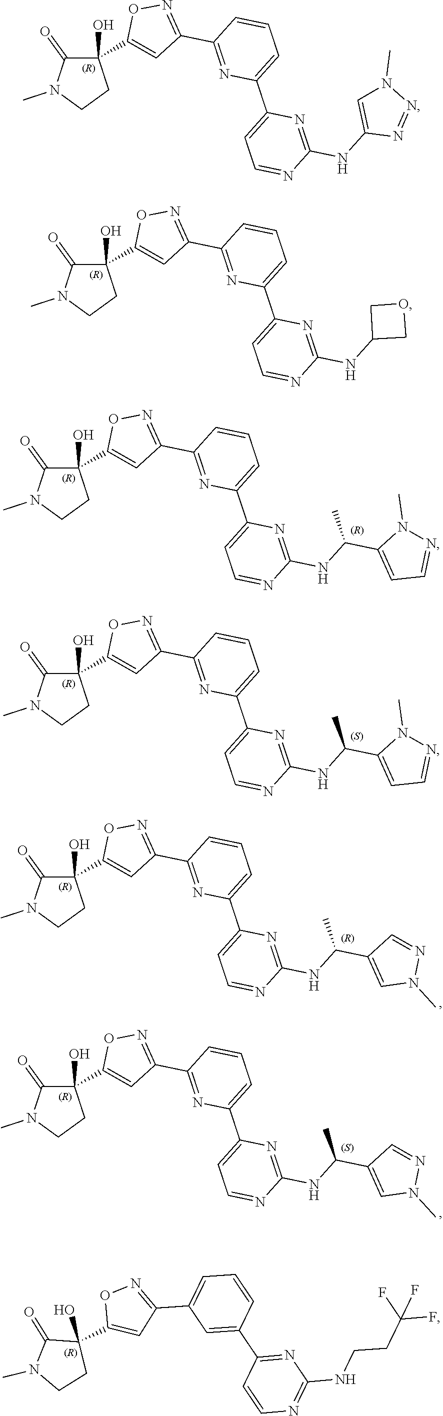

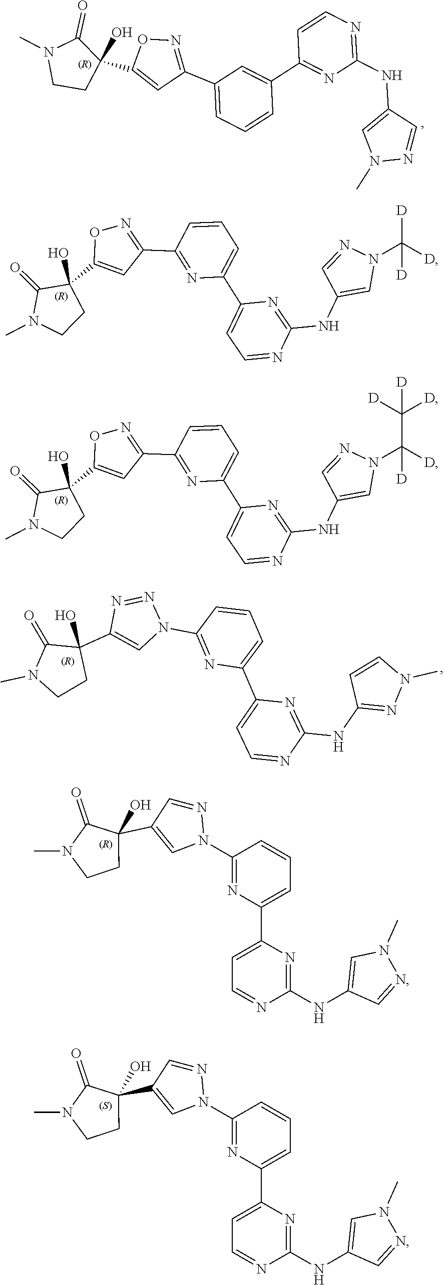

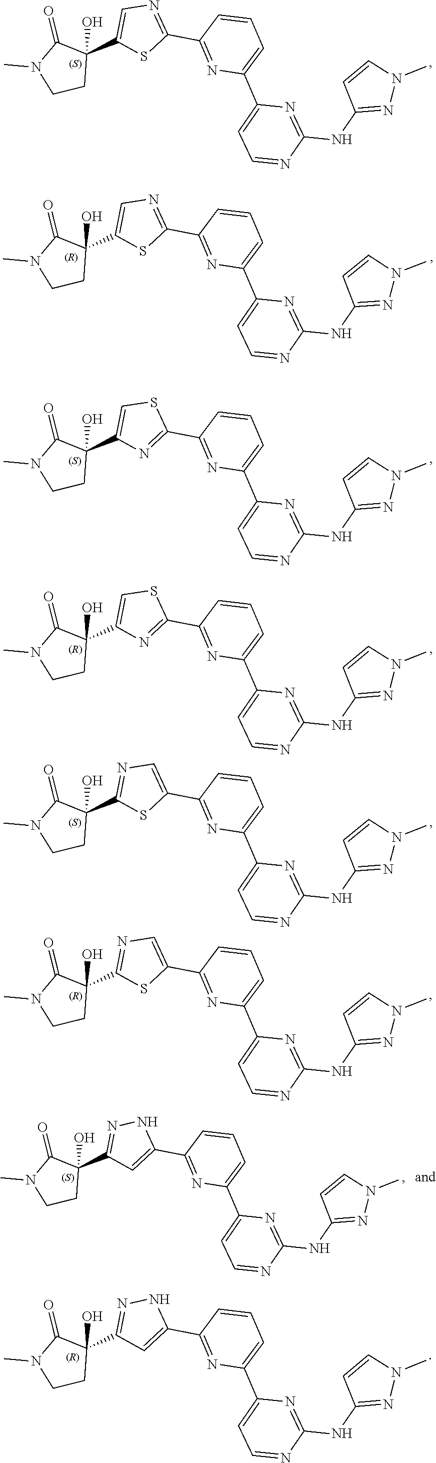

90. (canceled)

91. The compound of claim 1, or a pharmaceutically acceptable salt thereof, having a structure selected from the group consisting of:

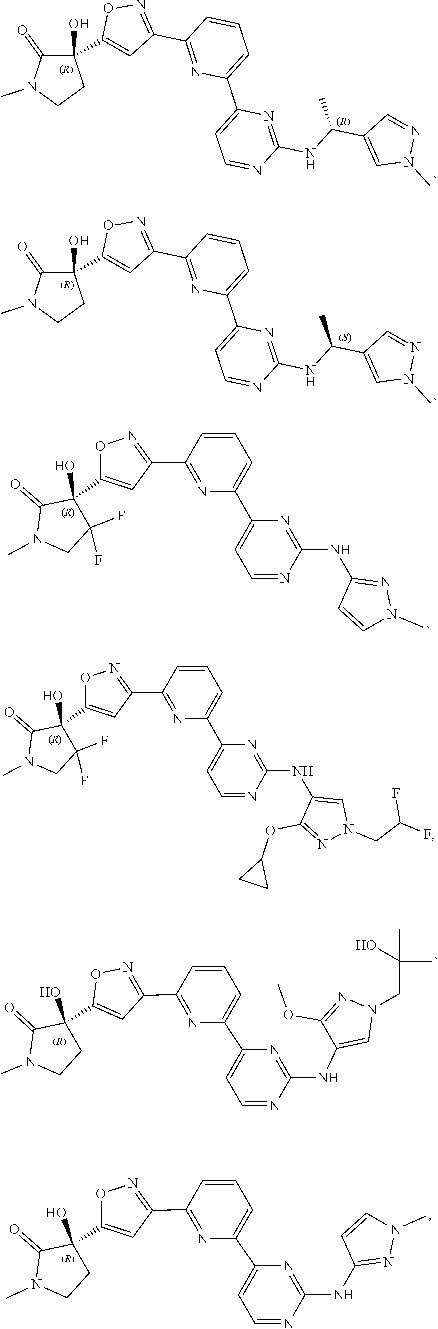

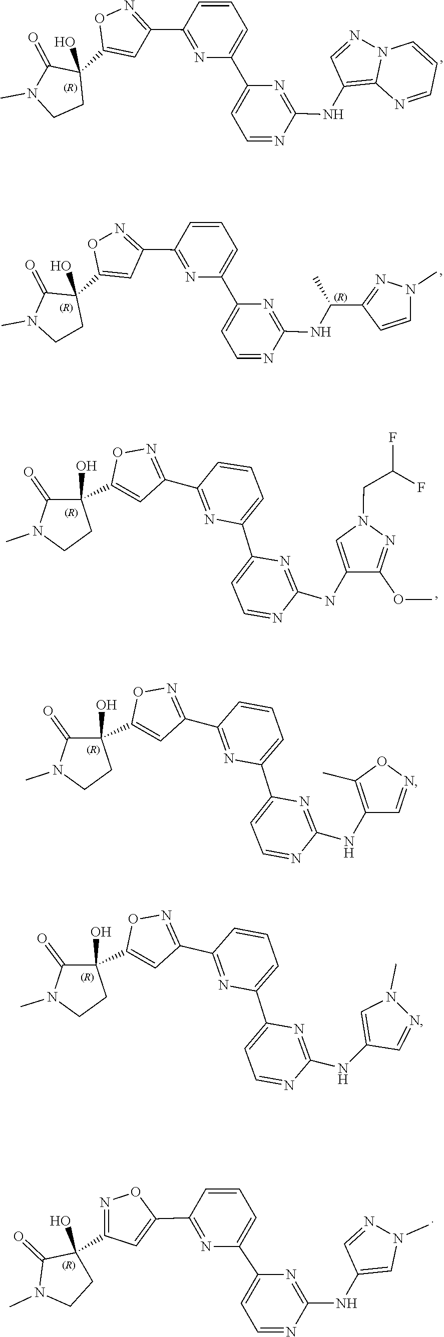

92-111. (canceled)

112. A pharmaceutical composition comprising a compound of claim 1, or a pharmaceutically acceptable salt thereof, and a pharmaceutically acceptable carrier.

113. A method of treating a disease, disorder, or medical condition mediated by NIK activity, comprising administering to a subject in need of such treatment an effective amount of (i) a compound of claim 1, or a pharmaceutically acceptable carrier thereof.

114-115. (canceled)

116. The method of claim 113, wherein the disease, disorder or medical condition mediated by NIK activity is selected from the group consisting of inflammatory disorders, autoimmune disorders, cancers, metabolic disorders, and osteoporosis.

117. The method of claim 113, wherein the disease, disorder, or medical condition mediated by NIK activity is selected from the group consisting of systemic lupus erythematosus (“SLE”), rheumatoid arthritis (“RA”), Sjogren's syndrome, lupus nephritis, inflammatory bowel disease (“IBD”), ANCA associated vasculitis, myositis, IgG4 associated diseases, bullous pemphigoid, neuromyelitis optica spectrum disorders (“NMOSD”), atopic dermatitis “AD”), hidradenitis supperativa (“HS”), steatosis, non-alcoholic steatohepatitis (“NASH”), primary biliary cirrhosis, leukemias, lymphomas, pancreatic cancer, breast cancer, melanoma, obesity, diabetes, acute kidney injury, IgAN, autosomal dominant polycystic kidney disease (“ADCKD”), membranous nephropathy, osteoporosis, bone resorption (periodontitis), multiple sclerosis (“MS”), immune thrombocytopenic purpura, transplantation, myasthenia gravis, scleroderma, myositis, IgG4 associated diseases, and bullous pemphigoid.
